(19) United States
(12) Patent Application Publication
(10) Pub. No.: US 2025/0171756 A1
(43) Pub. Date: May 29, 2025

(54) NOVEL TYPE VI CRISPR ENZYMES AND SYSTEMS

(71) Applicants: THE BROAD INSTITUTE, INC., Cambridge, MA (US); MASSACHUSETTS INSTITUTE OF TECHNOLOGY, Cambridge, MA (US)

(72) Inventors: Feng Zhang, Cambridge, MA (US); Han Altae-Tran, Cambridge, MA (US); Soumya Kannan, Cambridge, MA (US)

(21) Appl. No.: 18/965,935

(22) Filed: Dec. 2, 2024

Related U.S. Application Data
(63) Continuation of application No. 17/761,292, filed on Mar. 17, 2022.
(60) Provisional application No. 62/903,604, filed on Sep. 20, 2019.
(60) Provisional application No. 62/905,645, filed on Sep. 25, 2019.
(60) Provisional application No. 62/967,408, filed on Jan. 29, 2020.
(60) Provisional application No. 63/044,190, filed on Jun. 25, 2020.

(51) Int. Cl. C12N 9/22; C12N 9/78; C12N 15/11; C12N 15/90

(52) U.S. Cl. CPC C12N 9/22; C12N 9/78; C12N 15/11; C12N 15/907; C12Y 305/04004; C12N 2310/20; C12N 2800/80

(57) ABSTRACT

The present disclosure provides for systems, methods, and compositions for targeting nucleic acids. In particular, the invention provides Cas proteins and their use in modifying target sequences.

### CROSS-REFERENCE TO RELATED APPLICATIONS

This application is a continuation of application Ser. No. 17/761,292 filed Mar. 17, 2022, which is a national phase entry of International Application No. PCT/US2020/051660 filed Sep. 18, 2020, which claims the benefit of U.S. Provisional Application No. 62/903,604, filed Sep. 20, 2019, U.S. Provisional Application No. 62/905,645 filed Sep. 25, 2019, U.S. Provisional Application No. 62/967,408, filed Jan. 29, 2020, and U.S. Provisional Application No. 63/044,190 filed Jun. 25, 2020. The entire contents of the above-identified applications are hereby fully incorporated herein by reference.

### STATEMENT REGARDING FEDERALLY SPONSORED RESEARCH

This invention was made with government support under Grant Nos. HG009761, MI 10049, and HL141201 awarded by the National Institutes of Health. The government has certain rights in the invention.

### REFERENCE TO AN ELECTRONIC SEQUENCE LISTING

The contents of the electronic sequence listing (“BROD-4860US-CON_ST26_Revised.xml”; Size is 13,001,447 bytes and it was created on Jan. 30, 2025) is herein incorporated by reference in its entirety.

### TECHNICAL FIELD

The present invention generally relates to systems, methods and compositions used for the control of gene expression involving sequence targeting, such as perturbation of gene transcripts or nucleic acid editing, that may use vector systems related to Clustered Regularly Interspaced Short Palindromic Repeats (CRISPR) and components thereof.

### BACKGROUND

The CRISPR-CRISPR associated (Cas) systems of bacterial and archaeal adaptive immunity are some such systems that show extreme diversity of protein composition and genomic loci architecture. There exists a pressing need for alternative and robust systems and techniques for targeting nucleic acids or polynucleotides.

### SUMMARY

In one aspect, the present disclosure provides a non-naturally occurring or engineered composition comprising: a Cas protein that comprises at least one HEPN domain and is less than 900 amino acids in size; and a guide sequence capable of forming of complex with the Cas protein and directing the complex to bind to a target sequence. In some embodiments, the Cas protein is a Type VI Cas protein. In some embodiments, the Cas protein is Cas13. In some embodiments, the Cas protein is selected from (a) SEQ ID NOs. 4102-4298; (b) SEQ ID NOs. 4299-4654; (c) SEQ ID NOs. 2771-2772, 4655-4768, or 5260-5265; (d) SEQ ID NOs. 4769-4797; or (e) SEQ ID NOs. 4798-5203.

In another aspect, the present disclosure provides a non-naturally occurring or engineered system comprising: (a) a Cas protein selected from: (i) SEQ ID NOs. 1-1323, (ii) SEQ ID NOs. 1324-2770, (iii) SEQ ID NOs. 2773-2797, or (iv) SEQ ID NOs. 2798-4092; (b) a guide sequence capable of forming of complex with the Cas protein and directing the complex to bind to a target sequence.

In some embodiments, the Cas protein exhibits collateral nuclease activity and cleaves a non-target sequence. In some embodiments, the composition comprises two or more guide sequences capable of hybridizing to two different target sequences or different regions of a target sequence. In some embodiments, the guide sequence is capable of hybridizing to one or more target sequences in a prokaryotic cell. In some embodiments, the guide sequence is capable of hybridizing to one or more target sequences in a eukaryotic cell. In some embodiments, the Cas protein comprises one or more nuclear localization signals. In some embodiments, the Cas protein comprises one or more nuclear export signals. In some embodiments, the Cas protein is catalytically inactive. In some embodiments, the Cas protein is a nickase. In some embodiments, the Cas protein is associated with one or more functional domains. In some embodiments, the one or more functional domains is heterologous functional domains. In some embodiments, the one or more functional domains cleaves the one or more target sequences. In some embodiments, the one or more functional domains modifies transcription or translation of the target sequence. In some embodiments, the Cas protein is associated with an adenosine deaminase or cytidine deaminase. In some embodiments, the composition further comprises a recombination template. In some embodiments, the recombination template is inserted by homology-directed repair (HDR). In some embodiments, the composition further comprises a tracr RNA. In some embodiments, the Cas protein comprises two HEPN domains.

In another aspect, the present disclosure provides a non-naturally occurring or engineered composition comprising: an mRNA encoding the Cas protein herein, and a guide sequence capable of forming of complex with the Cas protein and directing the complex to bind to a target sequence.

In another aspect, the present disclosure provides a non-naturally occurring or engineered composition for modifying nucleotides in a target nucleic acid, comprising: the composition herein; and a nucleotide deaminase associated with the Cas protein.

In some embodiments, the Cas protein is a dead Cas protein. In some embodiments, the Cas protein is a nickase. In some embodiments, the nucleotide deaminase is covalently or non-covalently linked to the Cas protein or the guide sequence, or is adapted to link thereof after delivery. In some embodiments, the nucleotide deaminase is a adenosine deaminase. In some embodiments, the nucleotide deaminase is a cytidine deaminase. In some embodiments, the nucleotide deaminase is a human ADAR2 or a deaminase domain thereof. In some embodiments, the adenosine deaminase comprises one or more mutations. In some embodiments, the one or more mutations comprise E620G or Q696L based on amino acid sequence positions of human ADAR2, and corresponding mutations in a homologous ADAR protein. In some embodiments, the adenosine deaminase comprises (i) E488Q and E620G, (ii) E488Q and Q696L, or (iii) E488Q and V505I, based on amino acid sequence positions of human ADAR2, or corresponding mutations in a homologous ADAR protein. In some embodiments, the adenosine deaminase has cytidine deaminase activity. In some embodiments, the nucleotide deaminase protein or catalytic domain thereof has been modified to increase activity against a DNA-RNA heteroduplex. In some embodiments, the nucleotide deaminase protein or catalytic domain thereof has been modified to reduce off-target effects. In some embodiments, the modification of the nucleotides in the target nucleic acid remedies a disease caused by a G→A or C→T point mutation or a pathogenic SNP. In some embodiments, the disease comprises cancer, haemophilia, beta-thalassemia, Marfan syndrome, and Wiskott-Aldrich syndrome. In some embodiments, the modification of the nucleotides in the target nucleic acid remedies a disease caused by a T→C or A→G point mutation or a pathogenic SNP. In some embodiments, the modification of the nucleotide at the target locus of interest inactivates a target gene at the target locus. In some embodiments, the modification of the nucleotide modifies gene product encoded at the target locus or expression of the gene product.

In another aspect, the present disclosure provides an engineered adenosine deaminase comprising one or more mutations: E488Q, E620G, Q696L, or V505I based on amino acid sequence positions of human ADAR2, or corresponding mutations in a homologous ADAR protein. In some embodiments, the adenosine deaminase comprises (i) E488Q and E620G, (ii) E488Q and Q696L, or (iii) E488Q and V505I based on amino acid sequence positions of human ADAR2, or corresponding mutations in a homologous ADAR protein.

In another aspect, the present disclosure provides a system for detecting presence of one or more target polypeptides in one or more in vitro samples comprising: a Cas protein herein;
one or more detection aptamers, each designed to bind to one of the one or more target polypeptides, each detection aptamer comprising a masked promoter binding site or masked primer binding site and a trigger sequence template; and an oligonucleotide-based masking construct comprising a non-target sequence. In some embodiments, the system further comprises nucleic acid amplification reagents to amplify the target sequence or the trigger sequence. In some embodiments, the nucleic acid amplification reagents are isothermal amplification reagents.

In another aspect, the present disclosure provides a system for detecting the presence of one or more target sequences in one or more in vitro samples, comprising: a Cas protein herein; at least one guide polynucleotide comprising a guide sequence designed to have a degree of complementarity with the one or more target sequences, and designed to form a complex with the Cas protein; and an oligonucleotide-based masking construct comprising a non-target sequence, wherein the Cas protein exhibits collateral nuclease activity and cleaves the non-target sequence of the oligo-nucleotide based masking construct once activated by the one or more target sequences.

In another aspect, the present disclosure provides a non-naturally occurring or engineered composition comprising the Cas protein herein that is linked to an inactive first portion of an enzyme or reporter moiety, wherein the enzyme or reporter moiety is reconstituted when contacted with a complementary portion of the enzyme or reporter moiety. In some embodiments, the enzyme or reporter moiety comprises a proteolytic enzyme. In some embodiments, the Cas protein comprises a first Cas protein and a second Cas protein linked to the complementary portion of the enzyme or reporter moiety. In some embodiments, the composition further comprises: i) a first guide capable of forming a complex with the first Cas protein and hybridizing to a first target sequence of a target nucleic acid; and ii) a second guide capable of forming a complex with the second Cas protein, and hybridizing to a second target sequence of the target nucleic acid.

In another aspect, the present disclosure provides a non-naturally occurring or engineered composition comprising one or more polynucleotides encoding the Cas protein and the guide sequence herein.

In another aspect, the present disclosure provides a vector system, which comprises one or more vectors comprising: a first regulatory element operably linked to a nucleotide sequence encoding a Cas protein herein, and a second regulatory element operably linked to a nucleotide sequence encoding the guide sequence. In some embodiments, the nucleotide sequence encoding the Cas protein is codon optimized for expression in a eukaryotic cell. In some embodiments, the vector system is comprised in a single vector. In some embodiments, the one or more vectors comprise viral vectors. In some embodiments, the one or more vectors comprise one or more retroviral, lentiviral, adenoviral, adeno-associated or herpes simplex viral vectors.

In another aspect, the present disclosure provides a delivery system comprising the composition herein, or the system herein, and a delivery vehicle. In some embodiments, the delivery system comprises one or more vectors, or one or more polynucleotide molecules, the one or more vectors or polynucleotide molecules comprising one or more polynucleotide molecules encoding the Cas protein and one or more nucleic acid components of the non-naturally occurring or engineered composition. In some embodiments, the delivery vehicle comprises a ribonucleoprotein complex, one or more particles, one or more vesicles, or one or more viral vectors, liposomes, nanoparticles, exosomes, microvesicles, nucleic acid nanoassemblies, a gene gun, an implantable device, or a vector system. In some embodiments, the one or more particles comprises a lipid, a sugar, a metal or a protein. In some embodiments, the one or more particles comprises lipid nanoparticles. In some embodiments, the one or more vesicles comprises exosomes or liposomes. In some embodiments, the one or more viral vectors comprises one or more adenoviral vectors, one or more lentiviral vectors, or one or more adeno-associated viral vectors.

In another aspect, the present disclosure provides a cell comprising the composition or the system herein. In some embodiments, the cell or progeny thereof is a eukaryotic cell, preferably a human or non-human animal cell, optionally a therapeutic T cell or antibody-producing B-cell or wherein thereof is a eukaryotic the cell is a plant cell.

In another aspect, the present disclosure provides a non-human animal or plant comprising the cell herein, or progeny thereof. In some embodiments, the present disclosure provides the composition herein, or the system herein, or the cell herein, for use in a therapeutic method of treatment.

In another aspect, the present disclosure provides a method of modifying one or more target sequences, the method comprising contacting the one or more target sequences with the composition herein. In some embodiments, modifying the one or more target sequences comprises increasing or decreasing expression of the one or more target sequences. In some embodiments, the system further comprises a recombination template, and wherein modifying the one or more target sequences comprises insertion of the recombination template or a portion thereof. In some embodiments, the one or more target sequences is in a prokaryotic cell. In some embodiments, the one or more target sequences is in a eukaryotic cell.

In another aspect, the present disclosure provides a method of modifying one or more nucleotides in a target sequence, comprising contacting the target sequences with the composition herein. In some embodiments, the target sequence is RNA.

In another aspect, the present disclosure provides a method for detecting a target nucleic acid in a sample comprising: contacting a sample with: the composition herein; and a RNA-based masking construct comprising a non-target sequence; wherein the Cas protein exhibits collateral RNase activity and cleaves the non-target sequence of the detection construct; and detecting a signal from cleavage of the non-target sequence, thereby detecting the target nucleic acid in the sample.

In some embodiments, the method further comprises contacting the sample with reagents for amplifying the target nucleic acid. In some embodiments, the reagents for amplifying comprises isothermal amplification reaction reagents. In some embodiments, the isothermal amplification reagents comprise nucleic-acid sequence-based amplification, recombinase polymerase amplification, loop-mediated isothermal amplification, strand displacement amplification, helicase-dependent amplification, or nicking enzyme amplification reagents. In some embodiments, the target nucleic acid is DNA molecule and the method further comprises contacting the target DNA molecule with a primer comprising an RNA polymerase site and RNA polymerase.

In some embodiments, the masking construct: suppresses generation of a detectable positive signal until the masking construct cleaved or deactivated, or masks a detectable positive signal or generates a detectable negative signal until the masking construct cleaved or deactivated.

In some embodiments, the masking construct comprises: a. a silencing RNA that suppresses generation of a gene product encoded by a reporting construct, wherein the gene product generates the detectable positive signal when expressed; b. a ribozyme that generates the negative detectable signal, and wherein the positive detectable signal is generated when the ribozyme is deactivated; c. a ribozyme that converts a substrate to a first color and wherein the substrate converts to a second color when the ribozyme is deactivated; d. an aptamer and/or comprises a polynucleotide-tethered inhibitor; e. a polynucleotide to which a detectable ligand and a masking component are attached; f. a nanoparticle held in aggregate by bridge molecules, wherein at least a portion of the bridge molecules comprises a polynucleotide, and wherein the solution undergoes a color shift when the nanoparticle is disbursed in solution; g. a quantum dot or fluorophore linked to one or more quencher molecules by a linking molecule, wherein at least a portion of the linking molecule comprises a polynucleotide; h. a polynucleotide in complex with an intercalating agent, wherein the intercalating agent changes absorbance upon cleavage of the polynucleotide; or 1. two fluorophores tethered by a polynucleotide that undergo a shift in fluorescence when released from the polynucleotide.

In some embodiments, the aptamer: a. comprises a polynucleotide-tethered inhibitor that sequesters an enzyme, wherein the enzyme generates a detectable signal upon release from the aptamer or polynucleotide-tethered inhibitor by acting upon a substrate; b. is an inhibitory aptamer that inhibits an enzyme and prevents the enzyme from catalyzing generation of a detectable signal from a substrate or wherein the polynucleotide-tethered inhibitor inhibits an enzyme and prevents the enzyme from catalyzing generation of a detectable signal from a substrate; or c. sequesters a pair of agents that when released from the aptamers combine to generate a detectable signal. In some embodiments, the nanoparticle is a colloidal metal. In some embodiments, the at least one guide polynucleotide comprises a mismatch. In some embodiments, the mismatch is upstream or downstream of a single nucleotide variation on the one or more guide sequences.

In another aspect, the present disclosure provides a method of treating or preventing a disease in a subject, comprising administering the composition, or the system, or the cell herein, to the subject.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A shows protein alignment of five Cas13a sequences with likely thermostability, loci QNRWO1000010.1, OWPA01000389.1, 0153798_10014618, 0153978_10005171, and 0153798_10004687 (SEQ ID NOS: 6026-6031); FIG. 1B shows a Cas13 phylogeny, with identified Cas13a sequences stemming from bioreactors maintained at 55° C. forming a distinct branch in the Cas13a tree.

FIG. 2A QNRWO1000010.1 direct repeat alignment (SEQ ID NOS: 6032-6048); FIG. 2B OWPA01000389.1 direct repeat alignment (SEQ ID NOS: 6049-6054); FIG. 2C 0153798_10014618 direct repeat alignment (SEQ ID NOS: 6055-6058); FIG. 2D 0153978_10005171 direct repeat alignment (SEQ ID NOS: 6059-6062); FIG. 2E 0153798_10004687 direct repeat alignment (SEQ ID NOS: 6063-6066).

FIG. 3A 0153798_10004687 thermophilic Cas13 branch; FIG. 3B 0153978_10005171 thermophilic Cas13 branch; FIG. 3C 0153798_10014618 thermophilic Cas13 branch; FIG. 3D OWPA01000389.1 thermophilic Cas13 branch; FIG. 3E QNRW01000010.1 thermophilic Cas13 branch; FIG. 3F 0J26742_10014101 loci associated with thermophilic Cas 13 branch; and FIG. 3G 0123519_10037894 loci identifying a likely thermostable Cas13a from study conducted at high temperatures.

FIG. 4 shows exemplary methods for identifying novel Cas proteins.

FIG. 5 shows an exemplary method of iterative multi-criterion HMM searches.

FIG. 6 shows an exemplary method of identifying spacer hits to page/bacterial genomes.

FIG. 7 shows an exemplary method of determining estimate feature co-occurrence rates.

FIG. 8 shows hypothesized evolution of various CRISPR systems.

FIG. 9 shows the distribution of sizes of proteins in Cas13 families.

FIG. 10 shows a phylogenetic tree of subgroups of Type VI-B1 Cas proteins.

FIG. 11 shows 6 examples of Cas13b-ts.

FIG. 12 analysis results of CRISPR arrays of Cas13b-t loci.

FIG. 13 shows results of E. coli essential gene screens.

FIG. 14 shows results of E. coli essential gene PFS screens.

FIG. 15 shows 5′ D PFS preferences of exemplary active Cas13b-t orthologs.

FIG. 16 shows depletion of sequences containing PFS by exemplary Cas13b-ts.

FIG. 17 shows gene knockdown mediated by exemplary Cas13b-ts.

FIG. 18 shows knockdown of endogenous transcripts by exemplary Cas13-bts.

FIG. 19 shows A-to-I RNA editing mediated by exemplary Cas13-bts.

FIGS. 20A-20B: FIG. 20A shows the map of the vector expressing targeting guide RNA. FIG. 20B shows the map the vector expressing the non-target guide RNA.

FIG. 21 shows Cas13b-t1, t3 mediated C-to-U editing of reporter transcripts in mammalian cells when fused to evolved CDAR.

FIGS. 22A-22H. Cas13b-t is a functional family of ultra-small Cas nucleases. FIG. 22A. UPGMA dendrogram and protein size distribution of Cas13 subtypes and variants. Previously unknown subfamilies are highlighted. FIG. 22B. Phylogenetic tree of unique Cas13b-t proteins. Points indicate experimentally studied proteins. FIG. 22C. Cas13b-t locus organization. FIG. 22D. CRISPR RNA identified from small RNA sequencing of E. coli containing Cas13b-t2 locus. FIG. 22E. Schematic of PFS placement relative to target sequence. FIG. 22F. E. coli essential gene screen shows Cas13b-t1, 3 and 5 mediate interference with a weak 5′ D (A/G/T) PFS. Weblogos: nucleotides surrounding top 1% of depleted spacers. Histograms: distribution of fold depletion of both targeting and non-targeting spacers. Line plots: relative abundance in final library of spacers targeting regions across normalized positions in the target transcript. FIGS. 22G-22G Evaluation of Cas13b-t1, 3 and 5 for knockdown of (FIG. 22G) luciferase and (FIG. 22H) endogenous transcripts in HEK293FT cells. All values are normalized to a transfection control containing the corresponding gRNA without Cas13b-t expression and are mean+/−standard deviation, n=4. T: targeting gRNA, NT: non-targeting gRNA.

FIGS. 23A-23I. RNA editing with Cas13b-t. FIG. 23A. Schematic of gRNAs mediating RNA editing. Mismatch bubble shown. Mismatch distance refers to the number of nucleotides between the mismatched base and the 5′ end of the DR. FIG. 23B. Evaluation of RNA editing for restoration of a W85X Cypridina luciferase reporter in HEK293FT cells as measured by restoration of luciferase activity. All values are mean+/−standard deviation, n=4 for Cas13b-t1-REPAIR and n=3 for Cas13b-t3-REPAIR. FIGS. 23C-23F. Quantification of RNA editing by Cas13b-t1-REPAIR and RESCUE at indicated target by next-generation sequencing (FIG. 23C) and protein activity assays for selected targets (FIGS. 230D-23F). T: targeting gRNA, NT: Non-targeting gRNA. All values are mean+/−standard deviation, n=4. FIG. 23G. Schematic of directed evolution approach for engineering specific ADAR2dd variants. Selection of both activity and specificity was performed by simultaneous positive selection for editing of a premature stop codon in the ADE2 transcript and negative selection for editing of a premature stop codon in the URA3 transcript. FIG. 23H. Evaluation of specificity-enhancing ADAR2dd mutants applied to Cas13b-t1-REPAIR targeting the W85X (TAG stop codon) Cypridina luciferase reporter as measured by luciferase activity. Restoration of luciferase activity using this reporter with a non-targeting gRNA was used as a proxy for evaluating specificity. FIG. 23I. Quantitative comparison of off-target editing between Cas13b-t1-REPAIR variants. Gold point marks the on-target edit. REPAIR-S refers to addition of E620G and Q696L specificity-enhancing mutants in ADAR2dd. G: Gaussia luciferase transcript, C: Cypridina luciferase transcript.

FIGS. 24A-24B. PFS preferences of Cas13b-t orthologs. FIG. 24A. Workflow of E. coli essential gene screen for determining interference activity and PFS preference of Cas13b-t orthologs. FIG. 24B. Examination of both 5′ and 3′ PFS together reveals that Cas13b-t1, 3 and 5 show preference not only for a 5′ A/T/G, but also a preference for an A in either the +2 or +3 position on the 3′ side. 5′ PFS refers to the single base directly 5′ of the target sequence, and 3′ PFS refers to the +2 and +3 bases on the 3′ side of the target sequence, as the +1 base does not show any preference for any ortholog tested.

FIG. 25. HEPN mutations abolished cleavage activity. Wild-type sequence and sequences with mutation of both the arginine and histidine residues to alanines in both HEPN domains of RanCas13b, Cas13b-t1 and Cas13b-t3 (gray) were targeted to a Gaussia luciferase transcript with two different targeting spacers. Knockdown, as measured by decrease of luciferase activity, was abolished for HEPN-mutated proteins, with RanCas13b acting as a positive control. All values are normalized to a non-targeting spacer condition, with standard error propagation (n=3).

FIGS. 26A-26H. Determination of optimal mismatch distance in RNA editing gRNA spacers. Quantitative evaluation of optimal mismatch distance for (FIGS. 26A 26D) RanCas13b-REPAIR, Cas13b-t1-REPAIR, Cas13b-t3-REPAIR and (FIGS. 26E-26H) RanCas13b-RESCUE, Cas13b-t1-RESCUE, Cas13b-t3-RESCUE targeting the indicated site by next-generation sequencing. In all panels, all values represent mean+/−standard deviation (n=4). Bars represent optimal mismatch distance selected for each target/ortholog for all further experiments. The nucleotide triplet containing the target adenosine or cytosine is shown in parentheses.

FIGS. 27A-27L. Comparison of RNA editing by RanCas13b, Cas13b-t1 and Cas13b-t3 at selected sites. In all panels, all values represent mean+/−standard deviation (n=4). Value for targeting gRNA with REPAIR/RESCUE protein expression condition is shown above the corresponding bar. FIGS. 27A-27I. Measurement of editing rate by next-generation sequencing at indicated target sites. FIG. 27J. Restoration of luciferase activity by A-to-I RNA editing of a W85X Cypridina luciferase reporter. FIG. 27K. Fold activation of beta-catenin by A-to-I RNA editing of the CTNNB1 T41 codon as measured by normalized luciferase activity. FIG. 27L. Restoration of luciferase activity by C-to-U RNA editing of a C82R Gaussia luciferase reporter.

FIGS. 28A-28F. Evaluation of ADAR2dd mutants after Round 1 of evolution. In all panels, all values represent mean+/−standard deviation (n=4). Wt refers to RanCas13b-ADAR2dd(E488Q). All amino acid changes refer to position in ADAR2dd. The nucleotide triplet containing the target adenosine is shown in parentheses. For (FIGS. 28A-28B), Bars or points indicate mutations selected for further analysis. For (FIGS. 28C-28F), the bar or point indicates the final mutation selected from this round of evolution. FIG. 28A. Evaluation of candidate mutants targeting a W13X Cypridina luciferase reporter as measured by restoration of luciferase activity. FIG. 28B. Evaluation of candidate mutants targeting a W85X Cypridina luciferase reporter as measured by restoration of luciferase activity. Nontargeting RLU refers to restoration of luciferase activity in a non-targeting spacer condition and is used as a proxy for off-target editing. FIGS. 28C-28E. Evaluation of selected mutants targeting indicated sites as measured by next generation sequencing. FIG. 28F. Evaluation of candidate mutants targeting a W85X Cypridina luciferase reporter as measured by restoration of luciferase activity. Nontargeting RLU refers to restoration of luciferase activity in a non-targeting spacer condition and is used as a proxy for off-target editing.

FIGS. 29A-29J. Evaluation of ADAR2dd mutants after Round 2 of evolution. In all panels, values represent mean+/−standard deviation (n=4). Wt refers to RanCas13b-ADAR2dd(E488Q) and wt+E620G refers to RanCas13b-ADAR2dd(E488Q/E620G). All amino acid changes refer to position in ADAR2dd and all mutations are on top of an ADAR2dd(E488Q/E620G) background. The nucleotide triplet containing the target adenosine is shown in parentheses. For (FIGS. 29A-29C), bars or points indicate mutations selected for further analysis. For FIGS. 29D-29J, the bar or point indicates the final mutation selected from this round of evolution. FIG. 29A. Evaluation of candidate mutants targeting a R93H Gaussia luciferase reporter as measured by restoration of luciferase activity. FIG. 29B. Evaluation of candidate mutants targeting a W85X (TGA stop codon) Cypridina luciferase reporter as measured by restoration of luciferase activity. FIG. 29C. Evaluation of candidate mutants targeting a W85X (TAG stop codon) Cypridina luciferase reporter as measured by restoration of luciferase activity. Nontargeting RLU refers to restoration of luciferase activity in a non-targeting spacer condition and is used as a proxy for off-target editing. FIGS. 29D-29I. Evaluation of selected candidate mutants targeting indicated sites as measured by next generation sequencing. FIG. 29J. Evaluation of candidate mutants targeting a W85X (TAG stop codon) Cypridina luciferase reporter as measured by restoration of luciferase activity. Nontargeting RLU refers to restoration of luciferase activity in a non-targeting spacer condition and is used as a proxy for off-target editing.

FIGS. 30A-30B. Comparison of off-target edits between REPAIR variants. Quantitative comparison of off-target editing between REPAIR variants in targeting (FIG. 30A) and non-targeting (FIG. 30B) gRNA conditions. Gold point marks the on-target edit. REPAIR-S refers to addition of E620G and Q696L specificity-enhancing mutants in ADAR2dd. G: Gaussia luciferase transcript, C: Cypridina luciferase transcript. Cas13b-t1-REPAIR and REPAIR-S are as shown in FIG. 23I.

FIGS. 31A-31H. Cas13b-tis a functional family of ultra-small Cas nucleases. (FIG. 31A) UPGMA dendrogram and protein size distribution of Cas13 subtypes and variants. Previously unknown subfamilies are highlighted. (FIG. 31B) Phylogenetic tree of unique Cas13b-t proteins. Points indicate experimentally studied proteins. (FIG. 31C) Cas13b-t locus organization. (FIG. 31D) CRISPR RNA identified from small RNA sequencing of E. coli containing Cas13b-t2 locus. (FIG. 31E) Schematic of PFS placement relative to target sequence. (FIG. 31F) E. coli essential gene screen shows Cas13b-t1, 3 and 5 mediate interference with a weak 5′ D (A/G/T) PFS. Weblogos: nucleotides surrounding top 1% of depleted spacers. Histograms: distribution of fold depletion of both targeting and non-targeting spacers. Line plots: relative abundance in final library of spacers targeting regions across normalized positions in the target transcript. (FIGS. 31G-31H) Evaluation of Cas13b-t1, 3 and 5 for knockdown of (FIG. 31G) luciferase and (FIG. 31H) endogenous transcripts in HEK293FT cells. All values are normalized to a transfection control containing the corresponding gRNA without Cas13b-t expression and are mean+/−standard deviation, n=4. T: targeting gRNA, NT: non-targeting gRNA.

FIGS. 32A-32I. RNA editing with Cas13b-t. (FIG. 32A) Schematic of gRNAs mediating RNA editing. Mismatch distance refers to the number of nucleotides between the mismatched base and the 5′ end of the DR. (FIG. 32B) Evaluation of RNA editing for restoration of a W85X Cypridina luciferase reporter in HEK293FT cells as measured by restoration of luciferase activity. All values are mean+/−standard deviation, n=4 for Cas13b-t1-REPAIR and n=3 for Cas13b-t3-REPAIR. (FIGS. 32C-32F) Quantification of RNA editing by Cas13b-t1-REPAIR and RESCUE at indicated target by next-generation sequencing (FIG. 32C) and protein activity assays for selected targets (FIGS. 32D-32F). T: targeting gRNA, NT: Non-targeting gRNA. All values are mean+/−standard deviation, n=4. (FIG. 32G) Schematic of directed evolution approach for engineering specific ADAR2dd variants. Selection of both activity and specificity was performed by simultaneous positive selection for editing of a premature stop codon in the ADE2 transcript and negative selection for editing of a premature stop codon in the URA3 transcript. (FIG. 32H) Evaluation of specificity-enhancing ADAR2dd mutants applied to Cas13b-t1-REPAIR targeting the W85X (TAG stop codon) Cypridina luciferase reporter as measured by luciferase activity. Restoration of luciferase activity using this reporter with a non-targeting gRNA is used as a proxy for evaluating specificity. (FIG. 32I) Quantitative comparison of off-target editing between Cas13b-t1-REPAIR variants. Gold point marks the on-target edit. REPAIR-S refers to addition of E620G and Q696L specificity-enhancing mutants in ADAR2dd. G: Gaussia luciferase transcript, C: Cypridina luciferase transcript.

FIGS. 33A-33B. PFS preferences of Cas13b-t orthologs. (FIG. 33A) Workflow of E. coli essential gene screen for determining interference activity and PFS preference of Cas13b-t orthologs. (FIG. 33B) Examination of both 5′ and 3′ PFS together reveals that Cas13b-t1, 3 and 5 show preference not only for a 5′ A/T/G, but also a preference for an A in either the +2 or +3 position on the 3′ side. 5′ PFS refers to the single base directly 5′ of the target sequence, and 3′ PFS refers to the +2 and +3 bases on the 3′ side of the target sequence, as the +1 base does not show any preference for any ortholog tested.

FIG. 34. HEPN mutations abolish cleavage activity. Wild-type sequence and sequences with mutation of both the arginine and histidine residues to alanines in both HEPN domains of RanCas13b, Cas13b-t1 and Cas13b-t3 were targeted to a Gaussia luciferase transcript with two different targeting spacers. Knockdown, as measured by decrease of luciferase activity, was abolished for HEPN-mutated proteins, with RanCas13b acting as a positive control. All values are normalized to a non-targeting spacer condition, with standard error propagation (n=3).

FIGS. 35A-35H. Determination of optimal mismatch distance in RNA editing gRNA spacers. Quantitative evaluation of optimal mismatch distance for (FIGS. 35A-35D) RanCas13b-REPAIR, Cas13b-t1-REPAIR, Cas13b-t3-REPAIR and (FIGS. 35E-35H) RanCas13b-RESCUE, Cas13b-t1-RESCUE, Cas13b-t3-RESCUE targeting the indicated site by next-generation sequencing. In all panels, all values represent mean+/−standard deviation (n=4). Bars represent optimal mismatch distance selected for each target/ortholog for all further experiments. The nucleotide triplet containing the target adenosine or cytosine is shown in parentheses.

FIGS. 36A-36L. Comparison of RNA editing by RanCas13b, Cas13b-t1 and Cas13b-t3 at selected sites. In all panels, all values represent mean+/−standard deviation (n=4). Value for targeting gRNA with REPAIR/RESCUE protein expression condition is shown above the corresponding bar. (FIGS. 36A-36I) Measurement of editing rate by next-generation sequencing at indicated target sites. (FIG. 36J) Restoration of luciferase activity by A-to-I RNA editing of a W85X Cypridina luciferase reporter. (FIG. 36K) Fold activation of beta-catenin by A-to-I RNA editing of the CTNNB1 T41 codon as measured by normalized luciferase activity. (FIG. 36L) Restoration of luciferase activity by C-to-U RNA editing of a C82R Gaussia luciferase reporter.

FIGS. 37A-37F. Evaluation of ADAR2dd mutants after Round 1 of evolution. In all panels, all values represent mean+/−standard deviation (n=4). Wt refers to RanCas13b-ADAR2dd(E488Q). All amino acid changes refer to position in ADAR2dd. The nucleotide triplet containing the target adenosine is shown in parentheses. For (FIGS. 37A-37B), the bars or points indicate mutations selected for further analysis. For (FIGS. 37C-37F), the bar or point indicates the final mutation selected from this round of evolution. (FIG. 37A). Evaluation of candidate mutants targeting a W13X Cypridina luciferase reporter as measured by restoration of luciferase activity. (FIG. 37B). Evaluation of candidate mutants targeting a W85X Cypridina luciferase reporter as measured by restoration of luciferase activity. Nontargeting RLU refers to restoration of luciferase activity in a non-targeting spacer condition and is used as a proxy for off-target editing. (FIGS. 37C-37E). Evaluation of selected mutants targeting indicated sites as measured by next generation sequencing. (FIG. 37F). Evaluation of candidate mutants targeting a W85X Cypridina luciferase reporter as measured by restoration of luciferase activity. Nontargeting RLU refers to restoration of luciferase activity in a non-targeting spacer condition and is used as a proxy for off-target editing.

FIGS. 38A-38J. Evaluation of ADAR2dd mutants after Round 2 of evolution. In all panels, values represent mean+/−standard deviation (n=4). Wt refers to RanCas13b-ADAR2dd(E488Q) and wt+E620G refers to RanCas13b-ADAR2dd(E488Q/E620G). All amino acid changes refer to position in ADAR2dd and all mutations are on top of an ADAR2dd(E488Q/E620G) background. The nucleotide triplet containing the target adenosine is shown in parentheses. For (FIGS. 38A-38C), bars or points indicate mutations selected for further analysis. For (FIGS. 38D-38J), the bar or point indicates the final mutation selected from this round of evolution. (FIG. 38A). Evaluation of candidate mutants targeting a R93H Gaussia luciferase reporter as measured by restoration of luciferase activity. (FIG. 38B). Evaluation of candidate mutants targeting a W85X (TGA stop codon) Cypridina luciferase reporter as measured by restoration of luciferase activity. (FIG. 38C). Evaluation of candidate mutants targeting a W85X (TAG stop codon) Cypridina luciferase reporter as measured by restoration of luciferase activity. Nontargeting RLU refers to restoration of luciferase activity in a non-targeting spacer condition and is used as a proxy for off-target editing. (FIGS. 38D-38I). Evaluation of selected candidate mutants targeting indicated sites as measured by next generation sequencing. (FIG. 38J). Evaluation of candidate mutants targeting a W85X (TAG stop codon) Cypridina luciferase reporter as measured by restoration of luciferase activity. Nontargeting RLU refers to restoration of luciferase activity in a non-targeting spacer condition and is used as a proxy for off-target editing.

FIGS. 39A-39B. Comparison of off-target edits between REPAIR variants. Quantitative comparison of off-target editing between REPAIR variants in targeting (FIG. 39A) and non-targeting (FIG. 39B) gRNA conditions. Gold point marks the on-target edit. REPAIR-S refers to addition of E620G and Q696L specificity-enhancing mutants in ADAR2dd. G: Gaussia luciferase transcript, C: Cypridina luciferase transcript. Cas13b-t1-REPAIR and REPAIR-S are as shown in FIG. 32I.

FIG. 40—Cas13b-t has collateral activity.

FIG. 41 shows that Cas13b-t-REPAIR mediated RNA editing via AAV delivery of a single AAV vector. (T: Targeting guideRNA; NT: non-targeting guideRNA; GFP: GFP protein delivered instead of REPAIR protein; PBS: no virus control).

The figures herein are for illustrative purposes only and are not necessarily drawn to scale.

### DETAILED DESCRIPTION OF THE EXAMPLE EMBODIMENTS

### Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011)

As used herein, the singular forms “a” “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

A protein or nucleic acid derived from a species means that the protein or nucleic acid has a sequence identical to an endogenous protein or nucleic acid or a portion thereof in the species. The protein or nucleic acid derived from the species may be directly obtained from an organism of the species (e.g., by isolation), or may be produced, e.g., by recombination production or chemical synthesis.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

In one aspect, the present disclosure provides systems and methods for nucleic acid modification. In some examples, the embodiments disclosed herein are directed to non-naturally occurring or engineered systems comprising one or more Cas proteins and one or more guide sequences. The Cas proteins may be engineered to include one or more mutations. In certain embodiments, the engineered Cas protein increases or decreases one or more of protospacer flanking site (PFS) recognition/specificity, gRNA binding, protease activity, polynucleotide binding capability, stability, specificity, target binding, off-target binding, and/or catalytic activity as compared to a corresponding wild-type Cas protein.

In some embodiments, a sub-set of newly identified Cas proteins that are smaller in size than previously discovered Cas proteins, including further modifications to and uses thereof. In some embodiments, the systems comprise one or more Cas proteins that is less than 900 amino acids in size and one or more guide sequences. The relatively small sizes of these Cas protein may allow easier engineering, multiplexing, packaging, and delivery, and being used as a component of a fusion construct, e.g., fusion with a nucleotide deaminase.

In another aspect, the present disclosure provides a base editing system. In some examples, the base editing system comprises a engineered adenosine deaminase comprising (i) E488Q and E620G, (ii) E488Q and Q696L, or (iii) E488Q and V505I, based on amino acid sequence positions of human ADAR2, and corresponding mutations in a homologous ADAR protein. The base editing system may further comprise a dead or nickase form of the Cas13 protein herein associated with (e.g., fused to) the engineered adenosine deaminase.

In another aspect, embodiments disclosed herein include systems and uses for such Cas proteins including diagnostics, base editing therapeutics and methods of detection. Fusion proteins comprising a Cas protein, including those disclosed herein, and nucleotide deaminase may also be used for base editing. Delivery of the proteins and systems disclosed is also provided, including to a variety of cells and via a variety of particles, vesicles and vectors.

### Systems and Compositions in General

In one aspect, the present disclosure provides for systems and compositions for modification of nucleic acids. In general, the systems or composition may comprise one or more Cas protein and one or more guide sequences. In some embodiments, the Cas proteins may be Type VI Cas proteins. The Type VI Cas proteins may be Cas13 proteins. In some examples, the Cas13 proteins may be Cas13a, e.g., SEQ ID NOs. 1-1323. In some examples, the Cas13 proteins may be Cas13b, e.g., SEQ ID NOs. 1324-2770. In some examples, the Cas13 proteins may be Cas13c, e.g., SEQ ID NOs. 2773-2797. In some examples, the Cas13 proteins may be Cas13d, e.g., SEQ ID NOs. 2798-4092. In some examples, the Cas13 proteins may be small Cas13a, e.g., SEQ ID NOs. 4102-4298. In some examples, the Cas13 proteins may be small Cas13b, e.g., SEQ ID NOs. 4299-4654. In some examples, the Cas13 proteins may be small Cas13b-t, e.g., SEQ ID NOs. 2771-2772, 4655-4768, or 5260-5265. In some examples, the Cas13 proteins may be small Cas13c, e.g., SEQ ID NOs. 4769-4797. In some examples, the Cas13 proteins may be small Cas13d, e.g., SEQ ID NOs. 4798-5203.

The Cas13 proteins herein also include variants, homologs, and orthologs of the proteins in SEQ ID NOs. 1-4092, 4102-5203, and 5260-5265.

In some examples, the Cas13 proteins are small proteins, e.g., less than 900 amino acid in size. In some examples, the small Cas13 proteins include Cas13b-t proteins include Cas proteins of a subfamily of Cas13b closely related to the Cas13b ortholog from Alistipes sp. ZOR00009 and is not associated with any auxiliary proteins.

### Crispr-Cas Systems in General

In general, a Cas protein and/or a guide sequence is the component of a CRISPR-Cas system. A CRISPR-Cas system or CRISPR system refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). When the CRISPR protein is a Class 2 Type VI effector, a tracrRNA is not required. In an engineered system of the invention, the direct repeat may encompass naturally-occurring sequences or non-naturally-occurring sequences. The direct repeat of the invention is not limited to naturally occurring lengths and sequences. A direct repeat can be 36 nt in length, but a longer or shorter direct repeat can vary. For example, a direct repeat can be 30 nt or longer, such as 30-100 nt or longer. For example, a direct repeat can be 30 nt, 40 nt, 50 nt, 60 nt, 70 nt, 70 nt, 80 nt, 90 nt, 100 nt or longer in length. In some embodiments, a direct repeat of the invention can include synthetic nucleotide sequences inserted between the 5′ and 3′ ends of naturally occurring direct repeats. In certain embodiments, the inserted sequence may be self-complementary, for example, 20%, 30%, 40%, 50%, 60%, 70%, 80%, 90%, or 100% self-complementary. Furthermore, a direct repeat of the invention may include insertions of nucleotides such as an aptamer or sequences that bind to an adapter protein (for association with functional domains). In certain embodiments, one end of a direct repeat containing such an insertion is roughly the first half of a short DR and the end is roughly the second half of the short DR.

The CRISPR-Cas protein (used interchangeably herein with “Cas protein”, “Cas effector”, “effector”, “effector protein”) may include Cas9, Cas 12 (e.g., Cas12a, Cas12b, Cas12c, Cas12d, etc.), Cas13 (e.g., Cas13a, Cas13b, Cas13b-t, Cas13c, Cas13d, etc.), Cas14, CasX, and CasY. In some embodiments, the CRISPR-Cas protein may be a type VI CRISPR-Cas protein. For example, the Type VI CRISPR-Cas protein may be a Cas13 protein. The Cas13 protein may be Cas13a, Cas13b, Cas 13b-t, Cas13c, or Cas13d. In some examples, the CRISPR-Cas protein is Cas13a. In some examples, the CRISPR-Cas protein is Cas13b. In some examples, the CRISPR-Cas protein is Cas13b-t. In some examples, the CRISPR-Cas protein is Cas13c. In some examples, the CRISPR-Cas protein is Cas13d.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell. In some embodiments, direct repeats may be identified in silico by searching for repetitive motifs that fulfill any or all of the following criteria: 1. found in a 2Kb window of genomic sequence flanking the type II CRISPR locus; 2. span from 20 to 50 bp; and 3. interspaced by 20 to 50 bp. In some embodiments, 2 of these criteria may be used, for instance 1 and 2, 2 and 3, or 1 and 3. In some embodiments, all 3 criteria may be used.

In embodiments of the invention, the terms guide sequence and guide RNA, e.g., RNA capable of guiding CRISPR-Cas effector proteins to a target locus, are used interchangeably as in herein cited documents such as International Patent Publication No. WO 2014/093622 (PCT/US2013/074667). In some embodiments, a guide sequence (or spacer sequence) is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10-40 nucleotides long, such as 20-30 or 20-40 nucleotides long or longer, such as 30 nucleotides long or about 30 nucleotides long. In certain embodiments, the guide sequence is 10-30 nucleotides long, such as 20-30 or 20-40 nucleotides long or longer, such as 30 nucleotides long or about 30 nucleotides long for CRISPR-Cas effectors. In certain embodiments, the guide sequence is 10-30 nucleotides long, such as 20-30 nucleotides long, such as 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some CRISPR-Cas systems, the degree of complementarity between a guide sequence and its corresponding target sequence can be about or more than about 50%, 60%, 75%, 80%, 85%, 90%, 95%, 97.5%, 99%, or 100%; a guide or RNA or crRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or crRNA can be less than about 75, 50, 45, 40, 35, 30, 25, 20, 15, 12, or fewer nucleotides in length; and advantageously tracr RNA is 30 or 50 nucleotides in length. However, an aspect of the invention is to reduce off-target interactions, e.g., reduce the guide interacting with a target sequence having low complementarity. Indeed, in the examples, it is shown that the invention involves mutations that result in the CRISPR-Cas system being able to distinguish between target and off-target sequences that have greater than 80% to about 95% complementarity, e.g., 83%-84% or 88-89% or 94-95% complementarity (for instance, distinguishing between a target having 18 nucleotides from an off-target of 18 nucleotides having 1, 2 or 3 mismatches). Accordingly, in the context of the present invention the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that of f target is 100% or 99.9% or 99.5% or 99% or 99% or 98.5% or 98% or 97.5% or 97% or 96.5% or 96% or 95.5% or 95% or 94.5% complementarity between the sequence and the guide.

In certain embodiments, modulations of cleavage efficiency can be exploited by introduction of mismatches, e.g. 1 or more mismatches, such as 1 or 2 mismatches between spacer sequence and target sequence, including the position of the mismatch along the spacer/target. The more central (e.g., not 3′ or 5′) for instance a double mismatch is, the more cleavage efficiency is affected. Accordingly, by choosing mismatch position along the spacer, cleavage efficiency can be modulated. By means of example, if less than 100% cleavage of targets is desired (e.g. in a cell population), 1 or more, such as preferably 2 mismatches between spacer and target sequence may be introduced in the spacer sequences. The more central along the spacer of the mismatch position, the lower the cleavage percentage.

The methods according to the invention as described herein comprehend inducing one or more nucleotide modifications in a eukaryotic cell (in vitro, i.e. in an isolated eukaryotic cell) as herein discussed comprising delivering to cell a vector as herein discussed. The mutation(s) can include the introduction, deletion, or substitution of one or more nucleotides at each target sequence of cell(s) via the guide(s) RNA(s) or sgRNA(s). The mutations can include the introduction, deletion, or substitution of 1-75 nucleotides at each target sequence of said cell(s) via the guide(s) RNA(s). The mutations can include the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s). The mutations can include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s). The mutations include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s). The mutations can include the introduction, deletion, or substitution of 20, 21, 22, 23, 24, 25, 26, 27, 28, 29, 30, 35, 40, 45, 50, or 75 nucleotides at each target sequence of said cell(s) via the guide(s) RNA(s). The mutations can include the introduction, deletion, or substitution of 40, 45, 50, 75, 100, 200, 300, 400 or 500 nucleotides at each target sequence of said cell(s) via the guide(s) RNA(s).

For minimization of toxicity and off-target effect, it will be important to control the concentration of Cas mRNA or protein and guide RNA delivered. Optimal concentrations of Cas mRNA or protein and guide RNA can be determined by testing different concentrations in a cellular or non-human eukaryote animal model and using deep sequencing the analyze the extent of modification at potential off-target genomic loci.

Typically, in the context of an endogenous CRISPR system, formation of a CRISPR complex (comprising a guide sequence hybridized to a target sequence and complexed with one or more Cas proteins) results in cleavage in or near (e.g. within 1, 2, 3, 4, 5, 6, 7, 8, 9, 10, 20, 50, or more base pairs from) the target sequence, but may depend on for instance secondary structure, in particular in the case of RNA targets. In some cases, in the context of an endogenous CRISPR system, formation of a CRISPR complex (comprising a guide sequence hybridized to a target sequence and complexed with one or more Cas proteins) results in cleavage of one or both strands (if applicable) in or near (e.g. within 1, 2, 3, 4, 5, 6, 7, 8, 9, 10, 20, 50, or more base pairs from) the target sequence.

In particularly preferred embodiments according to the invention, the guide RNA (capable of guiding Cas to a target locus) may comprise (1) a guide sequence capable of hybridizing to a target locus (a polynucleotide target locus, such as an RNA target locus) in the eukaryotic cell; (2) a direct repeat (DR) sequence) which reside in a single RNA, i.e. an sgRNA (arranged in a 5′ to 3′ orientation) or crRNA.

With respect to general information on CRISPR-Cas Systems, components thereof, and delivery of such components, including methods, materials, delivery vehicles, vectors, particles, AAV, and making and using thereof, including as to amounts and formulations, all useful in the practice of the instant invention, reference is made to: U.S. Pat. Nos. 8,999,641, 8,993,233, 8,945,839, 8,932,814, 8,906,616, 8,895,308, 8,889,418, 8,889,356, 8,871,445, 8,865,406, 8,795,965, 8,771,945 and 8,697,359; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958A1 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 A1 (U.S. application Ser. No. 14/183,429); European Patents EP 2 784 162 B1 and EP 2 771 468 B1; European Patent Applications EP 2 771 468 (EP13818570.7), EP 2 764 103 (EP13824232.6), and EP 2 784 162 (EP14170383.5); and PCT Patent Publications PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809).

Reference is also made to U.S. Provisional Application Nos. 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. Provisional Patent Application No. 61/836,123, filed on Jun. 17, 2013. Reference is additionally made to U.S. Provisional Application Nos. 61/835,931, 61/835,936, 61/836,127, 61/836,101, 61/836,080 and 61/835,973, each filed Jun. 17, 2013. Further reference is made to U.S. Provisional Application Nos. 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT Patent applications Nos: PCT/US2014/041803, PCT/US2014/041800, PCT/US2014/041809, PCT/US2014/041804 and PCT/US2014/041806, each filed Jun. 10, 2014 6/10/14; PCT/US2014/041808 filed Jun. 11, 2014; and PCT/US2014/62558 filed Oct. 28, 2014, and U.S. Provisional Application Nos. 61/915,150, 61/915,301, 61/915,267 and 61/915,260, each filed Dec. 12, 2013; 61/757,972 and 61/768,959, filed on Jan. 29, 2013 and Feb. 25, 2013; 61/835,936, 61/836,127, 61/836,101, 61/836,080, 61/835,973, and 61/835,931, filed Jun. 17, 2013; 62/010,888 and 62/010,879, both filed Jun. 11, 2014; 62/010,329 and 62/010,441, each filed Jun. 10, 2014; 61/939,228 and 61/939,242, each filed Feb. 12, 2014; 61/980,012, filed Apr. 15,2014; 62/038,358, filed Aug. 17, 2014; 62/054,490, 62/055,484, 62/055,460 and 62/055,487, each filed Sep. 25, 2014; and 62/069,243, filed Oct. 27, 2014. Reference is also made to U.S. Provisional Application Nos. 62/055,484, 62/055,460, and 62/055,487, filed Sep. 25, 2014; U.S. Provisional Application No. 61/980,012, filed Apr. 15, 2014; and U.S. Provisional Application No. 61/939,242 filed Feb. 12, 2014. Reference is made to PCT application designating, inter alia, the United States, application No. PCT/US14/41806, filed Jun. 10, 2014. Reference is made to U.S. Provisional Application No. 61/930,214 filed on Jan. 22, 2014. Reference is made to U.S. Provisional Application Nos. 61/915,251; 61/915,260 and 61/915,267, each filed on Dec. 12, 2013. Reference is made to U.S. Provisional Application No. 61/980,012 filed Apr. 15, 2014. Reference is made to PCT application designating, inter alia, the United States, application No. PCT/US14/41806, filed Jun. 10, 2014. Reference is made to US Provisional Application Nos. 61/930,214 filed on Jan. 22, 2014. Reference is made to U.S. Provisional Application Nos. 61/915,251; 61/915,260 and 61/915,267, each filed on Dec. 12, 2013.

Mention is also made of U.S. Provisional Application No. 62/091,455, filed 12 Dec. 2014, PROTECTED GUIDE RNAS (PGRNAS); US Provisional Application Nos. 62/096,708, filed 24 Dec. 2014, PROTECTED GUIDE RNAS (PGRNAS); U.S. Provisional Application No. 62/091,462, filed 12 Dec. 2014, DEAD GUIDES FOR CRISPR TRANSCRIPTION FACTORS; U.S. Provisional Application No. 62/096,324, filed 23 Dec. 2014, DEAD GUIDES FOR CRISPR TRANSCRIPTION FACTORS; U.S. Provisional Application No. 62/091,456, filed 12-Dec-14, ESCORTED AND FUNCTIONALIZED GUIDES FOR CRISPR-CAS SYSTEMS; U.S. Provisional Application No. 62/091,461, filed 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. Provisional Application No. 62/094,903, filed 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. Provisional Application No. 62/096,761, filed 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30-Dec-14, RNA-TARGETING SYSTEM; U.S. Provisional Application No. 62/096,656, filed 24 Dec. 2014, CRISPR HAVING OR ASSOCIATED WITH DESTABILIZATION DOMAINS; U.S. Provisional Application No. 62/096,697, filed 24 Dec. 2014, CRISPR HAVING OR ASSOCIATED WITH AAV; U.S. Provisional Application No. 62/098,158, filed 30 Dec. 2014, ENGINEERED CRISPR COMPLEX INSERTIONAL TARGETING SYSTEMS; U.S. Provisional Application No. 62/151,052, filed 22 Apr. 2015, CELLULAR TARGETING FOR EXTRACELLULAR EXOSOMAL REPORTING; U.S. Provisional Application No. 62/054,490, filed 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. Provisional Application No. 62/055,484, filed 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. Provisional Application No. 62/087,537, filed 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. Provisional Application No. 62/054,651, filed 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. Provisional Application No. 62/067,886, filed 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. Provisional Application No. 62/054,675, filed 24-Sep-2014, DELIVERY, USE AND THERAPEUTIC APPLICATIONS OF THE CRISPR-CAS SYSTEMS AND COMPOSITIONS IN NEURONAL CELLS/TISSUES; U.S. Provisional Application No. 62/054,528, filed 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. Provisional Application No. 62/055,454, filed 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. Provisional Application No. 62/055,460, filed 25 Sep. 2014, MULTIFUNCTIONAL-CRISPR COMPLEXES AND/OR OPTIMIZED ENZYME LINKED FUNCTIONAL-CRISPR COMPLEXES; U.S. Provisional Application No. 62/087,475, filed 4 Dec. 2014, FUNCTIONAL SCREENING WITH OPTIMIZED FUNCTIONAL CRISPR-CAS SYSTEMS; U.S. Provisional Application No. 62/055,487, filed 25 Sep. 2014, FUNCTIONAL SCREENING WITH OPTIMIZED FUNCTIONAL CRISPR-CAS SYSTEMS; U.S. Provisional Application No. 62/087,546, filed 4 Dec. 2014, MULTIFUNCTIONAL CRISPR COMPLEXES AND/OR OPTIMIZED ENZYME LINKED FUNCTIONAL-CRISPR COMPLEXES; and U.S. Provisional Application No. 62/098,285, filed 30-Dec-14, CRISPR MEDIATED IN VIVO MODELING AND GENETIC SCREENING OF TUMOR GROWTH AND METASTASIS.

Also with respect to general information on CRISPR-Cas Systems, mention is made of the following (also hereby incorporated herein by reference):
Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013);
RNA-guided editing of bacterial genomes using CRISPR-Cas systems. Jiang W., Bikard D., Cox D., Zhang F, Marraffini L. A. Nat Biotechnol March; 31(3):233-9 (2013);
One-Step Generation of Mice Carrying Mutations in Multiple Genes by CRISPR/Cas-Mediated Genome Engineering. Wang H., Yang H., Shivalila C. S., Dawlaty M M., Cheng A W., Zhang F., Jaenisch R. Cell May 9; 153(4):910-8 (2013);
Optical control of mammalian endogenous transcription and epigenetic states. Konermann S, Brigham M. D., Trevino A. E., Hsu P. D., Heidenreich M, Cong L, Platt R. J., Scott D. A., Church G. M., Zhang F. Nature. August 22; 500(7463):472-6. doi: 10.1038/Nature12466. Epub 2013 Aug. 23 (2013);
Double Nicking by RNA-Guided CRISPR Cas9 for Enhanced Genome Editing Specificity. Ran, F. A., Hsu, P. D., Lin, C. Y., Gootenberg, J. S., Konermann, S., Trevino, A. E., Scott, D. A., Inoue, A., Matoba, S., Zhang, Y., & Zhang, F. Cell August 28. pii: S0092-8674(13)01015-5 (2013-A);
DNA targeting specificity of RNA-guided Cas9 nucleases. Hsu, P., Scott, D., Weinstein, J., Ran, F. A., Konermann, S., Agarwala, V., Li, Y., Fine, E., Wu, X., Shalem, O., Cradick, T. J., Marraffini, L. A., Bao, G., & Zhang, F. Nat Biotechnol doi:10.1038/nbt.2647 (2013);
Genome engineering using the CRISPR-Cas9 system. Ran, F. A., Hsu, P. D., Wright, J., Agarwala, V., Scott, D. A., Zhang, F. Nature Protocols November; 8(11):2281-308 (2013-B);
Genome-Scale CRISPR-Cas9 Knockout Screening in Human Cells. Shalem, O., Sanjana, N. E., Hartenian, E., Shi, X., Scott, D. A., Mikkelson, T., Heckl, D., Ebert, B. L., Root, D. E., Doench, J. G., Zhang, F. Science Dec. 12. (2013). [Epub ahead of print];
Crystal structure of cas9 in complex with guide RNA and target DNA. Nishimasu, H., Ran, F. A., Hsu, P. D., Konermann, S., Shehata, S. I., Dohmae, N., Ishitani, R., Zhang, F., Nureki, O. Cell February 27, 156(5):935-49 (2014);
Genome-wide binding of the CRISPR endonuclease Cas9 in mammalian cells. Wu X., Scott D. A., Kriz A. J., Chiu A. C., Hsu P. D., Dadon D. B., Cheng A. W., Trevino A. E., Konermann S., Chen S., Jaenisch R., Zhang F., Sharp P. A. Nat Biotechnol. April 20. doi: 10.1038/nbt.2889 (2014);
CRISPR-Cas9 Knockin Mice for Genome Editing and Cancer Modeling. Platt R. J., Chen S, Zhou Y, Yim M. J., Swiech L, Kempton H. R., Dahlman J. E., Parnas O, Eisenhaure T. M., Jovanovic M, Graham D. B., Jhunjhunwala S, Heidenreich M, Xavier R. J., Langer R, Anderson D. G., Hacohen N, Regev A, Feng G, Sharp P. A., Zhang F. Cell 159(2): 440-455 DOI: 10.1016/j.cell.2014.09.014(2014);
Development and Applications of CRISPR-Cas9 for Genome Engineering, Hsu P. D., Lander E. S., Zhang F., Cell. June 5; 157(6):1262-78 (2014).
Genetic screens in human cells using the CRISPR/Cas9 system, Wang T, Wei J. J., Sabatini D. M., Lander E. S., Science. January 3; 343(6166): 80-84. doi:10.1126/science.1246981 (2014);
Rational design of highly active sgRNAs for CRISPR-Cas9-mediated gene inactivation, Doench J. G., Hartenian E, Graham D. B., Tothova Z, Hegde M, Smith I, Sullender M, Ebert B L, Xavier R. J., Root D. E., (published online 3 Sep. 2014) Nat Biotechnol. December; 32(12):1262-7 (2014);
In vivo interrogation of gene function in the mammalian brain using CRISPR-Cas9, Swiech L, Heidenreich M, Banerjee A, Habib N, Li Y, Trombetta J, Sur M, Zhang F., (published online 19 Oct. 2014) Nat Biotechnol. January; 33(1):102-6 (2015);
Genome-scale transcriptional activation by an engineered CRISPR-Cas9 complex, Konermann S, Brigham M. D., Trevino A. E., Joung J, Abudayyeh O. O., Barcena C, Hsu P. D., Habib N, Gootenberg J. S., Nishimasu H, Nureki O, Zhang F., Nature. January 29; 517(7536):583-8 (2015).
A split-Cas9 architecture for inducible genome editing and transcription modulation, Zetsche B, Volz S E, Zhang F., (published online 2 Feb. 2015) Nat Biotechnol. February; 33(2):139-42 (2015);
Genome-wide CRISPR Screen in a Mouse Model of Tumor Growth and Metastasis, Chen S, Sanjana N. E., Zheng K, Shalem O, Lee K, Shi X, Scott D. A., Song J, Pan J. Q., Weissleder R, Lee H, Zhang F, Sharp P. A. Cell 160, 1246-1260, Mar. 12, 2015 (multiplex screen in mouse), and
In vivo genome editing using Staphylococcus aureus Cas9, Ran F A, Cong L, Yan W. X., Scott D. A., Gootenberg J. S., Kriz A. J., Zetsche B, Shalem O, Wu X, Makarova K S, Koonin E. V., Sharp P. A., Zhang F., (published online 1 Apr. 2015), Nature. April 9; 520(7546):186-91(2015).
Shalem et al., “High-throughput functional genomics using CRISPR-Cas9,” Nature Reviews Genetics 16, 299-311 (May 2015).
Xu et al., “Sequence determinants of improved CRISPR sgRNA design,” Genome Research 25, 1147-1157 (August 2015).
Parnas et al., “A Genome-wide CRISPR Screen in Primary Immune Cells to Dissect Regulatory Networks,” Cell 162, 675-686 (Jul. 30, 2015).
Ramanan et al., CRISPR/Cas9 cleavage of viral DNA efficiently suppresses hepatitis B virus,” Scientific Reports 5:10833. doi: 10.1038/srep10833 (Jun. 2, 2015)
Nishimasu et al., Crystal Structure of Staphylococcus aureus Cas9,” Cell 162, 1113-1126 (Aug. 27, 2015)
Zetsche et al. (2015), “Cpf1 is a single RNA-guided endonuclease of a class 2 CRISPR-Cas system,” Cell 163, 759-771 (Oct. 22, 2015) doi: 10.1016/j.cell.2015.09.038. Epub Sep. 25, 2015
Shmakov et al. (2015), “Discovery and Functional Characterization of Diverse Class 2 CRISPR-Cas Systems,” Molecular Cell 60, 385-397 (Nov. 5, 2015) doi: 10.1016/j.molcel.2015.10.008. Epub Oct. 22, 2015
Dahlman et al., “Orthogonal gene control with a catalytically active Cas9 nuclease,” Nature Biotechnology 33, 1159-1161 (November, 2015)
Gao et al, “Engineered Cpf1 Enzymes with Altered PAM Specificities,” bioRxiv 091611; doi: dx.doi.org/10.1101/091611 Epub Dec. 4, 2016
Smargon et al. (2017), “Cas13b Is a Type VI-B CRISPR-Associated RNA-Guided RNase Differentially Regulated by Accessory Proteins Csx27 and Csx28,” Molecular Cell 65, 618-630 (Feb. 16, 2017) doi: 10.1016/j.molcel.2016.12.023. Epub Jan. 5, 2017 each of which is incorporated herein by reference, may be considered in the practice of the instant invention, and discussed briefly below:
Cong et al. engineered type II CRISPR-Cas systems for use in eukaryotic cells based on both Streptococcus thermophilus Cas9 and also Streptococcus pyogenes Cas9 and demonstrated that Cas9 nucleases can be directed by short RNAs to induce precise cleavage of DNA in human and mouse cells. Their study further showed that Cas9 as converted into a nicking enzyme can be used to facilitate homology-directed repair in eukaryotic cells with minimal mutagenic activity. Additionally, their study demonstrated that multiple guide sequences can be encoded into a single CRISPR array to enable simultaneous editing of several at endogenous genomic loci sites within the mammalian genome, demonstrating easy programmability and wide applicability of the RNA-guided nuclease technology. This ability to use RNA to program sequence specific DNA cleavage in cells defined a new class of genome engineering tools. These studies further showed that other CRISPR loci are likely to be transplantable into mammalian cells and can also mediate mammalian genome cleavage. Importantly, it can be envisaged that several aspects of the CRISPR-Cas system can be further improved to increase its efficiency and versatility.
Jiang et al. used the clustered, regularly interspaced, short palindromic repeats (CRISPR)-associated Cas9 endonuclease complexed with dual-RNAs to introduce precise mutations in the genomes of Streptococcus pneumoniae and Escherichia coli. The approach relied on dual-RNA:Cas9-directed cleavage at the targeted genomic site to kill unmutated cells and circumvents the need for selectable markers or counter-selection systems. The study reported reprogramming dual-RNA:Cas9 specificity by changing the sequence of short CRISPR RNA (crRNA) to make single- and multinucleotide changes carried on editing templates. The study showed that simultaneous use of two crRNAs enabled multiplex mutagenesis. Furthermore, when the approach was used in combination with recombineering, in S. pneumoniae, nearly 100% of cells that were recovered using the described approach contained the desired mutation, and in E. coli, 65% that were recovered contained the mutation.
Wang et al. (2013) used the CRISPR/Cas system for the one-step generation of mice carrying mutations in multiple genes which were traditionally generated in multiple steps by sequential recombination in embryonic stem cells and/or time-consuming intercrossing of mice with a single mutation. The CRISPR/Cas system will greatly accelerate the in vivo study of functionally redundant genes and of epistatic gene interactions.
Konermann et al. (2013) addressed the need in the art for versatile and robust technologies that enable optical and chemical modulation of DNA-binding domains based CRISPR Cas9 enzyme and also Transcriptional Activator Like Effectors
Ran et al. (2013-A) described an approach that combined a Cas9 nickase mutant with paired guide RNAs to introduce targeted double-strand breaks. This addresses the issue of the Cas9 nuclease from the microbial CRISPR-Cas system being targeted to specific genomic loci by a guide sequence, which can tolerate certain mismatches to the DNA target and thereby promote undesired off-target mutagenesis. Because individual nicks in the genome are repaired with high fidelity, simultaneous nicking via appropriately of fset guide RNAs is required for double-stranded breaks and extends the number of specifically recognized bases for target cleavage. The authors demonstrated that using paired nicking can reduce off-target activity by 50- to 1,500-fold in cell lines and to facilitate gene knockout in mouse zygotes without sacrificing on-target cleavage efficiency. This versatile strategy enables a wide variety of genome editing applications that require high specificity.
Hsu et al. (2013) characterized SpCas9 targeting specificity in human cells to inform the selection of target sites and avoid off-target effects. The study evaluated >700 guide RNA variants and SpCas9-induced indel mutation levels at >100 predicted genomic off-target loci in 293T and 293FT cells. The authors that SpCas9 tolerates mismatches between guide RNA and target DNA at different positions in a sequence-dependent manner, sensitive to the number, position and distribution of mismatches. The authors further showed that SpCas9-mediated cleavage is unaffected by DNA methylation and that the dosage of SpCas9 and sgRNA can be titrated to minimize off-target modification. Additionally, to facilitate mammalian genome engineering applications, the authors reported providing a web-based software tool to guide the selection and validation of target sequences as well as off-target analyses.
Ran et al. (2013-B) described a set of tools for Cas9-mediated genome editing via non-homologous end joining (NHEJ) or homology-directed repair (HDR) in mammalian cells, as well as generation of modified cell lines for downstream functional studies. To minimize off-target cleavage, the authors further described a double-nicking strategy using the Cas9 nickase mutant with paired guide RNAs. The protocol provided by the authors experimentally derived guidelines for the selection of target sites, evaluation of cleavage efficiency and analysis of off-target activity. The studies showed that beginning with target design, gene modifications can be achieved within as little as 1-2 weeks, and modified clonal cell lines can be derived within 2-3 weeks.
Shalem et al. described a new way to interrogate gene function on a genome-wide scale. Their studies showed that delivery of a genome-scale CRISPR-Cas9 knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hits NF2, CUL3, TADA2B, and TADA1. The authors observed a high level of consistency between independent guide RNAs targeting the same gene and a high rate of hit confirmation, and thus demonstrated the promise of genome-scale screening with Cas9.
Nishimasu et al. reported the crystal structure of Streptococcus pyogenes Cas9 in complex with sgRNA and its target DNA at 2.5 A° resolution. The structure revealed a bilobed architecture composed of target recognition and nuclease lobes, accommodating the sgRNA:DNA heteroduplex in a positively charged groove at their interface. Whereas the recognition lobe is essential for binding sgRNA and DNA, the nuclease lobe contains the HNH and RuvC nuclease domains, which are properly positioned for cleavage of the complementary and non-complementary strands of the target DNA, respectively. The nuclease lobe also contains a carboxyl-terminal domain responsible for the interaction with the protospacer adjacent motif (PAM). This high-resolution structure and accompanying functional analyses have revealed the molecular mechanism of RNA-guided DNA targeting by Cas9, thus paving the way for the rational design of new, versatile genome-editing technologies.
Wu et al. mapped genome-wide binding sites of a catalytically inactive Cas9 (dCas9) from Streptococcus pyogenes loaded with single guide RNAs (sgRNAs) in mouse embryonic stem cells (mESCs). The authors showed that each of the four sgRNAs tested targets dCas9 to between tens and thousands of genomic sites, frequently characterized by a 5-nucleotide seed region in the sgRNA and an NGG protospacer adjacent motif (PAM). Chromatin inaccessibility decreases dCas9 binding to other sites with matching seed sequences; thus 70% of off-target sites are associated with genes. The authors showed that targeted sequencing of 295 dCas9 binding sites in mESCs transfected with catalytically active Cas9 identified only one site mutated above background levels. The authors proposed a two-state model for Cas9 binding and cleavage, in which a seed match triggers binding but extensive pairing with target DNA is required for cleavage.
Platt et al. established a Cre-dependent Cas9 knockin mouse. The authors demonstrated in vivo as well as ex vivo genome editing using adeno-associated virus (AAV)-, lentivirus-, or particle-mediated delivery of guide RNA in neurons, immune cells, and endothelial cells.
Hsu et al. (2014) is a review article that discusses generally CRISPR-Cas9 history from yogurt to genome editing, including genetic screening of cells.
Wang et al. (2014) relates to a pooled, loss-of-function genetic screening approach suitable for both positive and negative selection that uses a genome-scale lentiviral single guide RNA (sgRNA) library.
Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.
Swiech et al. demonstrate that AAV-mediated SpCas9 genome editing can enable reverse genetic studies of gene function in the brain.
Konermann et al. (2015) discusses the ability to attach multiple effector domains, e.g., transcriptional activator, functional and epigenomic regulators at appropriate positions on the guide such as stem or tetraloop with and without linkers.
Zetsche et al. demonstrates that the Cas9 enzyme can be split into two and hence the assembly of Cas9 for activation can be controlled.
Chen et al. relates to multiplex screening by demonstrating that a genome-wide in vivo CRISPR-Cas9 screen in mice reveals genes regulating lung metastasis.
Ran et al. (2015) relates to SaCas9 and its ability to edit genomes and demonstrates that one cannot extrapolate from biochemical assays. Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Shalem et al. (2015) described ways in which catalytically inactive Cas9 (dCas9) fusions are used to synthetically repress (CRISPRi) or activate (CRISPRa) expression, showing. advances using Cas9 for genome-scale screens, including arrayed and pooled screens, knockout approaches that inactivate genomic loci and strategies that modulate transcriptional activity.
Xu et al. (2015) assessed the DNA sequence features that contribute to single guide RNA (sgRNA) efficiency in CRISPR-based screens. The authors explored efficiency of CRISPR/Cas9 knockout and nucleotide preference at the cleavage site. The authors also found that the sequence preference for CRISPRi/a is substantially different from that for CRISPR/Cas9 knockout.
Parnas et al. (2015) introduced genome-wide pooled CRISPR-Cas9 libraries into dendritic cells (DCs) to identify genes that control the induction of tumor necrosis factor (Tnf) by bacterial lipopolysaccharide (LPS). Known regulators of Tlr4 signaling and previously unknown candidates were identified and classified into three functional modules with distinct effects on the canonical responses to LPS.
Ramanan et al (2015) demonstrated cleavage of viral episomal DNA (cccDNA) in infected cells. The HBV genome exists in the nuclei of infected hepatocytes as a 3.2 kb double-stranded episomal DNA species called covalently closed circular DNA (cccDNA), which is a key component in the HBV life cycle whose replication is not inhibited by current therapies. The authors showed that sgRNAs specifically targeting highly conserved regions of HBV robustly suppresses viral replication and depleted cccDNA.
Nishimasu et al. (2015) reported the crystal structures of SaCas9 in complex with a single guide RNA (sgRNA) and its double-stranded DNA targets, containing the 5′-TTGAAT-3′ PAM and the 5′-TTGGGT-3′ PAM. A structural comparison of SaCas9 with SpCas9 highlighted both structural conservation and divergence, explaining their distinct PAM specificities and orthologous sgRNA recognition.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells. In addition, mention is made of PCT application PCT/US14/70057, Attorney Reference 47627.99.2060 and BI-2013/107 entitled “DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS (claiming priority from one or more or all of US provisional patent applications: 62/054,490, filed Sep. 24, 2014; 62/010,441, filed Jun. 10, 2014; and 61/915,118, 61/915,215 and 61/915,148, each filed on Dec. 12, 2013) (“the Particle Delivery PCT”), incorporated herein by reference, with respect to a method of preparing an sgRNA-and-Cas9 protein containing particle comprising admixing a mixture comprising an sgRNA and Cas9 protein (and optionally HDR template) with a mixture comprising or consisting essentially of or consisting of surfactant, phospholipid, biodegradable polymer, lipoprotein and alcohol; and particles from such a process. For example, wherein Cas9 protein and sgRNA were mixed together at a suitable, e.g., 3:1 to 1:3 or 2:1 to 1:2 or 1:1 molar ratio, at a suitable temperature, e.g., 15-30C, e.g., 20-25C, e.g., room temperature, for a suitable time, e.g., 15-45, such as 30 minutes, advantageously in sterile, nuclease free buffer, e.g., 1×PBS. Separately, particle components such as or comprising: a surfactant, e.g., cationic lipid, e.g., 1,2-dioleoyl-3-trimethylammonium-propane (DOTAP); phospholipid, e.g., dimyristoylphosphatidylcholine (DMPC); biodegradable polymer, such as an ethylene-glycol polymer or PEG, and a lipoprotein, such as a low-density lipoprotein, e.g., cholesterol were dissolved in an alcohol, advantageously a C₁₋₆ alkyl alcohol, such as methanol, ethanol, isopropanol, e.g., 100% ethanol. The two solutions were mixed together to form particles containing the Cas9-sgRNA complexes. Accordingly, sgRNA may be pre-complexed with the Cas9 protein, before formulating the entire complex in a particle. Formulations may be made with a different molar ratio of different components known to promote delivery of nucleic acids into cells (e.g. 1,2-dioleoyl-3-trimethylammonium-propane (DOTAP), 1,2-ditetradecanoyl-sn-glycero-3-phosphocholine (DMPC), polyethylene glycol (PEG), and cholesterol) For example DOTAP:DMPC:PEG:Cholesterol Molar Ratios may be DOTAP 100, DMPC 0, PEG 0, Cholesterol 0; or DOTAP 90, DMPC 0, PEG 10, Cholesterol 0; or DOTAP 90, DMPC 0, PEG 5, Cholesterol 5. DOTAP 100, DMPC 0, PEG 0, Cholesterol 0. That application accordingly comprehends admixing sgRNA, Cas9 protein and components that form a particle; as well as particles from such admixing. Aspects of the instant invention can involve particles; for example, particles using a process analogous to that of the Particle Delivery PCT, e.g., by admixing a mixture comprising crRNA and/or CRISPR-Cas as in the instant invention and components that form a particle, e.g., as in the Particle Delivery PCT, to form a particle and particles from such admixing (or, of course, other particles involving crRNA and/or CRISPR-Cas as in the instant invention).

### Multiplex Targeting Approach

The Cas proteins herein can employ more than one guide molecules without losing activity. This may enable the use of the Cas proteins, CRISPR-Cas systems or complexes as defined herein for targeting multiple targets (e.g., DNA targets), genes or gene loci, with a single enzyme, system or complex as defined herein. The guide molecules may be tandemly arranged, optionally separated by a nucleotide sequence such as a direct repeat as defined herein. The position of the different guide molecules is the tandem does not influence the activity.

In any of the described methods the complex may be delivered with multiple guides for multiplexed use. In any of the described methods more than one protein(s) may be used. In some examples, one Cas protein may be delivered with multiple guides, e.g., at least 2, at least 5, at least 10, at least 15, at least 20, at least 30, at least 40, at least 50, at least 60, at least 70, at least 80, at least 90, at least 100, at least 120, at least 140, at least 160, at least 180, at least 200, at least 220, at least 240, at least 260, at least 280, at least 300, at least 350, at least 400, or at least 500 guides. In some examples, a system herein may comprise a Cas protein and multiple guides, e.g., at least 2, at least 5, at least 10, at least 15, at least 20, at least 30, at least 40, at least 50, at least 60, at least 70, at least 80, at least 90, at least 100, at least 120, at least 140, at least 160, at least 180, at least 200, at least 220, at least 240, at least 260, at least 280, at least 300, at least 350, at least 400, or at least 500 guides.

The Cas protein may form part of a CRISPR system or complex, which further comprises tandemly arranged guide RNAs (gRNAs) comprising a series of 2, 3, 4, 5, 6, 7, 8, 9, 10, 15, 25, 25, 30, or more than 30 guide sequences, each capable of specifically hybridizing to a target sequence in a genomic locus of interest in a cell. In some embodiments, the functional Cas CRISPR system or complex binds to the multiple target sequences. In some embodiments, the functional CRISPR system or complex may edit the multiple target sequences, e.g., the target sequences may comprise a genomic locus, and in some embodiments, there may be an alteration of gene expression. In some embodiments, the functional CRISPR system or complex may comprise further functional domains. In some embodiments, the composition comprises two or more guide sequences capable of hybridizing to two different target sequences or different regions of a target sequence.

In some embodiments, the invention provides a method for altering or modifying expression of multiple gene products. The method may comprise introducing into a cell containing said target nucleic acids, e.g., DNA molecules, or containing and expressing target nucleic acid, e.g., DNA molecules; for instance, the target nucleic acids may encode gene products or provide for expression of gene products (e.g., regulatory sequences). In some general embodiments, the Cas enzyme used for multiplex targeting is associated with one or more functional domains. In some more specific embodiments, the CRISPR enzyme used for multiplex targeting is a deadCas as defined herein elsewhere. In some embodiments, each of the guide sequence is at least 16, 17, 18, 19, 20, 25 nucleotides, or between 16-30, or between 16-25, or between 16-20 nucleotides in length. Examples of multiplex genome engineering using CRISPR effector proteins are provided in Cong et al. (Science February 15; 339(6121):819-23 (2013) and other publications cited herein.

In any of the described methods the strand break may be a single strand break or a double strand break. In preferred embodiments the double strand break may refer to the breakage of two sections of RNA, such as the two sections of RNA formed when a single strand RNA molecule has folded onto itself or putative double helices that are formed with an RNA molecule which contains self-complementary sequences allows parts of the RNA to fold and pair with itself.

Provided herein are engineered polynucleotide sequences that can direct the activity of a CRISPR protein to multiple targets using a single crRNA. The engineered polynucleotide sequences, also referred to as multiplexing polynucleotides, can include two or more direct repeats interspersed with two or more guide sequences. More specifically, the engineered polynucleotide sequences can include a direct repeat sequence having one or more mutations relative to the corresponding wild type direct repeat sequence. The engineered polynucleotide can be configured, for example, as: 5′ DR1-G1-DR2-G2 3′. In some embodiments, the engineered polynucleotide can be configured to include three, four, five, or more additional direct repeat and guide sequences, for example: 5′ DR1-G1-DR2-G2-DR3-G3 3′, 5″ DR1-G1-DR2-G2-DR3-G3-DR4-G4 3′, or 5′ DR1-G1-DR2-G2-DR3-G3-DR4-G4-DR5-G5 3′.

Regardless of the number of direct repeat sequences, the direct repeat sequences differ from one another. Thus, DR1 can be a wild type sequence and DR2 can include one or more mutations relative to the wild type sequence in accordance with the disclosure provided herein regarding direct repeats for Cas orthologs. The guide sequences can also be the same or different. In some embodiments, the guide sequences can bind to different nucleic acid targets, for example, nucleic acids encoding different polypeptides. The multiplexing polynucleotides can be as described, for example, at [0039]—[0072] in U.S. Application 62/780,748 entitled “CRISPR Cpf1 Direct Repeat Variants” and filed Dec. 17, 2018, incorporated herein in its entirety by reference.

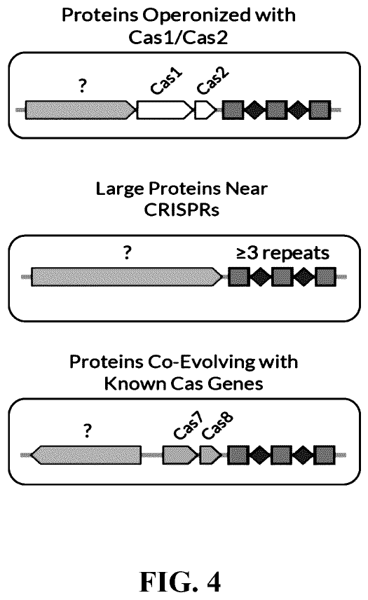

Multiplex design of guide molecules for the detection of coronaviruses and/or other respiratory viruses in a sample to identify the cause of a respiratory infection is envisioned, and design can be according to the methods disclosed herein. Briefly, the design of guide molecules can encompass utilization of training models described herein using a variety of input features, which may include the particular Cas protein used for targeting of the sequences of interest. See U.S. Provisional Application 62/818,702 FIG. 4A, incorporated specifically by reference. Guide molecules can be designed as detailed elsewhere herein. Regarding detection of coronavirus, guide design can be predicated on genome sequences disclosed in Tian et al, “Potent binding of 2019 novel coronavirus spike protein by a SARS coronavirus-specific human monoclonal antibody”; doi: 10.1101/2020.01.28.923011, incorporated by reference, which details human monoclonal antibody, CR3022 binding of the 2019-nCoV RBD (KD of 6.3 nM) or Sequences of the 2019-nCoV are available at GISAID accession no. EPI_ISL_402124 and EPI_ISL_402127-402130, and described in doi:10.1101/2020.01.22.914952, or EP_ISL_402119-402121 and EP_ISL_402123-402124; see also GenBank Accession No. MN908947.3. Guide design can target unique viral genomic regions of the 2019-nCoV or conserved genomic regions across one or more viruses of the coronavirus family.

### Type VI Cas Proteins

In some embodiments, the Cas proteins herein are Class 2 Type VI Cas proteins. Type VI Cas proteins include Cas proteins that contain one or more (e.g., two) higher eukaryotes and prokaryotes nucleotide-binding (HEPN) domains. HEPN domains are common in various defense systems, the experimentally characterized of which, such as the toxins of numerous prokaryotic toxin-antitoxin systems or eukaryotic RNase L, all have RNase activity. Examples of HEPN include those described in Anantharaman V, Makarova K. S., Burroughs A. M., Koonin E. V., Aravind L. Comprehensive analysis of the HEPN superfamily: identification of novel roles in intra-genomic conflicts. Examples of Type VI Cas proteins include those described in Shmakov S, et al. Discovery and functional characterization of diverse class 2 CRISPR-Cas systems. Mol. Cell. 2015; 60:385-397, Shmakov S, et al. Nat Rev Microbiol. 2017 March; 15(3): 169-182; and Makarova, K. S., Wolf, Y. I., Iranzo, J. et al. Evolutionary classification of CRISPR-Cas systems: a burst of class 2 and derived variants. Nat Rev Microbiol 18, 67-83 (2020), which are incorporated by reference herein in their entireties.

In an embodiment, a HEPN domain comprises at least one RxxxxH motif comprising the sequence of R{N/H/K}X₁X₂X₃H. In an embodiment of the invention, a HEPN domain comprises a RxxxxH motif comprising the sequence of R{N/H}X₁X₂X₃H. In an embodiment of the invention, a HEPN domain comprises the sequence of R{N/K}X₁X₂X₃H. In certain embodiments, X₁ is R, S, D, E, Q, N, G, Y, or H. In certain embodiments, X₂ is I, S, T, V, or L. In certain embodiments, X₃ is L, F, N, Y, V, I, S, D, E, or A.

In some embodiments, the systems or compositions comprise a protein comprising one or more HEPN domains and is less than 1000 amino acids in length. For example, the protein may be less than 950, less than 900, less than 850, less than 800, less than 750, less than 700, less than 650, less than 600, less than 550, or less than 500 amino acids in size.

### Cas13 in General

In some examples, the Type VI Cas proteins are Cas13 proteins. Examples of Cas 13 proteins include Cas13a, Cas13b, Cas13c, Cas13d, and Cas13b-t. The instant invention provides particular Cas13 effectors, nucleic acids, systems, vectors, and methods of use. The features and functions of Cas13 may also be the features and functions of other CRISPR-Cas proteins described herein. In some examples, the CRISPR-Cas protein is Cas13a. In some examples, the CRISPR-Cas protein is Cas13b. In some examples, the CRISPR-Cas protein is Cas13b-t. In some examples, the CRISPR-Cas protein is Cas13c. In some examples, the CRISPR-Cas protein is Cas13d.

Cas13 proteins may have RNA binding and cleaving function. In particular embodiments, the Cas13 proteins may have RNA and/or DNA cleaving function, e.g., RNA cleaving function. The systems and methods herein may be used to introduce one or more mutations in nucleic acids. The mutation(s) can include the introduction, deletion, or substitution of one or more nucleotides at each target sequence of cell(s) via the guide(s) RNA(s) or sgRNA(s) or crRNA(s). The mutations can include the introduction, deletion, or substitution of 1-75 nucleotides at each target sequence of said cell(s) via the guide(s) RNA(s) or sgRNA(s) or crRNA(s). The mutations can include the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s) or crRNA(s). The mutations can include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s) or crRNA(s). The mutations include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s) via the guide(s) RNA(s) or sgRNA(s) or crRNA(s). The mutations can include the introduction, deletion, or substitution of 20, 21, 22, 23, 24, 25, 26, 27, 28, 29, 30, 35, 40, 45, 50, or 75 nucleotides at each target sequence of said cell(s) via the guide(s) RNA(s) or sgRNA(s) or crRNA(s). The mutations can include the introduction, deletion, or substitution of 40, 45, 50, 75, 100, 200, 300, 400 or 500 nucleotides at each target sequence of said cell(s) via the guide(s) RNA(s) or sgRNA(s) or crRNAs.

For minimization of toxicity and off-target effect, it will be important to control the concentration of Cas13 mRNA and guide RNA delivered. Optimal concentrations of Cas13 mRNA and guide RNA can be determined by testing different concentrations in a cellular or non-human eukaryote animal model and using deep sequencing the analyze the extent of modification at potential off-target genomic loci. Guide sequences and strategies to minimize toxicity and off-target effects can be as in WO 2014/093622 (PCT/US2013/074667); or, via mutation as herein.

In some embodiments, the Cas proteins may have cleavage activity. In some embodiments, Cas13 may direct cleavage of one or two nucleic acid strands at the location of or near a target sequence, such as within the target sequence and/or within the complement of the target sequence or at sequences associated with the target sequence, e.g., within about 1, 2, 3, 4, 5, 6, 7, 8, 9, 10, 15, 20, 25, 50, 100, 200, 500, or more base pairs from the first or last nucleotide of a target sequence. In some embodiments, the Cas13 protein may direct more than one cleavage (such as one, two three, four, five, or more cleavages) of one or two strands within the target sequence and/or within the complement of the target sequence or at sequences associated with the target sequence and/or within about 1, 2, 3, 4, 5, 6, 7, 8, 9, 10, 15, 20, 25, 50, 100, 200, 500, or more base pairs from the first or last nucleotide of a target sequence. In some embodiments, the cleavage may be blunt, i.e., generating blunt ends. In some embodiments, the cleavage may be staggered, i.e., generating sticky ends. In some embodiments, a vector encodes a nucleic acid-targeting Cas13 protein that may be mutated with respect to a corresponding wild-type enzyme such that the mutated nucleic acid-targeting Cas13 protein lacks the ability to cleave one or two strands of a target polynucleotide containing a target sequence, e.g., alteration or mutation in a HEPN domain to produce a mutated Cas13 substantially lacking all RNA cleavage activity, e.g., the RNA cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form. By derived, Applicants mean that the derived enzyme is largely based, in the sense of having a high degree of sequence homology with, a wildtype enzyme, but that it has been mutated (modified) in some way as known in the art or as described herein.

Typically, in the context of an endogenous RNA-targeting system, formation of a RNA-targeting complex (comprising a guide RNA or crRNA hybridized to a target sequence and complexed with one or more RNA-targeting effector proteins) results in cleavage of RNA strand(s) in or near (e.g., within 1, 2, 3, 4, 5, 6, 7, 8, 9, 10, 20, 50, or more base pairs from) the target sequence. As used herein the term “sequence(s) associated with a target locus of interest” refers to sequences near the vicinity of the target sequence (e.g. within 1, 2, 3, 4, 5, 6, 7, 8, 9, 10, 20, 50, or more base pairs from the target sequence, wherein the target sequence is comprised within a target locus of interest).

The (i) Cas13 or nucleic acid molecule(s) encoding it or (ii) crRNA can be delivered separately; and advantageously at least one or both of one of (i) and (ii), e.g., an assembled complex is delivered via a particle or nanoparticle complex. RNA-targeting effector protein mRNA can be delivered prior to the RNA-targeting guide RNA or crRNA to give time for nucleic acid-targeting effector protein to be expressed. RNA-targeting effector protein (Cas13) mRNA might be administered 1-12 hours (preferably around 2-6 hours) prior to the administration of RNA-targeting guide RNA or crRNA. Alternatively, RNA-targeting effector protein mRNA and RNA-targeting guide RNA or crRNA can be administered together. Advantageously, a second booster dose of guide RNA or crRNA can be administered 1-12 hours (preferably around 2-6 hours) after the initial administration of RNA-targeting effector (Cas13) protein mRNA+guide RNA. Additional administrations of RNA-targeting effector protein mRNA and/or guide RNA or crRNA might be useful to achieve the most efficient levels of genome modification.

In one embodiment, the systems and methods herein may be used for cleaving a target RNA. The method may comprise modifying a target RNA using a RNA-targeting complex that binds to the target RNA and effect cleavage of said target RNA. In an embodiment, the systems or compositions herein, when introduced into a cell, may create a break (e.g., a single or a double strand break) in the RNA sequence. For example, the systems and methods can be used to cleave a disease RNA in a cell. For example, an exogenous RNA template comprising a sequence to be integrated flanked by an upstream sequence and a downstream sequence may be introduced into a cell. The upstream and downstream sequences share sequence similarity with either side of the site of integration in the RNA. Where desired, a donor RNA can be mRNA. The exogenous RNA template comprises a sequence to be integrated (e.g., a mutated RNA). The sequence for integration may be a sequence endogenous or exogenous to the cell. Examples of a sequence to be integrated include RNA encoding a protein or a non-coding RNA (e.g., a microRNA). Thus, the sequence for integration may be operably linked to an appropriate control sequence or sequences. Alternatively, the sequence to be integrated may provide a regulatory function. The upstream and downstream sequences in the exogenous RNA template are selected to promote recombination between the RNA sequence of interest and the donor RNA. The upstream sequence may be a RNA sequence that shares sequence similarity with the RNA sequence upstream of the targeted site for integration. Similarly, the downstream sequence may be a RNA sequence that shares sequence similarity with the RNA sequence downstream of the targeted site of integration. The upstream and downstream sequences in the exogenous RNA template can have 75%, 80%, 85%, 90%, 95%, or 100% sequence identity with the targeted RNA sequence. Preferably, the upstream and downstream sequences in the exogenous RNA template have about 95%, 96%, 97%, 98%, 99%, or 100% sequence identity with the targeted RNA sequence. In some cases, the upstream and downstream sequences in the exogenous RNA template have about 99% or 100% sequence identity with the targeted RNA sequence. An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000 bp. In some methods, the exogenous RNA template may further comprise a marker. Such a marker may make it easy to screen for targeted integrations. Examples of suitable markers include restriction sites, fluorescent proteins, or selectable markers. The exogenous RNA template of the invention can be constructed using recombinant techniques (see, for example, Sambrook et al., 2001 and Ausubel et al., 1996). In a method for modifying a target RNA by integrating an exogenous RNA template, a break (e.g., double or single stranded break in double or single stranded RNA) is introduced into the RNA sequence by the nucleic acid-targeting complex, the break is repaired via homologous recombination with an exogenous RNA template such that the template is integrated into the RNA target. The presence of a double-stranded break facilitates integration of the template. In other embodiments, this invention provides a method of modifying expression of a RNA in a eukaryotic cell. The method comprises increasing or decreasing expression of a target polynucleotide by using a nucleic acid-targeting complex that binds to the DNA or RNA (e.g., mRNA or pre-mRNA). In some methods, a target RNA can be inactivated to affect the modification of the expression in a cell. For example, upon the binding of a RNA-targeting complex to a target sequence in a cell, the target RNA is inactivated such that the sequence is not translated, the coded protein is not produced, or the sequence does not function as the wild-type sequence does. For example, a protein or microRNA coding sequence may be inactivated such that the protein or microRNA or pre-microRNA transcript is not produced. The target RNA of a RNA-targeting complex can be any RNA endogenous or exogenous to the eukaryotic cell. For example, the target RNA can be a RNA residing in the nucleus of the eukaryotic cell. The target RNA can be a sequence (e.g., mRNA or pre-mRNA) coding a gene product (e.g., a protein) or a non-coding sequence (e.g., ncRNA, lncRNA, tRNA, or rRNA). Examples of target RNA include a sequence associated with a signaling biochemical pathway, e.g., a signaling biochemical pathway-associated RNA. Examples of target RNA include a disease associated RNA. A “disease-associated” RNA refers to any RNA which is yielding translation products at an abnormal level or in an abnormal form in cells derived from a disease-affected tissues compared with tissues or cells of a non-disease control. It may be a RNA transcribed from a gene that becomes expressed at an abnormally high level; it may be a RNA transcribed from a gene that becomes expressed at an abnormally low level, where the altered expression correlates with the occurrence and/or progression of the disease. A disease-associated RNA also refers to a RNA transcribed from a gene possessing mutation(s) or genetic variation that is directly responsible or is in linkage disequilibrium with a gene(s) that is responsible for the etiology of a disease. The translated products may be known or unknown, and may be at a normal or abnormal level. The target RNA of a RNA-targeting complex can be any RNA endogenous or exogenous to the eukaryotic cell. For example, the target RNA can be a RNA residing in the nucleus of the eukaryotic cell. The target RNA can be a sequence (e.g., mRNA or pre-mRNA) coding a gene product (e.g., a protein) or a non-coding sequence (e.g., ncRNA, lncRNA, tRNA, or rRNA).

In some embodiments, the systems and methods may comprise allowing a RNA-targeting complex to bind to the target RNA to effect cleavage of said target RNA thereby modifying the target RNA, wherein the RNA-targeting complex comprises a nucleic acid-targeting effector (Cas13) protein complexed with a guide RNA or crRNA hybridized to a target sequence within said target RNA. In one aspect, the invention provides a method of modifying expression of RNA in a eukaryotic cell. In some embodiments, the method comprises allowing a RNA-targeting complex to bind to the RNA such that said binding results in increased or decreased expression of said RNA; wherein the RNA-targeting complex comprises a nucleic acid-targeting effector (Cas13) protein complexed with a guide RNA. Methods of modifying a target RNA can be in a eukaryotic cell, which may be in vivo, ex vivo or in vitro. In some embodiments, the method comprises sampling a cell or population of cells from a human or non-human animal, and modifying the cell or cells. Culturing may occur at any stage ex vivo. The cell or cells may even be re-introduced into the non-human animal or plant. For re-introduced cells it is particularly preferred that the cells are stem cells.

The use of two different aptamers (each associated with a distinct RNA-targeting guide RNAs) allows an activator-adaptor protein fusion and a repressor-adaptor protein fusion to be used, with different RNA-targeting guide RNAs or crRNAs, to activate expression of RNA, whilst repressing another. They, along with their different guide RNAs or crRNAs can be administered together, or substantially together, in a multiplexed approach. A large number of such modified RNA-targeting guide RNAs or crRNAs can be used all at the same time, for example 10 or 20 or 30 and so forth, whilst only one (or at least a minimal number) of effector protein (Cas13) molecules need to be delivered, as a comparatively small number of effector protein molecules can be used with a large number of modified guides. The adaptor protein may be associated (preferably linked or fused to) one or more activators or one or more repressors. For example, the adaptor protein may be associated with a first activator and a second activator. The first and second activators may be the same, but they are preferably different activators. Three or more or even four or more activators (or repressors) may be used, but package size may limit the number being higher than 5 different functional domains. Linkers are preferably used, over a direct fusion to the adaptor protein, where two or more functional domains are associated with the adaptor protein. Suitable linkers might include the GlySer linker.

CRISPR effector (Cas13) protein or mRNA therefor (or more generally a nucleic acid molecule therefor) and guide RNA or crRNA might also be delivered separately e.g., the former 1-12 hours (preferably around 2-6 hours) prior to the administration of guide RNA or crRNA, or together. A second booster dose of guide RNA or crRNA can be administered 1-12 hours (preferably around 2-6 hours) after the initial administration.

The Cas13 effector protein is sometimes referred to herein as a CRISPR Enzyme. It will be appreciated that the effector protein is based on orderived from an enzyme, so the term ‘effector protein’ certainly includes ‘enzyme’ in some embodiments. However, it will also be appreciated that the effector protein may, as required in some embodiments, have DNA or RNA binding, but not necessarily cutting or nicking, activity, including a dead-Cas effector protein function.

Cellular targets include Hemopoietic Stem/Progenitor Cells (CD34+); Human T cells; and Eye (retinal cells)—for example photoreceptor precursor cells.

The systems may comprise templates. Delivery of templates may be via the cotemporaneous or separate from delivery of any or all the CRISPR effector protein (Cas13) or guide or crRNA and via the same delivery mechanism or different.

In certain embodiments, the methods as described herein may comprise providing a Cas13 transgenic cell in which one or more nucleic acids encoding one or more guide RNAs are provided or introduced operably connected in the cell with a regulatory element comprising a promoter of one or more gene of interest. As used herein, the term “Cas13 transgenic cell” refers to a cell, such as a eukaryotic cell, in which a Cas13 gene has been genomically integrated. The nature, type, or origin of the cell are not particularly limiting according to the present invention. Also the way how the Cas13 transgene is introduced in the cell is may vary and can be any method as is known in the art. In certain embodiments, the Cas13 transgenic cell is obtained by introducing the Cas13 transgene in an isolated cell. In certain other embodiments, the Cas13 transgenic cell is obtained by isolating cells from a Cas13 transgenic organism. By means of example, and without limitation, the Cas13 transgenic cell as referred to herein may be derived from a Cas13 transgenic eukaryote, such as a Cas13 knock-in eukaryote. Reference is made to WO 2014/093622 (PCT/US13/74667), incorporated herein by reference. Methods of US Patent Publication Nos. 20120017290 and 20110265198 assigned to Sangamo BioSciences, Inc. directed to targeting the Rosa locus may be modified to utilize the CRISPR Cas system of the present invention. Methods of US Patent Publication No. 20130236946 assigned to Cellectis directed to targeting the Rosa locus may also be modified to utilize the CRISPR Cas system of the present invention. By means of further example reference is made to Platt et. al. (Cell; 159(2):440-455 (2014)), describing a Cas9 knock-in mouse, which is incorporated herein by reference. The Cas13 transgene can further comprise a Lox-Stop-polyA-Lox(LSL) cassette thereby rendering Cas13 expression inducible by Cre recombinase. Alternatively, the Cas13 transgenic cell may be obtained by introducing the Cas13 transgene in an isolated cell. Delivery systems for transgenes are well known in the art. By means of example, the Cas13 transgene may be delivered in for instance eukaryotic cell by means of vector (e.g., AAV, adenovirus, lentivirus) and/or particle and/or particle delivery, as also described herein elsewhere.

It will be understood by the skilled person that the cell, such as the Cas13 transgenic cell, as referred to herein may comprise further genomic alterations besides having an integrated Cas13 gene or the mutations arising from the sequence specific action of Cas13 when complexed with RNA capable of guiding Cas13 to a target locus, such as for instance one or more oncogenic mutations, as for instance and without limitation described in Platt et al. (2014), Chen et al., (2014) or Kumar et al., (2009).

The guide RNA(s), e.g., sgRNA(s) or crRNA(s) encoding sequences and/or Cas13 encoding sequences, can be functionally or operatively linked to regulatory element(s) and hence the regulatory element(s) drive expression. The promoter(s) can be constitutive promoter(s) and/or conditional promoter(s) and/or inducible promoter(s) and/or tissue specific promoter(s). The promoter can be selected from the group consisting of RNA polymerases, pol I, pol II, pol III, T7, U6, H1, retroviral Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, and the EF1α promoter. An advantageous promoter is the promoter is U6.

In some embodiments, a Cas protein (e.g., Cas13 protein) may form a component of an inducible system. The inducible nature of the system would allow for spatiotemporal control of gene editing or gene expression using a form of energy. The form of energy may include but is not limited to electromagnetic radiation, sound energy, chemical energy and thermal energy. Examples of inducible system include tetracycline inducible promoters (Tet-On or Tet-Off), small molecule two-hybrid transcription activations systems (FKBP, ABA, etc.), or light inducible systems (Phytochrome, LOV domains, or cryptochrome). In one embodiment, the CRISPR effector protein may be a part of a Light Inducible Transcriptional Effector (LITE) to direct changes in transcriptional activity in a sequence-specific manner. The components of a light may include a CRISPR effector protein, a light-responsive cytochrome heterodimer (e.g. from Arabidopsis thaliana), and a transcriptional activation/repression domain. Further examples of inducible DNA binding proteins and methods for their use are provided in U.S. 61/736,465 and U.S. 61/721,283, and WO 2014018423 A2 which is hereby incorporated by reference in its entirety.

In one aspect, the invention provides a mutated Cas13 as described herein, having one or more mutations resulting in reduced off-target effects, i.e. improved CRISPR enzymes for use in effecting modifications to target loci but which reduce or eliminate activity towards off-targets, such as when complexed to guide RNAs, as well as improved CRISPR enzymes for increasing the activity of CRISPR enzymes, such as when complexed with guide RNAs. It is to be understood that mutated enzymes as described herein below may be used in any of the methods according to the invention as described herein elsewhere. Any of the methods, products, compositions and uses as described herein elsewhere are equally applicable with the mutated CRISPR enzymes as further detailed below.

Slaymaker et al. recently described a method for the generation of Cas9 orthologs with enhanced specificity (Slaymaker et al. 2015 “Rationally engineered Cas9 nucleases with improved specificity”). This strategy can be used to enhance the specificity of the Cas13 protein. Primary residues for mutagenesis are preferably all positive charges residues within the HEPN domain. Additional residues are positive charged residues that are conserved between different orthologs.

In an aspect, the invention also provides methods and mutations for modulating Cas13 binding activity and/or binding specificity. In certain embodiments Cas13 proteins lacking nuclease activity are used. In certain embodiments, modified guide RNAs are employed that promote binding but not nuclease activity of a Cas13 nuclease. In such embodiments, on-target binding can be increased or decreased. Also, in such embodiments off-target binding can be increased or decreased. Moreover, there can be increased or decreased specificity as to on-target binding vs. off-target binding.

The methods and mutations which can be employed in various combinations to increase or decrease activity and/or specificity of on-target vs. off-target activity, or increase or decrease binding and/or specificity of on-target vs. off-target binding, can be used to compensate or enhance mutations or modifications made to promote other effects. Such mutations or modifications made to promote other effects in include mutations or modification to the Cas13 and or mutation or modification made to a guide RNA. The methods and mutations of the invention are used to modulate Cas13 nuclease activity and/or binding with chemically modified guide RNAs.

In an aspect, the invention provides methods and mutations for modulating binding and/or binding specificity of Cas13 proteins according to the invention as defined herein comprising functional domains such as nucleases, transcriptional activators, transcriptional repressors, and the like. For example, a Cas13 protein can be made nuclease-null, or having altered or reduced nuclease activity by introducing mutations such as for instance Cas13 mutations described herein elsewhere. Nuclease deficient Cas13 proteins are useful for RNA-guided target sequence dependent delivery of functional domains. The invention provides methods and mutations for modulating binding of Cas13 proteins. In one embodiment, the functional domain comprises VP64, providing an RNA-guided transcription factor. In another embodiment, the functional domain comprises Fok I, providing an RNA-guided nuclease activity. Mention is made of U.S. Pat. Pub. 2014/0356959, U.S. Pat. Pub. 2014/0342456, U.S. Pat. Pub. 2015/0031132, and Mali, P. et al., 2013, Science 339(6121):823-6, doi: 10.1126/science.1232033, published online 3 Jan. 2013 and through the teachings herein the invention comprehends methods and materials of these documents applied in conjunction with the teachings herein. In certain embodiments, on-target binding is increased. In certain embodiments, off-target binding is decreased. In certain embodiments, on-target binding is decreased. In certain embodiments, off-target binding is increased. Accordingly, the invention also provides for increasing or decreasing specificity of on-target binding vs. off-target binding of functionalized Cas13 binding proteins.

The use of Cas13 as an RNA-guided binding protein is not limited to nuclease-null Ca13. Cas13 enzymes comprising nuclease activity can also function as RNA-guided binding proteins when used with certain guide RNAs. For example short guide RNAs and guide RNAs comprising nucleotides mismatched to the target can promote RNA directed Cas13 binding to a target sequence with little or no target cleavage. (See, e.g., Dahlman, 2015, Nat Biotechnol. 33(11):1159-1161, doi: 10.1038/nbt.3390, published online 5 Oct. 2015). In an aspect, the invention provides methods and mutations for modulating binding of Cas13 proteins that comprise nuclease activity. In certain embodiments, on-target binding is increased. In certain embodiments, off-target binding is decreased. In certain embodiments, on-target binding is decreased. In certain embodiments, off-target binding is increased. In certain embodiments, there is increased or decreased specificity of on-target binding vs. off-target binding. In certain embodiments, nuclease activity of guide RNA-Cas13 enzyme is also modulated.

RNA-RNA duplex formation is important for cleavage activity and specificity throughout the target region, not only the seed region sequence closest to the PFS. Thus, truncated guide RNAs show reduced cleavage activity and specificity. In an aspect, the invention provides method and mutations for increasing activity and specificity of cleavage using altered guide RNAs.

In certain embodiments, the catalytic activity of the Cas protein (e.g., Cas13) of the invention is altered or modified. It is to be understood that mutated Cas13 has an altered or modified catalytic activity if the catalytic activity is different than the catalytic activity of the corresponding wild type CRISPR-Cas protein (e.g., unmutated CRISPR-Cas protein). Catalytic activity can be determined by means known in the art. By means of example, and without limitation, catalytic activity can be determined in vitro or in vivo by determination of indel percentage (for instance after a given time, or at a given dose). In certain embodiments, catalytic activity is increased. In certain embodiments, catalytic activity is increased by at least 5%, preferably at least 10%, more preferably at least 20%, such as at least 30%, at least 40%, at least 50%, at least 60%, at least 70%, at least 80%, at least 90%, or at least 100%. In certain embodiments, catalytic activity is decreased. In certain embodiments, catalytic activity is decreased by at least 5%, preferably at least 10%, more preferably at least 20%, such as at least 30%, at least 40%, at least 50%, at least 60%, at least 70%, at least 80%, at least 90%, or (substantially) 100%. The one or more mutations herein may inactivate the catalytic activity, which may substantially all catalytic activity, below detectable levels, or no measurable catalytic activity.

One or more characteristics of the engineered CRISPR-Cas protein may be different from a corresponding wiled type CRISPR-Cas protein. Examples of such characteristics include catalytic activity, gRNA binding, specificity of the CRISPR-Cas protein (e.g., specificity of editing a defined target), stability of the CRISPR-Cas protein, off-target binding, target binding, protease activity, nickase activity, PFS recognition. In some examples, a engineered CRISPR-Cas protein may comprise one or more mutations of the corresponding wild type CRISPR-Cas protein. In some embodiments, the catalytic activity of the engineered CRISPR-Cas protein is increased as compared to a corresponding wildtype CRISPR-Cas protein. In some embodiments, the catalytic activity of the engineered CRISPR-Cas protein is decreased as compared to a corresponding wildtype CRISPR-Cas protein. In some embodiments, the gRNA binding of the engineered CRISPR-Cas protein is increased as compared to a corresponding wildtype CRISPR-Cas protein. In some embodiments, the gRNA binding of the engineered CRISPR-Cas protein is decreased as compared to a corresponding wildtype CRISPR-Cas protein. In some embodiments, the specificity of the CRISPR-Cas protein is increased as compared to a corresponding wildtype CRISPR-Cas protein. In some embodiments, the specificity of the CRISPR-Cas protein is decreased as compared to a corresponding wildtype CRISPR-Cas protein. In some embodiments, the stability of the CRISPR-Cas protein is increased as compared to a corresponding wildtype CRISPR-Cas protein. In some embodiments, the stability of the CRISPR-Cas protein is decreased as compared to a corresponding wildtype CRISPR-Cas protein. In some embodiments, the engineered CRISPR-Cas protein further comprises one or more mutations which inactivate catalytic activity. In some embodiments, the off-target binding of the CRISPR-Cas protein is increased as compared to a corresponding wildtype CRISPR-Cas protein. In some embodiments, the off-target binding of the CRISPR-Cas protein is decreased as compared to a corresponding wildtype CRISPR-Cas protein. In some embodiments, the target binding of the CRISPR-Cas protein is increased as compared to a corresponding wildtype CRISPR-Cas protein. In some embodiments, the target binding of the CRISPR-Cas protein is decreased as compared to a corresponding wildtype CRISPR-Cas protein. In some embodiments, the engineered CRISPR-Cas protein has a higher protease activity or polynucleotide-binding capability compared with a corresponding wildtype CRISPR-Cas protein. In some embodiments, the PFS recognition is altered as compared to a corresponding wildtype CRISPR-Cas protein.

In certain embodiments, the gRNA (crRNA) binding of the Cas13 protein of the invention is altered or modified. It is to be understood that mutated Cas13 has an altered or modified gRNA binding if the gRNA binding is different than the gRNA binding of the corresponding wild type Cas13 (i.e. unmutated Cas13).gRNA binding can be determined by means known in the art. By means of example, and without limitation, gRNA binding can be determined by calculating binding strength or affinity (such as based on equilibrium constants, Ka, Kd, etc). In certain embodiments, gRNA binding is increased. In certain embodiments, gRNA binding is increased by at least 5%, preferably at least 10%, more preferably at least 20%, such as at least 30%, at least 40%, at least 50%, at least 60%, at least 70%, at least 80%, at least 90%, or at least 100%. In certain embodiments, gRNA binding is decreased. In certain embodiments, gRNA binding is decreased by at least 5%, preferably at least 10%, more preferably at least 20%, such as at least 30%, at least 40%, at least 50%, at least 60%, at least 70%, at least 80%, at least 90%, or (substantially) 100%.

In certain embodiments, the specificity of the Cas13 protein of the invention is altered or modified. It is to be understood that mutated Cas13 has an altered or modified specificity if the specificity is different than the specificity of the corresponding wild type Cas13 (i.e. unmutated Cas13). Specificity can be determined by means known in the art. By means of example, and without limitation, specificity can be determined by comparison of on-target activity and off-target activity. In certain embodiments, specificity is increased. In certain embodiments, specificity is increased by at least 5%, preferably at least 10%, more preferably at least 20%, such as at least 30%, at least 40%, at least 50%, at least 60%, at least 70%, at least 80%, at least 90%, or at least 100%. In certain embodiments, specificity is decreased. In certain embodiments, specificity is decreased by at least 5%, preferably at least 10%, more preferably at least 20%, such as at least 30%, at least 40%, at least 50%, at least 60%, at least 70%, at least 80%, at least 90%, or (substantially) 100%.

In certain embodiments, the stability of the Cas13 protein of the invention is altered or modified. It is to be understood that mutated Cas13 has an altered or modified stability if the stability is different than the stability of the corresponding wild type Cas13 (i.e. unmutated Cas13). Stability can be determined by means known in the art. By means of example, and without limitation, stability can be determined by determining the half-life of the Cas13 protein. In certain embodiments, stability is increased. In certain embodiments, stability is increased by at least 5%, preferably at least 10%, more preferably at least 20%, such as at least 30%, at least 40%, at least 50%, at least 60%, at least 70%, at least 80%, at least 90%, or at least 100%. In certain embodiments, stability is decreased. In certain embodiments, stability is decreased by at least 5%, preferably at least 10%, more preferably at least 20%, such as at least 30%, at least 40%, at least 50%, at least 60%, at least 70%, at least 80%, at least 90%, or (substantially) 100%.

In certain embodiments, the target binding of the Cas13 protein of the invention is altered or modified. It is to be understood that mutated Cas13 has an altered or modified target binding if the target binding is different than the target binding of the corresponding wild type Cas13 (i.e. unmutated Cas13). target binding can be determined by means known in the art. By means of example, and without limitation, target binding can be determined by calculating binding strength or affinity (such as based on equilibrium constants, Ka, Kd, etc). In certain embodiments, target bindings increased. In certain embodiments, target binding is increased by at least 5%, preferably at least 10%, more preferably at least 20%, such as at least 30%, at least 40%, at least 50%, at least 60%, at least 70%, at least 80%, at least 90%, or at least 100%. In certain embodiments, target binding is decreased. In certain embodiments, target binding is decreased by at least 5%, preferably at least 10%, more preferably at least 20%, such as at least 30%, at least 40%, at least 50%, at least 60%, at least 70%, at least 80%, at least 90%, or (substantially) 100%.

In certain embodiments, the off-target binding of the Cas13 protein of the invention is altered or modified. It is to be understood that mutated Cas13 has an altered or modified off-target binding if the off-target binding is different than the off-target binding of the corresponding wild type Cas13 (i.e. unmutated Cas13). Off-target binding can be determined by means known in the art. By means of example, and without limitation, off-target binding can be determined by calculating binding strength or affinity (such as based on equilibrium constants, Ka, Kd, etc). In certain embodiments, off-target bindings increased. In certain embodiments, off-target binding is increased by at least 5%, preferably at least 10%, more preferably at least 20%, such as at least 30%, at least 40%, at least 50%, at least 60%, at least 70%, at least 80%, at least 90%, or at least 100%. In certain embodiments, off-target binding is decreased. In certain embodiments, off-target binding is decreased by at least 5%, preferably at least 10%, more preferably at least 20%, such as at least 30%, at least 40%, at least 50%, at least 60%, at least 70%, at least 80%, at least 90%, or (substantially) 100%.

In certain embodiments, the PFS recognition or specificity of the Cas13 protein of the invention is altered or modified. It is to be understood that mutated Cas13 has an altered or modified PFS recognition or specificity if the PFS recognition or specificity is different than the PFS recognition or specificity of the corresponding wild type Cas13 (i.e. unmutated Cas13). PFS recognition or specificity can be determined by means known in the art. By means of example, and without limitation, PFS recognition or specificity can be determined by PFS screens. In certain embodiments, at least one different PFS is recognized by the Cas13. In certain embodiments, at least one PFS is recognized by the mutated Cas13 which is not recognized by the corresponding wild type Cas13. In certain embodiments, at least one PFS is recognized by the mutated Cas13 which is not recognized by the corresponding wild type Cas13, in addition to the wild type PFS. In certain embodiments, at least one PFS is recognized by the mutated Cas13 which is not recognized by the corresponding wild type Cas13, and the wild type PFS is not anymore recognized. In certain embodiments, the PFS recognized by the mutated Cas13 is longer than the PFS recognized by the wild type Cas13, such as 1, 2, or 3 nucleotides longer. In certain embodiments, the PFS recognized by the mutated Cas13 is shorter than the PFS recognized by the wild type Cas13, such as 1, 2, or 3 nucleotides shorter.

In some embodiments, the invention provides a non-naturally occurring or engineered composition comprising i) a mutated Cas13 effector protein, and ii) a crRNA, wherein the crRNA comprises a) a guide sequence that is capable of hybridizing to a target RNA sequence, and b) a direct repeat sequence, whereby there is formed a CRISPR complex comprising the Cas13 effector protein complexed with the guide sequence that is hybridized to the target RNA sequence. The complex can be formed in vitro or ex vivo and introduced into a cell or contacted with RNA; or can be formed in vivo.

In some embodiments, such as for Cas13, a non-naturally occurring or engineered composition of the invention may comprise an accessory protein that enhances Type VI Cas protein activity. In such embodiments, the Type VI Cas protein and the Type VI CRISPR-Cas accessory protein may be from the same source or from a different source. In some embodiments, a non-naturally occurring or engineered composition of the invention comprises an accessory protein that represses Cas13 protein activity. In some embodiments, a non-naturally occurring or engineered composition of the invention comprises two or more crRNAs. In some embodiments, a non-naturally occurring or engineered composition of the invention comprises a guide sequence that hybridizes to a target RNA sequence in a prokaryotic cell. In some embodiments, a non-naturally occurring or engineered composition of the invention comprises a guide sequence that hybridizes to a target RNA sequence in a eukaryotic cell. In some embodiments, the Cas13 protein comprises one or more nuclear localization signals (NLSs).

In some embodiments of the non-naturally occurring or engineered composition of the invention, the Cas13 protein and the accessory protein are from the same organism.

In some embodiments of the non-naturally occurring or engineered composition of the invention, the Cas13 protein and the accessory protein are from different organisms.

The invention also provides a Type VI CRISPR-Cas vector system, which comprises one or more vectors comprising: a first regulatory element operably linked to a nucleotide sequence encoding the Cas13 effector protein, and a second regulatory element operably linked to a nucleotide sequence encoding the crRNA.

In certain embodiments, the vector system of the invention further comprises a regulatory element operably linked to a nucleotide sequence of a Type VI CRISPR-Cas accessory protein.

When appropriate, the nucleotide sequence encoding the Type VI CRISPR-Cas effector protein (and/or optionally the nucleotide sequence encoding the Type VI CRISPR-Cas accessory protein) is codon optimized for expression in a eukaryotic cell.

In some embodiments of the vector system of the invention, the nucleotide sequences encoding the Cas13 effector protein (and optionally) the accessory protein are codon optimized for expression in a eukaryotic cell.

In some embodiments, the vector system of the invention comprises in a single vector. In some embodiment of the vector system of the invention, the one or more vectors comprise viral vectors. In some embodiment of the vector system of the invention, the one or more vectors comprise one or more retroviral, lentiviral, adenoviral, adeno-associated or herpes simplex viral vectors.

In some embodiments, the invention provides a delivery system configured to deliver a Cas13 effector protein and one or more nucleic acid components of a non-naturally occurring or engineered composition comprising i) a mutated Cas13 effector protein according to the invention as described herein, and ii) a crRNA, wherein the crRNA comprises a) a guide sequence that hybridizes to a target RNA sequence in a cell, and b) a direct repeat sequence, wherein the Cas13 effector protein forms a complex with the crRNA, wherein the guide sequence directs sequence-specific binding to the target RNA sequence, whereby there is formed a CRISPR complex comprising the Cas13 effector protein complexed with the guide sequence that is hybridized to the target RNA sequence. The complex can be formed in vitro or ex vivo and introduced into a cell or contacted with RNA; or can be formed in vivo.

In some embodiments of the delivery system of the invention, the system comprises one or more vectors or one or more polynucleotide molecules, the one or more vectors or polynucleotide molecules comprising one or more polynucleotide molecules encoding the Cas13 effector protein and one or more nucleic acid components of the non-naturally occurring or engineered composition.

In some embodiments, the delivery system of the invention comprises a delivery vehicle comprising liposome(s), particle(s), exosome(s), microvesicle(s), a gene-gun or one or more viral vector(s). In some embodiment, the non-naturally occurring or engineered composition of the invention is for use in a therapeutic method of treatment or in a research program. In some embodiment, the non-naturally occurring or engineered vector system of the invention is for use in a therapeutic method of treatment or in a research program. In some embodiment, the non-naturally occurring or engineered delivery system of the invention is for use in a therapeutic method of treatment or in a research program.

In some embodiments of the invention provides a method of modifying expression of a target gene of interest, the method comprising contacting a target RNA with one or more non-naturally occurring or engineered compositions comprising i) a mutated Cas13 effector protein according to the invention as described herein, and ii) a crRNA, wherein the crRNA comprises a) a guide sequence that hybridizes to a target RNA sequence in a cell, and b) a direct repeat sequence, wherein the Cas13 effector protein forms a complex with the crRNA, wherein the guide sequence directs sequence-specific binding to the target RNA sequence in a cell, whereby there is formed a CRISPR complex comprising the Cas13 effector protein complexed with the guide sequence that is hybridized to the target RNA sequence, whereby expression of the target locus of interest is modified. The complex can be formed in vitro or ex vivo and introduced into a cell or contacted with RNA; or can be formed in vivo.

In some embodiments, the method of modifying expression of a target gene of interest further comprises contacting the target RNA with an accessory protein that enhances Cas13 effector protein activity.

In some embodiments of the method of modifying expression of a target gene of interest, the accessory protein that enhances Cas13 effector protein activity is a csx28 protein.

In some embodiments, the method of modifying expression of a target gene of interest further comprises contacting the target RNA with an accessory protein that represses Cas13 protein activity.

In some embodiments of the method of modifying expression of a target gene of interest, the accessory protein that represses Cas13 effector protein activity is a csx27 protein.

In some embodiments, the method of modifying expression of a target gene of interest comprises cleaving the target RNA.

In some embodiments, the method of modifying expression of a target gene of interest comprises increasing or decreasing expression of the target RNA.

In some embodiments of the method of modifying expression of a target gene of interest, the target gene is in a prokaryotic cell.

In some embodiments of the method of modifying expression of a target gene of interest, the target gene is in a eukaryotic cell.

In some embodiments of the invention provides a cell comprising a modified target of interest, wherein the target of interest has been modified according to any of the method disclosed herein.

In some embodiments of the invention, the cell is a prokaryotic cell.

In some embodiments of the invention, the cell is a eukaryotic cell.

In some embodiments, modification of the target of interest in a cell results in: a cell comprising altered expression of at least one gene product; a cell comprising altered expression of at least one gene product, wherein the expression of the at least one gene product is increased; or a cell comprising altered expression of at least one gene product, wherein the expression of the at least one gene product is decreased.

In some embodiments, the cell is a mammalian cell or a human cell.

In some embodiments of the invention provides a cell line of or comprising a cell disclosed herein or a cell modified by any of the methods disclosed herein, or progeny thereof.

In some embodiments of the invention provides a multicellular organism comprising one or more cells disclosed herein or one or more cells modified according to any of the methods disclosed herein.

In some embodiments of the invention provides a plant or animal model comprising one or more cells disclosed herein or one or more cells modified according to any of the methods disclosed herein.

In some embodiments of the invention provides a gene product from a cell or the cell line or the organism or the plant or animal model disclosed herein.

In some embodiment, the amount of gene product expressed is greater than or less than the amount of gene product from a cell that does not have altered expression.

In certain embodiments, the Cas13 protein originates from a species of the genus Alistipes, Anaerosalibacter, Bacteroides, Bacteroidetes, Bergeyella, Blautia, Butyrivibrio, Capnocytophaga, Carnobacterium, Chloroflexus, Chryseobacterium, Clostridium, Demequina, Eubacteriaceae, Eubacterium, Flavobacterium, Fusobacterium, Herbinix, Insolitispirillum, Lachnospiraceae, Leptotrichia, Listeria, Myroides, Paludibacter, Phaeodactylibacter, Porphyromonadaceae, Porphyromonas, Prevotella, Pseudobutyrivibrio, Psychroflexus, Reichenbachiella, Rhodobacter, Riemerella, Sinomicrobium, Thalassospira, Ruminococcus. As used herein, when a Cas13 protein originates form a species, it may be the wild type Cas13 protein in the species, or a homolog of the wild type Cas13 protein in the species. The Cas13 protein that is a homolog of the wild type Cas13 protein in the species may comprise one or more variations (e.g., mutations, truncations, etc.) of the wild type Cas13 protein.

In certain embodiments, the Cas13 protein originates from Leptotrichia shahii, Listeria seeligeri, Lachnospiraceae bacterium (such as Lb MA2020, Lb NK4A179, Lb NK4A144), Clostridium aminophilum (such as Ca DSM 10710), Carnobacterium gallinarum (such as Cg DSM 4847), Paludibacter propionicigenes (such as Pp WB4), Listeria weihenstephanensis (such as Lw FSL R9-0317), Listeriaceae bacterium (such as Lb FSL M6-0635), Leptotrichia wadei (such as Lw F0279), Rhodobacter capsulatus (such as Rc SB 1003, Rc R121, Rc DE442), Leptotrichia buccalis (such as Lb C-1013-b), Herbinix hemicellulosilytica, Eubacteriaceae bacterium (such as Eb CTHKCI004), Blautia. sp Marseille-P2398, Leptotrichia sp. oral taxon 879 str. F0557, Chloroflexus aggregans, Demequina aurantiaca, Thalassospira sp. TSL5-1, Pseudobutyrivibrio sp. OR37, Butyrivibrio sp. YAB3001, Leptotrichia sp. Marseille-P3007, Bacteroides ihuae, Porphyromonadaceae bacterium (such as Pb KH3CP3RA), Listeria riparia, Insolitispirillum peregrinum, Alistipes sp. ZOR0009, Bacteroides pyogenes (such as Bp F0041), Bacteroidetes bacterium (such as Bb GWA2_31_9), Bergeyella zoohelcum (such as Bz ATCC 43767), Capnocytophaga canimorsus, Capnocytophaga cynodegmi, Chryseobacterium carnipullorum, Chryseobacterium jejuense, Chryseobacterium ureilyticum, Flavobacterium branchiophilum, Flavobacterium columnare, Flavobacterium sp. 316, Myroides odoratimimus (such as Mo CCUG 10230, Mo CCUG 12901, Mo CCUG 3837), Paludibacter propionicigenes, Phaeodactylibacter xiamenensis, Porphyromonas gingivalis (such as Pg F0185, Pg F0568, Pg JCVI SC0001, Pg W4087, Porphyromonas gulae, Porphyromonas sp. COT-052 OH4946, Prevotella aurantiaca, Prevotella buccae (such as Pb ATCC 33574), Prevotella falsenii, Prevotella intermedia (such as Pi 17, Pi ZT), Prevotella pallens (such as Pp ATCC 700821), Prevotella pleuritidis, Prevotella saccharolytica (such as Ps F0055), Prevotella sp. MA2016, Prevotella sp. MSX73, Prevotella sp. P4-76, Prevotella sp. P5-119, Prevotella sp. P5-125, Prevotella sp. P5-60, Psychroflexus torquis, Reichenbachiella agariperforans, Riemerella anatipestifer, Sinomicrobium oceani, Fusobacterium necrophorum (such as Fn subsp. funduliforme ATCC 51357, Fn DJ-2, Fn BFTR-1, Fn subsp. Funduliforme), Fusobacterium perfoetens (such as Fp ATCC 29250), Fusobacterium ulcerans (such as Fu ATCC 49185), Anaerosalibacter sp. ND1, Eubacterium siraeum, Ruminococcus flavefaciens (such as Rfx XPD3002), or Ruminococcus albus.

In certain embodiments, the Cas13 is Cas13a and originates from a species of the genus Bacteroides, Blautia, Butyrivibrio, Carnobacterium, Chloroflexus, Clostridium, Demequina, Eubacterium, Herbinix, Insolitispirillum, Lachnospiraceae, Leptotrichia, Listeria, Paludibacter, Porphyromonadaceae, Pseudobutyrivibrio, Rhodobacter, or Thalassospira.

In certain embodiments, the Cas13 is Cas13a and originates from Leptotrichia shahii, Listeria seeligeri, Lachnospiraceae bacterium (such as Lb MA2020, Lb NK4A179, Lb NK4A144), Clostridium aminophilum (such as Ca DSM 10710), Carnobacterium gallinarum (such as Cg DSM 4847), Paludibacter propionicigenes (such as Pp WB4), Listeria weihenstephanensis (such as Lw FSL R9-0317), Listeriaceae bacterium (such as Lb FSL M6-0635), Leptotrichia wadei (such as Lw F0279), Rhodobacter capsulatus (such as Rc SB 1003, Rc R121, Rc DE442), Leptotrichia buccalis (such as Lb C-1013-b), Herbinix hemicellulosilytica, Eubacteriaceae bacterium (such as Eb CHKCI004), Blautia. sp Marseille-P2398, Leptotrichia sp. oral taxon 879 str. F0557, Chloroflexus aggregans, Demequina aurantiaca, Thalassospira sp. TSL5-1, Pseudobutyrivibrio sp. OR37, Butyrivibrio sp. YAB3001, Leptotrichia sp. Marseille-P3007, Bacteroides ihuae, Porphyromonadaceae bacterium (such as Pb KH3CP3RA), Listeria riparia, or Insolitispirillum peregrinum.

In certain embodiments, the Cas13 is Cas13b and originates from a species of the genus Alistipes, Bacteroides, Bacteroidetes, Bergeyella, Capnocytophaga, Chryseobacterium, Flavobacterium, Myroides, Paludibacter, Phaeodactylibacter, Porphyromonas, Prevotella, Psychroflexus, Reichenbachiella, Riemerella, or Sinomicrobium.

In certain embodiments, the Cas13 is Cas13b and originates from Alistipes sp. ZOR0009, Bacteroides pyogenes (such as Bp F0041), Bacteroidetes bacterium (such as Bb GWA2_31_9), Bergeyella zoohelcum (such as Bz ATCC 43767), Capnocytophaga canimorsus, Capnocytophaga cynodegmi, Chryseobacterium carnipullorum, Chryseobacterium jejuense, Chryseobacterium ureilyticum, Flavobacterium branchiophilum, Flavobacterium columnare, Flavobacterium sp. 316, Myroides odoratimimus (such as Mo CCUG 10230, Mo CCUG 12901, Mo CCUG 3837), Paludibacter propionicigenes, Phaeodactylibacter xiamenensis, Porphyromonas gingivalis (such as Pg F0185, Pg F0568, Pg JCVI SC001, Pg W4087, Porphyromonas gulae, Porphyromonas sp. COT-052 OH4946, Prevotella aurantiaca, Prevotella buccae (such as Pb ATCC 33574), Prevotella falsenii, Prevotella intermedia (such as Pi 17, Pi ZT), Prevotella pallens (such as Pp ATCC 700821), Prevotella pleuritidis, Prevotella saccharolytica (such as Ps F0055), Prevotella sp. MA2016, Prevotella sp. MSX73, Prevotella sp. P4-76, Prevotella sp. P5-119, Prevotella sp. P5-125, Prevotella sp. P5-60, Psychroflexus torquis, Reichenbachiella agariperforans, Riemerella anatipestifer, or Sinomicrobium oceani. In some examples, the Cas13 is Riemerella anatipestifer Cas13b. In some examples, the Cas13 is a dead Riemerella anatipestifer Cas13. In some examples, the Cas13 is Prevotella sp. P5-125. In some examples, the Cas13 is a dead Prevotella sp. P5-125.

In certain embodiments, the Cas13 is Cas13c and originates from a species of the genus Fusobacterium or Anaerosalibacter.

In certain embodiments, the Cas13 is Cas13c and originates from Fusobacterium necrophorum (such as Fn subsp. funduliforme ATCC 51357, Fn DJ-2, Fn BFTR-1, Fn subsp. Funduliforme), Fusobacterium perfoetens (such as Fp ATCC 29250), Fusobacterium ulcerans (such as Fu ATCC 49185), or Anaerosalibacter sp. ND1.

In certain embodiments, the Cas13 is Cas13d and originates from a species of the genus Eubacterium or Ruminococcus.

In certain embodiments, the Cas13 is Cas13d and originates from Eubacterium siraeum, Ruminococcus flavefaciens (such as Rfx XPD3002), or Ruminococcus albus.

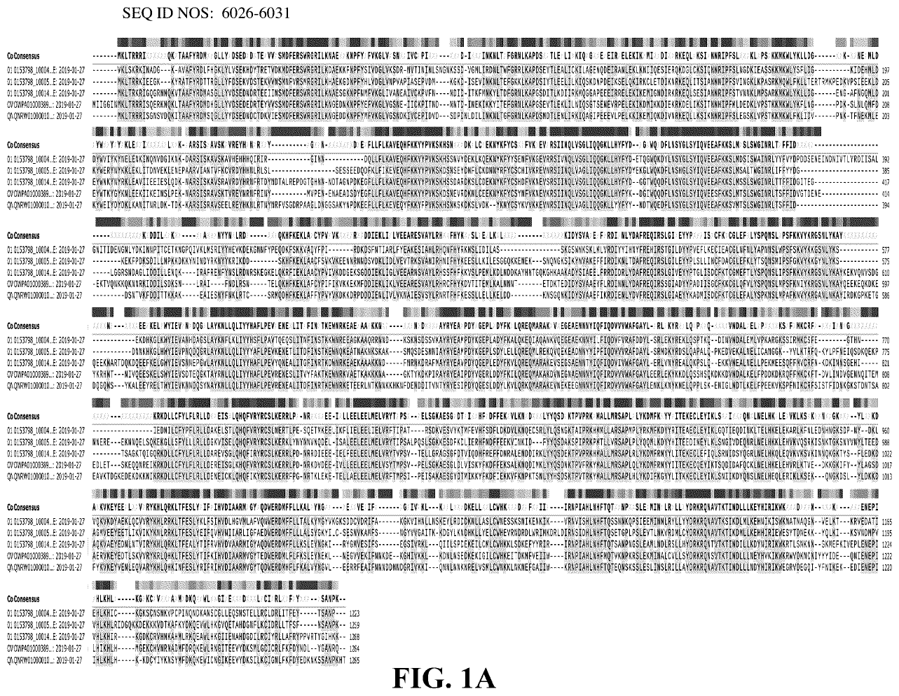

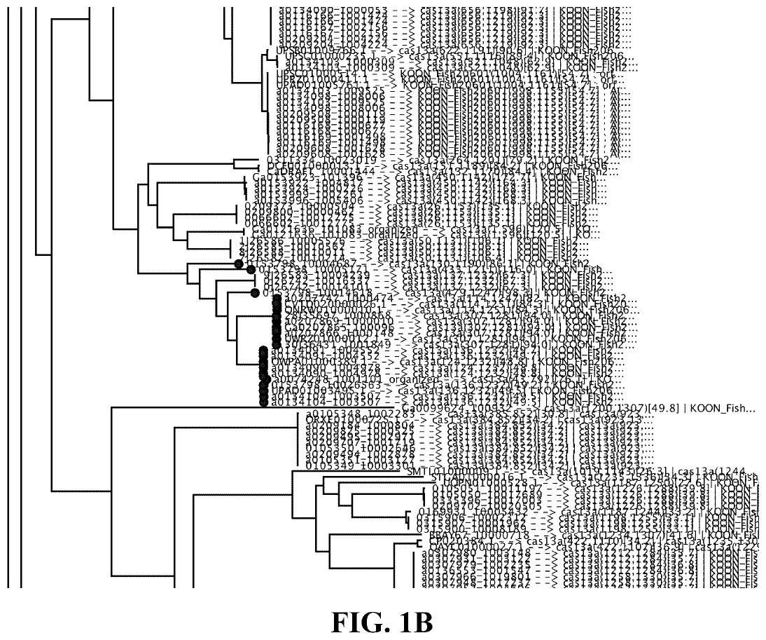

In certain example embodiments, the ortholog selected may be more thermostable at higher temperatures. For example, the ortholog may be thermostable at or above 320° C., 33C, 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C. In certain example embodiments, the ortholog is thermostable at or above 55° C. In certain example embodiments the ortholog is a Cas13a, Cas13b, Cas13c, or Cas13d. In certain example embodiments the ortholog is a Cas13 ortholog. In certain example embodiments, the Cas13a ortholog is derived from Herbinix hemicellulosilytica. In certain example embodiments, the Cas13a ortholog is derived from Herbinix hemicellulosilytica DSM 29228. In certain example embodiments, the Cas 13 ortholog is defined by SEQ ID NO: 1, or by SEQ ID NO: 75 of International Publication No. WO 2017/219027. In certain example embodiments, the Cas 13 ortholog is defined by a sequence from FIG. 1A (loci QNRW01000010.1, OWPA01000389.1, 0153798_10014618, 0153978_10005171, and 0153798_10004687). In certain example embodiments, the Cas 13a ortholog is encoded by the nucleic acid sequence 0123519_10037894 or 0J26742_10014101. In certain other example embodiments, the Cas13 ortholog has at least 80% sequence identity to SEQ ID NO: 1 or to SEQ ID NO: 75 of International Publication No. WO 2017/219027. In certain other example embodiments, the Cas13 ortholog has at least 80% sequence identity to sequence from FIG. 1A (loci QNRW01000010.1, OWPA01000389.1, 0153798_10014618, 0153978_10005171, and 0153798_10004687). In certain other example embodiments, the Cas13 ortholog has at least 80% sequence identity to a polypeptide encoded by the nucleic acid sequence 0123519_10037894 or 0J26742_10014101. In certain example embodiments, the Cas13 ortholog has at least one HEPN domain and at least 80% identity to SEQ ID NO: 1 or to SEQ ID NO: 75 of International Publication No. WO 2017/219027. In certain example embodiments, the Cas13 ortholog has at least one HEPN domain and at least 80% identity to sequence from FIG. 1A (loci QNRW01000010.1, OWPA01000389.1, 0153798_10014618, 0153978_10005171, and 0153798_10004687). In certain example embodiments, the Cas13 ortholog has at least one HEPN domain and at least 80% identity to a polypeptide encoded by the nucleic acid sequence of any one of SEQ ID NOs. 1-4092, 4102-5203, and 5260-5265. In another example embodiment, the Cas13 ortholog has at least two HEPN domains and at least 80% identity to SEQ ID NO: 1 or to SEQ ID NO: 75 of International Publication No. WO 2017/219027. In another example embodiment, the Cas13 ortholog has at least two HEPN domains and at least 80% identity to sequence from FIG. 1A (loci QNRW01000010.1, OWPA01000389.1, 0153798_10014618, 0153978_10005171, and 0153798_10004687). The Cas13a thermostable proteins of FIG. 1A were identified from stable anaerobic thermophilic methanogenic microbiomes fermenting switchgrass, supporting their thermostability. See, Liang et al., Biotechnol Biofuels 2018; 11: 243 doi: 10.1186/s13068-018-1238-1. Similarly, the 0J26742_10014101 clusters with the verified thermophilic sourced Cas13a sequences detailed in FIG. 1A. The nucleic acid identified at loci 123519_10037894 was identified from a study focusing on 70° C. organism. In certain example embodiments, the Cas13 ortholog has at least two HEPN domains and at least 80% identity to a polypeptide encoded by the nucleic acid sequence 0123519_10037894 or 0J26742_10014101. Accordingly, a person of ordinary skill in the art may use characteristics of the above identified orthologs to select other suitable thermostable orthologs from those disclosed herein.

In some embodiments, the invention provides an isolated nucleic acid encoding the Cas13 effector protein. In some embodiments of the invention the isolated nucleic acid comprises DNA sequence and further comprises a sequence encoding a crRNA. The invention provides an isolated eukaryotic cell comprising the nucleic acid encoding the Cas13 effector protein. Thus, herein, “Cas13 effector protein” or “effector protein” or “Cas” or “Cas protein” or “RNA targeting effector protein” or “RNA targeting protein” or like expressions is to be understood as including Cas13a, Cas13b, Cas13c, or Cas13d; expressions such as “RNA targeting CRISPR system” are to be understood as including Cas13a, Cas13b, Cas13c, or Cas13d CRISPR systems; and references to guide RNA or sgRNA are to be read in conjunction with the herein-discussion of the Cas13 system crRNA, e.g., that which is sgRNA in other systems may be considered as or akin to crRNA in the instant invention.

In some embodiments, the invention provides a method of identifying the requirements of a suitable guide sequence for the Cas13 effector protein of the invention, said method comprising: (a) selecting a set of essential genes within an organism, (b) designing a library of targeting guide sequences capable of hybridizing to regions the coding regions of these genes as well as 5′ and 3′ UTRs of these genes, (c) generating randomized guide sequences that do not hybridize to any region within the genome of said organism as control guides, (d) preparing a plasmid comprising the RNA-targeting protein and a first resistance gene and a guide plasmid library comprising said library of targeting guides and said control guides and a second resistance gene, (e) co-introducing said plasmids into a host cell, (f) introducing said host cells on a selective medium for said first and second resistance genes, (g) sequencing essential genes of growing host cells, (h) determining significance of depletion of cells transformed with targeting guides by comparing depletion of cells with control guides; and, (i) determining based on the depleted guide sequences the requirements of a suitable guide sequence.

In one aspect, determining the PFS sequence for suitable guide sequence of the RNA-targeting protein is by comparison of sequences targeted by guides in depleted cells. In one aspect of such method, the method further comprises comparing the guide abundance for the different conditions in different replicate experiments. In one aspect of such method, the control guides are selected in that they are determined to show limited deviation in guide depletion in replicate experiments. In one aspect of such method, the significance of depletion is determined as (a) a depletion which is more than the most depleted control guide; or (b) a depletion which is more than the average depletion plus two times the standard deviation for the control guides. In one aspect of such method, the host cell is a bacterial host cell. In one aspect of such method, the step of co-introducing the plasmids is by electroporation and the host cell is an electro-competent host cell.

In some embodiments, the invention provides a method of modifying sequences associated with or at a target locus of interest, the method comprising delivering to said locus a non-naturally occurring or engineered composition comprising a Cas13 effector protein and one or more nucleic acid components, wherein the effector protein forms a complex with the one or more nucleic acid components and upon binding of the said complex to the locus of interest the effector protein induces the modification of the sequences associated with or at the target locus of interest. In a preferred embodiment, the modification is the introduction of a strand break. In a preferred embodiment, the sequences associated with or at the target locus of interest comprises RNA or consists of RNA.

In some embodiments, the invention provides a method of modifying sequences associated with or at a target locus of interest, the method comprising delivering to said locus a non-naturally occurring or engineered composition comprising a Cas13 effector protein, optionally a small accessory protein, and one or more nucleic acid components, wherein the effector protein forms a complex with the one or more nucleic acid components and upon binding of the said complex to the locus of interest the effector protein induces the modification of the sequences associated with or at the target locus of interest. In a preferred embodiment, the modification is the introduction of a strand break. In a preferred embodiment, the sequences associated with or at the target locus of interest comprises RNA or consists of RNA.

In some embodiments, the invention provides a method of modifying sequences associated with or at a target locus of interest, the method comprising delivering to said sequences associated with or at the locus a non-naturally occurring or engineered composition comprising a Cas13 loci effector protein and one or more nucleic acid components, wherein the Cas13 effector protein forms a complex with the one or more nucleic acid components and upon binding of the said complex to the locus of interest the effector protein induces the modification of sequences associated with or at the target locus of interest. In a preferred embodiment, the modification is the introduction of a strand break. In a preferred embodiment the Cas13 effector protein forms a complex with one nucleic acid component; advantageously an engineered or non-naturally occurring nucleic acid component. The induction of modification of sequences associated with or at the target locus of interest can be Cas13 effector protein-nucleic acid guided. In a preferred embodiment the one nucleic acid component is a CRISPR RNA (crRNA). In a preferred embodiment the one nucleic acid component is a mature crRNA or guide RNA, wherein the mature crRNA or guide RNA comprises a spacer sequence (or guide sequence) and a direct repeat (DR) sequence orderivatives thereof. In a preferred embodiment the spacer sequence or the derivative thereof comprises a seed sequence, wherein the seed sequence is critical for recognition and/or hybridization to the sequence at the target locus. In a preferred embodiment of the invention the crRNA is a short crRNA that may be associated with a short DR sequence. In another embodiment of the invention the crRNA is a long crRNA that may be associated with a long DR sequence (or dual DR). Aspects of the invention relate to Cas13 effector protein complexes having one or more non-naturally occurring or engineered or modified or optimized nucleic acid components. In a preferred embodiment the nucleic acid component comprises RNA. In a preferred embodiment the nucleic acid component of the complex may comprise a guide sequence linked to a direct repeat sequence, wherein the direct repeat sequence comprises one or more stem loops or optimized secondary structures. In preferred embodiments of the invention, the direct repeat may be a short DR or a long DR (dual DR). In a preferred embodiment the direct repeat may be modified to comprise one or more protein-binding RNA aptamers. In a preferred embodiment, one or more aptamers may be included such as part of optimized secondary structure. Such aptamers may be capable of binding a bacteriophage coat protein. The bacteriophage coat protein may be selected from the group comprising Qβ, F2, GA, fr, JP501, MS2, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s and PRR1. In a preferred embodiment the bacteriophage coat protein is MS2. The invention also provides for the nucleic acid component of the complex being 30 or more, 40 or more or 50 or more nucleotides in length.

In some embodiments, the invention provides methods of genome editing or modifying sequences associated with or at a target locus of interest wherein the method comprises introducing a Cas13 complex into any desired cell type, prokaryotic or eukaryotic cell, whereby the Cas13 effector protein complex effectively functions to interfere with RNA in the eukaryotic or prokaryotic cell. In preferred embodiments, the cell is a eukaryotic cell and the RNA is transcribed from a mammalian genome or is present in a mammalian cell. In preferred methods of RNA editing or genome editing in human cells, the Cas13 effector proteins may include but are not limited to the specific species of Cas13 effector proteins disclosed herein.

In some embodiments, the invention also provides a method of modifying a target locus of interest, the method comprising delivering to said locus a non-naturally occurring or engineered composition comprising a Cas13 effector protein and one or more nucleic acid components, wherein the Cas13 effector protein forms a complex with the one or more nucleic acid components and upon binding of the said complex to the locus of interest the effector protein induces the modification of the target locus of interest. In a preferred embodiment, the modification is the introduction of a strand break.

In such methods the target locus of interest may be comprised within a RNA molecule. In such methods the target locus of interest may be comprised in a RNA molecule in vitro.

In such methods the target locus of interest may be comprised in a RNA molecule within a cell. The cell may be a prokaryotic cell or a eukaryotic cell. The cell may be a mammalian cell. The modification introduced to the cell by the present invention may be such that the cell and progeny of the cell are altered for improved production of biologic products such as an antibody, starch, alcohol or other desired cellular output. The modification introduced to the cell by the present invention may be such that the cell and progeny of the cell include an alteration that changes the biologic product produced.

The mammalian cell many be a non-human mammal, e.g., primate, bovine, ovine, porcine, canine, rodent, Leporidae such as monkey, cow, sheep, pig, dog, rabbit, rat or mouse cell. The cell may be a non-mammalian eukaryotic cell such as poultry bird (e.g., chicken), vertebrate fish (e.g., salmon) or shellfish (e.g., oyster, claim, lobster, shrimp) cell. The cell may also be a plant cell. The plant cell may be of a monocot or dicot or of a crop or grain plant such as cassava, corn, sorghum, soybean, wheat, oat or rice. The plant cell may also be of an algae, tree or production plant, fruit or vegetable (e.g., trees such as citrus trees, e.g., orange, grapefruit or lemon trees; peach or nectarine trees; apple or pear trees; nut trees such as almond or walnut or pistachio trees; nightshade plants; plants of the genus Brassica; plants of the genus Lectica; plants of the genus Spinalis; plants of the genus Capsicum; cotton, tobacco, asparagus, carrot, cabbage, broccoli, cauliflower, tomato, eggplant, pepper, lettuce, spinach, strawberry, blueberry, raspberry, blackberry, grape, coffee, cocoa).

In some embodiments, the invention provides a method of modifying a target locus of interest, the method comprising delivering to said locus a non-naturally occurring or engineered composition comprising a Cas13 effector protein and one or more nucleic acid components, wherein the effector protein forms a complex with the one or more nucleic acid components and upon binding of the said complex to the locus of interest the effector protein induces the modification of the target locus of interest. In a preferred embodiment, the modification is the introduction of a strand break.

In such methods the target locus of interest may be comprised within an RNA molecule. In a preferred embodiment, the target locus of interest comprises or consists of RNA.

In some embodiments, the invention also provides a method of modifying a target locus of interest, the method comprising delivering to said locus a non-naturally occurring or engineered composition comprising a Cas13 effector protein and one or more nucleic acid components, wherein the Cas13 effector protein forms a complex with the one or more nucleic acid components and upon binding of the said complex to the locus of interest the effector protein induces the modification of the target locus of interest. In a preferred embodiment, the modification is the introduction of a strand break.

In such methods the target locus of interest may be comprised in a RNA molecule in vitro. In such methods the target locus of interest may be comprised in a RNA molecule within a cell. The cell may be a prokaryotic cell or a eukaryotic cell. The cell may be a mammalian cell. The cell may be a rodent cell. The cell may be a mouse cell.

In any of the described methods the target locus of interest may be a genomic or epigenomic locus of interest. In any of the described methods the complex may be delivered with multiple guides for multiplexed use. In any of the described methods more than one protein(s) may be used.

In further aspects of the invention the nucleic acid components may comprise a CRISPR RNA (crRNA) sequence. As the effector protein is a Cas13 effector protein, the nucleic acid components may comprise a CRISPR RNA (crRNA) sequence and generally may not comprise any trans-activating crRNA (tracr RNA) sequence.

In any of the described methods the effector protein and nucleic acid components may be provided via one or more polynucleotide molecules encoding the protein and/or nucleic acid component(s), and wherein the one or more polynucleotide molecules are operably configured to express the protein and/or the nucleic acid component(s). The one or more polynucleotide molecules may comprise one or more regulatory elements operably configured to express the protein and/or the nucleic acid component(s). The one or more polynucleotide molecules may be comprised within one or more vectors. In any of the described methods the target locus of interest may be a genomic, epigenomic, or transcriptomic locus of interest. In any of the described methods the complex may be delivered with multiple guides for multiplexed use. In any of the described methods more than one protein(s) may be used.

In any of the described methods the strand break may be a single strand break or a double strand break. In preferred embodiments the double strand break may refer to the breakage of two sections of RNA, such as the two sections of RNA formed when a single strand RNA molecule has folded onto itself or putative double helices that are formed with an RNA molecule which contains self-complementary sequences allows parts of the RNA to fold and pair with itself.

Regulatory elements may comprise inducible promotors. Polynucleotides and/or vector systems may comprise inducible systems.

In any of the described methods the one or more polynucleotide molecules may be comprised in a delivery system, or the one or more vectors may be comprised in a delivery system.

In any of the described methods the non-naturally occurring or engineered composition may be delivered via liposomes, particles including nanoparticles, exosomes, microvesicles, a gene-gun or one or more viral vectors.

In some embodiments, the invention also provides a non-naturally occurring or engineered composition which is a composition having the characteristics as discussed herein or defined in any of the herein described methods.

In certain embodiments, the invention thus provides a non-naturally occurring or engineered composition, such as particularly a composition capable of or configured to modify a target locus of interest, said composition comprising a Cas13 effector protein and one or more nucleic acid components, wherein the effector protein forms a complex with the one or more nucleic acid components and upon binding of the said complex to the locus of interest the effector protein induces the modification of the target locus of interest. In certain embodiments, the effector protein may be a Cas13a, Cas13b, Cas13c, or Cas13d effector protein, a Cas13b effector protein.

In certain embodiments, the invention also provides in a further aspect a non-naturally occurring or engineered composition, such as particularly a composition capable of or configured to modify a target locus of interest, said composition comprising: (a) a guide RNA molecule (or a combination of guide RNA molecules, e.g., a first guide RNA molecule and a second guide RNA molecule) or a nucleic acid encoding the guide RNA molecule (or one or more nucleic acids encoding the combination of guide RNA molecules); (b) a Cas13 protein. In certain embodiments, the effector protein may be a Cas13b protein.

In some embodiments, the invention also provides in a further aspect a non-naturally occurring or engineered composition comprising: (I.) one or more CRISPR-Cas system polynucleotide sequences comprising (a) a guide sequence capable of hybridizing to a target sequence in a polynucleotide locus, (b) a tracr mate (i.e. direct repeat) sequence, and (II.) a second polynucleotide sequence encoding a Cas13 effector protein, wherein when transcribed, the guide sequence directs sequence-specific binding of a CRISPR complex to the target sequence, and wherein the CRISPR complex comprises the Cas13 effector protein complexed with the guide sequence that is hybridized to the target sequence. In certain embodiments, the effector protein may be a Cas13 protein.

In certain embodiments, a tracrRNA may not be required. Hence, the invention also provides in certain embodiments a non-naturally occurring or engineered composition comprising: (I.) one or more CRISPR-Cas system polynucleotide sequences comprising (a) a guide sequence capable of hybridizing to a target sequence in a polynucleotide locus, and (b) a direct repeat sequence, and (II.) a second polynucleotide sequence encoding a Cas13 effector protein, wherein when transcribed, the guide sequence directs sequence-specific binding of a CRISPR complex to the target sequence, and wherein the CRISPR complex comprises the Cas13 effector protein complexed with (1) the guide sequence that is hybridized to the target sequence, and (2) the direct repeat sequence. Preferably, the effector protein may be a Cas13 effector protein. Without limitation, the Applicants hypothesize that in such instances, the direct repeat sequence may comprise secondary structure that is sufficient for crRNA loading onto the effector protein. By means of example and not limitation, such secondary structure may comprise, consist essentially of or consist of a stem loop (such as one or more stem loops) within the direct repeat.

In some embodiments, the invention also provides a vector system comprising one or more vectors, the one or more vectors comprising one or more polynucleotide molecules encoding components of a non-naturally occurring or engineered composition which is a composition having the characteristics as defined in any of the herein described methods.

In some embodiments, the invention also provides a delivery system comprising one or more vectors or one or more polynucleotide molecules, the one or more vectors or polynucleotide molecules comprising one or more polynucleotide molecules encoding components of a non-naturally occurring or engineered composition which is a composition having the characteristics discussed herein or as defined in any of the herein described methods.

In some embodiments, the invention also provides a non-naturally occurring or engineered composition, or one or more polynucleotides encoding components of said composition, or vector or delivery systems comprising one or more polynucleotides encoding components of said composition for use in a therapeutic method of treatment. The therapeutic method of treatment may comprise gene or genome editing, or gene therapy.

In some embodiments, the invention also provides for methods and compositions wherein one or more amino acid residues of the effector protein may be modified e.g., an engineered or non-naturally-occurring Cas13 effector protein of or comprising or consisting or consisting essentially a protein from SEQ ID NOs. 1-4092, 4102-5203, and 5260-5265. In an embodiment, the modification may comprise mutation of one or more amino acid residues of the effector protein. The one or more mutations may be in one or more catalytically active domains of the effector protein. The effector protein may have reduced or abolished nuclease activity compared with an effector protein lacking said one or more mutations. The effector protein may not direct cleavage of one RNA strand at the target locus of interest. In a preferred embodiment, the one or more mutations may comprise two mutations. In a preferred embodiment the one or more amino acid residues are modified in the Cas13 effector protein, e.g., an engineered or non-naturally-occurring Cas13 effector protein. In certain embodiments of the invention the effector protein comprises one or more HEPN domains. In a preferred embodiment, the effector protein comprises two HEPN domains. In another preferred embodiment, the effector protein comprises one HEPN domain at the C-terminus and another HEPN domain at the N-terminus of the protein. In certain embodiments, the one or more mutations or the two or more mutations may be in a catalytically active domain of the effector protein comprising a HEPN domain, or a catalytically active domain which is homologous to a HEPN domain. In certain embodiments, the effector protein comprises one or more of the following mutations: R116A, H121A, R1177A, H1182A (wherein amino acid positions correspond to amino acid positions of Group 29 protein originating from Bergeyella zoohelcum ATCC 43767). The skilled person will understand that corresponding amino acid positions in different Cas13 proteins may be mutated to the same effect. In certain embodiments, one or more mutations abolish catalytic activity of the protein completely or partially (e.g. altered cleavage rate, altered specificity, etc.) In certain embodiments, the effector protein as described herein is a “dead” effector protein, such as a dead Cas13 effector protein (dCas13). In certain embodiments, the effector protein has one or more mutations in HEPN domain 1. In certain embodiments, the effector protein has one or more mutations in HEPN domain 2. In certain embodiments, the effector protein has one or more mutations in HEPN domain 1 and HEPN domain 2.

In some embodiments, in certain embodiments, the Cas13 effector proteins herein may be associated with a locus comprising short CRISPR repeats between 30 and 40 bp long, more typically between 34 and 38 bp long, even more typically between 36 and 37 bp long, e.g., 30, 31, 32, 33, 34, 35, 36, 37, 38, 39, or 40 bp long. In certain embodiments the CRISPR repeats are long or dual repeats between 80 and 350 bp long such as between 80 and 200 bp long, even more typically between 86 and 88 bp long, e.g., 80, 81, 82, 83, 84, 85, 86, 87, 88, 89, or 90 bp long

In certain embodiments, a protospacer flanking site (PFS) or protospacer adjacent motif (PAM) or PAM-like motif directs binding of the effector protein (e.g. a Cas13 effector protein) complex as disclosed herein to the target locus of interest. In some embodiments, the PFS may be a 5′ PFS (i.e., located upstream of the 5′ end of the protospacer). In other embodiments, the PFS may be a 3′ PFS (i.e., located downstream of the 5′ end of the protospacer). In other embodiments, both a 5′ PFS and a 3′ PFS are required. In certain embodiments of the invention, a PFS or PFS-like motif may not be required for directing binding of the effector protein (e.g. a Cas13 effector protein). In certain embodiments, a 5′ PFS is D (e.g., A, G, or U). In certain embodiments, a 5′ v is D for Cas13 effectors. In certain embodiments of the invention, cleavage at repeat sequences may generate crRNAs (e.g. short or long crRNAs) containing a full spacer sequence flanked by a short nucleotide (e.g. 5, 6, 7, 8, 9, or 10 nt or longer if it is a dual repeat) repeat sequence at the 5′ end (this may be referred to as a crRNA “tag”) and the rest of the repeat at the 3′ end. In certain embodiments, targeting by the effector proteins described herein may require the lack of homology between the crRNA tag and the target 5′ flanking sequence. This requirement may be similar to that described further in Samai et al. “Co-transcriptional DNA and RNA Cleavage during Type III CRISPR-Cas Immunity” Cell 161, 1164-1174, May 21, 2015, where the requirement is thought to distinguish between bona fide targets on invading nucleic acids from the CRISPR array itself, and where the presence of repeat sequences will lead to full homology with the crRNA tag and prevent autoimmunity.

In certain embodiments, Cas13 effector protein is engineered and can comprise one or more mutations that reduce or eliminate nuclease activity, thereby reducing or eliminating RNA interfering activity. Mutations can also be made at neighboring residues, e.g., at amino acids near those that participate in the nuclease activity. In some embodiments, one or more putative catalytic nuclease domains are inactivated, and the effector protein complex lacks cleavage activity and functions as an RNA binding complex. In a preferred embodiment, the resulting RNA binding complex may be linked with one or more functional domains as described herein.

In certain embodiments of the invention, the guide RNA or mature crRNA comprises, consists essentially of, or consists of a direct repeat sequence and a guide sequence or spacer sequence. In certain embodiments, the guide RNA or mature crRNA comprises, consists essentially of, or consists of a direct repeat sequence linked to a guide sequence or spacer sequence. In preferred embodiments of the invention, the mature crRNA comprises a stem loop or an optimized stem loop structure or an optimized secondary structure. In preferred embodiments the mature crRNA comprises a stem loop or an optimized stem loop structure in the direct repeat sequence, wherein the stem loop or optimized stem loop structure is important for cleavage activity. In certain embodiments, the mature crRNA preferably comprises a single stem loop. In certain embodiments, the direct repeat sequence preferably comprises a single stem loop. In certain embodiments, the cleavage activity of the effector protein complex is modified by introducing mutations that affect the stem loop RNA duplex structure. In preferred embodiments, mutations which maintain the RNA duplex of the stem loop may be introduced, whereby the cleavage activity of the effector protein complex is maintained. In other preferred embodiments, mutations which disrupt the RNA duplex structure of the stem loop may be introduced, whereby the cleavage activity of the effector protein complex is completely abolished.

The CRISPR system as provided herein can make use of a crRNA or analogous polynucleotide comprising a guide sequence, wherein the polynucleotide is an RNA, a DNA or a mixture of RNA and DNA, and/or wherein the polynucleotide comprises one or more nucleotide analogs. The sequence can comprise any structure, including but not limited to a structure of a native crRNA, such as a bulge, a hairpin or a stem loop structure. In certain embodiments, the polynucleotide comprising the guide sequence forms a duplex with a second polynucleotide sequence which can be an RNA or a DNA sequence.

The present disclosure also provides cells, tissues, organisms comprising the engineered CRISPR-Cas protein, the CRISPR-Cas systems, the polynucleotides encoding one or more components of the CRISPR-Cas systems, and/or vectors comprising the polynucleotides. The invention also provides for the nucleotide sequence encoding the effector protein being codon optimized for expression in a eukaryote or eukaryotic cell in any of the herein described methods or compositions. In an embodiment of the invention, the codon optimized effector protein is any Cas13 effector protein discussed herein and is codon optimized for operability in a eukaryotic cell or organism, e.g., such cell or organism as elsewhere herein mentioned, for instance, without limitation, a yeast cell, or a mammalian cell or organism, including a mouse cell, a rat cell, and a human cell or non-human eukaryote organism, e.g., plant.

In a further aspect, the invention provides a eukaryotic cell comprising a modified target locus of interest, wherein the target locus of interest has been modified according to in any of the herein described methods. A further aspect provides a cell line of said cell. Another aspect provides a multicellular organism comprising one or more said cells.

In certain embodiments, the modification of the target locus of interest may result in: the eukaryotic cell comprising altered expression of at least one gene product; the eukaryotic cell comprising altered expression of at least one gene product, wherein the expression of the at least one gene product is increased; the eukaryotic cell comprising altered expression of at least one gene product, wherein the expression of the at least one gene product is decreased; or the eukaryotic cell comprising an edited genome.

In certain embodiments, the eukaryotic cell may be a mammalian cell or a human cell.

In further embodiments, the non-naturally occurring or engineered compositions, the vector systems, or the delivery systems as described in the present specification may be used for: site-specific gene knockout; site-specific genome editing; RNA sequence-specific interference; or multiplexed genome engineering.

Also provided is a gene product from the cell, the cell line, or the organism as described herein. In certain embodiments, the amount of gene product expressed may be greater than or less than the amount of gene product from a cell that does not have altered expression or edited genome. In certain embodiments, the gene product may be altered in comparison with the gene product from a cell that does not have altered expression or edited genome.

In another aspect, the invention provides a method for identifying novel nucleic acid modifying effectors, comprising: identifying putative nucleic acid modifying loci from a set of nucleic acid sequences encoding the putative nucleic acid modifying enzyme loci that are within a defined distance from a conserved genomic element of the loci, that comprise at least one protein above a defined size limit, or both; grouping the identified putative nucleic acid modifying loci into subsets comprising homologous proteins; identifying a final set of candidate nucleic acid modifying loci by selecting nucleic acid modifying loci from one or more subsets based on one or more of the following; subsets comprising loci with putative effector proteins with low domain homology matches to known protein domains relative to loci in other subsets, subsets comprising putative proteins with minimal distances to the conserved genomic element relative to loci in other subsets, subsets with loci comprising large effector proteins having a same orientations as putative adjacent accessory proteins relative to large effector proteins in other subsets, subset comprising putative effector proteins with lower existing nucleic acid modifying classifications relative to other loci, subsets comprising loci with a lower proximity to known nucleic acid modifying loci relative to other subsets, and total number of candidate loci in each subset.

In one embodiment, the set of nucleic acid sequences is obtained from a genomic or metagenomic database, such as a genomic or metagenomic database comprising prokaryotic genomic or metagenomic sequences.

In one embodiment, the defined distance from the conserved genomic element is between 1 kb and 25 kb.

In one embodiment, the conserved genomic element comprises a repetitive element, such as a CRISPR array. In a specific embodiment, the defined distance from the conserved genomic element is within 10 kb of the CRISPR array.

In one embodiment, the defined size limit of a protein comprised within the putative nucleic acid modifying (effector) locus is greater than 200 amino acids, or more particularly, the defined size limit is greater than 700 amino acids. In one embodiment, the putative nucleic acid modifying locus is between 900 to 1800 amino acids.

In one embodiment, the conserved genomic elements are identified using a repeat or pattern finding analysis of the set of nucleic acids, such as PILER-CR.

In one embodiment, the grouping step of the method described herein is based, at least in part, on results of a domain homology search or an HHpred protein domain homology search.

In one embodiment, the defined threshold is a BLAST nearest-neighbor cut-off value of 0 to 1e-7.

In one embodiment, the method described herein further comprises a filtering step that includes only loci with putative proteins between 900 and 1800 amino acids.

In one embodiment, the method described herein further comprises experimental validation of the nucleic acid modifying function of the candidate nucleic acid modifying effectors comprising generating a set of nucleic acid constructs encoding the nucleic acid modifying effectors and performing one or more biochemical validation assays, such as through the use of PFS validation in bacterial colonies, in vitro cleavage assays, the Surveyor method, experiments in mammalian cells, PFS validation, or a combination thereof.

In one embodiment, the method described herein further comprises preparing a non-naturally occurring or engineered composition comprising one or more proteins from the identified nucleic acid modifying loci.

In one embodiment, the identified loci comprise a Class 2 CRISPR effector, or the identified loci lack Cas1 or Cas2, or the identified loci comprise a single effector.

In one embodiment, the single large effector protein is greater than 900, or greater than 1100 amino acids in length, or comprises at least one HEPN domain.

In one embodiment, the at least one HEPN domain is near a N- or C-terminus of the effector protein, or is located in an interior position of the effector protein.

In one embodiment, the single large effector protein comprises a HEPN domain at the N- and C-terminus and two HEPN domains internal to the protein.

In one embodiment, the identified loci further comprise one or two small putative accessory proteins within 2 kb to 10 kb of the CRISPR array.

In one embodiment, a small accessory protein is less than 700 amino acids. In one embodiment, the small accessory protein is from 50 to 300 amino acids in length.

In one embodiment, the small accessory protein comprises multiple predicted transmembrane domains, or comprises four predicted transmembrane domains, or comprises at least one HEPN domain.

In one embodiment, the small accessory protein comprises at least one HEPN domain and at least one transmembrane domain.

In one embodiment, the loci comprise no additional proteins out to 25 kb from the CRISPR array.

In one embodiment, the CRISPR array comprises direct repeat sequences comprising about 36 nucleotides in length. In a specific embodiment, the direct repeat comprises a GTTG/GUUG at the 5′ end that is reverse complementary to a CAAC at the 3′ end.

In one embodiment, the CRISPR array comprises spacer sequences comprising about 30 nucleotides in length.

In one embodiment, the identified loci lack a small accessory protein.

The invention provides a method of identifying novel CRISPR effectors, comprising: a) identifying sequences in a genomic or metagenomic database encoding a CRISPR array; b) identifying one or more Open Reading Frames (ORFs) in said selected sequences within 10 kb of the CRISPR array; c) selecting loci based on the presence of a putative CRISPR effector protein between 900-1800 amino acids in size, d) selecting loci encoding a putative accessory protein of 50-300 amino acids; and e) identifying loci encoding a putative CRISPR effector and CRISPR accessory proteins and optionally classifying them based on structure analysis.

In one embodiment, the CRISPR effector is a Type VI CRISPR effector. In an embodiment, step (a) comprises i) comparing sequences in a genomic and/or metagenomic database with at least one pre-identified seed sequence that encodes a CRISPR array, and selecting sequences comprising said seed sequence; or ii) identifying CRISPR arrays based on a CRISPR algorithm.

In an embodiment, step (d) comprises identifying nuclease domains. In an embodiment, step (d) comprises identifying RuvC, HPN, and/or HEPN domains.

In an embodiment, no ORF encoding Cas1 or Cas2 is present within 10 kb of the CRISPR array

In an embodiment, an ORF in step (b) encodes a putative accessory protein of 50-300 amino acids.

In an embodiment, putative novel CRISPR effectors obtained in step (d) are used as seed sequences for further comparing genomic and/or metagenomics sequences and subsequent selecting loci of interest as described in steps a) to d) of claim 1. In an embodiment, the pre-identified seed sequence is obtained by a method comprising: (a) identifying CRISPR motifs in a genomic or metagenomic database, (b) extracting multiple features in said identified CRISPR motifs, (c) classifying the CRISPR loci using unsupervised learning, (d) identifying conserved locus elements based on said classification, and (e) selecting therefrom a putative CRISPR effector suitable as seed sequence.

In an embodiment, the features include protein elements, repeat structure, repeat sequence, spacer sequence and spacer mapping. In an embodiment, the genomic and metagenomic databases are bacterial and/or archaeal genomes. In an embodiment, the genomic and metagenomic sequences are obtained from the Ensembl and/or NCBI genome databases. In an embodiment, the structure analysis in step (d) is based on secondary structure prediction and/or sequence alignments. In an embodiment, step (d) is achieved by clustering of the remaining loci based on the proteins they encode and manual curation of the obtained clusters. n another aspect, the disclosure provides a mutated Cas13 protein comprising one or more mutations of amino acids, wherein the amino acids: interact with a guide RNA that forms a complex with the mutated Cas 13 protein; or are in a HEPN active site, a lid domain which is a domain that caps the 3′ end of the crRNA with two beta hairpins, a helical domain, selected from a helical 1 or a helical 2 domain, an inter-domain linker (IDL) domain, or a bridge helix domain of the engineered Cas 13 protein. In certain embodiments the helical domain 1 is helical domain 1-1, 1-2 or 1-3. In embodiments helical domain 2 is helical domain 2-1 or 2-2. In one aspect, the engineered Cas13 protein has a higher protease activity or polynucleotide-binding capability compared with a naturally-occurring counterpart Cas13 protein.

In another aspect, the disclosure provides a method of altering activity of a Cas13 protein, comprising: identifying one or more candidate amino acids in the Cas13 protein based on a three-dimensional structure of at least a portion of the Cas 13 protein, wherein the one or more candidate amino acids interact with a guide RNA that forms a complex with the Cas13 protein, or are in a HEPN active site, an inter-domain linker domain, or a bridge helix domain of the Cas13 protein; and mutating the one or more candidate amino acids thereby generating a mutated Cas13 protein, wherein activity the mutated Cas13 protein is different than the Cas13 protein.

### Example Cas13 Proteins and Orthologs

In some examples, Cas13 proteins are Cas13a, e.g., those of SEQ ID NOs. 1-1321. In some examples, Cas13 proteins are Cas13b, e.g., those of SEQ ID NOs. 1324-2770. In some examples, Cas13 proteins are Cas13c, e.g., those of SEQ ID NOs. 2773-2797. In some examples, Cas13 proteins are Cas13d, e.g., those of SEQ ID NOs. 2798-4092.

In some embodiments, the Cas13 proteins include orthologs and homologs of the example Cas13s herein. The systems and compositions may comprise orthologs and homologs of the small Cas proteins. The terms “ortholog” and “homolog” are well known in the art. By means of further guidance, a “homolog” of a protein as used herein is a protein of the same species which performs the same or a similar function as the protein it is a homolog thereof. Homologous proteins may but need not be structurally related, or are only partially structurally related. An “ortholog” of a protein as used herein is a protein of a different species which performs the same or a similar function as the protein it is an ortholog of. Orthologous proteins may but need not be structurally related, or are only partially structurally related. In particular embodiments, the homolog or ortholog of a Cas13 protein as referred to herein has a sequence homology or identity of at least 60%, preferably at least 70%, preferably at least 80%, more preferably at least 85%, even more preferably at least 90%, such as for instance at least 95% with a Cas13 effector protein set forth in SEQ ID NOs. 1-4092, 4102-5203, and 5260-5265 herein.

It has been found that a number of Cas13 orthologs are characterized by common motifs. Accordingly, in particular embodiments, the Cas13 protein is a protein comprising a sequence having at least 70% sequence identity with one or more of the sequences consisting of DKHXFGAFLNLARHN (SEQ ID NO: 4093), GLLFFVSLFLDK (SEQ ID NO: 4094), SKIXGFK (SEQ ID NO: 4095), DMLNELXRCP (SEQ ID NO: 4096), RXZDRFPYFALRYXD (SEQ ID NO: 4097) and LRFQVBLGXY (SEQ ID NO: 4098). In further particular embodiments, the Cas13 protein comprises a sequence having at least 70% sequence identity at least 2, 3, 4, 5 or all 6 of these sequences. In further particular embodiments, the sequence identity with these sequences is at least 75%, 80%, 85%, 90%, 95% or 100%. In further particular embodiments, the Cas13 protein is a protein comprising a sequence having 100% sequence identity with GLLFFVSLFL (SEQ ID NO: 4099) and RHQXRFPYF (SEQ ID NO: 4100). In further particular embodiments, the Cas13 is a Cas13b effector protein comprising a sequence having 100% sequence identity with RHQDRFPY (SEQ ID NO: 4101).

In particular embodiments, the Cas13 protein is a Cas13 protein having at least 65%, preferably at least 70%, 75%, 80%, 85%, 90%, 95% or more sequence identity with a Cas13b protein from Prevotella buccae, Porphyromonas gingivales, Prevotella saccharolytica, or Riemerella anatipestifer. In further particular embodiments, the Cas13b effector is selected from the Cas13b protein from Bacteroides pyogenes, Prevotella sp. MA2016, Riemerella anatipestifer, Porphyromonas gulae, Porphyromonas gingivalis, and Porphyromonas sp.COT-052OH4946.

It will be appreciated that Cas13 proteins that can be within the invention can include a chimeric enzyme comprising a fragment of a Cas13 enzyme of multiple orthologs. Examples of such orthologs are described elsewhere herein. A chimeric enzyme may comprise a fragment of the Cas13 proteins and a fragment from another CRISPR enzyme, such as an ortholog of a Cas13 enzyme of an organism which includes but is not limited to Bergeyella, Prevotella, Porphyromonas, Bacteroides, Alistipes, Riemerella, Myroides, Flavobacterium, Capnocytophaga, Chryseobacterium, Phaeodactylibacter, Paludibacter or Psychroflexus.

In some embodiments, the systems herein also encompass a functional variant of the effector protein or a homolog or an ortholog thereof. A “functional variant” of a protein as used herein refers to a variant of such protein which retains at least partially the activity of that protein. Functional variants may include mutants (which may be insertion, deletion, or replacement mutants), including polymorphs, etc. Also included within functional variants are fusion products of such protein with another, usually unrelated, nucleic acid, protein, polypeptide or peptide. Functional variants may be naturally occurring or may be man-made. In an embodiment, nucleic acid molecule(s) encoding the Cas13 RNA-targeting effector proteins, or an ortholog or homolog thereof, may be codon-optimized for expression in an eukaryotic cell. A eukaryote can be as herein discussed. Nucleic acid molecule(s) can be engineered or non-naturally occurring.

In an embodiment, the Cas13 protein or an ortholog or homolog thereof, may comprise one or more mutations. The mutations may be artificially introduced mutations and may include but are not limited to one or more mutations in a catalytic domain, e.g., one or more mutations are introduced into one or more of the HEPN domains.

In certain example embodiments, the Cas13 effector protein is from an organism. In certain example embodiments, the Cas13 effector protein is from an organism selected from Bergeyella zoohelcum, Prevotella intermedia, Prevotella buccae, Porphyromonas gingivalis, Bacteroides pyogenes, Alistipes sp. ZOR0009, Prevotella sp. MA2016, Riemerella anatipestifer, Prevotella aurantiaca, Prevotella saccharolytica, Myroides odoratimimus CCUG 10230, Capnocytophaga canimorsus, Porphyromonas gulae, Prevotella sp. P5-125, Flavobacterium branchiophilum, Myroides odoratimimus, Flavobacterium columnare, or Porphyromonas sp. COT-052 OH4946. In another embodiment, the one or more guide RNAs are designed to bind to one or more target RNA sequences that are diagnostic for a disease state.

### Small Cas Proteins and Orthologs

The systems and compositions herein comprise Cas proteins that are relatively small. The Cas proteins may have less than 1000, less than 950, less than 900, less than 850, less than 800, less than 750, less than 700, less than 650, less than 600, less than 550, less than 500, less than 450, less than 400, less than 350, or less than 300 amino acids in size. In some examples, the Cas proteins have less than 900 amino acids in size. In some examples, the Cas proteins have less than 850 amino acids in size. In some examples, the Cas proteins have less than 800 amino acids in size. In some examples, the Cas proteins have less than 750 amino acids in size. In some examples, the Cas proteins have less than 700 amino acids in size.

In some embodiments, the Cas proteins are a subgroup of Type VI-B1 Cas proteins with no auxiliary proteins. In some examples, the CRISPR-array in loci of the Cas proteins are processed and no other non-coding RNAs (ncRNAs) are present. In some examples, the Cas proteins are Cas13b-t.

In some embodiments, the small Cas proteins are small Cas 13a. Examples of small Cas13a are shown in Table 1 below.

TABLE 1

| Accession | |
|---|---|
| No. | Sequences |
| IMG_3300008161_ | MKITKIDGVSHYKEKEKGVLKGKDILNGKIEKIVKKRYDATIESKIYKEFIKLRKNRIEQNNEKSILKLIK |
| 3 | LNIDKNEKEIKTLLLNKFKIKEKNKKYDKYMLDENKLDNDIKIYESVESLYFLIKEIYLGQNNKKWNIS |
| SEQ ID NO: | KIDLEKIMEEDNNLIMLGYKLKKNITENDYPYLYSDKNGQESTSVYKLLKKLIEENKDRNQDIRKSQEY |
| 4102 | EKIRKNFEEYKNRKINLLVKSIKNNKINIQYINNEIKSHNNSREENIIKFFKKMIEEKNEPILKDKLKLFKL |
| | EVFFDEEFLEEIKKLLDSDDFDKSYNKKISELRGKIFNRIREEIKNNKNRDELENIYFLELKKYIENNLSH |
| | KKEKDKNNNNTGEEKSKELYLKFLKKVLFIDDNNRISIEKLKSRIDDNFKNLLIQHVIEYGKIKYYVEN |
| | DDYIRNIVKNGELKLETKDLEYIKTKETLIRKMAVLVSFATNSYYNLFGRTENNIPTQEISDDLLLGKIE |
| | NEIYIKGERNRRYVFKEKMLNYFFYSEIFGDNKIVEVLNAISSSIYNIRNGVNHFDKMILGKYNNGLDLK |
| | DSDTIKDYFNFKKKEIQQDLKDRFISNNLQYYYTENEIKKYFEKYKFEILKTKASFAPNFKRILIKGENLS |
| | ISESNNSYEFFKAYSESSDKNTEYNEFMKTRNFLLKELYYNNFYTEFLNNKAKFNEAVKKVKKNKKKR |
| | AENKGRAAGKSYDMIENYNFSDNIPEYISYIHKSEMERIEINTEKNRRDTSKHIRDFIEEIFLEGFIEYLDN |
| | NNFKFLKKRNEVDKEREEIVRNLNIQIEGLDILNENDSEILNLYLFFNMIDNKRISEFRNDMIKYKQFLA |
| | KRQNIDSKFLKIDIEKIEAIIEFVIITKEKLEILEGETKEQKKR |
| UPJI01.1 | MCMKITKIDGVSHYKEKEKGVLKAKGVLNEEIQKIVKKRYDKTIESKIYKEFIKLRKNRIEQNNEKSILE |
| SEQ ID NO: | LIKSNIDKNEKEIKTLLWKNFKIKEKNKKYDKYILDENKLDNDIKIYESAESLYFFIKEVYLGENNKKW |
| 4103 | NISKIDLEKIMEEDSDLIMLGYKLKKNIKEDDYPYLYRDKNGQESTSVYELLKKLIEENKDRNQDIRESE |
| | EYRKIQKEFKEYKNRKINLLVKSILNNKVNIKYNTNNNSLEDSNSKREKEIIEFFKKMIEEKNKPILKDK |
| | LELFRLEVFFDEEFLEEIKKLLDSDDSDKSDNKKIAELRGKIFSRIREKIKEDKNRGILKNIYFLELRKYIE |
| | NNLSHKKEKNKNKNNNIGEEKSKELYLEFLKKVLFIDDNNRISIEKLKSRIDDNFKNLLIQHVIEYGKIK |
| | YYVENDDYIRNIVKNGELKLETENLEYIRIRETLIRKMAVLVSFAANSFYNLFENITSDILTANINLDSDV |
| | IKIGNNRLKEKFLNYFFYSEEISDKEDFLKALKDSIYNVRNGVNHFDKMILGKYNNGLDLKDSNTIKDY |
| | FNFKKKEIQQDLKDRFISNNLQYYYTENEIKKYFEKYKFEILKTKASFAPNFKRILIKGENLSISESNNSY |
| | EFFKAYSESSDKNTEYNEFMKTRNFLLKELYYNNFYTEFLNNKAKFKDFKDKVAFALVSPFLVSSMIAI |
| | SPVLFIESLIED |
| IMG_3300008271_ | MKITKIDGISHKKYIKEGKLVKSTSEENKTDERLSELLIIRLDTYIKNPDNASEEENRIRRENLKEFFSNK |
| 2 | VLYLKDGILYLKDRREKNQLQNKNYSEEDISEYDLKNKNSFSVLKKILLNEDINSEELEIFRNDFEKKLD |
| SEQ ID NO: | KINSLKYSLEENRANYQKINENNIKKVEGKSKRNIFYNYYKDSAKRNDYINNIQEAFDKLYKKEDIENL |
| 4104 | FFLIENSKKHEKYKIRECYHKIIGRKNDKENFATIIYEEIQNVNNMKELIEKVPNVSELKKSQVFYKYYL |
| | NKEKLNDENIKYVFCHFVEIEMSKLLKNYVYKKPSNISNDKVKRIFEYQSLKKLIENKLLNKLDTYVRN |
| | CGKYSFYLQDGEIATSNFIVENRQNEAFLRNIIGVSSAAYFSLRNILETENENDITGRIKGKTVKNNKGEE |
| | KYISGEIDKLYDNNKQNEVKKNLKMFYSYDFNMNSKKEIEDFFSNIDEAIRQSKKYRGSH |
| IMG_3300007713 | MKITKIDGISHKKYIKEGKLVKSTSEENKTDERLSELLIIRLDTYIKNPDNASEEENRIRRENLKEFFSNK |
| SEQ ID NO: | VLYLKDGILYLKDRREKNQLQNKNYSEEDISEYDLKNKNSFSVLKKILLNEDINSEELEIFRNDFEKKLD |
| 4105 | KINSLKYSLEENRANYQKINENNIKKVEGKSKRNIFYNYYKDSAKRNDYINNIQEAFDKLYKKEDIENL |
| | FFLIENSKKHEKYKIRECYHKIIGRKNDKENFATIIYEEIQNVNNMKELIEKVPNVSELKKSQVFYKYYL |
| | NKEKLNDENIKYVFCHFVEIEMSKLLKNYVYKKPSNISNDKVKRIFEYQSLKKLIENKLLNKLDTYVRN |
| | CGKYSFYLQDGEIATSNFIVENRQNEAFLRNIIGVSSAAYFSLRNILETENENDITGRIKGKTVKNNKGEE |
| | KYISGEIDKLYDNNKQNEVKKNLKMFYSYDFNMNSKKEIEDFFSNIDEAIRQSKKYRGSH |
| UPJG01.1 | LYMKITKIDGISHKKYIKEGKLVKSTSEENKTDERLSELLTIRLDTYIKNPDNASEEENRIRRENLKEFFS |
| SEQ ID NO: | NKVLYLKDGILYLKDRREKNQLQNKNYSEQDISEYDLKNKNNFLVLKKILLNEDINSEELEIFRNDFEK |
| 4106 | KLDKINSLKYSLEENKANYQKINENNIEKVEGKSKRNIFYNYYKDSAKRNDYINNIQEAFDKLYKKEDI |
| | ENLFFFIENSKKHEKYKIRECYHKIIGRKNDKENFSKIIYEEIQNVNNMKELIEKVPNVSELKKSQVFYK |
| | YYLNKEKLNDENIKYVFCHFVEIEMSKLLKNYVYKKPSNISNDKVKRIFEYQSLKKLIENKLLNKLDTY |
| | VRNCGKYSFYLQDGEIATSNFIVGNRQNEAFLRNIIGVSSAAYFSLRNILETENENDITGRIKGKTVKNN |
| | KGEEKYISGEIDKLYDNNKQNEVKKNLKMFYSYDFNMNSKKEIEDFFSNIDEAISSIRHGIVHFNLELEG |
| | KDIFTFKNIVPSQISKKMFQDEINEKKLKLKIFRQLNSANVFRYLEKYKILNYLKRTRFEFVNKNIPFVPS |
| | FTKLYSRIDDLKNSLCIYWKIPKANDNNKTKEITDAQIYLLKNIYYGDKVLNEADPKSFKSLSKISYGLG |
| | KDKNNLYF |
| IMG_3300011928 | MKITKIDGISHKKYIKEGKLVKSTSEENKTDERLSELLTIRLDTYIKNPDNASEEENRIRRENLKEFFSNK |
| SEQ ID NO: | VLYLKDGILYLKDRREKNQLQNKNYSEEDISEYDLKNKNSFLVLKKILLNEDINSEELEIFRNDFEKKFN |
| 4107 | KINSLKYSLEENKANYQKINENNIKKVEGKSKRNIFYNYYKDSAKRNDYINNIQEAFDKLYKKEDIENL |
| | FFLIENSKKHEKYKIRECYHKIIGRKNDKENFSKIIYEEIQNVNNMKELIEKVPNVSELKKSQVFYKYYL |
| | NKEKLNDENIKYVFCHFVEIEMSKLLKNYVYKKPSNISNDKVKRIFEYQSLKKLIENKLLNKLDTYVRN |
| | CGKYSFYLQDGEIATSDFIVGNRQNEAFLRNIIGVSSTAYFSLRNILETENENDITGRIKGKTVKNNKGEE |
| | KYISGEIDKLYDNNKQNEVKKNLKMFYSYDFNMNRKKEIEDFFSNIDEAISSIRHGIVHFNLELEGKDIF |
| | TFKNIVPSQISKKMFQNEINEKKLKLKIFRQLNSANVFRYLEKYKILNYLNRTRFEFVNKNIPFVPSFTKL |
| | YSRIDDLKNSLCIYWKIPKANDNNKTKEITDAQIYLLKNIYYGEFLNYFMSNNGNFFEITKEIIELNKND |
| | KRNLKTGFYKLQKFENLQEKTPKEYLANIQSLYMINAGNQDEEEKDTYIDFIQKIFLKGFMTYLANNG |
| | RLSLIYIGSDEETNTSLAEKKQEFDKFLKKYEQNNNIEIPHEINEFVREIKLGKILKYTESLNIFYLILKLLN |
| | HKEFTNLKGSLEKYQSANKEEAFSDQLELINLLNLDNNRVTEDFELEADEIGKFLDFNGNKVKDNKEL |
| | KKFDTNKIYFDGENIIKHRAFY |
| IMG_3300009393 | MKITKIDGISHKKYIKEGKLVKSTSEENKTDERLSELLTIRLDTYIKNPNNASEEENRIRRENLKEFFSNK |
| SEQ ID NO: | VLYLKDGILYLKDRREKNQLQNKNYSEEDISEYDLKNKNNFLVLKKILLNEDINSEELEIFRNDFEKKL |
| 4108 | DKINSLKYSLEENKANYQKINENNIEKVEGKSKRNIFYNYYKDSAKRNDYINNIQEAFDKLYKKEDIEN |
| | LFFLIENSKKHEKYKIRECYHKIIGRKNDKENFATIIYEEIQNVNNMKELIEKVPNVSELKKSQVFYKYY |
| | LNKEKLNDENIKYVFCHFVEIEMSKLLKNYVYKKPSNISNDKVKRIFEYQSLKKLIENKLLNKLDTYVR |
| | NCGKYSFYLQDGEIAISDFIVGNRQNEAFLRNIIGVSSTAYFSLRNILETENENDITGRIKGKTVKNNKGG |
| | EKYNSRQHLKDKYVAERLSIE |
| IMG_3300011936 | MKVTKVDGISHKKYIEEGKLVKSTSEENRTSERLSELLSIRLDIYIKNPDNASEEENRIRRENLKKFFSNK |
| SEQ ID NO: | VLYLKDSVLYLKNRNEKNAVQDKNYSEEDISEYDLKNKNSFSVLKKILLNEDINSEELEIFRKDVEAKL |
| 4109 | NKINSLKYSFEENKANYQKINENNVEKVGGKSKRNIIYDYYRESAKRNDYINNVQEAFDKLYKKEDIE |
| | KLFFLIENSKKHEKYKIRECYHKIIGRKNDKENFAKIIYEEIQNVNNIKELIEKVPNMSELKKSQVFYKYY |
| | LDKEELNDKNIKYAFCHFVEIEMSKLLKNYVYKKPSNISNDKVKRIFEYQNLKKLIENKLLNKLDTYVR |
| | NCGKYNYYLQVGEIATSDFIARNRQNEAFLRNIIGVSSVAYFSLRNILETENENDITGRMRGKTVKNNK |
| | GEEKYVSGEVDKIYNENKQNEVKENLKMFYSYDFNMDNKNEIEDFFANIDEAISSIRHGIVHFNLELEG |
| | KDIFAFKNIVPSEISKKMFQNEINEKKLKLKIFRQLNSANVFRYLEKYKILNYLKRTRFEFVNKNIPFVPS |
| | FTKLYSRIDDLKNSLGIYWKTPKTNDDNKTKEIIDAQIYLLKNIYYGEFLNYFMSNNGNFFEISREIIELN |
| | KNDKRNLKTGFYKLQKFEDIQEKTPKEYLANIQSLYMINAGNQD |
| IMG_3300006462 | MKVTKVDGISHKKYIEEGKLVKSTSEENRTSERLSELLSIRLDIYIKNPDNASEEENRIRRENLKKFFSNK |
| SEQ ID NO: | VLHLKDSVLYLKNRKEKNAVQDKNYSEEDISEYDLKNKNSFSVLKKILLNEDINSEELEIFRKDVEAKL |
| 4110 | NKINSLKYSFEENKANYQKINENNVEKVGGKSKRNIIYDYYRESAKRNDYINNVQEAFDKLYKKEDIE |
| | KLFFLIENSKKHEKYKIRECYHKIIGRKNDKENFAKIIYEEIQNVNNIKELIEKVPDMSELKKSQVFYKYY |
| | LDKEELNDKNIKYAFCHFVEIEMSQLLKNYVYKRLSNISNDKIKRIFEYQNLKKLIENKLLNKLDTYVR |
| | NCGKYNYYLQDGEIATSDFIARNRQNEAFLRNIIGVSSVAYFSLRNILETENENDITGRMRGKTVKNNK |
| | GEEKYVSGEVDKIYNENKQNEVKENLKMFYSYDFNMDNKNEIEDFFANIDEAISSIRHGIVHFNLELEG |
| | KDIFAFKNIVPSEISKKMFQNEINEKKLKLKIFRQLNSANVFRYLEKYKILNYLKRTRFEFVNKNIPFVPS |
| | FTKLYSRIDDLKNSLGIYWKTPKTNDDNKTKEIIDAQIYLLKNIYYGEFLNYFMSNNGNFFEISKEIIELN |
| | KNDKRNLKTGFYKLQKFEDIQEKTPKEYLANIQSLYMINAGNQDEEEKDTYIDFIQKIFLKGFMTYLAN |
| | NGRLSLMYIGNDEQINTSLAGKKQEFDKFLKKYEQNNNIEIPHEINEFVREIKLGKILKYTESLNMFYLIL |
| | KLLNHKELTNLKGSLEKYQSANKEETFSDELELINLLNLDNNRVTEDFELEANEIGKFLDFNGNKIKDR |
| | KELKKFDTNKIYFDGENIIKHRAFYNIKKYG |
| IMG_3300008161 | MKVTKVDGISHKKYIEEGKLVKSTSEENRTSERLSELLSIRLDIYIKNPDNASEEENRIRRENLKKFFSNK |
| SEQ ID NO: | VLHLKDSVLYLKNRKEKNAVQDKNYSEEDISEYDLKNKNSFSVLKKILLNEDINSEELEIFRKDVEAKL |
| 4111 | NKINSLKYSFEENKANYQKINENNIEKVEGKSKRNIIYDYYRESAKRNDYINNVQEAFDKLYKKEDIET |
| | LFFLIENSKKHEKYKIRECYHKIIGRKNDKENFAKIIYEEIQNVNNIKELIEKVPDMSELKKSQVFYKYYL |
| | DKEELNDENIKYVFCHFVEIEMSKLLKNYVYKKPSNISNDKVKRIFEYQSLKKLIENKLLNKLDTYVRN |
| | CGKYNYYLQDGEIATSDFIARNRQNEAFLRNIIGVSSVAYFSLRNILETENENDITGRMRGKTVKNNKG |
| | EEKYVSGEVDKIYNENKQNEVKENLKMFYSYDFNMDNKNEIEDFFANIDEAISSIRHGIVHFNLELEGK |
| | DIFAFKNIAPSEISKKMFQNEINEKKLKLKIFRQLNSANVFRYLEKYKILNYLKRTRFEFVNKNIPFVPSF |
| | TKLYSRIDDLKNSLGIYWKTPKTNDDNKTKEIIDAQIYLLKNIYYGEFLNYFMSNNGNFFEISREIIELNK |
| | NDKRNLKTGFYKLQKFEDIQEKTPKEYLANIQSLYMINAGNQDEEEKDTYIDFIQKIFLKGFMTYLANN |
| | GRLSLIYIGSDE |
| IMG_3300008486 | MKVTKVDGISHKKYIEEGKLVKSTSEENRTSERLSELLSIRLDIYIKNPDNASEEENRIRRENLKKFFSNK |
| SEQ ID NO: | VLHLKDSVLYLKNRKEKNAVQDKNYSEEDISEYDLKNKNSFSVLKKILLNEDINSEELEIFRKDVEAKL |
| 4112 | NKINSLKYSFEENKANYQKINENNIEKVEGKSKRNIIYDYYRESAKRNDYINNVQEAFDKLYKKEDIEK |
| | LFFLIENSKKHEKYKIREYYHKIIGRKNDKENFAKIIYEEIQNVNNIKELIEKVPDMSELKKSQVFYKYYL |
| | DKEELNDENIKYVFCHFVEIEMSKLLKNYVYKKPSNISNDKVKRIFEYQSLKKLIENKLLNKLDTYVRN |
| | CGKYNYYLQDGEIATSDFIARNRQNEAFLRNIIGVSSVAYFSLRNILETENENDITGRMRGKTVKNNKG |
| | EEKYVSGEVDKIYNENKQNEVKENLKMFYSYDFNMDNKNEIEDFFANIDEAISSIRHGIVHFNLELEGK |
| | DIFAFKNIAPSEISKKMFQNEINEKKLKLKIFRQLNSANVFRYLEKYKILNYLKRTRFEFVNKNIPFVPSF |
| | TKLYSRIDDLKNSLGIYWKTPKTNDDNKTKEIIDAQIYLLKNIYYGEFLNYFMSNNGNFFEISREIIELNK |
| | NDKRNLKTGFYKLQKFEDIQEKTPKEYLANIQSLYMINAGNQDEEEKDTYIDFIQKIFLKGFMTYLANN |
| | GRLSLIYIGSDE |
| IMG_3300006254_ | MKVTKVDGISHKKYIEEGKLVKSTSEENRTSERLSELLSIRLDIYIKNPDNASEEENRIRRENLKKFFSNK |
| 2 | VLHLKDSVLYLKNRNEKNAVQDKNYSEEDISEYDLKNKNSFSVLKKILLNEDINSEELEIFRKDVEVKL |
| SEQ ID NO: | NKINSLKYSFEENKANYQKINENNVEKVGGKSKRNIIYDYYRESAKRNDYINNVQEAFDKLYKKEDIE |
| 4113 | NLFFLIENSKKNEKYKIRECYHKIIGRKNDKENFAKIIYEEIQNVNNIKELIEKVPDMSELKKSQVFYKYY |
| | LDKEELNDKNIKYAFCHFVEIEMSKLLKNYVYKKPSNISNDKVKRIFEYQSLKKLIENKLLNKLDTYVR |
| | NCGKYNYYLQDGEIATSDFIARNRQNEAFLRNIIGVSSAAYFSLRNILETENENDITGRMRGKTVKNNK |
| | GEEKYVSGEVDKIYNENKQNEVKENLKMFYSYDFNVDNKNEIEDFFVNIDEAISSIRHGIVHFNLELEG |
| | KDIFAFKNIAPSEISKKMFQNEINEKKLKLKIFRQLNSANVFRYLEKYKILNYLKRTRFEFVNKNIPFVPS |
| | FTKLYSRIDDLKNSLGIYWKTPKTNDDNKTKEIIDAQIYLLKNIYYGEFLNYFMSNNGNFFEISKEIIELN |
| | KNDKRNLKTGFYKLQKFEDIQEKTPKE |
| UPJS01.1 | MKVTKVDGISHKKYIEEGKLVKSTSEENRTSERLSELLSIRLDIYIKNPDNASEEENRIRRENLKKFFSNK |
| SEQ ID NO: | VLHLKDSVLYLKNRNEKNAVQDKNYSEEDISEYDLKNKNSFSVLKKILLNEDINSEELEIFRKDVEAKL |
| 4114 | NKINSLKYSFEENKANYQKINENNVEKVGGKSKRNIIYDYYRESAKRNDYINNVQEAFDKLYKKEDIE |
| | KLFFLIENSKKHEKYKIRECYHKIIGRKNDKENFAKIIYEEIQNVNNIKELIEKVPDMSELKKSQVFYKYY |
| | LDKEELNDKNIKYAFCHFVEIEMSKLLKNYVYKKPSNISNDKVKRIFEYQSLKKLIENKLLNKLDTYVR |
| | NCGKYNYYLQDGEIATSDFIARNRQNEAFLRNIIGVSSVAYFSLRNILETENENDITGRMRGKTVKNNK |
| | GEEKYVSGEVDKIYNENKQNEVKENLKMFYSYDFNVDNKNEIEDFFVNIDEAISSIRHGIVHFNLELEG |
| | KDIFAFKNIAPSEISKKMFQNEINEKKLKLKIFRQLNSANVFRYLEKDRILDYLRSTRFEFVNKNIPFVPSF |
| | TKLYDRIDDLKNSLDIYWKIPKTKDDIKTKEITDAQIYLLKNIYYGKFLDYFMSRNGNFFKISREVIKLN |
| | KNDKRNLKTGFYKLQKFEDIQEKTPKEYLANIQSLYMINAGNQDEEEKDTYIDFIQKIFLKGFMTYLAN |
| | NGRLSLMYIGNDEQINTSLAGKK |
| IMG_3300014815 | MKVTKVDGISHKKYIEEGKLVKSTSEENRTSERLSELLSIRLDIYIKNPDNASEEENRIRRENLKKFFSNK |
| SEQ ID NO: | VLHLKDSVLYLKNRNEKNTVQDKNYSEEDISEYDLKNKNSFSVLKKILLNEDINSEELEIFRKDVEAKL |
| 4115 | NKINSLKYSFEENKANYQKINENNVEKVGGKSKRNIIYDYYRESAKRNDYINNVQEAFDKLYKKEDIE |
| | NLFFLIENSKKHEKYKIRECYHKIIGRKNDKENFAKIIYEEIQNVNNIKELIEKVPDMSELKKSQVFYKYY |
| | LDKEELNDKNIKYAFCHFVEIEMSKLLKNYVYKKPSNISNDKVKRIFEYQSLKKLIENKLLNKLDTYVR |
| | NCGKYNYYLQDGEIATSDFIARNRQNEAFLRNIIGVSSVAYFSLRNILETENENDITGR |
| IMG_3300007794 | MKVTKVDGISHKKYIEEGKLVKSTSEENRTSERLSELLSIRLDIYIKNPDNASEEENRIRRENLKKFFSNK |
| SEQ ID NO: | VLHLKDSVLYLKNRKEKNAVQDKNYSEEDISEYDLKNKNSFLVLKKILLNEDINSEELEIFRKDVEAKL |
| 4116 | NKINSLKYSFEENKANYQKINENNVEKVGGKSKRNIIYDYYRESAKHNDYINNVQEAFDKLYKKEDIE |
| | NLFFLIENSKKHEKYKIRECYHKIIGRKNDKENFAKIIYEEIQNVNNIKELIEKVPNMSELKKSQVFYKYY |
| | LDKEELNDKNIKYAFCHFVEIEMSKLLKNYVYKKPSNISNDKVKRIFEYQSLKKLIENKLLNKLDTYVR |
| | NCGKYNYYLQDGEIATSDFIARNRQNEAFLRNIIGVSSVAYFSLRNILETENENDITGRMRGKTVKNNK |
| | GEEKYVSGEVDKIYNENKQNEVKENLKMFYSYDFNMDNKNEIEDFFANIDEAISSIRHGIVHFNLELEG |
| | KDIFAFKNIAPSEISKKIFQNEINEKKLKLKIFRQLNSANVFNFYEKDVIIKYLKNTKFNFVNKNIPFVPSF |
| | TKLYNKIDDLRNTLKFSWKIPKDKEEKDAQIYLLKNIYYGEFLNKFVKNSKDFFKITDEVIKINKQINQK |
| | TGYYKYQKFENIEKTVPVEYLAIIQSRDMINNQDKEEKNTYIDFVQQIFLKGFIDYLNKNNLKYIENNN |
| | NN |
| UPUO01.1 | MKVTKVDGISHKKYIEEGKLVKSTSEENRTSERLSELLSIRLDIYIKNPDNASEEENRIRRENLKKFFSNK |
| SEQ ID NO: | VLHLKDSVLYLKNRKEKNAVQDKNYSEEDISEYDLKNKNSFSVLKKILLNEDVNSEELEIFRKDVEAK |
| 4117 | LNKINSLKYSFEENKANYQKINENNVEKVGGKSKRNIIYDYYRESAKHNDYINNVQEAFDKLYKKEDI |
| | ENLFFLIENSKKHEKYKIRECYHKIIGRKNDKENFAKIIYEEIQNVNNIKELIEKVPDMSELKKSQVFYKY |
| | YLDKEELNDENIKYAFCHFVEIEMSKLLKNYVYKKPSNISNDKVKRIFEYQSLKKLIENKLLNKLDTYV |
| | RNCGKYNYYLQDGEIATSDFIARNRQNEAFLRNIIGVSSVAYFSLRNILETENENDITGRMRGKTVKNN |
| | KGEEKYVSGEVDKIYNENKQNEVKENLKMFYSYDFNMDNKNEIEDFFANIDEAISSIRHGIVHFNLELE |
| | GKDIFAFKNIVPSEISKKMFQNEINEKKLKLKIFKQLNSANVFNFYEKDVIIKYLKNTKFNFVNKNIPFVP |
| | SFTKLYNKIDDLRNTLKFSWKIPKDKEEKDAQIYLLKNIYYGEFLNKFVKNSKDFFKITDEVIKINKQRN |
| | QKTGYYKYQKFENIEKTVPVEYLAIIQSRDMINNQDKEEKNTYIDFVQQIFLKGFIDYLNKNNLKYIEN |
| | NNNNDNNDIFSKIKIKKDNKEKY |
| UPWA01.1 | MKVTKVDGISHKKYIEEGKLVKSTSEENRTSERLSELLSIRLDIYIKNPDNASEEENRIRRENLKKFFSNK |
| SEQ ID NO: | VLHLKDSVLYLKNRNEKNAVQDKNYSEEDISEYDLKNKNSFSVLKKILLNEDINSEELEIFRKDVEAKL |
| 4118 | NKINSLKYSFEENKANYQKINENNVEKVGGKSKRNIIYDYYRESAKHNDYINNVQEAFDKLYKKEDIE |
| | KLFFFIENSKKHEKYKIRECYHKIIGRKNDKENFAKIIYEEIQNVNNIKELIEKVPDMSELKKSQVFYKYY |
| | LDKEELNDKNIKYAFCHFVEIEMSKLLKNYVYKKPSNISNDKVKRIFEYQSLKKLIENKLLNKLDTYVR |
| | NCGKYNYYLQDGEIATSDFIARNRQNEAFLRNIIGVSSVAYFSLRNILETENENDITGRMRGKTVKNNK |
| | GEEKYVSGEVDKIYNENKQNEVKENLKMFYSYDFNVDNKNEIEDFFVNIDEAISSIRHGIVHFNLELEG |
| | KDIFAFKNIAPSEISKKMFQNEINEKKLKLKIFRQLNSANVFNFYEKDVIIKYLKNTKFNFVNKNIPFVPS |
| | FTKLYNKIDDLRNTLKFSWKIPKDKEEKDAQIYLLKNIYYGEFLNKFVKNSKDFFKITDEVIKINKQRNQ |
| | KTGYYKYQKFENIEKTVPVEYLAIIQSRDTINNQDKEEKNTYIDFVOOIFLKGFIDY |
| UPKY01.1 | MKVTKVDGISHKKYIEEGKLVKSTSEENRTSERLSELLSIRLDIYIKNPDNASEEENRIRRENLKKFFSNK |
| SEQ ID NO: | VLHLKDSVLYLKNRKEKNAVQDKNYSEEDISEYDLKNKNSFSVLKKILLNEDINSEELEIFRKDVEAKL |
| 4119 | NKINSLKYSFEKNKANYQKINENNIEKVGGKSKRNIIYDYYRESAKRNDYINNVQEAFDKLYKKEDIEK |
| | LFFLIENSKKHEKYKIRECYHKIIGRKNDKENFAKIIYEEIQNVNNIKELIEKVPDMSELKKSQVFYKYYL |
| | DKEELNDKNIKYAFCHFVEIEMSQLLKNYVYKRLSNISNDKIKRIFEYQNLKKLIENKLLNKLDTYVRN |
| | CGKYNYYLQDGEIATSDFIAGNRQNEAFLRNIIGVSSVAYFSLRNILETENENDITGRMRGKTVKNNKG |
| | EEKYVSGEVDKIYNENKQNEVKENLKMFYSYDFNMDNKNEIEDFFANIDEAISSIRHGIVHFNLELEGK |
| | DIFAFKNIVPSEISKKMFQNEINEKKLKLKIFRQLNSANVFNFYEKDVIIKYLKNTKFNFVNKNIPFVPSF |
| | TKLYNKIDDLRNTLKFSWKIPKDKEEKDAQIYLLKNIYYGEFLNKFVKNSKDFFKITDEVIKINKQRNQ |
| | KTGYYKYQKFENIEKTVPVEYLAIIQSRDMINNQDKEEKNTYIDFVQQIFLKGFIDYLNKNNLKYIENN |
| | NNNDIFSRIKIKKDSKER |
| UPAK01.1 | MKVTKVDGISHKKYIEEGKLVKSTSEENRTSERLSELLSIRLDIYIKNPDNASEEENRIRRENLKKFFSNK |
| SEQ ID NO: | VLHLKDSVLYLKNRNEKNAVQDKNYSEEDISEYDLKNKNSFLVLKKILLNGDINSEELEIFRNDFEKKL |
| 4120 | DKLNSLKYSLEENKANYQKINENNIKKVEGKSKRNIFYNYYKDSAKRNDYINNIQEAFDKLYKKEDIE |
| | NLFFLIENSKKHEKYKIRECYHKIIGRKNDKENFAKIIYEEIQNVNNIKELIEKVPDMSELKKSQVFYKYY |
| | LDKEELNDENVKYVFCHFVEIEMSKLLKNYVYKKPSNISNDKVKRIFEYQSLKKLIENKLLNKLDTYV |
| | RNCGKYNYYLQDGEIATSDFIAGNRQNEAFLRNIIGVSSVAYFSLRNIEETENENDITGRMRGKTVKNN |
| | KGEEKYVSGEVDKIYNENKQNEVKENLKMFYSYDFNMDNKNEIEDFFANIDEAISSIRHGIVHFNLELE |
| | GKDIFAFKNIAPSEISKKMFQNEINEKKLKLKIFRQLNSANVFRYLEKYKILNYLKRTRFEFVNKNIPFVP |
| | SFTKLYSRIDDLKNSLGIYWKTPKTNDDNKTKEIIDAQIYLLKNIYYGEFLNYFMSNNGNFFEISREIIEL |
| | NKNDKRNLKTGFYKLQKFEDIQEKTPKEYLANIQSLYMINAGNQDEEEKDTYIDFIQKIFLKGFMTYLA |
| | NNGRLSLIYIGSDEETNTSLAEKKQEFDKFLKKYEQNNNIEIPHEINEFVREIKLGKILKY |
| IMG_ | MKVTKVDGISHKKYIEEGKLVKSTSEENRTGERLSELLSIRLDIYIKNPDNASEEENRIRRENLKKFFSNK |
| 3300008635 | VLHLKDSVLYLKNRKEKNAVQDKNYSEEDISEYDLKNKNSFSVLKKILLNEDINSEELEIFRKDVEAKL |
| SEQ ID NO: | NKINSLKYSFEENKANYQKINENNVEKVVGKSKRNIIYDYYRESAKRNDYINNVQEAFDKLYKKEDIE |
| 4121 | KLFFLIENSKKHEKYKIRECYHKIIGRKNDKENFAKIIYEEIQNVNNMKELIEKVPDMSELKKSQVFYKY |
| | YLDKEELNDENVKYVFCHFVEIEMSQLLKNYVYKRLSNISNDKIKRIFEYQNLKKLIENKLLNKLDTYV |
| | RNCGKYNYYLQVGEIATSDFIARNRQNEAFLRNIIGVSSVAYFSLRNILETENENDITGRMRGKTVKNN |
| | KGEEKYVSGEVDKIYNENKKNEVKENLKMFYSYDFNMDNKNEIEDFFANIDEAISSIRHGIVHFNLELE |
| | GKDIFTFKNIVPSQISKKMFQDEINEKKLKLKIFKQLNSANVFNFYEKDVIIKYLKNTFLNLYSFSRPSIL |
| UPVU01.1 | LYMKITKIDGISHKKYIKEGKLVKSTSEENKTDERLSELLTIRLDTYIKNPDNASEEENRIRRENLKEFFS |
| SEQ ID NO: | NKVLYLKDGILYLKDRREKNQLQNKNYSEEDISEYDLKNKNNFLVLKKILLNEDINSEELEIFRNDFEK |
| 4122 | KLDKINSLKYSLEENKANYQKINENNIEKVEGKSKRNIFYNYYKDSAKRNDYINNVQEAFDKLYKKED |
| | IEKLFFLIENSKKHEKYKIRECYHKIIGRKNDKENFAKIIYEEIQNVNNIKELIEKVPDMSELKKSQVFYK |
| | YYLDKEELNDENIKYAFCHFVEIEMSKLLKNYVYKKPSNISNDKVKRIFEYQSLKKLIENKLLNKLDTY |
| | VRNCGKYNYYLQVGEIATSDFIARNRQNEAFLRNIIGVSSVAYFSLRNILETENENDITGRMRGKTVKN |
| | NKGEEKYVFGEVDKIYNENKQNEVKENLKMFYSYDFNMNSKKEIEDFFSNIDEAISSIRHGIVHFNLEL |
| | EGKDIFAFKNIAPSEISKKMFQNEINEKKLKLKIFRQLNSANVFNFYEKDVIIKYLKNTKFNFVNKNIPFV |
| | PSFTKLYNKIDDLRNTLKFSWKIPKDKEEKDAQIYLLKNIYYGEFLNKFVKNSKDFFKITDEVIKINKQR |
| | NQKTGYYKYQKFENIEKTVPVEYLAIIQSRDMINNQDKEEKNTYIDFVQQIFLKGFIDYLNKNNLKYIE |
| | NN |
| UPUV01.1 | LYMKITKIDGISHKKYIKEGKLVKSTSEENKTDERLSELLTIRLDTYIKNPDNASEEEKRIRRETLKEFFS |
| SEQ ID NO: | NKVLHLKDGILYLKDRREKNQLQNKNYSEQDISEYDLKNKNSFSVLKKILLNEDINSEELEIFRNDFEK |
| 4123 | KLDKINSLKYSLEENKANYQKINENNIKKVEGKSKRNIFYNYYKDSAKRNDYINNIQEAFDKLYKKEDI |
| | ENLFFLIENSKKHEKYKIRECYHKIIGRKNDKENFATIIYEEIQNVNNMKELIEKVPDMSELKKSQVFYK |
| | YYLDKEELNDENIKYAFCHFVEIEMSKLLKNYVYKKPSNISNDKVKRIFEYQSLKKLIENKLLNKLDTY |
| | VRNCGKYNYYLQDGEIATSDFIAGNRQNEAFLRNIIGVSSVAYFSLRNILETENKNDITGKIRGKTRIESK |
| | TGEEKYIPGEVDQIYYENKQNEVKNKLKMFYGYDFDMDNKKEIEDFFANIDEAISSIRHGIVHFNLELE |
| | GKDIFAFKNIVPSEISKKMFQNEINEKKLKLKIFRQLNSANVFNFYEKDVIIKYLKNTKFNFVNKNIPFVP |
| | SFTKLYNKIDDLRNTLKFSWKIPKDKEEKDAQIYLLKNIYYGEFLNKFVKNSKDFFKITDEVIKINKQRN |
| | QKTGYYKYQKFENIEKTVPVEYLAIIQSRDMINNQDKEEKNTYI |
| UPDS01.1_2 | MKVTKVDGISHKKYIEEGKLVKSTSEENRTGERLSELLSIRLDIYIKNPDNASEEENRIRRENLKKFFSNK |
| SEQ ID NO: | VLHLKDSVLYLKNRKEKNAVQDKNYSEEDISEYDLKNKNSFSVLKKILLNEDVNSEELEIFRKDVEAK |
| 4124 | LNKINSLKYSFKENKANYQKINENNVEKVGGKSKRNIIYDYYRESAKRDAYVSNVKEAFDKLYKEEDI |
| | AKLVLKIENLTKLEKYKIREFYHEIIGRKNDKENFAKIIYEEIQNVNNMKELIEKVPDMSELKKSQVFYK |
| | YYLDKEELNDKNIKYAFCHFVEIEMSKLLKNYVYKKPSNISNDKVKRIFEYQSLKKLIENKLLNKLDTY |
| | VRNCGKYNYYLQDGEIATSDFIAGNRQNEAFLRNIIGVSSVAYFSLRNILETENKDDITGKIRGKTRIESK |
| | TGEEKYIPGEVDQIYYENKQNEVKNKLKMFYGYDFDMDNKKEIEDFFANIDEAISSIRHGIVHFNLELE |
| | GKDIFAFKNIVPSEISKKMFQNEINEKKLKLKIFRQLNSANVFNFYEKDVIIKYLKNTKFNFVNKNIPFVP |
| | SFTKLYNKIDDLRNTLKFSWKIPKVKEEKDAQIYLLKNIYYGEFLNKFVKNSKDFFKITDEVIKINKQRN |
| | QKT |
| UPXI01.1 | MKITKIDGISHKKYIKEGKLVKSTSEENKTDERLSELLTIRLDTYIKNPDNASEEENRIRRENLKEFFSNK |
| SEQ ID NO: | VLHLKDGILYLKDRREKNQLQNKNYSEEDISEYDLKNKNNFLVLKKILLNEDINSEELEIFRNDFEKKL |
| 4125 | DKINSLKYSLEENKANYQKINENNIKKVEGKSKRNIFYNYYKDSAKRNDYINNIQEAFDKLYKKEDIEN |
| | LFFLIENSKKHEKYKIRECYHKIIGRKNDKENFSKIIYEEIQNVNNMKELIEKVPNVSELKKSQVFYKYY |
| | LNKEKLNDENIKYVFCHFVEIEMSKLLKNYVYKKPSNISNDKVKRIFEYQSLKKLIENKLLNKLDTYVR |
| | NCGKYNYYLQDGEIATSDFIARNRQNEAFLRNIIGVSSVAYFSLRNILETENKDDITGKIRGKTRIDSKTR |
| | EEKYIPGEVDQIYYENKQNEVKNKLKMFYGYDFDMDNKKEIEDFFANIDEAISSIRHGIVHFNLELEGK |
| | DIFAFKNIAPSEISKKMFQNEINEKKLKLKIFKQLNSANVFRYLEKDRILDYLRSTRFEFVNKNIPFVPSFT |
| | KLYDRIDDLKISLNIYWKTPKTNDDIKTKEITDAQIYLLKNIYYGKFLDKFLNEENGIFISIKDKIIELNRN |
| | QNKRTGFYKLEKFETLKANTPTEYLEKLQSLHKINYDREKIEKWIAAGDQNLCVLDAELI |
| IMG_ | MKVTKVGGISHKKYTSEGRLVKSESEENRTDERLSALLNMRLDMYIKNPSSTETKENQKRIGKLKKFF |
| 3300006317 | SNKMVYLKDNTLSLKNGKKENIDREYSETDISEYDVRDSKNFAVLKKIYLNENVNSEELEVFRKDIKK |
| SEQ ID NO: | KLNKINSLKYSFEKNKANYQKINENNIEKVGGKSKRNIIYDYYRESAKRNDYINNVQEAFDKLYKKEDI |
| 4126 | EKLFFLIENSKKHEKYKIRECYHKIIGRKNDKENFAKIIYEEIQNVNNIKELIEKVPNMSELKKSQVFYKY |
| | YLDKEELNDKNIKYAFCHFVEIEMSKLLKNYVYKKPSNISNDKVKRIFEYQSLKKLIENKLLNKLDTYV |
| | RNCGKYNYYLQDGEIATSDFIAGNRQNEAFLRNIIGVSSVAYFSLRNILETKNKDDITGKIRGKTRIESKT |
| | GEEKYIPGEVDQIYYENKQNEVKNKLKMFYGYDFDMDNKKEIEDFFANIDEAISSIRHGIVHFNLELEG |
| | KDIFAFKNIVPSEISKKMFQNEINEKKLKLKIFRQLNSANVFNFYEKDVIIKYLKNTKFNFVNKNIPFVPS |
| | FTKLYNKIDDLRNTLKFSWKIPKDKEEKDAQIYLLKNIYYGKFLDYFMSRNGNFFEISREVIKLNKNNK |
| | KNVKTGFYKLEKFENLEARSPKEYLAKVQSLYIINVANQDEEEKNTYIDFIQKVFLKGFIDYLNKNNLK |
| | YIENNNNNDIFSRIKIKKDSKERYDKILKNYEKNNRNKEIPHEINEFVREIKLGKILKYTESLNMFYLILK |
| | SLNHKEL |
| ODUT01.1 | MKVTKVGGISHKKYTSEGRLVKSESEENRTDERLSALLNMRLDMYIKNPSNTETKENKKRIGKLKKFF |
| SEQ ID NO: | SNKMVYLKDNTLSLKNGKKENIDREYSETDISEYDVRDSKNFAVLKKIYLNENVNSEELEVFRKDIKK |
| 4127 | KLNKINSLKYSFEKNKANYQKINENNIEKVEGKSKRNIIYDYYRESAKRDAYVSNVKEAFDKLYKEEDI |
| | AKLVLKIENLTKLEKSKIREFYHEIIGRKNDKENFAKIIYEEIQNVNNMKELIEKVPDMSELKKSQVFYK |
| | YYLDKEELNDENVKYVFCHFVEIEMSKLLKNYVYKKPSNISNDKVKRIFEYQSLKKLIENKLLNKLDT |
| | YVRNCGKYNYYLQDGEIATSDFIARNRQNEAFLRNIIGVSSVAYFSLRNILETENKDDITGKMRGKTRIE |
| | SKTGEEKYIPGEVDQIYYENKQNEVKNKLKMFYGYDFDMDNKKEIEDFFANIDEAISSIRHGIVHFNLD |
| | LDGKDIFAFKNIVPSEISKKMFQNEINEKKLKLKIFRQLNSANVFRYLEKDRILDYLRSTRFEFVNKNIPF |
| | VPSFTKLYDRIDDLKISLNIYWKTPKTNDDIKTKEITDAQIYLLKNIYYGKFLDYFMSRNGNFFEISREVI |
| | KLNKIGRAV |
| IMG_ | MTYLANNGRLSLIYIGSDEETNTSLAGKKQEFDKFLKKYEQNNNIEIPHEINEFVREIKLGKILKYTESLN |
| 3300011936_2 | MFYLILKLLNHKELTNLKGSLEKYQSANKEEAFSDQLELINLLNLDNNRVIEDFELEADEIGKFLDFNG |
| SEQ ID NO: | NKIKDRKELKKFDTNKIYFDGENIINHRAFYNIKKYGMLNLLEKIADKAKYKISLKELKEYSNKKNEIE |
| 4128 | KNYTMQQNLHRKYARPKKDEKFNDEDYKEYEKAIGNIQKYTHLKNKVEFNELNLLQGLLLKILHRLV |
| | GYTSIWERDLRFRLKGEFPENQYIEEIFNFDNSKNVKYKSGQIVEKYINFYKELYKDNVEKRSIYSDKK |
| | VKKLKQEKKDLYIRNYIAHFNYIPHAEISLLEVLENLRKLLSYDRKLKNAVMKSVVNILKEYGFVAKF |
| | KIGADKKIGIQTLESEKIVHLKNLKKKKLMTDRNSKELCELVKVMFEYKMEEKKSEN |
| UPUH01.1_2 | MSELKKSQVFYKYYLDKEELNDENIKYAFCHFVEIEMSKLLKNYVYKKPSNISNDKVKRIFEYQNLKK |
| SEQ ID NO: | LIENKLLNKLDTYVRNCGKYNYYLQVGEIATSDFIARNRQNEAFLRNIIGVSSVAYFSLRNILETENEND |
| 4129 | ITGRMRGKTVKNNKGEEKYVSGEVDKIYNENKQNEVKENLKMFYSYDFNMDNKNEIEDFFANIDEAI |
| | SSIRHGIVHFNLELEGKDIFAFKNIVPSEISKKMFQNEINEKKLKLKIFRQLNSANVFRYLEKYKILNYLK |
| | RTRFEFVNKNIPFVPSFTKLYSRIDDLKNSLGIYWKTPKTNDDNKTKEIIDAQIYLLKNIYYGEFLNYFM |
| | SNNGNFFEISREIIELNKNDKRNLKTGFYKLQKFEDIQEKTPKEYLANIQSLYMINAGNQDEEEKDTYID |
| | FIQKIFLKGFMTYLANNGRLSLIYIGSDEETNTSLAEKKKEFDKFLKKYEQNNNIEIPHEINEFVREIKLG |
| | KILKYTESLNMFYLILKLLNHKELTNLKGSLEKYQSANKEEAFSDQLELINLLNLDNNRVTEDFELEAD |
| | EIGKFLDFNGNKIKDRKELKKFDTNKIYFDGENIIKHRAFYNIKKYGMLNLLEKIADKAKYKISLKELKE |
| | YSNKKNEIEKNYTMQQNLHRKYARPKKDEKFTDEDYKKYEKAIRNIQQYTHLKNKVEFNELNLLQGL |
| | LLKILHRLVGYTSIWERDLRFRLKGEFPENQYIEEIFNFDNSKNVKYKSGQIVEKYINFYKELYKDNVEK |
| | RSIYSDKKVKELKKEKKDLYIRNYIAHFNYIPNAEVSLLEVLENLRKLLSYDRKLKNAVMKSVVDILKE |
| | YGFVATFKIGADKKIGIQTLESEKIVHLKNLKKKKLMTDRNSEELCELVKVMFEYKMKEKKSEN |
| UPII01.1 | MDLLNRAIYLQDGEIATSDFIARNRQNEAFLRNIIGVSSVAYFSLRNILETENENDITGRMRGKTVKNNK |
| SEQ ID NO: | GEEKYVSGEVDKIYNENKQNEVKENLKMFYSYDFNMDNKNEIEDFFANIDEAISSIRHGIVHFNLELEG |
| 4130 | KDIFAFKNIAPSEISKKMFQNEINEKKLKLKIFRQLNSANVFRYLEKYKILNYLKRTRFEFVNKNIPFVPS |
| | FTKLYSRIDDLKNSLGIYWKTPKTNDDNKTKEIIDAQIYLLKNIYYGEFLNYFMSNNGNFFEISREIIELN |
| | KNDKRNLKTGFYKLQKFEDIQEKTPKEYLANIQSLYMINAGNQDEEEKDTYIDFIQKIFLKGFMTYLAN |
| | NGRLSLMYIGNDEQINTSLAGKKQEFDKFLKKYEQNNNIEIPHEINEFVREIKLGKILKYTESLNMFYLIL |
| | KLLLYLHLLKFLHYMHHIKMLIILYHILQKVL |
| UPDS01.1 | MSWAERLSGLLSGLNIVHSLPPFRRELWALCAIMATMTMTKRRSRTQTTPENNPRHPLAMPATVSIEM |
| SEQ ID NO: | WERFSFYGMQAILAYYLYYATTDGGLGLERAQATTLLGAYGASVYLCTLAGGWIGDRLIGTERTLLT |
| 4131 | GCIALMVGHLSLSTLSGGAGATFGLALIAIGSGFVKTAYIDFVQQIFLKGFIDYLNKNNLKYIENNNNND |
| | IFSRIKIKKDSKERYDKILKNYEKNNRNKEIPYEINEFVREIKLGKILKYTERLNMFYLILKLLNHKELTN |
| | LKGSLEKYQSANKEEAFSDQLELINLLNLDNNRVTEDFELEVNEIGKFLDFNRNKIKDRKELKKFDTKK |
| | lYFDGENIIKHRAFYNIKKYGMLNLLEKIADKAKYKISIEELRNYSNKKNEIEKNHTTQENLHRKYARPR |
| | KDEKFTDEDYENYKRAIKNIEEYTHLKNKVEFNELNLLQGLLLRILHRLVGYTSIWERDLRFRLKGEFP |
| | ENQYIEEIFNFNNKQNVKYKSGQIVEKYIKFYKELYQNDEMKINKYSSANIKVLKQEKKDLYIRNYIAH |
| | FNYIPHAEISLLEVLENLRKLLSYDRKLKNAVMKSVVDILKEYDFVVKFKIGADKKIEIQSLKSEEIVHL |
| | KKLKLKDNDKKKEPIKTYRNSKELCKLVKVMFEYKYGRKKF |
| UPUT01.1 | MINLYKYMGMKSVKNIEDRLFAVIQKIMNESIEASYISQYDNFNKLKNISNKIIAVLDAGDYIDNAKVIR |
| SEQ ID NO: | DLDRLIYKYEIFTMIPNLDNKHIVSIQSDQNSFCEFINKSIVDHLNYDVSINIPYIILPYCESFCANSVYILS |
| 4132 | YCNKIVELTIDEYKLICTELYKYNIDIKKLIKCFFSYQSRVTTNTCFVYFPLDMDIENTVYCQLKDKITVS |
| | VFIGNEIFKNKLYYNSFYFLGSKSEYKKFFHVYKSKYIKCISYKNLIDRIKKFDNVFYNYNIAQEIDLLLL |
| | EVKKFYINSLNRLSNILKGIKTDLLRIQDDKLKEQLQYYYEYKQIEYDELSISKNKFCKFYSEILNYILNN |
| | GLSNDYYDINLLNLDNNRVTEDFELEANEIGKFLDFNGNKIKDRKELKKFDTNKIYFDGENIIKHRAFY |
| | NIKKYGMLNLLEKISDEAKYKISIEELKNYSNKKNEIEKNHTTQENLHRKYARPRKDEKFTDEDYKKY |
| | EKAIRNIQQYTHLKNKVEFNELNLLQSLLLRILHRLVGYTSIWERDLRFRLKGEFPENQYIEEIFNFDNSK |
| | NVKYKNGQIVEKYINFYKELYKDDTEKISIYSDKKVKELKKEKKDLYIRNYIAHFNYIPHAEISLLEVLE |
| | NLRKLLSYDRKLKNAVMKSVVDILKEYGFVVKFKIGADKKIEIQSLKSEEIVHLKKLKLKDNDKKKEPI |
| | KTYRNSKELCKLVKVMFEYKMEEKSSEK |
| UPAU01.1 | MSFSVKKLFSNLFLSVVLEGNECIFFGQVFRNGKLLKTINAKFTDINIDSIDEKIIKYIEEQEKAYFGVYV |
| SEQ ID NO: | SVFFNDDSQGALPSVSFDEYKKFNINTKNLTSLIMQDSWSIYANLNAIKKYKNLQKELEKNDFYKIQEK |
| 4133 | IHRKYNQKPNLISRTENKKDFNDYKKAIENIQNYTQLKNKIEFNDLNLLQGLLFRILHRLAGYTSLWER |
| | DLQFKLKGEFPEDKYIDEIFNSDRNNNQKYKSGGIAYKYVDFLIEKEEGKRAGKNKVKKRSEKEGSFII |
| | RNYIAHFNYIPDAEKSILEMLEELRELLKYDRKLKNAVMKSIKDIFKEYGFIVEFGISHESNSKKIKVLNV |
| | ESEKIKHLKNNGLVTTRNSKDLCKLVKVMLEYKKS |
| UPKT01.1 | MKIDTYEKSYNGTHSLYNLIKLGRNRYTIELRIYEEITEEEEKFFKKLFKEEIKKYENLQKELEKNDFYKI |
| SEQ ID NO: | QENIHRKYNQKPNLILRTENKKDFNDYKKAIENIQNYTQLKNKIEFNDLNLLQSLLFRILHRLAGYTSL |
| 4134 | WERDLQFKLKGEFPEDKYIDEIFNFDNSKNEKYKNGAIVFKYVDFLIEKKEGKRAGTKKINKKSEEKGL |
| | EIRNYIAHFNYIPDATKSILEILEELRNLLKYDRKLKNAVMKSIKDIFKEYGFIVEFTISHTKNGKKIKVCS |
| | VKSEKIKHLKNNELITTRNSEDLCDLVKIMLEYKKLQK |
| UPGE01.1 | LFKILVLPLRKIDFKFAQRPDLLLANSKYSQDEIKKYLENVKIKFTNKNIPFVPEFSKLYNRIENLKGDNA |
| SEQ ID NO: | LKLGQNIIVPKRKEAKDSQLYLLKNIYYGEFVEKFVNDNENFVKIAEEIIEINKTAGTNEKTKFYKLEKF |
| 4135 | KTLSADTPTKYLKKLQSLHKINYDKEKVEESKDVYVDFVQKIFLKGFVNYLQNSNTLRVLNLLKLDKD |
| | EVITTKKSFYDENLKKWEKMGSDLSELPTDIYEFVKKIKVDEINYSDRMSIFYLLLKLLNHKELTSLRG |
| | NLEKYESMNKNNIYEEELEIINLVSLDNNKVQTNFELEADEVGKFLNTATPIKKITQLNDFSDIYADRQN |
| | VIKYRSFYNLKKYSVLDLIAEIVGKGNAKIKEEEIKKYENLQNELEEKGFYRIQENIHKKYNKNPKMIN |
| | KKDLEDYDNAIRKIEEYTQMKNKLEFNDLNLLQSIMFRILHRMAGYTSIWERDLQFKLRGEYPEKSTEI |
| | SEMFTGRIIDNYKNFIKPLKEINKSLKKPTESERKNKKGMYIRNYIAHFNYIPYAELSILEMLERLRALLS |
| | YDRKLKNAVMKSVTDILKEYGFEVEFKISHPEEINQNNNEIVETIEVKKVESVKIEHLKNAKFKKDKKLI |
| | TKKNSEELCKLVKVMLEYKKPE |
| QWBZ01.1_ | MGKDVFSFINRNISFVPSFTKIYNRVQDLANSLEIKEWKIPDESEGKDAQIYLLKNIYYGKFLDKFLNEE |
| 2 | NGIFISIKDKIIELNRNQNKRTGFYKLEKFEKIEETNPKKYLEIIQSLYMINIEEIDSEGKNIFLDFIQKIFLK |
| SEQ ID NO: | GFFEFIKNDYNYLLELKKVQDKKNIFDSKMSEYIAGEKTLEDMEEINEIIQDIKITEIDKILNQTDKINCFY |
| 4136 | LLLKLLNYKEITELKGNLEKYQILSKTNVYEKELMLLNIVNLDNNKVKIENFKISAEEIGKFIEKINIEEIN |
| | KNKKIKTFEELRNFEKGENTGEYYNIYSDDKNIKNIRNLYNIKKYGMLDLLEKISEKINYCIKKKDLEEY |
| | SKLRKQLEDEKTNFYKIQEYLHSKYQQKPKKILWKNNKNDYEKYKKSIENIEKYVHLKNKIEFNELNL |
| | LQSLLLKILHRLVGFTSIWERDLRFRLTGEFSDESDVEDIFDHRKRYKGTGGGICKKYDRFINTYTEYKN |
| | NNKMKNVKFDDNTPVRNYIAHFNYLPNPKYSILKMMEKLRKLLDYDRKLKNAVMKSIKDILEEYGFK |
| | AEFIINSDKEIILNLVKSVEIIHLGKEDLKSHRNSEDLCKLVKAMLEYSK |
| IMG_ | MKVTKIDGISHKKYEEKGKLVKINNEKKDITEERFNDIEVKTMELFQKTLDFYVKNYEKCEEQNKERR |
| 3300007646 | EKAKNYFSKVKLIVDNKKIKICNENPEKMEIEDFNEYDVRNRKYFNILNKILNEENRTEEDLEVFENDL |
| SEQ ID NO: | QKKLNQIQSIKNSLEENKAHFKKESINNTTDRVKGNNKKSLFYEYYRNSSKHQEYVNNIFEAFDKLYSN |
| 4137 | SHEDINNLFLEITKDSNDRNIRKIREAYHEILNKNKTEFGEELYKKIQDNISNFDKLLEIEPEIKELTKSQIF |
| | YKYYIDKVSLDGTNVKHCFSHLVEIEVNQLLKNYVYSKRSTNKEKLENIFEYCKLRNLVKNKLVNKLN |
| | SYIRNCGKYNSYINNNDVVNSEKISEIRTKEAFLRSIIGVSSSAYFSLRNILNTDNTQDITNKVDKEVDKL |
| | YQENKKIELEERLKLFFGNYFDINNQQEIEDFLMNIDKIISSIRHEIIHFKMEANAQNIFDFNNINLGNTAK |
| | NIFNNEINEEKIKFKIFKQLNSANVFDYLSNKDITEYMDKVVFSFTNRNVSFVPSFTKIYNRVQDLANSL |
| | EIKEWKIPDESEGKDAQIYLLKNIYYGKFLDKFLNEENGIFISIK |
| IMG_ | MKITKIGGISHKKYEEKGKLIKSNEIEKDVIEERFSNIEAKTTELFSKTLDFYVKNYEKCEEQNKERREK |
| 3300007320_2 | AKNYFSKVKLIVDNKKITIFNENTEKIEIEGFNEYDVRDEKYFNVLNKILKEENCTEEDLEVFENDLQKK |
| SEQ ID NO: | LNQIQSIKNSLEKNKAHFKKESINNTTDRVKGNNKKSLFYEYYRNSSKHQEYVNNIFEAFDKLYSNSHE |
| 4138 | DINNLFLEITKDSNNRNIRKIREVYNEILNKNKTEFGEELYKKIQDNISNFDKLLEIEPEIKELTKSQIFYK |
| | YYIDKVSLDGTNIKHCFSHLVEIEVNQLLKNYVYSKRSTNKEKLENIFEYCKLRNLVKNKLVNKLNNYI |
| | RNCGKYNSYINNNDVVNSEKISEIRTKEAFLRSIIGVSSSAYFSLRNILNTDNTQDITNKVKGEVEKLYQ |
| | ENKKVKLEERLKLFFGNNFDINNQQEIEDFLMNIDKIISNIRHEIIHFKIEANAHSIFDFNNVTLGNKAKNI |
| | FNNEINEERIKFKIFKQLNSANVFDYLSDENITEYMGKVIFSFTNRNIPFVPSFRKIYNRVQDLANSLKIKE |
| | WKISDESEGKDAQIYLLKNIYYGEFLDDFLNEKNEKFIKIKDEIIELNKNQNKITGFYKLEKFEKIEEKNP |
| | KKYLEIIQSLYMINIEEIDNEEKNIFLDFIQKIFLKGF |
| IMG_ | MKITKIGGISHKKYEEKGKLIKSNEIEKDVIEERFSNIEAKTTELFSKTLDFYVKNYEKCEEQNKERREK |
| 3300014038_2 | AKNYFSKVKLIVDNKKITIFNENTEKIEIEGFNEYDVRDEKYFNVLNKILKEENCTEEDLEVFENDLQKK |
| SEQ ID NO: | LNQIQSIKNSLEKNKAHFKKESINNTTDRVKGNNKKSLFYEYYRNSSKHQEYVNNIFEAFDKLYSNSHE |
| 4139 | DINNLFLEITKDSNNRNIRKIREVYNEILNKNKTEFGEELYKKIQDNISNFDKLLEIEPEIKELTKSQIFYK |
| | YYIDKVSLDGTNIKHCFSHLVEIEVNQLLKNYVYSKRSTNKEKLENIFEYCKLRNLVKNKLVNKLNNYI |
| | RNCGKYNSYINNNDVVNSEKISEIRTKEAFLRSIIGVSSSAYFSLRNILNTDNTQDITNKVKGEVEKLYQ |
| | ENKKVKLEERLKLFFGNNFDINNQQEIEDFLMNIDKIISNIRHEIIHFKIEANAHSIFDFNNVTLGNKAKNI |
| | FNNEINEERIKFKIFKQLNSANVFDYLSDENITEYMGKVIFSFTNRNIPFVPSFRKIYNRVQDLANSLKIKE |
| | WKISDESEGKDAQIYLLKNIYYGEFLDDFLNEKNEKFIKIKDEIIELNKNQNKITGFYKLEKFEKIEEKNP |
| | KKYLEIIQSLYMINIEEIDNEEKNIFLDFIQKIFLKG |
| OEEI01.1 | MKITKIGGISHKKYEEKGKLIKSNEIEKDVIEERFSNIEAKTTELFSKTLDFYVKNYEKCEEQNKERREK |
| SEQ ID NO: | AKNYFSKVKLIVDNKKITIFNENTEKIEIEGFNEYDVRDEKYFNVLNKILKEENCTEEDLEVFENDLQKK |
| 4140 | LNQIQSIKNSLEKNKAHFKKESINNTTDRVKGNNKKSLFYEYYRNSSKHQEYVNNIFEAFDKLYSNSHE |
| | DINNLFLEITKDSNNRNIRKIREVYNEILNKNKTEFGEELYKKIQDNISNFDKLLEIEPEIKELTKSQIFYK |
| | YYIDKVSLDGTNIKHCFSHLVEIEVNQLLKNYVYSKRSTNKEKLENIFEYCKLRNLVKNKLVNKLNNYI |
| | RNCGKYNSYINNNDVVNSEKISEIRTKEAFLRSIIGVSSSAYFSLRNILNTDNTQDITNKVKGEVEKLYQ |
| | ENKKVKLEERLKLFFGNNFDINNQQEIEDFLMNIDKIISNIRHEIIHFKIEANAHSIFDFNNVTLGNKAKNI |
| | FNNEINEERIKFKIFKQLNSANVFDYLSDENITEYMGKVIFSFTNRNIPFVPSFRKIYNRVQDLANSLKIKE |
| | WKISDESEGKDAQIYLLKNIYYGEFLDDFLNEKNEKFIKIKDEIIELNKNQNKITGFYKLEKFEKIEEKNP |
| | KKYLEIIQSLYMINIEEIDNEEKNIFLDFIQKIFLK |
| UPKN01.1 | MKITKIGGISHKKYEEKGKLIKSNEIEKDVIEERFSNIEAKTTELFSKTLDFYVKNYEKCEEQNKERREK |
| SEQ ID NO: | AKNYFSKVKLIVDNKKITIFNENTEKIEIEDFNEYDVRNRKYFNVLNKILNGENYTEEDLEVFENDLQK |
| 4141 | KLNQIQSIKNSLEENKAHFKKESINNTTDRVKGNNKKSLFYEYYRNSSKHQEYVNNIFEAFDKLYSNSH |
| | EDINNLFLEITKDSNDRNIRKIREAYHEILNKNKTEFGEELYKKIQDNINNFDKLLEIEPEIKELTKSQIFY |
| | KYYIDKVSLDGTNIKHCFSHLVEIEVNQLLKNYVYSKRSTNKEKLENIFEYCKLRNLVKNKLVNKLNS |
| | YIRNCGKYNSYINNNDVVNSEKISEIRTKEAFLRSIIGVSSSAYFSLRNILNTDNTQDITNKVKGEVEKLY |
| | QENKKVKLEERLKLFFGNNFDINNQQEIEDFLMNIDKIISNIRHEIIHFKIEANAHSIFDFNNVTLGNKAK |
| | NIFNNEINEERIKFKIFKQLNSANVFDYLSDENITEYMGKVIFSFTNRNIPFVPSFRKIYNRVQDLANSLKI |
| | KEWKKNRK |
| ODUM01.1 | MRGDYMKITKIDGISHKKYKEKGKLIKSNEIEKDVTEERFNDIEVKTTELFQKTLDFYVKNYEKCEEQN |
| SEQ ID NO: | KERREKAKNYFSKVKLIVDNKKITIFNENTEKIEIEGFNEYDVRDEKYFNVLNKILKEENCTEEDLEVFE |
| 4142 | NDLQKKLNQIQSIKNSLEENKAHFKKESVNNTADRVKGNNKKSLFYEYYRNSSKHQEYVNNIFEAFDK |
| | LYSNSHEDMNNLFSAITKDSNDRNIKKIREAYHEILNKNKIEFGEELYKKIQDNINNFDKLLEIEPEIKEL |
| | TKSQIFYKYYIDKVSLDGTNIKHCFSHLVEIEVNQLLKNYVYSKRSTNKEKLENIFEYCKLKNLVKNKL |
| | VNKLNSYIRNCGKYNSYINNNDVVNSEKISEIRTKEAFLRSIIGVSSSAYFSLRNILNTDNTQDITNKVKG |
| | EVEKLYQENKKVKLEERLKLFFGNNFDINNQQEIEDFLMNIDKIISNIRHEIIHFKIEANAHSIFDFNNVTL |
| | GNKAKNIFNNEINEERIKFKIFKQLNSANVFDYLSDENITEYMGKVIFSFTNRNIPFVPSFRKIYNRVQDL |
| | ANSLKIKEWKISDESEGKDAQIYLLKNIYYEEFLDEFLNEENGIFISIKDKIIELNRNQNKRTGFYKLEKFE |
| | KIEEKNPKKYLEIIQSLYMINIEEIDNEEKKIGRAV |
| IMG_ | MRGDYMKITKIDGISHKKYKEKGKLIKSNEIEKDVTEERFNDIEVKTTELFQKTLDFYVKNYEKCEEQN |
| 3300008755 | KERREKAKNYFSKVKLIVDNKKITIFNENTEKIEIEGFNEYDVRDEKYFNVLNKILKEENCTEEDLEVFE |
| SEQ ID NO: | NDLQKKLNQIQSIKNSLEENKAHFKKESVNNTADRVKGNNKKSLFYEYYRNSSKHQEYVNNIFEAFDK |
| 4143 | LYSNSHEDMNNLFSAITKDSNDRNIKKIREAYHEILNKNKIEFGEELYKKIQDNINNFDKLLEIEPEIKEL |
| | TKSQIFYKYYIDKVSLDGTNIKHCFSHLVEIEVNQLLKNYVYSKRSTNKEKLENIFEYCKLKNLVKNKL |
| | VNKLNSYIRNCGKYNSYINNNDVVNSEKISEIRTKEAFLRSIIGVSSSAYFSLRNILNTDNTQDITNKVKG |
| | EVEKLYQENKKVKLEERLKLFFGNNFDINNQQEIEDFLMNIDKIISNIRHEIIHFKIEANAHSIFDFNNVTL |
| | GNKAKNIFNNEINEERIKFKIFKQLNSANVFDYLSDENITEYMGKVIFSFTNRNIPFVPSFRKIYNRVQDL |
| | ANSLKIKEWKISDESEGKDAQIYLLKNIYYEEFLDEFLNEENGIFISIKDKITI |
| IMG_ | MKITKINGISHKKYEEKGKLVKINDEKKNITEERFNDIEAKTTELFQKTLDFYVKNYEKCEDQNKERRE |
| 3300006317_2 | KAKNYFSKVKILVDNKKITICNENTEKMEIEDFNEYDVRNRKFFNVLNKILNRENYTEEDLEVFENDLQ |
| SEQ ID NO: | KRIGRIKSIKNSLEENKAHFKKENVNDNNRVKGNNKKSLFYEYYRVSSKHQEYVDNIFETFDKLYSNS |
| 4144 | HENMNNLFLEITKDSNDRNIRKIREAYHEILNKNKTEFGEELYKKIQDNISNFDKLLEIEPEIKELTKSQIF |
| | YKYYIDKVSLDGTNIKHCFSHLVEIEVNQLLKNYVYSKRSTNKEKLENIFEYCKLRNLVKNKLVNKLN |
| | NYIRNCGKYNSYINNNDVVNSEKISEIRTKEAFLRSIIGVSSSAYFSLRNILNTDNTQDITNKVKGEVEKL |
| | YQENKKVKLEERLKLFFGNNFDINNQQEIEDFLMNIDKIISNIRHEIIHFKIEANAHSIFDFNNVTLGNKA |
| | KNIFNNEINEERIKFKIFKQLNSANVFDYLSDENITEYMGKVIFSFTNRNIPFVPSFRKIYNRVQDLANSLE |
| | SIL |
| IMG_ | MRGGYMKITKIGGISHKKYEEKGKLIKSNEIEKDVIEERFNDIEKKTKELFLKTLDSYVKNYEKCEEQN |
| 3300008481 | KERREKAKNYFSKVKLIIDNEKITICNENTEKMEIEDFNEYDVRNRKYFNVLNKILNGENYTEEDLEVF |
| SEQ ID NO: | ENDLQKKLNQIQSIKNSLEENKAHFKKESINNTTDIVKGNNKKSLFYEYYRNSSKHQEYVNNIFEAFDK |
| 4145 | LYSNSHEDINNLFLEITKDSNDRNIRKIREAYHEILNKNKTEFGEELYKKIQDSISNFDKLLEIEPEIKELT |
| | KSQIFYKYYIDKVNLDETSTKHCFCHLVEIEVNQLLRNYVYSKRNISKEKLKNIFEYCKLKNLIKNKLV |
| | NKLNNYIRNCGKYNGYISNNDVINSEKISEIRTKEAFLRSIIGVSSSAYFSLRNILNTDNTQDITNKVDKE |
| | VDKLYQENKKIELEERLKLFFGNHFDINNQQEIKAFLMNIDKIISSIRHEIIHFKMEANVQNIFDFNNINLG |
| | NKAKNIFSNEINEEKIKFKIFKQLNSANVFDYLSDENITEYMGKAVFSFTNRNIPFVPSFTKIYNKVQDLA |
| | NSLEIKKWKIPNESEGKDAQIYLLKNIYYGKFLDEFLNEENGIFISIKDKIIELNRNQNKRTGFYKLEKFE |
| | KIEETNPKKYLEIIQSLYMINIEEIDSEGKNIFLDFIQKIFLKGFFEFIK |
| IMG_ | VNNIFEAFDKLYSNSHEDINNLFLEITKDSNDRNIRKIREAYHEILNKNKTEFGEELYKKIQDSISNFDKL |
| 3300008743 | LEIEPEIKELTKSQIFYKYYIDKVNLDETSTKHCFCHLVEIEVNQLLRNYVYSKRNISKEKLKNIFEYCKL |
| SEQ ID NO: | KNLIKNKLVNKLNNYIRNCGKYNGYISNNDVINSEKISEIRTKEAFLRSIIGVSSSAYFSLRNILNTDNTQ |
| 4146 | DITNKVDKEVDKLYQENKKIELEERLKLFFGNYFDINNQQEIKVFLMNIDKIISSIRHEIIHFKMEANVQN |
| | IFDFNNINLGNKAKNIFSNEINEEKINVDKDVVVTN |
| IMG_ | MKITKIDGISHKKYKEKGKLIKSNEIEKDITEERFNDIEAKTTELFQKTLDFYVKNYENSEDQNKERREK |
| 3300014024 | AKNYFSKVKILVDNKKITICNENTEKMEIEDFNEYDVRNRKFFNVLNKILNRENCTEEDLEVFENDLQK |
| SEQ ID NO: | RIGRKSIKNSLEENKAHFKKESINNNINYDKVKGNNKRSIFYEYYKNSLKHQEYINNIFEAFDKLYSNS |
| 4147 | HEAMNNLFSEITKDSKDRNIRKIREAYHEILNKNKTEFGEELYKKIQDNRNNFDKLLEIEPEIKELTKSQI |
| | FYKYYIDKVNLDETSIKHCFCHLVEIEVNQLLKNYVYSKRNINKEKLENIFEYCKLRNLVKNKLVNKLN |
| | NYIRNCGKYNAYISNNDVVVNSEKISEIRTKEAFLRSIIGVSSSAYFSLRNILNTDNTQDITNKVDKEVDK |
| | LYQENKKIELEEILKLFFGNYFDINNQQEIKVFLMNIDKIISSIRHEIIHFKMETNAQNIFDFNNVNLGNTA |
| | KNIFSNEINEEKIKFKIFKQLNSANVFDYLSNKDITEYMDKVVFSFTNRNVSFVPSFTKIYNRVQDLANS |
| | LEIKEWKIPDESEGKDAQIYLLKNIYYGKFLDKFLNEENIADIYVKLEKYNIGGSVKDRAALGMIEAAE |
| | KEGKLKPGGTIVEPTSGNTGIALALIGKAKGYRVIIIMPDSMSVERRSILAAYGAELILTEGAKGMKGAI |
| | AEAEKLASENGYFLPQQFENPANPAKHYETTAKEILDDFPQIDAFISGVGTAGTLSGVGKRLKEERPGV |
| | QVFAVEPATSAVLSGEQPGKHSQQGLGAGFIPGNYDANLVDGIIKITNEQAIEFATRASKENGLFVGISS |
| | GSAIAAAYEVAKKLGKGKKVLAVLPDGGEKYLSLEIFRKSL |
| UPLB01.1 | MLRRMCMKITKIDGISHKKYKEKGKLIKSNEIEKDITEERFNDIEAKTTELFQKTLDFYVKNYENSEDQ |
| SEQ ID NO: | NKERREKAKNYFSKVKILVDNKKITICNENTEKMEIEDFNEYDVRSGKYFNVLNKILNGENYTEEDLEV |
| 4148 | FENDLQKRIGRIKSIKNSLEENKAHFKKESINNNIIYDRVKGNNKKSLFYEYYRISSKHQEYVNNIFEAFD |
| | KLYSNSHEAMNNLFSEITKDSKERNIRKTREAYHEILNKNKTEFGEELYKKIQDNISNFDKLLEIEPEIKE |
| | LTKSQIFYKYYIDKVNLDETTIKHCFCHLVEIEVNQLLKNYVYSKRNINKEKLENIFEYCKLKNLVKNK |
| | LVNKLNNYIRNCGKYNAYISNNDVVVNSEKISEIRTKEAFLRSIIGVSSSAYFSLRNILNTDNTQDITNKV |
| | DKEVDKLYQENKKIELEERLKLFFGNYFDINNQQEIKVFLMNIDKIISSIRHEIIHFKMETNAQNIFEFNN |
| | VNLGNTAKNIFSNEINEEKIKFKIFKQLNSANVFDYLSNKDIREYMGKAVFSFTNRNVSFVPSFTKIYNR |
| | VQDLANSLEIKEWKIPDESEGKDAQI |
| QWBZ01.1 | MKISKIDDISHKKYKGKGKLIKSNEIEKDITEERFNDIEAKTKELFQKALDFYVKNYEKCEDQNKERRE |
| SEQ ID NO: | KAKNYFSKVKILVDNKKITICNENTEKMEIEDFNEYDVRSGKYFNVLNKILNGENYTEEDLEVFENDLQ |
| 4149 | KRIGRIKSIKNSLEENKAHFKKESINNNIIYDRVKGNNKKSLFYEYYRISSKHQEYVNNIFEAFDKLYSNS |
| | HEAMNNLFSEITKDSKDRNIRKIREAYHEILNKNKTEFGEELYKKIQDNRNNFDKLLEIEPEIKELTKSQI |
| | FYKYYIDKVNLDETSIKHCFCHLVEIEVNQLLKNYVYSKRNINKEKLENIFEYCKLRNLVKNKLVNKLN |
| | NYIRNCGKYNAYISNNDVVVNSEKISEIRTKEAFLRSIIGVSSSAYFSLRNILNSNNTQDITNDRILKQELD |
| | DIYQENNKKNKLEKNLKLFFGNYFDVMRESEIREFFTNIRDIIKRIRNKIIHFEMEANAQNIFDFNNINLG |
| | NTAKNIFNNEINEEKIKFKIFK |
| UPGN01.1 | MLRRMCMKITKIDGISHKKYKEKGKLIKNNDTAKDVTEERFYDIKTKTTELFQKTLDFYVKNYEQCEE |
| SEQ ID NO: | QNKERREKAKNYFSKVKLIIENRKITIFNENTEKIEIEGFNEYDVRDEKYFNVLNKILKEENCTEEDLEVF |
| 4150 | ENDLQKKLNQIQSIKNSLEENKAHFKKESVNNTADRVKGNNKKSLFYEYYRISSKHQEYANNIFEAFD |
| | KLYSNSHEAMNNLFSEITKDSKNRNIRKIREAYHEILNKNKTEFGEELYKKIQDNRNNFDKLLEIEPEIK |
| | ELTKSQIFYKYYIDKVNLDETSIKHCFCHLVEIEVNQLLKNYVYSKRNINKEKLENIFEYCKLRNLVKN |
| | KLVNKLNNYIRNCGKYNAYISNNDVVVNSEKISEIRTKEAFLRSIIGVSSSAYFSLRNILNSNNTQDITSD |
| | RILKQELDDIYQENNKKNKLEKNLKLFFGNYFDVMRELEIREFFANIRDIIKRIRNKIIHFEMEANAQNIF |
| | DFNNINLGNTAKNIFNNEINEEKMKFKIFKQLNSANVFDYLSNKDIREYMGKA |
| UPAU01.1_2 | LNTDNTQDITNKVKGEVEKLYQENKKVKLEERLKLFFGNNFDINNQQEIEDFLMNIDKIISNIRHEIIHFK |
| SEQ ID NO: | IEANAHNIFDFNNVTLGNKAKNIFNSEINEERIKFKIFKQLNSANVFDYLSDENITEYMGKVIFSFTNRNI |
| 4151 | PFVPSFRKIYNRVQDLANSLKIKEWKISDESEGKDAQIYLLKNIYYGEFLDDFLNEKNEKFIKIKDEIIEL |
| | NKNQNKITGFYKLEKFEKLKANTPTEYLEKLQSLHKINYNREKIEEDKDIYVDFVQKIFLKGFINYLQKS |
| | NSLKPLNLLNLKKDEVINSEKSSYDERKKYEQTDS |
| UPQF01.1 | MKVTKIDGISHKKFEDEGKLVRYTGNFNIKNEMKERLEKLKELKLSNYVKNPENVKNKDKNKEKETK |
| SEQ ID NO: | SRRENLKKYFSEIILRKKEEKYLLKKTRKFKNITEEINYDDIKKRENQQKIFDVLKELLEQRINENDKEEI |
| 4152 | LNFDSVKLKEVFGEDFIKKESKIKAIEESLEKNRADYRKDYVELENEKYEDVKGQNKRSLVFEYYKNP |
| | ENREKFKENIKYAFENLYTEENIKNLYSEIEEIFGKVHLKSKVRDFYQNRIIGESEFSEKDEEGISILYKQII |
| | NSVEKKEKFVEFLQKVKIKDLTKSQIFYKYFLENEELNDENIKYVFSYFVEIEVNKLLKENVYKTKKFN |
| | EGNKYRVKNIFNYDKLKNLVVYKLENKLNNYIRNCGKYNYHMENGCVATSDTNMKNRQTEAFLRSI |
| | LGVSSFGYFSLRNILGVNDNDFYEMEEELTEDERKNENFILKKAKEDITSKNIFEKVVDKSFEKKGIYQI |
| | KENLKMFYGNSFDKVDKDELKKFFVNMLEAITSVRHRIVHYNINTNSENIFDFSNIEVSKLLKNIFEKEI |
| | DTRELKLKIFRQLNSAGVFDYWESWKIKKYLENIEFKF |
| ODGY01.1 | MKVTKIDGLSHKKFEDEGKLVKFKNNKNINEIKERLKKLKELKLDNYIKNPENVKNKDKDAEKETKIR |
| SEQ ID NO: | RTNLKKYFSEIILRKEDEKYILKKTKKFKNINQQEIFDVLKEIKIKETEKEEIITFDSEKLKKVFGEDFVKK |
| 4153 | EAKIKAIEKSLKINKANYKKDSIKIGDDKYSNVKGEKKRSRIYEYYKKSENLKKFEENIREAFEKLYTEE |
| | NIKELYSKIEEVLKKTHLKSIVREFYQNEIIGESEFSKKNGDGISILYNQIKDSIKKEENFIEFIENIGNLKL |
| | KDLTKSQIFYKYFLENEELNDENIKFAFCYFVEIEVNNLLKENVYKIKRFNEGNKKRIKNIFEYGKLKKL |
| | IVYKLENKLNNYVRNCGKYNYHMENGDIATSDINMRNRQTEAFLRSIIGVSSFGYFSLRNILGVNDDDF |
| | YEIEEGLTEEERKNESNVLKKAKEDITSKSIFEKVVDKSFEKKGIHSIRKNVKMFYGDSFDKANEDELK |
| | QFFVNMLNAITSIRHRVVHYNMNTNSENIFNFSDIEVSRLLKSIFEKETDKRELKLKIFRQLNSAGVFDY |
| | WENWKIEKYLKNIEFKFVNKNIPFVPSFTKLYNRIDNLKAGNALKLGNHIIIPKRKEARDSQIYLLKNIY |
| | YGEFVEKFVNNNDNFEKIFREIIKINKNAGTNTKTKFYKLEKFETLKANTPTEYLEKLQSLHKINYDKEK |
| | VEEDKDTYVDFVQKIFLKGFINYLQKSNSLKPLNLLNLKKDEVINSEKSSYDEKLKQWENNGSKLSEM |
| | PKEIYEYIKKIQINKINYSNRMSIFYLLLKLIDHRELTNLRGNLEKYESMNKNEIYSEELNIVNLVSLDNN |
| | KVRANFNLESEDIGKFLKTETSIKNINQLNNFSGIFAD |
| UPKC01.1 | MKVTKIDGLSHKKFEDEGKLVKFRDNKNINEMKERLKKLKELKLDNYIKNPENVKNKDKDAEKETKI |
| SEQ ID NO: | RRTNLKKYFSEIILRKEDEKYILKKTKKFKNINQEIDYYDVKSKKNQQEIFDILKEILELKIKETEKEEIITF |
| 4154 | DSEKLKKVFGEDFVKKETKIKAIEKSLKINKANYKKDSIKIGDDKYSNVKGENKRSCIYEYYKKSENLK |
| | KFEENIREAFEKLYTEENIKELYSKIEEVLKKTHLKSIVREFYQNEIIGESEFSKKNGDGISILYNQIKDSIK |
| | KEENFIEFIENIGNLELKDLTKSQIFYKYFLENEELNDENIKFAFCYFVEIEVNNLLKENVYKIKRFNEGN |
| | KKRIKNIFEYGKLKKLIVYKLENKLNNYVRNCGKYNYHMENGDIATSDINMRNRQTEAFLRSIIGVSSF |
| | GYFSLRNILGVNDDDFYEIEEGLTEEERKNESNVLKKAKEDITSKSIFEKVVDKSFEKKGIHSIKENLKM |
| | FYGDSFDKANGDELKQFFVNMLNAITSIRHSVVHYDMNTNSENIFNFSDIEVSRLLKSIFEKETDKRELK |
| | LKIFRQLNSAGVFDYWENWKIKKYLENIKFEFVNKNVPFVPSFTKLYNRIDNLKGSNALNLGYINIPKR |
| | KEARDSQIYLLKNIYYGEFVEEFIKNNDNFEKIFREIIEINKNAGRNKQTNFYKLEKFEKLKAN |
| UPJV01.1 | MKVTKIDGLSHKKFEDEGKLVKFRNNKNINEIKERLKKLKELKLDNYIKNPENVKNKDKDAEKETKIR |
| SEQ ID NO: | RTNLKKYFSEIILRKEDEKYILKKTKKFKDINQEIDYYDVKSKKNQQEIFDVLKEILELKIKETEKEEIITF |
| 4155 | DSEKLKKVFGEDFVKKEAKIKAIEKSLKINKANYKKDSIKIGDDKYSNVKGENKRSRVYEYYKKSETH |
| | EKFRKNIIEAFEKLYTEENIKELYSKIEEVFKKTHLKSIVREFYQNEIIGESEFSKKDENGKSILYNQIEDSI |
| | KKDENFVEFLENIENLQLKELTKSQIFYKYFLENDLIDIIASDAHNLSTRKPYMKKAYDIIVDKYGKKRA |
| | ENLFYKTPARIMMERD |
| UPAZ01.1 | MKVTKIDGLSHKKFEDEGKLVKIEDASQKNETLERLENLKGIKLGNYIKNPDKTKNKDNKKRRKGLKE |
| SEQ ID NO: | YFSEITLRKENEKYVLLKGKKLKKINNDIKDTDIKAKDKKEEVFDILKEILKLNLLANDAEEKIQFDSIKL |
| 4156 | KNVFGKDFVKKELQIKSIEESLEKNKADYRKEFIETENHKYGNVKGKNKRSRIYEYYKKSENHKKFED |
| | NIREAFEKLYTEENIKELYSKIEQVLKKTHLKSIVREFYKNEIIGESEFSKKNGDRISILYNQIKDSIKKEE |
| | NFIEFIENIGNLELKDLTKSQIFYKNIKKVTGASFHYIILMYNCQLLFHIFWISFNFVV |
| IMG_ | MKVTKIDGLSHKKFEDEGKLVKIEDASQKNETLERLENLKGIKLGNYIKNTDKTKNKDNKKRRKGLK |
| 3300008454 | EYFSEITLRKENEKYVLLKGKKLKKINNDIKDTDIKAKDKKEEVFDILKEILKLNLLANDAEEKIQFDSIK |
| SEQ ID NO: | LKNVFGKDFVKKELQIKSIEESLEKNKADYRKEFIETENHKYGNVKGKNKRSHIYEYYKKSENHKKFE |
| 4157 | DNIREAFEKLYTEENIKELYSKIEQVLKKTHLKSIVWEFYKNEIIGESEFSKKNGDGISILYNQIKDSIKKE |
| | ENFIEFIENIGNLELKDLTKSQIFYKYFLENEELNDENIKFVFCYFVEIEVSDLLKGNVYKASKI |
| UPGO01.1 | MTIHKSKGLEFPVVIIAGMDKKRNIKSSSEMIRTSEKMGIGIDIIDDILKYKYPSIYKEIIGLEKTKEEKEEE |
| SEQ ID NO: | LRILYVAMTRAKEKLIMTAKVKSVEKLLQKLNESVKLNIYNNKLSSKCIMSIDTYLEIIMMSLTEAYNT |
| 4158 | QKVGKELEIKIDKNDFLVYSKSVENVIKINEKSDIKIGDIYIENIGNLELKDLTKSQIFYKYFLENEELNDE |
| | NIKNIFCYFVEIEVSDLLKGNVYKASKIYENKIKNIFEYGKLKNLIVYKLENKLNNYVRNCGKYNYHME |
| | NGDIATSDINMRNRQTEAFLRSMIGVSSFGYFSLRNILGVNDDDFYETEEDLTKAKKDITIKKIFEEVVD |
| | KSFEKKGIHNIKENLEMFYGDSFDKANEDELKQFFVNMLNAITSIRHRVVHYNMNTNSENIFNFSDIEV |
| | SRLLKNIFEKETDKRELKLKIFRQLNSAGVFDYWESWKIKKYLENIKFEFVNKNIPFVPSFTKLYNRIDD |
| | LKAGNALKLGNHIIIPKRKEARDSQIYLLKNIYYGKFVEEFIKNNDNFEKIFREIIEINKNAGTNKQTNFY |
| | KLEKFEKLKANTPTEYLEKLQSLHKINYNREKIEEDNI |
| UPQL01.1 | MKEGKLKKIIKIDWTIFYSKPRIQILGILIFLDIILLFSVTKEMEKGFSIYSVSTSIVLFILFILLNGLFIFYYK |
| SEQ ID NO: | NKFPNIEFYDDYFIFKKEKVYYENLKYFFFKDNRVFQMKKFSKILYKPDGGNWKKIDGSGYDYDLFSV |
| 4159 | FQKCFLEKNFLKAVENIENGGVEIFPFQNQGFVKNKFLFSSEEGLQELTQIFENSPKIQVSNKSVKFDNEI |
| | YDWENYNIEFEIGTITVSDLKKNTILEIETKNTVICQEILLKKLIENKLLNKLDTYVRNCGKYNYYLQVG |
| | EIATSDFIARNRQNEAFLRNIIGVSSVAYFSLRNILETENENDITGRMRGKTVKNNKGEEKYVSGEVDKI |
| | YNENKQNEVKENLKMFYSYDFNMDNKNEIEDFFANIDEAISSIRHGIVHFNLELEGKDIFAFKNIAPSEIS |
| | KKIFQNEINEKKLKLKIFRQLNSANVFN |
| UPUH01.1 | MKVTKVDGISHKKYIEEGKLVKSTSEENRTSERLSELLSIRLDTYIKNPDNASEEENRIRRENLKEFFSNK |
| SEQ ID NO: | VLYLKDGILYLKDRREKNQLQNKNYSEEDISEYDLKNKNSFSVLKKILLNEDINSEELEIFRKDVEAKL |
| 4160 | NKINSLKYSLEENKANYQKINENNIKKSLLPIFIDSYITDSTLTGGINPQIGEDYIKTISILNFPGFSVPGMI |
| | DRLNRTDIEYIWGSRYIMLEKITIKKILDKYYNKWWAARLSFKDMFIEFFSKNETTNPNQSAMNAAIEV |
| | RDEKTKLDEDRDIVGYYTTTVILKNKNRDVVERQAQEVRTLLSSLGFVVQIEDFYTLDCWLGVMPGN |
| | NYFNERRPFMNSKVLSHMLPINSVWAGNKWNKHLDTPPLLYCQTTGNTPFRLNLHYTDVGHTLIVGP |
| | TGSGKTLLAQTLAKILNVPFAIADATSLTEAGYVGEDVENIVLKLVQAADFDIEKAQRGIIYIDEIDKISR |
| | KSDNPSITRDVSGEGVQQALLKILEGTVANVPPTGGRKHPQQELIHIDTTNILFICGGAFVGLDKIVADRI |
| | GKKGIGFNSDVAKNVKEGESELIAKVMPQDLHKFGMIPELLGRIPVITSTRELVEEDLMSILTDPKNALT |
| | KQYKRMFELEGVDLEFTEDSLREIAKKALARGTGARGLRAICESTLQETMFDLPSDLDITKVVVTPESV |
| | GGDNAPEIIRGKKG |
| IMG_ | MKVTKRKGFNIKDVFHEKKEDKGVLSKIDDENDYMENKFKELASISLSTFIKDPVKSTKEENKKRREG |
| 3300000059 | LKEYFKNIEIYLKSEEVKINDKQDNKNTPSEGVEQTDLKESTKEIGKDVFNNIIKGEAHNLECFKKKLEE |
| SEQ ID NO: | HKKYLEKVKKSLNKNKSQYKVEQNQVSGTSKRNKFYDFYAKLNKLGEYKCRIEKAFDSLYSKNDILK |
| 4161 | IKENLTKKEEDNKKEGKNNKEEKFKKKEFFDSCKAILGSKINKDIQTDEGVTLKYIEGLKDHPLTQSRFF |
| | YKYXLSDEKNELTEENIKYCFPHFIEIEMHYLLRSLVKLNAKQRKEKAENIFKSHEIIKCYIKNKLKNKLI |
| | LYIQNSGKIKEYHSKYKGAIESSHLSDIRKGEGFIRNVIGATSSAYFSFRNIVNPKEKDDLLGKCMCCYP |
| | KETEKQKANIDSLDIYRVKQMLAIFYGEYFCGLKDDEVRCFLEVIKNSI |
| IMG_ | MKKILFLVALLPLTLVAQTVIVPNRYAFQKEDNQYQLNMLTKFLLEKQGFKVYMESEAPAELLQNPCD |
| 3300008727 | ALKADVKNESNMMTSKVQFLLTDCTNKAVFTSQIGKSREKEFKKSYQEALRNALSGTELATFKADYQ |
| SEQ ID NO: | APSVASKPSIPSATTAVPELTATAAPISEPLILFLYAKPTNWGYELFDKKTNELQFKLRKINTPDVFLAFD |
| 4162 | VEEQKYGILDLLEKIVTKADLKITKEEIKKYKNLQKELEKNDFYKIQEKIHRKYNQKPNLISRTENKKDF |
| | NDYKKAIENIQNYTQLKNKIEFNDLNLLQGLLFRILH |
| IMG_ | LQDCIKRAVKRTTEAARNINGGKTDEELILARAGKLSAIQKNERVQWFFAGLMADSTSEGRVQKDNY |
| 3300013000 | KHDADEAQKKAEYIEEIKQDVVALAFADYLKQFSFILDIKNILYADRNFPVEALKKTLREERKTAEEKT |
| SEQ ID NO: | KQSGEKADWICAKLYFLLHLIPVEEVSNLRQQIRKWEIVVDKPEVATAADEAENKEIANNGQPLEARQ |
| 4163 | QKALTEPIIQALDLYIFMHDAQYVGEEIGKVTADWAERFFEHGKGAMDRVFPAQDRQKESQAFRDLR |
| | EMRRFGNAVLHDIYAQNKISSKKIEEWRSQKAKVEGKKGLQTELQKLHENWVNNRKNPEWQRKGKD |
| | ESEGEKYKAYRETLAAVEAYRLLAGEVKLQDHLRLHRLLMAVLGRLVDFSGLFERDLYFALLALCHE |
| | KGVKDIKAVFKDSKGDETPFEETDENYGWNRFQNGQIFKAVDQLKEDYASIKNELVKFFGDIEKKGSS |
| | RNIRNRFAHLKMLTPPKEGEFSLHQGVHINLTQEVNKARQLMSYDRKLKNAVTKSIIELLEREGLKLS |
| | WQIQSGDAAEPAAKSGEGAAPASKKVSHNVRNPHIETKWIPHLGGKLLDKKDTDGKIVRDAKGNPVK |
| | EAITERHYGDTYLAMVELLFRG |
| IMG_ | LQDCIKRAVKRTTEAARNINGGKTDEELILARAGKLSAIQKNERVQWFFAGLMADSTSEGRVQKDNY |
| 3300013001 | KHDADEAQKKAEYIEEIKQDVVALAFADYLKQFSFILDIKNILYADRNFPVEALKKTLREERKTAEEKT |
| SEQ ID NO: | KQSGEKADWICAKLYFLLHLIPVEEVSNLRQQIRKWEIVVDKPEVATAADEAENKEIANNGQPLEARQ |
| 4164 | QKALTEPIIQALDLYIFMHDAQYVGEEIGKVTADWAERFFEHGKGAMDRVFPAQDRQKESQAFRDLR |
| | EMRRFGNAVLHDIYAQNKISSKKIEEWRSQKAKVEGKKGLQTELQKLHENWVNNRKNPEWQRKGKD |
| | ESEGEKYKAYRETLAAVEAYRLLAGEVKLQDHLRLHRLLMAVLGRLVDFSGLFERDLYFALLALCHE |
| | KGVKDIKAVFKDSKGDETPFEETDENYGWNRFQNGQIFKAVDQLKEDYASIKNELVKFFGDIEKKGSS |
| | RNIRNRFAHLKMLTPPKEGEFSLHQGVHINLTQEVNKARQLMSYDRKLKNAVTKSIIELLEREGLKLS |
| | WQIQSGDAAEPAAKSGEGAAPASKKVSHNVRNPHIETKWIPHLGGKLLDKKDTDGKIVRDAKGNPVK |
| | EAITERHYGDTYLAMVELLFRG |
| IMG_ | LQDCIKRAVKRTTEAARNINGGKTDEELILARAGKLSAIQKNERVQWFFAGLMADSTSEGRVQKDNY |
| 3300012998 | KHDADEAQKKAEYIEEIKQDVVALAFADYLKQFSFILDIKNILYADRNFPVEALKKTLREERKTAEEKT |
| SEQ ID NO: | KQSGEKADWICAKLYFLLHLIPVEEVSNLRQQIRKWEIVVDKPEVATAADEAENKEIANNGQPLEARQ |
| 4165 | QKALTEPIIQALDLYIFMHDAQYVGEEIGKVTADWAERFFEHGKGAMDRVFPAQDRQKESQAFRDLR |
| | EMRRFGNAVLHDIYVQNKISSKKIEEWRSQKAKVEGKKGLQTELQKLHENWVNNRKNPEWQRKGKD |
| | ESEGEKYKAYRETLAAVEAYRLLAGEVKLQDHLRLHRLLMAVLGRLVDFSGLFERDLYFALLALCHE |
| | KGVKDIKAVFKDSKGDETPFEETDENYGWNRFQNGQIFKAVDQLKEDYASIKNELVKFFGDIEKKGSS |
| | RNIRNRFAHLKMLTPPKEGEFSLHQGVHINLTQEVNKARQLMSYDRKLKNAVTKSIIELLEREGLKLS |
| | WQIQSGDAAEPAAKSGEGAAPASKKVSHNVRNPHIETKWIPHLGGKLLDKKDTDGKIVRDAKGNPVK |
| | EAITERHYGDTYLAMVELLFRG |
| IMG_ | LGHGPRTSPSAARRKPDEIPCRPGRGRATQWCTADPGPGTAWLPPAAPRPGRAGQAAPFSPRPDPFGPS |
| 3300031965 | RPHHSAPYIEDLKCDVVALAFAAWLKEADFDFLLALSADTPKPEIPLCDLDRIDLPAVNTRAADWQKA |
| SEQ ID NO: | LYFLVHLVPVDDIGRLLHQMRKWELLAKDSEPAGGIAMERIRQIQAALELYLYMHDAKFEGGAALAG |
| 4166 | IGEFKALFDSDDAFARIFPLQPGADDDRRVPRRGLREIVRFGHLPALLPVFGKHRIATAEVDEYLRLEHP |
| | QEDGKSEIARLQAHREALHEEWTEKKKDFAGDRLRTYVETLAAVVRHRHLAAHVTLTDHVRLHRLL |
| | MAVLGRLVDYSGLWERDLYFVTLALVHEAGCRPGEVFTDKGRKRLGQGRIVDALRDFQQTPDAGRIK |
| | DGLRRYFSAVWEKGNCSVRRRNNFAHFDMLKPANLPVDLTACVNDSRDLMAYDRKLRNAVSQSVRE |
| | LLHREGLDFEWTMDPAAPHRLGAATMESRGAPHLGGMRVPEKRVPQGRGRARRRQILENLHGDRFIA |
| | MAAALFGRCSPQRPESVVDWRPDAMDWSPPRGKNRNPGGKNRRGNGHGGGRKHGNAGRKPGRPV |
| IMG_ | LAGPEQIAKSRFWTSDWQAKIKRAEAFVRIWRHALALAGLTLKDLVDITDDILGGEGARKKALAALRA |
| 3300032892 | DPSKQAHFDQKRTVLFGEGVRKEEGKKPSLLDVVDRCDLASGLIDGAAKLRHAVFHFKGREYFLDEL |
| SEQ ID NO: | AELPKRFPANVGAAAQQLWQSDVTGRAARLNADLVAVHVPLFLTQEQAAQVFALLAADTIAEVPLPR |
| 4167 | FSRLLERARPWVEDKDAGVRLPEPANRRDLEDPARLCQYTLIKCIYERPFRAWLARQPAAAIAGWYDR |
| | AVARSSAAAKQENAKGDAVAERVITARAAALPKPAKDGDVVTFLFDLSRATASEMRVQRGYESDPD |
| | KARAQAEFIDRLLRDVVILALSAYLTKEKLGWVLDLKPGQIPAEPPLSSLDDVKAPEAAGEAEKWPAA |
| | LYLLLHLLPVEAVGQLLHQLFRWNTAATRETDLPEPEERRRQRLEAAMTLYLDMHDAKFEGGSPLQQ |
| | YEAFRGLFASGRGFERVFPRVSDQKAEQRIPKRGLREIMRFGRLALVKAICRDSTIDDGTVGAVMANE |
| | DSEGKDKSKIAALQERREELHEKWVKQKRLDKDDLRDYCATLNAIAQHRHAANFVYLVDHVRAHRA |
| | IMAVLGRLVDYAGLFERDLYFVTLALLHQNSLRPQEFFNTKGLEDVRNGEIISALHERKGDAPQAAGV |
| | EQKLARHFTKIWGPKNRIRGIRNDLMHLNMLQASPPTPRLTHWINEARELMAYDRKLKNAVSKSIIELL |
| | AREGLAARWTIRTSGGAHDLADGILSSRCAEHLGGMKLKLRGADRRDKGQPIAERLHSDAFVGMVAA |
| | AFDGKPVKADSILDSLSTVNWEASVHTKRHGDRGGPSRPHSPREKLRPGQRRRDREGRSGAKPDVRA |
| | K |
| IMG_ | VNRDDVAWINQHWPDDTTKSRYWTSDGQTEIKRHEIFVRIWRSALAHANRTLADWLDPDGKATDIAE |
| 3300007987 | GGNTFEQVLAATGQATVVQTANLFGSRAAALESHDAIQDLARLVWTVYTQLRHNSFHFKGVDGFKV |
| SEQ ID NO: | ALTPKLAEIAPGALQFTTNLLRGDFRDSEARLRAVLRAAQVEGFLDRGRLAEIWAALHPAGEAGLGPA |
| 4168 | LPRFNRIVQRVDGTGAIAELPCAVNQFDMANPAIHCRYVVTKLLYERGFRAWVAMATTDQINAWIKS |
| | AEDRTQKAKDEIHGHPEDKARMVGALRLRDGQGIMDFLSELTALTATEFRVQAGYEPNRAAAQEQAE |
| | YLFHLQCDVLALAFKEYIRAARLGWLSDTLKPDRVARGVLSDVDKLADPKATPDFEDWQAGIYAVLH |
| | LVPVDEVGRLLHQLRRWANGQPADETSVKIERLLELYLDMHDDKFEGGVPLHDHPDLQALFETPDLL |
| | AQVLPQVGATEDERRRVPLRGLREMLRFGNLRILKGVFAKAPITTAGVAKLAEYEKSRADGLGGIDHA |
| | QQIRQKRHETLAKLRMLGPDSYGDVRDYLFVTKRIIRHRRLANHVRLVNHVRVNQILMSVLGRFADC |
| | AGLFERDLYFTLLALIHELGQKPEAVFDAKALGKFEDGQILTALDRNKTPLPASIACELQRHFGLDAKG |
| | AGPNRKRRNDFAHFNLLRSKTPINLTAAMNDARALLSHDRKLKNAVSASIVTLLEREQIVAHWNMGT |
| | DHQLADAVIGTRRIVHLKSAELAENLRDKRFLTLIAQLFNGRVNNLPDDIAALDRPGIEALAARVTGGG |
| | GR |
| IMG_ | VNRDDVAWINQHWPDDTTKSRYWTSDGQTEIKRHEIFVRIWRSALAHANRTLADWLDPDGKATDIAE |
| 3300009004 | GGNTFEQVLAATGQATVVQTANLFGSRAAALESHEAIQDLARLVWTVYTQLRHNSFHFKGVDGFKV |
| SEQ ID NO: | ALTPKLAEIAPGALQFTTNLLRGDFRDSEARLRAVLRAAQVEGFLDRGRLAEIWAALHPAGEAGLGPA |
| 4169 | LPRFNRIVQRVDGTGAIAELPCAVNQFDMANPAIHCRYVVTKLLYERGFRAWVAMATTDQINAWIKS |
| | AEDRTQKAKDEIHGHPEDKARMVGALRLRDGQGIMDFLSELTALTATEFRVQAGYEPNRAAAQEQAE |
| | YLFHLQCDVLALAFKEYIRAARLGWLSDTLKPDRVARGVLSDVDKLADPKATPDFEDWQAGIYAVLH |
| | LVPVDEVGRLLHQLRRWANGQPADETSVKIERLLELYLDMHDDKFEGGVPLHDHPDLQALFETPDLL |
| | AQVLPQVGATEDERRRVPLRGLREMLRFGNLRILKGVFAKAPITTAGVAKLAEYKKSRADGLGGIDHA |
| | QQIRQKRHETLAKLRMLGPDSYGDVRDYLFVTKRIIRHRRLANHVRLVNHVRVNQILMSVLGRFADC |
| | AGLFERDLYFTLLALIHELGQKPEAVFDAKALGKFEDGQILTALDRNKTPLPASIACELQRHFGLDAKG |
| | AGPNRKRRNDFAHFNLLRSKTPINLTAAMNDARALLSHDRKLKNAVSASIVTLLEREQIVAHWNMGT |
| | DHQLADAVIGTRPIVHLKSAELAENLRDKRFLTLIAQLFNGRVNNLPDDIAALDRPGIEALAARVTGGG |
| | GR |
| IMG_ | VAWINQHWPDDTTKSRYWTSDGQTEIKRHEIFVRIWRSALAHANRTLADWLDPDGKATDIAEGGNTF |
| 3300025017 | EQVLAATGQATVVQTANLFGSRAAALESHEAIQDLARLVWTVYTQLRHNSFHFKGVDGFKVALTPKL |
| SEQ ID NO: | AEIAPGALQFTTNLLRGDFRDSEARLRAVLRAAQVEGFLDRGRLAEIWAALHPAGEAGLGPALPRFNRI |
| 4170 | VQRVDGTGAIAELPCAVNQFDMANPAIHCRYVVTKLLYERGFRAWVAMATTDQINAWIKSAEDRTQ |
| | KAKDEIHGHPEDKARMVGALRLRDGQGIMDFLSELTALTATEFRVQAGYEPNRAAAQEQAEYLFHLQ |
| | CDVLALAFKEYIRAARLGWLSDTLKPDRVARGVLSDVDKLADPKATPDFEDWQAGIYAVLHLVPVDE |
| | VGRLLHQLRRWANGQPADETSVKIERLLELYLDMHDDKFEGGVPLHDHPDLQALFETPDLLAQVLPQ |
| | VGATEDERRRVPLRGLREMLRFGNLRILKGVFAKAPITTAGVAKLAEYEKSRADGLGGIDHAQQIRQK |
| | RHETLAKLRMLGPDSYGDVRDYLFVTKRIIRHRRLANHVRLVNHVRVNQILMSVLGRFADCAGLFER |
| | DLYFTLLALIHELGQKPEAVFDAKALGKFEDGQILTALDRNKTPLPASIACELQRHFGLDAKGAGPNRK |
| | RRNDFAHFNLLRSKTPINLTAAMNDARALLSHDRKLKNAVSASIVTLLEREQIVAHWNMGTDHQLAD |
| | AVIGTRPIVHLKSAELAENLRDKRFLTLIAQLFNGRVNNLPDDIAALDRPGIEALAARVTGGGGR |
| IMG_ | VAWINQHWPDDTTKSRYWTSDGQTEIKRHEIFVRIWRSALAHANRTLADWLDPDGKATDIAEGGNTF |
| 3300025835 | EQVLAATGQATVVQTANLFGSRAAALESHEAIQDLARLVWTVYTQLRHNSFHFKGVDGFKVALTPKL |
| SEQ ID NO: | AEIAPGALQFTTNLLRGDFRDSEARLRAVLRAAQVEGFLDRGRLAEIWAALHPAGEAGLGPALPRFNRI |
| 4171 | VQRVDGTGAIAELPCAVNQFDMANPAIHCRYVVTKLLYERGFRAWVAMATTDQINAWIKSAEDRTQ |
| | KAKDEIHGHPEDKARMVGALRLRDGQGIMDFLSELTALTATEFRVQAGYEPNRAAAQEQAEYLFHLQ |
| | CDVLALAFKEYIRAARLGWLSDTLKPDRVARGVLSDVDKLADPKATPDFEDWQAGIYAVLHLVPVDE |
| | VGRLLHQLRRWANGQPADETSVKIERLLELYLDMHDDKFEGGVPLHDHPDLQALFETPDLLAQVLPQ |
| | VGATEDERRRVPLRGLREMLRFGNLRILKGVFAKAPITTAGVAKLAEYEKSRADGLGGIDHAQQIRQK |
| | RHETLAKLRMLGPDSYGDVRDYLFVTKRIIRHRRLANHVRLVNHVRVNQILMSVLGRFADCAGLFER |
| | DLYFTLLALIHELGQKPEAVFDAKALGKFEDGQILTALDRNKTPLPASIACELQRHFGLDAKGAGPNRK |
| | RRNDFAHFNLLRSKTPINLTAAMNDARALLSHDRKLKNAVSASIVTLLEREQIVAHWNMGTDHQLAD |
| | AVIGTRPIVHLKSAELAENLRDKRFLTLIAQLFNGRVNNLPDDIAALDRPGIEALAARVTGGGGR |
| IMG_ | VAWINQHWPDDTTKSRYWTSDGQTEIKRHEIFVRIWRSALAHANRTLADWLDPDGKATDIAEGGNTF |
| 3300025825 | EQVLAATGQATVVQTANLFGSRAAALESHEAIQDLARLVWTVYTQLRHNSFHFKGVDGFKVALTPKL |
| SEQ ID NO: | AEIAPGALQFTTNLLRGDFRDSEARLRAVLRAAQVEGFLDRGRLAEIWAALHPAGEAGLGPALPRFNRI |
| 4172 | VQRVDGTGAIAELPCAVNQFDMANPAIHCRYVVTKLLYERGFRAWVAMATTDQINAWIKSAEDRTQ |
| | KAKDEIHGHPEDKARMVGALRLRDGQGIMDFLSELTALTATEFRVQAGYEPNRAAAQEQAEYLFHLQ |
| | CDVLALAFKEYIRAARLGWLSDTLKPDRVARGVLSDVDKLADPKATPDFEDWQAGIYAVLHLVPVDE |
| | VGRLLHQLRRWANGQPADETSVKIERLLELYLDMHDDKFEGGVPLHDHPDLQALFETPDLLAQVLPQ |
| | VGATEDERRRVPLRGLREMLRFGNLRILKGVFAKAPITTAGVAKLAEYEKSRADGLGGIDHAQQIRQK |
| | RHETLAKLRMLGPDSYGDVRDYLFVTKRIIRHRRLANHVRLVNHVRVNQILMSVLGRFADCAGLFER |
| | DLYFTLLALIHELGQKPEAVFDAKALGKFEDGQILTALDRNKTPLPASIACELQRHFGLDAKGAGPNRK |
| | RRNDFAHFNLLRSKTPINLTAAMNDARALLSHDRKLKNAVSASIVTLLEREQIVAHWNMGTDHQLAD |
| | AVIGTRPIVHLKSAELAENLRDKRFLTLIAQLFNGRVNNLPDDIAALDRPGIEALAARVTGGGGR |
| IMG_ | MATAVSIGKLIHYQGGIEAIGNKEDLVNSKFLTDAGLTEIKQNESFVRQWLELIAIANTTLSQLVDPDGK |
| 3300008225 | HEDIFAATSFDDALKGLNNISDFDSKFKLLFGENHRLYDDDTERQKLLRIIYDTISALRNASFHFKNIQGF |
| SEQ ID NO: | NKALEDNLSTKGRRQKGVVDKIIEYTKVHQQKQHELLIADLKAANVEDYLSQLQLDYLFEVVCKGER |
| 4173 | ELLDMPKFSKLLLRAGEIGEKIISPVNATAMEHPAYQCCFISLKMLYDQDFVNWLKKQSNKNIASWMD |
| | SAKTRATNAAKKIFRNGTNISSKMARLRNIVEDETLVEYFSFITAETANEFQVQQQKNRYQSNSASAKE |
| | QSNFVEQFKQDFLIYAFKGYIGDIKFGKLDQGEKLLKCNAKGSLLPENNRSDTGAEDIERPWLYLVLHL |
| | VPIEVVNRITLQIKKHSVLTNSTLDDTRAAYTDIHQAFSLYLGVHGAILGLQSLSNEPELQVFFEKESDF |
| | NNLFTDNGEGLVPVKGLRDILRFGNLEQLKKMFSDKKVASDDITNLKEYLEATGGKSKIARAQEKRIE |
| | LHKTLTELPKRLTKPRVKQTGIDTYEKNNNISISGSISEYRKDLKCIVSYRELKNKIYLYNVKQAHQLIM |
| | ATQARLLGYSQIRERDLYFVLLTQLMLRGVTLEKEKKKKDKDKLNALDTDTVLYEEEKKKLEKQIKPE |
| | RSLKDLVEKGLIFLALDQLSSSDKDLKAIHSEIEDMFVGVSPDSDNRNLRNRLAHFKDLGNKNLINITSQ |
| | INEVRKMMSYDRKLKNAVSKSMIDLFERYNLILSFKVQSHKLQLKNLKSKQITHLNNKGITENLLSDD |
| | YVSVIKRLLLTEQNPE |
| IMG_ | MRTKRKQYKIKTKNNRKIDDILSDKSNLRAIFNDLRSNTELQKHFKEKLAFCYPIFIKVKKEKMFDDIEK |
| 5330000407 | LIKLVEEARESVAYLRHRCFHYKDVTITEMLKALNNNTETDKTEDIDYSVAAEYFLRDINNLYDAFRE |
| SEQ ID NO: | QIRSSGIADYYPADIISGCFKKCGLQFVLYSPQNSLMPSFKNIYKRGSNLYKAYQEEKEQKDKEYKRHN |
| 4174 | TNIVQEESKELSWYIEVSDTEQGKTAYRNLLQLIYYHAFLPEVRENESLITVYFAKTKEWNRKVAETKA |
| | KKKNAGKTYKDKPIRAYRYEAIPDYVGERLDDYFKILQREQMAKAKDVNEGNAENNNYIQFIRDVVV |
| | WAFGAYLEERLEKYKKDLQSSHSQKDKKDVNDALKELFPDDKDKRQFFMKCKFTDVLINDVGENNQI |
| | TEMEDLETSKEQQNREIKRKDLLCFYLFLRLLDEREISGLKHQFVRYRCSLKERRLPDNRKDVDEEIVL |
| | LEELEELMELVSYTMPSVPELSGKAESGLDLVISKYFKDFFEKSALKNQDIMKLYYQSDNKTPVFRKY |
| | MALLMRSAPLQLYKDMFRNYYIITEKECQEYIKTSQDIDAFQCKLNELHKELEHVRLKTVEDKKGKIF |
| | YYLAGSDAERVKEYEDTLSKVVRYKRLQHKLTFESLYTIFKIHVDIAARMVGYTEDWERDMLFLFKSL |
| | EYNEKLNEGVVEKIFNNKDEKGHIVKKLKDNLNSEDKEKIGILCWHKEITDKNFVEIIWIRNPIAHLNHF |
| | MQTVKNPKRSLEKMINALCVLLSYDRKRQNSVTKTINDLLLNEYHVKIKWKRWVDKNCNIYPELFMR |
| | VKNHRFTHEIITTVHFRG |
| IMG_ | MMPSFKNVFIRGCNITKGNFNLKECEWFKDKDTYNKDAYLAYKNLLQLIYYHSFLPSVSSDETIITKYI |
| 3300011885 | NKTKAWNQKIAIAKQKGKINKYQYKYNDMPNYQIGIKLSDYLSNLQRLQSIRENDDNIAEKGNYYTDF |
| SEQ ID NO: | VKDVFVFAFNGYLQSKIPNLCGTVKSPCKHNSKTILDDLFVDANLSLKMKTGHNKLSEFAGMYLFLKL |
| 4175 | LDQRELNKLLHQFIRYRTSTNKINEDLSKVEELIALVQFTLPPPTTDENYNENLEDYFSKFIDGNYMTDY |
| | VDLYSQEDKKTPILQRSISLIGRSGAMALYTDIFTQQVKSYTVTKSDYDKYYEYNFGHSSELSVIEKKQ |
| | NELQTLHKDIVTAKKDADIKEKVSKYETLVKEVQEYNQCRQKVTFETLYKVHQIHIDILGRFASFAED |
| | WERDMFFMLAALKRLGKTSLDVNKVFEEGGVVGKLSDALKTSKTLFCNLCWADDSVNERDIKFKIRV |
| | RNILAHLNHMTQYNEKGNQPSIIDIINKLRILLAYDLKRQNAVTKSIQDLLLKDYKIKLVLEPVKTKEEL |
| | KIFKIKSLDSDYIVHLKNIDSANSKKGIAIKANNNFMIELIEKLLVFKY |
| IMG_ | MRVTKVKVKDAGKDKMVLIHRKTTGAQLVYSGQPVSNETNTILPDKKRDSFDLSILNKTIIKFETVRK |
| 3300028769_2 | QKLNIDQYKTLEKIIKYPKQELPTQIKAEEILPFLNHKFQEPVKYWKEGKEEKFNLTLLIVEAVKAQDKR |
| SEQ ID NO: | ILQPYHEWKEWYIKTKSDLLKKSIENNRIDLSDNLSKRKKALQAWETDFTTTGSIDLSHYHKVYMTDV |
| 4176 | LCKMLQEVKPLTDERGKINTNAYHRELKKALQTHQPAIFGTREAPNETNRLNNQLSIYHLEVVKYMEH |
| | YFPIKTSKRRNTADDIVHYLKAQTLKTTIEKQLVNAIRANIIQQGKTNHHELKADTSSSDLTKIKTNEAF |
| | VLNLIGACAFAANNIRNMVDNEQTLDVLGKREFIDSLTGTRISSQLYSFFFGESLSTSKAEKETQLWGN |
| | TWSGTTNTE |
| IMG_ | MRVTKVKVKDAGKDKMVLIHRKTTGAQLVYSGQPVSNETNTILPDKKRDSFDLSILNKTIIKFETVRK |
| 3300028864_2 | QKLNIDQYKTLEKIIKYPKQELPTQIKAEEILPFLNHKFQEPVKYWKEGKEEKFNLTLLIVEAVKAQDKR |
| SEQ ID NO: | ILQPYHEWKEWYIKTKSDLLKKSIENNRIDLSDNLSKRKKALQAWETDFTTTGSIDLSHYHKVYMTDV |
| 4177 | LCKMLQEVKPLTDERGKINTNAYHRELKKALQTHQPAIFGTREAPNETNRLNNQLSIYHLEVVKYMEH |
| | YFPIKTSKRRNTADDIVHYLKAQTLKTTIEKQLVNAIRANIIQQGKTNHHELKADTSSSDLTKIKTNEAF |
| | VLNLIGACAFAANNIRNMVDNEQTLDVLGKREFIDSLTGTRISSQLYSFFFGESLSTSKAEKETQLWGN |
| | TWSGTTNTE |
| IMG_ | MRVTKVKVKDAGKDKMVLIHRKTTGAQLVYSGQPVSNETNTILPDKKRDSFDLSILNKTIIKFETVRK |
| 3300030002 | QKLNIDQYKTLEKIIKYPKQELPTQIKAEEILPFLNHKFQEPVKYWKEGKEEKFNLTLLIVEAVKAQDKR |
| SEQ ID NO: | ILQPYHEWKEWYIKTKSDLLKKSIENNRIDLSDNLSKRKKALQAWETDFTTTGSIDLSHYHKVYMTDV |
| 4178 | LCKMLQEVKPLTDERGKINTNAYHRELKKALQTHQPAIFGTREAPNETNRLNNQLSIYHLEVVKYMEH |
| | YFPIKTSKRRNTADDIVHYLKAQTLKTTIEKQLVNAIRANIIQQGKTNHHELKADTSSSDLTKIKTNEAF |
| | VLNLIGACAFAANNIRNMVDNEQTLDVLGKREFIDSLTGTRISSQLYSFFFGESLSTSKAEKETQLWGN |
| | TWSGTTNTE |
| IMG_ | MRVTKVKVKDAGKDKMVLIHRKTTGAQLVYSGQPVSNETNTILPDKKRDSFDLSILNKTIIKFETVRK |
| 3300031722 2 | QKLNIDQYKTLEKIIKYPKQELPTQIKAEEILPFLNHKFQEPVKYWKEGKEEKFNLTLLIVEAVKAQDKR |
| SEQ ID NO: | ILQPYHEWKEWYIKTKSDLLKKSIENNRIDLSDNLSKRKKALQAWETDFTTTGSIDLSHYHKVYMTDV |
| 4179 | LCKMLQEVKPLTDERGKINTNAYHRELKKALQTHQPAIFGTREAPNETNRLNNQLSIYHLEVVKYMEH |
| | YFPIKTSKRRNTADDIVHYLKAQTLKTTIEKQLVNAIRANIIQQGKTNHHELKADTSSSDLTKIKTNEAF |
| | VLNLIGACAFAANNIRNMVDNEQTLDVLGKREFIDSLTGTRISSQLYSFFFGESLSTSKAEKETQLWGN |
| | TWSGTTNTE |
| UOPF01.1 | MKVSKVKVKVGAGRSSERMVFMRRTSKIGSLVYEDEQRNGKPETDDKTTSILPDKKRDSFILSIVNKTI |
| SEQ ID NO: | PKKEIVKKNLGKGFVNEYYNAIAGIIDSFLEKKIVDRKHYIIVNKLTEEEIKQYLNHRFQEANYKYVRD |
| 4180 | KEEVNFNLPKLLKESAKSNSTAPLQPYKEWAEWHIETKSVRLIRSIQNNRLVIDTQEEAENMSPRKRAL |
| | LKWENEFLLSHKLDLQDVEKTYLIDDLIHALHEVTYTTNDKGFINGNEYHRFLKKALQSHQQNIFGSRE |
| | TPNKVNRENAELYSYNMEVVKYLEHYFPIKKTNRRNTLDTKDYYLNGINIKDRVRKQLENAVRNNLV |
| | RQGKYTLHTLTTDTANSDNLSKIKADEGFALTMLNQCAFAANNVRNIIDPTQVEDILLDRPFNESLEKF |
| | NSAQMLHLSSFFDVKEFNEPLRAIRDAVAKIRHNIIHYKVNALNVIFKIETFGSTEKQYKDTIFGSLLQA |
| | DMMNVSESLAKQLMTGNVLEYYPMLELKSFFSKNSISLYRSVIPFAPGFKRVMKKGENYQNANNKDD |
| | KSKYYNLKIESFLPQESFTKEAYDARYFLLKLIYNNIFLPKFTESTDWFKSTVNGVIALNREENVRKGKK |
| | HKIAFAEIRLMDSRDTIGTYAA |
| JMBX01.1 | LSAESSEKLFGKRAEGYDINRADNQLYVYNTEVVKYMEHYFPVKSSKRRNSTAEIKYYLQTDTIKCCL |
| SEQ ID NO: | HHQIINAVRGLALREGKFNLHGFDDKLIPNERNVSSSILNELKTSEGFVLNMLGGCAFAANCLRNIVDA |
| 4181 | TQRSDLLGFRCFEVSLKKGKSNSDLFALFFGFGREDMDDDSEWEKHLYAARYSVSEIRNRVAHYHKS |
| | AIENIYNITDFKYRENSMCSYTDTKFTTALQNEIYNTPKALSLQLMTGKVLEYYPKEKLVSFFQKYKFS |
| | LYRSVVPFAPGFKNIMRTGVNYQNATQNSLFL |
| IMG_ | MRVSKVKVDKEMVLMHRNNKEGALIIGNSTDNKTNYILPKKKKENFYKSIINKTLVKDIKFIDEYKKT |
| 3300014204 | RTKPRDRDIELTLTNLIEKNNAHPLKNKDIETINKNLRGKFNKYLSYNGNEPFNLAELIYEYSTKNDIKIP |
| SEQ ID NO: | QPYKDWVEWYIETKSKFLIKSIENNRIVIENGEEKLSKRKKVLIGFEEKLKEKGEIDLSDVANKFNITSLV |
| 4182 | KEISPKVEEYIEKDKKRTYKDKNNNKLERELNFAIKDTLQEHQKGIFGTRENPKERDNDKLSIYNLEVV |
| | KYIEHYFPIKKSQRTYNIGSIKHHISEETIKSTIQHQIENAVRLNMIHLGKSIHHEYKNSISSTDLSNTKRQ |
| | EAFVLNMIGACAFATNNIRNIIDSEQGEDILVRKAFTDSLNKGKVDYNLLKLFLGKGSNSNEETLWALR |
| | GSIRGIRNNVIHYKKDAIEKIFKIEVFENPINGNDQNETPYSKSIFGKYLQEDISKLSGLFANQLMTGGVL |
| | SYYSIDDLKAILDKIEFNLCRSSIPFTPSFKKVFKGGRDYQEKKPSLNLNNYITKEKNHETEEEYQARYFL |
| | LKLLYNNIFIPSFEGNYFREAAKYVLEENKNNAL |
| GCA_900114365. | MKVSKVKVSVGNNEKQMMTMFRNSNKGALVYWDDKSRDDQTERIIPGQKMENFALSILNQTLVKKG |
| 1_IMGtaxon_ | VFLSMLRMGTSGKVASKHANGTEMRVTHKEKEKAGKAYESIRALLAFVLSSDFGSREFKKNVPKEIER |
| 2651870357_ | SLLDCMITKKFREEIYLMDEKTGEKRRLTDLILEALSSGDVLILTPYVKWRDDFVALKSSFLRRSIHNNR |
| annotated_ | ITVANGGSKRMSVLEAWSEALISPEKDQTEKNKVQGFSKINAISEVPTRYNIDLLIKNLNKVEMGEFKD |
| assembly_ | NGTLKRGHEFHKRLKVCLQTHQKTIFGTRDNPNLTNRGDNELYCYNLEVVKYLNHFFPINVPSAKRLT |
| genomic | KDRILYYLNEKTMKRTIEAQLHNALRANLIRNGKLRWHDLLGRDDITNKDLITLKMDEGFLLSIIDACA |
| SEQ ID NO: | FAGNNVRNIIDRYQTGDIIYKDILKKSIEKGVSDGPLFGLFFNIEDSQPILTKDLWALRGAVQKIRNDIFH |
| 4183 | YMFNLPNNDGGMHDDRSATKVKTILNVTEFEYDGDNKTDKSSR |
| GCA_000525995. | LSCRLSSRSNPSIDATNPDWAKLFETLKPYTDWVESYIHFKQTTIQKSIEQNKIQSAHSPRKLVLHKYAT |
| 1_PRIP_ | AFLEGRVMGYESLAAKYQLADLAESFKVVDLNKNKNANYEIKKILQQHQRNILGELKTDPELNQYGIE |
| MIRA_assembly_ | VKKYIERYFPIKSKPKRNKHSRADFLKKELIESTVKQQFKNAVYHYVLEQGKLEAYNLTSPKTKDLQNI |
| genomic | RAGEAFSFKFINACAFASNNLKTILNPECEEDILGKNCFIQNLPDSTARPNVVQKMIPFFSDEIQNVNFDE |
| SEQ ID NO: | AIWAIRGSIQKIRNEVYHCKKHAWEKNTQNKRL |
| 4184 | |
| UPBG01.1 | MENNSEKKKYLKTLVGDNVYLSPISLDDVEEYTEMVNNIEVSVGLGCVVYTNIMDFESEKELLNSIKK |
| SEQ ID NO: | EKIFGVRLLENDELLGNVGFKSIGEIHRTAEMGIMLGNPKYQRKGYGMEAINLLLDYGFSFLNLRNISL |
| 4185 | NVFEYNEVAYNLYKKIDISKVTKNDKNIFQVSSLEGKLNVKIPYPVVTENKKQKSYNEETVKFLDEFIK |
| | AEVKAGLPSAQIAVTKDGNLELLSSYGYVNNYKQDGTELKDKVKVTDNTVYDLASNTKMYATNYAI |
| | MKLVSEKKLNLDDYVHKFYPEFKGNGKEKIQISDLLKHQAGFPPDPQYFNDKYDKDDGIPNGKNDLY |
| | AIGKEKVKNAIMKTPLAYEPKTSTKYSDVDYMLLGLIIEKVTSQDLDTYMKENFYNKLNLKRTMFNPL |
| | KNGVSKNETAATELNGNTRDNTIDFINARKYTIQGEVHDEKAYYSMQGVSGHAGLFSNAYEVAKLAQ |
| | VIINEGGYDNVKFFDKTTLDNFIKPKDINASYGLGWRRQGDFIYRWAFSGLASRETVGHTGWTGTLTVI |
| | EPSQNLVIVLLTNAKNSRVIDPSKKPNDFYGNHYYTTNYGVISSIIIDAFSNMNSKKDTNLRMNSILEDM |
| | IKGKFNLIKTDSDYKNSADIRDTVELINLLNLDNNRVTEDFELEADEIGKFLDFNGDKVKDRKELKKFD |
| | TKKIYFDGENIIKHRAFYNIKKYGMLNLLEKIADKAKYKISIEELRKVRT |
| UPGJ01.1 | MLGYYIAILWGVILFIIFPCYPLNKWVLHNKWNHSDWATFLGGFLGAFITLFGVWWQVTKTQKQKEK |
| SEQ ID NO: | DEMKNHLLGLKYNLEKNIKKFDYLYKNIIIFSYTLRSFYDRIDKGFFEEIDSNGVFIDTKIFNLNFTNDIL |
| 4186 | DLKNAIIDAKIAENDEAYIKNYIFESNEEKLKKRLFCEELIDKEDIRKIFEDKNFKFKNFIKKTENENFTIN |
| | FDNLFNLECNSELNVKKVIGQNSQRLNLFIKNTIDEYKSKIKTSFSSEFLEKYKGIIDNLIENENKFEKIYY |
| | PEEHKNELYIYKKNLFLNIGNPNFDKIYGLISNDIKEADAKFLFDSDGEDIRNNKISEIDAILKNLNDKLN |
| | GYSKEYKEKYIKKLKENNDFFKKNIQNENYNSFEEFKEDYNKVSEYKRIRDLVEFNYLNKIESYLIDIN |
| | WKLAIQMARFERDMHYIVNGLDYLYIIRLEKNRNQDRSSPYPKYKNGVLDYTKSYYNFKDYQEFMDI |
| | CSKFGIDLSENSEINKPENESIRNYISHFYIVRNPFVDYSIAEQIDRVSNLLSYSTRYNNSTYASVFEVFKK |
| | DVNLDYDKLKKKFKLIGNNDILKRLMKPKKVSVLELESYNSNYVKNLIIKLLTKIENTNDTL |
| IMG_ | MEMRRLEWEIYLDIKDGMKFLKIKRKVKVKRNYDGNKYILNINENNNKEKIDNNKFIRKYINYKKND |
| 3300008155 | NILKEFTRKFHAGNILFKLKGKEGIIRIENNDDFLETEEVVLYIEAYGKSEKLKALGITKKKIIDEAIRQGI |
| SEQ ID NO: | TKDDKKIEIKRQENEEEIEIDIRDEYTNKTLNDCSIILRIIENDELETKKSIYEIFKNINMSLYKIIEKIIENET |
| 4187 | EKVFENRYYEEHLREKLLKDDKIDVILTNFMEIREKIESNLEIMGFVKFYLNVGGDKKKSENKKILVEKI |
| | LNINVDLTVEDIADFVIKELEFWNITKRIEKVKKVNNEFLEKRRNRTYIKSYVLLDKHEKFKIERENKKD |
| | KIVKFFVENIKNNSIKEKIEKILAEFKIDELIICKLEKELKKGNCDTEIFGIFKKHYKVNFDSKKFSKKSDEE |
| | KELYKIIYRYLKGRIEKILISEEKVRLKKMKKIEIEKILNKSILSKKVLKRVKQYTLEHVMYLGKLVHNDI |
| | DMTTVNTNDFSMLHAKEELDLELIT |
| IMG_ | LFLWIIEEIIYEIIKLYKDLKEEIIMGNLFGYKRWYEVRDKEDYKIKRKVKVKRNYDGNKYILNINENNN |
| 3300014038 | KEKIDDNKFIREFVNYKKNDNVLREFKRKFHAGNILFKLKGKERIKRIENDDDFLETEEVVLYIEVYGK |
| SEQ ID NO: | SEKLKALGITKKKIIDEAIRQGITKDDKKIEIKRQKIEINIRDKYTNKTVDDCSVILRIIENDELETKKSIYEI |
| 4188 | FKNINMNLYKIIEKIIVNKTEKVFENRYYEEHLREKLLKDDKTEVILTNFMEIREKIKSNLEIMGFVKFYL |
| | NVGGDKKKSENKKIFVEKILNINVDLTVEDIVDFIVKELKFWNITKRIEKVKEFNNKFLENKRNRTYIKS |
| | YVLLDKHEKFKIERENKKDKIVKFFVENIKNNSIKEKIEKILAEFKIDELTKKLEKELKKGNCDTEIFGIF |
| | KKHYKVNFDSKKFSNKSDEEKELYKIIYRYLKGRIEKILINGEKVRLKKMEKIEIEKILNESILSEKILKRI |
| | KQYTLEHIMYLGKLVHNKINMATVNTNDFFRLHAKEELDLELITFFASTNMELNKIFSRENINNDENID |
| | FFGGDREKNYVIDKKNLNSKIKIIRDLDFIDNKNNITNDFINKFTKIGTNERNRILHASGKKRDSQGTQD |
| | DYNKVINIIQNLKISDEEVSKALNLDVVFKDKKNIITEINDIKISEENSNDIKYLPSFSKVLPEILNLYRNNP |
| | KNKPFDTIETEKIVLNALIYVNKELYKKLILEDDLKKNRSENIFLQELKKTLGNIDETDENIIENYYKNAQ |
| | ISASKGNNKVIKKYQKKVIECYIGY |
| UPBN01.1 | MGNLFGHKRWYEVRDKEDYKIRRKVKVKRNYDGNKYILNINENNNKEKIDNNKFIREFVNYKKNDN |
| SEQ ID NO: | VLREYKRKFHAGNILFKLKGKEKIKRIENNDDFLETEEVVLYIEVYGKSEKLKALGITKKKIIDEAIRQRI |
| 4189 | TKDDKKIEIKRQENKKKIEINIRDKCTNKTVDDCSVILRIIENDELETKKSIYEIFKNINMNLYKIIEKIIEN |
| | EAEKVFENRYYKEYLKEKLLEDNQINIILTNFMKIREKIESNPEIMGFVKFYFNVGGDKKKSENKKMFV |
| | EKILNINVDLTVEDIVDFIIGELKFYGIIKRIEKLQEKTVNRTDEDVKNTYKNT |
| IMG_ | MGNLFGYKRWYEVSDRGDNKIKRKVKIKRNYDGNKYILNINENNNKEKIENNEFIREFVNYKKNDNV |
| 3300007320 | LREFKRKFHAGNILFKLKGNKRSIGDSNDFLKTEEIILDKEVYGQSEKLRNEKGITKQDILKEIIDKGIDK |
| SEQ ID NO: | SNDKILVKTKLGKEITINFTDEDKKNKKEYQITLKIIPENELKIKREVYNVFKIINMNLYQIIKGIIENKEIF |
| 4190 | KNRYYDEILKEKLSKNNQIINTLTNLNKIRKEIRDNRDNIIGFVKFYLNVSGDKKKSENKKMFVEKILNI |
| | NVDLTVEDIVDFIVKELKFWNI |
| UPVO01.1 | MGNLFGYKKWYKVDKTIEKDGKTNTIKKEVRIKRNYLTDRYILNTNNKDKNNINNGDFVDQFIEYKT |
| SEQ ID NO: | KNDAFKKFTKKFHMGNILFKLKGNKRSIEDTNGFLKTEEIILDKEVYGQSEKLRNEKGITKQDILKEIID |
| 4191 | KGIDKSNDKILVKTKFGKEITINFTDEDKKNKNEYQITLKIIPENELKIKREVYNVFKIINMNLYQIIKGIIE |
| | NKEIFKNRYYDEILKEKLSKNNQIINTLTNLNKIRKEIRDNRDNIIGFVKFYLNVSGDKKKSENKKMFVE |
| | KILNTNVDLTVEDIVDFIVKELKFWNITKRIEKVKEFNNEFLENRRNRTYIKSYVLLDKHEKFKRDREN |
| | KKDIIVKSFIKDIKNNTMEQKINQILRKFKIKELTKKLDEAGIAYGIYYPVPLHLQKVYKNLGYKEGTLP |
| | NAEYLSKRTIAIPVDPELTEEEKEYIVDFLNNLDL |
| OEAE01.1 | LFLWIIEEVIYEIIKLYKNLKEEIIMGNLFGYKRWYEVRDKEDYKIKRKVKVKRNYDGNKYILNINENN |
| SEQ ID NO: | NKEKIDDNKFIREFVNYKKNDNVLIEFKRKFHAGNILFKLKGNKRSIEDSNGFLETGEIILDKEVYGQSE |
| 4192 | KLRNEKGITKQDILKEIIDKGIDKSNDKILVKTKFGKEITINFTDEDKKNKNEYQITLKIIPENELKIKREV |
| | YDVFKMINMDLYQIIKEIIENEVEKVFKNRYYEEHLKEKLLEDNQINVILTNFMKIREKIKSNPEIMGFIK |
| | FYLNVDGDKKKSENKKMFVEKILNINVDLTEEDIVDFIVKELKFWNITKRIEKLQEKKADRTDEDIKKT |
| | YINTYISLDKHEKFKKYDRNKKDTIVKSFIKDIKDNTMKQKINQILRKFKIEELIDKLRIENKNFDTEIFRI |
| | FKDHYQEIFSSEKFEEKSDEEKELYKIIYRYLKGRIEKILINEEKIKTKELKINKILDEKKLSEKVLKRVKQ |
| | YTLEHIMYLGKLRHNDIVKITVNTDDFSRLHAKEELDLELITFFASINMELNKIFEINKEKNDF |
| UPJQ01.1 | LFLWIIEEVIYEIIKLYKNLKEEIIMGNLFGYKKWYKVDKIIKDKKGKESTIKQEVRIKRNYTVDRYTLNT |
| SEQ ID NO: | NNKEKNNINNEDFVNQFIEYKTNNDIFRKFTRKFHAGNILFKLKGNKRSIEDSNGFLKTEEIILDKEVYG |
| 4193 | QSEKLRNEKGITKQDILKEIIDKGIDKSNDKILVKTKFGKEITINFTDEDKKNKNEYQITLKIIPENELKIK |
| | REVYNIFKIINMNLYQIIKEIIENEVQKVFKNRYYEEYLKEKLLEDNQINVILTNFMEIREKIKSNLEIMGF |
| | VKFYLNVGGDKKKSENKKMFVEKILNINVDLTVEDIVDFIVKELKFWNITKRIEKVKEFNNKSLENRRN |
| | RTYIKSYVLLDKHEKFK |
| OECA01.1 | MGNLFGYKKWYKVDKIIKDKKGKKSTIKQEVRIKRNYLTNRYILNTNNKDKNNINNEDFVDQFIEYKT |
| SEQ ID NO: | KNDIFEKFTRKFHMGNILFKLKGNKRSIEDSNDFLKTEEVVLDKEIYGQSEKLRNEKGITKQGILKEIIDK |
| 4194 | GINESNDSILIKTKFGKEIKINFTDESKKNKNEYQITLKVIPENELKIKREVYDVFKMINMDLYQIIKEIIEN |
| | EVQKVFKNRYYEEYLREKLLEDNQINVILTNFMKIREKTKSNSEIMGFVKFYLNVGGDKKKSENKKMF |
| | VEKILNINVDLTEEDIVDFIVKELKFYGIIKRIEKLQEKKADRTDKDIKKTYINTYVSLDKHEKFKKYNR |
| | NKKDTIVKSFIKDIKDNTMKQKINQILRKFKIEELINKLRIENKNFDTEIFRIFKEHYQEIFNSEKFEEKSDE |
| | EKELYKIIYRYLKGRIEKILINEQKVRLKKMEKIEVEKILNESILSEKILKRVKQYTLEHVMYLGKLVHN |
| | DIDRSIVNTNDFSRLHAKEELDLELITFFASTNMELNKIFSRENINNDENIDFFGGDREKNYVLDKKNLN |
| | SKIEIIRDLDFIDNKNSITNNFISKFTKIGTNERNRILHASSKERDLQGTQDDYNKVINIIQNLKISDEEVSK |
| | ALNLDVVFKDKKNIITKINDIEISEENNSIIKYLPSFSKVLPEILNLYKNKNKNNPFDTTETERIMLNALIY |
| | VNKELYKKLILEKNLEENESKNKFLKELKKNLGGTDEIDENIIESYYKNTQISASKGNNKAIKKYQKKII |
| | ECYIKYLEENYRELFDFSDFKMNIQEIKKQIKEINDNKTYKRITIKTSDKSIVINNDFEYIISIFALLNNNIFI |
| | NKIRNRFFSTSVWLNTSEYQNIIDILDEIMQLNTLRNECITENWNL |
| UPBH01.1 | LKGNKRSIEDSNDFLKTEEVVLDKEIYGQSEKLRNEKGITKKDILKEINKQKIDNSVKKISMNTNSGKTI |
| SEQ ID NO: | VINFSDKLKKDKDDYQITLNIISEDELERKRKIYDIFKMINMDLYQIIKEIIENEVQKVFKNRYYEEHLRE |
| 4195 | KLLKDDKIDVILTNFMKIREKIENNPEIMGFIKFYLNVGGDKKKSENKKIFVEKILNTNVDLTVEDVVDF |
| | IVKELKFWNITKRIEKVKEFNNKSLENKRNRTYIKSYVQLDKHEKFKIERENKKDKIVKLFVKDIHNNT |
| | MEEKINQILNKFKIKELIEKLKENTENKNFDTEIFGIFKTHYQNIFSSEKFSNKSDEEKELYKIIYRYLKGR |
| | IEKILINEEKVRLKKMEKIEIEKILNESILSEKILKRVKQYTLEHIMYLGKLIHNKINMATVNTNDFSRLHA |
| | KEELDLELITFFASTNMELNKIFNGKEKVTDFFGSNLNGQKITLKEKVPSFKLNILKKLNFINNENNIDEK |
| | LSHFYSFQKEGYLLRNKILHNSYGNIQETKNLEEKYKNVKNLIDELKVSDEEISKSLNLDVIFEGKNNIII |
| | EINKLQTGKYKDKKYLPSFSKIVPEIMRKFREINKDKSFDIESEKIILNAVQYVNKILYEKITSNEENEFIK |
| | TLPDKLVKKNNNKENKNSLSIEEYYKNAQVSSSK |
| UPFW01.1 | MGNLFGYKKWYKVDKTIEKDGKTNTVKKEVRIKRNYLTDRYILNTNNKDKNNINNGDFVDQFIEYKT |
| SEQ ID NO: | NNDIFRKFTRKFHMGNILFKLKAKESIKKAKESIKKIESYNNFLEKEKAILEIEIYQQSEKLIEEENITKKDI |
| 4196 | IDKAIKEKITEDSNEIKMQIKSKENKLKEIKISINKETEEYHIKLRSINNDELNLKREIYEILKSINANLYIIT |
| | KNAISNADFKKRNYENFLRENIMEHLKKNIGEKSKITFLKSLSNSLKKLQGNIKENDEIINFIKYYSNING |
| | CKTVSENKKNFLEKILNTEVSVSENDIIDFIIGELKFYGIIKRIEKLQEKTVNRTDKDIKNTYKNTYVLLD |
| | KHEKFKKYNRNPKDIIVKSFIKDIKDNTMEQKINQILRKFKIEELIKKLKMEDKNFDTEIFGIFKVHYQEI |
| | FSSEKFEKKSDEEKELYKHYRYLKGRIEKILVNEQKVRLKKMEKIEIEKILNESILSEKILKRVKQYTLEH |
| | IMYLGKLRHNDIVKITVNTDDFSRLHAKEELDLELITFFASTNMELNKIFEINKEKNDFFGDSFKINDTK |
| | VLLKNEVTSSKLYILKNLNFIDNENKVKKEEFISKFIT |
| UPLQ01.1 | MKDKLDMLNKNEAVTDLRFGIVSEKYEKGITSFDYERIKAEEELDRNIAAYVTFAADIFAKSVIKSDYR |
| SEQ ID NO: | TKKDNNSDVLQYSDKEFRNSEVIRDNAEKNILQYWGGYSRWGTENSKLPENNKVSDFGKLDVSDLCI |
| 4197 | DIKKHLAGIRNSSVHYTTKIKNESAADGSNVKILFEKDLADINIIYADKYYSNNVWMFYSLEDINKLIAF |
| | LYKEKRVIRQVQIPSFSRILKRKAMQDVINEIFKDDFDENIVNPELKEKYRNSLYFMLKEIYYNAFIIQPE |
| | LKEKFKDKIKTMKSELYNKLKTIDKKEYKALYCMLSNEESALKNFADRIYSVDGDNVSFADICKILMT |
| | DYNMQNQEKKNIESMEQKKKNKGKDENYKHFPLLLHKVLKELFIEYLKQTHELEFLRNNICIKSDVTM |
| | ESFESQIKGVEIYKDLKEKIDKNNSLLDWYVIAHFLMPKQLNHLIGNIKNYIQFATNIDKRAESVKNLTE |
| | SGMVKRIQYYDDIVRTLEFSAQYIGKISNNINDYFNSEDEYVSFLSKYVGFINDKSEDVLTELKDFCREK |
| | INNGSQIIGIYYGGDNVIINRNVIYAQMYSNAEVFSNIYKKVTKNDIINYYEKQNDLKDVFKSGVCQNE |
| | DEQRSLCEFQQLKNRIELTELSTYADMVNDFMAQFVEWAYLRERDLMYFQLGIHYIRLFYSDNVLDEK |
| | YHKLSDNVIDIEEGALLYQIVAMYDYELRIFETDNSGNAKRIGQGGPGKSIPVFLKKYCNVTDVYECGL |
| | ELFEDINQHEHIIRFRNDIAHMRYMSNQAMNIMSIVSNIYKSFFVYDTKLKKSISLVFKNILMRYGVIAD |
| | LVFSYNNKNQSIKDDMDINIINIKNLKSDKYVYKIVNEDIVKQVEIEIRNQVFLEQLHNLLYFSR |
| UPEL01.1 | MKDKLDMLNKNEAVTDLRFGIVSEKYEKGITSFDYERIKAEEELDRNIAAYVTFAADIFAKSVIKSDYR |
| SEQ ID NO: | TKKDNNSDVLQYSDKEFRNSEVIRDNAEKNILQYWGGYSRWGTENSKLPENNKVSDFGKLDVSDLCI |
| 4198 | DIKKHLAGIRNSSVHYTTKIKNESAADGSNVKILFEKDLADINIIYADKYYSNNVWMFYSLEDINKLIAF |
| | LYKEKRVIRQVQIPSFSRILKRKAMQDVINEIFKDDFDENIVNPELKEKYRNSLYFMLKEIYYNAFIIQPE |
| | LKEKFKDKIKTMKSELYNKLKTIDKKEYKALYCMLSNEESALKNFADRIYSVDGDNVSFADICKILMT |
| | DYNMQNQEKKNIESMEQKKKNKGKDENYKHFPLLLHKVLKELFIEYLKQTHELEFLRNNICIKSDVTM |
| | ESFESQIKGVEIYKDLKEKIDKNNSLLDWYVIAHFLMPKQLNHLIGNIKNYIQFATNIDKRAESVKNLTE |
| | SGMVKRIQYYDDIVRTLEFSAQYIGKISNNINDYFNSEDEYVSFLSKYVGFINDKSEDVLTELKDFCREK |
| | INNGSQIIGIYYGGDNVIINRNVIYAQMYSNAEVFSNIYKKVTKNDIINYYEKQNDLKDVFKSGVCQNE |
| | DEQRSLCEFQQLKNRIELTELSTYADMVNDFMAQFVEWAYLRERDLMYFQLGIHYIRLFYSDNVLDEK |
| | YHKLSDNVIDIEEGALLYQIVAMYDYELRIFETDNSGNAKRIGQGGPGKSIPVFLKKYCNVTDVYECGL |
| | ELFEDINQHEHIIRFRNDIAHMRYMSNQAMNIMSIVSNIYKSFFVYDTKLKKSISLVFKNILMRYGVIAD |
| | LVFSYNNKNQSIKDDMDINIINIKNLKSDKYVYKIVNEDIVKQVEIEIRNQVFLEQLHNLLYFSR |
| OVYE01.1 | MKDKLDMLNKNEAVTDLRFGIVSEKYEKGITSFDYERIKAEEELDRNIAAYVTFAADIFAKSVIKSDYR |
| SEQ ID NO: | TKKDNNSDVLQYSDKEFRNSEVIRDNAEKNILQYWGGYSRWGTENSKLPENNKVSDFGKLDVSDLCI |
| 4199 | DIKKHLAGIRNSSVHYTTKIKNESAADGSNVKILFEKDLADINIIYADKYYSNNVWMFYSLEDINKLIAF |
| | LYKEKRVIRQVQIPSFSRILKRKAMQDVINEIFKDDFDENIVNPELKEKYRNSLYFMLKEIYYNAFIIQPE |
| | LKEKFKDKIKTMKSELYNKLKTIDKKEYKALYCMLSNEESALKNFADRIYSVDGDNVSFADICKILMT |
| | DYNMQNQEKKNIESMEQKKKNKGKDENYKHFPLLLHKVLKELFIEYLKQTHELEFLRNNICIKSDVTM |
| | ESFESQIKGVEIYKDLKEKIDKNNSLLDWYVIAHFLMPKQLNHLIGNIKNYIQFATNIDKRAESVKNLTE |
| | SGMVKRIQYYDDIVRTLEFSAQYIGKISNNINDYFNSEDEYVSFLSKYVGFINDKSEDVLTELKDFCREK |
| | INNGSQIIGIYYGGDNVIINRNVIYAQMYSNAEVFSNIYKKVTKNDIINYYEKQNDLKDVFKSGVCQNE |
| | DEQRSLCEFQQLKNRIELTELSTYADMVNDFMAQFVEWAYLRERDLMYFQLGIHYIRLFYSDNVLDEK |
| | YHKLSDNVIDIEEGALLYQIVAMYDYELRIFETDNSGNAKRIGQGGPGKSIPVFLKKYCNVTDVYECGL |
| | ELFEDINQHEHIIRFRNDIAHMRYMSNQAMNIMSIVSNIYKSFFVYDTKLKKSISLVFKNILMRYGVIAD |
| | LVFSYNNKNQSIKDDMDINIINIKNLKSDKYVYKIVNEDIVKQVEIEIRNQVFLEQLHNLLYFSR |
| OOCS01.1 | MKDKLDMLNKNEAVTDLRFGIVSEKYEKGITSFDYERIKAEEELDRNIAAYVTFAADIFAKSVIKSDYR |
| SEQ ID NO: | TKKDNNSDVLQYSDKEFRNSEVIRDNAEKNILQYWGGYSRWGTENSKLPENNKVSDFGKLDVSDLCI |
| 4200 | DIKKHLAGIRNSSVHYTTKIKNESAADGSNVKILFEKDLADINIIYADKYYSNNVWMFYSLEDINKLIAF |
| | LYKEKRVIRQVQIPSFSRILKRKAMQDVINEIFKDDFDENIVNPELKEKYRNSLYFMLKEIYYNAFIIQPE |
| | LKEKFKDKIKTMKSELYNKLKTIDKKEYKALYCMLSNEESALKNFADRIYSVDGDNVSFADICKILMT |
| | DYNMQNQEKKNIESMEQKKKNKGKDENYKHFPLLLHKVLKELFIEYLKQTHELEFLRNNICIKSDVTM |
| | ESFESQIKGVEIYKDLKEKIDKNNSLLDWYVIAHFLMPKQLNHLIGNIKNYIQFATNIDKRAESVKNLTE |
| | SGMVKRIQYYDDIVRTLEFSAQYIGKISNNINDYFNSEDEYVSFLSKYVGFINDKSEDVLTELKDFCREK |
| | INNGSQIIGIYYGGDNVIINRNVIYAQMYSNAEVFSNIYKKVTKNDIINYYEKQNDLKDVFKSGVCQNE |
| | DEQRSLCEFQQLKNRIELTELSTYADMVNDFMAQFVEWAYLRERDLMYFQLGIHYIRLFYSDNVLDEK |
| | YHKLSDNVIDIEEGALLYQIVAMYDYELRIFETDNSGNAKRIGQGGPGKSIPVFLKKYCNVTDVYECGL |
| | ELFEDINQHEHIIRFRNDIAHMRYMSNQAMNIMSIVSNIYKSFFVYDTKLKKSISLVFKNILMRYGVIAD |
| | LVFSYNNKNQSIKDDMDINIINIKNLKSDKYVYKIVNEDIVKQVEIEIRNQVFLEQLHNLLYFSR |
| OLGD01.1 | MKDKLDMLNKNEAVTDLRFGIVSEKYEKGITSFDYERIKAEEELDRNIAAYVTFAADIFAKSVIKSDYR |
| SEQ ID NO: | TKKDNNSDVLQYSDKEFRNSEVIRDNAEKNILQYWGGYSRWGTENSKLPENNKVSDFGKLDVSDLCI |
| 4201 | DIKKHLAGIRNSSVHYTTKIKNESAADGSNVKILFEKDLADINIIYADKYYSNNVWMFYSLEDINKLIAF |
| | LYKEKRVIRQVQIPSFSRILKRKAMQDVINEIFKDDFDENIVNPELKEKYRNSLYFMLKEIYYNAFIIQPE |
| | LKEKFKDKIKTMKSELYNKLKTIDKKEYKALYCMLSNEESALKNFADRIYSVDGDNVSFADICKILMT |
| | DYNMQNQEKKNIESMEQKKKNKGKDENYKHFPLLLHKVLKELFIEYLKQTHELEFLRNNICIKSDVTM |
| | ESFESQIKGVEIYKDLKEKIDKNNSLLDWYVIAHFLMPKQLNHLIGNIKNYIQFATNIDKRAESVKNLTE |
| | SGMVKRIQYYDDIVRTLEFSAQYIGKISNNINDYFNSEDEYVSFLSKYVGFINDKSEDVLTELKDFCREK |
| | INNGSQIIGIYYGGDNVIINRNVIYAQMYSNAEVFSNIYKKVTKNDIINYYEKQNDLKDVFKSGVCQNE |
| | DEQRSLCEFQQLKNRIELTELSTYADMVNDFMAQFVEWAYLRERDLMYFQLGIHYIRLFYSDNVLDEK |
| | YHKLSDNVIDIEEGALLYQIVAMYDYELRIFETDNSGNAKRIGQGGPGKSIPVFLKKYCNVTDVYECGL |
| | ELFEDINQHEHIIRFRNDIAHMRYMSNQAMNIMSIVSNIYKSFFVYDTKLKKSISLVFKNILMRYGVIAD |
| | LVFSYNNKNQSIKDDMDINIINIKNLKSDKYVYKIVNEDIVKQVEIEIRNQVFLEQLHNLLYFSR |
| OVFU01.1 | MKDKLDMLNKNEAVTDLRFGIVSEKYEKGITSFDYERIKAEEELDRNIAAYVTFAADIFAKSVIKSDYR |
| SEQ ID NO: | TKKDNNSDVLQYSDKEFRNSEVIRDNAEKNILQYWGGYSRWGTENSKLPENNKVSDFGKLDVSDLCI |
| 4202 | DIKKHLAGIRNSSVHYTTKIKNESAADGSNVKILFEKDLADINIIYADKYYSNNVWMFYSLEDINKLIAF |
| | LYKEKRVIRQVQIPSFSRILKRKAMQDVINEIFKDDFDENIVNPELKEKYRNSLYFMLKEIYYNAFIIQPE |
| | LKEKFKDKIKTMKSELYNKLKTIDKKEYKALYCMLSNEESALKNFADRIYSVDGDNVSFADICKILMT |
| | DYNMQNQEKKNIESMEQKKKNKGKDENYKHFPLLLHKVLKELFIEYLKQTHELEFLRNNICIKSDVTM |
| | ESFESQIKGVEIYKDLKEKIDKNNSLLDWYVIAHFLMPKQLNHLIGNIKNYIQFATNIDKRAESVKNLTE |
| | SGMVKRIQYYDDIVRTLEFSAQYIGKISNNINDYFNSEDEYVSFLSKYVGFINDKSEDVLTELKDFCREK |
| | INNGSQIIGIYYGGDNVIINRNVIYAQMYSNAEVFSNIYKKVTKNDIINYYEKQNDLKDVFKSGVCQNE |
| | DEQRSLCEFQQLKNRIELTELSTYADMVNDFMAQFVEWAYLRERDLMYFQLGIHYIRLFYSDNVLDEK |
| | YHKLSDNVIDIEEGALLYQIVAMYDYELRIFETDNSGNAKRIGQGGPGKSIPVFLKKYCNVTDVYECGL |
| | ELFEDINQHEHIIRFRNDIAHMRYMSNQAMNIMSIVSNIYKSFFVYDTKLKKSISLVFKNILMRYGVIAD |
| | LVFSYNNKNQSIKDDMDINIINIKNLKSDKYVYKIVNEDIVKQVEIEIRNQVFLEQLHNLLYFSR |
| OLGB01.1 | MKDKLDMLNKNEAVTDLRFGIVSEKYEKGITSFDYERIKAEEELDRNIAAYVTFAADIFAKSVIKSDYR |
| SEQ ID NO: | TKKDNNSDVLQYSDKEFRNSEVIRDNAEKNILQYWGGYSRWGTENSKLPENNKVSDFGKLDVSDLCI |
| 4203 | DIKKHLAGIRNSSVHYTTKIKNESAADGSNVKILFEKDLADINIIYADKYYSNNVWMFYSLEDINKLIAF |
| | LYKEKRVIRQVQIPSFSRILKRKAMQDVINEIFKDDFDENIVNPELKEKYRNSLYFMLKEIYYNAFIIQPE |
| | LKEKFKDKIKTMKSELYNKLKTIDKKEYKALYCMLSNEESALKNFADRIYSVDGDNVSFADICKILMT |
| | DYNMQNQEKKNIESMEQKKKNKGKDENYKHFPLLLHKVLKELFIEYLKQTHELEFLRNNICIKSDVTM |
| | ESFESQIKGVEIYKDLKEKIDKNNSLLDWYVIAHFLMPKQLNHLIGNIKNYIQFATNIDKRAESVKNLTE |
| | SGMVKRIQYYDDIVRTLEFSAQYIGKISNNINDYFNSEDEYVSFLSKYVGFINDKSEDVLTELKDFCREK |
| | INNGSQIIGIYYGGDNVIINRNVIYAQMYSNAEVFSNIYKKVTKNDIINYYEKQNDLKDVFKSGVCQNE |
| | DEQRSLCEFQQLKNRIELTELSTYADMVNDFMAQFVEWAYLRERDLMYFQLGIHYIRLFYSDNVLDEK |
| | YHKLSDNVIDIEEGALLYQIVAMYDYELRIFETDNSGNAKRIGQGGPGKSIPVFLKKYCNVTDVYECGL |
| | ELFEDINQHEHIIRFRNDIAHMRYMSNQAMNIMSIVSNIYKSFFVYDTKLKKSISLVFKNILMRYGVIAD |
| | LVFSYNNKNQSIKDDMDINIINIKNLKSDKYVYKIVNEDIVKQVEIEIRNQVFLEQLHNLLYFSR |
| UPEO01.1 | MKDKLDMLNKNEAVTDLRFGIVSEKYEKGITSFDYERIKAEEELDRNIAAYVTFAADIFAKSVIKSDYR |
| SEQ ID NO: | TKKDNNSDVLQYSDKEFRNSEVIRDNAEKNILQYWGGYSRWGTENSKLPENNKVSDFGKLDVSDLCI |
| 4204 | DIKKHLAGIRNSSVHYTTKIKNESAADGSNVKILFEKDLADINIIYADKYYSNNVWMFYSLEDINKLIAF |
| | LYKEKRVIRQVQIPSFSRILKRKAMQDVINEIFKDDFDENIVNPELKEKYRNSLYFMLKEIYYNAFIIQPE |
| | LKEKFKDKIKTMKSELYNKLKTIDKKEYKALYCMLSNEESALKNFADRIYSVDGDNVSFADICKILMT |
| | DYNMQNQEKKNIESMEQKKKNKGKDENYKHFPLLLHKVLKELFIEYLKQTHELEFLRNNICIKSDVTM |
| | ESFESQIKGVEIYKDLKEKIDKNNSLLDWYVIAHFLMPKQLNHLIGNIKNYIQFATNIDKRAESVKNLTE |
| | SGMVKRIQYYDDIVRTLEFSAQYIGKISNNINDYFNSEDEYVSFLSKYVGFINDKSEDVLTELKDFCREK |
| | INNGSQIIGIYYGGDNVIINRNVIYAQMYSNAEVFSNIYKKVTKNDIINYYEKQNDLKDVFKSGVCQNE |
| | DEQRSLCEFQQLKNRIELTELSTYADMVNDFMAQFVEWAYLRERDLMYFQLGIHYIRLFYSDNVLDEK |
| | YHKLSDNVIDIEEGALLYQIVAMYDYELRIFETDNSGNAKRIGQGGPGKSIPVFLKKYCNVTDVYECGL |
| | ELFEDINQHEHIIRFRNDIAHMRYMSNQAMNIMSIVSNIYKSFFVYDTKLKKSISLVFKNILMRYGVIAD |
| | LVFSYNNKNQSIKDDMDINIINIKNLKSDKYVYKIVNEDIVKQVEIEIRNQVFLEQLHNLLYFSR |
| OPMQ01.1_ | MYSNAEVFSNIYKKVTKNDIINYYEKQNDLKDVFKSGVCQNEDEQRSLCEFQQLKNRIELTELSTYAD |
| 2 | MVNDFMAQFVEWAYLRERDLMYFQLGIHYIRLFYSDNVLDEKYHKLSDNVIDIEEGALLYQIVAMYD |
| SEQ ID NO: | YELRIFETDNSGNAKRIGQGGPGKSIPVFLKKYCNVTDVYECGLELFEDINQHEHIIRFRNDIAHMRYMS |
| 4205 | NQAMNIMSIVSNIYKSFFVYDTKLKKSISLVFKNILMRYGVIADLVFSYNNKNQSIKDDMDINIINIKNL |
| | KSDKYVYKIVNEDIVKQVEIEIRNQVFLEQLHNLLYFSR |
| UYCD01.1 | MKDKLDMLNKNEAVTDLRFGIVSEKYEKGITSFDYERIKAEEELDRNIAAYVTFAADIFAKSVIKSDYR |
| SEQ ID NO: | TKKDNNSDVLQYSDKEFRNSEVIRDNAEKNILQYWGGYSRWGTENSKLPENNKVSDFGKLDVSDLCI |
| 4206 | DIKKHLAGIRNSSVHYTTKIKNESAADGSNVKILFEKDLADINIIYADKYYSNNVWMFYSLEDINKLIAF |
| | LYKEKRVIRQVQIPSFSRILKRKAMQDVINEIFKDDFDENIVNPELKEKYRNSLYFMLKEIYYNAFIIQPE |
| | LKEKFKDKIKTMKSELYNKLKTIDKKEYKALYCMLSNEESALKNFADRIYSVDGDNVSFADICKILMT |
| | DYNMQNQEKKNIESMEQKKKNKGKDENYKHFPLLLHKVLKELFIEYLKQTHELEFLRNNICIKSDVTM |
| | ESFESQIKGVEIYKDLKEKIDKNNSLLDWYVIAHFLMPKQLNHLIGNIKNYIQFATNIDKRAESVKNLTE |
| | SGMVKRIQYYDDIVRTLEFSAQYIGKISNNINDYFNSEDEYVSFLSKYVGFINDKSEDVLTELKDFCREK |
| | INNGSQIIGIYYGGDNVIINRNVIYAQMYSNAEVFSNIYKKVTKNDIINYYEKQNDLKDVFKSGVCQNE |
| | DEQRSLCEFQQLKNRIELTELSTYADMVNDFMAQFVEWAYLRERDLMYCQLGIHYIRLFYSDNVLDE |
| | KYHKLSDNVIDIEEGALLYQIVAMYDYELRIFETDNSGNAKRIGQGGPGKSIPVFLKKYCNVTDVYECG |
| | LELFEDINQHEHIIRFRNDIAHMRYMSNQAMNIMSIVSNIYKSFFVYDTKLKKSISLVFKNILMRYGVIA |
| | DLVFSYNNKNQSIKDDMDINIINIKNLKSDKYVYKIVNEDIVKQVEIEIRNQVFLEQLHNLLYFSR |
| ORNQ01.1 | MPKQLNHLIGNIKNYIQFATNIDKRAESVKNLTESGMVKRIQYYDDIVRTLEFSAQYIGKISNNINDYFN |
| SEQ ID NO: | SEDEYVSFLSKYVGFINDKSEDVLTELKDFCREKINNGSQIIGIYYGGDNVIINRNVTYAQMYSNAEVFS |
| 4207 | NIYKKVTKNDIINYYEKQNDLKDVFKSGVCQNEDEQRSLCEFQQLKNRIELTELSTYADMVNDFMAQF |
| | VEWAYLRERDLMYFQLGIHYIRLFYSDNVLDEKYHKLSDNVIDIEEGALLYQIVAMYDYELRIFETDNS |
| | GNAKRIGQGGPGKSIPVFLKKYCNGTDVYECGLELFEDINQHEHIIRFRNDIAHMRYMSNQAMNIMSIV |
| | SNIYKSFFVYDTKLKKSISLVFKNILMRYGVIADLVFSYNNKNQSIKDDMDINIINIKNLKSDKYVYKIV |
| | NEDIVKQVEIEIRNQVFLEQLHNLLYFSR |
| ULRY01.1 | MKDKLDMLNKNEAVTDLRFGIVSEKYEKGITSFDYERIKAEEELDRNIAAYVTFAADIFAKSVIKSDYR |
| SEQ ID NO: | TKKDNNSDVLQYSDKEFRNSEVIRDNAEKNILQYWGGYSRWGTENSKLPENNKVSDFGKLDVSDLCI |
| 4208 | DIKKHLAGIRNSSVHYTTKIKNESAADGSNVKILFEKDLADINIIYADKYYSNNVWMFYSLEDINKLIAF |
| | LYKEKRVIRQVQIPSFSRILKRKAMQDVINEIFKDDFDENIVNPELKEKYRNSLYFMLKEIYYNAFIIQPE |
| | LKEKFKDKIKTMTSELYNKLKTIDKKEYKALYCMLSNEESALKNFADRIYSVDGDNVSFADICKILMT |
| | DYNMQNQEKKNIESMEQKKKNKGKDENYKHFPLLLHKVLKELFIEYLKQTHELEFLRNNICIKSDVTM |
| | ESFESQIKGVEIYKDLKEKIDKNNSLLDWYVIAHFLMPKQLNHLIGNIKNYIQFATNIDKRAESVKNLTE |
| | SGMVKKIQYYDDIVRTLEFSAQYIGKISNNINDYFNSEDEYVSFLSKYVGFINDKSEDVLTELKDFCREK |
| | INNGSQIIGIYYGGDNVIINRNVIYAQMYSNAEVFSNIYKKVTKNDIINYYEKQNDLKDVFKSGVCQNE |
| | GEQRSLCEFQQLKNRIELTELSTYADMVNDFMAQFVEWAYLRERDLMYFQLGIHYIRLFYSDNVLDEK |
| | YHKLSDNVIDIEEGALLYQIVAMYDYELRIFETDNSGNAKRIGQGGPGKSIPVFLKKYCNGTDVYECGL |
| | ELFEDINQHEHIIRFRNDIAHMRYMSNQAMNIMSIVSNIYKSFFVYDTKLKKSISLVFKNILMRYGVIAD |
| | FVFSYNNKNQSIKDDMDINIINIKNLKSDKYVYKIVNEDIVKQVEIEIRNQVFLEQLHNLLYFSR |
| ORQX01.1 | MKDKLDMLNKNEAVTDLRFGIVSEKYEKGITSFDYERIKAEEELDRNIAAYVTFAADIFAKSVIKSDYR |
| SEQ ID NO: | TKKDNNSDVLQYSDKEFRNSEVIRDNAEKNILQYWGGYSRWGTENSKLPENNKVSDFGKLDVSDLCI |
| 4209 | DIKKHLAGIRNSSVHYTTKIKNESAADGSNVKILFEKDLADINIIYADKYYSNNVWMFYSLEDINKLIAF |
| | LYKEKRVIRQVQIPSFSRILKRKAMQDVINEIFKDDFDENIVNPELKEKYRNSLYFMLKEIYYNAFIIQPE |
| | LKEKFKDKIKTMTSELYNKLKTIDKKEYKALYCMLSNEESALKNFADRIYSVDGDNVSFADICKILMT |
| | DYNMQNQEKKNIESMEQKKKNKGKDENYKHFPLLLHKVLKELFIEYLKQTHELEFLRNNICIKSDVTM |
| | ESFESQIKGVEIYKDLKEKIDKNNSLLDWYVIAHFLMPKQLNHLIGNIKNYIQFATNIDKRAESVKNLTE |
| | SGMVKKIQYYDDIVRTLEFSAQYIGKISNNINDYFNSEDEYVSFLSKYVGFINDKSEDVLTELKDFCREK |
| | INNGSQIIGIYYGGDNVIINRNVIYAQMYSNAEVFSNIYKKVTKNDIINYYEKQNDLKDVFKSGVCQNE |
| | GEQRSLCEFQQLKNRIELTELSTYADMVNDFMAQFVEWAYLRERDLMYFQLGIHYIRLFYSDNVLDEK |
| | YHKLSDNVIDIEEGALLYQIVAMYDYELRIFETDNSGNAKRIGQGGPGKSIPVFLKKYCNGTDVYECGL |
| | ELFEDINQHEHIIRFRNDIAHMRYMSNQAMNIMSIVSNIYKSFFVYDTKLKKSISLVFKNILMRYGVIAD |
| | FVFSYNNKNQSIKDDMDINIINIKNLKSDKYVYKIVNEDIVKQVEIEIRNQVFLEQLHNLLYFSR |
| OXAA01.1 | MKDKLDMLNKNEAITDLRFGIVSEKYEKGITSFDYERIKAEEELDRNIAAYVTFAADIFAKSVIKSDYRT |
| SEQ ID NO: | KKDNNSDVLQYSDKEFRNSEVIRDNAEKNILQYWGGYSRWGTENSKLPENNKVSDFGKLDVSDLCIDI |
| 4210 | KKHLAGIRNSSVHYTTKIKNESAADGSNVKILFEKDLADINIIYADKYYSNNVWMFYSLEDINKLIAFL |
| | YKEKRVIRQVQIPSFSRILKRKAMQDVINEIFKDDFDENIVNPELKEKYRNSLYFMLKEIYYNAFIIQPEL |
| | KEKFKDKIKTMTSELYNKLKTIDKKEYKALYCMLSNEESALKNFADRIYSVDGDNVSFADICKILMTD |
| | YNMQNQEKKNIESMEQKKKNKGKDENYKHFPLLLHKVLKELFIEYLKQTHELEFLRNNICIKSDVTME |
| | SFESQIKGVEIYKDLKEKIDKNNSLLDWYVIAHFLMPKQLNHLIGNIKNYIQFATNIDKRAESVKNLTES |
| | GMVKKIQYYDDIVRTLEFSAQYIGKISNNINDYFNSEDEYVSFLSKYVGFINDKSEDVLTELKDFCREKI |
| | NNGSQIIGIYYGGDNVIINRNVIYAQMYSNAEVFSNIYKKVTKNDIINYYEKQNDLKDVFKSGVCQNEG |
| | EQRSLCEFQQLKNRIELTELSTYADMVNDFMAQFVEWAYLRERDLMYFQLGIHYIRLFYSDNVLDEKY |
| | HKLSDNVIDIEEGALLYQIVAMYDYELRIFETDNSGNAKRIGQGGPGKSIPVFLKKYCNGTDVYECGLE |
| | LFEDINQHEHIIRFRNDIAHMRYMSNQAMNIMSIVSNIYKSFFVYDTKLKKSISLVFKNILMRYGVIADF |
| | VFSYNNKNQSIKDDMDINIINIKNLKSDKYVYKIVNEDIVKQVEIEIRNQVFLEQLHNLLYFSR |
| UZKN01.1 | LKNFADRIYSVDGDNVSFADICKILMTDYNMQNQEKKNIESMEQKKKNKGKDENYKHFPLLLHKVLK |
| SEQ ID NO: | ELFIEYLKQTHELEFLRNNICIKSDVTMESFESQIKGVEIYKDLKEKIDKNNSLLDWYVIAHFLMPKQLN |
| 4211 | HLIGNIKNYIQFATNIDKRAESVKNLTESGMVKRIQYYDDIVRTLEFSAQYIGKISNNINDYFNSEDEYV |
| | SFLSKYVGFINDKSEDVLTELKDFCREKINNGSQIIGIYYGGDNVIINRNVIYAQMYSNAEVFSNIYKKV |
| | TKNDIINYYEKQNDLKDVFKSGVCQNEDEQRSLCEFQQLKNRIELTELSTYADMVNDFMAQFVEWAY |
| | LRERDLMYFQLGIHYIRLFYSDNVLDEKYHKLSDNVIDIEEGALLYQIVAMYDYELRIFETDNSGNAKR |
| | IGQGGPGKSIPVFLKKYCNVTDVYECGLELFEDINQHEHIIRFRNDIAHMRYMSNQAMNIMSIVSNIYKS |
| | FFVYDTKLKKSISLVFKNILMRYGVIADLVFSYNNKNQSIKDDMDINIINIKNLKSDKYDIK |
| OOUM01.1 | MQGIFQKENTDKALKYGIYRHLPTYEKIIRNLVSFLSKYVGFINDKSEDVLTELKDFCREKINNGSQIIGI |
| SEQ ID NO: | YYGGDNVIINRNVIYAQMYSNAEVFSNIYKKVTKNDIINYYEKQNDLKDVFKSGVCQNEDEQRSLCEF |
| 4212 | QQLKNRIELTELSTYADMVNDFMAQFVEWAYLRERDLMYFQLGIHYIRLFYSDNVLDEKYHKLSDNV |
| | IDIEEGALLYQIVAMYDYELRIFETDNSGNAKRIGQGGPGKSIPVFLKKYCSGTDVYECGLELFEDINQH |
| | EHIIRFRNDIAHMRYMSNQAMNIMSIVSNIYKSFFAYDTKLKKSISLVFKNILMRYGVIADLVFSYNNKN |
| | QSIKDDMDINIINIKNLKSDKYVYKIVNEDIVKQVEIEIRNQVFLEQLHNLLYFSR |
| OPMQ01.1 | MKDKLDMLNKNEAVTDLRFGIVSEKYEKGITSFDYERIKAEEELDRNIAAYVTFAADIFAKSVIKSDYR |
| SEQ ID NO: | TKKDNNSDVLQYSDKEFRNSEVIRDNAEKNILQYWGGYSRWGTENSKLPENNKVSDFGKLDVSDLCI |
| 4213 | DIKKHLAGIRNSSVHYTTKIKNESAADGSNVKILFEKDLADINIIYADKYYSNNVWMFYSLEDINKLIAF |
| | LYKEKRVIRQVQIPSFSRILKRKAMQDVINEIFKDDFDENIVNPELKEKYRNSLYFMLKEIYYNAFIIQPE |
| | LKEKFKDKIKTMKSELYNKLKTIDKKEYKALYCMLSNEESALKNFADRIYSVDGDNVSFADICKILMT |
| | DYNMQNQEKKNIESMEQKKKNKGKDENYKHFPLLLHKVLKELFIEYLKQTHELEFLRNNICIKSDVTM |
| | ESFESQIKGVEIYKDLKEKIDKNNSLLDWYVIAHFLMPKQLNHLIGNIKNYIQFATNIDKRAESVKNLTE |
| | SGMVKRIQYYDDIVRTLEFSAQYIGKISNNINDYFNSEDEYVSFLSKYVGFINDKSEDVLTELKDFCREK |
| | INNGSQIWCATCQ |
| UXLM01.1 | MKDKLDMLNKNEAVTDLRFGIVSEKYEKGITSFDYERIKAEEELDRNIAAYVTFAADIFAKSVIKSDYR |
| SEQ ID NO: | TKKDNNSDVLQYSDKEFRNSEVIRDNAEKNILQYWGGYSRWGTENSKLPENNKVSDFGKLDVSDLCI |
| 4214 | DIKKHLAGIRNSSVHYTTKIKNESAADGSNVKILFEKDLADINIIYADKYYSNNVWMFYSLEDINKLIAF |
| | LYKEKRVIRQVQIPSFSRILKRKAMQDVINEIFKDDFDENIVNPELKEKYRNSLYFMLKEIYYNAFIIQPE |
| | LKEKFKDKIKTMKSELYNKLKTIDKKEYKALYCMLSNEESALKNFADRIYSVDGDNVSFADICKILMT |
| | DYNMQNQEKKNIESMEQKKKNKGKDENYKHFPLLLHKVLKELFIEYLKQTHELEFLRNNICIKSDVTM |
| | ESFESQIKGVEIYKDLKEKIDKNNSLLDWYVIAHFLMPKQLNHLIGNIKNYIQFATNIDKRAESVKNLTE |
| | SGMVKRIQYYDDIVRTLEFSAQYIGKISNNINDYFNSEDEYVSFLSKYVGFINDKSEDVLTELKDFCREK |
| | INNGSQIWCATCQ |
| CDTY01.1 | MKDKLDMLNKNEAVTDLRFGIVSEKYEKGITSFDYERIKAEEELDRNIAAYVTFAADIFAKSVIKSDYR |
| SEQ ID NO: | TKKDNNSDVLQYSDKEFRNSEVIRDNAEKNILQYWGGYSRWGTENSKLPENNKVSDFGKLDVSDLCI |
| 4215 | DIKKHLAGIRNSSVHYTTKIKNESAADGSNVKILFEKDLADINIIYADKYYSNNVWMFYSLEDINKLIAF |
| | LYKEKRVIRQVQIPSFSRILKRKAMQDVINEIFKDDFDENIVNPELKEKYRNSLYFMLKEIYYNAFIIQPE |
| | LKEKFKDKIKTMKSELYNKLKTIDKKEYKALYCMLSNEESALKNFADRIYSVDGDNVSFADICKILMT |
| | DYNMQNQEKKNIESMEQKKKNKGKDENYKHFPLLLHKVLKELFIEYLKQTHELEFLRNNICIKSDVTM |
| | ESFESQIKGVEIYKDLKEKIDKNNSLLDWYVIAHFLMPKQLNHLIGNIKNYIQFATNIDKRAESVKNLTE |
| | SGMVKRIQYYDDIVRTLEFSAQYIGKISNNINDYFNSEDEYVSFLSKYVGFINDKSEDVLTELKDFCREK |
| | INNGSQIWCATCQ |
| OGMB01.1 | LSYLAAKYIDLGKGVYHFTMKDKLDMLNKNEAVTDLRFGIVSEKYEKGITSFDYERIKAEEELDRNIA |
| SEQ ID NO: | AYVTFAADIFAKSVIKSDYRTKKDNNSDVLQYSDKEFRNSEVIRDNAEKNILQYWGGYSRWGTENSKL |
| 4216 | PENNKVSDFGKLDVSDLCIDIKKHLAGIRNSSVHYTTKIKNESAADGSNVKILFEKDLADINIIYADKYY |
| | SNNVWMFYSLEDINKLIAFLYKEKRVIRQVQIPSFSRILKRKAMQDVINEIFKDDFDENIVNPELKEKYR |
| | NSLYFMLKEIYYNAFIIQPELKEKFKDKIKTMKSELYNKLKTIDKKEYKALYCMLSNEESALKNFADRI |
| | YSVDGDNVSFA |
| OWET01.1 | MKDKLDMLNKNEAVTDLRFGIVSEKYEKGITSFDYERIKAEEELDRNIAAYVTFAADIFAKSVIKSDYR |
| SEQ ID NO: | TKKDNNSDVLQYSDKEFRNSEVIRDNAEKNILQYWGGYSRWGTENSKLPENNKVSDFGKLDVSDLCI |
| 4217 | DIKKHLAGIRNSSVHYTTKIKNESAADGSNVKILFEKDLADINHYADKYYSNNVWMFYSLEDINKLIAF |
| | LYKEKRVIRQVQIPSFSRILKRKAMQDVINEIFKDDFDENIVNPELKEKYRNSLYFMLKEIYYNAFIIQPE |
| | LKEKFKDKIKTMKSELYNKLKTIDKKEYKALYCMLSNEESALKNFADRIYSVDGDNVSFADICKILMT |
| | DYNMQNQEKKNIESMEQKKKNRNSRQIVRILKLIHFRMKWNQMQIQTDLWMRQQKHPQVQRNRMR |
| | MDLHMNILKSLIHTA |
| OWCB01.1 | MKVSKVKHRRTAVSVNKKNNTVKGILYDDPIKKDSKGDGASAYVSTKYVVDDVVRNSSRLYSPFNSK |
| SEQ ID NO: | KLIIDDKTKVVANSLRQHFKNFVKIYLNCESIDEQQMKFTPDNKYLMDNRVRISLPSDVNEEKLVEAIV |
| 4218 | NSSLRKSLNKKCNIQIKAGLRETFDIPELIKKAIKIYCIDEKRNLNDAEKLDMYALFSFMYEDKYKNRQ |
| | KKLIINSISNQVTKVKVCENGNRLLKLSIADTKKKPLWDFMIEYSNSDKKKQDTMLRNIRKSIVLFVCG |
| | VENYKNIENDNKLDICSWDGYDINENQQFVCVNTNNSNDDYFISSTELRRANLDHYMKAVAKLNDDR |
| | NKFWFQHFESVIETFFSKKAKRNIERIKSAYLCEYLWRDFCSYVALKYVDLGKGVYHFTMADKLALIN |
| | RNKYEKSIIFGEIESRYNNGISSFDYERIKAEELFERNISTYTTFATNIFSKAVVQDDYIKNHNKASDVLQ |
| | YSDKEFSDSKVLRNDAMKRILQYWGGQSRWNNTLNKINVDTLCIDIKEHLSNIRNSSVHYTSKVSLSG |
| | DKNESIVYMLFKKDFAEIRNIFASKYYSNNVWMFYSIEKINGLMEYLYGDNSTVIDAQIPAYNNIIKRK |
| | NIADVIEKIIKKNSYKTINELELIKKYRACLYFILKEIYYNRFIKQENLKEQFIQFVDNDNNLLDDNKNTF |
| | YIQKRHLRSPNNHK |
| ORVG01.1 | MTEYNQQNNQIKKVRSSNDSIFDQPIYQHYKVLLKKAIANAFADYLKNNKDLFGFIGKPFKANEIREID |
| SEQ ID NO: | KEQFLPDWTSRKYEALCIEVSGSQELQKWYIVGKFLNAMSLNLMVGSMRSYIQYVTDIKRRAASIGNE |
| 4219 | LHVSVQDVEKVEKWVQVIEVCSLLASRTSNQFEDYFNDKDDYARYLKSYVDFSNVDMPSEYSALVDF |
| | SNEEQSDLYVDPKNPKVNRNIVHSKLFAADHILRDIVEPVSKDNIEEFYSQKAEIAYCKIKGKEITAEEQ |
| | KAVLKYQKLKNRVELRDIVEYGEIINELLGQLINWSFMRERDLLYFQLGFHYDCLRNDSKKPERYKNI |
| | KVDENSIKDAILYQIIGMYVNGVTVYAPEKDDDKLKEQCVKGGVGVKVSAFHRYSKYLGLNEKTLYN |
| | AGLEIFEVVAEHEDIINLRNGIDHFKYYLGDYRSMLSIYSEVFDRFFTYDIKYQKNVLNLLQNILLRHNV |
| | IVEPILESGFKTIGEQTKPGAKLSIRSIKSDTFQYKVKGGTLITDAKDERYLETIRKILYYAENEEDNLKKS |
| | VVVTNADKYEKNKESDDQNKQKEKKNKDNKGKKNEETKSDAEKNNNERLSYNPFANLNFKLSN |
| ULZH01.1_2 | VCSLLASRTSNQFEDYFNDKDDYARYLKSYVDFSNVDMPSEYSALVDFSNEEQSDLYVDPKNPKVNR |
| SEQ ID NO: | NIVHSKLFAADHILRDIVEPVSKDNIEEFYSQKAEIAYCKIKGKEITAEEQKAVLKYQKLKNRVELRDIV |
| 4220 | EYGEIINELLGQLINWSFMRERDLLYFQLGFHYDCLRNDSKKPERYKNIKVDENSIKDAILYQIIGMYVN |
| | GVTVYAPEKDDDKLKEQCVKGGVGVKVSAFHRYSKYLGLNEKTLYNAGLEIFEVVAEHEDIINLRNGI |
| | DHFKYYLGDYRSMLSIYSEVFDRFFTYDIKYQKNVLNLLQNILLRHNVIVEPILESGFKTIGEQTKPGAK |
| | LSIRSIKSDTFQYKVKGGILITDAKDERYLETIRKILYYAENEEDNLKKSVVVTNADKYEKNKESDDQN |
| | KQKEKKNKDNKGKKNEETKSDAEKNNNERLSYNPFANLNFKLSN |
| IMG_ | VSAVKEISTGNSNGNVIGAAVKNNSGKMGIIDSNGQNKVEQAYDSKKPEGYKNIKVDENSIKDAILYQI |
| 3300014770_2 | IGMYVNGVTVYAPEKDGDKLKEQCVKGGVGVKVSAFHRYSKYLGLNEKTLYNAGLEIFEVVAEHEDI |
| SEQ ID NO: | INLRNGIDHFKYYLGDYRSMLSIYSEVFDRFFTYDIKYQKNVLNLLQNILLRHNVIVEPILESGFKTIGEQ |
| 4221 | TKPGAKLSIRSIKSDTFQYKVKGGILITDAKDERYLETIRKILYYAENEEDNLKKSVVVTNADKYEKNK |
| | ESDDQNKQKEKKNKDNKGKKNEEIKSDAEKNNNERLSYNPFANLNFKLSN |
| OQVO01.1 | LSLGIKEERICETKEDNWWPNLEMPGLCGPDSEMFYFRSDDEIPEKFDPDDNRWVEIWNDVFMQYNH |
| SEQ ID NO: | KEDGTIEILKHKNVDTGMGLERVTAILEGVNDNYLSSIWKDVIEKICEISNTKYEDNKESIRIIADHIRTS |
| 4222 | VFISADYSGIKPSNVGQGYILRRLIRRSIRHAKKLNIDISSNWDIEIAKLIINKYKKYYKELEENENVVYE |
| | VLTNEKNKFNKTIEKGLREFEKVTKDNNDIDASTAFKLYDTYGFPLELTVELAHEKNIKVDENSIKDAI |
| | LYQIIGMYVNGVTVYAPEKDGDKLKEQCVKGGVGVKVSAFHRYSKYLGLNEKTLYNAGLEIFEVVAE |
| | HEDIINLRNGIDHFKYYLGDYRSMLSIYSEVFDRFFTYDIKYQKNVLNLLQNILLRHNVIVEPILESGFKT |
| | IGEQTKPGAKLSIRSIKSDTFQYKVKGGILITDAKDERYLETIRKILYYAENEEDNLKKSVVVTNADKYE |
| | KNKESDDQNKQKEKKNKDNKGKKNEEIKSDAEKNNNERLSYNPFANLNFKLSN |
| OQCX01.1_ | LIKSSFCLNGHQKNTYHYARKLEKAQNSKKWYIVGKFLNSRSLNLMAGSMRSYIQYVNDIKRRADGIG |
| 2 | NELHVIAQNLDVVDKWVQVIEVCLLLSSRVSNEFEDYFYDKDDYAAYLKSYVDFDNSDMPSEYSALV |
| SEQ ID NO: | EFSDQGKVDLYVDPSNPKVNRNIVQSKLFAADYILRDIIEPVSKDEIEDFYNQKDEITTCKIKGAELTDE |
| 4223 | EQKKILKYQKLKNRVELRDVVEYGEIINELLGQLINWSFMRERDLLYFQLGFHYNCLRNDSAKPEEYK |
| | NLVLDDVSIKDAILHQIIGMYVNGVAIYAPGKDKNKLESQCVKGRVGGKIGAFCGYSLYLKLAADTLY |
| | NAGLEVFEVLPEHEDIINLRNGIDHFKFYLGGYRSIISLYSEVFDRFFTYDMKYQKNVLNLLQNILLRHN |
| | VIIEPIFESGIKKIGKDTKPCAKLCISSIKSDSFEYKIKDGTLITDAKDKRYLETIKKLLYYPDIESNVKILLR |
| | KDNFNQNKDKKNVNNRKTKNN |
| OPHK01.1 | MLDHLYNNKVSRAAQVPSYNSVMVRKYFPENITSTLKYQKPGYDEDTLEKWYSACYYLLKEIYYNSF |
| SEQ ID NO: | LQSDEALALFEESVNNLKGDNKDQELAVKNFRNNYKNIKSSCTSFSQVCQMYMTEYNQQNNQFKKV |
| 4224 | RSSKDSIVDKPIYQHYKLLLKKVIANAFASYLQHNEELFGFIGKPLKVNCLKEIDKEQFLPEWTSKKYVS |
| | LCEEVRKSPELQKWYIVGKFLNSRSLNLMAGSMRSYIQYVNDIKRRADGIGNELHVIAQNLDVVNKW |
| | VQVIEVCLLLSSRVSNEFEDYFYDKDDYAAYLKSYVDFDNSDMPSEYSALVEFSDQGKVDLYVDPSNP |
| | KVNRNIVQSKLFAADYILRDIIEPVSKDEIEDFYNQKDEITTCKIKGAELTDEEQKKILKYQKLKNRVEL |
| | RDVVEYGEIINELLGQLINWSFMRERDLLYFQLGFHYNCLRNDSAKPEEYKNLVLDDISIKDAILHQIIG |
| | VYVNGVAIYAPGKDKNKLESQCVKGRVGGKIGAFCGYSLYLKLAADTLYNAGLEVFEVLPEHEDIINL |
| | RNGIDHFKFYLGGYRSIISLYSEVFDRFFTYDMKYQKNVLNLLQNILLRHNVIIEPIFESGIKKIGKDTKP |
| | CAKLSIRSIISDSFEYKIKDGNLIADAKDKRYLETIKKILFYPEVEPEVRILSSKDSFEQNNQYGYMKEKS |
| | ENNKNKKNKKNNGNRDEKKNSDGLTYNPFLNLPFELPE |
| UXRR01.1 | MIKAQEALQRELAVNVAFAANNLARAVCDMTNLKDKESDFLIWNKKDIANKLKNKDDMASVSVVLQ |
| SEQ ID NO: | FFGGKSSWDIDAFREAYKGNKYNYEVCFIDDLRKAVYAARNESFHFKTALVNNDIWNTEFFGKLFIKE |
| 4225 | TEICLDIEKDRFYSNNLPVFYSDNDLKKMLDHLYNNKVSRAAQVPSYNSVMVRKYFPENITSTLKYQK |
| | PGYDEDTLEKWYSACYYLLKEIYYNSFLQSDEALALFEESVNNLKGDNKDQELAVKNFRNNYKNIKSS |
| | CTSFSQVCQMYMTEYNQQNNQFKKVRSSKDSIVDKPIYQHYKLLLKKVIANAFASYLQHNEELFGFIG |
| | KPLKVNCLKEIDKEQFLPEWTSKKYVSLCEEVRKSPELQKWYIVGKFLNSRSLNLMAGSMRSYIQYVN |
| | DIKRRADGIGNELHVIAQNLDVVNKWVQVIEVCLLLSSRVSNEFEDYFYDKDDYAAYLKSYVDFDNS |
| | DMPSEYSALVEFSDQGKVDLYVDPSNPKVNRNIVQSKLFAADYILRDIIEPVSKDEIEDFYNQKDEITTC |
| | KIKGAELTDEEQKKILKYQKLKNRVELRDVVEYGEIINELLGQLINWSFMRERDLLYFQLGFHYNCLR |
| | NDSAKPEEYKNLVLDDISIKDAILHQIIGVYVNGVAIYAPGKDKNKLESQCVKGRVGGKIGAFCGYSLY |
| | LKLAADTLYNAGLEVFEVLPEHEDIINLRNGIDHFKFYLGGYRSIISLYSEVFDRFFTYDMKYQKNVLN |
| | LLQNILLRHNVIIEPIFESGIKKIGKDTKPCAKLSIRSIISDSFEYKIKDGNLIADAKDKRYLETIKKILFYPE |
| | VEPEVRILSSKDSFEQNNQYGYMKEKSENNKNKKNKKNNGNRDEKKNSDGLTYNPFLNLPFELPE |
| UZJD01.1 | MVRKYFPENITSTLKYQKPGYDEDTLEKWYSACYYLLKEIYYNSFLQSDEALALFEESVNNLKGDNKD |
| SEQ ID NO: | QELAVKNFRNNYKNIKSSCTSFSQVCQMYMTEYNQQNNQFKKVRSSKDSIVDKPIYQHYKLLLKKVIA |
| 4226 | NAFASYLQHNKELFGFIGKPLKVNCLKEIDKEQFLPEWTAKKYVSLCEEVRKSPELQKWYIVGKFLNS |
| | RSLNLMAGSMRSYIQYVNDIKRRADGIGNELHVIAQNLDVVDKWVQVIEVCLLLSSRVSNEFEDYFYD |
| | KDDYAAYLKSYVDFDNSDMPSEYSALVEFSDQGKVDLYVDPSNPKVNRNIVQSKLFAADYILRDIIEP |
| | VSKDEIEDFYNQKDEITICKIKGAELTDEEQKKILKYQKLKNRVELRDVVEYGEIINELLGQLINWSFMR |
| | ERDLLYFQLGFHYNCLRNDSAKPEEYKNLVLDDISIKDAILHQIIGMYVNGVAIYAPGKDENKLESQCA |
| | QGGAGGKIGAFCRYSLYLKLAADTLYNAGLEVFEVLPEHEDIIKLRNGIDHFKFYLGGYRSIMSLYSEV |
| | FDRFFTYDMKYQKNVLNLLQNILLRHNVIIEPIFEFGIKKIGKDTKPCAKLCISSIKSDSFEYKIKDGTLIT |
| | DAKDKRYLETIKKILFYPEVESEVRILSSKDSFEQNNQYGYMKGKSENNKNKKNKKNNGNRDEKKNS |
| | DGLTYNPFLNLPFELPE |
| OGYB01.1 | LQHNKELFGFIGKPLKVNCLKEIDKEQFLPEWTAKKYVSLCEEVRKSPELQKWYIVGKFLNSRSLNLM |
| SEQ ID NO: | AGSMRSYIQYVNDIKRRADGIGNELHVIAQNLDVVDKWVQVIEVCLLLSSRVSNEFEDYFYDKDDYA |
| 4227 | AYLKSYVDFDNSDMPSEYSALVEFSDQGKVDLYVDPSNPKVNRNIVQSKLFAADYILRDIIEPVSKDEI |
| | EDFYNQKDEITICKIKGAELTDEEQKKILKYQKLKNRVELRDVVEYGEIINELLGQLINWSFMRERDLL |
| | YFQLGFHYNCLRNDSAKPEEYKNLVLDDISIKDAILHQIIGMYVNGVAIYAPGKDKNKLESQCVKGRV |
| | GGKIGAFCGYSLYLKLAADTLYNAGLEVFEVLPEHEDIINLRNGIDHFKFYLGGYRSIISLYSEIFDRFFT |
| | YDMKYQKNVLNLLQNILLRHNVIIEPIFESGIKKIGKDTKLCAKLCISSLKSDSFEYKIKDGTLITDAKDK |
| | RYLETUCKILFYPEVESEVRILSSKDSFEQNNQYGYMKGKSENNKNKKNKKNNGNRDEKKNSDGLTYN |
| | PFLDLPFELPE |
| OYDY01.1 | MGKLVMNVLVKSSNGITQVSADAKLLSQRKVFIEGEISPETACEFIKKIIVLNAENQEKFIDVLINSPGGE |
| SEQ ID NO: | INSGLAMYDVIQSSKAPIRVFCIGRAYSMAKYLGLNEKTLYNAGLEIFEVVAEHEDIINLRNGIDHFKYY |
| 4228 | LGDYRSMLSIYSEVFDRFFTYDIKYQKNVLNLLQNILLRHNVIVEPILESGFKTIGEQTKPGAKLSIRSIKS |
| | DTFQYKVKGGTLITDAKDERYLETIRKILYYAENEEDNLKKSVVVTNADKYEKNKESDDQNKQKEKK |
| | NKDNKGKKNEETKSDAEKNNNERLSYNPFANLNFKLSN |
| ORVG01.1_ | MKLSKEKHTRSAVANEEQNIGGILYRFPGKSIDGVKDQMLRRDKEVKKLYNVFNQIQVGTKPKKWNN |
| 2 | DEKLSPEENERRAQQKNIKIENYKWREACSKYVKSSQKTINYVIFYSYGKAENKLRYMRKNEDILKKM |
| SEQ ID NO: | QEEEKLPKFSGGKLEDFVAYTLRKSLVVSKYDTQEFDSVAAMVVFLECIGKSNISDHEKEIVCKLLELIR |
| 4229 | KDFSKLDPNVKGSQGANIVRSVRNQNMIVQPQGDRFLFPQVYAKENETVTNKNVEKEGLNEFLLNYA |
| | NLDDEKRAESLRKLRRILDVYFSAPNYYEKDMDITLSDNIEKGKFNVWEKHECGKKVTDLFVDIPDVL |
| | MEAGAENIKLDAVVEKRERKVLNDRVRKQNIICYRYTRAVVEKYNSNEPLFFENNAINQYWIHHIENA |
| | VERILKNCKAGKLFKLRKGYLAEKVWKDAINLISIKYIALGKAVYNFALDDIWKDKKNKELGIVDERIR |
| | NGITSFDYEMIKAHENLQRELAVDIAFSVNNLARAVCDMSNLGNKESDFLLWKRNDIADKLKNKDDM |
| | ASVSAVLQFFGGKSSWDINIFKEAYKGKKKYNYEVRFIDDLRKAIYCARNENFHFKTALVNDEKWNTE |
| | LFGKIFKRETEFCLNVEKDRFYSNNLYMFYQVSELRNMLDHLYSRSVSRAAQVPSYNSVIVRTAFPEYI |
| | TNVLGYQKPGYDADTLGKWYSACYYLLKEIYYNSFLQSDRALQLFEKSVKTLSWDDEEQKRAVDNF |
| | KKYFSDIKSACTSLAQVCQIYILDSRKRDEDTTSRIAAN |
| OHCP01.1 | MKLSKEKHTRSAVANEEQNIGGILYRFPGKSIDGVKDQMLRRDKEVKKLYNVFNQIQVGTKPKKWNN |
| SEQ ID NO: | DEKLSPEENERRAQQKNIKMKNYKWREACSKYVESSQRIINDVIFYSYRKAKNKLRYMRKNEDILKK |
| 4230 | MQEAEKLSKFSGGKLEDFVAYTLRKSLVVSKYDTQEFDSVAAMVVFLECIGKNNISDHEREIVCKLLE |
| | LIRKDFSKLDPNVKGSQGANIVRSVRNQNMIVQPQGDRILFPQVYAKENETVTNKNVEKEGLNEFLLN |
| | YANLDDEKRAESLRKLRRILDVYFSAPNHYEKDMDITLSDNIEKEKFNVWEKHECGKKETGLFVDIPD |
| | VLMEAEAENIKLDAVVEKRERKVLNDRVRKQNIICYRYTRAVVEKYNSNEPLFFENNAINQYWIHHIE |
| | NAVERILKNCKAGKLFKLRKGYLAEKVWKDAINLISIKYIALGKAVYNFALDDIWKDKKNKELGIVDE |
| | RIRNGITSFDYEMIKAHENLQRELAVDIAFSVNNLARAVCDMSNLGNKESDFLLWKRNDIADKLKNKD |
| | DMASVSAVLQFFGGKSSWDINIFKDAYKGKKKYNYEVRFIDDLRKAIYCARNENFHFKTALVNDEKW |
| | NTELFGKIFERETEFCLNVEKDRFYSNNLYMFYQVSELRNMLDHLYSRSVSRAAQVPSYNSVIVRTAFP |
| | EYITNVLGYQKPSYDADTLGKWYSACYYLLK |
| OPVG01.1 | MKLSKEKHTRSAVANEEQNIGGILYRFPGKSIDGVKDQMLRRDKEVKKLYNVFNQIQVGTKPKKWNN |
| SEQ ID NO: | DEKLSPEENERRAQQKNIKMKNYKWREACSKYVESSQRIINDVIFYSYRKAKNKLRYMRKNEDILKK |
| 4231 | MQEAEKLSKFSGGKLEDFVAYTLRKSLVVSKYDTQEFDSVAAMVVFLECIGKNNISDHEREIVCKLLE |
| | LIRKDFSKLDPNVKGSQGANIVRSVRNQNMIVQPQGDRFLFPQVYAKENETVTNKNVEKEGLNEFLLN |
| | YANLDDEKRAESLRKLRRILDVYFSAPNHYEKDMDITLSDNIEKEKFNVWEKHECGKKETGLFVDIPD |
| | VLMEAEAENIKLDAVVEKRERKVLNDRVRKQNIICYRYTRAVVEKYNSNEPLFFENNAINQYWIHHIE |
| | NAVERILKNCKAGKLFKLRKGYLAEKVWKDAINLISIKYIALGKAVYNFALDDIWKDKKNKELGIVDE |
| | RIRNGITSFDYEMIKAHENLQRELAVDIAFSVNNLARAVCDMSNLGNKESDFLLWKRNDIADKLKNKD |
| | DMASVSAVLQFFGGKSSWDINIFKDAYKGKKKYNYEVRFIDDLRKAIYCARNENFHFKTALVNDEKW |
| | NTELFGKIFERETEFCLNVEKDRFYSNNLYMFYQVSELRNMLDHLYSRSVSRAAQVPSYNSVIVRTAFP |
| | EYITNVLGYQKPSYDADTLGKWYSACYYLLKEIYYNSFLQSDRALQLFEKSVKTLSWDDKKQQRAVD |
| | NFKDHFSDIKSACTSLAQVCQIYMTEYNQQNNQIKKVRSSNDSIFDQPVYQHYKVLLKKAIANAFADY |
| | LKNNKDLFGFIGKPFKANEIREIDKEQFLPDWTSRKYEALCIEVSG |
| OHRU01.1 | MKLSKEKHTRSAVANEEQNIGGILYRFPGKSIDGVKDQMLRRDKEVKKLYNVFNQIQVGTKPKKWNN |
| SEQ ID NO: | DEKLSPEENERRAQQKNIKMKNYKWREACSKYVESSQRIINDVIFYSYRKAKNKLRYMRKNEDILKK |
| 4232 | MQEAEKLSKFSGGKLEDFVAYTLRKSLVVSKYDTQEFDSVAAMVVFLECIGKNNISDHEREIVCKLLE |
| | LIRKDFSKLDPNVKGSQGANIVRSVRNQNMIVQPQGDRFLFPQVYAKENETVTNKNVEKEGLNEFLLN |
| | YANLDDEKRAESLRKLRRILDVYFSAPNHYEKDMDITLSDNIEKEKFNVWEKHECGKKETGLFVDIPD |
| | VLMEAEAENIKLDAVVEKRERKVLNDRVRKQNIICYRYTRAVVEKYNSNEPLFFENNAINQYWIHHIE |
| | NAVERILKNCKAGKLFKLRKGYLAEKVWKDAINLISIKYIALGKAVYNFALDDIWKDKKNKELGIVDE |
| | RIRNGITSFDYEMIKAHENLQRELAVDIAFSVNNLARAVCDMSNLGNKESDFLLWKRNDIADKLKNKD |
| | DMASVSAVLQFFGGKSSWDINIFKEAYKGKKKYNYEVRFIDDLRKAIYCARNENFHFKTALVNDEKW |
| | NTELFGKIFKRETEFCLNVEKDRFYSNNLYMFYQVSELRNMLDHLYSRSVSRAAQVPSYNSVIVRTAFP |
| | EYITNVLGYQKPGYDADTLGKWYSACYYLLKEIYYNSFLQS |
| OHIL01.1 | MKLSKEKHTRSAVANEEQNIGGILYRFPGKSIDGVKDQMLRRDKEVKKLYNVFNQIQVGTKPKKWNN |
| SEQ ID NO: | DEKLSPEENERRAQQKNIKMKNYKWREACSKYVESSQRIINDVIFYSYRKAKNKLRYMRKNEDILKK |
| 4233 | MQEAEKLSKFSGGKLEDFVAYTLRKSLVVSKYDTQEFDSVAAMVVFLECIGKNNISDHEREIVCKLLE |
| | LIRKDFSKLDPNVKGSQGANIVRSVRNQNMIVQPQGDRFLFPQVYAKENETVTNKNVEKEGLNEFLLN |
| | YANLDDEKRAESLRKLRRILDVYFSAPNHYEKDMDITLSDNIEKEKFNVWEKHECGKKETGLFVDIPD |
| | VLMEAEAENIKLDAVVEKRERKVLNDRVRKQNIICYRYTRAVVEKYNSNEPLFFENNAINQYWIHHIE |
| | NAVERILKNCKAGKLFKLRKGYLAEKVWKDAINLISIKYIALGKAVYNFALDDIWKDKKNKELGIVDE |
| | RIRNGITSFDYEMIKAHENLQRELAVDIAFSVNNLARAVCDMSNLGNKESDFLLWKRNDIADKLKNKD |
| | DMASVSAVLQFFGGKSSWDINIFKEAYKGKKKYNYEVRFIDDLRKAIYCARNENFHFKTALVNDEKW |
| | NTELFGKIFKRETEFCLNVEKDRFYSNNLYMFYQVSELRNMLDHLYSRSVSRAAQVPSYNSVIVRTAFP |
| | EYITNVLGYQKPGYDADTLGKWYSACY |
| OKSW01.1 | MKLSKEKHTRSAVANEEQNIGGILYRFPGKSIDGVKDQMLRRDKEVKKLYNVFNQIQVGTKPKKWNN |
| SEQ ID NO: | DEKLSPEENERRAQQKNIKMKNYKWREACSKYVESSQRIINDVIFYSYRKAKNKLRYMRKNEDILKK |
| 4234 | MQEAEKLSKFSGGKLEDFVAYTLRKSLVVSKYDTQEFDSVAAMVVFLECIGKNNISDHEREIVCKLLE |
| | LIRKDFSKLDPNVKGSQGANIVRSVRNQNMIVQPQGDRFLFPQVYAKENETVTNKNVEKEGLNEFLLN |
| | YANLDDEKRAESLRKLRRILDVYFSAPNHYEKDMDITLSDNIEKEKFNVWEKHECGKKETGLFVDIPD |
| | VLMEAEAENIKLDAVVEKRERKVLNDRVRKQNIICYRYTRAVVEKYNSNEPLFFENNAINQYWIHHIE |
| | NAVERILKNCKAGKLFKLRKGYLAEKVWKDAINLISIKYIALGKAVYNFALDDIWKDKKNKELGIVDE |
| | RIRNGITSFDYEMIKAHENLQRELAVDIAFSVNNLARAVCDMSNLGNKESDFLLWKRNDIADKLKNKD |
| | DMASVSAVLQFFGGKSSWDINIFKEAYKGKKKYNYEVRFIDDLRKAIYCARNENFHFKTALVNDEKW |
| | NTELFGKIFKRETEFCLNVEKDRFYSNNLYMFYQVSELRNMLDHLYSRSVSRAAQVPSYNSVIVRTAFP |
| | EYITNVLGYQKPGYDADTLGKWYSACYYLLKEI |
| OHSM01.1 | MKLSKEKHTRSAVANNGDIKSAEVNNGNTKSEEVNNGDTRSAVANEEQNIGGILYRFPGKSIDGVKDQ |
| SEQ ID NO: | MLRRDKEVKKLYNVFNQIQVGTKPKKWNNDEKLSPEENERRAQQKNIKMKNYKWREACSKYVESSQ |
| 4235 | RIINDVIFYSYRKAKNKLRYMRKNEDILKKMQEAEKLSKFSGGKLEDFVAYTLRKSLVVSKYDTQEFD |
| | SVAAMVVFLECIGKNNISDHEREIVCKLLELIRKDFSKLDPNVKGSQGANIVRSVRNQNMIVQPQGDRI |
| | LFPQVYAKENETVTNKNVEKEGLNEFLLNYANLDDEKRAESLRKLRRILDVYFSAPNHYEKDMDITLS |
| | DNIEKEKFNVWEKHECGKKETGLFVDIPDVLMEAEAENIKLDAVVEKRERKVLNDRVRKQNIICYRYT |
| | RAVVEKYNSNEPLFFENNAINQYWIHHIENAVERILKNCKAGKLFKLRKGYLAEKVWKDAINLISIKYI |
| | ALGKAVYNFALDDIWKDKKNKELGIVDERIRNGITSFDYEMIKAHENLQRELAVDIAFSVNNLARAVC |
| | DMSNLGNKESDFLLWKRNDIADKLKNKDDMASVSAVLQFFGGKSSWDINIFKDAYKGKKKYNYEVR |
| | FIDDLRKAIYCARNENFHFKTALVNDEKWNTELFGKIFERETEFCLNVEKDRFYSNNLYMFYQVSELR |
| | NMLDHLYSRSVSRAAQVPSYNSVIVRTAFPEYITNVLGYQ |
| OZCB01.1 | MKLSKEKHTRSAVANNGDIKSAEVNNGNTKSEEVNNGDTRSAVANEEQNIGGILYRFPGKSIDGVKDQ |
| SEQ ID NO: | MLRRDKEVKKLYNVFNQIQVGTKPKKWNNDEKLSPEENERRAQQKNIKMKNYKWREACSKYVESSQ |
| 4236 | RIINDVIFYSYRKAKNKLRYMRKNEDILKKMQEAEKLSKFSGGKLEDFVAYTLRKSLVVSKYDTQEFD |
| | SVAAMVVFLECIGKNNISDHEREIVCKLLELIRKDFSKLDPNVKGSQGANIVRSVRNQNMIVQPQGDRF |
| | LFPQVYAKENETVTNKNVEKEGLNEFLLNYANLDDEKRAESLRKLRRILDVYFSAPNHYEKDMDITLS |
| | DNIEKEKFNVWEKHECGKKETGLFVDIPDVLMEAEAENIKLDAVVEKRERKVLNDRVRKQNIICYRYT |
| | RAVVEKYNSNEPLFFENNAINQYWIHHIENAVERILKNCKAGKLFKLRKGYLAEKVWKDAINLISIKYI |
| | ALGKAVYNFALDDIWKDKKNKELGIVDERIRNGITSFDYEMIKAHENLQRELAVDIAFSVNNLARAVC |
| | DMSNLGNKESDFLLWKRNDIADKLKNKDDMASVSAVLQFFGGKSSWDINIFKDAYKGKKKYNYEVR |
| | FIDDLRKAIYCARNENFHFKTALVNDEKWNTELFGKIFERETEFCLNVEKDRFYSNNLYMFYQVSELR |
| | NMLDHLYSRSVSRAAQVPSYNSVIVRTAFPEYITNVLGYQKPGYDADTLGKWYSACSVSYTHLTLPTI |
| | A |
| CDYI01.1 | MKLSKEKHTRSAVANNGDIKSAEVNNGNTKSEEVNNGDTRSAVANEEQNIGGILYRFPGKSIDGVKDQ |
| SEQ ID NO: | MLRRDKEVKKLYNVFNQIQVGTKPKKWNNDEKLSPEENERRAQQKNIKMKNYKWREACSKYVESSQ |
| 4237 | RIINDVIFYSYRKAKNKLRYMRKNEDILKKMQEAEKLSKFSGGKLEDFVAYTLRKSLVVSKYDTQEFD |
| | SVAAMVVFLECIGKNNISDHEREIVCKLLELIRKDFSKLDPNVKGSQGANIVRSVRNQNMIVQPQGDRF |
| | LFPQVYAKENETVTNKNVEKEGLNEFLLNYANLDDEKRAESLRKLRRILDVYFSAPNHYEKDMDITLS |
| | DNIEKEKFNVWEKHECGKKETGLFVDIPDVLMEAEAENIKLDAVVEKRERKVLNDRVRKQNIICYRYT |
| | RAVVEKYNSNEPLFFENNAINQYWIHHIENAVERILKNCKAGKLFKLRKGYLAEKVWKDAINLISIKYI |
| | ALGKAVYNFALDDIWKDKKNKELGIVDERIRNGITSFDYEMIKAHENLQRELAVDIAFSVNNLARAVC |
| | DMSNLGNKESDFLLWKRNDIADKLKNKDDMASVSAVLQFFGGKSSWDINIFKDAYKGKKKYNYEVR |
| | FIDDLRKAIYCARNENFHFKTALVNDEKWNTELFGKIFERETEFCLNVEKDRFYSNNLYMFYQVSELR |
| | NMLDHLYSRSVSRAAQVPSYNSVIVRTAFPEYITNVLGYQKPGYDADTLGKWYSACYYLLKEIYYNSF |
| | LQSD |
| OZYB01.1 | MKLSKEKHTRSAVANNGDIKSAEVNNGNTKSEEVNNGDTRSAVANEEQNIGGILYRFPGKSIDGVKDQ |
| SEQ ID NO: | MLRRDKEVKKLYNVFNQIQVGTKPKKWNNDEKLSPEENERRAQQKNIKMKNYKWREACSKYVESSQ |
| 4238 | RIINDVIFYSYRKAKNKLRYMRKNEDILKKMQEAEKLSKFSGGKLEDFVAYTLRKSLVVSKYDTQEFD |
| | SVAAMVVFLECIGKNNISDHEREIVCKLLELIRKDFSKLDPNVKGSQGANIVRSVRNQNMIVQPQGDRF |
| | LFPQVYAKENETVTNKNVEKEGLNEFLLNYANLDDEKRAESLRKLRRILDVYFSAPNHYEKDMDITLS |
| | DNIEKEKFNVWEKHECGKKETGLFVDIPDVLMEAEAENIKLDAVVEKRERKVLNDRVRKQNIICYRYT |
| | RAVVEKYNSNEPLFFENNAINQYWIHHIENAVERILKNCKAGKLFKLRKGYLAEKVWKDAINLISIKYI |
| | ALGKAVYNFALDDIWKDKKNKELGIVDERIRNGITSFDYEMIKAHENLQRELAVDIAFSVNNLARAVC |
| | DMSNLGNKESDFLLWKRNDIADKLKNKDDMASVSAVLQFFGGKSSWDINIFKDAYKGKKKYNYEVR |
| | FIDDLRKAIYCARNENFHFKTALVNDEKWNTELFGKIFERETEFCLNVEKDRFYSNNLYMFYQVSELR |
| | NMLDHLYSRSVSRAAQVPSYNSVIVRTAFPEYITNVLGYQKPGYDADTLGKWYSACYYLLKEIYYNSF |
| | LQSDRACLLYTSPSPRDGLLSR |
| OIPQ01.1 | MKLSKEKHTRSAVANNGDIKSAEVNNGNTKSEEVNNGDTRSAVANEEQNIGGILYRFPGKSIDGVKDQ |
| SEQ ID NO: | MLRRDKEVKKLYNVFNQIQVGTKPKKWNNDEKLSPEENERRAQQKNIKMKNYKWREACSKYVESSQ |
| 4239 | RIINDVIFYSYRKAKNKLRYMRKNEDILKKMQEAEKLSKFSGGKLEDFVAYTLRKSLVVSKYDTQEFD |
| | SVAAMVVFLECIGKNNISDHEREIVCKLLELIRKDFSKLDPNVKGSQGANIVRSVRNQNMIVQPQGDRF |
| | LFPQVYAKENETVTNKNVEKEGLNEFLLNYANLDDEKRAESLRKLRRILDVYFSAPNHYEKDMDITLS |
| | DNIEKEKFNVWEKHECGKKETGLFVDIPDVLMEAEAENIKLDAVVEKRERKVLNDRVRKQNIICYRYT |
| | RAVVEKYNSNEPLFFENNAINQYWIHHIENAVERILKNCKAGKLFKLRKGYLAEKVWKDAINLISIKYI |
| | ALGKAVYNFALDDIWKDKKNKELGIVDERIRNGITSFDYEMIKAHENLQRELAVDIAFSVNNLARAVC |
| | DMSNLGNKESDFLLWKRNDIADKLKNKDDMASVSAVLQFFGGKSSWDINIFKDAYKGKKKYNYEVR |
| | FIDDLRKAIYCARNENFHFKTALVNDEKWNTELFGKIFERETEFCLNVEKDRFYSNNLYMFYQVSELR |
| | NMLDHLYSRSVSRAAQVPSYNSVIVRTAFPEYITNVLGYQKPSYDADTLGKWYSACYYLLKEIYYNSF |
| | LQSDRALQLFEKSVKTLSWDDKKQQ |
| UPNA01.1 | MKLSKEKHTRSAVANNGDIKSAEVNNGNTKSEEVNNGDIRSAVANEEQNIGGILYRFPGKSIDGVKDQ |
| SEQ ID NO: | MLRRDKEVKKLYNVFNQIQVGTKPKKWNNDEKLSPEENERRAQQKNIKMKNYKWREACSKYVESSQ |
| 4240 | RIINDVIFYSYRKAENKLRYMRKNEDILKKMQEAEKLSKFSGGKLEDFVAYTLRKSLVVSKYDTQEFD |
| | SVAAMVVFLECIGKNNISDHEREIVCKLLELIRKDFSKLDPNVKGSQGANIVRSVRNQNMIVQPQGDRF |
| | LFPQVYAKENETVTNKNVEKEGLNEFLLNYANLDDEKRAESLRKLRRILDVYFSAPNHYEKDMDITLS |
| | DNIEKEKFNVWEKHECGKKETGLFVDIPDVLMEAEAENIKLDAVVEKRERKVLNDRVRKQNIICYRYT |
| | RAVVEKYNSNEPLFFENNAINQYWIHHIENAVERILKNCKAGKLFKLRKGYLAEKVWKDAINLISIKYI |
| | ALGKAVYNFALDDIWKDKKNKELGIVDERIRNGITSFDYEMIKAHENLQRELAVDIAFSVNNLARAVC |
| | DMSNLGNKESDFLLWKRNDIADKLKNKDDMASVSAVLQFFGGKSSWDINIFKEAYKGKKKYNYEVR |
| | FIDDLRKAIYCARNENFHFKTALVNDEKWNTELFGKIFERETEFCLNVEKDRFYSNNLYMFYQVSELR |
| | NMLDHLYSRSVSRAAQVPSYNSVIVRTAFPEYITNVLGYQKPGYDADTLG |
| OPAV01.1 | MKLSKEKHTRSAVANNGDIKSAEVNNGNTKSEEVNNGDIRSAVANEEQNIGGILYRFPGKSIDGVKDQ |
| SEQ ID NO: | MLRRDKEVKKLYNVFNQIQVGTKLKKWNNDEKLSPEENERRAQQKNIKMKNYKWREACSKYVESSQ |
| 4241 | RIINDVIFYSYRKAKNKLRYMRKNEDILKKMQEAEKLSKFSGGKLEDFVAYTLRKSLVVSKYDTQEFD |
| | SVAAMVVFLECIGKNNISDHEREIVCKLLELIRKDFSKLDPNVKGSQGANIVRSVRNQNMIVQPQGDRF |
| | LFPQVYAKENETVTNKNVEKEGLNEFLLNYANLDDEKRAESLRKLRRILDVYFSAPNHYEKDMDITLS |
| | DNIEKEKFNVWEKHECGKKETGLFVDIPDVLMEAEAENIKLDAVVEKRERKVLNDRVRKQNIICYRYT |
| | RAVVEKYNSNEPLFFENNAINQYWIHHIENAVERILKNCKAGKLFKLRKGYLAEKVWKDAINLISIKYI |
| | ALGKAVYNFALDDIWKDKKNKELGIVDERIRNGITSFDYEMIKAHENLQRELAVDIAFSVNNLARAVC |
| | DMSNLGNKESDFLLWKRNDIADKLKNKDDMASVSAVLQFFGGKSSWDINIFKEAYKGKKKYNYEVR |
| | FIDDLRKAIYCARNENFHFKTALVNDEKWNTELFGKIFERETEFCLNVEKDRFYSNNLYMFYQVSELR |
| | NMLDHLYSRSVSRAAQVPSYNSVIVRTAFPEYITNVLGYQKPGYDADTLGKWYSA |
| ULZH01.1 | MKLSKEKHTRSAVANEEQNIGGILYRFPGKSIDGVKDQMLRRDKEVKKLYNVFNQIQVGTKPKKWNN |
| SEQ ID NO: | DEKLSPEENERRAQQKNIKIENYKWREACSKYVKSSQKTINYVIFYSYGNAENKLRYMRKNEDILKKM |
| 4242 | QEEEKLPKFSGGKLEDFVAYTLRKSLWSKYDTQEFDSVAAMWFLECIGKNNISDHEREIVCKLLELI |
| | RKDFSKLDPNVKGSQGANIVRSVRNQNMIVQPQGDRFLFPQVYAKENETVTNKNVEKEGLNEFLLNY |
| | ANLDDEKRAESLRKLRRILDVYFSAPNHYEKDMDITLSDNIEKEKFNVLEKHECGKKETGLFVDIPDVL |
| | MEAEAENIKLDAVVEKRERKVLNDRVRKQNIICYRYTRAVVEKYNSNEPLFFENNAINQYWIHHIENA |
| | VERILKNCKAGKLFKLRKGYLAEKVWKDAINLISIKYIALGKAVYNFALDDIWKDKKNKELGIVDERIR |
| | NGITSFDYEMIKAHENLQRELAVDIAFSVNNLARAVCDMSNLGNKESDFLLWKRNDIADKLKNKDDM |
| | ASVSAVLQFFGGKSSWDINIFKEAYKGKKKYNYEVRFIDDLRKAIYCARNENFHFKTALVNDEKWNTE |
| | LFGKIFKRETEFCLNVEKDRFYSNNLYMFYQVSELRNMLDHLYSRSVSRAAQVPSYNSVIVRTAFPEYI |
| | TNVLGYQKPGYDADTLGKWYSACYYLLKEIYYNSFLQSDRALQLFEKSVKTLSWDDKKQQRAVDNF |
| | KDHFSDIKSACTSLAQVCQIYMTEYNQQNNQIKKVRSSNDSIFDQPIYQHYKVLLKKAIANAFADYLK |
| | NNKDLFGFIGKPFKANEIREIDKEQFLPDWTSRKYEALCIEVSGSQELQKWYIVGKFLNAMSLNLMVGS |
| | MRSYIQYVTDIKR |
| IMG_ | MKLSKEKYTRSAVANNGDIKSAEVNNGNTKSEEVNNEYIRSAVANEKQNIGGVLYHAHGTDTIDLQD |
| 3300014770 | QMLRRDKEVKKLYNVFNQIQVGTKPKKWNNDEKLSPEENERRAQQKNIKMKNYKWREACSKYVESS |
| SEQ ID NO: | QRIINDVIFYSYRKAENKLRYMRKNEDILKKMQEAEKLSKFSGGKLEDFVAYTLRKSLVVSKYDTQEF |
| 4243 | DSVAAMVVFLECIGKNNISDHEREIVCKLLELIRKDFSKLDPNVKGSQGANIVRSVRNQNMIVQPQGDR |
| | FLFPQVYAKENETVTNKNVEKEGLNEFLLNYANLDDEKRAESLRKLRRILDVYFSAPNHYEKDMDITL |
| | SDNIEKGKFNVWEKHECGKKVTDLFVDIPDVLMEAEAENIKLDAVVEKRERKVLTDRVRRQNIICYRY |
| | TRAVIEKYNSNEPLFFENNAINQYWIHHIENAVERILKNCKAGKLFKLRKGYLAEKVWKDAINLISIKYI |
| | ALGKAVYNFALDDIWKDKENKELGIVDERIRNGITSFDYEMIKAHENLQRELAVDIAFSVNNLARAVC |
| | DMSNLGNKESDFLLWKRNDIADKLKNKDDMASVSAVLQFFGGKSSWDINIFKEAYKGKKKYNYEVR |
| | FIDDLRKAIYCARNENFHFKTALVNDEKWNTELFGKIFERETEFCLNVEKDRFYSNNLYMFYQVSELR |
| | NMLDHLYSRSVSRAAQVPSYNSVIVRTAFPEYITNVLGYQKPGYDADTLGKWYSACYYLLKEIYYNSF |
| | LQSDRALQLFEKSVKTLSWDDKKQQRAVY |
| mgm4560421. | MKLSKEKYTRSAVANNGDIKSAEVNNGNTKSEEVNNEYIRSAVANEKQNIGGVLYHAHGTDTIDLQD |
| 3 | QMLRRDKEVKKLYNVFNQIQVGTKPKKWNNDEKLSPEENERRAQQKNIKMKNYKWREACSKYVESS |
| SEQ ID NO: | QRIINDVIFYSYRKAENKLRYMRKNEDILKKMQEAEKLSKFSGGKLEDFVAYTLRKSLVVSKYDTQEF |
| 4244 | DSVAAMVVFLECIGKNNISDHEREIVCKLLELIRKDFSKLDPNVKGSQGANIVRSVRNQNMIVQPQGDR |
| | FLFPQVYAKENETVTNKNVEKEGLNEFLLNYANLDDEKRAESLRKLRRILDVYFSAPNHYEKDMDITL |
| | SDNIEKGKFNVWEKHECGKKVTDLFVDIPDVLMEAEAENIKLDAVVEKRERKVLTDRVRRQNIICYRY |
| | TRAVIEKYNSNEPLFFENNAINQYWIHHIENAVERILKNCKAGKLFKLRKGYLAEKVWKDAINLISIKYI |
| | ALGKAVYNFALDDIWKDKENKELGIVDERIRNGITSFDYEMIKAHENLQRELAVDIAFSVNNLARAVC |
| | DMSNLGNKESDFLLWKRNDIADKLKNKDDMASVSAVLQFFGGKSSWDINIFKEAYKGKKKYNYEVR |
| | FIDDLRKAIYCARNENFHFKTALVNDEKWNTELFGKIFERETEFCLNVEKDRFYSNNLYMFYQVSELR |
| | NMLDHLYSRSVSRAAQVPSYNSVIVRTAFPEYITNVLGYQKPGYDADTLGKWYSACYYLLKEIYYNSF |
| | LQSDRALQLFEKSVKTLSWDDKKQQRAVYKIVDTVSDAKLY |
| OVTY01.1 | MKLSKEKHIRSAVANEEQNIGGVLYHVLGTDTIGLKDQMLIRDRDVKQLYNVFNQIQVGDKPKKWKN |
| SEQ ID NO: | DEKLSPEENERRAQQKNIKMKNYKWREACSKYVESSQRTINDVLFYSYMEADKKIRNMRKNEDILIK |
| 4245 | MQEVTKLPKFSGGKLEDFVAYTLRKSLVVSKNSTQEFDSVAAMVVFLECIGKSNISDHEKEIVCKLLEL |
| | IRKDFSKLDPNVEGSQGANIVRSVRNQNMIVQPQGDRFSFPQVSDKEKKTVTNKNVEKEGLNEFLLNY |
| | ANLDDEKRAEILRKLRRILDVYFSAPNHYEKDMDITLSDNIDKEKFNVWKKYECGKKVTDLFVDIPDV |
| | LMEAEAENIKLDAVVEKRERKVLADRVRRQNIICYRYTRAVVEKYNSNESLFFENDAINQYWIHHIEN |
| | AVERILKNCKAGKLFKLRMGYLAEKVWKDAINLISIKYIALGKAVYNFALDDIWKDKKDKELGIVDER |
| | IRNGITSFDYEMIKAYENLQRELSVDIAFSVNNLARAVCDMSNLKDRESDFLLWKKEDIADKLKNKDD |
| | MASVSAVLQFFGGKSSWDINIFKEAYKGKNKYNYEVRFIDDLRKAIYCARNENFHFKTALVNNEKWN |
| | TELFGKIFERETEFCLNVEKDRFYSNNLYMFYPVSELRNMLDQLYSRSVSRAAQVPSYNSVFIRKNFPE |
| | DITNVLRYQKPGYDADTLDKWYSACYYLL |
| OOCM01.1 | MKLSKEKHIRSAVANEEQNIGGVLYHVLGTDTIGLKDQMLIRDRDVKQLYNVFNQIQVGDKPKKWKN |
| SEQ ID NO: | DEKLSPEENERRAQQKNIKMKNYKWREACSKYVESSQRTINDVLFYSYMEADKKIRNMRKNEDILIK |
| 4246 | MQEVTKLPKFSGGKLEDFVAYTLRKSLVVSKNSTQEFDSVAAMVVFLECIGKSNISDHEKEIVCKLLEL |
| | IRKDFSKLDPNVEGSQGANIVRSVRNQNMIVQPQGDRFSFPQVSDKEKKTVTNKNVEKEGLNEFLLNY |
| | ANLDDEKRAEILRKLRRILDVYFSAPNHYEKDMDITLSDNIDKEKFNVWKKYECGKKVTDLFVDIPDV |
| | LMEAEAENIKLDAVVEKRERKVLADRVRRQNIICYRYTRAVVEKYNSNESLFFENDAINQYWIHHIEN |
| | AVERILKNCKAGKLFKLRMGYLAEKVWKDAINLISIKYIALGKAVYNFALDDIWKDKKDKELGIVDER |
| | IRNGITSFDYEMIKAYENLQRELSVDIAFSVNNLARAVCDMSNLKDRESDFLLWKKEDIADKLKNKDD |
| | MASVSAVLQFFGGKSSWDINIFKEAYKGKNKYNYEVRFIDDLRKAIYCARNENFHFKTALVNNEKWN |
| | TELFGKIFERETEFCLNVEKDRFYSNNLYMFYPVSELRNMLDQLYSRSVSRAAQVPSYNSVFIRKNFPE |
| | DITNVLRYQKPGYDADTLDKWYSACYYLL |
| OVGC01.1 | MKLSKEKHIRSAVANEEQNIGGVLYHVLGTDTIGLKDQMLIRDRDVKQLYNVFNQIQVGDKPKKWKN |
| SEQ ID NO: | DEKLSPEENERRAQQKNIKMKNYKWREACSKYVESSQRTINDVLFYSYMEADKKIRNMRKNEDILIK |
| 4247 | MQEVTKLPKFSGGKLEDFVAYTLRKSLVVSKNSTQEFDSVAAMVVFLECIGKSNISDHEKEIVCKLLEL |
| | IRKDFSKLDPNVEGSQGANIVRSVRNQNMIVQPQGDRFSFPQVSDKEKKTVTNKNVEKEGLNEFLLNY |
| | ANLDDEKRAEILRKLRRILDVYFSAPNHYEKDMDITLSDNIDKEKFNVWKKYECGKKVTDLFVDIPDV |
| | LMEAEAENIKLDAVVEKRERKVLADRVRRQNIICYRYTRAVVEKYNSNESLFFENDAINQYWIHHIEN |
| | AVERILKNCKAGKLFKLRMGYLAEKVWKDAINLISIKYIALGKAVYNFALDDIWKDKKDKELGIVDER |
| | IRNGITSFDYEMIKAYENLQRELSVDIAFSVNNLARAVCDMSNLKDRESDFLLWKKEDIADKLKNKDD |
| | MASVSAVLQFFGGKSSWDINIFKEAYKGKNKYNYEVRFIDDLRKAIYCARNENFHFKTALVNNEKWN |
| | TELFGKIFERETEFCLNVEKDRFYSNNLYMFYPVSELRNMLDQLYSRSVSRAAQVPSYNSVFIR |
| OOBZ01.1 | MKLSKEKHIRSAVANEEQNIGGVLYHVLGTDTIGLKDQMLIRDRDVKQLYNVFNQIQVGDKPKKWKN |
| SEQ ID NO: | DEKLSPEENERRAQQKNIKMKNYKWREACSKYVESSQRTINDVLFYSYMEADKKIRNMRKNEDILIK |
| 4248 | MQEVTKLPKFSGGKLEDFVAYTLRKSLVVSKNSTQEFDSVAAMVVFLECIGKSNISDHEKEIVCKLLEL |
| | IRKDFSKLDPNVEGSQGANIVRSVRNQNMIVQPQGDRFSFPQVSDKEKKTVTNKNVEKEGLNEFLLNY |
| | ANLDDEKRAEILRKLRRILDVYFSAPNHYEKDMDITLSDNIDKEKFNVWKKYECGKKVTDLFVDIPDV |
| | LMEAEAENIKLDAVVEKRERKVLADRVRRQNIICYRYTRAVVEKYNSNESLFFENDAINQYWIHHIEN |
| | AVERILKNCKAGKLFKLRMGYLAEKVWKDAINLISIKYIALGKAVYNFALDDIWKDKKDKELGIVDER |
| | IRNGITSFDYEMIKAYENLQRELSVDIAFSVNNLARAVCDMSNLKDRESDFLLWKKEDIADKLKNKDD |
| | MASVSAVLQFFGGKSSWDINIFKEAYKGKNKYNYEVRFIDDLRKAIYCARNENFHFKTALVNNEKWN |
| | TELFGKIFERETEFCLNVEKDRFYSNNLYMFYPVSELRNMLDQLYSRSVSRAAQVPSYNSVFIR |
| OKRX01.1 | MKLSKEKHIRSAVANEEQNIGGVLYHVPGTDTIDLKDQMLIRDRDVKQLYKVFNQIQVGNKPKKWKK |
| SEQ ID NO: | DEKLSPEENERRAQQKNIKMKNYKWREACSEYVESSQRTINDVLFYSYMEADKKIRNMRKNEDILKK |
| 4249 | MQEVTKLPKFSGGKLEDFVAYTLRKSLVVSKNSTQEFDSVAAMVVFLECIGKSNISDHEKEIVYKLLEL |
| | IRKDFSKLDPNVKDSQGANIVRSVRNQNMIVQPQGDRFLFPQVSDKEKKTVTNKNVEKEGLNEFLLNY |
| | ANLDDEKRAEILRKLRRILDVYFSAPNHYEKDMEITLSDNIDKEKFNVWKKYECGKKVTGLFVNIPDV |
| | LMEAEAENIKLDAVVEKRERKILADRVRRQNIICYRYTRAVVEKYNSNESLFFENDAINQYWIHHIENA |
| | VERILKNCKAGKLFKLRKGYLAEKVWKDAINLISIKYIALGKAVYNFALDDIWKDKKDKELGIVDERIR |
| | NGITSFDYEMIKAYENLQRELAVDIAFSVNNLARAVCDMSNLKDRESDFLLWKKEDIADKLKNKDDM |
| | ASVSAVLQFFGGKSSWDINIFKEAYKGKNKYNYEVRFIDDLRKAIYCARNENFHFKTALVNNEKWNTE |
| | LFGKIFERETEFCLNVEKDRFYSNNLYMFYPVSELRNMLDQLYSRSVSRAAQVPSYNSVFIRKNFPEDIT |
| | NVLRYQKPGYDADTLGKWYSACYYLLKEIYYNSFLQSDKALQLFEK |
| UERC01.1 | MKLSKEKHIRSAVANEEQNIGGVLYHVPGTDTIDLKDQMLIRDRDVKQLYKVFNQIQVGNKPKKWKK |
| SEQ ID NO: | DEKLSPEENERRAQQKNIKMKNYKWREACSEYVESSQRTINDVLFYSYMEADKKIRNMRKNEDILKK |
| 4250 | MQEVTKLPKFSGGKLEDFVAYTLRKSLVVSKNSTQEFDSVAAMVVFLECIGKSNISDHEKEIVYKLLEL |
| | IRKDFSKLDPNVKDSQGANIVRSVRNQNMIVQPQGDRFLFPQVSDKEKKTVTNKNVEKEGLNEFLLNY |
| | ANLDDEKRAEILRKLRRILDVYFSAPNHYEKDMEITLSDNIDKEKFNVWKKYECGKKVTGLFVNIPDV |
| | LMEAEAENIKLDAVVEKRERKILADRVRRQNIICYRYTRAVVEKYNSNESLFFENDAINQYWIHHIENA |
| | VERILKNCKAGKLFKLRKGYLAEKVWKDAINLISIKYIALGKAVYNFALDDIWKDKKDKELGIVDERIR |
| | NGITSFDYEMIKAYENLQRELAVDIAFSVNNLARAVCDMSNLKDRESDFLLWKKEDIADKLKNKDDM |
| | ASVSAVLQFFGGKSSWDINIFKEAYKGKNKYNYEVRFIDDLRKAIYCARNENFHFKTALVNNEKWNTE |
| | LFGKIFERETEFCLNVEKDRFYSNNLYMFYPVSELRNMLDQLYSRSVSRAAQVPSYNSVFIRKNFPEDIT |
| | NVLRYQKPGYDADTLGKWYSACYYLLKEIYYNSL |
| UESQ01.1 | MKLSKEKHIRSAVANEEQNIGGVLYHVPGTDTIDLKDQMLIRDRDVKQLYKVFNQIQVGNKPKKWKK |
| SEQ ID NO: | DEKLSPEENERRAQQKNIKMKNYKWREACSEYVESSQRTINDVLFYSYMEADKKIRNMRKNEDILKK |
| 4251 | MQEVTKLPKFSGGKLEDFVAYTLRKSLVVSKNSTQEFDSVAAMVVFLECIGKSNISDHEKEIVYKLLEL |
| | IRKDFSKLDPNVKDSQGANIVRSVRNQNMIVQPQGDRFLFPQVSDKEKKTVTNKNVEKEGLNEFLLNY |
| | ANLDDEKRAEILRKLRRILDVYFSAPNHYEKDMEITLSDNIDKEKFNVWKKYECGKKVTGLFVNIPDV |
| | LMEAEAENIKLDAVVEKRERKILADRVRRQNIICYRYTRAVVEKYNSNESLFFENDAINQYWIHHIENA |
| | VERILKNCKAGKLFKLRKGYLAEKVWKDAINLISIKYIALGKAVYNFALDDIWKDKKDKELGIVDERIR |
| | NGITSFDYEMIKAYENLQRELAVDIAFSVNNLARAVCDMSNLKDRESDFLLWKKEDIADKLKNKDDM |
| | VSVAASLPVE |
| ULSX01.1 | MKLSKEKQIRSAVANKEKNTEGVLYRFPGDDIGGVQAQMLVRDRDVKQLYNVFNQIQLGNKPKEWM |
| SEQ ID NO: | NDEKLSPEENERRAQQKNIKMKNYKWRKACSKYVESSQRAINDILFYSYKEADKKIRNMSKNEDILIK |
| 4252 | MQNAEKLSKFSSGKLEDFVAYTLRKSLVVSKYGNQEFDSIAAMVVFLECIGKSNISDHEKEIVYKLLDL |
| | IRKDFSKLDPSIQDSQGANIVRSIRNQNMIVQPQGDRFSFPQVSDEEKKTVTNKNVEKDGLNEFMLNYA |
| | NLDEEKRAEVLRKLRRILDVYFSAPSHYEKDMDITLSDNVNKGKFYVWKKHECGKKENGLFVDIPDV |
| | LVEAEAESIKLDAVVEKRERKVLADRVRRQNIICYRYTRAVVEKYNSNEPIFFENDTINQYWIHHIENA |
| | VERILKNCKTGTLFKLRKGYLAEKVWKDAINLISIKYIALGKAVYNFALDDIWKDKKDKKLGIVDERIR |
| | NGITSFDYEMIKAHENLQRELAVNIAFSVNNLARAVCDMSNLGDKESDFLLWKRNDIADKLKNKDDM |
| | ASVSAVLQFFGGKSSWDINIFKEAYKGKKKYNYEVQFIDDLRKAIYCARNENFHFKTALVNNEKWNT |
| | ELFGKIFERETEFCLNVEKDRFYSNNLYMFYPVSELRNMLDHLYSRSVSRAAQVPSYNSVLVRTVFPEY |
| | ITNVLRYQKPGYDADTLGKWYNACYYLLKEIYYNSFLQSDKALQLFEKSVRTLRWDDKKQQRAVDN |
| | FKNHFSDIKSACTSLAQVCQIYMTEYNQ |
| OLXW01.1 | LKWRKNMKLSKVTYRVKDKNAKYKKEYNVRAAVANNSENAGGVLYHVPGVDLIDLREQMLDRDRS |
| SEQ ID NO: | VRLLYNIFNHIQTGTKPKKWGNDETLSVDENERKAKEQNIKIMNYKWREACSEYIEKSQSTINSVLFYS |
| 4253 | YEESGYKTKRMNDNEAIIVKMQYENRLSHFTGGKLEDFVAYTLRNSLVVSRYDNQEFDSVNAMVVFI |
| | NNIGNGNISDKDKKTICKLADLIRNDFSKLNPNVQSSQGANMVRSVRNQNMVVQPQGDKVSFPLVSDE |
| | GKNTVTNKNVEKKGLNEFLLNYANLDDEERMEKLRKLRRIIDVYFSSPSHYQKDMDISLSDNIDKTKF |
| | DVWKKHETGKKNTELFVDIPDELLTAETEKIKLDAVLEKKARKRLTDSIRKQNMICYRYTRAVVEKYN |
| | STENLFFENDSINQYWIHHIENAVERILKSCKAETLFKLRRGYLTEKVWKDAINLISIKYIALGKVIYNFT |
| | VDDIWKDKKVKNLGSIDEKIKHGITSFDYEMIKAQEALQRELAVNVAFAANNLARAVCDMTNLKDKE |
| | SDFLIWNKKDIANKLKNKDDMASVSVVLQFFGGKSSWDIDAFREAYKGNKYNYEVCFIDDLRKAVYA |
| | ARNESFHFKTALVNNDIWNTEFFGKLFIKETEICLDIEKDRFYSNNLPVFYSD |
| OPHK01.12 | MKLSKVTYRVKDKNAKYKKEYNVRAAVANNSENAGGVLYHVPGVDLIDLREQMLDRDRSVRLLYNI |
| SEQ ID NO: | FNHIQTGTKPKKWGNDETLSVDENERKAKEQNIKIMNYKWREACSEYIEKSQSTINSVLFYSYEESGYK |
| 4254 | TKRMNDNEAIIVKMQYENRLSHFTGGKLEDFVAYTLRNSLVVSRYDNQEFDSVNAMVVFINNIGNGNI |
| | SDKDKKTICKLADLIRNDFSKLNPNVQSSQGANMVRSVRNQNMVVQPQGDKVSFPLVSDEGKNTVTN |
| | KNVEKKGLNEFLLNYANLDDEERMEKLRKLRRIIDVYFSSPSHYQKDMDISLSDNIDKTKFDVWKKHE |
| | TGKKNTELFVDIPDELLTAETEKIKLDAVLEKKARKRLTDSIRKQNMICYRYTRAVVEKYNSTENLFFE |
| | NDSINQYWIHHIENAVERILKSCKAETLFKLRRGYLTEKVWKDAINLISIKYIALGKVIYNFTVDDIWKD |
| | KKVKNLGSIDEKIKHGITSFDYEMIKAQEALQRELAVNVAFAANNLARAVCDMTNLKDKESDFLIWN |
| | KKDIANKLKNKDDMASVSVVLQFFGGKSSWDIDAFREAYKGNKYNYEVCFIDDLRKAVYAARNESF |
| | HFKTALVNNDIWNTEFFGKLFIKETEICLDIEKDRFYSN |
| OLNZ01.1 | LKWRKNMKLSKVTYRVKDKNAKYKKEYNVRAAVANNSENAGGVLYHVPGVDLIDLREQMLDRDRS |
| SEQ ID NO: | VRLLYNIFNHIQTGTKPKKWGNDETLSVDENERKAKEQNIKIMNYKWREACSEYIEKSQSTINSVLFYS |
| 4255 | YEESGYKTKRMNDNEAIIVKMQYENRLSHFTGGKLEDFVAYTLRNSLVVSRYDNQEFDSVNAMVVFI |
| | NNIGNGNISDKDKKTICKLADLIRNDFSKLNPNVQSSQGANMVRSVRNQNMVVQPQGDKVSFPLVSDE |
| | GKNTVTNKNVEKKGLNEFLLNYANLDDEERMEKLRKLRRIIDVYFSSPSHYQKDMDISLSDNIDKTKF |
| | DVWKKHETGKKNTELFVDIPDELLTAETEKIKLDAVLEKKARKRLTDSIRKQNMICYRYTRAVVEKYN |
| | STENLFFENDSINQYWIHHIENAVERILKSCKAETLFKLRRGYLTEKVWKDAINLISIKYIALGKVIYNFT |
| | VDDIWKDKKVKNLGSIDEKIKHGITSFDYEMIKAQEALQRELAVNVAFAANNLARAVCDMTNLKDKE |
| | SDFLIWNKKDIANKLKNKDDMASVSVVLQFFGGKSSWDIDAFREAYKGNKYNYEVCFIDDLRKAVYA |
| | ARNESFHFKTALVNNDIWNTEFFGKLFIKETEICLDIEKDRFYSNNLPVFYSDNDLKKMLDHLYNNKVS |
| | RAAQVPSYNSVMVRKYFPENITSTLKYQKPGYDEDTLEKWYSACYYLLKEIYYNSFLQSDEALALFEE |
| | SVNNLKGDNKDQEL |
| OYAA01.1 | LKWRKNMKLSKVTYRVKDKNAKYKKEYNVRAAVANNSENAGGVLYHVPGVDLIDLREQMLDRDRS |
| SEQ ID NO: | VRLLYNIFNHIQTGTKPKKWGNDETLSVDENERKAKEQNIKIMNYKWREACSEYIEKSQSTINSVLFYS |
| 4256 | YEESGYKTKRMNDNEAIIVKMQYENRLSHFTGGKLEDFVAYTLRNSLVVSRYDNQEFDSVNAMVVFI |
| | NNIGNGNISDKDKKTICKLADLIRNDFSKLNPNVQSSQGANMVRSVRNQNMVVQPQGDKVSFPLVSDE |
| | GKNTVTNKNVEKKGLNEFLLNYANLDDEERMEKLRKLRRIIDVYFSSPSHYQKDMDISLSDNIDKTKF |
| | DVWKKHETGKKNTELFVDIPDELLTAETEKIKLDAVLEKKARKRLTDSIRKQNMICYRYTRAVVEKYN |
| | STENLFFENDSINQYWIHHIENAVERILKSCKAETLFKLRRGYLTEKVWKDAINLISIKYIALGKVIYNFT |
| | VDDIWKDKKVKNLGSIDEKIKHGITSFDYEMIKAQEALQRELAVNVAFAANNLARAVCDMTNLKDKE |
| | SDFLIWNKKDIANKLKNKDDMASVSVVLQFFGGKSSWDIDAFREAYKGNKYNYEVCFIDDLRKAVYA |
| | ARNESFHFKTALVNNDIWNTEFFGKLFIKAVSYTHLRAHETSQ |
| OQHH01.1 | MDISLSDNIDKTKFDVWKKHETGKKNTGLFVDIPDELLTAETEKIKLDAVLEKQARKRLTDSIRKQNM |
| SEQ ID NO: | VCYRYTRAVVEKYNLTENLFFENDYINQYWIHHIENAVERILKSCKAETLFKLRMGYLTEKVWKDAIN |
| 4257 | LISIKYIALGKVIYNFAVDDIWKDKKVKNLGSIDEKIKHGITSFDYEMIKAQEALQRELAVNVAFAANN |
| | LARAVCDMTNLKDKESDFLIWNKKDIANKLKNKDDMASVSVVLQFFGGKSSWDIDAFREAYKGNKY |
| | NYEVCFIDDLRKAVYAARNESFHFKTALVNNDIWNTEFFGKLFIKETEICLDIEKDRFYSNNLPVFYSDN |
| | DLKKC |
| OGNS01.1 | MEADKKIRNMRKNEDILKKMQEVTKLPKFSGGKLEDFVAYTLRKSLVVSKNSTQEFDSVAAMVVFLE |
| SEQ ID NO: | CIGKSNISDHEKEIVYKLLELIRKDFSKLDPNVKDSQGANIVRSVRNQNMIVQPQGDRFLFPQVSDKEK |
| 4258 | KTVTNKNVEKEGLNEFLLNYANLDDEKRAEILRKLRRILDVYFSAPNHYEKDMEITLSDNIDKEKFNV |
| | WKKYECGKKVTGLFVNIPDVLMEAEAENIKLDAVVEKRERKILADRVRRQNIICYRYTRAVVEKYNS |
| | NESLFFENDAINQYWIHHIENAVERILKNCKAGKLFKLRKGYLAEKVWKDAINLISIKYIALGKAVYNF |
| | ALDDIWKDKKDKELGIVDERIRNGITSFDYEMIKAYENLQRELAVDIAFSVNNLARAVCDMSNLKDRE |
| | SDFLLWKKEDIADYAIMLEVYKKYGYCKFLAQKLGFYNYDIGKYTYRMINEEYGLENYLEKMVADE |
| | VVLLQQKDRSELISMINAKQDGKLLKKVATLNQVLEERELDYRIKEFETTRYIEDSDGNKKKKKYKNA |
| | WKIVRF |
| OQCX01.1 | MILLINIGYIILKMLLNVYLRVVKQKHCLKLRRGYLTEKVWKDAINLISIKYIALGKVIYNFTVDDIWKD |
| SEQ ID NO: | KKVKNLGSIDEKIKHGITSFDYEMIKAQEALQRELAVNVAFAANNLARAVCDMTNLKDKESDFLIWN |
| 4259 | KKDIANKLKNKDDMASVSVVLQFFGGKSSWDIDAFREAYKGNKYNYEVCFIDDLRKAVYAARNESF |
| | HFKTALVNNDIWNTEFFGKLFIKETEICLDIEKDRFYSNNLPVFYSDNDLKKMLDHLYNNKVSRAAQV |
| | PSYNSVMVRKYFPENITSTLKYQKPGYDEDTLEKWYSACYYLLKEIYYNSFLQSDEALALFEESVNNL |
| | KGDNKDQELAVKNFRNNYKNIKSSCTSFSQVCQMYMTEYNQQNNQFKKVRSSKDSIVDKPIYQHYKL |
| | LLKKVIANAFASYLQHNKELFGFIGKPLKVNCLKEIDKEQFLPEWTSKKYVSLCEEVRKSPELQKMVY |
| | CWKVFKFKVSKSYGRFYEILYTICK |
| IMG_ | LSKYLDYGTSDSGLSTWAELGRFCNDGEVNYGIYRDALNPIPNRNIVMSKLYGADTIIPKVINRVNEDII |
| 3300010998 | KEYYQMIKEIDQYRIKGKCDSEDEQKKLLHFQKIKNKIEFRDIVEYSELINDLLGQLINWSFLRERDLLY |
| SEQ ID NO: | FQLGFHYACLHNKSRKPEGYDIVKRNNGTTVKGTILRQIAGLYINGIGILDKTTSGDYKEAAQAGGSFG |
| 4260 | RFYSYSNKVMESTGFYAPDDEEGRKNSLYLAGLELFENLNEHESIVKKRNDIDHFKYYMGKAGSLLDL |
| | YSEVFDRFFTYDMKYQKNVINMLENILMRYFVIISPKVGSGTKLLDNNGKKERAQIEIISSGICSDEFSYE |
| | YSGGNVKTPARNTEFLNTVARILYYPEEIESYSLVKVQGEFSVTRTDGKNRYPEKKNGNNNNQKNRGN |
| | RQNYQRNKNHNNKKSSMSETVYTSSSPNESFGYNPFRDLPRDFKM |
| GCA_002349225.1_ | MVSDYFEDEDDYARYLAGFLDYESSLGDYSVSPSGMLKDFCRTAVDSSDDETINIYYDGENPILQRNIV |
| ASM234922v1_ | LAKLYGNGQIISDVLKANRVNVGDIQEYYRSKDKLTAYKTTGTFNSIDELKQIKKYQELKNHVEFIDIV |
| genomic | EYSEILNELQAQLVNYTFLRERDLLYFQLGFHFSCLKNDSYKPSDYVRIEAGDKVISNAILHQIASLYIN |
| SEQ ID NO: | GISLYIKDEADTYVKDKDKSAGGNIRVFFKYCKNTFTEYSDSQTVYSAGLELFENLDEHGQIIDLRNYID |
| 4261 | HFKYYISDKSVNSGRSMIDIYSEVFDRFFTYDLKYHKNIPNTLYNILMGHFIETNFDFSTGTKDXXXXXX |
| | XXXXXXXXXXXXXXXXXXXXXXXXXXXXXXXXX |
| IMG_3300024272 | MGDISKVSKGESVAFGTVNEGFETGISSFDYERMKAEDSLNRAMIKYISFAVNIFDASVRNPEQRTGGK |
| SEQ ID NO: | EDILLLKPENIVMYEDAVKRVLRYFGGISKFSESSLDVSDKNGFFTALKDELYAARNYAFHYVTGEAE |
| 4262 | KREKPVVTTLLDTEYMLVGSIFRKKYFSNNVPMFYRTADIDNLMSRLYKSNRVILAQMPSFNKVLSRN |
| | AVVDFANAYLAGDSKREMSQPEISEQFRSSFYFLLKEIYYYDFILKEDLLERFKNGVECAQASAIKKEN |
| | NSRKHVAMKNAYRDFMSRADKLTKTKGITFGQFCQEIMTEYNQQNSQKQKKPSAVEKTYVVKGQTR |
| | TSVREVEDKEQIYKHYRTLLYAGIREAFLIYLKEEAAFGFLRSPKDGREKFRDLKEEDFSQGWTTECYT |
| | KLKDAIIEDKELSSWYVTAHFMNQKHLNHLIGEIKNYVQFIDDIEKRAKVTGNRVCSTEEKMGKFTSLL |
| | EVLEFCKLFCGQVSNNLEDYFANNEEYAKYVAGFVDYGGTSAALLQAFCRENKELNYYDELNPIPNR |
| | NIILSLLYGNTAVLSSSMKKVTLQEVKGYQKNKESLSGVFKNGACKDENEQRKMSNYQKQKNRIEFV |
| | DVLTLTELLNDLYGQLISYSYLRERDLMFMQLGFYYTKLFHTSSVPAQDKLRVLSGDCDIKDGAVLYQ |
| | IAAMYSYDLPIYGISKQGVAVRKKSGVSTGAKLNQFSTEYCGGKWDIYTNGLYFFEDVDGRHKDYVE |
| | VRNYIEHFKYFADHKKSILDLYSDLYNGFFSYDTKLKKSMSFVLPNILLSHFVNAKLSYEKDVVQKNSE |
| | SYRRARIVIREKDIKSDFLTYKNKENSKAFYVPARNDVFLKEVLDMISFKR |
| IMG_ | MGDISKVSKGESVAFGTVNEGFETGISSFDYERMKAEDSLNRAMIKYISFAVNIFDASVRNPEQRTGGK |
| 3300018878 | EDILLLKPENIVMYEDAVKRVLRYFGGISKFSESSLDVSDKNGFFTALKDELYAARNYAFHYVTGEAE |
| SEQ ID NO: | KREKPVVTTLLDTEYMLVGSIFRKKYFSNNVPMFYRTADIDNLMSRLYKSNRVILAQMPSFNKVLSRN |
| 4263 | AVVDFANAYLAGDSKREMSQPEISEQFRSSFYFLLKEIYYYDFILKEDLLERFKNGVECAQASAIKKEN |
| | NSRKHVAMKNAYRDFMSRADKLTKTKGITFGQFCQEIMTEYNQQNSQKQKKPSAVEKTYVVKGQTR |
| | TSVREVEDKEQIYKHYRTLLYAGIREAFLIYLKEEAAFGFLRSPKDGREKFRDLKEEDFSQGWTTECYT |
| | KLKDAIIEDKELSSWYVTAHFMNQKHLNHLIGEIKNYVQFIDDIEKRAKVTGNRVCSTEEKMGKFTSLL |
| | EVLEFCKLFCGQVSNNLEDYFANNEEYAKYVAGFVDYGGTSAALLQAFCRENKELNYYDELNPIPNR |
| | NIILSLLYGNTAVLSSSMKKVTLQEVKGYQKNKESLSGVFKNGACKDENEQRKMSNYQKQKNRIEFV |
| | DVLTLTELLNDLYGQLISYSYLRERDLMFMQLGFYYTKLFHTSSVPAQDKLRVLSGDCDIKDGAVLYQ |
| | IAAMYSYDLPIYGISKQGVAVRKKSGVSTGAKLNQFSTEYCGGKWDIYTNGLYFFEDVDGRHKDYVE |
| | VRNYIEHFKYFADHKKSILDLYSDLYNGFFSYDTKLKKSMSFVLPNILLSHFVNAKLSYEKDVVQKNSE |
| | SYRRARIVIREKDIKSDFLTYKNKENSKAFYVPARNDVFLKEVLDMISFKR |
| mgm4547164.3_ | LXXXXXXXXXXXXXXXXXXXXXXXXXXXXXXXXPLKWYVLAHFLSPKHLNHLTGAFKSYGVFIND |
| 3 | IERRAGDTGNRTEKEIIRAESGRIKSIVDMLVFSSTFCGMTTNIIEDYFEDKEEYDKMLIRFVEQDKDNAS |
| SEQ ID NO: | EDVVVTKKSCGEKKHLIGIYYDAANPIINRNMIRALMYGDLRMLCQIWNTVTIREIKNYNKLKENLSG |
| 4264 | VFEKGTCTSKEEQKKLREFQSEKNRIELHDLLTFTEIISDLNGQLVNWSYFRERDLMYMQLGVQYTKLF |
| | FTNTIGPEDIRRKISGKGFSITDGAMLYQIVALYNFGLPLYGFDETKKGRIVSNAGASVGKTISKFITNYC |
| | DEDVYYEGLFFFENIGEHEAITETRNYIDHFKYYADHKRSLLDLYSEVYERFFNYSVNYRKSVSYILPNI |
| | LERYFIVLNTEMDKGERLGRNGKESRYHTVAGIRVKKVSSANFTYKLKVGNEEKKYQIPAHSGEFLTT |
| | VKKILEYKAEN |
| OPDA01.1 | MISFRNRKIRKRIYGNDFYGYWQEKESGQAKDGKEQKAWESFENRIDQIGRERSFGAICQGLMVEYML |
| SEQ ID NO: | QNRDISMVQTETGDGKTNKKQIYKHYRTLLYICIRSAFTEYLREKWEELRTPVLTVKEWSKEEFCQTD |
| 4265 | GLKHLSLFDHLKETFNDAESGSFWYMAAHFINQKYLNHLIGSIRNYLQFTEDIEDRALSLGDCVDNKRE |
| | EKNLRYRNTLEILEFVAQFCERTTNVMEDYFESNQEYAAYLSGFVDYNVSKKETDIEKALYGFCRQKF |
| | KVDGKEYMAGIYYDGENLIPNRNIIRANMYGNVSCLKPYMDRITLKEIRTMYADQNKLDIVLKEGVCR |
| | TEEEQKALKEFQNEKNRIELFDLCTYTQILNDMQAKLIGWSYMRERDLMYYQLGYYYTKLFWTDAIS |
| | EEDARRRLVGELVNVEDGVILYQILAFNSYNLPMIANKNNTVTFLKGEGSIGGKAITAFLKNYENAERI |
| | YEEALDLFENTDEHAAIINTRNYIEHFKYFIKSDRSMMDLYSEVYDRFFRHDHNRKKNVPDSLKNVLA |
| | DNFMIADISMELGSKKVGEKKKGFREHKSARIEFTDKGIRSTDMTYTVKPDPKDSKKDEKVLVPAHSE |
| | VFLKQFQKILEYRI |
| OYBV01.1 | LYSGEDLYKEIRKELYAIRNITFHYTTKADKDQTQKHDLAEYLFEEEFSDITELFREKYYANNVWKYY |
| SEQ ID NO: | DVEVINTIMENIYCGRKYRAAQVPAFKNIISRPELPQVMNGFVKGNSLRRLMNCPDRDVINKYWSALF |
| 4266 | FVLKELYYYDFLQEQKKPEDNVKERFFRAIEKLSGQENDDKKQKAWESFGNRIDQIGRDRSFGAICQG |
| | LMIEYMLQNSDISMVQTETDNGKANNKKQIYKHYRTLLYNCIREAFIEYLREKWEELRTPVLTVKEWS |
| | KEEFCRADGLKHLSLFDHLKKTFNDAESGSSWYMAAHFINQKYLNHLLGSIRNYLQFTEDIEDRAISLG |
| | DCVDNKREEKNLRYRNTLEILEFVAQFCERTTNVMEDYFESNQEYAEYLSGFVDYNTTKKETDIEKAL |
| | YSFCKQKFKVDGKEYMAGIYYDGENLIPNRNIIRANMYGNTSCLKPCMDRITLKEIRTMYADQNKLDL |
| | VLKEGVCHTEEEQKAYREYQNEKNRIELFDVCTYTQILNDMQARLIGWSYMRERDLMYYQLGYYYT |
| | KLFWTDSISEEDARRRLVGNLVNVEDGAILYQILAFNSYNLPIIANKNNTVTLLKDEGSIGGKAITAFFK |
| | NYENAEMIYEEALDLFENMDEHAAIINTRNYIEHFKYFIKSDRSMMDLYSEIYDRFFRHDHNRKKNVP |
| | DSLKNVLADNFMIVDIDMELGSKKVGEKKKGFREHKAARIEFTDSGIRSTDMTYTIKPDIKDNKKDKK |
| | VLVPARSEVFLKQFRKILEYRIQDKIQ |
| OQDP01.1 | LYSGEDLYKEIRKELYAIRNITFHYTTKAEKDQTQKHDLAEYLFEEEFSDITELFREKYYANNVWKYYD |
| SEQ ID NO: | AEVINTIMENIYCGRKYRAAQVPAFKNIISRPELPQVMNGFVKGNSLRRLMNCPDRDVINKYWSALFF |
| 4267 | VLKELYYYDFLQEQKRPEDNVKERFFRAIKKLSGQEKGDKEQKAWESFENRIDQIGRDRSFGAICQGL |
| | MIEYMLQNSDISMVQTETDNGKANNKKQIYKHYRTLLYNCISEAFIEYLREKWKELRTPVLTAKEWSK |
| | EEFCRVDGLKHLSLFDHLKETFNDAESGSSWYMAAHFINQKYLNHLLGSIRNYLQFTEDIEDRAISLGD |
| | CVDNKREEKNLRYRNTLEILEFVAQFCERTTNVMEDYFESNQEYAEYLSGFVDYNTTKKETDIEKALY |
| | SFCKQKFKVDGKEYMAGIYYDGENLIPNRNIIRANMYGNTSCLKPCMDRITLKEIRTMYADQNKLDM |
| | VLKEGVCHTEEEQKAYREYQNEKNRIELFDVCTYTQILNDMQARLIGWSYMRERDLMYYQLGYYYT |
| | KLFWTDSISEEDARRRLVGNLVNVEDGAILYQILAFNSYNLPIIANKNNTVTLLKDEGSIGGKAITAFFK |
| | NYENAEMIYEEALDLFENMDEHAAIINTRNYIEHFKYFIKSDRSMMDL |
| OQFB01.1 | MENIYCGRKYRAAQVPAFKNIISRPELPQVMNGFVKGNSLRRLMNCPDRDVINKYWSALFFVLKELYY |
| SEQ ID NO: | YDFLQEQKKPEDNVKERFFRAIEKLSGQENDDKKQKAWESFGNRIDQIGRDRSFGAICQGLMIEYMLQ |
| 4268 | NSDISMVQTETDNGKANNKKQIYKHYRTLLYNCIREAFIEYLREKWEELRTPVLTVKEWSKEEFCRAD |
| | GLKHLSLFDHLKKTFNDAESGSSWYMAAHFINQKYLNHLLGSIRNYLQFTEDIEDRAISLGDCVDNKR |
| | EEKNLRYRNTLEILEFVAQFCERTTNVMEDYFESNQEYAEYLSGFVDYNTTKKETDIEKALYSFCKQKF |
| | KVDGKEYMAGIYYDGENLIPNRNIIRANMYGNTSCLKPCMDRITLKEIRTMYADQNKLDLVLKEGVC |
| | HTEEEQKAYREYQNEKNRIELFDVCTYTQILNDMQARLIGWSYMRERDLMYYQLGYYYTKLFWTDSI |
| | SEEDARRRLVGNLVNVEDGAILYQILAFNSYNLPIIANKNNTVTLLKDEGSIGGKAITAFFKNYENAEMI |
| | YEEALDLFENMDEHAAIINTRNYIEHFKYFIKSDRSMMDLYSEIYDRFFRHDHNRKKNVPDSLKNVLA |
| | DNFMIVDIDMELGSKKVGEKKKGFREHKAARIEFTDSGIRSTDMTYTIKPDIKIIKMIKKFSYLHAQKYF |
| OGMW01.1 | MENILETISAKLIKGESIEELTQEALDKGISPKDILTKSLLEGMTRAGEMFKEKTLTMYDVLESAKNMEK |
| SEQ ID NO: | SVKILKPLLRDEDIVKKGKILTASVQGDFHDIGKNLCILMLESNGFQVIDMGVDVPQEKIEECIKKESPNI |
| 4269 | LMLSAMIAPTMEVMKMTIEYLREKWKELRTPVLTAKEWSKEEFCRVDGLKHLSLFDHLKETFNDAES |
| | GSSWYMAAHFINQKYLNHLLGSIRNYLQFTEDIEDRAISLGDCVDNKREEKNLRYRNTLEILEFVAQFC |
| | ERTTNVMEDYFESNQEYAEYLSGFVDYNTTKKETDIEKALYSFCKQKFKVDGKEYMAGIYYDGENLIP |
| | NRNIIRANMYGNTSCLKPCMDRITLKEIRTMYADQNKLDMVLKEGVCHTEEEQKAYREYQNEKNRIE |
| | LFDVCTYTQIQHDMQARLIGWSYMRERDLMYYQLGYYYTKLFWTDSISEEDARRRLVGNLVNVEDG |
| | AILYQILAFNSYNLPIIANKNNTVTLLKDVGSIGGKAITAFFKNYENAEMIYEEALDLFENMDEHAAIIN |
| | TRNYIEHFKYFIKSDRSMMDLYSEIYDRFFRHDHNRKKNVPDSLKNVLADNFMIVDIDMELGSKKVGE |
| | KKKGFREHKAARIEFTDSADMTYTIKPDIKDNKKVLVPARSEVFLKQFRKILEYRIQDKTQ |
| CEAE01.1 | MVAHFMTPKHLNHLRGEIKSYFAYIHGIEDRRYMAMGVRVPVNEVKRTQYRKILEILDLAAEYNGRIS |
| SEQ ID NO: | AKWEDYYTSEQEYAENIHQYLNFSNPHDRRDLKEQLRSFCNEKNNNSPSGYIGIFYNEKGPILNRNVAR |
| 4270 | ARMYGTEMILARALVNDKVQKEEILEYYRSLKMLKKNVFKKCKCENIGQEKKRRSYQQQKNRIELVD |
| | ILKYSEILNDLMSQLISWCYLRERDRMYFQIGFYYVALSAEASKIPEDSKLRILKGKGDSSGTEINITDNA |
| | VLYQMAAVYTYELPVYCLDEEGNAIVSRSAPRNTLTANGVRAFCQEYCREEWANKDTSIYENGLELF |
| | ESPQDERDIIELRNYIDHFKYYARRDRSILELYSKVFERFFKHDVKLKKSVTVILSNILARYFVIPKLSINY |
| | REEEEGEEKHKITEIDITELKTDVTIHKYEKKEADNQTKIYKKTLDYYNEKFLNRLKKVLTYNQG |
| mgm4547164. | LXXXXXXXXXXXXXXXXXXXXXXXXXXXXXXXXXXXXXXXXXXXXXXXXXXXXXXXXXXXXX |
| 3 | XXXXXXXXXXXXXXXXXXXXXXXDAESYFGEHQGISDEMKLASFCRQPIDELKADGTPQIIGLYHDG |
| SEQ ID NO: | TNEILNRNIVRASLYGTDKIIQGAADKVTETDIRDFYRMQTAVSQEELADRAKDQAEKAKRIKEVQNK |
| 4271 | KNRVELVNVKIYSDILNDLMTQLVSWAYFRERDLMYLALGAQYMRIFHGKKISEESVLRKLKWRDVV |
| | NIQEGAVLYQIVAMYTYHLPLYQVKYAADGRGIEEVKERIGMYGYKKDYFEKYCHREDILRPVLYFFE |
| | VEKDQEKIRSIRNYIDHFSYFVKADKSILDLYSDFYNMFFSYSENFRKSISFILPNILSKYFVLADIHLSKK |
| | TREAVTMNNVRVMRNCAGFDIDKELKSYQFTYNIKASVEDEDSTDGKEECTENTLNHIDEKTDLTTCK |
| | QSKILPVKIDARDAQFLKDIKQILRYSNV |
| OWDR01.1 | MAYQKLTKQRYYANNVGLFYRAEEIQELVQELYSQKNITEAQIPAFRTVLKRKDLPGYMEELGILFPD |
| SEQ ID NO: | NTQEKSKGDFEGTLYFLMKEIYYRDFIVKDKAAAYFFKAVDQNKEQSKKEDKHTERAAENFHRYVKS |
| 4272 | LEKKYNKKEISFGTVCQYIMMEYNQQNTTKQETEIYKHFKMLISLCIRKAFGNYIKETYRFLFHPIYSKQ |
| | QGEPEYLDTLELESGVKEKNYEWFTLAHFLHPVQLNHLVGDLKSYIQYREDILRRIVFAEQRVYADQQ |
| | KEVQQKVKTAKEILEVLEFVREVSGRVSNEYTDYYENEEEYAEFLYQYIDFRKREGKSAFESLKYFCQ |
| | NILDSGTVVDLYADTENPKVLRNIELTRMYAGSNVKIPEYEKITEDEIKMYYQEKNSVALILSRGLCRN |
| | EKEQKKVIEFNWKKKRLTLNEITDVFSLVNDLLGKMISFSYLRERSNVSSSWILLYGIMC |
| OHZY01.1 | VVDLYADTENPKVLRNIELTRMYAGSNVKIPEYEKITEDEIKMYYQEKNSVALILSRGLCRNEKEQKK |
| SEQ ID NO: | VIEFNWKKKRLTLNEIVNDLLGKMISFSYLRERDQMYLLLGFYYMALCAENKSENHLGWKGETLDKL |
| 4273 | ESSDSKFDIGGGLVLYQIVSAFNFGSKLLYISEDGRWKMAGGAFPGKYGRFENDYNHRTSLSKVIRLFE |
| | NESYEREIIYWRDYVDHMKYYVNQNQSIMEIYSAFYSKVLGYSAKLRKSVVFNLQAALEKHHINPECI |
| | WMTSDGKCADUCLMKNLESQKFTYKLAKREGEKTERKICMNALNENFLKTIRTSLEYKK |
| OLPG01.1 | MKISKVDHVKSGIDQKLSSQRGMLYKQPQKKYEGKQLEEHVRNLSRKAKALYQVFPVSGNSKMEKE |
| SEQ ID NO: | LQIINSFIKNILLRLDSGKTSEEIVGYINTYSVASQISGDHIQELVDQHLKESLRKYTCVGDKRIYVPDIIV |
| 4274 | ALLKSKFNSETLQYDNSELKILIDFIREDYLKEKQIKQIVHSIENNSTPLRIAEINGQKRLIPANVDNPKKS |
| | YIFEFLKEYAQSDPKGQESLLQHMRYLILLYLYGPDKITDDYCEEIEAWNFGSIVMDNEQLFSEEASMLI |
| | QDRIYVNQQIEEGRQSKDTAKVKKNKSKYRMLGDKIEHSINESVVKHYQEACKAVEEKDIPWIKYISD |
| | HVMSVYSSKNRVDLDKLSLPYLAKNTWNTWISFIAMKYVDMGKGVYHFAMSDVDKVGKQDNLIIGQ |
| | IDPKFSDGISSFDYERIKAEDDLHRSMSGYIAFAVNNFARAICSDEFRKKNRKEDVLTVGLDEIPLYDNV |
| | KRKLLQYFGGASNWDDSIIDIIDDKDLVACIKENLYVARNVNFHFAGSEKVQKKQDDILEEIVRKETRD |
| | IGKHYRKVFYSNNVAVFYCDEDIIKLMNHLYQREKPYQAQIPSYNKVISKTYLPDLIFMLLKGKNRTKI |
| | SDPSIMNMFRGTFYFLLKEIYYNDFLQASNLKEMFCEGLKNNVKNKTKKTKKI |
| mgm454716 | MIKNLIQDWIRALFXXXXXXXXXXXXXXXXXXXXXXXXXXXXXXXXXXXXXXXXXXXXXXXXXX |
| 4.3_2 | XXXXXXXXXXXXXXXXXXXXXXXXXXXXXXXXXXXXXXXXXXXKSADFIKKLRYIGEDINKEFGQ |
| SEQ ID NO: | LLKKGIEQYKNIEEPDHTIISKVYDYFGDSYIEAKALRKWDDLKDHEIEALIYVMVSYYLRKSLCGTIDL |
| 4275 | DEGKIRKVLGTDVSVHADTENAITCHTNVGDMVDKKSVSIRSTIQKLLISMLQQDPEQRRKMFGQIGK |
| | MDVYIFILVLHKDFSKIRQMQNLEKSIRNQNVPVQCRRIYSKKATAGRGGVSSEDKVVRLMPSSAVFD |
| | NQPINEISRRSYEFSFLQKYAAAEDKHDRDKILIEVNSLLVLFLYGEQAYGDRSEGKATDLEIVPCDQRE |
| | DWKHFSPDAYQKLNDYLSQDDKRASDSKVFWSSLKSELRKAMLIHYQESLRVLAGKYKAEGKKKEE |
| | WPAEMKEAMYWITWFEDCVERILRIQRANTRLSLYKLESGYLYKKSWREFLSFMGQKYIALGKAVYH |
| | IILPHNYMQGCKYDLGQVPAFYKERGITGFDYEYIKAVEALQRETSAYVASAAGNFIRSVSRQQDESKD |
| | LLLESQSAYFRNMTPENLSRAYIRVMRYFGGESAWQDWDALSKASDIEQFKRELLNIIRSCLYVLRNQS |
| | FHYAEGIVNELGGLSQDEAAVIESIIERRIDSISGVIREKYYSNNAWMFYADENIKGLLNVLYKKTGEIP |
| | AQVPSFHSACKKDQLLRLFMGETYKRAD |
| IMG_ | VFNIYRNFIDEIFEIIDKFDFKESNSISYLFPKFHKIYKRICKSENINDRQNGARKYLLQTIYYYYFQYEIEN |
| 3300008271 | NKNDIFKYLNLYKSKIKAKKISYSHIIDNKDVNNKNIEILYRNIQREGMLNNRLLQMNNEWIEFIMIQFIE |
| SEQ ID NO: | FISTKKLNWILDDLSEYRKKEKLDEKNKNKLLENLKQKMNKNITYSNDSYVFISLAQFLDLKEISNLIH |
| 4276 | DIKKFVQFREKNISKYKPEKNKVYIEELRKISYILKVMLEHKERVIQKHYLESYDEGISIMYGKDIEKKGI |
| | KDIKIKIKNENQEVINQPLYKSGNDENSSWIVLYGIEQAKRNGTFKFFKEFFNTNEDIKLCKEDIQKYEH |
| | LYNEKEENQRKVDEYISSEENNNIEEIKEINNNKKQLFYYYKNMINGDGLKKGYDFINDIYSHCLSWAY |
| | RIERDCSIYKVEAYNGKIKNFRNEIAHFNYFQKTDKSLLDLLNDFYNIFDYNLKYQRDVQKVINNIFEK |
| | YQVVREDGGPVIFYCKVDKKLKLSENLVPKKHSKYPEIELVHKKYVIFFKKLFEHKK |
| IMG_ | MKVVRPYGVSKTDHMRADVRVRRIHPNSFRNEAQDVANFAVSHSKLILAQWISLIDKVITKPSKGGAP |
| 3300005916 | SVDQFNLRNGIGESVWKLFLSKDLLNAPTTKRLKRLEREWWSKIHPYGSDIDPTGIMNFKGRWFKVFC |
| SEQ ID NO: | EDIEPAKVDFELIALALHDHLYSRERRLGESSTARARGLILARADSIGCNVLKERQQLLSFGTPWNNDEI |
| 4277 | KQYKTATNLVDELKSAAKKYEGQRAARTKRAIGQVFHNHYGKLFVDDDGKPINVAMAQRAFPGLFA |
| | LHEAAKSNIKRILKAPQDHWLKKFPDSPGSFMEGLSDDWRNKEINHLIRLGKVIHYEAAKLGGAYRPS |
| | QIFDNWSGNFSTSAFWSTDGQIAIKQSEAFVRNWRTIVSFASRSITNWADPNSAEEKDILGEREILRAVE |
| | SLSIAEFDRLASrYFGNSVERFATTSRAYRQDVMKLALLGLSRLRHSTFHFSGLSSFLDALHSLPEDCNE |
| | AVAEAVRGLYKDDINAHAQHLSEKLRSVDVERFLEQDQVDSLCHVLIDSEVYFNDLPNFQ SILHRGQD |
| | AYLFRKIDMRLPSVATRLSLNNQSTKCQYLILKLLYEGPFAKWLCDLPSDILHEFVKQHMDRATNEAR |
| | RIGENDKHIARAAGTIKIQENDTIFDLFSMLRRLSLSESRESQKQSVSKRNMGKYLRKLELDVIAQTFQY |
| | FVEANYFDWIFDLRVGPESYSF |
| IMG_ | MKVVRPYGVSKTDHMRADVRVRRIHPNSFRNEAQDVANFAVSHSKLILAQWISLIDKVITKPSKGGAP |
| 3300022856 | SVDQFNLRNGIGESVWKLFLSKDLLNAPTTKRLKRLEREWWSKIHPYGSDIDPTGIMNFKGRWFKVFC |
| SEQ ID NO: | EDIEPAKVDFELIALALHDHLYSRERRLGESSTARARGLILARADSIGCNVLKERQQLLSFGTPWNNDEI |
| 4278 | KQYKTATNLVDELKSAAKKYEGQRAARTKRAIGQVFHNHYGKLFVDDDGKPINVAMAQRAFPGLFA |
| | LHEAAKSNIKRILKAPQDHWLKKFPDSPGSFMEGLSDDWRNKEINHLIRLGKVIHYEAAKLGGAYRPS |
| | QIFDNWSGNFSTSAFWSTDGQIAIKQSEAFVRNWRTIVSFASRSITNWADPNSAEEKDILGEREILRAVE |
| | SLSIAEFDRLASrYFGNSVERFATTSRAYRQDVMKLALLGLSRLRHSTFHFSGLSSFLDALHSLPEDCNE |
| | AVAEAVRGLYKDDINAHAQHLSEKLRSVDVERFLEQDQVDSLCHVLIDSEVYFNDLPNFQSILHRGQD |
| | AYLFRKIDMRLPSVATRLSLNNQSTKCQYLILKLLYEGPFAKWLCDLPSDILHEFVKQHMDRATNEAR |
| | RIGENDKHIARAAGTIKIQENDTIFDLFSMLRRLSLSESRESQKQSVSKRNMGKYLRKLELDVIAQTFQY |
| | FVEANYFDWIFDLRVGPESYSF |
| IMG_ | MKVVRPYGVSKTDHMRADVRVRRIHPNSFRNEAQDVANFAVSHSKLILAQWISLIDKVITKPSKGGAP |
| 3300025642 | SVDQFNLRNGIGESVWKLFLSKDLLNAPTTKRLKRLEREWWSKIHPYGSDIDPTGIMNFKGRWFKVFC |
| SEQ ID NO: | EDIEPAKVDFELIALALHDHLYSRERRLGESSTARARGLILARADSIGCNVLKERQQLLSFGTPWNNDEI |
| 4279 | KQYKTATNLVDELKSAAKKYEGQRAARTKRAIGQVFHNHYGKLFVDDDGKPINVAMAQRAFPGLFA |
| | LHEAAKSNIKRILKAPQDHWLKKFPDSPGSFMEGLSDDWRNKEINHLIRLGKVIHYEAAKLGGAYRPS |
| | QIFDNWSGNFSTSAFWSTDGQIAIKQSEAFVRNWRTIVSFASRSITNWADPNSAEEKDILGEREILRAVE |
| | SLSIAEFDRLASrYFGNSVERFATTSRAYRQDVMKLALLGLSRLRHSTFHFSGLSSFLDALHSLPEDCNE |
| | AVAEAVRGLYKDDINAHAQHLSEKLRSVDVERFLEQDQVDSLCHVLIDSEVYFNDLPNFQSILHRGQD |
| | AYLFRKIDMRLPSVATRLSLNNQSTKCQYLILKLLYEGPFAKWLCDLPSDILHEFVKQHMDRATNEAR |
| | RIGENDKHIARAAGTIKIQENDTIFDLFSMLRRLSLSESRESQKQSVSKRNMGKYLRKLELDVIAQTFQY |
| | FVEANYFDWIFDLRVGPESYSF |
| IMG_ | MRIIRPYGRSAVIVQQVKDGTARERRIERNGAAQPEGKAAAGSPVQPVAALLAEGEPMLRQWLSIIDKI |
| 3300012994 | IAKPAADRAVKNIADARKRERLEKENREKQKIRQVREVLGQAVWEYLEAEQLLTEEEKQKLKEYWDS |
| SEQ ID NO: | KISAAAAKQGRRGDFPKGKLYRLFAGEAEWRDIDKNKAETIVNKIYRHLYGQACKIVPPHKAGADRA |
| 4280 | QYGHKQHSNQDKRAKSEGLIADRARAIAANVLQPRRVLANTDFSERDIERYRDKLRSHKDGDLAARIR |
| | QAVLAEQEKADKQPAYNIAVGELRAIWPVIFGEAKKYAEAQEADKALLAVHNALKEAYKQRLKGKP |
| | LFVDKQKAFLPQGAEAKNKLKELVVERLEKLLPADDEALFALLRNRRKNQDISHFIRLGKIIHYTAADK |
| | ARAAEADKAGADVTDFAGDETSFIARYWPKAEEVTDSRYWGSAGQTEIKQNEAFVRVWRRALAFAA |
| | RTVKDWVDPDNKIKGDILFSISEALSADRFNAAKAEHKYNLLFADDGDISDKKPSIAQQDWCFFALKA |
| | LYSLRNAAFHFKGMGEFVGALQKMGKLHEITGKETAEEKAKKEAENNIIKAVCPVLTKRYQQHRQKY |
| | QERLKDRLENIQLAYYLGGADIRFLWHKIATSNSSLLPLPRLRRVLERAEKAWKGGKQGEDSSGKLLP |
| | EIALPDYVPAQNREGEEGEAANCQWTVLKLLYDGAFKTWLDALPYGKVQGYIDEAICRATGAAQNLN |
| | KKNAKGEPKSAEELALTVSRNDGLYKLQPGDTIETFFYQLTAATATEMRVQNHYESNGAKAREQAAY |
| | IEDLKCDVVALALAAYLQVEDEGQRKNLAFVLDIPQRKKTENPSIDVHKQL |
| OQJI01.1 | MPSARGTDIYAREELRAEIASAFAAQAFGIDYTQNKYMENHEAYIQDYIKVLENEPNELFAAIKDAEKI |
| SEQ ID NO: | SDYLIEKGEFGLEKETEMSRDASFIKNMDTYVALHREYIEEVSQNKEPIVINGYGGPGAGKSTACMEIT |
| 4281 | AALKKEGYNAEYVQEYAKELVYEKDMEMLDGSPEHQYEILKEQTRRMDRLYDQVDFIVTDSPVMLN |
| | TIYNKQLTPEYESLVNELQGEYINYSFFMERDVSNFEEEGRIHNLTESIEKDNEIKDMLQKNEIKYKTYN |
| | HENVNEIVNDAIDFYEKINEGKSNEKEVVRDAENIQLTGAEAARFRMAMKGQERALDMFMNDESIPE |
| | HN |
| IMG_ | LAKAYRPAKQKKKHSYGAIRGAVQQIRNNVNHYKKDALNTIFNISEFENPSTTDNKNQTNYAETIYKN |
| 3300028769 | LFVTELKKIPEAFAQQLKTGGVLSYYTLDNLKTLLTTFQFSLCRSVIPFAPGFKKVFNGGINYQNATKDE |
| SEQ ID NO: | SFYELMLERYLPKENFAEEAYNARYFLLKLIYNNLFLPQFTTSKNAFADSVSFVQQQNKKQAEHSKRP |
| 4282 | KAFAFEAVRPMTNADSIAGYMAYVQSELMQEQNKKEEKATDETRINFEKFMLQVYIKGFDSFLKAQE |
| | FDFIQLPQPQLSATDNNQQKADKLNQLETAITGDCKLTPHYAKADVATHIAFYVFCKLLDAGHLSNLR |
| | NELIKFRESVDEFQFGHLLEIIEICLLSADIVPTDYRKLYSNEADCLARLTPFIEDGADITNWSDLFVQTD |
| | KHTPVIHANIELSVKYGTTKLLEQIVSKDPLFKTTEANFTAWNTAQKSIEQLIKQREDHHEKWVKAKN |
| | ADDKEKQEKRRDKSNWAQQYINEHGDDYLDICDYINTYNWLDNKMHFVHLNRLHSLTIELLGRMAG |
| | FVALFDRDFQFVDAQRSNDKFKIEEFVNLKRMDEKLNAVPRKKIKEIQDIRYKISQRNGNKIDESVRDIL |
| | IQSIHEKRNYYNSTFLLVNNDEKKENKVYDIRNHLAHFNYLTKNAADYSLLDLINELRELLNYDRKLK |
| | NAVSKAFIDLFDKHGMKLTLKLNAQHKLEVENLESKQLYHLGTSAKDKPEYRLTTNQVPTKYCAMCR |
| | SLLEMKN |
| IMG_ | LAKAYRPAKQKKKHSYGAIRGAVQQIRNNVNHYKKDALNTIFNISEFENPSTTDNKNQTNYAETIYKN |
| 3300028864 | LFVTELKKIPEAFAQQLKTGGVLSYYTLDNLKTLLTTFQFSLCRSVIPFAPGFKKVFNGGINYQNATKDE |
| SEQ ID NO: | SFYELMLERYLPKENFAEEAYNARYFLLKLIYNNLFLPQFTTSKNAFADSVSFVQQQNKKQAEHSKRP |
| 4283 | KAFAFEAVRPMTNADSIAGYMAYVQSELMQEQNKKEEKATDETRINFEKFMLQVYIKGFDSFLKAQE |
| | FDFIQLPQPQLSATDNNQQKADKLNQLETAITGDCKLTPHYAKADVATHIAFYVFCKLLDAGHLSNLR |
| | NELIKFRESVDEFQFGHLLEIIEICLLSADIVPTDYRKLYSNEADCLARLTPFIEDGADITNWSDLFVQTD |
| | KHTPVIHANIELSVKYGTTKLLEQIVSKDPLFKTTEANFTAWNTAQKSIEQLIKQREDHHEKWVKAKN |
| | ADDKEKQEKRRDKSNWAQQYINEHGDDYLDICDYINTYNWLDNKMHFVHLNRLHSLTIELLGRMAG |
| | FVALFDRDFQFVDAQRSNDKFKIEEFVNLKRMDEKLNAVPRKKIKEIQDIRYKISQRNGNKIDESVRDIL |
| | IQSIHEKRNYYNSTFLLVNNDEKKENKVYDIRNHLAHFNYLTKNAADYSLLDLINELRELLNYDRKLK |
| | NAVSKAFIDLFDKHGMKLTLKLNAQHKLEVENLESKQLYHLGTSAKDKPEYRLTTNQVPTKYCAMCR |
| | SLLEMKN |
| IMG_ | LAKAYRPAKQKKKHSYGAIRGAVQQIRNNVNHYKKDALNTIFNISEFENPSTTDNKNQTNYAETIYKN |
| 3300030002_2 | LFVTELKKIPEAFAQQLKTGGVLSYYTLDNLKTLLTTFQFSLCRSVIPFAPGFKKVFNGGINYQNATKDE |
| SEQ ID NO: | SFYELMLERYLPKENFAEEAYNARYFLLKLIYNNLFLPQFTTSKNAFADSVSFVQQQNKKQAEHSKRP |
| 4284 | KAFAFEAVRPMTNADSIAGYMAYVQSELMQEQNKKEEKATDETRINFEKFMLQVYIKGFDSFLKAQE |
| | FDFIQLPQPQLSATDNNQQKADKLNQLETAITGDCKLTPHYAKADVATHIAFYVFCKLLDAGHLSNLR |
| | NELIKFRESVDEFQFGHLLEIIEICLLSADIVPTDYRKLYSNEADCLARLTPFIEDGADITNWSDLFVQTD |
| | KHTPVIHANIELSVKYGTTKLLEQIVSKDPLFKTTEANFTAWNTAQKSIEQLIKQREDHHEKWVKAKN |
| | ADDKEKQEKRRDKSNWAQQYINEHGDDYLDICDYINTYNWLDNKMHFVHLNRLHSLTIELLGRMAG |
| | FVALFDRDFQFVDAQRSNDKFKIEEFVNLKRMDEKLNAVPRKKIKEIQDIRYKISQRNGNKIDESVRDIL |
| | IQSIHEKRNYYNSTFLLVNNDEKKENKVYDIRNHLAHFNYLTKNAADYSLLDLINELRELLNYDRKLK |
| | NAVSKAFIDLFDKHGMKLTLKLNAQHKLEVENLESKQLYHLGTSAKDKPEYRLTTNQVPTKYCAMCR |
| | SLLEMKN |
| IMG_ | LAKAYRPAKQKKKHSYGAIRGAVQQIRNNVNHYKKDALNTIFNISEFENPSTTDNKNQTNYAETIYKN |
| 3300031722 | LFVTELKKIPEAFAQQLKTGGVLSYYTLDNLKTLLTTFQFSLCRSVIPFAPGFKKVFNGGINYQNATKDE |
| SEQ ID NO: | SFYELMLERYLPKENFAEEAYNARYFLLKLIYNNLFLPQFTTSKNAFADSVSFVQQQNKKQAEHSKRP |
| 4285 | KAFAFEAVRPMTNADSIAGYMAYVQSELMQEQNKKEEKATDETRINFEKFMLQVYIKGFDSFLKAQE |
| | FDFIQLPQPQLSATDNNQQKADKLNQLETAITGDCKLTPHYAKADVATHIAFYVFCKLLDAGHLSNLR |
| | NELIKFRESVDEFQFGHLLEIIEICLLSADIVPTDYRKLYSNEADCLARLTPFIEDGADITNWSDLFVQTD |
| | KHTPVIHANIELSVKYGTTKLLEQIVSKDPLFKTTEANFTAWNTAQKSIEQLIKQREDHHEKWVKAKN |
| | ADDKEKQEKRRDKSNWAQQYINEHGDDYLDICDYINTYNWLDNKMHFVHLNRLHSLTIELLGRMAG |
| | FVALFDRDFQFVDAQRSNDKFKIEEFVNLKRMDEKLNAVPRKKIKEIQDIRYKISORNGNKIDESVRDIL |
| | IQSIHEKRNYYNSTFLLVNNDEKKENKVYDIRNHLAHFNYLTKNAADYSLLDLINELRELLNYDRKLK |
| | NAVSKAFIDLFDKHGMKLTLKLNAQHKLEVENLESKQLYHLGTSAKDKPEYRLTTNQVPTKYCAMCR |
| | SLLEMKN |
| GCA_900114365.1_ | VEFRDSIFKSLLQKEIEKAPLCFAEKLISGGVFSYYPSERLKEFVGNHPFSLFRKTMPFSPGFKRVMKSG |
| IMGtaxon_ | GNYQNANRDGRFYDLDIGVYLPKDGFGDEEWNARYFLMKLIYNQLFLPYFADAENHLFRECVDFVKR |
| 2651870357_ | VNRDYNCKNNNSEEQAFIDIRSMREDESIADYLAFIQSNIIIEENKKKETNKEGQINFNKFLLQVFVKGF |
| annotated_ | DSFLKDRTELNFLQLPELQGDGTRGDDLESLDKLGAVVAVDLKLDATGIDADLNENISFYTFCKLLDS |
| assembly_ | NHLSRLRNEIIKYQSANSDFSHNEDFDYDRIISIIELCMLSADHVSTNDNESIFPNNDKDFSGIRPYLSTDA |
| genomic_2 | KVETFEDLYVHSDAKTPITNATMVLNWKYGTDKLFERLMISDQDFLVTEKDYFVWKELKKDIEEKIKL |
| SEQ ID NO: | REELHSLWVNTPKGKKGAKKKNGRETTGEFSEENKKEYLEVCREIDRYVNLDNKLHFVHLKRMHSLL |
| 4286 | IELLGRFVGFTYLFERDYQYYHLEIRSRRNKDAGVVDKLEYNKIKDQNKYDKDDFFACTFLYEKANKV |
| | RNFIAHFNYLTMWNSPQEEEHNSNLSGAKNSSGRQNLKCSLTELINELREVMSYDRKLKNAVTKAVID |
| | LFDKHGMVIKFRIVNNNNNDNKNKHHLELDDIVPKKIMHLRGIKLKRQDGKPIPIQTDSVDPLYCRMW |
| | KKLLDLKPTPF |
| JMBV01.1 | VNADKLSHFFAEGVNVDDDENVIHASMETFRKYGTRDLFHKLMLQDDRFLVSSDDYREWEEMKEKIE |
| SEQ ID NO: | GGKVKQRELLHAEWCEAKEKDKKSRKVKSNSRTCFEKKFMGAKAEEYYSLCKVIDKYNWLDNKLHL |
| 4287 | VHLNKLHNLVIEILGRMVGFTALFERDFQYICKSDSEYEQLYNLDFNMGLPKFKNSIKGSGKAKNSTQ |
| | NIDHNATGIGNSSNLLKENSNGTHYCKNLSGDGVEDKLKRLFLYDDYRNVRNFVAHFNYLTRVEDDL |
| | GGNDAVKLSGTRYSLIELINELRNLLKYDRKLKNAVSKSFIDMFERHGMHVKMKLNHNHKLFVDSISP |
| | RKIKHLGGVVIRSGEG |
| GCA_000525995.1_ | MKLFGQLGVRFKKLEMKYTIVKSMLGKKILKIKGFEYRPNMKYADTEMKDLMDNDIAKIPVFIEEKLK |
| PRIP_MIRA_ | SSGVMRFYKQEDLQSIWERKQGFSLLTTNAPFVPSFKRVFAKGHDYQTSRNRKYDLALTIFDRLEYGE |
| assembly_ | EKFRARYFLTKLVYYQQFMPWFTTDSSAFREAANFVLHLNKNRQQDAKAFTNIREVEKNELPRDYMS |
| genomic_ | YVQGQIAIHEDATEDTPNHFEKFISQIFIKGFDKYMIASDLVFIQSPENQELEQSEIEEMRFDIQVTPSFLK |
| 2 | NKDDYISFWTFCKMLDAKHLSELRNEMIKYNGDLTEEQEIIGLALLGVDSRENDWTQFFSSEQEYEDV |
| SEQ ID NO: | MKGYVGDALYEREPYRQSDGKTPVLFRGVEQARKYGTETVIQRLFDANPEFKVSQSNIAEWERQKETI |
| 4288 | EGTIKRKKKFA |
| IMG_ | MAVLVSFAANSYYNLFGSASEDILGTEVVKNRRTNVIKVKSYIFKEKMLNYFFDSEDFDVNKTIEVLES |
| 3300008734 | ISYSIYNIRNGVGHFNKLVLGKYKKKDINTNKRVEEDLNNNEEIKGYFIKKRGEIEKKIKERFLSNNLQY |
| SEQ ID NO: | YYAKERIENYFEVYEFEILKEKIPFAPNFKRIIKKGENLLNNKKNKKYEYFKNFDKNSVEEKKEFLKTRN |
| 4289 | FLLKELYYNNFYKEFLSKKEEFKKIVIEVKEEKKNRGNNKKSGVSFQSIDDYDTKINISDYIASIHKKEM |
| | ERVEKYNEEKQKDTAKYIRDFVEEIFLTGFINYLEKDKRLHFLKEEFSILCNNNNVVDFNININEEKIKEF |
| | LKENDSKTLNLYLFFNMIDSKRISEFRNELIKYKQFTKKRLDEEKEFLGIKIELYETLIEFVILTREKLDTK |
| | KSEETDAWLVDKLYVKEKNECNEYEYKEYEEILKLFVDEKILSSKEAPYYATNNKTPILLSNFEKTRKY |
| | GTQSFLSEVQSNYKYSKVEKENIEDYNKKEEIEKKKKSNIEKLQDLKVELHKKWEQNKITEKEIKKYN |
| | DTIKEIREYNYLKNKEELQNVYLLHEILSDLLARNVAFFNKWERDFKFIVIAIKQFLRENDKEKVENFLN |
| | PPDNSKGKKVYFSVSKYKNTVENIDGIHKNFMNLIFLNNKFMNRKIDKMNCTIWVYFRNYIAHFLHLH |
| | TKNEKISLINQMNLLIKLFSYDKKVQNHILKSTKTLLEKYNIQINFEISNDKNEVFKYKIKNRLYSKKGK |
| | MLGKNNKFEILENEFLENVKAMLEYSE |
| IMG_ | MAVLVSFAANSYYNLFGSASEDILGTEVVKNRRTNVIKVKSYIFKEKMLNYFFDSEDFDVNKTIEVLES |
| 3300007648 | ISYSIYNIRNGVGHFNKLVLGKYKKKDINTNKRVEEDLNNNEEIKGYFIKKRGEIEKKIKERFLSNNLQY |
| SEQ ID NO: | YYAKERIENYFEVYEFEILKEKIPFAPNFKRIIKKGENLLNNKKNKKYEYFKNFDKNSVEEKKEFLKTRN |
| 4290 | FLLKELYYNNFYKEFLSKKEEFKKIVIEVKEEKKNRGNNKKSGVSFQSIDDYDTKINISDYIASIHKKEM |
| | ERVEKYNEEKQKDTAKYIRDFVEEIFLTGFINYLEKDKRLHFLKEEFSILCNNNNVVDFNININEEKIKEF |
| | LKENDSKTLNLYLFFNMIDSKRISEFRNELIKYKQFTKKRLDEEKEFLGIKIELYETLIEFVILTREKLDTK |
| | KSEETDAWLVDKLYVKEKNECNEYEYKEYEEILKLFVDEKILSSKEAPYYATNNKTPILLSNFEKTRKY |
| | GTQSFLSEVQSNYKYSKVEKENIEDYNKKEEIEKKKKSNIEKLQDLKVELHKKWEQNKITEKEIKKYN |
| | DTIKEIREYNYLKNKEELQNVYLLHEILSDLLARNVAFFNKWERDFKFIVIAIKQFLRENDKEKVENFLN |
| | PPDNSKGKKVYFSVSKYKNTVENIDGIHKNFMNLIFLNNKFMNRKIDKMNCTIWVYFRNYIAHFLHLH |
| | TKNEKISLINQMNLLIKLFSYDKKVQNHILKSTKTLLEKYNIQINFEISNDKNEVFKYKIKNRLYSKKGK |
| | MLGKNNKFEILENEFLENVKAMLEYSE |
| IMG_ | MLNYFFDSEDFDINKTIEVLESISYSIYNIRNGVGHFNKLVLGKYKKKDINTNKRVEEDLNNNEEIKGYF |
| 3300011981 | IQKRGEIEKKIKERFLSNNLQYYYAKERIENYFEVYEFEILKEKIPFAPNFKRIIKKGEDLFNNKKNKKYE |
| SEQ ID NO: | YFKNFDKNSAEEKKEFLKTRNFLLKELYYNNFYKEFLSKKEELKKIVIEVKEEKKNRGNNKKSGVSFQ |
| 4291 | NIDDYDTKINISDYIASIHKKEMERVEKYNEEKQKDTAKYIRDFVEEIFLTGFINYLEKDERLHFLKEEFS |
| | VLCNSNNNVIDFNVNINEEKIKEFLKENDSKTLNLYLFFNMIDSKRISEFRNELIKYKQFTKKRLDEEKE |
| | FLGIKIELYETLIEFVILTREKLDTKKSEETDAWLVDKLYVKEKNECNEYEYKEYEEILKLFVDEKILISK |
| | EAPYYATNNKTPIILSNFEKTRKYGTQNLLAKIQSSYKYNEIEKQKIENYNEKKESEKKKKSNIEKLQDL |
| | KVELHKKWEQNKITEKEIKKYNDTIKEIREYNYLKNKEELQNVYLLHEILSDLLARNVAFFNKWERDF |
| | KFIVIAIKQFLRENDKEKVENFLNPPDNSKGKKVYFSVSKYKNTVENIDGIHKNFMNLIFLNNKFMNRK |
| | IDKMNCTIWVYFRNYIAHFLHLHTKNEKISLINQMNLLIKLFSYDKKVQNHILKSTKTLLEKYNIQINFEI |
| | SNDKNEVFKYKIKNRLYSKKGKMLGKNNKFEILENEFLENVKAMLEYSE |
| UPKO01.1 | LKFLKKVLFIDDNNRISIEKLKSRIDDNFKNLLIQHVIEYGKIKYYVENDDYIRNIVKNGELKLETKDLEY |
| SEQ ID NO: | IKTKETLIRKMAVLVSFAANSYYNLFGSVSEDILGTEVVKNRRTNVIKVKSYIFKEKMLNYFFDSEDFDI |
| 4292 | NKTIEVLESISYSIYNVRNGVGHFNKLILGKYKKKDINTNKRVEEDLNNNEEIKGYFIQKRGEIEKKIKE |
| | RFLSNNLQYYYAKEKIENYFKVYEFEILKEKIPFAPNFKRIIKKGEDLFNDKNNKKYEYFKNFDKNNDD |
| | EKKEFLRTRNFLLKELYYNNFYKEFFSERKKYEFKKIITEVKEEKKNRGNNKKSGVSFQNIDDYDTKINI |
| | SDYIASIHKKEMERVEKYNEEKQKDTAKYIRDFVEEIFLTGFINYLEKDERLHFLKEEFSVLCNSNNNVI |
| | DFNVNINEEKIKEFLKENDSKTLNLYLFFNMIDSKRISEFRNELIKYKQFTKKRLDEEKEFLGIKIELYET |
| | LIEFVILTREKLDTKKSEETDVWLADKLYVKENNGYKEYEEILKLFVDEKILSSKEAPYYATDNKTPILL |
| | SNFEKIRKYGTQSFLSKIQSNYRYSEVEKQKIENNNEKKDSEKKKKSNIEKLQDLKVELHKKWEQNKIT |
| | EKEIEKYNDTIKEIREYNYLKNKEELQNVYLLHEILSDLLARNVAFFNKWERDFKFIVIAIKQFLRENDK |
| | EKVENFLNPPDNSKGKKVYFSVSKYKNTVENIDGIHKNFMNLIFLNNKFMNRKIDKMNCTIWVYFRNY |
| | IAHFLHLHTKNEKISLINQMNLLGRV |
| IMG_ | LKFLKKVLFIDDNNRISIEKLKSRIDDNFKNLLIQHVIEYGKIKYYVENDDYIKNIVKNGELKLETKDLEY |
| 3300006254 | IKTKETLIRKMALLVSFAVNSYYNLFGSVSEDILGTEVVKNRRTNVIKVKSYIFKEKMLNYFFDSEDFD |
| SEQ ID NO: | VNKTIEVLESISYSIYNIRNGVGHFNKLVLEKYKKKDIDTNKRVEEDLNNNKEIKGYFIKKRDEIEKKIK |
| 4293 | ERFLSNNLQYYYAKERIENYFEVYEFEILKEKIPFAPNFKRIIKKGEDLFNNKKNKKYEYFKNFDKNSAE |
| | EKKEFLKTRNFLLKELYYNNFYKEFLSKKEEFKKIVIEVKEEKKNRGNNKKSGVSFQSIDDYDTKINISD |
| | YIASIHKKEMERVEKYNEEKQKDTAKYIRDFVEEIFLTGFINYLEKDERLHFLKEEFSVLCNSNNNVIDF |
| | NVNINEEKIKEFLKENDSKTLNLYLFFNMIDSKRISEFRNELIKYKQFTKKRVDEEKEFLGIKIELYETLIE |
| | FVTLTREKLDTKKSEETDAWLADKLYVKENNGYKEYEEILKLFVDEKILSSKEAPYYATDNKTPILLSN |
| | FEKIRKYGTQSFLSKIQSNYRYSEVEKQKIENYNEKKESEKKKKSNIEKLQDLKVELHKKWEQNKITEK |
| | EUCKYNDTIKEIREYNYLKNKEELQNVYLLHEILSDLLARNVAFFNKWERDFKFIVIAIKQFLRENDKEK |
| | VNEFLNPPDDSKGKKVYFSVSKYKNTVENIDGIHKNFMNLIFLNNKFMNRKIDKMNCTIWVYFRNYIA |
| | HFLHLHTKNEKISLINQMNLLIKLFSYDKKVQNHILKSTKTLLEKYNIQINFEISNDKNEVFKYKIKNRL |
| | YSKKGKMLGKNNEFEILEKEFLKNVKAMLEYSE |
| UPKD01.1 | LYYNNFYKEFLSKKEEFKKIVIEVKEEKKNRGNNKKSGVSFQSIDDYDTKINISDYIASIHKKEMERVEK |
| SEQ ID NO: | YNEEKQKDTAKYIRDFVEETFLTGFINYLEKDKRLHFLKEEFSILCNNNNNVVDFNININEEKIKEFLKE |
| 4294 | NDSKTLNLYLFFNMIDSKRISEFRNELIKYKQFTKRRLDEEKEFLGIKIELYETLIEFVILTREKLDTKKSE |
| | EIDAWLVDKLYVKDNNEYKEYEEILKLFVDEKILSSKEAPYYATDNKTPILLSNFEKTRKYGTQSFLSEI |
| | QSNYKYSKVEKENIEDYNKKEEIEQKKKSNIEKLQDLKVELHKKWEQNKITEKEIKKYNDTIKEIREYN |
| | YLKNKEELQNVYLLHEMLSDLLARNVAFFNKWERDFKFIVIAIKQFLRENDKEKVNEFLNPHKSDGSR |
| | DNFSVTNYRSKMKSIINNIHENFMSLLFLNNNFTWGNLRNYIAHFEYLHKEKDTISFIGQANLLIKLFSY |
| | DKKVQNHIIKSMKTLLEKYNIEIRFEISNDSEEIFEYKIKYINSKKGKMLGKNNEFEILKNEFVRNVKALL |
| | EYSKL |
| UPCC01.1 | MGSGKLINKFSQHSIKSLISIFFSYLKCILLNPYYLFIPFNLSSKKTSATMEKQPRQILISATLNTGEKKESE |
| SEQ ID NO: | KKKKSNIEKLQDLKVELHKKWEQNKITEKEIEKYNDTIKEIREYNYLKNKEELQNVYLLHEILSDLLAR |
| 4295 | NVAFFNKWERDFKFIVIAIKQFLRENDKEKVNEFLNPHKSDGSKDNFSVTNYRSKMKSIINNIHENFMS |
| | LLFLNNNLATGGIQMGRNNNFTWGNLRNYIAHFEYLHKEKDTISFLNQANLLIKLFSYDKKVQNHIIKS |
| | MKTLLEKYNIEIGFEISNDSEEIFEYKIKYINSKKGKMLGKNNEFEILENEFVRNVKALLEYSKL |
| IMG_ | MNALFNKFYSSESEYDEKLKKFIEETILGDKKNTSFYSTDGKTPIVHSNLEKMRKYGTENFLSKVLKNS |
| 33000081612 | KYTLNNITAKEKFEAKVSDELKEYKILEKVNKFNKNEKRKKLIEYYNDCRCYLHKKWIENKKNKEEFE |
| SEQ ID NO: | YKEIYKKIIEEIRKYNYLENKEKLQNVYLLHEILSDLLARNVAFLNKWERDFKFIVIAIKQFLRENDKEK |
| 4296 | VDEFLNPHKSDGSRDNFSVTNYRSKIRLVINNIHENFMSLLFLNDNLATGGIQMGRNNNFTWGNLRNYI |
| | AHFEYLHKEKDTISFIGQANLLIKLFSYDKKVQNHIIKSMKTLLEKYNIEIRFEISNDSEEIFEYKIKYINSK |
| | KGKMLGKNNEFEILENEFVRNVKALLEYSE |
| IMG_ | MKGGSMKITKVDGLSHYKKQDKGILKKKWRDLDERKQREKIEERYNKQIESKIYKEFFRLKNKKRIEK |
| 3300008664 | EEDQNIKSLYFFIKEMYLNEENEEWELKNINLEILDDKERVIKGYKFKEDVYFFKEGDKKYYLRTLLNN |
| SEQ ID NO: | LIEKIQNENRDKVRKNKEFSDLKEIFKKYKDRKIKLLLESINNNKINLEYKKENVNEEIYGINPTNDREM |
| 4297 | TFHELLKEIIEKKDEQKSILEEKLDNFDITNFLENIEKIFNEETEINIIKGKVLNELREYIREKEENNSDYKL |
| | KQIYNLELKKYIENNFSYKKQKSKSKNGKNDYLYLNFLKKIMFIEEVDEKKGINKEKFKNKINSNFKNL |
| | FVQHILDYGKLLYYKENDEYIKNTGQLETKDLEYIKTKETLIRKMAVLVSFAANSYYNLFGRTENNILT |
| | QEISDDLLLGKIENEIYIKGEKNRRYVFKEKMLNYFFNPEIFGDNKIVEVLSAISSSIYNIRNGVNHFDKIN |
| | LGQYNNLDLSEIKKYFIEKRDKIKEKVKEKFSSNNLQYYYAKKEIENYFKAYEFEILKEKIPFAPNFKRII |
| | KKGEDLFNNKKNKKYEYFKNFDKNIAEEKKEFLKTRNFLLKELYYNNFYKEFLSKKEEFKKVVIEVKE |
| | EKKNRGNINNKKSGVSFQSIDDYDTKINISDYIASIHKKEMERVEKYNEEKQKDTAKYIRDFVEEIFLTG |
| | FINYLEKDKRLHFLKEEFSILCNNNNNVVDFNININEEKIKEFLKENDSKTLNLY |
| IMG_ | MKITKIDGVSHYKEKEKGVLKGKDILNGKIEKIVKKRYDATIESKIYKEFIKLRKNRIEQNNEKSILKLIK |
| 3300008408 | LNIDKNEKEIKTLLLNKFKIKEKNKKNDKYMLDENKLDNDIKIYESVESLYFLIKEIYLGQNNKKWNIS |
| SEQ ID NO: | KIDLEKIMEEDNNLIMLGYKLKKNITENDYPYLYSDKNGQESTSVYKLLKKLIEENKDRNQDIRKSQEY |
| 4298 | EKIRKNFEEYKNRKINLLVKSIKNNKINIQYINNEIKSHNNSREENIIKFFKKMIEEKNESILKDKLKLFKL |
| | EVFFDEEFLEEIKKLLDSDDFDKSYNKKISELRGKIFNRIREEIKNNKNRDELENIYFLELKKYIENNLSH |
| | KKEKNKNNNNTGEEKSKELYLKFKKKVLFIDDNNRINIEKLKSRIDDNFKNLLIQHVIEYGKIKYYVEN |
| | DDYIRNIVKNGELKLETKDLEYIKTKETLIRKMAVLVSFAVNSYYNLFGSVSEDILGTEVVKNRRTNVI |
| | KVKSYIFKEKMLNYFFDSEDFDVNKTIEVLESISYSIYNIRNGVGHFNKLVLEKYKKKDINTNKRVEED |
| | LNNNKEIKGYFIKKRDEIEKKIKERFLSNNLQYYYAKERIENYFEVYEFEILKEKIPFAPNFKRIIKKGEDL |
| | FNNKKNKKYEYFKNFDKNSAEEKKEFLKTRNFLLKELYYNNFYKEFLSKKEEFKKIVIEVKEEKKNRG |
| | NNKKSGVSFQSIDDYDTKINISDYIASIHKKEMERVEKYNEEKQKDTAKYIRDFVEEIFLTGFINYLEKD |
| | ERLHFLKEEFSVLCNSNNNVIDFNVNINEEKIKEFLKENDSKTLNLYLFFNMIDSKRISEFRNELI |

In some embodiments, the small Cas proteins are small Cas 13b. Examples of small Cas13b are shown in Table 2 below.

TABLE 2

| Accession | |
|---|---|
| No. | Sequences |
| GCA_ | MTEQNERPYNGTYYTLEDKHFWAAFLNLARHNAYITLTHIDRQLAYSKADITNDEDILFFKGQWKNLDND |
| 002206085.1_ | LERKARLRSLILKHFSFLEGAAYGKKLFENKSSGNKSSKNKELTKKEKEELQANALSLDNLKSILFDFLQKL |
| SJD4_genomic | KDFRNYYSHYRHPESSELPLFDGNMLQRLYNVFDVSVQRVKRDHEHNDKVDPHCHFNHLVRKGKKDRCG |
| SEQ ID NO: | NNDNPFFKHHFVDREGKVTEAGLLFFVSLFLEKRDAIWMQKKIRGFKGGTETYQQMTNEVFCRSRISLPKL |
| 4299 | KLESLRTDDWMLLDMLNELVRCPKSLYDRLREEDRARFRVPVDILSDEDDTDGTEEDPFKNTLVRHQDRF |
| | PYFALRYFDLKKVFTSLRFHIDLGTYHFAIYKKNIGEQPEDRHLTRNLYGFGRIQDFAEEHRPEEWKRLVRD |
| | LDYFETGDKPYITQTTPHYHIEKGKIGLRFVPEGQHLWPSPEVGATRTGRSKYAQDKRLTAEAFLSVHELM |
| | PMMFYYFLLREKYSEEASAERVQGRIKRVIEDVYAVYDAFARDEINTRDELDACLADKGIRRGHLPRQMIA |
| | ILSQEHKDMEEKVRKKLQEMIADTDHRLDMLDRQTDRKIRIGRKNAGLPKSGVIADWLX |
| GCA_ | MTEQNEKPYNGTYYTLEDKHFWAAFFNLARHNAYITLTHIDRQLAYSKADITNDEDILFFKGQWKNLDND |
| 002204455.1_ | LERKARLRSLILKHFSFLEGAAYGKKLFESQSSGNKSSKKKELTKKEKEELQANALSLDNLKSILFDFLQKL |
| ASM220445v1_ | KDFRNYYSHYRHPESSELPLFDGNMLQRLYNVFDVSVQRVKRDHEHNDKVDPHRHFNHLVRKGKKDRCG |
| genomic | NNDNPFFKHHFVDREEKVTEAGLLFFVSLFLEKRDAIWMQKKIRGFKGGTETYQQMTNEVFCRSRISLPKL |
| SEQ ID NO: | KLESLRTDDWMLLDMLNELVRCPKSLYDRLREEDRARFRVPVDILSDEDDTDGAEEDPFKNTLVRHQDRF |
| 4300 | PYFALRYFDLKKVFTSLRFHIDLGTYHFAIYKKNIGEQPEDRHLTRNLYGFGRIQDFAEEHRPEEWKRLVRD |
| | LDYFETGDKPYITQTTPHYHIEKGKIGLRFVPEGQHLWPSPEVGATRTGRSKYAQDKRLTAEAFLSVHELM |
| | PMMFYYFLLREKYSEEASAERVQGRIKRVIEDVYAVYDAFARGEIDTLDRLDACLADKGIRRGHLPRQMIA |
| | ILSQEHKDMEEKVRKKLQEMIADTDHRLDMLDRQTDRKIRIGRKNAGLPKSGVIADWLVRDMMRFQPVA |
| | KDTSGKPLNNSKANSTEYRMLQRALALFGGEKERLTPYFRQMNLTGGNNPHPFLHETRWESHTNILSFYRS |
| | YLKARKAFLQSIGRSDRVENHRFLLLKEPKTDRQTLVAGWKGEFHLPRGIFTEAVRDCLIEMGHDEVASY |
| UPHW01.1 | MTEQNEKPYNGTYYTLEDKHFWAAFLNLARHNAYITLTHIDRQLAYSKADITNDEDILFFKGQWKNLDND |
| SEQ ID NO: | LERKARLRSLILKHFSFLEGAAYGKKLFESQSSGNKSSKKKELTKKEKEELQANALSLDNLKSILFDFLQKL |
| 4301 | KDFRNYYSHYRHPESSELPLFDGNMLQRLYNVFDVSVQRVKRDHEHNHKVDPHRHFNHLVRKGKKDRY |
| | GNNDNPFFKHHFVDREEKVTEAGLLFFVSLFLEKRDAIWMQKKIRGFKGGTEAYQQMTNEVFCRSRISLPK |
| | LKLESLRTDDWMLLDMLNELVRCPKSLYDRLREEDRACFRVPVDILSVEDDTDGAEEDPFKNTLVRHQDR |
| | FPYFALRYFDLKKVFTSLRFHIDLGTYHFAIYKKNIGEQPEDRHLTRNLYGFGRIQDFAEEHRPEEWKRLVR |
| | DLDYFETGDKPYITQTTPHYHIEKGKIGLRFVPEGQHLWPSPEVGATRTGRSKYAQDKRLTAEAFLSVHEL |
| | MPMMFYYFLLRENYSDEASAERVQGRIKRVIEDVYAVYDAFARGEIDTLDRLDACLADKGIRRGHLPRQM |
| | IAILSQEHKDMEEKVRKKLQEMIADTDHRLDMLDRQTDRKIRIGRKNAGLPKSGVIADWLVRDMMRFQPV |
| | AKDTSGKPLNNSKANSTEYRMLQRALALFGGEKERLTPYFRQMNLTGGNNPHPFLHETRWESHTNILSFY |
| | RSYLEARKAFLQSIG |
| UPGW01.1 | MTEQNERPYNGTYYTLEDKHFWAAFLNLARHNAYITLAHIDRQLAYSKADITNDEDILFFKGQWKNLDND |
| SEQ ID NO: | LERKARLRSLILKHFSFLEGAAYGKKLFESQSSGNKSSKKKELTKKEKEVLQANALSLDNLKSILFDFLQKL |
| 4302 | KDFRNYYSHYRHPESSELPLFDGNMLQRLYNVFDVSVQRVKRDHEHNDKVDPHRHFNHLVRKGKKDRY |
| | GNNDNPFFKHHFVDREGKVTEAGLLFFVSLFLEKRDAIWMQKKIRGFKGGTETYQQMTNEVFCRSRISLPK |
| | LKLESLRTDDWMLLDMLNELVRCPKSLYDRLREEDRARFRVPVDILSDEDDTDGTEEDPFKNTLVRHQDR |
| | FPYFALRYFDLKKVFTS |
| UPIH01.1 | MTEQNERPYNGTYYTLEDKHFWAAFFNLARHNAYITLAHIDRQLAYSKADITNDEDILFFKGQWKNLDND |
| SEQ ID NO: | LERKARLRSLILKHFSFLEGAAYGKKLFESQSSGNKSSKKKELTKKEKEELQANALSLDNLKSILFDFLQKL |
| 4303 | KDFRNYYSHYRHPESSELPLFDGNMLQRLYNVFDVSVQRVKRDHEHNDKVDPHRHFNHLVRKGKKDKY |
| | GNNDNPFFKHHFVDREGTVTEAGLLFFVSLFLEKRDAIWMQKKIRGFKGGTETYQQMTNEVFCRSRISLPK |
| | LKLESLRTDDWMLLDMLNELVRCPKSLYDRLREEDRARFRVPVDILSDEDDTDGTEEDPFKNTLVRHQDR |
| | FPYFALRYFDLKKVFTSLRFHIDLGTYHFAIYKKNIGEQPEDRHLTRNLYGFGRIQDFAEEHRPEEWKRLVR |
| | DLDYFETGDKPYISQTTPHYHIEKGKIGLRFVPEGQHLWPSPEVGATRTGRSKYAQDKRLTAEAFLSVHEL |
| | MPMMFYYFLLREKYSDEASAEMVQGRIKRVIEDVYAVYDAFARDEINTRDELDACLADKGIRRGHLPRQ |
| | MIAILSQEHKDMEEKVRKKLQEMIADTDHRLDMLDRQTDRKIRIGRKNAGLPKSGVIADWLVRDMMRFQ |
| | PVAKDTSGKPLNNSKANSTEYRMLQRALALFGGEKERLTPYFRQMNLTGGNNPHPFLHETRWESHTNILSF |
| | YR |
| OWLX01.1 | MTEQNERPYNGTYYTLEDKHFWAAFLNLARHNAYITLAHIDRQLAYSKADITNDEDILFFKGQWKNLDND |
| SEQ ID NO: | LERKARLRSLILKHFSFLEGAAYGKKLFESQSSGNKSSKKKELTKKEKEELQANALSLDNLKSILFDFLQKL |
| 4304 | KDFRNYYSHYRHPESSELPMFDGNMLQRLYNVFDVSVQRVKRDHEHNDKVDPHRHFNHLVRKGKKDRC |
| | GNNDNPFFKHHFVDREGTVTEAGLLFFVSLFLEKRDAIWMQKKIRGFKGGTEAYQQMTNEVFCRSRISLPK |
| | LKLESLRTNDWMLLDMLNELVRCPKSLYDRLREEDRARFRVPVDILSDEDDTDGTEEDPFKNTLVRHQDR |
| | FPYFALRYFDLKKVFTSLRFHIDLGTYHFAIYKKNIGEQPEDRHLTRNLYGFGRIQDFAEEHRPEEWKRLVR |
| | DLDYFETGDKPYITQTTPHYHIEKGKIGLRFVPEGQLLWPSPEVGATRTGRSKYAQDKRFTAEAFLSVHEL |
| | MPMMFYYFLLREKYSEEVSAEKVQGRIKRVIEDVYAVYDAFARDEINTRDELDACLADKGIRRGHLPRQM |
| | IAILSQEHKDMEEKVRKKLQEMMADTDHRLDMLDRQTDRKIRIGRKNAGLPKSGVIADWLVRDMMRFQP |
| | VAKDTSGKPLNNSKANSTEYRMLQRALALFGGEKERLTPYFRQMNLTGGNNPHPFLHETRWESHTNILSF |
| | YRSYLKARKAFLQSIGRSDRVENHRFLLLKEPKTDRQTLVAGWKGEFHLPRGIFTEAVRDCLIEMGYDEVG |
| | SYKEVGFMAKAVPLYFERASKDRVLNLNQLPLIFFQSD |
| OWLO01.1_2 | MTEQNERPYNGTYYTLEDKHFWAAFFNLARHNAYITLAHIDRQLAYSKADITNDEDILFFKGQWKNLDND |
| SEQ ID NO: | LERKARLRSLILKHFSFLEGAAYGKKLFENKSSGNKSSKKKELTKKEKEELQANALSLDNLKSILFDFLQKL |
| 4305 | KDFRNYYSHYRHPESSELPLFDGNMLQRLYNVFDVSVQRVKRDHEHNDKVDPHRHFNHLVRKGKKDRY |
| | GNNDNPFFKHHFVDREEKVTEAGLLFFVSLFLEKRDAIWMQKKIRGFKGGTETYQQMTNEVFCRSRISLPK |
| | LKLESLRTDDWMLLDMLNELVRCPKSLYDRLREEDRACFRVPVDILSDEDDTDGAEEDPFKNTLVRHQDR |
| | FPYFALRYFDLKKVFTSLRFHIDLGTYHFAIYKKNIGEQPEDRHLTRNLYGFGRIQDFAEEHRPEEWKRLVR |
| | DLDYFETGDKPYLRLRLIQSLGKRTHLHLLPPS |
| GCA_ | MTEQNEKPYNGTYYTLEDKHFWAAFLNLARHNAYITLTHIDRQLAYSKADITNDEDILFFKGQWKNLDND |
| 000503975.1_ | LERKARLRSLILKHFSFLEGAAYGKKLFESQSSGNKSSKKKELTKKEKEELQANALSLDNLKSILFDFLQKL |
| SJD2_ | KDFRNYYSHYRHPESSELPLFDGNMLQRLYNVFDVSVQRVKRDHEHNHKVDPHRHFNHLVRKGKKDKY |
| genomic_2 | GNNDNPFFKHHFVDREGKVTEAGLLFFVSLFLEKRDAIWMQKKIRGFKGGTETYQQMTNEVFCRSRISLPK |
| SEQ ID NO: | LKLESLRTDDWMLLDMLNELVRCPKSLYDRLREEDRARFRVPVDILSDEDDTDGTEEDPFKNTLVRHQDR |
| 4306 | FPYFAILRSEESLHFPPLPYRFGHLPLRHIQEEHRRAAGRPPSDAQPVRLRPNTGFRRGA |
| GCA_ | MTEQNEKPYNGTYYTLEDKHFWAAFLNLARHNAYITLTHIDRQLAYSKADITNDEDILFFKGQWKNLDND |
| 002206065.1_ | LERKARLRSLILKHFSFLEGAAYGKKLFESQSSGNKSSKKKELTKKEKEELQANALSLDNLKSILFDFLQKL |
| SJD5_ | KDFRNYYSHYRHPESSELPLFDGNMLQRLYNVFDVSVQRVKRDHEHNHKVDPHRHFNHLVRKGKKDKY |
| genomic_2 | GNNDNPFFKHHFVDREGKVTEAGLLFFVSLFLEKRDAIWMQKKIRGFKGGTETYQQMTNEVFCRSRISLPK |
| SEQ ID NO: | LKLESLRTDDWMLLDMLNELVRCPKSLYDRLREEDRARFRVPVDILSDEDDTDGTEEDPFKNTLVRHQDR |
| 4307 | FPYFAILRSEESLHFPPLPYRFGHLPLRHIQEEHRRAAGRPPSDAQPVRLRPNTGFRRGA |
| IMG_ | MENDKKLEESACYTLNDKHFWAAFLNLARHNVYITVNHINKTLGEGEINRDGYETTLENTWNEIKDINKK |
| 3300011985 | ARLRELIIKHFPFLEAATYQQRSTDSTKQKEEKQAEAQSLESLKHCLFPFLKKLQKSRDHYSHYKHSKSLER |
| SEQ ID NO: | PKFEEDLQKKMYNIFDVSIRLVKEDYKHNTDINLKEDFKHLNRTGKFKYSFADNKGNITESGLLFFISLFLEK |
| 4308 | KDAIWMQKKLKGFKDSREKYQKMTNEVFCRSRILLPKLRLESTQTQDWILLDMLNELIRCPKSLYERLREK |
| | ERKEFKVPIEIADEDYDAEQEPFRNTLVRHQDRFPYFALRYFDYNEIFTNLRFQIDLGTYHFSIYKKLIGGNK |
| | EDRHLTHKLYGFERIQEFAKQNRPDEWKALVKDLDTFNKEEEKLYISETTPHYHLENEKIGIVFKNHNIWPS |
| | TQTELTNNNRKKYNLGVSIKAEAFLSVHELLPMMFYYLLLKTKNTHNGNEVEAKKKGTKNKKQEKHKIE |
| | AIIESKIKDIYNLYDAFANGEINSIEELEEHCKGKDIEIGHLPKQMIAILKDEHKDMAKKAETKQEKMILATE |
| | NRLKTLDKKLKGKIRNGKRCNSALKSGEIASWLVNDMMRFQPV |
| GCA_ | MEDDKKTTDSISYELKDKHFWAAFLNLARHNVYITVNHINKILEEDEINRDGYENTLENSWNEIKDINKKD |
| 002204405.1_ | RLSKLIIKHFPFLEAATYRQNPTDTTKQKEEKQAEAQSLESLKKSFFVFIYKLRDLRNHYSHYKHSKSLERPK |
| ASM220440v1_ | FEEGLLEKMYNIFNASIRLVKEDYQYNKDINPDEDFKHLDRTEEEFNYYFTKDNEGNITESGLLFFVSLFLEK |
| genomic | KDAIWLQQKLRGFKDNRESKKKMTNEVFCRSRMLLPKLRLESTQTQDWILLDMLNELIRCPKSLYERLQG |
| SEQ ID NO: | EDREKFRVPIEIADEDYDAEQEPFKNTLVRHQDRFPYFALRYFDYNEIFTNLRFQIDLGTYHFSIYKKLIGGQ |
| 4309 | KEDRHLTHKLYGFERIQEFDKQNRPDEWKAIVKDSDTFKKKEEKEEEKPYISETTPHYHLENKKIGIAFKNH |
| | NIWPSTQTELTNNKRKKYNLGTSIKAEAFLSVHELLPMMFYYPVVKDGKY |
| QWCT01.1 | VKMEDDKKTTDSISYALKDKHFWAAFLNLARHNVYITVNHINKILEEDEINRDGYENTLENSWNEIKDINK |
| SEQ ID NO: | KDRLSKLIIKHFPFLEAATYRQNPTDTTKQKEEKQAEAQSLESLKKSFFVFIYKLRDLRNHYSHYKHSKSLE |
| 4310 | RPKFEEDLQNKMYNIFDVSIQFVKEDYKHNTDINPKKDFKHLDRKRKGKFHYSFADNEGNITESGLLFFVSL |
| | FLEKKDAIWVQKKLEGFKCSNESYQKMTNEVFCRSRMLLPKLRLESTQTQDWILLDMLNELIRCPKSLYER |
| | LQGVNRKKFYVSFDPADEDYDAEQEPFKNTLVRHQDRFPYFALRYFDYNEIFTNLRFQIDLGTYHFSIYKKL |
| | IGGQKEDRHLTHKLYGFERIQEFDKQNRPDEWKAIVKDSDTFKKKEEKEEEKPYISETTPHYHLEN |
| IMG_ | MENDKRLEESACYTLNDKHFWAAFLNLARHNVYITINHINKLLEIRQIDNDEKVLDIKALWQKVDKDINQK |
| 3300008152 | ARLRELMIKHFPFLEFAIYNNNKDGKQEEKQAKAQSFESLKDCLFLFLKKLQESRNYYSHYKYSESSQEPKL |
| SEQ ID NO: | EKELRKKMYNIFDASIRLVKEDYQYNKDIDPEKDFKHLERKEDFNYLFTDKDNKGKITKNGLLFFVSLFLE |
| 4311 | KKDAIWMQQKLRGFKDNRGNKEKMTHEVFCRSRMLLPKIRLESTQTQDWILLDMLNELIRCPKSLYERLQ |
| | GAYREKFKVPFDSIDEDYDAEQEPFRNTLVRHQDRFPYFALRYFDYNEIFKNLRFQIDLGTYHFSIYKKLIGG |
| | QKEDRHLTHKLYGFERIQEFAKQNRPDEWKAIVKDLDTYETSNERYISETTPHYHLENQKIGIRFRNGNKEI |
| | WPSLKTNGENNEKSKYKLDKQYQAEAFLSVHELLPMMFYYLLLKKEEPNNDKKNASIVEGFIKREIR |
| UPIY01.1 | VRMENDKRLEESTCYTLNDKHFWAAFLNLARHNVYITINHINKLLEIRQIDNDEKVLDIKALWQKVDKDIN |
| SEQ ID NO: | QKARLRELMIKHFPFLEFAIYNNNKDGKQEEKQAKAQSFESLKRCLFLFLEKLQEARNYYSHYKYSESSKE |
| 4312 | PEFEEGLLEKMYNIFDENIQLVINDYQHNKDINPEKDFKHLDRTEEEFNYYFTKDKKGNITESGLLFFVSLFL |
| | EKKDAIWMQQKFRGFKDNRGNKEKMTHEVFCRSRMLLPKIRLESTQTQDWILLDMLNELIRCPKSLYERL |
| | QGAYREKFKVPFDSIDEDYDAEQEPFRNTLVRHQDRFPYFALRYFDYNEIFKNLRFQIDLGTYHFSIYKKLIG |
| | GQKEDRHLTHKLYGFERIQEFAKQNRPDEWKAIVKDLDTYETSNERYISETTPHYHLENQKIGIRFRNGNKE |
| | IWPSLKTNGENNEKSKYKLDKQYQAEAFLSVPELLPMMFYYLLLKKEEPNNDKKNASIVEGFIKREIRYIYK |
| | LYDAFANGEINNIDDLEKYCEDKGIPKRHLPKQMVAILYDEHKDMVEEAKRKQKEMVKDTKKLLATLEK |
| | QTQGEIEDGGRNIRLLKSGEIARWLVNDMMRFQPVQKDNEGNPLNNSKANSTEYQMLQRSLALYNKEEKP |
| | TRYFRQVNLINSSNPHPFLKWTKWEECNNILSFYRSYLTKKIEFLNKLKPEDWEKNQYFLKLKEPKTNRETL |
| | VQGWKNGFNLPRGIFTEPIREWFKRHQNDSKEYKNVEALDRVGLVTKVIPLFFKEEYFKEDAQKEINNCVQ |
| | PFYSFPYNVGNIHKPDEKDFLPSEERKKLWGDKKYKFKGYKAKVKSKKLTDKEKEEY |
| OWLO01.1 | VKMEDDKKTTESTNMLDNKHFWAAFLNLARHNVYITVNHINKVLELKNKKDQDIIIDNDQDILAIKTHWE |
| SEQ ID NO: | KVNGDLNKTERLRELMTKHFPFLETAIYTKNKEDKEEVKQEKQAKAQSFDSLKHCLFLFLEKLQEARNYY |
| 4313 | SHYKYSESTKEPMLEKELLKKMYNIFDDNIQLVIKDYQHNKDINPDEDFKHLDRTEEEFNYYFTRNKKGNI |
| | TASGLLFFVSLFLEKKDAIWMQQKLRGFKDNRESKKKMTHEVFCRRRMLLPKLRLESTQTQDWILLDMLN |
| | ELIRCPKSLYERLQGEDREKFKVPFDPADEDYDAEQEPFKNTLVRHQDRFPYFALRYFDYNEIFTNLRFQID |
| | LGTYHFSIYKKLIGGQKEDRHLTHKLYGFERIQEFNKQNRPDE |
| OWLL01.1 | MEDDKKTKESTNMLDNKHFWAAFLNLARHNVYITVNHINKVLELKNKKDQDIIIDNDQDILAIKTHWEKV |
| SEQ ID NO: | DGDLNKTERLRELMTKHFPFLETAIYTKNKEDKEEVKQEKQAEAQSLESLKDCLFLFLEKLQEARNYYSHY |
| 4314 | KYSEFSKEPEFKEELLEKMYNIFDANIQLVINDYQHNKDIDPEEDFKHLDTIEDSSYSFTVKDNKEKITASGL |
| | LFFVSLFLEKKDAIWMQQKLRGFKDNRESKKKMTHEVFCRSRILLPKLRLESTQTQDWILLDMLNELIRCP |
| | KSLYERLQGEDREKFKVPFDPADENYDAEQEPFKNTLVRHQDRFPYFALRYFDYNEIFTNLRFQIDLGTYHF |
| | SIYKKLIGGQKEDRHLTHKLYGFERIQEFAKQNRPDEWKAIVKDSDTFKKKEELKGKTIGVQLGSIQEQFAK |
| | DNGSVPKLYNNFTEALLDLQNQKIDAVIIAEVSGNEYLKTMKGIKKIDTIKDKLPSASIAFRKADSKLTKEFS |
| | DAILKLKDSQKISATVEIAAIFSEKTTELNSSRCLIP |
| UPJU01.1 | VKMKEEEEKGKTPVVSTYNKDDKHFWAAFLNLARHNVYITINHINKLLEIREIDNDEKVLDIKALWQKVN |
| SEQ ID NO: | KDLNQKARLRELMTKHFPFLETAIYTKNKEDKKEVKEEKQAKAQSFDSLNHCLFLFLEKLQEARNYYSHY |
| 4315 | KYSESSKEPMLEKELLKKMYNIFDNNIQLVIKDYQHNKDINPDEDFKHLDRTEEDFNYYFARNKKGNITAS |
| | GLLFFVSLFLEKKDAIWMQQKLTGFKDNRENKKKMTHEVFCRSRMLLPKLRLESTQTQDWILLDMLNELI |
| | RCPKSLYERLKGEDRKKFEVPFDSTDEDYDAEQDPFKNTLIRHQDRFPYFVLRYFDYNEIFKNLRFQIDLGT |
| | YHFSIYKKLIGGQKEDRHLTHKLYGFERIQEFTKQHRPDDWKAIVKDFDTYETSEEPYISETTPHYHLENQKI |
| | GIRFRNGNNDIWPSLETNGENNEKSKYKLDKPYQAEAFLSVHELLPMMFYYLLLKKEEPNNDKKNASRVE |
| | GFIKREIRDIFKLYDAFANDEINNIDDLKKYCKDKHIEIRHLPKQMIAILESKPKDMAKEAKRKQKEMVKDT |
| | KKLLATLEKQTQKEKEDDGRNVKLLKSGEIARWLVNDMMRFQPVQKDNEGKPLNNSKANSTEYQMLQR |
| | SLALYNKEENPTRYFRQVNLIESSNPHPFLKWTKWEECNNILTFYYTYLTKKIEFLNKLKPEDWKKNQYFL |
| | KLKEPKTNRKTLVQGWKNGFNLPRGIFTEPIREWFERHQNDSEEYKKVEKLNKAGLVTKVIPL |
| UPKI01.1 | VKMKEEETPVVSTYNKDDKHFWAAFLNLARHNVYITINHINKLLEIREIDNDEKILDIKTLWEKVNGDLNK |
| SEQ ID NO: | TERLRELMTKHFPFLETAIYSKNKEDKEEVKQEKQATAQSFKSLEHCLFLFLKKLQEARNYYSHYKYSEST |
| 4316 | KEPMLEKELLKKMYNIFDDNIQLVIKDYQHNKDINPDEDFKHLNRTEKEFNYYFTTNKKGNITASGLLFFVS |
| | LFLEKKDAIWMQQKLRGFKDNRESKKKMTHEVFCRSRMLLPKLRLESTQTQDWILLDMLNELIRCPKSLY |
| | ERLQGVDREKFRVPIEIADEDYDAEQEPFKNTLVRHQDRFPYFALRYFDYNEIFTNLRFQIDLGTYHFSIYKK |
| | LIGGQKEDRHLTHKLYGFERIQEFNKQNRPDEWKALVKDLDTYETSEEPYISETTPHYHLENQKIGIRFRNG |
| | NNDIWPSLETNGENNEKSKYKLDKQYQAEAFLSVHELLPMMFYYLLLKKEKPNNDEINASIVEGFIKREIR |
| | YIYKLYDAFANGEINSIGDLEKYCEDKGIPKRHLPKQMVAILYDEHKDMVKEAKRKQRKMVKETEKLLAA |
| | LEKQTQEEIEDGGRNIRLLKSGEIARWLVNDMMRFQPVQKDNEGNPLNNSKANSTEYQMLQRSLALYNKE |
| | EKPTRYF |
| UPIM01.1 | MEDDKKTTGSISYELKDKHFWAAFLNLARHNVYITINHINKLLEIREIDNDEKVLDIKALWEKVNGDLNKT |
| SEQ ID NO: | ERLRELMTKHFPFLETAIYTKNKEDKKEVKQEKQAEAQSLESLKDCLFLFLEKLQEARNYYSHYKYSEFSK |
| 4317 | EPEFEEGLLEKMYNIFDDNIQLVIKDYQHNKDINPDEDFKHLDRKGQFKYSFADNEGNITESGLLFFVSLFLE |
| | KKDAIWMQQKLRGFKDNRESKKKMTHEVFCRSRMLLPKLRLESTQTQDWILLDMLNELIRTNACKENTE |
| | KSSISPSTPQTKTTMQSKSLSKTHW |
| OGJT01.1 | LQKQDKLFVDRKKNAIFAFPKYITIMENKEKTEPIYYELTDKHFWAAFLNLARHNVYTTVNHINKLLEIAEL |
| SEQ ID NO: | KNDEDVLNIKDSWNKQAEKLDKKVRLRNLLMRHFPFLEAAAYEKTTSKDSNSKEQKEKEQAEALSLDNL |
| 4318 | KNVLFIFLEKLQSLRNYYSHYKYSEEAQKPTFENDLLKNMYKIFDTNVRLVKRDYMHHENVDMQRDFSHL |
| | NRKKQEGQTRKIIANPNFRYHFADEKGNMTIAGLLFFISLFLDKKDAIWMQKKLKGFKDGRNLREQMTNE |
| | VFCRSRISLPKLKLENVQTKDWMQLDMLNELVRCPKSLYERLREKDRESFKVPFDIFSDDYDAEEEPFKNT |
| | LVRHQDRFPYFVLRYFDLNEIFEQLRFQIDLGTYHFSIYNKLIGDEDEVRHLTHHLYGFARIQDFAPQNQPEE |
| | WRKLVKDLDHFETSQKPYISKTAPHYHLENEKIGIKFCSTHNNLFPSLKREKTCNGRSKFNLGTQFTAEAFL |
| | SVHELLPMMFYYLLLTKDYSRKESADKVEGIIRKEISNIYAIYDAFANGEINSIADLTCRLQKTNILQGHLPK |
| | QMISILEGRQKDMEKEAERKIGEMIDDTQRRLDLLCKQTNQKIRIGKRNAGLLKSGKIADWLVNDMMRFQ |
| | PVQKDQNNIPINNSKANSTEYRMLQRALALFGSENFRLKAYFNQMNLVGNDNPHPFLAETQWEHQTNILSF |
| | YRNYLEARKKYLKGLKPQNWKQYQHFLILKVQKTNRNTLVTGWKNSFNLPRGIFTQPIREWFEKHNNSKR |
| | IYDQILSFDRVGFVAKAIPLYFAEEYKDNVQPFYDYPFNIGNRLKPKKGNS |
| GCA_ | MRTIRTVRKKTPSKTRWSGIRIASPTSRYFDLKKVFTSLRFHIDLGTYHFAIYKKNIGEQPEDRHLTRNLYGF |
| 000503975.1_ | GRIQDFAEEHRPEEWKRLVRDLDYFETGDKPYITQTTPHYHIEKGKIGLRFVPEGQHLWPSPEVGATRTGRS |
| SJD2_genomic | KYAQDKRFTAEAFLSVHELMPMMFYYFLLREKYSDEASAERVQGRIKRVIEDVYAVYDAFARGEIDTLDR |
| SEQ ID NO: | LDACLADKGIRRGHLPRQMIAILSQEHKDMEEKVRKKLQEMIADTDHRLDMLDRQTDRKIRIGRKNAGLP |
| 4319 | KSGVIADWLVRDMMRFQPVAKDTSGKPLNNSKANSTEYRMLQRALALFGGEKERLTPYFRQMNLTGGNN |
| | PHPFLHETRWESHTNILSFYRSYLKARKAFLQSIGRSDRVENHRFLLLKEPKTDRQTLVAGWKGEFHLPRGI |
| | FTEAVRDCLIEMGLDEVGSYKEVGFMAKAVPLYFERASKDRVQPFYGYPFNVGNSLKPKKGRFLSKEKRA |
| | EEWESGKERFRDLEAWSHSAARRIEDAFVGIEYASWENKTKIEQLLQDLSLWEAFESKLKVKADKINIAKL |
| | KKEILEAKEHPYLDFKSWQKFERELRLVKNQDIITWMMCRDLMEENKVEGLDTGTLYLKDIRTDVYEQGS |
| | LNVLNRVKPMRLPVVVYRADSRGHVHKEQAPLATVYIEERDTKLLKQGNFKSFVKDRRLNGLFSFVDTGG |
| | LAMEQYPISKLRVEYELAKYQTARVCAFEQTLELEESLLTRYPHLPDKNFRKMLESWSDPLLDKWPDLHR |
| | KVRLLIAVRNAFSHNQYPMYDEAVFSSIRKYDPSSLDAIEERMGLNIAHRLSEEVKQAKEMVERIIQV |
| GCA_ | MRTIRTVRKKTPSKTRWSGIRIASPTSRYFDLKKVFTSLRFHIDLGTYHFAIYKKNIGEQPEDRHLTRNLYGF |
| 002206065.1_ | GRIQDFAEEHRPEEWKRLVRDLDYFETGDKPYITQTTPHYHIEKGKIGLRFVPEGQHLWPSPEVGATRTGRS |
| SJD5_genomic | KYAQDKRFTAEAFLSVHELMPMMFYYFLLREKYSDEASAERVQGRIKRVIEDVYAVYDAFARGEIDTLDR |
| SEQ ID NO: | LDACLADKGIRRGHLPRQMIAILSQEHKDMEEKVRKKLQEMIADTDHRLDMLDRQTDRKIRIGRKNAGLP |
| 4320 | KSGVIADWLVRDMMRFQPVAKDTSGKPLNNSKANSTEYRMLQRALALFGGEKERLTPYFRQMNLTGGNN |
| | PHPFLHETRWESHTNILSFYRSYLKARKAFLQSIGRSDRVENHRFLLLKEPKTDRQTLVAGWKGEFHLPRGI |
| | FTEAVRDCLIEMGLDEVGSYKEVGFMAKAVPLYFERASKDRVQPFYGYPFNVGNSLKPKKGRFLSKEKRA |
| | EEWESGKERFRDLEAWSHSAARRIEDAFVGIEYASWENKTKIEQLLQDLSLWEAFESKLKVKADKINIAKL |
| | KKEILEAKEHPYLDFKSWQKFERELRLVKNQDIITWMMCRDLMEENKVEGLDTGTLYLKDIRTDVYEQGS |
| | LNVLNRVKPMRLPVVVYRADSRGHVHKEQAPLATVYIEERDTKLLKQGNFKSFVKDRRLNGLFSFVDTGG |
| | LAMEQYPISKLRVEYELAKYQTARVCAFEQTLELEESLLTRYPHLPDKNFRKMLESWSDPLLDKWPDLHR |
| | KVRLLIAVRNAFSHNQYPMYDEAVFSSIRKYDPSSLDAIEERMGLNIAHRLSEEVKQAKEMVERIIQV |
| IMG_ | MQKKIRGFKGGTETYQQMTNEVFCRSRISLPKLKLESLRTDDWMLLDMLNELVRCPKSLYDRLHEEDRAR |
| 3300007499 | FRVPVDILSDEDDTDGTEEDPFKNTLVRHQDRFPYFALRYFDLKKVFTSLRFHIDLGTYHFAIYKKNIGEQPE |
| SEQ ID NO: | DRHLTRNLYGFGRIQDFAEEHRPEEWKRLVRDLDYFETGDKPYITQTTPHYHIEKGKIGLRFVPEGQHLWP |
| 4321 | SPEVGATRTGRSKYAQDKRLTAEAFLSVHELMPMMFYYFLLREKYSDEASAERVQGRIKRVIEDVYAVYD |
| | AFARDEINTRDELDACLADKGIRRGHLPRQMIAILSQEHKDMEEKIRKKLQEMIADTDHRLDMLDRQTDRK |
| | IRIGRKNAGLPKSGVIADWLVRDMMRFQPVAKDTSGKPLNNSKANSTEYRMLQRALALFGGEKERLTPYF |
| | RQMNLTGGNNPHPFLHETRWESHTNILSFYRSYLKARKAFLQSIGRSDRVENHRFLLLKEPKTDRQTLVAG |
| | WKSEFHLPRGIFTEAVRDCLIEMHDEVGSYKEVGFMAKAVPLYFERACKDRVQPFYDYPFNVGNSLKPKK |
| | GRFLSKEDRAEEWESGKERFRLAKLKKEILEAKEHPYLDFKSWQKFERELRLVKNQDIITWMMCRDLMEE |
| | NKVEGLDTGTLYLKDIRTEVQEQGSLNVLNRVKSMRLPVVVYRADSRGHVHKEQAPLATVYIEERDTKLL |
| | KQGNFKSFVKDRRLNGLFSFVDTGGLAMEQYPISKLRVEYELAKYQTARVCAFEQMLELEESLLTRYPHLP |
| | DKNFRKMLESWSDPLLDKWPDLHRKVRLLIAVRNAFSHNQYPMYDEAVFSSIRKYDPSSLDAIEERMGLNI |
| | AHRLSEEVKQAKEMVERIIQV |
| UZOZ01.1 | VPFDIFSDDYNAEEEPFKNTLVRHQDRFPYFVLRYFDLNEIFTQLRFQIDLGTYHFSIYNKRIGDEDEVRHLT |
| SEQ ID NO: | HHLYGFARIQDFAQQNQPEVWRKLVKDLDYFEASQEPYISKTTPHYHLENEKIGIKFCSAHNNLFPSLQTDK |
| 4322 | TCNGRSKFNLGTQFTAEVFLSVHELLPMMFYYLLLTKDYSRKESANKVEGIIRKEISNIYDIYDAFANGEINS |
| | IADLTCRLQKTNILQGHLPKQMISILEGRQKDMEKEAERKIGEMIDDTQRRLDLLCKQTNQKIRIGKRNAGL |
| | LKSGKIADWLVSDMMRFQPVQKDTNNAPINNSKANSTEYRMLQHALALFGSESSRLKAYFRQMNLVGNA |
| | NPHPFLAETQWEHQNNILSFYRKYLEARKKYLGSLKPKDWKQYQHFLMLKEQKSNRNTLVAGWKNGFNL |
| | PRGIFTEPIRKWFEEHNNSEGLYDQILSFGRVGFVAKAIPLYFAEECKDCVQPFYDYPFNVGNKLKPKKGQF |
| | LDKKEHVELWQKNKELFKNYPPEKRKTDLAYLDFLSWKKFERELRLIKNQDIVTWLMFKELFKTTTVEGL |
| | KIGEIHLRDIDTNTANEESNNILNRIMPMKLPVKTYETDNKGNILKERPLATFYIEETETKVLKQGNFKVLAK |
| | DRRLNGLLSFAETTDIDLEKNPITKLSVDHELIKYQTTRISIFEMTLGLEKKLIDKYSTLPTDSFRNMLERWL |
| | QCKANRPELKNYVNSLIAVRNAFSHNQYPMYDATLFAEVKKFTLFPSVDTKKIELNIAPQLLEIVGKAIKEIE |
| | KSENKN |
| IMG_ | MQKKIKGFKGGTENYMRMTNEVFCRNRMVIPKLRLETDYDNHQLMFDMLNELVRCPLSLYKRLKQEDQ |
| 3300014026 | DKFRVPIEFLDEDNEADNSYQENANSDENPTEETDPLKNTLVRHQHRFPYFVLRYFDLNEVFKQLRFQINLG |
| SEQ ID NO: | CYHFSIYDKTIGERTEKCHLTRTLFGFDRLQNFSVKLQPEHWKNMVKHLDTEESSDKPYLSDAMPHYQIEN |
| 4323 | EKIGIHFLKTDTEKKETVWPSLEVEEVSSNRNKYKSEKNLTVDAFLSTHELLPMMFYYQLLSSEEKTRAAA |
| | GDKVQGVLQSYRKKIFDIYDDFANGTINSMQKLDERLAKDNLLRGNMPQQMLAILERQEXXXXICS |
| IMG_ | MQKKIPGFKKASENYMKMTNEVFCRNHILLPKIRLETVYDKDWMLLDMLNEVVRCPLSLYKRLTPADQN |
| 3300006479 | KFKVPEKSSDNANRQEDDNPFSRILVRHQNRFPYFVLRFFDLNEVFTTLRFQINLGCYHFAICKKQIGDKKE |
| SEQ ID NO: | VHHLTRTLYGFSRLQNFTQNTRPEEWNTLVKTTEPSSGNDGKTVQGVPLPYISYTIPHYQIENEKIGIKIFDG |
| 4324 | DTAVDTDIWPSVSTEKQLNKPDKYTLTPGFKADVFLSVHELLPMMFYYQLLLCEGMLKTDAGNAVEKVLI |
| | DTRNAIFNLYDAFVQEKINTITDLENYLQDKPILIGHLPKQMIDLLKGHQRDMLKAAEQKKAMLIKDTERR |
| | LERLNKQPEQKPNVAAKNIGALLRNGQIADWLVKDMMRFQPVKRDKEGNPINCSKANSTEYQMLQRAFA |
| | FYATDSCRLPRYFEQLHLINCDNSHLFLSRFEYDKQPNLIAFYAAYLKAKLDFLNELQPQNWVSDNYFLLL |
| | RAPKNNRQKLAEGWKNGFNLPRGLFTEKIKTWFNEHKTIVDISDCDIFKNRVGQVARLIPVFFDKKFKDHS |
| | QPFYRYNFNVGNVSKPTEAKYLSKEKREELFKSYQNKFKNNIPAEKTKEYREYKNFSLWKKFERELRLIKN |
| | QDILTWLMCKNLFDEKIEQGIDIPYIKLDSLQTNTSTKGSLNALAQVLPMVLAIYIGNSESNNGTGANEEEN |
| | KGPMVYIKEEGTKLLKWGNFKTLLADRRIKGLFSYIEHDDIDLKQHPLTKRRVDLELDLYQTCRIDIFQQTL |
| | GLEAQLLNKYSDLNTDNFYQMLIGWRKKEGIPRDIKEDTDFLKDVRNAFSHNQYPDSKKIAFSRIRKFNPK |
| | KTILNEKKGLGIAKQMYEEVEKVVNRIKGIELFD |
| GCA_ | MEKPLPPNVYTLKHKFFWGAFLNIARHNAFITICHINEQLGLTTPPNDDKIADVVCGTWNNILNNDHDLLK |
| 002025185.1_ | KSQLTELILKHFPFLAAMCYHPPKKEGKKKGSQKEQQKEKENEAQSQAEALNPSELIKALKTLVKQLRTLR |
| ASM202518v1_ | NYYSHYKHKKPDAEKDIFKHLYKAFDASLRMVKEDYKAHFTVNLTQDFAHLNRKGKNKQDNPKFDRYR |
| genomic_2 | FEKDGFFTESGLLFFTNLFLDKHDAYWMLKKVSGFKASHKQSEKMTTEVFCRSRILLPKLRLESRYDHNQ |
| SEQ ID NO: | MLLDMLSELSRCPKLLYEKLSEKDKKHFQVEADGFLDEIEEEQNPFKDALIRHQDRFPYFALRYLDLNESFK |
| 4325 | SIRFQVDLGTYHYCIYDKKIGDEQEKRHLTRTLLSFGRLQDFTEINRPQEWKALTKDLDYKETSKQPFISKTT |
| | PHYHITDNKIGFRLGTSKELYPSLEVKDGANRIAKYPYNSDFVAHAFISVHELLPLMFYQHLTGKSEDLLKE |
| | TVRHIQRIYKDFEEERINTIEDLEKANQGRLPLGAFPKQMLGLLQNKQPDLSEKAKIKIEKLIAETKLLSHRL |
| | NTKLKSSPKLGKRREKLIKTGVLADWLVKDFMRFQPVAYDVQNQPIESSKANSTEFQLIQRALALYGGEKN |
| | RLEGYFKQTNLIGNTNPHPFLNKFNWKACRNLVDFYQQYLEQREKFLEAIKNQPWEPYQYCLLLKIPKENK |
| | KIWLKVGNKEALACQEGCLPKRLERSFRKTSRYPNLLERRLKNTVEWALSPEPLHCTLGKDTKTTTRVFTT |
| | FRTS |
| GCA_ | MEKPLPPNVYTLKHKFFWGAFLNIARHNAFITICHINEQLGLKIPSNDDKIADVVCGTWNNILNNDHDLLKK |
| 001670645.1_ | SQLTELILKHFPFLAAMCYHPPKKEGKKKGSDKEQQKEKENEAQSDAEALNPSELIKALETLVNQLHNLRN |
| RCAD0181_ | YYSHYKHKKPDAEKDIFKHLYKAFDASLRMVKEDYKAHFTVNLTRDFAHLNRKGKNKQDNPKFDRYRFE |
| genomic_2 | KDGFFTESGLLFFTNLFLDKRDAYWMLKKVSGFKASHKQSEKMTTEVFCRSRILLPKLRLESRYDHNQML |
| SEQ ID NO: | LDMLSELSRCPKLLYEKLSEENKKHFQVEADGFLDEIEEEQNPFKDALIRHQDRFPYFALRYLDLNESFKSIN |
| 4326 | SLCILNNTDF |
| GCA_ | MEKPLPPNVYTLKHKFFWGAFLNIARHNAFITICHINEQLGLKIPSNDDKIADVVCGTWNNILNNDHDLLKK |
| 001670765.1_ | SQLTELILKHFPFLAAMCYHPPKKEGKKKGSQKEQQKEKENEAQSQAEALNPSELIKALETLVNQLHNLRN |
| RCAD0133_ | YYSHYKHKKPDAEKDIFKHLYKAFDASLRMVKEDYKAHFTVNLTRDFAHLNRKGKNKQDNPKFDRYRFE |
| genomic_2 | KDGFFTESGLLFFTNLFLDKRDAYWMLKKVSGFKASHKQSEKMTTEVFCRSRILLPKLRLESRYDHNQML |
| SEQ ID NO: | LDMLSELSRCPKLLYEKLSEENKKHFQVEADGFLDEIEEEQNPFKDALIRHQDRFPYFALRYLDLNESFKSIN |
| 4327 | SLCILNNTDF |
| GCA_ | LPRGLFTEAIREILSEDLTLSKPIRKEIKKHGRVGFISRAITLYFRERYQDDHQSFYNLPYELEAKASTPKPPLP |
| 002025185.1_ | KKREYVLRAEHYEYWQQNKPQSPTELQRLELHTSDRWKDYLLYKRWQHLEKKLRLYRNQDVMLWLMT |
| ASM202518v1_ | LELTKNHFKELKLNYHQLKLENLAVNVQEADAKLNPLNQTLPMVLPVKVYPATAFGEVQYQETPIRTVYI |
| genomic | REEHTKALKMGNFKALVKDRRLNGLFSFIKEENDTQKHPISQLRLRRELEIYQSLRVDAFKETLDLEEKLLK |
| SEQ ID NO: | KHTSLSSLENKFRILLEEWKKEYAASSMITDEHIAFIASVRNAFCHNQYPFYEEALHAPIPLFTVAQPTTEEK |
| 4328 | DGLGIAEALLRVLREYCEIVKSQI |
| GCA_ | SIRFQVDLGTYHYCIYDKKIGDEQEKRHLTRTLLSFGRLQDFTEINRPQEWKALTKDLDYKETSKQPFISKTT |
| 001670645.1_ | PHYHITDNKIGFRLGTSKELYPSLEVKDGANRIAKYPYNSDFVAHAFISVHELLPLMFYQHLTGKSEDLLKE |
| RCAD0181_ | TVRHIQRIYKDFEEERINTIEDLEKANQGRLPLGAFPKQMLGLLQNKQPDLSEKAKIKIEKLIAETKLLSHRL |
| genomic | NTKLKSSPKLGKRREKLIKTGVLADWLVKDFMRFQPVAYDVQNQPIESSKANSTEFQLIQRALALYGGEKN |
| SEQ ID NO: | RLEGYFKQTNLIGNTNPHPFLNKFNWKACRNLVDFYQQYLEQREKFLEAIKNQPWEPYQYCLLLKIPKENR |
| 4329 | KNLVKGWEQGGISLPRGLFTEAIRETLSEDLTLSKPIRKEIKKHGRVGFISRAITLYFRERYQDDHQSFYNLP |
| | YELEAKASTPKPPLPKKREYVLRAEHYEYWQQNKPQSPTELQRLELHTSDRWKDYLLYKRWQHLEKKLR |
| | LYRNQDVMLWLMTLELTKNHFKELKLNYHQLKLENLAVNVQEADAKLNPLNQTLPMVLPVKVYPATAF |
| | GEVQYQETPIRTVYIREEQTKALKMGNFKALVKDRRLNGLFSFIKEENDTQKHPISQLRLRRELEIYQSLRV |
| | DAFKETLSLEEKLLNKHASLSSLENEFRTLLEEWKKKYAASSMVTDEHIAFIASVRNAFCHNQYPFYKETL |
| | HAPILLFTVAQPTTEEKDGLGIAEALLRVLREYCEIVKSQI |
| GCA_ | SIRFQVDLGTYHYCIYDKKIGDEQEKRHLTRTLLSFGRLQDFTEINRPQEWKALTKDLDYKETSKQPFISKTT |
| 001670765.1_ | PHYHITDNKIGFRLGTSKELYPSLEVKDGANRIAKYPYNSDFVAHAFISVHELLPLMFYQHLTGKSEDLLKE |
| RCAD0133_ | TVRHIQRIYKDFEEERINTIEDLEKANQGRLPLGAFPKQMLGLLQNKQPDLSEKAKIKIEKLIAETKLLSHRL |
| genomic | NTKLKSSPKLGKRREKLIKTGVLADWLVKDFMRFQPVAYDVQNQPIESSKANSTEFQLIQRALALYGGEKN |
| SEQ ID NO: | RLEGYFKQTNLIGNTNPHPFLNKFNWKACRNLVDFYQQYLEQREKFLEAIKNQPWEPYQYCLLLKIPKENR |
| 4330 | KNLVKGWEQGGISLPRGLFTEAIRETLSEDLTLSKPIRKEIKKHGRVGFISRAITLYFRERYQDDHQSFYNLP |
| | YELEAKASTPKPPLPKKREYVLRAEHYEYWQQNKPQSPTELQRLELHTSDRWKDYLLYKRWQHLEKKLR |
| | LYRNQDVMLWLMTLELTKNHFKELKLNYHQLKLENLAVNVQEADAKLNPLNQTLPMVLPVKVYPATAF |
| | GEVQYQETPIRTVYIREEQTKALKMGNFKALVKDRRLNGLFSFIKEENDTQKHPISQLRLRRELEIYQSLRV |
| | DAFKETLSLEEKLLNKHASLSSLENEFRTLLEEWKKKYAASSMVTDEHIAFIASVRNAFCHNQYPFYKETL |
| | HAPILLFTVAQPTTEEKDGLGIAEALLRVLREYCEIVKSQI |
| IMG_ | MNNLINVNFKKFDLESDKYFFAAYLNKATQNVYIILKDISESLGLGFELLNDNNIMTANMWSYLQSNKEPE |
| 3300025308_3 | VSIRIIEKLNKQFPFINYLAKRNSFVKRNEFVATPYDYYEVFELIIEQLVKFRNYYSHPLSEKVVMKQSIIEGM |
| SEQ ID NO: | RVLFDSSRREVKKRFAFKTKEINHLVRLSSRKINDKKESYESEFFEYRFADKFNLISEKGFAFFVSMWLPRTD |
| 4331 | AQFFLKNIKGFSRTDTISQKAILEIFTFYSLRIPQTKFECDYSNEIYFSEMINELQRCPKELYHLLNQEDREKFE |
| | SLNNSRFNNNEYEALPILKRNDNRFFYFALRYLDNIFKNTKFNIDLGYYCFHVYNQEIDGIARKRRWVKHIS |
| | VFGNLKDFTLNNRPKEWIEKINKNQDSQNENNEIYVTETVPHYHINEHNIGLKIINNYSDLKNSNKIWPELQ |
| | KFKFENKEKLKPRNEKPDCWLSLYELPAIVFYEILRRKNNKGDSAEKIINNHCNNIKSFFEDIEKGLFHSNYT |
| | EDALKKELENRNLEKSHIPKAIIKYLTSAKTESFEVKAQNKLDELYYETCEMLNKINRQKSSFCEKPGSKDY |
| | VEMKCEVLADFLARDMIRLQKPIRNDFGKANGTEFSLLHAKLAFFGINKNTLLDTFKLCNLSESSNPHPFLD |
| | KINIKQSNNLLDFYEIYLNNRKDYFKSCLTEKKYNEYHFLKLGERRKKSGEKYIIKLAKELKDENVINLPRG |
| | LFLNPIIETLLIDDKTKSLAQEVKKMKRVNVSFIIEKYFSEIRKDQPQEFYRYKKSYEILNKLFDNREKFEKRE |
| | ALTKKQFDTNELEKLTTKIYEKTSDNELTRLLAQKIKSDI |
| MWWF01.1 | LFMPFYHSTIDKALFGAYLNMARNNLRMVLTNIEEKVYGKSGNWKEDNMRHSPVIKALEKNEQPDISKRIT |
| SEQ ID NO: | DMLLRDLPFLKLTVKSKINPSPNDYAKSLIGFIEQLSDLRNFNCHYLHNSYKTNPEIIESLRHIFDAARRRVKI |
| 4332 | RFNNTTEEVEHLVRKTARMVNGKRVGIEKKNYSFAFADAHEEITEIGIAFFTCLFLDKKYITLFLKQLKGFK |
| | DSRTRKTRATINTFGFFNIRIPQPKLQSNDTKEGLLMDILEDLKRCPNELFEHLSAKDQERFRVKADNLIEEN |
| | EVLQKRFSDRFPYLALRYFELTDHVKDFQFHTDLGKYNFKHYKKQVGGEERIRQLQKNMKGTGRLKDFTE |
| | DKMPEEWNVLVKRSDELPEGYMEPHIVFTIPHYHFVNNQIGVCINDVAKYPDAKTRKNPEPDIWLSIYELP |
| | ALIFLHLLTKRDDGSSEIKWVLYRHDQNIKRFFKALVEDKLQPGFTKETLQSELNKYNLEYSHIPKVLIEYLL |
| | KKTVKNVQQKAEDKLHVLLSETIVLLRKFDEDVEKAKEKEGGKRYKPIQSGKIADFIARDMLFLQPPVNEK |
| | DGKANSTLFQVLQARLAYYGRDKNLLNSLYKECNLIDSANAHPFLNNVPLAYSIVDYYKNYLLKRKEYLE |
| | KLLEKCMKGKINSNEFHFLHLGAREKRSGDSYYKNLAKTFSEKPINFPRGLFLNAIKQQSKNKNGESIKKFL |
| | EEHERVNTIFLIQEHFKTVEEDEAQPF |
| IMG_ | MDTPSSIERKHIVLTDKYYFAAYLNMARHNVYMVLTDINTRLGFEKVPGDDAGAVSVIVLQKLKEDSTKK |
| 3300030508 | NVIAPDIQLKIIKELHAHFSFLKPMMFAWKKPIGEATEEEIQKMEYAPGDYYTFFAVFLKALNDLRNEYTHV |
| SEQ ID NO: | ATQPFDFPADLLHALRVTFDAGVRKAKARFSFEAKDVEHLVRQVKGGKEKPAFKYKFQLKGSKSLTTYGL |
| 4333 | SFFICLLLERKYASLFTQKLEGLKDKRTRAFKATYEVFAVHCITLPKARYTSDSGEGSLLLDMLNELKRCPD |
| | ILFPHLQAKNQDVFRIPVEDVPGMEQEEDNNFVLLKRYEDRFPYLALRFMDEVKWFQKLHFPVDLGNYHY |
| | HLYDKTVDGMPRVGSLWEKMIGYGRLPDYQQAFLEKKVPDSWLRLWKNHEVRTEGAKAPYIPPAMPHY |
| | HLPDNNIAIRITTENGWPDLTINEADADKNKPGKKH |
| IMG_ | MNNEQNLEQRIFAQAKNIRDDKAYFGAHLNLARHNAFIILHHINQRLGFIDQNVQDDAQFKKFKCLTILKQ |
| 3300007465 | SSKPDLIAKSLDLIRFHFPFLQILTDKLSDIRSSNGERKILTPQEEGEIIESLLTDLNGYRNEYCHGENKSHVPD |
| SEQ ID NO: | SYLIKNLKSIYDASLRMVKERFKLEPHKIKHLERNNKDGEKLNFKYALSNNSSISEKGLVFFINLFLEVKDSY |
| 4334 | SFIKKIEGFKNAGTNSLNATTYCFTIQHVRLPNPKITSQEYTKEELLLRMMNYLEKVPDQIFKTLSPADQENC |
| | KSNVDVFETPIEGDLIEKSLHKRYRDQFPEFALNYIDYYKLFPNIRFQINLGKFIFSVKDKEILGETRKRRQIQ |
| | MLRGFGQIQDYQNKKNIPEIWQSLINKSHEIPEDFPDPYINDMEPHYHIENNNIYFKFMEPDTTHWPRIDARI |
| | ENQNNINAHPRPRLLQADGILSVYELAPLLFYEMIRSDKSKSAETILSIQKSNIERLLNDIKSGELTKVALDKI |
| | PNPYSPSTKKFNTAKQVILKEKIEKRKIELNEKLKKYKLALDDLPKMILYYLLNIEHEDISLKVIPIIKSITVIL |
| | NTQTHPANFEYLAVPKKSKHAYLKAMITNWEELNISTGPMGIYFANTFVGTTTLNPESIEDTLSISLGPDIAT |
| | QLKRTKIAENTKKETFSAKKHSNIAWEIDIKNSKTRDIEVRIEDQIPLSKLNEVEVETKELSGGMLDQSTGIIT |
| | WNVKIPAGKSIKKILKYQVRYPKSMKLILE |
| IMG_ | METEEQIIENRIRTLANDPQYFGGYLNMARHNIYLIINNLTKTFSYLNFKEIADDAEIASDTHILSNIFDTSNSII |
| 3300028769 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4335 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300028862_3 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4336 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300028767_5 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4337 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300028774_2 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4338 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300028738 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4339 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300028739 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4340 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300029998_3 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4341 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300030055_2 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4342 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300028864 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4343 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300030047_2 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4344 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300030491_2 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4345 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300030048_2 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4346 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300030001_2 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4347 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300031918_2 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4348 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300030000 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4349 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300030002 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4350 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300030673_2 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4351 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300029981_2 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4352 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300030943_2 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4353 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300030685_2 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4354 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300028853 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4355 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300029995_2 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4356 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300030294 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4357 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300029923_3 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4358 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300029989 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4359 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300030339_2 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4360 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300028676_2 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4361 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300029983_3 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4362 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300030019_2 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4363 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300029990_2 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4364 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300028772 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4365 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300030838_2 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4366 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300031521_4 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4367 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300031722 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4368 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300028763 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4369 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300030230 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4370 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METEEQIIENRIRTLANDPQYFGGYLNMARHNIYLIINNLTKTFSYLNFKEIADDAEIASDTHILSNIFDTSNSII |
| 3300028734_2 | DEERIKVYNYLIKRHYLPFLKIFNAENIIEIGNEYTIDFKRLHNFIIKSFKKITDLRNAYSHYLSIDDDGNIANS |
| SEQ ID NO: | NKKELDSSIKGDIDLLFKYAPQYSYIRNNQTQTGTDYTHLENYLLFEISENNTLTDQGLYFFINLFLTREHAT |
| 4371 | KFLKRFKGFKNETTPPFRATIQAFTSYALKLPDERLGNENPIHSLLMEILAELNKCPKELFIHLTDEWKKEFE |
| | PVLSENGRKNIVLNSINYNELNNEDIEEVTKELSTLKRYDDRYPYFALRYLEDTNCLKRIRFQITLGKLIVNR |
| | YDKKIIGINQDRRVLKTVNTFGKLSDFVDKESDVLEILKHHVINTENIVFEQYAPHYNTNNNKIAFYIFDEED |
| | EKMRR |
| IMG_ | METKEQIGKNVYKTSENDPLYFGGYLNMARHNVFLIINHLTEVFDSLGYTKINDDEDIVNQDHILSQIFDPS |
| 3300023244 | KKELENERIRIYNYMIKRHHLPFLKVFNSEILNDEDGENMGIDFKSLHNFIIKSFKTLNDLRNSYSHYLAIDD |
| SEQ ID NO: | DGNKIEKRSNIVDVSIKSDIKQLFKHAPKFSFIRNQETQHEEDYNHLDRYRIFENETNILTDQGLYFFINLFLE |
| 4372 | RNHATKFLKKIKGFKNETTPPFRATIQSFTSFALKLPDIRLSNERPLFSLLMNMLTELNKCPKELFNHLTQKD |
| | KKEFEPLLNDEEKHNVVLNSTNYSEISDDELDEAIREITALKRYNDRFPYFALRFLDETNALKNIRFQITLGK |
| | LIIKRYDKEIAGIEENRRVIKTINAFGKLSDFIDNEEVVLKELKKNLADNNDIRFEQYSPHYNTNNIKIAFYVF |
| | DEGDDKTKYPFVFENKENNSDIQNNPSGFLSIHDLPKMLLFECLDINLKPENIIIDFIRSTNLEMFDLSELEKIR |
| | KQANYEPEYFSKRINKEKYLISKKGIKYLSKEVENNMLEDLGLSKDELILKDKDSFMKLTNSKKYIEYFSQI |
| | KYQ |
| IMG_ | MEQNRKEGSLRTQEDIQYFGSYFNMARHNLYLITNHLTSVFSHLNFSQLDDDEDIWSDKPEVNEKNILLNIF |
| 3300007584 | DTKNERLQDERIRVFRYLMRRHHLPCLRIFTNDFKVLETTGNTIKKDELEVDFDAVHKFLNIAFKEINLFRN |
| SEQ ID NO: | SYTHYLSINEEGKRLKKKKKISSKLVPVLNSLFSYAPEYSFLRHNIDKANKDVKIYKEEVKEYYDNIKSKYK |
| 4373 | LFENDSNELTDQGMYFFITLFLERAHGIKFLKRFRGFKNETTPPFKATIQAFTTYTLKIPDVRLDNDIPEQTMI |
| | MEVLNELNKCPEELFKFLKKEDKDRFQPVISDESLTNIINSSNYEEISDEDIDRLIKENSVLKRREDRFPYFAL |
| | QYLEIMSKLKNIRFQIYLGKLVLKTYDKENPNIERRVITDIHAFGKLSDFVGKEAEVLDTFNSQLKDYGYSV |
| | AWDQYKPSYAIEMNRIGFYLFNDQGDKVENKILPSLCKNKNLERKVEIKVNKIQPTGFISTHMLPKFLISYL |
| | MGDVNEKNGEKTITHFLEKVNVSILDQNIINQIKSEIQNLDPIEFTKRCPKLSAIKNVKKLRVAGAIDKKIVE |
| | YQYINDTDIAKLVQKTGLAYDTMVTYSKDKFKEKTNHLNLSKKELETFAHIKYKYYLSERRKALDSVLKA |
| | YFPEIGSQDIPKELYNYLLNINEQDNKKLEHRRIKDEVTKTKKLIKDVNRSLKFEDKILLGDLATKIARD |
| IMG_ | MEQNRKEGSLRTQEDIQYFGSYFNMARHNLYLITNHLTSVFSHLNFSQLDDDEDIWSDKPEVNEKNILLNIF |
| 3300007483_2 | DTKNERLQDERIRVFRYLMRRHHLPCLRIFTNDFKVLETTGNTIKKDELEVDFDAVHKFLNIAFKEINLFRN |
| SEQ ID NO: | SYTHYLSINEEGKRLKKKKKISSKLVPVLNSLFSYAPEYSFLRHNIDKANKDVKIYKEEVKEYYDNIKSKYK |
| 4374 | LFENDSNELTDQGMYFFITLFLERAHGIKFLKRFRGFKNETTPPFKATIQAFTTYTLKIPDVRLDNDIPEQTMI |
| | MEVLNELNKCPEELFKFLKKEDKDRFQPVISDESLTNIINSSNYEEISDEDIDRLIKENSVLKRREDRFPYFAL |
| | QYLEIMSKLKNIRFQIYLGKLVLKTYDKENPNIERRVITDIHAFGKLSDFVGKEAEVLDTFNSQLKDYGYSV |
| | AWDQYKPSYAIEMNRIGFYLFNDQGDKVENKILPSLCKNKNLERKVEIKVNKIQPTGFISTHMLPKFLISYL |
| | MGDVNEKNGEKTITHFLEKVNVSILDQNIINQIKSEIQNLDPIEFTKRCPKLSAIKNVKKLRVAGAIDKKIVE |
| | YQYINDTDIAKLVQKTGLAYDTMVTYSKDKFKEKTNHLNLSKKELETFAHIKYKYYLSERRKALDSVLKA |
| | YFPEIGSQDIPKELYNYLLNINEQDNKKLEHRRIK |
| IMG_ | MEQNRKEGSLRTQEDIQYFGSYFNMARHNLYLITNHLTSVFSHLNFSQLDDDEDIWSDKPEVNEKNILLNIF |
| 3300007483 | DTKNERLQDERIRVFRYLMRRHHLPCLRIFTNDFKVLETTGNTIKKDELEVDFDAVHKFLNIAFKEINLFRN |
| SEQ ID NO: | SYTHYLSINEEGKRLKKKKKISSKLVPVLNSLFSYAPEYSFLRHNIDKANKDVKIYKEEVKEYYDNIKSKYK |
| 4375 | LFENDSNELTDQGMYFFITLFLERAHGIKFLKRFRGFKNETTPPFKATIQAFTTYTLKIPDVRLDNDIPEQTMI |
| | MEVLNELNKCPEELFKFLKKEDKDRFQPVISDESLTNIINSSNYEEISDEDIDRLIKENSVLKRREDRFPYFAL |
| | QYLEIMSKLKNIRFQIYLGKLVLKTYDKENPNIERRVITDIHAFGKLSDFVGKEAEVLDTFNSQLKDYGYSV |
| | AWDQYKPSYAIEMNRIGFYLFNDQGDKVENKILPSLCKNKNLERKVEIKVNKIQPTGFISTHMLPKFLISYL |
| | MGDVNEKNGEKTITHFLEKVNVSILDQNIINQIKSEIQNLDPIEFTKRCPKLSAIKNVKKLRVAGAIDKKIVE |
| | YQYINDTDIAKLVQKTGLAYDTMVTYSKDKFKEKTNHLNLSKKELETFAHIKYKYYLSERRKALDSVLKA |
| | YFPEIGSQDIPKELYNYLLNINEQDNKKLEHRRIKXXXXXXXXXXXXXXXXXXXXXXCKS |
| GCA_ | MEKNSLQDTTRTKDDVLYFGSYLNMGRHNVYLIINHVTEVFKHLGFRKLNDDEDIWSEKEQVNEGNILLNI |
| 003457245.1_ | FDPKKEKYQDERFRVFNYLIKRHHLPFLRIFTNQVLNDSGEIQNPEKKDMLIDFEGAHVFINKIFRELNEFRN |
| ASM345724v1_ | SYTHYLSLSNEGTPLPKKLQINVELIKDLKTLFYYAPEFSFIRHNVLKQESKEEYETKVKAYYNDIRRKYRLF |
| genomic | EGDESAGKLTDQGLFFFVNLFLERSNAIKFLKRFRGFKNETLPPFKATIQSFTTYALKIPDVRLDNDFPKQAL |
| SEQ ID NO: | LMEILTELNRCPKELYQVLGKEDKAKFDPKLEQSAINNILENVNYDELSDEHLEQAIKELVVLKRHDDRFPY |
| 4376 | FALRYLDEMNLLSQIRFQVYLGKVELKSYFKDDLGIERRILKPIYAFGKLSDFDNKEEDILRELKKNLPPDCQ |
| | DIHWDQYKPHYNISQNN |
| IMG_ | MDIIPKLTYTIESTPWYFGAYLNMARHNVYLLINHLTEKFSHLKYEKLKDDKEIKGKNILTEIFDTTKSDLDE |
| 3300022741 | ERFRIYKYLVRGHYLPFIKVYSDSKGNALENNPLVYYDRLHQFINNSFALLVKFRDAFSHYLALDEHGNSID |
| SEQ ID NO: | SRQLNIDHEIAHDLETIFQDSLSLSASRFYLTQQESDFEHLKHYALFKETETKLSENGFYFFICLFLEKQYAIK |
| 4377 | FLKKIKGFKNETIPAFRATLLAFTHYTIRIPDIRLDNDEPRMGVLMEMLNELQKCPIELYKRLTDEDKKKFEP |
| | ALDEESQLNLILNSTANSENLSDEQTDSLLIDLTTLKRHQNRFSYFALRCIDELNLLPGIHFQITVGKIELRAY |
| | PKVIGKVATNRRILKEVNAFGKLSAYEGKENWFSQQLKMIYEDENLVFDQYNPHYNIQENKIAFYVLDSGT |
| | SGTLLPLKKKNTLPTGFLSLNDLPKLIVRALNSPGRTVSLIKDFIAKNENIILNEDALVAWKEQLHLDPAVFT |
| | RRIIKENALRGKEGIAYLTQRKTDALFKRYKALSIKIDSLAGLKKLIDQLHSKKDKEYISQIVYTHFLNKRKD |
| | ALAGILPKGLPVNQLPLKVINYLLSLETVGHKKKFLHYIKEEKRTCKTRLKALNKQENNAPKIGEIATFLAR |
| | DIINMVVNEETKQNITSAYYNRLQNKIAYFSISKPEIAEMLTELNLFDKKTGHPFLDKGSIMASSGILAFYEY |
| | YLVEKAAWIDKQILHKNQLKKDLESHLNKLPFAYARRYQKNNEVN |
| IMG_ | MDIIPKLTYTIESTPWYFGAYLNMARHNVYLLINHLTEKFSHLKYEKLKDDKEIKGKNILTEIFDTTKSDLDE |
| 3300022741_2 | ERFRIYKYLVRGHYLPFIKVYSDSKGNALENNPLVYYDRLHQFINNSFALLVKFRDAFSHYLALDEHGNSID |
| SEQ ID NO: | SRQLNIDHEIAHDLETIFQDSLSLSASRFYLTQQESDFEHLKHYALFKETETKLSENGFYFFICLFLEKQYAIK |
| 4378 | FLKKIKGFKNETIPAFRATLLAFTHYTIRIPDIRLDNDEPRMGVLMEMLNELQKCPIELYKRLTDEDKKKFEP |
| | ALDEESQLNLILNSTANSENLSDEQTDSLLIDLTTLKRHQNRFSYFALRCIDELNLLPGIHFQITVGKIELRAY |
| | PKVIGKVATNRRILKEVNAFGKLSAYEGKENWFSQQLKMIYEDENLVFDQYNPHYNIQENKIAFYVLDSGT |
| | SGTLLPLKKKNTLPTGFLSLNDLPKLIVRALNSPGRTVSLIKDFIAKNENIILNEDALVAWKEQLHLDPAVFT |
| | RRIIKENALRGKEGIAYLTQRKTDALFKRYKALSIKIDSLAGLKKLIDQLHSKKDKEYISQIVYTHFLNKRKD |
| | ALAGILPKGLPVNQLPLKVINYLLSLETVGHKKKFLHYIKEEKRTCKTRLKALNKQENNAPKIGEIATFLAR |
| | DIINMVVNEETKQNITSAYYNRLQNKIAYFSISKPEIAEMLTELNLFDKKTGHPFLDKGSIMASSGILAFYEY |
| | YLVEKAAWIDKQILHKNQLKKDLESHLNKLPFAYARRYQKNNEVN |
| IMG_ | MEILTIPEVIKCRTLSDDPQYFGGYLNMARLNVLNISNHIAKEFKLPLLPEEAHLKNSFLCKKENKKIDWNH |
| 3300028769_3 | VYARTIRFLSVMKVFDAESLPKEEQKTIDWEGKDFASMCDTLNIVFSELQEFSNDYSHYYSTEKETKRKTT |
| SEQ ID NO: | VSDELALFLRTNFKRAIEYTKVRFKGILNDEDYQLVVSKKMLETNHTITHEGLVFLTSMFLEREYAFRFIRKI |
| 4379 | HGLSGPKDNSFIATCEVLMAFCLKLPQEQFRSDNRRQAISLELMNELKKCPKVLYHVITEEWKQKLKSVPE |
| | ELETKNLHYNTKKEAIIEFEDYDLYIESLTKQVRYNNRFSEFALKYIDETGIFSEFRFQLDLGKILVDEYLKNF |
| | NDERVQRCIIENAKAFGKLNDYTNETKVMSLIVNGPPLKSFDRFAPHYNIESNKIGISTHEVTAKLVPNSKGE |
| | PAKKLHQPLPEAFLSLHELPKIILLEYLQKGEPEKLINEFILVNNSKLMNMSFIEEVKNQLPKEWDKFQRRTN |
| | AKHELAYDENTLAFLLQRKQILNKTLLAYQLNDKQIPTRILDYWLNISDADEERAISNRLKSIK |
| IMG_ | MEILTIPEVIKCRTLSDDPQYFGGYLNMARLNVLNISNHIAKEFKLPLLPEEAHLKNSFLCKKENKKIDWNH |
| 3300029983 | VYARTIRFLSVMKVFDAESLPKEEQKTIDWEGKDFASMCDTLNIVFSELQEFSNDYSHYYSTEKETKRKTT |
| SEQ ID NO: | VSDELALFLRTNFKRAIEYTKVRFKGILNDEDYQLVVSKKMLETNHTITHEGLVFLTSMFLEREYAFRFIRKI |
| 4380 | HGLSGPKDNSFIATCEVLMAFCLKLPQEQFRSDNRRQAISLELMNELKKCPKVLYHVITEEWKQKLKSVPE |
| | ELETKNLHYNTKKEAIIEFEDYDLYIESLTKQVRYNNRFSEFALKYIDETGIFSEFRFQIDLGK |
| IMG_ | MEILTIPEVIKCRTLSDDPQYFGGYLNMARLNVLNISNHIAKEFKLPLLPEEAHLKNSFLCKKENKKIDWNH |
| 3300028767_3 | VYARTIRFLSVMKVFDAESLPKEEQKTIDWEGKDFASMCDTLNIVFSELQEFSNDYSHYYSTEKETKRKTT |
| SEQ ID NO: | VSDELALFLRTNFKRAIEYTKVRFKGILNDEDYQLVVSKKMLETNHTITHEGLVFLTSMFLEREYAFRFIRKI |
| 4381 | HGLSGPKDNSFIATCEVLMAFCLKLPQEQFRSDNRRQAISLELMNELKKCPKVLYHVITEEWKQKLKSVPE |
| | ELETKNLHYNTKRETKIEFEDYDLYIESLTKQVRYNNRFSEFALKYIDETGIFSEFRFQLDLGKILVDEYLKN |
| | FNDERVQRCIIENAKAFGKLNDYTNETKVMSLIVNGPPLKSFDRFAPHYNIESNKIGISTHEVTAKLVPNSKG |
| | EPAKKLHQPLPEAFLSLHELPKIILLEYLQKGEPEKLINEFILVNNSKLMNMSFIEEVKNQLPKEWDKFQRRT |
| | NAKHELAYDENTLAFLLQRKQILNKTLLAYQLNDKQIPTRILDYWLNISDADEERAISNRL |
| IMG_ | MEILTIPEVIKCRTLSDDPQYFGGYLNMARLNVLNISNHIAKEFKLPLLPEEAHLKNSFLCKKENKKIDWNH |
| 3300029989_5 | VYARTIRFLSVMKVFDAESLPKEEQKTIDWEGKDFASMCDTLNIVFSELQEFSNDYSHYYSTEKETKRKTT |
| SEQ ID NO: | VSDELALFLRTNFKRAIEYTKVRFKGILNDEDYQLVVSKKMLETNHTITHEGLVFLTSMFLEREYAFRFIRKI |
| 4382 | HGLSGPKDNSFIATCEVLMAFCLKLPQEQFRSDNRRQAISLELMNELKKCPKVLYHVITEEWKQKLKSVPE |
| | ELETKNLHYNTKRETKIEFEDYDLYIESLTKQVRYNNRFSEFALKYIDETGIFSEFRFQLDLGKILVDEYLKN |
| | FNDERVQRCIIENAKAFGKLNDYTNETKVMSLIVNGPPLKSFDRFAPHYNIESNKIGISTHEVTAKLVPNSKG |
| | EPAKKLHQPLPEAFLSLHELPKIILLEYLQKGEPEKLINEFILVNNSKLMNMSFIEEVKNQLPKEWDKFQRRT |
| | NAKHELAYDENTLAFLLQRKQILNKTLLAYQLNDKQIPTRILDYWLNISDADEERAISNRLKSIKRDCMSRL |
| | KALSKFNENGNRNKIPGIGEMATFLAKDII |
| IMG_ | MEILTIPEVIKCRTLSDDPQYFGGYLNMARLNVLNISNHIAKEFKLPLLPEEAHLKNSFLCKKENKKIDWNH |
| 3300031918_5 | VYARTIRFLSVMKVFDAESLPKEEQKTIDWEGKDFASMCDTLNIVFSELQEFSNDYSHYYSTEKETKRKTT |
| SEQ ID NO: | VSDELALFLRTNFKRAIEYTKVRFKGILNDEDYQLVVSKKMLETNHTITHEGLVFLTSMFLEREYAFRFIRKI |
| 4383 | HGLSGPKDNSFIATCEVLMAFCLKLPQEQFRSDNRRQAISLELMNELKKCPKVLYHVITEEWKQKLKSVPE |
| | ELETKNLHYNTKRETKIEFEDYDLYIESLTKQVRYNNRFSEFALKYIDETGIFSEFRFQLDLGKILVDEYLKN |
| | FNDERVQRCIIENAKAFGKLNDYTNETKVMSLIVNGPPLKSFDRFAPHYNIESNKIGISTHEVTAKLVPNSKG |
| | EPAKKLHQPLPEAFLSLHELPKIILLEYLQKGEPEKLINEFILVNNSKLMNMSFIEEVKNQLPKEWDKFQRRT |
| | NAKHELAYDENTLAFLLQRKQILNKTLLAYQLNDKQIPTRILDYWLNISDADEERAISNRLKSIKRDCMSRL |
| | KALSKFNENGNRNKIPGIGEMATFLAKDIIEMVVSEGKKRKITSFYYDKMQECLALFGDPDKKQLFIHIVTK |
| | ELKLNDPGGHPFLDKLDLQKINSTTGFYEIYLQEKGHKMVPENNPKTGKVIYTDHSWMALTFYKIEFNDKV |
| | DMLMTVVKLPLKKLNI |
| IMG_ | MEILTIPEVIKCRTLSDDPQYFGGYLNMARLNVLNISNHIAKEFKLPLLPEEAHLKNSFLCKKENKKIDWNH |
| 3300030000_4 | VYARTIRFLSVMKVFDAESLPKEEQKTIDWEGKDFASMCDTLNIVFSELQEFSNDYSHYYSTEKETKRKTT |
| SEQ ID NO: | VSDELALFLRTNFKRAIEYTKVRFKGILNDEDYQLVVSKKMLETNHTITHEGLVFLTSMFLEREYAFRFIRKI |
| 4384 | HGLSGPKDNSFIATCEVLMAFCLKLPQEQFRSDNRRQAISLELMNELKKCPKVLYHVITEEWKQKLKSVPE |
| | ELETKNLHYNTKRETKIEFEDYDLYIESLTKQVRYNNRFSEFALKYIDETGIFSEFRFQIDLGKILVDEYLKNF |
| | NDERVQRCIIENAKAFGKLNDYTNETKVMSLIVNGPPLKSFDRFAPHYNIESNKIGISTHEVTAKLVPNSKGE |
| | PAKKLHQPLPEAFLSLHELPKIILLEYLQKGEPEKLINEFILVNNSKLMNMSFIEEVKNQLPKEWDKFQRRTN |
| | AKHELAYDENTLAFLLQRKQILNKTLLAYQLNDKQIPTRILDYWLNISDADEERAISNRLKSIKRDCMSRLK |
| | ALSKFNENGNRNKIPGIGEMATFLAKDIIDMVVSEGKKRKITSFYYDKMQECLALFGDPDKKQLFIHIVTKE |
| | LKLNDPGGHPFLDKLDLQKINSTTGFYEIYLQEKGHKMVPENNPKTGKVIYTDHSWMALTFYKIEFNDKV |
| | DMLMTVVKLPLKKLNI |
| IMG_ | MEILTIPEVIKCRTLSDDPQYFGGYLNMARLNVLNISNHIAKEFKLPLLPEEAHLKNSFLCNKENKKIDWNH |
| 3300030294_3 | VYARTIRFLSVMKVFDAESLPKEEQKTIDWEGKDFASMCDTLNIVFSELQEFSNDYSHYYSTEKETKRKTT |
| SEQ ID NO: | VSDELALFLRTNFKRAIEYTKVRFKGILNDEDYQLVVSKKMLETNHTITHEGLVFLTSMFLEREYAFRFIRKI |
| 4385 | HGLSGPKDNSFIATCEVLMAFCLKLPQEQFRSDNRRQAISLELMNELKKCPKVLYHVITEEWKQKLKSVPE |
| | ELETKNLHYNTKRETKIEFEDYDLYIESLTKQVRYNNRFSEFALKYIDETGIFSEFRFQLDLGKILVDEYLKN |
| | FNDERVQRCIIENAKAFGKLNDYTNETKVMSLIVNGPPLKSFDRFAPHYNIESNKIGISTHEVTAKLVPNSKG |
| | EPAKKLHQPLPEAFLSLHELPKIILLEYLQKGEPEKLINEFILVNNSKLMNMSFIEEVKNQLPKEWDKFQRRT |
| | NAKHELAYDENTLAFLLQRKQILNKTLLAYQLNDKQIPTRILDYWLNISDADEERAISNRLKSIKRDCMSRL |
| | KALSKFNENGNRNKIPGIGEMATFLAKDIIEMVVSEGKKRKITSFYYDKMQECLALFGDPDKKQLFIHIVTK |
| | ELKLNDPGGHPF |
| IMG_ | METLTIPEVIKCRTLSDDPQFFGGYLNMARLNVFNISNHIAKEFNLSLLSEEAHLKDSFLCKKENKKINWNH |
| 3300025153 | VYSQTKRFLSVLKVFDAACLPKEEQKTINWEGKDFASMCDTLNIVFGELQEFSNDYSHYYSTEKGTIRKTT |
| SEQ ID NO: | VSEEMALFLKINFNRAIEYTKEKFKGVLNDEDYLLVASIELFGAENRITTEGLVFLISMFLEREYAFRLIGKIK |
| 4386 | ELLGTQNNCFIAIREVLMAFCLKLPHNRFQSDNTRQAFSLDLINELNRCPKVLYNAIAEEGKKKLRSIPGEPE |
| | NKNLHDNNTKKEAKIEAEAYELYIESLTRQIRYSNRFPDFALKFIDETDIFSEFRFQIDLGKLLVDEYLKFFNG |
| | EQVQRRIIENVKAFGKINDFNDEAKVMNRIGNGHSLKRFEQFAPHYNTENNKIGISRHQSTAKLGSGSKGET |
| | EQKLHQPLPEAFLSLHELPKVILLDYLQKGEPEKLINDFILINNSKLMNMSFIEAVKTQLPPEWDEFQRRTDA |
| | KKEMAYNEKTLAYLLQRKQILNQVLTAYQLNDKQIPGRILDYWLNVTDAEEERAISNRIKSIKRDCMSRLK |
| | ALGKFAENGNRNKIPGIGEMATFLAKDIIDMVVSEGKKRKITSFYYNKMQECLALFADPEKKQLFIHIVTNE |
| | LKLNDSGGHPFLDKLDLQKINSTSNFYEIY |
| IMG_ | METQISNIENKYRILNDDPQYFGGYLNMARLNVFNISNHIAKEFNLPLLPEEGHLKNSFLCQKENKKVNWN |
| 3300028767_6 | HIFSKTNRFLSILKVFDVESLPKEEQKMTDSEGKEFALMSDSLKIVFGELQEFRNDYSHYYSTENGTSRKTT |
| SEQ ID NO: | VSDEMSLFLRTNFLRAIQYTKERFKGVLNDEDYQLVASKKVLEADNTITVEGLVFLTSMFLEREYAFQFIGK |
| 4387 | ITGLKGTQNNSFISTREVLMAFCLKLPHDRFQSDDTRQAFSLDLINELTRCPKELYNAITEEGKMKFQPKLD |
| | EPGIKNLLDNSTNNKKKIDAEDYDEYIESLTKRIRYNNRFSDFALKYIEETDILGDFRFQIDLGKLFVDEYDK |
| | FFNGEEVPRRIIENVKAFGKLNDFNDESILLAQIENGYPSKGFEQFAPHYNTENNKIGISVKVDTAKLRSNSK |
| | GEPGKNLNQPLPEAFLSLNELPKIILLDYLQKGEPEQLINDFILINNSKLMKMSFIEEIKNLLPKEWNEFRKRA |
| | DTRKQAAYNNETLAYLLERKQILNQVLVSYQLNDKQIPGRILDYWLNIKEVEEGRAVSDRLKLMKRDCMS |
| | RLKALEKFKIDRN |
| IMG_ | METQISNIENKYRILNDDPQYFGGYLNMARLNVFNISNHIAKEFNLPLLPEEGHLKNSFLCQKENKKVNWN |
| 3300029998 | HIFSKTNRFLSILKVFDVESLPKEEQKMIDSEGKDFALMSDSLKIVFGELQEFRNDYSHYYSTENGTSRKTT |
| SEQ ID NO: | VSDEMSLFLRTNFLRAIQYTKERFKGVLNDEDYQLVASKKVLEADNTITVEGLVFLTSMFLEREYAFQFIGK |
| 4388 | ITGLKGTQNNSFISTREVLMAFCLKLPHDRFQSDDTRQAFSLDLINELTRCPKELYNAITEEGKMKFQPKLD |
| | EPGIKNLLDNSTNNKKKIDAEDYDEYIESLTKRIRYNNRFSDFALKYIEETDILGDFRFQIDLGKLFVDEYDK |
| | FFNGEEVPRRIIENVKAFGKLNDFNDESILLAQIENGYPSKGFEQFAPHYNTENNKIGISVKVDTAKLRSNSK |
| | GEPGKNLNQPLPEAFLSLNELPKIILLDYLQKGEPEQLINDFILINNSKLMKMSFIEEIKNLLPKEWNEFRKRA |
| | DTRKQAAYNNETLAYLLERKQILNQVLVSYQLNDKQIPGRILDYWLNIKEVEEGRAVSDRLKLMKRDCMS |
| | RLKALEKFKIDRNRSKIPKTGEMATFLAKDIVDMVVSEGIKKKITSFYYDKMQECLALFADPEKKRLFIHIVI |
| | RELRLNGTGGHPFLFQLNFDKINCTSDFYSEYLREKGHKMVKEKNLKTGKIVLTDHSWMALTFYKLEFND |
| | KVDKLMTVVKLPLNKLN |
| IMG_ | METNQQTHENRRRTLTNDPQYFGGYLNMARLNIYNINNHIATDFKQATLPEEGQIPAAFLCNKTIKNLNWN |
| 3300030055_3 | HVHTRAVRFLPILKVFDSESLPKDERENSDTEGKDFASMSDTLKVVFSELQEFRNDYSHYYSTEKQDSRKL |
| SEQ ID NO: | TVSPELANFLTVNFQRAIAYTKARMKDVLTDADYALVENLQMVAPDNKITTEGLVFLIAMFLEREQAFQFT |
| 4389 | GKIQGLKGTQFNSFIATREVLMSFCVKLPHDKFVSENLEQALTLDIINELNRCPKTLYSVITDKEKQQFRPEL |
| | DAQGIDNLIANSTNDDERERILDEIDYQDYIEGLTKRVRYSNRFSYFAMRYIDEKNVFDKLRFHIDLGKYEV |
| | DNYTKQFAGEQAERKVLENA |
| IMG_ | METNQQTHENRRRTLTNDPQYFGGYLNMARLNIYNINNHIATDFKQATLPEEGQIPAAFLCNKTIKNLNWN |
| 3300028651_2 | HVHTRAVRFLPILKVFDSESLPKDERENSDTEGKDFASMSDTLKVVFSELQEFRNDYSHYYSTEKQDSRKL |
| SEQ ID NO: | TVSPELANFLTVNFQRAIAYTKARMKDVLTDADYALVENLQMVAPDNKITTEGLVFLIAMFLEREQAFQFT |
| 4390 | GKIQGLKGTQFNSFIATREVLMSFCVKLPHDKFVSENLEQALTLDIINELNRCPKTLYSVITDKEKQQFRPEL |
| | DAQGIDNLIANSTNDDERERILDEIDYQDYIEGLTKRVRYSNRFSYFAMRYIDEKNVFDKLRFHIDLGKYEV |
| | DNYTKQFAGEQAERKVLENAKAFGKLSSFTDPELIQQRIDKQQHTAGFDQFAPHYNADNNKIGLSTKENIA |
| | TLIAKSKASSKVEHNLKQPLPQAFLSLHELPKIILLEYLQKGQAEELINDFILLNDTRLMDITFIEEVKSQLPA |
| | DWDEFSKRSDAKKKKAYSDSTLKYLRQRKTTLNTILSKSNLNDKQIPTRILNYWLNIKEVDDKRSVSDRIK |
| | LMKRDCMTRLKAVEKHKLNKSVKTPKVGEMATFLAKDIVDMIVSEEKKQKITSFYYDKMQECLALFANA |
| | EKKALFIH |
| IMG_ | METNQQTHENRRRTLTNDPQYFGGYLNMARLNIYNINNHIATDFKQATLPEEGQIPAAFLCNKTIKNLNWN |
| 3300028764 | HVHTRAVRFLPILKVFDSESLPKDERENSDTEGKDFTSMSDTLKVVFSELQDFRNDYSHYYSTEKGDSRKLI |
| SEQ ID NO: | VSPELANFLTVNFQRAIAYTKARMKDVLTDTDYALVENLQMVAPDNKITTEGLVFLIAMFLEREQAFQFTG |
| 4391 | KIQGLKGTQFNSFIATREVLMSFCVKLPHDKFVSENLEQALTLDIINELNRCPKTLYSVITDKEKQQFRPELD |
| | AQGIDNLIANSTNDDERETILDEIDYQDYIEGLTKRVRYSDRFSYFAMRYIDEKNVFDKLRFHIDLGKYEVD |
| | NYTKQFAGEQAERKVLENANAFGKLSSFTDPELIQQRIDKQQHTAGFDQFAPHYNADNNKIGLSTKENIAT |
| | LIAKSKASSKVEHNLKQPLPQAFLSLHELPKIILLEYLQKGQAEELINDFILLNDTRLMDITFIEEVKSQLPAN |
| | WDEFSKRSDAKKKKAYSDSTLKYLRQRKTTLNTILSKSNLNDKQIPTKILNYWLNIKEVDDKRSVSDRIKL |
| | MKRDCMT |
| IMG_ | METNQQTHENRRRTLTNDPQYFGGYLNMARLNIYNINNHVATDFKRKPLPEEGHIPDALLCNKTIKNLNW |
| 3300030339_5 | NHVHTRALRFLPILKVFDSESLPKDERENSDTEGKDFTSMSDTLKVVFSELQDFRNDYSHYYSTEKGDSRK |
| SEQ ID NO: | LIVSPELANFLTVNFQRAIAYTKARMKDVLTDTDYALVENLQMVAPDNKITTEGLVFLIAMFLEREQAFQF |
| 4392 | TGKIQGLKGTQFNSFIATREVLMSFCVKLPHDKFVSENLEQALTLDIINELNRCPKTLYSVITDKEKQQFRPE |
| | LDAQGIDNLIANSTNDDERETILDEIDYQDYIEGLTKRVRYSDRFSYFAMRYIDEKNVFDKLRFHIDLGKYE |
| | VDNYTKQFAGEQAERKVLENANAFGKLSSFTDPELIQQRIDKQQHTAGFDQFAPHYNADNNKIGLSTKENI |
| | ATLIAKSKASSKVEHNLKQPLPQAFLSLHELPKIILLEYLQKGQAEELINDFILLNDTRLMDITFIEEVKSQLP |
| | ANWDEFSKRSDAKKKKAYSDSTLKYLRQRKTTLNTILSKSNLNDKQIPTRILNYWLNIKEVDDKRSVSDRI |
| | KLMKRDCMTRLKAVEKHKLNKSVKIPKVGEMATFLAKDIVDMIVSEEKKQKITSFYYDKMQECLALFAN |
| | AEKKALFIHIVTNELKLFENGGHPFLQNIN |
| IMG_ | METNQQTHENRRRTLTNDPQYFGGYLNMARLNIYNINNHVATDFKQATLPEEGQIPASFLCNKTIKNLNW |
| 3300025888 | NHVHTRALRFLPILKVFDSESLPKDERENSDSEGKDFASMSDTLKVVFSELQDFRNDYSHYYSTEKQDSRK |
| SEQ ID NO: | LTVSPELANFLTVNFKRAIAYTKARMKDVLTDADYALVENLQMVAADNRIATEGLVFLIAIFLEREQAFQFI |
| 4393 | GKIQGLKGTQFNSFIATREVLMSFCVKLPHDKFVSENLEQALTLDIINELNRCPKTLYSVITDKEKQQFRPEL |
| | DAQGIDNLIANSTNDDERETILDEIDYQDYIEGLTKRVRYSNRFSYFAMRYIDEKNVFDKLRFHIDLGKYEV |
| | DNYTKQFAGEQAERKVLENANAFGKLSSFTDPELIQQRIDKQQHTAGFNQFAPHYNADNNKIGLSTKENIA |
| | TLIAKSKASSKVEHNLKQPLPQAFLSLHELPKIILLEYLQKGQAEELINDFILLNDTRLMDITFIEEVKSQLPA |
| | NWDEFSKRSDAKKKKAYSDSTLKYLRQRKTTLNTILSKSNLNDKQIPTRILNYWLNIKEVDDKRSVSDRIK |
| | LMKRDCMTRLKAVEKHKLNKSVKIPKVGEMATFLAKDIVDMIVSEEKKKKITSFYYDKMQECLALFANAE |
| | KKALFIHIVTNELKLFENGGHPFLQNINLQQIRKTSQFYQAYLVEKGNKMVPRLNPKTNKTSKVDESWMM |
| | KQFYVKEWKEEIGKQLTVVKLPANKSQIPFTIRQWDEKEKYDLNVWLQHVTVGKNKDGKKAVNLPTNLF |
| | DEALCDLLREQLDTKIVNYNPAANYNELLKIWWKTRNDDTQQFYQSEREYDIYN |
| IMG_ | METNQQTHENRRRTLTNDPQYFGGYLNMARLNIYNINNHVATDFKRKPLPEEGQIPDALLCNKTIKNLNW |
| 3300028651 | NHVHTRALRFLPILKVFDSESLPKDERKNSDTEGKDFTSMSDTLKVVFSELQEFRNDYSHYYSTEKGDSRK |
| SEQ ID NO: | LIVSPELANFLTINFKRAITYTKARMKDVLTDADYALVENLQMVATDNKITTEGLVFLIAMFLEREQAFQFI |
| 4394 | GKIQGLKGTQFNSFIATREVLMSFCVKLPHDKFVSENQEQALTLDIINELNRCPKTLYSIITDKEKQQFRPEL |
| | DAQGIENLIANSTNNEERERILDEIDYQDYIEGLTKRVRYNNRFSYFAMRYIDEKNIFDKLRFHIDLGKYEVD |
| | NYTKQFAGEQAERKVLENAKAFGKLSSFTDPELSQQRIDKQQQTAGFEQFAPRYHADNNKIGLSNKENIAT |
| | LTAKSKAGSKVEHNLKQPLPQAFLSLHELPKIILLEYLQKGQAEELINDFILLNDTRLMDITFIEEVKSQLPDG |
| | WNEFNKRSDAKKKKAYSDSTLRYLHQRKTILNTILSKYNLNDKQIPTRILNYWLNIKEVDD |
| IMG_ | METNQQTHENRRRTLTNDPQYFGGYLNMARLNIYNINNHVATDFKRKPLPEEGQIPDALLCNKTIKNLNW |
| 3300028868 | NHVHTRALRFLPILKVFDSESLPKDERKNSDTEGKDFTSMSDTLKVVFSELQEFRNDYSHYYSTEKGDSRK |
| SEQ ID NO: | LIVSPELANFLTINFKRAITYTKARMKDVLTDADYALVENLQMVATDNKITTEGLVFLIAMFLEREQAFQFI |
| 4395 | GKIQGLKGTQFNSFIATREVLMSFCVKLPHDKFVSENQEQALTLDIINELNRCPKTLYSIITDKEKQQFRPEL |
| | DAQGIENLIANSTNNEERERILDEIDYQDYIEGLTKRVRYNNRFSYFAMRYIDEKNIFDKLRFHIDLGKYEVD |
| | NYTKQFAGEQAERKVLENAKAFGKLSSFTDPELSQQRIDKQQQTAGFEQFAPRYHADNNKIGLSNKENIAT |
| | LTAKSKAGSKVEHNLKQPLPQAFLSLHELPKIILLEYLQKGQAEELINDFILLNDTRLMDITFIEEVKSQLPDG |
| | WNEFNKRSDAKKKKAYSDSTLRYLHQRKTILNT |
| IMG_ | METNQQTHENRRRTLTNDPQYFGGYLNMARLNIYNINNHVATDFKRKPLPEEGQIPDALLCNKTIKNLNW |
| 3300028664 | NHVHTRALRFLPILKVFDSESLPKDERKNSDTEGKDFTSMSDTLKVVFSELQEFRNDYSHYYSTEKGDSRK |
| SEQ ID NO: | LIVSPELANFLTINFKRAITYTKARMKDVLTDADYALVENLQMVATDNKITTEGLVFLIAMFLEREQAFQFI |
| 4396 | GKIQGLKGTQFNSFIATREVLMSFCVKLPHDKFVSENQEQALTLDIINELNRCPKTLYSIITDKEKQQFRPEL |
| | DAQGIENLIANSTNNEERERILDEIDYQDYIEGLTKRVRYNNRFSYFAMRYIDEKNIFDKLRFHIDLGKYEVD |
| | NYTKQFAGEQAERKVLENAKAFGKLSSFTDPELSQQRIDKQQQTAGFEQFAPRYHADNNKIGLSNKENIAT |
| | LTAKSKAGSKVEHNLKQPLPQAFLSLHELPKIILLEYLQKGQAEELINDFILLNDTRLMDITFIEEVKSQLPDG |
| | WNEFNKRSDAKKKKAYSDSTLRYLHQRKTILNTILSKYNLNDKQIPTRILNYWLNIKEVDDKRSVSDRIKL |
| | MKRDCMTRLKAVEKHKLNKSVKIPKVGEMATFLAKDIVDMIVSEEKKKKITSFYYDKMQECLALFTNAEK |
| | KALFIHIVTNELKLLENDGHPFLQNINLQQIRKTSQFYQAYLIEKGNKMVPRLNPKTNKTSKVDESWMMKQ |
| | FYVKEWKEEIGKQLTVVKLPANKSRIPFTICQWDKKESHDLNTWLQHVTVGKNKDGKKAVNLPTNLFDE |
| | ALCDLLREQLD |
| IMG_ | METNQQTHENRRRTLTNDPQYFGGYLNMARLNIYNINNHVATDFKRKPLPEEGQIPDALLCNKTIKNLNW |
| 3300028665 | NHVHTRALRFLPILKVFDSESLPKDERKNSDTEGKDFTSMSDTLKVVFSELQEFRNDYSHYYSTEKGDSRK |
| SEQ ID NO: | LIVSPELANFLTINFKRAITYTKARMKDVLTDADYALVENLQMVATDNKITTEGLVFLIAMFLEREQAFQFI |
| 4397 | GKIQGLKGTQFNSFIATREVLMSFCVKLPHDKFVSENQEQALTLDIINELNRCPKTLYSIITDKEKQQFRPEL |
| | DAQGIENLIANSTNNEERERILDEIDYQDYIEGLTKRVRYNNRFSYFAMRYIDEKNIFDKLRFHIDLGKYEVD |
| | NYTKQFAGEQAERKVLENAKAFGKLSSFTDPELSQQRIDKQQQTAGFEQFAPRYHADNNKIGLSNKENIAT |
| | LTAKSKAGSKVEHNLKQPL |
| IMG_ | METNQQTHENRRRTLTNDPQYFGGYLNMARLNIYNINNHVATDFKRKPLPEEGQIPDALLCNKTIKNLNW |
| 3300030294_4 | NHVHTRALRFLPILKVFDSESLPKDERKNSDTEGKDFTSMSDTLKVVFSELQEFRNDYSHYYSTEKGDSRK |
| SEQ ID NO: | LIVSPELANFLTINFKRAITYTKARMKDVLTDADYALVENLQMVATDNKITTEGLVFLIAMFLEREQAFQFI |
| 4398 | GKIQGLKGTQFNSFIATREVLMSFCVKLPHDKFVSENQEQALTLDIINELNRCPKTLYSIITDKEKQQFRPEL |
| | DAQGIENLIANSTNNEERERILDEIDYQDYIEGLTKRVRYNNRFSYFAMRYIDEKNIFDKLRFHIDLGKYEVD |
| | NYTKQFAGEQAERKVLENAKAFGKLSSFTDPELSQQRIDKQQQTAGFEQFAPRYHADNNKIGLRNKENIAT |
| | LAAKSKAGSKVEHNLKQPLPQAFLSLHELPKIILLEYLQKGQAEELINDFILLNDTRLMDITFIEEVKSQLPD |
| | GWNEFNKRSDAKKKKAYSDSTLRYLHQRKTILNTILSKYNLNDKQIPTRI |
| IMG_ | MEANEQNQENRRRTLTNDPQYFGGYLNMARLNIYNINNHIAADFGQAVLPEEGQIPSGFLCNKEIKKLNW |
| 3300030055_4 | NHIYAKTRRFLPILKVFDIESLPKEEQVNSDKEGKDFAAMSDTLKVVFSELQDFRNDYSHYYSTEKGENRK |
| SEQ ID NO: | LTISAELTDMLTINFKRAIAYTKVRMKDVLTDADYELVETKQVVTTGNIITTEGLVFLTCMFLEREHAFQFI |
| 4399 | GKIQGLKGTQFNSFIATREVLMSFCVKLPHDKFVSENLEQALTLDIINELNRCPKTLYGVITDEEKMQFRPEL |
| | DELDIEKLIANSTNDDERERILDEIGYEEYIEGLTKRVRYNNRFPYFAMRFIEEKNVFDKLRFHIDLGKYEVD |
| | RYTKQLAGEQTERVVQENVKAFGKLSSFTDPELIQQKIDNQQRTDGFE |
| IMG_ | MEANEQNQENRRRTLTNDPQYFGGYLNMARLNIYNINNHIAADFGQAVLPEEGQIPSGFLCNKEIKKLNW |
| 3300030943_3 | NHIYAKTRRFLPILKVFDIESLPKEEQVNSDKEGKDFAAMSDTLKVVFSELQDFRNDYSHYYSTEKGENRK |
| SEQ ID NO: | LTISAELTDMLTINFKRAIAYTKVRMKDVLTDADYELVETKQVVTTGNIITTEGLVFLTCMFLEREHAFQFI |
| 4400 | GKIQGLKGTQFNSFIATREVLMSFCVKLPHDKFVSENLEQALTLDIINELNRCPKTLYGVITDEEKMQFRPEL |
| | DELDIEKLIANSTNDDERERILDEIGYEEYIEGLTKRVRYNNRFPYFAMRFIEEKNVFDKLRFHIDLGKYEVD |
| | RYTKQLAGEQTERVVQENVKAFGKLSSFTDPELIQQKIDNQQRTDGFEQFAPHYNADNNKIGLSNKESIAIL |
| | IPKSKPESKVGNNLKQPLPQAFLSLHELPKIILLDYLQKGKAEELINDFILLNDTRLMDITFIEEVKLKLPANW |
| | NEFAKRSDAKKKKAYSDAAMEYLLQRKATLNDVLITYNLNDKQIPTRILNYWLNIKDVEDNRSV |
| IMG_ | MEANEQNQENRRRTLTNDPQYFGGYLNMARLNIYNINNHIAADFGQAVLPEEGQIPSGFLCNKEIKKLNW |
| 3300028864_3 | NHIYAKTRRFLPILKVFDIESLPKEEQVNSDKEGKDFAAMSDTLKVVFSELQDFRNDYSHYYSTEKGENRK |
| SEQ ID NO: | LTISAELTDMLTINFKRAIAYTKVRMKDVLTDADYELVETKQVVTTGNIITTEGLVFLTCMFLEREHAFQFI |
| 4401 | GKIQGLKGTQFNSFIATREVLMSFCVKLPHDKFVSENLEQALTLDIINELNRCPKTLYGVITDEEKMQFRPEL |
| | DELDIEKLIANSTNDDERERILDEIGYEEYIEGLTKRVRYNNRFPYFAMRFIEEKNVFDKLRFHIDLGKYEVD |
| | RYTKQLAGEQTERVVQENVKAFGKLSSFTDPELIQQKIDNQQRTDGFEQFAPHYNADNNKIGLSNKESIAIL |
| | IPKSKPESKVGNNLKQPLPQAFLSLHELPKIILLDYLQKGKAEELINDFILLNDTRLMDITFIEEVKLKLPANW |
| | NEFAKRSDAKKKKAYSDAAMEYLLQRKATLNDVLITYNLNDKQIPTRILNYWLNIKDVEDNRSV |
| IMG_ | MKSNEQTYENKRRTLTNDPQYFGGYLNMVRLNIYNISNHIASDFGQAQLPEEGQIPTSFLCNKGIKKLNWN |
| 3300029923 | HVYTKTRRFLPILKVFDAESLPKEERENYEKEGKDFAAMSDTLKVVFTELQAFRNDYSHYYSTEKGENRKL |
| SEQ ID NO: | TVSGELADFLTINFKRAIAYTKVRMKDVLTDADYELVENRQIVVDNNTITTEGLVFLISMFLEREQAFQFIG |
| 4402 | KIQGLKGTQFNSFIATREVLMSFCVKLPHDKFVSEDLEQALTLDIINELNRCPKTLYKVSTEEAKLQFRPELD |
| | AQGIDNLLANSTNIDECEKILDEINYEDYIEGLTKRVRHNNRFSYFAMRYIDEKNVFEKLRFHIDLGKYEVD |
| | TYTKQLAGEQTERVVFENVKAFGKLNSFTDSESVQQRIDKQQRTGGFEQFAPHYNAENNKIGLSSKEEVAL |
| | LLPKSKPDTKVAYNLKQPLPQAFLSLHELPKVILLEYLQKGKSEQMINDFILLNDTRLMDMTFIEEVKSKLP |
| | FGWNEFTKRSDAKKKKAYSNATMKYLLQRKTIVNDVLIDYNLNHKQIPTRILDYWLNIKDVEDSRSVSDRI |
| | KLMKRDCMTRVKVLEKHKLDKSVKTPKVGEMASFLAKDIVDMIVSKEKKQKITSFYYDIMQECLALFAD |
| | AEKKALFIHIVTNELKLFENGGHPFIQNINLQQLHKTSQFYEAYLKEKGNKQVSKFNPKTNKTSKVDDSWM |
| | MQQFYTKEWNDEIKKQLTVVKLPANKTHIPFTIRMWEEKEKYNLETWLHNVTVGKNIKDGKKAVNLPTN |
| | LFDEALCILLRKQLDTLAPNYNPAANYNELLKLWWKTRNDDTQDFY |
| IMG_ | MENDQQILENRRRTLANDPQYFGGYLNMARLNIYNISNHLATSFEQKALHEEGQIPASFLCNKSIKKINWN |
| 3300030001 | HVYSKARRFLPILKIFDADSLPKEERETSDKEGKDFTAMNETLKLVFDELQAFRNDYSHYYSTEKADSRKL |
| SEQ ID NO: | TISVELADFLTVNFKRAIAYTKVRMKDVLADDDYAVVESKQIVTPDNQITTEGLVFLTCIFLEREQAFQFIG |
| 4403 | KVQGLKGTQFNSFIATREVLLAYCVKLPHDKFVSEDLRQALTLDIINELNRCPKTLYEVITEEEKQQFRPELD |
| | AQGIDNLIANSTNEEEREKILDEIDYEDYIESLTKRVRHSNRFPYFAMRYIEEKNVFDKLRFHIDLGKYEVEK |
| | YNKQFDGEATERKVVENAKAFGKLSSFTNQETVELKIDSAQRTNGFEQFAPHYNADNNKIGLSNKESEARL |
| | LTKAKPESKVSYNLKQPLPQAFLSLHELPKIILLEYLQKGKAEEMINDFIKVNDSQLMNMQFIDEIKEQLPAD |
| | WNEFGKRSDSKKKKAYTNAARQYLLQRKATLNKVLANYQLNDKQVPTRILNYWLNVKEVDDSRSVSDRI |
| | KLMKRDCMSRLKVMEKHKVDKSARTPKVGEMATFLAKDIVDMIVSTDKKQKITSFYYDKMQECLALYA |
| | DNEKKATFIHIVTNELKLLEKDGHPFLANINLRQIRKTSQLYELYLVEKANKQVKKMNPKTQRTNNVDES |
| | WMMKSFYAKEWNEEMGKQLTVVKLPANKTNIPFTIRQWEEKEKHNLQAWLHNITKGKTSKDGKKAVDL |
| | PTNLFDDTLCELLREALINEGIDATP |
| IMG_ | MENDQQILENRRRTLANDPQYFGGYLNMARLNIYNISNHLAASFEQKVLPEEGQIPASFLCNKSIKKINWN |
| 3300031902 | HVYSKARRFLPILKVFDADSLPKEERETTDKEGKDFTAMNETLKLVFDELQAFRNDYSHYYSTEKADSRKL |
| SEQ ID NO: | TISVELADFLTVNFKRAIAYTKVRMKDVLADDDYTMVESKQIVTPDNLITTEGLVFLTCMFLEREQAFQFIG |
| 4404 | KVQGLKGTQFNSFIATREVLLAYCVKLPHDKFVSEDLGQALTLDIINELNRCPKTLYEVITEEEKQQFRPEL |
| | DAQGIDNLIANSTNEEEREKILDEIDYEDYIESLTRRVRHSNRFSYFAMRYIEEKNVFDKLRFHIDLGKYEVD |
| | KYNKQFDGEATECKVVENAKAFGKLSSFTNQETVELKIDSAQRTNGFEQFAPHYNADNNKIGLSNKESEA |
| | RLLTKAKPESKVSYNLKQPLPQAFLSLHE |
| GCA_ | MDTIEKTEHKGLNVYKTLETDPQYFGGYLNMARLNIFSINNYVADKLKISALVNEEKMLDSFLCNNNRKH |
| 002400765.1_ | LNWNLAHSIAVKFFPIMKVFDFESLPKLERTVDLNNINTGKDFVAMAVVLRYLFREIQEFRNDYSHYYSIVN |
| ASM240076v1_ | GNKRKTIISREVAEFLRLNFTRAIEYTKERFNGVLNNEDFEYVKERVLVNQDNTITTDGFVFLISMFLEREHA |
| genomic_2 | FQFIGKIKGLKGTQYSSFIATREVFMAFCVKLPHDRFVSEDKRQALTLDIINTLNRCPKELYTVITDEERKVF |
| SEQ ID NO: | KPSLDSLKLKNLLDNSTNDQADIEDYDNYIEVLTRKIRHSNRFSFFALKFIDETDIFSKLRFHINLGKLLIEEY |
| 4405 | EKPINNELYPRSIVQNVKAFGKLSDFEDGIEVLKQIDKEGNSLGFEQYAPFYNTKNNKIGLHTNSAKSIVINK |
| | PKSESKIKKSLKQALPEAFLSLHELPKIIVLEYLAKGKSEELINDFILICNSKIINKQFIDEVKGELPKDWNEFN |
| | KRSDSKKDPAYKPNALAYLIKRKKIVDEVLAQYNLNHKQIPTRILDYWLCIVDRNADRAISERIKRMKREG |
| | MDRLKAYRKFKKTGKGKIPKIGEMATFMAKXXXXXXXXXXXXXKXXXXXXXXXXXXXXPCLPIPKKNS |
| | YLLILSAKSFGLTR |
| IMG_ | MDTIERIEIKSANVYKTLENDPQYFGAYLNMARLNIFSINNSVADKIKVAPIPNEEKILDSFLCNHNRKHLN |
| 3300027758 | WNLAHAIAVKFLPIIKVFNFEGLPKSERTSDFNNINTGKDFAAMADALRSLFGEIQEFRNDYSHYYSITNGN |
| SEQ ID NO: | KRKTTISKEVAEFLNKNFARAIEYTKDRFNGVLNNEDFYHVKERVLVNKDNTITTDGLVFLIAMFLEREHA |
| 4406 | FQFIGKIKGLKGTQYNSFIATREVLMAFCAKLPHDRFVSEDKKQAFTLDIINTLNRCPKELYAVITEEERKAF |
| | KPNLDSLKIENLLNNSTNDRADIENYDKYIEALTRKVRHSNRFSYCALKFIDETNIFKQLRFQINLGKLGLDE |
| | YEKPINNELYPRSIVQNVKAFGKLSDFEDEKEVLKQIDKEGNSLGFDQYAPFYNTKNNKIGLHTNNAKSIVI |
| | NKAKSESKIKNKLKKALPEAFLSLNELPKIIVLEYLEKGKSEELINDFILASNSKITNKQFIDEVKGKLPNDW |
| | NEFNKRSDSKKETAYKPNALAYLRNRKKILDEVLAQYNLNHKQIPTRILDYWLSVVDINSERAISDRIKRM |
| | KREGMDRLKSYQKYKKTRKGRIPKIGEMATFLAKDIIDMIISTDKKKKITSFYYDKMQECLALFADPDKKA |
| | LFIDIISKELHLNELDGHPFLKYIRFSKISYTQDLYESYLQEKANKMIDVKNHRTGRTNQIDKSWMMTTFYR |
| | REWNKEAGKQLTEVKLPHNLSCIPFSLRQLKEKTSNNLDEWLHNITKGKEVNDGKKPINLPTNLFDETLIRL |
| | LKSDLDTQHEQYPEDAKYNELFKIWWRKRGDSTQSFYNAEREYLIYDEKVNFKLQENAEFADFYSDNLRK |
| | AYKAKQADRRI |
| IMG_ | MEENLNSLLSRTRTISNDPQYFGGYLNMARLNIFNISNYIGKLFSQSQLDDDDHIANSFLTNETIKNLNWNH |
| 3300028603 | VFSKALRFLPIVKLFDLEEYPREIIDELGKKFIAPNETKDFHNMRKSLKLIFSNINNFRNDYSHFYSTISGTNR |
| SEQ ID NO: | KLEIEDDIANLLRNAFTFAISHTKLRLKEVLKEEDFNLVSEKKMVEEGNKITTEGLVFLICMFLEREHAFHFI |
| 4407 | KRIIGFRGNHIKSFVATHEVFMTFCVKLPHDKLISEDYEQRLAMDMVKELNNCPKDLYRLLTERERAKLRY |
| | CSPLIGGNHNDVDQLDYDSYREMLVSNIRHRNRFFYFALRFIDETNCFPTLRFHIDIGKLELVSYLKSFAGSE |
| | EERRIVVDVKTFGKLSEFVEEDTLHKKIDKNGYTTGFDQFSPRYNFKLNKIGIRKSGTKFPIILPTIVSKSDQT |
| | GNIKIRLKQYAPDAFLSLHMLPQIILIEYLERGASEKVINDFINKNKEILDKKFIQQIKGELPTDWAKFQKRSD |
| | SKKRPAYDTYNLKSLTDRKQYLNDVLEKHNLNVKQIPTRVLEYWLNLNDVDGSQLFSNRIKLMKKECTDR |
| | LKVIEKSKINPNIRTPKVGEMATFLAKDIVDMIVDSEIKSQCSSFYYHKLQKSLAFYATSDEKKIFSEIKDELK |
| | LIGTGGHPFLSRVLDKSPINTLEFYIYYLKEKADTRTYKTEKNNSWIEKTFYTTVKDKKTKKRMVTVRMPE |
| | NASNIPYTIQ |
| IMG_ | METTTVAEFSKTRTMESDPQYFGSYLNMARHNIFNISNYIADYFNLSRLKDDDLIQNSFLCNPDIQKINWPY |
| 3300019861 | VFGRTKHLLSILKVFDTDTLPKDEVLSSSQAGKQFLLMNETLKLVFRELQQFRNDYSHYYSAEKGSDKKIII |
| SEQ ID NO: | DEQLVQFLNLNFKRAISYTRERFKDVFSEDDFKYAINLKLVKEDNKITVHGMVFLIAMFLEREEAFSFISRIS |
| 4408 | GLKGTYSKSFLATREVLMAFCVKLPHDKFKSNDEKQATSLDLINELNRCPLDLWNNLNQSDKMKFIPDLE |
| | VDENGDHLAEEYEIYAETITKQIRYKNRFTEFALKYIDYAGVLPKYKMLIDVGKISLGSYTKIMNNEPYEREI |
| | QDEVIAFDKTVEYTKKDEVLKRVDAEKRTKGFTRFNPHYSSAANKIGLLYKSDFSQVMPAQDRKLGIRLN |
| | HPAARAFLSANELTKVILLDYLIPREPERIINRFIQKNKQILDLNFINQIKEQIGFNEFARRTSKKNEHAYTEGA |
| | LNHLTFRKNQLQAILSKYNLTIAQIPSKIIDYWLNIKPVDEYRKAAERITRIRLETKTRYKEHLKARIANKPAL |
| | KQGIMASYLARDII |
| IMG_ | MEDLILEHRDKGKSKNNETQSKRTLGNDPQYFGAYLNMARHNIFTINNHLVKKLKLQDTLVLSDEESIPDS |
| 3300003541 | FLVKKIKEKPNLLFTQLIRFLPIAKVFNPELLPKEEQEKEKEENIDFKSLADTLKICFGELNKFRNDYTHYYSK |
| SEQ ID NO: | TNGLDRKIIIDENLAVFLRINKTRAIEYTKKRFKDIFEDKHFIITEKKELVDQSSKITQDGLVFFICLFLDRENA |
| 4409 | FQFINRIIGFKDTRTPEYKATREVFSSFCVNLPHDKFISDDPVQAFILDMLNELNRCPLELYNNITKKEKKQFQ |
| | PDISDKISNIEENSIPEEISVDKYEEYIQNITTKIRRKDRFPYFALKYLDMKDDYQLKFHINLGKALLDTHKKL |
| | CLGKEENREIVEDVKIFGKLKDFENEDKIIKNIDKKKKMEFKQFNPHFHIENNKIGFSFNLKSCSIKYGLSEKP |
| | NLKLSIPDGFLSINELPKVLLLELLKKGKSIEIIKSFLNTNRENILNKEFIERVKEDLVFEKSFYRSFQKKKEPA |
| | YSEKALSILKDRKTKLNSLLRQHNLNDKQIPARILNYWLDIKPVKEEMSIANKIKAMKKDCIDRLKAKKKN |
| | KAPKVGEMATYLAHDIVDMIIDEKLKNKITSFYYDKMQECLALFSDEEKKQLFLQICEKELNLFDEKKGHP |
| | FLKELDLYNINKTSDIYEKYLEKKGNNMKTLKNEKQQKSYQSDTSWLYTTFYVKSKNPTTNKWETKVNLP |
| | PDLSKLPFSIRNLLRKKSNFEQWLKNVTDGYSDNDKP |
| IMG_ | MENLNKRTLTTDPQYFGGYLNSARHNIFTISNYIAERINPLMKKGKLSIRKDDDEIADSFICTKIIEKPNLFFT |
| 3300032420 | NLVRFLPIVKVYDSDKLPKAEKEKPSSEGIDFETLADDMKICFKELNGFRNDYSHYFSKETGTERKIVIDERL |
| SEQ ID NO: | SVFLRTNYQRAIEYTKIRFKDVYEESHFKIAADKILVNESNVITQDGLVFFTCLFLDRENAFHFINRIIGFKDT |
| 4410 | RTLGFRATREVFSAYCVTLPHDKFTSDDEKQGFILDLLNELNKCPKELYDNITEEERKIFRPDVSESIDKITES |
| | SIPEDLAFEDYDEYIQSIITKKRKSDRFPYFAIKYLDGKKDFDINFHLNLGKVELLSRKKKFLGEEVDRDIVE |
| | DVKVFGKLAEYTNEKEVSRKLGLEFQLFNPHYQIENNKMGISFSPKLCSVKSENDKPNLKLNPPDAFLSVH |
| | ELPKIVLAELFEKGKAKEIIESFIGINKDKILNREFIEEVKSKLVFEKPFYRSFQSKRGAAYNDKGLQILKERK |
| | TKLNEILREYNLNDRQIPERILDYWLNINDVKSESEIANRIKAMKKDCRDRVKAKAKNKAPKAGEMATYL |
| | AKDIVDMVIDEKVKQKITSF |
| GCA_ | MERIFGHCCPHHDSVCFVRFLGTMVSNQDGRENVLDILYIADRTRKLKPMNTVPASENKGQSRTVEDDPQ |
| 002529355.1_ | YFGLYLNLARENLIEVESHVRIKFGKKKLNEESLKQSLLCDHLLSVDRWTKVYGHSRRYLPFLHYFDPDSQI |
| ASM252935v1_ | EKDHDSKTGVDPDSAQRLIRELYSLLDFLRNDFSHNRLDGTTFEHLEVSPDISSFITGTYSLACGRAQSRFAD |
| genomic | FFKPDDFVLAKNRKEQLISVADGKECLTVSGLAFFICLFLDREQASGMLSRIRGFKRTDENWARAVHETFC |
| SEQ ID NO: | DLCIRHPHDRLESSNTKEALLLDMLNELNRCPRILYDMLPEEERAQFLPALDENSMNNLSENSLNEESRLLW |
| 4411 | DGSSDWAEALTKRIRHQDRFPYLMLRFIEEMDLLKGIRFRVDLGEIELDSYSKKVGRNGEYDRTITDHALAF |
| | GKLSDFQNEEEVSRMISGEASYPVRFSLFAPRYAIYDKR |
| GCA_ | MNTVPASENKGQSRTVEDDPQYFGLYLNLARENLIEVESHVRIKFGKKKLNEESLKQSLLCDHLLSVDRWT |
| 002529355.1_ | KVYGHSRRYLPFLHYFDPDSQIEKDHDSKTGVDPDSAQRLIRELYSLLDFLRNDFSHNRLDGTTFEHLEVSP |
| ASM252935v1_ | DISSFITGTYSLACGRAQSRFADFFKPDDFVLAKNRKEQLISVADGKECLTVSGLAFFICLFLDREQASGMLS |
| genomic_2 | RIRGFKRTDENWARAVHETFCDLCIRHPHDRLESSNTKEALLLDMLNELNRCPRILYDMLPEEERAQFLPAL |
| SEQ ID NO: | DENSMNNLSENSLNEESRLLWDGSSDWAEALTKRIRHQDRFPYLMLRFIEEMDLLKGIRFRVDLGEIELDS |
| 4412 | YSKKVGRNGEYDRTITDHALAFGKLSDFQNEEEVSRMISGEASYPVRFSLFAPRYAIYDKR |
| IMG_ | MEEQFLQKERNMGDNPYYFCHFINMAHHNVNLILEEIYNSVYEKYTQDKEENIKAICNSMISKSRKNPDEK |
| 3300029998_2 | AKMMNMCIRHFPFLDYYKEKDQNSDVLTILLNQFLVPLHGLRNQFSHYKHPQEAYCISGFDLLFEQAKTG |
| SEQ ID NO: | AQMRMKYSDEDISKVKSKVVNHDSILTERGILFFICLFLDKRNIYLFLSKIKGFRDRRPDEKYKSATLEVFSQ |
| 4413 | YYCHVPYRKLDSSDVALDMLNELNRCPKALYDVLSDEDRERFIVDNVENADNRDEISDEDDEEMPRSVMK |
| | RSDDRFPYFALRYFEKQNNLDEISFHLYLGRKEAKPAHEKVINGEMRTHKILKDIHVFGRLENYRNEEICNA |
| | IKNREDIEFYAPSYRIVENRIGLLLRRQNDFTLEEANEEKIFEGNLCPDVILSTHELGALFFYNYLH |
| IMG_ | MEEQFLQKERNMGDNPYYFCHFINMAHHNVNLILEEIYNSVYEKYTQDKEENIKAICNSMISKSRKNPDEK |
| 3300030673_3 | AKMMNMCIRHFPFLDYYKEKDQNSDVLTILLNQFLVPLHGLRNQFSHYKHPQEAYCISGFDLLFEQAKTG |
| SEQ ID NO: | AQMRMKYSDEDISKVKSKVVNHDSILTERGILFFICLFLDKRNIYLFLSKIKGFRDRRPDEKYKSATLEVFSQ |
| 4414 | YYCHVPYRKLDSSDVALDMLNELNRCPKALYDVLSDEDRERFIVDNVENADNRDEISDEDDEEMPRSVMK |
| | RSDDRFPYFALRYFEKQNNLDEISFHLYLGRKEAKPAHEKVINGEMRTHKILKDIHVFGRLENYRNEEICNA |
| | IKNREDIEFYAPSYRIVENRIGLLLRRQNDFTLEEANEEKIFEGNLCPDVILSTHELGALFFYNYLHKKGWIES |
| | APYLYIRNFISDFKRFIEDIKNGKLTPVESEDDFYLIKKKKRDETKDNDKKSIAVQERRREKLKEKLKGYHLE |
| | PDWIPDACREYMLGYKADQKDYYTKQRFCSMKKETDSRIKQIEAIRKREDNSIIRQTRVGEIAQELARDIVF |
| | LIPPYKNEKGADTKINNMEFDVLQKMLAYFPLNKKDIYPFLKNIRNWDKHPFLKYTLHTEHQSLLDFYQDY |
| | LNCKKRWISKNIRYDKQKGNYLVDANKTEQECRYFLKTDKLRTAKEKEYFEEPDKPVYLPTGFFVDPIVEA |
| | MRKNGYELKENSNIVGCLKIYFVSKIQPMYDLSRYYTYYDGKEERSM |
| IMG_ | MEEQFLQKERNMGDNPYYFCHFINMAHHNVNLILEEIYNSVYEKYTQDKEENIKAICNSMISKSRKNPDEK |
| 3300030685_3 | AKMMNMCIRHFPFLDYYKEKDQNSDVLTILLNQFLVPLHGLRNQFSHYKHPQEAYCISGFDLLFEQAKTG |
| SEQ ID NO: | AQMRMKYSDEDISKVKSKVVNHDSILTERGILFFICLFLDKRNIYLFLSKIKGFRDRRPDEKYKSATLEVFSQ |
| 4415 | YYCHVPYRKLDSSDVALDMLNELNRCPKALYDVLSDEDRERFIVDNVENADNRDEISDEDDEEMPRSVMK |
| | RSDDRFPYFALRYFEKQNNLDEISFHLYLGRKEAKPAHEKVINGEMRTHKILKDIHVFGRLENYRNEEICNA |
| | nCNREDIEFYAPSYRIVENRIGLLLRRQNDFTLEEANEEKIFEGNLCPDVILSTHELGALFFYNYLHKKGWIES |
| | APYLYIRNFISDFKRFIEDIKNGKLTPVESEDDFYLIKKKKRDETKDNDKKSIAVQERRREKLKEKLKGYHLE |
| | PDWIPDACREYMLGYKADQKDYYTKQRFCSMKKETDSRIKQIEAIRKREDNSIIRQTRVGEIAQELARDIVF |
| | LIPPYKNEKGADTKINNMEFDVLQKMLAYFPLNKKDIYPFLKNIRNWDKHPFLKYTLHTEHQSLLDFYQDY |
| | LNCKKRWISKNIRYDKQKGNYLVDANK |
| GCA_ | MDISNEKTSRYKDLENDPYYFNHFINMGRHNAYLILHDVYKTVYKEELSLEENNLAVFRKKVLEKSQNKP |
| 002307035.1_ | DEIAKVINILLRHFPFLAYYEEKQQYVKEKHKYESLNRLADYLGALNKIRNQTSHYKHNKEDIYLPDYQGL |
| ASM230703v1_ | FQMGVKEAQNRMKYEDKDVKHLYRTQYYNLVNNNILTEAGISYFVCLFLDKKNGYLFLSRIKGFKDRNK |
| genomic | TSERYKSATLEAFTQFHSHVPYPKLDSSDIALDMLNELNRCPKQLYNVLSAEDQNKFIATLSEDGDDELPKP |
| SEQ ID NO: | LMKRSEDRFPYFALRYFEKSGKLDNITFQLYLGRKHAQEPHTKKIAGVERIHFLLKNMHVFGKLPFYKEEE |
| 4416 | AHRFYGENEEVEFYAPAFRMVGNRIGLVLREELQSHYTVPATNKTEKDKNYPDAILSTHELSGL |
| IMG_ | LLIRIRFYSFGKNYSNMETPNKTSTSAYKDLQNDPYYFSHFINMGRHNAYLIIHYIYKCVYKEELNLIESNLY |
| 3300025106 | QFSSKVKANSKKNPDELTKVIRLLLLHFPFLAYYDNAEREKRDERVVGRSNDGNWNKKVKERPYLSDKNT |
| SEQ ID NO: | VSSNSEKASEKATSESHICLERLSQFLIVLNNLRNETSHYKHPKKSTLLPDFQKMYQSGIKEAQRRMNYEDK |
| 4417 | DIQHLFKDPHYKLIETQKQTETVGLTKFNGQKKVNRQPQVNGQTGTDKLAKVDKLTGFDKLTEVDELQDL |
| | DELTEIGIYYFICLFLDKKNGYLLLSRIRGFKDRNRTSEKYKSATLEAFTQFHCLVPNPKLESSNIAMDMLNE |
| | LNRCPRQLYQVLSQDDKEKFVATDTEKEEDSDEVPEPIMKRSEDRFPYFALRYFEEMSRLGLSGTLDQITFQ |
| | LFLGRKHDQEPHTKILNGTQRTHSLLKNMHVFGKLPFYQKEEAYQFYEGNEEVEFYAPAYRIVGNRIGLVL |
| | KDIHPHYTIPKSDGNYKNGNCKNGNCKNENCPDAILSTHE |
| IMG_ | MDTPNFSERIPVSLQSHPYYFAHYLNMARHNAYVILEYVNRELIKPGKNLDEDNLIQSTVLKDGYFDRKPD |
| 3300025308_2 | ELSHRNRLLVQHFPFLREAENEGARTCNPVSYKLKTALAALNQWRNNASHYPLNQNHEKDFDLQPFFSFAI |
| SEQ ID NO: | EACKKRMREVFQPDDFYLLETNEKQFYTLHNENGFTEKGLYCFICFFLEKKYAFQFLAGIKGFKNTTDNKF |
| 4418 | RATLETFTEHCCRLPKPKLDSSDIKLDMLGELSRCPAPLFDLLDIEERKKFIREPEEVKPDESGDREEVQQVL |
| | MKRYDDRFPYFALRYFEEKNLLKGISFHIHIGRWIKSEHTKKIMGAERDRRLLKDIRTFGELKEFSPEHEPYR |
| | YKT |
| IMG_ | MNNPENQKEKTSIGTHPFYFGHYLNMARHNAYIILCALSKKYNFNIPDESEQNEAQLNHFKILNFAADKEK |
| 3300027566 | RPDELNAIKEDLQFHFPVLKAFQLSEFGKSFSDLLILLGDLRNRYSHVYYKKDFKHEVELRDILKQARKDAI |
| SEQ ID NO: | KRMNSVIPEEEFHHLVKVKESKIPFKFYLTERDRNTLTEKGVAFLCCLFLEKKFAFRFLSRLENFHRTEEKW |
| 4419 | ARATLETFTEYCCILPYDRLDSSDIKLDMINELNRCPRELYYLLDDSLKKKFLDKPEAEEDLTETSTDENAE |
| | YEKPTPLRRHSDRFPWFALNFFENVYPGIHFQVKLGRVLTQDLYDKTIASTSRDRRILKDINSLGHPFKYPVE |
| | SAPDSWYNIKQASETGLVNTAMRAGEIDQYSPKFRITEKRIGLFLNKPYTIPFWPNLSKEAKPKKSGPIITTC |
| | KASAIKPDAILSTYELQNR |
| IMG_ | METKTSKTSTLMTINKNPYYFGQYCNMAINNIYLILKKVSTKVYGEEKIKTPCNIIEFIEEIINNKRPDEINYIT |
| 3300014664 | YLILNYLPFLTYYYKPNINLIFILRTYIEALIELKNETTIYSYKHNFVKLPNINEEFTYAVTGTLQRITDIDEKDL |
| SEQ ID NO: | IPVKDNSSIILQKDNGLTSKGFYFLICLFLERKYAFSFLSKIEINTAFTDIQNRFFLEVYTQLCCKVPVFNSSNN |
| 4420 | DIILEIFNELNRCPLSVYYVLDKKDKASFRENYKNDRNEDIQASIMKRLESKFAYLTLKFFEETKSLDGISFH |
| | LKLGNIHQKEPHKKEIIGEVRTHHLLKEMKGFGPLAFYKEEEAYSFYTNNSEIESYSPKYRITGNRIGLSLNN |
| | DSSKNYKIIYENVSPDVILSINDLHSLFFYNYLYKQNLINESPKELIEKFMLSFKDFTEDLKTGKLTPVSIEHTI |
| | KKRRKHTEEEIQKLEEAKIELQQKLDPYQLKIKYIPDQSREYLFGYSPHSLEHRIKSKFDRMWKEAIH |
| GCA_ | MTHEPTQKALFGAFLNTAQHNAYLIINEVNEKLGKADVEEGKLDNDAYALHILTNKEIKTLPLKILSRRLL |
| 900113045.1_ | MEGFPFLCALGFSQDATQTDNFEALKTKLQKALKTLNEYRNFYSHYYEQSIDWQKCQFADLDLIREDFIKT |
| IMGtaxon_ | FTTYASEEQVSKSYEEVKKKKEELDKCKKGLSLAKRKKQTWEINDLKQKEEVLRDELLKAQQAHFQLKEK |
| 2636415974_ | KERKENLLKRYPNISGAELNKLIQDKESPYHSFWKKNNPHRFSKKGLVFFICLFLSKEQANLFLSSISGFKRT |
| annotated_ | DASYFWAVRAMYMHHCCHLPQPRLESSDMLLDILNELNRCPKVLYNLLSETNRAYFEKDINYKEGSVLQT |
| assembly_ | DEEGNLVTIQKMLRHQDRLPYFILKYLDETNAFPDLRFQIYLGKLVTDVYKKPKMLEKNELGELVEQDGQ |
| genomic_2 | RLILKEVHAFGKPSDFADKINLPKELEVNEIQDKYGEMKQEELKVGTIVQFSPQYHISSNRIALKLFKTKDG |
| SEQ ID NO: | KFYSEKPDNDKQARLIILNKNWFWF |
| 4421 | |
| UOPM01.1 | MERASFWFEVKKMLKIGCKFFLFLITHYIYKLTLECYLTITMSTFDQSEYLKSEHFQKGVKIESKKHSLIQSI |
| SEQ ID NO: | AEDLRRCPKILFNVITPSGKRQFLPTFGELQETDLIEDHSKIDLATDFEENERLAKPNIRSKNRFSEYALRYID |
| 4422 | EIGLLGNYHFQLDLGSFVLTQYKKNFLGSNVPRKVVDHAMTFSKLKDIVNEDEVRNKISHNVHGLVFEMF |
| | NPHYNIRNNKIAISSKLEYSTVFFNPNHDRKVAIKLRQPQPEAFISIHELPKLLLLDYLSKGKVEELIKNFIQSN |
| | RQKKLNIDFIKKVKSLLPGEDHWTIIERLPDNRFGSGYSDVQLEIISERKRVLNGVLNSYSLNVKQIPTRILDH |
| | WLNIQDSNIDLLFSNRIRSMKSDCLKRLQAFDVNSRHYTGRIPSYTEMAYFLVKDIVSMVISDSKKSKITSFY |
| | FKKLVDCILNYSDPEKRKLFFLIIASELRLLDLGGHPFLGRLDLHNISTTKDFYVSYLQEKGCKMVSQMDSY |
| | TQRMKLVDQSWLFTTFFQRKWNESSGMYKLFVRYPKMDMDIPLKIRRWYKPHSDLQSWLNKTSSSGSSN |
| | KRGKGVDLPANLFDKVICELLRAKLNDLNVAYKPDANYNELLKLWWSSCNDIVQTFYNLERQYFISGEVV |
| | KFHIGTCPNFKDYYSSALEAVFRRNVEERTLEQQKGSVLPDIQITDVEYPFKHTIAETEKKIRILQEQDQMML |
| | LMLRQLMEDDQLFSFSEGDSLLKDK |
| UOPK01.1 | MERASFWFEVKKMLKIGCKFFLFLITHYIYKLTLECYLTITMSTFDQSEYLKSEHFQKGVKIESKKHSLIQSI |
| SEQ ID NO: | AEDLRRCPKILFNVITPSGKRQFLPTFGELQETDLIEDHSKIDLATDFEENERLAKPNIRSKNRFSEYALRYID |
| 4423 | EIGLLGNYHFQLDLGSFVLTQYKKNFLGSNVPRKVVDHAMTFSKLKDIVNEDEVRNKISHNVHGLVFEMF |
| | NPHYNIRNNKIAISSKLEYSTVFFNPNHDRKVAIKLRQPQPEAFISIHELPKLLLLDYLSKGKVEELIKNFIQSN |
| | RQKKLNIDFIKKVKSLLPGEDHWTIIERLPDNRFGSGYSDVQLEIISERKRVLNGVLNSYSLNVKQIPTRILDH |
| | WLNIQDSNIDLLFSNRIRSMKSDCLKRLQAFDVNSRHYTGRIPSYTEMAYFLVKDIVSMVISDSKKSKITSFY |
| | FKKLVDCILNYSDPEKRKLFFLIIASELRLLDLGGHPFLGRLDLHNISTTKDFYVSYLQEKGCKMVSQMDSY |
| | TQRMKLVDQSWLFTTFFQRKWNESSGMYKLFVRYPKMDMDIPLKIRRWYKPHSDLQSWLNKTSSSGSSN |
| | KRGKGVDLPANLFDKVICELLRAKLNDLNVAYKPDANYNELLKLWWSSCNDIVQTFYNLERQYFISGEVV |
| | KFHIGTCPNFKDYYSSALEAVFRRNVEERTLEQQKGSVLPDIQITDVEYPFKHTIAETEKKIRILQEQDQMML |
| | LMLRQLMEDDQLFSFSEGDSLLKDK |
| OGRG01.1 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPGGYDIPSSLNCIYDSAINIIKE |
| 4424 | RFQAEEKEMEHLRRYTRKKGRVVLKTEDDHFYYTLANNNDLSEKGYAFFISMFLERKYSYLFLKKLSGFK |
| | RGDSLQYRLTLEVFTALSTKPPVERLRTTKDTKQDRALDILNELSKIPIELYQTLEPKYREMYNETLQPTDAE |
| | DPYGLPDRSRIRFRSRFEAFALHFLDKQADFK |
| OBVQ01.1 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPGGYDIPSSLNCIYDSAINIIKE |
| 4425 | RFQAEEKEMEHLRRYTRKKGRVVLKTEDDHFYYTLANNNDLSEKGYAFFISMFLERKYSYLFLKKLSGFK |
| | RGDSLQYRLTLEVFTALSTKPPVERLRTTKDTKQDRALDILNELSKIPIELYQTLEPKYREMYNETLQPTDAE |
| | DPYGLPDRSRIRFRSRFEAFALHFLDKQADFK |
| OBVO01.1_2 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPGGYDIPSSLNCIYDSAINIIKE |
| 4426 | RFQAEEKEMEHLRRYTRKKGRVVLKTEDDHFYYTLANNNDLSEKGYAFFISMFLERKYSYLFLKKLSGFK |
| | RGDSLQYRLTLEVFTALSTKPPVERLRTTKDTKQDRALDILNELSKIPIELYQTLEPKYREMYNETLQPTDAE |
| | DPYGLPDRSRIRFRSRFEAFALHFLDKQADFK |
| ORUQ01.1 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPGGYDIPSSLNCIYDSAINIIKE |
| 4427 | RFQAEEKEMEHLRRYTRKKGRVVLKTEDDHFYYTLANNNDLSEKGYAFFISMFLERKYSYLFLKKLSGFK |
| | RGDSLQYRLTLEVFTALSTKPPVERLRTTKDTKQDRALDILNELSKIPIELYQTLEPKYREMYNETLQPTDAE |
| | DPYGLPDRSRIRFRSRFEAFALHFLDKQADFKEIGFYTYLGNYFHNGYQKTRVDRETKDRYINFQLAGFCK |
| | NIQDISAKKLSEALNVKSIDISTDSIPDINSFEPYLVQSTPHYIVNGNNIGIKVLPEGKDTYPTIDEKGAKMPIA |
| | DFWLSKYELPAMLFYTYLRNNNIHKSHCPLSVKDIIERSIHKSTKQKHPEERSELMLRRVMKAIFWTDSKLN |
| | EVERIKSQKSAFGKRQHEILKAGRIAETLVRDMLWLQPSKNNGRDKVTEPNFQAIQVSLAYFGIRRNDLTEI |
| | FTRAGLINSSNPHPFLAQIGTNYTSLIEFYIAYLKERKVYFSRIQKKILQGKLNIQCHPLRDLQREPNKPQEKE |
| | EAIFLPRGLFNEAIINCLKKSKLKHLIESPTREKSPALNVSYLIQNYFRAYFEDQSQEFYAQPRNYRLFDNLSP |
| | NKGKSKSYLSLEQRIKKMEELRPSKIPVAEANKLLEKEDRLYRKNYNEICDNESIIRLYQIQDILLFMMTKEY |
| | LPSDLYNRINKYKLENVKGILNERVSYLIDLNPLKIQGEDIKI |
| UZSP01.1 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPGGYDIPSSLNCIYDSAINIIKE |
| 4428 | RFQAEEKEMEHLRRYTRKKGRVVLKTEDDHFYYTLANNNDLSEKGYAFFISMFLERKYSYLFLKKLSGFK |
| | RGDSLQYRLTLEVFTALSTKPPVERLRTTKDTKQDRALDILNELSKIPIELYQTLEPKYREMYNETLQPTDAE |
| | DPYGLPDRSRIRFRSRFEAFALHFLDKQADFKEIGFYTYLGNYFHNGYQKTRVDRETKDRYINFQLAGFCK |
| | NIQDISAKKLSEALNVKSIDISTD |
| ORTU01.1 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPGGYDIPSSLNCIYDSAINIIKE |
| 4429 | RFQAEEKEMEHLRRYTRKKGRVVLKTEDDHFYYTLANNNDLSEKGYAFFISMFLERKYSYLFLKKLSGFK |
| | RGDSLQYRLTLEVFTALSTKPPVERLRTTKDTKQDRALDILNELSKIPIELYQTLEPKYREMYNETLQPTDAE |
| | DPYGLPDRSRIRFRSRFEAFALHFLDKQADFKEIGFYTYLGNYFHNGYQKTRVDRETKDRYINFQLAGFCK |
| | NIQDISAKKLSEALNVKSIDISTDSIPDINSFEPYLVQSTPHYIVNGNNIGIKVLPEGKDTYPTIDEKGAKMPIV |
| | DFWLSKYELPAMLFYTYLRNNNIHKSHCPLSVKDIIERSIHKSTKQKHPEERSELMLRRVMKAIFWTDSKLN |
| | EVERIKSQKSAFGKRQHEILKAGRIAETLVRDMLWLQPSKNNGRDKVTEPNFQAIQVSLAYFGIRRNDLTEI |
| | FTRAGLINSSNPHPFLAQIGTNYTSLIEFYIAYLKERKVYFSRIQKKILQGKLNIQCHPLRDLQREPNKPQDKE |
| | EAIFLPRGLFNEAIFSR |
| UMHW01.1 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPGGYDIPSSLNCIYDSAINIIKE |
| 4430 | RFQAEEKEMEHLRRYTRKKGRVVLKTEDDHFYYTLANNNDLSEKGYAFFISMFLERKYSYLFLKKLSGFK |
| | RGDSLQYRLTLEVFTALSTKPPVERLRTTKDTKQDRALDILNELSKIPIELYQTLEPKYREMYNETLQPTDAE |
| | DPYGLPDRSRIRFRSRFEAFALHFLDKQADFKEIGFYTYLGNYFHNGYQKTRVDRETKDRYINFQLAGFCK |
| | NIQDISAKKLSEALNVKSIDISTDSIPDINSFEPYLVQSTPHYIVNGNNIGIKVLPEGKDTYPTIDEKGAKMPIV |
| | DFWLSKYELPAMLFYTYLRNNNIHKSHCPLSVKDIIERSIHKSTKQKHPEERSELMLRRVMKAIFWTDSKLN |
| | EVERIKSQKSAFGKRQHEILKAGRIAETLVRDMLWLQPSKNNGRDKVTEPNFQAIQVSLAYFGIRRNDLTEI |
| | FTRAGLINSSNPHPFLAQIGTNYTSLIEFYIAYLKERKVYFSRIQKKILQGKLNIQCHPLRDLQREPNKPQDKE |
| | EAIFLPRGLFNEAIFSR |
| IMG_ | MGAIENKHIFAAYANLAIDGLIKTLNFIAKKLDTQKQLSSWDIKHVITLIDSIFDQNPQNNLEQVVEGYLPWI |
| 3300006464 | KPIIEMKTPKKGERQSDKLCIEYKTIITAFASLLNDVRNYYTHYYHDPICIYPGGYDIPSSLNCIYDSAINIIKE |
| SEQ ID NO: | RFQAEEKEMEHLRRYTRKKGRVVLKTEDDHFYYTLANNNDLSEKGYAFFISMFLERKYSYLFLKKLSGFK |
| 4431 | RGDSLQYRLTLEVFTALSTKPPVERLRTTKDTKQDRALDILNELSKIPIELYQTLEPKYREMYNETLQPTDAE |
| | DPYGLPDRSRIRFRSRFEAFALHFLDKQADFKEIGFYTYLGNYFHNGYQKTRVDRETKDRYINFQLAGFCK |
| | NIQDISAKKLSEALNVKSIDISTDSIPDINSFEPYLVQSTPHYIVNGNNIGIKVLPEGKDTYPTIDEKGAKMPIA |
| | DFWLSKYELPAMLFYTYLRNNNIHKSHCPLSVKDIIERSIHKSTKQKHPEERSELMLRRVMKAIFWTDSKLN |
| | EVERIKSQKSAFGKRQHEILKAGRIAETLVRDMLWLQPSKNNGRDKVTEPNFQAIQVSLAYFGIRRNDLTEI |
| | FTRAGLINSSNPHPFLASSKARRIYSVSFISLPPNKKAPNQIVQDKANRRSISCAKRKL |
| UZRL01.1 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPGGYDIPSSLNCIYDSAINIIKE |
| 4432 | RFQAEEKEIEHLRRYTRKKGRVVLKTEDDHFYYTLVNNNDLSEKGYAFFISMFLERKYSYLFLKKLSGFKR |
| | GDSLQYRLTLEVFTALSTKPPVERLRTTKDTKQDRALDILNELSRIPIELYQTLEPKYREMYNETLQPTDAED |
| | PYGLPDRSRIRFRSRFEAFALHFLDKQADFKEIGFYTYLGNYFHNGYQKTRVDRETKDRYINFQLAGFCKNI |
| | QDISAKKLSEALNVKSIDISTDSIPDINSFEPYLVQSTPHYIVNGNNIGIKVLPEGKDTYPTIDEKGAKMPIADF |
| | WLSKYELPAMLFYTYLRNNNIHKSHCPLSVKDIIERSIHKSTKQKHPEERSELMLRRVMKAIFWTDSKLNEV |
| | ERIKSQKSAFGKRQHEILKAGRIAETLVRDMLWLQPSKNNGRDKVTEPNFQAIQVSLAYFGIRRNDLTEIFT |
| | RAGLINSSNPHPFLAQIGTT |
| OZUY01.1 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPGGYDIPSSLNCIYDSAINIIKE |
| 4433 | RFQAEEKEIEHLRRYTRKKGRVVLKTEDDHFYYTLVNNNDLSEKGYAFFISMFLERKYSYLFLKKLSGFKR |
| | GDSLQYRLTLEVFTALSTKPPVERLRTTKDTKQDRALDILNELSRIPIELYQTLEPKYREMYNETLQPTDAED |
| | PYGLPDRSRIRFRSRFEAFALHFLDKQADFKEIGFYTYLGNYFHNGYQKTRVDRETKDRYINFQLAGFCKNI |
| | QDISAKKLSEALNVKSIDISTDSIPDINSFEPYLVQSTPHYIVNGNNIGIKVLPEGKDTYPTIDEKGAKMPIADF |
| | WLSKYELPAMLFYTYLRNNNIHKSHCPLSVKDIIERSIHKSTKQKHPEERSELMLRRVMKAIFWTDSKLNEV |
| | ERIKSQKSAFGKRQHEILKAGRIAETLVRDMLWLQPSKNNGRDKVTEPNFQAIQVSLAYFGIRRNDLTEIFT |
| | RAGLINSSNPHPFLAQIGTNYTSLIEFYIAYLKERKVYFSRIQKKILQGKLNIQCHPLRDLQREPNKPQDKEEA |
| | IFLPRGLFNEAIINCLKKSKLKQLIESPTREKSPALNVSYLIQNYFRTYFEDQSQEFYAQPRNYRLFDKLSPNK |
| | GKSKSYLSLEQRIKKMEELRPSKIPVAEANKLLEKEDRLYRKNYNEICDNESIIRLYQIQDILLFMMTKEYLP |
| | SDLYNRINKYKLENVKGILNERVSYLIDLNPLKIQGEDIKIKDYGKLFYIHHDTRISSLNKVLSKVKRSNSISS |
| | SCLLYTSPSPRD |
| UZOU01.1 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPGGYDIPSSLNCIYDSAINIIKE |
| 4434 | RFQAEEKEIEHLRRYTRKKGRVVLKTEDDHFYYTLVNNNDLSEKGYAFFISMFLERKYSYLFLKKLSGFKR |
| | GDSLQYRLTLEVFTALSTKPPVERLRTTKDTKQDRALDILNELSRIPIELYQTLEPKYREMYNETLQPTDAED |
| | PYGLPDRSRIRFRSRFEAFALHFLDKQADFKEIGFYTYLGNYFHNGYQKTRVDRETKDRYINFQLAGFCKNI |
| | QDISAKKLSEALNVKSIDISTDSIPDINSFEPYLVQSTPHYIVNGNNIGIKVLPEGKDTYPTIDEKGAKMPIADF |
| | WLSKYELPAMLFYTYLRNNNIHKSHCPLSVKDIIERSIHKSTKQKHPEERSELMLRRVMKAIFWTDSKLNEV |
| | ERIKSQKSAFGKRQHEILKAGRIAETLVRDMLWLQPSKNNGRDKVTEPNFQAIQVSLAYFGIRRNDLTEIFT |
| | RAGLINSSNPHPFLAQIGTNYTSLIEFYIAYLKERKVYFSRIQKKILQGKLNIQCHPLRDLQREPNKPQDKEEA |
| | IFLPRGLFNEAIINCLKKSKLKQLIESPTREKSPALNVSYLIQNYFRTYFEDQSQEFYAQPRNYRLFDKLSPNK |
| | GKSKSYLSLEQRIKKMEELRPSKIPVAEANKLLEKEDRLYRKNYNEICDNESIIRLYQIQDILLFMMTKEYLP |
| | SDLYNRINKYKLENVKGILNERVSYLIDLNPLKIQGEDIKIKDYGKLFYIHHDTRISSL |
| OLEV01.1 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPGGYDIPSSLNCIYDSAINIIKE |
| 4435 | RFQAEEKEIEHLRRYTRKKGRVVLKTEDDHFYYTLVNNNDLSEKGYAFFISMFLERKYSYLFLKKLSGFKR |
| | GDSLQYRLTLEVFTALSTKPPVERLRTTKDTKQDRALDILNELSRIPIELYQTLEPKYREMYNETLQPTDAED |
| | PYGLPDRSRIRFRSRFEAFALHFLDKQADFKEIGFYTYLGNYFHNGYQKTRVDRETKDRYINFQLAGFCKNI |
| | QDISAKKLSEALNVKSIDISTDSIPDINSFEPYLVQSTPHYIVNGNNIGIKVLPEGKDTYPTIDEKGAKMPIADF |
| | WLSKYELPAMLFYTYLRNNNIHKSHCPLSVKDIIERSIHKSTKQKHPEERSELMLRRVMKAIFWTDSKLNEV |
| | ERIKSQKSAFGKRQHEILKAGRIAETLVRDMLWLQPSKNNGRDKVTEPNFQAIQVSLAYFGIRRNDLTEIFT |
| | RAGLINSSNPHPFLAQIGTNYTSLIEFYIAYLKERKVYFSRIQKKILQGKLNIQCHPLRDLQREPNKPQDKEEA |
| | IFLPRGLFNEAIINCLKKSKLKQLIESPTREKSPALNVSYLIQNYFRTYFEDQSQEFYAQPRNYRLFDKLSPNK |
| | GKSKSYLSLEQRIKKMEELRPSKIPVAEANKLL |
| OLEP01.1 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPGGYDIPSSLNCIYDSAINIIKE |
| 4436 | RFQAEEKEIEHLRRYTRKKGRVVLKTEDDHFYYTLVNNNDLSEKGYAFFISMFLERKYSYLFLKKLSGFKR |
| | GDSLQYRLTLEVFTALSTKPPVERLRTTKDTKQDRALDILNELSRIPIELYQTLEPKYREMYNETLQPTDDED |
| | PYGLPDRSRIRFRSRFEAFALHFLDKQADFKEIGFYTYLGNYFHNGYQKTRVDRETKDRYINFQLAGFCKNI |
| | QDISAKKLSEALNVKSIDISTDSIPDINSFEPYLVQSTPHYIVNGNNIGIKVLPEGKDTYPTIDEKGAKMPIADF |
| | WLSKYELPAMLFYTYLRNNNIHKSHCPLSVKDIIERSIHKSTKQKHPEERSELMLRRVMKAIFWTDSKLNEV |
| | ERIKSQKSAFGKRQHEILKAGRIAETLVRDMLWLQPSKNNGRDKVTEPNFQAIQVSLAYFGIRRNDLTEIFT |
| | RAGLINSSNPHPFLAQIGTNYTSLIEFYIAYLKERKVYFSRIQKKILQGKLNIQCHPLRDLQREPNKPQDKEEA |
| | IFLPRGLFNEAIINCLKKSKLKQLIESPTREKSPALNVSYLIQNYFRTYFEDQSQEFYAQPRNYRLFDKLSPNK |
| | GKSKSYLSLEQRIKKMEELRPSKIPVAEANKLLEKE |
| GCA_ | MGAIENKHIFAAYANLAIDGLIKTLNFIAKKLDTQKQLSSWDIKHVITLIDSIFDQNPQNNLEQVVEGYLPWI |
| 003462945.1_ | KPIIEMKTPKKGERQSDKLCIEYKTIITAFASLLNDVRNYYTHYYHDPICIYPGGYDIPSSLNCIYDSAINIIKE |
| ASM346294v1_ | RFQAEEKEMEHLRRYTRKKGRVVLKTEDDHFYYTLANNNDLSEKGYAFFISMFLERKYSYLFLKKLSGFK |
| genomic | RGDSLQYRLTLEVFTALSTKPPVERLRTTKDTKQDRALDILNELSRIPIELYQTLEPKYREMYNETLQPTDAE |
| SEQ ID NO: | DPYGLPDRSRIRFRSRFEAFALHFLDKQADFKEIGFYTYLGNYFHNGYQKTRVDRETKDRYINFQLAGFCK |
| 4437 | NIQDISAKKLSEALNVKSIDISTDSIPDINSFEPYLVQSTPHYIVNGNNIGIKVLPEGKDTYPTIDEKGAKMPIA |
| | DFWLSKYELPAMLFYTYLRNNNIHKSHCPLSVKDIIERSIHKSTKQKHPEERSELMLRRVMKAIFWTDSKLN |
| | EVERIKSQKSAFGKRQHEILKAGRIAETLVRDMLWLQPSKNNGRDKVTEPNFQAIQVSLAYFGIRRNDLTEI |
| | FTRAGLINSSNPHPFLAQIGTNYTSLIEFYIAYLKERKVYFSRIQKKILQGKLNIQCHPLRDLQREPNKPQDKE |
| | EAIFLPRGLFNEAIINCLKKSKLKQLIESPTREKSPALNVSYLIQNYFRTYFEDQSQEFYAQPRNYRLFDKLSP |
| | NKGKSKSYLSLEQRIKKMEELRPSKIPVAEANKLLEKEDRLYRKNYNEICDNESIIRLYQIQDILLFMMTKEY |
| | LPSDLYNRINKYKLENVKGIFK |
| UYDU01.1 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPGGYDIPSSLNCIYDSAINIIKE |
| 4438 | RFQAEEKEMEHLRRYTRKKGRVVLKTEDDHFYYTLANNNDLSEKGYAFFISMFLERKYSYLFLKKLSGFK |
| | RGDSLQYRLTLEVFTALSTKPPVERLRTTKDTKQDRALDILNELSRIPIELYQTLEPKYREMYNETLQPTDAE |
| | DPYGLPDRSRIRFRSRFEAFALHFLDKQADFKEIGFYTYLGNYFHNGYQKTRVDRETKDRYINFQLAGFCK |
| | NIQDISAKKLSEALNVKSIDISTDSIPDINSFEPYLVQSTPHYIVNGNNIGIKVLPEGKDTYPTIDEKGAKMPIA |
| | DFWLSKYELPAMLFYTYLRNNNIHKSHCPLSVKDIIERSIHKSTKQKHPEERSELMLRRVMKAIFWTDSKLN |
| | EVERIKSQKSAFGKRQHEILKAGRIAETLVRDMLWLQPSKNNGRDKVTEPNFQAIQVSLAYFGIRRNDLTEI |
| | FTRAGLINSSNPHPFLAQIGTNYTSLIEFYIAYLKERKVYFSRIQKKILQGKLNIQCHPLRDLQREPNKPQEKE |
| | EAIFLPRGLFNEAIINCLKKSKLKQLIESPTREKSPALNVSYLILNYFRTYFEDQSQEFYAQPRNYRLFDKLSP |
| | NKGKSKSYLSLEQRIKKMEELRPSKIPVAEANKLLEKEDRLYRKNYNEICDNESI |
| OPWW01.1 | MGAIENKHIFAAYANLAIDGLIKTLNFIAKKLDTQKQLSSWDIKHVITLIDSIFDQNPQNNLEQIVEGYLPWI |
| SEQ ID NO: | KPIIEMKTPKKGERQSDKLCIEYKTIITAFASLLNDVRNYYTHYYHDPICIYPRGYDIPSSLNCIYDSAINIIKE |
| 4439 | RFQAEEKEMEHLRRYTRKKGRVVLKTEDDHFYYTLVNNNGLSEKGYAFFISMFLERKYSYLFLKKLSGFK |
| | RGDSLQYRLTLEVFTALSTKPPVERLRTTKDTKQDRALDILNELSKIPIELYQTLEPKYREMYNETLQPTDAE |
| | DPYGLPDRSRIRFRSRFETFALHFLDKQADFKEIGFYTYLGNYFHNGYQKTRVDRETKDRYINFQLAGFCK |
| | NIQDISAKKLSEALNVKSIDISTDSIPDINSFEPYLVQSTPHYIVNGNNIGIKVLPEGKDTYPTIDEKGAKMPIA |
| | DFWLSKYELPAMLFYTYLRNNNIHKSHCPLSVKDIIERSIHKSTKQKHPEERSELMLRRVMKAIFWTDSKLN |
| | EVERIKSQKSAFGKRQHEILKAGRIAETLVRDMLWLQPSKNNGRDKVTEPNFQAIQVSLAYFGIRRNDLTEI |
| | FTRAGLINSSNPHPFLAQIGTNYTSLIEFYIAYLKERKVYFSRIQKKILQGKLNIQCHPLRDLQREPNKPQDKE |
| | EAIFLPRGLFNEAIINCLKKSKLKQLIESPTREKSPALNVSYLIQNYFRTYFEDQSQEFYAQPRNYRLFDKLSP |
| | NKGKSKSYLSLEQRIKKMEELRPSKIPVAEANKLLEKEDRLYRKNYNEICDNESIIRLYQIQDILLFMMTKEY |
| | LPSDLYNRINKYKLENVKGILNERVSYLIDLNLLKIQGEDIKIKDYGKLFYIHHDTRISSLNKVLSKVKR |
| ORLB01.1 | MGAIENKHIFAAYANLAIDGLIKTLNFIAKKLDTQKQLSSWDIKHVITLIDSIFDQNPQNNLEQIVEGYLPWI |
| SEQ ID NO: | KPIIEMKTPKKGERQSDKLCIEYKTIITAFASLLNDVRNYYTHYYHDPICIYPRGYDIPSSLNCIYDSAINIIKE |
| 4440 | RFQAEEKEMEHLRRYTRKKGRVVLKTEDDHFYYTLVNNNGLSEKGYAFFISMFLERKYSYLFLKKLSGFK |
| | RGDSLQYRLTLEVFTALSTKPPVERLRTTKDTKQDRALDILNELSKIPIELYQTLEPKYREMYNETLQPTDAE |
| | DPYGLPDRSRIRFRSRFETFALHFLDKQADFKEIGFYTYLGNYFHNGYQKTRVDRETKDRYINFQLAGFCK |
| | NIQDISAKKLSEALNVKSIDISTDSIPDINSFEPYLVQSTPHYIVNGNNIGIKVLPEGKDTYPTIDEKGAKMPIA |
| | DFWLSKYELPAMLFYTYLRNNNIHKSHCPLSVKDIIERSIHKSTKQKHPEERSELMLRRVMKAIFWTDSKLN |
| | EVERIKSQKSAFGKRQHEILKAGRIAETLVRDMLWLQPSKNNGRDKVTEPNFQAIQVSLAYFGIRRNDLTEI |
| | FTRAGLINSSNPHPFLAQIGTNYTSLIEFYIAYLKERKVYFSRIQKKILQGKLNIQCHPLRDLQREPNKPQDKE |
| | EAIFLPRGLFNEAIINCLKKSKLKQLIESPTREKSPALNVSYLIQNYFRTYFEDQSQEFYAQPRNYRLFDKLSP |
| | NKGKSKSYLSLEQRIKKMEELRPSKIPVAEANKLLEKEDRLYRKNYNEICDNESIIRLYQIQDILLFMMTKEY |
| | LPSDLYNRIN |
| UZST01.1 | MGAIENKHIFAAYANLAIDGLIKTLNFIAKKLDTQKQLSSWDIKHVITLIDSIFDQNPQNNLEQIVEGYLPWI |
| SEQ ID NO: | KPIIEMKTPKKGERQSDKLCIEYKTIITAFASLLNDVRNYYTHYYHDPICIYPRGYDIPSSLNCIYDSAINIIKE |
| 4441 | RFQAEEKEMEHLRRYTRKKGRVVLKTEDDHFYYTLVNNNGLSEKGYAFFISMFLERKYSYLFLKKLSGFK |
| | RGDSLQYRLTLEVFTALSTKPPVERLRTTKDTKQDRALDILNELSKIPIELYQTLEPKYREMYNETLQPTDAE |
| | DPYGLPDRSRIRFRSRFETFALHFLDKQADFKEIGFYTYLGNYFHNGYQKTRVDRETKDRYINFQLAGFCK |
| | NIQDISAKKLSEALNVKSIDISTDSIPDINSFEPYLVQSTPHYIVNGNNIGIKVLPEGKDTYPTIDEKGAKMPIA |
| | DFWLSKYELPAMLFYTYLRNNNIHKSHCPLSVKDIIERSIHKSTKQKHPEERSELMLRRVMKAIFWTDSKLN |
| | EVERIKSQKSAFGKRQHEILKAGRIAETLVRDMLWLQPSKNNGRDKVTEPNFQAIQVSLAYFGIRRNDLTEI |
| | FTRAGLINSSNPHPFLAQIGTNYTSLIEFYIAYLKERKVYFSRIQKKILQGKLNIQCHPLRDLQREPNKPQDKE |
| | EAIFLPRGLFNEAIINCLKKSKLKQLIESPTREKSPALNVSYLIQNYFRTYFEDQSQEFYAQPRNYRLFDKLSP |
| | NKGKSKSYLSLEQRIKKMEELRPSKIPVAEANKLLEKEDRLYRKNYNEICDNESIIRLYQIQDILLFMMTKEY |
| | LPSDLYNRINKYKLENVKGILNERVSYLIDLNLLKIQGEDIKIKDYGKLFYIHHDTRISSLNKVLSKVK |
| OYVX01.1 | MGAIENKHIFAAYANLAIDGLIKTLNFIAKKLDTQKQLSSWDIKHVITLIDSIFDQNPQNNLEQIVEGYLPWI |
| SEQ ID NO: | KPIIEMKTPKKGERQSDKLCIEYKTIITAFASLLNDVRNYYTHYYHDPICIYPRGYDIPSSLNCIYDSAINIIKE |
| 4442 | RFQAEEKEMEHLRRYTRKKGRVVLKTEDDHFYYTLVNNNGLSEKGYAFFISMFLERKYSYLFLKKLSGFK |
| | RGDSLQYRLTLEVFTALSTKPPVERLRTTKDTKQDRALDILNELSKIPIELYQTLEPKYREMYNETLQPTDAE |
| | DPYGLPDRSRIRFRSRFETFALHFLDKQADFKEIGFYTYLGNYFHNGYQKTRVDRETKDRYINFQLAGFCK |
| | NIQDISAKKLSEALNVKSIDISTDSIPDINSFEPYLVQSTPHYIVNGNNIGIKVLPEGKDTYPTIDEKGAKMPIA |
| | DFWLSKYELPAMLFYTYLRNNNIHKSHCPLSVKDIIERSIHKSTKQKHPEERSELMLRRVMKAIFWTDSKLN |
| | EVERIKSQKSAFGKRQHEILKAGRIAETLVRDMLWLQPSKNNGRDKVTEPNFQAIQVSLAYFGIRRNDLTEI |
| | FTRAGLINSSNPHPFLAQIGTNYTSLIEFYIAYLKERKVYFSRIQKKILQGKLNIQCHPLRDLQREPNKPQDKE |
| | EAIFLPRGLFNEAIINCLKKSKLKQLIESPTREKSPALNVSYLIQNYFRTYFEDQSQEFYAQPRNYRLFDKLSP |
| | NKGKSKSYLSLEQRIKKMEELRPSKIPVAEANKLLEKEDRLYRKNYNEICDNESIIRLYQIQDILLFMMTKEY |
| | LPSDLYNRINKYKLENVKGILNERVSYLIDLNLLKIQGEDIKIKDYGKLF |
| ORTO01.1 | MGAIENKHIFAAYANLAIDGLIKTLNFIAKKLDTQKQLSSWDIKHVITLIDSIFDQNPQNNLEQIVEGYLPWI |
| SEQ ID NO: | KPIIEMKTPKKGERQSDKLCIEYKTIITAFASLLNDVRNYYTHYYHDPICIYPRGYDIPSSLNCIYDSAINIIKE |
| 4443 | RFQAEEKEMEHLRRYTRKKGRVVLKTEDDHFYYTLVNNNGLSEKGYAFFISMFLERKYSYLFLKKLSGFK |
| | RGDSLQYRLTLEVFTALSTKPPVERLRTTKDTKQDRALDILNELSKIPIELYQTLEPKYREMYNETLQPTDAE |
| | DPYGLPDRSRIRFRSRFETFALHFLDKQADFKEIGFYTYLGNYFHNGYQKTRVDRETKDRYINFQLAGFCK |
| | NIQDISAKKLSEALNVKSIDISTDSIPDINSFEPYLVQSTPHYIVNGNNIGIKVLPEGKDTYPTIDEKGAKMPIA |
| | DFWLSKYELPAMLFYTYLRNNNIHKSHCPLSVKDIIERSIHKSTKQKHPEERSELMLRRVMKAIFWTDSKLN |
| | EVERIKSQKSAFGKRQHEILKAGRIAETLVRDMLWLQPSKNNGRDKVTEPNFQAIQVSLAYFGIRRNDLTEI |
| | FTRAGLINSSNPHPFLAQIGTNYTSLIEFYIAYLKERKVYFSRIQKKILQGKLNIQCHPLRDLQREPNKPQDKE |
| | EAIFLPRGLFNEAIINCLKKSKLKQLIESPTREKSPALNVSYLIQNYFRTYFEDQSQEFYAQPRNYRLFDKLSP |
| | NKGKSKSYLSLEQRIKKMEELRPSKIPVAEANKLLEKEDRLYRKNYNEICDNESIIRLYQIQDILLFMMTKEY |
| | LPSDLYNRINKYKLENVKGILNE |
| OHBF01.1 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPRGYDIPSSLNCIYDSAINIIKE |
| 4444 | RFQAEEKEMEHLRRYTRKKGRVVLKTEDDHFYYTLANNNDLSEKGYAFFISMFLERKYSYLFLKKLSGFK |
| | RGDSLQYRLTLEVFTALSTKPPVERLRTTKDTKQDRALDILNELSKIPIELYQTLEPKYREMYNETLQPTDAE |
| | DPYGLPDRSRIRFRSRFETFALHFLDKQADFKEIGFYTYLGNYFHNGYQKTRVDRETKDRYINFQLAGFCK |
| | NIQDISAKKLSEALNVKSIDISTDSIPDINSFEPYLVQSTPHYIVNGNNIGIKVLPEGKDTYPTIDEKGAKMPIA |
| | DFWLSKYELPAMLFYTYLRNNNIHKSHCPLSVKDIIERSIHKSTKQKHPEERSELMLRRVMKAIFWTDSKLN |
| | EVERIKSQKSAFGKRQHEILKAGRIAETLVRDMLWLQPSKNNGRDKVTEPNFQAIQVSLAYFGIRRNDLTEI |
| | FTRAGLINSSNPHPFLAQIGTNYTSLIEFYIAYLKERKVYFSRIQKKILQGKLNIQCHPLRDLQREPNKPQDKE |
| | EAIFLPRGLFNEAIINCLKKSKLKQLIESPTREKSPALNVSYLIQNYFRTYFEDQSQEFYAQPRNYRLFDKLSP |
| | NKGKSKSYLSLEQRIKKMEELRPSKIPVAEANKLLEKEDRL |
| OXOU01.1 | MGAIKNKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPGGYDIPSSLNCIYDSAINIIKE |
| 4445 | RFQAEEKEMEHLRRYTRKKGRVVLKTEDDHFYYTLANNNDLSEKGYAFFISMFLERKYSYLFLKKLSGFK |
| | RGDSLQYRLTLEVFTALSTKPPVERLRTTKDTKQDRALDILNELSKIPIELYQTLEPKYREMYNETLQPTDAE |
| | DPYGLPDRSRIRFRSRFEAFALHFLDKQADFKEIGFYTYLGNYFHNGYQKTRVDRETKDRYINFQLAGFCK |
| | NIQDISAKKLSEALNVKSIDISTDSIPDINSFEPYLVQSTPHYIVNGNNIGIKVLPEGKDTYPTIDEKGAKMPIA |
| | DFWLSKYELPAMLFYTYLRNNNIHKSHCPLSVKDIIERSIHKSTKQKHPEERSELMLRRVMKAIFWTDSKLN |
| | EVERIKSQKSAFGKRQHEILKAGRIAETLVRDMLWLQPSKNNGRDKVTEPNFQAIQVSLAYFGIRRNDLTEI |
| | FTRAGLINSSNPHPFLAQIGTNYTSLIEFYIAYLKERKVYFSRIQKKILQGKLNIQCHPLRDLQREPNKPQDKE |
| | EAIFLPRGLFNEAIINCLKKSKLKQLIESPTREKSPALNVSYLIQNYFRTYFEDQSQEFYAQPRNYRLFDKLSP |
| | NKGKSKSYLSLEQRIKKMEELRPSKIPVAEANKLLEKEDRLYRKNYNEICDNESIIRLYQIQDILLFMMTKEY |
| | LPSDCLLYTSPSPRDA |
| ULRQ01.1 | LDTQKQLSSWDIKHVITLIDSIFDQNPQNNLEQVVEGYLPWIKPIIEMKTPKKGERQSDKLCIEYKTIITAFAS |
| SEQ ID NO: | LLNDVRNYYTHYYHDPICIYPGGYDIPSSLNCIYDSAINIIKERFQAEEKEMEHLRRYTRKKGRVVLKTEDD |
| 4446 | HFYYTLANNNDLSEKGYAFFISMFLERKYSYLFLKKLSGFKRGDSLQYRLTLEVFTALSTKPPVERLRTTKD |
| | TKQDRALDILNELSRIPIELYQTLEPKYREMYNETLQPTDAEDPYGLPDRSRIRFRSRFEAFALHFLDKQADF |
| | KEIGFYTYLGNYFHNGYQKTRVDRETKDRYINFQLAGFCKNIQDISAKKLSEALNVKSIDISTDSIPDINSFEP |
| | YLVQSTPHYIVNGNNIGIKVLPEGKDTYPTIDEKGAKMPIADFWLSKYELPAMLFYTYLRNNNIHKSHCPLS |
| | VKDIIERSIHKSTKQKHPEERSELMLRRVMKAIFWTDSKLNEVERIKSQKSAFGKRQHEILKAGRIAETLVR |
| | DMLWLQPSKNNGRDKVTEPNFQAIQVSLAYFGIRRNDLTEIFTRAGLINSSNPHPFLAQIGTNYTSLIEFYIA |
| | YLKERKVYFSRIQKKILQGKLNIQCHPLRDLQREPNKPQDKEEAIFLPRGLFNEAIINCL |
| OJZH01.1 | MGAIENKHIFAAYANLAIDGLIKTLNFIAKKLDTQKQLSSWDIKHVITLIDSIFDQNPQNNLEQIVEGYLPWI |
| SEQ ID NO: | KPIIEMKTPKKGERQSDKLCIEYKTIITAFASLLNDVRNYYTHYYHDPICIYPGGYDIPSSLNCIYDSAINIIKE |
| 4447 | RFQAEEKEMEHLRRYTRKKGRVVLKTEDDHFYYTLANNNDLSEKGYAFFISMFLERKYSYLFLKKLSGFK |
| | RGDSLQYRLTLEVFTALSTKPPVERLRTTKDTKQDRALDILNELSKIPIELYQTLEPKYREMYNETLQPTDAE |
| | DPYGLPDRSRIRFRSRFETFALHFLDKQADFKEIGFYTYLGNYFHNGYQKTRVDRETKDRYINFQLAGFCK |
| | NIQDISAKKLSEALNVKSIDISTDSIPDINSFEPYLVQSMPHYIVNGNNIGIKVLPEGKDTYPTIDEKGAKMPIA |
| | DFWLSKYELPAMLFYTYLRNNNIHKSHCPLSVKDIIERSIHKSTKQKHPEERSELMLRRVMKAIFWTDSKLN |
| | EVERIKSQKSAFGKRQHEILKAGRIAEMLVRDMLWLQPSKNNGRDKVTEPNFQAIQVSLAYFGIRRNDLTEI |
| | FTRAGLINSSNPHPFLAQIGTNYTSLIEFYIAYLKERKVYFSRIQKKILQGKLNIQCHPLRDLQREPNKPQEKE |
| | EAIFLPRGLFNEAIINCLKKSKLKQLIESPTREKSPALNVSYLIQNYFRTYFEDQSQEFYAQPRNYRLFDKLSP |
| | NKGKSKSYLSLEQRIKKMEELRPSKIPVAEANKLLEKEDRLYRKNYNEICDNESIIRLYQIQDILLFMMTKEY |
| | LPSDLYNRINKYKLENVKG |
| UXJA01.1 | MGAIENKHIFAAYANLAIDGLIKTLNFIAKKLDTQKQLSSWDIKHVITLIDSIFDQNPQNNLEQIVEGYLPWI |
| SEQ ID NO: | KPIIEMKTPKKGERQSDKLCIEYKTIITAFASLLNDVRNYYTHYYHDPICIYPGGYDIPSSLNCIYDSAINIIKE |
| 4448 | RFQAEEKEMEHLRRYTRKKGRVVLKTEDDHFYYTLVNNNDLSEKGYAFFISMFLERKYSYLFLKKLSGFK |
| | RGDSLQYRLTLEVFTALSTKPPVERLRTTKDTKQDRALDILNELSKIPIELYQTLEPKYREMYNETLQPTDAE |
| | DPYGLPDRSRIRFRGRFEAFALHFLDKQADFKEIGFYTYLGNYFHNGYQKTRVDRETKDRYINFQLAGFCK |
| | NIQDISAKKLSEVLNVKSIDISTDSIPDINSFEPYLVQSTPHYIVNGNNIGIKVLPEGKDTYPTIDEKGAKMPIA |
| | DFWLSKYELPAMLFYTYLRNNNIHKSHCPLSVKDIIERSIHKSTKQKHPEERSELMLRRVMKAIFWTDSKLN |
| | EVERIKSQKSAFGKRQHEILKAGRIAETLVRDMLWLQPSKNNGRDKVTEPNFQAIQVSLAYFGIRRNDLTEI |
| | FTRAGLINSSNPHPFLAQIGTNYTSLIEFYIAYLKERKVYFSRIQKKILQGKLNIQCHPLRDLQREPNKPQDKE |
| | EVTFLPRGLFNEAIINCLKKSKIKQLIESPTREKSPALNVSYLIQNYFKTYFEDQSDEFYAQPRNYCS |
| IMG_ | MGAIKNKHIFAAYANLAIDGLIKTLNFIAKKLDTQKQLSSWDIKHVITLIDSIFDQNPQNNLEQVVEGYLPWI |
| 3300014947 | KPIIEMKTPKKGERQSDKLCIEYKTIITAFASLLNDVRNYYTHYYHDPICIYPGGYDIPSSLNCIYDSAINIIKE |
| SEQ ID NO: | RFQAEEKEMEHLRRYTRKKGRVVLKTEDDHFYYTLANNNDLSEKGYAFFISMFLERKYSYLFLKKLSGFK |
| 4449 | RGDSLQYRLTLEVFTALSTKPPVERLRTTKDTKQDRALDILNELSKIPIELYQTLEPKYREMYNETLQPTDAE |
| | DPYGLPDRSRIRFRSRFEAFALHFLDKQADFKEIGFYTYLGNYFHNGYQKTRVDRETKDRYINFQLAGFCK |
| | NIQDISAKKLSEALNVKSIDISTDSIPDINSFEPYLVQSTPHYIVNGNNIGIKVLPEGKDTYPTIDEKGAKMPIA |
| | DFWLSKYELPAMLFYTYLRNNNIHKSHCPLSVKDIIERSIHKSTKQKHPEERSELMLRRVMKAIFWTDSKLN |
| | EVERIKSQKSAFGKRQHEILKAGRIAETLVRDMLWLQPSKNNGRDKVTEPNFQAIQVSLAYFGIRRNDLTEI |
| | FTRAGLINSSNPHPFLAQIGTNYTSLIEFYIAYLKERKXXXXIFHEYKRKFSKGN |
| UZJI01.1 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPGGYDIPSSLNCIYDSAINIIKE |
| 4450 | RFQAEEKEMEHLRRYTRKKGRVVLKTEDDHFYYTLANNNDLSEKGYAFFISMFLERKYSYLFLKKLSGFK |
| | RGDSLQYRLTLEVFTALSTKPPVERLRTTKDTKQDRALDILNELSRIPIELYQTLEPKYREMYNETLQPTDAE |
| | DPYGLPDRSRIRFRSRFEAFALHFLDKQADFKEIGFYTYLGNYFHNGYQKTRVDRETKDRYINFQSQAFVK |
| | TSKISPQRNYRRH |
| OZEI01.1 | MGAIENKHIFAAYANLAIDGLIKTLNFIAKKLDTQKQLSSWDIKHVITLIDSIFDQNPQNNLEQIVEGYLPWI |
| SEQ ID NO: | KPIIEMKTPKKGERQSDKLCIEYKTIITAFASLLNDVRNYYTHYYHDPICIYPRGYDIPSSLNCIYDSAINIIKE |
| 4451 | RFQAEEKEMEHLRRYTRKKGRVVLKTEDDHFYYTLVNNNGLSEKGYAFFISMFLERKYSYLFLKKLSGFK |
| | RGDSLQYRLTLEVFTALSTKPPVERLRTTKDTKQDRALDILNELSKIPIELYQTLEPKYREMYNETLQPTDAE |
| | DPYGLPDRSRIRFRSRFETFALHFLDKQADFKEIGFYTYLGNYFHNGYQKTRVDRETKDRYINFQLAGFCK |
| | NIQDISAKKLSEALNVKSIDISTDSIPDINSFEPYLVQSTPHYIVNGNNIGIKVLPEGKDTYPTIDEKGAKMPIA |
| | DFWLSKYELPAMLFYTYLRNNNCLLYTSPS |
| OJMI01.1 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPRGYDIPSSLNCIYDSAINIIKE |
| 4452 | RFQAEEKEMEHLRNYTLVNNNGLSEKGYAFFISKFLERKYSYLFLKKLSGFKRGDSLQYRLTLEVFTALST |
| | KPPVERLRTTKDTKQDRALDILNELSRIPIELYQTLEPKYREMYNETLQPTDAEDPYGLPDRSRIRFRSRFEA |
| | FALHFLDKQADFKEIGFYTYLGNYFHNGYQKTRVDRETKDRYINFQLAGFCKNIQDISAKKLSEALNVKSI |
| | DISTDSIPDINSFEPYLVQSTPHYIVNGNNIGIKVLPEGKDTYPTIDEKGAKMPIADFWLSKYELPAMLFYTYL |
| | RNNNIHKSHCPLSVKDIIERSIHKSTKQKHPEERSELMLRRVMKAIFWTDSKLNEVERIKSQKSAFGKRQHEI |
| | LKAGRIAETLVRDMLWLQPSKNNGRDKVTEPNFQAIQVSLAYFGIRRNDLTEIFTRAGLINSSNPHPFLAQIG |
| | TNYTSLIEFYIAYLKERKVYFSRIQKKILQGKLNIQCHPLRDLQREPNKPQEKEEAIFLPRGLFNEAIINCLKK |
| | SKLKHLIESPTREKSPALNVSYLIHNYFRAYFEDQSQEFYAQPRNYRLFDKLSPNKGKSKSYLSLEQRIKKM |
| | EELRTKAIQDSCCRS |
| CDZK01.1 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPRGYDIPSSLNCIYDSAINIIKE |
| 4453 | RFQAEEKEMEHLRNYTLVNNNGLSEKGYAFFISKFLERKYSYLFLKKLSGFKRGDSLQYRLTLEVFTALST |
| | KPPVERLRTTKDTKQDRALDILNELSRIPIELYQTLEPKYREMYNETLQPTDAEDPYGLPDRSRIRFRSRFEA |
| | FALHFLDKQADFKEIGFYTYLGNYFHNGYQKTRVDRETKDRYINFQLAGFCKNIQDISAKKLSEALNVKSI |
| | DISTDSIPDINSFEPYLVQSTPHYIVNGNNIGIKVLPEGKDTYPTIDEKGAKMPIADFWLSKYELPAMLFYTYL |
| | RNNNIHKSHCPLSVKDIIERSIHKSTKQKHPEERSELMLRRVMKAIFWTDSKLNEVERIKSQKSAFGKRQHEI |
| | LKAGRIAETLVRDMLWLQPSKNNGRDKVTEPNFQAIQVSLAYFGIRRNDLTEIFTRAGLINSSNPHPFLAQIG |
| | TNYTSLIEFYIAYLKERKVYFSRIQKKILQGKLNIQCHPLRDLQREPNKPQEKEEAIFLPRGLFNEAIINCLKK |
| | SKLKHLIESPTREKSPALNVSYLIHNYFRAYFEDQSQEFYAQPRNYRLFDKLSPNKGKSKSYLSLEQRIKKM |
| | EELRTKAIQDSCCRS |
| CDYT01.1 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPRGYDIPSSLNCIYDSAINIIKE |
| 4454 | RFQAEEKEMEHLRNYTLVNNNGLSEKGYAFFISKFLERKYSYLFLKKLSGFKRGDSLQYRLTLEVFTALST |
| | KPPVERLRTTKDTKQDRALDILNELSRIPIELYQTLEPKYREMYNETLQPTDAEDPYGLPDRSRIRFRSRFEA |
| | FALHFLDKQADFKEIGFYTYLGNYFHNGYQKTRVDRETKDRYINFQLAGFCKNIQDISAKKLSEALNVKSI |
| | DISTDSIPDINSFEPYLVQSTPHYIVNGNNIGIKVLPEGKDTYPTIDEKGAKMPIADFWLSKYELPAMLFYTYL |
| | RNNNIHKSHCPLSVKDIIERSIHKSTKQKHPEERSELMLRRVMKAIFWTDSKLNEVERIKSQKSAFGKRQHEI |
| | LKAGRIAETLVRDMLWLQPSKNNGRDKVTEPNFQAIQVSLAYFGIRRNDLTEIFTRAGLINSSNPHPFLAQIG |
| | TNYTSLIEFYIAYLKERKVYFSRIQKKILQGKLNIQCHPLRDLQREPNKPQEKEEAIFLPRGLFNEAIINCLKK |
| | SKLKHLIESPTREKSPALNVSYLIHNYFRAYFEDQSQEFYAQPRNYRLFDKLSPNKGKSKSYLSLEQRIKKM |
| | EELRTKAIQDSCCRS |
| OHGO01.1 | MGAIENKHIFAAYANLAIDGLIKTLNFIAKKLDTQKQLSSWDIKHVITLIDSIFDQNPQNNLEQVVEGYLPWI |
| SEQ ID NO: | KPIIEMKTPKKGERQSDKLCIEYKTIITAFASLLNDVRNYYTHYYHDPICIYPGGYDIPSSLNCIYDSAINIIKE |
| 4455 | RFQAEEKEMKHLRNYTLVNNNGLSEKGYAFFISKFLERKYSYLFLKKLSGFKRGDSLQYRLTLEVFTALST |
| | KPPVERLRTTKDTKQDRALDILNELSKIPIELYQTLEPKYREMYNETLQPTDAEDPYGLPDRSRIRFRSRFEA |
| | FALHFLDKQADFKEIGFYTYLGNYFHNGYQKTRVDRETKDRYINFQLAGFCKNIQDISAKKLSEALNVKSI |
| | DISTDSIPDINSFEPYLVQSTPHYIVNGNNIGIKVLPEGKDTYPTIDEKGAKMPIADFWLSKYELPAMLFYTYL |
| | RNNNIHKSHCPLSVKDIIERSIHKSTKQKHPEERSELMLRRVMKAIFWTDSKLNEVERIKSQKSAFGKRQHEI |
| | LKAGRIAETLVRDMLWLQPSKNNGRDKVTEPNFQAIQVSLAYFGIRRNDLTEIFTRAGLINSSNPHPFLAQIG |
| | TNYTSLIEFYIAYLKERKVYFSRIQKKILQGKLNIQCHPLRDLQREPNKPQEKEEAIFLPRGLFNEAIINCLKK |
| OGRG01.1_2 | MPIADFWLSKYELPAMLFYTYLRNNNIHKSHCPLSVKDIIERSIHKSTKQKHPEERSELMLRRVMKAIFWTD |
| SEQ ID NO: | SKLNEVERIKSQKSAFGKRQHEILKAGRIAETLVRDMLWLQPSKNNGRDKVTEPNFQAIQVSLAYFGIRRN |
| 4456 | DLTEIFTRAGLINSSNPHPFLAQIGTNYTSLIEFYIAYLKERKVYFSRIQKKILQGKLNIQCHPLRDLQREPNKP |
| | QEKEEAIFLPRGLFNEAIINCLKKSKLKHLIESPTREKSPALNVSYLIQNYFRAYFEDQSQEFYAQPRNYRLFD |
| | NLSPNKGKSKSYLSLEQRIKKMEELRPSKIPVAEANKLLEKEDRLYRKNYNEICDNESIIRLYQIQDILLFMM |
| | TKEYLPSDLYNRINKYKLENVKGILNERVSYLIDLNPLKIQGEDIKIKDYGKLFYIHHDTRISSLNKVLSKVK |
| | RNNSISSSVKIQPYENYKRECLDFEEAQIQIIPIIHSFEIAMVSMFPDLKKATPGNYYDFNELITEYEKRTKQKI |
| | DSSFLIKTRNMFLHDKYEAECIKEISDDFVYAKKIIAEFKMKIENIKLEDFSNDSSA |
| OBVQ01.1_2 | MPIADFWLSKYELPAMLFYTYLRNNNIHKSHCPLSVKDIIERSIHKSTKQKHPEERSELMLRRVMKAIFWTD |
| SEQ ID NO: | SKLNEVERIKSQKSAFGKRQHEILKAGRIAETLVRDMLWLQPSKNNGRDKVTEPNFQAIQVSLAYFGIRRN |
| 4457 | DLTEIFTRAGLINSSNPHPFLAQIGTNYTSLIEFYIAYLKERKVYFSRIQKKILQGKLNIQCHPLRDLQREPNKP |
| | QEKEEAIFLPRGLFNEAIINCLKKSKLKHLIESPTREKSPALNVSYLIQNYFRAYFEDQSQEFYAQPRNYRLFD |
| | NLSPNKGKSKSYLSLEQRIKKMEELRPSKIPVAEANKLLEKEDRLYRKNYNEICDNESIIRLYQIQDILLFMM |
| | TKEYLPSDLYNRINKYKLENVKGILNERVSYLIDLNPLKIQGEDIKIKDYGKLFYIHHDTRISSLNKVLSKVK |
| | RNNSISSSVKIQPYENYKRECLDFEEAQIQIIPIIHSFEIAMVSMFPDLKKATPGNYYDFNELITEYEKRTKQKI |
| | DSSFLIKTRNMFLHDKYEAECIKEISDDFVYAKKIIAEFKMKIENIKLEDFSNDSSA |
| OBVO01.1 | MPIADFWLSKYELPAMLFYTYLRNNNIHKSHCPLSVKDIIERSIHKSTKQKHPEERSELMLRRVMKAIFWTD |
| SEQ ID NO: | SKLNEVERIKSQKSAFGKRQHEILKAGRIAETLVRDMLWLQPSKNNGRDKVTEPNFQAIQVSLAYFGIRRN |
| 4458 | DLTEIFTRAGLINSSNPHPFLAQIGTNYTSLIEFYIAYLKERKVYFSRIQKKILQGKLNIQCHPLRDLQREPNKP |
| | QEKEEAIFLPRGLFNEAIINCLKKSKLKHLIESPTREKSPALNVSYLIQNYFRAYFEDQSQEFYAQPRNYRLFD |
| | NLSPNKGKSKSYLSLEQRIKKMEELRPSKIPVAEANKLLEKEDRLYRKNYNEICDNESIIRLYQIQDILLFMM |
| | TKEYLPSDLYNRINKYKLENVKGILNERVSYLIDLNPLKIQGEDIKIKDYGKLFYIHHDTRISSLNKVLSKVK |
| | RNNSISSSVKIQPYENYKRECLDFEEAQIQIIPIIHSFEIAMVSMFPDLKKATPGNYYDFNELITEYEKRTKQKI |
| | DSSFLIKTRNMFLHDKYEAECIKEISDDFVYAKKIIAEFKMKIENIKLEDFSNDSSA |
| IMG_ | LKKSKLKQLIESPTREKSPALNVSYLIQNYFRTYFEDQSQEFYAQPRNYRLFDKLSPNKGKSKSYLSLEQRIK |
| 3300014947_2 | KMEELRPSKIPVAEANKLLEKEDRLYRKNYNEICDNESIIRLYQIQDILLFMMTKEYLPSDLYNRINKYKLEN |
| SEQ ID NO: | VKGILNERVSYLIDLNPLKIQGEDIKIKDYGKLFYIHHDTRISSLNKVLSKVKRNNSISSSVKIQPYENYKREC |
| 4459 | LDFEEAQIQIIPIIHSFEIAMVSMFPDLKKATPGNYYDFNELITEYEKRTKQKIDSSFLIKTRNMFLHDKYEAE |
| | CIKEISDDFVYAKKIIAEFKMKIENIKLEDLSNDSSA |
| UZJI01.1_2 | MPIADFWLSKYELPAMLFYTYLRNNNIHKSHCPLSVKDIIERSIHKSTKQKHPEERSELMLRRVMKAIFWTD |
| SEQ ID NO: | SKLNEVERIKSQKSAFGKRQHEILKAGRIAETLVRDMLWLQPSKNNGRDKVTEPNFQAIQVSLAYFGIRRN |
| 4460 | DLTEIFTRAGLINSSNPHPFLAQIGTNYTSLIEFYIAYLKERKVYFSRIQKKILQGKLNIQCHPLRDLQREPNKP |
| | QDKEEAIFLPRGLFNEAIINCLKKSKLKQLIESPTREKSPALNVSYLIQNYFRTYFEDQSQEFYAQPRNYRLFD |
| | KLSPNKGKSKSYLSLEQRIKKMEELRPSKIPVAEANKLLEKEDRLYRKNYNEICDNESIIRLYQIQDILLFMM |
| | TKEYLPSDLYNRINKYKLENVKGILNERVSYLIDLNPLKIQGEDIKIKDYGKLFYIHHDTRINSLNKVLSKVK |
| | RNNSISSSVKIQPYENYKRECLDFEEAQIQIIPIIHSFEITMVSMFPDLKKATPGNYYDFNELITEYEKRTKQKI |
| | DSSFLIKTRNMFLHDKYEAECIKEISDDFVYAKKIIAEFKMKIENIKLEDLSNDSSA |
| UXPW01.1 | LQKAIFWTDSKLNEVERIKSQKSAFGKRQHEILKAGRIAETLVRDMLWLQPSKNNGRDKVTEPNFQAIQVS |
| SEQ ID NO: | LAYFGIRRNDLTEIFTRAGLINSSNPHPFLAQIGTNYTSLIEFYIAYLKERKVYFSRIQKKILQGKLNIQCHPLR |
| 4461 | DLQREPNKPQDKEEAIFLPRGLFNEAIINCLKKSKLKQLIESPTREKSPALNVSYLIQNYFRTYFEDQSQEFYA |
| | QPRNYRLFDKLSPNKGKSKSYLSLEQRIKKMEELRPSKIPVAEANKLLEKEDRLYRKNYNEICDNESIIRLYQ |
| | IQDILLFMMTKEYLPSDLYNRINKYKLENVKGILNERVSYLIDLNLLKIQGEDIKIKDYGKLFYIHHDTRISSL |
| | NKVLSKVKRNNSISSSVKIQPYENYKRECLDFEEAQIQIIPIIHSFEIAMVSMFPDLKKATPGNYYDFNELITE |
| | YEKRTKQKIDSSFLIKTRNMFLHDKYEAECIKEISDDFVYAKKIIAEFKMKIENIKLEDLSNDSSA |
| OJMG01.1 | MDSKYILGSYLNMANDNFINTIHLLAKKLKTPKGEDQNLDQVVSYIPRIFDNSNQVPETEQIVEFYFPWLEA |
| SEQ ID NO: | LKNANGFGKSELPELKIFYKDVLCSFAKNLKDLRDNYTHYIHKTISYHPIIYKKEGNTLQSSLPKALLQIYDA |
| 4462 | SIKLAKERFLADENAVTHLRRYKIVGKKVVRKTEADHFMYLLEKDHLLTEKGIAFFTSLFLNRKYGYLMLK |
| | QLKGFKQGETLTYRLTLETFLAYSNIKPVERLKADKYSDVAFIMDLLGEISKIPKELYHILPETYILQYKNKL |
| | HIELNEDISLEAYCSRGRNRFDQLALTYLDRLEDFKQIGFYTYLGNYIHNGYMKARIDGTEQKRYLSEKMY |
| | GCCKNIYRDLSAIVAKQYNIEVKDSTETSYMLPNDFQPHVIRAYPHYVINNNNIGIRLLDEGESGFPILENRG |
| | TKKM |
| mgm4491477.3 | MEEANRYIYGAYFNMARDNFLNTIKLLADKMKLGAISGFGKDGNEVNDINKLFGDKNTYVNIENVVEFYF |
| SEQ ID NO: | PWIKALEGRFSLDKGDRNLNDMKMFYKSVLTAFFTAVDSLRNKYTHYSHKDLNIREIKIECTLGGKDYCIG |
| 4463 | LLNTLDCIYDSAVNLLKLRFMAGEDEVAHLRRCKAVNKMVVVRTEKDGFYYRLSDNGGVTEKGVIFIAS |
| | MFLNRKYGFLFLKQLEGFKRSDEKRYRLTLETFLAFSNIKPVDRLKSDKLDRASLGLDMLNELTKIPKELSE |
| | TLSVDCLYKYLASDGEDDLRSRIRYQDRFVPLALEFISQSDEFKDFRFYTYVGNYVYKGYIKRLIDGTDKER |
| | YLSDRLCGFYKSVNDASSDAIAQKYGVEIKDSNEPDYMLPDSFRPHVLRATPHFVINNNNIGIKICGNDCLP |
| | VVNGKGVESPEPDYWLSIYELPAMLFYAYLREKNGKLLKDYKSIRELIEDVEKKADEKNDRDKGALMARH |
| | IDKEIIWTQTKLDEVKRLEEKKVAAYGKKGRVVLKSGRMADLLAHDMVRLQPATKGSDKITGANFQALQ |
| | VSLAYFKRDILADVFSRAMLTTGNHRHPFLYRIDVSHCSSLRDFYVAYLGERRKYFEDVAKKITKNKLNTP |
| | CHILRRLQREGSGEEAGKDVKPKFLPRGIFTGSIKSCLEKSALNINIRNARNDVKPAINAAYLILMYYKEIEK |
| | GEFQGFYGEKRRYDILEEGKSLDLDERKKALASIKPAKIDVSEANMPMSKEEHLMRKXXXXXXXXXXXX |
| | XXXXXXXXXXXXXXXXXXXXXXQ |
| OJMG01.1_2 | MSKYDLPALLFYAYLRSDSRFASKCKKTIDEILRGYLSKSKDKKPKQAEKASVLMLRRIDKAIIWTQTKLNE |
| SEQ ID NO: | AEKQRDNKKSFKIGEKADILAHDMLWLQPAKESKDKVSGANFRALQTSLAFFRRNELDDIFKRSFLIGGNN |
| 4464 | PHPFLSRIKINTMPSLFDFYLAYMRERLNYWEKIKLKLLKGHINVSCHPLRKLNHGKPVDQDNKKDEQPIFL |
| | PRGIFNDVIKNCLQKTKLGIYLKDQGAEKRPWNVAYQILKFHEIINDDDIQEFYKQPRKYAILDENHYLTLE |
| | ERTDRLKDLKPECIEVSKANDILEKEDYLLRKSYNQVCDNESAIRLYQVQDILLFMIAQRLCNEIILGDKQEK |
| | KEKVETSFSLTLKNLSKKFDQPVHFEIKMNNVNLFSDTDIPVKNFGKMLRLKKDARFISYGKLFKGQKQNI |
| | NYNDYCKEEEYFDICRIQMVKLCHELEEKLLEKGIITESPNSGYYPFADLVQRIINQGVVISSSDVKFILEARN |
| | MFLHNEYKQCCVNSINSIDSFIAEKVYNLFKQKMDNILGDLESIKPS |
| IMG_ | MAFDKPRPKRRQARSDFYITDKAIMGGYFNLAQLNFFKTLMEIFTKAGIDVSKIKQDNSPKYLMILIKKLTH |
| 3300028886 | DDEKIDDKKWADALDLSNECLLKLQQLLYKHFPFLGPVMGGEASYNIYKLKDGHPEVKVANDVMRGVKL |
| SEQ ID NO: | EDCLTVLRHFALCLNDCRNFYTHFNPYNSIDAQKEQYLSQNKVAVWLDKVKDASRRIVKQNNQLTSQDM |
| 4465 | EFLTGIDHMKPQDKVDEFGNIMTDRRGYTIKEYVEYEDYYFRIRGKRYLVDAAGAKLADQEPRNALSDFG |
| | VVFLCTFFLESDQSRRMLDELRLFETGPYDGSRDGDEFKNDILREILLFYRARIPRGKRLDPMDDTTLLAMD |
| | MLNELRKCPMPLYDVLSREGQRFFEDEVKRPNDLTPEVAKRLRSSDRFPYLALRYIDLNKCFDNIRFQVQL |
| | GKFRYKFYDKTTIDGEQVVRGLQKEVNAYGRLQDVERYRQEKYADMLQQTELVETGEEDITIANFIPDTPQ |
| | SSPYLSDRTASYNIHNNRIGLFWNMPGEQEVLTGDEKMYLPDLNVDDNGKADVFLPAPKASLSVRDLPAL |
| | VFYLYLQNQHPDLMPAESIIQQKYNALVRFFEDVSTGRLQPVKGINELKAAIDRYEYLTIHEIPEKLRDYLA |
| | GVAGTEDDDDCADRLDCYAMGILEKRYRRVAARIDQLKEARKMVGDKMNRYGKKSY |
| LXOW01.2 | MQQHNPKRRKAQSSFLISEKSIMGGYFNIARLNLYKTVITIFAQVGIKGDYQEDKIDRVLDALYKNLAGKSE |
| SEQ ID NO: | ELSKEQSQWKRLNQLKNEQIVKLQRLLFKHFPVLGPIMASEASYKIYKAELCAKDAEEKARNDKAELKKV |
| 4466 | RKSNVINDEQLMRGVGIDECLDVLATMASCLTDCRNFYSHYVPYNNKEEQKIQYGRQAKIARWLDKVIVA |
| | SRRIDKQRNSLSTGEMEFLTGIDHYFPKEKVDENGRVIKDNRGWAVKEFVEYPDYYFRIKGERQLIDTSGV |
| | TLTGEKAMDALTDFGIVFFCTLFLQKTYAKMMQEELALYESGPYNGTVKGQEENDAKKNAILREMLSIYRI |
| | RIPRGKRLDSQDDTTTLAMDMLNELRKCPMSLYDVLGQEGQRFFEDEVQHPNEQTPEKAKRLRATDRFPY |
| | FALRYIDLKKDIFTRIRFQVDLGNYRFKFYNKKTIDGLEEVRSLQKEINGY |
| IMG_ | MTNYHHNNQGSKSPNGQGQNRGSKYGKSGESRRQRRRQTQNFAISLTGKNVFGAYFNMARTNFVKTINYI |
| 3300031994 | LPIAGVRGKYEENKIDKMLHALFLIQAGRGAELTPEQREWRQKLILNPEQQERLKSLLFRHFPMLKPMMAD |
| SEQ ID NO: | FIDHKIYKNKKKSTIQTDDEAFELLRGVSLADCLDMVVLMAETLTECRNFYTHADPYNSAVDLAKQYQHQ |
| 4467 | AAIAKKLDKLVVASRRVLKEMENLSVEEVEFLTGVDHMAQIPRKDEAGQVIRDEKGRKQMKFVEYDDFY |
| | FKISGTRPVQGLSMTGPDGQPTTVDSQLPALSDFGLLFFCVLFLSKPYAKLFIEEARL |
| IMG_ | MTNYHHNNQGSKSPNGQGQNRGSQYGKSGESRRQRRRQTQNFAISLTGKNVFGAYFNMARTNFVKTINYI |
| 3300028805 | LPIAGVRGKYEENKIDKMLHALFLIQAGRGAELTPEQREWRQKLILNPEQQERLKSLLFRHFPMLKPMMAD |
| SEQ ID NO: | FIDHKIYKNKKKSTIQTDDEAFELLRGVSLADCLDMVVLMAETLTECRNFYTHADPYNSAVDLAKQYQHQ |
| 4468 | AAIAKKLDKLVVASRRVLKEMENLSVEEVEFLTGVDHMAQVPRKDEAGQVIRDEKGRKQMKFVEYDDFY |
| | FKISGTRPVQGLSMTGIDGQPTTVDSQLPALSDFGLLFFCVLFLSKPYAKLFIEEARLFEFSPFTEVENLVIRE |
| | MLSIYRIRTPRLHRIDSREDKAALSMDIFGELRRCPMELYNLLDKETDQPFFHDVVKHPNDYTPEVSKRQRH |
| | TDRFPHLALRYVDATKLFERIRFQLQLGAFRYKFYDKKNCIDGRPRVRRIQKEINGYGRLQEVEDKRFEKW |
| | GDLIQKREER |
| IMG_ | MTNYHHNNQGSKRPNGQGQKLGSQQGKSVESRPRRRRQSQDFAISLTGKNVFGAYFNMARTNFVKTINYI |
| 3300032030 | LPIAGVRGKYEENKIDKMLHALFLIQAGRGAELTPEQREWRQKLILNPEQQERLKSLLFRHFPMLKPMMAD |
| SEQ ID NO: | FIDHKIYKNKKKSTIQTDDEAFGLLRGVSLADCLDMVLLMAETLTECRNFYTHADPYNSAVDLAKQYQHQ |
| 4469 | AAIAKKLDKLVVASRRVLKERENLSVEEVEFLTGVDHMAQIPRKDEAGQVIRDEKGRKQMKFVEYDDFYF |
| | KISGTRPVQGLSMTGPDGQPTTVDSQLPALSDFGLLFFCVLFLSKPYAKLFIEEARLFEFSPFTEVENLVMRE |
| | MLSIYRIRTPRLHRIDSREDKAALSMDIFGELRRCPMELYNLLDKETDQPFFHDVVKHPNDYTPEVSKRQRH |
| | TDRFPHLALRYVDATKLFERIRFQLQLGAFRFRFYDKKNCIDGRPRVRRIQKEINGYGRLQEVEDKRFEKW |
| | GDLIQKREEREVKLEHEDMVLDLDQFLQDTADSIPYITDRRPAYNIHAGRIGLFWERSRNPKDFKYFEDGM |
| | YIPQLIVSEDLRAPISMPEPLCSLSVHDLPAMLFYEYLRGQQEGRKFKSAEQIIIDCEGDFRRFFASVADGSLK |
| | PFAREKELREYLSVNFPNLRMADIPEKIRLYLCGQPLRHNNEEETARQRLVRLTLEHLEEREQKIAHRLEHY |
| | QEDRKKIGEKDNKIGKKDHADVRHGALARYIAQSLMLWQPSIDGIGHGKLTGVNYNALTAYLATFGTPQP |
| | EEENFTPRTLLQVLQAANLVEGENPHPFINKVLARGNRNIEELYLHYLDEELNHIRACRQSLQNDPSDAAL |
| mgm4547164.3_ | MGKEHKGNNAPKNRNKVANNSQQPRKVRRLQKTNFRISLSGKHVFGAYFNMARTNFIKTINYILPIAGVR |
| 5 | GNYSESQINKLLQAMFLIQTGRNGELTKEQKQWEKKLRLNPEQQTKLQQLLFKHFPVLGPMMADVADHK |
| SEQ ID NO: | VYLNKKKSNVQTEDEAFAQLKGVSLADCLEMIYLMAETLTECRNFYTHKSPYNTPSQLAMQYLHQEMIA |
| 4470 | KKLDKVVVASRRILKDREGFSVNEVEFLTGIDHLHQETVKDEFGNVKMKGGKVMKTFVEYDDFYFKISGK |
| | RLVKGYTVTVKDDKPVNVDTMLPALSDFGLLYFCVIFLSKPYAKLFIDEVRLFEFSPFSDNENMIMSEMLSI |
| | YRIRTPRLHKIDSRDSKATLAMDIFGELRRCPIELYDLLDKNSGQPFFHDEVKXXXXXXXXXXXXXXXXXX |
| | XXXXXXXXXXXXXXXXXXXXXXXXXXXXXXXXXXXXXXXXXXXXXXXXXXXXXXXXXXXXXXXX |
| | XXXXXXXXXXXXXXXXXXXXNFS |
| IMG_ | MGKENKGNNAPKTQNKTANNSQQQKKVRRLQKTSFRISLTGKHVFGAYFNMARTNFIKTINYILPIAGVRG |
| 3300028805_3 | NYSENQINKMLRALFLIQAGRNGELTTEQKQWEKKLRLNPEQKTRLQKLLFKHFPVLSPMMADVADHKA |
| SEQ ID NO: | YLNKKKSNVQTEDEAFEQLKGISLSDCLEIICLMAETLTECRNFYTHKDPYNKPSLLAAQYQHQEMIAKKL |
| 4471 | DKVIVASRRILKDREGLSVNEVEFLTGIDHLHQEVVKDEFGNVKKKDGKVMKTFVEYDDFYFKISGKRLV |
| | KGFTVTVKDDKPVNVDTMLPALSDFGLLYFCVLFLSKPYAKLFIDEVRLFEFSPFDDNENMIMSEMLSIYRI |
| | RTPRLHKIDSRDNKATLAMDIFGELRRCPIELYDLLDKNTGQPFFHDEVKRPNSHTPEVSKRLRYNDRFPTL |
| | ALRYIDETELFNRIRFQLQLGAFRYKFYDKECIDGRVRVRRIQKDINGYGRLQEVADKRLDKWGDLIQKRE |
| | EQSVKLEHEELYLDLDQFQQDTADSTPYVTDRRPAYNIHANRIGMYWEDSQNPKLFEVFDENKMYIPELK |
| | VSEDMKAPVKMPEPRCALSIYDLSAMLFYEYLREQEDENIPSAEQIIIDYESDYRRFFKAVAEGSLKPFQRTK |
| | EFREYLKKEYPRLHLADIPEKLQSYLCSHGLSF |
| IMG_ | MGKGNKGNEVKIQQPKKKRRIQKTNFTISLTGKHVFGAYFNMARTNFIKTINYILPIAGVRGNYSENQINN |
| 3300028887 | MLHALFLIHAGRNSELSKEQKQWEKKLRLNLEQQTKLQKLLFKHFPVLGPMMADVADHKAFLNKKKSKV |
| SEQ ID NO: | QTEDEAFVQLKGVSLSDCLEMIHLMAITLTECRNFYTHKSPYNTPSQLASQYQHQEQIAKKLDKVVVASRR |
| 4472 | ILKDREGLSINEVEFLTGIDHLHQEIEKDQFGNIVKKNGKVLKTFVEYDDFYFKIFGKRLVKGLTVAVKDND |
| | PVNVDTMLPALSDFGLLYFCVLFLSKPYAKLFIDETRLFEFSPFNDNENMILSEMLSIYRIRTPRLHKIDSRDN |
| | KAALAMDIFGELRCCPIELYDLLDKNTGQSFFHDEVKRPNSHTPEVSKRLRYDDRFPTLALRYIDETELFKRI |
| | RFQIQLGAFRYRFFDKEDCIDGRVRVRSIQKEINGYGRLQEVADKRLEKWGDMLQKREERSVKLEHEELYL |
| | DLDQFQEDTANSTPYVTDRRPSYNIHANRIGLYWEDSQNPKQFKVFDENGMYIPKLIVTEDEKAPINMPAP |
| | RCALSVYDLPAMLFYEYLREQQKGNVQAAEQIIIDYENDYRKFFKAVAEGTLKPFQKTKELREYLEENYPK |
| | LRMSDIPEKIQLYLTSKGLTHNNKPETVRERMIRLINQHLEEREKNVQRRLEHYQEDRKMVGEKENKYGK |
| | KGFADVRHGALARYLTQSMMEWQPSKDGKGYDKLTGLNYNVLTAYLATFGTPQTVEEGFTPKSLEQVLT |
| | KAHIIGGSNPHPFMNKVLSLGSRNIEELYLH |
| IMG_ | MDKKQNHNIVGGQMSASTQPHSNQRRIQSTDFSIGLTGKHVYGAYFNMARTNFVKTVSYIMEIVGIRGKYS |
| 3300001395 | ESQLNNVLQALYLIRAGQSDKLTAVQKTWKKNLRLTVEQQTLFQRLLFKHFPVLNPIMADTANYRAYLKK |
| SEQ ID NO: | ENKRKSTVQSEDETFEQLKGISLADCLEMLVLMGDTLTECRNFYTHLDPYNPPEELEKQYKHQALIAIKLN |
| 4473 | KVIEASRRVLKEREGLTTGEVEFLTGIDHLMQVDKKDEHGNKIYQKNGRPQKTFVEYDDFYFKVSGTRSIQ |
| | GISHPALSDFGLLYLCVLFLSKHYAKLLIEESRLFEFSPFNDNENLILQEMLSIYRIRTPRPKRIDSHDDKATLA |
| | MDIFGELRRCPIVLYDLLDKEKGQPFFHDEVKRPNDHTPEVFKRIRFDDRFPHLALRYIDMAELFKRIRFQL |
| | QLGSFRYKFYDKLCADGQIRVRRIQKDINGYGRLQEVADKRWDFWGDLIQKREELPVKLEHEEVFINLDQF |
| | VQDTADSMPYITDRRPSYN |
| IMG_ | MGKNYYSKNGNGSNKNAKVQKAPRLTNEPFTIREDDKKIYGAYFNMALDNFFKTIAYIFNVLDIKQFVRT |
| 3300028591 | KYNGEYVEVPMFSEESLHIILKYYSKFFGGTLKSDKLKKNVKKLSRLSSKDEEQEQKYEEDLDELIQSLQLT |
| SEQ ID NO: | NEQQQKFQQMLFRHFPFLGPIMADYASYSIYQQASKDVSDKEYIKKRKKEIMNSYDSLRGVTLSQCLEELS |
| 4474 | KMADCLTDCRNKYTHFKPYNSLETQKTQLELQFMIAKKLDKLLAASRRLTKQNIAITTEEMEFITGIDHYE |
| | NVNKQFIEREDFYFNPKGKGMAVIESTDANGAHQQSSSTYDAFSPFGIAYFCILFLSKTYARLFIDEINLFAG |
| | SPFNDSENAIMREIMSLYRVRTPRGKRLDSKATDSTLGMDMLNELRKCPMELYETLSQEGRRFFEDEVKRQ |
| | NDHTPEVVKRLRSTDRFPYLAMRYIDETQMFDDIRFQVRLGSYRFRFYKKIDCVDGVDRVRRIQKEINGFG |
| | RLQDIENERKTNWEAQMQDANYKSVKLEHEDLYLDLRQFPKDTESQQPYITDRRAEYNIHNNRIGLYWNR |
| | ETDTPEYLDDAKCFLPKLETTGDAGKRKAQIIQPAPLCTLSVRELPAMLFYQWLCDNYKTDMPHDSAELLI |
| | KKKYDSLVRLFTAIKNGTFSYKTTEEATVKYLKNKFKLTLTDVPQKLRMYIVGKETHPLQRLIEVTFDGYE |
| | DNSGKHKGKLEQRREKIERRLEKYKDDRKKIGDKTNTY |
| mgm4547164.3_ | MEKSENKQHSQGFPFPHKHVRRPQPLKVDQDNKYVLGGYFSLGLNNFYKTVLLVFAKAGIPIVGKKGTILY |
| 4 | EEEKIGQVLNTLYKCCLNPKPDFEPSEEKWVPFFKLNVNQQMKLHKLLFKHFPILSPIMADEAAYKANKRK |
| SEQ ID NO: | KSKVTDTFSMTLGVSLSDCLKVIGTIAQGLVDCRNADVHFDPYNSLEDLAKQYMVQQDIVRYLVKALVAS |
| 4475 | RRLDKEQNNIETEKMEFLTGYATKKSLEGYIQKWGFYPKYEQQIKKDKDGNPVYVEVTDKDGNPKKDKN |
| | GNPIYKQQLDRRTGKRMCDRDGNPIYEVQKVMVERSDFFYKIGGETTIEKNGKVYSTLTGFGLCYFCTIFLS |
| | KPQARQMLQDIRLFEHSPYPEELNXXXXXXXXXXXXXXXXXXXXXXXXXXXXXXXXXXXXXXXXXXX |
| | XXXXXXXXXXXXXXXXXXXXXXXXXXXXXXXXXXXXXXXXXXXXXXXXXXXXXXXXXXXXXXXX |
| | XXXXXXXXXXXXXXXXXXXXXXXXXXXXXXXXXXXFMTC |
| IMG_ | MEKSNKKANTQPPKKVQEPKALVVNESNKYVLGGYFSLGLNNFYKTILLVFAKTGIKVMSGNGNILYSEE |
| 3300028591_2 | KIGQVLNTLFKSTLSPRPKFEAFEQAWAVNFKLTANQQVTLQKLLFHHFPVLGPIMADEEAYKVIKTKKSN |
| SEQ ID NO: | VTDTYSMTLGVTLSECLKAISIIAQGLVDCRNTDVHYHPYNSLDDLARQYHVQSDIVRYLNKALVASRRID |
| 4476 | KKRNSIETVKMEFLTGYANVDALNKYLAKWHFYPKYDQMSKRDADGNILYVDATDKQGNPLFDKGGNP |
| | KYKQERDRSGKPLYNQDGSPKYEEQKIMVERNDFFYRIGGESTIEKNAETFSTLTGFGLAYFCTLFLSKPQA |
| | KQMLADIKLFERSPYPQELNDIIRDMLSIYRLRSPKGKKLEGGDNQVTLALDILNELRKCPKELYDVLSPEG |
| | QAFFEDEVKRPNERTPEVVKRFRSKDRFSFLALRYIDEMGVFDNIRFQVQLGKLRFKFYPKTCINGEEDVRS |
| | LQKEINGYGKLHEIELERKSKYGPLLQVSTEKSVKIEHEDMYLDLLQFERDKADSQPYITDSKTFYNIYNNRI |
| | GLFWKELEYADSKNNQQVVKPQKGDYLPSLLDVKEGKAPVDMPAPMAMLSVYELPALIFYHYLRSQQKD |
| | IVNPTAEDIIIDKYYSLKRFFMDVCIGTLAPFEKKKLLVETLSSQYGLGINEIPKKLKDYLTGKNINIESKKLK |
| | LTQDILATRLKKAIRRRDGYKDDRKKIGDKENRYGKDSYVDVRHGS |
| mgm4547164.3_ | MSTFKFINKFACGAYFNSARDNFHRAMLDILQQIGVSKHYTETELDDRLAEILNVLKGEPSPISEEDAKKIIA |
| 9 | NQTMFRQLLFRRFPILGPVISDVLHYTHRQKEKIIMKELIDERVHDLIMQNEAEEDYYLSNEQMVLQAEKEI |
| SEQ ID NO: | KEALKTKKKKIIVDDCGDRDATCAECLDAILLLARCLTDCRNYFTHYLPYNPNEKLHNMYGRQCKAATW |
| 4477 | LNPVFTASRRLDKRRNRLTSAQLEFITNHNYKAKTDENGRKVKDENGKDIYIKDHSYYFSIIGESFIAKRNG |
| | DEYVQKDIENDSSAFGNCENNALSDFGLMYLCCCFLTRSQAQQFAEKAKLFANSPENMTERDFQYVLNVS |
| | AEAQPNSIQLEELNRLKTKLRVDNLDLSKEKDRAAFTALQNDRSYWQKTSENILLQEMLSIYRVRLPRGKR |
| | LDKQDNATTVALDMLNELRRCPKELFDILPTEGQQAFAAPVNNEEGETENSVARKRFTDRFPYLALRAIDE |
| | ANLLPSIRFQVQLGYYRFAFYNKTCIDGSSQLRRLGKALNGFGRLSAMESRRKTEWAPEDSESETQQPNRF |
| | QRKIYTPTRLEDGKTVLDLLRPVEDKVGNEPYITDTAASYNIYNNRIGLYWEEQNGGRKNDDILEFPELRTK |
| | DTDKVGTKRPEVPQKAPLCTLSVRDLPALLFLLHINGYNSKAVEDVIVNKYKGLLKFFSDISRXXXXXXXX |
| | XXXXXXXXXXXXXXXXTRFSKVIT |
| IMG_ | MATFRFQNKYVCGAYYNMAQNNFRLAILHVLSRIGINRKDEERAIPELLDTIYGFLTGDTEHFNDTQILQKK |
| 3300031853 | VLTLRYDQQVKLRELLFRQFPILKPIIASETHKSLQEQKEKLSEMEEYIHEFEKQLKRLYRSKKGKASQEQK |
| SEQ ID NO: | EINEQIKYLKTKKSKYLDEYDTLLFSKSHDADLQLCLKILKTMAWCLYDLRDFYTHYDPYNTPESLKVKYL |
| 4478 | RQVTVANWLSQVFAASRTIDKERNSITTEEMNFLNEAKKQGNDKKWREDPDYYFAIKGQNLLSSSFIDADS |
| | PVYNDYLDELYQKYRKKRLAWMKEQRDEALERDDEEEYGFWVVEMEKLDNPERIAKIKSKICPLALSDFG |
| | VLYFCATFLPRNYTLLMADHAEVMKNSPYSMTAEELEARRNACKTDEEREKIDPTDTPRNNILREMLCIYR |
| | VRLPKGKRLDKKDTKGLLTLDILNELRKCPKEVYEQLSKEGKDFFISRVSSASHNAPDIVKRIRFGDRFPYL |
| | ALRAIDESDVFKRIRFQVRLGSYRFYFYNKTCIDGSTQLRRWDKEINGFGRLQDMEALRKSL |
| IMG_ | MNNSVNSFALVNESSGRGKYVAGTYFEIAIHNFFKTIDFVLRRVNIRKSDKQWADGFAHLGNWTDERMGK |
| 3300026539_2 | VLEQLALAEMSPTQLSRLSRLLYHHFPFFSPIMADAADHQVYLRVNEIKEIEQEITESDKKIKKNPVNKELV |
| SEQ ID NO: | QSINEARIRLKAAHNSLERQLASTTAKEVLAKLAVMAYAMNFYRNQYSHKCHFETLNEKTLQEENEQNLA |
| 4479 | FWLEVIFKGARSIILDRKEHSQEDTKFLTQDGNLHYNVNKSKKSTRNPNFYFSPGKKNGQKWLITDFGRYY |
| | FCSLFLQRSDAIEFGKNVGLYTDSPFKLSNEERNKLQLEEKHRALEEQKIVDKEGDGHKVNPRIISNTESVQ |
| | NTIIQEMLDVYRLRIPREGRIDAMMNEGTLIMDILNELRRCPKSVYETFSPADKKKFNKVGTNPDGSKSEMK |
| | LIRYHDRFPYLALRMIDQTNAIGDIRFHLRLGLFRYRFYNKKTISGELVNPVRTIQKEVNGFGRWQDVEEGR |
| | KSLYGKYFQNRIINDDGLEQPVPDSLQSLPYITDWHASYNIHACRIGMAWNLSQMEDALYLPPLTFDDGNN |
| | RNRKAPLDMPAPMCYMSIYDIPALLFYNYL |
| IMG_ | MEKNNKEGKSQSFYKNKNWGKELKQRQIRKFVENMLNYSLPITNTKETSGKSILGAYANVAFDNFDKTLQ |
| 3300031853_2 | YIYKKVGLKVNGTNQVAVLEKILDAYKKEKEWYRSHPNEKKPGKKSQYLLTSEQNEKMKTLLFHHFSVL |
| SEQ ID NO: | APILGSMKNAEIAKIHNEIKKDEENKSLTEEKSAEIIKKVKDVVTSAHIDSCLKVLVNLSKSLHYCRNLHSHY |
| 4480 | RAYNNRENQINMFKNFATTAGYLTNALKASAIICQTNAGNKAKQYEFVTGEYHYMKDKKEYSNYYYRIK |
| | GKRNTIKASDKIEPDQYDAISDYGLIYLTSLFLSKSDTELMLDQLEVFKNSPFKDEFTMEKAVLTSIMAVYRI |
| | NIPKGKRMKMEDDNVQLCLDMLNELQKCPQELYDVISDKGKDSFKREQTEP |
| IMG_ | MADYIFDYIDPDKTKRYIYGTYIEMAFHNFFITLQHIYKCVKGVYPAMQEEDFGRDENTIFIFDDLTNAEDQ |
| 3300028886_3 | ERIKLLLNKHFPFLKVIEDDFNETDKITIIKKCWDFLKSLRNAVEHDTETTKSIFANNKTDILRWLRTDFGCG |
| SEQ ID NO: | KDVKKRGIAIAAKKEMKDRFFLPRDPKSVFYFMNNCNPESGNFAFRSLVIFVSLFLEGKYTYQFITNSELKS |
| 4481 | NFFYKEKNRAGVVNQIEFRKDDFVNLYRALSIYNINLPAVKYDAQFDKTNILGLDIINELQKCPNELYEHIS |
| | KEDQNKFRVQNSDNADYPDEIFLKRFQDRFATLVLRYIDTQKLFKDIRFQVSLGKYRFKFYDKQCIDSDSA |
| | DRVRILQKELKTFGRLDDMEQKRRTEWADILRISDIENPSEADTADTQPYITDQNARYNIDKHTPKIPLWWD |
| | GDCSLPVTKGQVNLGQKCQSIVPKAFLSVYDLPAMMFLHLLGGNPEALIKEYYNNYIKFFTDIRDGKLTPD |
| | TFSEKDFAQTYKIKLCDVPKQLQNFLLRKKPSVPERYQNMPLRLVKKASLNDFQNATLKVIDEKIEKVISEP |
| | QKLK |
| IMG_ | MADYIFDYIDPDKTKRYIYGTYIEMAFHNFFITLQHIYKCVKGVYPAMQEEDFGRDENTIFIFDDLTNAEDQ |
| 3300032007 | ERIKLLLNKHFPFLKVIEDDFNETDKITIIKKCWDFLKSLRNAVEHDTETTKSIFANNKTDILRWLRTDFGCG |
| SEQ ID NO: | KDVKKRGIAIAAKKEMKDRFFLPRDPKSVFYFMNNCNPESGNFAFRSLVIFVSLFLEGKYTYQFITNSELKS |
| 4482 | NFFYKEKNRAGVVNQIEFRKDDFVNLYRALSIYNINLPAVKYDAQFDKTNILGLDIINELQKCPNELYEHIS |
| | KEDQNKFRVQNSDNADYPDEIFLKRFQDRFATLVLRYIDTQKLFKDIRFQVSLGKYRFKFYDKQCIDSDSA |
| | DRVRILQKELKTFGRLDDMEQKRRTEWADILRISDIENPSEADTADTQPYITDQNARYNIDKHTPKIPLWWD |
| | GDCSLPVTKGQVNLGQKCQSIVPKAFLSVYDLPAMMFLHLLGGNPEALIKEYYNNYIKFFTDIRDGKLTPD |
| | TFSEKDFAQTYKIKLCDVPKQLQNFLLRKKPSVPERYQNMPLRLVKKASLNDFQNATLKVIDEKIEKVISEP |
| | QKLK |
| mgm4547164.3_ | LYATYIEMAFHNMFLNIKHIYGVVFGRDIMAEAKANYEALNPEKKWDEDFANEFLVWKPMFEAFNNGNV |
| 10 | EEKQKVGEMLSRHFPLLVPFTDFTNHESNYKNLTIVDILRRLSQVLRVLRNLYSHYRIELFENQKKVYLDNE |
| SEQ ID NO: | YLIIRCTMNSYMGARRVTKDRFSYDEKDMRCTDQYQFVDERGRRLKEKVEIKGFRYKIGEKGKDSKLHFTP |
| 4483 | FGLVAFISLFLEKKYSKILTDKLRLIPIQDQHIINEMLAVYRIRLNAQKLNISKDADLLALDIINELQRCPKDLF |
| | SLLSPSDQKKFRHESEANDEVLMVRHSDRFPFLVMKYIDDCQLFDNIRFQVSLGKYFYKFYDKNCIDSETR |
| | VRALSKNLNGFGRLSKIEAMREGCWEDSIRQYDDIHKNTVDEKPYVTDHHAKYVINGNRIAMRIIREEEKA |
| | YLPELNAEGVRNLAPTCWLSIYDLSSL |
| IMG_ | LXXXXXXXXXXXXXXXXXXXXXXXXXXXXXXXXXXXXXXXXTGLFRTRAQNGNSPFEKNENEVMFNI |
| 2061766007_3 | FCAHRIRLPKGRVESTASAHALGLDILNELQKCPSELFNTLSPEDKKQFQVKRKDDEIQPNPDDDLNLFRRN |
| SEQ ID NO: | GDRFPYLAMRYIDAMRETQDDQSKVLKDIVFQVSLGKYRFKFYNRASLDTQRNDRVRVQQKEINGFGPID |
| 4484 | KVEQKRKDKYSPIIRPISNDPKHLFXXXXXXXXXXXXXXXXXXXXXXXXXXXXXXXXXXXXXXXXXXE |
| | KTGDKGHNETINIEKLDNDKCFLPNVPISTENIAPRAWLSXXXXXXXXXXXXXXXXXXEAVIKDTYKRFV |
| | LLLKDIRSGDLKPQANKEQLQNXXXXXXXXXXXXXXXXXXXXXXXXSLRHRTENPRKS |
| OOXJ01.1 | MKQNTNNRQSKNKGRKNEGSFQELTPRFFDDVKTKAVWANYLNMARQNTYQTLCHITHVLGLAYNPED |
| SEQ ID NO: | KELEANLLQIPAVTLLLKKGNAEKKQKAMKLLDKHFPFMTPMLEQYVKLQQGKSTRGKETTPEDYHAILN |
| 4485 | MILPLINLLRNKYTHYKIEDPKLDASGKIADPGILNNCHILARLLNFCFDGARRIVKERFGTGENAPLKDKDF |
| | NFLTEEGTRYYKEDKKFIERKDFKYRIFDDTQEISNIGIFMLTCLLLEKKYASEFADQTDFFGKNLEPKRRPT |
| | ENEILIMREAVSVYRIRLPKDRMQSDRGESALGLDMLNELKKCPRELFDTLSPADQETFRVEANDNEDGKV |
| | LLLRSHDRFPTLALQYIDYKQLFAHIHFQVQLGNYRYKFYEKEWIDKSKEQTDKDGADERIRILQKELTGY |
| | GRLQEIESQRNERWGHIIRKIDAPRQDALDTQPYVTDHHASYLFNNNRIGLLWNTEKEHPLRNGVFMPSLE |
| | LPSWLDDYPAKAAELRGTAQKTDEKVAECRAPMCWLSTYELPAVIFLSLLTGSGQAAEELIKNTTAAYRR |
| | LFADIASGKLLPGGDLTPYGIELKLLPEKIQDYLTGKEVDMN |
| ULOJ01.1 | MKQNTNNRQSKNKGRKNEGSFQELTPRFFDDVKTKAVWANYLNMARQNTYQTLCHITHVLGLAYNPED |
| SEQ ID NO: | KELEANLLQIPAVTLLLKKGNAEKKQKAMKLLDKHFPFMTPMLEQYVKLQQEKSTRGKETTPEDYYAILN |
| 4486 | MILPLINLLRNKYTHYKIEDPKLDASGKIADPGILDNCHILARLLNFCFDGARRIVKERFGTGENAPLKDEDF |
| | DFLTEEGKRYYKEDKKFIERKDFKYRIFHDTQKISNIGIFMLTCLLLEKKYASEFADQTDFFGKNLEPQRRPT |
| | ENEILIMREAISVYRIRLPKDRMQSDRGESALGLDMLNELKKCPRELFDTLSPADQETFRVEANDNEDGKVL |
| | LLRSHDRFPTLTLQYIDYKQLFAHIRFQVQLGNYRYKFYEKEWIDKSKEQRDKDGADERIRILQKELTGYG |
| | RLQEIESQRNERWGHIIRKIDAPRQDALDTQPYVTDHHASYLFNNNRIGLLWNTEKEHPLRNGVFMPSLELP |
| | SWLDDYPAK |
| IMG_ | MSYKNQEEKYFFSVYLNLARLNAYLTLSHITKLLGKKPSPKEESLVTMPIIEALNGIDQLLLQKSQRLILKHF |
| 3300000230 | PFFKAIVEKEKSKATDENKLLYDVCKLFFHFLNEWRNFYTHYNHAPVNFQDDAEKENFFKYLDFIFDASLR |
| SEQ ID NO: | KGKERFTWDEKNLKRFRYKSGYDKVKKLPKENPDFQYQFHKNNDLTEKGFIYFVCMFLERKDSADLINAL |
| 4487 | AAVYNFQKTEESIFREIYSIYAIRIPHHRVESTDSMLTLGLDILNELKRFPKSLYEILRKSEKETFIENIKDEGQ |
| | NETNFKRFNERFPYFALNFIDELKLFKDYRFHVKLGKYYFQFYDKNTVDGEIRKRDLSVNLKTFGRINEVN |
| | DVRKKDWKDLIWDDNEGETPTPPKEYAKKYITNSFPRYILESNQIGLKKVPNVSLPELNDKKTRCLAPDCY |
| | LSVFELPALIFYGLLLNKNREAEAIMTFLPIELVIVKFISSVKKFFKHFHGGKSNYLFLKNKFPIRCPILQLI |
| UYCW01.1 | VFLSKLPNPGNYPSNSKESRIIRRSMGVCSVALPKERIHSETGDLSVALDMLNELKRCPRELFDTLSPGDQER |
| SEQ ID NO: | FRTISSDHNEVLQMRSKDRFAQLVLQYIDHNRLFENLRFHVNMGKLRYLFNPKKYCIDGQTRVRVLEHPLN |
| 4488 | GFGRLQEMEKERLQKDGTFADSGIKVRCFDEVRRDDADSNNYPYIVDTYTHYVLENDMVEMFFCPEGSG |
| | MKMPEVTSREGKWYVDKKVPHCRMRMSVLELPAMLFHLLLCGAKNTEVHIGKVCDNYCHLFSDMAQGN |
| | LTEENILSYGIKKEDIPQKVWDCVRGVHTGKDCRVFRKKEIRGRYEDVTRRLERLEADRKAVLGGENKIGK |
| | RGFVQIVPGRLAAYLATDICRLQPSLRKGAEYGTDRLTGMNFRLLQSSIATYNCGESDILYGRFRDMYSAV |
| | LD |
| ULPT01.1 | VFLSKLPNPGNYPSNSKESRIIRRSMGVCSVALPKERIHSETGDLSVALDMLNELKRCPRELFDTLSPGDQER |
| SEQ ID NO: | FRTISSDHNEVLQMRSKDRFAQLVLQYIDHNRLFENLRFHVNMGKLRYLFNPKKYCIDGQTRVRVLEHPLN |
| 4489 | GFGRLQEMEKERLQKDGTFADSGIKVRCFDEVRRDDADSNNYPYIVDTYTHYVLENDMVEMFFCPEGSG |
| | MKMPEVTSREGKWYVDKKVPHCRMRMSVLELPAMLFHLLLCGAKNTEVHIGKVCDNYCHLFSDMAQGN |
| | LTEENILSYGIKKEDIPQKVWDCVRGVHTGKDCRVFRKKEIRGRYEDVTRRLERLEADRKAVLGGENKIGK |
| | RGFVQIVPGRLAAYLATDICRLQPSLRKGAEYGTDRLTGMNFRLLQSSIATYNCGESDILYGRFRDMYSAV |
| | LD |
| OUQN01.1 | LSVFGKKYVNVFLQKLPIYGTYKKQSLEANIIRQTFGIHTAKLPKERIVSEKSDFSIGMDMLNELKRCPKALF |
| SEQ ID NO: | STLSYADQNAFRIVSSDMNDVLQVRHTDRFAQLSLEYIDRRELFSDIRFHLNMGKLRYLKTADKHCIDGISR |
| 4490 | VRVLEDKINAFGRIHEFEARRKELGFVECYEQGGRAISTNTNIEIRDFEHVKRDDSNPDSYPYIIDTYTHYILE |
| | NNKIGMHIGDYWPDLIKLDEHKWTVYNENPTCFMSSLELPAMMFHMMLCGSDATESLIKAEVDKYKKLF |
| | GAMANGTLTKENISGFGIAEENIPQKVIDCVNGKTSGKGLDKQIKKEIDEMLADTNLRIERLKSDKRSVAST |
| | QNKMGKRGFRSIQPGKLADWLAADIVKHQPSLLKGVDYGTDRITGMNYRVMQSTIATFNATTPEHSLEEL |
| | KKVFSAAQFIQCEKKEHPFLYKALDRNPQNTIDLYEFYLSARQSYYKSMRRNIENGENVKLPYLNTDRNK |
| | WMRRGSVYYSTMGEIYLKDMPIELPRQMFDKKIKEALAKLPSMKDVDMQHCNVTFLIAEYLKKELKDDS |
| | QPFYQWNRNYRFTDMMICEENRSTRALSTHFIPVALREEIWEKRSELKAAYKEWALPRLSKNRDTERLSPA |
| | QKSELLDARIAKCRNEYQKNEKIIRRYKVQDALMFMMVKDMFGKGVFTAESKEFALSAITPDAKRGILSEV |
| | IPIDFKFSIDGKTYTIHSNGMKIKNYGDFYKLINDKRMKSILKIITHNVIDKDLLEKEFSSYDDKRPEAIEIVFE |
| | FEKAAYSKYPELEELVLSENHFDFGTLLRELQAKKVLSQNDGHYLSQIRNAFSHNSYPRNLRIPSNIPEIAQE |
| | MINIFRITTPLKTKK |
| OQWI01.1 | LSVFGKKYVNVFLQKLPIYGTYKKQSMEANIIRQTFGIHTAKLPKERIVSEKSDFSIGMDMLNELKRCPKAL |
| SEQ ID NO: | FSTLSYADQNAFRIVSSDMNDVLQVRHTDRFAQLSLEYIDRSELFSDIRFHLNMGKLRYLKTADKHCIDGIS |
| 4491 | RVRVLEDKINAFGRIHEFEARRKEQGFVEGYEQGGRAISTNTNIEIRDFEHVKRDDSNPDSYPYIIDTYTHYI |
| | LENNKIGMHIGDYWPDLIKLDEHKWTVYNENPTCFMSSLELPAMMFHMMLCGSDATESLIKAAVDKYKK |
| | LFGAMANGTLTKENISGFGIAEENIPQKVIDCVNGKTSGKGLDKQIKKEIDEMLADTNLRIERLKSDKRSVA |
| | STQNKMGKRGFRSIQPGKLADWLAADIVKHQPSLLKGVDYGTDRITGMNYRVMQSTIATFNATTPEHSLE |
| | ELKKVFSAAQLIQCEKKEHPFLYKALNRNPQNTIELYEFYLSAKQSYYKSMRRNIENGENVKLPYLNTDRN |
| | KWMRRGSVYYSTMGEIYLKDMPIELPRQMFDKKIKEALAKLPSMKDVDMQHSNVTFLIAEYLKKELKDD |
| | FQPFYQWNRNYRFTDMMICEENRSTRALSTHFIPVALREEIWEKRSELKAAYKEWALPRLSKNRDTERLSP |
| | AQKSELLDARIAKCRNEYQKNEKIIRRYKVQDALMFMMVKDMFGKGVFTAESKEFALSAITPDAKRGILSE |
| | VIPIDFKFSIDGKTYTIHSNGMKIKNYGDFYKLINDKRMKSILKIITHNVIDKDLLEKEFSSYDDKRPEAIEIVF |
| | EFEKAAYSKYPELEELVLSENHFDFGTLLRELQAKKVLSQNDGHYLSQIRNAFSHNSYPRNLRITSNIPEIAQ |
| | EMINIFRITTPLKTKK |
| GCA_ | MKIPQIIEDNKHLFGTYSTMALANIRNILDHIATLACIENDFNADSDDFWHHPCMEIINPQNLCNDVTKADF |
| 900543255.1_ | VTEKLKSHFPFVVIMAEAKRQKDIAWAKNQAKKAFENRDFQKQQEFNKKQKSLLSITNADIYRVLNNLFR |
| UMGS549_ | VLTSYRHYTSHYLINYIYFNEGSNLLKYHEQPLSYNINDYFTIALRDTAQKYSYSPEALSFIQSSRYKIENRR |
| genomic | KILDTDFFLSIQHRNGDSSPKNLHISGVGVALLICLFLEKKYVNVFLQKLPIYGTYKKQSMEANIIRQTFGIHT |
| SEQ ID NO: | AKLPKERIVSEKSDFSIGMDMLNELKRCPKALFSTLSYADQNAFRIVSSDLNDVLQVRHTDRFAQLSLEYID |
| 4492 | RRELFSDIRFHLNMGKLRYLKTADKHCIDGISRVRVLEDKINAFGRIHEFEARRKELGFVECYEQGGRAIST |
| | NTNIEIRDFEHVKRDDSNPDSYPYIIDTYTHYILENNKIGMHIGDYWPDLIKLDEHKWTVYNENPTCFMSSL |
| | ELPAMMFHMMLCGSDATESLIKAEVDKYKKLFGAMANGTLTKENISGFGIAEENIPQKVIDCVNGKTSGK |
| | GLDKQIKKEIDEMLADTNLRIERLKSDKRSVASTQNKMGKRGFRSIQPGKLADWLAADIVKHQPSLLKGV |
| | DYGTDRITGMNYRVMQSTIATFNATTPEHSLEELKKVFSAAQLIQCEKKEHPFLYKALDRNPQNTIDLYEFY |
| | LSARQSYYKSMRRNIENGENVKLPYLNTDRNKWMRRGSVYYSTMGEIYLKDMPIELPRQMFDKKIKEALA |
| | KLPSMKDVDMQHSNVTFLIAEYLKKELKDDFQPFYQWNRNYRFTDMMICEKTALQEH |
| OJAW01.1 | MKIPQIIEDNKHLFGTYSTMALANIRNILDHIATLACIENDFNADSDDFWHHPCMEIINPQNLCNDVTKADF |
| SEQ ID NO: | VTEKLKSHFPFVVIMAEAKRQKDIAWAKNQAKKAFENRDFQKQQEFNKKQKSLLSITNADIYRVLNNLFR |
| 4493 | VLTSYRHYTSHYLINYIYFNEGSNLLKYHEQPLSYNINDYFTIALRDTAQKYSYSPEALSFIQSSRYKIENRR |
| | KILDTDFFLSIQHRNGDSSPKNLHISGVGVALLICLFLEKKYVNVFLQKLPIYGTYKKQSMEANIIRQTFGIHT |
| | AKLPKERIVSEKSDFSIGMDMLNELKRCPKALFSTLSYADQNAFRIVSSDLNDVLQVRHTDRFAQLSLEYID |
| | RRELFSDIRFHLNMGKLRYLKTADKHCIDGISRVRVLEDKINAFGRIHEFEARRKELGFVECYEQGGRAIST |
| | NTNIEIRDFEHVKRDDSNPDSYPYIIDTYTHYILENNKIGMHIGDYWPDLIKLDEHKWTVYNENPTCFMSSL |
| | ELPAMMFHMMLCGSDATESLIKAEVDKYKKLFGAMANGTLTKENISGFGIAEENIPQKVTDCVNGKTSGK |
| | GLDKQIKKEIDEMLADTNLRIERLKSDKRSVASTQNKMGKRGFRSIQPGKLADWLAADIVKHQPSLLKGV |
| | DYGTDRITGMNYRVMQSTIATFNATTPEHSLEELKKVFSAAQLIQCEKKEHPFLYKALDRNPQNTIDLYEFY |
| | LSARQSYYKSMRRNIENGENVKLPYLNTDRNKWMRRGSVYYSTMGEIYLKDMPIELPRQMFDKKIKEALA |
| | KLPSMKDVDMQHSNVTFLIAEYLKKELKDDFQPFYQWNRNYRFTDMMICEKTALQEH |
| OVJZ01.1 | MRIPSLIENNKKYYAIHSEMALLNAQAVLDHIQKMAGIEACAYNEKEKKPSDEDLWVHPVMIFLDKAKTS |
| SEQ ID NO: | EVKAEKVQYVIERLCSYFPFMNIMAQFQREYDNEHNKTNRLEVNANDMYDALNKIFRVLKKYRDYSAHY |
| 4494 | KFEDNCFIDGCAFLRYSEQPLASMVRKYYDVALRNIKEKYNYKTEELAFIQNKRYKITKGIDGRKKTVGNP |
| | NFFLTLTSNNGDTTNKWHLSGVGVALLISLFLDKQYVNLFWTRLPIFSDNKLKEDERRVIIRSMGINSVKLP |
| | KDRIHMDKDDMSVAMDMLNELKRCPDELFDILPAEKQAHFRIISSDHNEVLMKRSTDRFTSMLLQYIDYG |
| | KKFKQIRFHVNMGKLRYLLNAEKNCIDGNIRTRVTEHPLNGYGRIDEIEELRKNEDMTYADTGIRIKDFESM |
| | TRDDSDTANYPYVVDTYTHYLLENNKVEFSFCGNSSLPEVSERNGKWYVSKDVPACRMSILELPAMAFHM |
| | LLLGSEKTEARIKSVYD |
| IMG_ | MAQTRWKQSKTPAIKPVMAAYLNMARHNMYRVMLHISRQMQIIENKEEAEIAAFSVWQKLSSGTPTEQM |
| 3300028914 | KMIKLLQRHFPVLKPVFDVEKKKNVENAAISASPKEIKRIFTTILTALNRLRNEYSHYSPVPRKTEGEEKMIA |
| SEQ ID NO: | YLYRCMDGSAREVRNRFSLTVKDPKGAKETAKVLEVNKAVFDIFQDAFRKEKVKALDKSGKVTKDNKGR |
| 4495 | TQFEFRDKEDYYYALKDANAALSDMGIVFFTCLFLEKRYAAMFLDAIKPWPQDFNEIERKAVLEVFTVYHI |
| | HLPKEKYDSTRPEYALGLDMLNELQKCPKELFDILSAKSRDALSVDIKADRPDVVTDDGVTVKDGKVQMR |
| | RVRDRFAPLALQYLDSQKAFNDIRFMVRLGHYRFKFYKKQCVADNAPDTLRVLQKEINGFGRLDEMEAA |
| | RKKNYTPLFKATCVKTNDKGIEVHELVPDAPDSAPYITDTKAHYLIDNNRVGLRIDNPSFLPSLRGEKGAPI |
| | QSAGDISLLSPQAWLSTYELPGLVFYQYLYDTYDGHGKHLPSAEEIIKSYILAYKRLFVDLGEGSFDGWDEY |
| | AYAPLTLGDLPQKIKGFILHPSATIDPRFQDKANNRIDDMIKRTEAEIAGFDTKMKKLSDKSNKLGKKKYV |
| | DIRPGSIASRLVRDILFFTPVSEEKAKITSANFNSLQSALALSELGTNRIKDILRGLNHPFVMKAFEKYRVEDF |
| | HLFDFWKVYLNKRLDYLKGLDREKLEEVPFLHSSRTRWQKRDEKYIKLLAGRYEQFELPRSLFTAPTRVLL |
| | DEVALHFESGSDRDLSMGNLINLFFSKVLSDNNQPFYRWERHYDVFDKLAGVKSGISLVHQFFKPEQLAKK |
| | MRERKTLKPSLYMCEKAVNSVNQNLK |
| GCA_ | MFLDAIKPWPQEFNDTEKKAVLEVLSVFHIRPPKEKYDSQRPDYALGLDMLNELQMCPSELFEVLSDKSRD |
| 002438905.1_ | MLSVDIHAQGEDVVQDDGVTGRDGKVQMKRIRDRFAPLALQYIDRQEVFDNIRFMVRLGNYRFKFYKKQ |
| ASM243890v1_ | CLADNGPDTLRILQKEINGFGRIQEVEIERKRKYSALFKKTRTTTDESEAKTKIQELVADTPDSKPYMTDTK |
| genomic | VHYLFSNNRVGLRLFNDSSKLDIPEVTKQGLPLTSASEVKLLLPDAWLSIYELPGLIFYQHLYKEYGAKGNY |
| SEQ ID NO: | PSAEDILKSYIDAYRRLFSDIEDGTFLGWDDTKYKPLSQDVLPIKIKKYIQNGNGVQSAYFHKKARERIKEM |
| 4496 | CEQTQAELNGFKSKISKMTSKDNKFKKGHYVDLRPGSISMRLCRDILFFMEIPEEKSVITSANFNSLQSALA |
| | MSATTNDKVDEMLSPLKHPFLQEALKKYHNLSNGKYFKVFDFYKIYLEKRANYLELLKKISSDQLIKLPFL |
| | HYSRIRWRDRSNSSIKELAGRYEQFELPRSLFTQYAKKILVENCALSLEAETAERKLGMSNLVNTYFQTVM |
| | NDTTQKFYRWPRHYRAFDLLGGKTIRNQVVHEFMTPIQLQKMMRDRKSLKPNGLILDKAKNAVKQEKGK |
| | KKITDKDLANQILRKVYKEYDENERTIRRYAVQDMLMFLMAKDILLGIDGIEKESLDKFKLKDILPNNKETI |
| | LELMVPFNVSLIVNGINVTIHQEENIKIKRFGEFYRYNSDTRLKSLIPYLVKNLGTCAGVSIEIDRDKLETELS |
| | QYDLNRIEVMKQVQSLEQSIIAGAGGKNNIDKTLRENFNNLITIQGNIPYENQGRVLINVRNAFCHNEYAKD |
| | IDIPANTPLPQVADAIVKLFKTEKRRNKRKDN |
| UYAX01.1 | MNTHQEELQSWITRKRLPDTEMKKYWAAYMNLARLNFFKTLMFISNSIGDLKPAKDNNGKGNTEVNMHN |
| SEQ ID NO: | MGILTALLGPEDEEKARLLIFKHFPFLRTFCIEKELSLSKQRTILIDMACIIGRYRNMYSHSIFISDDNEKVLES |
| 4497 | EKRCSEYLQSILTVSTRIIKERYRSNKNDAQRGMIDDKSLKFISENKVKFVYDENGKRITAPNKKYYLSTIDK |
| | DNTHLSYFGKLMLTCILLEKKYATDFLTQCHFLDAFNDSEVAPKLSERRLMLEVMTALRIRLAEKKLSNEK |
| | SEVQISLDILNELKKCPFELYELLGSEDKRLFTIVADTGETILLRRHEDRFPQLALSWIDSSKAFDHLRFQVNA |
| | GKLRYLFRDNKHCIDGQTRMRVLEEPLNGYRRLMEFEEERIQKQSGEIRSLWPGLDILNKDETPRNDASVL |
| | PYISDYRVRYLFDGDNIGISIGDFTPSITKTDETKYRVTGKTADCCLSKYELPGLLFYHLLTLRHGDNKRSTK |
| | HA |
| OOVA01.1 | MNTHQEELQSWITRKRLPDTEMKKYWAAYMNLARLNFFKTLMFISNSIGDLKPAKDNNGKGNTEVNMHN |
| SEQ ID NO: | MGILTALLGPEDEEKARLLIFKHFPFLRTFCIEKELSLSKQRTILIDMACIIGRYRNMYSHSIFISDDNEKVLES |
| 4498 | EKRCSEYLQSILTVSTRIIKERYRSNKNDAQRGMIDDKSLKFISENKVKFVYDENGKRITAPNKKYYLSTIDK |
| | DNTHLSYFGKLMLTCILLEKKYATDFLTQCHFLDAFNDSEVAPKLSERRLMLEVMTALRIRLAEKKLSNEK |
| | SEVQISLDILNELKKCPFELYELLGSEDKRLFTIVADTGETILLRRHEDRFPQLALSWIDSSKAFDHLRFQVNA |
| | GKLRYLFRDNKHCIDGQTRMRVLEEPLNGYRRLMEFEEERIQKQSGEIRSLWPGLDILNKDETPRNDASVL |
| | PYISDYRVRYLFDGDNIGISIGDFTPSITKTDETKYRVTGKTADCCLSKYELPGLLFYHLLTLRHGDNK |
| ULIX01.1 | MNTHQEELQSWITRKRLPDTEMKKYWAAYMNLARLNFFKTLMFISNSIGDLKPAKDNNGKGNTEVNMHN |
| SEQ ID NO: | MGILTALLGPEDEEKARLLIFKHFPFLRTFCIEKELSLSKQRTILIDMACIIGRYRNMYSHSIFISDDNEKVLES |
| 4499 | EKRCSEYLQSILTVSTRIIKERYRSNKNDAQRGMIDDKSLKFISENKVKFVYDENGKRITAPNKKYYLSTIDK |
| | DNTHLSYFGKLMLTCILLEKKYATDFLTQCHFLDAFNDSEVAPKLSERRLMLEVMTALRIRLAEKKLSNEK |
| | SEVQISLDILNELKKCPFELYELLGSEDKRLFTIVADTGETILLRRHEDRFPQLALSWIDSSKAFDHLRFQVNA |
| | GKLRYLFRDNKHCIDGQTRMRVLEEPLNGYRRLMEFEEERIQKQSGEIRSLWPGLDILNKDETPRNDASVL |
| | PYISDYRVRYLFDGDNIGISIGDFTPSITKTDETKYRVTGKTADCCLSKYELPGLLFYHLLTLR |
| OZPT01.1 | LILSFIIFNFIVIINQHTLNTYTRKYMKDKSFSTISSAINETITTDNIKYPEQLNLILSKRLRPKNELQPLWAAYF |
| SEQ ID NO: | NMARYNMYTTLVHIATATGLSDEDNMENRMDKMRILNEPVEPEIEHRLRKLLCRHFPFAVWMICSPIRKK |
| 4500 | DSKEDSADEDYRVISVKELRDCLKTVSYTLNYFRNYYSHTRHVETRSEDIIAASRNSEKQTGIFLNKVCTVA |
| | TRRVKSRFSDKSNKGQAGMIDDQSMKFITEGKVKFRNNNGIKETIYNPDHFLYPLFLNRSSALRDGTNPERL |
| | STVGKIQLICLLLDKKYITEFLDQSGFLSAFNNDAPAPKLSERRLILEVLSDLRIRLPQRKIDATCNDIQVALD |
| | MLNELKKCPKELFELLEAKDKATFSILSSTGEHILLRRSSDRFTQLALQWFDVNKAFSRIRFHMNAGIFRYLF |
| | NDSKTCIDGKTRLRVLQEPLNCFGRIQEVEDSRESNRDGNDGPWRGFEIKGFDEAARNDVNCLPY |
| IMG_ | MGYLPSYVLAAYYNDARLNIFACLNDVRQKLGKQALDNDDQIVSAIKELGLTKATPEDQARIIQYLHASFG |
| 3300005479 | FLGVFMDVTKAKNKQTNPEQATPLPRYYEERLIWLFSLVNDLRNTFVHPTDGECEIPRLVHRRLYFLLSRV |
| SEQ ID NO: | YDASFHVLKTRFSYSTEAMRPFMRCDQKGKPKRANQFLFALASDPLNLTDQTKTPQSQVFHAFGQVLFCS |
| 4501 | LFLEKSQSAELISHFWEFVPQKLQAAWSTEQRKLIRELITIYRLRLPLQRLQSTDSTVAITLDSLAELSRCPLP |
| | LFETLSLEDQARFRFEAETTSDAEEAGSSVLFARSRDERFPSLMMRFLDFDPTNRLRFAVDLGQLHYHVRL |
| | KSAEHFTDQRARIRSLGQKIVAYGCLQAFEQAEKPADWQILENNYTQMRAEAEGLIQEAASGMSTLRPYLI |
| | PAYPHYHYFPERIGFRVDQAKTKQTASYPDLQAVTAEQAVRLEPPSAQDMQPQFWMSHEQLLQLSFYHFL |
| | WKQQGADAKQPSLDQLLLRYESGMKRLFKALSAGDGLECKTPAELQAWLDELFNTRQQFAVPVSSLPKV |
| | LVQHLLTKKQKPITRAMVEQRIQHLLAETDYRLEQLKTILASEKKRGQKGFKLLKCGPIGDFLAEDLLRFQ |
| | AVDSSKSDGGKLNSQQYQILQKTLAYYGAHLEEPPKITDLLADFGLLSGAWAHPFLADLGLTQRPDQYQG |
| | LLSFYAAYLKARRRFLKRFAAIPKQWSLQALPAWLGLKPKATLANWQAELWDGEQLRQPLPVPDQFLYRP |
| | ILNLVAAALQLAPQALEQEGSVSYEQGGAKVWIPPSVTWLLKRYLAAQGQEMQAMYTYPRRHHLLDTWL |
| | DQRSKQFAEKHKHYLPETERQQYTVAIRQWC |
| AATN01.1 | MGYLPSYVLAAYYNDARLNIFACLNDVRQKLGKQALDNDDQIVSAIKELGLTKATPEDQARIIQYLHASFG |
| SEQ ID NO: | FLGVFMDVTKAKNKQTNPEQATPLPRYYEERLIWLFSLVNDLRNTFVHPTDGECEIPRLVHRRLYFLLSRV |
| 4502 | YDASFHVLKTRFSYSTEAMRPFMRCDQKGKPKRANQFLFALASDPLNLTDQTKTPQSQVFHAFGQVLFCS |
| | LFLEKSQSAELISHFWEFVPQKLQAAWSTEQRKLIRELITIYRLRLPLQRLQSTDSTVAITLDSLAELSRCPLP |
| | LFETLSLEDQARFRFEAETTSDAEEAGSSVLFARSRDERFPSLMMRFLDFDPTNRLRFAVDLGQLHYHVRL |
| | KSAEHFTDQRARIRSLGQKIVAYGCLQAFEQAEKPADWQILENNYTQMRAEAEGLIQEAASGMSTLRPYLI |
| | PAYPHYHYFPERIGFRVDQAKTKQTASYPDLQAVTAEQAVRLEPPSAQDMQPQFWMSHEQLLQLSFYHFL |
| | WKQQGADAKQPSLDQLLLRYESGMKRLFKALSAGDGLECKTPAELQAWLDELFNTRQQFAVPVSSLPKV |
| | LVQHLLTKKQKPITRAMVEQRIQHLLAETDYRLEQLKTILASEKKRGQKGFKLLKCGPIGDFLAEDLLRFQ |
| | AVDSSKSDGGKLNSQQYQILQKTLAYYGAHLEEPPKITDLLADFGLLSGAWAHPFLADLGLTQRPDQYQG |
| | LLSFYAAYLKARRRFLKRFAAIPKQWSLQALPAWLGLKPKATLANWQAELWDGEQLRQPLPVPDQFLYRP |
| | ILNLVAAALQLAPQALEQEGSVSYEQGGAKVWIPPSVTWLLKRYLAAQGQEMQAMYTYPRRHHLLDTWL |
| | DQRSKQFAEKHKHYLPETERQQYTVAIRQ |
| IMG_ | MRLDQAVLAAFYNDARLNILSCLNDIREKQGLSFIGDDAQIVSAFNDLSHILTQGTPEEASALIDRLRYRFPF |
| 3300021975 | IDTRTDASSRHKDFTPVPDNFQHIFQRIFKLINSLRNTLVHPVNSPLLLDMDQHKNLFFMLNDIYDDARRLL |
| SEQ ID NO: | KTRFDWSTRDLMPLLRCDHKGKPKAVNKFSFALCSDPKSRSNSPVIGYNRVLYDFGHVLLCSLFLDKSQSA |
| 4503 | DLIHHFWQSGHGKFWQNQKHREMIKELISAYHIRLPLQRLKADSLTTLTIDALSELSRCPQPLLKTLKKEDK |
| | DKFREALGTLDNIDISDDAGNGAGNAAANSARKQSQAEQQASYLLARSHEDRFVPLMMRFLEHDPANKLR |
| | FAIDLGQFYYHVRLKSGDFFTDNKPRVRRLGQKLICYGHLNNLNKSDFWQQLEDNFALSSQEAKQAETLA |
| | SAEPLQLKPYLVKTIPHYHFDNNKIGFRLAQSTGKSTDKRANKSANQKTDYPKYPEDKGISVEDIRQPIQLD |
| | KIPAEQMQAEFWISPAQMLHIGFYHYLYQHQQNATSGKATQSKSIEVLLNTYKAGTLRLFKALKKQMPEL |
| | AGEAFSAERHQAVQDYINGFYAAKGNNSDNSDYHISMANLPKVLVNALLGAQQQQVIPKQQIIDRANKLL |
| | QSTEQRQRQLERQLNAFKKRGHKDFRPIKCGNIGDFLTDDIIRFQAVDPSQNDGGKLNSQQYQILQKTLAY |
| | YGKYIDEPPQIIDLFQDFGLIEGDFKHPFLDKLGLQKNPHKYKGLLDFYKDYEKLILKSILIELAVMTIINNNR |
| | YKLHTGCA |
| IMG_ | MRLDQAVLAAFYNDARLNILNCLNDIREKQGLSIIGAGDHADATQIVSAFNDLSYILTHGTPEEASALIDRL |
| 3300021792 | RYRFPFIDTCTDANSRHKDFTPVPDNFQHIFERIFKLINALRNTLVHPVNTPLLLDMDQHKDLFFMLNDIYD |
| SEQ ID NO: | DARRLLKTRFDWSNDELKPLLRCDHKGKPKAVNKFSFALCSAPNSASKSRSNHTAIGYNEVLYDFGHVLL |
| 4504 | CSLFLDKSQSADLITYFWQSGHDKFWRNPKHREMIKELISVYHIRLPLQRLKADSLKTLTIDALSELSRCPRP |
| | LLKTLKKEDKDKFREALDPLDNIDPENVAGNGAIDSSAKSQNQVEQQASYLLARSHEDRFIPLMMRFLEHD |
| | PANKLRFAIDLGHFYYHVRLKSGNFFTDNKPRVRRLGQKLITYGHLNSLNKSDFWQQLEDNFALSNQEAE |
| | QAKKIANAKPLQLKPYLIKTIPHYHYDHNKIGIRLAQSTDKSTDKSTDKSTDKNANQKTDYPKYPEDKGIN |
| | V |
| IMG_ | MESIIGLGLSFNPYKTADKHYFGSFLNLADNNLKAVFAEFKERISDKSKDEDISNLIEKHFIDNMSIVDYEKN |
| 3300031208 | ISILNGYLPIIDFLDDELENNLNTRVKNFKKSFIILAEALETLRNYYTHFYHDPITFGDNKEPLLELLDEVLLKT |
| SEQ ID NO: | ILDVKKKYLKTDKTKEILKDSLREEMDLLVIRKTDELREKKKTNPKFKFSTDPTQIRNSIFNDAFQGLLYED |
| 4505 | KENNGKTQVSYRAKTKLNPKDIHKQEERDFEIPLSTSGIVFLMSLFLSKKEIEDFKSNIKGFKGKVVKDENH |
| | NSLKYMATHRVYSILAFKGLKYRIKTDTFSKVTLMMQMIDELSKVPDCVYQNLSETKQKDFIEDWNEYLK |
| | DNEENTENLENSRVVHPLIRKRYEDKFNYFAIRFLDEFANFKTLKFQVFMGYYIHDQRTKTIGTTNITTERT |
| | VKEKINVFGKLSKMDNLKKHFFSQLSDEENTDWEFFPNPSYNFLTQADNSPANNIPIYLELKNQQIIKEKDDI |
| | KAEVNNSQNRNPNKPSKRDLLNKISNTNEDFYQGDPTAILSLNEIPALLHLFLVQPDNKTGQQIENIIRIKIEK |
| | QFNSIKNPSKDDKGVPKSLFADTNVRVNAIKLKKDLGEELDMLNKKQIVFKENQKASSNYDELLKKHQFTP |
| | KNKRPALRKYVFYNSEKGEEATWLANDIKRFMPKGFKTKWKGYQHSELQRKLAFYDRHTKQDIKELLSG |
| | CEFDHFLLDINACFKEDD |
| IMG_ | MESIIGLGLSFNPYKTADKHYFGSFLNLADNNLKAVFAEFKERISDKSKDEDISNLIEKHFIDNMSIVDYEKN |
| 3300028348 | ISILNGYLPIIDFLDDELENNLNTRVKNFKKSFIILAEALETLRNYYTHFYHDPITFGDNKEPLLELLDEVLLKT |
| SEQ ID NO: | ILDVKKKYLKTDKTKEILKDSLREEMDLLVIRKTDELREKKKTNPKFKFSTDPTQIRNSIFNDAFQGLLYED |
| 4506 | KENNGKTQVSYRAKTKLNPKDIHKQEERDFEIPLSTSGIVFLMSLFLSKKEIEDFKSNIKGFKGKVVKDENH |
| | NSLKYMATHRVYSILAFKGLKYRIKTDTFSKVTLMMQMIDELSKVPDCVYQNLSETKQKDFIEDWNEYLK |
| | DNEENTENLENSRVVHPLIRKRYEDKFNYFAIRFLDEFANFKTLKFQVFMGYYIHDQRTKTIGTTNITTERT |
| | VKEKINVFGKLSKMDNLKKHFFSQLSDEENTDWEFFPNPSYNFLTQADNSPANNIPIYLELKNQQIIKEKDDI |
| | KAEVNNSQNRNPNKPSKRDLLNKISNTNEDFYQGDPTAILSLNEIPALLHLFLVQPDNKTGQQIENIIRIKIEK |
| | QFIEVSS |
| IMG_ | MESIIGLGLSFNPYKTADKHYFGSFLNLADNNLKAVFAEFKERISDKSKDEDISNLIEKHFIDNMSIVDYEKN |
| 3300028412 | ISILNGYLPIIDFLDDELENNLNTRVKNFKKSFIILAEALETLRNYYTHFYHDPITFGDNKEPLLELLDEVLLKT |
| SEQ ID NO: | ILDVKKKYLKTDKTKEILKDSLREEMDLLVIRKTDELREKKKTNPKFKFSTDPTQIRNSIFNDAFQGLLYED |
| 4507 | KENNGKTQVSYRAKTKLNPKDIHKQEERDFEIPLSTSGIVFLMSLFLSKKEIEDFKSNIKGFKGKVVKDENH |
| | NSLKYMATHRVYSILAFKGLKYRIKTDTFSKVTLMMQMIDELSKVPDCVYQNLSETKQKDFIEDWNEYLK |
| | DNEENTENLENSRVVHPLIRKRYEDKFNYFAIRFLDEFANFKTLKFQVFMGYYIHDQRTKTIGTTNITTERT |
| | VKEKINVFGKLSKMDNLKKHFFSQLSDEENTDWEFFPNPSYNFLTQADNSPANNIPIYLELKNQQIIKEKDDI |
| | KAEVNNSQNRNPNKPSKRDLLNKISNTNEDFYQGDPTAILSLNEIPALLHLFLVQPDNKTGQQIENIIRIKIEK |
| | QFIEVSS |
| IMG_ | MESIIGLGLSFNPYKTADKHYFGSFLNLADNNLKAVFAEFKERISDKSKDEDISNLIEKHFIDNMSIVDYEKN |
| 3300012128 | ISILNGYLPIIDFLDDELENNLNTRVKNFKKSFIILAEALETLRNYYTHFYHDPITFGDNKEPLLELLDEVLLKT |
| SEQ ID NO: | ILDVKKKYLKTDKTKEILKDSLREEMDLLVIRKTDELREKKKTNPKFKFSTDPTQIRNSIFNDAFQGLLYED |
| 4508 | KENNGKTQVSYRAKTKLNPKDIHKQEERDFEIPLSTSGIVFLMSLFLSKKEIEDFKSNIKGFKGKVVKDENH |
| | NSLKYMATHRVYSILAFKGLKYRIKTDTFSKVTLMMQMIDELSKVPDCVYQNLSETKQKDFIEDWNEYLK |
| | DNEENTENLENSRVVHPLIRKRYEDKFNYFAIRFLDEFANFKTLKFQVFMGYYIHDQRTKTIGTTNITTERT |
| | VKEKINVFGKLSKMDNLKKHFFSQLSDEENTDWEFFPNPSYNFLTQADNSPANNIPIYLELKNQQIIKEKDDI |
| | KAEVNNSQNRNPNKPSKRDLLNKISNTNEDFYQGDPTAILSLNEIPALLHLFLVQPDNKTGQQIENIIRIKIEK |
| | QFIEVSS |
| IMG_ | LKAVFAEFKERISDKSKDEDISNLIEKHFIDNMSIVDYEKNISILNGYLPIIDFLDDELENNLNTRVKNFKKSFI |
| 3300023981 | ILAEALETLRNYYTHFYHDPITFGDNKEPLLELLDEVLLKTILDVKKKYLKTDKTKEILKDSLREEMDLLVIR |
| SEQ ID NO: | KTDELREKKKTNPKFKFSTDPTQIRNSIFNDAFQGLLYEDKENNGKTQVSYRAKTKLNPKDIHKQEERDFEI |
| 4509 | PLSTSGIVFLMSLFLSKKEIEDFKSNIKGFKGKVVKDENHNSLKYMATHRVYSILAFKGLKYRIKTDTFSKV |
| | TLMMQMIDELSKVPDCVYQNLSETKQKDFIEDWNEYLKDNEENTENLENSRVVHPLIRKRYEDKFNYFAI |
| | RFLDEFANFKTLKFQVFMGYYIHDQRTKTIGTTNITTERTVKEKINVFGKLSKMDNLKKHFFSQLSDEENTD |
| | WEFFPNPSYNFLTQADNSPANNIPIYLELKNQQIIKEKDDIKAEVNNSQNRNPNKPSKRDLLNKISNTNEDFY |
| | QGDPTAILSLNEIPALLHLFLVQPDNKTGQQIENIIRIKIEKQFNSIKNPSKDDKGVPKSLFADTNVRVNAIKL |
| | KKDLGEELDMLNKKQIVFKENQKASSNYDELLKKHQFTPKNKRPALRKYVFYNSEKGEEATWLANDIKRF |
| | MPKGFKTKWKGYQHSELQRKLAFYDRHTKQDIKELLSGCEFDHFLLDINACFKEDDFEDFFSKYLKNRIET |
| | LNIILKQLHDFKNEPTPLKGVFKNCLKFLKQKNYVTENPEIIKKRILAKPAFLPRGIFDERPTMKKGKKSFDR |
| IMG_ | MSLFLSKKEIEDFKSNIKGFKGKWKDENHNSLKYMATHRVYSILAFKGLKYRIKTDTFSKVTLMMQMIDE |
| 3300024002 | LSKVPDCVYQNLSETKQKDFIEDWNEYLKDNEENTENLENSRVVHPLIRKRYEDKFNYFAIRFLDEFANFK |
| SEQ ID NO: | TLKFQVFMGYYIHDQRTKTIGTTNITTERTVKEKINVFGKLSKMDNLKKHFFSQLSDEENTDWEFFPNPSYN |
| 4510 | FLTQADNSPANNIPIYLELKNQQIIKEKDDIKAEVNNSQNRNPNKPSKRDLLNKISNTNEDFYQGDPTAILSL |
| | NEIPALLHLFLVQPDNKTGQQIENIIRIKIEKQFNSIKNPSKDDKGVPKSLFADTNVRVNAIKLKKDLGEELD |
| | MLNKKQIVFKENQKASSNYDELLKKHQFTPKNKRPALRKYVFYNSEKGEEATWLANDIKRFMPKGFKTK |
| | WKGYQHSELQRKLAFYDRHTKQDIKELLSGCEFDHFLLDINACFKEDDFEDFFSKYLKNRIETLNIILKQLH |
| | DFKNEPTPLKGVFKNCLKFLKQKNYVTENPEIIKKRILAKPAFLPRGIFDERPTMKKGKNPLIDRDEFAKWF |
| | VEYLENKDYQKFYNSEEYRIRDADFKKNAVIKKQKLKDFYTLQMVNYLLKEVFGKDEMNLQLSELFQTR |
| | QERLKLQGIAKKQMNKETGDSSENTRNQTYIWNKDVPVSFFNGKVTIDKVKLKNIGKYKRYERDERVKTF |
| | IGYEVDEKWMMYLPHNWKDRYSVKPINVTDLQIQEYEEIRSHELLKEIQNLEQYIYDHTTDKNTLLQDGNP |
| | NFKMYVLNGLLTGIKQVNIADFIVLKQNTNFDKIDFTGIASCSELEKKTIILIAIRNKFAHNQLPNKTIYDLAN |
| | EFLKKEKRETYANYYLKVLKKMISDLA |
| IMG_ | LIDRDEFAKWFVEYLENKDYQKFYNSEEYRIRDADFKKNAVIKKQKLKDFYTLQMVNYLLKEVFGKDEM |
| 3300023981_2 | NLQLSELFQTRQERLKLQGIAKKQMNKETGDSSENTRNQTYIWNKDVPVSFFNGKVTIDKVKLKNIGKYK |
| SEQ ID NO: | RYERDERVKTFIGYEVDEKWMMYLPHNWKDRYSVKPINVTDLQIQEYEEIRSHELLKEIQNLEQYIYDHTT |
| 4511 | DKNTLLQDGNPNFKMYVLNGLLTGIKQVNIADFIVLKQNTNFDKIDFTGIASCSELEKKTIILIAIRNKFAHN |
| | QLPNKTIYDLANEFLKKEKRETYANYYLKVLKKMISDLA |
| IMG_ | MENNTTLGKGISYNPYKTADKHYFGGYFNLAMNNIEFVIAEFLTRIGRKETKIANLKKVFTENMSLVDYER |
| 3300027269 | YIHILEEYFPIIKHLDKIHFKINDTVKEVSKEKRITYFIDNFISLLDLTNNLRNFYTHYYHESIAIEENIFDFLDES |
| SEQ ID NO: | LLTTVRDTKENYLKSDKTKQILSISLKQELEILCSEKLNYLKENKIKFNRNDKEALINAVYNDAFKNFLYKK |
| 4512 | GEHFHLTDYKKTKILNPDKLEKDFDLDLSTSGIVYLLSFFLNRKELELFKGNIKGFKASVIRGTSDFEKNSIHF |
| | MATHRIYSVHCYRGLKKKIRSSNHDTKQVLLMQMLDELSKVPHVIYNSLDKELKDTFVEDWNEYFKDNEE |
| | NNENLENSRVIHPVIRKRYEDKFNYFALRFLDNCVDFPTLRFQVHVGDYVHHKMEKSLIDSKIISERIIKEKV |
| | TVFARLDEVNKAKADYFNSLQAENDNRWEFFPNPSYDFPKQNTEKIMGNAKQKNAEKIGIYIQLKNSNLIQ |
| | QTADAKEKLNPHKRSNTKLRKQEIIEKIINLNTDYKSKTPIVHTGEPVAYLSTHDLHSILYDLLIKGETAQAV |
| | EMKIQKQIEKQLREIVDKDTSVKILKKYNKEQTFSNINFSKLQNDLVKERDNLISLLDEHDYRIEDYDRTKK |
| | QRNYPHKRTYILYAAEKGKIAAWLADDIKRFMPKD |
| GCA_ | MENKTSLGNNIYYNPFKPQDKPYFAGYLNAAMENIDSVFRELGKRLKGKEYTSENFFDAIFKENISLVEYER |
| 000827575.1_ | YVKLLSDYFPMARLLDKKEVPIKERKENFKKNFKGIIKAVRDLRNFYTHKEHGEVEITDEIFGVLDEMLKST |
| Cc11.1_ | VLTVKKKKIKTDKTKEILKKSIEKQLDILIKKKLNYLRETAKKVEEKRRIQREMGEEIDPPFRYGNKREDLIA |
| genomic | TIYNDAFDVYIDKKKDSLKESSKAKYNTKSYPQQEEGDLKIPISKNGVVFLLSLFLTKQEIHAFKSKIAGFKA |
| SEQ ID NO: | TVTDEATVSEATVSHRKNSICFMATHEIFSHLAYKKLKRKVRTAEINYGEAENAEQLSVYAKETLMMQML |
| 4513 | DELSKVPDVVYQNLSEDVQKTFIEDWNEYLKENNGDVGTMEEEQVIHPVIRKRYEDKFNYFAIRFLDEFAQ |
| | FPTLRFQVHLGNYLHDSRPKENLISDRRIKEKITVFGRLSELEHKKALFIKNTETNEDREHYWEIFPNPNL |
| GCA_ | MFLERKETEDLKSRVKGFKAKIIKQGEEQISGLKFMATHWVFSYLCFKGIKQKLSTEFHEETLLIQIIDELSK |
| 004119415.1_ | VPDEVYSAFDSKTKEKFVEDINEYMKEGNADLSLEDSKVIHPVIRKRYENKFNYFAIRFLDEYLSSTSLKFQ |
| ASM411941v1_ | VHVGNYVHDRRVKNINGTGFQTERVVKDRVKVFGRLSMISNLKADYIKEQLELPNDSNGWEIFPNPSYIFI |
| genomic_2 | DNNVPIHILADETTKKGIELFKDKRRKEQPEELQKRKGKLSKYNIVSMISKEAKGKDKLRIDEPLALLSLNEI |
| SEQ ID NO: | PALLYQILEKGATPKDIELIIKNKLTERFEKIKNYDPETPAPASQISKRLRNNTTAKGQETLNAEKLSLLIEREI |
| 4514 | EDTETKLSSIEEKRLKAKKEQRRNLPQTSIFLIVTLAV |
| GCA_ | MFLERKETEDLKSRVKGFKAKIIKQGEEQISGLKFMATHWVFSYLCFKGIKQKLSTEFHEETLLIQIIDELSK |
| 004119455.1_ | VPDEVYSAFDSKTKEKFVEDINEYMKEGNADLSLEDSKVIHPVIRKRYENKFNYFAIRFLDEYLSSTSLKFQ |
| ASM411945v1_ | VHVGNYVHDRRVKNINGTGFQTERVVKDRVKVFGRLSMISNLKADYIKEQLELPNDSNGWEIFPNPSYIFI |
| genomic | DNNVPIHILADETTKKGIELFKDKRRKEQPEELQKRKGKLSKYNIVSMISKEAKGKDKLRIDEPLALLSLNEI |
| SEQ ID NO: | PALLYQILEKGATPKDIELIIKNKLTERFEKIKNYDPETPAPASQISKRLRNNTTAKGQETLNAEKLSLLIEREI |
| 4515 | EDTETKLSSIEEKRLKAKKEQRRNLPQTSIFSNSDLGRIAAWLADDIKRFMPAEQRKNWKGYQHSQLQQSL |
| | AYFEKRPQEAFLLLKEGWDTSDGSSYWNNWVMNSFSENNRFEKFYENYLMKRVKYFSELAENIKQHTHN |
| | TKFLRKFIKQQMPADLFPKRHYILKDLETEKIKFYLNH |
| GCA_ | MEKTQTGLGIYYDHTKLQDKYFFGGFFNLAQNNIDNVIKTFILKFFPERKDKDVNAAQFLDICFKDNDADS |
| 003523505.1_ | DFLKKTKFLRMHFPVIGFLASNNDKAGFKRKFSLLLKAISELRNFYTHYYHQPIEFPSELFELLDDIFVETTSE |
| ASM352350v1_ | IKKLKKKDDKTQQLLNKNLSEEYDIRYQQQIERLKELNAQGKKIPLNDETAIRNGVFNAAFNHLIYKDGGD |
| genomic_2 | LKPSRVYQSSYSEPDPAENGTSLSQSSILFLLSMFLERKETEDLKSRVKGFKAKFIKNGEEKISNLKLTATHW |
| SEQ ID NO: | VFSYLCFKGIKQKLSTEFHEETLLIQIIDELSKVPDEVYSAFGAKTKQKFVEDINEYMKEGNADLSLEDSKVI |
| 4516 | HPVIRKRYENKFNYFAIRFLDEYLSSTSLKFQVHVGNYVHDRRIKNINGTDFQTERVVKDSIKVFGRLSKISN |
| | LKADYIKEQLSLPNDSNGWEIFPNPSYVFIDNNVPIHIQTDEATKNGIKLFKDTRRKEQPEELQKRKGKLSKH |
| | NIVEIIFKETKGKDKPRVDEPLALLSLNEIPALLYQILEKGATPEDIELIIKNKLAERFEKIKNYDPETPAPASQI |
| | SKRLRNNTTAKGQETLNAEKLSILIEREIEDTETKLDAIEEKRRKAKKEYRRNSPQKSIFSNSELGRIAAWLA |
| | DDIKRFMPAELRKNWKGYQHSQLQQSLAYFEKRPQEAFLLLKEGWDTSDGSSYWNIWVINSFSETEDFEK |
| | FYENYLRKRAKYFSELAGNIKQHTHNAKFLRKFIKQQMPADLFPKRHYILKDLETEKNKVLSKPLVFSRGL |
| | FDSNPTFIKGVKVTENPELFAEXXNGIATGTKRNIPSSISMAGKETIMSF |
| IMG_ | MNTQPVGLGISYSHTSKNDKHFFGGFLNLGINNLEVLIAAFKLKFFSGDQKKIDIKNFVQTCFTANISDHDFE |
| 3300025944_2 | SRVEFLQNYLPVVRYLDKRNKEGFKNQVELLFKSLDSLRNFYTHYYHAPLSLPQALFDLLDSTFAKVASDV |
| SEQ ID NO: | KANKVKDDKSRHLLKSALSEELNARYKLQLERLKELKASGKKVNLHDHDAIRNGVLNSSFNHLIYKNEAG |
| 4517 | DTIVTRRYAARYSEIESAENGITISQSGLLFLAGLFLKRKEVEDLKSRVKGFKAKIIKEGEENISGLKYMATH |
| | WIFSYLSFKQQNKH |
| UWRZ01.1 | LKASFVKFLKCIYMENHTKQTTYKYDEIADKHYFAGFFNLAWNNIEIVFKVFLKKFKLIEDKDKKIEVNPLS |
| SEQ ID NO: | FVDNYFKNELALSDYRDRIDFLKQYFPVVQYLELLVSKNNDLEKCIGEEKENKRRECFRAKFKSLIRTINEL |
| 4518 | RNYYTHHYHKPIIVDEATFELLDELFLTVVKEVKRYKMKGEPIRHLFKKELNNELTALIKLKKSELETRRKE |
| | GKRVNIDPVSIENAVLNDAFSHLLFGEKGEKFYQSKSTSSNQQSTINISESGLLFLLGMFLHRKESERLRSNIQ |
| | GFKAKVVRDPEKPIDFKNNSLKYMATHWVFNHLAAKPIKERLNTAFQKETLLLQIADELSKVPDEVYQTFS |
| | QEKKNEFLEDINEYFKTGNDIKSFEESRVVHPVIRKRYENKFNYFVLRFLDEFIDFPTLRFQIHLGNYVHDQK |
| | EKPISQGTHLITQRIIKEKINLFGKLSEVTNNKTDFFQKLEVAGGETNLEMFPEPSYNFVGNNIPIYLNLAKSK |
| | VEGAKELNSHLIRLNNEEKKHQKKRTGNKPDKTAILSEIQISDISYGKPVALLSLNELPALLYELLINGKSGE |
| | EIENILVEKLVERYKTINNFSPDNPLPTSQISKKLRKATANERIDIDKLIRAIDREIAVSKEKANLISTKLRDWE |
| | NAKTNRKYAFTKKELGQEATWLADDIKRFMPNKVKENWKGYQHSHLQLLLAFYESRPNEAYSFIQEFWN |
| | LDNDTYLFNRWLKTSFNEKSFHKFYLKYLENRKEYFENIQQQITAFKNQEKLLKKFIEQQHIWSVFYKRLYI |
| | VSPIEEQKRQLLLKPLVFTRGIFDPKPTYIEGKEFEGNKDLFADWYQYIHDEEHVLQKFYSWKRDYKELFEK |
| | FKASDEFTNNKYQLSEKQQF |
| IMG_ | MENHTKQTTYKYDEIADKHYFAGFFNLAWNNIEIVFKVFLKKFKLIEDKDKKIEVNPLSFVDNYFKNELAL |
| 3300025528 | SDYRDRIDFLKQYFPVVQYLELLVSKNNDLEKCIGEEKENKRRECFRAKFKSLIRTINELRNYYTHHYHKPII |
| SEQ ID NO: | VDEATFELLDELFLTVVKEVKRYKMKGEPIRHLFKKELNNELTALIKLKKSELETRRKEGKRVNIDPVSIEN |
| 4519 | AVLNDAFSHLLFGEKGEKFYQSKSTSSNQQSTINISESGLLFLLGMFLHRKESERLRSNIQGFKAKVVRDPEK |
| | PIDFKNNSLKYMATHWVFNHLAAKPIKERLNTAFQKETLLLQIADELSKVPDEVYQTFSQEKKNEFLEDINE |
| | YFKTGNDIKSFEESRVVHPVIRKRYENKFNYFVLRFLDEFIDFPTLRFQIHLGNYVHDQKEKPISQGTHLITQ |
| | RIIKEKINLFGKLSEVTNNKTDFFQKLEVAGGETNLEMFPEPSYNFVGNNIPIYLNLAKSKVEGAKELNSHLI |
| | RLNNEEKKHQKKRTGNKPDKTAILSEIQIS |
| IMG_ | METKQQVGKGISYDHRRPDDKHYFGGFLNLAQNNIDGVIQEFAMRLNREYDPENKNQSLFSYFNINASFTD |
| 3300028733 | WERGVNILKEYWPMMEFIDRPATDKQFEAEKPENREAAKRKYFLATLGALLTSIKDLRHYYTHYYHPPVH |
| SEQ ID NO: | LNDDLFLFLDHALLYTAFDVKKTKMKDDKTRQLLNQNLSLELEKLKKLKVEELKKKKEKGIKVNLQDEK |
| 4520 | GILNAIYNDAFAHIITKEKDSDKDKLETRYKSILPQDEAAETGINISISGLIFLLSLFLSRKEIEQLKSNIEGYKG |
| | KVLNIETEVDRKHNSLKYMATHWVFSILAFKGLKQRLTNSFEKESLLIQMMDELNKVPDELYQTLSETAKK |
| | EFLEDINEYVSEGDDNEKATYVVHPVIRKRYESKFNYFAIRYLDEFAQFPTLKFQIFVGQYLHDNRPKTLAS |
| | NGMTAQRMKEKINLFGNLSEVTKHKSDFFEKESAAQGWEFFPNPSYNWAGNNIYRYDRERRQSQRDTGA |
| | NKQVSQTTQSGTTKR |
| GCA_ | METQQIGKGISYDHLSADDKHYFGGFLNLAQNNIDSVMQEFCSRLNLTYDKRKHKDIINNYFKIHYNPKEK |
| 001897035.1_ | PSHTDWERGVAILKEYWPVVNAIDLPLTAESIKNLPLDEQEKAKREYFTKTLLALFSAIETLRNYYTHYYHP |
| ASM189703v1_ | PITLPESLFVFLDKTLFHTVIDVKKTKMKEDKTRQILKDSLQDQIKKLAELKKNELIEKKKENPRINTNDSEG |
| genomic | ILNSIYNDAFSHFLYTDKDSKKEVLSKWYTSRLPEEKLADSPIGISTSGLVFLLSMFLSRKEVEHLKSNITGY |
| SEQ ID NO: | KGKVLAISEVTKKENGLKFMATHWVFSILAFKGIKHRITSSFEKETFLMQIVDELNKVPDEVYQTLSDGSKK |
| 4521 | TFLEDMNEYVSESVGEDEVPLYVVHPVIRKRYEDKFSYFAIRFLDEX |
| IMG_ | METKEQIGKNIYYAHDIYEDKHYFGAFLNLAQNNIDQVFSEFCTRLNEPKDENIHNIIIKYFSNNVSYSDWD |
| 3300025380 | KRIEILKEYLPVVEYLNLPISDKLFEKYPEKEKEDKRKEYFIKNFQSLIKSVNDLRNFYTHYYHPPVVIDESM |
| SEQ ID NO: | FDFLDSLLLKTCLTVRKKKMKNDKTRQILKKGIIAEWKVLEELKVNELKKNKEKNKWISIDDKEGIRNAIL |
| 4522 | NDSFHHLIFKDKDSFCLKDYHKAKYSKNIFAENKIPISKSGLVFLLSLFLTKKETEQLKANIEGFKAKVIGKE |
| | DEVTKKNNSLKYMATHWVFSYLTYKGLKRRVSTSFDKVTLLTQMLDELSKVPDEVYQTFSISDKDEFLEDI |
| | NEFVQESTGDDKSLIESTVVHPVIRKRYENKFNYFAIRFLDEYANFPTLKFQIFAGLFQHDHKTKNIGESNYI |
| | SDRKIKEKINVFGKLSKVAKYKSDYFTENKNENEWHLFPNPSYNFVGNNIHIYLDMYRKGAEVKSVQEEIN |
| | ALRKIINPKKDRVNRKGKKEIIDMIYNKSSKIEYNEPTALFSLNELPAILYEFLINKKTGEDLENILVQKIVER |
| | YKTIKNYNTTQQLSNSFITKKLRKSSLKQDQINIEKLLRSINKEIEITGEKLNLIKTNKEETTKTNKQDKPERK |
| | YIFYTNELGQEATWLANDLVRFMPKFAKTNWKGYQHSELQRLLAFYDRHKNEAKTLLTTNWDLNSFPIW |
| | GSDINEAFDKDKFDEFYEEYLKKRKKTLEGFANTIELNKNDPKLLKKVLKEVFIAFDKRLFVISSIDKQKNE |
| | LLAKPIVFPRGIFDN |
| CEVJ01.1 | MNETDYLAKRLEYNYASIEDKHYFGGYFNLAQNNINDLSKAFKEKFGMKPKSCILDFFTQDKAIAEYQLG |
| SEQ ID NO: | VEFLQKNLPVIRYLYLPTSHKRFENVPKNQLISEQRNYFKNSLKVLKNLIRDYRNFYTHHFHKPIPVFPETYK |
| 4523 | LLDDLFLAVANDVKKHRMKTDASKQLLKKGLIEELAQLEKLKLEDLKKLKREGKKVNLNDKEAITNAILN |
| | DSFSHLLPKENTISKYYSAVPTEDIDTENGVTISESGIIFLLGLFLTKKQSEDLRSRVKGFKAKLIVNPENPINK |
| | KNNSLKYMATHWVFGYLGFKGLKNRFTTTFTKDTLLAQIVDELSKVPDELYQVLPEELKNEFLEDMNEYL |
| | KEENS |
| IMG_ | MESTVNAKRISYDYKNQEDKHYFGGFLNLAQNNIEETIEALGIRNQVFKKEDSNKKNKSRPAEIIAKVFQID |
| 3300000931 | LKKRKTKDDGSGITYAQWESNVNFLKQYLPIVQFLNLPVSHKKFDHLPKAKKEKAKRDYFIGNFLLLIDIIG |
| SEQ ID NO: | SLRHYYTHYHHKQISIEPELFTLLDEIFLHTCLVVKKRKMKSEKTRELLKRELEREVDILKKLKLAALKKQK |
| 4524 | EDGVRVSLDDEHVERAVLNESFNYLLAKRDNVYKVQPTHCSRGEDGTPFSRSGLVFLVSMFLTKKQGEDF |
| | RSRIKGFKEKIVKREENAISPTNNSLRFMATHWVFSYWSYKGFKAKLNTTFSKEVLELKGI |
| IMG_ | MTQTATTNSGTLADDKQTYYYHFLKSDKFFFGSFFNLADNNLKATFNDFEKRLGIKSANGLVQKVEQYFP |
| 3300027262 | DNLLLSEFERRTELLTEYLPIVHKLRKINKESAEPDRSYFRDNLKMLIKAVDHLRNFYTHYYHKSIIFDERLF |
| SEQ ID NO: | EFLNGALLNVCFDVKKKRMKSDTNKAFLKKHFEENFINKSKDKIKEAFDEAFSHLKVSNDGKKFSLTKFYQ |
| 4525 | AKLSHKQKFSVKNDLIFDITNSDFVFLSNSGLLFLLSFFLRREEQEQLLSKMEGFKNQNELNFIATRWVFTH |
| | KCFKGLKKTIKSSYDKETLLMQMVDELSKCPDVLYKNLSDKQ |
| IMG_ | LSKVPDDVYQAFSEETRNLFVEDINQYLKEGNDDYTLEEAQVIHPVIRKRYENKFNYFAIRYLDEFAGFTSL |
| 3300025944 | KFQVHLGNYIHDKRTKHISGTELQTERRIKERVKVFGKLSDAQRLKNDFFADKSRRDQELGWEILPNPSYV |
| SEQ ID NO: | FIENNIPIYFKVDNEVAEAVKSAKASRKSLSPDERKVRSGDKAQKHIILNSISERGLLRKDEPTALLSLNEIPA |
| 4526 | LLYEILVKGTSPVEIEEILKSKAVERVQVIKNYTPEQPLPGSQISKRLRSNTAVTGKQYNVDKLQQLLKKEIF |
| | LADEKLALIYKNRVELHKKIGGKVLRNYVFGFSELGREATWIAEDIKRFMPLPARREWKGYQHSQLQQSLS |
| | YYESRPNEAFNILKDNWNFDDGAMLWNSWIKDSFNEKFFDRFYERYLHGKRKYLENFLENIQNFSPGSNKI |
| | LEKFLCQQMPKNFFDKRLYVLEPLEQEKDKILSYPLVFPRGLFDPAPTFIKGVQVMEEPERFAAWYRYGYS |
| | PDHPFQRFYEMERDYTDLINDDTETRPDTDKNKSDFSSEQQYALIKKKQDLKIKNIKIQDLFLKLIAETLFSD |
| | IFDYDSEIRLSDLYLTQAERIEKEQNAAQQSIRPAGDDSDNIIKDNFIWSKTIPYIKDQIYEPAVKFKDIGKFK |
| | YFLNDGRINRLLSYDTLKIWSKAEIETEIYIGSASYESIRREAIFKELQKLEEKILARYKGGHPEELEYKNNPS |
| | FKKYIVNGILRKKPDTVSETDCFWLDNFDESTFENPEVFEILSDKLPLVQEAFLLVYLRNKFAHNQLPIKEAY |
| | FYINENYPDLRGSTVSETLLNFLVHAVNNITNRCI |
| IMG_ | MEQEYDFFNKTDKHFFAGLFNTALNNFDLSLAELNKRMNYKEIKGNEKEIIIIEYAFNKDERTQLDFENNFK |
| 3300001348 | YLSESLIFLNRIPSFIAHKNKNGSTIILKDFLKDFLCGLYQTLLNYRNYYTHFEHDDVAIGHPLIAEFLEYLLF |
| SEQ ID NO: | NSVSRVKDDRVKTKAVKDKLLSKYKDDYTTIIEYKNKWICDKNEELINEGRKTFKKINNNSEAGYNYVLN |
| 4527 | SIFRRFIDDSTNTPKLQLDEKCSTDDGLTKVGFIQFLALLLNKRQVSLLFDNITYTRYTDTQLQRVITRWIFTY |
| | ESYRDINYLFKSEYDEHALLLQMVSELTKCPKNLYPYLSEKNKDNFLEDINIYFKENAKLFEDDALVSHEV |
| | VRKRFEDKFPYFAIRFLDEFAKFPSLRFQVNMGKFNHDSREKEFISTGKKTERLILENLTVFENLSEATKKKN |
| | LYFEKSDSKEKSDKESNYKDVSDSIEVSDWVEYPRPKYQFNKNTIGIWLDCDGLGNYDESPKRENKKPTKH |
| | DILDKIELKDSFKKPIAYLSLHELPALLYCLLIEKKDGRFIENRIKGKIRKQRSFLESLKGDYQYSEEELKQFP |
| | KKIRLILTKKSNINSEKIKRQISNEIKVNPLKEIREKYTPKSETELSLSEKGKIATWLSKDIKRFVAKDVKGPSE |
| | EDKNKSWKGYQFSEFQALLSYYDIDKSKLSDFVFKDLNFNINKDFPFQGIVFNKSSLFDFYTHYLKSRREYL |
| | NHLLENFSNTTNEELLLPFKASKFKIKELEEYRKNKLEEPVMLVRGVFDDKPTASREKDKTEFAKWFTVSM |
| | NSSSAQKFYDFDKIYPLTLSVINGRKSEENLTINTKAGLTKQYIP |
| IMG_ | MEQEYDFFNKTDKHFFAGLFNTALNNFDLSLAELNKRMNYKEIKGNEKEIIIIEYAFNKDERTQLDFENNFK |
| 3300025594 | YLSESLIFLNRIPSFIAHKNKNGSTIILKDFLKDFLCGLYQTLLNYRNYYTHFEHDDVAIGHPLIAEFLEYLLF |
| SEQ ID NO: | NSVSRVKDDRVKTKAVKDKLLSKYKDDYTTIIEYKNKWICDKNEELINEGRKTFKKINNNSEAGYNYVLN |
| 4528 | SIFRRFIDDSTNTPKLQLDEKCSTDDGLTKVGFIQFLALLLNKRQVSLLFDNITYTRYTDTQLQRVITRWIFTY |
| | ESYRDINYLFKSEYDEHALLLQMVSELTKCPKNLYPYLSEKNKDNFLEDINIYFKENAKLFEDDALVSHEV |
| | VRKRFEDKFPYFAIRFLDEFAKFPSLRFQVNMGKFNHDSREKEFISTGKKTERLILENLTVFENLSEATKKKN |
| | LYFEKSDSKEKSDKESNYKDVSDSIEVSDWVEYPRPKYQFNKNTIGIWLDCDGLGNY |
| UAMK01.1 | LFETLSAEDQDKFRIEVKDSEEETGSTVLLLRSFDRFPVLALQYLDTMHKFDRIRFQVDLGNYRYKFYEKK |
| SEQ ID NO: | NWIDKADEESADRVRILQKTLTGYGRLNEIEQQRKERWGSLIRAIDQPRADSFDSKPYITDHHASYHLEDN |
| 4529 | HIGLRWNTEGQDILDKSGIFMPSTELPPEADGCMDGTVAPLQAPKCRLSVYDLPAVCFLTYLTGSGKAAED |
| | LIINTTEKYFDFFRALSTGEIIPYNKEAKESFIPLEIKEKIKRCRTEARKTGGQQDQVLSYVIEPYGIDLASLPR |
| | KIQDYLLGDSFLSDGNARFKKLATEKLKKMLEITERKLDTIKETKKVYASKDNKLGKKSHVDIRQGTLARF |
| | LAKDMVFFKRPDPQGRIMLTSQNFDILQKELALFSKPLRGLKQLFITAELIGCKYPEENHPFLQKVLDRNPS |
| | GFLDFYIAYLSERRKYLEGILMSKQNDYSQYHFLHPERAKWSNRNRDYYNKLAARYTTIELPGNLFLEAIV |
| | KELKGIDQNKLQYPQTLSDALAQERKNVAFLINAYMKAVGEGCQPFYNYKRGYRYFSMTCKPDWDFSKPI |
| | EKLKDKYLTVGQMEQFMSDNDKEARESFYLRSLDARNAAKVTKAKNQGRYDSRKRGYLKDELEASKVE |
| | APEKLSHSLKFYKENEKEIRRIKVQDAVLYLLAKDVLTHTMDNADLSAYKLKYIGKDNDTDILSMQLPFAV |
| | RLQIRTSDDSTKEVTIRQEDLKLKNYGDFFSFIYDSRIRPLLAQVDAELIDRSQLEKELDNYDRKRVPLFEYV |
| | HNLESRVCETLNEEQFHKDAEGNPVKMDFKYLLRYLNISEKTEDLLKAIRNAFCHGTYPEGSRVTLVFEKE |
| | DCLLYTSDAA |
| GCA_ | MPAEQRKNWKGYQHSQLQQSLAYFEKRPQEAFLLLKEGWDTSDGSSYWNNWVMNSFSENNRFEKFYEN |
| 004119415.1_ | YLMKRVKYFSELAENIKQHTHNTKFLRKFIKQQMPADLFPKRHYILKDLETEKNKVLSKPLVFSRGLFDSN |
| ASM411941v1_ | PTFIKGVKVTENPELFAEWYSYGYKTEHTFQHFYGWERDYNELLDNELQKGNSFAKNSIHYSRESQLDLIK |
| genomic | LKQDLKIKKIKIQDLFLKRIAEKLFENVFNYTTTLSLDEFYMTQEERAEKERIALAQSQREEGDKSSNIIKDN |
| SEQ ID NO: | FIWSKTIAFESQQIYELAIKLKDLGKFNRFLLDHKVLTLLSYDQNKIWNKEQLERELSIGENSYEVIRREKLF |
| 4530 | KEIQNLELQTLSNWSWDGINHPREFEMEDQKNARHPNFKMYLVNGILRKNTNFYKEGEDFWLESLKENDF |
| | KTLPSEILETKSEMVQLLFLVIMIRNQFAHNQLPKVQLYNFIRKNYPEIQNNTAAELYLNLIKLAVQKLKENS |
| GCA_ | LFDSNPTFIKGVKVTENPELFAEWYSYGYKTEHTFQHFYGWERDYNELLDNELQKGNSFAKNSIHYSRESQ |
| 004119455.1_ | LDLIKLKQDLKIKKIKIQDLFLKRIAEKLFENVFNYTTTLSLDEFYMTQEERAEKERIALAQSQREEGDKSSNI |
| ASM411945v1_ | IKDNFIWSKTIAFESQQIYELAIKLKDLGKFNRFLLDHKVLTLLSYDQNKIWNKEQLERELSIGENSYEVIRR |
| genomic_2 | EKLFKEIQNLELQTLSNWSWDGINHPREFEMEDQKNARHPNFKMYLVNGILRKNTNFYKEGEDFWLESLK |
| SEQ ID NO: | ENDFKTLPSEILETKSEMVQLLFLVIMIRNQFAHNQLPKVQLYNFIRKNYPEIQNNTAAELYLNLIKLAVQKL |
| 4531 | KENS |
| GCA_ | MXEWYSYGYKTEHTFQHFYGWERDYNELLDNELQKDNSFAKNSIHYSRESQLDLIKLKQDLKIKKIKIQDL |
| 003523505.1_ | FLKRIAEKLFENVFHYPTTLSLDEFYMTQEERAEKERIALAQSLREEGDNSPNIIKDNFIWSKTIAFESQQISEP |
| ASM352350v1_ | AIKLKDIGKFNRFLLDSKVKTLLSYDQNKKDKEQLERELSIGENSYEVIRREKLFKEIQNLELQTLSNWPWD |
| genomic | GINHPREFEMEDQKNIWHPNFKMYVVNGILRKNSNFYKEDEDFWLESLKENDFKTLPSEILETKSEMVQLL |
| SEQ ID NO: | FLVIMIRNQFAHNQLPEVQFYNFIRKNYPEIQNNTAAELYLNLIKLAVQKLKENS |
| 4532 | |
| GCA_ | MARSTKFTKSMFSYESSFKRFSHRKGMQSGFLKSTPSKSNPYSYNYKPINGYKDYRLDSLINNQTDLWSKY |
| 000212915.1_ | SRKQDKFMLYASRYLAESNYFGEEAMFKVYQFASNEEQEKYIVEAKQNLPKREYDKLKYHKGRLVVYKS |
| ASM21291v1_ | YHNHLQEYPRWDYPFVVENNAIQIYVKILGEPWIVSIQRRLIIYFLEDALFSKKKESNGIALLQNYLPHHQRD |
| genomic | VRNGLFVFKTGQTNNLSTKEMSNLRKLFPRKLIQSYLYEDNTGDMDSPSQVLSDTSINDTEKKGTKKILNL |
| SEQ ID NO: | RVGKHLKLRYIRKVWNLIYFKDIYKDKAQRMGHHKKFHITKDEFVFYTRWMYSFESIPSYKDHLIQFFIKK |
| 4533 | HFFNNEEFKELFLNSSSIDELYLQTKRNFIKWSAHNVNSEKKEKTYSLEDYKLFFESKILYINVSHFISFLNQE |
| | KVIQKNDNGIIQYKALKNLSYLIKPFYYKDKLEIEHYKTYGKVFNKLRSIKLEDCLLYEIAYRYLLNVTPSFP |
| | KYKQLIIQSFPKEKVDLLVNAIYSFEIHNKKGAFIYSIQVPFLKLNELVCLIYRNSTKIAATNKEFLFLQIYKYL |
| | VNYCKNKPVDYELYTVCYKFNQLKVLGYDDLLHFLKHIVKRGLQLTQILTQLEKFLIIKNNIQIDIQKQGTL |
| | NSLSIYECSKMNNPQKLEDLRIKAINFDIPDTDYPSILEHIEKQFIIKETPFKPVSWSHLEKHTQDMCDIMMN |
| | MLHLNLYKRNSDTESREEAKIQFRDRYFNTVVKQSD |
| IMG_ | MPVSNHQQKGHKTYFTNHSNKAEISVFVNTALNNIYRIIKTIEENVFQAVPDYNREEMYKSQVFTVLFSKR |
| 3300009446 | NEVKKERIIQYLTRWLPWFSKGLTITDPRRFALRLVVYIQKLVELRNFYSHSLKNVVNLNLYTNAHVPSSA |
| SEQ ID NO: | KDVFIDLVCDIRREERDKKDSFIPFDHVKAEYKDYMFDAILHEDLNREKYACYEQDYCNFRADFKQLYKST |
| 4534 | KNEVRERFKKERNLNDEKLKKQGVIFSAGKGPDSKQNANKLTEFGLVFFISIFLERRMVADFLDTVYPNDIG |
| | FMDKLVKRSLTIYNAKPPKEQLISMDRKFALGLDILNMLNRVPNYIYDHLTDGAKEKAIDEEGIVMKRYND |
| | RFPYLILQCLEYSGKLEGLQLMCLIGKNFNAKPYHKQFEGKSEIREIHKTLYAFDHLSDIRENDKYYQELRS |
| | KDHEVNYNIGEEELAIINEIKNLHTYPLYQYYPKYGIHEYSTWKYIGFVFQDESIGPPVIAQSEESETRIVITPE |
| | FRVNNTQHQFTLDQSLLKYLAYLLKGESTVSENENGLASFKDLCLQFKSDFVRLLNDIRDQNITPDSDYDY |
| | SQILNSYNIPSACIPKRIKKYLNAKQSSNNSRKHIKTKLEYMLCETKCLLAENPVRSKPLPKEEYINRKKESQ |
| | YFLMRGDKATWITNDILFFMKPKLVSIEKEGQKTNHFHKLNNQQAKILQSKLALMDHNFHDIRSFMKETG |
| | VFETGSEHIFLTEQNIKAKYQKVDNFFVRYLGLRKAYLESTIKKLKTKNQIINQTELERAYYYIKSKTIRRSV |
| | ANEDKHIAITTYIENLKKQPIIIHPELMKKWANDVYQKEESENNQVHNLSYIVNDLDESFARYKQQWFYQK |
| | DSLIDGFGKRPQFQKRP |
| IMG_ | MHQKKQQKQKKKQSRRAKELTLKERSAYAIAANLAQSRVEHILEGDESPNSLNKLYDKITGHLREEIQRYY |
| 3300027338 | GKDENNKDERALIMESALDLLNRLRNYYSHILYDDPGDVSFMLKGSEEGQNKKQDEENGDRPLISWLTWL |
| SEQ ID NO: | YREAWKKQDLEEKFPLWDSVDDNISRLSHYGAAFFINLFLTRSKAEHFLQRLGKFEGKNKKRSRHVFSAYC |
| 4535 | QRDRISDTFIQDPPEHMLYREIMGALKIPPFHSKAGQENKKKVEDSKYEQPPEYSDTDVLPFRSQSRARDYV |
| | LQLIDMLGLLPNIRFRGIVETKEIDKEGGIIWQPAHEIIKSKVKKKSQDEKGDKKSYDRDKRVIRKTYNRNN |
| | EALKEALKEVGQRFVYDHSDGNILFEIEQKGKDPVRGVVRWRDFLTWVYLIAFEKKPSNKIDEEIYGYLSG |
| | YKETLSEGKTPKEVYK |
| UYAX01.1_2 | VRECNEDEKFTAWKNGFISEMLQSTKNRIKRFEKDSEAVISSDNKPGKKNHVSLKPGAYASFIANDIVFFQE |
| SEQ ID NO: | CGATEKMTGLNFKVMQSRLATFTKDGSTSFNILLQTLKNAHLVSTTYGKGDHPFLYRVIKQQPSDIVQFYK |
| 4536 | IYLNEKVLYLQSDIPDNAIFLHGERKRWENRNEQYYRDLAERYLQRPIQLPRQLFESHIRQLLLSDCIKGERG |
| | NDLKEAINSAASQGRCNTTYMIMEYFADYLCDGTQFFYGLFDGDLSHEYNYRFYSLISNNIENSKKLVLTL |
| | KKGNNSKESPFISALERGIHWSKMNPLMKKGLKNDSSEGDFVHAAKRAYKEMTETERMFRRYAVQDEVL |
| | FLAAKITIRRVLGLSEQYNCLLGDIKPQGGSLLEQTIPSITTKHTINTGNKKQKPKQVQILQKNVKLKDFGKV |
| | FKLLNDRRIFDLLFNKGNEAVSMTDLCEELERYDRHRVDVFDSVLKYESKITKGYTNKELMNESGQIDFKA |
| | IQAFDKQNTTADKEDLRLIRNAFSHNQYPQYNNEPILFDKDIPEIADEISIIAKDIEENTK |
| OLVX01.1 | LKKCPFELYELLGSEDKRLFTIVADTGETILLRRHEDRFPQLALSWIDSSKAFDHLRFQVNAGKLRYLFRDN |
| SEQ ID NO: | KHCIDGQTRMRVLEEPLNGYRRLMEFEEERIQKQSGEIRSLWPGLDILNKDETPRNDASVLPYISDYRVRYL |
| 4537 | FDGDNIGISIGDFTPSITKTDETKYRVTGKTADCCLSKYELPGLLFYHLLTLRHGDNKRSTKNAEDIIIDAIKR |
| | YKRLFSDVKEGILKPIKEENANQLGNRIWNSYGINIKDIPDKIIDYLLVRECNEDEKFTAWKNGFISEMLQST |
| | KNRIKRFEKDSEAVISSDNKPGKKNHVSLKPGAYASFIANDIVFFQECGATEKMTGLNFKVMQSRLATFTK |
| | DGSTSFNILLQTLKNAHLVSTTYGKGDHPFLYRVIKQQPSDIVQFYKIYLNEKVLYLQSDIPDNAIFLHGERK |
| | RWENRNEQYYRDLAERYLQRPIQLPRQLFESHIRQLLLSDCIKGERGNDLKEAINSAASQGRCNTTYMIME |
| | YFADYLCDGTQFFYGLFDGDLSHEYNYQFYSLISNNIENSKKLVLTLKKGNNSKESPFISALERGIHWSKMN |
| | PLMKKGLKNDSSEGDFVHAAKRAYKEMTETERMFRRYAVQDEVLFLAAKITIRRVLGLSEQYNCLLGDIK |
| | PQGGSLLEQTIPSITTKHTINTGNKKQKPKQVQILQKNVKLKDFGKVFKLLNDRRIFDLLFNKGNEAVSMTD |
| | LCEELERYDRHRVDVFDSVLKYESKITKGYTNKELMNESGQIDFKAIQAFDKQNTTADKEDLRLIRNAFSH |
| | NQYPQYNNEPILFDKDIPEIADEISIIAKDIEENTK |
| IMG_ | LPAVINYKPELSTLAIILEKATQSEDRYNRLLKKAEDEGNYADFIKRNKGKQFKLQFIRKAWHLMYFKNSY |
| 3300028862 | TQQLESTGKHHKNFHITRDEFNDFCRYMFAFDEVPAYKNYLREMLDKKQFFKNDQFKILFENGDSLDSLYS |
| SEQ ID NO: | KTKQSYEKWLQGQSTKEQETEKYTLSNYENIFQDKMLYVNVSHFTGFLKTTGIWTENEHGVIQFKALENR |
| 4538 | RYLIQEYYYADKLEKPEYKNCRKLFNELKTVKLEDALLYEIAMRYLQIDSQIVQNVRTSIIEILNQNIRFLIK |
| | NKENKALYELIVPFKKIDSYVGLLAHKKEQEMDPKSKGSSFLTNIAGYLELVKDHKDLKKVYGSFTANKN |
| | MPVLTFDDLHKIDAHLITHSIRFTNLALAMEHYFVVKKNISIVKDNRITYDEIKDLKPYFDNKTRNKAFHFG |
| | VPSKSYETFIREVEQKFLFNEVKTTKPTSFQSLSRQHKIMCGMFLELIHNDLYNKGEKDSKKKRNDAEASYF |
| | NSVISK |
| IMG_ | LPAVINYKPELSTLAIILEKATQSEDRYNRLLKKAEDEGNYADFIKRNKGKQFKLQFIRKAWHLMYFKNSY |
| 3300028767 | TQQLESTGKHHKNFHITRDEFNDFCRYMFAFDEVPAYKNYLREMLDKKQFFKNDQFKILFENGDSLDSLYS |
| SEQ ID NO: | KTKQSYEKWLQGQSTKEQETEKYTLSNYENIFQDKMLYVNVSHFTGFLKTTGIWTENEHGVIQFKALENR |
| 4539 | RYLIQEYYYADKLEKPEYKNCRKLFNELKTVKLEDALLYEIAMRYLQIDSQIVQNVRTSIIEILNQNIRFLIK |
| | NKENKALYELIVPFKKIDSYVGLLAHKKEQEMDPKSKGSSFLTNIAGYLELVKDHKDLKKVYGSFTANKN |
| | MPVLTFDDLHKIDAHLITHSIRFTNLALAMEHYFVVKKNISIVKDNRITYDEIKDLKPYFDNKTRNKAFHFG |
| | VPSKSYETFIREVEQKFLFNEVKTTKPTSFQSLSRQHKIMCGMFLELIHNDLYNKGEKDSKKKRNDAEASYF |
| | NSVISK |
| IMG_ | LPAVINYKPELSTLAIILEKATQSEDRYNRLLKKAEDEGNYADFIKRNKGKQFKLQFIRKAWHLMYFKNSY |
| 3300028738_3 | TQQLESTGKHHKNFHITRDEFNDFCRYMFAFDEVPAYKNYLREMLDKKQFFKNDQFKILFENGDSLDSLYS |
| SEQ ID NO: | KTKQSYEKWLQGQSTKEQETEKYTLSNYENIFQDKMLYVNVSHFTGFLKTTGIWTENEHGVIQFKALENR |
| 4540 | RYLIQEYYYADKLEKPEYKNCRKLFNELKTVKLEDALLYEIAMRYLQIDSQIVQNVRTSIIEILNQNIRFLIK |
| | NKENKALYELIVPFKKIDSYVGLLAHKKEQEMDPKSKGSSFLTNIAGYLELVKDHKDLKKVYGSFTANKN |
| | MPVLTFDDLHKIDAHLITHSIRFTNLALAMEHYFVVKKNISIVKDNRITYDEIKDLKPYFDNKTRNKAFHFG |
| | VPSKSYETFIREVEQKFLFNEVKTTKPTSFQSLSRQHKIMCGMFLELIHNDLYNKGEKDSKKKRNDAEASYF |
| | NSVISK |
| IMG_ | LPAVINYKPELSTLAIILEKATQSEDRYNRLLKKAEDEGNYADFIKRNKGKQFKLQFIRKAWHLMYFKNSY |
| 3300030943_4 | TQQLESTGKHHKNFHITRDEFNDFCRYMFAFDEVPAYKNYLREMLDKKQFFKNDQFKILFENGDSLDSLYS |
| SEQ ID NO: | KTKQSYEKWLQGQSTKEQETEKYTLSNYENIFQDKMLYVNVSHFTGFLKTTGIWTENEHGVIQFKALENR |
| 4541 | RYLIQEYYYADKLEKPEYKNCRKLFNELKTVKLEDALLYEIAMRYLQIDSQIVQNVRTSIIEILNQNIRFLIK |
| | NKENKALYELIVPFKKIDSYVGLLAHKKEQEMDPKSKGSSFLTNIAGYLELVKDHKDLKKVYGSFTANKN |
| | MPVLTFDDLHKIDAHLITHSIRFTNLALAMEHYFVVKKNISIVKDNRITYDEIKDLKPYFDNKTRNKAFHFG |
| | VPSKSYETFIREVEQKFLFNEVKTTKPTSFQSLSRQHKIMCGMFLELIHNDLYNKGEKDSKKKRNDAEASYF |
| | NSVISK |
| IMG_ | LPAVINYKPELSTLAIILEKATQSEDRYNRLLKKAEDEGNYADFIKRNKGKQFKLQFIRKAWHLMYFKNSY |
| 3300031521 | TQQLESTGKHHKNFHITRDEFNDFCRYMFAFDEVPAYKNYLREMLDKKQFFKNDQFKILFENGDSLDSLYS |
| SEQ ID NO: | KTKQSYEKWLQGQSTKEQETEKYTLSNYENIFQDKMLYVNVSHFTGFLKTTGIWTENEHGVIQFKALENR |
| 4542 | RYLIQEYYYADKLEKPEYKNCRKLFNELKTVKLEDALLYEIAMRYLQIDSQIVQNVRTSIIEILNQNIRFLIK |
| | NKENKALYELIVPFKKIDSYVGLLAHKKEQEMDPKSKGSSFLTNIAGYLELVKDHKDLKKVYGSFTANKN |
| | MPVLTFDDLHKIDAHLITHSIRFTNLALAMEHYFVVKKNISIVKDNRITYDEIKDLKPYFDNKTRNKAFHFG |
| | VPSKSYETFIREVEQKFLFNEVKTTKPTSFQSLSRQHKIMCGMFLELIHNDLYNKGEKDSKKKRNDAEASYF |
| | NSVISK |
| IMG_ | LPAVINYKPELSTLAIILEKATQSEDRYNRLLKKAEDEGNYADFIKRNKGKQFKLQFIRKAWHLMYFKNSY |
| 3300031918_3 | TQQLESTGKHHKNFHITRDEFNDFCRYMFAFDEVPAYKNYLREMLDKKQFFKNDQFKILFENGDSLDSLYS |
| SEQ ID NO: | KTKQSYEKWLQGQSTKEQETEKYTLSNYENIFQDKMLYVNVSHFTGFLKTTGIWTENEHGVIQFKALENR |
| 4543 | RYLIQEYYYADKLEKPEYKNCRKLFNELKTVKLEDALLYEIAMRYLQIDSQIVQNVRTSIIEILNQNIRFLIK |
| | NKENKALYELIVPFKKIDSYVGLLAHKKEQEMDPKSKGSSFLTNIAGYLELVKDHKDLKKVYGSFTANKN |
| | MPVLTFDDLHKIDAHLITHSIRFTNLALAMEHYFVVKKNISIVKDNRITYDEIKDLKPYFDNKTRNKAFHFG |
| | VPSKSYETFIREVEQKFLFNEVKTTKPTSFQSLSRQHKIMCGMFLELIHNDLYNKGEKDSKKKRNDAEASYF |
| | NSVISK |
| IMG_ | LPAVINYKPELSTLAIILEKATQSEDRYNRLLKKAEDEGNYADFIKRNKGKQFKLQFIRKAWHLMYFKNSY |
| 3300029989_4 | TQQLESTGKHHKNFHITRDEFNDFCRYMFAFDEVPAYKNYLREMLDKKQFFKNDQFKILFENGDSLDSLYS |
| SEQ ID NO: | KTKQSYEKWLQGQSTKEQETEKYTLSNYENIFQDKMLYVNVSHFTGFLKTTGIWTENEHGVIQFKALENR |
| 4544 | RYLIQEYYYADKLEKPEYKNCRKLFNELKTVKLEDALLYEIAMRYLQIDSQIVQNVRTSIIEILNQNIRFLIK |
| | NKENKALYELIVPFKKIDSYVGLLAHKKEQEMDPKSKGSSFLTNIAGYLELVKDHKDLKKVYGSFTANKN |
| | MPVLTFDDLHKIDAHLITHSIRFTNLALAMEHYFVVKKNISIVKDNRITYDEIKDLKPYFDNKTRNKAFHFG |
| | VPSKSYETFIREVEQKFLFNEVKTTKPTSFQSLSRQHKIMCGMFLELIHNDLYNKGEKDSKKKRNDAEASYF |
| | NSVISK |
| IMG_ | LPAVINYKPELSTLAIILEKATQSEDRYNRLLKKAEDEGNYADFIKRNKGKQFKLQFIRKAWHLMYFKNSY |
| 3300029998_5 | TQQLESTGKHHKNFHITRDEFNDFCRYMFAFDEVPAYKNYLREMLDKKQFFKNDQFKILFENGDSLDSLYS |
| SEQ ID NO: | KTKQSYEKWLQGQSTKEQETEKYTLSNYENIFQDKMLYVNVSHFTGFLKTTGIWTENEHGVIQFKALENR |
| 4545 | RYLIQEYYYADKLEKPEYKNCRKLFNELKTVKLEDALLYEIAMRYLQIDSQIVQNVRTSIIEILNQNIRFLIK |
| | NKENKALYELIVPFKKIDSYVGLLAHKKEQEMDPKSKGSSFLTNIAGYLELVKDHKDLKKVYGSFTANKN |
| | MPVLTFDDLHKIDAHLITHSIRFTNLALAMEHYFVVKKNISIVKDNRITYDEIKDLKPYFDNKTRNKAFHFG |
| | VPSKSYETFIREVEQKFLFNEVKTTKPTSFQSLSRQHKIMCGMFLELIHNDLYNKGEKDSKKKRNDAEASYF |
| | NSVISK |
| IMG_ | LPAVINYKPELSTLAIILEKATQSEDRYNRLLKKAEDEGNYADFIKRNKGKQFKLQFIRKAWHLMYFKNSY |
| 3300030339_3 | TQQLESTGKHHKNFHITRDEFNDFCRYMFAFDEVPAYKNYLREMLDKKQFFKNDQFKILFENGDSLDSLYS |
| SEQ ID NO: | KTKQSYEKWLQGQSTKEQETEKYTLSNYENIFQDKMLYVNVSHFTGFLKTTGIWTENEHGVIQFKALENR |
| 4546 | RYLIQEYYYADKLEKPEYKNCRKLFNELKTVKLEDALLYEIAMRYLQIDSQIVQNVRTSIIEILNQNIRFLIK |
| | NKENKALYELIVPFKKIDSYVGLLAHKKEQEMDPKSKGSSFLTNIAGYLELVKDHKDLKKVYGSFTANKN |
| | MPVLTFDDLHKIDAHLITHSIRFTNLALAMEHYFVVKKNISIVKDNRITYDEIKDLKPYFDNKTRNKAFHFG |
| | VPSKSYETFIREVEQKFLFNEVKTTKPTSFQSLSRQHKIMCGMFLELIHNDLYNKGEKDSKKKRNDAEASYF |
| | NSVISK |
| IMG_ | MDTNEAYTAYNSRNSFKRIFDFKGEIAPIAEKANLNYDIKAKNAINREQRLHYFTVGHTFKNIDTEHVFEILL |
| 3300028862_2 | DEETREKRPYTFLSLQQFNTDFCTAIKEVISNIRHINSHYIHDFERIKTDNIPPEIITFLKESFELAVIQIYLKENN |
| SEQ ID NO: | ITYLQFIEQKNTDTTIVKYLHDKFYSLDNTKTDTKNDTSPSLAEYIAFRNTFKTLSKEKALDSLLFVTVDAGF |
| 4547 | PWKLEETHTACTITQGTYLSFNACLFLLSLFLYKSEANQLISKIKGFKKNKTDEEKSKREIFSFFSKKFSSQDI |
| | DSEENHLVKFRDLIQYINHYPVEWNKDLKLESGHPLMTDKLIAKITDMEIDRAYPDYAGNNKFQAYAKEL |
| | LWNVPSKTTFTTEEIEAFAFEINKSPELKDAKKKLHDLQAKMGLYGFKKVKNEQEIAKTIKRIKWIQNDLNP |
| | VTENVKKRLAQFSLYGSYGRNQDRFMDFATRYLAEQKYFGVDAEFKMYKYFTSEEQNTELATYELPKDK |
| | KAYDKLRFHKGKLVHFSTFENHLKKYESWDTPFVIENNAIQVKLSIRQDNKKEPIEKILSIQRALMLYFLED |
| | ALFQTGNNNIIENKGRILVEQYYTVYNNDFVQSKTVLEENDSISPEQKNALKKIVPKRLLHRYFARSNKL |
| IMG_ | MDTNEAYTAYNSRNSFKRIFDFKGEIAPIAEKANLNYDIKAKNAINREQRLHYFTVGHTFKNIDTEHVFEILL |
| 3300028767_2 | DEETREKRPYTFLSLQQFNTDFCTAIKEVISNIRHINSHYIHDFERIKTDNIPPEIITFLKESFELAVIQIYLKENN |
| SEQ ID NO: | ITYLQFIEQKNTDTTIVKYLHDKFYSLDNTKTDTKNDTSPSLAEYIAFRNTFKTLSKEKALDSLLFVTVDAGF |
| 4548 | PWKLEETHTACTITQGTYLSFNACLFLLSLFLYKSEANQLISKIKGFKKNKTDEEKSKREIFSFFSKKFSSQDI |
| | DSEENHLVKFRDLIQYINHYPVEWNKDLKLESGHPLMTDKLIAKITDMEIDRAYPDYAGNNKFQAYAKEL |
| | LWNVPSKTTFTTEEIEAFAFEINKSPELKDAKKKLHDLQAKMGLYGFKKVKNEQEIAKTIKRIKWIQNDLNP |
| | VTENVKKRLAQFSLYGSYGRNQDRFMDFATRYLAEQKYFGVDAEFKMYKYFTSEEQNTELATYELPKDK |
| | KAYDKLRFHKGKLVHFSTFENHLKKYESWDTPFVIENNAIQVKLSIRQDNKKEPIEKILSIQRALMLYFLED |
| | ALFQTGNNNIIENKGRILVEQYYTVYNNDFVQSKTVLEENDSISPEQKNALKKIVPKRLLHRYFARSNKL |
| IMG_ | MDTNEAYTAYNSRNSFKRIFDFKGEIAPIAEKANLNYDIKAKNAINREQRLHYFTVGHTFKNIDTEHVFEILL |
| 3300028738_4 | DEETREKRPYTFLSLQQFNTDFCTAIKEVISNIRHINSHYIHDFERIKTDNIPPEIITFLKESFELAVIQIYLKENN |
| SEQ ID NO: | ITYLQFIEQKNTDTTIVKYLHDKFYSLDNTKTDTKNDTSPSLAEYIAFRNTFKTLSKEKALDSLLFVTVDAGF |
| 4549 | PWKLEETHTACTITQGTYLSFNACLFLLSLFLYKSEANQLISKIKGFKKNKTDEEKSKREIFSFFSKKFSSQDI |
| | DSEENHLVKFRDLIQYINHYPVEWNKDLKLESGHPLMTDKLIAKITDMEIDRAYPDYAGNNKFQAYAKEL |
| | LWNVPSKTTFTTEEIEAFAFEINKSPELKDAKKKLHDLQAKMGLYGFKKVKNEQEIAKTIKRIKWIQNDLNP |
| | VTENVKKRLAQFSLYGSYGRNQDRFMDFATRYLAEQKYFGVDAEFKMYKYFTSEEQNTELATYELPKDK |
| | KAYDKLRFHKGKLVHFSTFENHLKKYESWDTPFVIENNAIQVKLSIRQDNKKEPIEKILSIQRALMLYFLED |
| | ALFQTGNNNIIENKGRILVEQYYTVYNNDFVQSKTVLEENDSISPEQKNALKKIVPKRLLHRYFARSNKL |
| IMG_ | MDTNEAYTAYNSRNSFKRIFDFKGEIAPIAEKANLNYDIKAKNAINREQRLHYFTVGHTFKNIDTEHVFEILL |
| 3300030047_3 | DEETREKRPYTFLSLQQFNTDFCTAIKEVISNIRHINSHYIHDFERIKTDNIPPEIITFLKESFELAVIQIYLKENN |
| SEQ ID NO: | ITYLQFIEQKNTDTTIVKYLHDKFYSLDNTKTDTKNDTSPSLAEYIAFRNTFKTLSKEKALDSLLFVTVDAGF |
| 4550 | PWKLEETHTACTITQGTYLSFNACLFLLSLFLYKSEANQLISKIKGFKKNKTDEEKSKREIFSFFSKKFSSQDI |
| | DSEENHLVKFRDLIQYINHYPVEWNKDLKLESGHPLMTDKLIAKITDMEIDRAYPDYAGNNKFQAYAKEL |
| | LWNVPSKTTFTTEEIEAFAFEINKSPELKDAKKKLHDLQAKMGLYGFKKVKNEQEIAKTIKRIKWIQNDLNP |
| | VTENVKKRLAQFSLYGSYGRNQDRFMDFATRYLAEQKYFGVDAEFKMYKYFTSEEQNTELATYELPKDK |
| | KAYDKLRFHKGKLVHFSTFENHLKKYESWDTPFVIENNAIQVKLSIRQDNKKEPIEKILSIQRALMLYFLED |
| | ALFQTGNNNIIENKGRILVEQYYTVYNNDFVQSKTVLEENDSISPEQKNALKKIVPKRLLHRYFARSNKL |
| IMG_ | MDTNEAYTAYNSRNSFKRIFDFKGEIAPIAEKANLNYDIKAKNAINREQRLHYFTVGHTFKNIDTEHVFEILL |
| 3300030943_5 | DEETREKRPYTFLSLQQFNTDFCTAIKEVISNIRHINSHYIHDFERIKTDNIPPEIITFLKESFELAVIQIYLKENN |
| SEQ ID NO: | ITYLQFIEQKNTDTTIVKYLHDKFYSLDNTKTDTKNDTSPSLAEYIAFRNTFKTLSKEKALDSLLFVTVDAGF |
| 4551 | PWKLEETHTACTITQGTYLSFNACLFLLSLFLYKSEANQLISKIKGFKKNKTDEEKSKREIFSFFSKKFSSQDI |
| | DSEENHLVKFRDLIQYINHYPVEWNKDLKLESGHPLMTDKLIAKITDMEIDRAYPDYAGNNKFQAYAKEL |
| | LWNVPSKTTFTTEEIEAFAFEINKSPELKDAKKKLHDLQAKMGLYGFKKVKNEQEIAKTIKRIKWIQNDLNP |
| | VTENVKKRLAQFSLYGSYGRNQDRFMDFATRYLAEQKYFGVDAEFKMYKYFTSEEQNTELATYELPKDK |
| | KAYDKLRFHKGKLVHFSTFENHLKKYESWDTPFVIENNAIQVKLSIRQDNKKEPIEKILSIQRALMLYFLED |
| | ALFQTGNNNIIENKGRILVEQYYTVYNNDFVQSKTVLEENDSISPEQKNALKKIVPKRLLHRYFARSNKL |
| IMG_ | MDTNEAYTAYNSRNSFKRIFDFKGEIAPIAEKANLNYDIKAKNAINREQRLHYFTVGHTFKNIDTEHVFEILL |
| 3300031521_2 | DEETREKRPYTFLSLQQFNTDFCTAIKEVISNIRHINSHYIHDFERIKTDNIPPEIITFLKESFELAVIQIYLKENN |
| SEQ ID NO: | ITYLQFIEQKNTDTTIVKYLHDKFYSLDNTKTDTKNDTSPSLAEYIAFRNTFKTLSKEKALDSLLFVTVDAGF |
| 4552 | PWKLEETHTACTITQGTYLSFNACLFLLSLFLYKSEANQLISKIKGFKKNKTDEEKSKREIFSFFSKKFSSQDI |
| | DSEENHLVKFRDLIQYINHYPVEWNKDLKLESGHPLMTDKLIAKITDMEIDRAYPDYAGNNKFQAYAKEL |
| | LWNVPSKTTFTTEEIEAFAFEINKSPELKDAKKKLHDLQAKMGLYGFKKVKNEQEIAKTIKRIKWIQNDLNP |
| | VTENVKKRLAQFSLYGSYGRNQDRFMDFATRYLAEQKYFGVDAEFKMYKYFTSEEQNTELATYELPKDK |
| | KAYDKLRFHKGKLVHFSTFENHLKKYESWDTPFVIENNAIQVKLSIRQDNKKEPIEKILSIQRALMLYFLED |
| | ALFQTGNNNIIENKGRILVEQYYTVYNNDFVQSKTVLEENDSISPEQKNALKKIVPKRLLHRYFARSNKL |
| IMG_ | MDTNEAYTAYNSRNSFKRIFDFKGEIAPIAEKANLNYDIKAKNAINREQRLHYFTVGHTFKNIDTEHVFEILL |
| 3300031918_4 | DEETREKRPYTFLSLQQFNTDFCTAIKEVISNIRHINSHYIHDFERIKTDNIPPEIITFLKESFELAVIQIYLKENN |
| SEQ ID NO: | ITYLQFIEQKNTDTTIVKYLHDKFYSLDNTKTDTKNDTSPSLAEYIAFRNTFKTLSKEKALDSLLFVTVDAGF |
| 4553 | PWKLEETHTACTITQGTYLSFNACLFLLSLFLYKSEANQLISKIKGFKKNKTDEEKSKREIFSFFSKKFSSQDI |
| | DSEENHLVKFRDLIQYINHYPVEWNKDLKLESGHPLMTDKLIAKITDMEIDRAYPDYAGNNKFQAYAKEL |
| | LWNVPSKTTFTTEEIEAFAFEINKSPELKDAKKKLHDLQAKMGLYGFKKVKNEQEIAKTIKRIKWIQNDLNP |
| | VTENVKKRLAQFSLYGSYGRNQDRFMDFATRYLAEQKYFGVDAEFKMYKYFTSEEQNTELATYELPKDK |
| | KAYDKLRFHKGKLVHFSTFENHLKKYESWDTPFVIENNAIQVKLSIRQDNKKEPIEKILSIQRALMLYFLED |
| | ALFQTGNNNIIENKGRILVEQYYTVYNNDFVQSKTVLEENDSISPEQKNALKKIVPKRLLHRYFARSNKL |
| IMG_ | MDTNEAYTAYNSRNSFKRIFDFKGEIAPIAEKANLNYDIKAKNAINREQRLHYFTVGHTFKNIDTEHVFEILL |
| 3300029989_3 | DEETREKRPYTFLSLQQFNTDFCTAIKEVISNIRHINSHYIHDFERIKTDNIPPEIITFLKESFELAVIQIYLKENN |
| SEQ ID NO: | ITYLQFIEQKNTDTTIVKYLHDKFYSLDNTKTDTKNDTSPSLAEYIAFRNTFKTLSKEKALDSLLFVTVDAGF |
| 4554 | PWKLEETHTACTITQGTYLSFNACLFLLSLFLYKSEANQLISKIKGFKKNKTDEEKSKREIFSFFSKKFSSQDI |
| | DSEENHLVKFRDLIQYINHYPVEWNKDLKLESGHPLMTDKLIAKITDMEIDRAYPDYAGNNKFQAYAKEL |
| | LWNVPSKTTFTTEEIEAFAFEINKSPELKDAKKKLHDLQAKMGLYGFKKVKNEQEIAKTIKRIKWIQNDLNP |
| | VTENVKKRLAQFSLYGSYGRNQDRFMDFATRYLAEQKYFGVDAEFKMYKYFTSEEQNTELATYELPKDK |
| | KAYDKLRFHKGKLVHFSTFENHLKKYESWDTPFVIENNAIQVKLSIRQDNKKEPIEKILSIQRALMLYFLED |
| | ALFQTGNNNIIENKGRILVEQYYTVYNNDFVQSKTVLEENDSISPEQKNALKKIVPKRLLHRYFARSNKL |
| IMG_ | MDTNEAYTAYNSRNSFKRIFDFKGEIAPIAEKANLNYDIKAKNAINREQRLHYFTVGHTFKNIDTEHVFEILL |
| 3300029998_6 | DEETREKRPYTFLSLQQFNTDFCTAIKEVISNIRHINSHYIHDFERIKTDNIPPEIITFLKESFELAVIQIYLKENN |
| SEQ ID NO: | ITYLQFIEQKNTDTTIVKYLHDKFYSLDNTKTDTKNDTSPSLAEYIAFRNTFKTLSKEKALDSLLFVTVDAGF |
| 4555 | PWKLEETHTACTITQGTYLSFNACLFLLSLFLYKSEANQLISKIKGFKKNKTDEEKSKREIFSFFSKKFSSQDI |
| | DSEENHLVKFRDLIQYINHYPVEWNKDLKLESGHPLMTDKLIAKITDMEIDRAYPDYAGNNKFQAYAKEL |
| | LWNVPSKTTFTTEEIEAFAFEINKSPELKDAKKKLHDLQAKMGLYGFKKVKNEQEIAKTIKRIKWIQNDLNP |
| | VTENVKKRLAQFSLYGSYGRNQDRFMDFATRYLAEQKYFGVDAEFKMYKYFTSEEQNTELATYELPKDK |
| | KAYDKLRFHKGKLVHFSTFENHLKKYESWDTPFVIENNAIQVKLSIRQDNKKEPIEKILSIQRALMLYFLED |
| | ALFQTGNNNIIENKGRILVEQYYTVYNNDFVQSKTVLEENDSISPEQKNALKKIVPKRLLHRYFARSNKL |
| IMG_ | MDTNEAYTAYNSRNSFKRIFDFKGEIAPIAEKANLNYDIKAKNAINREQRLHYFTVGHTFKNIDTEHVFEILL |
| 3300030339_4 | DEETREKRPYTFLSLQQFNTDFCTAIKEVISNIRHINSHYIHDFERIKTDNIPPEIITFLKESFELAVIQIYLKENN |
| SEQ ID NO: | ITYLQFIEQKNTDTTIVKYLHDKFYSLDNTKTDTKNDTSPSLAEYIAFRNTFKTLSKEKALDSLLFVTVDAGF |
| 4556 | PWKLEETHTACTITQGTYLSFNACLFLLSLFLYKSEANQLISKIKGFKKNKTDEEKSKREIFSFFSKKFSSQDI |
| | DSEENHLVKFRDLIQYINHYPVEWNKDLKLESGHPLMTDKLIAKITDMEIDRAYPDYAGNNKFQAYAKEL |
| | LWNVPSKTTFTTEEIEAFAFEINKSPELKDAKKKLHDLQAKMGLYGFKKVKNEQEIAKTIKRIKWIQNDLNP |
| | VTENVKKRLAQFSLYGSYGRNQDRFMDFATRYLAEQKYFGVDAEFKMYKYFTSEEQNTELATYELPKDK |
| | KAYDKLRFHKGKLVHFSTFENHLKKYESWDTPFVIENNAIQVKLSIRQDNKKEPIEKILSIQRALMLYFLED |
| | ALFQTGNNNIIENKGRILVEQYYTVYNNDFVQSKTVLEENDSISPEQKNALKKIVPKRLLHRYFARSNKL |
| IMG_ | MKSSVENIYYNGVNSFKKIFDSKGAIAAIAEKSCRNFDIKAQNVVNREQRMHYFSVGHTFKQLDTENLFEY |
| 3300030000_3 | VLDEQLRIKTPTRFVSLQHFDKEFIENIKRLISDIRNINSHYIHRFDPLKIDAIPSTIVTFLKESFELAVIQIYLKE |
| SEQ ID NO: | KGINYLQFSENPHADQKLVAFLHDKFLPIDEKKIAMLQNETPQLKEYKEYRKSFKALSKEAAIDQLLFAETE |
| 4557 | TDYDWKLFESHPVFTISAGKYVSFYACLFLLSMFLYKSEASQLISKIKGFKKNTTEEEKSKREIVTFFSKKFN |
| | SMDIDSEEKQLVKFRDLVSYLNHYPVAWNKDLELESSNAAMTDKLKSKIIELEINRSFPSYEGNNRFAIFAK |
| | YQIWGKQHPNKFIQTEYNNAAFSNEEITAYTYETNSCPELKDAHKKLAELKAAKGLFGNRKEKNERNIEKT |
| | QKSIRKLQHEPNPIKDKLIQRIEKNLLTVSYGRNQDRFMDFSARFLAEINYFGQDARFKMYRFYSTDEQNCE |
| | LEKYELPKDKKEYDSLKFHQGKLVHFSSYKEHLKRYETWDDAFVIENNAIQLKLLFDGVENTITIQRALLIY |
| | LLEDALRNSQNNTAENAGKELLEAYYSHNKVDFSAFKHILTQQESIEPQQKTEFKKLLPRRLLNHYSPAIGN |
| | CQTAPSSLPLLLEKAILAEKRYSSLTAKAKAEGNYDDFIRRNKGKQYKLQFIRKAWH |
| IMG_ | MDNSTSKSFKRFFEFKGNVAPIAEKANRNFNIKNLNPINTQQRLHYFAIGHVFKSIDTEKIFTVLLDEVAKVK |
| 3300001881 | KPTKFSALQNTEFTFINELKCLMSDIRNINSHFIHDFEKIKIDSIDKNIIEFLKQSFELAVLQTCMDEKNINYEE |
| SEQ ID NO: | FTGGGNPEKEIVDFLCDKFYPKIDNSKDLSEHQKLISDFKKKSKDEAINEILFINVSSDYNWNIFETHTVFKIS |
| 4558 | KGKYLSFEACLFLLAMFLYKGEANQLISKIKGFKRNDDNKFMSKRNLFTFFSKKFSSQDIDSEENHLVKFRD |
| | LVQYLNHYPTVWNKYLELGSNNPIMTERLKEKIIELEIKRCFPELASNSSFNQFAINYIFKNGKIIECENNNIY |
| | LDIINKNDEVRKIYFSIKNNEFNRSEFKDNSFKMFALKYVVKEYYKENKAYADYLTKQIKDKEKTFDEELIT |
| | NKKVEKLKKQISQNLNFIFYGRNQDRFMEFATRYLAETGYFGKDAKFKMYEFFTTDEQIEEIDRLKRTISKK |
| | EFDKLKFHQGKLVHCSTYADHIAKYKNWDTPFVVENNAVQLTVCFDNGQRKILSIQRNLMPYFLEDALYN |
| | MQNDKIEGAGKILIENYYNYHKEGFEKSRLTLKQNDTISLLEKATFKKILPKRLLHRYSPAVQNNLPENSTY |
| | KQILTKTKEAEERYV |
| IMG_ | MNTAKKFHRFFEVKGNVAPIAEKADKNFILKEKNNVNLQERLWYFAIGHVFKQLDTKAIFDYKVNETTRE |
| 3300033446 | SKPQKFTSLTSDNFSFLKKIKSFIGNIRNINSHYIHDFSVIKLNTNIEDANNDNSFMMFLKEAFELALIHIYSEE |
| SEQ ID NO: | KGLKYSQFIDDKTNDKKLVEYIRDKFYSLNDSRKNLTSEEKKASEEYKKFRTDFLNKTKSQAINDILFIDNE |
| 4559 | ADFDWTLYETHKVFTIKEGKYLSFDACLFLLTMFLYKNEANELISKIKGFKRSDDNTFRSKRNLFSFYSKKF |
| | SSQDIDSEEGNLIKFRDIVQYLNLYPKHWNSELEFDAKIPQMTKPLKDKIVEMEIERCFP |
| IMG_ | MGETSKDSSNNDFSSSAFYRHFENAGIMGPICEKAVKNFELKGAFFKSSNESSTDLVNRRQRIHYFAIGHAF |
| 3300020385 | KQIDTKTIFEYKIDETAREERPTKYLSLQTNNFSLDKELFNLLRDIRNLNNHYVHIFDKIKVTKLKETNVIAFL |
| SEQ ID NO: | KESFELALIKIYFKEKGHLPNNDNDIVSFLKRIFFPKKTDNKTDSIEQKERNKIWNDFTYSLTSKAQTIDAILFI |
| 4560 | DVENEFDWNINNEVKVLSIKKGKYLSFEACLFLVSMFLYKNEANHLIPKIRGYKRNDDTQMRSKRELFSFF |
| | SKKFTSQDVDAEESHLVKFRDIIQFLNHYPTTWNNDLKLESESKNQKMIKVLKDSIITMEIYRTYPNYNNDI |
| | NFVSFAKDYLFKNKSNELNEEYKNKKLTKAQCEYYEEITQNPHIKIFKNEIANAIKPIAYNLKENAFKIYVK |
| | QYVLKTFFPNKRGYEKFATHRFKKNKRYITEDVEKGFKSQLFSNPKTERLKKRILEDSLIMSYGRNQDRFM |
| | DFSIRYLAEKNYFGADAQFKCYQFYTTFEQENYLNNFKKTHTKKEIDNLKYHNGKLVHFTTYQSHKRNYP |
| | EWDMPFVNQNNSVSIKIILEEKTTDEKNEVAIEKIITIQRNLITYFLEDALYNTDYDGKQLLT |
| IMG_ | MEQKQLESRFNQIFNNKGHTGPIAEKAVKNFETIRQHKVSPRERLHYFAVGHALRNIDKDLKESIFEYNLDE |
| 3300020592 | EQKKQKPTQFTTLQSDFFRFENALLTLLKDIRNCNGHYVHTFDKLQLDEILKLQEKNKEHGILNKDAGCQII |
| SEQ ID NO: | EFLKEAFEFSILIQFLKEKPKEYEKFKKRKNENKNQSLRNLIGGYEKKLVKYLCDKFFPNEEKQKEIRDKFIE |
| 4561 | HNLEEAIEDLLFIPVDEDIEWKLGEEHVVFVIKKGKYLSFYAQLFLLSMFLYKQEANQLISKIRGFKRSEDEF |
| | QYKRNIFTFFSKKVSSQDIHSEEKHLIYFRDIIQYLNRFPTAWNEYLSPERKNLPMTKLLEKYILEEEIFRTFST |
| | YKNDCNRELFLKYTIKRLFCKKAELFDAEKISIDDNLRKKFNYEIDTSPELKNIHEKLKGKLKPKDYYKNIK |
| | RKEELEKEENPEKLKLTKKVTEEKLFTAYGRNRDRFMDFAVRYLAEQNYFGKDAEFKMYMFETTNEQEN |
| | YLKEQKNTADKKVIDQNKYHQGRLTCFKTYQKHKDDYQNWDDPFVFQNNAFQIILTFSNGERKKFSIQRK |
| | LLIYLLEDALFNHSDSIEDKGKQLLEDYFFNTLMPDFEDAKESYKTSDDVNWKHRKLLPKRLIYTVHPPRRT |
| | DSEEQIHPFEKILRETQEQERRYRLLLGKAKNMKLKEEFIKRNKGKHFKLRFIRKAWHLMYFREIYERRAKE |
| | HSHHKSFHITRDEINDFSRWMYAFDEVPPYKVYLRNMLQRKKFMENEEFAELFEKGKSLDDFYRITKKEFS |
| | KRIKNNLFQLKVDSERQYAEILSKKLVYINLSHFIKYLNAKGKLTVENGIIQYKASTNKKYLIDEYYYTEVL |
| | PREEYKVHKHLFNKLRATKL |
| IMG_ | MEQKQLESRFNQIFNNKGHTGPIAEKAVKNFETIRQHKVSPRERLHYFAVGHALRNIDKDLKESIFEYNLDE |
| 3300005281 | EQKKQKPTQFTTLQSDFFRFENALLTLLKDIRNCNGHYVHTFDKLQLDEILKLQEKNKEHGILNKDAGCQII |
| SEQ ID NO: | EFLKEAFEFSILIQFLKEKPKEYEKFKKRKNENKNQSLRNLIGGYEKKLVKYLCDKFFPNEEKQKEIRDKFIE |
| 4562 | HNLEEAIEDLLFIPVDEDIEWKLGEEHVVFVIKKGKYLSFYAQLFLLSMFLYKQEANQLISKIRGFKRSEDEF |
| | QYKRNIFTFFSKKVSSQDIHSEEKHLIYFRDIIQYLNRFPTAWNEYLSPERKNLPMTKLLEKYILEEEIFRTFST |
| | YKNDCNRELFLKYTIKRLFRKKAELFDAEKISIDDNLRKKFNYEIDTSPELKNIHEKLKGKLKPKDYYKNIK |
| | RKEELEKEDESPKN |
| IMG_ | MDFAVRYLAEQNYFGKDAEFKMYMFETTNEQENYLKEQKNTADKKVIDQNKYHQGRLTCFKTYQKHKD |
| 3300005281_2 | DYQNWDDPFVFQNNAFQIVLTFSNGERKKFSIQRKLLIYLLEDALFNHSDSIEDKGKQLLEDYFFNTLMPDF |
| SEQ ID NO: | EEAKGSYKTSDDVNWKHRKLLPKRLIYTVHPPRRTDSEEQIHPFEKILRETQEQERRYRLLLGKAKNMKLK |
| 4563 | EEFIKRNKGKHFKLRFIRKAWHLMYFREIYERRAKEHSHHKSFHITRDEINDFSRWMYAFDEVPPYKVYLR |
| | NMLQRKKFMENEEFAELFEKGKSLDEFYHLTKQEFSKRIKNNLFQLKVDSERQYAEVLSKKLVYINLSHFI |
| | KYLKCEREN |
| IMG_ | MSTRYTNQKKVEENINYVFSNKGIFAAVAEKTLTNYKSKFNEGQTIQPNLHYFAVGHTFKNIDTKRIFDYQ |
| 3300031208_2 | LSEDEMDILPTKYFSLQQNKFSFPIDGQTKTDKLYLLLDNIRNINAHFIHDFNFLKVDNIDENIICFLIDSFELA |
| SEQ ID NO: | LIKGVFAKKYGNKKHELGLDFLSNDLKDEILEEILENLDDELIVFMKEIFYQTLYVVDKKEWRNKELNQEK |
| 4564 | KDFLDSHLSSKEDWINWILFNCVEEDIDWFLNNYGDSDPHPDSKNHKHKVLTIEKGKHLSFEGSLFMMTMF |
| | LYANEANYLIPKLKGYKKNGTPQDASKLEVFRFFAKKFKSQDVDSEHKQYVKFRDMIQYVGKYPTVWNK |
| | HIHLDNYYVNELKVSILENEITQLFEQDIQNYFETKYGLQKFAKAKGDMHYAFVEVAKSYLQKKTISYKGK |
| | YQDDSIAILETLAEQITNKRQLKNIEVKLLRFSDTDYQSLSSIEKTKKQRLDKSKKMFLDKIKESKKTINKTT |
| | EKFAKRVEDNLLYISNGRNSDRFMVFACRFLAEINYFGKDAQFKMYENYYSEEEQNALKDKKQNLTQKEF |
| | DKLHYHGGKLTHFETFENHTKKYPNWDMPFVVQNNAIYVKIPGINFIKNTAFCIQRNVINYLLEHALGENY |
| | KDQQGRKWLFDYYEHKTEATDNAKRILTESKPIEASSKTKLKKLLPKKLFPKYSASETEKNILRKYLEQAEE |
| | SEKLYENQKDKAKAENRLDLFVNK |
| IMG_ | MSTRYTNQKKVEENINYVFSNKGIFAAVAEKTLTNYKSKFNEGQTIQPNLHYFAVGHTFKNIDTKRIFDYQ |
| 3300031613 | LSEDEMDILPTKYFSLQQNKFSFPIDGQTKTDKLYLLLDNIRNINAHFIHDFNFLKVDNIDENIICFLIDSFELA |
| SEQ ID NO: | LIKGVFAKKYGNKKHELGLDFLSNDLKDEILEEILENLDDELIVFMKEIFYQTLYVVDKKEWRNKELNQEK |
| 4565 | KDFLDSHLSSKEDWINWILFNCVEEDIDWFLNNYGDSDPHPDSKNHKHKVLTIEKGKHLSFEGSLFMMTMF |
| | LYANEANYLIPKLKGYKKNGTPQDASKLEVFRFFAKKFKSQDVDSEHKQYVKFRDMIQYVGKYPTVWNK |
| | HIHLDNYYVNELKVSILENEITQLFEQDIQNYFETKYGLQKFAKAKGDMHYAFVEVAKSYLQKKTISYKGK |
| | YQDDSIAILETLAEQITNKRQLKNIEVKLLRFSDTDYQSLSSIEKTKKQRLDKSKKMFLDKIKESKKTINKTT |
| | EKFAKRVEDNLLYISNGRNSDRFMVFACRFLAEINYFGKDAQFKMYENYYSEEEQNA |
| IMG_ | MSTRYTNQKKVEENINYVFSNKGIFAAVAEKTLTNYKSKFNEGQTIQPNLHYFAVGHTFKNIDTKRIFDYQ |
| 3300028408 | LSEDEMDILPTKYFSLQQNKFSFPIDGQTKTDKLYLLLDNIRNINAHFIHDFNFLKVDNIDENIICFLIDSFELA |
| SEQ ID NO: | LIKGVFAKKYGDKKSKLRLDFLSNEARDEILTDILENLDKELIVFMKEIFYQTLYVVDKKEWRNKELNQEK |
| 4566 | KDFLDNHLSSKEDWINWILFNCVEEDIDWYLNNYGDYDPHPDSKNHKHKVLTIEKGKYLSFEGSLFMMTM |
| | FLYANEANYLIPKLKGYKKNGTPQDASKLEVFRFFAKKFKSQDVDSEHKQYVKFRDMIQYVGKYPTVWN |
| | KHIHLDSYYVKDLKATILENEIIQLYQEDIKNHFQSICGLKQFEDSQEEIQLAFIEVAKDYLQNKEIYYTGEY |
| | REDCITILKTSAEHLANKRQLKDIQKQLKKYINKAYKSLSSEDQGKKKKLDISKLKYIDKIRKSKNSINTTTD |
| | KFAKRVEDNLLFISNGRNSDRFMVFACRFLAEINYFGKDAQFKMYENYYSEEEQNALKDKKQNLTQKEFD |
| | KLHYHGGKLTHFETFENHTKKYPNWDMPFVVQNNAIYVKIPGINFIKNTAFCIQRNVINYLLEHALGENYK |
| | DQQGRKWLFDYYEHKTEATDNAKRILTESKPIEASSKTKLKKLLPKKLFPKYSASETEKNGSSLGLR |
| IMG_ | MKKNINPFNLFFLEKGYIGSIAQKAENNFSTNCKALNGSQISDKENKATSRIYYFAVGHAFKNIDTKAIFAY |
| 3300028650 | KYDDDKKNLRATQYVHIQRNVFDQGLRDFIGEKVHAIRNMCSHYVHLFNDIKLEDEDSDKANIIQDQFIPF |
| SEQ ID NO: | VKEAFKLAVVQAYPGESDMTIDKVDDAKIIRFLYDNFISKGEYDDLEEKKQFFKLSSDKALEKVLFIDLDED |
| 4567 | QSICIGPGYEVFTATKGRYLSFYASLFLLSMFLYRNEAEILISRIKGFKDKRDIGNIAKRSVFTFFSKKVSSRD |
| | VDNEEMPLVKFRDIMQYLNHYPTAWNDQLSDYSSSESSKNLCNKIVEMDIKRLFHEMFETENEESLRFLTY |
| | VKLTQYEALYPKKYVRAEFLKADFTSRETEHFDIIIKESKELKGCRKKLQDLLHADSLGNEKEKEIAECTEKI |
| | QELEGAETNPDLDAVRKQIDDHLFITSYGRNQDRFMDFAARFLAEVEYFGKDAFFKVYRFETIEEQSKYLE |
| | KVKNQLPKEQYDKLKMHRGRLATYMNHNQIINYYSSFDIPFVTENNSIQVFMPSSKIIEDMDDQMLKKIFKG |
| | KGTFYVIQRPLIIYLLEQFLYEPSGRKKMTFKALIENYCGRRKKGLEKYETLLLNDSDSKAAELQDQ |
| GCA_ | MNITDFIKKKTDPFIRNKGLLAPLSEIAYRNFELVCDNNEKSGDLSLQAISSIYQFSIGQTFKSHNIKQLFNYQ |
| 000212915.1_ | LNDEKDRFVPTKYLSLQKKQFIDNEIASTLLKLVSAIRELNQNYTHNLEPLRIGNNIITPQIIEFLHDLFEVSII |
| ASM21291v1_ | MLLNKSVKDRNNFTSQYNEDSLNLLLKEFILTLFFPETSYTTEEIEVLRKKSKDELINEVLFFDVQSVYQWK |
| genomic_2 | VVNNPLMMPIQCGKYLSFTSCLFINSLFLFKEDTKIIFPNFPFLKNKTDETQVLQMFFSLFANPYTLHYIPSQH |
| SEQ ID NO: | LRIAKHKDIIEYLNLYPSPWQEALNSQSPCFPMSHLLKDFLIERECNRVFLKVPHLNLILIPIIINRLMVTKIIA |
| 4568 | |
| IMG_ | LIDEYYYTEVLPREEYKVHKHLFNKLRATKLEDALLYELAMKYLREDNDIVEKAKSKVSDIQSSEISFDIKD |
| 3300020592_2 | YYGNHLYTLIVPFNKLETLSILIRYKTKQEKNSKLQRTSFLGNIYHLLAFLDKNYKQLSKYKDDGGFKKIVK |
| SEQ ID NO: | NFKNKKRLSFVELNTINGYIISGAVKFSKVHMELERYFINKHKIKANHIYIDLEDIKDDSNKQVFGNYYDSK |
| 4569 | LRIRNKAFHFGVPTNFFFNIEIEKIEKKFILEEVKLQNVSSFDKLNQNAKSVCRVFMEVLHSDLYRRDKNKS |
| | KEELRREFEEKYFNEIITTV |
| IMG_ | MRKGKLTVENGIIQYKASTNKKYLIDEYYYTEVLPREEYKVHKHLFNKLRAAKLEDALLYELAMKYLKED |
| 3300005281_3 | EEIVEKAKSKVSDIQSSEISFDIKDYYGNHLYTLIVPFNKLETLSILIRYKTGQENELKNKKTSFLGNIYHLLAF |
| SEQ ID NO: | LDKNYKQLSKYKDDGGFKKIVKNFKNKKRLSFVELNTINGYIISGAVKFSKVHMELERYFINKHKIKANHI |
| 4570 | YIDLEDIKDDSNKQVFGNYYDSKLRIRNKAFHFGVPTNFFFNIEIEKIEKKFILEEVKLQNVSSFDKLNQNAK |
| | SVCRVFMEVLHSDLYRRDKNKSKEELRREFEEKYFNEIITTV |
| GCA_ | MIQGVKFSENKERFADWFVHYKDYEHYQKFYDTNLYPVESIEDKERQKLEATIKKQQKNDVFTLLMIKKIF |
| 900618225.1_ | NDLFNQDFEANLYEMYQSKEERENNQLIAKETQNRNLNFIWNKPIAIDLFDGKVKIDEVKLKDVGSFRKYE |
| NCTC13469_ | NDKRVQTFIKYHPEIQWIPYLPNTWEGINLPVNVTERQIDRYEKVRSEELLKEVQAIEKYIYEQVNDKTELLQ |
| genomic | NGNQNFKNYLVNGLLKQIQGIDVSNFKFINQQKFETINVKDLDNEASALEQKVYILINIRNQFSHNQFPKSTF |
| SEQ ID NO: | YQFCQKILLIEEDELFADYYLRLFKLLKNELLD |
| 4571 | |
| IMG_ | MTETTKVALPNDKNQAINKLFDSADRQKTLAKIEKELPFFQYYLNQAVVNLQKLGAPDLSGDEDKAQKLI |
| 2061766007 | EELPDAKIQILADFLWLFKTDNPGEDFKDYRKITTMLVDKIFRLRNFFCHTERGDIKPLLTNAAFYHFFAGW |
| SEQ ID NO: | ALGEARLHSLEGGVKSDRIFKMSIMNAQEINKDDRTRNIYAFTRRGIIMLICMALYKDEAIEFCQALDDMKL |
| 4572 | PRVELDEELEQSDSEQTELRKKAGIRKAYHLVFLYFSKKRSFNAVDEENHDFVCFTDIIGYLNKVPMVSMD |
| | YLALNEERKRLAELEAASTESDENKRFKYTLHRRMKDRFLSFITAYCEDFNLLPSIRFKRLDISPSIGRKRYC |
| | FGIESDNSVRQSRHYAIEKDAIRFEWRAKQHYGDIHIDSLRSAISASEFKRLLLASRSTRTGKNFNASNELDA |
| | YFTAYHKVLEKMLNEPECDFINREGYLPELTAITGASREELMDNPTLLEKMRPFFPENITRFFIPRDNIPDNQ |
| | TLLEQLKNALQNAIKHDDDFIARMDGATEWTSKYADVPPEKRPKRPQEYRFNNNAFISKVFALLNLYLPDD |
| | RKFRQLPKGKQHRACMDFEYQTLHAIIGRFASDPQELWDYLKGIKTVYRYEGKKRIPDHTINVIDSKRXXX |
| | XXXLQKKRTSSTRKRNASTGIPSLMQMDDSHATPSRCLHAPLSSCIRNSAKSFSHNIKVHNWTGFAHSFRK |
| | TAASSAYALDSPSLMTPSSKPSSTLTRPSGSMPSMKRKTALGKTARS |
| IMG_ | MNAQEINEDDKTKNTYAFTRKGIVLLACMALYKDEATEFCQSLQDMKLPTVELEEDESIDDAEKATLRKK |
| 2061766007_2 | ASIRKAYHLVMSYFSQKRSYNAIDQENHDFVSFTDIIGYLNKVPTVSMDYLALNEERRKLAELDAKSTESEE |
| SEQ ID NO: | NRRFKYTLHRRAKDRFLSFAAGYCEDFNILPCIHFKRLDISDHIGRKRYIYGMENDNSVRQSRHYAIDKDAI |
| 4573 | RFEYRPSGHYGDIHIDYLRSAISAKEFKRLLLATRSTRTSIFNPSEALDAYFSAYHKVLEKMLNEPDCDFIDR |
| | TGYXXXXXXXXXXXXSHPG |
| OLZV01.1 | MKKQNKNNHNRSCKGRFGEKNISQNCKRNIYLPNDLKRALYKLKIDQPGYSEQKNFFVYLSFATNNIFEIA |
| SEQ ID NO: | GISHDFSTDGIKVWDEIKRLKMVDKLARFLWLFRIEDPAKECPDYEEITEGIVKKLLELRNLFAHINNKKSIE |
| 4574 | AFLLDNKLANALQWGLMDVARENVLKPGLSTAKLFKQRLVTPHNDTKYEFTRKGIIFLICLALFKDEAFHF |
| | CSSLNDLKDMRKDAEWQRLRNDDAAEELKKYMTRNNYKNPSQTRAQVDMLTYFSMRSSYKAILGIGSDD |
| | QGSSAIDKEERDYKIFADIIGYLNKVPVECYDYLELADERRMLKDLNDKSEESEENKEYKYDLKSNRRLKN |
| | RFLPLAIGYCEDFDLFPSIKFKRLDISEQIGRKRYCYGKENGNANGMDRHYAIHDGSVGFEYCPDNHYGDL |
| | RISSMRSSISTYELKRLLLLETVFRCDKKKIDEAISNYFSAYHRVMERMLNASYSGDFELEDFREDFSLVSGL |
| | EPEEISKDKLFEQMGLYFPDSLLRFFLNKDNNPTPKELKALLKKKIAYRQRQCEDFLNKIDEVYKRRTSTKE |
| | ELSSAGKPVKISDGVLIRKVFNLLNIFLKPEEKFRQLPKSEWHKGNKDFEYQTLHAIIGKFPLDKNKRFWSFI |
| | LECRPGLKDIIGKLQAKYNSEYERRGASEARRGLNAL |
| OLZZ01.1 | MKKQNKNNHNRSCKGRFGEKNISQNCKRNIYLPNDLKRALYKLKIDQPGYSEQKNFFVYLSFATNNIFEIA |
| SEQ ID NO: | GISHDFSTDGIKVWDEIKRLKMVDKLARFLWLFRIEDPAKECPDYEEITEGIVKKLLELRNLFAHINNKKSIE |
| 4575 | AFLLDNKLANALQWGLMDVARENVLKPGLSTAKLFKQRLVTPHNDTKYEFTRKGIIFLICLALFKDEAFHF |
| | CSSLNDLKDMRKDAEWQRLRNDDAAEELKKYMTRNNYKNPSQTRAQVDMLTYFSMRSSYKAILGIGSDD |
| | QGSSAIDKEERDYKIFADIIGYLNKVPVECYDYLELADERRMLKDLNDKSEESEENKEYKYDLKSNRRLKN |
| | RFLPLAIGYCEDFDLFPSIKFKRLDISEQIGRKRYCYGKENGNANGMDRHYAIHDGSVGFEYCPDNHYGDL |
| | RISSMRSSISTYELKRLLLLETVFRCDKKKIDEAISNYFSAYHRVMERMLNASYSGDFELEDFREDFSLVSGL |
| | EPEEISKDKLFEQMGLYFPDSLLRFFLNKDNNPTPKELKALLKKKIAYRQRQCEDFLNKIDEVYKR |
| IMG_ | MSYNITVGSRQNGRASGFHGGAPKKRTYLSGDFARDMRELRIKGTIRSPQTRKETYVDETPQFITYLTLALQ |
| 3300031998 | NISDIIGEDVTKMRSKNSVERSLSRSGKLWEVASFLWLHAEDQPKRDFAKLSKEAAAKGEDPDYAKFAGAI |
| SEQ ID NO: | VVKLWELRNMFVHWSQSRSAGVLVVNREFYRFVEGELYSAAMPDAIGSGRKSEKMFKLRLFNPHDDAKL |
| 4576 | QYEFTRKGMIFLVCLALYRHDASEFIQQFPDLQLPPREWEMEKGYKKRMTEEDLVSLRKKGGSIKAILDAF |
| | THYSMRASRTDIDLKNKEYLNFANVLTYLNKVPMASYNYLTLREEAQALAEAAEKSTESEENKRFKYLLH |
| | PRQKDRFLTLALAFIEDFHVLDCIRFKRLDITVRPERSRYMFGPIEAGTKNEFGYELSDANGMDRHYVISHG |
| | NAEFEYVPEKSDHENRSIRISRLRGRVGEGEVMRLLLAFFTTRDANVPAEKNPVNTELHAYLRSYHRILER |
| | MLNAKTLDGLKFDSPDFKNDFKRVSGKSVDSLTKENFVEEMKPFFPAGITRYFVGDEMKLDTRALQDILAS |
| | KLAARADRASDFLKRLDRLTDWRELDEEARKRVGPPICKIGELKYPPRTCKMTDAQLIKRVLDYINLNLND |
| | PNDKFRQLPRGLRHRGIRDVEFQMLHRDIGRFGSNPDGLWRTLEKREALNGED |
| IMG_ | MKAKLPMNHQDALCHLEIEGCVRGSNVHLESAFLLYLNQAVVNIQERTGIGDRYFDPDTVWSEIRKKGPG |
| 3300000505 | VVERLASFLWLFREEDPERDWGKDYEEYTEKIVKRIFQLRNWFAHRDRMAGKDSLIVDRAFYVLIEGLLG |
| SEQ ID NO: | AAAREAADGPGMKMAKVWKAKLLSLQDKNAVDKALETYYLTKRGLIFLICLALYKDDATEFCQLIPELRL |
| 4577 | EDRYEEALEGYEVPDPKKKGSAKAMRAFFTYYSMRKGRQDLDAGDLDRMCFSDILTDLNKVPLAAHDYL |
| | PLAEERQDLDARREVSTESEANKRFKYELHPRMKDRFLSTAAGYIEDFDVLPSV |
| IMG_ | MMKQAKQVLLPADPKEALLKLWDRPDKSERRWLFEHELPYFQFYLNLAITHIQGIAHLDESKFDEEAIVKQ |
| 2061766007_4 | IMKLDKETRFRLADFLWLSKVDDAKSLFYKDCCPQECAISFPEEKKPCNDEAGNLAEGKDDVKSFFPCCNE |
| SEQ ID NO: | RCPLRAKDECSNYDARIIVQLYRLRNFLAHYTRPDTTIGALLTDYQFYTFFAGWLFGEAKSKALNGQIKTD |
| 4578 | KLHKMKLMTQQTEGKDTPREQCQYAFTRKGLVFLICLALYKHEAHEFCQALVDMKLPTKELLAVEQPDE |
| | DAQTALRKKKSQREASRELFTYFSMRESYGAVWKDDHNFIYFTDLIEYLNTVPLVSLDYLALRKERELLAE |
| | DCAKSEESESNKLWKYSLHGRQKERFLSFLTAYCEDFDIIPSIEFKRQDLRPSIERHRYCFGEDEKRKNFTSD |
| | SADSDISRDRQDRHYAISRDCVHXXXXXXXXXAYSYCGVAQCD |
| mgm4547164.3_ | MKDIVSYFRELLSGTKYALADDATEEKISELIYNVGYSKSAKDLPNKNLKKLTQLQIVQTGIETLMRSKGK |
| 8 | KPEDLPENIHAFVFNNAEAIQASDPGLTTDEANPVTARFLQMLMGDGDQNEGRLERRLQWVDGQLAKFSR |
| SEQ ID NO: | SSSAQFAKDNRYATKGYKDVRYGRLAEILAESMLLWQPTKDTDDSIERGRNKLTGLNYRRLVDFLAIYSE |
| 4579 | DSTAKKERDGIVEEYSGLEALECVLNEAKLIGSVTYHPFLSAVLKKAPRNIENLYLAYMNAEKNYLINLKK |
| | TFAKKAATDKIEDLQSQAPAFVHPFRERWTDKVQVADNVRRMAARYLESGSTLLLPDGLFTDAILSQLQR |
| | RGLLQDVFAEAANEQDETEKELLEQRNRNVSFLISRYMESMGDHCQTFYDGDTPIFYRGYDLFKKLYGKK |
| | VRNEQLPFYMSRDVIAAELKAKEELVKKIENYCVQQNKAEAEEGMIRDINHIKKNERAILRYKVQDMVLFL |
| | TAKKMLQSQQTLQDGNATQNQESHRVNLGRQTQAAYVRQQQTLLNRSERIERMSLQDIFDGDALNETMD |
| | YEYRIDVTWKLRDEQGRVLKFKADGQPIGFDEDGNLLEKGGKPKVFKRKVFVTQKNVAIKNFGRIFRIVRD |
| | ERLEKLLILLIMKVEGANLQETGQDYTVSVAELANEFTTFDSLRTDAFELIHELEKTAYPCLTNKESNETFQF |
| | KNMMRLICDTEEEAVAINQYRISFAHSLYGIEETSIGDGLQIPLVSTRMKEQMAARSDQIKEHLAAQN |
| IMG_ | MMRFQPAKRSADNMGVPGSKANSTEYRLLQEALAFYSTYKDRLEPYFRQVNLIGGTNPHPFLHRVDWKK |
| 3300014026_2 | CNHLLSFYHDYLEAKEQYLSHLSPADWQKHQYFLLLKVRKDIQNEKKEWKKSLVAGWKNGFNLPRGLFT |
| SEQ ID NO: | ESIKTWFSTHADKVQIADPKLFENRVGLIAKLIPLYYNKVYEDKPQPFYQYPFNINDRYKPEDADKQFTAAS |
| 4580 | SKLWNEKKARYKNAQLEQLKKKKDLKYLDFLSWKKLERELRMLRNQDMMVWLMCKDLFAQCTVEGVE |
| | FADLKLSQLEVDVTVQDNLNVLNNVSSMILPLSVYPSDAQGNILRNSKPLYTVYVQENNTKLLKQGNFKSL |
| | LKDRRLNGLFSFIAAEGEDLQQHPLTKNRLEYELSIYQTMRISVFEQTLQLEKAILTRNETLCGNNFNNLLNS |
| | WSEHRTDKKTLQPDIDFLIAVRNAFSHNQYPMSTNMVMQDIEKFXXXXQTPKLAEKDGLGIASQLAKKTK |
| | DAASRLQNIINGGTN |
| IMG_ | MEGTKMRRLGVSVYPGHSEIEDILDYLRLAATYGFSRVFTCLLSVGNQEKTIADFKEAVKLATSLGMEVIA |
| 3300008679 | DVDLSVFEKLDLKYDNLEFFKDMGLTGIRLDGGFSGQEEAGMTFNPQGLMIELNMSIENRYLENIAAFQPK |
| SEQ ID NO: | FEKLIGCHNFYPHRYTGLSVQHFLNTSKRYKDLGLRTAAFVNSKVATTGPWPVEEGLCTLEMHREWEITSA |
| 4581 | AKWLWATELIDDVIVANSFASEEELKEYLKGKDIEIVHLPKQMIAILESKPKDMVKEAKRKQKEMVKDTK |
| | KLLAALEKQTQGEIEDGGRNIRLLKSGEIARWLVNDMMRFQPVQKDNEGNPLNNSKANSTEYQMLQRSLA |
| | LYNKEEKPTRYFRQVNLINSSNPHPFLKWTKWEECNNILSFYRSYLTKKIEFLNKLKPEEWKKNQYFLKLK |
| | EPKTNRETLVQGWKNGFNLPRGIFTEPIKEWFKRHQNDSEEYKKVEALDRVGLVAKVIPLFFKEEYFKEDA |
| | QKEINNCVQPFYSFPYNVGNIHKPEEKNFLHCEERRKLWDKKKDKFKDYKAKEKSKKMTDKEKEEHRSYL |
| | EFQSWNKFERELRLVRNQDIVTWLLCTELIDKLKIDELNIEELQKLRLKDIDTDTAKQEKNNILNRIMPMQL |
| | PVTVYEIDDSHNIVKDKPLHTVYIEETKTKLLKQGNFKALVKDRRLNGLFSFVKTSSEAESKSKPISKLRVE |
| | YELGAYQKARIDIIKDMLALEKTLIDNDKNLPTNKFSDMLNSWLEGKGEANKVRFQNDVDLLVAVRNAFS |
| | HNQYPMYNSEVFKGMKLLSLSSDIPEKEGLGIAKQLKDKIKETIERIIEIEKEIRN |
| IMG_ | MIKDTKKRLATLDKQVKGEIEDGGRNIRLLKSGEIARWLVNDMMRFQPVQKDNEGKPLNNSKANSTEYQ |
| 3300008304 | MLQRSLALYNKEEKPTRYFRQVNLIKSSNPHPFLEDTKWEECYNILSFYRNYLKAKIKFLNKLKPEDWKKN |
| SEQ ID NO: | QYFLMLKEPKTNRKTLVQGWKNGFNLPRGIFTEPIREWFKRHQNNSEEYEKVEALDRVGLVTKVIPLFFKE |
| 4582 | EYFKEDAQKEINNCVQPFYSFPYNVGNIHKPDEKDFLPSEERKKLWGDKKDKFKGYKAKVKSKKLTDKEK |
| | EEYRSYLEFQSWNKFERELRLVRNQDIVTWLLCTELIDKLKVEGLNVEELQKLRLKDIDTDTAKQEKNNIL |
| | NRIMPMQLPVTVYEIDDSHKIVKDRPLHTVYIEETKTKLLKQGNFKALVKDRRLNGLFSFVDTSSEAELKD |
| | KPISKSVVEYELGEYQNARIETIKDMLLLEETLIKKYKTLPTNKFKKMLKGWLEGKDEADKARFQNDVKLL |
| | VAVRNAFSHNQYPMRNRIAFANINPFSLSSANTSEEKGLGIANQLKDKTKETIEKIIKIEKPIETKE |
| IMG_ | MKAIIYARVSTEMQEEGRSLEFQIRKCEDFCKMSGYKLKEVIQDVESGGNDNREGFLKLQQEIKKKSFDVL |
| 3300009381 | VVYESSRISRITLTMLNFVLELQKSNIKFVSISQSEINTTTPTGMLFFQIFAVLADYERKQISMRVKSNKWAR |
| SEQ ID NO: | AKAGIWQGGNIPIGYKKDEHNNIVIDPETSEDVINIFNTYLNTKSISETASIFNRNISSIKWILQNEFYIGNLMY |
| 4583 | GRKENNINTGEVKINKEITIFKGNHQALISEDLFREVQRQMLFKQRVIRKEGKFLFTGILECICGGKMFKNGV |
| | NYRCDKCKKAISMNKAEKFIIHKLLNLKELEFLNELQPQNWASDNYFLLLRAPKNDRQKLAEGWKNGFNL |
| | PRGLFTEKIKTWFNEHKTIVDISDCDIFKNRVGDVA |
| IMG_ | LKSNILPENDDDYDADLHHPFLLNVLDEEPSSVEEFYEIYLEEELNHIDYMITFLKKHKAKGTALYIPFLHAN |
| 3300028805_2 | RSRWKNADEDTMKKLATRYMEQPLQLPNGMFTESIFKLLMEIDNPDLHEELVKAENPDADKNLANNVSY |
| SEQ ID NO: | LMSIYFKHVERDHSQPFYNTTAIEGEPSPYRHVYRIFKKLYGQQIPHTNQTTTPAYTVEEINGLRKQALTDIA |
| 4584 | KYVEKDISNWKDRQQFKFEQKLKKKLKKENDRRYKNHEQQLNVYEEVNKVVQQEINTMRTTTTAKLIRQ |
| | LKKVYDNERTIRRFKTQDMLMLIMAREILKAKSQNKDFTKDFCLKYVMTDSLLDKPIDFDWSVNIEKKKK |
| | NEEGKIEKEIIRKTIRQEGMKMKNYGQFYKFASDHQRLESLLSRLPDELFLRAEIENELSYYDTNRSEVFRLV |
| | YIIESEAYKLKPELANDANTDKEWFYYADKKGKKHPKRNNFLSLLEILAAGKDGILNEDEKRSLQSTRNAF |
| | GHNTYDVDLPTVFEGKKEKMKIPEVANGIKDKIENQTEELKKSLQK |
| IMG_ | LKSNILPENDDDYDADLHHPFLLNVLDEEPSSVEEFYEIYLEEELNHIDYMITFLKKHKAKGTALYIPFLHAN |
| 3300031994_2 | RSRWKNADEDTMKKLATRYMEQPLQLPNGMFTESIFKLLMEIDNPDLHEELVKAENPDADKNLANNVSY |
| SEQ ID NO: | LMSIYFKHVERDHSQPFYNTTAIEGEPSPYRHVYRIFKKLYGQQIPHTNQTTTPAYTVEEINGLRKQALTDIA |
| 4585 | KYVEKDISNWKDRQQFKFEQKLKKKLKKENDRRYKNHEQQLNVYEEVNKVVQQEINTMRTTTTAKLIRQ |
| | LKKVYDNERTIRRFKTQDMLMLIMAREILKAKSQNKDFTKDFCLKYVMTDSLLDKPIDFDWSVNIEKKKK |
| | NEEGKIEKEIIRKTIRQEGMKMKNYGQFYKFASDHQRLESLLSRLPDELFLRAEIENELSYYDTNRSEVFRLV |
| | YIIESEAYKLKPELANDANTDKEWFYYADKKGKKHPKRNNFLSLLEILAAGKDGILNEDEKRSLQSTRNAF |
| | GHNTYDVDLPTVFEGKKEKMKIPEVANGIKDKIENQTEELKKSLQK |
| IMG_ | VEEVFNLLRRKSNPPQSKSQLDCDIHEYVEKHRKDKSKEWAEDPTALMRKQAKQVEQTEHAIRRCQIEDIV |
| 3300032030_2 | MLYAARDMLYAARDILSAKNNRTPNGTDTPQPQKFKLKHVQKDDGLLERTIDFDWVVDIDGQQKTIRQQ |
| SEQ ID NO: | NMKMKDYGKFYKFASDGERLKSLLAHLGGNEFQRADIEAEYANYDVSRSQVFRYVYMLESKAYQLLAK |
| 4586 | RKDNPIDLLNDQKPIPDAFWFVSKEGIRNEFRVFKENLFNEYKADVHDYKANDPDAEALVNAICNFTGTSIE |
| | EWDANQDTLMKQVKSLLTDENPNEKDKALILQVKDYCMKKDIARKAIRNNFGELIEILLRGDEPIFTDDDK |
| | YIIQHIRNAFGHNHYLKKEDEYNTVFRGKEAKLKLPEVAKTIKDWMGEKTTKALSLTPDTQRALPPKSQAA |
| | E |
| GCA_ | LIYLQEKVNKTIDVRNYKTGKTSTIDKSWMMTTFYKREWNQEVGKQLTEVKLPDNLSGIPFTLRQLKEKAS |
| 002400765.1_ | YSLDQWLNNVTKGKVAGDGKRPINLPTNLFDETLINLLQNDLEAQQVEYPTDAKYNELFKIWWRKRGDST |
| ASM240076v1_ | QSFYNAEREYVIFDEKVNFKLQENAMFTDFYSDSLKKAFRAKQNTRRIEQRSNRRLPDIQFSQVEKVFKRSI |
| genomic | SNTEKQIRLLKEEDQIMLLMLEELMSSDLDLKLNQIDTLLNKTITVKKPVTGNLSFGDKSEITRTIIDQRKRK |
| SEQ ID NO: | DHSMLHKYVYDRRLPELFEYFEENEIPLQDLKNELEAYNTAKQMVLDAVFKFEEDIVTNNQVHDLIGSAC |
| 4587 | DTGHIQHKVYLQWLKKEGMINENEYLFLNRVRNCFSHNLFPQKRTMSLFVNQWADSNFALQIAEHYNEKI |
| | NAILAI |
| GCA_ | MLTPLNFEPLRKKYIKKKEENMSHPPGYPYSKEEFKDLKERHEKLQKFLDTDYKGLKVWHLPDDIKEYLIQ |
| 900113045.1_ | VSTPSYRQLALIKIDEIKAQTKQLIKDCKALEKKKPEERTLRAGHIAQILARDMVYLRKHHQHIKDGKVHHS |
| IMGtaxon_ | KLNDEEYNRLQNLLAYFQKDDIQAHLDQYRLLELHPFLKDVKLENSKNLYEYYKKYLEKRKEYFIGVYDS |
| 2636415974_ | IDLFAPIFKKAEQYLKKFHGVNKDEKDKKVKKSLLELKKALFNFDRQVIKIFLKNYITDWKGINLGNCKNA |
| annotated_ | EDFCFHVFSIEKEFKDKTFFNIDELQNKLGYLFDFKYQENQPEKLDKNYAQIPIALPSSLFKDAIIEGIEKAMQ |
| assembly_ | KEGKKLTFGKDEEGNEKRTVVYALREWLENDTQVFYTHNRHYKMPEKSKEIFSKEQIKRIECQVISLENRK |
| genomic | ELYHYKELSLYEWIDKPEKYDKLKEIAQKDALKKFRSYLLSTEKVLRLEQHKDRVLWLLAKDLSENRTQG |
| SEQ ID NO: | TNLDFKNFKLKDLEKFLDTPIKMEVKVNYIVDKEDIAVNNAISGKPCTSEKQLIEVGAVSETLKIKDYGDLR |
| 4588 | RFAKDRRLPNLFRYFYDVKSKGEALSKTELEKAITLLEVRNLIQRNEDGSKIFKDKITVLEKAMELEQLLHE |
| | KYGQPDTDFENTRLGKYWKQEQPKDSHISHNTYIKFLQDKTLGIDLNINPIVGMDGAQHLNNDKELAKSK |
| | RTHPEQTLEACLIILLRNKLIHNEVPYTTFLQSKMQADFTPEQVVRQIIDESMRIYDQLIAEVKKQTA |
| IMG_ | LPKLLALQLLTRTAEQPIINFIQTTNAKIFNFGELEKIKDRTNYIPEVFTKRVVRERELINNKGGITYLSNKLE |
| 3300028769_2 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4589 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300028862_4 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4590 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300028767_4 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4591 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300028774 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4592 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300028738_2 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4593 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300028739_2 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4594 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300029998_4 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4595 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300030055 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4596 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300028864_2 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4597 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300030047 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4598 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300030491 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4599 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300030048 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4600 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300030001_3 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4601 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300031918 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4602 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300030000_2 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4603 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300030002_2 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4604 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300030673 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4605 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300029981 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4606 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300030943 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4607 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300030685 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4608 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300028853_2 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4609 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300029995 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4610 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300028650_2 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4611 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300030294_2 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4612 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300029923_2 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4613 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300029989_2 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4614 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300030339 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4615 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300028676 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4616 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300029983_2 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4617 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300030019 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4618 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300029990 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4619 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300028772_2 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4620 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300030838 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4621 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300031521_3 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4622 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300031722_2 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4623 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300028763_2 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4624 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300030230_2 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4625 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | LPKLLALQLLTRTAEQPIINFIQTTNAKIFNFGELEKIKDRTNYIPEVFTKRVVRERELINNKGGITYLSNKLE |
| 3300028734 | MNILNKYHLSIVQLAAMDKDSFKKLTGKNKKDIELLSQIKYQYLLKQRREELKKHLPTGLNLDMLPKRVL |
| SEQ ID NO: | DYLMQSNERNEKKKIHQKIKDIKDEAKLLNKSIKKEIEKTQQEQRIKLGEIATFIASDMLNMIIDKELKSRITN |
| 4626 | PYYNKLQNKIAYFSLNKGEIISICDELSLFDTKKGHVFLTRNLIFKSTGIIDFYQNYLEAKIAWIDNKLFVKGK |
| | MGGYTIPNNNVPYSILKIKNEIRTYNFNTWLKNKIISPVELPNSLMDDQLAKVLKSALKKESKTYNDGDKFS |
| | ILLAKYLSNDSQPFYEYTRNYTINTEPVCFDVQGLSSKDIQDKYGNRVAENEKLIRFIQTKDRILKLVCDELL |
| | LKDPSIGSKDRFKLSEIHPTSLHNPLERQAEFKHKIIRKGTEVFFTVVAKDTKEQIQEVIEYEKITSSDEKEKY |
| | PGQKWYQWTIKDFGRFKRFLKDRRLPNLSEYFEDKDITFDFLEYQIKEYDKYRNNVFDLTFELEKSIATSDL |
| | EGIRQIEQRLHIRPKGFFEIEFDVYVEWLDRKGIQYNKDLINECRNRFSHSEFPKYEHLNQIPKITRQQIIDFEY |
| | NKRTREYKNNADISISEKITNQYKTEIEQIIHQIRLQ |
| IMG_ | MAFWGREQDKLPNIFGQLNLLQQHPFLNGVELKDVGIYTFFRNYFEQKVSHLNSVLRKIETKTATIEDYYFI |
| 3300000983 | NLKERTDIEERVNNLLYTQEEDVEPMIQLPGDFFYQQLLKHIEQHLPEFYTILTANEGNPARLNYVIEQYFK |
| SEQ ID NO: | HHHKDEPQTLYNKKRSYHTYNQWLDKRTKKTMRNALEKEFLSVDDRTKDYQKIKEALKDKAAEATVKR |
| 4627 | GVVEKTKNIWWKGLKNINQNESRLRNIKAEDVLLYLAAMKIFKAELPELKLATEVKLKNLSAADESSLLEK |
| | QIPFELPYAFIDENETAQTIWITDTMKMKDYGKFRRFLKDRRLPNLMDMMNQFRNKFSHNQYPPKSVCGF |
| | AVDKHKDDKIAAQLAKAAIEIYENTVKKMNTST |
| IMG_ | MNAIELKKEEAAFYFNQARLNISGLDEIIEKQLPHIGSNRENAKKTVDMILDNPEVLKKMENYVFNSRDIAK |
| 3300031651 | NARGELEALLLKLVELRNFYSHYVHKDDVKTLSYGEKPLLDKYYEIAIEATGSKDVRLEIIDDKNKLTDAG |
| SEQ ID NO: | VLFLLCMFLKKSEANKLISSIRGFKRNDKEGQPRRNLFTYYSVREGYKVVPDMQKHFLLFTLVNHLSNQDE |
| 4628 | YISNLRPNQEIGQGGFFHRIASKFLSDSGILHSMKFYTYRSKRLTEQRGELKPKKDHFTWIEPFQGNSYFSVQ |
| | GQKGVIGEEQLKELCYVLLVAREDFRAVEGKVTQFLKKFQNANNVQQVEKDEVLEKEYFPANYFENRDV |
| | GRVKDKILNRLKKITESYKAKGREVKAYDKMKEVMEFINNCLPTDENLKLKDYRRYLKMVRFWGREKEN |
| | IKREFDSKKWERFLPRELWQKRNLEDAYQLAKEKNTELFNKLKTTVERMNELEFEKYQQINDAKDLANLR |
| | QLARDFGVKWEEKDWQEYSGQIKKQITDRQKLTIMKQRITAALKKKQGIENLNLRITTDTNKSRKVVLNRI |
| | ALPKGFVRKHILKTDIKISKQIRQSQCPIILSNNYMKLAKEFFEERNFDKMTQINGLFEKNVLIAFMIVYLME |
| | QLNLRLGKNTELSNLKKTEVNFTITDKVTEKVQISQYPSLVFAINREYVDGISGYKLPPKKPKEPPYTFFEKI |
| | DAIEKERMEFIKQVLGFEEHLFEKNVIDKTRFTDTATHISFNEICDELIKKGWDENKIIKLKDARNAALHGKI |
| | PEDTSFDEAKVLINELKK |
| IMG_ | MNAIELKKEEAAFYFNQARLNISGLDEIIEKQLPHIGSNRENAKKTVDMILDNPEVLKKMENYVFNSRDIAK |
| 3300031365 | NARGELEALLLKLVELRNFYSHYVHKDDVKTLSYGEKPLLDKYYEIAIEATGSKDVRLEIIDDKNKLTDAG |
| SEQ ID NO: | VLFLLCMFLKKSEANKLISSIRGFKRNDKEGQPRRNLFTYYSVREGYKVVPDMQKHFLLFTLVNHLSNQDE |
| 4629 | YISNLRPNQEIGQGGFFHRIASKFLSDSGILHSMKFYTYRSKRLTEQRGELKPKKDHFTWIEPFQGNSYFSVQ |
| | GQKGVIGEEQLKELCYVLLVAREDFRAVEGKVTQFLKKFQNANNVQQVEKDEVLEKEYFPANYFENRDV |
| | GRVKDKILNRLKKITESYKAKGREVKAYDKMKEVMEFINNCLPTDENLKLKDYRRYLKMVRFWGREKEN |
| | IKREFDSKKWERFLPRELWQKRNLEDAYQLAKEKNTELFNKLKTTVERMNELEFEKYQQINDAKDLANLR |
| | QLARDFGVKWEEKDWQEYSGQIKKQITDRQKLTIMKQRITAALKKKQGIENLNLRITTDTNKSRKVVLNRI |
| | ALPKGFVRKHILKTDIKISKQIRQSQCPIILSNNYMKLAKEFFEERNFDKMTQINGLFEKNVLIAFMIVYLME |
| | QLNLRLGKNTELSNLKKTEVNFTITDKVTEKVQISQYPSLVFAINREYVDGISGYKLPPKKPKEPPYTFFEKI |
| | DAIEKERMEFIKQVLGFEEHLFEKNVIDKTRFTDTATHISFNEICDELIKKGWDENKIIKLKDARNAALHGKI |
| | PEDTSFDEAKVLINELKK |
| IMG_ | MNNIELKKEEAAFYFNQAELNLKAIEDNIFDRGRRKTLLDNPRILAKVENFIFNFKDVTKNARGEIDCLLSK |
| 3300032053 | LTELRNFYSHYVHNDNVKILSKGEKPILEKYYQIAIDATASANVRLEIVDNGNKLTDAGVLFLLCMFLKKS |
| SEQ ID NO: | QANKLISGISGFKRNDPIGQPRRNLFTYFSVREGYKVVPDMYKHFLLFALVNHLYNQDDYIENIEKAQQPSD |
| 4630 | IGKGLFFHRMASAFLNISGILRNMKFYTYQSKRLKEQRGELKHEKDIFTWTETFQGNSYFSVNGQKGVIGE |
| | DELKELCYALLIGKQDVNEVEGRITQFLKKFKNADNVQKVEDDEMLDSENFPANYFAEPAADNIKDKILNR |
| | LKKAIESYKDAGADVKAYDKMKEVMTFINNSLPADEKLKRKDYRRYLKMVRFWGEEKGNIEREFETKEW |
| | SKYFSSNFWVAKNLERLYGLAKEKNAELFNKLKATVEKMDEWEFGKYQQINKAEDLAGLRRLAKDFGLK |
| | WEEKDWEEYSRQIKKQITDSQKLTVMKQRITAGLKKKHGIENLNLRITIDSNKSRKAVLNRIAIPRGFV |
| IMG_ | MDRIKNRTQHLRAEDRATFLSKDLVYFQPHTTAKDGPNGDEFRKLQAATAFFERDKEYFIRMFHQCKLNDI |
| 3300020661 | GTCHPFFTEDDIRKCKTNNDLYERYLKKRHDHLVYCKKLNKKATDIPIELKFLKIKNTIADDKALQDLAKE |
| SEQ ID NO: | VKKNAINLPRGLFKDATVKMIELYGNDAMKNAVATSKKNPKSETPDHNTANTVYLVQKYFEQMNDSYQP |
| 4631 | FYDYDRNYRTVDEWYDNRNDGAMTQIAHVPQTKKELTTLAGTIKDSERKFNDKATYNQYLRKKAEENKP |
| | TRHVSNHNNRNSSIKIIEVTPAQKLESIIDDRDFYKVYKDKVIENEKLIRHYRTGDQMLFLMCIEFIDDPTFD |
| | KHIKEILRKERDAGHFLLKEIKPKNKKSILNTPIPYELPLTVKSKEDGKEEEKRIKSVIKIKNYGGFRRFLKDR |
| | RLDSLLTYLPGNVTERDHLEWQLSNYENKRIDIFKTIYEFEKACGENVHYKKKIAELLIKPGRAAHKHLMTK |
| | TIEIIPTISSTLQERLNKIRNAFSHNQFPPFLFVKPILKGDDSFIEQIVKYTTAEYDSYIAIIKK |
| GCA_ | MTYFNIHIRPNHFSAEESSQKLKKRHNALDTLLKIERVFKESLTCDLTTSPLISQQDLLDRAKQICDHYVGKQ |
| 000829755.1_ | NKLPWILRMIFSKKKMVEATYERIVRLSTPPLPVSEEVASHALSFLDGSSLGEYSQVSHWANRQALEAQFSL |
| ASM82975v1_ | VRRYGYEGNDINEAKKYIHNLRQEMHFLSKNEWKLAPDSIFSLKNLIIYEKRQLNFKQSWQNLMQISVNDV |
| genomic | AKLYSKESCPELCRELLRINLSTPDQFTDQTSADFALMIAVRRQDAPAVQFFLSHGANPDFTFNKISMISLSI |
| SEQ ID NO: | NRKAPAITKLLLEAGADIRQMSVSRHSLFEQILRTPDNVELLKCLIDCGFDVNQPIRDGLTALHVAAELGNL |
| 4632 | QMVELLLEHGAILDATNSEGDTPLLLSIASPKIVELLLQSGANANHANAKGRTALHYAMIVTHPILADKLSQ |
| | SAEILKKYGADPDLKDNDGLTPSEYSARALG |
| IMG_ | MFFNMAVNNVHTVVQFLERKYALSLDDAPPPNDEAEDRHREEREEKILGSEKKKIKIPASPIIEVLRKGTDIS |
| 3300027984 | LQANIIRDLIHYFPFLREVKNHREKPYAYEKNKETKKKPAKQLSPGEIAELFLFYAKLLYDQRNYYTHACSK |
| SEQ ID NO: | PTPLDFTGKKLYDLERIIEYNRRTVNERFFKGKVPNSQAEIELLPLTPKINIQQIISQTKNQLNLVQQEICETRD |
| 4633 | QRNLREKCGQRLSTLLSLLDAQRNQNEQNELSALDDQCEQNKLSDNPVYLLGQKFFKDDNTDLSAMGRTF |
| | VIAQFLEAKYISQMIQQLVDAGELSIPECVNSDADNKLLITRAFSITHIVLPRTRLQTDNVLNPMSIGMDSLC |
| | ELHKCPEALFDMISTENQGKFRFFDDKEGIENLFKRFGKDRFAYLALNYLDMSNRTREDASPDEKTTGKFE |
| | RIRFHIDMGNFFFTKYEKENMIDGTKLKHRRLSKRIYCFK |
| GCA_ | MKKELNREDVFTGGKTPELTIYYNIAYFRMAGLINALCGNKLERDKDALEQFMKAFINGKQKLTDMQFVK |
| 003513165.1_ | LCDYLWKGYKYDKNRSEYSLTENDKTMVLKMVAKLQDIRNFQSHIWHDNKVLVFDADLVQFIKNKHLE |
| ASM351316v1_ | AIDAQRQRFSKETEVYEKENRELKKKKNKELLFDIHDGLGYITGEGRNFFLSFFLTRGEMTRFLKQRKGCK |
| genomic | RDDTPEYKIKHLVYRHFTHRDGATRTHYGYEDNMLDQLPDKQEFLTTRHTYRLINYLNDIPEEVTNPELFP |
| SEQ ID NO: | LFNTISNGILIKESVGTYIAFINKMPLLSDFEFSEVWGKDGKPYTNLVEFYHKTQPGFKFRTDLNSFHKIILNL |
| 4634 | IRDENYTGFFTTQLNLFMAHREELILVISKPLITPNDLLLIEEHYRYKLKANDFVREKLGEWKEKIEKGKWE |
| | KANEQKDKLINLLGGVIEITFYDFYWQKDEKPRNENRFMEFAIRFMADFNLLPDCEWEVEPLQIENDLIANR |
| | TATPDLKKSGSQFMNQLTDNYRLKFNDGQIAFRYQNQVFIMGHKAVKNLLIACFDGKEKMLNGFMPALL |
| | ADLKKITNEHILKNHQALNTLSLLNNDSIPSYISRQWGEAEPITDMKKKAIARMDYITQQFEALIENHHYLN |
| | RADKNRQIMRCYKFFEWQYPQNSQFKFLRRNEYHRMSIYHYCLDKEQHKYDKKGHNYLYNRLIKESHNE |
| | SGNIEQHLPYQIRTMLNDAKDFNDYFLRILNATHKILTDWKNQLKQGREPNNYYLSRLGFTGGLTQKVVH |
| | TRLLPFSIHPGIPVSFFYRTEMNQNPSFNLSAKVWNSESPFRVGLKESNYQYAKYLGLFNEIKTQRKIIGKMN |
| | QLIAEDALLWQIAKKYXX |
| IMG_ | MKKELNREDVFTGGKTPELTIYYNIAYFRMAGLINALCGNKLERDKDALEQFMKAFINGKQKLTDMQFVK |
| 3300025154 | LCDYLWKGYKYDKNRSEYSLTENDKTMVLKMVAKLQDIRNFQSHIWHDNKVLVFDADLVDFIKNKHLE |
| SEQ ID NO: | AIDAQRQRFSKETEVYEKENRELKKKKNKELLFDIHDGLGYITGEGRNFFLSFFLTRGEMTRFLKQRKGCK |
| 4635 | RDDTPEYKIKHLVYRHFTHRDGATRTHYGYEDNMLDQLPDKQEFLTTRHTYRLINYLNDIPEEVTNPELFP |
| | LFNTISNGILIKESVGTYIAFINKMPLLSDFEFSEVWGKDGKPYTNLVEFYHKTQPGFKFRTDLNSFHKIILNL |
| | IRDENYTGFFTTQLNLFMAHREELILVISKPLITPNDLLLIEEHYRYKLKANDFVREKLGEWKEKIEKGKWE |
| | KANEQKDKLINLLGGVIEITFYDFYWQKDEKPRNENRFMEFAIRFMADFNLLPDCEWEVEPLQIENDLIANR |
| | TATPDLKKSGSQFMNQLTDNYRLKFNDGQIAFRYQNQVFIMGHKAVKNLLIACFDGKEKMLNGFMPALL |
| | ADLKKITNEHILKNHQALNTLSLLNNDSIPSYISRQWGEAEPITDMKKKAIARMDYITQQFEALIENHHYLN |
| | RADKNRQIMRCYKFFEWQYPQNSQFKFLRRNEYHRMSIYHYCLDKEQHKYDKKGHNYLYNRLIKESHNE |
| | SGNIEQHLPYQIRTMLNDAKDFNDYFLRILNATHKILTDWKNQLKQGREPNNYYLSRLGFTGGLTQKVVH |
| | TRLLPFSIHPGIPVSFFYRTEMNQNPSFNLSAKVWNSESPFRVGLKESNYQYAKYLGLFNEIKTQRKIIGKMN |
| | QLIAEDALLWQIAKKY |
| GCA_ | MKKELNREDVFTGGKTPELTIYYNIAYFRMAGLINALCGNKLERDKDALEQFMKAFINGKQKLTDMQFVK |
| 003518305.1_ | LCDYLWKGYKYDKNRSEYSLTENDKTMVLKMVAKLQDIRNFQSHIWHDNKVLVFDADLVQFIKNKHLE |
| ASM351830v1_ | AIDAQRQRFSKETEVYEKENRELKKKKNKELLFDIHDGLGYITGEGRNFFLSFFLTRGEMTRFLKQRKGCK |
| genomic | RDDTPEYKIKHLVYRHFTHRDGATRTHYGYEDNMLDQLPDKQEFLTTRHTYRLINYLNDIPEEVTNPELFP |
| SEQ ID NO: | LFNTISNGILIKESVGTYIAFINKMPLLSDFEFSEVWGKDGKPYTNLVEFYHKTQPGFKFRTDLNSFHKIILNL |
| 4636 | IRDENYTGFFTTQLNLFMAHREELILVISKPLITPNDLLLIEEHYRYKLKANDFVREKLGEWKEKIEKGKWE |
| | KANEQKDKLINLLGGVIEITFYDFYWQKDEKPRNENRFMEFAIRFMADFNLLPDCEWEVEPLQIENDLIANR |
| | TATPDLKKSGSQFMNQLTDNYRLKFNDGQIAFRYQNQVFIMGHKAVKNLLIACFDG |
| IMG_ | MKKELNREDVFTGGKTPELTIYYNIAYFRMAGLINALCGNKLERDKDALEQFMKAFINGKQKLTDMQFVK |
| 3300009296 | LCDYLWKGYKYDKNRSEYSLTENDKTMVLKMVAKLQDIRNFQSHIWHDNKVLVFDADLVQFIKNKHLE |
| SEQ ID NO: | AIDAQRQRFSKETEVYEKENRELKKKKNKELLFDIHDGLGYITGEGRNFFLSFFLTRGEMTRFLKQRKGCK |
| 4637 | RDDTPEYKIKHLVYRHFTHRDGATRTHYGYEDNMLDQLPDKQEFLTTRHTYRLINYLNDIPEEVTNPELFP |
| | LFNTISNGILIKESVGTYIAFINKMPLLSDFEFSEVWGKDGKPYTNLVEFYHKTQPGFKFRTDLNSFHKIILNL |
| | IRDENYTGFFTTQLNLFMAHREELILVISKPLITPNDLLLIEEHYRYKLKANDFVREKLGEWKEKIEKGKWE |
| | KANEQKDKLINLLGGVIEITFYDFYWQKDEKPRNENRFMEFAIRFMADFNLLPDCEWEVEPLQIENDLIANR |
| | TATPDLKKSGSQFMNQLTDNYRLKFNDGQIAFRYQNQVFIMGHKAVKNLLIACFDG |
| GCA_ | MYTKEQKERVPPTRKELYMGGKTKTADYAVFYNIAFNGILSKIHFKETGQTEFDEAKLSVMYGQKYLDVF |
| 003165435.1_ | ETARPSNIKHDFKDETYLTLRDFLWKGYETDKNSSGHALTDEDKSITRALLVKLRVIRNYHSHIWHNNDGL |
| 20120700_ | KFDNSLQIFIKKKHDDAINSLYATHPAEVDAYKEESKQAHLFKDNYITTEGRVFFLSFFLTSGEMSSFLQQH |
| S1D_genomic | RGSKRTDELKFRIKHIVYRYYTHRDGSTRQKFSQEDDVLSSFSPNDQQDVLMARQSFKLITYLNDVPDSAN |
| SEQ ID NO: | NIDLYPLFTDSGERKPAETAQELKAFCDQRELFTTIQITDVANKKGEIQTGVLNFSIPALGDTAVRLGRGTFH |
| 4638 | KLIIDTLRRHDNGKFVYDGLTWLITERLKLIEELQKLDTLQHLDETSGMAQRKFFEEYMLHKLNGNLYLQQ |
| | LMRNWFYAFEKDLKKEGKLRDKLVQGCIQDPVAPGYYDFYFEEGEKPRNTDRFSEFAVKYLIDFNLAPDW |
| | EWMMESFGVADKHGKAISGKNKAFFSHFKSGTAWRLSVTDGCVIVRLKRLPDFRFQLGHRALKNLLIAHF |
| | YQKANIGTLLNKLTKDTQKIRNVLYNKTSHTLTDLALLERKNLPRFVLLTLGDAQTAAGETVENATVATL |
| | KRISNLIAKLRDWRTNHKTISRNEKNRIIMDCYQLFDWKYPDTGTYKFLRRDEYQNMSIYHYMLERVLNLR |
| | EDITYFQQKIQDAEDYGKKKKYQMAIFDNNKQIERTETILSKLLKDVKNRIPEEVNQILENSESLDDLLKNA |
| IMG_ | MDYPQRKAKPQQHFKGKSSKPNRFSPGTRTGRRPSSEYDFFTGGGTKITNTDKESTKTADFTIFYNIAFENV |
| 3300020782 | EKVKTQLQNKTQSQQNAFWAEYFWKAHFLSKNTNGYELTQQDDRIITQLIKKVEEIRNYHSHIWHDNSVL |
| SEQ ID NO: | VFSDELKRFVEQKYNEALVQLSVDFPGAVSDYQFLKQKNYEKESKLFNPIGFGGDAKNFITIEGRIFFLSFFL |
| 4639 | TTGQMNQFLQQRTGYKRADMPQFKIKRLLYTFYCNRDGAAITDFNHEDRFIDTLAPEVRQNVFKARTAFK |
| | LISYLMDYPDYWGSNDAMPLFDNNNELIKNVEQLKDYIESKNILPELKFTLIDRKIKASLELEEDAETLKKE |
| | QEDKHSTGTIAFTYDELSGFSFHINFEALHRLVLLQTLHHNMVDLPAPLAILAIELKKQANNRTTLYDILIKPI |
| | AERTDDEQVYLLTKENQYLRGGRKVTELGIIFF |
| GCA_ | MHPKKSGSAPTAYEAFTDFGDKTADLAIYYNIAVANLNEIRKAINASTANPDTQLARLAEYLWSAHKDAK |
| 001870995.1_ | NSRGYELTPDDKVIINELCKKVEDIRNFYSHQWHDPCVLECSGQLVSFINDRYKLAAAMVAKDDPAAVAD |
| ASM187099v1_ | YEALLGKREYKPYKLFNGSVLTVEGRIFFLSFFLSSGQLSQLMQQRRGFKRTDMPLFRAKRKIYLYYTLRD |
| genomic | GATMAHFHQEQSVLSTLSAEDQKQVLKARTAYKLISYLNDYPDFWGNTEKMPLYLSKGKKIENIDNLYEY |
| SEQ ID NO: | LQQHPELLPDFEFAPPDEEETGIRKHILFTHEGLPGYEFKMDFPMLYRLVVLMQLFNATVLQESPVDTLLQN |
| 4640 | LRTVIEAGRSYSTF |
| IMG_ | LRPVPHRRPREIPQGRARKRGRLPEDRGGDVRRGRRPHDTYQGVRILAYAASRHTAHRNRPEPRFGDPGAT |
| 2049941000 | GVAACRYRRGPARRHHRDRRRRRGSHRGRCAAQDRERLYAGAGNHPVVRDQAGCAADAHHELQHVRG |
| SEQ ID NO: | LPHPSSLQHLHXXXXVLPEAKKRELEAVNIDEETVIKKRHSDRFVPLLLRFIDANELFPSIRFQVNSGYLRFL |
| 4641 | HHEKAAYMDGVERPRILQDAVNGFGRIQEVERKRSASDTYLGFPLYAPSPDDDVTMPLPCITESASXXXXX |
| | XXXXXXXXXXXXXXXXXXXXXXXXXXXXXXXXXXXXXXXXXXXXXXXXXXXXXXXXXXXXXXXX |
| | XXXXXXXXXXXXXXXXXXXXXXXKHDRPRVRQHRRLLCAIEGLRQAGDRPELGVRTGAGRRGIGRAGT |
| | CGRFRRTIGGRESRPVAPETEGQVPEHVEEGHRRRARTGRRGEEALGREHRPGDXXXXRPSTTSRASTSTCS |
| | TSPLPARLSSGAWNRRSSWPTVAPTPTSTTCRSRGSIPGWSRRTSPSRSSSIPXXXXKVDMGTYPYPETTYA |
| | KVYVVRESNISAYDFVSTGRLVDVGRSKSNPHGFMIERFQVVKNERTETRRRN |
| IMG_ | MAVYLAKSIVNLIKNNELKPTGKNYNVMQANLAVYESFDKVKQMFIRSKMVDSHPFLKNVLQRSPQRAID |
| 3300028886_2 | FYKFYLSEEKAFFSNINNCTYLKFYKNRISKYDDKGAAYYKNLAEHYLKNGFVELTNHIFADEIRAKLSGK |
| SEQ ID NO: | MADIDANDQKYNISFLISRYYKESQPFYFFNCVFNNEIDDPKTKKHIEKAITRYRIQDKILFAMAKTLLKTQD |
| 4642 | DVSKAQISDIAEMTLSNIMPGDNIFDKPMQNFSVKVTLDGKQLTINADNIKVKNYSTIFKTIFDRRLDTLAKL |
| | ITTPQVSLTDITAELELYDREALKINKLVYDTDDAHRAIPLVKVIRNSFAHWSYPHKDSLRNKHGVISDAYV |
| | TNLHNKLVNKNLGEKVGLIKPELEMALM |
| UYCW01.1_2 | LDKVLPEAYSVCCPRNTIEFYERYLEEYQRYLKPLVIKLEKGKVPSLSFVNEGQRRWAKRDDAYYHELGNL |
| SEQ ID NO: | YLSQAIELPRQMFDDEIKDKLREMPEMRDVDFDHANVTFLIGEYLKRVRHDESQEFYSWPRHYKYVDMLK |
| 4643 | CILNSKNGSLQAVYTQMGEREGLWQERSELEEKYAKIRLRDLGRKGLDKDEANERIKTGLGNRKKEYQKA |
| | EKVIRRYKVQDALLFMLAKNTLFNSVEVDDERFKLKDIMPDGEKDILSEVVPMDFCFRSGNSATRKLMGTI |
| | HSDNTKIKNYGDFFALANDKRMVTLLPLVGEQCLVKEEVEEEFDKYDDCRPEMISMVFDFEQWAYSAYPE |
| | LKELVSNEAIKGRLFSNLLQELLGRGELTYEEKYALVGIRNAFLHNSYPKDGGVVKVRTLPDIAKSLKDVF |
| | KEYIRLE |
| IMG_3300014786 | MKKRXXXXXXSRMISGEASYPVRFSLFAPRYAIYDNKIGYCHTSDPVYPKSKTGEKRALSNPQSMGFISVH |
| SEQ ID NO: | DLRKLLLMELLCEGSFSRMQSDFLRKANRILDETAEGKLQFSALFPEMRHRFIPPQNPKSKDRREKAETTLE |
| 4644 | KYKQEIKGRKDKLNSQLLSAFDMDQRQLPSRLLDEWMNIRPASHSVKLRTYVKQLNEDCRLRLRKFRKDG |
| | DGKARAIPLVGEMATFLSQDIVRMIISEETKKLITSAYYNEMQRSLAQYAGEENRRQFRAIVAELRLLDPSS |
| | GHPFLSATMETAHRYTEGFYKCYLEKKREWLAKIFYRPEQDENTKRRISVFFVPDGEARKLLPLLIRRRMK |
| | EQNDLQDWIRNKQAHPIDLPSHLFDSKVMELLKVKDGKKKWNEAFKDWWSTKYPDGMQPFYGLRRELNI |
| | HGKSVSYIPSDGKKFADCYTHLMEKTVRDKKRELRTAGKXXXXXXXXLSLLTWLPTSSGAFTELSMNVNL |
| | CSASCKKTIGLC |
| mgm4547164.3 | LPRLLSSXXXXXXXXXXXXXXXXXXXXXXXXXXXXXXXXXXXXXXXXXXXXXXXXXXXXXXXXXXX |
| SEQ ID NO: | XXXXXXXXXXXXXXXXXXXXXXXXXXXLFYQHLRKEDNLSGGDFPSAEDIIIKQYDNLVRFFKEVKDIQP |
| 4645 | NNDEPELAKILSTFGLSKQSVPKKIYDYLSGKATAISKDFNKSAGIELNRRLNKAKGKLREFLADKDKIESA |
| | DNKYGKDSFASIRYAQLADYLAESIMDWMTLKLTGLNYRVLASSLAAFGTRQTDDDIMQLLADAHIYKD |
| | AVTDHPFIAWTLGDDDNPVTDIETFYESYLKREIKQIEKYISVTTDPKTNKDVITLKKNPEDIPFLHRQRRRW |
| | QENTIAEQAERYLFIAEEGKEKPSRATLLLPDGLFTPYIIKVFELKHPELIMQLESLTDKQXXXXXXXXXXX |
| | XXXXXXXXXXXXXXXXXXXXXXXXXLIMQLESLTDKQKIGITNNAAYLINLYFESKGEMSQPFYDSTEPS |
| | YYNDSIRKVAPYKFSRSYDFFNTLKGWQIHLPVDKIKKQLLQKDTLINNQVNQLSVKGNFDSLEDAKDSLK |
| | RRLLRDLRDMQDNERAIRRHKTQDRILFLMAKDIMGDTVSQNGDLFKLENVCKEEFLNQKVPVKHTVRSG |
| | DKQMVIQKEEMPIKDYGKIYRLLSDNRMVALLSYTLFANGDTINYDHLSEELKQYDLHRSSALKSAQVLE |
| | NKRFEQSREVLTDPERDEFYQGNRRYKNRARTKENEAKRNNFSTLLKDLQKLTPEQMEMFSKEDRQLIIAV |
| | RNAFCHNSYPSWDVVNKLLIQSQSERPDLELELTQIANFLITKLSGYVKQAENN |
| mgm4547164.3_ | VILFTYTKRKLXXXXXXXXXXXXXXXXXXXXXXXXXXXXXXXXXXXXXXXXXXXXXXXXXXXXXXX |
| 2 | XXXXXXXXXXXXXXXFGSCKVLLPDPEVDSNKSKLGLSDFGMLYFCALFLSKEQLVQFCTEAKVFVNSPF |
| SEQ ID NO: | NNDNNKKNNIILNMMYVYQTHIPRGKRLDSERDSQALAMDMLNELRRCPIELYNVLPAIGKREFEDNVIHQ |
| 4646 | NSRTPELSKRIRTKDRFPHLALRYIDSQHLFEKIRFQVRLGSYRFCFYDKVCIDGKTHPRQLHKEINGFGRW |
| | QDMEKERKEQYGPLFQKTREESIWQKDENAYVNLRQLEPIKAGDPPHITDTITQYNIHKNRIGLYWNTSGE |
| | TYLKDKASEQGETICQGYYLXXXXXXXXXXXXXXXXXXXXXXXXXXXXXXXXXXXXXXXXXXXXXX |
| | XXXXXXXXXXXXXXXXXXXXXXXXXXXXXXXXXXXXXXXXCCSTSTFVRKTILVEVISLLLKTLLSNN |
| | MITLSDSLRRLRTSSPTTMSRSWQRYSALLVSANKAYPRKFTIICQGRRQPFQRISISLQE |
| mgm4547164.3_ | VIAPDLTPTEETCDGLSAFGMLYFCSLFLSKEQTAQLCTESRVFVTSPYQPAGNLKNNIILNMMFVYAIHIPR |
| 6 | GKRLDCENDTQALAMDMLNELRRCPRELYNVLPAIGKRDFEDNVIHENNRTPELSKRIRSKDRFPYLALRY |
| SEQ ID NO: | IDQKGLFEKIRFQVRLGSFRFCFYDKICIDGKSHPRQLHKEINGFGRLQDIEKERKEQYGPLFQLSREQSVWQ |
| 4647 | KDENAYVNLKQLEPIKAGDQPHITDTFAQYNIHQNRIGLFWNTDEESKLVNKTNSQGEIICDGYYLPPLNSV |
| | DSPTEHKRKALVEMPAPLCSLSVYELPAMLFYNYLRSIDGLKGEVFPTVEEIIIKQYDNLRNFFKEVTNIQPT |
| | DNIENLTAILNAYGLSKHSVPKKIFDYLSNKNTSINKDIWKSAEIEVKDRLRRAIIRKQCFEKDQERIENTKD |
| | NKFGKDSFACIRYARIAEELTKSMMEWQSENSKMTGLNFRVLTASLAKFGDGVTKKDTIISMLRNAKIMGG |
| | DHPHCFIEQAVELEQDDIEDFYLDYVSAEIQYLTRFLTIDDQAIELEEKQLLDALRKDKEARDDARIHLKND |
| | VDFDELPFIHKSRLRWQQSKIAELANRYLYVKEEGKETPGRATLLLPDGMFYPYIMKVFEQCHSELMNNIN |
| | ALSDEQKKGISNNAAYLINLYFESKGEKSQPFYDSTEPSYYNDNIRQLAPFKYARSYEFFKIIKGWQIHLSCA |
| | EIRNKLTGYKTLINNKVNGLTEKGNYISLEEAKNALRRRLHNTFYDMQDNXXXXXXXXXXXXXXXXXXX |
| | XXXXXXXXXXXXXXXXXXXXXXXXXXXXVPFVVIRRKIALFSSWRKI |
| mgm4547164.3_ | MHSVXIRNNENSEEAISLLLDIVKRCYDERWNVEKDALTGLNDHQKKNKEDEFYAQCSDEGATGEKVHLL |
| 3 | TAFTLRSEQEERLRKLLFRHIPFLAPIMADLVAGEFRKKQKNENKEVNSIMHDSSLTDCLKALGQIALCLNY |
| SEQ ID NO: | SRNFYTHANPYNSESDQEQQFDIQKTIACYLDKAFVASRRIAKKRNGYSEKDLKFLTDKAPANEDSEKYRM |
| 4648 | EEVFVLDENNQKIKKVEKDDNGKIKLDKKGDPIYIYKKKVIXXXXXXXXXXXXXXXXXXXXXXXXXXX |
| | XXXXXXXXXXXXXXXXXXXXXXXXXXXXXXXXXXXXXXXXXXVWQLQSITS |
| IMG_ | MANFHFKDRHVFGTYLNMAHTNFYRTILYVFSASGIDCYTLKGDLYVTERNVSKVTDAFCRIRNNENSEEA |
| 3300031760 | ISLLLDIVKRCYDERWNVEKDALTGLNDHQKKNKEDEFYAQCSDEGATGEKAHLLTAFTLRSEQEERLRK |
| SEQ ID NO: | LLFRHIPFLAPIMADLVAGEFRKKQKNENNEVNSIMHDSSLTDCLKALGQIALCLNYSRNFYTHANPYNSES |
| 4649 | DQEQQFDIQKTVACYLDKAFVASRRIAKKRNGYSEKDLKFLTDKAPANEDSEKYRMEEVFVLDENNQKIK |
| | KVEKDDNGKIKLDKKGDPIYIYKKKVTKDDKGNPILNEKGKKQYEIVRDDKGNPIHEYETKFVERKQWYFR |
| | LFGSCKVLLPDPEVDSNKSKLGLSDFGMLYFCALFLSKEQLVQFCTEAKVFVNSPFNNDNNKKNNIILNMM |
| | YVYQTHIPRGKRLDSERDSQALAMDMLNELRRCPIELYNVLPAIGKREFEDNVIHPNSRTPELSKRIRTKDR |
| | F |
| mgm4547164.3_ | MANYQAPPRHIFGTYLNIARANFYNTILYVFSSSGIDCYTKRGDLFVREDTVDKIIGAFSQIISGENEEMAYH |
| 7 | TIKDIVSKSYDKRWKEDNTLRGNLVNSELNAKRAEFKSPLNDEGPDGEDARIRRSFTLGSEQEERLRKLLFR |
| SEQ ID NO: | HIPLLTPIMADVIAMQFKETTNEHQEANRTLHDATLADCLKELSNIARCLTDSRNFYTHKNPYDSIEAQRTK |
| 4650 | FKLQQIIASNLDKAFIGSRRIAKKRNDYSEKDLTFLTGHDDNCRMEEVFVLDENGEKIWKVEKDKNGKDKL |
| | DKNKNSIYVYKKVKVKDKNGRNKLDEKGKPIYETLLENGEPVHEYETKFVXXXXXXXXXXXXXXXXXX |
| | XXXXXXXXXXG |
| IMG_ | LTAESIIKNKYNALNVFFEFVTSGADLNSIKKKQIDLGLADNELPDKIRCFIGTKKLFNIDGKPLLDKNTREQ |
| 3300026539 | LLKEWRPEAELRRHTINRLKAIIEEDTYRIESLGKKREKIEVGGRNNRYGRKGRADIRPGAIARYITKSLML |
| SEQ ID NO: | WQPAMPVAGGGKLTSANHQALAKYLEEYGSNDESLNNLHSIFQRAGLIGGTHPHPFINKVLKQKPMSILQL |
| 4651 | YTFYLEVEKAYAERMLAEVKTNTDDITLPPFAHPNGLRWSTTLTEAAKRYTTVPNEQNDASTDSHRAVMS |
| | LPDGLFTSHIISLLRQALPHNSELEPMRKILEGNNNMLGAAYIIRFWFDQVEGDAPQAFYNNDGDQYRRFY |
| | KTLSLLNPHRMKNRQLIPDYFSESEIKELLQIMKKKNRDQLRSAVMKAYGLKENKAEDIKQRQNAFLEML |
| | HDVSDSEQALHRYRIQDICLFLSGRSFLVDILTKSMGGNIEKKTLEKAKEMHLRDFGFDNEFEFLDNAKIPY |
| | VFKIGNMTISMPAMSFKNYGTIFRLLGDERLKQLLEGLSAMNIYE |
| UOOO01.1 | MLEAMNNSVSTTISKLFNKGVKQQRIWTFFNKRLYTIDTFDNLKAKILAKPLVFDRGVFDDKPTFIKNKQY |
| SEQ ID NO: | KDCPESFADWYQYTQNYTDYQAFYDYDRDYGELYELEKKNGGLTDKEKFIIRLKRDRKIKQTQIQDLFLK |
| 4652 | LIAEDLGKKVFNLSAEKSVISLSDIFTSRTERINQQVDAISQSQREKGDHSENKIKESYIWSKTFAYKQGQINE |
| | PEVKLKDFGKFRQLVEDARVKTIFSYNPSKEWTKKEIEEELQQYELIRREYVFKEIQDFEAYLWEQENTKG |
| | HPNNFEREGVPNFRKYIVEGVLGNLLRKHEPLIQESDKEWLKEVEINENNIASLSKQKTFVQKAFFLILLRN |
| | KFAHNQLIHVEYWNLLQNRYKPYSEDSFTSVADYLLQVTKNVISDLKKELEKT |
| IMG_ | MTAQRMIKEKINLFGNLSEVTKHKSDFFEKESAAQGWEFFPNPSYNWAGNNIPVYIDMIGKEGKAKEIQVQ |
| 3300028764_2 | INKYRKQLNPAPQRDKRISKKEIIEQLYKEKVVYGDPTLMLSANELMALLYELIVNGKSGAELENKIVEQIIA |
| SEQ ID NO: | RYEQIEAYDPTTQQLPTNQMPHKLQKSRKDSKVTDTDKLLKTIDKEIDEGNKKLELIACHRKEWEEAEQRK |
| 4653 | KSGKRNNPGTKSRKHLFYASEMGEEATWIANDLKRFMPKPARAEWKGTHHSELQRLLAFYSTQRLEAWQ |
| | LVESVWTANTHSFWEENFKEAFYKPEFENFYGAYIEIRTKILTTCRSILENNPEATMNNDTEKEWDKVFTLF |
| | DKRLYRTSTTDEQKKQLLTKPFAFPRGLFDDKPTFLPGSKPNENPERFAAWYSYGYTYSGKFQSFYDMPRN |
| | YSDWYKKLKEDGKLPRLDKKTEQEKILRFRMDCDLNIKHIRFQDIYVKLMVDSLYKAVFGQQPEFDLSHL |
| | YDTRSERYENNTIAQRQKSRQEGDNSDNAINENYVWNKTFAISLYNGRITESQVKLKDVGKFRRFATDPRV |
| | QTLISYDDTRTWTKLEIENELDNKADAYEPIRRTQSLKAIQQLEKNILKKNGFNGKQHPEELKHNGNPNFRK |
| | YIANGLLKHRTDIRQEELALISDTEFQEIGLERIRQTNPLIQKAYLLILLRNKFGHNQLPDQEHFRLMRSIYPY |
| | NQQESYSAYFNHVITQIIEELNT |
| IMG_ | MNRIGIKLNYNGHNRWSVPDKEINVKPDAIISTYEFLNLFLYEHLYQKKLTGLSPAEFIQDYLDRFNNFLSEF |
| 3300025308 | KAGHIRPVGDFSLEKRRGQGDEPDLTARRKSLQKELDRFVLKGKDLPDKIREYLLGYKQKSEKKQAKWIL |
| SEQ ID NO: | GGMIKETVYWRNKAEQSPEKMRSGDMAQQLARDIIFLTPPHTVKEHKQKLNSLEYDVLQYALAYFSSNRE |
| 4654 | KLYSFFKEHQLTVKGDRAHPFLYKIRLDECQGILDFFIVYMQQKEKWLGWLDRNLKSPRLNEEEFFNTYSY |
| | FIKTDTKRAIEMDYESCPNYLPRGIFNEPIAKALQK |

In some embodiments, the small Cas proteins are small Cas 13b-t. In some embodiments, the Cas 13b-t is Cas13b-t1, Cas13b-tla, Cas13b-t2, or Cas13b-t3. Examples of small Cas13b-t are shown in Table 3 below.

TABLE 3

| Accession | |
|---|---|
| No. | Sequences |
| IMG_ | MAVDYSLKQPFYQGVHKSCFTVPLNIAADNCKQKGYRNLLKEAQRSKGGLSDQSIQEAADLIEKRLSAIRN |
| 3300034521 | YFSHTYHTDSVLTFQKEDPVKKFLETAWSYAVSETQKDIAESDYTGIVPPLFEDKEGQFQITAAGVIFLMSF |
| SEQ ID NO: | FCHRSVLNRMFGSVKGLKRSDREQMGTGEKRDYQFTRKLLSFYSLRDSYAVKAEATRPFREILSYLSCVPH |
| 4655 | ESLVWLSARGKLTEKEKKAFRHFLDPTVPKEALSEESAGDGSDSERPGVRKNNKFLLFAVQFIEAWSRKEK |
| | KGLEFARYRKSRVEAPGENQDGSEKRIVRFRSEIRDTQEDWPYYLRNNHALLRLHPGENKEPVDARIGEYE |
| | LLYLVLAIFDGKGAKAIQKLANYIFEAKKQIQNARVYDRYQDLLPSFLTAGNKPVSAETIRNRLAYIRGELE |
| | KMLEAVQKEKKSGRWEMHKGKKIGHILRFLSNSIDDIRRRPNVKEYNRLRDLLQQLQWDEFDKALQSYVN |
| | EKLLDETVYRQLRGFHSLDELFERCCRLELKRLEDMEKAGGDRLNRYIGLEPKGKPKNYADLNTLQKKGE |
| | RFLKGHQLSIPRYFLRNALYKEYQATEERKPTSLYQIVRERLPRTNPILPDRYYLLEEDPKTYSGSDSKIIREM |
| | CFTYIEDLLCMRMARWHYEQLSEKLRKKLQWKEVQTGPAGYERFRLIYKISDELSIEFHPSDLTRLDVIEKD |
| | DMLTNISQHFLTKKGTVRWTEFVSQGMKHYRDRQKQGIEALFKWEESLRIPEGLWKEEGYLGFEKVLEEA |
| | VKHGKIQDKDKEALKRIRNDFFHEHFCGTPADWEVFKRVLKRFLNQGKNEKKRFKK |
| IMG_ | MAVDYSLKQPFYQGVHKSCFTVPLNIAADNCKQKGYRNLLKEAQRSKGGLSDQSIQEAADLIEKRLSAIRN |
| 3300033999 | YFSHTYHTDSVLTFQKEDPVKKFLETAWSYAVSETQKDIAESDYTGIVPPLFEDKEGQFQITAAGVIFLMSF |
| SEQ ID NO: | FCHRSVLNRMFGSVKGLKRSDREQMGTGEKRDYQFTRKLLSFYSLRDSYAVKAEATRPFREILSYLSCVPH |
| 4656 | ESLVWLSARGKLTEKEKKAFRHFLDPTVPKEALSEESAGDGSDSERPGVRKNNKFLLFAVQFIEAWSRKEK |
| | KGLEFARYRKSRVEAPGENQDGSEKRIVRFRSEIRDTQEDWPYYLRNNHALLRLHPGENKEPVDARIGEYE |
| | LLYLVLAIFDGKGAKAIQKLANYIFEAKKQIQNARVYDRYQDLLPSFLTAGNKPVSAETIRNRLAYIRGELE |
| | KMLEAVQKEKKSGRWEMHKGKKIGHILRFLSNSIDDIRRRPNVKEYNRLRDLLQQLQWDEFDKALQSYVN |
| | EKLLDETVYROLRGFHSLDELFERCCRLELKRLEDMEKAGGDRLNRYIGLEPKGKPKNYADLNTLQKKGE |
| | RFLKGHQLSIPRYFLRNALYKEYQATEERKPTSLYQIVRERLPRTNPILPDRYYLLEEDPKTYSGSDSKIIREM |
| | CFTYIEDLLCMRMARWHYEQLSEKLRKKLQWKEVQTGPAGYERFRLIYKISDELSIEFHPSDLTRLDVIEKD |
| | DMLTNISQHFLTKKGTVRWTEFVSQGMKHYRDRQKQGIEALFKWEESLRIPEGLWKEEGYLGFEKVLEEA |
| | VKHGKIQDKDKEALKRIRNDFFHEHFCGTPADWEVFKRVLKRFLNQGKNEKKRFKK |
| IMG_ | MAVDYSLKQPFYQGVHKSCFTVPLNIAADNCKQKGYRNLLKEAQRSKGGLSDQSIQEAADLIEKRLSAIRN |
| 3300033986 | YFSHTYHTDSVLTFQKEDPVKKFLETAWSYAVSETQKDIAESDYTGIVPPLFEDKEGQFQITAAGVIFLMSF |
| SEQ ID NO: | FCHRSVLNRMFGSVKGLKRSDREQMGTGEKRDYQFTRKLLSFYSLRDSYAVKAEATRPFREILSYLSCVPH |
| 4657 | ESLVWLSARGKLTEKEKKAFRHFLDPTVPKEALSEESAGDGSDSERPGVRKNNKFLLFAVQFIEAWSRKEK |
| | KGLEFARYRKSRVEAPGENQDGSEKRIVRFRSEIRDTQEDWPYYLRNNHALLRLHPGENKEPVDARIGEYE |
| | LLYLVLAIFDGKGAKAIQKLANYIFEAKKQIQNARVYDRYQDLLPSFLTAGNKPVSAETIRNRLAYIRGELE |
| | KMLEAVQKEKKSGRWEMHKGKKIGHILRFLSNSIDDIRRRPNVKEYNRLRDLLQQLQWDEFDKALQSYVN |
| | EKLLDETVYRQLRGFHSLDELFERCCRLELKRLEDMEKAGGDRLNRYIGLEPKGKPKNYADLNTLQKKGE |
| | RFLKGHQLSIPRYFLRNALYKEYQATEERKPTSLYQIVRERLPRTNPILPDRYYLLEEDPKTYSGSDSKIIREM |
| | CFTYIEDLLCMRMARWHYEQLSEKLRKKLQWKEVQTGPAGYERFRLIYKISDELSIEFHPSDLTRLDVIEKD |
| | DMLTNISQHFLTKKGTVRWTEFVSQGMKHYRDRQKQGIEALFKWEESLRIPEGLWKEEGYLGFEKVLEEA |
| | VKHGKIQDKDKEALKRIRNDFFHEHFCGTPADWEVFKRVLKRFLNQGKNEKKRFKK |
| IMG_ | MAVDYSLKNEWYREINKSCFTVALNVAYDNCKAKGHENLLREAQRSKGGITNEQIKNVQTEIKTRLEDIRS |
| 33000316512 | HFSHFYHDEKSLIFEKDNIVKDFLESAYEKAQSSVIGSTRQSDYKGVVPPLFEPHDGMITAAGVVFLASFFC |
| SEQ ID NO: | HRSNVYRMLGAVKGFKHTGKEELSDGAKRDYGFTRRLMAHYSLRDSYVIKAEETKSFRDLLGYLSRVPQ |
| 4658 | QAVDWLNEHNQLSEDEKKEFLNQKPSDEESQEQSKTENTORQADRMPRRSLRKTDKFILFAAKFIEDWAQ |
| | KEKMDVTFARYQKTVTEDENKNQDGKQVRDVQLKYEKDTKKLNPDFDYKWTYYIRNNHAIIQIKPDEYK |
| | QAVSARISENELKYLVLLIFQGKGWEAIKKIGDYIFHIGNKIKIGRFDHNEERRMPSFLKNPPADIIGEMVEN |
| | RLKYIRDELNKVIETIKKEEPQNNKWLLYKGKKISIILKFISDSISDIKKRPDVNEYNTLRDMLQKLDFDNFY |
| | ERLKSYVSEGRIEQTLYDEIKGIKDISTLCIKICELRLAALEELEKEGGDDLNKYIGLAVQEKHKNYDDSNTP |
| | QKKAERFLESQFSVGKNFLRETFYDEYIKNRKSLYEIIKEKITGITPLNENRWYLMDKNPKEFESKDSKIIRG |
| | LCNIYIQDILCMKIALWYYENLSPSYKNKLKWDFIGQGFGYDRYKLSYKTDCGITIEFKLADLNRLDIIEKPK |
| | MIENICHSFILEKDVKKQTISWHEFRQDGIAKYRKLQKEVVEAVFEFENSLKIPDKNWLTQGYVPFNKNKRF |
| | EDKGFSTFILEEAVRKGKIKSDDKEPLRKVRTDFFHEQFDSTDAERRIFDKYMPAKHDGKNKGGKMQEKQ |
| | EKSYTRRI |
| IMG_ | MAVDYSLKNEWYREINKSCFTVALNVAYDNCKAKGHENLLREAQRSKGGITNEQIKNVQTEIKTRLEDIRS |
| 3300031620 | HFSHFYHDEKSLIFEKDNIVKDFLESAYEKAQSSVIGSTRQSDYKGVVPPLFEPHDGMITAAGVVFLASFFC |
| SEQ ID NO: | HRSNVYRMLGAVKGFKHTGKEELSDGAKRDYGFTRRLMAHYSLRDSYVIKAEETKSFRDLLGYLSRVPQ |
| 4659 | QAVDWLNEHNQLSEDEKKEFLNQKPSDEESQEQSKTENTDRQADRMPRRSLRKTDKFILFAAKFIEDWAQ |
| | KEKMDVTFARYQKTVTEDENKNQDGKQVRDVQLKYEKDTKKLNPDFDYKWTYYIRNNHAIIQIKPDEYK |
| | QAVSARISENELKYLVLLIFQGKGWEAIKKIGDYIFHIGNKIKIGRFDHNEERRMPSFLKNPPADIIGEMVEN |
| | RLKYIRDELNKVTETIKKEEPQNNKWLLYKGKKISIILKFISDSISDIKKRPDVNEYNTLRDMLQKLDFDNFY |
| | ERLKSYVSEGRIEQTLYDEIKGIKDISTLCIKICELRLAALEELEKEGGDDLNKYIGLAVQEKHKNYDDSNTP |
| | QKKAERFLESQFSVGKNFLRETFYDEYIKNRKSLYEIIKEKITGITPLNENRWYLMDKNPKEFESKDSKIIRG |
| | LCNIYIQDILCMKIALWYYENLSPSYKNKLKWDFIGQGFGYDRYKLSYKTDCGITIEFKLADLNRLDIIEKPK |
| | MIENICHSFILEKDVKKQTISWHEFRQDGIAKYRKLQKEVVEAVFEFENSLKIPDKNWLTQGYVPFNKNKRF |
| | EDKGFSTFILEEAVRKGKIKSDDKEPLRKVRTDFFHEQFDSTDAERRIFDKYMPAKHDGKNKGGKMQEKQ |
| | EKSYTRRI |
| IMG_ | MAVDYSLKNEWYREINKSCFTVALNVAYDNCKAKGHENLLREAQRSKGGITNEQIKNVQTEIKTRLEDIRS |
| 3300031654 | HFSHFYHDEKSLIFEKDNIVKDFLESAYEKAQSSVIGSTRQSDYKGVVPPLFEPHDGMITAAGVVFLASFFC |
| SEQ ID NO: | HRSNVYRMLGAVKGFKHTGKEELSDGAKRDYGFTRRLMAHYSLRDSYVIKAEETKSFRDLLGYLSRVPQ |
| 4660 | QAVDWLNEHNQLSEDEKKEFLNQKPSDEESQEQSKTENTDRQADRMPRRSLRKTDKFILFAAKFIEDWAQ |
| | KEKMDVTFARYQKTVTEDENKNQDGKQVRDVQLKYEKDTKKLNPDFDYKWTYYIRNNHAIIQIKPDEYK |
| | QAVSARISENELKYLVLLIFQGKGWEAIKKIGDYIFHIGNKIKIGRFDHNEERRMPSFLKNPPADIIGEMVEN |
| | RLKYIRDELNKVTETIKKEEPQNNKWLLYKGKKISIILKFISDSISDIKKRPDVNEYNTLRDMLQKLDFDNFY |
| | ERLKSYVSEGRIEQTLYDEIKGIKDISTLCIKICELRLAALEELEKEGGDDLNKYIGLAVQEKHKNYDDSNTP |
| | QKKAERFLESQFSVGKNFLRETFYDEYIKNRKSLYEIIKEKITGITPLNENRWYLMDKNPKEFESKDSKIIRG |
| | LCNIYIQDILCMKIALWYYENLSPSYKNKLKWDFIGQGFGYDRYKLSYKTDCGITIEFKLADLNRLDIIEKPK |
| | MIENICHSFILEKDVKKQTISWHEFRQDGIAKYRKLQKEVVEAVFEFENSLKIPDKNWLTQGYVPFNKNKRF |
| | EDKGFSTFILEEAVRKGKIKSDDKEPLRKVRTDFFHEQFDSTDAERRIFDKYMPAKHDGKNKGGKMQEKQ |
| | EKSYTRRI |
| IMG_ | MAVDYSLKNEWYREINKSCFTVALNVAYDNCKAKGHENLLREAQRSKGGITNEQIKNVQTEIKTRLEDIRS |
| 3300031575_2 | HFSHFYHDEKSLIFEKDNIVKDFLESAYEKAQSSVIGSTRQSDYKGVVPPLFEPHDGMITAAGVVFLASFFC |
| SEQ ID NO: | HRSNVYRMLGAVKGFKHTGKEELSDGAKRDYGFTRRLMAHYSLRDSYVIKAEETKSFRDLLGYLSRVPQ |
| 4661 | QAVDWLNEHNQLSEDEKKEFLNQKPSDEESQEQSKTENTDRQADRMPRRSLRKTDKFILFAAKFIEDWAQ |
| | KEKMDVTFARYQKTVTEDENKNQDGKQVRDVQLKYEKDTKKLNPDFDYKWTYYIRNNHAIIQIKPDEYK |
| | QAVSARISENELKYLVLLIFQGKGWEAIKKIGDYIFHIGNKIKIGRFDHNEERRMPSFLKNPPADIIGEMVEN |
| | RLKYIRDELNKVTETIKKEEPQNNKWLLYKGKKISIILKFISDSISDIKKRPDVNEYN |
| IMG_ | MAVNYSLNNKYYKDVEKSCFTVALNIAHDNCMVKGHENLLREAQRSKGGITDEMILNVQNQIESFLKNM |
| 3300031624_3 | RNYFSHYYHSDKCLIFEKDDPVKVFLESVYETTKSSVIGGTRQSDYKGVTPPIFEPHNGNYMITAAGVIFLAS |
| SEQ ID NO: | FFCHRSNVYRMLGAVKGFKHTGKEELSDGQKRDYGFTRKLLAHYSLRDSYSIKAEETKSFREVLGYLSLVP |
| 4662 | QKAVDWLNERNELSKDEKEEFLKQQTCEKKEDPQEQSKSENEDKRTDKIPKRSLCKTNKFILFAIKFIEELA |
| | QKEKLDVSFARYQKKVTEAENKNQDGKQARVVQLKYKRERGKQIKNPDFDRQWTYYIREEHAIIQIKPKD |
| | KQTVSARISENELKYLVLLIFEDKGREAFHELADYIFRISKSIMHNNYKPEDARRIPSFLKKSSQTVTNKMIQ |
| | NRLKYIRYDLEKVKKTIDKEDPQNDKWLIYKGTKISIILKFISGSIADISKRPNVKEYDFLRDVLQKLDFKSFY |
| | ERLKNYVSDGRIERKLYEKIKGIEDISELCKKVCELTLERLKSLEEKGGSELNRYIGLEAQEKHKEYEDWNK |
| | PQKKAGRFLDSQFSIGKNFLRETFYDEYIRDRKSLYEIIKEKLKDILPLNEYRWYLMDRDPREYERKEGKLIR |
| | QICNTFIQDVLCMKMALWYYENLSPSYKDKLKWDSSGQGFGYDRYKLSYRTNCGVTIEFKLADLTRLDIIE |
| | KPTMIENICRSFIVKKKDSNDKIIS |
| IMG_ | MGIDYSLTSDCYRGINKSCFAVALNIAYDNCDHKGCRTLLSEVLRSKGGISDEQIKSQVVDGIQKRLKDIRN |
| 3300025161 | YFSHYYHAEDCLRFGDQDAVKVFLEEIYKNAESKTVGATKESDYKGVVPPLFELHNGTYMITAAGVIFLAS |
| SEQ ID NO: | FFCHRSNVYRMLGAVKGFKHTGKEQLSDGQKRDYGFTRRLLAYYALRDSYSVGAEDKTRCFREILSYLSR |
| 4663 | VPQLAVDWLNEQQLLTPEEKEAFLNQPAEDEGGDISDSSSSDKNKKSKEKRRSLRRDEKFILFAIQFIEGWA |
| | AEQGLDVTFARYQKTVEKAENKNQDGKQARAVQLKYRNQGLNPDFNNEWMYYIQNEHAIIQIKLNNKKA |
| | VAARISENELKYLVLLIFEEKGNDAVQKLNCYIYSMSQKIEGEWKHRPEDERWMPSFTKRADRTVTPEAVQ |
| | SRLSYIRKQLQETIEKIGQEEPRNNKWLIYKGKKISMILKFISDSIRDIQRRPNVKQYHILRDALQRLDFDGFY |
| | KELQNYVNDGRIAVSLYDQIKGVNDISGLCKKVCELTLERLAGLEAKNGSELRRYIGLEAQEKHPKYGEW |
| | NTLQEKAKRFLESQFSIGKNFLRKMFYGDCCQKRCFDEEKGYNTQAKERKSLYSIVKEKLKDIKPIHDDRW |
| | YLIDRNPKNYDNKHSRIIRQMCNTYIQDVLCMKMAMWHYEKLISATEFRNKLEWNCIGQGNMGYERYSL |
| | WYKTGCGVVIQFTPADFLRLDIIEKPAMIENICQCFVLGNKKLNSGAEKKITWDKFNKDGIAKYRKRQAEA |
| | VRAIFAFEEGLKIQEDKWSHERYFPFCNILDEAVKQGKIKDTGKDKEALNRGRNDFFHEEFKSTEDQQAIFQ |
| | KYFPIVERKDDTKKRRDKKQK |
| IMG_ | MGIDYSLTSDCYRGINKSCFAVALNIAYDNCDHKGCRTLLSEVLRSKGGISDEQIKSQVVDGIQKRLKDIRN |
| 3300007072 | YFSHYYHAEDCLRFGDQDAVKVFLEEIYKNAESKTVGATKESDYKGVVPPLFELHNGTYMITAAGVIFLAS |
| SEQ ID NO: | FFCHRSNVYRMLGAVKGFKHTGKEQLSDGQKRDYGFTRRLLAYYALRDSYSVGAEDKTRCFREILSYLSR |
| 4664 | VPQLAVDWLNEQQLLTPEEKEAFLNQPAEDEGGDISDSSSSDKNKKSKEKRRSLRRDEKFILFAIQFIEGWA |
| | AEQGLDVTFARYQKTVEKAENKNQDGKQARAVQLKYRNQGLNPDFNNEWMYYIQNEHAIIQIKLNNKKA |
| | VAARISENELKYLVLLIFEEKGNDAVQKLNCYIYSMSQKIEGEWKHRPEDERWMPSFTKRADRTVTPEAVQ |
| | SRLSYIRKQLQETIEKIGQEEPRNNKWLIYKGKKISMILKFISDSIRDIQRRPNVKQYHILRDALQRLDFDGFY |
| | KELQNYVNDGRIAVSLYDQIKGVNDISGLCKKVCELTLERLAGLEAKNGSELRRYIGLEAQEKHPKYGEW |
| | NTLQEKAKRFLESQFSIGKNFLRKMFYGDCCQKRCFDEEKGYNTQAKERKSLYSIVKEKLKDIKPIHDDRW |
| | YLIDRNPKNYDNKHSRIIRQMCNTYIQDVLCMKMAMWHYEKLISATEFRNKLEWNCIGQGNMGYERYSL |
| | WYKTGCGVVIQFTPADFLRLDIIEKPAMIENICQCFVLGNKKLNSGAEKKITWDKFNKDGIAKYRKRQAEA |
| | VRAIFAFEEGLKIQEDKWSHERYFPFCNILDEAVKQGKIKDTGKDKEALNRGRNDFFHEEFKSTEDQQAIFQ |
| | KYFPIVERKDDTKKRRDKKQK |
| IMG_ | MDNKKKGNNYSIENYKEDRFLFTAALNIAYDNCKQKGCLNILAECQHSKGGISDEQIKNVKDGIESRLRDI |
| 3300028603 | RNYFSHYYHNENCLMFEKDDPIKVFMEATFDKAVSNLSGSTKESDYKGIEPEQLRLFEEYDKKYRITMPGV |
| SEQ ID NO: | VFLASFFCHRSNVNRMMGAIKGLKRADRAEMDDGTKRDYNFTRRLLSYYSLRDSYAVKNEETRPFREILG |
| 4665 | YLSLVPHEAVDWLDSRGELSNEEKKEFLKEAKNQESKEDNDSTDEKTRRGLRKGNKFMRFAIMFTEDWSK |
| | KENLEVTFARYEKQEVHLENKKQDGKKERNIKFPHEISASDDDWPYYIRNNHAIIRIKLKDKDAVSARISEN |
| | ELKYLVLLIFENKGKEAIQKLGDYIFDMSQKIRYDNYEPKDARRIPSFLKITRKEPTYEEVNNRLTHIRRELG |
| | KIIETIEKELKESKWLIYKGKKITIILKFLSSSIADIKKRFNVEQHDALRDMLQKLKFDEFYKRLSSYVGDGTL |
| | DKKTYESIQGIKDISQLCKKACELRLARLDELEKNGGSVLYRYIGLEAEEKNKEYEKLNTNQAKAERFLES |
| | QFSTGKDFLRESFYEQEREQKKSLIKIVKEQFANVVPMNEERWYLMNKNPKKFKDKDNKAIKALCNTYVQ |
| | DILCMKIARWYYEGLSHAYKDKIEWDSTVETGGCGYTRFRLNYKTDCGVVIEFKPSDFTRLDIIEKPKMVE |
| | NICRSFITSNNDKKRTISWYDFNKEGVTKYRKQQVKAIERIFAFEKGLKIQDEKWQVQGYVPFIKRPEYENK |
| | GFKTFILEDAIQQSKIAEADKETLNKVRKDYFHEQFFSSDEDRKVFEKCMPVVDDKKKFGKKNNRMYGKK |
| | G |
| IMG_ | MEKYLIKNFEGINKSKFTVALNIANDNCKNKGIQELLKEAQRSKGGITDTQITEVQEHIKERLNSVRNYFSH |
| 3300029891 | CYHEKKPLYFEANDPVKIFLEETFAKAVENLQGRFLSDKYKLTVPPLFEPNQNNTITAAGVIFLASFFCHRSY |
| SEQ ID NO: | VYRMLGGIPGFKRSDKKKWGDGQKIDYGFTRKLMSFYSLRDSYSVNVQENKELTAFRDILGYLARVPGQA |
| 4666 | IDWLIEKGKLTKEEGKQFYLGEQSEEREEKAKKEEIKYALRKTDKFMLFAVRFIEDWAEQERIKVEFARYE |
| | KMTIVNENKKQDEKEERKVKFVSDEPTAAGWTYYIRNNHAIIKIIPDDKKKKAVSARISENELKYLVLTIID |
| | GNGKNAIAYIGDYIFRTARQIENKSYNAESEKYAPAFVRGGQKKSVDKRIKYIRDEIQQVINDIEAEQEKQK |
| | NEQDAPAENRTWLIYKGKKISIILRYVNDNIAEYKKRLSVTEYNELRGYLQQLDFINFHRKLAEYQHHGRLP |
| | NGFAESINKFQDLSKLCIEVCERQKKKLQEMAAKGGIELEQYIGLAPKEENQEQNKYATKANNFIKVWLSIP |
| | ENFLRQKFYDKFCKQQECKNKGSDKPDNTSVPQRKYFIAIIREKNIRPIHADKYYLLGQNPKDYERPDGKII |
| | RQLCDVYCKDGLCMAMAKWYYENRLGKFKDLIEWQTGDDKQQHGYAGHTLEYQATEKIKIRFKLADFT |
| | RLDIIEPPERVKNICRQWETELLKKTRDGTISWYDFKLNGLEPYRQWQGYAVADIFWFEESLKINETQWQG |
| | RTHMPFNFEKDKPLWCNILDEAVKQNKIEKQDTQALRRVRHDCFHEEFLANYEQLKIFKNLISDKAKDAKP |
| | KDKKSRKNEQKYGKR |
| IMG_ | MPVNYSLDQDYYKGTHKSCFTVPLNIAWDNGSKKGCENLLKEAMRTRGGFTQEDIEKVHRSLAEKLNGIR |
| 3300025629 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4667 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300025638 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4668 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300025629_2 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4669 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300009658 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4670 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300009658_2 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4671 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300025638_2 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4672 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300025613 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4673 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300025613_2 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4674 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300025686 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4675 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300025686_2 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4676 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300009714 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4677 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300009714_2 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4678 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300009655 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4679 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300009655_2 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4680 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300009704 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4681 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300009664 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4682 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300009704_2 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4683 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300009664_2 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4684 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300025689 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4685 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300025689_2 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4686 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300009667 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4687 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MPVNYSLDQDYYKGTHKSCFTVPLNIAWDNGSKKGCENLLKEAMRTRGGFTQEDIEKVHRSLAEKLNGIR |
| 3300009667_2 | DYFSHYYHEDKPLEFKKGDDDAVKDFLEKTFSYAAGETQKRVKESGYQGIIPPIFELCGDQVRITAAGVIFL |
| SEQ ID NO: | ASFFVPRSTLERMFGAVQGFKRSDRGDLDTGQKRDYYFTRSLLSFYTLRDSYYLQADETRPFREILSYLSCV |
| 4688 | PFDSVQWLQAHGKLSKSEEKEFFGRPVEEQDEENPAQTEKQTAPAGRRMRKKNKFILFAVRFIEAWARNE |
| | KLSVEFGRYRNIQNEEDRRKQSGKKVREVFFPSALNNLSAEEQDLEGLLYIRNNHALIRIHLKAKTPVTVRIS |
| | EHELMYLVLAILSGKGGNAVQKLSKYVWDVRMRSRGPLTNMPRNFPAFLRSPASEVSEQAVQNRLNYIRK |
| | TLKEIQANLQKEAQTGQWILDKGQKIRHILRFISDSMPDFRRRPSVKEYNELRELLQTLAFDDFYRKLASFQ |
| | TERKLDAAVWNNLAQCKSINELCERCCQLQQQRLDELEKQGGDELKRYIGLLPKEKGKHYEEQNTPARKF |
| | ERFIENQLSVPKYFLRCKLFVTGGSRRTNLLKLVQEHLKPKTSVFHEERLYLREEQPGDYPWSDRKIIQKMY |
| | YLYVQDLLCMQMAQWHYEHLTPQVKGKIDWEINSESKESDGYNRFKVEYKGPQGCRIIFRVQDFGRLDFL |
| | NKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| IMG_ | MKVENIKEKSKKAMYLINHYEGPKKWCFAIVLNRACDNYEDNPHLFSKSLLEFEKTSRKDWFDEETRELV |
| 3300031643 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4689 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031651 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4690 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031365_2 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4691 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300032029_3 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4692 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031620_2 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4693 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 33000313312 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4694 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031586_3 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4695 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVONLSGOKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031369_2 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4696 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031864 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4697 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031368 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4698 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031575 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4699 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031278_3 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4700 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031356_3 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4701 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031358_3 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4702 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031553_2 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4703 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031355 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4704 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031379 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4705 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031654_2 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4706 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031257 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4707 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031337_2 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4708 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031280 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4709 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031650_2 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4710 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300031275 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4711 | MFRDILGYLSRVPTESFQRIKQPQIRKEGQLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLRDQLPYIEGKNKEEIKEYVRFFPRFIRSHLGLLQINDEEKIKARLDYVKTKWLDKKEKS |
| | KELELHKKGRDILRYINERCDRELNRNVYNRILELLVSKDLTGFYRELEELKRTRRIDKNIVQNLSGQKTIN |
| | ALHEKVCDLVLKEIESLDTENLRKYLGLIPKEEKEVTFKEKVDRILKQPVIYKGFLRYQFFKDDKKSFVLLV |
| | EDALKEKGGGCDVPLGKEYYKIVSLDKYDKENKTLCETLAMDRLCLMMARQYYLSLNAKLAQEAQQIE |
| | WKKEDSIELIIFTLKNPDQSKQSFSIRFSVRDFTKLYVTDDPEFLARLCSYFFPVEKEIEYHKLYSEGINKYTN |
| | LQKEGIEAILELEKKLIERNRIQSAKNYLSFNEIMNKSGYNKDEQDDLKKVRNSLLHYKLIFEKEHLKKFYE |
| | VMRGEGIEKKWSLIV |
| IMG_ | MKVENIKEKSKKAMYLINHYEGPKKWCFAIVLNRACDNYEDNPHLFSKSLLEFEKTSRKDWFDEETRELV |
| 3300032062_3 | EQADTEIQPNPNLKPNTTANRKLKDIRNYFSHHYHKNECLYFKNDDPIRCIMEAAYEKSKIYIKGKQIEQSDI |
| SEQ ID NO: | PLPELFESSGWITPAGILLLASFFVERGILHRLMGNIGGFKDNRGEYGLTHDIFTTYCLKGSYSIRAQDHDAV |
| 4712 | MFRDILGYLSRVPTESFQRIKQPQIRKEGOLSERKTDKFITFALNYLEDYGLKDLEGCKACFARSKIVREQEN |
| | VESINDKEYKPHENKKKVEIHFDQSKEDRFYINRNNVILKIQKKDGHSNIVRMGVYELKYLVLMSLVGKAK |
| | EAVEKIDNYIQDLR |
| IMG_ | MNPENIKEISKKAIYSIDQYKGAKKWCFAIVLNRACDNYEGNPHLFSESLLEFEKTNRKDWFDEETRELIEK |
| 33000316513 | ADTEIQPNPNLKPNTTANRKLKDIRNYFSHHYHKNECLYFKNDDPIRCIMEAAYEKAKIYIKGKQIEQSDIPL |
| SEQ ID NO: | PELFESSGCITPAGILLLASFFVERGILHRLMGNIGGFKDNRGEYGLTHDIFTTYCLKGSYSIQAQDHDAVMF |
| 4713 | RDILGYLSRVPTESFQRIKQPKIRKEGQLSERKTDKFIKFALNYLEHYGLKDLEGCKACFARSKIVREQEDVE |
| | SIDDKEYKPHENKKKVEIHFDQSKEYPFYINRNNVILKIQKKDGHYNIVRMGVYELKYLVLLSLSGKARDS |
| | VETIDQYIQGLRDQLPSYIEKKNEKEIQEYINFLPGFIRSHLGLLNTDDDKKLKARIAYVKAKWLDKKEKSK |
| | ELELHRKGRDILRYINERCDRELNRNVYNRILELLVGKDLAGFYRELEELKRTRRIDKNIVQNLSGQKTINA |
| | LHEKVCDLVLKEIESLDTENLKKYLGLIPKEEKEVTFKEKVNRILDQPVIYKGCRFHFT |
| IMG_ | MQVENIKKGSSQGMYSIEQYEGAKKWCFAIVLNRAQTNLQGNPKLFEETLTRFERIRKEDWFDQETKKLIY |
| 3300027901 | AKQEQNEVEEEIQKAADEKLRDLRNYFSHYFHTPDCLIFTQNDPVRIIMEKAYEKARFEQAKKEQEDISIEF |
| SEQ ID NO: | GELFEENGRITSAGVVFFASFFAERRFLNRLMGYVQGFTRTEGEYKITRDVFSTYCLRDSYSVKTPDHDAV |
| 4714 | MFRDILGYLSRVPSESYQRIKESQMRSETQLSERKTDKFILFALNYLEDYGLEDLADYTACFARTRIKREQD |
| | ENTOGKEQKPHRKKPRVEIHFERAEGDPFYIKHNNVILRTQKKGAQTYIFRMGVYELKYLVLLSLLGKGAE |
| | AVKRIDRYVHSLRNQLPHIEKKSTEEIEGYVRFLPRFVRSHLGLLGVDDEKKIKARVDYVKAKWLEKKEKS |
| | RELQLHRKGRDILRYINERCERPLNIDEYNRILELLVTKHLDGFYRELEELKKTRRIDKNIVCNLSRHKSVNA |
| | LHEKVCDLVVQELESLGREELKEYVGLIPKEEKEVSFEEKTDRVVKQPVIYKGFLRNEFFRESRKSFARLVE |
| | EAVREKGEVYDVPLGGEYYEIVSLDTFDKDNKRLYETLAMDRLLLMIARQYHLSLNKELAKRAQQIEWKK |
| | EDGEEVIIFTLKNPAQPEQSCSVRFSLRDYTKLYVMDDAEFLARLCDYFLPKDEEQIDYHRLYTQGMNRYT |
| | NLQREGIEAILELEKKTIGPEQPRPPKNYIPFSEIMDKSAYNEDDQKALRRVRNALLHHNLNFARADFKRFC |
| | GIMKREGIEKRWSLAV |
| IMG_ | MQVENIKKGSSQGMYSIEQYEGAKKWCFAIVLNRAQTNLQGNPKLFEETLTRFERIRKEDWFDQETKKLIY |
| 3300002053 | AKQEQNEVEEEIQKAADEKLRDLRNYFSHYFHTPDCLIFTQNDPVRIIMEKAYEKARFEQAKKEQEDISIEF |
| SEQ ID NO: | GELFEENGRITSAGVVFFASFFAERRFLNRLMGYVQGFTRTEGEYKITRDVFSTYCLRDSYSVKTPDHDAV |
| 4715 | MFRDILGYLSRVPSESYQRIKESQMRSETQLSERKTDKFILFALNYLEDYGLEDLADYTACFARTRIKREQD |
| | ENTOGKEQKPHRKKPRVEIHFERAEGDPFYIKHNNVILRTQKKGAQTYIFRMGVYELKYLVLLSLLGKGAE |
| | AVKRIDRYVHSLRNQLPHIEKKSTEEIEGYVRFLPRFVRSHLGLLGVDDEKKIKARVDYVKAKWLEKKEKS |
| | RELQLHRKGRDILRYINERCERPLNIDEYNRILELLVTKHLDGFYRELEELKKTRRIDKNIVCNLSRHKSVNA |
| | LHEKVCDLVVQELESLGREELKEYVGLIPKEEKEVSFEEKTDRVVKQPVIYKGFLRNEFFRESRKSFARLVE |
| | EAVREKGEVYDVPLGGEYYEIVSLDTFDKDNKRLYETLAMDRLLLMIARQYHLSLNKELAKRAQQIEWKK |
| | EDGEEVIIFTLKNPAQPEQSCSVRFSLRDYTKLYVMDDAEFLARLCDYFLPKDEEQIDYHRLYTQGMNRYT |
| | NLQREGIEAILELEKKTIGPEQPRPPKNYIPFSEIMDKSAYNEDDQKALRRVRNALLHHNLNFARADFKRFC |
| | GIMKREGIEKRWSLAV |
| IMG_ | MQVENIKKGSSQGMYSIEQYEGAKKWCFAIVLNRAQTNLQGNPKLFEETLTRFERIRKEDWFDQETKKLIY |
| 3300002052 | AKQEQNEVEEEIQKAADEKLRDLRNYFSHYFHTPDCLIFTQNDPVRIIMEKAYEKARFEQAKKEQEDISIEF |
| SEQ ID NO: | GELFEENGRITSAGVVFFASFFAERRFLNRLMGYVQGFTRTEGEYKITRDVFSTYCLRDSYSVKTPDHDAV |
| 4716 | MFRDILGYLSRVPSESYQRIKESQMRSETQLSERKTDKFILFALNYLEDYGLEDLADYTACFARTRIKREQD |
| | ENTOGKEQKPHRKKPRVEIHFERAEGDPFYIKHNNVILRTQKKGAQTYIFRMGVYELKYLVLLSLLGKGAE |
| | AVKRIDRYVHSLRNQLPHIEKKSTEEIEGYVRFLPRFVRSHLGLLGVDDEKKIKARVDYVKAKWLEKKEKS |
| | RELQLHRKGRDILRYINERCERPLNIDEYNRILELLVTKHLDGFYRELEELKKTRRIDKNIVCNLSRHKSVNA |
| | LHEKVCDLVVQELESLGREELKEYVGLIPKEEKEVSFEEKTDRVVKQPVIYKGFLRNEFFRESRKSFARLVE |
| | EAVREKGEVYDVPLGGEYYEIVSLDTFDKDNKRLYETLAMDRLLLMIARQYHLSLNKELAKRAQQIEWKK |
| | EDGEEVIIFTLKNPAQPEQSCSVRFSLRDYTKLYVMDDAEFLARLCDYFLPKDEEQIDYHRLYTQGMNRYT |
| | NLQREGIEAILELEKKTIGPEQPRPPKNYIPFSEIMDKSAYNEDDQKALRRVRNALLHHNLNFARADFKRFC |
| | GIMKREGIEKRWSLAV |
| IMG_ | MQVENIKKGSSQGMYSIEQYEGAKKWCFAIVLNRAQTNLQGNPKLFEETLTRFERIRKEDWFDQETKKLIY |
| 3300027888 | AKQEQNEVEEEIQKAADEKLRDLRNYFSHYFHTPDCLIFTQNDPVRIIMEKAYEKARFEQAKKEQEDISIEF |
| SEQ ID NO: | GELFEENGRITSAGVVFFASFFAERRFLNRLMGYVQGFTRTEGEYKITRDVFSTYCLRDSYSVKTPDHDAV |
| 4717 | MFRDILGYLSRVPSESYQRIKESQMRSETQLSERKTDKFILFALNYLEDYGLEDLADYTACFARTRIKREQD |
| | ENTOGKEQKPHRKKPRVEIHFERAEGDPFYIKHNNVILRTQKKGAQTYIFRMGVYELKYLVLLSLLGKGAE |
| | AVKRIDRYVHSLRNQLPHIEKKSTEEIEGYVRFLPRFVRSHLGLLGVDDEKKIKARVDYVKAKWLEKKEKS |
| | RELQLHRKGRDILRYINERCERPLNIDEYNRILELLVTKHLDGFYRELEELKKTRRIDKNIVCNLSRHKSVNA |
| | LHEKVCDLVVQELESLGREELKEYVGLIPKEEKEVSFEEKTDRVVKQPVIYKGFLRNEFFRESRKSFARLVE |
| | EAVREKGEVYDVPLGGEYYEIVSLDTFDKDNKRLYETLAMDRLLLMIARQYHLSLNKELAKRAQQIEWKK |
| | EDGEEVIIFTLKNPAQPEQSCSVRFSLRDYTKLYVMDDAEFLARLCDYFLPKDEEQIDYHRLYTQGMNRYT |
| | NLQREGIEAILELEKKTIGPEQPRPPKNYIPFSEIMDKSAYNEDDQKALRRVRNALLHHNLNFARADFKRFC |
| | GIMKREGIEKRWSLAV |
| IMG_ | MQVENIKKGSSQGMYSIEQYEGAKKWCFAIVLNRAQTNLQGNPKLFEETLTRFERIRKEDWFDQETKKLIY |
| 3300001752 | AKQEQNEVEEEIQKAADEKLRDLRNYFSHYFHTPDCLIFTQNDPVRIIMEKAYEKARFEQAKKEQEDISIEF |
| SEQ ID NO: | GELFEENGRITSAGVVFFASFFAERRFLNRLMGYVQGFTRTEGEYKITRDVFSTYCLRDSYSVKTPDHDAV |
| 4718 | MFRDILGYLSRVPSESYQRIKESQMRSETQLSERKTDKFILFALNYLEDYGLEDLADYTACFARTRIKREQD |
| | ENTOGKEQKPHRKKPRVEIHFERAEGDPFYIKHNNVILRTQKKGAQTYIFRMGVYELKYLVLLSLLGKGAE |
| | AVKRIDRYVHSLRNQLPHIEKKSTEEIEGYVRFLPRFVRSHLGLLGVDDEKKIKARVDYVKAKWLEKKEKS |
| | RELQLHRKGRDILRYINERCERPLNIDEYNRILELLVTKHLDGFYRELEELKKTRRIDKNIVCNLSRHKSVNA |
| | LHEKVCDLVVQELESLGREELKEYVGLIPKEEKEVSFEEKTDRVVKQPVIYKGFLRNEFFRESRKSFARLVE |
| | EAVREKGEVYDVPLGGEYYEIVSLDTFDKDNKRLYETLAMDRLLLMIARQYHLSLNKELAKRAQQIEWKK |
| | EDGEEVIIFTLKNPAQPEQSCSVRFSLRDYTKLYVMDDAEFLARLCDYFLPKDEEQIDYHRLYTQGMNRYT |
| | NLQREGIEAILELEKKTIGPEQPRPPKNYIPFSEIMDKSAYNEDDQKALRRVRNALLHHNLNFARADFKRFC |
| | GIMKREGIEKRWSLAV |
| IMG_ | MEFENIKKTSNKEVYSIEQYEGEKKWCFAIVLNRAQTNLEENPKLFEQTLTRFEKIMKQDWFNEETKKLIYE |
| 3300009529 | KEEENKVKEEIQIAASERLKNLRNYFSHYLHAPDCLIFNRNDTIRIIMEKAYEKSRFEAKKKQQEDISIEFPEL |
| SEQ ID NO: | FEEEDKITSAGVVFFVSFFIERRFLNRLMGYVQGFRKTEGEYNITRQVFSKYCLKDSYSVQAQDHDAVMFR |
| 4719 | DILGYLSRVPTEIYQHIKLTRKRSQDQLSERKTDKFILFALKYLEDYGLKDLADYTACFARSKIKRENEDTK |
| | ETDGNKHKFHREKPVVEIHFDKEKQDQFYIKRNNVILKAQKKGGQSNVFRMGVYELKYLVLLSLLGKAEE |
| | AIQRIDRYISSLKKQLPYLDKISNEEIQKSINFLPRFVRSRLGLLQVDDEKRLKTRLEYVKAKWTDKKEGSRK |
| | LELHRKGRDILRYINERCDRPLSRKEYNNILKFIVNKDFAGFYNELEELKRTRRLDKNIIQKLSGHTTLNALH |
| | ERVCDLVLQELGSLQSENLKEYIGLIPKEEKEVTFREKVDRILEQPVVYKGFLRYEFFKEDKKSFARLVEEAI |
| | KTKWSDFDIPLGEEYYNIPSLDRFDRTNKKLYETLAMDRLCLMMARQYYLRLNEKLAEKAQHIYWKKED |
| | GREVIIFKFQNPKEQKKSFSIRFSILDYTKMYVMDDPEFLSRLWEYFIPKEAKEIDYHKHYARAFDKYTNLQ |
| | KEGIDAILKLEGRIIERRKIKPAKNYIEFQEIMNRSGYNNDQQVALKRVRNALLHYNLNFEREHLKRFYGVV |
| | KREGIEKKWSLIV |
| IMG_ | MEFENIKKTSNKEVYSIEQYEGEKKWCFAIVLNRAQTNLEENPKLFEQTLTRFEKIMKQDWFNEETKKLIYE |
| 3300024433 | KEEENKVKEEIQIAASERLKNLRNYFSHYLHAPDCLIFNRNDTIRIIMEKAYEKSRFEAKKKQQEDISIEFPEL |
| SEQ ID NO: | FEEEDKITSAGVVFFVSFFIERRFLNRLMGYVQGFRKTEGEYNITRQVFSKYCLKDSYSVQAQDHDAVMFR |
| 4720 | DILGYLSRVPTEIYQHIKLTRKRSQDQLSERKTDKFILFALKYLEDYGLKDLADYTACFARSKIKRENEDTK |
| | ETDGNKHKFHREKPVVEIHFDKEKQDQFYIKRNNVILKAQKKGGQSNVFRMGVYELKYLVLLSLLGKAEE |
| | AIQRIDRYISSLKKQLPYLDKISNEEIQKSINFLPRFVRSRLGLLQVDDEKRLKTRLEYVKAKWTDKKEGSRK |
| | LELHRKGRDILRYINERCDRPLSRKEYNNILKFIVNKDFAGFYNELEELKRTRRLDKNIIQKLSGHTTLNALH |
| | ERVCDLVLQELGSLQSENLKEYIGLIPKEEKEVTFREKVDRILEQPVVYKGFLRYEFFKEDKKSFARLVEEAI |
| | KTKWSDFDIPLGEEYYNIPSLDRFDRTNKKLYETLAMDRLCLMMARQYYLRLNEKLAEKAQHIYWKKED |
| | GREVIIFKFQNPKEQKKSFSIRFSILDYTKMYVMDDPEFLSRLWEYFIPKEAKEIDYHKHYARAFDKYTNLQ |
| | KEGIDAILKLEGRIIERRKIKPAKNYIEFQEIMNRSGYNNDQQVALKRVRNALLHYNLNFEREHLKRFYGVV |
| | KREGIEKKWSLIV |
| IMG_ | MQFENIKDTGQKPIYSIDQYEGAKKWCFAIVLNRACDNYEDNPQLFSESLLRFEEVNRRDWFDKDIRDLIK |
| 3300031885 | KADTEDQIEPKRKPNTPVNRRLHDIRNYFSHSRHQDDCLYFKNDDPMRCIMEAAYEKAKIHIKGRQTEQSD |
| SEQ ID NO: | IPLPELFDANNKITSAGVLFLASFFVERGILHRLMGNIGGFKDNRGKYGLTHDIFTTYCLKDSYSIHASDPKV |
| 4721 | VLFRDIAGYLSLVACEYYPTYLSKIPKENAGEKSSDEEKYAERKTDKFILFALKYLEEFVLPSLKDDYLVDIG |
| | RIDIIREESKETEEKDEQYKPHPNQGKVKVVFDSINKELPYYINHNTVILRIQKNGVMAYSCKIGVNDLKYL |
| | LLLCLQGKTDKALDAIYNYLHSMQDPPEVVKIGATDKLFQGLPEFILKQSGIKVQDKNKEKAARIKYIRDK |
| | WEKKKSESADMELHRKGRDILRYVNWHCETPLGTEKYDQLLVLLVNKNFVVFGDELNQLKRTEIISKDILE |
| | KLSGFQTINTLHQKVCNLVLEELSSLEKNDPGKLAEHIGLVRKPAPENNPPPEYKEKVRRFVEQPMIYKGFL |
| | RDQFFVNKDQDGKKLKEQKTFAKLVEETLGQNADVPLGKDFYYVPNIEKDEKKNRFHKDNAVLYETLAL |
| | DRLCAMMARKCLTQINKNLAEKSEEIDWRNEDGKDFIYLKLVKSDRPQETFKIRFKVNDFAKLYVMDDPD |
| | FLGGLMKHFFPQEHSIEYHKLYRNGIERYTDRQKDGIEAILRLEDSVIRQKGMKPKPAKNYISFSEIMAQTD |
| | YPEHDQKVLNKVRRAVLHYHLKFEPADYNRFVDIMKKNKFWDGERKNKESRGR |
| IMG_ | MQFENIKDTGQKPIYSIDQYEGAKKWCFAIVLNRACDNYEDNPQLFSESLLRFEEVNRRDWFDKDIRDLIK |
| 3300031952 | KADTEDQIEPKRKPNTPVNRRLHDIRNYFSHSRHQDDCLYFKNDDPMRCIMEAAYEKAKIHIKGRQTEQSD |
| SEQ ID NO: | IPLPELFDANNKITSAGVLFLASFFVERGILHRLMGNIGGFKDNRGKYGLTHDIFTTYCLKDSYSIHASDPKV |
| 4722 | VLFRDIAGYLSLVACEYYPTYLSKIPKENAGGKSSDEEKYAERKTDKFILFALKYLEEFVLPSLKDDYLVDI |
| | GRIDIIREESKETEEKDEQYKPHPNQGKVKVVFDSINKELPYYINHNTVILRIQKNGVMAYSCKIGVNDLKY |
| | LLLLCLQGKTDKALDAIYNYLHSMQDPPEVVKIGATDKLFQGLPEFILKQSGIKVQDKNKEKAARIKYIRD |
| | KWEKKKSESADIELHRKGRDILRYVNWHCETPLGTEKYDQLLVLLVNKNFAGFGDELNQLKRTEIISKDIF |
| | EKLSGFKTINTLHQKVCNLVLEELSFFEKSNPEKLEEYIGLIRKPAPENNPPPEYKEKVRRFVEQPMIYKGFL |
| | RDQFFVNKDQDGKKLKEQKTFAKLVEETLGQNADVPLGKDFYYVPNIEKDEKKNRFHKDNAVLYETLAL |
| | DRLCAMMARKCLTQINKNLAEKSEEIDWRNEDGKDFIYLKLVKSDRPQETFKIRFKVNDFAKLYVMDDPD |
| | FLGGLMKHFFPQEHSIEYHKLYRNGIERYTDRQKDGIEAILRLEDSVIRQKGMKPKPAKNYISFSEIMAQTD |
| | YPEHDQKVLNKVRRALLHYHLKFEPADYNRFVDIMKKDKFWDGERKNEESRGK |
| GCA_ | MAQVSKQTSKKRELSIDEYQGARKWCFTIAFNKALVNRDKNDGLFVESLLRHEKYSKHDWYDEDTRALIK |
| 003644175.1_ | CSTQAANAKAEALRNYFSHYRHSPGCLTFTAEDELRTIMERAYERAIFECRRRETEVIIEFPSLFEGDRITTA |
| ASM364417v1_ | GVVFFVSFFVERRVLDRLYGAVSGLKKNEGQYKLTRKALSMYCLKDSRFTKAWDKRVLLFRDILAQLGRI |
| genomic | PAEAYEYYHGEQGDKKRANDNEGTNPKRHKDKFIEFALHYLEAQHSEICFGRRHIVREEAGAGDEHKKHR |
| SEQ ID NO: | TKGKVVVDFSKKDEDQSYYISKNNVIVRIDKNAGPRSYRMGLNELKYLVLLSLQGKGDDAIAKLYRYRQH |
| 4723 | VENILDVVKVTDKDNHVFLPRFVLEQHGIGRKAFKQRIDGRVKHVRGVWEKKKAATNEMTLHEKARDIL |
| | QYVNENCTRSFNPGEYNRLLVCLVGKDVENFQAGLKRLQLAERIDGRVYSIFAQTSTINEMHQVVCDQILN |
| | RLCRIGDQKLYDYVGLGKKDEIDYKQKVAWFKEHISIRRGFLRKKFWYDSKKGFAKLVEEHLESGGGQRD |
| | VGLDKKYYHIDAIGRFEGANPALYETLARDRLCLMMAQYFLGSVRKELGNKIVWSNDSIELPVEGSVGNE |
| | KSIVFSVSDYGKLYVLDDAEFLGRICEYFMPHEKGKIRYHTVYEKGFRAYNDLQKKCVEAVLAFEEKVVK |
| | AKKMSEKEGAHYIDFREILAQTMCKEAEKTAVNKVRRAFFHHHLKFVIDEFGLFSDVMKKYGIEKEWKFP |
| | VK |
| IMG_ | MAQVSKQTSKKRELSIDEYQGARKWCFTIAFNKALVNRDKNDGLFVESLLRHEKYSKHDWYDEDTRALIK |
| 3300014911 | CSTQAANAKAEALRNYFSHYRHSPGCLTFTAEDELRTIMERAYERAIFECRRRETEVIIEFPSLFEGDRITTA |
| SEQ ID NO: | GVVFFVSFFVERRVLDRLYGAVSGLKKNEGQYKLTRKALSMYCLKDSRFTKAWDKRVLLFRDILAQLGRI |
| 4724 | PAEAYEYYHGEQGDKKRANDNEGTNPKRHKDKFIEFALHYLEAQHSEICFGRRHIVREEAGAGDEHKKHR |
| | TKGKVVVDFSKKDEDQSYYISKNNVIVRIDKNAGPRSYRMGLNELKYLVLLSLQGKGDDAIAKLYRYRQH |
| | VENILDVVKVTDKDNHVFLPRFVLEQHGIGRKAFKQRIDGRVKHVRGVWEKKKAATNEMTLHEKARDIL |
| | QYVNENCTRSFNPGEYNRLLVCLVGKDVENFQAGLKRLQLAERIDGRVYSIFAQTSTINEMHQVVCDQILN |
| | RLCRIGDQKLYDYVGLGKKDEIDYKQKVAWFKEHISIRRGFLRKKFWYDSKKGFAKLVEEHLESGGGQRD |
| | VGLDKKYYHIDAIGRFEGANPALYETLARDRLCLMMAQYFLGSVRKELGNKIVWSNDSIELPVEGSVGNE |
| | KSIVFSVSDYGKLYVLDDAEFLGRICEYFMPHEKGKIRYHTVYEKGFRAYNDLQKKCVEAVLAFEEKVVK |
| | AKKMSEKEGAHYIDFREILAQTMCKEAEKTAVNKVRRAFFHHHLKFVIDEFGLFSDVMKKYGIEKEWKFP |
| | VK |
| IMG_ | MQTATQEQKQKQSIYSILNYQGQRKWCFAIVLNRALDNINPKRETETGKYKNKELFYKSLLRFEGIKKQPW |
| 3300031698 | FDETKAEKENVTAKEIIDSKDKAAELLLNLRNYFSHNYHTEKCLYFGTESQHKQIRLIMEAAYERAKAELT |
| SEQ ID NO: | GRRTGQEISAEAEKDKDGNIKKYKLSDVPWPPLFDEKDIITTAGVVFFASFFTEAGQIFRLMNWINGLKRND |
| 4725 | DKFNITRRALSFYSLPDSYAEAIAEYEVEEDGASRTIRYKAKIFKDILNYLRRIPSETYKLYHSGEENKISGKK |
| | EEKGEDENTPVERKTDKFAEFAMRYLEDFEGVRFARYRINTKTRENEVFFDEDELKKLIDKKGVPEQEKDK |
| | KFEDYRYYYVKNNAILKTEKGSIRIGINELKYFVLLSLDKMGQQAKEKINSFLSKFTGDNLGNREFIKANIEE |
| | LPPFILKKFDPLAEDKEKRIEKRVGASEKPLFSIDIL |
| IMG_ | MNGIELKKEEAAFYFNQAELNLKAIEDNIFDKERRKTLLNNPQILAKMENFIFNFRDVTKNAKGEIDCLLLK |
| 3300031365 | LRELRNFYSHYVHKRDVRELSKGEKPILEKYYQFAIESTGSENVKLEIIENDAWLADAGVLFFLCIFLKKSQ |
| SEQ ID NO: | ANKLISGISGFKRNDDTGQPRRNLFTYFSIREGYKVVPEMQKHFLLFSLVNHLSNQDDYIEKAHQPYDIGEG |
| 4726 | LFFHRIASTFLNISGILRNMKFYTYQSKRLVEQRGELKREKDIFAWEEPFQGNSYFEINGHKGVIGEDELKEL |
| | CYAFLIGNQDANKVEGRITQFLEKFRNANSVQQVKDDEMLKPEYFPANYFAESGVGRIKDRVLNRLNKAI |
| | KSNKAKKGEIIAYDKMREVMAFINNSLPVDEKLKPKDYKRYLGMVRFWDREKDNIKREFETKEWSKYLPS |
| | NFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGWQE |
| | SEKVSKKIREAECEILLSKEYEELSKQFFQSKDYDKMTRINGLYEKNKLIALMAVYLMGQLRILFKEHTKLD |
| | DITKTTVDFKISDKVTVKIPFSNYPSLVYTMSSKYVDNIGNYGFSNKDKDKPILGKIDVIEKQRMEFIKEVLG |
| | FEKYLFDDKIIDKSKFADTATHISFAEIVEELVEKGWDKDRLTKLKDARNKALHGEILTGTSFDETKSLINEL |
| | KK |
| IMG_ | MNGIELKKEEAAFYFNQAELNLKAIEDNIFDKERRKTLLNNPQILAKMENFIFNFRDVTKNAKGEIDCLLLK |
| 3300032029_2 | LRELRNFYSHYVHKRDVRELSKGEKPILEKYYQFAIESTGSENVKLEIIENDAWLADAGVLFFLCIFLKKSQ |
| SEQ ID NO: | ANKLISGISGFKRNDDTGQPRRNLFTYFSIREGYKVVPEMQKHFLLFSLVNHLSNQDDYIEKAHQPYDIGEG |
| 4727 | LFFHRIASTFLNISGILRNMKFYTYQSKRLVEQRGELKREKDIFAWEEPFQGNSYFEINGHKGVIGEDELKEL |
| | CYAFLIGNQDANKVEGRITQFLEKFRNANSVQQVKDDEMLKPEYFPANYFAESGVGRIKDRVLNRLNKAI |
| | KSNKAKKGEIIAYDKMREVMAFINNSLPVDEKLKPKDYKRYLGMVRFWDREKDNIKREFETKEWSKYLPS |
| | NFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGWQE |
| | SEKVSKKIREAECEILLSKEYEELSKQFFQSKDYDKMTRINGLYEKNKLIALMAVYLMGQLRILFKEHTKLD |
| | DITKTTVDFKISDKVTVKIPFSNYPSLVYTMSSKYVDNIGNYGFSNKDKDKPILGKIDVIEKQRMEFIKEVLG |
| | FEKYLFDDKIIDKSKFADTATHISFAEIVEELVEKGWDKDRLTKLKDARNKALHGEILTGTSFDETKSLINEL |
| | KK |
| IMG_ | MNGIELKKEEAAFYFNQAELNLKAIEDNIFDKERRKTLLNNPQILAKMENFIFNFRDVTKNAKGEIDCLLLK |
| 33000313313 | LRELRNFYSHYVHKRDVRELSKGEKPILEKYYQFAIESTGSENVKLEIIENDAWLADAGVLFFLCIFLKKSQ |
| SEQ ID NO: | ANKLISGISGFKRNDDTGQPRRNLFTYFSIREGYKVVPEMQKHFLLFSLVNHLSNQDDYIEKAHQPYDIGEG |
| 4728 | LFFHRIASTFLNISGILRNMKFYTYQSKRLVEQRGELKREKDIFAWEEPFQGNSYFEINGHKGVIGEDELKEL |
| | CYAFLIGNQDANKVEGRITQFLEKFRNANSVQQVKDDEMLKPEYFPANYFAESGVGRIKDRVLNRLNKAI |
| | KSNKAKKGEIIAYDKMREVMAFINNSLPVDEKLKPKDYKRYLGMVRFWDREKDNIKREFETKEWSKYLPS |
| | NFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGWQE |
| | SEKVSKKIREAECEILLSKEYEELSKQFFQSKDYDKMTRINGLYEKNKLIALMAVYLMGQLRILFKEHTKLD |
| | DITKTTVDFKISDKVTVKIPFSNYPSLVYTMSSKYVDNIGNYGFSNKDKDKPILGKIDVIEKQRMEFIKEVLG |
| | FEKYLFDDKIIDKSKFADTATHISFAEIVEELVEKGWDKDRLTKLKDARNKALHGEILTGTSFDETKSLINEL |
| | KK |
| IMG_ | MNGIELKKEEAAFYFNQAELNLKAIEDNIFDKERRKTLLNNPQILAKMENFIFNFRDVTKNAKGEIDCLLLK |
| 3300031369 | LRELRNFYSHYVHKRDVRELSKGEKPILEKYYQFAIESTGSENVKLEIIENDAWLADAGVLFFLCIFLKKSQ |
| SEQ ID NO: | ANKLISGISGFKRNDDTGQPRRNLFTYFSIREGYKVVPEMQKHFLLFSLVNHLSNQDDYIEKAHQPYDIGEG |
| 4729 | LFFHRIASTFLNISGILRNMKFYTYQSKRLVEQRGELKREKDIFAWEEPFQGNSYFEINGHKGVIGEDELKEL |
| | CYAFLIGNQDANKVEGRITQFLEKFRNANSVQQVKDDEMLKPEYFPANYFAESGVGRIKDRVLNRLNKAI |
| | KSNKAKKGEIIAYDKMREVMAFINNSLPVDEKLKPKDYKRYLGMVRFWDREKDNIKREFETKEWSKYLPS |
| | NFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGWQE |
| | SEKVSKKIREAECEILLSKEYEELSKQFFQSKDYDKMTRINGLYEKNKLIALMAVYLMGQLRILFKEHTKLD |
| | DITKTTVDFKISDKVTVKIPFSNYPSLVYTMSSKYVDNIGNYGFSNKDKDKPILGKIDVIEKQRMEFIKEVLG |
| | FEKYLFDDKIIDKSKFADTATHISFAEIVEELVEKGWDKDRLTKLKDARNKALHGEILTGTSFDETKSLINEL |
| | KK |
| IMG_ | MNGIELKKEEAAFYFNQAELNLKAIEDNIFDKERRKTLLNNPQILAKMENFIFNFRDVTKNAKGEIDCLLLK |
| 3300031278 | LRELRNFYSHYVHKRDVRELSKGEKPILEKYYQFAIESTGSENVKLEIIENDAWLADAGVLFFLCIFLKKSQ |
| SEQ ID NO: | ANKLISGISGFKRNDDTGQPRRNLFTYFSIREGYKVVPEMQKHFLLFSLVNHLSNQDDYIEKAHQPYDIGEG |
| 4730 | LFFHRIASTFLNISGILRNMKFYTYQSKRLVEQRGELKREKDIFAWEEPFQGNSYFEINGHKGVIGEDELKEL |
| | CYAFLIGNQDANKVEGRITQFLEKFRNANSVQQVKDDEMLKPEYFPANYFAESGVGRIKDRVLNRLNKAI |
| | KSNKAKKGEIIAYDKMREVMAFINNSLPVDEKLKPKDYKRYLGMVRFWDREKDNIKREFETKEWSKYLPS |
| | NFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGWQE |
| | SEKVSKKIREAECEILLSKEYEELSKQFFQSKDYDKMTRINGLYEKNKLIALMAVYLMGQLRILFKEHTKLD |
| | DITKTTVDFKISDKVTVKIPFSNYPSLVYTMSSKYVDNIGNYGFSNKDKDKPILGKIDVIEKQRMEFIKEVLG |
| | FEKYLFDDKIIDKSKFADTATHISFAEIVEELVEKGWDKDRLTKLKDARNKALHGEILTGTSFDETKSLINEL |
| | KK |
| IMG_ | MNGIELKKEEAAFYFNQAELNLKAIEDNIFDKERRKTLLNNPQILAKMENFIFNFRDVTKNAKGEIDCLLLK |
| 3300031356 | LRELRNFYSHYVHKRDVRELSKGEKPILEKYYQFAIESTGSENVKLEIIENDAWLADAGVLFFLCIFLKKSQ |
| SEQ ID NO: | ANKLISGISGFKRNDDTGQPRRNLFTYFSIREGYKVVPEMQKHFLLFSLVNHLSNQDDYIEKAHQPYDIGEG |
| 4731 | LFFHRIASTFLNISGILRNMKFYTYQSKRLVEQRGELKREKDIFAWEEPFQGNSYFEINGHKGVIGEDELKEL |
| | CYAFLIGNQDANKVEGRITQFLEKFRNANSVQQVKDDEMLKPEYFPANYFAESGVGRIKDRVLNRLNKAI |
| | KSNKAKKGEIIAYDKMREVMAFINNSLPVDEKLKPKDYKRYLGMVRFWDREKDNIKREFETKEWSKYLPS |
| | NFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGWQE |
| | SEKVSKKIREAECEILLSKEYEELSKQFFQSKDYDKMTRINGLYEKNKLIALMAVYLMGQLRILFKEHTKLD |
| | DITKTTVDFKISDKVTVKIPFSNYPSLVYTMSSKYVDNIGNYGFSNKDKDKPILGKIDVIEKQRMEFIKEVLG |
| | FEKYLFDDKIIDKSKFADTATHISFAEIVEELVEKGWDKDRLTKLKDARNKALHGEILTGTSFDETKSLINEL |
| | KK |
| IMG_ | MNGIELKKEEAAFYFNQAELNLKAIEDNIFDKERRKTLLNNPQILAKMENFIFNFRDVTKNAKGEIDCLLLK |
| 3300031358 | LRELRNFYSHYVHKRDVRELSKGEKPILEKYYQFAIESTGSENVKLEIIENDAWLADAGVLFFLCIFLKKSQ |
| SEQ ID NO: | ANKLISGISGFKRNDDTGQPRRNLFTYFSIREGYKVVPEMQKHFLLFSLVNHLSNQDDYIEKAHQPYDIGEG |
| 4732 | LFFHRIASTFLNISGILRNMKFYTYQSKRLVEQRGELKREKDIFAWEEPFQGNSYFEINGHKGVIGEDELKEL |
| | CYAFLIGNQDANKVEGRITQFLEKFRNANSVQQVKDDEMLKPEYFPANYFAESGVGRIKDRVLNRLNKAI |
| | KSNKAKKGEIIAYDKMREVMAFINNSLPVDEKLKPKDYKRYLGMVRFWDREKDNIKREFETKEWSKYLPS |
| | NFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGWQE |
| | SEKVSKKIREAECEILLSKEYEELSKQFFQSKDYDKMTRINGLYEKNKLIALMAVYLMGQLRILFKEHTKLD |
| | DITKTTVDFKISDKVTVKIPFSNYPSLVYTMSSKYVDNIGNYGFSNKDKDKPILGKIDVIEKQRMEFIKEVLG |
| | FEKYLFDDKIIDKSKFADTATHISFAEIVEELVEKGWDKDRLTKLKDARNKALHGEILTGTSFDETKSLINEL |
| | KK |
| IMG_ | MNGIELKKEEAAFYFNQAELNLKAIEDNIFDKERRKTLLNNPQILAKMENFIFNFRDVTKNAKGEIDCLLLK |
| 3300031624_2 | LRELRNFYSHYVHKRDVRELSKGEKPILEKYYQFAIESTGSENVKLEIIENDAWLADAGVLFFLCIFLKKSQ |
| SEQ ID NO: | ANKLISGISGFKRNDDTGQPRRNLFTYFSIREGYKVVPEMQKHFLLFSLVNHLSNQDDYIEKAHQPYDIGEG |
| 4733 | LFFHRIASTFLNISGILRNMKFYTYQSKRLVEQRGELKREKDIFAWEEPFQGNSYFEINGHKGVIGEDELKEL |
| | CYAFLIGNQDANKVEGRITQFLEKFRNANSVQQVKDDEMLKPEYFPANYFAESGVGRIKDRVLNRLNKAI |
| | KSNKAKKGEIIAYDKMREVMAFINNSLPVDEKLKPKDYKRYLGMVRFWDREKDNIKREFETKEWSKYLPS |
| | NFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGWQE |
| | SEKVSKKIREAECEILLSKEYEELSKQFFQSKDYDKMTRINGLYEKNKLIALMAVYLMGQLRILFKEHTKLD |
| | DITKTTVDFKISDKVTVKIPFSNYPSLVYTMSSKYVDNIGNYGFSNKDKDKPILGKIDVIEKQRMEFIKEVLG |
| | FEKYLFDDKIIDKSKFADTATHISFAEIVEELVEKGWDKDRLTKLKDARNKALHGEILTGTSFDETKSLINEL |
| | KK |
| IMG_ | MNGIELKKEEAAFYFNQAELNLKAIEDNIFDKERRKTLLNNPQILAKMENFIFNFRDVTKNAKGEIDCLLLK |
| 3300032062 | LRELRNFYSHYVHKRDVRELSKGEKPILEKYYQFAIESTGSENVKLEIIENDAWLADAGVLFFLCIFLKKSQ |
| SEQ ID NO: | ANKLISGISGFKRNDDTGQPRRNLFTYFSIREGYKVVPEMQKHFLLFSLVNHLSNQDDYIEKAHQPYDIGEG |
| 4734 | LFFHRIASTFLNISGILRNMKFYTYQSKRLVEQRGELKREKDIFAWEEPFQGNSYFEINGHKGVIGEDELKEL |
| | CYAFLIGNQDANKVEGRITQFLEKFRNANSVQQVKDDEMLKPEYFPANYFAESGVGRIKDRVLNRLNKAI |
| | KSNKAKKGEIIAYDKMREVMAFINNSLPVDEKLKPKDYKRYLGMVRFWDREKDNIKREFETKEWSKYLPS |
| | NFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGWQE |
| | SEKVSKKIREAECEILLSKEYEELSKQFFQSKDYDKMTRINGLYEKNKLIALMAVYLMGQLRILFKEHTKLD |
| | DITKTTVDFKISDKVTVKIPFSNYPSLVYTMSSKYVDNIGNYGFSNKDKDKPILGKIDVIEKQRMEFIKEVLG |
| | FEKYLFDDKIIDKSKFADTATHISFAEIVEELVEKGWDKDRLTKLKDARNKALHGEILTGTSFDETKSLINEL |
| | KK |
| IMG_ | MNGIELKKEEAAFYFNQAELNLKAIEDNIFDKERRKTLLNNPQILAKMENFIFNFRDVTKNAKGEIDCLLLK |
| 3300031553 | LRELRNFYSHYVHKRDVRELSKGEKPILEKYYQFAIESTGSENVKLEIIENDAWLADAGVLFFLCIFLKKSQ |
| SEQ ID NO: | ANKLISGISGFKRNDDTGQPRRNLFTYFSIREGYKVVPEMQKHFLLFSLVNHLSNQDDYIEKAHQPYDIGEG |
| 4735 | LFFHRIASTFLNISGILRNMKFYTYQSKRLVEQRGELKREKDIFAWEEPFQGNSYFEINGHKGVIGEDELKEL |
| | CYAFLIGNQDANKVEGRITQFLEKFRNANSVQQVKDDEMLKPEYFPANYFAESGVGRIKDRVLNRLNKAI |
| | KSNKAKKGEIIAYDKMREVMAFINNSLPVDEKLKPKDYKRYLGMVRFWDREKDNIKREFETKEWSKYLPS |
| | NFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGWQE |
| | SEKVSKKIREAECEILLSKEYEELSKQFFQSKDYDKMTRINGLYEKNKLIALMAVYLMGQLRILFKEHTKLD |
| | DITKTTVDFKISDKVTVKIPFSNYPSLVYTMSSKYVDNIGNYGFSNKDKDKPILGKIDVIEKQRMEFIKEVLG |
| | FEKYLFDDKIIDKSKFADTATHISFAEIVEELVEKGWDKDRLTKLKDARNKALHGEILTGTSFDETKSLINEL |
| | KK |
| IMG_ | MNGIELKKEEAAFYFNQAELNLKAIEDNIFDKERRKTLLNNPQILAKMENFIFNFRDVTKNAKGEIDCLLLK |
| 3300031355_2 | LRELRNFYSHYVHKRDVRELSKGEKPILEKYYQFAIESTGSENVKLEIIENDAWLADAGVLFFLCIFLKKSQ |
| SEQ ID NO: | ANKLISGISGFKRNDDTGQPRRNLFTYFSIREGYKVVPEMQKHFLLFSLVNHLSNQDDYIEKAHQPYDIGEG |
| 4736 | LFFHRIASTFLNISGILRNMKFYTYQSKRLVEQRGELKREKDIFAWEEPFQGNSYFEINGHKGVIGEDELKEL |
| | CYAFLIGNQDANKVEGRITQFLEKFRNANSVQQVKDDEMLKPEYFPANYFAESGVGRIKDRVLNRLNKAI |
| | KSNKAKKGEIIAYDKMREVMAFINNSLPVDEKLKPKDYKRYLGMVRFWDREKDNIKREFETKEWSKYLPS |
| | NFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGWQE |
| | SEKVSKKIREAECEILLSKEYEELSKQFFQSKDYDKMTRINGLYEKNKLIALMAVYLMGQLRILFKEHTKLD |
| | DITKTTVDFKISDKVTVKIPFSNYPSLVYTMSSKYVDNIGNYGFSNKDKDKPILGKIDVIEKQRMEFIKEVLG |
| | FEKYLFDDKIIDKSKFADTATHISFAEIVEELVEKGWDKDRLTKLKDARNKALHGEILTGTSFDETKSLINEL |
| | KK |
| IMG_ | MNGIELKKEEAAFYFNQAELNLKAIEDNIFDKERRKTLLNNPQILAKMENFIFNFRDVTKNAKGEIDCLLLK |
| 33000315513 | LRELRNFYSHYVHKRDVRELSKGEKPILEKYYQFAIESTGSENVKLEIIENDAWLADAGVLFFLCIFLKKSQ |
| SEQ ID NO: | ANKLISGISGFKRNDDTGQPRRNLFTYFSIREGYKVVPEMQKHFLLFSLVNHLSNQDDYIEKAHQPYDIGEG |
| 4737 | LFFHRIASTFLNISGILRNMKFYTYQSKRLVEQRGELKREKDIFAWEEPFQGNSYFEINGHKGVIGEDELKEL |
| | CYAFLIGNQDANKVEGRITQFLEKFRNANSVQQVKDDEMLKPEYFPANYFAESGVGRIKDRVLNRLNKAI |
| | KSNKAKKGEIIAYDKMREVMAFINNSLPVDEKLKPKDYKRYLGMVRFWDREKDNIKREFETKEWSKYLPS |
| | NFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGWQE |
| | SEKVSKKIREAECEILLSKEYEELSKQFFQSKDYDKMTRINGLYEKNKLIALMAVYLMGQLRILFKEHTKLD |
| | DITKTTVDFKISDKVTVKIPFSNYPSLVYTMSSKYVDNIGNYGFSNKDKDKPILGKIDVIEKQRMEFIKEVLG |
| | FEKYLFDDKIIDKSKFADTATHISFAEIVEELVEKGWDKDRLTKLKDARNKALHGEILTGTSFDETKSLINEL |
| | KK |
| IMG_ | MNGIELKKEEAAFYFNQAELNLKAIEDNIFDKERRKTLLNNPQILAKMENFIFNFRDVTKNAKGEIDCLLLK |
| 3300031586_4 | LRELRNFYSHYVHKRDVRELSKGEKPILEKYYQFAIESTGSENVKLEIIENDAWLADAGVLFFLCIFLKKSQ |
| SEQ ID NO: | ANKLISGISGFKRNDDTGQPRRNLFTYFSIREGYKVVPEMQKHFLLFSLVNHLSNQDDYIEKAHQPYDIGEG |
| 4738 | LFFHRIASTFLNISGILRNMKFYTYQSKRLVEQRGELKREKDIFAWEEPFQGNSYFEINGHKGVIGEDELKEL |
| | CYAFLIGNQDANKVEGRITQFLEKFRNANSVQQVKDDEMLKPEYFPANYFAESGVGRIKDRVLNRLNKAI |
| | KSNKAKKGEIIAYDKMREVMAFINNSLPVDEKLKPKDYKRYLGMVRFWDREKDNIKREFETKEWSKYLPS |
| | NFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGWQE |
| | SEKVSKKIREAECEILLSKEYEELSKQFFQSKDYDKMTRINGLYEKNKLIALMAVYLMGQLRILFKEHTKLD |
| | DITKTTVDFKISDKVTVKIPFSNYPSLVYTMSSKYVDNIGNYGFSNKDKDKPILGKIDVIEKQRMEFIKEVLG |
| | FEKYLFDDKIIDKSKFADTATHISFAEIVEELVEKGWDKDRLTKLKDARNKALHGEILTGTSFDETKSLINEL |
| | KK |
| IMG_ | MNGIELKKEEAAFYFNQAELNLKAIEDNIFDKERRKTLLNNPQILAKMENFIFNFRDVTKNAKGEIDCLLLK |
| 3300031643_2 | LRELRNFYSHYVHKRDVRELSKGEKPILEKYYQFAIESTGSENVKLEIIENDAWLADAGVLFFLCIFLKKSQ |
| SEQ ID NO: | ANKLISGISGFKRNDDTGQPRRNLFTYFSIREGYKVVPEMQKHFLLFSLVNHLSNQDDYIEKAHQPYDIGEG |
| 4739 | LFFHRIASTFLNISGILRNMKFYTYQSKRLVEQRGELKREKDIFAWEEPFQGNSYFEINGHKGVIGEDELKEL |
| | CYAFLIGNQDANKVEGRITQFLEKFRNANSVQQVKDDEMLKPEYFPANYFAESGVGRIKDRVLNRLNKAI |
| | KSNKAKKGEIIAYDKMREVMAFINNSLPVDEKLKPKDYKRYLGMVRFWDREKDNIKREFETKEWSKYLPS |
| | NFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGWQE |
| | SEKVSKKIREAECEILLSKEYEELSKQFFQSKDYDKMTRINGLYEKNKLIALMAVYLMGQLRILFKEHTKLD |
| | DITKTTVDFKISDKVTVKIPFSNYPSLVYTMSSKYVDNIGNYGFSNKDKDKPILGKIDVIEKQRMEFIKEVLG |
| | FEKYLFDDKIIDKSKFADTATHISFAEIVEELVEKGWDKDRLTKLKDARNKALHGEILTGTSFDETKSLINEL |
| | KK |
| IMG_ | MNGIELKKEEAAFYFNQAELNLKAIEDNIFDKERRKTLLNNPQILAKMENFIFNFRDVTKNAKGEIDCLLLK |
| 3300031654_3 | LRELRNFYSHYVHKRDVRELSKGEKPILEKYYQFAIESTGSENVKLEIIENDAWLADAGVLFFLCIFLKKSQ |
| SEQ ID NO: | ANKLISGISGFKRNDDTGQPRRNLFTYFSIREGYKVVPEMQKHFLLFSLVNHLSNQDDYIEKAHQPYDIGEG |
| 4740 | LFFHRIASTFLNISGILRNMKFYTYQSKRLVEQRGELKREKDIFAWEEPFQGNSYFEINGHKGVIGEDELKEL |
| | CYAFLIGNQDANKVEGRITQFLEKFRNANSVQQVKDDEMLKPEYFPANYFAESGVGRIKDRVLNRLNKAI |
| | KSNKAKKGEIIAYDKMREVMAFINNSLPVDEKLKPKDYKRYLGMVRFWDREKDNIKREFETKEWSKYLPS |
| | NFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGWQE |
| | SEKVSKKIREAECEILLSKEYEELSKQFFQSKDYDKMTRINGLYEKNKLIALMAVYLMGQLRILFKEHTKLD |
| | DITKTTVDFKISDKVTVKIPFSNYPSLVYTMSSKYVDNIGNYGFSNKDKDKPILGKIDVIEKQRMEFIKEVLG |
| | FEKYLFDDKIIDKSKFADTATHISFAEIVEELVEKGWDKDRLTKLKDARNKALHGEILTGTSFDETKSLINEL |
| | KK |
| IMG_ | MNGIELKKEEAAFYFNQAELNLKAIEDNIFDKERRKTLLNNPQILAKMENFIFNFRDVTKNAKGEIDCLLLK |
| 33000316514 | LRELRNFYSHYVHKRDVRELSKGEKPILEKYYQFAIESTGSENVKLEIIENDAWLADAGVLFFLCIFLKKSQ |
| SEQ ID NO: | ANKLISGISGFKRNDDTGQPRRNLFTYFSIREGYKVVPEMQKHFLLFSLVNHLSNQDDYIEKAHQPYDIGEG |
| 4741 | LFFHRIASTFLNISGILRNMKFYTYQSKRLVEQRGELKREKDIFAWEEPFQGNSYFEINGHKGVIGEDELKEL |
| | CYAFLIGNQDANKVEGRITQFLEKFRNANSVQQVKDDEMLKPEYFPANYFAESGVGRIKDRVLNRLNKAI |
| | KSNKAKKGEIIAYDKMREVMAFINNSLPVDEKLKPKDYKRYLGMVRFWDREKDNIKREFETKEWSKYLPS |
| | NFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGWQE |
| | SEKVSKKIREAECEILLSKEYEELSKQFFQSKDYDKMTRINGLYEKNKLIALMAVYLMGQLRILFKEHTKLD |
| | DITKTTVDFKISDKVTVKIPFSNYPSLVYTMSSKYVDNIGNYGFSNKDKDKPILGKIDVIEKQRMEFIKEVLG |
| | FEKYLFDDKIIDKSKFADTATHISFAEIVEELVEKGWDKDRLTKLKDARNKALHGEILTGTSFDETKSLINEL |
| | KK |
| IMG_ | MNGIELKKEEAAFYFNQAELNLKAIEDNIFDKERRKTLLNNPQILAKMENFIFNFRDVTKNAKGEIDCLLLK |
| 3300031337 | LRELRNFYSHYVHKRDVRELSKGEKPILEKYYQFAIESTGSENVKLEIIENDAWLADAGVLFFLCIFLKKSQ |
| SEQ ID NO: | ANKLISGISGFKRNDDTGQPRRNLFTYFSIREGYKVVPEMQKHFLLFSLVNHLSNQDDYIEKAHQPYDIGEG |
| 4742 | LFFHRIASTFLNISGILRNMKFYTYQSKRLVEQRGELKREKDIFAWEEPFQGNSYFEINGHKGVIGEDELKEL |
| | CYAFLIGNQDANKVEGRITQFLEKFRNANSVQQVKDDEMLKPEYFPANYFAESGVGRIKDRVLNRLNKAI |
| | KSNKAKKGEIIAYDKMREVMAFINNSLPVDEKLKPKDYKRYLGMVRFWDREKDNIKREFETKEWSKYLPS |
| | NFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGWQE |
| | SEKVSKKIREAECEILLSKEYEELSKQFFQSKDYDKMTRINGLYEKNKLIALMAVYLMGQLRILFKEHTKLD |
| | DITKTTVDFKISDKVTVKIPFSNYPSLVYTMSSKYVDNIGNYGFSNKDKDKPILGKIDVIEKQRMEFIKEVLG |
| | FEKYLFDDKII |
| IMG_ | MNGIELKKEEAAFYFNQAELNLKAIEDNIFDKERRKTLLNNPQILAKMENFIFNFRDVTKNAKGEIDCLLLK |
| 3300031554_3 | LRELRNFYSHYVHKRDVRELSKGEKPILEKYYQFAIESTGSENVKLEIIENDAWLADAGVLFFLCIFLKKSQ |
| SEQ ID NO: | ANKLISGISGFKRNDDTGQPRRNLFTYFSIREGYKVVPEMQKHFLLFSLVNHLSNQDDYIEKAHQPYDIGEG |
| 4743 | LFFHRIASTFLNISGILRNMKFYTYQSKRLVEQRGELKREKDIFAWEEPFQGNSYFEINGHKGVIGEDELKEL |
| | CYAFLIGNQDANKVEGRITQFLEKFRNANSVQQVKDDEMLKPEYFPANYFAESGVGRIKDRVLNRLNKAI |
| | KSNKAKKGEIIAYDKMREVMAFINNSLPVDEKLKPKDYKRYLGMVRFWDREKDNIKREFETKEWSKYLPS |
| | NFWTAKNLERVYGLAREKNAELFNKLKADVEKMDERELEKYQKINDAKDLANLRRLASDFGVKWEEKD |
| | WDEYSGQIKKQITDSQKLTIMKQRITAGLKKKHGIENLNLRITIDINKSRKAVLNRIAIPRGFVKRHILGWQE |
| | SEKVSKKIREAECEILLSKEYEELSKQFFQSKDYDKMTRINGLYEKNKLIALMAVYLMGQLRILFKEHTKLD |
| | DITKTTVDFKISDKVTVKIPFSNYPSLVYTMSSKYVDNIGNYGFSNKDKDKPILGKIDVIEKQRMEFIKEVLG |
| | FEKYLFDDKIIDKSKFADTAT |
| IMG_ | MSGIELKKEEAAFYFNQAELNLKAIEVSIFDEGRRKTLLNNPKILAKVENFIFNSEDVTKNAKGEIDCLLSKL |
| 3300032020 | MELRNFYSHYVHKPDVKELSKGEKPILEKYYQFAIDATASADVKLEIIENDTWLTOAGVLLLLCMFLKKSQ |
| SEQ ID NO: | ANKLIGGISGFKRNDPTGQPRRNLFTYYSVREGYKVVPEMQKHFLLFALVNHLSNQDDYIEKAQQPYDIGE |
| 4744 | GLFFHRIASTFLDISGILRNMKFYTYQSKRLKEQRGELKREKDSFEWIEPFQGNSYFSVDGQKGVIGEDELKE |
| | LCYALLIGKQDANKVEGRITQFLKKFKNADDAQKVSDDEMLDRGNFPASYFAERRVGSIKDKILSSLEQAI |
| | KSYKTSGADVKAYNKMKEVMEFINNSLPVDEKLKRKDYKRYLGMVRLWGSERDNIKREFEAKGWSKYF |
| | TSGFWMAKNLERVYGLAREKNAELFNKLKTAVEKMDEREFVKYQQINDAKDLASLRQLANDFGVNWEE |
| | KDWEKYSGQIKKQITDSQKLTIMKQRITAGLKRKHGIENLNLRITIDSSKSRKAVLNRIAIPRGFVKKHILDW |
| | QGSEKVPKKIREAKCKILLSKEYEELSRQFYKVKDYDKMTQINSLYEKNKLIALMAVYLMEQLRIQLKEHT |
| | ELRNLDKTTVDFRISDKVTEKIPFSQYPSLVYAMSREYADNVDNYKFSEEDKKKLDKIKKNLFLGKIDIIEK |
| | QRMEFIKEVLGFEEYLFDDKIIDRSKFADTATHISFGEIVGELIGKGWDKDKLTKLEYARNKALHGEIPEATS |
| | FNEAKQLINELKK |
| IMG_ | MENIKLEKQKAAFYFNQAELNLKAIEGNIFDKGRRKTLFDNPKILSKVENFIFNFKDVTKNAKGEIDCLLSK |
| 3300032029 | LMELRNFYSHYVHKPDVKELSKGEKPLLERYYQIAIEATGSENVKLEIIENDKWLTDAGVLLFLCMFLKKS |
| SEQ ID NO: | QANKLISGISGFKRNDTFGQPRRNLFNYFSVRERYKVVPDMQKHFLLFVLVNHLSEQDDYIEKAQQPYNIG |
| 4745 | EGLFFHRIASTFLNVSGILRNMEFYTYQSKRLKEQRGELKREKDIFTWEEPFQGNSYFEINGHKGVIGEDELK |
| | ELCYALLSYNKSKYAVEQIEKFLKGFGEVKSEQEIRDSDILNESYFPTNYFAESNIGSIKEKILNRLGKTODSY |
| | KKTGTKIKPYDMMKEVMEFINNSLPADEKLKRKDYRRYLKMVRIWDSEKDNIKREFESKEWSKYFSSNFW |
| | MAKNLERVYGLAREKNAELFNKLKAVVEKMDEREFEKYRQINSAEDLASLRRLANDYGVKWEEKDWQE |
| | YSGQIKKQISDRQKLTIMKQRITAELKKKHGIENLNLRITIDSNKSRKAVLNRIAVPRGFVKEHILGWQGSEK |
| | VSKKTREAKCKILLSKEYEELSKQFFQTRNYDKMTQVNSLYEKNKLIAFMAVYRAIEHPV |
| IMG_ | MENIKLEKQKAAFYFNQAELNLKAIEGNIFDKGRRKTLFDNPKILSKVENFIFNFKDVTKNAKGEIDCLLSK |
| 3300031586_2 | LMELRNFYSHYVHKPDVKELSKGEKPLLERYYQIAIEATGSENVKLEIIENDKWLTDAGVLLFLCMFLKKS |
| SEQ ID NO: | QANKLISGISGFKRNDTFGQPRRNLFNYFSVRERYKVVPDMQKHFLLFVLVNHLSEQDDYIEKAQQPYNIG |
| 4746 | EGLFFHRIASTFLNVSGILRNMEFYTYQSKRLKEQRGELKREKDIFTWEEPFQGNSYFEINGHKGVIGEDELK |
| | ELCYALLSYNKSKYAVEQIEKFLKGFGEVKSEQEIRDSDILNESYFPTNYFAESNIGSIKEKILNRLGKTODSY |
| | KKTGTKIKPYDMMKEVMEFINNSLPADEKLKRKDYRRYLKMVRIWDSEKDNIKREFESKEWSKYFSSNFW |
| | MAKNLERVYGLAREKNAELFNKLKAVVEKMDEREFEKYRQINSAEDLASLRRLANDYGVKWEEKDWQE |
| | YSGQIKKQISDRQKLTIMKQRITAELKKKHGIENLNLRITIDSNKSRKAVLNRIAVPRGFVKEHILGWQGSEK |
| | VSKKTREAKCKILLSKEYEELSKQFFQTRNYDKMTQVNSLYEKNKLIAFMAVYLMGQLNIRFDKPTRLNEL |
| | EKAEVDFKISDKVTAKIPFSQYPSLVYAMSSKYADSVGSYKFENDEKNKPFLGKIDIIEKQRMEFIKEVLGFE |
| | EYLFEKKIIDKSKFADTATHISFREICDELIQKGWDENKLTNLKDARNAALHGEIPAETSFREAKPLINGLKK |
| IMG_ | MENIKLEKQKAAFYFNQAELNLKAIEGNIFDKGRRKTLFDNPKILSKVENFIFNFKDVTKNAKGEIDCLLSK |
| 33000315512 | LMELRNFYSHYVHKPDVKELSKGEKPLLERYYQIAIEATGSENVKLEIIENDKWLTDAGVLLFLCMFLKKS |
| SEQ ID NO: | QANKLISGISGFKRNDTFGQPRRNLFNYFSVRERYKVVPDMQKHFLLFVLVNHLSEQDDYIEKAQQPYNIG |
| 4747 | EGLFFHRIASTFLNVSGILRNMEFYTYQSKRLKEQRGELKREKDIFTWEEPFQGNSYFEINGHKGVIGEDELK |
| | ELCYALLSYNKSKYAVEQIEKFLKGFGEVKSEQEIRDSDILNESYFPTNYFAESNIGSIKEKILNRLGKTODSY |
| | KKTGTKIKPYDMMKEVMEFINNSLPADEKLKRKDYRRYLKMVRIWDSEKDNIKREFESKEWSKYFSSNFW |
| | MAKNLERVYGLAREKNAELFNKLKAVVEKMDEREFEKYRQINSAEDLASLRRLANDYGVKWEEKDWQE |
| | YSGQIKKQISDRQKLTIMKQRITAELKKKHGIENLNLRITIDSNKSRKAVLNRIAVPRGFVKEHILGWQGSEK |
| | VSKKTREAKCKILLSKEYEELSKQFFQTRNYDKMTQVNSLYEKNKLIAFMAVYLMGQLNIRFDKPTRLNEL |
| | EKAEVDFKISDKVTAKIPFSQYPSLVYAMSSKYADSVGSYKFENDEKNKPFLGKIDIIEKQRMEFIKEVLGFE |
| | EYLFEKKIIDKSKFADTATHISFREICDELIQKGWDENKLTNLKDARNAALHGEIPAETSFREAKPLINGLKK |
| IMG_ | MENIKLEKQKAAFYFNQAELNLKAIEGNIFDKGRRKTLFDNPKILSKVENFIFNFKDVTKNAKGEIDCLLSK |
| 3300031624_4 | LMELRNFYSHYVHKPDVKELSKGEKPLLERYYQIAIEATGSENVKLEIIENDKWLTDAGVLLFLCMFLKKS |
| SEQ ID NO: | QANKLISGISGFKRNDTFGQPRRNLFNYFSVRERYKVVPDMQKHFLLFVLVNHLSEQDDYIEKAQQPYNIG |
| 4748 | EGLFFHRIASTFLNVSGILRNMEFYTYQSKRLKEQRGELKREKDIFTWEEPFQGNSYFEINGHKGVIGEDELK |
| | ELCYALLSYNKSKYAVEQIEKFLKGFGEVKSEQEIRDSDILNESYFPTNYFAESNIGSIKEKILNRLGKTODSY |
| | KKTGTKIKPYDMMKEVMEFINNSLPADEKLKRKDYRRYLKMVRIWDSEKDNIKREFESKEWSKYFSSNFW |
| | MAKNLERVYGLAREKNAELFNKLKAVVEKMDEREFEKYRQINSAEDLASLRRLANDYGVKWEEKDWQE |
| | YSGQIKKQISDRQKLTIMKQRITAELKKKHGIENLNLRITIDSNKSRKAVLNRIAVPRGFVKEHILGWQGSEK |
| | VSKKTREAKCKILLSKEYEELSKQFFQTRNYDKMTQVNSLYEKNKLIAFMAVYLMGQLNIRFDKPTRLNEL |
| | EKAEVDFKISDKVTAKIPFSQYPSLVYAMSSKYADSVGSYKFENDEKNKPFLGKIDIIEKQRMEFIKEVLGFE |
| | EYLFEKKIIDKSKFADTATHISFREICDELIQKGWDENKLTNLKDARNAALHGEIPAETSFREAKPLINGLKK |
| IMG_ | MENIKLEKQKAAFYFNQAELNLKAIEGNIFDKGRRKTLFDNPKILSKVENFIFNFKDVTKNAKGEIDCLLSK |
| 3300031650_3 | LMELRNFYSHYVHKPDVKELSKGEKPLLERYYQIAIEATGSENVKLEIIENDKWLTDAGVLLFLCMFLKKS |
| SEQ ID NO: | QANKLISGISGFKRNDTFGQPRRNLFNYFSVRERYKVVPDMQKHFLLFVLVNHLSEQDDYIEKAQQPYNIG |
| 4749 | EGLFFHRIASTFLNVSGILRNMEFYTYQSKRLKEQRGELKREKDIFTWEEPFQGNSYFEINGHKGVIGEDELK |
| | ELCYALLSYNKSKYAVEQIEKFLKGFGEVKSEQEIRDSDILNESYFPTNYFAESNIGSIKEKILNRLGKTODSY |
| | KKTGTKIKPYDMMKEVMEFINNSLPADEKLKRKDYRRYLKMVRIWDSEKDNIKREFESKEWSKYFSSNFW |
| | MAKNLERVYGLAREKNAELFNKLKAVVEKMDEREFEKYRQINSAEDLASLRRLANDYGVKWEEKDWQE |
| | YSGQIKKQISDRQKLTIMKQRITAELKKKHGIENLNLRITIDSNKSRKAVLNRIAVPRGFVKEHILGWQGSEK |
| | VSKKTREAKCKILLSKEYEELSKQFFQTRNYDKMTQVNSLYEKNKLIAFMAVYLMGQLNIRFDKPTRLNEL |
| | EKAEVDFKISDKVTAKIPFSQYPSLVYAMSSKYADSVGSYKFENDEKNKPFLGKIDIIEKQRMEFIKEVLGFE |
| | EYLFEKKIIDKSKFADTATHISFREICDELIQKGWDENKLTNLKDARNAALHGEIPAETSFREAKPLINGLKK |
| IMG_ | MENIKLEKQKAAFYFNQAELNLKAIEGNIFDKGRRKTLFDNPKILSKVENFIFNFKDVTKNAKGEIDCLLSK |
| 3300031554_2 | LMELRNFYSHYVHKPDVKELSKGEKPLLERYYQIAIEATGSENVKLEIIENDKWLTOAGVLLFLCMFLKKS |
| SEQ ID NO: | QANKLISGISGFKRNDTFGQPRRNLFNYFSVRERYKVVPDMQKHFLLFVLVNHLSEQDDYIEKAQQPYNIG |
| 4750 | EGLFFHRIASTFLNVSGILRNMEFYTYQSKRLKEQRGELKREKDIFTWEEPFQGNSYFEINGHKGVIGEDELK |
| | ELCYALLSYNKSKYAVEQIEKFLKGFGEVKSEQEIRDSDILNESYFPTNYFAESNIGSIKEKILNRLGKTODSY |
| | KKTGTKIKPYDMMKEVMEFINNSLPADEKLKRKDYRRYLKMVRIWDSEKDNIKREFESKEWSKYFSSNFW |
| | MAKNLERVYGLAREKNAELFNKLKAVVEKMDEREFEKYRQINSAEDLASLRRLANDYGVKWEEKDWQE |
| | YSGQIKKQISDRQKLTIMKQRITAELKKKHGIENLNLRITIDSNKSRKAVLNRIAVPRGFVKEHILGWQGSEK |
| | VSKKTREAKCKILLSKEYEELSKQFFQTRNYDKMTQVNSLYEKNKLIAFMAVYLMGQLNIRFDKPTRLNEL |
| | EKAEVDFKISDKVTAKIPFSQYPSLVYAMSSKYADSVGSYKFENDEKNKPFLGKIDIIEKQRMEFIKEVLGFE |
| | EYLFEKKIIDKSKFADTATHISFREICDELIQKGWDENKLTNLKDARNAALHGEIPAETSFREAKPLINGLKK |
| IMG_ | MSPDFIKLEKQEAAFYFNQTELNLKAIESNIFDKQQRVILLNNPQILAKVGDFIFNFRDVTKNAKGEIDCLLL |
| 3300031331 | KLRELRNFYSHYVYTDDVKILSNGERPLLEKYYQFAIEATGSENVKLEIIESNNRLTEAGVLFFLCMFLKKS |
| SEQ ID NO: | QANKLISGISGFKRNDPTGQPRRNLFTYFSVREGYKVVPDMQKHFLLFVLVNHLSGQDDYIEKAQKPYDIG |
| 4751 | EGLFFHRIASTFLNISGILRNMEFYIYQSKRLKEQQGELKREKDIFPWIEPFQGNSYFEINGNKGIIGEDELKEL |
| | CYALLVAGKDVRAVEGKITQFLEKFKNADNAQQVEKDEMLDRNNFPANYFAESNIGSIKEKILNRLGKTD |
| | DSYNKTGTKIKPYDMMKEVMEFINNSLPADEKLKRKDYRRYLKMVRIWDSEKDNIKREFESKEWSKYFSS |
| | DFWMAKNLERVYGLAREKNAELFNKLKAVVEKMDEREFEKYRLINSAEDLASLRRLAKDFGLKWEEKD |
| | WQEYSGQIKKQISDRQKLTIMKQRITAELKKKHGIENLNLRITIDSNKSRKAVLNRIAVPRGFVKEHILGWQ |
| | GSEKVSKKTREAKCKILLSKEYEELSKQFFQTRNYDKMTQVNGLYEKNKLLAFMVVYLMERLNILLNKPT |
| | ELNELEKAEVDFKISDKVMAKIPFSQYPSLVYAMSSKYADSVGSYKFENDEKNKPFLGKIDTIEKQRMEFIK |
| | EVLGFEEYLFEKKIIDKSEFADTATHISFDE |
| IMG_ | MSPDFIKLEKQEAAFYFNQTELNLKAIESNIFDKQQRVILLNNPQILAKVGDFIFNFRDVTKNAKGEIDCLLL |
| 3300031278_2 | KLRELRNFYSHYVYTDDVKILSNGERPLLEKYYQFAIEATGSENVKLEIIESNNRLTEAGVLFFLCMFLKKS |
| SEQ ID NO: | QANKLISGISGFKRNDPTGQPRRNLFTYFSVREGYKVVPDMQKHFLLFVLVNHLSGQDDYIEKAQKPYDIG |
| 4752 | EGLFFHRIASTFLNISGILRNMEFYIYQSKRLKEQQGELKREKDIFPWIEPFQGNSYFEINGNKGIIGEDELKEL |
| | CYALLVAGKDVRAVEGKITQFLEKFKNADNAQQVEKDEMLDRNNFPANYFAESNIGSIKEKILNRLGKTD |
| | DSYNKTGTKIKPYDMMKEVMEFINNSLPADEKLKRKDYRRYLKMVRIWDSEKDNIKREFESKEWSKYFSS |
| | DFWMAKNLERVYGLAREKNAELFNKLKAVVEKMDEREFEKYRLINSAEDLASLRRLAKDFGLKWEEKD |
| | WQEYSGQIKKQISDRQKLTIMKQRITAELKKKHGIENLNLRITIDSNKSRKAVLNRIAVPRGFVKEHILGWQ |
| | GSEKVSKKTREAKCKILLSKEYEELSKQFFQTRNYDKMTQVNGLYEKNKLLAFMVVYLMERLNILLNKPT |
| | ELNELEKAEVDFKISDKVMAKIPFSQYPSLVYAMSSKYADSVGSYKFENDEKNKPFLGKIDTIEKQRMEFIK |
| | EVLGFEEYLFEKKIIDKSEFADTATHISFDEICNELIKKGWDKDKLTKLKDARNAALHGEIPAETSFREAKPLI |
| | NGLKK |
| IMG_ | MSPDFIKLEKQEAAFYFNQTELNLKAIESNILDKQQRMILLNNPRILAKVGNFIFNFRDVTKNAKGEIDCLLF |
| 3300031575_3 | KLEELRNFYSHYVHTDNVKELSNGEKPLLERYYQIAIQATRSEDVKFELFETRNENKITDAGVLFFLCMFLK |
| SEQ ID NO: | KSQANKLISGISGFKRNDPTGQPRRNLFTYFSAREGYKALPDMQKHFLLFTLVNYLSNQDEYISELKQYGEI |
| 4753 | GQGAFFNRIASTFLNISGISGNTKFYSYQSKRIKEQRGELNSEKDSFEWIEPFQGNSYFEINGHKGVIGEDELK |
| | ELCYALLVAKQDINAVEGKIMQFLKKFRNTGNLQQVKDDEMLEIEYFPASYFNESKKEDIKKEILGRLDKKI |
| | RSCSAKAEKAYDKMKEVMEFINNSLPAEEKLKRKDYRRYLKMVRFWSREKGNIEREFRTKEWSKYFSSDF |
| | WRKNNLEDVYKLATQKNAELFKNLKAAAEKMGETEFEKYQQINDVKDLASLRRLTQDFGLKWEEKDWE |
| | EYSEQIKKQITDRQKLTIMKQRVTAELKKKHGIENLNLRITIDSNKSRKAVLNRIAIPRGFVKKHILGWQGSE |
| | KISKNIREAECKILLSKKYEELSRQFFEAGNFDKLTQINGLYEKNKLTAFMSVYLMGRLNIQLNKHTELGNL |
| | KKTEVDFKISDKVTEKIPFSQYPSLVYAMSRKYVDNVDKYKFSHQDKKKPFLGKIDSIEKERIEFIKEVLDFE |
| | EYLFKNKVIDKSKFSDTATHISFKEICDEMGKKGCNRNKLTELNNARNAALHGEIPSETSFREAKPLINELK |
| | K |
| IMG_ | MSPDFIKLEKQEAAFYFNQTELNLKAIESNILDKQQRMILLNNPRILAKVGNFIFNFRDVTKNAKGEIDCLLF |
| 3300031356_2 | KLEELRNFYSHYVHTDNVKELSNGEKPLLERYYQIAIQATRSEDVKFELFETRNENKITDAGVLFFLCMFLK |
| SEQ ID NO: | KSQANKLISGISGFKRNDPTGQPRRNLFTYFSAREGYKALPDMQKHFLLFTLVNYLSNQDEYISELKQYGEI |
| 4754 | GQGAFFNRIASTFLNISGISGNTKFYSYQSKRIKEQRGELNSEKDSFEWIEPFQGNSYFEINGHKGVIGEDELK |
| | ELCYALLVAKQDINAVEGKIMQFLKKFRNTGNLQQVKDDEMLEIEYFPASYFNESKKEDIKKEILGRLDKKI |
| | RSCSAKAEKAYDKMKEVMEFINNSLPAEEKLKRKDYRRYLKMVRFWSREKGNIEREFRTKEWSKYFSSDF |
| | WRKNNLEDVYKLATQKNAELFKNLKAAAEKMGETEFEKYQQINDVKDLASLRRLTQDFGLKWEEKDWE |
| | EYSEQIKKQITDRQKLTIMKQRVTAELKKKHGIENLNLRITIDSNKSRKAVLNRIAIPRGFVKKHILGWQGSE |
| | KISKNIREAECKILLSKKYEELSRQFFEAGNFDKLTQINGLYEKNKLTAFMSVYLMGRLNIQLNKHTELGNL |
| | KKTEVDFKISDKVTEKIPFSQYPSLVYAMSRKYVDNVDKYKFSHQDKKKPFLGKIDSIEKERIEFIKEVLDFE |
| | EYLFKNKVIDKSKFSDTATHISFKEICDEMGKKGCNRNKLTELNNARNAALHGEIPSETSFREAKPLINELK |
| | K |
| IMG_ | MSPDFIKLEKQEAAFYFNQTELNLKAIESNILDKQQRMILLNNPRILAKVGNFIFNFRDVTKNAKGEIDCLLF |
| 3300031358_2 | KLEELRNFYSHYVHTDNVKELSNGEKPLLERYYQIAIQATRSEDVKFELFETRNENKITDAGVLFFLCMFLK |
| SEQ ID NO: | KSQANKLISGISGFKRNDPTGQPRRNLFTYFSAREGYKALPDMQKHFLLFTLVNYLSNQDEYISELKQYGEI |
| 4755 | GQGAFFNRIASTFLNISGISGNTKFYSYQSKRIKEQRGELNSEKDSFEWIEPFQGNSYFEINGHKGVIGEDELK |
| | ELCYALLVAKQDINAVEGKIMQFLKKFRNTGNLQQVKDDEMLEIEYFPASYFNESKKEDIKKEILGRLDKKI |
| | RSCSAKAEKAYDKMKEVMEFINNSLPAEEKLKRKDYRRYLKMVRFWSREKGNIEREFRTKEWSKYFSSDF |
| | WRKNNLEDVYKLATQKNAELFKNLKAAAEKMGETEFEKYQQINDVKDLASLRRLTQDFGLKWEEKDWE |
| | EYSEQIKKQITDRQKLTIMKQRVTAELKKKHGIENLNLRITIDSNKSRKAVLNRIAIPRGFVKKHILGWQGSE |
| | KISKNIREAECKILLSKKYEELSRQFFEAGNFDKLTQINGLYEKNKLTAFMSVYLMGRLNIQLNKHTELGNL |
| | KKTEVDFKISDKVTEKIPFSQYPSLVYAMSRKYVDNVDKYKFSHQDKKKPFLGKIDSIEKERIEFIKEVLDFE |
| | EYLFKNKVIDKSKFSDTATHISFKEICDEMGKKGCNRNKLTELNNARNAALHGEIPSETSFREAKPLINELK |
| | K |
| IMG_ | MINIELKKEEAAFYFNQANLNISGLDEVIEKQLPHIGSKKENAKKAIDKIFDNITVLKKVENFVFYFKDVAK |
| 3300031620_3 | NERVELDALLLKLIDLRNFYSHYVHNDNVKILSDGEETLLEKYYQIAIEATGSKDVKLEIIDNEKKLTDAGIL |
| SEQ ID NO: | FLLCMFLKKSQANKLISSISGFKRNDKEWQPRRNLFTYYSLREGYKVVPDMEKHFLLFTLVNHLSTQDENIE |
| 4756 | NTQPSDDIGRGLFFHRIASTFLNISGIFNNMEFYPYQSNRLKERRGDIAPDKDSFAWIEPFQGNSYFKINGYK |
| | GVIGENELKELCFAVLLHNKSKYAVEQIEKFLKCFKEVQSKQEIIECDILDECYFPANYLNQPETKSLKEKLL |
| | SRITGKINYSFDTAEKAFDKMKDVMEFINGCLPSDEKLKRKDYSRYLKMVRFWGGEKDNIKREFEGKKWT |
| | RFFPSELWHKRTLEDVYKFALKKNKKRLEELKVKIEGLNEDDLLKYQKVNNIKNLENLRLLAHDLDLSWR |
| | EKDWGEYSGQIKKQISDNQKLTIMKQRVIAELKKKHGIENINLRISLDSNKSIQAVLNRIAIPKGFIKRHVLH |
| | LQENEKTSRKIREAKCKILLSKKYEYLSRKFLDEKNFDKLTQINGLYEKNRLIAFMVIYLLKKLGLELKNET |
| | KLIELKKTRVKYKISDKVAEDIPLSHYPSLVYAMSRKYVDNIDNYEFPDEYAKKAILDKVDIIENQRMEFIK |
| | QVLGFEKYLFDNNIIDKSKFTDVETHISFVKIHDELIEKGWDTEKLSKLKHARNKALHGEIPGGTSFEKAKLL |
| | INELKK |
| IMG_ | MINIELKKEEAAFYFNQANLNISGLDEVIEKQLPHIGSKKENAKKAIDKIFDNITVLKKVENFVFYFKDVAK |
| 3300031586 | NERVELDALLLKLIDLRNFYSHYVHNDNVKILSDGEETLLEKYYQIAIEATGSKDVKLEIIDNEKKLTDAGIL |
| SEQ ID NO: | FLLCMFLKKSQANKLISSISGFKRNDKEWQPRRNLFTYYSLREGYKVVPDMEKHFLLFTLVNHLSTQDENIE |
| 4757 | NTQPSDDIGRGLFFHRIASTFLNISGIFNNMEFYPYQSNRLKERRGDIAPDKDSFAWIEPFQGNSYFKINGYK |
| | GVIGENELKELCFAVLLHNKSKYAVEQIEKFLKCFKEVQSKQEIIECDILDECYFPANYLNQPETKSLKEKLL |
| | SRITGKINYSFDTAEKAFDKMKDVMEFINGCLPSDEKLKRKDYSRYLKMVRFWGGEKDNIKREFEGKKWT |
| IMG_ | MINIELKKEEAAFYFNQANLNISGLDEVIEKQLPHIGSKKENAKKAIDKIFDNITVLKKVENFVFYFKDVAK |
| 3300031650 | NERVELDALLLKLIDLRNFYSHYVHNDNVKILSDGEETLLEKYYQIAIEATGSKDVKLEIIDNEKKLTDAGIL |
| SEQ ID NO: | FLLCMFLKKSQANKLISSISGFKRNDKEWQPRRNLFTYYSLREGYKVVPDMEKHFLLFTLVNHLSTQDENIE |
| 4758 | NTQPSDDIGRGLFFHRIASTFLNISGIFNNMEFYPYQSNRLKERRGDIAPDKDSFAWIEPFQGNSYFKINGYK |
| | GVIGENELKELCFAVLLHNKSKYAVEQIEKFLKCFKEVQSKQEIIECDILDECYFPANYLNQPETKSLKEKLL |
| | SRITGKINYSFDTAEKAFDKMKDVMEFINGCLPSDEKLKRKDYSRYLKMVRFWGGEKDNIKREFEGKKWT |
| | RFFPSELWHKR |
| IMG_ | MINIELKKEEAAFYFNQANLNISGLDEVIEKQLPHIGSKKENAKKAIDKIFDNITVLKKVENFVFYFKDVAK |
| 3300031624 | NERVELDALLLKLIDLRNFYSHYVHNDNVKILSDGEETLLEKYYQIAIEATGSKDVKLEIIDNEKKLTDAGIL |
| SEQ ID NO: | FLLCMFLKKSQANKLISSISGFKRNDKEWQPRRNLFTYYSLREGYKVVPDMEKHFLLFTLVNHLSTQDENIE |
| 4759 | NTQPSDDIGRGLFFHRIASTFLNISGIFNNMEFYPYQSNRLKERRGDIAPDKDSFAWIEPFQGNSYFKINGYK |
| | GVIGENELKELCFAVLLHNKSKYAVEQIEKFLKCFKEVQSKQEIIECDILDECYFPANYLNQPETKSLKEKLL |
| | SRITGKINYSFDTAEKAFDKMKDVMEFINGCLPSDEKLKRKDYSRYLKMVRFWGGEKDNIKREFEGKKWT |
| | RFFPSELWHKRTLEDVYKFALKKNKKRLEELKVKIEGLNEDDLLKYQKVNNIKNLENLRLLAHDLDLSWR |
| | EKDWGEYSGQIKKQISDNQKLTIMKQRVIAELKKKHGIENINLRISLDSNKSIQAVLNRIAIPKGFIKRHVLH |
| | LQENEKTSRKIREAKCKILLSKKYEYLSRKFLDEKNFDKLTQINGLYEKNRLIAFMVIYLLKQLGLELKNET |
| | KLIELKKTRVKYKISDKVAEDIPLSHYPSLVYAMSRKYVDNIDNYEFPDEYAKKAILDKVDIIENQRMEFIK |
| | QVLGFEKYLFDNNIIDKSKFTDVETHISFVKIHDELIEKGWDTEKLSKLKHARNKALHGEIPGGTSFEKAKLL |
| | INELKK |
| IMG_ | MINIELKKEEAAFYFNQANLNISGLDEVIEKQLPHIGSKKENAKKAIDKIFDNITVLKKVENFVFYFKDVAK |
| 3300031551 | NERVELDALLLKLIDLRNFYSHYVHNDNVKILSDGEETLLEKYYQIAIEATGSKDVKLEIIDNEKKLTDAGIL |
| SEQ ID NO: | FLLCMFLKKSQANKLISSISGFKRNDKEWQPRRNLFTYYSLREGYKVVPDMEKHFLLFTLVNHLSTQDENIE |
| 4760 | NTQPSDDIGRGLFFHRIASTFLNISGIFNNMEFYPYQSNRLKERRGDIAPDKDSFAWIEPFQGNSYFKINGYK |
| | GVIGENELKELCFAVLLHNKSKYAVEQIEKFLKCFKEVQSKQEIIECDILDECYFPANYLNQPETKSLKEKLL |
| | SRITGKINYSFDTAEKAFDKMKDVMEFINGCLPSDEKLKRKDYSRYLKMVRFWGGEKDNIKREFEGKKWT |
| | RFFPSELWHKRTLEDVYKFALKKNKKRLEELKVKIEGLNEDDLLKYQKVNNIKNLENLRLLAHDLDLSWR |
| | EKDWGEYSGQIKKKQISDNQKLTIMKQRVIAELKKKHGIENINLRISLDSNKSIQAVLNRIAIPKGFIKRHVLH |
| | LQENEKTSRKIREAKCKILLSKKYEYLSRKFLDEKNFDKLTQINGLYEKNRLIAFMVIYLLKQLGLELKNET |
| | KLIELKKTRVKYKISDKVAEDIPLSHYPSLVYAMSRKYVDNIDNYEFPDEYAKKAILDKVDIIENQRMEFIK |
| | QVLGFEKYLFDNNIIDKSKFTOVETHISFVKIHDELIEKGWDTEKLSKLKHARNKALHGEIPGGTSFEKAKLL |
| | INELKK |
| IMG_ | MINIELKKEEAAFYFNQANLNISGLDEVIEKQLPHIGSKKENAKKAIDKIFDNITVLKKVENFVFYFKDVAK |
| 3300032062_2 | NERVELDALLLKLIDLRNFYSHYVHNDNVKILSDGEETLLEKYYQIAIEATGSKDVKLEIIDNEKKLTDAGIL |
| SEQ ID NO: | FLLCMFLKKSQANKLISSISGFKRNDKEWQPRRNLFTYYSLREGYKVVPDMEKHFLLFTLVNHLSTQDENIE |
| 4761 | NTQPSDDIGRGLFFHRIASTFLNISGIFNNMEFYPYQSNRLKERRGDIAPDKDSFAWIEPFQGNSYFKINGYK |
| | GVIGENELKELCFAVLLHNKSKYAVEQIEKFLKCFKEVQSKQEIIECDILDECYFPANYLNQPETKSLKEKLL |
| | SRITGKINYSFDTAEKAFDKMKDVMEFINGCLPSDEKLKRKDYSRYLKMVRFWGGEKDNIKREFEGKKWT |
| | RFFPSELWHKRTLEDVYKFALKKNKKRLEELKVKIEGLNEDDLLKYQKVNNIKNLENLRLLAHDLDLSWR |
| | EKDWGEYSGQIKKQISDNQKLTIMKQRVIAELKKKHGIENINLRISLDSNKSIQAVLNRIAIPKGFIKRHVLH |
| | LQENEKTSRKIREAKCKILLSKKYEYLSRKFLDEKNFDKLTQINGLYEKNRLIAFMVIYLLKQLGLELKNET |
| | KLIELKKTRVKYKISDKVAEDIPLSHYPSLVYAMSRKYVDNIDNYEFPDEYAKKAILDKVDIIENQRMEFIK |
| | QVLGFEKYLFDNNIIDKSKFTDVETHISFVKIHDELIEKGWDTEKLSKLKHARNKALHGEIPGGTSFEKAKLL |
| | INELKK |
| IMG_ | MINIELKKEEAAFYFNQANLNISGLDEVIEKQLPHIGSKKENAKKAIDKIFDNITVLKKVENFVFYFKDVAK |
| 3300031554 | NERVELDALLLKLIDLRNFYSHYVHNDNVKILSDGEETLLEKYYQIAIEATGSKDVKLEIIDNEKKLTDAGIL |
| SEQ ID NO: | FLLCMFLKKSQANKLISSISGFKRNDKEWQPRRNLFTYYSLREGYKVVPDMEKHFLLFTLVNHLSTQDENIE |
| 4762 | NTQPSDDIGRGLFFHRIASTFLNISGIFNNMEFYPYQSNRLKERRGDIAPDKDSFAWIEPFQGNSYFKINGYK |
| | GVIGENELKELCFAVLLHNKSKYAVEQIEKFLKCFKEVQSKQEIIECDILDECYFPANYLNQPETKSLKEKLL |
| | SRITGKINYSFDTAEKAFDKMKDVMEFINGCLPSDEKLKRKDYSRYLKMVRFWGGEKDNIKREFEGKKWT |
| | RFFPSELWHKRTLEDVYKFALKKNKKRLEELKVKIEGLNEDDLLKYQKVNNIKNLENLRLLAHDLDLSWR |
| | EKDWGEYSGQIKKQISDNQKLTIMKQRVIAELKKKHGIENINLRISLDSNKSIQAVLNRIAIPKGFIKRHVLH |
| | LQENEKTSRKIREAKCKILLSKKYEYLSRKFLDEKNFDKLTQINGLYEKNRLIAFMVIYLLKQLGLELKNET |
| | KLIELKKTRVKYKISDKVAEDIPLSHYPSLVYAMSRKYVDNIDNYEFPDEYAKKAILDKVDIIENQRMEFIK |
| | QVLGFEKYLFDNNIIDKSKFTDVETHISFVKIHDELIEKGWDTEKLSKLKHARNKALHGEIPGGTSFEKAKLL |
| | INELKK |
| IMG_ | MNIIKLKKEEAAFYFNQTILNLSGLDEIIEKQIPHIISNKENAKKVIDKIFNNRLLLKSVENYIYNFKDVAKNA |
| 3300017991 | RTEIEAILLKLVELRNFYSHYVHNDTVKILSNGEKPILEKYYQIAIEATGSKNVKLVIIENNNCLTDSGVLFLL |
| SEQ ID NO: | CMFLKKSQANKLISSVSGFKRNDKEGQPRRNLFTYYSVREGYKVVPDMQKHFLLFALVNHLSEQDDHIEK |
| 4763 | QQQSDELGKGLFFHRIASTFLNESGIFNKMQFYTYQSNRLKEKRGELKHEKDTFTWIEPFQGNSYFTLNGH |
| | KGVISEDQLKELCYTILIEKQNVDSLEGKIIQFLKKFQNVSSKQQVDEDELLKREYFPANYFGRAGTGTLKE |
| | KILNRLDKRMDPTSKVTDKAYDKMIEVMEFINMCLPSDEKLRQKDYRRYLKMVRFWNKEKHNIKREFDS |
| | KKWTRFLPTELWNKRNLEEAYQLARKENKKKLEDMRNQVRSLKENDLEKYQQINYVNDLENLRLLSQEL |
| | GVKWQEKDWVEYSGQIKKQISDNQKLTIMKQRITAELKKMHGIENLNLRISIDTNKSRQTVMNRIALPKGF |
| | VKNHIQQNSSEKISKRIREDYCKIELSGKYEELSRQFFDKKNFDKMTLINGLCEKNKLIAFMVIYLLERLGFE |
| | LKEKTKLGELKQTRMTYKISDKVKEDIPLSYYPKLVYAMNRKYVDNIDSYAFAAYESKKAILDKVDIIEKQ |
| | RMEFIKQVLCFEEYIFENRIIEKSKFNDEETHISFTQIHDELIKKGRDTEKLSKLKHARNKALHGEIPDGTSFE |
| | KAKLLINEIKK |
| IMG_ | MNIIKLKKEEAAFYFNQTILNLSGLDEIIEKQIPHIISNKENAKKVIDKIFNNRLLLKSVENYIYNFKDVAKNA |
| 3300018080 | RTEIEAILLKLVELRNFYSHYVHNDTVKILSNGEKPILEKYYQIAIEATGSKNVKLVIIENNNCLTDSGVLFLL |
| SEQ ID NO: | CMFLKKSQANKLISSVSGFKRNDKEGQPRRNLFTYYSVREGYKVVPDMQKHFLLFALVNHLSEQDDHIEK |
| 4764 | QQQSDELGKGLFFHRIASTFLNESGIFNKMQFYTYQSNRLKEKRGELKHEKDTFTWIEPFQGNSYFTLNGH |
| | KGVISEDQLKELCYTILIEKQNVDSLEGKIIQFLKKFQNVSSKQQVDEDELLKREYFPANYFGRAGTGTLKE |
| | KILNRLDKRMDPTSKVTDKAYDKMIEVMEFINMCLPSDEKLRQKDYRRYLKMVRFWNKEKHNIKREFDS |
| | KKWTRFLPTELWNKRNLEEAYQLARKENKKKLEDMRNQVRSLKENDLEKYQQINYVNDLENLRLLSQEL |
| | GVKWQEKDWVEYSGQIKKQISDNQKLTIMKQRITAELKKMHGIENLNLRISIDTNKSRQTVMNRIALPKGF |
| | VKNHIQQNSSEKISKRIREDYCKIELSGKYEELSRQFFDKKNFDKMTLINGLCEKNKLIAFMVIYLLERLGFE |
| | LKEKTKLGELKQTRMTYKISDKVKEDIPLSYYPKLVYAMNRKYVDNIDSYAFAAYESKKAILDKVDIIEKQ |
| | RMEFIKQVLCFEEYIFENRIIEKSKFNDEETHISFTQIHDELIKKGRDTEKLSKLKHARNKALHGEIPDGTSFE |
| | KAKLLINEIKK |
| IMG_ | MKFKNLRNDNEGIALAIGFNLAVANLEYFYNHIHGKKNVDISKIISANRTHNANEKLADFIWHEAKFKLFY |
| 3300026534 | KTPEKTLQNNLTIIIKRLNNLRNYYSHFCHSDEVLKIGKDEVDLITKLFNNALAFEKDYSEEIILFENNSFTKE |
| SEQ ID NO: | GVIWFVALFLYKFQAKQLFPHISGFKKNTGLYKSKHKLFSFYCTDFKNTNVKNDDPDFEHFLQIIQYLNRNP |
| 4765 | FANENEDNFRKTNMFIHFVVKFFDDFNVFPEIEFLKKERYNNLNEDDTKNEISENNVYQYLINRNNIFFEWN |
| | IDNFNYKIEDNNQTKKLKGIIGYQTLVYLVYAAFLKPNYSIISDEVKKFYTTYNKLLEDINNFNNYLKDIEYV |
| | GEQNLPKVIRAKIEDTNDKVTLKQKVLNRIEFILFQLNNNQNGLNRNGKPLRPYDKIAIVTDYINSELTDSQ |
| | KNENIKKSKTKFNAVKYKEIMSYIRYYKRDKETLIKILKNERWKFKSKIIKLLEDNSSLEELFLSVTDLKKGK |
| | YTDLKKEVENNKSNISEVAKELNIKKTKERKIDNSYLTGIKSNGIALPAEFIKRKLLKINKNIFN |
| IMG_ | MKFKNLRNDNEGIALAIGFNLAVANLEYFYNHIHGKKNVDISKIISANRTHNANEKLADFIWHEAKFKLFY |
| 3300026534_2 | KTPEKTLQNNLTIIIKRLNNLRNYYSHFCHSDEVLKIGKDEVDLITKLFNNALAFEKDYSEEIILFENNSFTKE |
| SEQ ID NO: | GVIWFVALFLYKFQAKQLFPHISGFKKNTGLYKSKHKLFSFYCTDFKNTNVKNDDPDFEHFLQIIQYLNRNP |
| 4766 | FANENEDNFRKTNMFIHFVVKFFDDFNVFPEIEFLKKERYNNLNEDDTKNEISENNVYQYLINRNNIFFEWN |
| | IDNFNYKIEDNNQTKKLKGIIGYQTLVYLVYAAFLKPNYSIISDEVKKFYTTYNKLLEDINNFNNYLKDIEYV |
| | GEQNLPKVIRAKIEDTNDKVTLKQKVLNRIEFILFQLNNNQNGLNRNGKPLRPYDKIAIVTDYINSELTDSQ |
| | KNENIKKSKTKFNAVKYKEIMSYIRYYKRDKETLIKILKNERWKFKSKIIKLLEDNSSLEELFLSVTDLKKGK |
| | YTDLKKEVENNKSNISEVAKELNIKKTKERKIDNSYLTGIKSNGIALPAEFIKRKLLKINKNIFN |
| IMG_ | MASAAPFVQNNSGYKRESRPPRTPTQKEVFTGGKVDYTELTVFFNIAYFRLAGVIHHLMGKPYEFEIDDKG |
| 3300011249 | VTKIIGKRAIEKVYNEESITEWQTDKKVLAGLNDYLFKGFKKAKNSSGYEMDEKDEKLVIFMVKKFKSIRN |
| SEQ ID NO: | FHSHYYHDNTVLVFPKNEKETIIKLHNEAISALMATQPKEVEKYVESISKNPFFKEHDREFYMTREGKIFLLS |
| 4767 | FFLTRSEMARLLQQCKGFKRNDTAEFKIKQSVYRHFTHRDGAARQHYGQEENMLNSLEPNDKKDILNARQ |
| | AFKIISYLNDVPPEANDTELFPLFLENKPVVLVEEFRTFCNAHSIFSEITIVPLVKTVKDAENDKLTKDITLDN |
| | WLVVKMNDYDIQITKTTFHKLILDSLRRNDSGKLVEAQLLKFVDERNYLYELVKTFKPKGALSENNKLTLT |
| | DELDEYYRFKLRSDFLQKTMGKWLEGKEESPKQPVPRNYRYITESEKFVNRIKLEPIEVNYYDFYFEADEKP |
| | RAADLFMKYAVQYLIDFEKVRDWYFMVEHFEFVEETKTVMEYGVPVVNKFMVNKRIISYANTIEESKRLS |
| | LTPDNQIVVGFYTDTENKTVPPKNKFLLGARALKNLLIALHQSNDINPFFDDIVTDLNQIRKGVQPDDLTTL |
| | KNYDIPASYKYAINNESIDIEAQKAKAQKRIETLVTELRTLLGNTAPKMSRADKNRQIMRCYKYFDWKYA |
| | NSEFKFLRQDEYQQVSVYHYSLEKRRGKDLERGDLSFLLKGAIDHMPEVVKELLRASSHIDKLLESTIEKTI |
| | NKL |
| IMG_ | MASAAPFVQNNSGYKRESRPPRTPTQKEVFTGGKVDYTELTVFFNIAYFRLAGVIHHLMGKPYEFEIDDKG |
| 3300015024 | VTKIIGKRAIEKVYNEESITEWQTDKKVLAGLNDYLFKGFKKAKNSSGYEMDEKDEKLVIFMVKKFKSIRN |
| SEQ ID NO: | FHSHYYHDNTVLVFPKNEKETIIKLHNEAISALMATQPKEVEKYVESISKNPFFKEHDREFYMTREGKIFLLS |
| 4768 | FFLTRSEMARLLQQCKGFKRNDTAEFKIKQSVYRHFTHRDGAARQHYGQEENMLNSLEPNDKKDILNARQ |
| | AFKIISYLNDVPPEANDTELFPLFLENKPVVLVEEFRTFCNAHSIFSEITIVPLVKTVKMQKTTN |

In some embodiments, the small Cas proteins are small Cas 13c. Examples of small Cas13c are shown in Table 4 below.

TABLE 4

| Accession | |
|---|---|
| No. | Sequences |
| GCA_ | MKKLKNPSNRNSLPSIIISKFDSSKIYEIKVKYEKLARLDRLEIGDMSLDENLNILFKKVNFNGIDLEILNPL |
| 004116325.1_ | LLDFDSYTISGKLQKNSTNKTILTLKKDGKIIKYNVLEKDNKYFKNGKEFVIPKDVKEEGKRLVNDKFLL |
| ASM411632v1_ | TIEDKKREENSLPKKRKKETQRDILKDETIEIYKRISSNSNIKSEDIYRIKRYMLFRSDMMFFYTFIDNFFYC |
| genomic | LYKNKNEQLWNTNFKEKENLGKFIEFTLNDTLKNPRNGILKSYSKDLKVVQEDFVKIKDIFEKIRHALAH |
| SEQ ID NO: | FDFTFIDNLLSNNIEFDFNIKLLNIVIEDSQDLYYEAKKEFIEDEKMDILDEKDISIKKLYTFYSKIDIKKPAF |
| 4769 | NKLINSFLIKDGVENSKLKEYIKEKYNCHYFIDIHDNKEYKKIYNEHKKLISENQNLQLNSKENGQKIKIN |
| | NDRLEELKGKMNELTKANSLKRLEFKLRLAFGFIKVEYNIFKDFKNNFSEDIKKDMNIDLEKIKSYLDTS |
| | YSNNQFFNYKVYNKKTKQKDIDKDIFDDIEKETLKELVENDSLLKIILLFYIFTPKELKGEFLGFIKKFYHD |
| | TKNIDKDTKDKEEPLEQIKQEVPLKLKILEKNLTILTIFNYSISLNIEYDKNNNSFYERGNKFKKIYKDLKIS |
| | HNQEEFDKSLLAPLLKYYMNLYKLLNDFEIYLLLKYKNKDNLNKESLNKLINDEQLKHNDHYNFTTLLS |
| | EYFNFDPKKNKKYETLTILRNSISHQKIDNLIYNLDKNKILEQRVKIVELIKEQRDIKETLKFDPINDFTMK |
| | TVQLLKSLENQSEKRDKIEEILKQQDLSANDFYNIYKLKGVESIKKELFIRLGKTKIEEKIQEDIAKGSI |
| GCA_ | LNSIEKIKKPSNRNSIPSIIISDYDENKIKEIKVKYLKLARLDKITIQDMEIRDNIVEFKKILLNGIEHTIKDNQ |
| 002837275.1_ | KIEFDNYEITAYVRASKQRRDGKITQAKYVVTITDKYLRDNEKEKRFKSTERELPNDTLLMRYKQISGFD |
| ASM283727v1_ | TLTSKDIYKIKRYIDFKNEMLFYFQFIEEFFSPLLPKGTNFYSLNIEQNKDKVVKYIVYRLNDDFKNQSLN |
| genomic | QFIKKTDTIKYDFLKIQKILSDFRHALAHFDFDFIQKFFDDELDKNRFDISTISLIKTMLQEKEEKYYQEKN |
| SEQ ID NO: | NYIEDSDTLTLFDEKESNFSKIHNFYIKISQKKPAFNKLINSFLSKDGVPNEELKSYLATKKIDFFEDIHSNK |
| 4770 | EYKKIYIKHKNLVVEKQKEESQEKPNGQKLKNYNDELQKLKDEMNKITKQNSLNRLEVKLRLAFGFIAN |
| | EYNYNFKNFNDKFTLDVKKEQKIKVFKNSSNEKLKEYFESTFIEKRFFHFCVKFFNKKTKKEETKQKNIF |
| | NLIENETLEELVKESPLLQIITLLYLFIPKELQGEFVGFILKIYHHTKNITNDTKEDEKSIEDTQNSFSLKLKIL |
| | AKNLRGLQLFNYSLSHNTLYNTKEHFFYEKGNRWQSVYKSLEISHNQDEFDIHLVIPVIKYYINLNKLIGD |
| | FEIYALLTYADKNSITEKLSDITKRDDLKFRGYYNFSTLLFKTFMINTNYEQNQKSTQYIKQTRNDIAHQN |
| | IENMLKAFENNEIFAQREEIVNYLQKEHKMQEILHYNPINDFTMKTVQYLKSLNIHSQKESKIADIHKKES |
| | LVPNDYYLIYKLKVIELLKQKVIEAIGETKDEEKIKNAIAKEEQIKKGYNK |
| GCA_ | LNSIEKIKKPSNRNSIPSIIISDYDENKIKEIKVKYLKLARLDKITIQDMEIRDNIVEFKKILLNGIEHTIKDNQ |
| 003346755.1_ | KIEFDNYEITAYVRASKQRRDGKITQAKYVVTITDKYLRDNEKEKRFKSTERELPNDTLLMRYKQISGFD |
| ASM334675v1_ | TLTSKDIYKIKRYIDFKNEMLFYFQFIEEFFSPLLPKGTNFYSLNIEQNKDKVVKYIVYRLNDDFKNQSLN |
| genomic | QFIKKTDTIKYDFLKIQKILSDFRHALAHFDFDFIQKFFDDELDKNRFDISTISLIKTMLQEKEEKYYQEKN |
| SEQ ID NO: | NYIEDSDTLTLFDEKESNFSKIHNFYIKISQKKPAFNKLINSFLSKDGVPNEELKSYLATKKIDFFEDIHSNK |
| 4771 | EYKKIYIKHKNLVVEKQKEESQEKPNGQKLKNYNDELQKLKDEMNKITKQNSLNRLEVKLRLAFGFIAN |
| | EYNYNFKNFNDKFTLDVKKEQKIKVFKNSSNEKLKEYFESTFIEKRFFHFCVKFFNKKTKKEETKQKNIF |
| | NLIENETLEELVKESPLLQIITLLYLFIPKELQGEFVGFILKIYHHTKNITNDTKEDEKSIEDTQNSFSLKLKIL |
| | AKNLRGLQLFNYSLSHNTLYNTKEHFFYEKGNRWQSVYKSLEISHNQDEFDIHLVIPVIKYYINLNKLIGD |
| | FEIYALLTYADKNSITEKLSDITKRDDLKFRGYYNFSTLLFKTFMINTNYEQNQKSTQYIKQTRNDIAHQN |
| | IENMLKAFENNEIFAQREEIVNYLQKEHKMQEILHYNPINDFTMKTVQYLKSLNIHSQKESKIADIHKKES |
| | LVPNDYYLIYKLKVIELLKQKVIEAIGETKDEEKIKNAIAKEEQIKKGYNK |
| GCF_ | LNSIEKIKKPSNRNSIPSIIISDYDENKIKEIKVKYLKLARLDKITIQDMEIRDNIVEFKKILLNGIEHTIKDNQ |
| 003346755.1_ | KIEFDNYEITAYVRASKQRRDGKITQAKYVVTITDKYLRDNEKEKRFKSTERELPNDTLLMRYKQISGFD |
| ASM334675v1_ | TLTSKDIYKIKRYIDFKNEMLFYFQFIEEFFSPLLPKGTNFYSLNIEQNKDKVVKYIVYRLNDDFKNQSLN |
| genomic | QFIKKTDTIKYDFLKIQKILSDFRHALAHFDFDFIQKFFDDELDKNRFDISTISLIKTMLQEKEEKYYQEKN |
| SEQ ID NO: | NYIEDSDTLTLFDEKESNFSKIHNFYIKISQKKPAFNKLINSFLSKDGVPNEELKSYLATKKIDFFEDIHSNK |
| 4772 | EYKKIYIKHKNLVVEKQKEESQEKPNGQKLKNYNDELQKLKDEMNKITKQNSLNRLEVKLRLAFGFIAN |
| | EYNYNFKNFNDKFTLDVKKEQKIKVFKNSSNEKLKEYFESTFIEKRFFHFCVKFFNKKTKKEETKQKNIF |
| | NLIENETLEELVKESPLLQIITLLYLFIPKELQGEFVGFILKIYHHTKNITNDTKEDEKSIEDTQNSFSLKLKIL |
| | AKNLRGLQLFNYSLSHNTLYNTKEHFFYEKGNRWQSVYKSLEISHNQDEFDIHLVIPVIKYYINLNKLIGD |
| | FEIYALLTYADKNSITEKLSDITKRDDLKFRGYYNFSTLLFKTFMINTNYEQNQKSTQYIKQTRNDIAHQN |
| | IENMLKAFENNEIFAQREEIVNYLQKEHKMQEILHYNPINDFTMKTVQYLKSLNIHSQKESKIADIHKKES |
| | LVPNDYYLIYKLKVIELLKQKVIEAIGETKDEEKIKNAIAKEEQIKKGYNK |
| IMG_ | MEKIKKPSNRNSIPSIIISDYDANKIKEIKVKYLKLARLDKITIQDMEIVDNIVEFKKILLNGVEHTIIDNQKI |
| 3300028602 | EFDNYEITGCIKPSNKRRDGRISQAKYVVTITDKYLRENEKEKRFKSTERELPNNTLLSRYKQISGFDTLTS |
| SEQ ID NO: | KDIYKIKRYIDFKNEMLFYFQFIEEFFNPLLPKGKNFYDLNIEQNKDKVAKFIVYRLNDDFKNKSLNSYIT |
| 4773 | DTCMIINDFKKIQKILSDFRHALAHFDFDFIQKFFDDQLDKNKFDINTISLIETLLDQKEEKNYQEKNNYID |
| | DNDILTIFDEKGSKFSKLHNFYTKISQKKPAFNKLINSFLSQDGVPNEEFKSYLVTKKLDFFEDIHSNKEYK |
| | KIYIQHKNLVIKKQKEESQEKPDGQKLKNYNDELQKLKDEMNTITKQNSLNRLEVKLRLAFGFIANEYN |
| | YNFKNFNDEFTNDVKNEQKIKAFKNSSNEKLKEYFESTFIEKRFFHFSVNFFNKKTKKEETKQKNIFNSIE |
| | NETLEELVKESPLLQIITLLYLFIPRELQGEFVGFILKIYHHTKNITSDTKEDEISIEDAQNSFSLKFKILAKNL |
| | RGLQLFHYSLSHNTLYNNKQCFFYEKGNRWQSVYKSFQISHNQDEFDIHLVIPVIKYYINLNKLMGDFEI |
| | YALLKYADKNSITVKLSDITSRDDLKYNGHYNFATLLFKTFGIDTNYKQNKVSIQNIKKTRNNLAHQNIE |
| | NMLKAFENSEIFAQREEIVNYLQTEHRMQEVLHYNPINDFTMKTVQYLKSLSVHSQKEGKIADIHKKESL |
| | VPNDYYLIYKLKAIELLKQKVIEVIGESEDEKKIKNAIAKEEQIKKGNN |
| IMG_ | LQTLVQDNPLLQIITLLYLFIPKELQGDFIGFILHIYHQTKNITSDTKEDEISLEESQNSFALKLKVLAKSLRG |
| 3300000233 | LQLFNYSLSHDTLYNTKEHFFYEKGNRWKNIYKALGISHNTEEFDIHLVTPIIKYHINLYKLIGDFEIYALL |
| SEQ ID NO: | TFTKKSRSHETLSVISKSDALKFKENYNFSTLLSKAFRIDVNNKNNPPYIQTLKQIRNDISHQNIEKMMTAF |
| 4774 | EQNDIFEQRKEIIIYLQTDHQEMQKLLHYNPVNDFTMKTVQYCIMLDKYKMGVADNDEKIENRADLIIK |
| | NLKKETPNDYYLIYKLKAIELLKQKMIEAIGETEQEKKIRKAIAK |
| IMG_ | MSQLKNPSNKNSLPRIIISDFNEIKINEIKIKYHKLDRLDKIIVKEMEIINNKIFFKKILFNNQIKDINSENIELE |
| 3300019761 | NYILAGEVKPSNTKIILNRDGKEKSFIVYDGFTFKYKPNDKRISETKTNAKYILTIKDKTRHRESSTQRDIL |
| SEQ ID NO: | KSSIIETYKQISGFENITSKDIYTIKRYIDFKNEMMFYYTFIDDFFFPITGKNKQDKKNNFYNYKIKENAKKF |
| 4775 | ISLINYRINDDFKNKNGILYDYLSNKEEIIINDFIHIQTILKDVRHAIAHFNFDFIQKLFDNEQAFNSKFDGIEI |
| | LNILFNQKQEKYFEAQTNYIEEETIKILDEKELSFKKLHSFYSQICQKKPAFNKLINSFIIQDGIENKELKDYI |
| | SQKYNSKFDYYLDIHTCKIYKDIYNQHKKFVADKQFLENQKTDGQKIKKLNDQINQLKTKMNNLTKKN |
| | SLKRLEIKFRLAFGFIFTEYQTFKNFNERFIEDIKANKYSTKIELLDYGKIKEYISITHEEKRFFNYKTFNKK |
| | TNKNINKTIFQSLEKETFENLVKNDNLIKMMFLFQLLLPRELKGEFLGFILKIYHDLKNIDNDTKPDEKSLS |
| | ELNISTALKLKILVKNIRQINLFNYTISNNTKYEEKEKRFYEEGNQWKDIYKKLYISHDFDIFDIHLIIPIIKY |
| | NINLYKLIGDFEVYLLLKYLERNTNYKTLDKLIEAEELKYKGYYNFTTLLSKAINIALNDKEYHNITHLRN |
| | NTSHQDIQNIISSFKNNKLLEQRENIIELISKESLKKKLHFDPINDFTMKTLQLLKSLEVHSDKSEKIENLLK |
| | KEPLLPNDVYLLYKLKGIEFIKKELISNIGITKYEEKIQEKIAKGVEK |
| IMG_ | MVKNPANRHALPKVIISEVDNNNILEFKIKYEKLARLDKVEVKSMHFDNNKQVVFDEVVINGGLIEPTYE |
| 3300021977 | DKHKKLVVTAGEKSYSIVGQKVGGKPRLLEDRVSKTKVQLELTNYVEDKEGKKRVSKTERELIVADNIE |
| SEQ ID NO: | LYSQIVGREVKTTKEIYLIKRFLEYRSDLLFYYGFVDNFFKVAGNGKELWKIDFTNSDSLHLIEYFKFSIND |
| 4776 | NLKNDENYLKNYVSDNTKIENDLVKCQNNFNSLRHALMHFDYDFFEKLFNGEDVGFDFDIEFLNIMIDK |
| | VDKLNIDTKKEFIDDEEVTLFGEALSLKKLYGLFSHIAINRVAFNKLINSFIIEDGIENKELKDFFNNKKESQ |
| | AYEIDIHSNAEYKALYVQHKKLVMATSAMTDGDEIAKKNQEISDLKEKMKVITKENSLARLEHKLRLAF |
| | GFIYTEYKDYKTFKKHFDQDIKGAKYKGLNVEKLKEYYETTLKNSKPKTDEKLEDVAKKIDKLSLKELI |
| | DDDTLLKFVLLLFIFMPQELKGDFLGFIKKYYHDKKHIDQDTKDKDTEIEELSTGLKLKVLDKNIRSLSIL |
| | KHSFSFQVKYNRKDKNFYEDGNLHGKFYKKLSISHNQEEFNKSVYAPLFRYYSALYKLINDFEIYALAQ |
| | HVENHETLADQVNKSQFIQKSYFNFRKLLDNTDSISQSSSYNTLIVMRNDISHLSYEPLFNYPLDERKSYK |
| | KKTQKGVKTFHVELLYISRAKIIELISLQTDMKKLLGYDAVNDFNMKVVHLRKRLSVYANKEESIRKMQ |
| | ADAKTPNDFYNIYKVKGVESINQHLLKVIGVTEAEKSIEKQINEGNKKHNT |
| IMG_ | MIKNPSNRYALPKVIISKIDNQNILEFKIKYKKLSKLDIVKVKSMHYDDRAIIFDEVIVNDGLIDVEYRDNH |
| 3300026521 | KTIFVKVGNKSYSISGQKVGGKERLLENRVSKTKVQLELKDKATNRVSKTERELIVDDNIKIYSQIVGRD |
| SEQ ID NO: | VKTTKDIYLIKRFLAYRSDLLFYYGFVNNFFHVANNRSEFWKIDFNDSNNSKLIEYFKFTINDHLKNDEN |
| 4777 | YLKDYISDNEKLKNDLIKVKNSFEKIRHALMHFDYDFFVKLFNGEDVGLELDIEFLDIMIDKLDKLNIDTK |
| | KEFIDDEKITIFGEELSLAKLYRFYAHTAINRVAFNKLINSFIIENGVENQSLKEYFNQQAGGIAYEIDIHQN |
| | REYKNLYNEHKKLVSRVLSISDGQEIAILNQKIAKLKDQMKQITKANSIKRLEYKLRLALGFIYTEYENYE |
| | EFKNNFDTDIKNGRFTPKDNDGNKRAFDSRELEQLKGYYEATIQTQKPKTDEKIEEVSKKIDRLSLKSLIA |
| | DDILLKFILLMFTFMPQELKGEFLGFIKKYYHDTKHIDQDTISDSDDTIETLSIGLKLKILDKNIRSLSILKHS |
| | LSFQTKYNKKDRNYYEDGNIHGKFFKKLGISHNQEEFNKSVYAPLFRYYSALYKLINDFEIYTLSLHIVGS |
| | ETLTDQVNKSQFLSGRYFNFRKLLTQSYHINNNSTHSTIFNAVINMRNDISHLSYEPLFDCPLNGKKSYKR |
| | KIRNQFKTINIKPLVESRKIIIDFITLQTDMQKVLGYDAVNDFTMKIVQLRTRLKAYANKEQTIQKMITEA |
| | KTPNDFYNIYKVQGVEEINKYLLEVIGETQAEKEIREKIERGNIANF |
| IMG_ | MIKNPSNRHSLPKVIISEVDHEKILEFKIKYEKLARLDRFEVKAMHYEGKEIVFDEVLVNGGLIEVEYQDD |
| 3300028030 | NKTLFVKVGEKSYSIRGKKVGGKQRLLEDRVSKTKVQLELSDGVVDNKGNLRKSRTERELIVADNIKLY |
| SEQ ID NO: | SQIVGREVTTTKEIYLVKRFLAYRSDLLFYYSFVDNFFKVAGNEKELWKINFDDATSAQFMGYIPFMVND |
| 4778 | NLKNDNAYLKDYVRNDVQIKDDLKKVQTIFSALRHTLLHFNYEFFEKLFNGEDVGFDFDIGFLNLLIENI |
| | DKLNIDAKKEFIDNEKIRLFGENLSLAKVYRLYSDICVNRVGFNKFINSMLIKDGVENQVLKAEFNRKFG |
| | GNAYTIDIHSNQEYKRIYNEHKKLVIKVSTLKDGQAIRRGNKKISELKEQMKSMTKKNSLARLECKMRL |
| | AFGFLYGEYNNYKAFKNNFDTNIKNSQFDVNDVEKSKAYFLSTYERRKPRTREKLEKVAKDIESLELKT |
| | VIANDTLLKFILLMFVFMPQELKGDFLGFVKKYYHDVHSIDDDTKEQEEDVVEAMSTSLKLKILGRNIRS |
| | LTLFKYALSSQVNYNSTDNIFYVEGNRYGKIYKKLGISHNQEEFDKTLVVPLLRYYSSLFKLMNDFEIYSL |
| | AKANPTAVSLQELVDDETSPYKQGNYFNFNKMLRDIYGLTSDEIKSGQVVFMRNKIAHFDTEVLLSKPL |
| | LGQTKMNLQRKDIVSFIEARGDIKELLGYDAINDFRMKVIHLRTKMRVYSDKLQTMMDLLRNAKTPND |
| | FYNVYKVKGVESINKHLLEVLAQTAEERTVEKQIRDGNEKYDL |
| IMG_ | MIKNPSNRHSLPKVIISEVDHEKILEFKIKYEKLARLDRFEVKAMHYEGKEIVFDEVLVNGGLIEVEYQDD |
| 3300028030_2 | NKTLFVKVGEKSYSIRGKKVGGKQRLLEDRVSKTKVQLELSDGVVDNKGNLRKSRTERELIVADNIKLY |
| SEQ ID NO: | SQIVGREVTTTKEIYLVKRFLAYRSDLLFYYSFVDNFFKVAGNEKELWKINFDDATSAQFMGYIPFMVND |
| 4779 | NLKNDNAYLKDYVRNDVQIKDDLKKVQTIFSALRHTLLHFNYEFFEKLFNGEDVGFDFDIGFLNLLIENI |
| | DKLNIDAKKEFIDNEKIRLFGENLSLAKVYRLYSDICVNRVGFNKFINSMLIKDGVENQVLKAEFNRKFG |
| | GNAYTIDIHSNQEYKRIYNEHKKLVIKVSTLKDGQAIRRGNKKISELKEQMKSMTKKNSLARLECKMRL |
| | AFGFLYGEYNNYKAFKNNFDTNIKNSQFDVNDVEKSKAYFLSTYERRKPRTREKLEKVAKDIESLELKT |
| | VIANDTLLKFILLMFVFMPQELKGDFLGFVKKYYHDVHSIDDDTKEQEEDVVEAMSTSLKLKILGRNIRS |
| | LTLFKYALSSQVNYNSTDNIFYVEGNRYGKIYKKLGISHNQEEFDKTLVVPLLRYYSSLFKLMNDFEIYSL |
| | AKANPTAVSLQELVDDETSPYKQGNYFNFNKMLRDIYGLTSDEIKSGQVVFMRNKIAHFDTEVLLSKPL |
| | LGQTKMNLQRKDIVSFIEARGDIKELLGYDAINDFRMKVIHLRTKMRVYSDKLQTMMDLLRNAKTPND |
| | FYNVYKVKGVESINKHLLEVLAQTAEERTVEKQIRDGNEKYDL |
| IMG_ | MMTKKPANRHALPKVIISEVDNTNILEFKIKYEKLARLDRVEVKAMHYEDGRIIFDEVVVNGGLIEVEYQ |
| 3300026544 | DDHKTLFVQVGEKSYSISGQKVGGKQRLLEDRVSKTKVQLELSDGSSERVSRTERELIVADNIKLYSQIV |
| SEQ ID NO: | GHEVKTTKEIYLAKRFLGYRSDLLFYYGFVDNFFRESKNLKYGKQPVELWEDKFQVNDKLTAYTKFMF |
| 4780 | NDDLQNSESYLKEYVKDNHKIKNDLESARDIFATFRHNLMHFNYSFFTRLFNGEDVKIKNLQTKKFESLS |
| | DVLRNVEFLNKVIQSIDKLNIDTRKEFIDKEKITLFNEELDLQQLYGFFAYTAINRVAFNKLINSFIIKDGIE |
| | NEQLKEYFNQRVDGTAYEIDIHQNREYKELYKKHKNLVSKVSTLSDGKEIARGNTEISVLKEQMNKITK |
| | ANSLKRLEHKLRLAFGFIYTEYGSYKAFVSRFNEDTKRKKIKNVEFEKIGVEKQKEYYESTFTSNNKDKL |
| | GELIQEYEKLSLNDLIENDTFLKVILLLFIFMPKEVKGDFLGFIKKYYHDTKHIEEDTKEKDEGFTNTLPIG |
| | LKLKIVERNIAKLSVLKHSLSLKVKYNRGQYEEDNTYRKVFKKLNISHNQEEFHKSMFSPLLRYYASLYK |
| | LINDFEIYTLSHYITDKYSTLNKVIASEQFHYRYGWNREEKKGELVKTDNYTFSTLLSKKYGHKNSQEISE |
| | MRNKISHFDEKILFKFPLEEVSSVPKGKGKYKKDEPIKSLKEKREEIVSLMEKQTDMQKVLGYDAINDFR |
| | MKTVQFQTKLKVYSNKEETIKKMIVEAKTPNDYYNIYKVKGVEGINEHLLNVIGETEAEKSIQEQIAEGN |
| | KVNV |
| IMG_ | MTKKPSNRNSLPKVIINKVDESSILEFKIKYEKLARLDRFEVRSMRYDGDGRIIFDEVVANAGLLDVDYE |
| 3300021977_2 | DDNRTIVVKIENKAYNIYGKKVGGEKRLNGKISKAKVQLILTDSIRKNANDTHRHSLTERELINKNEVDL |
| SEQ ID NO: | YSKIAEREISTTKDIYLVKRFLAYRSDLLLYYAFINHYVRVNGNKKEFWKTEIDDKIIDYFIYTINDTLKNK |
| 4781 | EGYLEKYIVDRDQIKKDLEKIKQIFSHLRHKLMHYDFRFFTDLFDGKDVDIKVDNSIQKISELLDIEFLNIVI |
| | DKLEKLNIDAKKEFIDDEKITLFGQEIELKKLYSLYAHTSINRVAFNKLINSFLIKDGVENKELKEYFNAHN |
| | QGKESYYIDIHQNQEYKKLYIEHKNLVAKLSATTDGKEIAKINRELADKKEQMKQITKANSLKRLEYKL |
| | RLAFGFIYTEYKDYERFKNSFDTDTKKKKFDAIDNAKIIEYFEATNKAKKIEKLEEILKGIDKLSLKTLIQD |
| | DILLKFLLLFFTFLPQEIKGEFLGFIKKYYHDITSLDEDTKDKDDEITELPRSLKLKIFSKNIRKLSILKHSLS |
| | YQIKYNKKESSYYEAGNVFNKMFKKQAISHNLEEFGKSIYLPMLKYYSALYKLINDFEIYALYKDMDTS |
| | ETLSQQVDKQEYKRNEYFNFETLLRKKFGNDIEKVLVTYRNKIAHLDFNFLYDKPINKFISLYKSREKIVN |
| | YIKNHDIQAVLKYDAVNDFVMKVIQLRTKLKVYADKEQTIESMIQNTQNPNGFYNIYKVKAVENINRHL |
| | LKVIGYTESEKAVEEKIRAGNTSKS |
| IMG_ | MLKHKRKNKNSLARVVLSNYDSNNIYEIKIKYEKLAKLDKINIIEMDYDADNNVMFKKVLFNNKEIDLS |
| 3300026382 | HKDKTKINIELDNKKYNISAKKQIGKTHLVVRNKQTSKISRIKKIQDTYYRGKDVFILDNNIEILDKKQTK |
| SEQ ID NO: | DKFIVTLNDITNNKTTSTEAELIDDTKDIFKKISAKKDLKSSDIYKIKRFISIRSNFSFYYTFVDNYFKIFHAK |
| 4782 | KDKNKEELYKIKFKDEINIKPYLENILDNMKNKNGILYNYANDRKKVLNDLRNIQYVFKEFRHKLAHFD |
| | YNFLDNFFSNSVEEKYKQKVNEIKLLDILLDNIDSLNVVPKQNYIEDETISVFDAKDIKLKRLYTYYIKLTI |
| | NYPGFKKLINSFFIQDGIENQELKEYINNKEKDTQVLKELDNKAYYMDISQYRKYKNIYNKHKELVSEKE |
| | LSSDGKKINSLNQKINKLKIDMKNITKPNALNRLIYRLRVAFGFIYKEYATINNFNKSFLQDTKTKRFENIS |
| | QQDIKSYLDISYQDKGKFFVKSKKTFKNKTTVKYTFEDLDLTLNEIITQDDIFVKVIFLFSIFMPKELNGDF |
| | FGFINMYYHKMKNISYDTKDIDMLDTISQNMKLKILEQNIKKTYVFKYYLDLDSSIYSKLVQNIKITEDID |
| | SKKYLYAKIFKYYQHLYKLISDVEIYLLYKYNSKENLSITIDKDELKHRGYYNFQSLLIKNNINKDDAYW |
| | SIVNMRNNLSHQNIDELVGHFCKGCLRKSTTDIAELWLRKDILTITNEIINKIESFKDIKITLGYDCVNDFT |
| | QKVKQYKQKLKASNERLAKKIEEKQNQVVDEKNKEELEKNILNMKNIQKINRYILDIL |
| IMG_ | MLKHKRKNKNSLARVVLSNYDSNNIYEIKIKYEKLAKLDKINIIEMDYDADNNVMFKKVLFNNKEIDLS |
| 3300026382_2 | HKDKTKINIELDNKKYNISAKKQIGKTHLVVRNKQTSKISRIKKIQDTYYRGKDVFILDNNIEILDKKQTK |
| SEQ ID NO: | DKFIVTLNDITNNKTTSTEAELIDDTKDIFKKISAKKDLKSSDIYKIKRFISIRSNFSFYYTFVDNYFKIFHAK |
| 4783 | KDKNKEELYKIKFKDEINIKPYLENILDNMKNKNGILYNYANDRKKVLNDLRNIQYVFKEFRHKLAHFD |
| | YNFLDNFFSNSVEEKYKQKVNEIKLLDILLDNIDSLNVVPKQNYIEDETISVFDAKDIKLKRLYTYYIKLTI |
| | NYPGFKKLINSFFIQDGIENQELKEYINNKEKDTQVLKELDNKAYYMDISQYRKYKNIYNKHKELVSEKE |
| | LSSDGKKINSLNQKINKLKIDMKNITKPNALNRLIYRLRVAFGFIYKEYATINNFNKSFLQDTKTKRFENIS |
| | QQDIKSYLDISYQDKGKFFVKSKKTFKNKTTVKYTFEDLDLTLNEIITQDDIFVKVIFLFSIFMPKELNGDF |
| | FGFINMYYHKMKNISYDTKDIDMLDTISQNMKLKILEQNIKKTYVFKYYLDLDSSIYSKLVQNIKITEDID |
| | SKKYLYAKIFKYYQHLYKLISDVEIYLLYKYNSKENLSITIDKDELKHRGYYNFQSLLIKNNINKDDAYW |
| | SIVNMRNNLSHQNIDELVGHFCKGCLRKSTTDIAELWLRKDILTITNEIINKIESFKDIKITLGYDCVNDFT |
| | QKVKQYKQKLKASNERLAKKIEEKQNQVVDEKNKEELEKNILNMKNIQKINRYILDIL |
| IMG_ | MLKHKRKNKNSLARVVLSNYDSNNIYEIKIKYEKLAKLDKINIIEMDYDADNNVMFKKVLFNNKEIDLS |
| 3300026512 | HKDKTKINIELDNKKYNISAKKQIGKTHLVVRDKQTSKISRIKKIQDTYYRGKDVFILDNNIEILDKKQTK |
| SEQ ID NO: | DKFIVTLNDITNDKTTSTEAELIDDTKDIFKKISAKKDLKSSDIYKIKRFISIRSNFSFYYTFVDNYFKIFHAK |
| 4784 | KDKNKEELYKIKFKDEINIKPYLENILDNMKNKNGILYDYADDREKVLNDLKNIQYVFTEFRHKLAHFD |
| | YNFLDNFFSNSVTDQYKQKVNEIKLLDILLDNIDSLNVVPKQNYIEDETISVFDAKDIKLKRLYTYYIKLTI |
| | NYPGFKKLINSFFIQDGIENQELKEYINNKEKDTQVLKELDNKAYYMDISQYRKYKNIYNKHKELVSEKE |
| | LSSDGQKINSLNQKINKLKIEMKNITKPNALNRLIYRLRVAFGFIYKEYATINNFNKSFLQDTKIKRFENIS |
| | QQDIKNYLDISYQDKGKFFVKSKKTFKNKTTIKYTFEDLDLTLNEIITQDDIFVKVIFLFSIFMPKELNGDFF |
| | GFINMYYHKMKNISYDTKDIDMLDTISQNMKLKILEQNIKKTYVFKYYLDLDSSIYSKLVQNIKITEDIDS |
| | KKYLYAKIFKYYQHLYKLISDVEIYLLYKYNSKENLSITIDKDELKHRGYYNFQSLLIKNNINKDDAYWSI |
| | VNMRNNLSHQNIDELVGHFCKGCLRKSTTDIAELWLRKDILTITNEIINKIESFKDIKITLGYDCVNDFTQK |
| | VKQYKQKLKASNERLAKKIEEKQNQVVDEKNKEELEKKILNMKNIQKINRYILDIL |
| IMG_ | MLKHKRKNKNSLARVVLSNYDSNNIYEIKIKYEKLAKLDKINIIEMDYDADNNVMFKKVLFNNKEIDLS |
| 3300026512_2 | HKDKTKINIELDNKKYNISAKKQIGKTHLVVRDKQTSKISRIKKIQDTYYRGKDVFILDNNIEILDKKQTK |
| SEQ ID NO: | DKFIVTLNDITNDKTTSTEAELIDDTKDIFKKISAKKDLKSSDIYKIKRFISIRSNFSFYYTFVDNYFKIFHAK |
| 4785 | KDKNKEELYKIKFKDEINIKPYLENILDNMKNKNGILYDYADDREKVLNDLKNIQYVFTEFRHKLAHFD |
| | YNFLDNFFSNSVTDQYKQKVNEIKLLDILLDNIDSLNVVPKQNYIEDETISVFDAKDIKLKRLYTYYIKLTI |
| | NYPGFKKLINSFFIQDGIENQELKEYINNKEKDTQVLKELDNKAYYMDISQYRKYKNIYNKHKELVSEKE |
| | LSSDGQKINSLNQKINKLKIEMKNITKPNALNRLIYRLRVAFGFIYKEYATINNFNKSFLQDTKIKRFENIS |
| | QQDIKNYLDISYQDKGKFFVKSKKTFKNKTTIKYTFEDLDLTLNEIITQDDIFVKVIFLFSIFMPKELNGDFF |
| | GFINMYYHKMKNISYDTKDIDMLDTISQNMKLKILEQNIKKTYVFKYYLDLDSSIYSKLVQNIKITEDIDS |
| | KKYLYAKIFKYYQHLYKLISDVEIYLLYKYNSKENLSITIDKDELKHRGYYNFQSLLIKNNINKDDAYWSI |
| | VNMRNNLSHQNIDELVGHFCKGCLRKSTTDIAELWLRKDILTITNEIINKIESFKDIKITLGYDCVNDFTQK |
| | VKQYKQKLKASNERLAKKIEEKQNQVVDEKNKEELEKKILNMKNIQKINRYILDIL |
| GCA_ | LTEKKSIIFKNKSSVEIVKKDIFSQTPDNMIRNYKITLKISEKNPRVVEAEIEDLMNSTILKDGRRSARREKS |
| 000242215.1_ | MTERKLIEEKVAENYSLLANCPMEEVDSIKIYKIKRFLTYRSNMLLYFASINSFLCEGIKGKDNETEEIWH |
| Fuso_necr_1_ | LKDNDVRKEKVKENFKNKLIQSTENYNSSLKNQIEEKEKLLRKESKKGAFYRTIIKKLQQERIKELSEKSL |
| 1_36S_V1_ | TEDCEKIIKLYSELRHPLMHYDYQYFENLFENKENSELTKNLNLDIFKSLPLVRKMKLNNKVNYLEDNDT |
| genomic | LFVLQKTKKAKTLYQIYDALCEQKNGFNKFINDFFVSDGEENTVFKQIINEKFQSEMEFLEKRISESEKKN |
| SEQ ID NO: | EKLKKKFDSMKAHFHNINSEDTKEAYFWDIHSSSNYKTKYNERKNLVNEYTELLGSSKEKKLLREEITQI |
| 4786 | NRKLLKLKQEMEEITKKNSLFRLEYKMKIAFGFLFCEFDGNISKFKDEFDASNQEKIIQYHKNGEKYLTYF |
| | LKEEEKEKFNLEKMQKIIQKTEEEDWLLPETKNNLFKFYLLTYLLLPYELKGDFLGFVKKHYYDIKNVDF |
| | MDENQNNIQVSQTVEKQEDYFYHKIRLFEKNTKKYEIVKYSIVPNEKLKQYFEDLGIDIKYLTGSVESGE |
| | KWLGENLGIDIKYLTVEQKSEVSEEKIKKFL |
| GCA_ | MENKGNNKKIDFDENYNILVAQIKEYFTKEIENYNNRIDNIIDKKELLKYSEKKEESEKNKKLEELNKLKS |
| 000158315.2_ | QKLKILTDEEIKADVIKIIKIFSDLRHSLMHYEYKYFENLFENKKNEELAELLNLNLFKNLTLLRQMKIEN |
| Fuso_ulc_ | KTNYLEGREEFNIIGKNIKAKEVLGHYNLLAEQKNGFNNFINSFFVQDGTENLEFKKLIDEHFVNAKKRL |
| ATCC49185_ | ERNIKKSKKLEKELEKMEQHYQRLNCAYVWDIHTSTTYKKLYNKRKSLIEEYNKQINEIKDKEVITAINV |
| V2_genomic | ELLRIKKEMEEITKSNSLFRLKYKMQIAYAFLEIEFGGNIAKFKDEFDCSKMEEVQKYLKKGVKYLKYYK |
| SEQ ID NO: | DKEAQKNYEFPFEEIFENKDTHNEEWLENTSENNLFKFYILTYLLLPMEFKGDFLGVVKKHYYDIKNVDF |
| 4787 | TDESEKELSQVQLDKMIGDSFFHKIRLFEKNTKRYEIIKYSILTSDEIKRYFRLLELDVPYFEYEKGTDEIGI |
| | FNKNIILTIFKYYQIIFRLYNDLEIHGLFNISSDLDKILRDLKSYGNKNINFREFLYVIKQNNNSSTEEEYRKI |
| | WENLEAKYLRLHLLTPEKEEIKTKTKEELEKLNEISNLRNGICHLNYKEIIEEILKTEISEKNKEATLNEKIR |
| | KVINFIKENELDKVELGFNFINDFFMKKEQFMFGQIKQVKEGNSDSITTERERKEKNNKKLKETYELNCD |
| | NLSEFYETSNNLRERANSSSLLEDSAFLKKIGLYKVKNNKVNSKVKDEEKRIENIKRKLLKDSSDIMGMY |
| | KAEVVKKLKEKLILIFKHDEEKRIYVTVYDTSKAVPENISKEILVKRNNSKEEYFFEDNNKKYVTEYYTL |
| | EITETNELKVIPAKKLEGKEFKTEKNKENKLMLNNHYCFNVKIIY |
| OWDV01.1 | MENKNKTKPNRGSIVRIIISNYDTKGIKEIKVRYRKQAQLDTFILQTTLDKGNNSILISEFRVKAREKNRYS |
| SEQ ID NO: | FTYDGKEKFSAPSNSVVITKIDNAAPEKFKEIRKYKITLEIDEKCKTGNMITAAIEDLLEDDIAREGIRNPRR |
| 4788 | KASKTERKLIAESICHNYAQIAQCPVEEIDAVKIYKVKRFLSYRSNMLLFFALINDFLCKNLKNKKGEKIN |
| | EIWKMENKGNNKKIDFDENYNILVAQIKEYFTKEIENYNNRIDNIIDKKELLKYSEKKEESEKNKKLEELN |
| | KLKSQKLKILTDEEIKADVIKIIKIFSDLRHSLMHYEYKYFENLFENKKNEELAELLNLNLFKNLTLLRQM |
| | KIENKTNYLEGREEFNIIGKNIKAKEVLGHYNLLAEQKNGFNNFINSFFVQDGTENLEFKKLIDEHFVNAK |
| | KRLERNIKKSKKLEKELEKMEQHYQRLNCAYVWDIHTSTTYKKLYNKRKSLIEEYNKQINEIKDKEVITA |
| | INVELLRIKKEMEEITKSNSLFRLKYKMQIAYAFLEIEFGGNIAKFKDEFDCSKMEEVQKYLKKGVKYLK |
| | YYKDKEAQKNYEFPFEEIFENKDTHNEEWLENTSENNLFKFYILTYLLLPMEFKGDFLGVVKKHYYDIK |
| | NVDFTDESEKELLFLVK |
| OVXT01.1 | MENKNKSNRGSIVRIIISNYDMKGIKELKVRYRKQAQLDTFILQTTLDKSNNSILISEFRVKVREKYRYSFT |
| SEQ ID NO: | YDGKEKFSVPSNSVIVTKIDNAAPEKSKEIRKYKITLGIDEKCKTGSMITAAIEDLLEDDRVREGIRNPRRK |
| 4789 | VSKTERKLIAETICHNYAQIAQCPVEEIDAVKIYKVKRFLSYRSNMLLFFALINDFLCKNLKDKKGEKIREI |
| | WKIENKGNKNWIDYDRYYNILVAQIKEYFTKEIENYNNRIDNIISKKELLKYSEEKKESEKNKKLEELKR |
| | KGREYFKYLDELEILRREKVNTPKREEELIKKIEESSCPGQSFFQAV |
| GCA_ | MEKDKTYKPKQNRSSIIRIILSNYDMIGIKELKILYQKQGGVDTFNLESSIDLDSRKVIIKSFKVKAKEIKRY |
| 002436145.1_ | SFSYDTGDNFSEDKNSVTITKVDNILNKEIRKYKITLSLKEKTTDVILAEVEDKLEESEKKVSGIRTNFRNR |
| ASM243614v1_ | TSKTERKLLSQEVCKNYSEIARVSTEDIDSLKIYKIKRFLSYRSNLLMYFALINNFLCAPLKNEGITEIWKIS |
| genomic | KEDAPLSDERLEKITGHVFNTLSKEIENRVNQLQKRISKNNREIEELKISCNYKNNNKRKYNQLELLNKDL |
| SEQ ID NO: | DKKISELSGYSSKENLKQDLKKVIEIFSNFRHALMHYDYMYFENLFENKACDNLKNLLDLNFFKYTKLIE |
| 4790 | EFKIENKTNYLDGEEKLSVLGKTKNIKNLY |
| OGIA01.1 | MLYSSTFIIESQIEEGVFILKNIKWKAKEKYRYELFIKEVNSTSVEIIKKDRFLNNEIVRGYILNFKVSSKNK |
| SEQ ID NO: | DVVVEIEDILPLKSVQQGEKANIRRITSQTERKLLNEETQISYSKIANCSPKDIDSIKIYKIKRYLSYRSNML |
| 4791 | LFFSLINDFLCEGLYDEKGKKINELWRITNKVDKDIIDERVNKIAKNLDDTLFIELKNYNNGIRKSIEKKNN |
| | SITDCKNKIVSCERKIEKLDEEKNRKKINQFKRDIANCNEKIKEYEESIALKEKEKLFNLGFEKIKADVYKI |
| | LEIYTELRHKLSHYNYIYFENLFENREKDLKLAELLNLNIFNYLTLSKKLRIENKTNYLEENTKFSILGVSG |
| | SAKKYYSLYNTLCEQKNGFNNFINSFFVKDGVENSEFKEKVEAKLKEDIKYLESLETKNNLNKKIPRKNK |
| | ELELLKTQYSELGIVYFWDIHNSLRYKKLYNKRKDNVKEYNQTLKGNRNKTTLRNCGRKLFSKKNEME |
| | KITKRNSIVRLKYKLQIAYAFIMKEYQGDISRFKSDFDISKIEQIKKY |
| OGMZ01.1 | MEGGTKIKANRSSIIRIIISNYDSNGIKEIKVRYNKQAQLDTFLIDSKLENGIFTLKDVKWKAKEKNRYDMI |
| SEQ ID NO: | IGELIDNTVKITKIDKFSNKAIREYIIKFSVSPKNKDVVVVDIKDCMEHNLTIKGERSNTRRDTSQTERKLL |
| 4792 | SKETQISYSKIACCSPENIDSLKIYKIKRYLSYRSNMLLFFSLINDFICEGIKEEKIVELYKITSKVDKNIIEER |
| | VTKIAQYLRENLSNELENYNNGIEKTISKKSNSINDCNNRIESCKKKINKLDKIKNKKQIRNLERIIQDSEN |
| | KIKEYSKIIAEKEKERLVALAEDKIKEDVYKILELYSDLRHKLAHYNYAYFE |
| IMG_ | MNKKQNKSNKNSIIRIIASNYDDKQIKELKVLYTKQGGVDNITIEDMRLDIESERIQFTTAKSPSTQVDIEV |
| 3300010430 | QTEGSMLIQRRQRYTEAVVILRKYKVWGECKKTNDGGTQVKLFVEDLMAEDERNTPINKRRIQSSTERK |
| SEQ ID NO: | LLGSEVKSNYSLILKCTPDEVDSRSIYKAKRFLSYRSNMILFFNFINDFMIKGLPEPEIEKGQIKELWQIVSS |
| 4793 | TKTDPERFNTIIESIAEHIDAHICEFFENHNNYADRMNEKNSEKKGFRPEIIRFDSIDKDSIVEDVKNIVIILS |
| | DFRHKLAHYEFEYFDRLYTGEGVNVTHNKSAIALNKLLNLNIFKELSKITEFKEDKSTTYLDDDDTVRIL |
| | GKSKNAKKFYTMYSKICSRKNGFNQFINSFFTVDGDEDPVFKAAINNEFESRIEFLKTTLKSGKINDKSIK |
| | KRTRTNMEYELKELEQIKTYTGSAYAWDIHLCPEYKTLYNQRKNLIEKQSALISSGNSKVHRKEITEINK |
| | KLLSLKQKMERITKLNSKCRLRYKLQVAYGFLYTEFKMNLKQFGDKFDMSRDELIKGFRSKGEDYLKTR |
| | KNDVEFDLEKLRKKVNDIKQANMDL |
| GCA_ | VEKDKKGEKIDISQEMIEEDLRKILILFSRLRHSMVHYDYEFYQALYSGKDFVISDKNNLENRMISQLLDL |
| 002266425.1_ | NIFKELSKVKLIKDKAISNYLDKNTTIHVLGQDIKAIRLLDIYRDICGSKNGFNKFINTMITISGEEDREYKE |
| ASM226642vl1_ | KVIEHFNKKMENLSTYLEKLEKQDNAKRNNKRVYNLLKQKLIEQQKLKEWFGGPYVYDIHSSKRYKEL |
| genomic | YIERKKLVDRHSKLFEEGLDEKNKKELTKINDELSKLNSEMKEMTKLNSKYRLQYKLQLAFGFILEEFDL |
| SEQ ID NO: | NIDTFINNFDKDKDLIISNFMKKRDIYLNRVLDRGDNRLKNIIKEYKFRDTEDIFCNDRDNNLVKLYILMY |
| 4794 | ILLPVEIRGDFLGFVKKNYYDMKHVDFIDKKDKEDKDTFFHDLRLFEKNIRKLEITDYSLSSGFLSKEHKV |
| | DIEKKINDFINRNGAMKLPEDITIEEFNKSLILPIMKNYQINFKLLNDIEISALFKIAKDRSITFKQAIDEIKNE |
| | DIKKNSKKNDKNNHKDKNINFTQLMKRALHEKIPYKAGMYQIRNNISHIDMEQLYIDPLNSYMNSNKNN |
| | ITISEQIEKIIDVCVTGGVTGKELNNNIINDYYMKKEKLVFNLKLRKQNDIVSIESQEKNKREEFVFKKYGL |
| | DYKDGEINIIEVIQKVNSLQEELRNIKETSKEKLKNKETLFRDISLINGTIRKNINFKIKEMVLDIVRMDEIR |
| | HINIHIYYKGENYTRSNIIKFKYAIDGENKKYYLKQHEINDINLELKDKFVTLICNMDKHPNKNKQTINLE |
| | SNYIQNVKFIIP |
| UOOT01.1 | MDSGNKKKLKPNKSSIVRIIISNFDDKQIKEIKVLYSKQGGVDVIRLNGTEPDEKGRIKFNFKSASNRLEDE |
| SEQ ID NO: | QTYSLGENDGQTFFVTTNEDETELCVTKRSKFTNEIIKEYRLFGEYVATNSNEKKVIVSVSDDIDYSGEKY |
| 4795 | QNSQRKNKRTINQSTNRMLLDLDVINNYRQIGSESDKIDKNVIIDSKEIYKINKFLNYRSDMIIYYQIINNFL |
| | MQGSAKRDDFENEIWKYVKSTDSKTKKKFLNELRVEYLPEDCRKRLKELKTLNFIEEGRNIILAGSELLF |
| | TFLSLRAERKSTIITTNLSFDRWNEIFNDPVLTAALIDRLTHKSYVINMNGDSYRIKETREWLEETN |
| IMG_ | MIVAETPENELDRLKALFELDILDTPLEADFDQLTELAASICGSPIALVSLLDDKRQWFKSHFGLDASETP |
| 3300001201 | RDYAFCAHAINQDEVFEICDSRKDERFHDNPLVTGDPRVIFYAGAPLVTGDGHKLGTVCVIDNEPRSLTD |
| SEQ ID NO: | LQKKQLSILSRQVMALIESRQAVRLKNEAFNKLMSLTKNINEQNKELSQFTTRASHDIQGPIRQIKQLARF |
| 4796 | CQKSAREDSTEFIDDDCEKIISRCDDLSHFISSIFDLTGSSVVVENKREINLKKLVLLAISNNESLIDQYKVN |
| | VTYGVDVSSPFLSEPVRVLQILNNLISNAVKYSNPEKENKTVDVSVSEKNEVIVIKVVDNGLGIPKEFQSR |
| | LFDQFERFHTNSASGTGLGTSIIQKHVKMLLGGITFESDQNGTAFTVTLPFSS |
| mgm4527699.3 | MRKLRAVFYARVSTEEEKQLNALEKQIQENRDIIKEQGWELVGEYIDEGKSGTTTKRRSDYKRLLDDME |
| SEQ ID NO: | GGSFDIVVCKDQDRLQRNTLDWYLFVDNLVRNNLKLYMYLDSKFFTPSEDALITGIKAIIAEEYSRNLSK |
| 4797 | KLNNSNKRRIEKALNGEELSAMGNGKSLGYAIERSEGGKKSKWVQVPEEIEVCKIVWDLYEKYDSIRKV |
| | RDEINNMGYRNSVGKPFTSESIARILKNEKAKGIIVLGKYHHDFDLKKIVRMPEEDLVRVPAPELAYVSEE |
| | RFDRVNARLKAKSNNGRGRNVGRDPLSGKIFCGKCGSVLWRRESSQRNKAGEKKTYYHWACSAKYAK |
| | GDIVCEGTGTTTVAIRNVYKELTSEIEVDRKALRSYFVKWLNQLKTSLSDTSGNAKVEKELEKLERQRA |
| | KLLEAYLEEIISKEDYKSKYADIESKIEEKKKLLAPVEDNEDIKEIERILANLDEELDEFIKTLDVEENKIDF |
| | LIEHTKKITVLENKDLVIELDLVAGAIIAGKKFLLYVHDSMPFPHGRICHEGHREPGRPQRRFFHRLCGYQ |
| | HGGNRERYPLWYPCDFQGEADTLHGA |

In some embodiments, the small Cas proteins are small Cas 13d. Examples of small Cas13d are shown in Table 5 below.

TABLE 5

| Accession | |
|---|---|
| No. | Sequences |
| IMG_ | MKKNSNDKTNAKRMGIKSFIKNGDERFITTSIKNEFPVELKLDVIKKTCEPAHEPVSFDYDPKKIDFE |
| 3300001784 | KPVLKEKLTSGQSGQKLSTRLFIQKDRDICGIRRKYLEKIFNSNFIEEKKDSNLPMQIVAKVLSTEKVF |
| SEQ ID NO: | SNALNKIISQFLSMPRGGVTDNHGEYEIIGNIINHKSLQELNKEKKTKRIKKYLQSVIKNQSYLYNKQF |
| 4798 | LLSLDESKGSRNDIDENELYDYIRFLAILRNGIAHVFYEKNEPETAKESLFRLVDFIKNDKKLEGAFAK |
| | IKIQVNTLYKCRKEEYIKKSGKNFEIIRKIYQNDKPDEKVKDWIRYDFDKSYKYIGLSVAKLGNYTSW |
| | AKDIDNLRDKSNPDSGYAGIMHRLNEFSVYLKVKALSTEEKDKYLKNLISKENCEEKDKYYKNIAQF |
| | FCSSDLKFANVLQMVKEIKKNKGCTSEDKNCKLCVDERKFNDLSVIVYFISCFLDNKDQNIFLSDLIN |
| | RFGALSDLLRIQNKILGAGNKYNENYSFLKNERYVTEIKMELETIFALVKVSYKKEDKAFNRLLEDG |
| | LVMFGFSKDEAGMKVAGLKEIKEKKEGHYKNKSRSFLINSIVNSRKFAYLAKSIDPQKVPAIIKNEHI |
| | VRYILGRINKTNPGQIGRYWRYIMSQNHAGTDKVDDLTNEIIKINIKNILNDAGGWQKSKLNDNNNK |
| | KKLKYQQLIGLYLTVAYIFVKNMLECNARYFSAFAQIEKDYLIYTNSDEFYYIDKNKKNLVTERYLK |
| | LVKDIIEKNKNTVRKDKIFRKKRQRKHLADISKSIIEFEKLPCCIFTLLRNITEHLNVASNIDIIEGYGKR |
| | AGKYHKNAPASYFIFYHYIIQKILADKICTRNLLNIINTYGEPSISFIKIIYVPFAYNLPRYLNLTDARIFC |
| | NMDDK |
| IMG_ | MKKNSNDKTNAKRMGIKSFIKNGDERFITTSIKNEFPVELKLDVIKKTCEPAHEPVSFDYDPKKIDFE |
| 3300001784_2 | KPVLKEKLTSGQSGQKLSTRLFIQKDRDICGIRRKYLEKIFNSNFIEEKKDSNLPMQIVAKVLSTEKVF |
| SEQ ID NO: | SNALNKIISQFLSMPRGGVTDNHGEYEIIGNIINHKSLQELNKEKKTKRIKKYLQSVIKNQSYLYNKQF |
| 4799 | LLSLDESKGSRNDIDENELYDYIRFLAILRNGIAHVFYEKNEPETAKESLFRLVDFIKNDKKLEGAFAK |
| | IKIQVNTLYKCRKEEYIKKSGKNFEIIRKIYQNDKPDEKVKDWIRYDFDKSYKYIGLSVAKLGNYTSW |
| | AKDIDNLRDKSNPDSGYAGIMHRLNEFSVYLKVKALSTEEKDKYLKNLISKENCEEKDKYYKNIAQF |
| | FCSSDLKFANVLQMVKEIKKNKGCTSEDKNCKLCVDERKFNDLSVIVYFISCFLDNKDQNIFLSDLIN |
| | RFGALSDLLRIQNKILGAGNKYNENYSFLKNERYVTEIKMELETIFALVKVSYKKEDKAFNRLLEDG |
| | LVMFGFSKDEAGMKVAGLKEIKEKKEGHYKNKSRSFLINSIVNSRKFAYLAKSIDPQKVPAIIKNEHI |
| | VRYILGRINKTNPGQIGRYWRYIMSQNHAGTDKVDDLTNEIIKINIKNILNDAGGWQKSKLNDNNNK |
| | KKLKYQQLIGLYLTVAYIFVKNMLECNARYFSAFAQIEKDYLIYTNSDEFYYIDKNKKNLVTERYLK |
| | LVKDIIEKNKNTVRKDKIFRKKRQRKHLADISKSIIEFEKLPCCIFTLLRNITEHLNVASNIDIIEGYGKR |
| | AGKYHKNAPASYFIFYHYIIQKILADKICTRNLLNIINTYGEPSISFIKIIYVPFAYNLPRYLNLTDARIFC |
| | NMDDK |
| IMG_ | MKKNSNDKTNAKRMGIKSFIKNGDERFITTSIKNEFPVELKLDVIKKTCEPAHEPVSFDYDPKKIDFE |
| 3300001784_3 | KPVLKEKLTSGQSGQKLSTRLFIQKDRDICGIRRKYLEKIFNSNFIEEKKDSNLPMQIVAKVLSTEKVF |
| SEQ ID NO: | SNALNKIISQFLSMPRGGVTDNHGEYEIIGNIINHKSLQELNKEKKTKRIKKYLQSVIKNQSYLYNKQF |
| 4800 | LLSLDESKGSRNDIDENELYDYIRFLAILRNGIAHVFYEKNEPETAKESLFRLVDFIKNDKKLEGAFAK |
| | nCIQVNTLYKCRKEEYIKKSGKNFEIIRKIYQNDKPDEKVKDWIRYDFDKSYKYIGLSVAKLGNYTSW |
| | AKDIDNLRDKSNPDSGYAGIMHRLNEFSVYLKVKALSTEEKDKYLKNLISKENCEEKDKYYKNIAQF |
| | FCSSDLKFANVLQMVKEIKKNKGCTSEDKNCKLCVDERKFNDLSVIVYFISCFLDNKDQNIFLSDLIN |
| | RFGALSDLLRIQNKILGAGNKYNENYSFLKNERYVTEIKMELETIFALVKVSYKKEDKAFNRLLEDG |
| | LVMFGFSKDEAGMKVAGLKEIKEKKEGHYKNKSRSFLINSIVNSRKFAYLAKSIDPQKVPAIIKNEHI |
| | VRYILGRINKTNPGQIGRYWRYIMSQNHAGTDKVDDLTNEIIKINIKNILNDAGGWQKSKLNDNNNK |
| | KKLKYQQLIGLYLTVAYIFVKNMLECNARYFSAFAQIEKDYLIYTNSDEFYYIDKNKKNLVTERYLK |
| | LVKDIIEKNKNTVRKDKIFRKKRQRKHLADISKSIIEFEKLPCCIFTLLRNITEHLNVASNIDIIEGYGKR |
| | AGKYHKNAPASYFIFYHYIIQKILADKICTRNLLNIINTYGEPSISFIKIIYVPFAYNLPRYLNLTDARIFC |
| | NMDDK |
| IMG_ | MKKNSNDKTNAKRMGIKSFIKNGDERFITTSIKNEFPVELKLDVIKKTCEPAHEPVSFDYDPKKIDFE |
| 3300001784_4 | KPVLKEKLTSGQSGQKLSTRLFIQKDRDICGIRRKYLEKIFNSNFIEEKKDSNLPMQIVAKVLSTEKVF |
| SEQ ID NO: | SNALNKIISQFLSMPRGGVTDNHGEYEIIGNIINHKSLQELNKEKKTKRIKKYLQSVIKNQSYLYNKQF |
| 4801 | LLSLDESKGSRNDIDENELYDYIRFLAILRNGIAHVFYEKNEPETAKESLFRLVDFIKNDKKLEGAFAK |
| | IKIQVNTLYKCRKEEYIKKSGKNFEIIRKIYQNDKPDEKVKDWIRYDFDKSYKYIGLSVAKLGNYTSW |
| | AKDIDNLRDKSNPDSGYAGIMHRLNEFSVYLKVKALSTEEKDKYLKNLISKENCEEKDKYYKNIAQF |
| | FCSSDLKFANVLQMVKEIKKNKGCTSEDKNCKLCVDERKFNDLSVIVYFISCFLDNKDQNIFLSDLIN |
| | RFGALSDLLRIQNKILGAGNKYNENYSFLKNERYVTEIKMELETIFALVKVSYKKEDKAFNRLLEDG |
| | LVMFGFSKDEAGMKVAGLKEIKEKKEGHYKNKSRSFLINSIVNSRKFAYLAKSIDPQKVPAIIKNEHI |
| | VRYILGRINKTNPGQIGRYWRYIMSQNHAGTDKVDDLTNEIIKINIKNILNDAGGWQKSKLNDNNNK |
| | KKLKYQQLIGLYLTVAYIFVKNMLECNARYFSAFAQIEKDYLIYTNSDEFYYIDKNKKNLVTERYLK |
| | LVKDIIEKNKNTVRKDKIFRKKRQRKHLADISKSIIEFEKLPCCIFTLLRNITEHLNVASNIDIIEGYGKR |
| | AGKYHKNAPASYFIFYHYIIQKILADKICTRNLLNIINTYGEPSISFIKIIYVPFAYNLPRYLNLTDARIFC |
| | NMDDK |
| IMG_ | MKKNSNDKTNAKRMGIKSFIKNGDERFITTSIKNEFPVELKLDVIKKTCEPAHEPVSFDYDPKKIDFE |
| 3300028582 | KPVLKEKLTSGQSGQKLSTRLFIQKDRDICGIRRKYLEKIFNSNFIEEKKDSNLPMQIVAKVLSTEKVF |
| SEQ ID NO: | SNALNKIISQFLSMPRGGVTDNHGEYEIIGNIINHKSLQELNKEKKTKRIKKYLQSVIKNQSYLYNKQF |
| 4802 | LLSLDESKGSRNDIDENELYDYIRFLAILRNGIAHVFYEKNEPETAKESLFRLVDFIKNDKKLEGAFAK |
| | IKIQVNTLYKCRKEEYIKKSGKNFEIIRKIYQNDKPDEKVKDWIRYDFDKSYKYIGLSVAKLGNYTSW |
| | AKDIDNLRDKSNPDSGYAGIMHRLNEFSVYLKVKALSTEEKDKYLKNLISKENCEEKDKYYKNIAQF |
| | FCSSDLKFANVLQMVKEIKKNKGCTSEDKNCKLCVDERKFNDLSVIVYFISCFLDNKDQNIFLSDLIN |
| | RFGALSDLLRIQNKILGAGNKYNENYSFLKNERYVTEIKMELETIFALVKVSYKKEDKAFNRLLEDG |
| | LVMFGFSKDEAGMKVAGLKEIKEKKEGHYKNKSRSFLINSIVNSRKFAYLAKSIDPQKVPAIIKNEHI |
| | VRYILGRINKTNPGQIGRYWRYIMSQNHAGTDKVDDLTNEIIKINIKNILNDAGGWQKSKLNDNNNK |
| | KKLKYQQLIGLYLTVAYIFVKNMLECNARYFSAFAQIEKDYLIYTNSDEFYYIDKNKKNLVTERYLK |
| | LVKDIIEKNKNTVRKDKIFRKKRQRKHLADISKSIIEFEKLPCCIFTLLRNITEHLNVASNIDIIEGYGKR |
| | AGKYHKNAPASYFIFYHYIIQKILADKICTRNLLNIINTYGEPSISFIKIIYVPFAYNLPRYLNLTDARIFC |
| | NMDDK |
| IMG_ | MKKNSNDKTNAKRMGIKSFIKNGDERFITTSIKNEFPVELKLDVIKKTCEPAHEPVSFDYDPKKIDFE |
| 3300028326 | KPVLKEKLTSGQSGQKLSTRLFIQKDRDICGIRRKYLEKIFNSNFIEEKKDSNLPMQIVAKVLSTEKVF |
| SEQ ID NO: | SNALNKIISQFLSMPRGGVTDNHGEYEIIGNIINHKSLQELNKEKKTKRIKKYLQSVIKNQSYLYNKQF |
| 4803 | LLSLDESKGSRNDIDENELYDYIRFLAILRNGIAHVFYEKNEPETAKESLFRLVDFIKNDKKLEGAFAK |
| | nCIQVNTLYKCRKEEYIKKSGKNFEIIRKIYQNDKPDEKVKDWIRYDFDKSYKYIGLSVAKLGNYTSW |
| | AKDIDNLRDKSNPDSGYAGIMHRLNEFSVYLKVKALSTEEKDKYLKNLISKENCEEKDKYYKNIAQF |
| | FCSSDLKFANVLQMVKEIKKNKGCTSEDKNCKLCVDERKFNDLSVIVYFISCFLDNKDQNIFLSDLIN |
| | RFGALSDLLRIQNKILGAGNKYNENYSFLKNERYVTEIKMELETIFALVKVSYKKEDKAFNRLLEDG |
| | LVMFGFSKDEAGMKVAGLKEIKEKKEGHYKNKSRSFLINSIVNSRKFAYLAKSIDPQKVPAIIKNEHI |
| | VRYILGRINKTNPGQIGRYWRYIMSQNHAGTDKVDDLTNEIIKINIKNILNDAGGWQKSKLNDNNNK |
| | KKLKYQQLIGLYLTVAYIFVKNMLECNARYFSAFAQIEKDYLIYTNSDEFYYIDKNKKNLVTERYLK |
| | LVKDIIEKNKNTVRKDKIFRKKRQRKHLADISKSIIEFEKLPCCIFTLLRNITEHLNVASNIDIIEGYGKR |
| | AGKYHKNAPASYFIFYHYIIQKILADKICTRNLLNIINTYGEPSISFIKIIYVPFAYNLPRYLNLTDARIFC |
| | NMDDK |
| IMG_ | MKKNSNDKTNAKRMGIKSFIKNGDERFITTSIKNEFPVELKLDVIKKTCEPAHEPVSFDYDPKKIDFE |
| 3300016738 | KPVLKEKLTSGQSGQKLSTRLFIQKDRDICGIRRKYLEKIFNSNFIEEKKDSNLPMQIVAKVLSTEKVF |
| SEQ ID NO: | SNALNKIISQFLSMPRGGVTDNHGEYEIIGNIINHKSLQELNKEKKTKRIKKYLQSVIKNQSYLYNKQF |
| 4804 | LLSLDESKGSRNDIDENELYDYIRFLAILRNGIAHVFYEKNEPETAKESLFRLVDFIKNDKKLEGAFAK |
| | IKIQVNTLYKCRKEEYIKKSGKNFEIIRKIYQNDKPDEKVKDWIRYDFDKSYKYIGLSVAKLGNYTSW |
| | AKDIDNLRDKSNPDSGYAGIMHRLNEFSVYLKVKALSTEEKDKYLKNLISKENCEEKDKYYKNIAQF |
| | FCSSDLKFANVLQMVKEIKKNKGCTSEDKNCKLCVDERKFNDLSVIVYFISCFLDNKDQNIFLSDLIN |
| | RFGALSDLLRIQNKILGAGNKYNENYSFLKNERYVTEIKMELETIFALVKVSYKKEDKAFNRLLEDG |
| | LVMFGFSKDEAGMKVAGLKEIKEKKEGHYKNKSRSFLINSIVNSRKFAYLAKSIDPQKVPAIIKNEHI |
| | VRYILGRINKTNPGQIGRYWRYIMSQNHAGTDKVDDLTNEIIKINIKNILNDAGGWQKSKLNDNNNK |
| | KKLKYQQLIGLYLTVAYIFVKNMLECNARYFSAFAQIEKDYLIYTNSDEFYYIDKNKKNLVTERYLK |
| | LVKDIIEKNKNTVRKDKIFRKKRQRKHLADISKSIIEFEKLPCCIFTLLRNITEHLNVASNIDIIEGYGKR |
| | AGKYHKNAPASYFIFYHYIIQKILADKICTRNLLNIINTYGEPSISFIKIIYVPFAYNLPRYLNLTDARIFC |
| | NMDDK |
| IMG_ | MKKNSNDKTNAKRMGIKSFIKNGDERFITTSIKNEFPVELKLDVIKKTCEPAHEPVSFDYDPKKIDFE |
| 3300004628 | KPVLKEKLTSGQSGQKLSTRLFIQKDRDICGIRRKYLEKIFNSNFIEEKKDSNLPMQIVAKVLSTEKVF |
| SEQ ID NO: | SNALNKIISQFLSMPRGGVTDNHGEYEIIGNIINHKSLQELNKEKKTKRIKKYLQSVIKNQSYLYNKQF |
| 4805 | LLSLDESKGSRNDIDENELYDYIRFLAILRNGIAHVFYEKNEPETAKESLFRLVDFIKNDKKLEGAFAK |
| | IKIQVNTLYKCRKEEYIKKSGKNFEIIRKIYQNDKPDEKVKDWIRYDFDKSYKYIGLSVAKLGNYTSW |
| | AKDIDNLRDKSNPDSGYAGIMHRLNEFSVYLKVKALSTEEKDKYLKNLISKENCEEKDKYYKNIAQF |
| | FCSSDLKFANVLQMVKEIKKNKGCTSEDKNCKLCVDERKFNDLSVIVYFISCFLDNKDQNIFLSDLIN |
| | RFGALSDLLRIQNKILGAGNKYNENYSFLKNERYVTEIKMELETIFALVKVSYKKEDKAFNRLLEDG |
| | LVMFGFSKDEAGMKVAGLKEIKEKKEGHYKNKSRSFLINSIVNSRKFAYLAKSIDPQKVPAIIKNEHI |
| | VRYILGRINKTNPGQIGRYWRYIMSQNHAGTDKVDDLTNEIIKINIKNILNDAGGWQKSKLNDNNNK |
| | KKLKYQQLIGLYLTVAYIFVKNMLECNARYFSAFAQIEKDYLIYTNSDEFYYIDKNKKNLVTERYLK |
| | LVKDIIEKNKNTVRKDKIFRKKRQRKHLADISKSIIEFEKLPCCIFTLLRNITEHLNVASNIDIIEGYGKR |
| | AGKYHKNAPASYFIFYHYIIQKILADKICTRNLLNIINTYGEPSISFIKIIYVPFAYNLPRYLNLTDARIFC |
| | NMDDK |
| IMG_330002 | MKKNSNDKTNAKRMGIKSFIKNGDERFITTSIKNEFPVELKLDVIKKTCEPAHEPVSFDYDPKKIDFE |
| 8580 | KPVLKEKLTSGQSGQKLSTRLFIQKDRDICGIRRKYLEKIFNSNFIEEKKDSNLPMQIVAKVLSTEKVF |
| SEQ ID NO: | SNALNKIISQFLSMPRGGVTDNHGEYEIIGNIINHKSLQELNKEKKTKRIKKYLQSVIKNQSYLYNKQF |
| 4806 | LLSLDESKGSRNDIDENELYDYIRFLAILRNGIAHVFYEKNEPETAKESLFRLVDFIKNDKKLEGAFAK |
| | IKIQVNTLYKCRKEEYIKKSGKNFEIIRKIYQNDKPDEKVKDWIRYDFDKSYKYIGLSVAKLGNYTSW |
| | AKDIDNLRDKSNPDSGYAGIMHRLNEFSVYLKVKALSTEEKDKYLKNLISKENCEEKDKYYKNIAQF |
| | FCSSDLKFANVLQMVKEIKKNKGCTSEDKNCKLCVDERKFNDLSVIVYFISCFLDNKDQNIFLSDLIN |
| | RFGALSDLLRIQNKILGAGNKYNENYSFLKNERYVTEIKMELETIFALVKVSYKKEDKAFNRLLEDG |
| | LVMFGFSKDEAGMKVAGLKEIKEKKEGHYKNKSRSFLINSIVNSRKFAYLAKSIDPQKVPAIIKNEHI |
| | VRYILGRINKTNPGQIGRYWRYIMSQNHAGTDKVDDLTNEIIKINIKNILNDAGGWQKSKLNDNNNK |
| | KKLKYQQLIGLYLTVAYIFVKNMLECNARYFSAFAQIEKDYLIYTNSDEFYYIDKNKKNLVTERYLK |
| | LVKDIIEKNKNTVRKDKIFRKKRQRKHLADISKSIIEFEKLPCCIFTLLRNITEHLNVASNIDIIEGYGKR |
| | AGKYHKNAPASYFIFYHYIIQKILADKICTRNLLNIINTYGEPSISFIKIIYVPFAYNLPRYLNLTDARIFC |
| | NMDDK |
| IMG_ | MKKNSNDKTNAKRMGIKSFIKNGDERFITTSIKNEFPVELKLDVIKKTCEPAHEPVSFDYDPKKIDFE |
| 3300012889 | KPVLKEKLTSGQSGQKLSTRLFIQKDRDICGIRRKYLEKIFNSNFIEEKKDSNLPMQIVAKVLSTEKVF |
| SEQ ID NO: | SNALNKIISQFLSMPRGGVTDNHGEYEIIGNIINHKSLQELNKEKKTKRIKKYLQSVIKNQSYLYNKQF |
| 4807 | LLSLDESKGSRNDIDENELYDYIRFLAILRNGIAHVFYEKNEPETAKESLFRLVDFIKNDKKLEGAFAK |
| | IKIQVNTLYKCRKEEYIKKSGKNFEIIRKIYQNDKPDEKVKDWIRYDFDKSYKYIGLSVAKLGNYTSW |
| | AKDIDNLRDKSNPDSGYAGIMHRLNEFSVYLKVKALSTEEKDKYLKNLISKENCEEKDKYYKNIAQF |
| | FCSSDLKFANVLQMVKEIKKNKGCTSEDKNCKLCVDERKFNDLSVIVYFISCFLDNKDQNIFLSDLIN |
| | RFGALSDLLRIQNKILGAGNKYNENYSFLKNERYVTEIKMELETIFALVKVSYKKEDKAFNRLLEDG |
| | LVMFGFSKDEAGMKVAGLKEIKEKKEGHYKNKSRSFLINSIVNSRKFAYLAKSIDPQKVPAIIKNEHI |
| | VRYILGRINKTNPGQIGRYWRYIMSQNHAGTDKVDDLTNEIIKINIKNILNDAGGWQKSKLNDNNNK |
| | KKLKYQQLIGLYLTVAYIFVKNMLECNARYFSAFAQIEKDYLIYTNSDEFYYIDKNKKNLVTERYLK |
| | LVKDIIEKNKNTVRKDKIFRKKRQRKHLADISKSIIEFEKLPCCIFTLLRNITEHLNVASNIDIIEGYGKR |
| | AGKYHKNAPASYFIFYHYIIQKILADKICTRNLLNIINTYGEPSISFIKIIYVPFAYNLPRYLNLTDARIFC |
| | NMDDK |
| IMG_ | MKKNSNDKTNAKRMGIKSFIKNGDERFITTSIKNEFPVELKLDVIKKTCEPAHEPVSFDYDPKKIDFE |
| 3300012886 | KPVLKEKLTSGQSGQKLSTRLFIQKDRDICGIRRKYLEKIFNSNFIEEKKDSNLPMQIVAKVLSTEKVF |
| SEQ ID NO: | SNALNKIISQFLSMPRGGVTDNHGEYEIIGNIINHKSLQELNKEKKTKRIKKYLQSVIKNQSYLYNKQF |
| 4808 | LLSLDESKGSRNDIDENELYDYIRFLAILRNGIAHVFYEKNEPETAKESLFRLVDFIKNDKKLEGAFAK |
| | IKIQVNTLYKCRKEEYIKKSGKNFEIIRKIYQNDKPDEKVKDWIRYDFDKSYKYIGLSVAKLGNYTSW |
| | AKDIDNLRDKSNPDSGYAGIMHRLNEFSVYLKVKALSTEEKDKYLKNLISKENCEEKDKYYKNIAQF |
| | FCSSDLKFANVLQMVKEIKKNKGCTSEDKNCKLCVDERKFNDLSVIVYFISCFLDNKDQNIFLSDLIN |
| | RFGALSDLLRIQNKILGAGNKYNENYSFLKNERYVTEIKMELETIFALVKVSYKKEDKAFNRLLEDG |
| | LVMFGFSKDEAGMKVAGLKEIKEKKEGHYKNKSRSFLINSIVNSRKFAYLAKSIDPQKVPAIIKNEHI |
| | VRYILGRINKTNPGQIGRYWRYIMSQNHAGTDKVDDLTNEIIKINIKNILNDAGGWQKSKLNDNNNK |
| | KKLKYQQLIGLYLTVAYIFVKNMLECNARYFSAFAQIEKDYLIYTNSDEFYYIDKNKKNLVTERYLK |
| | LVKDIIEKNKNTVRKDKIFRKKRQRKHLADISKSIIEFEKLPCCIFTLLRNITEHLNVASNIDIIEGYGKR |
| | AGKYHKNAPASYFIFYHYIIQKILADKICTRNLLNIINTYGEPSISFIKIIYVPFAYNLPRYLNLTDARIFC |
| | NMDDK |
| UZMO01.1 | MAKKNKMKPRELREAQKKARQLKAAEINNNATPTIAAMPAAEVIAPAAEKKKSSVKAAGMKSILV |
| SEQ ID NO: | SKNKMYITSFGKGNSAVLEYEVDNNDYNQTQLSSKNSSNIELRGVNEVNITFSSKHGFESGVEINTSN |
| 4809 | PTHRSGESSSVRGDMLGLKSELEKRFFGKTFDDNIHIQLIYNILDIEKILAVYVTNIVYALNNMLGIKD |
| | SESYDDFIGYLSARNTYKVFTHPDKSNLSDKVKGNIKKSFSTFNDLLKTKRLGYFGLEEPKTKDTRVS |
| | QAYKKRVYHMLAIVGQIRQSVFHDKSSKLDEDLYSFIDIIDPEYRETLDYLVDERFDSINKGFIQGNK |
| | VNISLLIDMMKGYEADDIIRLYYDFIVLKSQKNLGFSIKKLREKMLDEYGFRFKDKQYDSVRSKMYK |
| | LMDFLLFCNYYRNDVVAGEALVRKLRFSMTDDEKRGDIC |
| UPPC01.1_2 | MAKKNKMKPRELREAQKKARQLKAAEINNNAAPAIAAMPAAEVIAPAAEKKKSSVKAAGMKSILV |
| SEQ ID NO: | SKNKMYITSFGKGNSAVLEYEVDKVDNNNYNKTQLSSESSSNIELCGVTKVNITFSSKHGLESGVEIS |
| 4810 | TSNPTHRSGESSPVRWDMLGLKSELEKRFFGKTFDDNIHIQLIYNILDIEKILAVYVTNIVYALNNMLG |
| | VKGSESHDDFIGYLSTNNTYDVFIDPDNSSLSDDKKANVRKSLSKFNVLLKTKRLGYFGLEEPKTKD |
| | TRASQAYKKRVYHMLAIVGQIRQCVFHDKSGAKRFDLYSFINNIYPEYRDTLDYLVEERLKSINKDFI |
| | EGNKVNISLLIDMMKGYEADDIIRLYYDFIVLKSQKNLGFSIKKLREKMLDEYGFRFKDKQYDSVRS |
| | KMYKLMDFLLFCNYYRNDVAAGEALVRKLRFSMTDDEKEGIYADEAAKLWGKFRNDFENIADHM |
| | NGDVIKELGKADMDFDEKILDSEKKNASDLLYFSKMIYMLTYFLDGKEINDLLTTLISKFDNIKEF |
| OWCF01.1 | MAKKNKMKPRELREAQKKARQLKAAEINNNAAPAIAAMPAAEVIAPAAEKKKSSVKAAGMKSILV |
| SEQ ID NO: | SKNKMYITSFGKGNSAVLEYEVDKVDNNNYNKTQLSSESSSNIELCGVTKVNITFSSKHGLESGVEIS |
| 4811 | TSNPTHRSGESSPVRWDMLGLKSELEKRFFGKTFDDNIHIQLIYNILDIEKILAVYVTNIVYALNNMLG |
| | VKGSESHDDFIGYLSTNNTYDVFIDPDNSSLSDDKKANVRKSLSKFNVLLKTKRLGYFGLEEPKTKD |
| | TRASQAYKKRVYHMLAIVGQIRQCVFHDKSGAKRFDLYSFINNIYPEYRDTLDYLVEERLKSINKDFI |
| | EGNKVNISLLIDMMKGYEADDIIRLYYDFIVLKSQKNLGFSIKKLREKMLDEYGFRFKDKQYDSVRS |
| | KMYKLMDFLLFCNYYRNDVAAGEALVRKLRFSMTDDEKEGIYADEAAKLWGKFRNDFENIADHM |
| | NGDVIKELGKADMDFDEKILDSEKKNASDLLYFSKMIYMLTYFLDGKEINDLLTTLISKFDNIKEF |
| OGLN01.1 | MAKKNKMKPRELREAQKKARQLKAAEINNNAAPAIAAMPAAEVIAPAAEKKKSSVKAAGMKSILV |
| SEQ ID NO: | SKNKMYITSFGKGNSAVLEYEVDKVDNNNYNKTQLSSESSSNIELCGVTKVNITFSSKHGLESGVEIS |
| 4812 | TSNPTHRSGESSPVRWDMLGLKSELEKRFFGKTFDDNIHIQLIYNILDIEKILAVYVTNIVYALNNMLG |
| | VKGSESHDDFIGYLSTNNTYDVFIDPDNSSLSDDKKANVRKSLSKFNVLLKTKRLGYFGLEEPKTKD |
| | TRASQAYKKRVYHMLAIVGQIRQCVFHDKSGAKRFDLYSFINNIYPEYRDTLDYLVEERLKSINKDFI |
| | EGNKVNISLLIDMMKGYEADDIIRLYYDFIVLKSQKNLGFSIKKLREKMLDEYGFRFKDKQYDSVRS |
| | KMYKLMDFLLFCNYYRNDVAAGEALVRKLRFSMTDDEKEGIYADEAAKLWGKFRNDFENIADHM |
| | NGDVIKELGKADMDFDEKILDSEKKNASDLLYFSKMIYMLTYFLDGKEINDLLTTLISKFDNIKEF |
| UZLM01.1 | MAKKNKMKPRELREAQKKARQLKAAEINNNAAPAIAAMPAAEVIAPAAEKKKSSVKAAGMKSILV |
| SEQ ID NO: | SKNKMYITSFGKGNSAVLEYEVDKVDNNNYNKTQLSSESSSNIELCGVTKVNITFSSKHGLESGVEIS |
| 4813 | TSNPTHRSGESSPVRWDMLGLKSELEKRFFGKTFDDNIHIQLIYNILDIEKILAVYVTNIVYALNNMLG |
| | VKGSESHDDFIGYLSTNNTYDVFIDPDNSSLSDDKKANVRKSLSKFNVLLKTKRLGYFGLEEPKTKD |
| | TRASQAYKKRVYHMLAIVGQIRQCVFHDKSGAKRFDLYSFINNIYPEYRDTLDYLVEERLKSINKDFI |
| | EGNKVNISLLIDMMKGYEADDIIRLYYDFIVLKSQKNLGFSIKKLREKMLDEYGFRFKDKQYDSVRS |
| | KMYKLMDFLLFCNYYRNDVAAGEALVRKLRFSMTDDEKEGIYADEAAKLWGKFRNDFENIADHM |
| | NGDVIKELGKADMDFDEKILDSEKKNASDLLYFSKMIYMLTYFLDGKEINDLLTTLISKFDNIKEF |
| OGWR01.1_2 | MAKKNKMKPRELREAQKKARQLKAAEINNNAAPAIAAMPAAEVIAPAAEKKKSSVKAAGMKSILV |
| SEQ ID NO: | SKNKMYITSFGKGNSAVLEYEVDKVDNNNYNKTQLSSESSSNIELCGVTKVNITFSSKHGLESGVEIS |
| 4814 | TSNPTHRSGESSPVRWDMLGLKSELEKRFFGKTFDDNIHIQLIYNILDIEKILAVYVTNIVYALNNMLG |
| | VKGSESHDDFIGYLSTNNTYDVFIDPDNSSLSDDKKANVRKSLSKFNVLLKTKRLGYFGLEEPKTKD |
| | TRASQAYKKRVYHMLAIVGQIRQCVFHDKSGAKRFDLYSFINNIYPEYRDTLDYLVEERLKSINKDFI |
| | EGNKVNISLLIDMMKGYEADDIIRLYYDFIVLKSQKNLGFSIKKLREKMLDEYGFRFKDKQYDSVRS |
| | KMYKLMDFLLFCNYYRNDVAAGEALVRKLRFSMTDDEKEGIYADEAAKLWGKFRNDFENIADHM |
| | NGDVIKELGKADMDFDEKILDSEKKNASDLLYFSKMIYMLTYFLDGKEINDLLTTLISKFDNIKEF |
| OHAD01.1 | MAKKNKMKPRELREAQKKARQLKAAEINNNAAPAIAAMPAAEVIAPAAEKKKSSVKAAGMKSILV |
| SEQ ID NO: | SKNKMYITSFGKGNSAVLEYEVDKVDNNNYNKTQLSSESSSNIELCGVTKVNITFSSKHGLESGVEIS |
| 4815 | TSNPTHRSGESSPVRWDMLGLKSELEKRFFGKTFDDNIHIQLIYNILDIEKILAVYVTNIVYALNNMLG |
| | VKGSESHDDFIGYLSTNNTYDVFIDPDNSSLSDDKKANVRKSLSKFNVLLKTKRLGYFGLEEPKTKD |
| | TRASQAYKKRVYHMLAIVGQIRQCVFHDKSGAKRFDLYSFINNIYPEYRDTLDYLVEERLKSINKDFI |
| | EGNKVNISLLIDMMKGYEADDIIRLYYDFIVLKSQKNLGFSIKKLREKMLDEYGFRFKDKQYDSVRS |
| | KMYKLMDFLLFCNYYRNDVAAGEALVRKLRFSMTDDEKEGIYADEAAKLWGKFRNDFENIADHM |
| | NGDVIKELGKADMDFDEKILDSEKKNASDLLYFSKMIYMLTYFLDGKEINDLLTTLISKFDNIKEF |
| USXY01.1_2 | MAKKNKMKPRELREAQKKARQLKAAEINNNAAPAIAAMPAAEVIAPAAEKKKSSVKAAGMKSILV |
| SEQ ID NO: | SKNKMYITSFGKGNSAVLEYEVDKVDNNNYNKTQLSSESSSNIELCGVTKVNITFSSKHGLESGVEIS |
| 4816 | TSNPTHRSGESSPVRWDMLGLKSELEKRFFGKTFDDNIHIQLIYNILDIEKILAVYVTNIVYALNNMLG |
| | VKGSESHDDFIGYLSTNNTYDVFIDPDNSSLSDDKKANVRKSLSKFNVLLKTKRLGYFGLEEPKTKD |
| | TRASQAYKKRVYHMLAIVGQIRQCVFHDKSGAKRFDLYSFINNIYPEYRDTLDYLVEERLKSINKDFI |
| | EGNKVNISLLIDMMKGYEADDIIRLYYDFIVLKSQKNLGFSIKKLREKMLDEYGFRFKDKQYDSVRS |
| | KMYKLMDFLLFCNYYRNDVAAGEALVRKLRFSMTDDEKEGIYADEAAKLWGKFRNDFENIADHM |
| | NGDVIKELGKADMDFDEKILDSEKKNASDLLYFSKMIYMLTYFLDGKEINDLLTTLISKFDNIKEF |
| OZEI01.1 | MAKKNKMKPRELREAQKKARQFKAAEINNNAAPAIAAMPAAEVIAPVAEKKKSSVKAAGMKSILV |
| SEQ ID NO: | SENKMYITSFGKGNSAVLEYEVDNNDYNKTQLSSKDNSNIELGDVDEVNITFSSKHGFGSGVEINTS |
| 4817 | NPTHRSGESSPVRWDMLGLKSELEKRFFGKTFDDNIHIQLIYNILDIEKILAVYVTNIVYALNNMLGV |
| | KGSESHDDFIGYLSTNNIYDVFIDPDNSSLSDDKKANVRKSLSKFNVLLKTKRLGYFGLEEPKTKDNR |
| | VSEAYKKRVYHMLAIVGQIRQCVFHDKSGAKRFDLYSFINNIDPEYRDTLDYLVDERFDSINKGFVQ |
| | GNKVNISLLIDMMKGYEADDIIRLYYDFIVLKSQKNLGFSIKKLREKMLDEYGYRFKDKQYDSVRSK |
| | MYKLMDFLLFCNYYRNDVVAGEALVRKLRFSMTDDEKEGIYADEAEKLWGKFRNDFENIADHMN |
| | GDVIKELGKADMDFDEKILDSEKKNASDLLYFSKMIYMLTYFLDGKEINDLLTTLISKFDNIKEFLKI |
| | MKSSAVDVECELTAGYKLFNDSQRITNELFIVKNIASMRKPAA |
| OCPU01.1 | MAKKNKMKPRELREAQKKARQFKAAEINNNAVPAIAAMPAAEAAAPAAEKKKSSVKAAGMKSIL |
| SEQ ID NO: | VSENKMYITSFGKGNSAVLEYEVDNNDYNKTQLSSEDSSNIELCGVNEVNITFSSKHGFESGVEINTS |
| 4818 | NPTHRSGESSPVRGDMLGLKSELEKRFFGKTFDDNIHIQLIYNILDIEKILAVYVTNIVYALNNMLGV |
| | KGSESHDDFIGYLSTNNIYDVFIDPDNSSLSDDKKANVRKSLSKFNALLKTKRLGYFGLEEPKTKDTR |
| | ASEAYKKRVYHMLAIVGQIRQCVFHDKSGAKRFDLYSFINNIDPEYRDTLDYLVDERFDSINKGFIQG |
| | NKVNISLLIDMMKGYEADDIIRLYYDFIVLKSQKNLGFSIKKLREKMLEEYGYRFKDKQYDSVRSKM |
| | YKLMDFLLFCNYYRNDVVAGEALVRKLRFSMTDDEKEGIYADEAAKLWGKFRNDFENIADHMNG |
| | DVIKELGKADMDFDEKILDSEKKNASDLLYFSKMIYMLTYFLDGKEINDLLTTLISKFDNIKEFLKIM |
| | KSSAVDVECELTAGYKLFNDSQRITNELFIVKNIASMRKPAASAKLTMFRDALTILGIDDKITDDRISE |
| | ILKLKEKGKGIHGLRNFITNNVTESSRFVYLIKYANAQKIREVAKNEKWMFVLGGIPDTQIERYYKS |
| | CVEFPDMNSSLGVKRSELARMIKNISFDDFKNVKQQAKGRENVAKERAKAVIGLYLTVMYLLVKNL |
| | VNVNARYVIAIHCLERDFGLYKEIIPELASKNLKNDYRILSQTLCELCDKSPNLFCASALKSILIMQTA |
| | A |
| OGTB01.1 | MAKKNKMKPRELREAQKKARQFKAAEINNNAVPAIAAMPAAEAAAPAAEKKKSSVKAAGMKSIL |
| SEQ ID NO: | VSENKMYITSFGKGNSAVLEYEVDNNDYNKTQLSSEDSSNIELCGVNEVNITFSSKHGFESGVEINTS |
| 4819 | NPTHRSGESSPVRGDMLGLKSELEKRFFGKTFDDNIHIQLIYNILDIEKILAVYVTNIVYALNNMLGV |
| | KGSESHDDFIGYLSTNNIYDVFIDPDNSSLSDDKKANVRKSLSKFNALLKTKRLGYFGLEEPKTKDTR |
| | ASEAYKKRVYHMLAIVGQIRQCVFHDKSGAKRFDLYSFINNIDPEYRDTLDYLVDERFDSINKGFIQG |
| | NKVNISLLIDMMKGYEADDIIRLYYDFIVLKSQKNLGFSIKKLREKMLEEYGYRFKDKQYDSVRSKM |
| | YKLMDFLLFCNYYRNDVVAGEALVRKLRFSMTDDEKEGIYADEAAKLWGKFRNDFENIADHMNG |
| | DVIKELGKADMDFDEKILDSEKKNASDLLYFSKMIYMLTYFLDGKEINDLLTTLISKFDNIKEFLKIM |
| | KSSAVNVECELTAGYKLFNDSQRITNELFIVKNIASMRKPAAYDVP |
| IMG_ | MAKKNKMKPRELREAQKKARQFKAAEINNNAAPAIAAMPAAQVIAPVAEKKKSSVKAAGMKSILV |
| 3300008520 | SENKMYITSFGKGNSAVLEYEVDNNDYNKTQLSSKDNSNIELCGVNEVNITFSSKHGFESGVEINTSN |
| SEQ ID NO: | PTHRSGESSPVRWDMLGLKSELEKRFFGKTFDDNIHIQLIYNILDIEKILAVYVTNIVYALNNMLGVK |
| 4820 | GSESHDDFIGYLSTNNTYDVFIDPDNSSLSDDKKANVRKSLSKFNVLLKTKRLGYFGLEEPKTKDNR |
| | VSEAYKKRVYHMLAIVGQIRQCVFHDKSGAKRFDLYSFINNIDPEYRDTLDYLVEERLKSINKDFIQG |
| | NKVNISLLIDMMKGYEADDIIRLYYDFIVLKSQKNLGFSIKKLREKMLEEYGFRFKDKQYDSVRSKM |
| | YKLMDFLLFCNYYRNDIAAGEALVRKLRFSMTDDEKEGLYADEAAKLWGKFRNDFENIADHMNG |
| | DVIKELGKADMDFDEKILDSEKKNASDLLYFSKMIYMLTL |
| IMG_ | MAKKNKMKPRELREAQKKARQFKAAEINNNAAPAIAAMPAAQVIAPVAEKKKSSVKAAGMKSILV |
| 3300008672 | SENKMYITSFGKGNSAVLEYEVDNNDYNKTQLSSKDNSNIELCGVNEVNITFSSKHGFESGVEINTSN |
| SEQ ID NO: | PTHRSGESSPVRWDMLGLKSELEKRFFGKTFDDNIHIQLIYNILDIEKILAVYVTNIVYALNNMLGVK |
| 4821 | GSESHDDFIGYLSTNNTYDVFIDPDNSSLSDDKKANVRKSLSKFNVLLKTKRLGYFGLEEPKTKDNR |
| | VSEAYKKRVYHMLAIVGQIRQCVFHDKSGAKRFDLYSFINNIDPEYRDTLDYLVEERLKSINKDFIQG |
| | NKVNISLLIDMMKGYEADDIIRLYYDFIVLKSQKNLGFSIKKLREKMLEEYGFRFKDKQYDSVRSKM |
| | YKLMDFLLFCNYYRNDIAAGEALVRKLRFSMTDDEKEGLYADEAAKLWGKFRNDFENIADHMNG |
| | DVIKELGKADMDFDEKILDSEKKNASDLLYFSKMIYMLTL |
| OWRJ01.1 | MAKKNKMKPRELREAQKKARQLKAAEINNNAIPAIAAMPAAEVIAPAEKKKSSVKAAGMKSILVSK |
| SEQ ID NO: | NKMYITSFGKGNSAVLEYEVDNNDYNKTQLSSKDNSNIELGDVNEVNITFSSKHGFGSGMKINTSNP |
| 4822 | THRSGESSPVRWDMLGLKSELEKRFFGKTFDDNIHIQLIYNILDIEKILAVYVTNIVYALNNMLGVKG |
| | SESHDDFIGYLSTNNIYDVFIDPDNSSLSDDKKANVRKSLSKFNVLLKTKRLGYFGLEEPKTKDNRVS |
| | EAYKKRVYHMLAIVGQIRQCVFHDKSGAKRFDLYSFINNIDPEYRDTLDYLVEERLKSINKDFIQGN |
| | KVNISLLIDMMKGYEADDIIRLYYDFIVLKSQKNLGFSIKKLREKMLEEYGFRFKDKQYDSVRSKMY |
| | KLMDFLLFCNYYRNDVVAGEALVRKLRFSMTDDEKEGIYADEAAKLWGKFRNDFENIADHMNGD |
| | VIKELGKADMDFDEKILDSEKKNASDLLYFSKMIYMLTYFLDGKEINDLLTTLISKFDNIKEFLKIMK |
| | SSAVDVECELTAGYKLFNDSQRITNELFIVKNIASMRKPAASAKLTMFRDALTILGIDDNITDDRISEIL |
| | KLKEKGKGIHGLRNFITNNVIESSRFVYLIKYANAQKIREVAKNEKVVMFVLGGIPDTQIERYYKSCV |
| | EFPDMNSSLEAKRSELARMIKNISFDDFKNVKQQAKGRENVAKERAKAVIGLYLTVMYLLVKNLVN |
| | VNARYVIAIHCLERDFGLYKEIIPELASKNLKNDYRILSQTLCELCDDRDESPNLFLKKNKRLRKCVE |
| | VDINNADSSMTRKYRNCIAHLTVVRELKEYIGDIRTVDSYFSIYHYVMQRCITKREMTQSKKRK |
| ULWL01.1 | VLSGIFVNAFSSKHGFESGVEINTSNPTHRSGESSPVRGDMLGLKSELEKRFFGKTFDDNIHIQLIYNIL |
| SEQ ID NO: | DIEKILAVYVTNIVYALNNMLGVKGSESHDDFIGYLSTNNTYDVFIDPDNSSLSDDKKANVRKSLSKF |
| 4823 | NVLLKTKRLGYFGLEEPKTKDNRVSEAYKKRVYHMLAIVGQIRQCVFHDKSGAKRFDLYSFINNIDP |
| | EYRDTLDYLVEERLKSINKDFIQGNKVNISLLIDMMKGYEADDIIRLYYDFIVLKSQKNLGFSIKKLRE |
| | KMLEEYGFRFKDKQYDSVRSKMYKLMDFLLFCNYYRNDIAAGEALVRKLRFSMTDDEKEGLYADE |
| | AAKLWGKFRNDFENIADHMNGDVIKELGKADMDFDEKILDSEKKNASDLLYFSKMIYMLTYFLDG |
| | KEINDLLTTLISKFDNIKEFLKIMKSSAVNVECELTAGYKLFNDSQRITNELFIVKNIASMRKPAASAK |
| | LTMFRDALTILGIDDKITDDRISEILKLKEKGKGIHGLRNFITNNVIESSRFVYLIKYANAQKIREVAEN |
| | EKVVMFVLGGIPDTQIERYYKSCVEFPDMNSSLEAKRSELARMIKNIRFDDFKNVKQQAKGRENVA |
| | KERAKAVIGLYLTVMYLLVKNLVNVNARYVIAIHCLERDFGLYKEIIPELASKNLKNDYRILSQTLCE |
| | LCDDRDESP |
| OLWE01.1 | VLSGIFVNAFSSKHGFESGVEINTSNPTHRSGESSPVRGDMLGLKSELEKRFFGKTFDDNIHIQLIYNIL |
| SEQ ID NO: | DIEKILAVYVTNIVYALNNMLGVKGSESHDDFIGYLSTNNIYDVFIDPDNSSLSDDKKANVRKSLSKF |
| 4824 | NVLLKTKRLGYFGLEEPKTKDNRVSEAYKKRVYHMLAIVGQIRQCVFHDKSGAKRFDLYSFINNIDP |
| | EYRDTLDYLVEERLKSINKDFIQGNKVNISLLIDMMKGYEADDIIRLYYDFIVLKSQKNLGFSIKKLRE |
| | KMLEEYGFRFKDKQYDSVRSKMYKLMDFLLFCNYYRNDVVAGEALVRKLRFSMTDDEKEGIYAD |
| | EAAKLWGKFRNDFENIADHMNGDVIKELGKADMDFDEKILDSEKKNASDLLYFSKMIYMLTYFLD |
| | GKEINDLLTTLISKFDNIKEFLKIMKSSAVDVECELTAGYKLFNDSQRITNELFIVKNIASMRKPAASA |
| | KLTMFRDALTILGIDDNITDDRISEILKLKEKGKGIHGLRNFITNNVIESSRFVYLIKYANAQKIREVAK |
| | NEKVVMFVLGGIPDTQIERYYKSCVEFPDMNSSLEAKRSELARMIKNISFDDFKNVKQQAKGRENV |
| | AKERAKAVIGLYLTVMYLLVKNLVNVNARYVIAIHCLERDFGLYKEIIPELASKNLKNDYRILSQTLC |
| | ELCDDRDESPNLFLKKNKRLRKC |
| OHUI01.1 | VIFMAKKNKMKPRELREAQKKARQLKAAEINNNAAPAIAAMPAAEVIAPAAEKKKSSVKAAGMKS |
| SEQ ID NO: | ILVSENKMYITSFGKGNSAVLEYEVDNNDYNQTQLSSKDNSNIQLGGVNEVNITFSSKHGFESGVEIN |
| 4825 | TSNPTHRSGESSPVRGDMLGLKSELEKRFFGKTFDDNIHIQLIYNILDIEKILAVYVTNIVYALNNMLG |
| | VKGSESHDDFIGYLSTNNIYDVFIDPDNSSLSDDKKANVRKSLSKFNALLKTKRLGYFGLEEPKTKDN |
| | RVSQAYKKRVYHMLAIVGQIRQCVFHDKSGAKRFDLYSFINNIDPEYRDTLDYLVEERLKSINKDFIE |
| | DNKVNISLLIDMMKGYEADDIIRLYYDFIVLKSQKNLGFSIKKLREKMLDEYGFRFKDKQYDSVRSK |
| | MYKLMDFLLFCNYYRNDIAAGESLVRKLRFSMTDDEKEGIYADEAAKLWGKFRNDFENIADHMNG |
| | DVIKELGKADMDFDEKILDSEKKNASDLLYFSKMIYMLTYFLDGKEINDLLTTLISKFDNIKEFLKIM |
| | KSSAVDVECELTAGYKLFNDSQRITNELFIVKNIASMRKPAASAKLTMFRDALTILGIDDKI |
| OKUN01.1 | VIFMAKKNKMKPRELREAQKKARQLKAAEINNNAAPAIAAMPAAEVIAPAAEKKKSSVKAAGMKS |
| SEQ ID NO: | ILVSENKMYITSFGKGNSAVLEYEVDNNDYNQTQLSSKDNSNIQLGGVNEVNITFSSKHGFESGVEIN |
| 4826 | TSNPTHRSGESSPVRGDMLGLKSELEKRFFGKTFDDNIHIQLIYNILDIEKILAVYVTNIVYALNNMLG |
| | VKGSESHDDFIGYLSTNNIYDVFIDPDNSSLSDDKKANVRKSLSKFNALLKTKRLGYFGLEEPKTKDN |
| | RVSQAYKKRVYHMLAIVGQIRQCVFHDKSGAKRFDLYSFINNIDPEYRDTLDYLVEERLKSINKDFIE |
| | DNKVNISLLIDMMKGYEADDIIRLYYDFIVLKSQKNLGFSIKKLREKMLDEYGFRFKDKQYDSVRSK |
| | MYKLMDFLLFCNYYRNDIAAGESLVRKLRFSMTDDEKEGIYADEAAKLWGKFRNDFENIADHMNG |
| | DVIKELGKADMDFDEKILDSEKKNASDLLYFSKMIYMLTYFLDGKEINDLLTTLISKFDNIKEFLKIM |
| | KSSAVDVECELTAGYKLFNDSQRITNELFIVKNIASMRKPAASAKLTMFRDALTILGIDDKITDDRISG |
| | ILKLKEKGKGIHGLRNFITNNVIESSRFVYLIKYANAQKIREVAKNEKVVMFVLGGIPDTQIERYYKS |
| | CVEFPDMNSSLGVKRSELARMIKNISFDDFKNVKQQAKGRENVAKERAKAVIGLYLTVMYLLVKNL |
| | VNVNARYVIAIHCLERDFGLYKEIIPELASKNLKNDYRILSQTLCELCDKSPNLFLKKNERL |
| OGRM01.1_2 | VIFMAKKNKMKPRELREAQKKARQLKAAEINNNAAPAIAAMPAAEAAEKKKSSVKAAGMKSILVS |
| SEQ ID NO: | ENKMYITSFGKGNSAVLEYEVDNNDYNQTQLSSKGSSNIELHGVNEVNITFSSKHGFESGVEINTSNP |
| 4827 | THRSGESSPVRWDMLGLKSELEKRFFGKTFDDNIHIQLIYNILDIEKILAVYVTNIVYALNNMLGVKG |
| | SESHDDFIGYLSTNNTYDVFIDPDNSSLSDDKKANVRKSLSKFNVLLKTKRLGYFGLEEPKTKDTRVS |
| | QAYKKRVYHMLAIVGQIRQCVFHDKSGAKRFDLYSFINNIDPEYRETLDYLVDERFDSINKGFIEGN |
| | KINISLLIDMMKGYEADDIIRLYYDFIVLKSQKNLGFSIKKLREKMLDEYGFRFKDKQYDPVRSKMY |
| | KLMDFLLFCNHYRNDVAAGEALVRKLRFSMTDDEKEGIYADEAAKLWGKFRNDFENIADHMNGD |
| | VIKELGKADMDFDEKILDSEKKNASDLLYFSKMIYMLTYFLDGKEINDLLTTLISKFDNIKEFLKIMK |
| | SSAVDVECELTAGYKLFNDSQRITNELFIVKNIASMRKPAASAKLTMFRDALTILGIDDNITDDRISEIL |
| | KLKEKGKGIHGLRNFITNNVIESSRFVYLIKYANAQKIREVAKNEKWMFVLGGIPDTQIERYYKSCV |
| | EFPDMNSSLEAKRSELARMIKNISFDDFKNVKQQAKGRENVAKERAKAVIGLYLTVMYLLVKNLVN |
| | VNARYVIAIHCLERDFGLYKEIIPELASKNLKNDYRILSQTLCEL |
| OBDE01.1 | MAKKNKMKPRELREAQKKARQLKAAEINNNAAPAIAAMPAAEAAAPAAEKKKSSVKAAGMKSIL |
| SEQ ID NO: | VSENKMYITSFGKGNSAVLEYEVDNNDYNQTQLSSKGSSNIELHGVNEVNITFSSKHGFESGVEINTS |
| 4828 | NPTHRSGESSPVRWDMLGLKSELEKRFFGKTFDDNIHIQLIYNILDIEKILAVYVTNIVYALNNMLGV |
| | KGSESHDDFIGYLSTNNTYDVFIDPDNSSLSDDKKANVRKSLSKFNVLLKTKRLGYFGLEEPKTKDT |
| | RVSQAYKKRVYHMLAIVGQIRQCVFHDKSGAKRFDLYSFINNIDPEYRETLDYLVDERFDSINKGFIE |
| | GNKINISLLIDMMKGYEADDIIRLYYDFIVLKSQKNLGFSIKKLREKMLDEYGFRFKDKQYDPVRSK |
| | MYKLMDFLLFCNHYRNDVAAGEALVRKLRFSMTDDEKEGIYADEAAKLWGKFRNDFENIADHMN |
| | GDVIKELGKADMDFDEKILDSEKKNASDLLYFSKMIYMLTYFLDGKEINDLLTTLISKFDNIKEFLKI |
| | MKSSAVDVECELTAGYKLFNDSQRITNELFIVKNIASMRKPAASAKLTMFRDALTILGIDDNITDDRI |
| | SEILKLKEKGKGIHGLRNFITNNVIESSRFVYLIKYANAQKIREVAKNEKVVMFVLGGIPDTQIERYYK |
| | SCVEFPDMNSSLEAKRSELARMIKNISFDDFKNVKQQAKGRENVAKERAKAVIGLYLTVMYLLVKN |
| | LVNVNARYVIAIHCLERDFGLYKEIIPELASKNLKNDYRILSQTLVNFVMIVMSRRICS |
| OWSQ01.1 | VIFMAKKNKMKPRELREAQKKARQLKAAEINNNAVPAIAAMPAAEVIAPAAEKKKSSVKAAGMKS |
| SEQ ID NO: | ILVSENKMYITSFGKGNSAVLEYEVDKVDNNNYNKTQLSSNDNSNIELRGVTKVNITFSSKHGLESG |
| 4829 | VEINTSNPTHRSGESSPVRWDMLGLKSELEKRFFGKTFDDNIHIQLIYNILDIEKILAVYVTNIVYALN |
| | NMLGVKGSESHDDFIGYLSTNNIYDVFIDPDNSSLSDDKKANVRKSLSKFNALLKTKRLGYFGLEEP |
| | KTKDNRVSQAYKKRVYHMLAIVGQIRQCVFHDKSGAKRFDLYSFINNIDPEYRDTLDYLVDERFDSI |
| | NKGFIEGNKINISLLIDMMKGYEADDIIRLYYDFIVLKSQKNLGFSIKKLREKMLDEYGFRFKDKQYD |
| | SVRSKMYKLMDFLLFCNYYRNDVVAGEALVRKLRFSMTDDEKEGIYADEAAKLWGKFRNDFENIA |
| | DHMNGDVTKELGKADMDFDEKILDSEKKNASDLLYFSKMIYMLTYFLDGKEINDLLTTLISKFDNIK |
| | EFLKIMKSSAVDVECELTAGYKLFNDSQRITNELFIVKNIASMRKPAASAKLTMFRDALTILGIDDKIT |
| | DDRISEILKLKEKGKGIHGLRNFITNNVIESSRFVYLIKYANAQKIREVAKNEKWMFVLGGIPDTQIE |
| | RYYKSCVEFPDMNSSLEVKRSELARMIKNISFDDFKNVKQQAKGRENVAKERAKAVIGLYLTVMYL |
| | LVKNLVNVNARYVIAIHCLERDFGLYKEIIPELASKNLKNDYRILSQTLCELCDERDKSPNLFLKKNE |
| | RLRKCVEVDINNADSIMTRKYRNCIAHLTVVREL |
| OOWK01.1 | MAKKNKMKPRELREAQKKARQFKAAEINNNAAPAIAAMPAAEAAAPAAEKKKSSVKAAGMKSIL |
| SEQ ID NO: | VSENKMYITSFGKGNSAVLEYEVDNNDYNKTQLSSKDNSNIELCDVNEVNITFSSKHGFESGVEINTS |
| 4830 | NPTHRSGESSPVRWDMLGLKSELEKRFFGKTFDDNIHIQLIYNILDIEKILAVYITNIVYALNNMLGVK |
| | GSESHDDFIGYLSTNNIYDVFIDPDNSSLSDDKKANVRKSLSKFNALLKTKRLGYFGLEEPKTKDNRV |
| | SEAYKKRVYHMLAIVGQIRQCVFHDLSEHSEYDLYSFIDNSKKVYRECRETLDYLVDERFDSINKGFI |
| | QGNKVNISLLIDMMKGYEADDIIRLYYDFIVLKSQKNLGFSIKKLREKMLEEYGFRFKDKQYDSVRS |
| | KMYKLMDFLLFCNYYRNDVVAGEALVRKLRFSMTDDEKEGIYADEAEKLWGKFRNDFENIADHM |
| | NGDVIKELGKADMDFDEKILDSEKKNASDLLYFSKMrYMLTYFLDGKEINDLLTTLISKFDNIKEFLK |
| | IMKSSAVDVECELTAGYKLFNDSQRITNELFIVKNIASMRKPAASAKLTMFRDALTILGIDDKITDDRI |
| | SEILKLKEKGKGIHGLRNFVTNNVIESSRFVYLIKYANAQKIREVAKNEKWMFVLGGILTRR |
| OPSZ01.1 | MAKKNKMKPRELREAQKKARQFKAAEINNNAAPAIAAMPAAEVIAPVAEKKKSSVKAAGMKSILV |
| SEQ ID NO: | SENKMYITSFGKGNSAVLEYEVDNNDYNKTQLSSKDNSNIELCGVNEVNITFSSKHGFESGVEINTSN |
| 4831 | PTHRSGESSPVRWDMLGLKSELEKRFFGKTFDDNIHIQLIYNILDIEKILAVYVTNIVYALNNMLGEG |
| | DESNYDFMGYLSTFNTYKVFTNPNGSTLSDDKKENIRKSLSKFNALLKTKRLGYFGLEEPKTKDTRV |
| | LEAYKKRVYHMLAIVGQIRQCVFHDLSEHSEYDLYSFIDNSKKVYRECRETLDYLVDERFDSINKGFI |
| | QGNKVNISLLIDMMKGYEADDIIRLYYDFIVLKSQKNLGFSIKKLREKILDEYGFRFKDKQYDSVRSK |
| | MYKLMDFLLFCNYYRNDIAAGESLVRKLRFSMTDDEKEGIYADEAAKLWGKFRNDFENIADHMNG |
| | DVIKELGKADMDFDEKILDSEKKNASDLLYFSKMIYMLTYFLDGKEINDLLTTLISKFDNIKEFLKIM |
| | KSSAIDVECELTAGYKLFNDSQRITNELFIVKNIASMRKPAASAKLTMFRDALTILGIDDKITDDRISEI |
| | LKLKEKGKGIHGLRNFITNNVIESSRFVYLIKYANAQKIREVAKNEKWMFVLGGIPDTQIERYYKSC |
| | VEFPDMNSSLEAKRSELARMIKNIRFDDFKNVKQQAKGRENVAKERAKAVIGLYLTVMYLLVKNL |
| | VNVNARYVIAIHCLERDFGLYKEIIPELASKNLKNDYRILSQTLCELCDNGDESPNLFLKKNKRLRKC |
| | VEVDINNADNFVSATQGNLF |
| ULRO01.1 | VIFMAKKNKMKPRELREAQKKARQLKAAEINNNAVPAIAAMPAAEAAAPAAEKKKSSVKAAGMK |
| SEQ ID NO: | SILVSENKMYITSFGKGNSAVLEYEVDNNDYNKTQLSSKDNSNIELCDVDEVNITFSSKHGFESGVEI |
| 4832 | NTSNPTHRSGESSPVRWDMLGLKSELEKRFFGKTFDDNIHIQLIYNILDIEKILAVYVTNIVYALNNML |
| | GEGDESNYDFMGYLSTFNTYKVFTNPNGSTLSDDKKENIRKSLSKFNALLKTKRLGYFGLEEPKTKD |
| | TRVLEAYKKRVYHMLAIVGQIRQCVFHDLSEHSEYDLYSFIDNSKKVYRECRETLDYLVDERFDSIN |
| | KGFIQGNKVNISLLIDMMKGYEADDIIRLYYDFIVLKSQKNLGFSIKKLREKMLEEYGFRFKDKQYDS |
| | VRSKMYKLMDFLLFCNYYRNDVVAGEALVRKLRFSMTDDEKEGIYADEAEKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVDVECELTAGYKLFNDSQRITNELFIV |
| OJAG01.1_2 | MAKKNKMKPRELREAQKKARQLKAAEINNNAVPAIAAMPAAEAAAPAAEKKKSSVKAAGMKSIL |
| SEQ ID NO: | VSENKMYITSFGKGNSAVLEYEVDNNDYNKTQLSSKDNSNIELCDVGKVNITFSSRRGFESGVEINTS |
| 4833 | NPTHRSGESSSVRGDMLGLKSELEKRFFGKNFDDNIHIQLIYNILDIEKILAVYVTNIVYALNNMLGE |
| | GDESNYDFMGYLSTFNTYKVFTNPNGSTLSDDKKENIRKSLSKFNALLKTKRLGYFGLEEPKTKDTR |
| | ASEAYKKRVYHMLAIVGQIRQCVFHDKSGAKRFDLYSFINNIDPEYRETLDYLVDERFDSINKGFIQG |
| | NKVNISLLIDMMKGYEADDIIRLYYDFIVLKSQKNLGFSIKKLREKMLDEYGFRFKDKQYDSVRSKM |
| | YKLMDFLLFCNYYRNDIAAGESLVRKLVFQ |
| UMGZ01.1 | VIFMAKKNKMKPRELREAQKKARQLKAAEINNNAAPAIAAMPAAEAAAPAAEKKKSSVKAAGMK |
| SEQ ID NO: | SILVSENKMYITSFGKGNSAVLEYEVDNNDYNKTQLSSKDNSNIELCDVGKVNITFSSRRGFESGVEI |
| 4834 | NTSNPTHRSGESSSVRGDMLGLKSELEKRFFGKNFDDNIHIQLIYNILDIEKILAVYVTNIVYALNNML |
| | GEGDESNYDFMGYLSTFNTYKVFTNPNGSTLSDDKKENIRKSLSKFNALLKTKRLGYFGLEEPKTKD |
| | TRASEAYKKRVYHMLAIVGQIRQCVFHDKSGAKRFDLYSFINNIDPEYRETLDYLVDERFDSINKGFI |
| | QGNKVNISLLIDMMKGYEADDIIRLYYDFIVLKSQKNLGFSIKKLREKMLDEYGFRFKDKQYDSVRS |
| | KMYKLMDFLLFCNYYRNDIAAGESLVRKLRFSMTDDEKEGIYADEAAKLWGKFRNDFENIADHMN |
| | GDVIKELGKADMDFDEKILDSEKKNASDLLYFSKMIYMLTYFLDGKEINDLLTTLISKFDNIKEFLKI |
| | MKSSAVDVECELTAGYKLFNDSQRITNELFIVKNIASMRKPAASAKLTMFRDALTILGIDDKITDDRI |
| | SEILKLKEKGKGIHGLRNFITNNVIESSRFVYLIKYANAQKIREVAKNEKVVMFVLGGIPDTQIERYYK |
| | SCVEFPDMNSSLKVKRSELARMIKNIRFDDFKNVKQQAKGRENVAKERAKAVIGLYLTVMYLLVK |
| | NLVNVNARYVIAIHCLERDFGLYKEIIPELASKNLKNDYRILSQTLCELCDKSPNLFL |
| UAHN01.1 | VIFMAKKNKMKPRELREAQKKARQLKAAEINNNAVPAIAAMPAAEAAAPAAEKKKSSVKAAGMK |
| SEQ ID NO: | SILVSENKMYITSFGKGNSAVLEYEVDNNDYNKTQLSSKDNSNIELCDVGKVNITFSSRRGFESGVEI |
| 4835 | NTSNPTHRSGESSSVRGDMLGLKSELEKRFFGKNFDDNIHIQLIYNILDIEKILAVYVTNIVYALNNML |
| | GEGDESNYDFMGYLSTFNTYKVFTNPNGSTLSDDKKENIRKSLSKFNALLKTKRLGYFGLEEPKTKD |
| | TRASEAYKKRVYHMLAIVGQIRQCVFHDKSGAKRFDLYSFINNIDPEYRETLDYLVDERFDSINKGFI |
| | QGNKVNISLLIDMMKGYEADDIIRLYYDFIVLKSQKNLGFSIKKLREKMLDEYGFRFKDKQYDSVRS |
| | KMYKLMDFLLFCNYYRNDIAAGESLVRKLRFSMTNDEKEGIYADEAAK |
| OHAE01.1 | MAKKNKMKPRELREAQKKARQLKAAEINNNAAPAIAAMPAAEVIAPAAEKKKSSVKAAGMKSILV |
| SEQ ID NO: | SENKMYITSFGKGNSAVLEYEVDNNDYNKTQLSSKDNSNIELCGVNEVNITFSSKHGFESGVEINTSN |
| 4836 | PTHRSGESSPVRWDMLGLKSELEKRFFGKTFDDNIHIQLIYNILDIEKILAVYVTNIVYALNNMLGEG |
| | DESNYDFMGYLSTFNTYKVFTNPNGSTLSDDKKENIRKSLSKFNALLKTKRLGYFGLEEPKTKDTRA |
| | SEAYKKRVYHMLAIVGQIRQCVFHDKSGAKRFDLYSFINNIDPEYRDTLDYLVDERFDSINKGFIQGN |
| | KVNISLLIDMMKGYEVDDIIRLYYDFIVLKSQKNLGFSIKKLREKMLDEYGFRFKDKQYDSVRSKMY |
| | KLMDFLLFCNYYRNDVVAGETLVRKLRFSMTDDEKEGIYADEAS |
| OWRW01.1_2 | MAKKNKMKPRELREAQKKARQLKAAEINNNAAPAIAAMPAAEVIAPAAEKKKSSVKAAGMKSILV |
| SEQ ID NO: | SENKMYITSFGKGNSAVLEYEVDNNDYNKTQLSSKDNSNIELCGVNEVNITFSSKHGFESGVEINTSN |
| 4837 | PTHRSGESSPVRWDMLGLKSELEKRFFGKTFDDNIHIQLIYNILDIEKILAVYVTNIVYALNNMLGEG |
| | DESNYDFMGYLSTFNTYKVFTNPNGSTLSDDKKENIRKSLSKFNALLKTKRLGYFGLEEPKTKDTRA |
| | SEAYKKRVYHMLAIVGQIRQCVFHDKSGAKRFDLYSFINNIDPEYRDTLDYLVDERFDSINKGFIQGN |
| | KVNISLLIDMMKGYEVDDIIRLYYDFIVLKSQKNLGFSIKKLREKMLDEYGFRFKDKQYDSVRSKMY |
| | KLMDFLLFCNYYRNDVVAGETLVRKLRFSMTDDEKEGIYADEAS |
| UPDL01.1 | MAKKNKMKPRELREAQKKARQLKAAEINNNAAPAIAAMPAAEVIAPAAEKKKSSVKAAGMKSILV |
| SEQ ID NO: | SENKMYITSFGKGNSAVLEYEVDNNDYNKTQLSSKDNSNIELCGVNEVNITFSSKHGFESGVEINTSN |
| 4838 | PTHRSGESSPVRWDMLGLKSELEKRFFGKTFDDNIHIQLIYNILDIEKILAVYVTNIVYALNNMLGEG |
| | DESNYDFMGYLSTFNTYKVFTNPNGSTLSDDKKENIRKSLSKFNALLKTKRLGYFGLEEPKTKDTRA |
| | SEAYKKRVYHMLAIVGQIRQCVFHDKSGAKRFDLYSFINNIDPEYRDTLDYLVDERFDSINKGFIQGN |
| | KVNISLLIDMMKGYEVDDIIRLYYDFIVLKSQKNLGFSIKKLREKMLDEYGFRFKDKQYDSVRSKMY |
| | KLMDFLLFCNYYRNDVVAGETLVRKLRFSMTDDEKEGIYADEAS |
| OGHH01.1_2 | MAKKNKMKPRELREAQKKARQLKAAEINNNAVPAIAAMPAAEVIAPAAEKKKSSVKAAGMKSILV |
| SEQ ID NO: | SENKMYITSFGKGNSAVLEYEVDNNDYNQTQLSSKDNSNIELGGVNEVNITFSSKHGFESGVEINTSN |
| 4839 | PTHRSGESSPVRWDMLGLKSELEKRFFGKTFDDNIHIQLIYNILDIEKILAVYVTNIVYALNNMLGEG |
| | DESNYDFMGYLSTFNTYKVFTNPNGSTLSDDKKENIRKSLSKFNALLKTKRLGYFGLEEPKTKDTRA |
| | SEAYKKRVYHMLAIVGQIRQCVFHDKSGAKRFDLYSFINNIDPEYRDTLDYLVDERFDSINKGFIQGN |
| | KVNISLLIDMMKGYEVDDIIRLYYDFIVLKSQKNLGFSIKKLREKMLDEYGFRFKDKQYDSVRSKMY |
| | KLMDFLLFCNYYRNDVVAGETLVRKLRFSMTDDEKEGIYADEAS |
| OURZ01.1 | MAKKNKMKPRELREAQKKARQLKAAEINNNAAPAIAAMPAAEVIAPAAEKKKSSVKAAGMKSILV |
| SEQ ID NO: | SKNKMYITSFGKGNSAVLEYEVDKVDNNNYNKTQLSSKDNSNIELGDVNEVNITFSSKHGFESGVEI |
| 4840 | NTSNPTHRSGESSPVRWDMLGLKSELEKRFFGKTFDDNIHIQLIYNILDIEKILAVYVTNIVYALNNML |
| | GEGEDSNYDFMGYLSTFNTYKVFTNPNGSTLSDDKKKNIRKSLSKFNALLKTKRLGYFGLEEPKTKD |
| | TRASEAYKKRVYHMLAIVGQIRQCVFHDKSGAKRFDLYSFINNIDPEYRDTLDYLVDERFDSINKGFI |
| | QGNKVNISLLIDMMKGYEADDIIRLYYDFIVLKSQKNLGFS |
| OGOM01.1 | MAKKNKMKPRELREAQKKARQLKAAEINNNAVPAIAAMPAAEVIAPAAEKKKSSVKAAGMKSILV |
| SEQ ID NO: | SENKMYITSFGKGNSAVLEYEVDKVDNNNYNKTQLSSKDNSNIELGDVNEVNITFSSKRGNESGVEI |
| 4841 | NTSNPTHRSGESSPVRWDMLGLKSELEKRFFGKTFDDNIHIQLIYNILDIEKILAVYVTNIVYALNNML |
| | GIKKSESYDDFMGYLSARNTYEVFTHPDKSNLSDKAKGNIKKSFSTFNDLLKTKRLGYFGLEEPKTK |
| | DTRVSQAYKKRVYHMLAIVGQIRQSVFHDKSSKLDEDLYSFIDIIDPEYRETLDYLVDERFDSINKGFI |
| | QGNKVNISLLIDMMKGYEADDIIRLYYDFIVLKSQKNLGFFYQKAS |
| UPFO01.1 | MAKKNKMKPRELREAQKKARQLKAAEINNNAVPAIAAMPAAEVIAPAAEKKKSSVKAAGMKSILV |
| SEQ ID NO: | SENKMYITSFGKGNSAVLEYEVDKVDNNNYNKTQLSSKDNSNIELGDVNEVNITFSSKRGNESGVEI |
| 4842 | NTSNPTHRSGESSPVRWDMLGLKSELEKRFFGKTFDDNIHIQLIYNILDIEKILAVYVTNIVYALNNML |
| | GIKKSESYDDFMGYLSARNTYEVFTHPDKSNLSDKAKGNIKKSFSTFNDLLKTKRLGYFGLEEPKTK |
| | DTRVSQAYKKRVYHMLAIVGQIRQSVFHDKSSKLDEDLYSFIDIIDPEYRETLDYLVDERFDSINKGFI |
| | QGNKVNISLLIDMMKGYEADDIIRLYYDFIVLKSQKNLGFFYQKAS |
| OVZV01.1 | MAKKNKMKPRELREAQKKARQLKAAEINNNAVPAIAAMPAAEVIAPAAEKKKSSVKAAGMKSILV |
| SEQ ID NO: | SENKMYITSFGKGNSAVLEYEVDKVDNNNYNKTQLSSKDNSNIELGDVNEVNITFSSKRGNESGVEI |
| 4843 | NTSNPTHRSGESSPVRWDMLGLKSELEKRFFGKTFDDNIHIQLIYNILDIEKILAVYVTNIVYALNNML |
| | GIKKSESYDDFMGYLSARNTYEVFTHPDKSNLSDKAKGNIKKSFSTFNDLLKTKRLGYFGLEEPKTK |
| | DTRVSQAYKKRVYHMLAIVGQIRQSVFHDKSSKLDEDLYSFIDIIDPEYRETLDYLVDERFDSINKGFI |
| | QGNKVNISLLIDMMKGYEADDIIRLYYDFIVLKSQKNLGFFYQKAS |
| OGGK01.1 | MAKKNKMKPRELREAQKKARQLKAAEINNNAVPAIAAMPAAEVIAPAAEKKKSSVKAAGMKSILV |
| SEQ ID NO: | SENKMYITSFGKGNSAVLEYEVDKVDNNNYNKTQLSSKDNSNIELGDVNEVNITFSSKRGNESGVEI |
| 4844 | NTSNPTHRSGESSPVRWDMLGLKSELEKRFFGKTFDDNIHIQLIYNILDIEKILAVYVTNIVYALNNML |
| | GIKKSESYDDFMGYLSARNTYEVFTHPDKSNLSDKAKGNIKKSFSTFNDLLKTKRLGYFGLEEPKTK |
| | DTRVSQAYKKRVYHMLAIVGQIRQSVFHDKSSKLDEDLYSFIDIIDPEYRETLDYLVDERFDSINKGFI |
| | QGNKVNISLLIDMMKGYEADDIIRLYYDFIVLKSQKNLGFFYQKAS |
| OLQT01.1 | MAKKNKMKPRELREAQKKARQLKAAEIKNNAVPAIAAMPAAEAAAPAVEKKKSSVKAAGMKSIL |
| SEQ ID NO: | VSENKMYITSFGKGNSAVLEYEVDKVDNNNYNKTQLSSKDNSNIELGDVNEVNITFSSKHGFESGVE |
| 4845 | INTSNPTHRSGESSPVRWDMLGLKSELEKRFFGKTFDDNIHIQLIYNILDIEKILAVYVTNIVYALNNM |
| | LGIKDSESYDDFMGYLSARNTYEVFTHPDKSNLSDKVKGNINKVKCNIKKSFSTFNDLLKTKRLGYF |
| | GLEEPKTKDTRVSQAYKKRVYHMLAIVGQIRQSVFHDKSSKLDEDLYSFIDIIDSEYRETLDYLVDER |
| | FDSINKGFIQGNKVNISLLIDMMKGYEADDIIRLYYDFIVLKSQKNLGFSIKKLREKILDEYGFRFKDK |
| | QYDSVRSKMYKLMDFLLFCNYYRNDIAAGESLVRKLRFSMTDDEKEGIYADEAAKLWGKFRNDFE |
| | NIADHMNGDVIKELGKADMDFDEKILDSEKKNASDLLYFSKMIYMLTYFLDGKEINDLLTTLISKFD |
| | NIKEFLKIMKSSAVNVECELTAGYKLFNDSQRITNELFIVKNIASMRKPAASAKLTMFRDALTILGIDD |
| | NITDDRISEILGHTWSEEFHNKQCYRVLSVCIPYQVCERSEDKRSG |
| USUN01.1 | MAKKNKMKPRELREAQKKARQLKAAEINNNAAPAIAAMPAAEAAAPAAEKKKSSVKAAGMKSIF |
| SEQ ID NO: | VSENKMYITSFGKGNSAVLEYEVDKVDNDNYNKTQLSSEDSSNIELCGVNEVNITFSSKHGFGSGVEI |
| 4846 | NTSNPTHRSGESSPVRWDMLGLKSELEKRFFGKTFDDNIHIQLIYNILDIEKILAVYVTNIVYALNNML |
| | GIKKSESYDDFMGYLSARNTYEVFTHPDKSNLSDKAKGNIKKSFSTFNDLLKTKRLGYFGLEEPKTK |
| | DTRVSQAYKKRVYHMLAIVGQIRQSVFHDKSSKLDEDLYSFIDIIDSEYRETLDYLVDE |
| OXWC01.1 | MAKKNKMKPRELREAQKKARQLKAAEINNNAAPAIAAMPAAQVIAPAAEKKKSSVKAAGMKSILV |
| SEQ ID NO: | SENKMYITSFGKGNSAVLEYEVDKVDNNNYNKTQLSSKDNSNIELGDVNEVNITFSSKHGFESGVEI |
| 4847 | NTSNPTHRSGESSSVRGDMLGLKSELEKRFFGKTFDDNIHIQLIYNILDIEKILAVYVTNIVYALNNML |
| | GVKGSESYDDFMGYLSAQNTYYIFTHPDKSNLSDKVKGNIKKSLSKFNDLLKTKRLGYFGLEEPKTK |
| | DKRVSEAYKKRVYHMLAIVGQIRQSVFHDKSNELDEYLYSFIDIIDSEYRDTLDYLVDERFDSINKGF |
| | VQGNKVNISLLIDMMKGYEADDIIRLYYDFIVLKSQKNLGFSIKKLREKMLDEYGFRFKDKQYDSVR |
| | SKMYKLMDFLLFCNYYRNDIAAGEALVRKLRFSMTDDEKEGLYADEAAKLWGKFRNDFENIADH |
| | MNGDVTKELGKADMDFDEKILDSEKKNASDLLYFSKMIYMLTYFLDGKEINDLLTTLISKFDNIKEFL |
| | KIMKSSAVNVECELTAGYKLFNDSQRITNELFIVKNIASMRKPAASAKLTMFRDALTILGIDDNITDD |
| | RISEILKLKEKGKGIHGLRNFITNNVTESSRFVYLIKYANAQKIREVAKNEKWMFVLGGIPDTQIERY |
| | YKSCVEFPDMNSSLEVKRSELARMIKNIRFDDFKNVKQQAKGRENVAKERAKAVIGLYLTVMYLLV |
| | KNLVNVNARYVIAIHCLERDFGLYKEIIPELASKNLKNDYRILSQTLCELCDNRDESPNLFLKKNKRL |
| | RKCVEVDINNADSSMTRKYRNCIAHLTVVRELKEYIGDIRTVDSYFSIYHYVM |
| OZPS01.1 | MAKKNKMKPRELREAQKKARQLKAAEINNNAAPAIAAMPAAQVIAPAAEKKKSSVKAAGMKSILV |
| SEQ ID NO: | SENKMYITSFGKGNSAVLEYEVDKVDNNNYNKTQLSSKDNSNIELGDVNEVNITFSSKHGFESGVEI |
| 4848 | NTSNPTHRSGESSSVRGDMLGLKSELEKRFFGKTFDDNIHIQLIYNILDIEKILAVYVTNIVYALNNML |
| | GVKGSESYDDFMGYLSAQNTYYIFTHPDKSNLSDKVKGNIKKSLSKFNDLLKTKRLGYFGLEEPKTK |
| | DKRVSEAYKKRVYHMLAIVGQIRQSVFHDKSNELDEYLYSFIDIIDSEYRDTLDYLVDERFDSINKGF |
| | VQGNKVNISLLIDMMKGYEADDIIRLYYDFIVLKSQKNLGFSIKKLREKMLDEYGFRFKDKQYDSVR |
| | SKMYKLMDFLLFCNYYRNDIAAGEALVRKLRFSMTDDEKEGLYADEAAKLWGKFRNDFENIADH |
| | MNGDVTKELGKADMDFDEKILDSEKKNASDLLYFSKMIYMLTYFLDGKEINDLLTTLISKFDNIKEFL |
| | KIMKSSAVNVECELTAGYKLFNDSQRITNELFIVKNIASMRKPAASAKLTMFRDALTILGIDDNITDD |
| | RISEILKLKEKGKGIHGLRNFITNNVTESSRFVYLIKYANAQKIREVAKNEKWMFVLGGIPDTQIERY |
| | YKSCVEFPDMNSSLEVKRSELARMIKNIRFDDFKNVKQQAKGRENVAKERAKAVIGLYLTVMYLLV |
| | KNLVNVNARYVIAIHCLERDFGLYKEIIPELASKNLKNDYRILSQTLCELCDNRDESPNLFLKKNKRL |
| | RKCVEVDINNADSSMTRKYRNCIAHLTVVRELKEYIGDIRTVDSYFSIYHYVM |
| OWFV01.1 | MAKKNKMKPRELREAQKKARQLKAAEINNNAAPAIAAMPAAQVIAPAAEKKKSSVKAAGMKSILV |
| SEQ ID NO: | SENKMYITSFGKGNSAVLEYEVDKVDNNNYNKTQLSSKDNSNIELGDVNEVNITFSSKHGFESGVEI |
| 4849 | NTSNPTHRSGESSSVRGDMLGLKSELEKRFFGKTFDDNIHIQLIYNILDIEKILAVYVTNIVYALNNML |
| | GVKGSESYDDFMGYLSAQNTYYIFTHPDKSNLSDKVKGNIKKSLSKFNDLLKTKRLGYFGLEEPKTK |
| | DKRVSEAYKKRVYHMLAIVGQIRQSVFHDKSNELDEYLYSFIDIIDSEYRDTLDYLVDERFDSINKGF |
| | VQGNKVNISLLIDMMKGYEADDIIRLYYDFIVLKSQKNLGFSIKKLREKMGVLF |
| ORWL01.1 | MAKKNKMKPRELREAQKKARQLKAAEINNNAAPAIAAMPAAEVIAPVAEKKKSSVKAAGMKSILV |
| SEQ ID NO: | SENKMYITSFGKGNSAVLEYEVDNNDYNKTQLSSKDNSNIELGDVNEVNITFSSKHGFGSGVEINTS |
| 4850 | NPTHRSGESSSVRGDMLGLKSELEKRFFGKTFDDNIHIQLIYNILDIEKILAVYVTNIVYALNNMLGV |
| | KGSESYDDFMGYLSAQNTYYIFTHPDKSNLSDKVKGNIKKSLSKFNDLLKTKRLGYFGLEEPKTKDK |
| | RVSEAYKKRVYHMLAIVGQIRQSVFHDKSNELDEYLYSFIDIIDSEYRDTLDYLVDERFDSINKGFVQ |
| | GNKVNISLLIDMMKGYEADDIIRLYYDFIVLKSQKNLGFSIKKLREKM |
| OVMH01.1 | VIFMAKKNKMKPRELREAQKKARQLKAAEINNNAVPAIAAMPAAEVIAPAAEKKKSSVKAAGMKS |
| SEQ ID NO: | ILVSKNKMYIISFGKGNSAVLEYEVDKVDDNDYNKTQLSSKDNSNIELGDVNEVNITFSSKHGFGSG |
| 4851 | VEINTSNPTHRSGESSPVRWDMLGLKSELEKRFFGKTFDDNIHIQLIYNILDIEKILAVYVTNIVYALN |
| | NMLGVKGSESYDDFMGYLSAQNTYYIFTHPDKSNLSDKVKGNIKKSLSKFNDLLKTKRLGYFGLEE |
| | PKTKDKRVSEAYKKRVYHMLAIVGQIRQSVFHDKSNELDEYLYSFIDIIDSEYRDTLDYLVDERFDSI |
| | NKGFVQGNKVNISLLIDMMKGYEADDIIRLYYDFIVLKSQKNLGFSIKKLREKMLDEYGFRFKDKQY |
| | DSVRSKMYKLMDFLLFCNYYRNDVVAGEALVRKLRFSMTDDEKEGIYADEAEKLWVKFRNDFENI |
| | ADHMNGDVTKELGKADMDFDEKILDSEKKNASDLLYFSKMIYMLTYFLDGKEINDLLTTLISKFDNI |
| | KEFLKIMKSSAVDVECELTAGYKLFNDSQRITNELFIVKNIASMRKPAASAKLTMFRDALTILGIDDNI |
| | TDDRISEILKLKEKGKGIHGLRNFITNNVTESSRFVYLIKYANAQKIREVAKNEKWMFVLGGIPDTQI |
| | ERYYKSCVEFPDMNSSLEAKRSELARMIKNISFDDFKNVKQQAKGRENVAKERAKAVIGLYLTVMY |
| | LLVKNLVNVNARYVIAIHCLERDFGLYKEIIPELASKNLKNDYRILSQTLCELCDDRDKSPNLFLKKN |
| | KRLRKCVEVDINNADSSMTRKYRNCIAHLTVVRELKEYIGDIRTVDSYFSIYHYVMQRCITKREDDT |
| | KQEDKIKYEDDLLKNHGYTKDFVKAL |
| OODG01.1 | VIFMAKKNKMKPRELREAQKKARQLKAAEINNNAVPAIAAMPAAEVIAPAAEKKKSSVKAAGMKS |
| SEQ ID NO: | ILVSKNKMYIISFGKGNSAVLEYEVDKVDDNDYNKTQLSSKDNSNIELGDVNEVNITFSSKHGFGSG |
| 4852 | VEINTSNPTHRSGESSPVRWDMLGLKSELEKRFFGKTFDDNIHIQLIYNILDIEKILAVYVTNIVYALN |
| | NMLGVKGSESYDDFMGYLSAQNTYYIFTHPDKSNLSDKVKGNIKKSLSKFNDLLKTKRLGYFGLEE |
| | PKTKDKRVSEAYKKRVYHMLAIVGQIRQSVFHDKSNELDEYLYSFIDIIDSEYRDTLDYLVDERFDSI |
| | NKGFVQGNKVNISLLIDMMKGYEADDIIRLYYDFIVLKSQKNLGFSIKKLREKMLDEYGFRFKDKQY |
| | DSVRSKMYKLMDFLLFCNYYRNDVVAGEALVRKLRFSMTDDEKEGIYADEAEKLWVKFRNDFENI |
| | ADHMNGDVTKELGKADMDFDEKILDSEKKNASDLLYFSKMIYMLTYFLDGKEINDLLTTLISKFDNI |
| | KEFLKIMKSSAVDVECELTAGYKLFNDSQRITNELFIVKNIASMRKPAASAKLTMFRDALTILGIDDNI |
| | TDDRISEILKLKEKGKGIHGLRNFITNNVTESSRFVYLIKYANAQKIREVAKNEKWMFVLGGIPDTQI |
| | ERYYKSCVEFPDMNSSLEAKRSELARMIKNISFDDFKNVKQQAKGRENVAKERAKAVIGLYLTVMY |
| | LLVKNLVNVNARYVIAIHCLERDFGLYKEIIPELASKNLKNDYRILSQTLCELCDDRDKSPNLFLKKN |
| | KRLRKCVEVDINNADSSMTRKYRNCIAHLTVVRELKEYIGDIRTVDSYFSIYHYVMQRCITKREDDT |
| | KQEDKIKYEDDLLKNHGYTKDFVKAL |
| UXRU01.1 | MAKKNKMKPRELREAQKKARQLKAAEINNNAAPAIAAMPAAQVIAPAAEKKKSSVKAAGMKSILV |
| SEQ ID NO: | SENKMYITSFGKGNSAVLEYEVDKVDNNNYNKTQLSSKDNSNIELGDVNEVNITFSSKHGFESGVEI |
| 4853 | NTSNPTHRSGESSPVRWDMLGLKSELEKRFFGKTFDDNIHIQLIYNILDIEKILAVYVTNIVYALNNML |
| | GIKGSESYDDFMGYLSARNTYEVFTHPDKSNLSDKVKGNIKKSLSKFNDLLKTKRLGYFGLEEPKTK |
| | DTRVSQAYKKRVYHMLAIVGQIRQSVFHDKSSKLDEDLYSFIDIIDSEYRETLDYLVDERFDSINKGFI |
| | QGNKVNISLLIDMMKGYEADDIIRLYYDFIVLKSQKNLGFSIKKLREKMLDEYGFRFKDKQYDSVRS |
| | KMYKLMDFLLFCNYYRNDVVAGEALVRKLRFSMTDDEKEGIYADEASKLWGKFRNDFENIADHM |
| | NGDVIKELGKADMDFDEKILDSEKKNASDLLYFSKMIYMLTYFLDGKEINDLLTTLISKFDNIKEFLK |
| | IMKSSAVDVECELTAGYKLFNDSQRITNELFIVKNIASMRKPASSAKLTMFRDALTILGIDDNITDDRI |
| | SEILKLKEKGKGIHGLRNFITNNVTESSRFVYLIKYANAQKIRKVAKNEKWMFVLGGIPDTQIERYY |
| | KSCVEFPDMNSSLEVKRSELARMIKNISFDDFKNVKQQAKGRENVAKERAKAVIGLYLTVMYLLVK |
| | NLVNVNARYVIAIHCLERDFGLYKEIIPELASKNLKNDYRILSQTLCELCDKSPNLFLKKNRRLRKCV |
| | EVDINNADSSMTRKYRNCIAHLTVVRELKEYIGDIRTVDSYFSIYHYVM |
| ULUP01.1 | MAKKNKMKPRELREAQKKARQLKAAEINNNAAPAIAAMPAAQVIAPAAEKKKSSVKAAGMKSILV |
| SEQ ID NO: | SENKMYITSFGKGNSAVLEYEVDKVDNNNYNKTQLSSKDNSNIELGDVNEVNITFSSKHGFESGVEI |
| 4854 | NTSNPTHRSGESSPVRWDMLGLKSELEKRFFGKTFDDNIHIQLIYNILDIEKILAVYVTNIVYALNNML |
| | GIKGSESYDDFMGYLSARNTYEVFTHPDKSNLSDKVKGNIKKSLSKFNDLLKTKRLGYFGLEEPKTK |
| | DTRVSQAYKKRVYHMLAIVGQIRQSVFHDKSSKLDEDLYSFIDIIDSEYRETLDYLVDERFDSINKGFI |
| | QGNKVNISLLIDMMKGYEADDIIRLYYDFIVLKSQKNLGFSIKKLREKMLDEYGFRFKDKQYDSVRS |
| | KMYKLMDFLLFCNYYRNDVVAGEALVRKLRFSMTDDEKEGIYADEASKLWGKFRNDFENIADHM |
| | NGDVIKELGKADMDFDEKILDSEKKNASDLLYFSKMIYMLTYFLDGKEINDLLTTLISKFDNIKEFLK |
| | IMKSSAVDVECELTAGYKLFNDSQRITNELFIVKNIASMRKPASSAKLTMFRDALTILGIDDNITDDRI |
| | SEILKLKEKGKGIHGLRNFITNNVTESSRFVYLIKYANAQKIRKVAKNEKWMFVLGGIPDTQIERYY |
| | KSCVEFPDMNSSLEVKRSELARMIKNISFDDFKNVKQQAKGRENVAKERAKAVIGLYLTVMYLLVK |
| | NLVNVNARYVIAIHCLERDFGLYKEIIPELASKNLKNDYRILSQTLCELCDKSPNLFLKKNRRLRKCV |
| | EVDINNADSSMTRKYRNCIAHLTVVRELKEYIGDIRTVDSYFSIYHYVM |
| OJNQ01.1 | VIFMAKKNKMKPRELREAQKKARQLKAAEINNNAAPAIAAMPAAQVIAPAAEKKKSSVKAAGMKS |
| SEQ ID NO: | ILVSENKMYITSFGKGNSAVLEYEVDKVDNNNYNKTQLSSKDNSNIELGDVNEVNITFSSKHGFESG |
| 4855 | VEINTSNPTHRSGESSPVRWDMLGLKSELEKRFFGKTFDDNIHIQLIYNILDIEKILAVYVTNIVYALN |
| | NMLGIKGSESYDDFMGYLSARNTYEVFTHPDKSNLSDKVKGNIKKSLSKFNDLLKTKRLGYFGLEEP |
| | KTKDTRVSQAYKKRVYHMLAIVGQIRQSVFHDKSSKLDEDLYSFIDIIDSEYRETLDYLVDERFDSIN |
| | KGFIQGNKVNISLLIDMMKGYEADDIIRLYYDFIVLKSQKNLGFSIKKLREKMLDEYGFRFKDKQYD |
| | SVRSKMYKLMDFLLFCNYYRNDVVAGEALVRKLRFSMTDDEKEWIYADEAEKLWGKFRNDFENIA |
| | DHMNGDVTKELGKADMDFDEKILDSEKKNASDLLYFSKMIYMLTYFLDGKEINDLLTTLISKFDNIK |
| | EFLKIMKSSAVDVECELTAGYKLFNDSQRITNELFIVKNIASMRKPAASAKLTMFRDALTILGIDDKIT |
| OHIB01.1 | MAKKNKMKPRELREAQKKARQLKAAEINNNAAPAIAAMPAAQVIAPAAEKKKSSVKAAGMKSILV |
| SEQ ID NO: | SENKMYITSFGKGNSAVLEYEVDKVDNNNYNKTQLSSKDNSNIELGDVNEVNITFSSKHGFESGVEI |
| 4856 | NTSNPTHRSGESSPVRWDMLGLKSELEKRFFGKTFDDNIHIQLIYNILDIEKILAVYVTNIVYALNNML |
| | GIKGSESYDDFMGYLSARNTYEVFTHPDKSNLSDKVKGNIKKSLSKFNDLLKTKRLGYFGLEEPKTK |
| | DTRVSQAYKKRVYHMLAIVGQIRQSVFHDKSSKLDEDLYSFIDIIDSEYRETLDYLVDERFDSINKGFI |
| | QGNKVNISLLIDMMKGYEADDIIRLYYDFIVLKSQKNLGFSIKKLREKMLDEYGFRFKDKQYDSVRS |
| | KMYKLMDFLLFCNYYRNDVVAGEALVRKLRFSMTDDEKEWIYADEAEKLWGKFRNDFENIADHM |
| | NGDVIKELGKADMDFDEKILDSEKKNASDLLYFSKMIYMLTYFLDGKEINDLLTTLISKFDNIKEFLK |
| | IMKSSAVDVECELTASYKLFNDSQRITNELFIVKNIASMRKPAASAKLTMFRDALTILGIDDKITDDRI |
| | SEILKLKEKGKGIHGLRNFITNNVIESSRFVYLIKYANAQKIREVAENEKVVMFVLGGIPDTQIERYYK |
| | SCVEFPDMNSSLEVKRSELARMIKNISFDDFKNVKQQAKGRENVAKERAKAVIGLYLTVMYLLVKN |
| | LVNVNARYVIAIHCLERDFGLYKEIIPELASKNLKNDYRILSQTLCELCDDRDESPNLFLKKNKRLRK |
| | CVISIMQTAA |
| OGFI01.1 | MAKKNKMKPRELREAQKKARQLKAAEINNNAAPAIAAMPAAEVIAPAAEKKKSSVKAAGMKSILV |
| SEQ ID NO: | SKNKMYITSFGKGNSAVLEYEVDKVDNNNYNKTQLSSKDNSNIELGDVNEVNITFSSKRGNESGVEI |
| 4857 | NTSNPTHRSGESSPVRWDMLGLKSELEKRFFGKTFDDNIHIQLIYNILDIEKILAVYVTNIVYALNNML |
| | GIKDSESYDDFMGYLSAKNTYEVFTHPDKSNLSDKVKGNIKKSFSTFNDLLKTKRLGYFGLEEPKTK |
| | DTRVSQAYKKRVYHMLAIVGQIRQSVFHDKSSKLDEDLYSFIDIIDSEYRETLDYLVDERFDSINKGFI |
| | QGNKVNISLLIDMMKGYEADDIIRLYYDFIVLKSKKNLGFSIKKLREKMLDEYGFRFKDKQYDSVRS |
| | KMYKLMDFLLFCNYYRNDVIAGEALVRKLRFSMTDDEKEGIYADEAAKLWGKFRNDFENIADHMN |
| | GDVIKELGKADMDFDEKILDSEKKNASNLLYFSKMIYMLTYFLDGKEINDLLTTLISKFDNIKEFLKI |
| | MKSSAVDVECELTAGYKLFNDSQRITNELFIVKNIASMRKPAASAKLTMFRDALTILGIDDNITDDRI |
| | SEILKLKEKGKGIHGLRNFVTNNVIESSRFVYLIKYANAQKIREVAKNEKVVMFVLGGIPDTQIERYY |
| | KSCVEVPDVNSSLEAKRSELARMIKNISFDDFKNVKQ |
| ODWP01.1 | VIFMAKKNKMKPRELREAQKKARQLKAAEINNNAVPAIAAMPAAEVIAPAAEKKKSSVKAAGMKS |
| SEQ ID NO: | ILVSENKMYITSFGKGNSAVLEYEVDKVDNNDYNQTQLSSKDNSNIELGDVNEVNITFSSKHGFESG |
| 4858 | VEINTSNPTHRSGESSPVRWDMLGLKSELEKRFFGKTFDDNIHIQLIYNILDIEKILAVYVTNIVYALN |
| | NMLGIKDSESYDDFMGYLSARNTYEVFTHPDKSNLSDKVKGNIKKSFSTFNDLLKTKRLGYFGLEEP |
| | KTKDTRVSQAYKKRVYHMLAIVGQIRQSVFHDKSSKLHEDLYSFIDIIDSEYRETLDYLVDERFDSIN |
| | KGFIQGNKVNISLLIDMMKGYEADDIIRLYYDFIVLKSQKNLGFSIKKLREKMLDEYGFRFKDKQYD |
| | SVRSKMYKLMDFLLFCNYYRNDIAAGESLVRKLRFSMTDDEKEGIYADEAAKLWGKFRNDFENIAD |
| | HMNGDAIKELGKADMDFDEKILDIEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEVKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNRRLRK |
| | CVEVDINNADSSMTRKYRNCIAHLTVVRELKEYIGDIRTVDSYFSIYHYVMQRCITKRENDTKQEEKI |
| | KYEDDLLKNHG |
| OHCR01.1 | VIFMAKKNKMKPRELREAQKKARQLKAAEINNNAVPAIAAMPAAEVIAPAAEKKKSSVKAAGMKS |
| SEQ ID NO: | ILVSENKMYITSFGKGNSAVLEYEVDNNDYSKTQLSSKDNSNIELGDVNEVNITFSSKHGFESGVEIN |
| 4859 | TSNPTHRSGESSPVRGDMLGLKSELEKRFFGKTFDDNIHIQLIYNILDIEKILAVYVTNIVYALNNMLG |
| | VKGSESYDDFMGYLSAQNTYYIFTHPDKSNLSDKVKGNIKKSLSKFNDLLKTKRLGYFGLEEPKTKD |
| | KRVSEAYKKRVYHMLAIVGQIRQSVFHDKSSKLHEDLYSFIDIIDSEYRETLDYLVDERFDSINKGFIQ |
| | GNKVNISLLIDMMKGYEADDIIRLYYDFIVLKSQKNLGFSIKKLREKMLDEYGFRFKDKQYDSVRSK |
| | MYKLMDFLLFCNYYRNDVVAGEALVRKLRFSMTDDEKEGIYADEAEKLWGKFRNDFENIADHMN |
| | GDVIKELGKADMDFDEKILDSEKKNASDLLYFSKMIYMLTYFLDGKEINDLLTTLISKFDNIKEFLKI |
| | MKSSAVDVECELTAGYKLFNDSQRITNELFIVKNIASMRKPAASAKLTMFRDALTILGIDDKITDDRI |
| | SEILK |
| OKTT01.1 | VIFMAKKNKMKPRELREAQKKARQLKAAEINNNAVPAIAAMPAAEVIAPAAEKKKSSVKAAGMKS |
| SEQ ID NO: | ILVSENKMYITSFGKGNSAVLEYEVDNNDYSKTQLSSKDNSNIELGDVNEVNITFSSKHGFESGVEIN |
| 4860 | TSNPTHRSGESSPVRGDMLGLKSELEKRFFGKTFDDNIHIQLIYNILDIEKILAVYVTNIVYALNNMLG |
| | VKGSESYDDFMGYLSAQNTYYIFTHPDKSNLSDKVKGNIKKSLSKFNDLLKTKRLGYFGLEEPKTKD |
| | KRVSEAYKKRVYHMLAIVGQIRQSVFHDKSSKLHEDLYSFIDIIDSEYRETLDYLVDERFDSINKGFIQ |
| | GNKVNISLLIDMMKGYEADDIIRLYYDFIVLKSQKNLGFSIKKLREKMLDEYGFRFKDKQYDSVRSK |
| | MYKLMDFLLFCNYYRNDVVAGEALVRKLRFSMTDDEKEGIYADEAEKLWGKFRNDFENIADHMN |
| | GDVIKELGKADMDFDEKILDSEKKNASDLLYFSKMIYMLTYFLDGKEINDLLTTLISKFDNIKEFLKI |
| | MKSSAVDVECELTAGYKLFNDSQRITNELFIVKN |
| OJRT01.1 | VLSGIFVNAFSSKHGFESGVEINTSNPTHRSGESSSVRGDMLGLKSELEKRFFGKTFDDNIHIQLIYNIL |
| SEQ ID NO: | DIEKILAVYVTNIVYALNNMLGVKGSESYDDFMGYLSAQNTYYIFTHPDKSNLSDKVKGNIKKSLSK |
| 4861 | FNDLLKTKRLGYFGLEEPKTKDKRVSEAYKKRVYHMLAIVGQIRQSVFHDKSNELDEYLYSFIDIIDS |
| | EYRDTLDYLVDERFDSINKGFVQGNKVNISLLIDMMKGYEADDIIRLYYDFIVLKSQKNLGFSIKKLR |
| | EKMLDEYGFRFKDKQYDSVRSKMYKLMDFLLFCNYYRNDVVAGEALVRKLRFSMTDDEKEGIYA |
| | DEAEKLWGKFRNDFENIADHMNGDVIKELGKADMDFDEKILDSEKKNASDLLYFSKMIYMLTYFLD |
| | GKEINDLLTTLISKFDNIKEFLKIMKSSAVDVECELTAGYKLFNDSQRITNELFIVKNIASMRKPAASA |
| | KLTMFRDALTILGIDDKITDDRISEILKLKEKGKGIHGLRNFITNNVIESSRFVYLIKYANAQKIREVAK |
| | N |
| OGOJ01.1 | VLSGIFVNAFSSKHGFESGVEINTSNPTHRSGESSSVRGDMLGLKSELEKRFFGKTFDDNIHIQLIYNIL |
| SEQ ID NO: | DIEKILAVYVTNIVYALNNMLGVKGSESYDDFMGYLSAQNTYYIFTHPDKSNLSDKVKGNIKKSLSK |
| 4862 | FNDLLKTKRLGYFGLEEPKTKDKRVSEAYKKRVYHMLAIVGQIRQSVFHDKSNELDEYLYSFIDIIDS |
| | EYRDTLDYLVDERFDSINKGFVQGNKVNISLLIDMMKGYEADDIIRLYYDFIVLKSQKNLGFSIKKLR |
| | EKMLDEYGFRFKDKQYDSVRSKMYKLMDFLLFCNYYRNDVVAGEALVRKLRFSMTDDEKEGIYA |
| | DEAEKLWGKFRNDFENIADHMNGDVIKELGKADMDFDEKILDSEKKNASDLLYFSKMIYMLTYFLD |
| | GKEINDLLTTLISKFDNIKEFLKIMKSSAVDVEC |
| OLVY01.1 | MAKKNKMKPRELREAQKKARQLKAAEINNNAVPAIAAMPAAEVIAPAAEKKKSSVKAAGMKSILV |
| SEQ ID NO: | SENKMYITSFGKGNSAVLEYEVDNNDYNQTQLSSDGSSNIELRGVNEVNITFSSKHGFESGVEINTSN |
| 4863 | PTHRSGESSPVRGDMLGLKSELEKRFFGKTFDDNIHIQLIYNILDIEKILAVYVTNIVYALNNMLGVK |
| | GSESYDDFMGYLSAQNTYYIFTHPDKSNLSDKVKGNIKKSLSKFNDLLKTKRLGYFGLEEPKTKDKR |
| | VSEAYKKRVYHMLAIVGQIRQSVFHDKSNELDEYLYSFIDIIDSEYRDTLDYLVDERFDSINKGFVQG |
| | NKVNISLLIDMMKGYEADDIIRLYYDFIVLKSQKNLGFSIKKLREKMLDEYGFRFKDKQYDSVRSKM |
| | YKLMDFLLFCNYYRNDVVAGEALVRKLRFSMTDDEKEGIYADEAEKLWGKFRNDFENIADHMNG |
| | DVIKELGKADMDFDEKILDSEKKNASDLLYFSKMIYMLTYFLDGKEINDLLTTLISKFDNIKEFLKIM |
| | KSSAVDVECELTAGYKLFNDSQRITNELFIVKNIASMRKPAASAKLTMFRDALTILGIDDKITDDRISE |
| | ILKLKEKGKGIHGLRNFITNNVIESSRFVYLIKYANAQKIREVAKNEKWMFVLGGIPDTQIERYYKS |
| | CVEFPDMNSSLEAKRSELARMIKNISFDDFKNVKQQAKGRENVAKERAKAVIGLYLTVMYLLVKNL |
| | VNVNARYVIAIHCLERDFGLYKEIIPELASKNLKNDYRILSQTLCELCDDRDELPNLFLKKNERLRKC |
| | VEVDINNADNFVSATEGNLFDFFAHFSVTY |
| ULPU01.1 | VIFMAKKNKMKPRELREAQKKARQLKAAEINNNAVPAIAAMPAAEVIAPAAEKKKSSVKAAGMKS |
| SEQ ID NO: | ILVSENKMYITSFGKGNSAVLEYEVDNNDYNQTQLSSDGSSNIELRGVNEVNITFSSKHGFESGVEIN |
| 4864 | TSNPTHRSGESSPVRGDMLGLKSELEKRFFGKTFDDNIHIQLIYNILDIEKILAVYVTNIVYALNNMLG |
| | VKGSESYDDFMGYLSAQNTYYIFTHPDKSNLSDKVKGNIKKSLSKFNDLLKTKRLGYFGLEEPKTKD |
| | KRVSEAYKKRVYHMLAIVGQIRQSVFHDKSNELDEYLYSFIDIIDSEYRDTLDYLVDERFDSINKGFV |
| | QGNKVNISLLIDMMKGYEADDIIRLYYDFIVLKSQKNLGFSIKKLREKMLDEYGFRFKDKQYDSVRS |
| | KMYKLMDFLLFCNYYRNDVVAGEALVRKLRFSMTDDEKEGIYADEAEKLWGKFRNDFENIADHM |
| | NGDVIKELGKADMDFDEKILDSEKKNASDLLYFSKMIYMLTYFLDGKEINDLLTTLISKFDNIKEFLK |
| | IM |
| UZRW01.1 | VIFMAKKNKMKPRELREAQKKARQLKAAEINNNAAPAIAAMPAVEVIAPAAEKKKSSVKAAGMKS |
| SEQ ID NO: | ILVSENKMYITSFGKGNSAVLEYEVDKVDNNDYNKTQLSSKNSSNIELRGVNEVNITFSSKHGFESGV |
| 4865 | EINTSNPTHRSGESSPVRWDMLGLKSELEKRFFGKTFDDNIHIQLIYNILDIEKILAVYVTNIVYALNN |
| | MLGIKDSESYDDFMGYLSAKNTYEVFTHPDKSDLSDKVKGNIKKSFSTFNDLLKTKRLGYFGLEEPK |
| | TKDTRVSQAYKKRVYHMLAIVGQIRQSVFHDKSSKLDEDLYSFIDIIDPEYRETLDYLVDERFDSINK |
| | GFIQGNKVNISLLIDMMKGYEADDIIRLYYDFIVLKSQKNLGFSIKKLREKMLDEYGFRFKDKQYDS |
| OZJV01.1 | VIFMAKKNKMKPRELREAQKKARQLKAAEINNNAAPAIAAMSAAEVIAPAAEKKKSSVKAAGMKS |
| SEQ ID NO: | ILVSENKMYITSFGKGNSAVLEYEVDNNDYNKTQLSSKDNSNIELGDVNEVNITFSSKHGFGSGVEIN |
| 4866 | TSNPTHRSGESSPVRWDMLGLKSELEKRFFGKTFDDNIHIQLIYNILDIEKILAVYVTNIVYALNNMLG |
| | VKGSESYDDFMGYLSAKNTYEVFTHPDKSNLSDKVKGNIKKSFSTFNDLLKTKRLGYFGLEEPKTK |
| | DTRVSQAYKKRVYHMLAIVGQIRQSVFHDKSSKLHEDLYSFIDIIDSEYRETLDYLVEERLKSINKDFI |
| | EGNKVNISLLIDMMKGYEADDIIRLYYDFIVLKSQKNLGFSIKKLREKMLDEYGFRFKDKQYDPVRS |
| | KMYKLMDFLLFCNYYRNDVVTGEALVRKLRFSMTDDEKEGIYADEASKLWGKFRNDFENIADHM |
| | NGDVIKELGKADMDFDEKILDSEKKNASDLLYFSKMIYMLTYFLDGKEINDLLTTLISKFDNIKEFLK |
| | IMKSSAVDVECELTAGYKLFNDSQRITNELFIVKNIASMRKPAASAKLTMFRDALTILGIDDNITDDRI |
| | SEILKLKEKGKGIHGLRNFITNNVIESSRFVYL |
| ULOZ01.1 | VIFMAKKNKMKPRELREAQKKARQLKAAEINNNAAPAIAAMSAAEVIAPAAEKKKSSVKAAGMKS |
| SEQ ID NO: | ILVSENKMYITSFGKGNSAVLEYEVDNNDYNKTQLSSKDNSNIELGDVNEVNITFSSKHGFGSGVEIN |
| 4867 | TSNPTHRSGESSPVRWDMLGLKSELEKRFFGKTFDDNIHIQLIYNILDIEKILAVYVTNIVYALNNMLG |
| | VKGSESYDDFMGYLSAKNTYEVFTHPDKSNLSDKVKGNIKKSFSTFNDLLKTKRLGYFGLEEPKTK |
| | DTRVSQAYKKRVYHMLAIVGQIRQSVFHDKSSKLHEDLYSFIDIIDSEYRETLDYLVEERLKSINKDFI |
| | EGNKVNISLLIDMMKGYEADDIIRLYYDFIVLKSQKNLGFSIKKLREKMLDEYGFRFKDKQYDPVRS |
| | KMYKLMDFLLFCNYYRNDVVTGEALVRKLRFSMTDDEKEGIYADEASKLWGKFRNDFENIADHM |
| | NGDVIKELGKADMDFDEKILDSEKKNASDLLYFSKMIYMLTYFLDGKEINDLLTTLISKFDNIKEFLK |
| | IMKSSAVDVE |
| ULQA01.1 | VIFMAKKNKMKPRELREAQKKARQLKAAEINNNAAPAIAAMSAAEVIAPAAEKKKSSVKAAGMKS |
| SEQ ID NO: | ILVSENKMYITSFGKGNSAVLEYEVDNNDYNKTQLSSKDNSNIELGDVNEVNITFSSKHGFGSGVEIN |
| 4868 | TSNPTHRSGESSPVRWDMLGLKSELEKRFFGKTFDDNIHIQLIYNILDIEKILAVYVTNIVYALNNMLG |
| | VKGSESYDDFMGYLSAKNTYEVFTHPDKSNLSDKVKGNIKKSFSTFNDLLKTKRLGYFGLEEPKTK |
| | DTRVSQAYKKRVYHMLAIVGQIRQSVFHDKSSKLHEDLYSFIDIIDSEYRETLDYLVEERLKSINKDFI |
| | EGNKVNISLLIDMMKGYEADDIIRLYYDFIVLKSQKNLGFSIKKLREKMLDEYGFRFKDKQYDPVRS |
| | KMYKLMDFLLFCNYYRNDVVTGEALVRKLRFSMTDDEKEGIYADEAPKLWGKFRNDFENIADHM |
| | NGDVIKELGKADMDFDEKILDSEKKNASDLLYFSKMIYMLTYFLDGKEINDLLTTLISKFDNIKEFLK |
| | IMK |
| OPMM01.1 | MAKKNKMKPRELREAQKKARQLKAAEINNNAAPAIAAMSAAEVIAPAAEKKKSSVKAAGMKSILV |
| SEQ ID NO: | SENKMYITSFGKGNSAVLEYEVDNNDYNKTQLSSKDNSNIELGDVNEVNITFSSKHGFGSGVEINTS |
| 4869 | NPTHRSGESSPVRWDMLGLKSELEKRFFGKTFDDNIHIQLIYNILDIEKILAVYVTNIVYALNNMLGV |
| | KGSESYDDFMGYLSAKNTYEVFTHPDKSNLSDKVKGNIKKSFSTFNDLLKTKRLGYFGLEEPKTKDT |
| | RVSQAYKKRVYHMLAIVGQIRQSVFHDKSSKLHEDLYSFIDIIDSEYRETLDYLVEERLKSINKDFIEG |
| | NKVNISLLIDMMKGYEADDIIRLYYDFIVLKSQKNLGFSIKKLREKMLDEYGFRFKDKQYDPVRSKM |
| | YKLMDFLLFCNYYRNDVVTGEALVRKLRFSMTDDEKEGIYADEASKLWGKFRNDFENIADHMNGD |
| | VIKELGKADMDFDEKILDSEKKNASDLLYFSKMIYMLTYFLDGKEINDLLTTLISKFDNIKEFLKIMK |
| | NASKERLMLIKLK |
| UXYB01.1 | VIFMAKKNKMKPRELREAQKKARQLKAVEINNNAVPEIAAMPAAEVIAPVAEKKKSSVKAAGMKSI |
| SEQ ID NO: | LVSENKMYITSFGKGNSAVLEYEVDNNDYNKTQLSSKNSSNIELRGVNEVNITFSSKHGFESGVEINT |
| 4870 | SNPTHRSGESSPVRWDMLGLKSELEKRFFGKTFDDNIHIQLIYNILDIEKILAVYVTNIVYALNNMLGI |
| | KGSESYDDFMGYLSARNTYEVFTHPDKSSLSDDKKANVRKSLSKFNVLLKTKRLGYFGLEEPKTKD |
| | TRVSQAYKKRVYHMLAIVGQIRQSVFHDKSSKLDEDLYSFIDIIDSEYRETLDYLVDERFDSINKGFIQ |
| | GNKVNISLLIDMMKGYEADDIIRLYYDFIVLKSQKNLGFSIKKLREKMLEEYGYRFKDKQYDSVRSK |
| | MYKLMDFLLFCNYYRNDVVAGEALVRKLRFSMTDDEKEGIYADEAEKLWGKFRNDFENIADHMN |
| | GDVIKELGKADMDFDEKILDSEKKNASDLLYFSKMIYMLTYFLDGKEINDLLTTLISKFDNIKEFLKI |
| | MKSSAVDVECELTAGYKLFNDSQRITNELFIVKNIASMRKPAASAKLTMFRDALTILGIDDKITDDRI |
| | SEILKLKEKGKGIHGLRNFITNNVIESSRFVYLIKYANAQKIREVAENEKVVMFVLGGIPDTQIERYYK |
| | SCVEFPDMNSSLEAKRSELARMIKNISFDNFKNVKQQAKGRENVAKERAKAVIGLYLTVMYLLVKN |
| | LVNVNARYVIAIHCLERDFGLYKEIIPELASKNLKNDYRILSQTLCELCDDRDES |
| OPKZ01.1 | VIFMAKKNKMKPRELREAQKKARQLKAVEINNNAVPEIAAMPAAEVIAPVAEKKKSSVKAAGMKSI |
| SEQ ID NO: | LVSENKMYITSFGKGNSAVLEYEVDNNDYNKTQLSSKNSSNIELRGVNEVNITFSSKHGFESGVEINT |
| 4871 | SNPTHRSGESSPVRWDMLGLKSELEKRFFGKTFDDNIHIQLIYNILDIEKILAVYVTNIVYALNNMLGI |
| | KGSESYDDFMGYLSARNTYEVFTHPDKSSLSDDKKANVRKSLSKFNVLLKTKRLGYFGLEEPKTKD |
| | TRVSQAYKKRVYHMLAIVGQIRQSVFHDKSSKLDEDLYSFIDIIDSEYRETLDYLVDERFDSINKGFIQ |
| | GNKVNISLLIDMMKGYEADDIIRLYYDFIVLKSQKNLGFSIKKLREKMLEEYGYRFKDKQYDSVRSK |
| | MYKLMDFLLFCNYYRNDVVAGEALVRKLRFSMTDDEKEGIYADEAEKLWGKFRNDFENIADHMN |
| | GDVIKELGKADMDFDEKILDSEKKNASDLLYFSKMIYMLTYFLDGKEINDLLTTLISKFDNIKEFLKI |
| | MKSSAVDVECELTAGYKLFNDSQRITNELFIVKNIASMRKPAASAKLTMFRDALTILGIDDKITDDRI |
| | SGILKF |
| OPXF01.1 | MAKKNKMKPRELREAQKKARQLKAVEINNNAVPEIAAMPAAEVIAPVAEKKKSSVKAAGMKSILV |
| SEQ ID NO: | SENKMYITSFGKGNSAVLEYEVDNNDYNKTQLSSKNSSNIELRGVNEVNITFSSKHGFESGVEINTSN |
| 4872 | PTHRSGESSPVRWDMLGLKSELEKRFFGKTFDDNIHIQLIYNILDIEKILAVYVTNIVYALNNMLGIKG |
| | SESYDDFMGYLSARNTYEVFTHPDKSSLSDDKKANVRKSLSKFNVLLKTKRLGYFGLEEPKTKDTR |
| | VSQAYKKRVYHMLAIVGQIRQSVFHDKSSKLHEDLYSFIDIIDSEYRETLDYLVDERFDSINKGFIQG |
| | NKVNISLLIDMMKDDYEADDIIRLYYDFIVLKSQKNLGFSIKKLREKMLDEYGFRFKDKQYDSVRSK |
| | MYKLMDFLLFCNYYRNDVIAGEALVRKLRFSMTDDEKEGIYADEAAKLWGKFRNDFENIADHMNG |
| | DVIKELGKADMDFDEKILDSEKKNASDLLYFSKMIYMLTYFLDGKEINDLLTTLISKFDNIKEFLKIM |
| | KSSAVDVECELTAGYKLFNDSQRITNELFIVKNIASMRKPAASAKLTMFRDALTILGIDDNITDDRISE |
| | ILKLKEKGKGIHGLRNFITNNVIESSRFVYLIKYANAQKIREVAKNEKVVMFVLGGIPDTQIERYYKS |
| | CVEFPDMNSSLEAKRSELARMIKNISFDDFKNVKQQAKGRENVAKERAKAVIGLYLTVMYLLVKNL |
| | VNVNARYVIAIHCLERDFGLYKEIIPELASKNLKNDYRILSQTLCELCDNGDESPNLFLKKNKLPTRSL |
| | PGY |
| OGYM01.1 | VIFMAKKNKMKPRELREAQKKARQLKAAEINNNAAPAIAAMPAAEVIAPVAGKKKSSVKAAGMKS |
| SEQ ID NO: | ILVSENKMYITSFGKGNSAVLEYEVDKVDNNDYNQTQLSSKGSSNIELCGVNEVNITFSSKHGFESGV |
| 4873 | EINTSNPTHRSGESSPVRWDMLGLKSELEKRFFGKTFDDNIHIQLIYNILDIEKILAVYVTNIVYALNN |
| | MLGIKKSESYDDFMGYLSARNTYEVFTHPDKSNLSDKAKGNIKKSFSTFNDLLKTKRLGYFGLEEPK |
| | TKDTRVSQAYKKRVYHMLAIVGQIRQSVFHDKSSKLDEDLYSFIDIIDSEYRETLDYLVEERLKSINK |
| | DFIEGNKVNISLLIDMMKGFEADDIIRLYYDFIVLKSQKNLGFSIKKLREKMLEEYGYRFKDKQYDSV |
| | CSKMYKLMDFLLFCNYYRNDVVAGEALVRKLRFSMTDDEKEGIYADEAAKLWGKFRNDFENIADH |
| | MNGDVTKELGKADMDFDEKILDSEKKNASDLLYFSKMIYMLTYFLDGKEINDLLTTLISKFDNIKEFL |
| | KIMKSSAVDVECELTAGYKLFNDSQRITNELFIVKNIASMRKPAASAKLTMFRDALTILGIDDKITDD |
| | RISEILKLKEKGKGIHGLRNFITNNVIESSRFVYLIKYANAQKIREVAKNEKVVMFVLGGIPDTQIERY |
| | YKSCVEFPDMNSSLEVKRSELARMIKNISFDDFKNVKQQAKGRENVAKERAKAVIGLYLTVMYLLV |
| | KNLVNVNARYVIAIHCLERDFGLYKEIIPELASKNLKNDYRILSQTLCELCDDRDKSSNLFLKKNKRL |
| | RKCVEVDINNADSSMTRKYRNCIAH |
| UEOI01.1 | VIFMAKKNKMKPRELREAQKKARQLKAAEINNNAVPAIAAMPAAEAAAPAAEKKKSSVKAAGMK |
| SEQ ID NO: | SILVSENKMYITSFGKGNSAVLEYEVDKVDDNDYNKTQLSSKGSSNIELHGVNEVNITFSSKHGFESG |
| 4874 | VEINTSNPTHRSGESSPVRGDMLGLKSELEKRFFGKTFDDNIHIQLIYNILDIEKILAVYVTNIVYALNN |
| | MLGIKGSESYDDFMGYLSARNTYEVFTNPNGSTLSDDKKENIRKSLSKFNALLKTKRLGYFGLEEPK |
| | TKDTRVSQAYKKRVYHMLAIVGQIRQSVFHDKSSKLDEDLYSFIDIIDSEYRETLDYLVDERFDSINK |
| | GFIQGNKVNISLLIDMMKDDYEADDIIRLYYDFIVLKSQKNLGFSIKKLREKMLDEYGFRFKDKQYD |
| | SVRSKMYKLMDFLLFCNYYRNDVVAGEALVRKLRFSMTDDEKEGTYADEAEKLWGKFRNDFENIA |
| | DHMNGDAIKELGKADMDFDEKILDSEKKNASDLLYFSKMIYMLTYFLDGKEINDLLTTLISKFDNIK |
| | EFLKIMKSSAVDVECELTAGYKLFNDSQRITNELFIVKNIASMRKPAASAKLTMFRDALTILGIDDNIT |
| | DDRISEILKLKEKGK |
| OGZV01.1 | MAKKNKMKPRELREAQKKARQLKAAEINNNAAPAIAAMPAAEVIAPAAEKKKSSVKAAGMKSILV |
| SEQ ID NO: | SENKMYITSFGKGNSAVLEYEVDNNDYNKTQLSSKDNSNIELGNVNEVNITFSSRRGFESGVEINTSN |
| 4875 | PTHRSGESSSVRGDMLGLKSELEKRFFGKTFDDNIHIQLIYNILDIEKILAVYVTNIVYALNNMLGVK |
| | GSESYDDFMGYLSAQNTYYIFTHPDKSNLSDKVKGNIKKSLSKFNDLLKTKRLGYFGLEEPKTKDTR |
| | VSQAYKKRVYHMLAIVGQIRQCVFHDKSGAKRFDIYSFINNIDPEYRETLDYLVDERFDSINKGFIQG |
| | NKVNISLLIDMMKDYEADDIIRLYYDFIVLKSQKNLGFSIKKLREKMLDEYGFRFKDKQYDSVRSKM |
| | YKLMDFLLFCNYYRNDVVAGETLVRKLRFSMTDDEKEGIYADEAAKLWGKFRNDFENIADHMNG |
| | DVIKELGKADMDFDEKILDSEKKNASDLLYFSKMIYMLTYFLDGKEINDLLTTLISKFDNIKEFLKIM |
| | KSSAVDVECELTEGYKLFNDSQRITNELFIVKNIASMRKPAASAKLTMFRDALTILGIDDKITDDRISEI |
| | LKLKEKGKGIHGLRNFITNNVIESSRFVYLIKYANAQKIREVAKNEKWMFVLGGIPDTQIERYYKSC |
| | VEFPDMNSSLEAKRSELARMIKNIRFQKCETAGKGQRKRG |
| ULPA01.1 | MAKKNKMKPRELREAQKKARQLKAAEINNNAAPAIAAMPAAEVIAPVAEKKKSSVKAAGMKSILV |
| SEQ ID NO: | SENKMYITSFGKGNSAVLEYEVDNNDYNKTQLSSKDNSNIELGDVNEVNITFSSKHGFGSGVEINTS |
| 4876 | NPTHRSGESSPVRGDMLGLKSELEKRFFGKTFDDNIHIQLIYNILDIEKILAVYVTNIVYALNNMLGIK |
| | DSESYDDFMGYLSARNTYEVFTHPDKSNLSDKVKGNIKKSLSKFNDLLKTKRLGYFGLEEPKTKDTR |
| | ASEAYKKRVYHMLAIVGQIRQCVFHDKSGAKRFDLYSFINNIDPEYRDTLDYLVEERLKSINKDFIEG |
| | NKVNISLLIDMMKGYEADDIIRLYYDFIVLKSQKNLGFSIKKLREKMLEEYGFRFKDKQYDSVRSKM |
| | YKLMDFLLFCNYYRNDVAAGEALVRKLRFSMTDDEKEGIYADEAAKLWGKFRNDFENIADHMNG |
| | DVIKELGKADMDFDEKILDSEKKNASDLLYFSKMIYMLTYFLDGKEINDLLTTLISKFDNIKEFLKIM |
| | KSSAVDVECELTAGYKLFNDSQRITNELFIVKNIASM |
| UMGR01.1 | MAKKNKMKPRELREAQKKARQLKAAEINNNAAPAIAAMPAAEVIAPVAEKKKSSVKAAGMKSILV |
| SEQ ID NO: | SENKMYITSFGKGNSAVLEYEVDNNDYNKTQLSSKDNSNIELGDVNEVNITFSSKHGFGSGVEINTS |
| 4877 | NPTHRSGESSPVRGDMLGLKSELEKRFFGKTFDDNIHIQLIYNILDIEKILAVYVTNIVYALNNMLGIK |
| | DSESYDDFMGYLSARNTYEVFTHPDKSNLSDKVKGNIKKSLSKFNDLLKTKRLGYFGLEEPKTKDTR |
| | ASEAYKKRVYHMLAIVGQIRQCVFHDKSGAKRFDLYSFINNIDPEYRDTLDYLVEERLKSINKDFIEG |
| | NKVNISLLIDMMKGYEADDIIRLYYDFIVLKSQKNLGFSIKKLREKMLEEYGFRFKDKQYDSVRSKM |
| | YKLMDFLLFCNYYRNDVAAGEALVRKLRFSMTDDEKEGIYADEAAKLWGKFRNDFENIADHMNG |
| | DVIKELGKADMDFDEKILDSEKKNASDLLYFSKMIYMLTYFLDGKEINDLLTTLISKFDNIKEFLKIM |
| | KSSAVDVECELTAGYKLFNDSQRITNELFIVKNIASMRKPAASAKLTMFRDALTILGIDDNITDDRISE |
| | ILKLKEKGKGIHGLRNFITNNVTESSRFVYLIKYANAQK |
| OMER01.1 | MAKKNKMKPRELREAQKKARQLKAAEINNNAAPAIAAMPAAEVIAPVAEKKKSSVKAAGMKSILV |
| SEQ ID NO: | SENKMYITSFGKGNSAVLEYEVDNNDYNKTQLSSKDNSNIELGDVNEVNITFSSKHGFGSGVEINTS |
| 4878 | NPTHRSGESSPVRGDMLGLKSELEKRFFGKTFDDNIHIQLIYNILDIEKILAVYVTNIVYALNNMLGIK |
| | DSESYDDFMGYLSARNTYEVFTHPDKSNLSDKVKGNIKKSLSKFNDLLKTKRLGYFGLEEPKTKDTR |
| | ASEAYKKRVYHMLAIVGQIRQCVFHDKSGAKRFDLYSFIKNIDPEYRDTLDYLVEERLKSINKDFIEG |
| | NKVNISLLIDMMKGYEADDIIRLYYDFIVLKSQKNLGFSIKKLREKMLEEYGFRFKDKQYDSVRSKM |
| | YKLMDFLLFCNYYRNDVAAGEALVRKLRFSMTDDEKEGIYADEAAKLWGKFRNDFENIADHMNG |
| | DVIKELGKADMDFDEKILDSEKKNASDLLYFSKMIYMLTYFLDGKEINDLLTTLISKFDNIKEFLKIM |
| | KSSAVDVECELTAGYKLFNDSQRITNELFIVKNIASMRKPAASAKLTMFRDALTILGIDDNIT |
| UMCG01.1 | VIFMAKKNKMKPRELREAQKKARQLKAAEIKNNAVPAIAAMPAAEAAAPAVEKKKSSVKAAGMK |
| SEQ ID NO: | SILVSENKMYITSFGKGNSAVLEYEVDKVDNNNYNKTQLSSKDNSNIELGDVNEVNITFSSKRGNES |
| 4879 | GVEINTSNPTHRSGESSPVRWDMLGLKSELEKRFFGKTFDDNIHIQLIYNILDIEKILAVYVTNIVYAL |
| | NNMLGIKGSESYDDFMGYLSARNTYEVFTHPDKSNLSDKVKGNIKKSLSKFNDLLKTKRLGYFGLE |
| | EPKTKDTRVSQAYKKRVYHMLAIVGQIRQCVFHDKSGAKRFDLYSFINNIDPEYRDTLDYLVEERLK |
| | SINKDFIEGNKVNISLLIDMMKGYEADDIIRLYYDFIVLKSQKNLGFSIKKLREKMLEEYGYRFKDKQ |
| | YDSVRSKMYKLMDFLLFCNYYRNDVVAGEALVRKLRFSMTDDEKEGIYADEASKLWGKFRNDFE |
| | NIADHMNGDVIKELGKADMDFDEKILDSEKKNASDLLYFSKMIYMLTYFLDGKEINDLLTTLISKFD |
| | NIKEFLKIMKSSAVDVECALTAGYKLFNDSQRITNELFIVKNIASMRKPAASAKLTMFRDALTILGID |
| | DKITDDRISEILKLKEKGKGIHGLRNFITNNVTESSRFVYLIKYANAQKIREVAKNEKWMFVLGGIPD |
| | TQIERYYKSCVEFPDMNSSLEAKRSELARMIKNIRFDDFKNVKQQAKGRENVAKERAKAVIGLYLT |
| | VMYLLVKNLVNVNARYVIAIHCLERDFGLYKEIIPELASKNLKNDYRILSQTLCELCDDRDESPNLFL |
| | KKNKRLRKCVEVDINNADSSMTRKYRNCIAHLTVVRELKEYIGDIRTVDSYFSIYHYVMQRCITKRE |
| | DDTKQEEKIKYE |
| UMCJ01.1 | VIFMAKKNKMKPRELREAQKKARQLKAAEIKNNAVPAIAAMPAAEAAAPAVEKKKSSVKAAGMK |
| SEQ ID NO: | SILVSENKMYITSFGKGNSAVLEYEVDKVDNNNYNKTQLSSKDNSNIELGDVNEVNITFSSKRGNES |
| 4880 | GVEINTSNPTHRSGESSPVRWDMLGLKSELEKRFFGKTFDDNIHIQLIYNILDIEKILAVYVTNIVYAL |
| | NNMLGIKGSESYDDFMGYLSARNTYEVFTHPDKSNLSDKVKGNIKKSLSKFNDLLKTKRLGYFGLE |
| | EPKTKDTRVSQAYKKRVYHMLAIVGQIRQCVFHDKSGAKRFDLYSFINNIDPEYRDTLDYLVEERLK |
| | SINKDFIEGNKVNISLLIDMMKGYEADDIIRLYYDFIVLKSQKNLGFSIKKLREKMLEEYGYRFKDKQ |
| | YDSVRSKMYKLMDFLLFCNYYRNDVVAGEALVRKLRFSMTDDEKEGIYADEASKLWGKFRNDFE |
| | NIADHMNGDVIKELGKADMDFDEKILDSEKKNASDLLYFSKMIYMLTYFLDGKEINDLLTTLISKFD |
| | NIKEFLKIMKSSAVDVECELTAGYKLFNDSQRITNELFIVKNIASMRKPAASAKLTMFRDALTILGIDD |
| | KITDDRISEILKLKEKGKGIHGLRNFITNNVTESSRFVYLIKYANAQKIREVAKNEKWMFVLGGIPDT |
| | QIERYYKSCVEFPDMNSSLEAKRSELARMIKNIRFDDFKNVKQQAKGRENVAKERAKAVIGLYLTV |
| | MYLLVKNLVNVNARYVIAIHCLERDFGLYKEIIPELASKNLKNDYRILSQTLCELCDDRDESPNLFLK |
| | KNKRLRKCVEVDINNADSSMTRKYRNCIAHLTVVRELKEYIGDIRTVDSYFSIYHYVMQRCITKRED |
| | D |
| OPXM01.1 | VIFMAKKNKMKPRELREAQKKARQLKAAEIKNNAVPAIAAMPAAEAAAPAVEKKKSSVKAAGMK |
| SEQ ID NO: | SILVSENKMYITSFGKGNSAVLEYEVDKVDNNNYNKTQLSSKDNSNIELGDVNEVNITFSSKRGNES |
| 4881 | GVEINTSNPTHRSGESSPVRWDMLGLKSELEKRFFGKTFDDNIHIQLIYNILDIEKILAVYVTNIVYAL |
| | NNMLGIKGSESYDDFMGYLSARNTYEVFTHPDKSNLSDKVKGNIKKSLSKFNDLLKTKRLGYFGLE |
| | EPKTKDTRVSQAYKKRVYHMLAIVGQIRQCVFHDKSGAKRFDLYSFINNIDPEYRDTLDYLVEERLK |
| | SINKDFIEGNKVNISLLIDMMKGYEADDIIRLYYDFIVLKSQKNLGFSIKKLREKMLEEYGYRFKDKQ |
| | YDSVRSKMYKLMDFLLFCNYYRNDVVAGEALVRKLRFSMTDDEKEGIYADEASKLWGKFRNDFE |
| | NIADHMNGDVIKELGKADMDFDEKILDSEKKNASDLLYFSKMIYMLTYFLDGKEINDLLTTLISKFD |
| | NIKEFLKIMKSSAVDVECELTAGYKLFNDSQRITNELFIVKNIASMRKPAASAKLTMFRDALTILGIDD |
| | KITDDRISEILKLKEKGKGIHGLRNFITNNVIESSRFVYLIKYANAQKIREVAKNEKVVMFVLGGIPDT |
| | QIERYYKSCVEFPDMNSSLEAKRSELARMIKNIRFDDFKNVKQQAKGRENVAKERAKAVIGLYLTV |
| | MYLLVKNLVNVNARYVIAIHCLERDFGLYKEIIPELASKNLKNDYRILSQTLCELCDDRDESPNLFLK |
| | KNKRLRKCVEVDINNADSSMTRKYRNCIAHLTVVRELKEYIGDIRTVDSYFSIYHYVMQRCITKRED |
| | DTKQEEKIKYEDDLLKNH |
| CEAG01.1 | MAKKNKMKPRELREAQKKARQLKAAEINNNAAPAIAAMPAAEAIAPAAEKKKSSVKAAGMKSIFV |
| SEQ ID NO: | SENKMYITSFGKGNSAVLEYEVDKVDNNVYNQTQLSSEDSSNIELCGVTKVNITFSSKHGLESGVEIS |
| 4882 | TSNPTHRSGESSPVRWDMLGLKSELEKRFFGKTFDDNIHIQLIYNILDIEKILAVYVTNIVYALNNMLG |
| | IKDSESYDDFMGYLSARNTYKVFTHPDKSNLSDKVKGNIKKSFSTFNDLLKTKRLGYFGLEEPKTKD |
| | TRVSEAYKKRVYHMLAIVGQIRQCVFHDLSEHSEYDLYSFIDNSKKVYRECRETLNYLVDERFDSIN |
| | KGFIQGNKVNISLLIDMMKDDYEADDIIHLYYDFIVLKSQKNLGFSIKKLREKMLDEYGFRFKDKQY |
| | DSVRSKMYKLMDFLLFCNYYRNDVVAGEALVRKLRFSMTDDEKEGIYADEAEKLWGKFRNDFENI |
| | ADHMNGDVTKELGKADMDFDEKIIDSEKKNASDLLYFSKMIYMLTYFLDGKEINDLLTTLISKFDNIK |
| | EFLKIMKSSAVDVE |
| CEAH01.1 | MAKKNKMKPRELREAQKKARQLKAAEINNNAAPAIAAMPAAEAIAPAAEKKKSSVKAAGMKSIFV |
| SEQ ID NO: | SENKMYITSFGKGNSAVLEYEVDKVDNNVYNQTQLSSEDSSNIELCGVTKVNITFSSKHGLESGVEIS |
| 4883 | TSNPTHRSGESSPVRWDMLGLKSELEKRFFGKTFDDNIHIQLIYNILDIEKILAVYVTNIVYALNNMLG |
| | IKDSESYDDFMGYLSARNTYKVFTHPDKSNLSDKVKGNIKKSFSTFNDLLKTKRLGYFGLEEPKTKD |
| | TRVSEAYKKRVYHMLAIVGQIRQCVFHDLSEHSEYDLYSFIDNSKKVYRECRETLNYLVDERFDSIN |
| | KGFIQGNKVNISLLIDMMKDDYEADDIIHLYYDFIVLKSQKNLGFSIKKLREKMLDEYGFRFKDKQY |
| | DSVRSKMYKLMDFLLFCNYYRNDVVAGEALVRKLRFSMTDDEKEGIYADEAEKLWGKFRNDFENI |
| | ADHMNGDVTKELGKADMDFDEKIIDSEKKNASDLLYFSKMIYMLTYFLDGKEINDLLTTLISKFDNIK |
| | EFLKIMKSSAVDVE |
| CEAF01.1 | MAKKNKMKPRELREAQKKARQLKAAEINNNAAPAIAAMPAAEAIAPAAEKKKSSVKAAGMKSIFV |
| SEQ ID NO: | SENKMYITSFGKGNSAVLEYEVDKVDNNVYNQTQLSSEDSSNIELCGVTKVNITFSSKHGLESGVEIS |
| 4884 | TSNPTHRSGESSPVRWDMLGLKSELEKRFFGKTFDDNIHIQLIYNILDIEKILAVYVTNIVYALNNMLG |
| | IKDSESYDDFMGYLSARNTYKVFTHPDKSNLSDKVKGNIKKSFSTFNDLLKTKRLGYFGLEEPKTKD |
| | TRVSEAYKKRVYHMLAIVGQIRQCVFHDLSEHSEYDLYSFIDNSKKVYRECRETLNYLVDERFDSIN |
| | KGFIQGNKVNISLLIDMMKDDYEADDIIHLYYDFIVLKSQKNLGFSIKKLREKMLDEYGFRFKDKQY |
| | DSVRSKMYKLMDFLLFCNYYRNDVVAGEALVRKLRFSMTDDEKEGIYADEAEKLWGKFRNDFENI |
| | ADHMNGDVIKELGKADMDFDEKIIDSEKKNASDLLYFSKMIYMLTYFLDGKEINDLLTTLISKFDNIK |
| | EFLKIMKSSAVDVE |
| UXKW01.1 | VIFMAKKNKMKPRELREAQKKARQLKAAEINNNAAPAIAAMPAAQVIAPAAEKKKSSVKAAGMKS |
| SEQ ID NO: | ILVSENKMYITSFGKGNSAVLEYEVDKVDNNNYNKTQLSSKDNSNIELGDVNEVNITFSSKHGFESG |
| 4885 | VEINTSNPTHRSGESSPVRWDMLGLKSELEKRFFGKTFDDNIHIQLIYNILDIEKILAVYVTNIVYALN |
| | NMLGIKGSESYDDFMGYLSARNTYEVFTHPDKSNLSDKVKGNIKKSFSTFNDLLKTKRLGYFGLEEP |
| | KTKDTRVSEAYKKRVYHMLAIVGQIRQCVFHDLSEHSEYDLYSFIDNSKKVYRECRETLNYLVDERF |
| | DSINKGFIQGNKVNISLLIDMMKDDYEADDIIHLYYDFIVLKSQKNLGFSIKKLREKMLDEYGFRFKD |
| | KQYDSVRSKMYKLMDFLLFCNYYRNDVVAGEALVRKLRFSMTDDEKEGIYADEAEKLWGKFRND |
| | FENIADHMNGDVIKELGKADMDFDEKILDSEKKNASDLLYFSKMIYMLTYFLDGKEINDLLTTLISKF |
| | DNIKEFLKIMKSSAVDVECELTAGYRLFND |
| UPFP01.1 | MAKKNKMKPRELREAQKKARQLKAAEINNNAVPAIAAMPAAEVIAPVAGKKKSSVKAAGMKSILV |
| SEQ ID NO: | SENKMYITSFGKGNSAVLEYEVDKVDDNDYNKTQLSSKGSSNIELHGVNEVNITFSSKHGFESGVEI |
| 4886 | NTSNPTHRSGESSPVRGDMLGLKSELEKRFFGKTFDDNIHIQLIYNILDIEKILAVYVTNIVYALNNML |
| | GIKDSESYDDFMGYLSAKNTYEVFTHPDKSNLSDKAKGNIKKSFSTFNDLLKTKRLGYFGLEEPKTK |
| | DTRVSQAYKKRVYHMLAIVGQIRQCVFHDLSEHSEYDLYSFIDNSKKVYRECRETLDYLVDERFDSI |
| | NKGFIQGNKVNISLLIDMMKGYEADDIIRLYYDFIVLKSQKNLGFSIKKLREKMLDEYGFRFKDKQY |
| | DSVRSKMYKLMDFLLFCNYYRNDVVAGEALVRKLRFSMTDDEKEGTYADEAEKLWGKFRNDFENI |
| | ADHMNGDAIKELGKADMDFDEKILDSEKKNASDLLYFSKMIYMLTYFLDGKEINDLLTTLISKFDNI |
| | KEFLKIMKSSAVDVECELTAGYKLFNDSQRITNELFIVKNIASMRKPAASAKLTMFRDALTILGIDDNI |
| | TDDRISEILKLKEKGKGIHGLRNFITNNVIESSRFVYLIKYANAQKIREVAKNEKVVMFVLGGIPDTQI |
| | ERYYKSCVEFPDMNSSLEAKRSELARMIKNISFDDFKNVKQQAKGRENVAKERAKAVIGLYLTVMY |
| | LLVKNLVNVNARYVIAIHCLERDFGLYKEIIPELASKNLKNDYRILSQTLCELCDKSPNLFLKKNERL |
| | RKCSLRAIFFSISIMQTAA |
| UPQO01.1 | MAKKNKMKPRELREAQKKARQLKAAEINNNAVPAIAAMPAAEVIAPVAGKKKSSVKAAGMKSILV |
| SEQ ID NO: | SENKMYITSFGKGNSAVLEYEVDKVDDNDYNKTQLSSKGSSNIELHGVNEVNITFSSKHGFESGVEI |
| 4887 | NTSNPTHRSGESSPVRGDMLGLKSELEKRFFGKTFDDNIHIQLIYNILDIEKILAVYVTNIVYALNNML |
| | GIKDSESYDDFMGYLSAKNTYEVFTHPDKSNLSDKAKGNIKKSFSTFNDLLKTKRLGYFGLEEPKTK |
| | DTRVSQAYKKRVYHMLAIVGQIRQCVFHDLSEHSEYDLYSFIDNSKKVYRECRETLDYLVDERFDSI |
| | NKGFIQGNKVNISLLIDMMKGYEADDIIRLYYDFIVLKSQKNLGFSIKKLREKMLDEYGFRFKDKQY |
| | DSVRSKMYKLMDFLLFCNYYRNDVVAGEALVRKLRFSMTDDEKEGIYADEAAKLWGKFRNDFENI |
| | ADHMNGDVTKELGKADMDFDEKILDSEKKNASDLLYFSKMIYMLTYFLDGKEINDLLTTLISKFDNI |
| | KEFLKIMKSSAVDVECELTAGYKLFNDSQRITNELFIVKNIASMRKPAASARLTMFRDALTI |
| OLNO01.1 | MAKKNKMKPRELREAQKKARQLKAAEINNNAAPAIAAMPAAEVIAPAAEKKKSSVKAAGMKSILV |
| SEQ ID NO: | SENKMYITSFGKGNSAVLEYEVDNNDYNKTQLSSKDNSNIELGDVDEVNITFSSKHGFGSGVEINTS |
| 4888 | NPTHRSGESSPVRWDMLGLKSELEKRFFGKTFDDNIHIQFIYNILDIEKILAVYVTNIVYALNNMLGIK |
| | DSESYDDFIGYLSARNTYKVFTHPDKSNLSDKAKGNIKKSFSTFNDLLKTKRLGYFGLEEPKTKDTR |
| | VLEAYKKRVYYMLAIVGQIRQCVFHDLSEHSEYDLYSFIDNSKKVYRECRETLDYLVDERFDSINKG |
| | FIQGNKVNISLLIDMMKGYEADDIIRLYYDFIVLKSQKNLGFSIKKLREKMLDEYGFRFKDKQYDSVR |
| | SKMYKLMDFLLFCNYYRNDVVAGEALVRKLRFSMTDDEKEGIYADEAAKLWVKF |
| OODI01.1 | MAKKNKMKPRELREAQKKARQLKAAEINNNAAPAIAAMPAAEVIAPAAEKKKSSVKAAGMKSILV |
| SEQ ID NO: | SENKMYITSFGKGNSAVLEYEVDNNDYNKTQLSSKDNSNIELGDVDEVNITFSSKHGFGSGVEINTS |
| 4889 | NPTHRSGESSPVRWDMLGLKSELEKRFFGKTFDDNIHIQFIYNILDIEKILAVYVTNIVYALNNMLGIK |
| | DSESYDDFIGYLSARNTYKVFTHPDKSNLSDKAKGNIKKSFSTFNDLLKTKRLGYFGLEEPKTKDTR |
| | VLEAYKKRVYYMLAIVGQIRQCVFHDLSEHSEYDLYSFIDNSKKVYRECRETLDYLVDERFDSINKG |
| | FIQGNKVNISLLIDMMKGYEADDIIRLYYDFIVLKSQKNLGFSIKKLREKMLDEYGFRFKDKQYDSVR |
| | SKMYKLMDFLLFCNYYRNDVVAGEALVRKLRFSMTDDEKEGIYADEAAKLWVKFRNDFENIADH |
| | MNGDVTKELGKADMDFDEKILDSEKKNASDLLYFSKMIYMLTYFLDGKEINDLLTTLISKFDNIKEFL |
| | KIMKSSAVDVECELTAGYKLFNDSQRITNELFIVKNIASMRKPAASAKLTMFRDALTILGIDDKITDD |
| | RISEILKLKEKGKGIHGLRNFITNNVTESSRFVYLIKYANAQKIREVAKNEKVVMFVLGGIPDTQIERY |
| | YKSCVEFPDMNSSLEVKRSELARMIKNISFDDFKNVKQQAKGRENVAKERAKAVIGLYLTVMYLLV |
| | KNLVNVNARYVIAIHCL |
| UZOD01.1 | VIFMAKKNKMKPRELREAQKKARQLKAAEINNNAVPAIAAMPAAEVIAPAAEKKKSSVKAAGMKS |
| SEQ ID NO: | ILVSENKMYITSFGKGNSAVLEYEVDNNDYNKTQLSSKDNSNIELGNVNEVNITFSSRRGFESGVEIN |
| 4890 | TSNPTHRSGESSSVRGDMLGLKSELEKRFFGKTFDDNIHIQLIYNILDIEKILAVYVTNIVYALNNMLG |
| | VKGSESYDDFMGYLSAQNTYYIFTHPDKSNLSDKVKGNIKKSLSKFNDLLKTKRLGYFGLEEPKTKD |
| | KRVSEAYKKRVYHMLAIVGQIRQCVFHDLSEHSEYDLYSFIDNSKKVYRECRETLDYLVDERFDSIN |
| | KGFIQGNKVNISLLIDMMKDYEADDIIRLYYDFIVLKSQKNLGFSIKKLREKMLDEYGFRFKDKQYD |
| | SVRSKMYKLMDFLLFCNYYRNDVVAGEALVRKLRFSMTDDEKEGIYADEAAKLWGKFRNDFENIA |
| | DHMNGDVTKELGKADMDFDEKILDSEKKNASDLLYFSKMIYMLTYFLDGKEINDLLTTLISKFDNIK |
| | EFLKIMKSSAVNVECELTAGYKLFNDSQRITNELFIVKNIASMRKPAASAKLTMFRDALTILGIDDKIT |
| | DDRISEILKLKEKGKGIHGLRNFITNNVIESSRFVYLIKYANAQKIREVAKNEKWMFVLGGIPDTQIE |
| | RYYKSCVEFPDMNSSLEVKRSELARMIKNISFDDFKNVKQQAKGRENVAKERAKAVIGLYLTVMYL |
| | LVKNLVNVNARYVIAIHCLERDFGLYKEIIPELASKNLKNDYRILSQTLCELCDNRDESPNLFLKKNK |
| | RLRKCVEVDINNADSSMTRKYRNCIAHLTVVRELKEYIGDIRTVDSYFSI |
| ULUI01.1 | MAKKNKMKPRELREAQKKARQLKAAEINNNAVPAIAAMPAAEAAAPAAEKKKSSVKAAGMKSIL |
| SEQ ID NO: | VSENKMYITSFGKGNSAVLEYEVDNNDYNKTQLSSKDNSNIELCDVDEVNITFSSKHGFESGVKINTS |
| 4891 | NPTHRSGESSSVRGDMLGLKSELEKRFFGKTFDDNIHIQLIYNILDIEKILAVYVTNIVYALNNMLGLE |
| | NESNNDFMGYLSAKNTYDVFTDPDESDLSKNIKGNIKKSLSKFNDLLKTKRLGYFGLEEPKTKDKRV |
| | SEAYKKRVYHMLAIVGQIRQSVFHDKSNELDEYLYSFIDIIDSEYRETLDYLIDERFDSINKGFIQGNK |
| | VNISLLIDMMKGYEADDIIRLYYDFIVLKSQKNLGFSIKKLREKMLDEYGFRFKDKQYDSVRSKMYK |
| | LMDFLLFCNYYRNDVVAGEALVRKLRFSMTDDEKEGIYADEAEKLWGKFRNDFENIADHMNGDVI |
| | KELGKADMDFDEKILDSEKKNASDLLYFSKMIYMLTYFLDGKEINDLLTTLISKFDNIKEFLKIMKSS |
| | AVDVECELTAGYKLFNDSQRITNELFIVKNIASMRKPAASAKLTMFRDALTILGIDDKITDDRISEILK |
| | LKEKGKGIHGLRNFITNNVIESSRFVYLIKYANAQKIREVAKNEKWMFVLGGIPDTQIERYYKSCVE |
| | SPDMNSSLEAKRRENVAKERAKAVIGLYLTVTYLLVKNLVNVNARYVIAIHCLERDFGLYKEIIPEL |
| | ASKNLKNDYRILSQTLCELCDDRDESPNLFLKKNKRLRKCVEVDINNADSSMTRKYRNCIAHLTVVR |
| | ELKEYIGDIRTVDSYFSIYHYVMQRCITKREDDTKQEEKIKYEDDLLKNHGYTKDFVKALNSSFGYNI |
| | PRFKNLSIEQLFDRNEYLTEK |
| UXSK01.1 | MAKKNKMKPRELREAQKKARQLKAAEINNNAVPAIAAMPAAEAAAPAAEKKKSSVKAAGMKSIL |
| SEQ ID NO: | VSENKMYITSFGKGNSAVLEYEVDNNDYNKTQLSSKDNSNIELCDVDEVNITFSSKHGFESGVKINTS |
| 4892 | NPTHRSGESSSVRGDMLGLKSELEKRFFGKTFDDNIHIQLIYNILDIEKILAVYVTNIVYALNNMLGLE |
| | NESNNDFMGYLSAKNTYDVFTDPDESDLSKNIKGNIKKSLSKFNDLLKTKRLGYFGLEEPKTKDKRV |
| | SEAYKKRVYHMLAIVGQIRQSVFHDKSNELDEYLYSFIDIIDSEYRETLDYLIDERFDSINKGFIQGNK |
| | VNISLLIDMMKGYEADDIIRLYYDFIVLKSQKNLGFSIKKLREKMLDEYGFRFKDKQYDSVRSKMYK |
| | LMDFLLFCNYYRNDVVAGEALVRKLRFSMTDDEKEGIYADEAEKLWGKFRNDFENIADHMNGDVI |
| | KELGKADMDFDEKILDSEKKNASDLLYFSKMIYMLTYFLDGKEINDLLTTLISKFDNIKEFLKIMKSS |
| | AVDVECELTAGYKLFNDSQRITNELFIVKNIASMRKPAASAKLTMFRDALTILGIDDKITDDRISEILK |
| | LKEKGKGIHGLRNFITNNVIESSRFVYLIKYANAQKIREVAKNEKWMFVLGGIPDTQIERYYKSCVE |
| | SPDMNSSLEAKRRENVAKERAKAVIGLYLTVTYLLVKNLVNVNARYVIAIHCLERDFGLYKEIIPEL |
| | ASKNLKNDYRILSQTLCELCDDRDESPNLFLKKNKRLRKCVEVDINNADSSMTRKYRNCIAHLTVVR |
| | ELKEYIGDIRTVDSYFSIYHYVMQRCITKREDDTKQEEKIKYEDDLLKNHGYTKDFVKALNSSFGYNI |
| | PRFKNLSIEQLFDRNEYLTEK |
| OYDX01.1 | MAKKNKMKPRERREAQKKARQLKAAEINNNAVPAIAAMPAAEAAAPAAEKKKSSVKAAGMKSIL |
| SEQ ID NO: | VSENKMYITSFGKGNSAVLEYEVDNNDYNKTQLSSKDNSNIELCDVDEVNITFSSKHGFESGVKINTS |
| 4893 | NPTHRSGESSSVRGDMLGLKSELEKRFFGKTFDDNIHIQLIYNILDIEKILAVYVTNIVYALNNMLGLE |
| | NESNNDFMGYLSAKNTYDVFTDPDESDLSKNIKGNIKKSLSKFNDLLKTKRLGYFGLEEPKTKDKRV |
| | SEAYKKRVYHMLAIVGQIRQSVFHDKSNELDEYLYSFIDIIDSEYRETLDYLLSLIHISEP |
| ORUY01.1 | MAKKNKMKPRELREAQKKARQLKAAEIKNNAVPAIAAMPAAEAAAPAVEKKKSSVKAAGMKSIL |
| SEQ ID NO: | VSENKMYITSFGKGNSAVLEYEVDNNDYNKTQLSSEDNSNIELCDVDEVNITFSSKHGFESGVEINTS |
| 4894 | NPTHRSGESSPVRGDMLGLKSELEKRFFGKTFDDNIHIQLIYNILDIEKILAVYVTNIVYALNNMLGEG |
| | GDESHDDFMGYLSAKNTYDVFTDPDESDLSKNIKGNIKKSLSKFNDLLKTKRLGYFGLEEPKTKDKR |
| | ASEAYKKRVYHMLAIVGQIRQSVFHDKSNELDEYLYSFIDIIDSEYRDTLDYLVDERFDSINKGFIQG |
| | NKVNISLLIDMMKGYEADDIIRLYYDFIVLKSQKNLGFSIKKLREKMLDEYGFRFKDKQYDSVRSKM |
| | YKLMDFLLFCNYYRKDVGAGEALVRKLRFSMTDEEKEGIYAYEAAKLWGKFRNDFENIADHMNG |
| | DVIKELGKADMDFDEKILDSEKKNASDLLYFSKMIYMLTYFLDGKEINDFLTTLISKFDNIKEFLKIM |
| | KSSAVDVECKLTAGYKLFNDSQRITNELFIVKNIASMRKPAASAKLTMFRDALTILGIDDKITDDRISE |
| | ILKLKEKGKGIHGLRNFITNNVIESSRFVYLIKYANAQKIREVAENEKVVMFVLGGIPDTQIERYYKSC |
| | VEFPDMNSSLEAKRSELARMIKNISFDDFKNVKQQAKGRENVAKERAKAVIGLYLTVMYLLVKNLV |
| | NVNARYVIAIHCLERDFGLYKEIIPELASKNLKNDYRILSQTLCELCDDCDESPNLFLKKNKRLRKCV |
| | EVDINNADSSMTRKYRNCIAHLTVVRELKEYIGDIRTVDSYFSIYHY |
| OGWT01.1 | VIFMAKKNKMKPRELREAQKKARQLKAAEINNNAVPAIAAMPAAEAAAPAAEKKKSSVKAAGMK |
| SEQ ID NO: | SILVSENKMYITSFGKGNSAVLEYEVDNNDYNKTQLSSKDNSNIELCDVDEVNITFSSKHGFESGVKI |
| 4895 | NTSNPTHRSGESSSVRGDMLGLKSELEKRFFGKTFDDNIHIQLIYNILDIEKILAVYVTNIVYALNNML |
| | GLENESNNDFMGYLSAKNTYDVFTDPDESDLSKNIKGNIKKSLSKFNDLLKTKRLGYFGLEEPKTKD |
| | KRVSEAYKKRVYHMLAIVGQIRQSVFHDKSNELDEYLYSFIDIIDSEYRETLDYLIDERFDSINKGFIQ |
| | GNKVNISLLIDMMKDDYEADDIIRLYYDFIVLKSQKNLGFSIKKLREKMLDEYGFRFKDKQYDSVRS |
| | KMYKLMDFLLFCNYYRNDVIAGEALVRKLRFSMTDDEKEGIYADEAAKLWGKFRNDFENIADHMN |
| | GDVIKELGKADMDFDEKILDSEKKNASDLLYFSKMIYMLTYFLDGKEINDLLTTLISKFDNIKEFLKI |
| | MKSSAVNVECELTAGYKLFNDSQRITNELFIVKNIASMRKPAASAKLTMFRDALTILGIDDKITDDRI |
| | SEILKLKEKGKGIHGLRNFITNNVIESSRFVYLIKYANAQKIREVAENEKVVMFVLGGIPDTQIERYYK |
| | SCVEFPDMNSSLEVKRSELARMIKNIRFDDFKNVKQQAKGRENVAKERAKAVIGLYLTVMYLLVKN |
| | LVNVNARYVIAIHCLERDFGLYKEIIPELASKNLKNDYRILSQTLCELCDNGDESPNLFLKKNRRLRK |
| | CVEVDINNADSSMTRKYRNCIAHLTVVRELKEYIGDIRTVDSYFSIYHYVMQRCITKREDDTK |
| OODB01.1 | MAKKNKMKPRELREAQKKARQLKAAEINNNAVPAIAAMPAAEAAAPAAEKKKSSVKAAGMKSIL |
| SEQ ID NO: | VSENKMYITSFGKGNSAVLEYEVDNNDYNKTQLSSKDNSNIELCDVDEVNITFSSKHGFESGVKINTS |
| 4896 | NPTHRSGESSSVRGDMLGLKSELEKRFFGKTFDDNIHIQLIYNILDIEKILAVYVTNIVYALNNMLGLE |
| | NESNNDFMGYLSAKNTYDLSKNIKGNIKKSLSKFNDLLKTKRLGYFGLEEPKTKDKRVSEAYKKRV |
| | YHMLAIVGQIRQSVFHDKSNELDEYLYSFIDIIDSEYRETLDYLIDERFDSINKGFIQGNKVNISLLIDM |
| | MKDDYEADDIIRLYYDFIVLKSQKNLGFSIKKLREKMLDEYGFRFKDKQYDSVRSKMYKLMDFLLF |
| | CNYYRNDVIAGEALVRKLRFSMTDDEKEGIYADEAAKLWGKFRNDFENIADHMNGDVIKELGKAD |
| | MDFDEKILDSEKKNASDLLYFSKMIYMLTYFLDGKEINDLLTTLISKFDNIKEFLKIMKSSAVNVECE |
| | LTAGYKLFNDSQRITNELFIVKNIASMRKPAASAKLTMFRDALTILGIDDKITDDRISEILKLKEKGKGI |
| | HGLRNFITNNVTESSRFVYLIKYANAQKIREVAENEKVVMFVLGGIPDTQIERYYKSCVEFPDMNSSL |
| | EVKRSELARMIKNIRFDDFKNVKQQAKGRENVAKERAKAVIGLYLTVMYLLVKNLVNVNARYVIAI |
| | HCLERDFGLYKEIIPELASKNLKNDYRILSQTLCELCDNGDESPNLFLKKNRRLRKCVEVDINNADSS |
| | MTRKYRNCIAHLTVVRELKEYIGDIRTVDSYFSIYHYAALYHEKGR |
| UPPV01.1 | MAKKNKMKPRELREAQKKARQLKVAEINNNAVPAIAAMPAAQVIAPAAEKKKSSVKAAGMKSILV |
| SEQ ID NO: | SENKMYITSFGKGNSAVLEYEVDNNDYNKTQLSSEGNSNIELGDVNEVNITFSSKRGFESGVEINTSN |
| 4897 | PTHRSGESSPVRGDMLGLKSELEKRFFGKTFDDNIHIQLIYNILDIEKILAVYVTNIVYALNNMLGEGD |
| | DESHDDFMGYLSAKNTYDVFTDPDESDLSKNIKGNIKKSFSTFNDLLKTKRLGYFGLEEPKTKDTRV |
| | SQAYKKRVYHMLAIVGQIRQSVFHDKSSKLDEDLYSFIDIIDSEYRDTLDYLVDERFDSINKGFIQGN |
| | KVNISLLIDMMKDDYEADDIIRLYYDFIVLKSQKNLGFSIKKLREKMLEEYGFRFKDKQYDSVRSKM |
| | YKIMDFLLFCNYYRNDVVAGEALVRKLRFSMTDDEKEGIYADEAAKLWGKFRNDFENIADHMNGD |
| | VIKELGKADMDFDEKILDSEKKNASDLLYFSKMIYMLTYFLDGKEINDLLTTLISKFDNIKEFLKIMK |
| | SSAVDVECELTAGYKLFNDSQRITNELFIVKNIASMRKPAASAKLTMFRDALTILGIDDGR |
| UPFB01.1 | VIFMAKKNKMKPRELREAQKKARQLKVAEINNNAVPAIAAMPAAQVIAPAAEKKKSSVKAAGMKS |
| SEQ ID NO: | ILVSENKMYITSFGKGNSAVLEYEVDNNDYNKTQLSSEGNSNIELGDVNEVNITFSSKRGFESGVEIN |
| 4898 | TSNPTHRSGESSPVRGDMLGLKSELEKRFFGKTFDDNIHIQLIYNILDIEKILAVYVTNIVYALNNMLG |
| | EGDDESHDDFMGYLSAKNTYDVFTDPDESDLSKNIKGNIKKSFSTFNDLLKTKRLGYFGLEEPKTKD |
| | TRVSQAYKKRVYHMLAIVGQIRQSVFHDKSSKLDEDLYSFIDIIDSEYRDTLDYLVDERFDSINKGFIQ |
| | GNKVNISLLIDMMKDDYEADDIIRLYYDFIVLKSQKNLGFSIKKLREKMLEEYGFRFKDKQYDSVRS |
| | KMYKIMDFLLFCNYYRNDVVAGEALVRKLRFSMTDDEKEGIYADEAAKLWGKFRNDFENIADHM |
| | NGDVIKELGKADMDFDEKILDSEKKNASDLLYFSKMIYMLTYFLDGKEINDLLTTLISKFDNIKEFLK |
| | IMKSSAVDVECELTAGYKLFNDSQRITNELFIVKNIASMRKPAASAKLTMFRDALTILGIDDNITDDRI |
| | SEILKLKEKGKGIHGLRNFITNNVIESSRFVYLIKYANAQKIREVAKNEKVVMFVLGGIPDTQIERYYK |
| | SCVEVPDMNSSLEAKRSELARMIK |
| UMEK01.1 | VIFMAKKNKMKPRELREAQKKARQLKAAEINNNAAPAIAAMPAAEVIAPAAEKKKSSVKAAGMKS |
| SEQ ID NO: | ILVSKNKMYITSFGKGNSAVLEYEVDNNDYNQTQLSSKNSSNIELHGVNEVNITFSSKHGFESGVEIN |
| 4899 | TSNPTHRSGESSPVRGDMLGLKSELEKRFFGKTFDDNIHIQLIYNILDIEKILAVYVTNIVYALNNMLG |
| | EGDDESHDDFMGYLSAKNTYDVFTDPDESDLSKNIKGNIKKSLSKFNDLLKTKRLGYFGLEEPKTKD |
| | TNALEAYKKRVYHMLAIVGQIRQSVFHDKSSKLDEDLYSFIDIIDSEYRETLDYLVDERFDSINKGFIQ |
| | GNKVNISLLIDMMKGYEADDIIRLYYDFIVLKSQKNLGFSIKKLREKMLEEYGYRFKDKQYDSVRSK |
| | MYKLMDFLLFCNYYRNDVVAGEALVRKLRFSMTDDEKEGIYADEASKLWGKFRNDFENIADHMN |
| | GDVIKELGKADMDFDEKILDSEKKNASDLLYFSKMIYMLTYFLDGKEINDLLTTLISKFDNIKEFLKI |
| | MKSSAVNVECELTTGYKLFNDSQRITNELFIVKNIASMRKPAASAKLTMFRDALTILGIDDKITDDRIS |
| | EILKLKEKGKGIHGLRNFITNNVIESSRFVYLIKYANAQKIREVAENEKWMFVLGGIPDTQIERYYKS |
| | CVEFPDMNSSLKAKRSELARMIKNIRFDDFKNVKQQAKGRENVAKERAKAVIGLYLTVMYLLVK |
| OVZR01.1 | VIFMAKKNKMKPRELREAQKKARQLKAAEINNNAVPAIAAMPAAEVIAPAAEKKKSSVKAAGMKS |
| SEQ ID NO: | ILVSENKMYITSFGKGNSAVLEYEVDKVDNDDYNKTQLSSKGSSNIELHGVNEVNITFSSKHGFESG |
| 4900 | VEISTSNPTHRSGESSPVRWDMLGLKSELEKRFFCKTFDDNIHIQLIYNILDIEKILAVYVTNIVYALNN |
| | MLGVKGSESHDDFIGYLSTNNIYDVFIDPDNSSLSDDKKANVRKSLSKFNVLLKTKRLGYFGLEEPK |
| | TKDTRASEAYKKRVYHMLAIVGQIRQCVFHDKSGAKRFDLYSFINNIDSEYRETLDYLVDERFDSIN |
| | KGFIEGNKINISLLIDMMKGYEADDIIRLYYDFIVLKSQKNLGFSIKKLRE |
| IMG_ | MAKKNKMKPRELREAQKKARQLKVAEINNNAAPAIAAMPAVEVIAPAAEKKKSSVKAAGMKSILV |
| 3300014744 | SENKMYITSFGKGNSAVLEYEVDNNDYNKTQLSSKDNSNIELGNVNEVNITFSSRRGFESGVEINTSN |
| SEQ ID NO: | PTHRSGESSSVRGDMLGLKSELEKRFFGKTFDDNIHIQLIYNILDIEKILAVYVTNIVYALNNMLGIKG |
| 4901 | SESYDDFMGYLSARNTYEVFTNPNGSTLSDDKKENIRKSLSKFNALLKTKRLGYFGLEEPKTKDTRV |
| | SQAYKKRVYHMLAIVGQIRQSVFHDKSSKLDEDLYSFIDIIDSEYRETLDYLVDERFDSINKGFVQGN |
| | KVNISLLIDMMKDDYEADDIIRLYYDFXXXXHCA |
| GCA_ | MPAAEVIAPAAEKKKSSVKAAGMKSILVSENKMYITSFGKGNSAVLEYEVDNNDYNQTQLSSEDSS |
| 003461775.1_ | NIELCGVTKVNITFSSKHGLESGVEINTSNPTHRSGESSPVRWDMLGLKSELEKRFFGKTFDDNIHIQL |
| ASM346177v1_ | IYNILDIEKILAVYVTNIVYALNNMLGIKKSESYDDFMGYLSARNTYEVFTHPDKSNLSDKAKGNIKK |
| genomic | SFSTFNDLLKTKRLGYFGLEEPKTKDTRVSQAYKKRVYHMLAIVGQIRQCVFHDKSGAKKFDLYSFI |
| SEQ ID NO: | NNIDSEYRETLDYLVDERFDSINKGFIQGNKVNISLLIDMMKGYKADDIIRLYYDFIVLKSQKNLGFSI |
| 4902 | KKLREKMLDEYGFRFKDKQYDSVRSKMYKLMDFLLFCNYYRNDVIAGEDLVRKLRFSMTDDEKEG |
| | IYADEAEKLWGKFRNDFENIADHMNGDVIKELGQADMDFDEKILDSEKKNASDLLYFSKMIYMLTY |
| | FLDGKEINDLLTTLISKFDNIKEFLKIMKSSAVDVECELTAGYKLFNDSQRITNELFIVKNIASMRKPA |
| | ASAKLTMFRDALTILGIDDKITDDRISEI |
| UZHM01.1 | MAKKNKMKPRELREAQKKARQLKAAEINNNAAPAIAAMPAAEVIAPAAEKKKSSVKAAGMKSIFV |
| SEQ ID NO: | SENKMYITSFGKGNSAVLEYEVDNNDYNQTQLSSEDSSNIELCGVTKVNITFSSKHGLESGVEINTSN |
| 4903 | PTHRSGESSPVRWDMLGLKSELEKRFFGKTFDDNIHIQLIYNILDIEKMLAVYVTNIVYALNNMLGIK |
| | KSESYDDFMGYLSARNTYEVFTHPDKSNLSDKVKGNIKKSFSTFNDLLKTKRLGYFGLEEPKTKDTR |
| | VLEAYKKRVYHMLAIVGQIRQCVFHDKSGAKRFDLYSFINNIDPEYRETLDYLVDERFDSINKDFIEG |
| | NKVNISLLIDMMKGYEADDIIRLY |
| OHYP01.1 | MAKKNKMKPRELREAQKKARQLKAAEINNNAAPAIAAMPAAEVIAPAAEKKKSSVKAAGMKSILV |
| SEQ ID NO: | SENKMYITSFGKGNSAVLEYEVDNNDYNQTQLSSKDNSNIQLGGVNEVNITFSSKHGFESGVEINTS |
| 4904 | NPTHRSGESSPVRGDMLGLKSELEKRFFGKTFDDNIHIQLIYNILDIEKILAVYVTNIVYALNNMLGV |
| | KGSESHDDFIGYLSTNNIYDVFIDPDNSSLSDDKKANVRKSLSKFNALLKTKRLGYFGLEEPKTKDNR |
| | VSQAYKKRVYHMLAIVGQIRQCVFHDKSGAKRFDLYSFINNIDPEYRDTLDYLVEERLKSINKDFIED |
| | NKVNISLLIDMMKGYEADDIIRLYYDFIVLKSQKNLGFSIKKLREKMLDEYGFKRTAWVLANTLHEL |
| | KWDGRFSYANKHWAEKIYIPTDLNHNSDFVVRSHPAVLDGFVSFYRKAVQALNLFGAEHCVGDRA |
| | EQDFTGKVLVLSPDTLKESCWSQENQLWYAHDGFGCSPHAIGRSVRCTCLGDGEMTRWNRDEFIGV |
| | LDEQFLPEWAQEKLAELTAPRQEETTTGEMKLE |
| UZMO01.1_2 | MMKKEGIYADEAAKLWGKFRNDFENIADHMNGEAIKELGKADMDFDEKILDSEKKNASDLLYFSK |
| SEQ ID NO: | MIYMLTYFLDGKEINDLLTTLISKFDNIKEFLKIMKSSAVDVECELTAGYKLFNDSQRITNELFIVKNI |
| 4905 | ASMRKPAASAKLTMFRDALTILGIDDNITDDRISEILKLKEKGKGIHGLRNFITNNVIESSRFVYLIKYA |
| | NAQKIREVAENEKVVMFVLGGIPDTQIERYYKSCVEFPDMNSSLEAKRSELARMIKNISFDDFKNVK |
| | QQAKGRENVAKERAKAVIGLYLTVMYLLVKNLVNVNARYVIAIHCLERDFGLYKEIIPELASKNLK |
| | NDYRILSQTLCELCDDRDESPNLFLKKNKRLRKCVEVDINNADSSMTRKYRNCIAHLTVVRELKEYI |
| | GDIRTVDSYFSIYHYVMQRCITKREDDTKQEDKIKYEDDLLKNHGYTKDFVKALNSPFGYNIPRFKN |
| | LSIEQLFDRNEYLTEK |
| OGZW01.1 | MMKKEGIYADEAAKLWGKFRNDFENIADHMNGEAIKELGKADMDFDEKILDSEKKNASDLLYFSK |
| SEQ ID NO: | MIYMLTYFLDGKEINDLLTTLISKFDNIKEFLKIMKSSAVDVECELTAGYKLFNDSQRITNELFIVKNI |
| 4906 | ASMRKPAASAKLTMFRDALTILGIDDNITDDRISEILKLKEKGKGIHGLRNFITNNVIESSRFVYLIKYA |
| | NAQKIREVAKNEKVVMFVLGGIPDTQIERYYKSCVEFPDMNSSLEAKRSELARMIKNIGFDDFKNVK |
| | QQAKGRENVAKERAKAVIGLYLTVMYLLVKNLVNVNARYVIAIHCLERDFGLYKEIIPELASKNLK |
| | NDYRILSQTLCELCDDRDESPNLFLKKNKRLRKCVEVDINNADSSMTRKYRNCIAHLTVVRELKEYI |
| | GDIRTVDSYFSIYHYVMQRCITKREDDTKQEDKIKYEDDLLKNHGYTKDFVKALNSPFGYNIPRFKN |
| | LSIEQLFDRNEYLTEK |
| OGEZ01.1 | VEINTSNPTHRSGESSSVRGDMLGLKSELEKRFFGKTFDDNIHIQLIYNILDIEKILAVYVTNIVYALNN |
| SEQ ID NO: | MLGIKDSESYDDFIGYLSARNTYKVFTHPDKSNLSDKVKGNIKKSFSTFNDLLKTKRLGYFGLEEPKT |
| 4907 | KDTRVSQAYKKRVYHMLAIVGQIRQSVFHDKSSKLDEDLYSFIDIIDPEYRETLDYLVDERFDSINKG |
| | FIQGNKVNISLLIDMMKGYEADDIIRLYYDFIVLKSQKNLGFSIKKLREKMLDEYGFRFKDKQYDSVR |
| | SKMYKLMDFLLFCNYYRNDVVAGEALVRKLRFSMTDDEKEGIYADEAAKLWGKFRNDFENIADH |
| | MNGEAIKELGKADMDFDEKILDSEKKNASDLLYFSKMIYMLTYFLDGKEINDLLTTLISKFDNIKEFL |
| | KIMKSSAVDVECELTAGYKLFNDSQRITNELFIVKNIASMRKPAASAKLTMFRDALTILGIDDNITDD |
| | RISEILKLKEKGKGIHGLRNFITNNVIESSRFVYLIKYANAQKIREVAKNEKVVMFVLGGIPDTQIERY |
| | YKSCVEFPDMNSSLEAKRSELARMIKNIGFDDFKNVKQQAKGRENVAKERAKAVIGLYLTVMYLLV |
| | KNLVNVNARYVIAIHCLERDFGLYKEIIPELASKNLKNDYRILSQTLCELCDDRDESPNLFLKKNKRL |
| | RKCVEVDINNADSSMTRKYRNCIAHLTVVRELKEYIGDIRTVDSYFSIYHYVMQRCITKREDDTKQE |
| | DKIKYEDDLLKNHGYTKDFVKALNSPFGYNIPRFKNLSIEQLFDRNEYLTEK |
| UPEE01.1 | MNGDVTKELGKADMDFDEKILDSEKKNASDLLYFSKMIYMLTYFLDGKEINDLLTTLISKFDNIKEFL |
| SEQ ID NO: | KIMKSSAVDVECELTAGYKLFNDSQRITNELFIVKNIASMRKPAASAKLTMFRDALTILGIDDKITDD |
| 4908 | RISEILKLKEKGKGIHGLRNFITNNVTESSRFVYLIKYANAQKIREVAENEKVVMFVLGGIPDTQIERY |
| | YKSCVEVPDMNSSLEAKRSELARMIKNISFDDFKNVKQQAKGRENVAKERAKAVIGLYLTVMYLLV |
| | KNLVNVNARYVIAIHCLERDFGLYKEIIPELASKNLKNDYRILSQTLCELCDDRDKSPNLFLKKNKRL |
| | RKCVEVDINNADSIMTRKYRNCIAHLTVVRELKEYIGDIRTVDSYFSIYHYVMQRCITKREDDTKQEE |
| | KIKYEDDLLKNHGYTKDFVKALNSPFGYNIPRFKNLSIEQLFDRNEYLTEK |
| OVTY01.1 | MNGDVTKELGKADMDFDEKILDSEKKNASDLLYFSKMIYMLTYFLDGKEINDLLTTLISKFDNIKEFL |
| SEQ ID NO: | KIMKSSAVDVECELTAGYKLFNDSQRITNELFIVKNIASMRKPAASAKLTMFRDALTILGIDDKITDD |
| 4909 | RISEILKLKEKGKGIHGLRNFITNNVTESSRFVYLIKYANAQKIREVAENEKVVMFVLGGIPDTQIERY |
| | YKSCVEVPDMNSSLEAKRSELARMIKNISFDDFKNVKQQAKGRENVAKERAKAVIGLYLTVMYLLV |
| | KNLVNVNARYVIAIHCLERDFGLYKEIIPELASKNLKNDYRILSQTLCELCDDRDKSPNLFLKKNKRL |
| | RKCVEVDINNADSIMTRKYRNCIAHLTVVRELKEYIGDIRTVDSYFSIYHYVMQRCITKREDDTKQEE |
| | KIKYEDDLLKNHGYTKDFVKALNSPFGYNIPRFKNLSIEQLFDRNEYLTEK |
| OOCM01.1 | MNGDVTKELGKADMDFDEKILDSEKKNASDLLYFSKMIYMLTYFLDGKEINDLLTTLISKFDNIKEFL |
| SEQ ID NO: | KIMKSSAVDVECELTAGYKLFNDSQRITNELFIVKNIASMRKPAASAKLTMFRDALTILGIDDKITDD |
| 4910 | RISEILKLKEKGKGIHGLRNFITNNVTESSRFVYLIKYANAQKIREVAENEKVVMFVLGGIPDTQIERY |
| | YKSCVEVPDMNSSLEAKRSELARMIKNISFDDFKNVKQQAKGRENVAKERAKAVIGLYLTVMYLLV |
| | KNLVNVNARYVIAIHCLERDFGLYKEIIPELASKNLKNDYRILSQTLCELCDDRDKSPNLFLKKNKRL |
| | RKCVEVDINNADSIMTRKYRNCIAHLTVVRELKEYIGDIRTVDSYFSIYHYVMQRCITKREDDTKQEE |
| | KIKYEDDLLKNHGYTKDFVKALNSPFGYNIPRFKNLSIEQLFDRNEYLTEK |
| OHAE01.1_2 | MNGDVTKELGKADMDFDEKILDSEKKNASDLLYFSKMIYMLTYFLDGKEINDLLTTLISKFDNIKEFL |
| SEQ ID NO: | KIMKSSAVDVECELTAGYKLFNDSQRITNELFIVKNIASMRKPAASAKLTMFRDALTILGIDDKITDD |
| 4911 | RISEILKLKEKGKGIHGLRNFITNNVTESSRFVYLIKYANAQKIREVAENEKVVMFVLGGIPDTQIERY |
| | YKSCVEVPDMNSSLEAKRSELARMIKNISFDDFKNVKQQAKGRENVAKERAKAVIGLYLTVMYLLV |
| | KNLVNVNARYVIAIHCLERDFGLYKEIIPELASKNLKNDYRILSQTLCELCDDRDKSPNLFLKKNKRL |
| | RKCVEVDINNADSIMTRKYRNCIAHLTVVRELKEYIGDIRTVDSYFSIYHYVMQRCITKREDDTKQEE |
| | KIKYEDDLLKNHGYTKDFVKALNSPFGYNIPRFKNLSIEQLFDRNEYLTEK |
| OWRW01.1 | MNGDVTKELGKADMDFDEKILDSEKKNASDLLYFSKMIYMLTYFLDGKEINDLLTTLISKFDNIKEFL |
| SEQ ID NO: | KIMKSSAVDVECELTAGYKLFNDSQRITNELFIVKNIASMRKPAASAKLTMFRDALTILGIDDKITDD |
| 4912 | RISEILKLKEKGKGIHGLRNFITNNVTESSRFVYLIKYANAQKIREVAENEKVVMFVLGGIPDTQIERY |
| | YKSCVEVPDMNSSLEAKRSELARMIKNISFDDFKNVKQQAKGRENVAKERAKAVIGLYLTVMYLLV |
| | KNLVNVNARYVIAIHCLERDFGLYKEIIPELASKNLKNDYRILSQTLCELCDDRDKSPNLFLKKNKRL |
| | RKCVEVDINNADSIMTRKYRNCIAHLTVVRELKEYIGDIRTVDSYFSIYHYVMQRCITKREDDTKQEE |
| | KIKYEDDLLKNHGYTKDFVKALNSPFGYNIPRFKNLSIEQLFDRNEYLTEK |
| OGHH01.1 | MNGDVTKELGKADMDFDEKILDSEKKNASDLLYFSKMIYMLTYFLDGKEINDLLTTLISKFDNIKEFL |
| SEQ ID NO: | KIMKSSAVDVECELTAGYKLFNDSQRITNELFIVKNIASMRKPAASAKLTMFRDALTILGIDDKITDD |
| 4913 | RISEILKLKEKGKGIHGLRNFITNNVTESSRFVYLIKYANAQKIREVAENEKVVMFVLGGIPDTQIERY |
| | YKSCVEVPDMNSSLEAKRSELARMIKNISFDDFKNVKQQAKGRENVAKERAKAVIGLYLTVMYLLV |
| | KNLVNVNARYVIAIHCLERDFGLYKEIIPELASKNLKNDYRILSQTLCELCDDRDKSPNLFLKKNKRL |
| | RKCVEVDINNADSIMTRKYRNCIAHLTVVRELKEYIGDIRTVDSYFSIYHYVMQRCITKREDDTKQEE |
| | KIKYEDDLLKNHGYTKDFVKALNSPFGYNIPRFKNLSIEQLFDRNEYLTEK |
| OGOM01.1_2 | MRLMISYAFIMISLCLNLRKISVFSIKKLREKMLDEYGFRFKDKQYDSVRSKMYKLMDFLLFCNYYR |
| SEQ ID NO: | NDVAAGEALVRKLRFSMTDDEKEGIYADEAAKLWGKFRNDFENIADHMNGDVIKELGKADMDFD |
| 4914 | EKILDSEKKNASDLLYFSKMIYMLTYFLDGKEINDLLTTLISKFDNIKEFLKIMKSSAVDVECELTAGY |
| | KLFNDSQRITNELFIVKNIASMRKPAASAKLTMFRDALTILGIDDNITDDRISEILKLKEKGKGIHGLR |
| | NFITNNVIESSRFVYLIKYANAQKIREVAKNEKVVMFVLGGIPDTQIERYYKSCVEFPDMNSSLEAKR |
| | SELARMIKNISFDDFKNVKQQAKGRENVAKERAKAVIGLYLTVMYLLVKNLVNVNARYVIAIHCLE |
| | RDFGLYKEIIPELASKNLKNDYRILSQTLCELCDERDKSPNLFLKKNERLRKCVEVDINNADSSMTRK |
| | YRNCIAHLTVVRELKEYIGDIRTVDSYFSIYHYVMQRCITKREDDKKQEEKIKYEDDLLKNHGYTKD |
| | FVKALNSPFGYNIPRFKNLSIEQLFDRNEYLTEK |
| UPFO01.1_2 | MRLMISYAFIMISLCLNLRKISVFSIKKLREKMLDEYGFRFKDKQYDSVRSKMYKLMDFLLFCNYYR |
| SEQ ID NO: | NDVAAGEALVRKLRFSMTDDEKEGIYADEAAKLWGKFRNDFENIADHMNGDVIKELGKADMDFD |
| 4915 | EKILDSEKKNASDLLYFSKMIYMLTYFLDGKEINDLLTTLISKFDNIKEFLKIMKSSAVDVECELTAGY |
| | KLFNDSQRITNELFIVKNIASMRKPAASAKLTMFRDALTILGIDDNITDDRISEILKLKEKGKGIHGLR |
| | NFITNNVIESSRFVYLIKYANAQKIREVAKNEKVVMFVLGGIPDTQIERYYKSCVEFPDMNSSLEAKR |
| | SELARMIKNISFDDFKNVKQQAKGRENVAKERAKAVIGLYLTVMYLLVKNLVNVNARYVIAIHCLE |
| | RDFGLYKEIIPELASKNLKNDYRILSQTLCELCDERDKSPNLFLKKNERLRKCVEVDINNADSSMTRK |
| | YRNCIAHLTVVRELKEYIGDIRTVDSYFSIYHYVMQRCITKREDDKKQEEKIKYEDDLLKNHGYTKD |
| | FVKALNSPFGYNIPRFKNLSIEQLFDRNEYLTEK |
| OVZV01.1_2 | MRLMISYAFIMISLCLNLRKISVFSIKKLREKMLDEYGFRFKDKQYDSVRSKMYKLMDFLLFCNYYR |
| SEQ ID NO: | NDVAAGEALVRKLRFSMTDDEKEGIYADEAAKLWGKFRNDFENIADHMNGDVIKELGKADMDFD |
| 4916 | EKILDSEKKNASDLLYFSKMIYMLTYFLDGKEINDLLTTLISKFDNIKEFLKIMKSSAVDVECELTAGY |
| | KLFNDSQRITNELFIVKNIASMRKPAASAKLTMFRDALTILGIDDNITDDRISEILKLKEKGKGIHGLR |
| | NFITNNVIESSRFVYLIKYANAQKIREVAKNEKWMFVLGGIPDTQIERYYKSCVEFPDMNSSLEAKR |
| | SELARMIKNISFDDFKNVKQQAKGRENVAKERAKAVIGLYLTVMYLLVKNLVNVNARYVIAIHCLE |
| | RDFGLYKEIIPELASKNLKNDYRILSQTLCELCDERDKSPNLFLKKNERLRKCVEVDINNADSIMTRK |
| | YRNCIAHLTVVRELKEYIGDIRTVDSYFSIYHYVMQRCITKREDDTKQEEKIKYEDDLLKNHGYTKD |
| | FVKALNSPFGYNIPRFKNLSIEQLFDRNEYLTEK |
| OGGK01.1_2 | MRLMISYAFIMISLCLNLRKISVFSIKKLREKMLDEYGFRFKDKQYDSVRSKMYKLMDFLLFCNYYR |
| SEQ ID NO: | NDVAAGEALVRKLRFSMTDDEKEGIYADEAAKLWGKFRNDFENIADHMNGDVIKELGKADMDFD |
| 4917 | EKILDSEKKNASDLLYFSKMIYMLTYFLDGKEINDLLTTLISKFDNIKEFLKIMKSSAVDVECELTAGY |
| | KLFNDSQRITNELFIVKNIASMRKPAASAKLTMFRDALTILGIDDNITDDRISEILKLKEKGKGIHGLR |
| | NFITNNVIESSRFVYLIKYANAQKIREVAKNEKWMFVLGGIPDTQIERYYKSCVEFPDMNSSLEAKR |
| | SELARMIKNISFDDFKNVKQQAKGRENVAKERAKAVIGLYLTVMYLLVKNLVNVNARYVIAIHCLE |
| | RDFGLYKEIIPELASKNLKNDYRILSQTLCELCDERDKSPNLFLKKNERLRKCVEVDINNADSIMTRK |
| | YRNCIAHLTVVRELKEYIGDIRTVDSYFSIYHYVMQRCITKREDDTKQEEKIKYEDDLLKNHGYTKD |
| | FVKALNSPFGYNIPRFKNLSIEQLFDRNEYLTEK |
| OZEI01.12 | MFRDALTILGIDDKITDDRISEILKLKEKGKGIHGLRNFITNNVIESSRFVYLIKYANAQKIREVAENEK |
| SEQ ID NO: | VVMFVLGGIPDTQIERYYKSCVEFPDMNSSLEAKRSELARMIKNISFDDFKNVKQQAKGRENVAKER |
| 4918 | AKAVIGLYLTVMYLLVKNLVNVNARYVIAIHCLERDFGLYKEIIPELASKNLKNDYRILSQTLCELCD |
| | DRDESPNLFLKKNKRLRKCVEVDINNADSSMTRKYRNCIAHLTVVRELKEYIGDIRTVDSYFSIYHY |
| | VMQRCITKREDDTKQEEKTKYEDDLLKNHGYTKDFVKALNSPFGYNIPRFKNLSIEQLFDRNEYLTE |
| | K |
| UAJR01.1 | MFRDALTILGIDDKITDDRISEILKLKEKGKGIHGLRNFITNNVIESSRFVYLIKYANAQKIREVAENEK |
| SEQ ID NO: | VVMFVLGGIPDTQIERYYKSCVEFPDMNSSLEAKRSELARMIKNISFDDFKNVKQQAKGRENVAKER |
| 4919 | AKAVIGLYLTVMYLLVKNLVNVNARYVIAIHCLERDFGLYKEIIPELASKNLKNDYRILSQTLCELCD |
| | DRDESPNLFLKKNKRLRKCVEVDINNADSSMTRKYRNCIAHLTVVRELKEYIGDIRTVDSYFSIYHY |
| | VMQRCITKREDDTKQEEKTKYEDDLLKNHGYTKDFVKALNSPFGYNIPRFKNLSIEQLFDRNEYLTE |
| | K |
| OYBT01.1 | VEINTSNPTHRSGESSPVRGDMLGLKSELEKRFFGKNFDDNIHIQLIYNILDIEKILAVYVTNIVYALN |
| SEQ ID NO: | NMLGEGDESNYDFMGYLSTFNTYKVFTNPNGSTLSDDKKENIRKSLSKFNALLKTKRLGYFGLEEPK |
| 4920 | TKDKRVSEAYKKRVYHMLAIVGQIRQSVFHDKSNELDEYLYSFIDIIDSEYRDTLDYLVDERFDSINK |
| | GFVQGNKVNISLLIDMMKGYEADDIIRLYYDFIVLKSQKNLGFSIKKLREKMLDEYGFRFKDKQYDS |
| | VRSKMYKLMDFLLFCNYYRNDIAAGEALVRKLRFSMTDDEKEGIYADEAEKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVDVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKWMFVLGGIPDTQIER |
| | YYKSCVEFPDMNSSLEAKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNRR |
| | LRKCVEVDINNADSSMTRKYRNCIAHLTVVRELKEYIGDIRTVDSYFSIYHYVMQRCITKREDDTKQ |
| | EDKIKYEDDLLKNHGYTKDFVKALNSPFGYNIPRFKNLSIEQLFDRNEYLTEK |
| OGTB01.12 | MFRDALTILGIDDKITDDRISEILKLKEKGKGIHGLRNFITNNVIESSRFVYLIKYANAQKIREVAKNEK |
| SEQ ID NO: | VVMFVLGGIPDTQIERYYKSCVEFPDMNSSLEVKRSELARMIKNIRFDDFKNVKQQAKGRENVAKE |
| 4921 | RAKAVIGLYLTVMYLLVKNLVNVNARYVIAIHCLERDFGLYKEIIPELASKNLKNDYRILSQTLCELC |
| | DDRDESPNLFLKKNKRLRKCVEVDINNADSSMTRKYRNCIAHLTVVRELKKYIGDIRTVDSYFSIYH |
| | YVMQRCITKREDDTKQEEKIKYEDDLLKNHGYTKDFVKALNSPFGYNIPRFKNLSIEQLFDRNEYLT |
| | EK |
| OOSV01.1 | MKSILVSENKMYITSFGKGNSAVLEYEVDKVDNNNYNKTQLSSKDNSNIELGDVNEVNITFSSKRGN |
| SEQ ID NO: | ESGVEINTSNPTHRSGESSPVRWDMLGLKSELEKRFFGKTFDDNIHIQLIYNILDIEKILAVYVTNIVYA |
| 4922 | LNNMLGIKGSESYDDFMGYLSARNTYEVFTHPDKSNLSDKVKGNIKKSLSKFNDLLKTKRLGYFGL |
| | EEPKTKDTRVSQAYKKRVYHMLAIVGQIRQCVFHDKSGAKRFDLYSFINNIDPEYRDTLDYLVEERL |
| | KSINKDFIEGNKVNISLLIDMMKGYEADDIIRLYYDFIVLKSQKNLGFSIKKLREKMLEEYGYRFKDK |
| | QYDSVRSKMYKLMDFLLFCNYYRNDVVAGEALVRKLRFSMTDDEKEGIYADEASKLWGKFRNDF |
| | ENIADHMNGDVIKELGKADMDFDEKILDSEKKNASDLLYFSKMIYMLTYFLDGKEINDLLTTLISKF |
| | DNIKEFLKIMKSSAVDVECELTAGYKLFNDSQRITNELFIVKNIASMRKPAASAKLTMFRDALTILGID |
| | DKITDDRISEILKLKEKGKGIHGLRNFITNNVTESSRFVYLIKYANAQKIREVAKNEKWMFVLGGIPD |
| | TQIERYYKSCVEFPDMNSSLEAKRSELARMIKNIRFDDFKNVKQQAKGRENVAKERAKAVIGLYLT |
| | VMYLLVKNLVNVNARYVIAIHCLERDFGLYKEIIPELASKNLKNDYRILSQTLCELCDDRDESPNLFL |
| | KKNKRLRKCVEVDINNADSSMTRKYRNCIAHLTVVRELKEYIGDIRTVDSYFSIYHYVMQRCITKRE |
| | DDTKQEEKIKYEDDLLKNHGYTKDFVKALNSPFGYNIPRFKNLSIEQLFDRNEYLTEK |
| UBVQ01.1 | VEINTSNPTHRSGESSSVRGDMLGLKSELEKRFFGKTFDDNIHIQLIYNILDIEKILAVYVTNIVYALNN |
| SEQ ID NO: | MLGVKGSESYDDFMGYLSAQNTYYIFTHPDKSNLSDKVKGNIKKSLSKFNDLLKTKRLGYFGLEEP |
| 4923 | KTKDTRVSQAYKKRVYHMLAIVGQIRQCVFHDKSGAKRFDIYSFINNIDPEYRETLDYLVDERFDSIN |
| | KGFIQGNKVNISLLIDMMKDYEADDIIRLYYDFIVLKSQKNLGFSIKKLREKMLDEYGFRFKDKQYD |
| | SVRSKMYKLMDFLLFCNYYRNDVVAGETLVRKLRFSMTDDEKEGIYADEAAKLWGKFRNDFENIA |
| | DHMNGDVTKELGKADMDFDEKILDSEKKNASDLLYFSKMIYMLTYFLDGKEINDLLTTLISKFDNIK |
| | EFLKIMKSSAVDVECELTEGYKLFNDSQRITNELFIVKNIASMRKPAASAKLTMFRDALTILGIDDKIT |
| | DDRISEILKLKEKGKGIHGLRNFITNNVIESSRFVYLIKYANAQKIREVAENEKVVMFVLGGIPDTQIE |
| | RYYKSCVEFPDMNSSLEAKRSELARMIKNIRFDDFKNVKQQAKGRENVAKERAKAVIGLYLTVMYL |
| | LVKNLVNVNARYVIAIHCLERDFGLYKEIIPELASKNLKNDYRILSQTLCELCDDRDESPNLFLKKNK |
| | RLRKCVEVDINNADSNMTRKYRNCIAHLTVVRELKEYIGDIRTVDSYFSIYHYVMQRCITKREDDTK |
| | QEEKIKYEDDLLKNHGYTKDFVKALNSPFGYNIPRFKNLSIEQLFDRNEYLTEK |
| UZPY01.1 | MMKDYEADDIIRLYYDFIVLKSQKNLGFSIKKLREKMLDEYGFRFKDKQYDSVRSKMYKLMDFLLF |
| SEQ ID NO: | CNYYRNDVVAGETLVRKLRFSMTDDEKEGIYADEAAKLWGKFRNDFENIADHMNGDVIKELGKAD |
| 4924 | MDFDEKILDSEKKNASDLLYFSKMIYMLTYFLDGKEINDLLTTLISKFDNIKEFLKIMKSSAVDVECE |
| | LTEGYKLFNDSQRITNELFIVKNIASMRKPAASAKLTMFRDALTILGIDDKITDDRISEILKLKEKGKGI |
| | HGLRNFITNNVTESSRFVYLIKYANAQKIREVAKNEKVVMFVLGGIPDTQIERYYKSCVEFPDMNSSL |
| | EAKRSELARMIKNIRFDDFKNVKQQAKGRENVAKERAKAVIGLYLTVMYLLVKNLVNVNARYVIAI |
| | HCLERDFGLYKEIIPELASKNLKNDYRILSQTLCELCDDRDESPNLFLKKNKRLRKCVEVDINNADSS |
| | MTRKYRNCIAHLTVVRELKEYIGDIRTVDSYFSIYHYVMQRCITKREDDTKQEEKIKYEDVLLKNHG |
| | YTKDFVKALNSPFGYNIPRFKNLSIEQLFDRNEYLTEK |
| ULVV01.1 | VEINTSNPTHRSGESSPVRGDMLGLKSELEKRFFGKTFDDNIHIQLIYNILDIEKILAVYVTNIVYALNN |
| SEQ ID NO: | MLGEGDESNYDFMGYLSTFNTYKVFTNPNGSTLSDDKKENIRKSLSKFNALLKTKRLGYFGLEEPKT |
| 4925 | KDNRVSEAYKKRVYHMLAIVGQIRQCVFHDKSGAKRFDLYSFINNIDPEYRETLDYLVDERFDSINK |
| | GFIQGNKVNISLLIDMMKDDYEADDIIRLYYDFIVLKSQKNLGFSIKKLREKMLEEYGFRFKDKQYDS |
| | VRSKMYKLMDFLLFCNYYRNDVVAGEALVRKLRFSMTDDEKEGIYADEAAKLWGKFRNDFENIAD |
| | HMNGDVTKELGQADMDFDEKILDSEKKNASDLLYFSKMIYMLTYFLDDKEINDLLTTLISKFDNIKEF |
| | LKIMKSSAVDVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKWMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKRENDTKQ |
| | EEKIKYEDDLLKNHGYTKDFVKALNSPFGYNIPRFKNLSIEQLFDRNEYLTEK |
| UXRQ01.1 | VEINTSNPTHRSGESSPVRGDMLGLKSELEKRFFGKTFDDNIHIQLIYNILDIEKILAVYVTNIVYALNN |
| SEQ ID NO: | MLGEGDESNYDFMGYLSTFNTYKVFTNPNGSTLSDDKKENIRKSLSKFNALLKTKRLGYFGLEEPKT |
| 4926 | KDNRVSEAYKKRVYHMLAIVGQIRQCVFHDKSGAKRFDLYSFINNIDPEYRETLDYLVDERFDSINK |
| | GFIQGNKVNISLLIDMMKDDYEADDIIRLYYDFIVLKSQKNLGFSIKKLREKMLEEYGFRFKDKQYDS |
| | VRSKMYKLMDFLLFCNYYRNDVVAGEALVRKLRFSMTDDEKEGIYADEAAKLWGKFRNDFENIAD |
| | HMNGDVTKELGQADMDFDEKILDSEKKNASDLLYFSKMIYMLTYFLDDKEINDLLTTLISKFDNIKEF |
| | LKIMKSSAVDVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKRENDTKQ |
| | EEKIKYEDDLLKNHGYTKDFVKALNSPFGYNIPRFKNLSIEQLFDRNEYLTEK |
| UZLX01.1 | VEINTSNPTHRSGESSPVRGDMLGLKSELEKRFFGKTFDDNIHIQLIYNILDIEKILAVYVTNIVYALNN |
| SEQ ID NO: | MLGEGDESNYDFMGYLSTFNTYKVFTNPNGSTLSDDKKENIRKSLSKFNALLKTKRLGYFGLEEPKT |
| 4927 | KDNRVSEAYKKRVYHMLAIVGQIRQCVFHDKSGAKRFDLYSFINNIDPEYRETLDYLVDERFDSINK |
| | GFIQGNKVNISLLIDMMKDDYEADDIIRLYYDFIVLKSQKNLGFSIKKLREKMLEEYGFRFKDKQYDS |
| | VRSKMYKLMDFLLFCNYYRNDVVAGEALVRKLRFSMTDDEKEGIYADEAAKLWGKFRNDFENIAD |
| | HMNGDVTKELGQADMDFDEKILDSEKKNASDLLYFSKMIYMLTYFLDDKEINDLLTTLISKFDNIKEF |
| | LKIMKSSAVDVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKRENDTKQ |
| | EEKIKYEDDLLKNHGYTKDFVKALNSPFGYNIPRFKNLSIEQLFDRNEYLTEK |
| OWBH01.1 | VEINTSNPTHRSGESSPVRGDMLGLKSELEKRFFGKTFDDNIHIQLIYNILDIEKILAVYVTNIVYALNN |
| SEQ ID NO: | MLGEGDESNYDFMGYLSTFNTYKVFTNPNGSTLSDDKKENIRKSLSKFNALLKTKRLGYFGLEEPKT |
| 4928 | KDNRVSEAYKKRVYHMLAIVGQIRQCVFHDKSGAKRFDLYSFINNIDPEYRETLDYLVDERFDSINK |
| | GFIQGNKVNISLLIDMMKDDYEADDIIRLYYDFIVLKSQKNLGFSIKKLREKMLEEYGFRFKDKQYDS |
| | VRSKMYKLMDFLLFCNYYRNDVVAGEALVRKLRFSMTDDEKEGIYADEAAKLWGKFRNDFENIAD |
| | HMNGDVTKELGQADMDFDEKILDSEKKNASDLLYFSKMIYMLTYFLDDKEINDLLTTLISKFDNIKEF |
| | LKIMKSSAVDVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKRENDTKQ |
| | EEKIKYEDDLLKNHGYTKDFVKALNSPFGYNIPRFKNLSIEQLFDRNEYLTEK |
| UPPO01.1 | VEINTSNPTHRSGESSPVRGDMLGLKSELEKRFFGKTFDDNIHIQLIYNILDIEKILAVYVTNIVYALNN |
| SEQ ID NO: | MLGEGDESNYDFMGYLSTFNTYKVFTNPNGSTLSDDKKENIRKSLSKFNALLKTKRLGYFGLEEPKT |
| 4929 | KDNRVSEAYKKRVYHMLAIVGQIRQCVFHDKSGAKRFDLYSFINNIDPEYRETLDYLVDERFDSINK |
| | GFIQGNKVNISLLIDMMKDDYEADDIIRLYYDFIVLKSQKNLGFSIKKLREKMLEEYGFRFKDKQYDS |
| | VRSKMYKLMDFLLFCNYYRNDVVAGEALVRKLRFSMTDDEKEGIYADEAAKLWGKFRNDFENIAD |
| | HMNGDVTKELGQADMDFDEKILDSEKKNASDLLYFSKMIYMLTYFLDDKEINDLLTTLISKFDNIKEF |
| | LKIMKSSAVDVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKWMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSSMTRKYRNCIAHITVVRELKEYIGDIRTVDSYFSIYHYVMQRCITKREDDTKQE |
| | EKIKYEDDLLKNHGYTKDFVKALNSPFGYNIPRFKNLSIEQLFDRNEYLTEK |
| UPPX01.1 | MLAIVGQIRQCVFHDKSGAKRFDLYSFINNIDPEYRETLDYLVDERFDSINKGFIQGNKVNISLLIDM |
| SEQ ID NO: | MKDDYEADDIIRLYYDFIVLKSQKNLGFSIKKLREKMLEEYGFRFKDKQYDSVRSKMYKLMDFLLF |
| 4930 | CNYYRNDVVAGEALVRKLRFSMTDDEKEGIYADEAAKLWGKFRNDFENIADHMNGDVIKELGQA |
| | DMDFDEKILDSEKKNASDLLYFSKMIYMLTYFLDDKEINDLLTTLISKFDNIKEFLKIMKSSAVDVEC |
| | ELTAGYKLFNDSQRITNELFIVKNIASMRKPAASAKLTMFRDALTILGIDDKITDDRISEILKLKEKGK |
| | GIHGLRNFITNNVIESSRFVYLIKYANAQKIREVAENEKVVMFVLGGIPDTQIERYYKSCVEFPDMNS |
| | SLEAKRSELARMIKNIRFDDFKNVKQQAKGRENVAKERAKAVIGLYLTVMYLLVKNLVNVNARYV |
| | IAIHCLERDFGLYKEIIPELASKNLKNDYRILSQTLCELCDDRDESPNLFLKKNKRLRKCVEVDINNAD |
| | SNMTRKYRNCIAHLTVVRELKEYIGDIRTVDSYFSIYHYVMQRCITKRENDTKQEEKIKYEDDLLKN |
| | HGYTKDFVKALNSPFGYNIPRFKNLSIEQLFDRNEYLTEK |
| UPLT01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4931 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OLRI01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4932 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OOZT01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4933 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| UYFS01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4934 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OROU01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4935 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVTESSRFVYLIKYANAQKIREVAENEKWMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OWSK01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4936 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| GCA_ | VEINTSNPTHRSGESSPVRGDMLGLKSELEKRFFGKTFDDNIHIQLIYNILDIEKILAVYVTNIVYALNN |
| 003462325.1_ | MLGVKGSESHDDFIGYLSTNNTYDVFIDPDNSSLSDDKKANVRKSLSKFNVLLKTKRLGYFGLEEPK |
| ASM346232v1_ | TKDNRVSEAYKKRVYHMLAIVGQIRQCVFHDKSGAKRFDLYSFINNIDPEYRDTLDYLVEERLKSIN |
| genomic | KDFIQGNKVNISLLIDMMKGYEADDIIRLYYDFIVLKSQKNLGFSIKKLREKMLEEYGFRFKDKQYDS |
| SEQ ID NO: | VRSKMYKLMDFLLFCNYYRNDIAAGEALVRKLRFSMTDDEKEGLYADEAAKLWGKFRNDFENIAD |
| 4937 | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| UZTE01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4938 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OPQO01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4939 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| IMG_ | VEINTSNPTHRSGESSPVRGDMLGLKSELEKRFFGKTFDDNIHIQLIYNILDIEKILAVYVTNIVYALNN |
| 3300007801 | MLGVKGSESHDDFIGYLSTNNTYDVFIDPDNSSLSDDKKANVRKSLSKFNVLLKTKRLGYFGLEEPK |
| SEQ ID NO: | TKDNRVSEAYKKRVYHMLAIVGQIRQCVFHDKSGAKRFDLYSFINNIDPEYRDTLDYLVEERLKSIN |
| 4940 | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OIZQ01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4941 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKWMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| UMIT01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4942 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| UZTP01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4943 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OWDU01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4944 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OWTO01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4945 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| UXSM01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4946 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKWMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OFPS01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4947 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OWFE01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4948 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| ULVY01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4949 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| UZPU01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4950 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| UZUM01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4951 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OVTQ01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4952 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVTESSRFVYLIKYANAQKIREVAENEKWMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OWCH01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4953 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OOCX01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4954 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OGFJ01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4955 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| USWB01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4956 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| GCA_ | VEINTSNPTHRSGESSPVRGDMLGLKSELEKRFFGKTFDDNIHIQLIYNILDIEKILAVYVTNIVYALNN |
| 003462525.1_ | MLGVKGSESHDDFIGYLSTNNTYDVFIDPDNSSLSDDKKANVRKSLSKFNVLLKTKRLGYFGLEEPK |
| ASM346252v1_ | TKDNRVSEAYKKRVYHMLAIVGQIRQCVFHDKSGAKRFDLYSFINNIDPEYRDTLDYLVEERLKSIN |
| genomic | KDFIQGNKVNISLLIDMMKGYEADDIIRLYYDFIVLKSQKNLGFSIKKLREKMLEEYGFRFKDKQYDS |
| SEQ ID NO: | VRSKMYKLMDFLLFCNYYRNDIAAGEALVRKLRFSMTDDEKEGLYADEAAKLWGKFRNDFENIAD |
| 4957 | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKWMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OZGW01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4958 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVTESSRFVYLIKYANAQKIREVAENEKWMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OZHL01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4959 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OYWH01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4960 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| UAAZ01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4961 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OVGV01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4962 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OWSJ01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4963 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKWMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OWGA01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4964 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OVXU01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4965 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OYFN01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4966 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| UPPI01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4967 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| ODUP01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4968 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| ODLK01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4969 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKWMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OOBP01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4970 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OZSD01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4971 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| IMG_ | VEINTSNPTHRSGESSPVRGDMLGLKSELEKRFFGKTFDDNIHIQLIYNILDIEKILAVYVTNIVYALNN |
| 3300009343 | MLGVKGSESHDDFIGYLSTNNTYDVFIDPDNSSLSDDKKANVRKSLSKFNVLLKTKRLGYFGLEEPK |
| SEQ ID NO: | TKDNRVSEAYKKRVYHMLAIVGQIRQCVFHDKSGAKRFDLYSFINNIDPEYRDTLDYLVEERLKSIN |
| 4972 | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| IMG_ | VEINTSNPTHRSGESSPVRGDMLGLKSELEKRFFGKTFDDNIHIQLIYNILDIEKILAVYVTNIVYALNN |
| 3300014803 | MLGVKGSESHDDFIGYLSTNNTYDVFIDPDNSSLSDDKKANVRKSLSKFNVLLKTKRLGYFGLEEPK |
| SEQ ID NO: | TKDNRVSEAYKKRVYHMLAIVGQIRQCVFHDKSGAKRFDLYSFINNIDPEYRDTLDYLVEERLKSIN |
| 4973 | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| UADO01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4974 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKWMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OJQV01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4975 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| UANS01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4976 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| ORRF01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4977 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| UAOB01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4978 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| UADD01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4979 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OZOE01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4980 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVTESSRFVYLIKYANAQKIREVAENEKWMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| UYAJ01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4981 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OYBP01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4982 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OGKO01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4983 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OHAT01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4984 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OWQZ01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4985 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKWMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OXXL01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4986 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| UZSO01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4987 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| UAMJ01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4988 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| UEOP01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4989 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKVVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OGZO01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4990 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKVVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OXOQ01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4991 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKWMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLAIEQLFDRNEYLTEK |
| OPCE01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4992 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKRENDTKQ |
| | EEKIKYEDDLLKNHGYTKDFVKALNSPFGYNIPRFKNLSIEQLFDRNEYLTEK |
| OPCP01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4993 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKRENDTKQ |
| | EEKIKYEDDLLKNHGYTKDFVKALNSPFGYNIPRFKNLSIEQLFDRNEYLTEK |
| UMJY01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4994 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSNMTRKYRNCIAHLTVVRELKEYIGDIRTVDSYFSIYHYVMQRCITKRENDTKQ |
| | EEKIKYEDDLLKNHGYTKDFVKALNSPFGYNIPRFKNLSIEQLFDRNEYLTEK |
| OGZX01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4995 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFYIKKLREKMLEEYGFRFKDKQYD |
| | SVRSKMYKLMDFLLFCNYYRNDIAAGEALVRKLRFSMTDDEKEGLYADEAAKLWGKFRNDFENIA |
| | DHMNGDVTKELGKADMDFDEKILDSEKKNASDLLYFSKMIYMLTYFLDGKEINDLLTTLISKFDNIK |
| | EFLKIMKSSAVNVECELTAGYKLFNDSQRITNELFIVKNIASMRKPAASAKLTMFRDALTILGIDDKIT |
| | DDRISEILKLKEKGKGIHGLRNFITNNVIESSRFVYLIKYANAQKIREVAENEKVVMFVLGGIPDTQIE |
| | RYYKSCVEFPDMNSSLEAKRSELARMIKNIRFDDFKNVKQQAKGRENVAKERAKAVIGLYLTVMYL |
| | LVKNLVNVNARYVIAIHCLERDFGLYKEIIPELASKNLKNDYRILSQTLCELCDDRDESPNLFLKKNK |
| | RLRKCVEVDINNADSNMTRKYRNCIAHLTVVRELKEYIGDIRTVDSYFSIYHYVMQRCITKREDDTK |
| | QEEKIKYEDDLLKNHGYTKDFVKALNSPFGYNIPRFKNLSIEQLFDRNEYLTEK |
| ULZM01.1 | VLSGIFVNAFSSKHGFESGVEINTSNPTHRSGESSPVRGDMLGLKSELEKRFFGKTFDDNIHIQLIYNIL |
| SEQ ID NO: | DIEKILAVYVTNIVYALNNMLGVKGSESHDDFIGYLSTNNTYDVFIDPDNSSLSDDKKANVRKSLSKF |
| 4996 | NVLLKTKRLGYFGLEEPKTKDNRVSEAYKKRVYHMLAIVGQIRQCVFHDKSGAKRFDLYSFINNIDP |
| | EYRDTLDYLVEERLKSINKDFIQGNKVNISLLIDMMKGYEADDIIRLYYDFIVLKSQKNLGFSIKKLRE |
| | KMLEEYGFRFKDKQYDSVRSKMYKLMDFLLFCNYYRNDIAAGEALVRKLRFSMTDDEKEGLYADE |
| | AAKLWGKFRNDFENIADHMNGDVIKELGKADMDFDEKILDSEKKNASDLLYFSKMIYMLTYFLDG |
| | KEINDLLTTLISKFDNIKEFLKIMKSSAVNVECELTAGYKLFNDSQRITNELFIVKNIASMRKPAASAK |
| | LTMFRDALTILGIDDKITDDRISEILKLKEKGKGIHGLRNFITNNVIESSRFVYLIKYANAQKIREVAEN |
| | EKVVMFVLGGIPDTQIERYYKSCVEFPDMNSSLEAKRSELARMIKNIRFDDFKNVKQQAKGRENVA |
| | KERAKAVIGLYLTVMYLLVKNLVNVNARYVIAIHCLERDFGLYKEIIPELASKNLKNDYRILSQTLCE |
| | LCDDRDESPNLFLKKNKRLRKCVEVDINNADSNMTRKYRNCIAHLTVVRELKEYIGDIRTVDSYFSI |
| | YHYVMQRCITKREDDTKQEEKIKYEDDLLKNHGYTKDFVKALNSPFGYNIPRFKNLSIEQLFDRNEY |
| | LTEK |
| GCA_ | MLGLKSELEKRFFGKTFDDNIHIQLIYNILDIEKILAVYVTNIVYALNNMLGVKGSESHDDFIGYLSTN |
| 003460925.1_ | NTYDVFIDPDNSSLSDDKKANVRKSLSKFNVLLKTKRLGYFGLEEPKTKDNRVSEAYKKRVYHMLA |
| ASM346092v1_ | IVGQIRQCVFHDKSGAKRFDLYSFINNIDPEYRDTLDYLVEERLKSINKDFIQGNKVNISLLIDMMKG |
| genomic | YEADDIIRLYYDFIVLKSQKNLGFSIKKLREKMLEEYGFRFKDKQYDSVRSKMYKLMDFLLFCNYYR |
| SEQ ID NO: | NDIAAGEALVRKLRFSMTDDEKEGLYADEAAKLWGKFRNDFENIADHMNGDVIKELGKADMDFDE |
| 4997 | KILDSEKKNASDLLYFSKMIYMLTYFLDGKEINDLLTTLISKFDNIKEFLKIMKSSAVNVECELTAGY |
| | KLFNDSQRITNELFIVKNIASMRKPAASAKLTMFRDALTILGIDDKITDDRISEILKLKEKGKGIHGLR |
| | NFITNNVIESSRFVYLIKYANAQKIREVAENEKWMFVLGGIPDTQIERYYKSCVEFPDMNSSLEAKR |
| | SELARMIKNIRFDDFKNVKQQAKGRENVAKERAKAVIGLYLTVMYLLVKNLVNVNARYVIAIHCLE |
| | RDFGLYKEIIPELASKNLKNDYRILSQTLCELCDDRDESPNLFLKKNKRLRKCVEVDINNADSNMTR |
| | KYRNCIAHLTVVRELKEYIGDIRTVDSYFSIYHYVMQRCITKREDDTKQEEKIKYEDDLLKNHGYTK |
| | DFVKALNSPFGYNIPRFKNLSIEQLFDRNEYLTEK |
| OHAF01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4998 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGIYADEAAKLWGKFRNDFENIAD |
| | HMNGDAIKELGKADMDFDEKILDSEKKNASDLLYFSKMIYMLTYFLDGKEINDLLTTLISKFDNIKEF |
| | LKIMKSSAVDVECELTAGYKLFNDSQRITNELFIVKNIASMRKPAASAKLTMFRDALTILGIDDNITD |
| | DRISEILKLKEKGKGIHGLRNFITNNVTESSRFVYLIKYANAQKIREVAKNEKWMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDKSPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OKSA01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 4999 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGIYADEAAKLWGKFRNDFENIAD |
| | HMNGDAIKELGKADMDFDEKILDSEKKNASDLLYFSKMIYMLTYFLDGKEINDLLTTLISKFDNIKEF |
| | LKIMKSSAVDVECELTAGYKLFNDSQRITNELFIVKNIASMRKPAASAKLTMFRDALTILGIDDNITD |
| | DRISEILKLKEKGKGIHGLRNFITNNVIESSRFVYLIKYANAQKIREVAKNEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDKSPNLFLKKNKR |
| | LRKCVEVDINNADSNMTRKYRNCIAHLTVVRELKEYIGDIRTVDSYFSIYHYVMQRCITKREDDTKQ |
| | EEKIKYEDDLLKNHGYTKDFVKALNSPFGYNIPRFKNLSIEQLFDRNEYLTEK |
| OLVV01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNIYDVFIDPDNSSLSDDKKANVRKSLSKFNVLLKTKRLGYFGLEEPK |
| 5000 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDVVAGEALVRKLRFSMTDDEKEGIYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVDVECELTAGYKLFNDSQRITNELFIVKNIASMRKPAASAKLTMFRDALTILGIDDNITD |
| | DRISEILKLKEKGKGIHGLRNFITNNVIESSRFVYLIKYANAQKIREVAKNEKVVMFVLGGIPDTQIER |
| | YYKSCVEFPDMNSSLEAKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNKR |
| | LRKCVEVDINNADSSMTRKYRNCIAHLTVVRELKEYIGDIRTVDSYFSIYHYVMQRCITKRENDTKQ |
| | EEKIKYEDDLLKNHGYTKDFVKALNSPFGYNIPRFKNLSIEQLFDRNEYLTEK |
| OWZP01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKENIRKSLSKFNALLKTKRLGYFGLEEPKT |
| 5001 | KDTRASEAYKKRVYHMLAIVGQIRQCVFHDKSGAKRFDLYSFINNIDPEYRDTLDYLVEERLKSINK |
| | DFIQGNKVNISLLIDMMKGYEADDIIRLYYDFIVLKSQKNLGFSIKKLREKMLEEYGFRFKDKQYDSV |
| | RSKMYKLMDFLLFCNYYRNDVVAGEALVRKLRFSMTDDEKEGIYADEAAKLWGKFRNDFENIADH |
| | MNGDVTKELGKADMDFDEKILDSEKKNASDLLYFSKMIYMLTYFLDGKEINDLLTTLISKFDNIKEFL |
| | KIMKSSAVNVECELTAGYKLFNDSQRITNELFIVKNIASMRKPAASAKLTMFRDALTILGIDDKITDD |
| | RISEILKLKEKGKGIHGLRNFITNNVTESSRFVYLIKYANAQKIREVAKNEKWMFVLGGIPDTQIERY |
| | YKSCVEFPDMNSSLEAKRSELARMIKNIRFDDFKNVKQQAKGRENVAKERAKAVIGLYLTVMYLLV |
| | KNLVNVNARYVIAIHCLERDFGLYKEIIPELASKNLKNDYRILSQTLCELCDDRDESPNLFLKKNRRL |
| | RKCVEVDINNADSSMTRKYRNCIAHITVVRELKEYIGDIRTVDSYFSIYHYVMQRCITKREDDTKQEE |
| | KIKYEDDLLKNHGYTKDFVKALNSPFGYNIPRFKNLSIEQLFDRNEYLTEK |
| USYS01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5002 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDNITD |
| | DRISEILKLKEKGKGIHGLRNFITNNVIESSRFVYLIKYANAQKIREVAENEKWMFVLGGIPDTQIER |
| | YYKSCVEVPDMNSSLEAKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDERDKSPNLFLKKNER |
| | LRKCVEVGINNADSIMTRKYRNCIAHLTVVRELKEYIGDIRTVDSYFSIYHYVMQRCITKREDDTKQE |
| | EKIKYEDDLLKNHGYTKDFVKALNSPFGYNIPRFKNLSIEQLFDRNEYLTEK |
| OVZZ01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5003 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDNITD |
| | DRISEILKLKEKGKGIHGLRNFITNNVIESSRFVYLIKYANAQKIREVAENEKVVMFVLGGIPDTQIER |
| | YYKSCVEVPDMNSSLEAKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDERDKSPNLFLKKNER |
| | LRKCVEVGINNADSIMTRKYRNCIAHLTVVRELKEYIGDIRTVDSYFSIYHYVMQRCITKREDDTKQE |
| | EKIKYEDDLLKNHGYTKDFVKALNSPFGYNIPRFKNLSIEQLFDRNEYLTEK |
| OLIW01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5004 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDNITD |
| | DRISEILKLKEKGKGIHGLRNFITNNVIESSRFVYLIKYANAQKIREVAENEKVVMFVLGGIPDTQIER |
| | YYKSCVEVPDMNSSLEAKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDERDKSPNLFLKKNER |
| | LRKCVEVGINNADSIMTRKYRNCIAHLTVVRELKEYIGDIRTVDSYFSIYHYVMQRCITKREDDTKQE |
| | EKIKYEDDLLKNHGYTKDFVKALNSPFGYNIPRFKNLSIEQLFDRNEYLTEK |
| OJMH01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5005 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDNITD |
| | DRISEILKLKEKGKGIHGLRNFITNNVIESSRFVYLIKYANAQKIREVAENEKVVMFVLGGIPDTQIER |
| | YYKSCVEVPDMNSSLEAKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDERDKSPNLFLKKNER |
| | LRKCVEVGINNADSIMTRKYRNCIAHLTVVRELKEYIGDIRTVDSYFSIYHYVMQRCITKREDDTKQE |
| | EKIKYEDDLLKNHGYTKDFVKALNSPFGYNIPRFKNLSIEQLFDRNEYLTEK |
| OVFR01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5006 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDNITD |
| | DRISEILKLKEKGKGIHGLRNFITNNVIESSRFVYLIKYANAQKIREVAENEKVVMFVLGGIPDTQIER |
| | YYKSCVEVPDMNSSLEAKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDERDKSPNLFLKKNER |
| | LRKCVEVGINNADSIMTRKYRNCIAHLTVVRELKEYIGDIRTVDSYFSIYHYVMQRCITKREDDTKQE |
| | EKIKYEDDLLKNHGYTKDFVKALNSPFGYNIPRFKNLSIEQLFDRNEYLTEK |
| OOBT01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5007 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDNITD |
| | DRISEILKLKEKGKGIHGLRNFITNNVIESSRFVYLIKYANAQKIREVAENEKWMFVLGGIPDTQIER |
| | YYKSCVEVPDMNSSLEAKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDERDKSPNLFLKKNER |
| | LRKCVEVGINNADSIMTRKYRNCIAHLTVVRELKEYIGDIRTVDSYFSIYHYVMQRCITKREDDTKQE |
| | EKIKYEDDLLKNHGYTKDFVKALNSPFGYNIPRFKNLSIEQLFDRNEYLTEK |
| OZPH01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5008 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVTESSRFVYLIKYANAQKIRKVAENEKWMFVLGGIPDTQIER |
| | YYKSCVEFPDMNSSLEVKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| OGPN01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5009 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIRKVAENEKVVMFVLGGIPDTQIER |
| | YYKSCVEFPDMNSSLEVKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| OLXN01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5010 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIRKVAENEKVVMFVLGGIPDTQIER |
| | YYKSCVEFPDMNSSLEVKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| OLWT01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5011 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIRKVAENEKVVMFVLGGIPDTQIER |
| | YYKSCVEFPDMNSSLEVKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| IMG_ | VEINTSNPTHRSGESSPVRGDMLGLKSELEKRFFGKTFDDNIHIQLIYNILDIEKILAVYVTNIVYALNN |
| 3300007501 | MLGVKGSESHDDFIGYLSTNNTYDVFIDPDNSSLSDDKKANVRKSLSKFNVLLKTKRLGYFGLEEPK |
| SEQ ID NO: | TKDNRVSEAYKKRVYHMLAIVGQIRQCVFHDKSGAKRFDLYSFINNIDPEYRDTLDYLVEERLKSIN |
| 5012 | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIRKVAENEKVVMFVLGGIPDTQIER |
| | YYKSCVEFPDMNSSLEVKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| OWFT01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5013 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIRKVAENEKWMFVLGGIPDTQIER |
| | YYKSCVEFPDMNSSLEVKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| IMG_ | VEINTSNPTHRSGESSPVRGDMLGLKSELEKRFFGKTFDDNIHIQLIYNILDIEKILAVYVTNIVYALNN |
| 3300008260 | MLGVKGSESHDDFIGYLSTNNTYDVFIDPDNSSLSDDKKANVRKSLSKFNVLLKTKRLGYFGLEEPK |
| SEQ ID NO: | TKDNRVSEAYKKRVYHMLAIVGQIRQCVFHDKSGAKRFDLYSFINNIDPEYRDTLDYLVEERLKSIN |
| 5014 | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIRKVAENEKVVMFVLGGIPDTQIER |
| | YYKSCVEFPDMNSSLEVKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| IMG_ | VEINTSNPTHRSGESSPVRGDMLGLKSELEKRFFGKTFDDNIHIQLIYNILDIEKILAVYVTNIVYALNN |
| 3300007717 | MLGVKGSESHDDFIGYLSTNNTYDVFIDPDNSSLSDDKKANVRKSLSKFNVLLKTKRLGYFGLEEPK |
| SEQ ID NO: | TKDNRVSEAYKKRVYHMLAIVGQIRQCVFHDKSGAKRFDLYSFINNIDPEYRDTLDYLVEERLKSIN |
| 5015 | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIRKVAENEKVVMFVLGGIPDTQIER |
| | YYKSCVEFPDMNSSLEVKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| OYAL01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5016 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIRKVAENEKVVMFVLGGIPDTQIER |
| | YYKSCVEFPDMNSSLEVKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| UBIW01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5017 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIRKVAENEKVVMFVLGGIPDTQIER |
| | YYKSCVEFPDMNSSLEVKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| OYBU01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5018 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIRKVAENEKVVMFVLGGIPDTQIER |
| | YYKSCVEFPDMNSSLEVKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| OZLE01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5019 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVTESSRFVYLIKYANAQKIRKVAENEKWMFVLGGIPDTQIER |
| | YYKSCVEFPDMNSSLEVKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| OYDM01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5020 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIRKVAENEKVVMFVLGGIPDTQIER |
| | YYKSCVEFPDMNSSLEVKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| UAYF01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5021 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIRKVAENEKVVMFVLGGIPDTQIER |
| | YYKSCVEFPDMNSSLEVKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| UXNO01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5022 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIRKVAENEKVVMFVLGGIPDTQIER |
| | YYKSCVEFPDMNSSLEVKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| OPFT01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5023 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIRKVAENEKVVMFVLGGIPDTQIER |
| | YYKSCVEFPDMNSSLEVKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| UAIT01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5024 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIRKVAENEKWMFVLAGIPDTQIER |
| | YYKSCVEFPDMNSSLEVKRSELARMIKNISFDDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| OZBH01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5025 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIRKVAENEKVVMFVLAGIPDTQIER |
| | YYKSCVEFPDMNSSLEVKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| UMGG01.1 | MMKGYEADDIIRLYYDFIVLKSQKNLGFSIKKLREKMLEEYGFRFKDKQYDSVRSKMYKLMDFLLF |
| SEQ ID NO: | CNYYRNDIAAGEALVRKLRFSMTDDEKEGLYADEAAKLWGKFRNDFENIADHMNGDVTKELGKAD |
| 5026 | MDFDEKILDSEKKNASDLLYFSKMIYMLTYFLDGKEINDLLTTLISKFDNIKEFLKIMKSSAVNVECE |
| | LTAGYKLFNDSQRITNELFIVKNIASMRKPAASAKLTMFRDALTILGIDDKITDDRISEILKLKEKGKGI |
| | HGLRNFITNNVTESSRFVYLIKYANAQKIRKVAENEKWMFVLGGIPDTQIERYYKSCVEFPDMNSSL |
| | EVKRSELARMIKNISFDDFKNVKQQAKGRENVAKERAKAVIGLYLTVMYLLVKNLVNVNARYVIAI |
| | HCLERDFGLYKEIIPELASKNLKNDYRILSQTLCELCDKSPNLFLKKNERLRKCVEVDINNADSSMTR |
| | KYRNCIAHLTVVRELKEYIGDIRTVDSYFSIYHYVMQRCITKREDDKKQEEKIKFEDDLLKNHGYTK |
| | DFVKALNSPFGYNIPRFKNLSIEQLFDRNEYLTEK |
| OZVN01.1 | MMKGYEADDIIRLYYDFIVLKSQKNLGFSIKKLREKMLEEYGFRFKDKQYDSVRSKMYKLMDFLLF |
| SEQ ID NO: | CNYYRNDIAAGEALVRKLRFSMTDDEKEGLYADEAAKLWGKFRNDFENIADHMNGDVTKELGKAD |
| 5027 | MDFDEKILDSEKKNASDLLYFSKMIYMLTYFLDGKEINDLLTTLISKFDNIKEFLKIMKSSAVNVECE |
| | LTAGYKLFNDSQRITNELFIVKNIASMRKPAASAKLTMFRDALTILGIDDKITDDRISEILKLKEKGKGI |
| | HGLRNFITNNVTESSRFVYLIKYANAQKIRKVAENEKWMFVLGGIPDTQIERYYKSCVEFPDMNSSL |
| | EVKRSELARMIKNISFDDFKNVKQQAKGRENVAKERAKAVIGLYLTVMYLLVKNLVNVNARYVIAI |
| | HCLERDFGLYKEIIPELASKNLKNDYRILSQTLCELCDKSPNLFLKKNERLRKCVEVDINNADSSMTR |
| | KYRNCIAHLTVVRELKEYIGDIRTVDSYFSIYHYVMQRCITKREDDKKQEEKIKFEDDLLKNHGYTK |
| | DFVKALNSPFGYNIPRFKNLSIEQLFDRNEYLTEK |
| ORTF01.1 | MMKGYEADDIIRLYYDFIVLKSQKNLGFSIKKLREKMLEEYGFRFKDKQYDSVRSKMYKLMDFLLF |
| SEQ ID NO: | CNYYRNDIAAGEALVRKLRFSMTDDEKEGLYADEAAKLWGKFRNDFENIADHMNGDVIKELGKAD |
| 5028 | MDFDEKILDSEKKNASDLLYFSKMIYMLTYFLDGKEINDLLTTLISKFDNIKEFLKIMKSSAVNVECE |
| | LTAGYKLFNDSQRITNELFIVKNIASMRKPAASAKLTMFRDALTILGIDDKITDDRISEILKLKEKGKGI |
| | HGLRNFITNNVTESSRFVYLIKYANAQKIRKVAENEKWMFVLGGIPDTQIERYYKSCVEFPDMNSSL |
| | EVKRSELARMIKNISFDDFKNVKQQAKGRENVAKERAKAVIGLYLTVMYLLVKNLVNVNARYVIAI |
| | HCLERDFGLYKEIIPELASKNLKNDYRILSQTLCELCDKSPNLFLKKNERLRKCVEVDINNADSSMTR |
| | KYRNCIAHLTVVRELKEYIGDIRTVDSYFSIYHYVMQRCITKREDDKKQEEKIKFEDDLLKNHGYTK |
| | DFVKALNSPFGYNIPRFKNLSIEQLFDRNEYLTEK |
| UAOK01.1 | MMKGYEADDIIRLYYDFIVLKSQKNLGFSIKKLREKMLEEYGFRFKDKQYDSVRSKMYKLMDFLLF |
| SEQ ID NO: | CNYYRNDIAAGEALVRKLRFSMTDDEKEGLYADEAAKLWGKFRNDFENIADHMNGDVTKELGKAD |
| 5029 | MDFDEKILDSEKKNASDLLYFSKMIYMLTYFLDGKEINDLLTTLISKFDNIKEFLKIMKSSAVNVECE |
| | LTAGYKLFNDSQRITNELFIVKNIASMRKPAASAKLTMFRDALTILGIDDKITDDRISEILKLKEKGKGI |
| | HGLRNFITNNVTESSRFVYLIKYANAQKIRKVAENEKWMFVLGGIPDTQIERYYKSCVEFPDMNSSL |
| | EVKRSELARMIKNISFDDFKNVKQQAKGRENVAKERAKAVIGLYLTVMYLLVKNLVNVNARYVIAI |
| | HCLERDFGLYKEIIPELASKNLKNDYRILSQTLCELCDKSPNLFLKKNERLRKCVEVDINNADSSMTR |
| | KYRNCIAHLTVVRELKEYIGDIRTVDSYFSIYHYVMQRCITKREDDKKQEEKIKFEDDLLKNHGYTK |
| | DFVKALNSPFGYNIPRFKNLSIEQLFDRNEYLTEK |
| UAOC01.1 | MMKGYEADDIIRLYYDFIVLKSQKNLGFSIKKLREKMLEEYGFRFKDKQYDSVRSKMYKLMDFLLF |
| SEQ ID NO: | CNYYRNDIAAGEALVRKLRFSMTDDEKEGLYADEAAKLWGKFRNDFENIADHMNGDVTKELGKAD |
| 5030 | MDFDEKILDSEKKNASDLLYFSKMIYMLTYFLDGKEINDLLTTLISKFDNIKEFLKIMKSSAVNVECE |
| | LTAGYKLFNDSQRITNELFIVKNIASMRKPAASAKLTMFRDALTILGIDDKITDDRISEILKLKEKGKGI |
| | HGLRNFITNNVTESSRFVYLIKYANAQKIRKVAENEKWMFVLGGIPDTQIERYYKSCVEFPDMNSSL |
| | EVKRSELARMIKNISFDDFKNVKQQAKGRENVAKERAKAVIGLYLTVMYLLVKNLVNVNARYVIAI |
| | HCLERDFGLYKEIIPELASKNLKNDYRILSQTLCELCDKSPNLFLKKNERLRKCVEVDINNADSSMTR |
| | KYRNCIAHLTVVRELKEYIGDIRTVDSYFSIYHYVMQRCITKREDDKKQEEKIKFEDDLLKNHGYTK |
| | DFVKALNSPFGYNIPRFKNLSIEQLFDRNEYLTEK |
| OZVA01.1 | MMKGYEADDIIRLYYDFIVLKSQKNLGFSIKKLREKMLEEYGFRFKDKQYDSVRSKMYKLMDFLLF |
| SEQ ID NO: | CNYYRNDIAAGEALVRKLRFSMTDDEKEGLYADEAAKLWGKFRNDFENIADHMNGDVTKELGKAD |
| 5031 | MDFDEKILDSEKKNASDLLYFSKMIYMLTYFLDGKEINDLLTTLISKFDNIKEFLKIMKSSAVNVECE |
| | LTAGYKLFNDSQRITNELFIVKNIASMRKPAASAKLTMFRDALTILGIDDKITDDRISEILKLKEKGKGI |
| | HGLRNFITNNVTESSRFVYLIKYANAQKIRKVAENEKVVMFVLGGIPDTQIERYYKSCVEFPDMNSSL |
| | EVKRSELARMIKNISFDDFKNVKQQAKGRENVAKERAKAVIGLYLTVMYLLVKNLVNVNARYVIAI |
| | HCLERDFGLYKEIIPELASKNLKNDYRILSQTLCELCDKSPNLFLKKNERLRKCVEVDINNADSSMTR |
| | KYRNCIAHLTVVRELKEYIGDIRTVDSYFSIYHYVMQRCITKREDDKKQEEKIKFEDDLLKNHGYTK |
| | DFVKALNSPFGYNIPRFKNLSIEQLFDRNEYLTEK |
| OZEB01.1_2 | MNGDVTKELGKADMDFDEKILDSEKKNASDLLYFSKMIYMLTYFLDGKEINDLLTTLISKFDNIKEFL |
| SEQ ID NO: | KIMKSSAVNVECELTAGYKLFNDSQRITNELFIVKNIASMRKPAASAKLTMFRDALTILGIDDKITDD |
| 5032 | RISEILKLKEKGKGIHGLRNFITNNVIESSRFVYLIKYANAQKIRKVAENEKVVMFVLGGIPDTQIERY |
| | YKSCVEFPDMNSSLEVKRSELARMIKNISFDDFKNVKQQAKGRENVAKERAKAVIGLYLTVMYLLV |
| | KNLVNVNARYVIAIHCLERDFGLYKEIIPELASKNLKNDYRILSQTLCELCDKSPNLFLKKNERLRKC |
| | VEVDINNADSSMTRKYRNCIAHLTVVRELKEYIGDIRTVDSYFSIYHYVMQRCITKREDDKKQEEKI |
| | KFEDDLLKNHGYTKDFVKALNSPFGYNIPRFKNLSIEQLFDRNEYLTEK |
| UPFF01.1 | MDFLLFCNYYRNDVIAGEALVRKLRFSMTDDEKEGIYADEAAKLWGKFRNDFENIADHMNGDVIK |
| SEQ ID NO: | ELGKADMDFDEKILDSEKKNASDLLYFSKMIYMLTYFLDGKEINDLLTTLISKFDNIKEFLKIMKSSA |
| 5033 | VNVECELTAGYKLFNDSQRITNELFIVKNIASMRKPAASAKLTMFRDALTILGIDDNITDDRISEILKL |
| | KEKGKGIHGLRNFITNNVIESSRFVYLIKYANAQKIRKVAENEKVVMFVLGGIPDTQIERYYKSCVEF |
| | PDMNSSLEAKRSELARMIKNIRFDDFKNVKQQAKGRENVAKERAKAVIGLYLTVMYLLVKNLVNV |
| | NARYVIAIHCLERDFGLYKEIIPELASKNLKNDYRILSQTLCELCDDRDESPNLFLKKNKRLRKCVEV |
| | DINNADSSMTRKYRNCIAHLTVVRELKEYIGDIRTVDSYFSIYHYVMQRCITKREDDKKQEEKIKYE |
| | DDLLKNHGYTKDFVKALNSPFGYNIPRFKNLSIEQLFDRNEYLTEK |
| OZAU01.1 | VLHFGVGIYADEAAKLWGKFRNDFENIADHMNGDVIKELGKADMDFDEKILDSEKKNASDLLYFSK |
| SEQ ID NO: | MIYMLTYFLDGKEINDLLTTLISKFDNIKEFLKIMKSSAVDVECELTAGYKLFNDSQRITNELFIVKNI |
| 5034 | ASMRKPAASAKLTMFRDALTILGIDDNITDDRISEILKLKEKGKGIHGLRNFITNNVIESSRFVYLIKYA |
| | NAQKIREVAKNEKVVMFVLGGIPDTQIERYYKSCVEFPDMNSSLEAKRSELARMIKNISFDDFKNVK |
| | QQAKGRENVAKERAKAVIGLYLTVMYLLVKNLVNVNARYVIAIHCLERDFGLYKEIIPELASKNLK |
| | NDYRILSQTLCELCDDRDESPNLFLKKNKRLRKCVEVDINNADSSMTRKYRNCIAHLTVVRELKEYI |
| | GDIRTVDSYFSIYHYVMQRCITKRENDTKQEEKIKYEDDLLKNHGYTKDFVKALNSPFGYNIPRFKN |
| | LSIEQLFDRNEYLTEK |
| UPRH01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5035 | TKDTRVSQAYKKRVYHMLAIVGQIRQSVFHDKSSKLHEDLYSFIDIIDSEYRETLDYLVEERLKSINK |
| | DFIEGNKVNISLLIDMMKGYEADDIIRLYYDFIVLKSQKNLGFSIKKLREKMLDEYGFRFKDKQYDSV |
| | RSKMYKLMDFLLFCNYYRNDVVAGEALVRKLRFSMTDDEKEGIYADEAAKLWGKFRNDFENIADH |
| | MNGDVTKELGKADMDFDEKILDSEKKNASDLLYFSKMIYMLTYFLDGKEINDLLTTLISKFDNIKEFL |
| | KIMKSSAVDVECELTAGYKLFNDSQRITNELFIVKNIASMRKPAASAKLTMFRDALTILGIDDNITDD |
| | RISEILKLKEKGKGIHGLRNFITNNVIESSRFVYLIKYANAQKIREVAKNEKVVMFVLGGIPDTQIERY |
| | YKSCVEFPDMNSSLEAKRSELARMIKNISFDDFKNVKQQAKGRENVAKERAKAVIGLYLTVMYLLV |
| | KNLVNVNARYVIAIHCLERDFGLYKEIIPELASKNLKNDYRILSQTLCELCDDRDESPNLFLKKNKRL |
| | RKCVEVDINNADSSMTRKYRNCIAHLTVVRELKEYIGDIRTVDSYFSIYHYVMQRCITKRENDTKQE |
| | EKIKYEDDLLKNHGYTKDFVKALNSPFGYNIPRFKNLSIEQLFDRNEYLTEK |
| UPRY01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5036 | TKDTRVSQAYKKRVYHMLAIVGQIRQSVFHDKSSKLHEDLYSFIDIIDSEYRETLDYLVEERLKSINK |
| | DFIEGNKVNISLLIDMMKGYEADDIIRLYYDFIVLKSQKNLGFSIKKLREKMLDEYGFRFKDKQYDSV |
| | RSKMYKLMDFLLFCNYYRNDVVAGEALVRKLRFSMTDDEKEGIYADEAAKLWGKFRNDFENIADH |
| | MNGDVTKELGKADMDFDEKILDSEKKNASDLLYFSKMIYMLTYFLDGKEINDLLTTLISKFDNIKEFL |
| | KIMKSSAVDVECELTAGYKLFNDSQRITNELFIVKNIASMRKPAASAKLTMFRDALTILGIDDNITDD |
| | RISEILKLKEKGKGIHGLRNFITNNVIESSRFVYLIKYANAQKIREVAKNEKVVMFVLGGIPDTQIERY |
| | YKSCVEFPDMNSSLEAKRSELARMIKNISFDDFKNVKQQAKGRENVAKERAKAVIGLYLTVMYLLV |
| | KNLVNVNARYVIAIHCLERDFGLYKEIIPELASKNLKNDYRILSQTLCELCDDRDESPNLFLKKNKRL |
| | RKCVEVDINNADSSMTRKYRNCIAHLTVVRELKEYIGDIRTVDSYFSIYHYVMQRCITKRENDTKQE |
| | EKIKYEDDLLKNHGYTKDFVKALNSPFGYNIPRFKNLSIEQLFDRNEYLTEK |
| UPDQ01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5037 | TKDTRVSQAYKKRVYHMLAIVGQIRQSVFHDKSSKLHEDLYSFIDIIDSEYRETLDYLVEERLKSINK |
| | DFIEGNKVNISLLIDMMKGYEADDIIRLYYDFIVLKSQKNLGFSIKKLREKMLDEYGFRFKDKQYDSV |
| | RSKMYKLMDFLLFCNYYRNDVVAGEALVRKLRFSMTDDEKEGIYADEAAKLWGKFRNDFENIADH |
| | MNGDVTKELGKADMDFDEKILDSEKKNASDLLYFSKMIYMLTYFLDGKEINDLLTTLISKFDNIKEFL |
| | KIMKSSAVDVECELTAGYKLFNDSQRITNELFIVKNIASMRKPAASAKLTMFRDALTILGIDDNITDD |
| | RISEILKLKEKGKGIHGLRNFITNNVIESSRFVYLIKYANAQKIREVAKNEKVVMFVLGGIPDTQIERY |
| | YKSCVEFPDMNSSLEAKRSELARMIKNISFDDFKNVKQQAKGRENVAKERAKAVIGLYLTVMYLLV |
| | KNLVNVNARYVIAIHCLERDFGLYKEIIPELASKNLKNDYRILSQTLCELCDDRDESPNLFLKKNKRL |
| | RKCVEVDINNADSSMTRKYRNCIAHLTVVRELKEYIGDIRTVDSYFSIYHYVMQRCITKRENDTKQE |
| | EKIKYEDDLLKNHGYTKDFVKALNSPFGYNIPRFKNLSIEQLFDRNEYLTEK |
| OWKP01.1 | MLGLKSELEKRFFGKTFDDNIHIQLIYNILDIEKILAVYITNIVYALNNMLGVKGSESHDDFIGYLSAR |
| SEQ ID NO: | NTYEVFTHPDKSNLSDKVKGNINKVKGNIKKSLSKFNDLLKTKRLGYFGLEEPKTKDKRVSEAYKK |
| 5038 | RVYHMLAIVGQIRQSVFHDKSNELDEYLYSFIDIIDSEYRDTLDYLVDERFDSINKGFVQGNKVNISLL |
| | IDMMKGYEADDIIRLYYDFIVLKSQKNLGFSIKKLREKMLDEYGFRFKDKQYDSVRSKMYKLMDFL |
| | LFCNYYRNDVVAGEVLVRKLRFSMTDDEKEGIYADEAAKLWGKFRNDFENIADHMNGDVIKELGK |
| | ADMDFDEKIIDSEKKNASDLLYFSKMIYMLTYFLDGKEINDLLTTLISKFDNIKEFLKIMKSSAVDVE |
| | CELTAGYKLFNDRQRITNELFIVKNIASMRKPAASAKLTMFRDALTILGIDDNITDDRISEILKLKEKG |
| | KGIHGLRNFITNNVIESSRFVYLIKYANAQKIREVAKNEKWMFVLGGIPDTQIERYYKSCVEFPDMN |
| | SSLEAKRSELARMIKNIRFDDFKNVKQQAKGRENVAKERAKAVIGLYLTVMYLLVKNLVNVNARY |
| | VIAIHCLERDFGLYKEIIPELASKNLKNDYRILSQTLCELCDNGDESPNLFLKKNKRLRKCVEVDINNA |
| | DSNMTRKYRNCIAHLTVVRELKEYIGDIRTVDSYFSIYHYVMQRCITKRENDTKQEEKIKYEDDLLK |
| | NHGYTKDFVKALNSPFGYNIPRFKNLSIEQLFDRNEYLTEK |
| UACT01.1 | VEINTSNPTHRSGESSPVRGDMLGLKSELEKRFFGKTFDDNIHIQLIYNILDIEKILAVYVTNIVYALNN |
| SEQ ID NO: | MLGIKDSESYDDFMGYLSARNTYEVFTHPDKSNLSDKVKGNINKVKGNIKKSLSKFNDLLKTKRLG |
| 5039 | YFGLEEPKTKDKRVSEAYKKRVYHMLAIVGQIRQSVFHDKSNELDEYLYSFIDIIDSEYRDTLDYLVD |
| | ERFDSINKGFVQGNKVNISLLIDMMKGYEADDIIRLYYDFIVLKSQKNLGFSIKKLREKMLDEYGFRF |
| | KDKQYDSVRSKMYKLMDFLLFCNYYRNDVVAGEVLVRKLRFSMTDDEKEGIYADEAAKLWGKFR |
| | NDFENIADHMNGDVIKELGKADMDFDEKIIDSEKKNASDLLYFSKMIYMLTYFLDGKEINDLLTTLIS |
| | KFDNIKEFLKIMKSSAVDVECELTAGYKLFNDSQRITNELFIVKNIASMRKPAASAKLTMFRDALTIL |
| | GIDDNITDDRISEILKLKEKGKGIHGLRNFITNNVIESSRFVYLIKYANAQKIREVAKNEKVVMFVLGG |
| | IPDTQIERYYKSCVEFPDMNSSLEAKRSELARMIKNIRFDDFKNVKQQAKGRENVAKERAKAVIGLY |
| | LTVMYLLVKNLVNVNARYVIAIHCLERDFGLYKEIIPELASKNLKNDYRILSQTLCELCDNGDESPNL |
| | FLKKNKRLRKCVEVDINNADSNMTRKYRNCIAHLTVVRELKEYIGDIRTVDSYFSIYHYVMQRCITK |
| | RENDTKQEEKIKYEDDLLKNHGYTKDFVKALNSPFGYNIPRFKNLSIEQLFDRNEYLTEK |
| OYVL01.1 | VLKSQKNLGFSIKKLREKILDEYGFRFKDKQYDSVRSKMYKLMDFLLFCNYYRNDIAAGESLVRKL |
| SEQ ID NO: | RFSMTDDEKEGIYADEAAKLWGKFRNDFENIADHMNGDVIKELGKADMDFDEKILDSEKKNASDL |
| 5040 | LYFSKMIYMLTYFLDGKEINDLLTTLISKFDNIKEFLKIMKSSAIDVECELTAGYKLFNDSQRITNELFI |
| | VKNIASMRKPAASAKLTMFRDALTILGIDDKITDDRISEILKLKEKGKGIHGLRNFITNNVIESSRFVYL |
| | IKYANAQKIREVAKNEKVVMFVLGGIPDTQIERYYKSCVEFPDMNSSLEAKRSELARMIKNIRFDDF |
| | KNVKQQAKGRENVAKERAKAVIGLYLTVMYLLVKNLVNVNARYVIAIHCLERDFGLYKEIIPELAS |
| | KNLKNDYRILSQTLCELCDNGDESPNLFLKKNKRLRKCVEVDINNADSSMTRKYRNCIAHLTVVREL |
| | KEYIGDIRTVDSYFSIYHYVMQRCITKREDDTKQEEKIKYEDDLLKNHGYTKDFVKALNSPFGYNIPR |
| | FKNLSIEQLFDRNEYLTEK |
| OWXF01.1 | MLTYFLDGKEINDLLTTLISKFDNUCEFLKIMKSSAVDVECELTAGYKLFNDSQRITNELFIVKNIASM |
| SEQ ID NO: | RKPAASAKLTMFRDALTILGIDDKITDDRISEILKLKEKGKGIHGLRNFITNNVIESSRFVYLIKYANAQ |
| 5041 | KIREVAKNEKVVMFVLGGIPDTQIERYYKSCVEFPDMNSSLEAKRSELARMIKNIRFDDFKNVKQQA |
| | KGRENVAKERAKAVIGLYLTVMYLLVKNLVNVNARYVIAIHCLERDFGLYKEIIPELASKNLKNDY |
| | RILSQTLCELCDNGDESPNLFLKKNKRLRKCVEVDINNADSNMTRKYRNCIAHLTVVRELKEYIGDI |
| | RTVDSYFSIYHYVMQRCITKREDDTKQEEKIKYEDDLLKNHGYTKDFVKALNSPFGYNIPRFKNLSIE |
| | QLFDRNEYLTEK |
| OWEF01.1 | MLAIVGQIRQCVFHDKSGAKRFDLYSFINNIYPEYRETLDYLVDERFDSINKGFIQGNKVNISLLIDM |
| SEQ ID NO: | MKGYEADDIIRLYYDFIVLKSQKNLGFSIKKLREKMLDEYGFRFKDKQYDSVRSKMYKLMDFLLFC |
| 5042 | NYYRNDVAAGEALVRKLRFSMTDDEKEGIYAGEAAKLWGKFRNDFENIADHMNGDVIKELGKAD |
| | MDFDEKILDSEKKNASDLLYFSKMrYMLTYFLDGKEINDLLTTLISKFDNIKEFLKIMKSSAVDVECE |
| | LTAGYKLFNDSQRITNELFIVKNIASMRKPAASAKLTMFRDALTILGIDDNITDDRISEILKLKEKGKGI |
| | HGLRNFITNNVTESSRFVYLIKYANAQKIREVAKNEKVVMFVLGGIPDTQIERYYKSCVEVPDMNSSL |
| | EAKRSELARMIKNIRFDDFKNVKQQAKGRENVAKERAKAVIGLYLTVMYLLVKNLVNVNARYVIAI |
| | HCLERDFGLYKEIIPELASKNLKNDYRILSQTLCELCDDRDKSPNLFLKKNKRLRKCVEVDINNADSS |
| | MTRKYRNCIAHLTVVRELKEYIGDIRTVDSYFSIYHYVMQRCITKRENDTKQEDKIKYEDDLLKNHG |
| | YTKDFVKALNSPFGYNIPRFKNLSIEQLFDRNEYLTEK |
| OAUV01.1 | MLAIVGQIRQCVFHDKSGAKRFDLYSFINNIDPEYRETLDYLVDERFDSINKGFIEGNKINISLLIDMM |
| SEQ ID NO: | KGYEADDIIRLYYDFIVLKSQKNLGFSIKKLREKMLDEYGFRFKDKQYDPVRSKMYKLMDFLLFCN |
| 5043 | HYRNDVAAGEALVRKLRFSMTDDEKEGIYADEAAKLWGKFRNDFENIADHMNGDVIKELGKADM |
| | DFDEKILDSEKKNASDLLYFSKMIYMLTYFLDGKEINDLLTTLISKFDNIKEFLKIMKSSAVDVECELT |
| | AGYKLFNDSQRITNELFIVKNIASMRKPAASAKLTMFRDALTILGIDDNITDDRISEILKLKEKGKGIH |
| | GLRNFITNNVIESSRFVYLIKYANAQKIREVAKNEKWMFVLGGIPDTQIERYYKSCVEFPDMNSSLE |
| | AKRSELARMIKNISFDDFKNVKQQAKGRENVAKERAKAVIGLYLTVMYLLVKNLVNVNARYVIAIH |
| | CLERDFGLYKEIIPELASKNLKNDYRILSQTLCELCDDRDESPNLFLKKNKRLRKCVEVDINNADSSM |
| | TRKYRNCIAHLTVVRELKEYIGDIRTVDSYFSIYHYVMQRCITKRENDTKQEEKIKYEDDLLKNHGY |
| | TKDFVKALNSPFGYNIPRFKNLSIEQLFDRNEYLTEK |
| OXCB01.1 | VEINTSNPTHRSGESSPVRWDMLGLKSELEKRFFGKTFDDNIHIQLIYNILDIEKILAVYVTNIVYALN |
| SEQ ID NO: | NMLGEGDDESHDDFMGYLSAQNTYYIFTHPDKSNLSDKVKGNIKKSLSKFNDLLKTKRLGYFGLEE |
| 5044 | PKTKDKRVSEAYKKRVYHMLAIVGQIRQSVFHDKSNELDEYLYSFIDIIDSEYRDTLDYLVDERFDSI |
| | NKGFVQGNKVNISLLIDMMKGYEADDIIRLYYDFIVLKSQKNLGFSIKKLREKMLDEYGFRFKDKQY |
| | DSVRSKMYKLMDFLLFCNYYRNDVVAGEVLVRKLRFSMTDDEKEWIYADEAEKLWGKFRNDFENI |
| | ADHMNGDVTKELGKADMDFDEKILDSEKKNASDLLYFSKMIYMLTYFLDGKEINDLLTTLISKFDNI |
| | KEFLKIMKSSAVDVECELTAGYKLFNDSQRITNELFIVKNIASMRKPAASAKLTMFRDALTILGIDDKI |
| | TDDRISEILKLKEKGKGIHGLRNFITNNVIESSRFVYLIKYANAQKIREVAENEKWMFVLGGIPDTQI |
| | ERYYKSCVEFPDMNSSLEVKRSELARMIKNISFDDFKNVKQQAKGRENVAKERAKAVIGLYLTVMY |
| | LLVKNLVNVNARYVIAIHCLERDFGLYKEIIPELASKNLKNDYRILSQTLCELCDDRDESPNLFLKKN |
| | KRLRKCVEVDINNADSSMTRKYRNCIAHLTVVRELKEYIGDIRTVDSYFSIYHYVMQRCITKREDDK |
| | KQEEKIKYEDDLLKNHGYTKDFVKALNSPFGYNIPRFKNLSIEQLFDRNEYLTEK |
| ORRC01.1 | VEINTSNPTHRSGESSSVRGDMLGLKSELEKRFFGKTFDDNIHIQLIYNILDIEKILAVYVTNIVYALNN |
| SEQ ID NO: | MLGVKGSESYDDFMGYLSAQNTYYIFTHPDKSNLSDKVKGNIKKSLSKFNDLLKTKRLGYFGLEEP |
| 5045 | KTKDKRVSEAYKKRVYHMLAIVGQIRQSVFHDKSNELDEYLYSFIDIIDSEYRDTLDYLVDERFDSIN |
| | KGFVQGNKVNISLLIDMMKGYEADDIIRLYYDFIVLKSQKNLGFSIKKLREKMLDEYGFRFKDKQYD |
| | SVRSKMYKLMDFLLFCNYYRNDVVAGEALVRKLRFSMTDDEKEGIYADEAEKLWGKFRNDFENIA |
| | DHMNGDVTKELGKADMDFDEKILDSEKKNASDLLYFSKMIYMLTYFLDGKEINDLLTTLISKFDNIK |
| | EFLKIMKSSAVDVECELTAGYKLFNDSQRITNELFIVKNIASMRKPASSAKLTMFRDALTILGIDDNIT |
| | DDRISEILKLKEKGKGIHGLRNFITNNVIESSRFVYLIKYANAQKIREVAKNEKWMFVLGGIPDTQIE |
| | RYYKSCVEFPDMNSSMGAKRRELAKMIKSISFEDFKDVKQQAKGRENVAKERAKAVIGLYLTVMY |
| | LLVKNLVNVNARYVIAIHCLERDFGLYKEIIPELASKNLKNDYRILSQTLCELCDNGDESPNLFLKKN |
| | KRLRKCVEVDINNADSNMTRKYRNCIAHLTVVRELNKYIKDIRTVDSYFSIYHYVMQRCITKRENDT |
| | KQEEKINYEDDLLKNHGYTKDFVKALNSPFGYNIPRFKNLSIEQLFDRNEYLTEK |
| GCA_ | VEINTSNPTHRSGESSSVRGDMLGLKSELEKRFFGKTFDDNIHIQLIYNILDIEKILAVYVTNIVYALNN |
| 900066635. | MLGVKGSESYDDFMGYLSAQNTYYIFTHPDKSNLSDKVKGNIKKSLSKFNDLLKTKRLGYFGLEEP |
| 1_14207_7_17_ | KTKDKRVSEAYKKRVYHMLAIVGQIRQSVFHDKSNELDEYLYSFIDIIDSEYRDTLDYLVDERFDSIN |
| genomic | KGFVQGNKVNISLLIDMMKGYEADDIIRLYYDFIVLKSQKNLGFSIKKLREKMLDEYGFRFKDKQYD |
| SEQ ID NO: | SVRSKMYKLMDFLLFCNYYRNDVVAGEALVRKLRFSMTDDEKEGIYADEAEKLWGKFRNDFENIA |
| 5046 | DHMNGDVTKELGKADMDFDEKILDSEKKNASDLLYFSKMIYMLTYFLDGKEINDLLTTLISKFDNIK |
| | EFLKIMKSSAVDVECELTAGYKLFNDSQRITNELFIVKNIASMRKPAASAKLTMFRDALTILGIDDKIT |
| | DDRISEILKLKEKGKGIHGLRNFITNNVIESSRFVYLIKYANAQKIREVAKNEKWMFVLGGIPDTQIE |
| | RYYKSCVEFPDMNSSLEAKCSELARMIKNISFDDFKNVKQQAKGRENVAKERAKAVIGLYLTVMYL |
| | LVKNLVNVNARYVIAIHCLERDFGLYKEIIPELASKNLKNDYRILSQTLCELCDDRDESPNLFLKKNK |
| | RLRKCVEVDINNADSSMTRKYRNCIAHLTVVRELKEYIGDIRTVDSYFSIYHYVMQRCITKREDDTK |
| | QEEKIKYEDDLLKNHGYTKDFVKALNSPFGYNIPRFKNLSIEQLFDRNEYLTEK |
| IMG_ | VEINTSNPTHRSGESSSVRGDMLGLKSELEKRFFGKTFDDNIHIQLIYNILDIEKILAVYVTNIVYALNN |
| 3300014787 | MLGVKGSESYDDFMGYLSAQNTYYIFTHPDKSNLSDKVKGNIKKSLSKFNDLLKTKRLGYFGLEEP |
| SEQ ID NO: | KTKDKRVSEAYKKRVYHMLAIVGQIRQSVFHDKSNELDEYLYSFIDIIDSEYRDTLDYLVDERFDSIN |
| 5047 | KGFVQGNKVNISLLIDMMKGYEADDIIRLYYDFIVLKSQKNLGFSIKKLREKMLDEYGFRFKDKQYD |
| | SVRSKMYKLMDFLLFCNYYRNDVVAGEALVRKLRFSMTDDEKEGIYADEAEKLWGKFRNDFENIA |
| | DHMNGDVTKELGKADMDFDEKILDSEKKNASDLLYFSKMIYMLTYFLDGKEINDLLTTLISKFDNIK |
| | EFLKIMKSSAVDVECELTAGYKLFNDSQRITNELFIVKNIASMRKPAASAKLTMFRDALTILGIDDKIT |
| | DDRISEILKLKEKGKGIHGLRNFITNNVIESSRFVYLIKYANAQKIREVAKNEKVVMFVLGGIPDTQIE |
| | RYYKSCVEFPDMNSSLEAKCSELARMIKNISFD |
| OJNJ01.1 | VEINTSNPTHRSGESSPVRWDMLGLKSELEKRFFGKTFDDNIHIQLIYNILDIEKILAVYVTNIVYALN |
| SEQ ID NO: | NMLGIKDSESYDDFMGYLSARNTYEVFTHPDKSNLSDKVKGNIKKSLSKFNVLLKTKRLGYFGLEEP |
| 5048 | KTKDTNALEAYKKRVYHMLAIVGQIRQSVFHDKSSKLDEDLYSFIDIIDSEYRETLDYLVDERFDSIN |
| | KGFIQGNKVNISLLIDMMKDDYEADDIIRLYYDFIVLKSQKNLGFSIKKLREKMLDEYGFRFKDKQY |
| | DSVRSKMYKLMDFLLFCNYYRNDVVAGEALVRKLRFSMTDDEKEGIYADEAEKLWGKFRNDFENI |
| | ADHMNGDVTKELGKADMDFDEKILDSEKKNASDLLYFSKMIYMLTYFLDGKEINDLLTTLISKFDNI |
| | KEFLKIMKSSAVDVECELTAGYKLFNDSQRITNELFIVKNIASMRKPAASAKLTMFRDALTILGIDDNI |
| | TDDRISEILKLKEKGKGIHGLRNFITNNVIESSRFVYLIKYANAQKIREVAENEKVVMFVLGGIPDTQI |
| | ERYYKSCVEFPDMNSSLEAKRSELARMIKNISFDDFKNVKQQAKGRENVAKERAKAVIGLYLTVMY |
| | LLVKNLVNVNARYVIAIHCLERDFGLYKEIIPELASKNLKNDYRILSQTLCELCDDRDESPNLFLKKN |
| | KRLRKCVEVDINNADSSMTRKYRNCIAHLTVVRELKEYIGDIRTVDSYFSIYHYVMQRCITKRGDDT |
| | KQEEKIKYEDDLLKNHGYTKDFVKALNSPFGYNIPRFKNLSIEQLFDRNE |
| UXQD01.1 | MPAAEAAAPAAEKKKSSVKAAGMKSILVSKNKMYITSFGKGNSAVLEYEVDKVDNDNYNKTQLSS |
| SEQ ID NO: | KDNSNIELGDVDEVNITFSSKHGFESGVEINTSNPTHRSGESSPVRWDMLGLKSELEKRFFGKTFDDN |
| 5049 | IHIQLIYNILDIEKILAVYVTNIVYALNNMLGIKKSESYDDFMGYLSARNTYEVFTHPDKSNLSDKVK |
| | GNIKKSLSKFNVLLKTKRLGYFGLEEPKTKDTNALEAYKKRVYHMLAIVGQIRQSVFHDKSSKLHED |
| | LYSFIDIIDSEYRETLDYLVDERFDSINKGFIQGNKVNISLLIDMMKDDYEADDIIRLYYDFIVLKSQKN |
| | LGFSIKKLREKMLDEYGFRFKDKQYDSVRSKMYKLMDFLLFCNYYRNDVVAGEALVRKLRFSMTD |
| | DEKEGIYADEAEKLWGKFRNDFENIADHMNGDVIKELGKADMDFDEKILDSEKKNASDLLYFSKMI |
| | YMLTYFLDGKEINDLLTTLISKFDNIKEFLKIMKSSAVNVECELTAGYKLFNDSQRITNELFIVKNIAS |
| | MRKPAASAKLTMFRDALTILGIDDKITDDRISEILKLKEKGKGIHGLRNFITNNVIESSRFVYLIKYAN |
| | AQKIREVAKNEKVVMFVLGGIPDTQIERYYKSCVEFPDMNSSLEAKRSELARMIKNISFDDFKNVKQ |
| | QAKGRENVAKERAKAVIGLYLTVMYLLVKNLVNVNARYVIAIHCLERDFGLYKEIIPELASKNLKN |
| | DYRILSQTLCELCDDRDESPNLFLKKNRRLRKCVEVDINNADSSMTRKYRNCIAHLTVVRELKEYIG |
| | DIRTVDSYFSIYHYVMQRCITKREDDTKQEDKIKYEDDLLKNHGYTKDFVKALNSPFGYNIPRFKNL |
| | SIEQLFDRNEYLTEK |
| OPGR01.1 | MPAAEAAAPAAEKKKSSVKAAGMKSILVSKNKMYITSFGKGNSAVLEYEVDKVDNDNYNKTQLSS |
| SEQ ID NO: | KDNSNIELGDVDEVNITFSSKHGFESGVEINTSNPTHRSGESSPVRWDMLGLKSELEKRFFGKTFDDN |
| 5050 | IHIQLIYNILDIEKILAVYVTNIVYALNNMLGIKKSESYDDFMGYLSARNTYEVFTHPDKSNLSDKVK |
| | GNIKKSLSKFNVLLKTKRLGYFGLEEPKTKDTNALEAYKKRVYHMLAIVGQIRQSVFHDKSSKLHED |
| | LYSFIDIIDSEYRETLDYLVDERFDSINKGFIQGNKVNISLLIDMMKDDYEADDIIRLYYDFIVLKSQKN |
| | LGFSIKKLREKMLDEYGFRFKDKQYDSVRSKMYKLMDFLLFCNYYRNDVVAGEALVRECLRFSMTD |
| | DEKEGIYADEAEKLWGKFRNDFENIADHMNGDVIKELGKADMDFDEKILDSEKKNASDLLYFSKMI |
| | YMLTYFLDGKEINDLLTTLISKFDNIKEFLKIMKSSAVNVECELTAGYKLFNDSQRITNELFIVKNIAS |
| | MRKPAASAKLTMFRDALTILGIDDKITDDRISEILKLKEKGKGIHGLRNFITNNVIESSRFVYLIKYAN |
| | AQKIREVAKNEKVVMFVLGGIPDTQIERYYKSCVEFPDMNSSLEAKRSELARMIKNISFDDFKNVKQ |
| | QAKGRENVAKERAKAVIGLYLTVMYLLVKNLVNVNARYVIAIHCLERDFGLYKEIIPELASKNLKN |
| | DYRILSQTLCELCDDRDESPNLFLKKNRRLRKCVEVDINNADSSMTRKYRNCIAHLTVVRELKEYIG |
| | DIRTVDSYFSIYHYVMQRCITKREDDTKQEDKIKYEDDLLKNHGYTKDFVKALNSPFGYNIPRFKNL |
| | SIEQLFDRNEYLTEK |
| UXVN01.1 | MPAAEAAAPAAEKKKSSVKAAGMKSILVSKNKMYITSFGKGNSAVLEYEVDKVDNDNYNKTQLSS |
| SEQ ID NO: | KDNSNIELGDVDEVNITFSSKHGFESGVEINTSNPTHRSGESSPVRWDMLGLKSELEKRFFGKTFDDN |
| 5051 | IHIQLIYNILDIEKILAVYVTNIVYALNNMLGIKKSESYDDFMGYLSARNTYEVFTHPDKSNLSDKVK |
| | GNIKKSLSKFNVLLKTKRLGYFGLEEPKTKDTNALEAYKKRVYHMLAIVGQIRQSVFHDKSSKLHED |
| | LYSFIDIIDSEYRETLDYLVDERFDSINKGFIQGNKVNISLLIDMMKDDYEADDIIRLYYDFIVLKSQKN |
| | LGFSIKKLREKMLDEYGFRFKDKQYDSVRSKMYKLMDFLLFCNYYRNDVVAGEALVRKLRFSMTD |
| | DEKEGIYADEAEKLWGKFRNDFENIADHMNGDVIKELGKADMDFDEKILDSEKKNASDLLYFSKMI |
| | YMLTYFLDGKEINDLLTTLISKFDNIKEFLKIMKSSAVNVECELTAGYKLFNDSQRITNELFIVKNIAS |
| | MRKPAASAKLTMFRDALTILGIDDKITDDRISEILKLKEKGKGIHGLRNFITNNVIESSRFVYLIKYAN |
| | AQKIREVAKNEKVVMFVLGGIPDTQIERYYKSCVEFPDMNSSLEAKRSELARMIKNISFDDFKNVKQ |
| | QAKGRENVAKERAKAVIGLYLTVMYLLVKNLVNVNARYVIAIHCLERDFGLYKEIIPELASKNLKN |
| | DYRILSQTLCELCDDRDESPNLFLKKNRRLRKCVEVDINNADSSMTRKYRNCIAHLTVVRELKEYIG |
| | DIRTVDSYFSIYHYVMQRCITKREDDTKQEDKIKYEDDLLKNHGYTKDFVKALNSPFGYNIPRFKNL |
| | SIEQLFDRNEYLTEK |
| ULMK01.1 | MPAAEAAAPAAEKKKSSVKAAGMKSILVSKNKMYITSFGKGNSAVLEYEVDKVDNDNYNKTQLSS |
| SEQ ID NO: | KDNSNIELGDVDEVNITFSSKHGFESGVEINTSNPTHRSGESSPVRWDMLGLKSELEKRFFGKTFDDN |
| 5052 | IHIQLIYNILDIEKILAVYVTNIVYALNNMLGIKKSESYDDFMGYLSARNTYEVFTHPDKSNLSDKVK |
| | GNIKKSLSKFNVLLKTKRLGYFGLEEPKTKDTNALEAYKKRVYHMLAIVGQIRQSVFHDKSSKLHED |
| | LYSFIDIIDSEYRETLDYLVDERFDSINKGFIQGNKVNISLLIDMMKDDYEADDIIRLYYDFIVLKSQKN |
| | LGFSIKKLREKMLDEYGFRFKDKQYDSVRSKMYKLMDFLLFCNYYRNDVVAGEALVRKLRFSMTD |
| | DEKEGIYADEAEKLWGKFRNDFENIADHMNGDVIKELGKADMDFDEKILDSEKKNASDLLYFSKMI |
| | YMLTYFLDGKEINDLLTTLISKFDNIKEFLKIMKSSAVNVECELTAGYKLFNDSQRITNELFIVKNIAS |
| | MRKPAASAKLTMFRDALTILGIDDKITDDRISEILKLKEKGKGIHGLRNFITNNVIESSRFVYLIKYAN |
| | AQKIREVAKNEKVVMFVLGGIPDTQIERYYKSCVEFPDMNSSLEAKRSELARMIKNISFDDFKNVKQ |
| | QAKGRENVAKERAKAVIGLYLTVMYLLVKNLVNVNARYVIAIHCLERDFGLYKEIIPELASKNLKN |
| | DYRILSQTLCELCDDRDESPNLFLKKNRRLRKCVEVDINNADSSMTRKYRNCIAHLTVVRELKEYIG |
| | DIRTVDSYFSIYHYVMQRCITKREDDTKQEDKIKYEDDLLKNHGYTKDFVKALNSPFGYNIPRFKNL |
| | SIEQLFDRNEYLTEK |
| UXSY01.1 | MPAAEAAAPAAEKKKSSVKAAGMKSILVSKNKMYITSFGKGNSAVLEYEVDKVDNDNYNKTQLSS |
| SEQ ID NO: | KDNSNIELGDVDEVNITFSSKHGFESGVEINTSNPTHRSGESSPVRWDMLGLKSELEKRFFGKTFDDN |
| 5053 | IHIQLIYNILDIEKILAVYVTNIVYALNNMLGIKKSESYDDFMGYLSARNTYEVFTHPDKSNLSDKVK |
| | GNIKKSLSKFNVLLKTKRLGYFGLEEPKTKDTNALEAYKKRVYHMLAIVGQIRQSVFHDKSSKLHED |
| | LYSFIDIIDSEYRETLDYLVDERFDSINKGFIQGNKVNISLLIDMMKDDYEADDIIRLYYDFIVLKSQKN |
| | LGFSIKKLREKMLDEYGFRFKDKQYDSVRSKMYKLMDFLLFCNYYRNDVVAGEALVRKLRFSMTD |
| | DEKEGIYADEAEKLWGKFRNDFENIADHMNGDVIKELGKADMDFDEKILDSEKKNASDLLYFSKMI |
| | YMLTYFLDGKEINDLLTTLISKFDNIKEFLKIMKSSAVNVECELTAGYKLFNDSQRITNELFIVKNIAS |
| | MRKPAASAKLTMFRDALTILGIDDKITDDRISEILKLKEKGKGIHGLRNFITNNVIESSRFVYLIKYAN |
| | AQKIREVAKNEKVVMFVLGGIPDTQIERYYKSCVEFPDMNSSLEAKRSELARMIKNISFDDFKNVKQ |
| | QAKGRENVAKERAKAVIGLYLTVMYLLVKNLVNVNARYVIAIHCLERDFGLYKEIIPELASKNLKN |
| | DYRILSQTLCELCDDRDESPNLFLKKNRRLRKCVEVDINNADSSMTRKYRNCIAHLTVVRELKEYIG |
| | DIRTVDSYFSIYHYVMQRCITKREDDTKQEDKIKYEDDLLKNHGYTKDFVKALNSPFGYNIPRFKNL |
| | SIEQLFDRNEYLTEK |
| OPHJ01.1 | MPAAEAAAPAAEKKKSSVKAAGMKSILVSKNKMYITSFGKGNSAVLEYEVDKVDNDNYNKTQLSS |
| SEQ ID NO: | KDNSNIELGDVDEVNITFSSKHGFESGVEINTSNPTHRSGESSPVRWDMLGLKSELEKRFFGKTFDDN |
| 5054 | IHIQLIYNILDIEKILAVYVTNIVYALNNMLGIKKSESYDDFMGYLSARNTYEVFTHPDKSNLSDKVK |
| | GNIKKSLSKFNVLLKTKRLGYFGLEEPKTKDTNALEAYKKRVYHMLAIVGQIRQSVFHDKSSKLHED |
| | LYSFIDIIDSEYRETLDYLVDERFDSINKGFIQGNKVNISLLIDMMKDDYEADDIIRLYYDFIVLKSQKN |
| | LGFSIKKLREKMLDEYGFRFKDKQYDSVRSKMYKLMDFLLFCNYYRNDVVAGEALVRKLRFSMTD |
| | DEKEGIYADEAEKLWGKFRNDFENIADHMNGDVIKELGKADMDFDEKILDSEKKNASDLLYFSKMI |
| | YMLTYFLDGKEINDLLTTLISKFDNIKEFLKIMKSSAVNVECELTAGYKLFNDSQRITNELFIVKNIAS |
| | MRKPAASAKLTMFRDALTILGIDDKITDDRISEILKLKEKGKGIHGLRNFITNNVIESSRFVYLIKYAN |
| | AQKIREVAKNEKVVMFVLGGIPDTQIERYYKSCVEFPDMNSSLEAKRSELARMIKNISFDDFKNVKQ |
| | QAKGRENVAKERAKAVIGLYLTVMYLLVKNLVNVNARYVIAIHCLERDFGLYKEIIPELASKNLKN |
| | DYRILSQTLCELCDDRDESPNLFLKKNRRLRKCVEVDINNADSSMTRKYRNCIAHLTVVRELKEYIG |
| | DIRTVDSYFSIYHYVMQRCITKREDDTKQEDKIKYEDDLLKNHGYTKDFVKALNSPFGYNIPRFKNL |
| | SIEQLFDRNEYLTEK |
| UPPC01.1 | MKSSAVNVECELTAGYKLFNDSQRITNELFIVKNIASMRKPAASAKLTMFRDALTILGIDDNITDDRI |
| SEQ ID NO: | SEILKLKEKGKGIHGLRNFITNNVIESSRFVYLIKYANAQKIREVAKNEKVVMFVLGGIPDTQIERYYK |
| 5055 | SCVEFPDMNSSLEAKRSELARMIKNISFDDFKNVKQQAKGRENVAKERAKAVIGLYLTVMYLLVKN |
| | LVNVNARYVIAIHCLERDFGLYKEIIPELASKNLKNDYRTLSQTLCGLCDKSPNLFLKKNERLRKCVE |
| | VDINNADSSMTRKYRNCIAHLTVVRELKEYIGDIRTVDSYFSIYHYVMQRCITKRENDTKQEDKIKY |
| | EDDLLKNHGYTKDFVKALNSPFGYNIPRFKNLSIEQLFDRNEYLTEK |
| OWCF01.1_2 | MKSSAVNVECELTAGYKLFNDSQRITNELFIVKNIASMRKPAASAKLTMFRDALTILGIDDNITDDRI |
| SEQ ID NO: | SEILKLKEKGKGIHGLRNFITNNVIESSRFVYLIKYANAQKIREVAKNEKVVMFVLGGIPDTQIERYYK |
| 5056 | SCVEFPDMNSSLEAKRSELARMIKNISFDDFKNVKQQAKGRENVAKERAKAVIGLYLTVMYLLVKN |
| | LVNVNARYVIAIHCLERDFGLYKEIIPELASKNLKNDYRTLSQTLCGLCDKSPNLFLKKNERLRKCVE |
| | VDINNADSSMTRKYRNCIAHLTVVRELKEYIGDIRTVDSYFSIYHYVMQRCITKRENDTKQEDKIKY |
| | EDDLLKNHGYTKDFVKALNSPFGYNIPRFKNLSIEQLFDRNEYLTEK |
| OGLN01.1_2 | MKSSAVNVECELTAGYKLFNDSQRITNELFIVKNIASMRKPAASAKLTMFRDALTILGIDDNITDDRI |
| SEQ ID NO: | SEILKLKEKGKGIHGLRNFITNNVIESSRFVYLIKYANAQKIREVAKNEKVVMFVLGGIPDTQIERYYK |
| 5057 | SCVEFPDMNSSLEAKRSELARMIKNISFDDFKNVKQQAKGRENVAKERAKAVIGLYLTVMYLLVKN |
| | LVNVNARYVIAIHCLERDFGLYKEIIPELASKNLKNDYRTLSQTLCGLCDKSPNLFLKKNERLRKCVE |
| | VDINNADSSMTRKYRNCIAHLTVVRELKEYIGDIRTVDSYFSIYHYVMQRCITKRENDTKQEDKIKY |
| | EDDLLKNHGYTKDFVKALNSPFGYNIPRFKNLSIEQLFDRNEYLTEK |
| OGWR01.1 | MKSSAVNVECELTAGYKLFNDSQRITNELFIVKNIASMRKPAASAKLTMFRDALTILGIDDNITDDRI |
| SEQ ID NO: | SEILKLKEKGKGIHGLRNFITNNVIESSRFVYLIKYANAQKIREVAKNEKVVMFVLGGIPDTQIERYYK |
| 5058 | SCVEFPDMNSSLEAKRSELARMIKNISFDDFKNVKQQAKGRENVAKERAKAVIGLYLTVMYLLVKN |
| | LVNVNARYVIAIHCLERDFGLYKEIIPELASKNLKNDYRTLSQTLCGLCDKSPNLFLKKNERLRKCVE |
| | VDINNADSSMTRKYRNCIAHLTVVRELKEYIGDIRTVDSYFSIYHYVMQRCITKRENDTKQEDKIKY |
| | EDDLLKNHGYTKDFVKALNSPFGYNIPRFKNLSIEQLFDRNEYLTEK |
| OHAD01.1_2 | MKSSAVNVECELTAGYKLFNDSQRITNELFIVKNIASMRKPAASAKLTMFRDALTILGIDDNITDDRI |
| SEQ ID NO: | SEILKLKEKGKGIHGLRNFITNNVTESSRFVYLIKYANAQKIREVAKNEKWMFVLGGIPDTQIERYYK |
| 5059 | SCVEFPDMNSSLEAKRSELARMIKNISFDDFKNVKQQAKGRENVAKERAKAVIGLYLTVMYLLVKN |
| | LVNVNARYVIAIHCLERDFGLYKEIIPELASKNLKNDYRTLSQTLCGLCDKSPNLFLKKNERLRKCVE |
| | VDINNADSSMTRKYRNCIAHLTVVRELKEYIGDIRTVDSYFSIYHYVMQRCITKRENDTKQEDKIKY |
| | EDDLLKNHGYTKDFVKALNSPFGYNIPRFKNLSIEQLFDRNEYLTEK |
| USXY01.1 | MKSSAVNVECELTAGYKLFNDSQRITNELFIVKNIASMRKPAASAKLTMFRDALTILGIDDNITDDRI |
| SEQ ID NO: | SEILKLKEKGKGIHGLRNFITNNVIESSRFVYLIKYANAQKIREVAKNEKVVMFVLGGIPDTQIERYYK |
| 5060 | SCVEFPDMNSSLEAKRSELARMIKNISFDDFKNVKQQAKGRENVAKERAKAVIGLYLTVMYLLVKN |
| | LVNVNARYVIAIHCLERDFGLYREIIPELASKNLKNDYRTLSQTLCGLCDKSPNLFLKKNERLRKCVE |
| | VDINNADSSMTRKYRNCIAHLTVVRELKEYIGDIRTVDSYFSIYHYVMQRCITKRENDTKQEDKIKY |
| | EDDLLKNHGYTKDFVKALNSPFGYNIPRFKNLSIEQLFDRNEYLTEK |
| UZLM01.1_2 | MKSSAVNVECELTAGYKLFNDSQRITNELFIVKNIASMRKPAASAKLTMFRDALTILGIDDNITDDRI |
| SEQ ID NO: | SEILKLKEKGKGIHGLRNFITNNVIESSRFVYLIKYANAQKIREVAKNEKVVMFVLWGIPDTQIERYY |
| 5061 | KSCVEFPDMNSSLEAKRSELARMIKNISFDDFKNVKQQAKGRENVAKERAKAVIGLYLTVMYLLVK |
| | NLVNVNARYVIAIHCLERDFGLYKEIIPELASKNLKNDYRTLSQTLCGLCDKSPNLFLKKNERLRKCV |
| | EVDINNADSSMTRKYRNCIAHLTVVRELKEYIGDIRTVDSYFSIYHYVMQRCITKRENDTKQEDKIK |
| | YEDDLLKNHGYTKDFVKALNSPFGYNIPRFKNLSIEQLFDRNEYLTEK |
| OWCZ01.1 | MRLQVQPLCEEPKTKDNRVSEAYKKRVYHMLAIVGQIRQSVFHDKSSKLHEDLYSFIDIIDSEYRETL |
| SEQ ID NO: | DYLVDERFDSINKGFIQGNKVNISLLIDMMKGYEADDIIRLYYDFIVTKSQKNLGFSIKKLREKMLDE |
| 5062 | YGFRFKDKQYDSVRSKMYKLMDFLLFCNYYRNDVVAGEALVRKLRFSMTDDEKEGIYADEAEKL |
| | WGKFRNDFENIADHMNGDVTKELGKADMDFDEKILDSEKKNASDLLYFSKMIYMLTYFLDGKEIND |
| | LLTTLISKFDNIKEFLKIMKSSAVNVECELTAGYKLFNDSQRITNELFIVKNIASMRKPAASAKLTMFR |
| | DALTILGIDDKITDDRISEILKLKEKGKGIHGLRNFITNNVIESSRFVYLIKYANAQKIREVAKNEKVV |
| | MFVLGGIPDTQIERYYKSCVEFPDMNSSLEAKRSELARMIKNISFDDFKNVKQQAKGRENVAKERAK |
| | AVIGLYLTVMYLLVKNLVNVNARYVIAIHCLERDFGLYKEIIPELASKNLKNDYRILSQTLCELCDKS |
| | PNLFLKKNKRLRKCVEVDINNADSSMTRKYRNCIAHLTVVRELKEYIGDIRTVDSYFSIYHYVMQRC |
| | ITKRENDTKQEDKIKYEDDLLKNHGYTKDFVKALNSPFGYNIPRFKNLSIEQLFDRNEYLTEK |
| OJME01.1 | VEINTSNPTHRSGESSPVRWDMLGLKSELEKRFFGKTFDDNIHIQLIYNILDIEKILAVYVTNIVYALN |
| SEQ ID NO: | NMLGIKDSESYDDFMGYLSAKNTYEVFTHPDKSDLSDKVKGNIKKSFSTFNDLLKTKRLGYFGLEEP |
| 5063 | KTKDTRVSQAYKKRVYHMLAIVGQIRQCVFHDKSGAKRFDLYSFINNIDPEYRDTLDYLVDERFDSI |
| | NKGFIQGNKVNISLLIDMMKGYEADDIIRFYYDFIVLKSQKNLGFSIKKLREKMLDEYGFRFKDKQY |
| | DSVRSKMYKLMDFLLFCNYYRNDVAAGEALVRKLRFSMTDDEKEGIYADEAAKLWGKFRNDFENI |
| | AGHMNGDVTKELGKADMDFDEKTLDSEKKNASDLLYFSKMIYMLTYFLDGKEINDLLTTLISKFDNI |
| | KEFLKIMKSSAVDVECELTAGYKLFNDSQRITNELFIVKNIASMRKPAASAKLTMFRDALTILGIDDNI |
| | TDDRISEILKLKEKGKGIHGLRNFITNNVIESSRFVYLIKYANAQKIREVAKNEKVVMFVLGGIPDTQI |
| | ERYYKSCVEFPDMNSSLEAKRSELARMIKNISFDDFKNVKQQAKGRENVAKERAKAVIGLYLTVMY |
| | LLVKNLVNVNARYVIAIHCLERDFGLYKEIIPELASKNLKNDYRILSQTLCELCDKSPNLFLKKNERL |
| | RKCVEVDINNADSIMTRKYRNCIAHLTVVRELKEYIGDIRTVDSYFSIYHYVMQRCITKRENDTKQE |
| | DKIKYEDDLLKNHGYTKDFVKALNSPFGYNIPRFKNLSIEQLFDRNEYLTEK |
| UPFI01.1 | VEINTSNPTHRSGESSPVRWDMLGLKSELEKRFFGKTFDDNIHIQLIYNILDIEKILAVYVTNIVYALN |
| SEQ ID NO: | NMLGIKDSESYDDFMGYLSAKNTYEVFTHPDKSDLSDKVKGNIKKSFSTFNDLLKTKRLGYFGLEEP |
| 5064 | KTKDTRVSQAYKKRVYHMLAIVGQIRQCVFHDKSGAKRFDLYSFINNIDPEYRDTLDYLVDERFDSI |
| | NKGFIQGNKVNISLLIDMMKGYEADDIIRFYYDFIVLKSQKNLGFSIKKLREKMLDEYGFRFKDKQSV |
| | RSKMYKLMDFLLFCNYYRNDVAAGEALVRKLRFSMTDDEKEGIYADEAAKLWGKFRNDFENIAGH |
| | MNGDVTKELGKADMDFDEKTLDSEKKNASDLLYFSKMIYMLTYFLDGKEINDLLTTLISKFDNIKEF |
| | LKIMKSSAVDVECELTAGYKLFNDSQRITNELFIVKNIASMRKPAASAKLTMFRDALTILGIDDNITD |
| | DRISEILKLKEKGKGIHGLRNFITNNVIESSRFVYLIKYANAQKIREVAKNEKWMFVLGGIPDTQIER |
| | YYKSCVEFPDMNSSLEAKRSELARMIKNISFDDFKNVKQQAKGRENVAKERAKAVIGLYLTVMYLL |
| | VKNLVNVNARYVIAIHCLERDFGLYKEIIPELASKNLKNDYRILSQTLCELCDKSPNLFLKKNERLRK |
| | CVEVDINNADSIMTRKYRNCIAHLTVVRELKEYIGDIRTVDSYFSIYHYVMQRCITKRENDTKQEDKI |
| | KYEDDLLKNHGYTKDFVKALNSPFGYNIPRFKNLSIEQLFDRNEYLIEK |
| OJAG01.1 | MTDDEKEGIYADEAAKLWGKFRNDFENIADHMNGDVIKELGKADMDFDEKILDSEKKNASDLLYF |
| SEQ ID NO: | SKMIYMLTYFLDGKEINDLLTTLISKFDNIKEFLKIMKSSAVDVECELTAGYKLFNDSQRITNELFIVK |
| 5065 | NIASMRKPAASAKLTMFRDALTILGIDDKITDDRISEILKLKEKGKGIHGLRNFITNNVIESSRFVYLIK |
| | YANAQKIREVAKNEKVVMFVLGGIPDTQIERYYKSCVEFPDMNSSLGVKRSELARMIKNISFDDFKN |
| | VKQQSKGRENVAKERAKAVIGLYLTVMYLLVKNLVNVNARYVIAIHCLERDFGLYKEIIPELASKNL |
| | KNDYRILSQTLCELCDKSPNLFLKKNERLRKCVEVDINNADSSMTRKYRNCIAHLTVVRELKEYIGDI |
| | CTVDSYFSIYHYVMQRCITKRENDTKQEEKIKYEDDLLKNHGYTKDFVKALNSPFGYNIPRFKNLSIE |
| | QLFDRNEYLTEK |
| UBIF01.1 | MPAAEAAAPAAEKKKSSVKAAGMKSILVSENKMYITSFGKGNSAVLEYEVDNNDYNKTQLSSKDN |
| SEQ ID NO: | SNIELCDVGKVNITFSSRRGFESGVEINTSNPTHRSGESSSVRGDMLGLKSELEKRFFGKNFDDNIHIQ |
| 5066 | LIYNILDIEKILAVYVTNIVYALNNMLGEGDESNYDFMGYLSTFNTYKVFTNPNGSTLSDDKKENIRK |
| | SLSKFNALLKTKRLGYFGLEEPKTKDTRASEAYKKRVYHMLAIVGQIRQCVFHDKSGAKRFDLYSFI |
| | NNIDPEYRETLDYLVDERFDSINKGFIQGNKVNISLLIDMMKGYEADDIIRLYYDFIVLKSQKNLGFSI |
| | KKLREKMLDEYGFRFKDKQYDSVRSKMYKLMDFLLFCNYYRNDIAAGESLVRKLRFSMTDDEKEG |
| | IYADEAAKLWGKFRNDFENIADHMNGDVIKELGKADMDFDEKILDSEKKNASDLLYFSKMIYMLTY |
| | FLDGKEINDLLTTLISKFDNIKEFLKIMKSSAVDVECELTAGYKLFNDSQRITNELFIVKNIASMRKPA |
| | ASAKLTMFRDALTILGIDDKITDDRISEILKLKEKGKGIHGLRNFITNNVIESSRFVYLIKYANAQKIRE |
| | VAKNEKVVMFVLGGIPDTQIERYYKSCVEFPDMNSSLGVKRSELARMIKNISFDDFKNVKQQSKGRE |
| | NVAKERAKAVIGLYLTVMYLLVKNLVNVNARYVIAIHCLERDFGLYKEIIPELASKNLKNDYRILSQ |
| | TLCELCDKSPNLFLKKNERLRKCVEVDINNADSSMTRKYRNCIAHLTVVRELKEYIGDICTVDSYFSI |
| | YHYVMQRCITKRENDTKQEEKIKYEDDLLKNHGYTKDFVKALNSPFGYNIPRFKNLSIEQLFDRNEY |
| | LTEK |
| CEAG01.1_2 | MRKPAASAKLTMFRDALTILGIDDNITDDRISEILKLKEKGKGIHGLRNFITNNVIESSRFVYLIKYAN |
| SEQ ID NO: | AQKIREVAKNEKVVMFVLGGIPDTQIERYYKSCVEFPDMNSSLGVKRSELARMIKNISFDDFKNVKQ |
| 5067 | QAKGRENVAKERAKAVIGLYLTVMYLLVKNLVNVNARYVIAIHCLERDFGLYKEIIPELASKNLKN |
| | DYRILSQTLCELCDKSPNLFLKKNERLRKCVEVDINNADSSMTRKYRNCIAHLTVVRELKEYIGDICT |
| | VDSYFSIYHYVMQRCITKRENDTKQEEKIKYEDDLLKNHGYTKDFVKALNSPFGYNIPRFKNLSIEQL |
| | FDRNEYLTEK |
| CEAH01.1_2 | MRKPAASAKLTMFRDALTILGIDDNITDDRISEILKLKEKGKGIHGLRNFITNNVIESSRFVYLIKYAN |
| SEQ ID NO: | AQKIREVAKNEKVVMFVLGGIPDTQIERYYKSCVEFPDMNSSLGVKRSELARMIKNISFDDFKNVKQ |
| 5068 | QAKGRENVAKERAKAVIGLYLTVMYLLVKNLVNVNARYVIAIHCLERDFGLYKEIIPELASKNLKN |
| | DYRILSQTLCELCDKSPNLFLKKNERLRKCVEVDINNADSSMTRKYRNCIAHLTVVRELKEYIGDICT |
| | VDSYFSIYHYVMQRCITKRENDTKQEEKIKYEDDLLKNHGYTKDFVKALNSPFGYNIPRFKNLSIEQL |
| | FDRNEYLTEK |
| CEAF01.1_2 | MRKPAASAKLTMFRDALTILGIDDNITDDRISEILKLKEKGKGIHGLRNFITNNVIESSRFVYLIKYAN |
| SEQ ID NO: | AQKIREVAKNEKVVMFVLGGIPDTQIERYYKSCVEFPDMNSSLGVKRSELARMIKNISFDDFKNVKQ |
| 5069 | QAKGRENVAKERAKAVIGLYLTVMYLLVKNLVNVNARYVIAIHCLERDFGLYKEIIPELASKNLKN |
| | DYRILSQTLCELCDKSPNLFLKKNERLRKCVEVDINNADSSMTRKYRNCIAHLTVVRELKEYIGDICT |
| | VDSYFSIYHYVMQRCITKRENDTKQEEKIKYEDDLLKNHGYTKDFVKALNSPFGYNIPRFKNLSIEQL |
| | FDRNEYLTEK |
| GCA_ | MPAVEVIAPAAEKKKSSVKAAGMKSILVSENKMYITSFGKGNSAVLEYEVDNNDYNKTQLSSKDNS |
| 003460765.1_ | NIELGNVNEVNITFSSRRGFESGVEINTSNPTHRSGESSSVRGDMLGLKSELEKRFFGKTFDDNIHIQLI |
| ASM346076v1_ | YNILDIEKILAVYVTNIVYALNNMLGEGGDESHDDFMGYLSAKNTYDVFTNPNGSTLSDDKKENIRK |
| genomic | SLRKFNDLLKTKRLGYFGLEEPKTKDTRVSQAYKKRVYHMLAIVGQIRQCVFHDLSEHSEYDLYSFI |
| SEQ ID NO: | DNSKKVYRECRETLDYLVDERFDSINKGFIQGNKVNISLLIDMMKGYEADDIIRLYYDFIVLKSQKNL |
| 5070 | GFSIKKLREKMLDEYGFRFKDKQYDSVRSKMYKLMDFLLFCNYYRNDVVAGEALVRKLRFSMTDD |
| | EKEGIYADEAAKLWGKFRNDFENIADHMNGDVIKELGKADMDFDEKILDSEKKNASDLLYFSKMIY |
| | MLTYFLDGKEINDLLTTLISKFDNIKEFLKIMKSSAVDVECELTAGYKLFNDSQRITNELFIVKNIASM |
| | RKPAASARLTMFRDALTILGIDDKITDDRISEILKLKEKGKGIHGLRNFITNNVIESSRFVYLIKYANAQ |
| | KIREVAKNEKVVMFVLGGIPDTQIERYYKSCVEFPDMNSSLEAKRSELARMIKNISFDDFKNVKQQA |
| | KGRENVAKERAKAVIGLYLTVMYLLVKNLVNVNARYVIAIHCLERDFGLYKEIIPELASKNLKNDY |
| | RILSQTLCELCDKSPNLFLKKNERLRKCVEVDINNADSSMTRKYRNCIAHLTVVRELKEYIGDIRTVD |
| | SYFSIYHYVMQRCITKRENDTKQEDKIKYEDDLLKNHGYTKDFVKALNSPFGYNIPRFKNLSIEQLFD |
| | RNEYLTEK |
| OQMA01.1 | MLGVKGSESYDDFMGYLSAQNTYYIFTHPDKSNLSDKVKGNIKKSLSKFNDLLKTKRLGYFGLEEP |
| SEQ ID NO: | KTKDKRVSEAYKKRVYHMLAIVGQIRQSVFHDKSNELDEYLYSFIDIIDSEYRDTLDYLVDERFDSIN |
| 5071 | KGFVQGNKVNISLLIDMMKGYEADDIIRLYYDFIVLKSQKNLGFSIKKLREKMLDEYGFRFKDKQYD |
| | SVRSKMYKLMDFLLFCNYYRNDVVAGEALVRKLRFSMTDDEKEGIYADEAAKLWGKFRNDFENIA |
| | DHMNGDVTKELGKADMDFDEKILDSEKKNASDLLYFSKMIYMLTYFLDGKEINDLLTTLISKFDNIK |
| | EFLKIMESSAVDVECELTAGYKLFNDSQRITNELFIVKNIASMRKPAASAKLTMFRDALTILGIDDKIT |
| | DDRISEILKLKEKGKGIHGLRNFITNNVIESSRFVYLIKYANAQKIREVAKNEKWMFVLGGIPDTQIE |
| | RYYKSCVEFPDMNSSLEAKRSELARMIKNISFDDFKNVKQQAKGRENVAKERAKAVIGLYLTVMYL |
| | LVKNLVNVNARYVIAIHCLERDFGLYKEIIPELASKNLKNDYRILSQTLCELCDKSPNLFLKKNERLR |
| | KCVEVDINNADSSMTRKYRNCIAHLTVVRELKEYIGDIRTVDSYFSIYHYVMQRCITKRENDTKQED |
| | KIKYEDDLLKNHGYTKDFVKALNSPFGYNIPRFKNLSIEQLFDRNEYLTEK |
| OLQX01.1 | MTCQNTQSMTFTALLTIAKKCTESAENLVDERFDSINKGFIQGNKVNISLLIDMMKGYEADDIIRLYY |
| SEQ ID NO: | DFIVLKSQKNLGFSIKKLREKMLDEYGFRFKDKQYDSVRSKMYKLMDFLLFCNYYRNDVVAGEAL |
| 5072 | VRKLRFSMTDDEKEGIYADEAAKLWVKFRNDFENIADHMNGDVIKELGKADMDFDEKILDSEKKN |
| | ASDLLYFSKMIYMLTYFLDGKEINDLLTTLISKFDNIKEFLKIMKSSAVDVECELTAGYKLFNDSQRIT |
| | NELFIVKNIASMRKPAASAKLTMFRDALTILGIDDNITDDRISEILKLKEKGKGIHGLRNFITNNVIESS |
| | RFVYLIKYANAQKIREVAENEKVVMFVLGGIPDTQIERYYKSCVEFPDMNSSLEVKRSELARMIKNIS |
| | FDDFKNVKQQAKGRENVAKERAKAVIGLYLTVMYLLVKNLVNVNARYVIAIHCLERDFGLYKEIIP |
| | ELASKNLKNDYRILSQTLCELCDKSPNLFLKKNERLRKCVEVDINNADSSMTRKYRNCIAHLTVVRE |
| | LKEYIGDIRTVDSYFSIYHYVMQRCITKREDDTKQEEKIKYEDDLLKNHGYTKDFVKALNSPFGYNIP |
| | RFKNLSIEQLFDRNEYLTEK |
| mgm4491403.3_ | LRNFXXXXXXXXXXXXXXXXXXXXXXXXXXXXXXXXXIESSRFVYLIKYANAQKIREVAKNEKV |
| 2 | VMFVLGGIPDTQIERYYKSCVEFPDMNSSLEAKCSELARMIKNISFDDFKNVKQQAKGRENVAKERA |
| SEQ ID NO: | KAVIGLYLTVMYLLVKNLVNVNARYVIAIHCLERDFGLYKEIIPELASKNLKNDYRILSQTLCELCDD |
| 5073 | RDESPNLFLKKNKRLRKCVEVDINNADSSMTRKYRNCIAHLTVVRELKEYIGDIRTVDSYFSIYHYV |
| | MQRCITKREDDTKQEEKIKYEDDLLKNHGYTKDFVKALNSPFGYNIPRFKNLSIEQLFDRNEYLTEK |
| GCA_ | VEINTSNPTHRSGESSPVRWDMLGLKSELEKRFFGKTFDDNIHIQLIYNILDIEKILAVYVTNIVYALN |
| 003460465.1_ | NMLGVKGSESHDDFIGYLSAKNTYEVFTHPDKSNLSDKVKGNIKKSFSTFNDLLKTKRLGYFGLEEP |
| ASM346046v1_ | KTKDNRVSEAYKKRVYHMLAIVGQIRQCVFHDLSEHLEYDLYSFIDNSKKVYRECRETLDYLVDER |
| genomic | FDSINKGFIQGNKVNISLLIDMMKGYEADDIIRLYYDFIVLKSQKNLGFSIKKLREKMLDEYGFRFKD |
| SEQ ID NO: | KQYDSVRSKMYKLMDFLLFCNYYRNDVIAGEALVRKLRFSMTDDEKEGIYADEAAKLWGKFKNDF |
| 5074 | ENIADHMNGDVIKELGKADMDFDEKILDSEKKNASDLLYFSKMIYMLTYFLDGKEINDLLTTLISKF |
| | DNIKEFLKIMKSSAVDVECELTAGYKLFNDSQRITNELFIVKNIASMRKPAASAKLTMFRDALTILGID |
| | DNITDDRISEILKLKEKGKGIHGLRNFITNNVIESSRFVYLIKYANAQKIREVAKNEKVVMFVLGGIPD |
| | TQIERYYKSCVEFPDMNSSLEAKRSELARMIKNIRFDDFKNVKQQAKGRENVAKERAKAVIGLYLT |
| | VMYLLVKNLVNVNARYVIAIHCLERDFGLYKEIIPELASKNLKNDYRILSQTLCELCDYRDKSPNLFL |
| | KKNKRLRKCVEVDINNADSSMTRKYRNCIAHLTVVRELKEYIGDIRTVDSYFSIYHYVMQRCITKRE |
| | DDTKQEDKIKYEDNLLKNHGYTKDFVKALNSPFGYNIPRFKNLSIEQLFDRNEYLTEK |
| OVYG01.1 | VEINTSNPTHRSGESSPVRGDMLGLKSELEKRFFGKTFDDNIHIQLIYNILDIEKILAVYVTNIVYALNN |
| SEQ ID NO: | MLGEGDESNYDFMGYLSTFNTYKVFTNPNGSTLSDDKKENIRKSLSKFNALLKTKRLGYFGLEEPKT |
| 5075 | KDKRVSEAYKKRVYHMLAIVGQIRQSVFHDKSNELDEYLYSFIDIIDSEYRDTLDYLVDERFDSINKG |
| | FVQGNKVNISLLIDMMKGYEADDIIRLYYDFIVLKSQKNLGFSIKKLREKMLDEYGFRFKDKQYDSV |
| | RSKMYKLMDFLLFCNYYRNDVAAGEALVRKLRFSMTDGEKEGIYADEAEKLWGKFRNDFENIADH |
| | MNGDVTKELGKADMDFDEKILDSEKKNASDLLYFSKMIYMLTYFLDGKEINDLLTTLISKFDNIKEFL |
| | KIMKSSAVDVECELTAGYKLFNDSQRITNELFIVKNIASMRKPAASAKLTMFRDALTILGIDDNITDD |
| | RISEILKLKEKGKGIHGLRNFITNNVTESSRFVYLIKYANAQKIREVAKNEKWMFVLGGIPDTQIERY |
| | YKSCVEFPDMNSSLEVKRSELARMIKNISFDDFKNVKQQAKGRENVAKERAKAVIGLYLTVMYLLV |
| | KNLVNVNARYVIAIHCLERDFGLYKEIIPELASKNLKNDYRILSQTLCELCDKSPNLFLKKNRRLRKC |
| | VEVDINNADSSMTRKYRNCIAHLTVVRELKEYIGDIRTVDSYFSIYHYVMQRCITKRGDDTKQEEKI |
| | KYEDDLLKNHGYTKDFVKALNSPFGYNIPRFKNLSIEQLFDRNEYLTEK |
| UPUD01.1 | VEINTSNPTHRSGESSPVRGDMLGLKSELEKRFFGKTFDDNIHIQLIYNILDIEKILAVYVTNIVYALNN |
| SEQ ID NO: | MLGEGDESNYDFMGYLSTFNTYKVFTNPNGSTLSDDKKENIRKSLSKFNALLKTKRLGYFGLEEPKT |
| 5076 | KDKRVSEAYKKRVYHMLAIVGQIRQSVFHDKSNELDEYLYSFIDIIDSEYRDTLDYLVDERFDSINKG |
| | FVQGNKVNISLLIDMMKGYEADDIIRLYYDFIVLKSQKNLGFSIKKLREKMLDEYGFRFKDKQYDSV |
| | RSKMYKLMDFLLFCNYYRNDVAAGEALVRKLRFSMTDGEKEGIYADEAEKLWGKFRNDFENIADH |
| | MNGDVTKELGKADMDFDEKILDSEKKNASDLLYFSKMIYMLTYFLDGKEINDLLTTLISKFDNIKEFL |
| | KIMKSSAVDVECELTAGYKLFNDSQRITNELFIVKNIASMRKPAASAKLTMFRDALTILGIDDNITDD |
| | RISEILKLKEKGKGIHGLRNFITNNVTESSRFVYLIKYANAQKIREVAKNEKWMFVLGGIPDTQIERY |
| | YKSCVEFPDMNSSLEVKRSELARMIKNISFDDFKNVKQQAKGRENVAKERAKAVIGLYLTVMYLLV |
| | KNLVNVNARYVIAIHCLERDFGLYKEIIPELASKNLKNDYRILSQTLCELCDKSPNLFLKKNRRLRKC |
| | VEVDINNADSSMTRKYRNCIAHLTVVRELKEYIGDIRTVDSYFSIYHYVMQRCITKRGDDTKQEEKI |
| | KYEDDLLKNHGYTKDFVKALNSPFGYNIPRFKNLSIEQLFDRNEYLTEK |
| IMG_ | MISYAFITISXXXXIVLKSQKNLGFSIKKLREKMLDEYGFRFKDKQYDSVRSKMYKLMDFLLFCNYY |
| 3300014744_2 | RNDVVAGEALVRKLRFSMTDDEKEGIYADEAAKLWGKFRNDFENIADHMNGDVIKELGKADMDF |
| SEQ ID NO: | DEKILDSEKKNASDLLYFSKMIYMLTYFLDGKEINDLLTTLISKFDNIKEFLKIMKSSAVDVECELTAG |
| 5077 | YKLFNDSQRITNELFIVKNIASMRKPAASAKLTMFRDALTILGIDDNITDDRISEILKLKEKGKGIHGL |
| | RNFITNNVIESSRFVYLIKYANAQKIREVAKNEKVVMFVLGGIPDTQIERYYKSCVEFPDMNSSLEVK |
| | RSELARMIKNICFDDFKNVKQQAKGRENVAKERAKAVIGLYLTVMYLLVKNLVNVNARYVIAIHCL |
| | ERDFGLYKEIVSELASKNLKNDYRILSQTLCELCDKSPNLFLKKNERLRKCVEVDINNADSSMTRKY |
| | RNCIAHLTVVRELKEYIGDIRAVDSYFSIYHYVMQRCITKRGNDTKQEDKIKYEDDLLKNHGYTKDF |
| | VKALNSPFGYNIPRFKNLSIEQLFDRNEYLTEK |
| OBII01.1 | MKSILVSKNKMYITSFGKGNSAVLEYEVDNNDYNKTQLSSKDNSNIELRGVTKVNITFSSKHGLESG |
| SEQ ID NO: | VEINTSNPTHRSGESSPVRWDMLGLKSELEKRFFGKTFDDNIHIQLIYNILDIEKILAVYVTNIVYALN |
| 5078 | NMLGVKGSESHDDFIGYLSTNNTYDVFIDPDNSSLSDDKKANVRKSLSKFNALLKTKRLGYFGLEEP |
| | KTKDNRVSEAYKKRVYHMLAIVGQIRQCVFHDKSGAKRFDLYSFINNIDPEYRETLDYLVDERFDSI |
| | NKDFIQGNKVNISLLIDMMKGYEADDIIRLYYDFIVLKSQKNLGFSIKKLREKMLDEYGFRFKDKQY |
| | DSVRSKMYKLMDFLLFCNYYRNDVVAGEALVRKLRFSMTDDEKEGIYADEAAKLWGKFRNDFENI |
| | ADHMNGDVTKELGKADMDFDEKILDSEKKNASDILYFSKMIYMLTYFLDGKEINDLLTTLISKFDNIK |
| | EFLKIMKSSAVDVECELTAGYKLFNDSQRITNELFIVKNIASMRKPAASAKLTMFRDALTILGIDDNIT |
| | DDRISEILKLKEKGKGIHGLRNFITNNVIESSRFVYLIKYANAQKIREVAKNEKWMFVLGGIPDTQIE |
| | RYYKSCVEFPDMNSSLEVKRSELARMIKNISFDDFKNVKQQAKGRENVAKERAKAVIGLYLTVMYL |
| | LVKNLVNVNARYVIAIHCLERDFGLYKEIIPELASKNLKNDYRILSQTLCDDRDESPNLFLKKNKRLR |
| | KCVEVDINNADSSMTRKYRNCIAHLTVVRELKEYIGDIRTVDTYFSIYHYVMQRCITKREDDTKQEE |
| | KIKYEDDLLKNHGYTKDFVKALNSPFGYNIPRFKNLSIEQLFDRNEYLTEK |
| OWFW01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5079 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLDEYGFRFKDKQYD |
| | SVRSKMYKLMDFLLFCNYYRNDVVAGEALVRKLRFSMTDDEKEGIYADEAAKLWGKFRNDFENIA |
| | DHMNGEAIKELGKADMDFDEKILDSEKKNASDLLYFSKMIYMLTYFLDGKEINDLLTTLISKFDNIK |
| | EFLKIMKSSAVDVECELTAGYKLFNDSQRITNELFIVKNIASMRKPAASAKLTMFRDALTILGIDDNIT |
| | DDRISEILKLKEKGKGIHGLRNFITNNVIESSRFVYLIKYANAQKIREVAKNEKVVMFVLGGIPDTQIE |
| | RYYKSCVEFPDMNSSLEAKRSELARMIKNIGFDDFKNVKQQAKGRENVAKERAKAVIGLYLTVMY |
| | LLVKNLVNVNARYVIAIHCLERDFGLYKEIIPELASKNLKNDYRILSQTLCELCDDRDESPNLFLKKN |
| | KRLRKCVEVDINNADSSMTRKYRNCIAHLTVVRELKEYIGENASALKKADTWSSDVYSATNETGFCI |
| | QPAGLLLERENKTALVNANSSTAYWLYDGTQKQLDKVVMFANSNNDIALKNAVKPEGKDYYNAF |
| | SIRCIKE |
| UXUP01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5080 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGLYADEAAKLWGKFRNDFENIAD |
| | HMNGDVTKELGKADMDFDEKILDSEKKNASDLLYFSKMIYMLTYFLDGKEINDLLTTLISKFDNIKEF |
| | LKIMKSSAVNVECELTAGYKLFNDSQRITNELFIVKNIASMRKPAASAKLTMFRDALTILGIDDKITD |
| | DRISEILKLKEKGKGIHGLRNFITNNVIESSRFVYLIKYANAQKIREVAENEKVVMFVLGGIPDTQIER |
| | YYKSCVEFPDMNSSLEAKRSELARMIKNIRFDDFKNVKQQAKGRENVAKERAKAVIGLYLTVMYLL |
| | VKNLVNVNARYVIAIHCLERDFGLYKEIIPELASKNLKNDYRILSQTLCELCDDRDESPNLFLKKNTC |
| | VPVRATDSESAGIS |
| OZEB01.1 | VEINTSNPTHRSGESSPVRGDMLGLKSELEKRFFGKTFDDNIHIQLIYNILDIEKILAVYVTNIVYALNN |
| SEQ ID NO: | MLGVKGSESHDDFIGYLSTNNTYDVFIDPDNSSLSDDKKANVRKSLSKFNVLLKTKRLGYFGLEEPK |
| 5081 | TKDNRVSEAYKKRVYHMLAIVGQIRQCVFHDKSGAKRFDLYSFINNIDPEYRDTLDYLVEERLKSIN |
| | KDFIQGNKVNISLLIDMMKGYEADDIIRLYYDFIVLKSQKNLGFSIKKLREKMLEEYGFRFKDKQYDS |
| | VRSKMYKLMDFLLFCNYYRNDIAAGEALVRKLRFSMTDDEKEGYMLMKRQSFGANSGMILKISPTT |
| UPPS01.1 | VLSGIFVNAFSSKHGFESGVEINTSNPTHRSGESSPVRGDMLGLKSELEKRFFGKTFDDNIHIQLIYNIL |
| SEQ ID NO: | DIEKILAVYVTNIVYALNNMLGVKGSESHDDFIGYLSTNNTYDVFIDPDNSSLSDDKKANVRKSLSKF |
| 5082 | NVLLKTKRLGYFGLEEPKTKDNRVSEAYKKRVYHMLAIVGQIRQCVFHDKSGAKRFDLYSFINNIDP |
| | EYRDTLDYLVEERLKSINKDFIQGNKVNISLLIDMMKGYEADDIIRLYYDFIVLKSQKNLGFSIKKLRE |
| | KMLEEYGFRFKDKQYDSVRSKMYKLMDFLLFCNYYRNDIAAGEALVRKLRFSMTDDEKEGIYADE |
| | AEKLWVKFRNDFENIADHMNGDVIKELGKADMDFDEKILDSEKKNASDLLYFSKMIYMLTYFLDG |
| | KEINDILTTLISKFDNIKEFLKIMKSSAVDVECELTAGYKLFNDSQRITNELFIVKNIASMRKPAASAKL |
| | TMFRDALTILGIDDNITDDRISEILKLKEKGKGIHGRYTFWQSAVFFKGA |
| OQLI01.1 | VLSGIFVNAFSSKHGFESGVEINTSNPTHRSGESSPVRGDMLGLKSELEKRFFGKTFDDNIHIQLIYNIL |
| SEQ ID NO: | DIEKILAVYVTNIVYALNNMLGEGDESNYDFMGYLSTFNTYKVFTNPNGSTLSDDKKENIRKSLSKF |
| 5083 | NALLKTKRLGYFGLEEPKTKDNRVSEAYKKRVYHMLAIVGQIRQCVFHDKSGAKRFDLYSFINNIDP |
| | EYRETLDYLVDERFDSINKGFIQGNKVNISLLSDMMKDDYEADDIIRLYYDFIVLKSQKNLGFSIKKL |
| | REKMLEEYGFRFKDKQYDSVRSKMYKLMDFLLFCNYYRNDVVAGEALVRKLRFSMTDDEKEGIYA |
| | DEAAKLWGKFRNDFENIADHMNGDVIKELGQADMDFDEKILDSEKKNASDLLYFSKMIYMLTYFL |
| | DDKEINDLLTTLISKFDNIKEFLKIMKSSAVDVECELTAGYKLFNDGQRITNELFIVKNIASMRKPAAS |
| | AKLTMFRDALTILGIDDKITDDRISEILKLKEKGKGIHGLRNFITNNVIESSRFVYLIKYANAQKIREVA |
| | ENEKVVMFVLGGIPDTQIERYYKSCVEFPDMNSSVEAKRSELARMIKNIRFDDFKNVKQQAKGREN |
| | VAKERAKAVIGLYLTVMYLLVKNLVNVNARYVIAIHCLERDFGLYKEIIPELASKNLKNDYRILSQT |
| | LCELCDDRDESPNLFLKKNKRLRKCVEVDINNADSNMTRKYRNCIAHLTVVRELKEYIGDIRTVDSY |
| | FSIYHYVMQRCITKRENDTKQEEKVKSEDDLLKNHGYTIDYATAIKSPFGYKI |
| mgm4491403.3 | MLAIVGQIRQSVFHDKSNELDEYLYSFIDIIDSEYRDTLDYLVDERFDSINKGFVQGNKVNISLLIDMM |
| SEQ ID NO: | KGYEADDIIRLYYDFIVLKSQKNLGFSIKKLREKMLDEYGFRFKDKQYDSVRSKMYKLMDFLLFCN |
| 5084 | YYRNDVVAGEALXXXXXXXXXXXXXXXXXXXXXXXXXXXXXXXXXXXXXMTDDEKEGIYADE |
| | AEKLWGKFRNDFENIADHMNGDVIKELGKADMDFDEKILDSEKKNASDLLYFSKMXXXXXXXXX |
| | XXXXXXXXXXXXXXXXXXXXXXXXXXXXXXXXXXTIFLQRLSASLITSRSF |
| USWZ01.1 | MNGDVTKELGKADMDFDEKILDSEKKNASDLLYFSKMIYMLTYFLDGKEINDLLTTLISKFDNIKEFL |
| SEQ ID NO: | KIMKSSAVDVECELTAGYKLFNDSQRITNELFIVKNIASMRKPAASAKLTMFRDALTILGIDDKITDD |
| 5085 | RISEILKLKEKGKGIHGLRNFITNNVTESSRFVYLIKYANAQKIREVAENEKWMFVLGGILDTQIERY |
| | YKSCVEVPDMNSSLEAKRSELARMIKNISFDDFKNVKQQGPRRVGRTAHRRDGRNARRKTLRGFDA |
| | PHPRRRDGQRNLDPDSDTELHDVSVHGRRRRQEFRHRQIRPDDLRGHRQGRNESLLLRHRRPSGLDF |
| | DGRKIL |
| IMG_ | MGKGNHKSIAKASGLKSTFINGNEVTMTSFGKGNSAVLEKKIIDSEVEDLNPDKAFTVEENNVQKGK |
| 3300028833 | LRIKSNRMSDPATADNPVHVSPEKVGKSIKGQDIIGCKDVLEQRYFGQTFDDNIHIQLIYNILDIEKILA |
| SEQ ID NO: | VHVTNATFAVNNIMRIEDTENEDFIGNLSSCNTYDSFRNYESDTNLSPNVKANLKRSNEFFDIIKKDT |
| 5086 | RLGYFGPAFYEKKGKNFVRKPDKSIYHVLALIGNLRQFVVHDTTIITGKEKSRSWLYNIDKQIGPEFIQ |
| | TMTELYNAAVQNIDRDFIETNKVDIRIIHDAFYLIYGSSDWQKIAEEYYRFSIEKSYKNIGFSVKKLRE |
| | EIISTYAVKFENHKYDSVRHKLNKIIDFLIFTSYSTDDISQKVSVLRTCMNDEEKEERFYKPEAKNIWD |
| | KFRDIFNEFIPERINGKAVSELKKEHFSHREINIDSLKISRKNPDSFSKLIYLLTLFLDGKEINDLLTTLIN |
| | KFDNISSFISIMKEMNISCDFTDEYRFFNKSKYICSELRLINSFARMTSPVSLAKREMYREAVEILGTAG |
| | MNEDEKESLLDKVLCIDGNGKYISSKTDRNRDVNLRNFIANNVIESSRFKYLIRYNNAKKTRVLASN |
| | KTVVRFLLERIDELNEKQIDRYYETCSSDKTLKNKKDKIDFLTELIIKIDCSQFLKVKNRVRAGTAEA |
| | QEKERLKAVVGLYLTIMYIITKNMVYVNSRYVTAFHCLERDRVLLNAEKGDYCALTSLFLASENNA |
| | KYALGRNKRASIYIKHNCGTITKDFLVNYRNAIAHLSVVRNMESYISDIKYVDNYFALYHYTMQRW |
| | LFDQKAVTDQSPVFLKKYNNNLNEYHTYCKDFVKALNVPFAYNLARYKNLSIAELFDMNDTKTESS |
| | AKFGNEAIPVE |
| IMG_ | MGKGNHKSIAKASGLKSTFINGNEVTMTSFGKGNSAVLEKKIIDSEVEDLNPDKAFTVEENNVQKGK |
| 3300031992 | LRIKSNRMSDPATADNPVHVSPEKVGKSIKGQDIIGCKDVLEQRYFGQTFDDNIHIQLIYNILDIEKILA |
| SEQ ID NO: | VHVTNATFAVNNIMRIEDTENEDFIGNLSSCNTYDSFRNYESDTNLSPNVKANLKRSNEFFDIIKKDT |
| 5087 | RLGYFGPAFYEKKGKNFVRKPDKSIYHVLALIGNLRQFVVHDTTIITGKEKSRSWLYNIDKQIGPEFIQ |
| | TMTELYNAAVQNIDRDFIETNKVDIRIIHDAFYLIYGSSDWQKIAEEYYRFSIEKSYKNIGFSVKKLRE |
| | EIISTYAVKFENHKYDSVRHKLNKIIDFLIFTSYSTDDISQKVSVLRTCMNDEEKEERFYKPEAKNIWD |
| | KFRDIFNEFIPERINGKAVSELKKEHFSHREINIDSLKISRKNPDSFSKLIYLLTLFLDGKEINDLLTTLIN |
| | KFDNISSFISIMKEMNISCDFTDEYRFFNKSKYICSELRLINSFARMTSPVSLAKREMYREAVEILGTAG |
| | MNEDEKESLLDKVLCIDGNGKYISSKTDRNRDVNLRNFIANNVIESSRFKYLIRYNNAKKTRVLASN |
| | KTVVRFLLERIDELNEKQIDRYYETCSSDKTLKNKKDKIDFLTELIIKIDCSQFLKVKNRVRAGTAEA |
| | QEKERLKAVVGLYLTIMYIITKNMVYVNSRYVTAFHCLERDRVLLNAEKGDYCALTSLFLASENNA |
| | KYALGRNKRASIYIKHNCGTITKDFLVNYRNAIAHLSVVRNMESYISDIKYVDNYFALYHYTMQRW |
| | LFDQKAVTDQSPVFLKKYNNNLNEYHTYCKDFVKALNVPFAYNLARYKNLSIAELFDMNDTKTESS |
| | AKFGNEAIPVE |
| IMG_ | MGKGNHKSIAKASGLKSTFINGNEVTMTSFGKGNSAVLEKKIIDSEVEDLNPDKAFTVEENNVQKGK |
| 3300024342 | LRIKSNRMSDPATADNPVHVSPEKVGKSIKGQDIIGCKDVLEQRYFGQTFDDNIHIQLIYNILDIEKILA |
| SEQ ID NO: | VHVTNATFAVNNIMRIEDTENEDFIGNLSSCNTYDSFRNYESDTNLSPNVKANLKRSNEFFDIIKKDT |
| 5088 | RLGYFGPAFYEKKGKNFVRKPDKSIYHVLALIGNLRQFVVHDTTIITGKEKSRSWLYNIDKQIGPEFIQ |
| | TMTELYNAAVQNIDRDFIETNKVDIRIIHDAFYLIYGSSDWQKIAEEYYRFSIEKSYKNIGFSVKKLRE |
| | EIISTYAVKFENHKYDSVRHKLNKIIDFLIFTSYSTDDISQKVSVLRTCMNDEEKEERFYKPEAKNIWD |
| | KFRDIFNEFIPERINGKAVSELKKEHFSHREINIDSLKISRKNPDSFSKLIYLLTLFLDGKEINDLLTTLIN |
| | KFDNISSFISIMKEMNISCDFTDEYRFFNKSKYICSELRLINSFARMTSPVSLAKREMYREAVEILGTAG |
| | MNEDEKESLLDKVLCIDGNGKYISSKTDRNRDVNLRNFIANNVIESSRFKYLIRYNNAKKTRVLASN |
| | KTVVRFLLERIDELNEKQIDRYYETCSSDKTLKNKKDKIDFLTELIIKIDCSQFLKVKNRVRAGTAEA |
| | QEKERLKAVVGLYLTIMYIITKNMVYVNSRYVTAFHCLERDRVLLNAEKGDYCALTSLFLASENNA |
| | KYALGRNKRASIYIKHNCGTITKDFLVNYRNAIAHLSVVRNMESYISDIKYVDNYFALYHYTMQRW |
| | LFDQKAVTDQSPVFLKKYNNNLNEYHTYCKDFVKALNVPFAYNLARYKNLSIAELFDMNDTKTESS |
| | AKFGNEAIPVE |
| IMG_ | MGKGNHKSIAKASGLKSTFINGNEVTMTSFGKGNSAVLEKKIIDSEVEDLNPDKAFTVEENNVQKGK |
| 3300024342_2 | LRIKSNRMSDPATADNPVHVSPEKVGKSIKGQDIIGCKDVLEQRYFGQTFDDNIHIQLIYNILDIEKILA |
| SEQ ID NO: | VHVTNATFAVNNIMRIEDTENEDFIGNLSSCNTYDSFRNYESDTNLSPNVKANLKRSNEFFDIIKKDT |
| 5089 | RLGYFGPAFYEKKGKNFVRKPDKSIYHVLALIGNLRQFVVHDTTIITGKEKSRSWLYNIDKQIGPEFIQ |
| | TMTELYNAAVQNIDRDFIETNKVDIRIIHDAFYLIYGSSDWQKIAEEYYRFSIEKSYKNIGFSVKKLRE |
| | EIISTYAVKFENHKYDSVRHKLNKIIDFLIFTSYSTDDISQKVSVLRTCMNDEEKEERFYKPEAKNIWD |
| | KFRDIFNEFIPERINGKAVSELKKEHFSHREINIDSLKISRKNPDSFSKLIYLLTLFLDGKEINDLLTTLIN |
| | KFDNISSFISIMKEMNISCDFTDEYRFFNKSKYICSELRLINSFARMTSPVSLAKREMYREAVEILGTAG |
| | MNEDEKESLLDKVLCIDGNGKYISSKTDRNRDVNLRNFIANNVIESSRFKYLIRYNNAKKTRVLASN |
| | KTVVRFLLERIDELNEKQIDRYYETCSSDKTLKNKKDKIDFLTELIIKIDCSQFLKVKNRVRAGTAEA |
| | QEKERLKAVVGLYLTIMYIITKNMVYVNSRYVTAFHCLERDRVLLNAEKGDYCALTSLFLASENNA |
| | KYALGRNKRASIYIKHNCGTITKDFLVNYRNAIAHLSWRNMESYISDIKYVDNYFALYHYTMQRW |
| | LFDQKAVTDQSPVFLKKYNNNLNEYHTYCKDFVKALNVPFAYNLARYKNLSIAELFDMNDTKTESS |
| | AKFGNEAIPVE |
| IMG_ | MAKKKKAKQRREEQEAARMNKIQSAVKAKAETAPAVSSAFVEKRKDKQSKKTFAKASGLKSTLAV |
| 3300024270 | DNSAVMTVFGRGNEAKLDHRINADLQSESLHPQAALKNVHAPNKQKIHFIGRMQDMNLTADHPLH |
| SEQ ID NO: | SHDGERAVGADLLCAKDKLEQLYFGRTFNDNIHIQLIYQILDIQKILALHANNIIFALDNLLHKKNDE |
| 5090 | LSDDFVGMGRMRATIGYDAFRNSTNQKVQETYREFQEFVRRKELLYFGSAFYNGDTRRDEKVIYHI |
| | LSLAASVRQFCFHNDYTSDDGKGFIKADWMYRLEEALPAEYKDTLDALYLEGVEGLDQSFLKNNT |
| | VNIQILCSIFNHDDPNKIAEEYYGFLMTKEYKNMGFSIKKLRECMLELPELSGYKEDQYNSVRSKLY |
| | KLFDFIIAHYFRKHPEKGEEMVDCLRLCMTEDEKDSHYEGTAKKLVRELAYDMQEAAEQANGSNIT |
| | QMQKNEQQGKTKGMFAIRDEIRVSRKPVSYFSKVIYVMTLLLDGKEINDLLTTLINKFENIVSFEDVL |
| | RQLNVDCTFKPEFAFFG |
| IMG_ | MAKKKKAKQRREEQEAARMNKIQSAVKAKAETAPAVSSAFVEKRKDKQSKKTFAKASGLKSTLAV |
| 3300018495 | DNSAVMTVFGRGNEAKLDHRINADLQSESLHPQAALKNVHAPNKQKIHFIGRMQDMNLTADHPLH |
| SEQ ID NO: | SHDGERAVGADLLCAKDKLEQLYFGRTFNDNIHIQLIYQILDIQKILALHANNIIFALDNLLHKKNDE |
| 5091 | LSDDFVGMGRMRATIGYDAFRNSTNQKVQETYREFQEFVRRKELLYFGSAFYNGDTRRDEKVIYHI |
| | LSLAASVRQFCFHNDYTSDDGKGFIKADWMYRLEEALPAEYKDTLDALYLEGVEGLDQSFLKNNT |
| | VNIQILCSIFNHDDPNKIAEEYYGFLMTKEYKNMGFSIKKLRECMLELPELSGYKEDQYNSVRSKLY |
| | KLFDFIIAHYFRKHPEKGEEMVDCLRLCMTEDEKDSHYEGTAKKLVRELAYDMQEAAEQANGSNIT |
| | QMQKNEQQGKTKGMFAIRDEIRVSRKPVSYFSKVIYVMTLLLDGKEINDLLTTLINKFENIVSFEDVL |
| | RQLNVDCTFKPEFAFFGYDRCRNISGELRLINSFARMQKPSAKAKHVMYRDALRILGLDNGMSEEAL |
| | DQEVRRILQIGADGKPIKNANKGFRNFIASNVIESSRFRYLVRYNNPHKTRMIAQNEAIVRFVLSEIPD |
| | EQIRRYYDVCRDPKLPRSSSREAQVDILTGIITDVNYRIFEDVPQSKKINKDRPDANDRMTLKK |
| IMG_ | MAKKKKAKQRREEQEAARMNKIQSAVKAKAETAPAVSSAFVEKRKDKQSKKTFAKASGLKSTLAV |
| 3300022660 | DNSAVMTVFGRGNEAKLDHRINADLQSESLHPQAALKNVHAPNKQKIHFIGRMQDMNLTADHPLH |
| SEQ ID NO: | SHDGERAVGADLLCAKDKLEQLYFGRTFNDNIHIQLIYQILDIQKILALHANNIIFALDNLLHKKNDE |
| 5092 | LSDDFVGMGRMRATIGYDAFRNSTNQKVQETYREFQEFVRRKELLYFGSAFYNGDTRRDEKVIYHI |
| | LSLAASVRQFCFHNDYTSDDGKGFIKADWMYRLEEALPAEYKDTLDALYLEGVEGLDQSFLKNNT |
| | VNIQILCSIFNHDDPNKIAEEYYGFLMTKEYKNMGFSIKKLRECMLELPELSGYKEDQYNSVRSKLY |
| | KLFDFIIAHYFRKHPEKGEEMVDCLRLCMTEDEKDSHYEGTAKKLVRELAYDMQEAAEQANGSNIT |
| | QMQKNEQQGKTKGMFAIRDEIRVSRKPVSYFSKVIYVMTLLLDGKEINDLLTTLINKFENIVSFEDVL |
| | RQLNVDCTFKPEFAFFGYDRCRNISGELRLINSFARMQKPSAKAKHVMYRDALRILGLDNGMSE |
| GCA_ | MKKQKSKKTVSKTSGLKEALSVQGTVIMTSFGKGNMANLSYKIPSSQKPQNLNSSAGLKNVEVSGK |
| 002449585.1_ | KIKFQGRHPKIATTDNPLFKPQPGMDLLCLKDKLEMHYFGKTFDDNIHIQLIYQILDIEKILAVHVNNI |
| ASM244958v1_ | VFTLDNVLHPQKEELTEDFIGAGGWRINLDYQTLRGQTNKYDRFKNYIKRKELLYFGEAFYHENER |
| genomic | RYEEDIFAILTLLSALRQFCFHSDLSSDESDHVNSFWLYQLEDQLSDEFKETLSILWEEVTERIDSEFLK |
| SEQ ID NO: | TNTVNLHILCHVFPKESKETIVRAYYEFLIKKSFKNMGFSIKKLREIMLEQSDLKSFKEDKYNSVRAK |
| 5093 | LYKLFDFIITYYYDHHAFEKEALVSSLRSSLTEENKEEIYIKTARTLASALGADFKKAAADVNAKNIR |
| | DYQKKANDYRISFEDIKIGNTGIGYFSELIYMLTLLLDGKEINDLLTTLINKFDNIISFIDILKKLNLEFK |
| | FKPEYADFFNMTNCRYTLEELRVINSIARMQKPSADARKIMYRDALRILGMDNRPDEEIDRELERTM |
| | PVGADGKFIKGKQGFRNFIASNVIESSRFHYLVRYNNPHKTRTLVKNPNVVKFVLEGIPETQIKRYFD |
| | VCKGQEIPPTSDKSAQIDVLARIISSVDYKIFEDVPQSAKINKDDPSRNFSDALKKQRYQAIVSLYLTV |
| | MYLITKNLVYVNSRYVIAFHCLERDAFLHGVTLPKMNKKIVYSQLTTHLLTDKNYTTYGHLKNQKG |
| | HRKWYVLVKNNLQNSDITAVSSFRNIVAHISVVRNSNEYISGIGELHSYFELYHYLVQSMIAKNNWY |
| | DTSHQPKTAEYLNNLKKHHTYCKDFVKAYCIPFGYVVPRYKNLTINELFDRNNPNPEPKEEV |
| IMG_ | MYRDALRILGLDNGMSEEALDQEVRRILQIGADGKPIKNANKGFRNFIASNVIESSRFRYLVRYNNPH |
| 3300024270_2 | KTRMIAQNEAIVRFVLSEIPDEQIRRYYDVCRDPKLPRSSSREAQVDILTGIITDVNYRIFEDVPQSKKI |
| SEQ ID NO: | NKDRPDANDRMTLKKQRYQAIVSLYLTVMYLVTKNLVYVNSRYVMAFHALERDAYLYGITNIKGD |
| 5094 | YRKLTDNLLADENYKKFGHFKNKKWRGIAEQNLRNSDVPVIKSFRNMAAHISVIRNIDLYIGDIQKV |
| | DSYFALYHFLMQKLIQRVVPENTKGLSDQTKKYYDALEQYNTYCKDFVKAYCTPFAYVTPRYKNL |
| | TIDGLFDRNRPGEDK |
| OWQH01.1 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5095 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| OVZD01.1_2 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5096 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| GCA_ | MGKKIHARDLREQRKTDRTEKFADQNKKREAERAVQKKDAAVSVKSVSSVSSKKDNVTKSMAKA |
| 003524315.1_ | AGVKSVFAVGNTVYMTSFGRGNDAVLEQKIVDTSHEPLNIDDPAYQLNVVTMNGYSVTGHRGETV |
| ASM352431v1_ | SAVTDNPLRRFNGGKKDEPEQSVPTDMLCLKPTLEKKFFGKEFNDNIHIQLIYNILDIEKILAVYSTNA |
| genomic | VYALNNTIADENNENWDLFANFSTDNTYDELNAIATYKKSADDVSTDDEKRREAEKKKREAKIAEK |
| SEQ ID NO: | ILADYEKFRKNNRLAYFADAFYVDKNKSKSKPKDKAKGIQREKKKFILYLL |
| 5097 | |
| UEOK01.1_2 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5098 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| OWSW01.1 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5099 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| OZRL01.1 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5100 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| OGJH01.1 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5101 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| UPRD01.1_2 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5102 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| OWRJ01.1_2 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5103 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| OKRY01.1 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5104 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| OWEZ01.1_2 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5105 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| OGYF01.1 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5106 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| UPEW01.1_2 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5107 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| UAPH01.1 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5108 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| UAPL01.1 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5109 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| UBLP01.1 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5110 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| UZQR01.1 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5111 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| OGXR01.1_2 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5112 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| UZKF01.1_2 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5113 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| OHAO01.1 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5114 | SAVTDNPLRRFNGGKKDEPEQSVPTDMLCLKPTLEKKFFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| UZOF01.1 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5115 | SAVTDNPLRRFNGGKKDEPEQSVPTDMLCLKPTLEKKIFGKEFNDNIHIQLIYNILDIEKILAVYSTNA |
| | VYALNNTIADENNENWDLFANFSTDNTYDELNAIATYKKSADDVSTDDEKRREAEKKKREAKIAEK |
| | ILADYEKFRKNNRLAYFADAFYVDKNKSKSKPKDKAKGIQREKKKFILYLL |
| mgm4743569.3 | MXXXXXXXXXXXXXXXXXXXXXXXXXXXPVMGKKIHARDLREQRKTDRTVKFADQNKKREAE |
| SEQ ID NO: | RAVQKKDAAVSVKSVPSVSSKKDNVTKSMAKAAGVKSVFAVGNTVYMTSFGRGNDAVLEQKIVD |
| 5116 | TSHEPLNIDDPAYQLNVVTMNGYSVIGHRGETVSAVTDNPLRRFNGGKKDEPEQSVPTDMLCLKPT |
| | LEKKFFGKEFDDNIHIQLIYNILDIEKILAVYSTNAIYALNNTIADENDENWDLFANFSTDNTYYELRN |
| | AAAYKESADDESTDDEKRREAEKKKREAKKAEKILADYEKFRKNNRLAYFADAFYVDKNKSKSKS |
| | KDKAEGIQRGKKEIYSILALIAKLRHWCVHSEDGRAEFWLYKLDELKDDFKNVLDVVYNRPVEEIN |
| | NRFIENNKVNIQILDSVYENTDIAELTRSYYEFLITKKYKNMGFSIKKLREIILEGTEYNDNKYDTVRN |
| | KLYQIVDFILYRGYINENSERAEVLVNALRSTLNEDDKTKLYSSEAAFLKKKYMKXXXXXXXXXXX |
| | XXXXXIYENYQKISRFA |
| OROX01.1 | MLCLKPTLEKKFFGKEFNDNIHIQLIYNILDIEKILAVYSTNAIYALNNMSADENIENSDFFMKRTTDE |
| SEQ ID NO: | TFDDFEKKKESTNSREKADFDAFEKFIGNYRLAYFADAFYVDKNKSKSKPKDKAKGIQRGEKEIYSI |
| 5117 | LALIAKLRHWCVHSEEGRAEFWLYKLDELKSDFKNVLDVVYNRPVEKINNRFIENNKVNIQILGSVY |
| | KNTDIAELVRSYYEFLITKKYKNMGFSIKKLRESMLEGKGYADKEYDSVRNKLYQMTDFILYTGYIN |
| | EDSDRADDLVNTLRSSLKEDDKTTVYCKEADYLWKKYRESIREVAASLDVKNINELKNNAFTIPDN |
| | ELRKCFISYADSVSEFTKLIYLLTRFLSGKEINDLVTTLINKFDNIRSFLEVMDELGLERTFTDEYSFFE |
| | GSTKYLAELVELNSFVKSCSFDINAKRTMYRDALDILGIESGKTEEDIEKMIDNIVQFDANGKKLPNK |
| | NHGLRNFIASNVIDSNRFEYLVRYGNPKKIRETAKCKPAVRFVLNEIPDAQIERYYKACYPDEKSLCF |
| | ANMQRDKLAGVIANIKFDDFSDAGSYQKANATSTKITSEAEIKRKNQAIIRLYLTVMYIMLKNLVNV |
| | NARYVIAFHCVERDTKLYAESGLEVGNIEKNKTNLTMAVMGVKLENGIIKTEFDKSLAENAANRYF |
| | RNARWYKLILDNLKKSERAVVRYFVIIYLKSADLP |
| ULZQ01.1 | MLCLKPTLEKKFFGKEFNDNIHIQLIYNILDIEKILAVYSTNAIYALNNMSADENIENSDFFMKRTTDE |
| SEQ ID NO: | TFDDVEKKKESTNSREKADFDAFEKFIGNYRLAYFADAFYVDKNKSKSKPKDKAKGIQRGEKEIYSI |
| 5118 | LALIAKLRHWCVHSEEGRAEFWLYKLDELKSDFKNVLDVVYNRPVEKINNRFIENNKVNIQILGSVY |
| | KNTDIAELVRSYYEFLITKKYKNMGFSIKKLRESMLEGKGYADKEYDSVRNKLYQMTDFILYTGYIN |
| | EDSDRADDLVNTLRSSLKEDDKTTVYCKEADYLWKKYRESIREVAASLDVKNINELKNNAFTIPDN |
| | ELRKCFISYADSVSEFTKLIYLLTRFLSGKEINDLVTTLINKFDNIRSFLEVMDELGLERTFTDEYSFFE |
| | GSTKYLAELVELNSFVKSCSFDINAKRTMYRDALDILGIESGKTEEDIEKMIDNIVQFDANGKKLPNK |
| | NHGLRNFIASNVIDSNRFEYLVRYGNPKKIRETAKCKPAVRFVLNEIPDAQIERYYKACYPDEKSLCF |
| | ANMQRDKLAGVIANIKFDDFSDAGSYQKANATSTKITSEAEIKRKNQAIIRLYLTVMYIMLKNLVNV |
| | NARYVIAFHCVERDTKLYAESGLEVGNIEKNKTNLTMAVMGVKLENGIIKTEFDKSLAENAANRYF |
| | RNARWYKLILDNLKKSERAVVRYFVIIYLKSADLP |
| ORPS01.1 | LIHALNNMSADENIENSDFFMKRTTDETFDDFEKKKESTNSREKADFDAFEKFIGNYRLAYFADAFY |
| SEQ ID NO: | VDKNKSKSKPKDKAKGIQRGEKEIYSILALIAKLRHWCVHSEEGRAEFWLYKLDELKSDFKNVLDV |
| 5119 | VYNRPVEKINNRFIENNKVNIQILGSVYKNTDIAELVRSYYEFLITKKYKNMGFSIKKLRESMLEGKG |
| | YADKEYDSVRNKLYQMTDFILYTGYINEDSDRADDLVNTLRSSLKEDDKTTVYCKEADYLWKKYR |
| | ESIREVAASLDVKNINELKNNAFTIPDNELRKCFISYADSVSEFTKLIYLLTRFLSGK |
| USZB01.1 | LEHIALCAGVAVVFLALSLFDNYRTCAITEKYTACSVRIVYLTCQTLAGYNQHRLVGIVCKEVSCNV |
| SEQ ID NO: | NRCYKACAGSIYIHAGTGLCADQILYKAGCVRIIEVGRAGRNYDAVDTSHEPLNIDDPAYQLNVVT |
| 5120 | MNGYSVTGHRGETVSAVTDNPLRRFNGGKKDEPEQSVPTDMLCLKPTLEKKFFGKEFDDNIHIQLIY |
| | NILDIEKILAVYSTNAIYALNNMSADENIENSDFFMKRTTDETFDDFEKKKESTNSREKADFDAFEKFI |
| | GNYRLAYFADAFYVNKKNPKGKARNVLREDKELYSVLTLIGKLRHWCVHSEEGRAEFWLYKLDEL |
| | KDDFKNVLDVVYNRPVEEINNRFIENNKVNIQILGSVYKNTDIAELVRSYYEFLITKKYKNMGFSIKK |
| | LRESMLEGKGYADKEYDSVRNKLYQMTDFILYTGYINEDSDRADDLVNTLRSSLKEDDKTTVYCKE |
| | ADYLWKKYRESIREVADALDGDNIKRLSKSNIEIQEDKLRKCFISYADSVSEFTKLIYLLTRFLSGKEI |
| | NDLVTTLINKFDNIRSFLEIMDELGLDRTFTAEYSFFEDSTKYLAELVELNSFVKSCSFDINAKRTMYR |
| | DALDILGIKSGKTEEDIEKMIDNILQIDANGDKKLKKNNGLRNFIASNVIDSNRFKYLVRYGNPKKIRE |
| | TAKCKPAVRFVLNEIPDAQIERYYEACCPENTALCSANKKREKLADMIAEIEFENFSDAGNYQKANV |
| | TSKTHEAEIKRKNQSIIRLYLTVMYIMLKNLVNVNARYVIAFHCVERDTKLYAESGLEVGNIEKNKT |
| | NLTMAVMGVKLENGIIKTEFDKSFADENAANRYLRNARWYKLILDNLKKSERAVVNEFRNTVCHLN |
| | AIRNI |
| IMG_ | MGKKVHARDLRNQKVLEQKAKYAKQNIEREAQKAVQKNEVSSTAKSANAVLSENNKTGKSKAKA |
| 3300010266 | AGVKSVFAVGNTVYMTSFGRGNDAVLEQKIIDTSHQPLNVDDPAYKLTDVTVTSYFVEGHRGETVS |
| SEQ ID NO: | AVTDNPLCRFNGKKHRDENQGEPVQSVPTDMLCLKSSLEKKFFGKEFNDNIHIQLIYNILDIEKILAV |
| 5121 | YSTNAIYALNNMSADKNVENSDFFMKRTTDETFDDFEKKKESTNSREKADFDAFEKFIGNYRLAYF |
| | ADAFYVNKKNPKGKVRNVLREDKELYSVLTLIGKLRHWCVHSEEGRAEFWLYRLDELKSDFKNVL |
| | DSVYNRPVEEINDDFVERNKVNIQILHSIYENTDIAELVRSYYEFLITKKYKNMGFSIKKLRESMLEGK |
| | GYADKEYDSVRNKLYQMTDFILYTGYINEDSDRANDLVNTLRSSLKEDDKTTVYCKEADYLWEKY |
| | RKSIKEVADALDGDNIKRLSKSNIEIQEDELRKCFIGYADSVSEFTKLIYLMTRFLSGKEINDLVTTLIN |
| | KFDNIRSFLEVMDELGLERTFTDEYRFFEGSTKYLAELVELNSFVKSCSFDINAKRTMYRDALDILGI |
| | KSGKTEEDIEKMIDNILQIDANGDKKLKKNNGLRNFIASNVTDSNRFKYLVRYGNPKKIRETAKCKPA |
| | VRFVLNEIPDAQIERYYEACCPENTALCSANKRREKLADMIAEIEFENFSDAGNYQKANVTSKTHEA |
| | EIKRKNQSIIRLYLTVMYIMLKNLVNVNDRYVIAFHCVERDTKLYVESGLEVGNIEKNKTNLTMAV |
| | MGVKLENGIIKTEFDKSLAENAANRYLRNARWYKLILDNLKK |
| ORCP01.1 | MGKKIHARDLREQRKTDRTEKFADQNKKREAERAVQKKDAAVSVKSVSSVSSKKDNVTKSMAKA |
| SEQ ID NO: | AGVKSVFAVGNTVYMTSFGRGNDAVLEQKIVDTSHEPLNIDDPAYQLNVVTMNGYSVTGHRGETV |
| 5122 | SAVTDNPLRRFNGGKKDEPEQSVPTDMLCLKPTLEKKFFGKEFNDNIHIQLIYNILDIEKILAVYSTNA |
| | IYALNNMSADENIENSDFFMKRTTDETFDDFEKKKESTNSREKADFDAFEKFIGNYRLAYFADAFYV |
| | NKKNPKGKARNVLREDKELYSVLTLIGKLRHWCVHSEEGRAEFWLYKLDELKDDFKNVLDVVYNR |
| | PVEEINNRFIENNKVNIQILGSVYKNTDIAELVRSYYEFLITKKYKNMGFSIKKLRESMLEGKGYADK |
| | EYDSVRNKLYQMTDFILYTGYINEDSDRADDLVNTLRSSLKEDDKTTVYCKEADYLWKKYRESIRE |
| | VADALDGDNIKRLSKSNIEIQEDKLRKCFISYADSVSEFTKLrYLLTRFLSGKEINDLVTTLINKFDNIR |
| | SFLEIMDELGLDRTFTAEYSFFEGSTKYLAELVELNSFVKSCSFDINAKRTMYRDALDILGIKSGKTEE |
| | DIEKMIDNILQIDANGDKKLKKNNGLRNFIASNVTDSNRFKYLVRYGNPKKIRETAKCKPAVRFVLNE |
| | IPDAQIERYYEACCPKNTALCSANKRREKLADMIAEIEFENFSDAGNYQKANVTSRTSEAEIKRKNQ |
| | AHRLYLTVMYIMLKNLVNVNARYVIAFHCVERDTKLYAESGLEVGNIEKNKRHIVKNKLLNKHKA |
| | DLVCRRQINDTGK |
| IMG_ | MGKKIHARDLRERRKTDRTEKFADQNKKREAERAVQKKDAAVSVKSVSSVSSKKDNVTKSMAKA |
| 3300006464 | AGVKSVFAVGNTVYMTSFGRGNDAVLEQKIVDTSHEPLNIDDPAYQLNVVTMNGYSVTGHRGETV |
| SEQ ID NO: | SAVTDNPLRRFNGGKKDEPEQSVPTDMLCLKPTLEKKFFGKEFDDNIHIQLIYNILDIEKILAVYSTNA |
| 5123 | IYALNNMSADENIESSDFFMKRTTDETFDDFEKKKESTNSREKADFDAFEKFIGNYRLAYFADAFYV |
| | NKKNPKGKARNVLREDKELYSVLTLIGKLRHWCVHSEEGRAEFWLYKLDELKDDFKNVLDVVYNR |
| | PVEEINNRFIENNKVNIQILGSVYKNTDIAELVRSYYEFLITKKYKNMGFSIKKLRESMLEGKGYADK |
| | EYDSVRNKLYQMTDFILYTGYINEDSDRADDLVNTLRSSLKEDDKTTVYCKEADYLWKKYRESIRE |
| | VADALDGDNIKRLSKSNIEIQEDKLRKCFISYADSVSEFTKLIYLLTRFLSGKEINDLVTTLINKFDNIR |
| | SFLEIMDELGLDRTFTAEYSFFEGSTKYLAELVELNSFVKSCSFDINAKRTMYRDALDILGIKSGKTEE |
| | DIEKMIDNILQIDANGDKKLKKNNGLRNFIASNVTDSNRFKYLVRYGNPKKIRETAKCKPAVRFVLNE |
| | IPDAQIERYYEACCPKNTALCSANKRREKLADMIAEIEFENFSDAGNYQKANVTSRTSEAEIKRKNQ |
| | AIIRLYLTVMYIMLKNLVNVNARYVIAFHCVERDTKLYAESGLEVGNIEKNKTNLTMAVMGVKIEN |
| | GIIKTEFDKSLAENAANRYLRNARWYKLILDNLKKSERAVVNEFRNTVCHLNAIRNININSDGIKEVE |
| | NYVALYHY |
| OYAG01.1 | MGKKIHARDLREQRKTDRTEKFADQNKKREAERAVQKKDAAVSVKSVSSVSSKKDNATKSMAKA |
| SEQ ID NO: | AGVKSVFAVGNTVYMTSFGRGNDAVLEQKIVDTSHEPLNIDDPAYQLNVVTMNGYSVTGHRGETV |
| 5124 | SAVTDNPLRRFNGGKKDEPEQSVPTDMLCLKPTLEKKFFGKEFDDNIHIQLIYNILDIEKILAVYSTNA |
| | IYALNNMSADENIENSDFFMKRTTDETFDDFEKKKESTNSREKADFDAFEKFIANYRLAYFADAFYV |
| | NKKNPKGKARNVLREDKELYSVLTLIGKLRHWCVHSEEGRAEFWLYKLDELKDDFKNVLDVVYNR |
| | PVEEINNRFIENNKVNIQILGSVYKNTDIAELVRSYYEFLITKKYKNMGFSIKKLRESMLEGKGYADK |
| | EYDSVRNKLYQMTDFILYTGYINEDSDRADDLVNTLRSSLKEDDKTTVYCKEADYLWKKYCESIRE |
| | VAEALDGDNIKRLSKSNIEIRDNELRKCFISYADSVSEFTKLIYLLTRFLSGKEINDLVTTLINKFDNIRS |
| | FLEIMDELGLDRTFTAEYSFFEDSTKYLAELVELNSFVKSCSFDINAKRTMYRDALDILGIKSGKTEED |
| | IEKMIDNILQIDANGDKKLKKNNGLRNFIASNVTDSNRFKYLVRYGNPKKIRETAKCKPAVRFVLNEI |
| | PDAQIERYYEACCPKNTALCSANKRREKLADMIAEIKFENFSDAGNYQKANVTSKTHEAEIKRKNQ |
| | AHRLYLTCLLYTSPSPRD |
| OWQH01.1_2 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5125 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| OVZD01.1 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5126 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| UEOK01.1 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5127 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| OWSW01.1_2 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5128 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| OZRL01.1_2 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5129 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| OYBO01.1 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5130 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| OGJH01.1_2 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5131 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| UPRD01.1 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5132 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| OWRJ01.13 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5133 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| OKRY01.1_2 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5134 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| OWEZ01.1 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5135 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| UZOF01.1_2 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5136 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| OGYF01.12 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5137 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| UPEW01.1 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5138 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| UAPH01.1_2 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5139 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| UAPL01.1_2 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5140 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| UBLP01.1_2 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5141 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| UZQR01.1_2 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5142 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| OGXR01.1 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5143 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADIKFDDFSDAGS |
| | YQKANATSTRRTSEAEIKRKNQAIIRLYLTVMYIMLKNLVNVNARYVIAFHCLERDAKLYSESVLKV |
| | GNTNEESRLQTGNTNEEKNKVKLTNLTNLTMAVMGVKLENGTIKTEFDKSLAENAANRYLRNARW |
| | YKLILDNLKKSERAVVTEFRNTVCHLNAIRNININIKEVKEVENYFALYHYLIQKHLENRFADKKVER |
| | DTGDFISKLEEHKTYCKDFVKAYCTPFGYNLVRYKNLTIDGLFDKNYPGKDDSDKQK |
| UZKF01.1 | LYKLDELKDDFKNVLDVVYNRPVEEINNRFIENNKVNIQILGSVHEDTDIAELTRSYYEFLITKKYKN |
| SEQ ID NO: | MGFSIKKLREIILEGTEYNDNKYDTVRNKLYQIVDFILYRGYINENSERAEVLVNALRSTLNEDDKTK |
| 5144 | LYSSEAAFLKKKYMKIIRKAADSLDVKKLKDLKKKAFTIPDNELRKCFISYADSVSEFTKLIYLLTRFL |
| | SGKEINDLVTTLINKFDNIRSFLEIMDELGLERTFTDEYRFFEGSTKYLAELVELNSFVKSCSFDMSAK |
| | RTMYRDALDILGIESDKTEEDIEKMIDNILQVDANGKKLPNKNHGLRNFIASNVIDSNRFEYLVRYGN |
| | PKKIRETAKCEPAVRFVLNEIPDAQIERYYKAYYPDEKSLCLANMQRDKLAGVIADISDAGSYQKAN |
| | ATSTRRTSEAEIKRKNQAIIRLYLTVMYIMLKNLVNVNARYVIAFHCLERDAKLYSESVLKVGNTNE |
| | ESRLQTGNTNEEKNKVKLTNLTNLTMAVMGVKLENGTIKTEFDKSLAENAANRYLRNARWYKLIL |
| | DNLKKSERAVVTEFRNTVCHLNAIRNININIKEVKEVENYFALYHYLIQKHLENRFADKKVERDTGD |
| | FISKLEEHKTYCKDFVKAYCTPFGYNLVRYKNLTIDGLFDKNYPGKDDSDKQK |
| ORTQ01.1 | LAKAAGLKSTFVISPQEKELAMTAFGRGNDALLQKRIVDGVVRDVAGEKQQFQVQRQDESRFRLQN |
| SEQ ID NO: | SRLADRTVTADDPLHRAETPRRQPLGAGMDQLRRKAILEQKYFGRTFDDNIHIQLIYNILDIHKMLA |
| 5145 | VPANHIVHTLNLLGGYGETDFMGMLPAGLPYDKLRVVKKKNGDTVDIKADIAAYAKRPQLAYLGA |
| | AFYDVTPGKSKRDAARGRVKREQDVYTILSLMSLLRQFCAHDSVRIWGQNTPAALYHLQALPQDM |
| | KDLLDDSWRRALGGVNDHFLDTNKVNLLTLFEYYGAETKQARVALTQDFYRFVVLKEQKNMGFSL |
| | RRLREELLKLPDAAYLTGQEYDSVRQKLYMLLDFLLCRLYAQERADRCEELVSALRCALSDEEKDT |
| | VYQAEAAALWQALGDTLRRELLPLLKGKKLQDKDKKKPDELGLSRDVLDGVLFRPAQQGNRANA |
| | DYFCRLMHLSTWFMDGKEINTLLTTLISKFENIDSLRNVLESMGLACSFVPAYAMFDHSRYIAGQLR |
| | VVNNIARMRKPAIGAKREMYRAAVVLLGVDSPEAAAAITDDLLQIDPETGKVRPRGDSARDTGLRN |
| | FIANNVVESRRFTYLLRYMTPEQARVLAQNEKLIAFVLSTVPDTQLERYCRTCGREDITDRPAQIRYL |
| | TAQIMGVRYESFTDVEQRGRGDNPKKERYKALIGLYLTVLYLAVKNMVNCNARYVIAFYCRDRDT |
| | ALYQKEVCWYDLEEDKKSGKQRQVEDYTALTRYFVSQGYLNRHACGYLRSNMNGIGNGLLTAYR |
| | NAVDHLNVIPPLGSLCRDIGRVDSYFALYHYAVQQYLNGRYYRKTPREQELFAAMAQHRTWCSDL |
| | VKALNTPFGYNLARYKNLSIDGLFDREGDHVVREDGEKPAE |
| UMHX01.1 | LAKAAGLKSTFVISPQEKELAMTAFGRGNDALLQKRIVDGVVRDVAGEKQQFQVQRQDESRFRLQN |
| SEQ ID NO: | SRLADRTVTADDPLHRAETPRRQPLGAGMDQLRRKAILEQKYFGRTFDDNIHIQLIYNILDIHKMLA |
| 5146 | VPANHIVHTLNLLGGYGETDFMGMLPAGLPYDKLRVVKKKNGDTVDIKADIAAYAKRPQLAYLGA |
| | AFYDVTPGKSKRDAARGRVKREQDVYTILSLMSLLRQFCAHDSVRIWGQNTPAALYHLQALPQDM |
| | KDLLDDSWRRALGGVNDHFLDTNKVNLLTLFEYYGAETKQARVALTQDFYRFVVLKEQKNMGFSL |
| | RRLREELLKLPDAAYLTGQEYDSVRQKLYMLLDFLLCRLYAQERADRCEELVSALRCALSDEEKDT |
| | VYQAEAAALWQALGDTLRRELLPLLKGKKLQDKDKKKPDELGLSRDVLDGVLFRPAQQGNRANA |
| | DYFCRLMHLSTWFMDGKEINTLLTTLISKFENIDSLRNVLESMGLACSFVPAYAMFDHSRYIAGQLR |
| | VVNNIARMRKPAIGAKREMYRAAVVLLGVDSPEAAAAITDDLLQIDPETGKVRPRGDSARDTGLRN |
| | FIANNVVESRRFTYLLRYMTPEQARVLAQNEKLIAFVLSTVPDTQLERYCRTCGREDITDRPAQIRYL |
| | TAQIMGVRYESFTDVEQRGRGDNPKKERYKALIGLYLTVLYLAVKNMVNCNARYVIAFYCRDRDT |
| | ALYQKEVCWYDLEEDKKSGKQRQVEDYTALTRYFVSQGYLNRHACGYLRSNMNGIGNGLLTAYR |
| | NAVDHLNVIPPLGSLCRDIGRVDSYFALYHYAVQQYLNGRYYRKTPREQELFAAMAQHRTWCSDL |
| | VKALNTPFGYNLARYKNLSIDGLFDREGDHVVREDGEKPAE |
| ORMU01.1 | LKRMLLCAIIQASTKRDRKGGVRMEREVKKPPKKSLAKAAGLKSTFVISPQEKELAMTAFGRGNDA |
| SEQ ID NO: | LLQKRIVDGVVRDVAGEKQQFQVQRQDESRFRLQNSRLADRTVTADDPLHRAEAPHRQPLGAGMD |
| 5147 | QLRRKAVLEQKYFGRTFDDNIHIQLIYNILDIHKMLAVPANHIVHTLNLLGGYGETDFVGMLPAGLP |
| | YDKLWTPKAGDTSRNVKRDVKADIAAYAKRPQLAYLGAAFYDVTPGKSKRDAARGRVKREQDVY |
| | TILSLMSLLRQFCAHDSVRIWGQNTPAALYHLQALPQDMKDLLDDGWRRALGGVNDHFLDTNKVN |
| | LLTLFEYYGAETKQERVALTQDFYRFVVLKEQKNMGFSLRRLREELLKLPDAAYLTGQEYDSVRQK |
| | LYMLLDFLLCRLYAQERADRCEELVSALRCALSDEEKDAVYQAEAAALWQALGDTLRRELL |
| OQVZ01.1 | MLPFFRPQRRTTVMTAFGQGSTAILEKQIVDRAISDLQPVQQFQVEPASAAKYRLKNSRVRFPDVTA |
| SEQ ID NO: | DDPLYRRKDGGFVPGMDALRRKNVLEQRFFGRSFADNIHIQMIYSILDIHKILAAASGHIVHLLNIVK |
| 5148 | GDADRDFLGMLAAHVSYDKLNEKAEKSIADFCKSPRLIYYSAAFYETLDNGKSERRSNEDIFNILAL |
| | MTCLRNFSSHHSIAIKVRDYSAAGLYNLRRLGTDMKKMLDTFYTEAFRQLNRSFQDHNTTNLTCLF |
| | DILNISDSARQKQLAEEFYRYVVFKEQKNLGFSVRKLREEMLLLPDAAVIADKRYDTCRSKLYNLM |
| | DFLILRVYRTGRADRCDKLTEALRAALTDEEKAAVYHEEALSLWNEMRTLILDGLLPQMTPENLSR |
| | LSGQKRKGELSLDGAMLKECLYEPGPVPEDAAPEEANAEYFCRMIYLATLFMDGKEINTLLTTLISK |
| | FENIAAFLQTMEQMNIEAELGPEYAIFTRSRAVAEQLRVINSFALMKKPQVNAKQQLYRAAVTLLGT |
| | EDPDGVTDEMLRIDPVTGKMLPLNRRHHGDTGLRNFIANNVVESRRFQYLIRYSDPAQLHQLASNK |
| | KLVRFVLSGIPDTQINRYYETCGQTRPAGRAAKVEFLTGMIAAIRFDQFRDVNQKERGANTQKERYK |
| | AMLGLYQTVLYLAVKNLVNINARYVMAFHCVERDMFLYDGELTDPKGSNVPAFLAVNGRDGVQP |
| | QYLLLTQLFIQRGYLKRSACEQIQHNMTNISDRLLREYRNAVAHLNVIAHLADYSADMREITSYYGL |
| | YHYLMQRHLFKRHAWQVRRPERPTEEEEKRIEQEQKQLAWERALFDKTLQYHSYNKDLVKALNAP |
| | FGYNLARYKNLSIEQLFSKDAAPATEIKANRA |
| IMG_ | MAKKLNAKEKRLLSQQAQKDKYKKMEEERQAAEQARLAEEARQKAETERKKKEALEKEAREKLE |
| 3300022661 | KELLEKNPSLKKVKKSKAKAAGLKSTFAIGNELLMTSFGKGNEAVPEHYIFENGMISMQNPPAFDVE |
| SEQ ID NO: | NRGTSFSIAGKNAMNAIADDPRRTSKKVIGEDLIGAKSALEKRYFGKTFEDNIHIQLIYNILDIEKILSK |
| 5149 | HINNIVFSINNIRGIADPERDDLIGYMSLRDSYEYFMDEKNEKRKVLRESYRKLFKSSRLSYFGSAFYV |
| | EKQSTQKKKSGQKNFELRNEKDCYYMLALLGELRQVLAHSSAQKQGKEQTEARSSVYNLRTVGNN |
| | DMRKILNSLYAERVDALNENFMNYAKKDLTILFGIMDASSYEKKAEIARDFYDFTVRKSYKNIGFSI |
| | KKIRERMMDCFFANVKEITPDSPDFQSVRQKFYKILDFLLFTYYKDNKEETKKIIAALRASLTELEKE |
| | LFYQKQANILWNKMQMVVTEQIYPRTKGSAIQNLIADPEISADMLKGVMITPESVDYFSKMIYLLTL |
| | FIDGKEINDLLTTMINKFENIASFVSVLKDRGLPCNFRNDYSVFNNALKISAELREINSFARMEKATPG |
| | AKKQMFLDAAIILGSNESAEELS |
| OHBM01.1 | VKKIPQLAYFGSAFYNTPEDTSAKITKTKIKSNEEIYYTFMLLSTARNFSAHYLDRNRAKSSDAEDFD |
| SEQ ID NO: | GTSVIMYNLDNEELYKKLYNKKVHMALTGMKKVLDANFNKKVEHLNNSFIKNSAKDFVILCEVLGI |
| 5150 | KSRDEKTKFVKDYYDFVVRKNYKHLGFSVKELRELLFANHDSNKYIKEFDKISNKKFDSVRSRLNRL |
| | ADYIIYDYYNKNNAKVSDLVKYLRAAADDEQKKKIYLNESINLVKSGILERIKKILPKLNGKIIGNMQ |
| | PDSTITASMLHNTGKDWHPISENAHYFTKWIYTLTLFMDGKEINDLVTTLINKFDNIASFIEVLKSQSV |
| | CTHFSEERKMFIDSAEICSELSAMNSFARMEAPGASSKRAMFVEAARILGDNRSKEELEEYFDTLFDK |
| | SASKKEKGFRNFIRNNVVDSNRFKYLTRYTDTSSVKAFSNNKALVKFAIKDIPQEQILRYYNSCFGAS |
| | ERYYNDGMSDKLVEAIGKINLMQFNGVIQQADRNMLPEEKKKANAQKEKYKSIIRLYLTVCYLFFK |
| | NLVYVNSRYYSAFYNLEKDRSLFEINGELKPTGKFDEGHYTGLVKLFIDNGWINPRASAYLTVNLAN |
| | SDETAIRTFRNTAEHLEALRNADKYLNDLKQFDSYFEIYHYITQRNIKEKCEMLKEQTVKYNNDLLK |
| | YHGYSKDFVKALCVPFGYNLPRFKNLSIDALFDKNDKREKLKKGFED |
| UXMD01.1 | MEGINMGRKVHFAKAAGLKSAFAIGNDKVIVTSFGKGNDAILEKTIENDKVINIEQNIDIELMKRDFN |
| SEQ ID NO: | VKRRGDHGRPIITNNPGNREKIGKDMIDRKDQLEMRYFGRTFDDNIHIQLIYNIMDIEKILSIHVNNAL |
| 5151 | YALNNVLNRGSGDFNDTIGMMLAKPYDVFRNSEKYANFNENUCKPQLSYFGGAFFETGFNTKAQKR |
| | KTEKKSEKDIYYIISLLGFIRQAAVHGYDWNKTDEAAVALYTLDESFDKLYKNTKFRQEARRALDNL |
| | YDTRIDALNSQFLENASKDLTIIFKIYSVTDRSSKIKLIRQYYDFVVKKEYKYLGFSIKQLREMIADDN |
| | SIIKSKNYDTMRARLNRLIDFVIYINYIENPEKAKKLVENLRSHIGDDEKSRIYASEAKQLWQLLKGPV |
| | MDGILPQMNGKTISSMRADTVISETSVERMKNKTWEPIGKSAHYFIKLVYLLTLFLDGKEINDLVTTL |
| | INKMDNISSFNKLLENIDSKPPYEEEYVIFADSVIIADDLRVLNSFARMEKPDASAKRIMFVEAAQML |
| | GDKGTEQELTKYFDDLMDRNSSKEQKGFRNFIRNNVIESSRFKYLVRYCSVEDVIKFSKNKALIKFVL |
| | KEIPENQILRYYNSCTGNECREFSSEMTLKLAELIENMSFEQFENVRQNARRNSSEETDKLQKQNIIRL |
| | YLTVCYIFFKNLIYVNSRYFLAFYCLERDLRLWDIFENEQSYLMLTEKFMELGKVRKTAKKSKVKNS |
| | GEPSYEDAKHIPYNYIAANLRNADNVAIRKFRNITDHIITVQEAGLYLQDIKNPESYYQIYHYIAQRNL |
| | CDKLKNSNITEKTKEYFALVKNHGSYCKDFVKALCVPFGYNLPRFKNLSIDGLFDMNDKRENRDTT |
| | TEG |
| GCA_ | MEGINMGRKVHFAKAAGLKSAFAIGNDKVIVTSFGKGNDAILEKTIENDKVINIEQNIDIELMKRDFN |
| 900555525.1_ | VKRRGDHGRPIITNNPGNREKIGKDMIDRKDQLEMRYFGRTFDDNIHIQLIYNIMDIEKILSIHVNNAL |
| UMGS1840_ | YALNNVLNRGSGDFNDTIGMMLAKPYDVFRNSEKYANFNENIKKPQLSYFGGAFFETGFNTKAQKR |
| genomic | KTEKKSEKDIYYIISLLGFIRQAAVHGYDWNKTDEAAVALYTLDESFDKLYKNTKFRQEARRALDNL |
| SEQ ID NO: | YDTRIDALNSQFLENASKDLTIIFKIYSVTDRSSKIKLIRQYYDFVVKKEYKYLGFSIKQLREMIADDN |
| 5152 | SIIKSKNYDTMRARLNRLIDFVIYINYIENPEKAKKLVENLRSHIGDDEKSRIYASEAKQLWQLLKGPV |
| | MDGILPQMNGKTISSMRADTVISETSVERMKNKTWEPIGKSAHYFIKLVYLLTLFLDGKEINDLVTTL |
| | INKMDNISSFNKLLENIDSKPPYEEEYVIFADSGIMAGELRGLNSFARMEKPDASAKRIMFVEAAQML |
| | GDKGTEQELTKYFDDLMDRNSSKEQKGFRNFIRNNVIESSRFKYLVRYCSVEDVIKFSKNKALIKFVL |
| | KEIPENQILRYYNSCTGNECREFSSEMTLKLAELIENMSFEQFENVRQNARRNSSEETDKLQKQNIIRL |
| | YLTVCYIFFKNLIYVNSRYFLAFYCLERDLRLWDIFENEQSYLMLTEKFMELGKVRKTAKKSKVKNS |
| | GEPSYEDAKHIPYNYIAANLRNADNVAIRKFRNITDHIITVQEAGLYLQDIKNPESYYQIYHYIAQRNL |
| | CDKLKNSNITEKTKEYFALVKNHGSYCKDFVKALCVPFGYNLPRFKNLSIDGLFDMNDKRENRDTT |
| | TEG |
| OPOQ01.1 | MEGINMGRKVHFAKAAGLKSAFAIGNDKVIVTSFGKGNDAILEKTIENDKVINIEQNIDIELMKRDFN |
| SEQ ID NO: | VKRRGDHGRPIITNNPGNREKIGKDMIDRKDQLEMRYFGRTFDDNIHIQLIYNIMDIEKILSIHVNNAL |
| 5153 | YALNNVLNRGSGDFNDTIGMMLAKPYDVFRNSEKYANFNENIKKPQLSYFGGAFFETGFNTKAQKR |
| | KTEKKSEKDIYYIISLLGFIRQAAVHGYDWNKTDEAAVALYTLDESFDKLYKNTKFRQEARRALDNL |
| | YDTRIDALNSQFLENASKDLTIIFKIYSVTDRSSKIKLIRQYYDFVVKKEYKYLGFSIKQLREMIADDN |
| | SIIKSKNYDTMRARLNRLIDFVIYINYIENPEKAKKLVENLRSHIGDDEKSRIYASEAKQLWQLLKGPV |
| | MDGILPQMNGKTISSMRADTVISETSVERMKNKTWEPIGKSAHYFIKLVYLLTLFLDGKEINDLVTTL |
| | INKMDNISSFNKLLENIDSKPPYEEEYVIFADSGIMAGELRGLNSFARMEKPDASAKRIMFVEAAQML |
| | GDKGTEQELTKYFDDLMDRNSSKEQKGFRNFIRNNVIESSRFKYLVRYCSVEDVIKFSKNKALIKFVL |
| | KEIPENQILRYYNSCTGNECREFSSEMTLKLAELIENMSFEQFENVRQNARRNSSEETDKLQKQNIIRL |
| | YLTVCYIFFKNLIYVNSRYFLAFYCLERDLRLWDIFENEQSYLMLTEKFMELGKVRKTAKKSKVKNS |
| | GEPSYEDAKHIPYNYIAANLRNADNVAIRKFRNITDHIITVQEAGLYLQDIKNPESYYQIYHYIAQRNL |
| | CDKLKNSNITEKTKEYFALVKNHGSYCKDFVKALCVPFGYNLPRFKNLSIDGLFDMNDKRENRDTT |
| | TEG |
| IMG_ | MENKENIGKVENQNQKKRSGAKASGLKSTFALGENRVLMTSFGKGNEAIPEKLIVDGAVTDYEKNL |
| 3300029305 | EVTPLKKDFKVKGKHFNNPIVASDPYRRTKVGKDVIDRKEVLEQKYYGRTFDDNIHIQLIYNIMDVE |
| SEQ ID NO: | KILSVHINNILYGLNNVLNRNSDDASDIIGMMRAKPYNDFCVANDKYEQFKGNLENSQLSYYGTAF |
| 5154 | YKTGFDIKAKKERVLKRDEKDIYYILSLLSTVRQFLAHKSDDNRNSKEDNYQVALYTFDEEFDDLYK |
| | EKNIVFRKDARKVLDGLYDSRVISLNESFLKNAKKDLTILFKAYGIENRGDKMKIIREYYDFLIRKSY |
| | KNLGFSLKLLRECIISENKWIADKKYDTMRSRLNRLFDFVTYKYYEDNQTRATVLVEKLRAHSVQK |
| | EKDRIYADEALVLWNGVKSIICNKIKEELNGKNLATMRIDPAIGETSIERLTVLAWKPIGTNATYFTKI |
| | VYLLTLFLDGKEINDMVTTLINKFENIASFNEVLSSTECKTQINREYSIFENSNEVSKELRVLNSFARM |
| | EMPDASTNREMFVEAAKILGYDADRKNLEKYFDTLLDKNASKAEKGFRNFIRNNVINSLRFKYLIKY |
| | CNVEDVKCFSKNKFLVEFVKKNIPEAQILRY |
| IMG_ | MEKMEKIRKSENRKSRAKAAGLKSTFVVADKLALTSFGKGNKANLEKIIKNEEVEEINKPPKFDAKV |
| 3300009671 | EEKQIHIIGKTIKDGITEKPAGMRNDLIGAKQELEKMFFGKTFDDNIHIQLIYNILDIKKIFSVYANNIV |
| SEQ ID NO: | YAVDHLDRTAKDKDVDYLGTLFTGNTYQHLLSVNPGDSKYKLKKDALKRFNTYYESAKSHFIYFG |
| 5155 | DVFYRKPTYEEASAMKEKGQGKKAQKPLVKTEAEVHQILRFLGTIRQCLVHKTDAKRDIIFNPDSMS |
| | NEFKTLIDSYYNGRVKALNDDFIKHNGSSSLPILFHYYGATEENRRAELTEGFYDFIIKRENKNMGFS |
| | LKKIREKMLEKQEASFIKEDGYNSVRHKLYILMDFIIYKTYAADIELQESIVATLRSNLTDEDKEATYE |
| | IQAKALWNKIGDDIVKKLMPLIDGNKIKGYKDEKIEIKKEWLKDVMISTENASYMTKMVYFMTLFL |
| | DGKEINEFLTVLINKFENIQGFFDVLGHNALAEKIEEPKEISKRPLKGFAELAAFTQKSTDVGLTEHLR |
| | LFENSVNISDEIRVTKNFARMERTIPDINRAQYADAAKVLGVIGAGEQD |
| IMG_ | MEKMEKIRKSENRKSRAKAAGLKSTFVVADKLALTSFGKGNKANLEKIIKNEEVEEINKPPKFDAKV |
| 3300026290 | EEKQIHIIGKTIKDGITEKPAGMRNDLIGAKQELEKMFFGKTFDDNIHIQLIYNILDIKKIFSVYANNIV |
| SEQ ID NO: | YAVDHLDRTAKDKDVDYLGTLFTGNTYQHLLSVNPGDSKYKLKKDALKRFNTYYESAKSHFIYFG |
| 5156 | DVFYRKPTYEEASAMKEKGQGKKAQKPLVKTEAEVHQILRFLGTIRQCLVHKTDAKRDIIFNPDSMS |
| | NEFKTLIDSYYNGRVKALNDDFIKHNGSSSLPILFHYYGATEENRRAELTEGFYDFIIKRENKNMGFS |
| | LKKIREKMLEKQEASFIKEDGYNSVRHKLYILMDFIIYKTYAADIELQESIVATLRSNLTDEDKEATYE |
| | IQAKALWNKIGDDIVKKLMPLIDGNKIKGYKDEKIEIKKEWLKDVMISTENASYMTKMVYFMTLFL |
| | DGKEINEFLTVLINKFENIQGFFD |
| UOPK01.1 | MEKMEKIRKSENRKSRAKAAGLKSTFVVADKLALTSFGKGNKANLEKIIKNEEVEEINKPPKFDAKV |
| SEQ ID NO: | EEKQIHIIGKTIKDGITEKPAGMRNDLIGAKQELEKMFFGKTFDDNIHIQLIYNILDIKKIFSVYANNIV |
| 5157 | YAVDHLDRTAKDKDVDYLGTLFTGNTYQHLLSVNPGDSKYKLKKDALKRFNTYYESAKSHFIYFG |
| | DVFYRKPTYKEASAMKEKGQGKKAQKPLVKTKAEVHQILRFLGTIRQCLVHKMDAKWDIIFNPDS |
| | MSNEFKTLIDSYYNGRVKALNDDFIKHNRSSSLPILFHYYGATEENRRAELTEGFYDFIIKRENKNMG |
| | FSLKKIREKMLEKQEASFIKEDGYNSVRHKLYILMDFIIYKPYAADIELQESIVATLRSNLTDEDKEAT |
| | YEIQAKALWNKIGDDIVKKLMPLIDGNKIKGYKDEKIEIKKEWLKDVMISTENASYMTKMVYFMTL |
| | FLDGKEINEFLTVLINKFENIQGFFDVLGHNAFTQKSTDVGLTEHLRLFE |
| IMG_ | MSEEVKKKSKQKSKTKALGLKSILVKEDEIILTSFGKGSKAIKEKVIKQNVITNIASPATFDVKLDEYN |
| 3300009826 | LEVSGKNFITAKPTLPIKVSLSKRQQGKKEFKRRLEEQNLKVQAKREIGEDQIFAKSKLEKHFFGQNF |
| SEQ ID NO: | NDNIKVQIIYNILDIGKILAPYVNDIIYSVNNLWRNCKGDYISGIKFETSYQDCDKKFDKRYKNLKKN |
| 5158 | AIYYSDVFYEVDNNKLVFKNKSDVYNILRVLSLVRQTVMHGYYDNKFLFNDKDLDKELKDLLDWF |
| | YREKVKETNINFLKFNQEANFQILDQLYSNINDKKAIYKLFYDYLIRQEGKNLCFSLKKLREELLKRP |
| | EFSVITSHDFDSIRPKLYHFIDFIILEYYQKQEKLIFEFVEELRANLDEDKKELIYRNKAENIAPSLKVLII |
| | DVLLPLMNGDKIRSFNKKTKEIDSNLFNEIKITDQASYFTKILYFLCLFLDGKEINEMLSTIINKLENIAS |
| | FLEVEGIEKSLDEKLALLSKHFGQKRTNSNKQIIKTNFKENYQMFLDSSQIAEELKIVKAITRMKKKIE |
| | PKEIMYLEAAYLLGYQSSGNQDEDEKNILKQMKLADRGKGVSKDVRNFITNNVIKSQRFLYLVRHM |
| | NPKNIKNVGNNQLIIKFVIDGLPDKQIIRYYNSVTDKNENIPLEMMKQKLVEKITGLKFDDFLLVKNN |
| | PKTKKDLEEKEIKKALVGLYLTILYLVYKNLVNVNARYTIAFYTHERDTKFHGFDMKKPESQKEIVK |
| | LFMEKPYLKTNQIKYLEINLPQLTDEVIFREYRNQIAHLNAVSELDKYLDKLKPINSYFALYHYINQA |
| | SLLERLNYKNNKDKDLNGIKNLIH |
| UPCU01.1 | MLKKRIYQNEADQLWTSYQELFKRIRGFKGAQVKEYSSKNMPIPIQKQIQNILKPAEQVTYFTKLMY |
| SEQ ID NO: | LLTMFLDGKEINDLLTTLINKFDNISSLLKTMEQLELQTTFKEDYTFFQQSSRLCKEITQLKSFARMGN |
| 5159 | PISNLKEVMMVDAIQILGTEKSEQELQSMACFFFRDKNGKKLNTGEHGMRNFIGNNVISNTRFQYLIR |
| | YGNPQKLHTLSQNETVVRFVLSRIAKNQRVQGMNGKNQIDRYYETCGGTNSWSVSEEEKINFLCKIL |
| | TNMSYDQFQDVKQSGAEITAEEKRKKERYKAIISLYLTVLYQLIKNLVNINARYIIAFHCLERDAILYS |
| | SKFNTSINLKKRYTALTEMILGYETDEKARRKDTRTVYEKAEAAKNRHLKNVKWNCKTRENLENA |
| | DKNAIVAFRNIVAHLWIIRDADRFITGMGAMKRYFDCYHYLLQRELGYILEKSNQGSEYTKKSLEKV |
| | QQYHSYCKDFLHMLCLPFAYCIPRYKNLSIAELFDRHEPEAEPKEEASSVNNSQFITT |
| mgm4547164.3_ | VPKFPRILPEAKDISLFSKLIYALTMFLDGKEINDLLTTLINKFENIQCFLKIMPQIGVNANLVDEYIFFN |
| 2 | NSEKIASELKLIKSFAKMGEPVANTKRAMMIDAIKIIGTELNDDELKTLADLFFFDENGNKKGRNQH |
| SEQ ID NO: | GMRNFIINNVISNKRFHYLIRYGDPSHLHEIVKNEAVVKFVLTRIAKLQKKQGQQGKNQIDRYYECCI |
| 5160 | GKGDKKTVKEKIEALTDIITNMNYDQFEKNRDVIERKGNNNAEKEKYKKILSLYLTVIYHILKNIVNI |
| | NSRYVIGFRCVERDAQLYKEKGYSINIGKLDRSGYTSVTKLCLGIANDDPCTRKKAEKEMAQAAQD |
| | TLNRLSEKNSKLFKKYNSYSDTEKEKEFQKQIIREKAATALNDHLRNPSYNHMLREYLSQTDKTACK |
| | IFRNKAAHLEVARYAHQYINEITEVKSYFQLYHYIMQRIIMDNMYDETSGKTKAKENVNDYFDDVR |
| | KNKDYNNQLLKLLCVPFGYCIPRFKNLANEALFDMNEDTEPTPNINPVQTS |
| UWSB01.1 | MSKAKTKTKAVGLKSAFIIDDKVLITSFGKNNNAIPEKEIIGENVKNIEENFSLAVDSDKGAKFKIENN |
| SEQ ID NO: | KHTIETECNNPQYAVQSDLLHAKDKIEMYIFGKTFPNDNIHIQIAYNILDIKKILSLYANNIIFSLNNLR |
| 5161 | HKEEGKFEQDFIGMLYTVNTYAELQKNCSKCICQKICNYKLCKNDGDICKSYKKFKNYLAEINPYLS |
| | YFSEAFYEYIPEKNSKENDKAKRRSEKDIYNIIRTMSLVRQSCFHDLKSTRSAVFNIEDTEIKELLDRL |
| | YQKKIEDVNNGFIKNNGKNIAILADIYNKNTNDEKKKLAADFYNYIILKENKNFGFNLKTVREKLIKK |
| | NFENKICDSVRHKAYILMDYMLYRYYSENEEIKKSFVEDLRSNYDDKNSNYNDKKRNIYEKYADEV |
| | EPKVKGKIELLINKISPQKIRKEKRMDINKEWIKDVQLKNSGNCFPKVIWLMTLFLDGKEINELVSAM |
| | INKFENIQSFIEILIHEHLDHSFDNNGYKMFENSGAIANELKAVKSFSRMQGEIANPTILALHADAARIL |
| | GLNADYTDTELKEHIKTLYHTEENKNQKPKDNNMRNFIVNNVIKSNRFLYLVRYNNPKRSRKLASN |
| | GELVKFVLEGISKDSSGMSKIINRYYVSVNVGIAGEPTNIKEAEKIPLKRKIDKLADMITDMNFDRFK |
| | DVKQKMYIPKNSSQEAKEEAQKKAKLENQKKERDKAVIGLYLTVLYLITKNLVKINARYTIAISCLE |
| | RDTQFFGIDIRDKSETDKRPRKPYITLANKFVNEKYINDKGGHIKKSMQYVANDDRFYREYRNMIAH |
| | LEA |
| OZGD01.1 | MKKKNIRATREALKAQKIKKSQENEALKKQKLAEEAAQKRREELEKKNLAQWEETSAEGRRSRVK |
| SEQ ID NO: | AVGVKSVFVVGDDLYLATFGNGNETVLEKKITPDGKITTFPEEETFTAKLKFAKTELTEATSIGISNG |
| 5162 | RIVLPEISVDNPLHTTMQKNTIKKSAGEDMLQLKDVLENRYFDRSFNNDLHIRLIYNILDIEKILAEYT |
| | TNAVFAIDNVSGCSDDFLSNFSTRNQWDEFQNPEQHREHFGNKDNVICSVKKQQDLFFNFFKNNRIG |
| | YFGKAFFHAESERKIVKKTAKEVYHILTLIGSLRQWITHSTEGGISRLWLYQLEDALSSEYQETMNNC |
| | YNSTIYGLQKDFEKTNAPNLNFLAEILGKNASELAEPYFRFIITKEYKNLGFSIKTLREMLLDQPDLQE |
| | IRENHNVYDSIRSKLYKMMDFVLVYAYSNERKSKADALASNLRSAITEDAKKRVYQNEADQLWTS |
| | YQELFKRIRGFKGAQVKEYSSQNMPIPIQKQIQNILKPAEQVTCFTKLMYLLTMFLDGKEINDLLTTLI |
| | NKFDNISSLLKTMEQLELQTTFKEDYTFFQQSSCLCEEITQLKSFARMGNPISNLKEVMMVDAIQILG |
| | TEKSEQELQSMACFFFRDKNGKKLNAGEHGMRNFIGNNVISNTRFQYLRKH |
| ORJW01.1 | MKKKNIRATREALKAQKIKKSQENEALKKQKLAEEAAQKRREELEKKNLAQWEETSAEGRRSRVK |
| SEQ ID NO: | AVGVKSVFVVGDDLYLATFGNGNETVLEKKITPDGKITTFPEEETFTAKLKFAKTELTEATSIGISNG |
| 5163 | RIVLPEISVDNPLHTTMQKNTIKKSAGEDMLQLKDVLENRYFDRSFNNDLHIRLIYNILDIEKILAEYT |
| | TNAVFAIDNVSGCSDDFLSNFSTRNQWDEFQNPEQHREHFGNKDNVICSVKKQQDLFFNFFKNNRIG |
| | YFGKAFFHAESERKIVKKTAKEVYHILTLIGSLRQWITHSTEGGISRLWLYQLEDALSSEYQETMNNC |
| | YNSTIYGLQKDFEKTNAPNLNFLAEILGKNASELAEPYFRFIITKEYKNLGFSIKTLREMLLDQPDLQE |
| | IRENHNVYDSIRSKLYKMMDFVLVYAYSNERKSKADALASNLRSAITEDAKKRVYQNEADQLWTS |
| | YQELFKRIRGFKGAQVKEYSSQNMPIPIQKQIQNILKPAEQVTCFTKLMYLLTMFLDGKEINDLLTTLI |
| | NKFDNISSLLKTMEQLELQTTFKEDYTFFQQSSCLCEEITQLKSFARMGNPISNLKEVMMVDAIQILG |
| | TEKSEQELQSMACFFFRDKNGKKLNAGEHGMRNFIGNNVISNTRFQYLRKH |
| OZBA01.1 | MKKKNIRATREALKAQKIKKSQENEALKKQKLAEEAAQKRREELEKKNLAQWEETSAEGRRSRVK |
| SEQ ID NO: | AVGVKSVFVVGDDLYLATFGNGNETVLEKKITPDGKITTFPEEETFTAKLKFAKTELTEATSIGISNG |
| 5164 | RIVLPEISVDNPLHTTMQKNTIKKSAGEDMLQLKDVLENRYFDRSFNNDLHIRLIYNILDIEKILAEYT |
| | TNAVFAIDNVSGCSDDFLSNFSTRNQWDEFQNPEQHREHFGNKDNVICSVKKQQDLFFNFFKNNRIG |
| | YFGKAFFHAESERKIVKKTAKEVYHILTLIGSLRQWITHSTEGGISRLWLYQLEDALSSEYQETMNNC |
| | YNSTIYGLQKDFEKTNAPNLNFLAEILGKNASELAEPYFRFIITKEYKNLGFSIKTLREMLLDQPDLQE |
| | IRENHNVYDSIRSKLYKMMDFVLVYAYSNERKSKADALASNLRSAITEDAKKRVYQNEADQLWTS |
| | YQELFKRIRGFKGAQVKEYSSQNMPIPIQKQIQNILKPAEQVTCFTKLMYLLTMFLDGKEINDLLTTLI |
| | NKFDNISSLLKTMEQLELQTTFKEDYTFFQQSSCLCEEITQLKSFARMGNPISNLKEVMMVDAIQILG |
| | TEKSEQELQSMACFFFRDKNGKKLNAGEHGMRNFIGNNVISNTRFQYLRKH |
| OZZW01.1 | MKKKNIRATREALKAQKIKKSQENEALKKQKLAEEAAQKRREELEKKNLAQWEETSAEGRRSRVK |
| SEQ ID NO: | AVGVKSVFVVGDDLYLATFGNGNETVLEKKITPDGKITTFPEEETFTAKLKFAKTELTEATSIGISNG |
| 5165 | RIVLPEISVDNPLHTTMQKNTIKKSAGEDMLQLKDVLENRYFDRSFNNDLHIRLIYNILDIEKILAEYT |
| | TNAVFAIDNVSGCSDDFLSNFSTRNQWDEFQNPEQHREHFGNKDNVICSVKKQQDLFFNFFKNNRIG |
| | YFGKAFFHAESERKIVKKTAKEVYHILTLIGSLRQWITHSTEGGISRLWLYQLEDALSSEYQETMNNC |
| | YNSTIYGLQKDFEKTNAPNLNFLAEILGKNASELAEPYFRFIITKEYKNLGFSIKTLREMLLDQPDLQE |
| | IRENHNVYDSIRSKLYKMMDFVLVYAYSNERKSKADALASNLRSAITEDAKKRVYQNEADQLWTS |
| | YQELFKRIRGFKGAQVKEYSSQNMPIPIQKQIQNILKPAEQVTCFTKLMYLLTMFLDGKEINDLLTTLI |
| | NKFDNISSLLKTMEQLELQTTFKEDYTFFQQSSCLCEEITQLKSFARMGNPISNLKEVMMVDAIQILG |
| | TEKSEQELQSMACFFFRDKNGKKLNAGEHGMRNFIGNNVISNTRFQYLRKH |
| UAFA01.1 | MKKKNIRATREALKAQKIKKSQENEALKKQKLAEEAAQKRREELEKKNLAQWEETSAEGRRSRVK |
| SEQ ID NO: | AVGVKSVFVVGDDLYLATFGNGNETVLEKKITPDGKITTFPEEETFTAKLKFAKTELTEATSIGISNG |
| 5166 | RIVLPEISVDNPLHTTMQKNTIKKSAGEDMLQLKDVLENRYFDRSFNNDLHIRLIYNILDIEKILAEYT |
| | TNAVFAIDNVSGCSDDFLSNFSTRNQWDEFQNPEQHREHFGNKDNVICSVKKQQDLFFNFFKNNRIG |
| | YFGKAFFHAESERKIVKKTAKEVYHILTLIGSLRQWITHSTEGGISRLWLYQLEDALSSEYQETMNNC |
| | YNSTIYGLQKDFEKTNAPNLNFLAEILGKNASELAEPYFRFIITKEYKNLGFSIKTLREMLLDQPDLQE |
| | IRENHNVYDSIRSKLYKMMDFVLVYAYSNERKSKADALASNLRSAITEDAKKRVYQNEADQLWTS |
| | YQELFKRIRGFKGAQVKEYSSQNMPIPIQKQIQNILKPAEQVTCFTKLMYLLTMFLDGKEINDLLTTLI |
| | NKFDNISSLLKTMEQLELQTTFKEDYTFFQQSSCLCEEITQLKSFARMGNPISNLKEVMMVDAIQILG |
| | TEKSEQELQSMACFFFRDKNGKKLNAGEHGMRNFIGNNVISNTRFQYLRKH |
| OZDP01.1 | MKKKNIRATREALKAQKIKKSQENEALKKQKLAEEAAQKRREELEKKNLAQWEETSAEGRRSRVK |
| SEQ ID NO: | AVGVKSVFVVGDDLYLATFGNGNETVLEKKITPDGKITTFPEEETFTAKLKFAKTELTEATSIGISNG |
| 5167 | RIVLPEISVDNPLHTTMQKNTIKKSAGEDMLQLKDVLENRYFDRSFNNDLHIRLIYNILDIEKILAEYT |
| | TNAVFAIDNVSGCSDDFLSNFSTRNQWDEFQNPEQHREHFGNKDNVICSVKKQQDLFFNFFKNNRIG |
| | YFGKAFFHAESERKIVKKTAKEVYHILTLIGSLRQWITHSTEGGISRLWLYQLEDALSSEYQETMNNC |
| | YNSTIYGLQKDFEKTNAPNLNFLAEILGENASELAEPYFRFIITKEYKNLGFSIKTLREMLLDQPDLQEI |
| | RENHNVYDSIRSKLYKMMDFVLVYAYSNERKSKADALASNLRSAITEDAKKRVYQNEADQLWTSY |
| | QELFKRIRGFKGAQVKEYSSQNMPIPIQKQIQNILKPAEQVTCFTKLMYLLTMFLDGKEINDLLTTLIN |
| | KFDNISSLLKTMEQLELQTTFKEDYTFFQQSSCLCEEITQLKSFARMGNPISNLKEVMMVDAIQILGTE |
| | KSEQELQSMACFFFRDKNGKKLNAGEHGMRNFIGNNVISCL |
| UPCU01.1_2 | MKKKNIRATREALKAQKIKKSQENEALKKQKLAEEAAQKRREELEKKNLAQWEETSAEGRRSRVK |
| SEQ ID NO: | AVGVKSVFVVGDDLYLATFGNGNETVLEKKITPDGKITTFPEEETFTAKLKFAQTEPTVATSIGISNG |
| 5168 | RIVLPEISVDNPLHTTMQKNTIKRSAGEDILQLKDVLENRYFDRSFNDDLHIRLIYNILDIEKILAEYTT |
| | NAVFAIDNVSGCSDDFLSNFSTRNQWDEFQNPEQHREHFGNKDNVICSVKKQQDLFFNFFKNNRIGY |
| | FGKAFFHAESERKIVKKTEKEVYHILTLIGSLRQWITHSTEGGISRLWLYQLEDALSREYQETMNNCY |
| | NSTIYGLQKDFEKTNAPNLNFLAEILGKNASELAEPYFRFIITKEYKNLGFSIKTLREMLLDQPDLQEIR |
| | ENHNVYDSIRSKLYKMIDFVLVYAYSNERKSKADALASNLRSAITEDAKKKDLSERSGSVVDKLSGII |
| | QENSRFQRCSSKRILKQKHANPHSEADSKYLKASGTSDLLHKVDVFVDDVLRW |
| OGRM01.1 | MVTAERKMTMKKREECLGSREELEQKNLKKWEETNAENRRSRAKAVGVKSVFVVGEDLYLATFG |
| SEQ ID NO: | NGNETLLEKKITPDGTITSFPKEEAFTAKLKFAQTESTEATSIGISNGRIVLPEVPVDNPCYAAPQAKT |
| 5169 | AKKVAGEDLLQLKEVLEKRYFGCSFDDDLHIRLIYNILDIEKILAEYVTNAVFSIDNVSGNAHDFLGY |
| | LSTRNSYDAFMHPEKYPEHFENKSDLIERVRKQGDDFLAFVDNKRIGYFGKAFFYQDGRKEIEKPDG |
| | EIYHLLTLIGSLRQWITHSDEREEGTSRTWLYQLEKFLLPEYQETMNVNYNDIVKELTTNFTKTNATN |
| | LNFLAELLHVPVKAIAESYFRFAITKEYKNLGFCIKTIREILLERRELSDIKENHAVYDSIRSKLYKMM |
| | DFVLVHAYESEEGKKEAEELASSLRFALTEEEKESIYLNEAERLWKMYGDKLLKIKDFKGSQVNLYS |
| | YKSKPVDVQLPAILKPAKEVTCFTKLMYILTMFLDGKEINDLLTTLINKFDNINSLLKTMEQLELQTA |
| | FVKEYTFFSQSQRLCAEITQLKSFARMGKPVSNAKEAMMIDAIQILGTDKTEKELETMAKRFFRDGN |
| | GKLLK |
| mgm4547164.3 | MSKHRTSYAKAMGLKSILVSDSVTEMLSFAKGSDARLEKMVEDDRITDLIDKNEEAFSAEFVKNRN |
| SEQ ID NO: | DNRYGYSIKNSKFSHPENRSVIAADPLLKGSVKSDMLELKSTLEKRIFGTESNGNALIQIAYNIQDIEKI |
| 5170 | LAEYITNIVYAVNNIAGMDKDIIGFGKFSPEHTYEEFSEPDNHKEHYNNDKYLINAVKNQYEEFDAFL |
| | DNPRFGYFGKAFFQKKQNRSTDFVIKYDIECYHILSLISGLRNWIVHNNEDKARVSRTWLYDLENNL |
| | DHEYIETLDYMYDDIANEIVNSFSTRNSANVNYISEILNISSDKLAEQYFRFSIMKDQKNLGFNIIKLRE |
| | TMLDRQEMSDIHENHNVFDTIRSKLYTMMDFVIYRYYMEEDKKTTAENNNLPDERKKLSKKDIFVI |
| | NLRGCFNDEHKDKLYAEEAEQLWKVLGNIMKQXKACA |
| IMG_ | MKGTVVKNGKFLEIKGENGKIYQLKPFHLPEGKTIQWLNAGDRVTFRTKDKGRIAEKSVDKIKTYK |
| 3300018493 | LPVTPRTHEEIFGDKSAQRPKTKAKAIGLKSLLYYDDKIIATSFGKGNKANVEKIVSDKIENIYDPANY |
| SEQ ID NO: | SIFINDKLKSFVLQDKKGENSVSINLFNATISNEADHKGEITINAGQVGRDQIGLKDALEEKIFGKTYN |
| 5171 | DNIHVQIAYNILDVEKILTEYINDIVYAMNNAADISPDRDLVGYLGHLFNYQKYWSNPKSANNKPNF |
| | DKFVNSPRLINYGEAFYTVGRDGPRRKSEREIFYMLSFVGYARQFIAHEYGKHISIYNPAKAADKEV |
| | ADFLDRLFADKIDEVNSDFLKLNAKNIAIICDIFPNENRNTLIKEFYQFSVLKAHKNMGFSIKTLRETL |
| | LALEQAQQLRSYIMQHQDFRRQVNTQLDFMIYKRYKDSSRAKSFADALRATARKEDKPALYIAEAS |
| | KLYAETAKFITTGILAALDSKQAQKLENFPAIPPEAIKDVCIGSQAYDFVKLIYLFTLLLDGKEINNLLT |
| | GLISKFDNINSLGRIYTEITGLVNLEDRYVKFEKDYSIFKDCGRVVAQLQMLNSFARMQKRPDSQTN |
| | AKAKKAKDPFAKTDFGDAVMVLGVSPQDKKAAIDKLTPGMKNYISNNVLNSSRFRYLVRYADVKN |
| | VSILAKSKVLTEFVLADIPDNQIERYTESCGGNSSLSVKEKRQFLAQKIAELSIDNMYVEMDKKATEQ |
| | QMLQRENNKNILGLYLTVLYLVCKNLVNVNARYLMAFHCLARDSYLLGKPYKKDNVYDYAVLTA |
| | DYIRHSKNAHMAQFLATDTEKSNNRLIHQ |
| IMG_ | LSTKKRFRYSVAAKAAGLKSSLAVDTDRTVMTSFGHGNAAILEKEIVDGEISVLNIENPAFDAVINDK |
| 3300024303 | KYALTGHHAGVHALVDQPQNRSDAVHIRGALEKKYFGDTFADNIHVQIAYNILDITKILTVYANNV |
| SEQ ID NO: | VYALNNLVHADDDTQADELDSLGNFSAGTSYAKSKSKSKSKQQDFVELFIKKKEIHGYFGDTFAFL |
| 5172 | DKRIADADKEKQVYAMLACLGSLRQACSHYRIRYSVNGKNVDADADTWLFSSAQLDQTDPLFSEM |
| | LNRIYSHKIKTVNQNFFENNRKANFPILKKMYPETTLKVLMNEYYDFSIRKGYKNFGFSIKSLREALL |
| | SPQYESLIGVQIKDNKEYDTVRSKLYQLFDFALTRYFNQHPDMVDAFVVELRSLAKDEDAKNAVYE |
| | KYAKAVWNDVKQPIAVMLSYMNGSAIKNIKAFELKPDQKELNGIMNSNALDVPHFCKLVYFLTRFL |
| | DGKEINDLLTTLVNKFDNIHSFNQVLTALGLSASYEADYKIFEDSGRVVEYLREINSFARMTVDMEKI |
| | KRSAYKKALLILGSSKYSDEDLDARVDEMLGVDYNQNGEKIKVRVDTGFRNFIANNVVESSRFHYLI |
| | RYCHPRKIRNLAGNAALIEYQLRRLPELQILRYYEACTEPIKRTARTMDEKIGTLIDLIVKMDFSQFED |
| | VQQNDRVRVFSDAEKKEKIRKMREKQRYQSIISLYLTMLYLNVKNLVNINARYVMAFQAWERDNY |
| | LLLQLSGKEAEAEYLNLTRHFIEPLDGA |
| IMG_ | MKSKVIKSPAKAAGLKSILVHDNIMYLTYFDKGPQGGLEKKVTKQKDVYEVLDIANPIHFDAKANR |
| 3300028603 | VNIDIQGTNGIIAKPSNPFYSNKVIGMDLVRTKYALEQQIFGQRFMNDNIHVQIAYQVLDILKALVPYI |
| SEQ ID NO: | NDITYSLNAKIALKDLNLDKDDFFEINPKKIDDSKNERLLQGMMDAIKNNAVYFQEVFYDIKKDKLI |
| 5173 | LKSTDLIKSYLKILIKLRQTVSHYDNSNSHYIFSGGENKEDIQLLKNLFENKRDKVNKDFIDLNKRSNF |
| | FILDKIFNIHNLQQSNLMHQYFYEYSILSMHKNIGFSIKTLREEILKLEGASRLLSTEYDSVRGKMYNL |
| | LDFILYHYFEIETRGVITQSIVEDLRKNQTEEGKLDIYSTLAVKTLSAVKSKLETLEGLMNGDTIQKIN |
| | REKTVTRDFQPKLVKGNFLVFIIYFITLFLDKKEVNDLTTTIINKLENIAAFQKVYKEITGHNIEFLNEY |
| | QVLENAEQTAKEMRFVQVMGLKKPELIITHSLLLDAHKILGYQPRGDEKDILKETNATKEFRNFLINS |
| | VIKSRKFVYLIKHVRAEDISKMAKNKYLVEYVLHRIAKNAPSQIDRYYKTITGQSDGTSKEKIEKLTH |
| | RISQLKYSDFAAIKTKTVNPFREQSKALIGLYLTVLQILYKNLVNVNARYTMGFWALEKDTLLHQIP |
| | YKGHIPLLGLVEKFLYNSDDPEEAWLKKRQIQIIKQNLSDLGQGKEKHILIKYRNLIAHQNVIRKSHE |
| | LLKDIRHVDSYFQLFHIAIQKQLNEMIKDGTVNPSSKIKDQLAWVEKNNQVSKNLLHTLNLPFAYNT |
| | ARYNKLSIGDLFDRNEEK |
| IMG_ | MKSKVIKSPAKAAGLKSILVHDNIMYLTYFDKGPQGGLEKKVTKQKDVYEVLDIANPIHFDAKANR |
| 3300027711 | VNIDIQGTNGIIAKPSNPFYSNKVIGMDLVRTKYALEQQIFGQRFMNDNIHVQIAYQVLDILKALVPYI |
| SEQ ID NO: | NDITYSLNAKIALKDLNLDKDDFFEINPKKIDDSKNERLLQGMMDAIKNNAVYFQEVFYDIKKDKLI |
| 5174 | LKSTDLIKSYLKILTKLRQTVSHYDNSNSHYIFSGGENKEDIQLLKNLFENKREKVNKDFIDLNKRSN |
| | FFILDKIFNIHNLQQSNLMHQYFYEYSILSMHKNIGFSIKTLREEILKLEGASRLLSTEYDSVRGKMYN |
| | LLDFILYHYFEIETRGVITQSIVEDLRKNQTEEGKLDIYSRLAVKTLSAVKSKLETLEGLMNGDTIQKI |
| | NREKTVIRDFQPKLVKGNFLVFIIYFITLFLDKKEVNDLTTTIINKLENIAAFQKVYKEITGHNIEFLNE |
| | YQVLENAEQTAKEMRFVQVMGLKKPELIITHSLLLDAHKILGYQPRGDEKDILKETNATKEFRNFLI |
| | NSVIKSRKFVYLIKHVRAEDISKMAKNKYLVEYVLHRIAKNAPSQIDRYYKTITGQSDGTSKEKIEKL |
| | THRISQLKYSDFAAIKTKTVNPFREQSKALIGLYLTVLQILYKNLVNVNARYTMGFWALEKDTLLHQ |
| | IPYKGHIPLLGLVEKFLYNSDDPEEAWLKKSQIQIIKQNLSDLGQGKEKHILIKYRNLIAHQNVIRKSH |
| | ELLKDIRHVDSYFQLFHIAIQKQLNEMIKDGTVNPSSKIKDQLAWVEKNNQVSKNLLHTLNLPFAYN |
| | TARYNKLSIGDLFDRNEEK |
| IMG_ | LFSLYDIQDLSSQKAIDLARKFYNWNVIHGQKNLGVSVRFLRENLITLSEASFLSLKEFDTIRSKLYLF |
| 3300034093 | LDFIIATDYLSLPNQLNPLVEKLRMAKSEEDKDKIYLEESRLIWQTIKDRVHIKLVPLMNIKNIKTLPK |
| SEQ ID NO: | RSLDEKHYRAIMIKNDASSFSKLMYVFSTFLDMKESNDLLSGLINKFSEIASMNNLLNESSSIFDNSRI |
| 5175 | NDHSKLFEKSSVIAKELMIVKALIQKQKEVVFSDRSLLDAALTIGIPEELQNIDYVKGLFKQYEQNNF |
| | KNFLINNVVKSRRFIYLMRYVNPLIIKQMMTHLPSIRFILNRLPIEQIDRYIFSALGKNPSGFNSVNTKI |
| | DELANQLIKIKLEQFTSVVQGRPKKFANQLSKPNFQENNFKEKQKALLGLYLTVPYLFFKGLVNVNS |
| | RYTIALHAFERDNDIILNSPEIFKQEKPDYLMLTKHYLKLGKFKTRVKTYLIDNQAHFNANMFKHYR |
| | DQIAHLGIIKNAVRFFKSNHHVMKSYFEIYHTILGLGFNDHVHHLDHSQAFLATAIDSIRKYGTYSKD |
| | LLNVLHTPFAYNLPRYKNLSIHDLFDKNEITKEK |
| IMG_ | MAKNKIRPGDKRKQDAQAAAAKHRVQNEAREKEIAEEAAKKEAAAAVKKSSITFSDEKGTVKTKS |
| 3300012983 | SAKAAGLKSAFVIGDDVLLTSFGKGNDVILEKRFSKENNATDFDNKLNITPISSRVADDKKTKYEFNI |
| SEQ ID NO: | RNKATTDDPRINGSASPARQDMLGQKAKLEKKYFGVEFNDNIHIQLIHNILDIEKILAIHINNIVTSLN |
| 5176 | NIMDVDEPKTGDIIGYSSLLKTWNEFENKTETKLFTDFENFYNNSKLSYFGSGFYDTGFDTKAQLNS |
| | KKRSKEEVYYILCLLGQLRQEITHGNSNYIYNLANDTSEAATEVKSILNSFYGAQVEN |
| UPLX01.1 | MILILGEGTIRMAKKKNARQRREEEKNRIKAIIEKIKNKVVEKEETEEIVENNETKNVESIVVEPKKKS |
| SEQ ID NO: | LAKASGVKSVFINNDEIIMDTSFGRGNDAVIEKIIKDNNIDNENKDKPVYDVVAIENEGNIIKVQSERFK |
| 5177 | AIESANTEIPPERNGMDLIKRKDKLEEVYFGHTFNDNIHIQLIYNILDIEKILSVYINNIVYALGNLERK |
| | DTDEEKDLIGYSSARAKYEDFIENEKLEDRKKLLEEFIENGDRLGYFGNVFFKNDKELKSKKEIYNIL |
| | GLLGSLRQFCFHYNEAVFENEEGKINQDIAPFSTLSTTYCFLTKSFIYEALRYIRLAL |
| GCA_ | MERQKRKMKSKSKMAGVKSVFVIGDELLMTSFGDGDDAVLEKDIDENGVVNDCRNPAAYDAVYG |
| 900544545.1_ | TDSIRVKKTNNNIRAKVNNPLAKSNIRSEESALFRTRVNEYKREQKDKYETLFFGKTFDDNIHIQLISK |
| UMGS692_ | ILDIEKTFSVVIGNIVYAINNLSLEQSIDRPIDIFGDKNTQGISLREDNDYLKTMLPRCEYLFHNILNSDS |
| genomic | DNNSKMNYNKVNKGKEEKDNRNNENIEKLKKALEVIKIIRVDSFHGVDGIKGDQKFPRSKYNLAVN |
| SEQ ID NO: | YNEEIQKTISEPFNRKVEEVQQDFYRNSCVNIDFLKEIMYGSNYTDRGSDSLECSYFNFAILKQNKNM |
| 5178 | GFSITSIRECLLDLYELNFESMQNLRPRANSFCDFLIYDYYCKNESERANLVDCLRSAASEEEKKNIYF |
| | QTAERVKEKFRNAFNRISRFDASYIKNSREKNLSGGSSLPKYSFIEGFTKRSKKINDNDEKNADLFCN |
| | MLYYLAQFLDGKEINIFLTSIHNIFQNIDSFLKVMKEKGMECKFQKDFKMFSHAGHVAKKIEIVISLA |
| | KMKKTLDFYNAQALKDAVTILGVSKKHQYLDMNSYLDFYMFDNRSGATGKNAGKDHNLRNFLVS |
| | NVIRSRKFNYLSRYSNLAEVKKLAQNPSLVQFVLSRIEPSLICRYYESSQGISSEGITIDEQIKKLTGIIV |
| | DMNIDLFENINNGEIGMRYSKATPQSIERRNQMRVCWFVS |
| OMCP01.1 | MERQKRKMKSKSKMAGVKSVFVIGDELLMTSFGDGDDAVLEKDIDENGVVNDCRNPAAYDAVYG |
| SEQ ID NO: | TDSIRVKKTNNNIRAKVNNPLAKSNIRSEESALFRTRVNEYKREQKDKYETLFFGKTFDDNIHIQLISK |
| 5179 | ILDIEKTFSVVIGNIVYAINNLSLEQSIDRPIDIFGDKNTQGISLREDNDYLKTMLPRCEYLFHNILNSDS |
| | DNNSKMNYNKVNKGKEEKDNRNNENIEKLKKALEVIKIIRVDSFHGVDGIKGDQKFPRSKYNLAVN |
| | YNEEIQKTISEPFNRKVEEVQQDFYRNSCVNIDFLKEIMYGSNYTDRGSDSLECSYFNFAILKQNKNM |
| | GFSITSIRECLLDLYELNFESMQNLRPRANSFCDFLIYDYYCKNESERANLVDCLRSAASEEEKKNIYF |
| | QTAERVKEKFRNAFNRISRFDASYIKNSREKNLSGGSSLPKYSFIEGFTKRSKKINDNDEKNADLFCN |
| | MLYYLAQFLDGKEINIFLTSIHNIFQNIDSFLKVMKEKGMECKFQKDFKMFSHAGHVAKKIEIVISLA |
| | KMKKTLDFYNAQALKDAVTILGVSKKHQYLDMNSYLDFYMFDNRSGATGKNAGKDHNLRNFLVS |
| | NVIRSRKFNYLSRYSNLAEVKKLAQNPSLVQFVLSRIEPSLICRYYESSQGISSEGITIDEQIKKLTGIIV |
| | DMNIDLFENINNGEIGMRYSKATPQSIERRNQMRVCWFVS |
| OMEK01.1 | MERQKRKMKSKSKMAGVKSVFVIGDELLMTSFGDGDDAVLEKDIDENGVVNDCRNPAAYDAVYG |
| SEQ ID NO: | TDSIRVKKTNNNIRAKVNNPLAKSNIRSEESALFRTRVNEYKREQKDKYETLFFGKTFDDNIHIQLISK |
| 5180 | ILDIEKTFSVVIGNIVYAINNLSLEQSIDRPIDIFGDKNTQGISLREDNDYLKTMLPRCEYLFHNILNSDS |
| | DNNSKMNYNKVNKGKEEKDNRNNENIEKLKKALEVIKIIRVDSFHGVDGIKGDQKFPRSKYNLAVN |
| | YNEEIQKTISEPFNRKVEEVQQDFYRNSCVNIDFLKEIMYGSNYTDRGSDSLECSYFNFAILKQNKNM |
| | GFSITSIRECLLDLYELNFESMQNLRPRANSFCDFLIYDYYCKNESERANLVDCLRSAASEEEKKNIYF |
| | QTAERVKEKFRNAFNRISRFDASYIKNSREKNLSGGSSLPKYSFIEGFTKRSKKINDNDEKNADLFCN |
| | MLYYLAQFLDGKEINIFLTSIHNIFQNIDSFLKVMKEKGMECKFQKDFKMFSHAGHVAKKIEIVISLA |
| | KMKKTLDFYNAQALKDAVTILGVSKKHQYLDMNSYLDFYMFDNRSGATGKNAGKDHNLRNFLVS |
| | NVIRSRKFNYLSRYSNLAEVKKLAQNPSLVQFVLSRIEPSLICRYYESSQGISSEGITIDEQIKKLTGIIV |
| | DMNIDLFENINNGEIGMRYSKATPQSIERRNQMRVCWFVS |
| OLYJ01.1 | MERQKRKMKSKSKMAGVKSVFVIGDELLMTSFGDGDDAVLEKDIDENGVVNDCRNPAAYDAVYG |
| SEQ ID NO: | TDSIRVKKTNNNIRAKVNNPLAKSNIRSEESALFRTRVNEYKREQKDKYETLFFGKTFDDNIHIQLISK |
| 5181 | ILDIEKTFSVVIGNIVYAINNLSLEQSIDRPIDIFGDKNTQGISLREDNDYLKTMLPRCEYLFHNILNSDS |
| | DNNSKMNYNKVNKGKEEKDNRNNENIEKLKKALEVIKIIRVDSFHGVDGIKGDQKFPRSKYNLAVN |
| | YNEEIQKTISEPFNRKVEEVQQDFYRNSCVNIDFLKEIMYGSNYTDRGSDSLECSYFNFAILKQNKNM |
| | GFSITSIRECLLDLYELNFESMQNLRPRANSFCDFLIYDYYCKNESERANLVDCLRSAASEEEKKNIYF |
| | QTAERVKEKFRNAFNRISRFDASYIKNSREKNLSGGSSLPKYSFIEGFTKRSKKINDNDEKNADLFCN |
| | MLYYLAQFLDGKEINIFLTSIHNIFQNIDSFLKVMKEKGMECKFQKDFKMFSHAGHVAKKIEIVISLA |
| | KMKKTLDFYNAQALKDAVTILGVSKKHQYLDMNSYLDFYMFDNRSGATGKNAGKDHNLRNFLVS |
| | NVIRSRKFNYLSRYSNLAEVKKLAQNPSLVQFVLSRIEPSLICRYYESSQGISSEGITIDEQIKKLTGIIV |
| | DMNIDLFENINNGEIGMRYSKATPQSIERRNQMRVCWFVS |
| OMCO01.1 | MERQKRKMKSKSKMAGVKSVFVIGDELLMTSFGDGDDAVLEKDIDENGVVNDCRNPAAYDAVYG |
| SEQ ID NO: | TDSIRVKKTNNNIRAKVNNPLAKSNIRSEESALFRTRVNEYKREQKDKYETLFFGKTFDDNIHIQLISK |
| 5182 | ILDIEKTFSVVIGNIVYAINNLSLEQSIDRPIDIFGDKNTQGISLREDNDYLKTMLPRCEYLFHNILNSDS |
| | DNNSKMNYNKVNKGKEEKDNRNNENIEKLKKALEVIKIIRVDSFHGVDGIKGDQKFPRSKYNLAVN |
| | YNEEIQKTISEPFNRKVEEVQQDFYRNSCVNIDFLKEIMYGSNYTDRGSDSLECSYFNFAILKQNKNM |
| | GFSITSIRECLLDLYELNFESMQNLRPRANSFCDFLIYDYYCKNESERANLVDCLRSAASEEEKKNIYF |
| | QTAERVKEKFRNAFNRISRFDASYIKNSREKNLSGGSSLPKYSFIEGFTKRSKKINDNDEKNADLFCN |
| | MLYYLAQFLDGKEINIFLTSIHNIFQNIDSFLKVMKEKGMECKFQKDFKMFSHAGHVAKKIEIVISLA |
| | KMKKTLDFYNAQALKDAVTILGVSKKHQYLDMNSYLDFYMFDNRSGATGKNAGKDHNLRNFLVS |
| | NVIRSRKFNYLSRYSNLAEVKKLAQNPSLVQFVLSRIEPSLICRYYESSQGISSEGITIDEQIKKLTGIIV |
| | DMNIDLFENINNGEIGMRYSKATPQSIERRNQMRVCWFVS |
| OUQC01.1 | MERQKRKMKSKSKMAGVKSVFVIGDELLMTSFGDGDDAVLEKDIDENGVVNDCRNPAAYDAVYG |
| SEQ ID NO: | TDSIRVKKTNNNIRAKVNNPLAKSNIRSEESALFRTRVNEYKREQKDKYETLFFGKTFDDNIHIQLISK |
| 5183 | ILDIEKTFSVVIGNIVYAINNLSLEQSIDRPIDIFGDKNTQGISLREDNDYLKTMLPRCEYLFHNILNSDS |
| | DNNSKMNYNKVNKGKEEKDNRNNENIEKLKKALEVIKIIRVDSFHGVDGIKGDQKFPRSKYNLAVN |
| | YNEEIQKTISEPFNRKVEEVQQDFYRNSCVNIDFLKEIMYGSNYTDRGSDSLECSYFNFAILKQNKNM |
| | GFSITSIRECLLDLYELNFESMQNLRPRANSFCDFLIYDYYCKNESERANLVDCLRSAASEEEKKNIYF |
| | QTAERVKEKFRNAFNRISRFDASYIKNSREKNLSGGSSLPKYSFIECS |
| IMG_ | MKKSKVKLNGVKAVYHISPDVRVTTAFGRGNNSVLDKRIENGTIEELQNHSDIDVNISRKTYSFRKK |
| 3300031760 | SLKKDAGQFSVPDNTNDQLGIRKELEEEIFGEKFDDNIHIQAAYAVNDIIKTLSVAANLAATAINGLD |
| SEQ ID NO: | RENTEYDMIGFYIIPHITYQTYAGNKKPKFDEFIGRVKAQGTFSYFPDILPKFREESEEESDKEKLYYL |
| 5184 | MCIVSLIRNSTIHYAAGNSRNADSMDYIFGELNKEALTETADDLIKSKINSINKGFSNNQKNNIYRLLK |
| | AKADTPENTARLIRRLYAFTIRKQDKNLGFSLKKLRECAIRSIEDMKGLLGDTYDTVRSKLYTLVDF |
| | VVYSYLKYHKEGKKFSEEMVDQLRAAESDKDKNIVYCQGAKRLYNIKIISQTINALISDIKSDFDQPK |
| | HGNECYQPINDGMKEAEKDFITTDQLSLFTKFIYVLCQFLDGKNINILLSSLISKFQQIEAFNGDIRKLN |
| | LNIRDDGKIGYDSKKYSIFEKSGQISMELDMLRGVIKMDINDLNAYKTMIKDALLVIGVNESDIDSIY |
| | EYYFNQKDKKNSVAGFFRNNIINSRRFRYIIKYINPSDAYRIIQNENVRNYVLGRMNDAIIDRYAHSV |
| | GIEDKVHDKRKVLSDILSTVKFDNFTELKYINPKDRNKDQKAKGREKPKAILGLYLTIVYIVVKSLVR |
| | INSQYVMAVYHLERDSRLCGVSSNDNYPLSMTKRYCDRDNHLLKEKHIVKLERYQNTPEKICTTYR |
| | NAIAHLSAVRKGVKYIGDIQKTDSYFGIYHYCMQKLLYSADSADKQPFADFVSSVFGDEKELDKLRS |
| | GIYSQAILRALNYPFGYNPARYKNLSYEKIFMRAWQDEDTNKKT |
| IMG_ | MKKSKVKLNGVKAVYHISPDVRVTAAFGRGNNSVLDKRIENGTIEELQNHSDIDVNISRKTYSFRKK |
| 3300028886 | SLKKDAGQFSVPDNTNDQLGIRTELEEEIFGGNFDDNIHIQAAYAVNDIIKTLSVAANLAETAINGLD |
| SEQ ID NO: | RKNTENDIIGFYIIPQITYQTYAGNKKPKFDEFIGRVKAQGTFSYFPDILPKFKEESEEESDKEKLYYLM |
| 5185 | CIISLIRNSATHSKNSNSDTTDYIFGKLNSDNKEALTETADNLIKSKIDSINEGFSKNQKNNIYRLLKAG |
| | ADTTENTARLIRRLYAFIIKKQDKNLGFSLKKLRECAIRSIEYMKYLPGKKYDTVRSKLYTLVDFVVY |
| | SYLKYHKEGKKFSEEMVDQLRAAESDEDKNIVYCQGAERLYNIEIIRRTIKALIKDMDIKSEFKQPDK |
| | DNTYYQPIMDGMEEAKKDFITTDQLSLFTKFIYVLCQFLDGKNINILLSSLISKFQQIEAFNGDIRKLNL |
| | NIRDDGKIGYDSKKYSIFEKSGQIADDLDRLRGVIKMDINDLNAYETMIKDALRVIGVDESDIESIYQT |
| | H |
| IMG_ | MKKSKVKLNGVKAVYHISPDVRVTAAFGRGNNSVLDKRIENGTIEELQNHSDIDVNISRKTYSFRKK |
| 3300032007 | SLKKDAGQFSVPDNTNDQLGIRTELEEEIFGGNFDDNIHIQAAYAVNDIIKTLSVAANLAETAINGLD |
| SEQ ID NO: | RKNTENDIIGFYIIPQITYQTYAGNKKPKFDEFIGRVKAQGTFSYFPDILPKFKEESEEESDKEKLYYLM |
| 5186 | CIISLIRNSATHSKNSNSDTTDYIFGKLNSDNKEALTETADNLIKSKIDSINEGFSKNQKNNIYRLLKAG |
| | ADTTENTARLIRRLYAFIIKKQDKNLGFSLKKLRECAIRSIEYMKYLPGKKYDTVRSKLYTLVDFVVY |
| | SYLKYHKEGKKFSEEMVDQLRAAESDEDKNIVYCQGAERLYNIEIIRRTIKALIKDMDIKSEFKQPDK |
| | DNTYYQPIMDGMEEAKKDFITTDQLSLFTKFIYVLCQFLDGKNINILLSSLISKFQQIEAFNGDIRKLNL |
| | NIRDDGKIGYDSKKYSIFEKSGQIADDLDRLRGVIKMDINDLNAYETMIKDALRVIGVDESDIESIYQT |
| | H |
| GCA_ | MAKKKRMSAKERKQQQINLRIKKATEDSTKKVNTTVAVNNKPISKEIKKSKAKLAGVKWVIKAND |
| 900314705.1_ | DVAYISSFGKGNNSVLEKRIIGDVSSDVNKDSHMYVNPKYTKKNYEIKNGFSSGSSLTTHPNKPDKN |
| Rumen_ | SGMDALCLKTYFEKEIFKDKFNDNMHIQAIYNIFDIEKTLAKHITNIIYAVNSLDRSYIQSGNDTIGFGL |
| uncultured_ | NFNIPYAEYGGGKDSNGKPENKSAWEKRESFIKFYNNAKDRFGYFESVFYQNGKQISEEKFYIYLNIL |
| genome_ | NFVRNSTFHYNNTSSHLYKERYCKINPKNNLKTDFEFVSYLNEFVKNKFKNVNKNFISNEKNNLYIIL |
| RUG026_genomic | NAYGEDIEDVEVVKKYSKELYKLSVLKTNKNLGVNVKKLRESAIEYGYCPLPYDKEKEVAKLSSIK |
| SEQ ID NO: | HKLYKTYDFVITHYLNSNDKLLLEIVEALRLSKNDDKKENVYKIYAEKIFKAEYVINPIKTISNLFAEK |
| 5187 | GDKLFNEKVSISEEYVEDIRIDKNIHNFTKVIFFLTCFLDGKEINDLLTNIISKLQVIEDHNNVIKAIANN |
| | NDAVYKDYSDKYAVFKNSGKIATELEAIKSIARMENKINKAFKEPLLKDAMLALGVSPNDLDEKYE |
| | KYFKTDVDADKDHQKVSTFLMNNVINNSRFKYVVKYINPADINRLAKNKHLVKFVLDQIPHKQIDS |
| | YYNSVSTVEEPSYKGKIQLLTKKITGLNFYSLFENCKIPNVEKEKKKAVITLYFTIIYILVKNLVNINGL |
| | YTLALYFVERDGFFYKKICEKKDKKKTNKDVDYLLLPEIFSGSKYREETKNLKLPKEKDREIMKKYL |
| | PNDEDRKEYNKFFKQYRNNIVHLNIIANLSKLTSTIDKEINSYFEIFHYCAQRVMFDYCKNNNKVVL |
| mgm4773634.3 | LPVNEGETRKDLIGLKKQLEEVVFLKYADENHQEGQHFNDNIRIQIAYSIFDIIKVLVPISAQLIYTVN |
| SEQ ID NO: | GLDRHNDKEDAIGYYLNYSVTYENFGAVKENSSEKAKFKILEKRNKYEKFVSKAKENFIYFKDVFY |
| 5188 | EEKPAQLNNGQKTNKKSQVEYQLKSDEEIYYILNCLNFLRNNIAHFKISNQNDGNAIFRDCYYDKSA |
| | PKNLFNKGMYQVTDSAYGKAVDKINKSSVKYEDKNLYLLAKALGFAESKYSERHKELAQLIFKTSIL |
| | KSNSNRGYSnCKIRELLFEKNILTISDDEKTVNTYRAKLYKILDLVLTYYLSSSEYENMIEKFIDQLRG |
| | NQYEEEKQESYANFTEKIGEIDEIRIALKRVVSLFEGKEYANKNVIKLKEEWIDECKINKNVSDFTKLI |
| | YFISRFLSGKEINILLTQCISKLQNIDAFNKTVEELVEKGILEMHHYKRDYSLFERSGEIAKELEIVKSIS |
| | KMDRSFDHYPLEKLYKDFLLTLGVKESNKNDSENNIEDENSLEYCYEKYFSKDSKTKLSAFIKNSVIN |
| | SQRFLYVVKYVKPKDVNAIMKNPSVVYYVLQQMTEAQIERYCKTIGIDIDFELKICKRKDLLKMLIDI |
| | DFSKIRGNRSLQNKKNRGELNRNDTIEREKKKALLGLYYTVAYIFLKNMVNVNGRYILAFYCLERD |
| | KILYKEAFAKQLSTIEKSKKWSTREFYDTLTRLFTGKLNLDCQSVPLLSEKASEKLDRYIFEEPKGKSE |
| | GEKDGGDWSGYSNINDRIYVFYRNTVEHLNLIGNIGRYLEFYENVAMDCYSYDVAIGSKTKEDIKM |
| | SRVYFSFYHHMMQKMFFEEYKASKNRLINKSNNRSHIKSEKKNK |
| ULSD01.1 | MMGKHLNAKQRELEKKLKNQQKGMMYNKSTDAVSVPTLQAAPKKAAEASTLITPGTLKTKAKAM |
| SEQ ID NO: | GLKSTLVFDDKIVVTSFLNSKTEENEKCAHIEKITDCNGQTIVERPRMFNTSINAQQVDLSKDNDETN |
| 5189 | YPNPAFEDCGRDYINIKSALEKRVFGKTYNKDNLHVDIAYNIFDIKKIIGTYINNITYIFYNLGREEYN |
| | AKKDIIGNQDSDYINKLLNNTSAYFTYFDGVFKQITDRDSNKDREVKNSYNALVLKVLYYLRQFCM |
| | HGNTYTKRNEESFLSDTALYNVKDFFAKADPQINELIDAVYADGIKTINNDFMAHAKNNMYIICEVY |
| | KNEVEDSLMKEYYDFVVRKEGNNLGFNTRQLREILIDKYVGNLRDKKYDTFRNKLYTVLGFILVKEI |
| | KRNPKIQDSFIAKLRANQSGDEGKLNIYNEFAPKIWSVVSSKFNSAIKCFDEESLSKFKGYKDIDESLIS |
| | KYGITVSNTDTLVKILYFLCKFLDGKEINELCCAMINKFDNINDLIKTAAQCGEDIEFVKEYKLFINSK |
| | DLSDQIRVVKSISKMKPELSNIGEVLLLDAIDILGYKINKYKCDADGNRLVDSNNKPVYSEEYCTFKK |
| | DFFETCELDEFGRVKYDKKGKPVINHRRRNFIINNVLSSKWFFYVAKYNRPSECQKFMKSKKLIALA |
| | LKDVP |
| IMG_ | LNEIRQWTAHYESAYKIFSGSELIKEELGANKEPQNNRWMVIESYYKGLVNRINRGFLNNSALYNFP |
| 3300006226 | LLFDLVKPKDENEKKKLIEEYYKFSVLKDGKNVGVNLRTVREAMFKNGFHDVKEKEHDTYRSKFY |
| SEQ ID NO: | GIVDFLLYRSESKNFSGWSEELRKTVDKVAKEKCYDKWGESVAARMRPKVTALLDKLSKMEWKK |
| 5190 | KPNKEDLEKLAEPYKSYLADVQLSADDADPLVKLLAFLCNFWEGKEINELLSAYIHKFENIQEFIDTI |
| | EKLEGKKPTFSEKYALFNEPARRRTEEVAEIPGTRAGEIAQNLRVLASIGKMKPDLGDAKRQLYKAA |
| | IEMLGVPDDSQFVSDQWLAENVLLDKTQSGYANRKTEVNPFRNFVAKQVIESRRFMYLVRYTKPKT |
| | VRTLMENKAVVHYVLSRIDNSKNVKGKEDIITSYYKTLKEYDPQKHQHLPTDKAEREKEINRRIDAL |
| | ADYLCKYSFEKNVLKQKDGIVRNTKSATKNVEIERLKALTGLYLTVAYIAIKNLVKANARYYIAFSIF |
| | ERDYALFEKKLGRDALDERPKYPDDKGKETEADYNFFALTKYLLDKDDKAFGSWKPYQWDESKN |
| | KGENWKALRLHIKQHKKENQRHFSQKWLNFFRQQIENAKNISETGYLLTAARNHAEHLNVLTALPK |
| | FVGEFRKTEGKMTSYFELYHFLLQKLMLADAGLKNLDKYRERINKYQTSCKDLIKITYVSLGYNLA |
| | RYKNLTIEALFDSDSENGKALTKEREKKAEERAKERAKKH |
| IMG_ | MSDNKKTIAKRMGIKSVLAHGKDEQGHTKLAITAFGKGNKTDDEILKTDAKGTNLEQKHKGRNITA |
| 3300002469 | EKIVSKGIQTRGTIAREYHDTFLDTLGENLGEDYLKLKETLEKEFFGKSFPGDSVRIQIIHQILDIQKLL |
| SEQ ID NO: | GIYITDIIYCINNLRDETHLKDESDIVGLSMNDDHKLKINLGNMLPYLGFFGDAFKMPPKPKKDKSGK |
| 5191 | VINQGETPEDVHNKNVLRILGTMRQTAHFRNASLPFARDGKLANRFKKYTEEEKKNPKGKTVKMT |
| | VLKWDAWQTVEDYYAKLVDRINEGFCKNAATNVHFLTELLPXESKXQLTEDYFRFAILKEGKNLX |
| | VNMKRLREVMXALFVPELTAPETKKXYDSYRAKIYGLTDFLLFKHXHNTKQLEEWVAVLRETSNE |
| | DAKENLYDEFARTAWNTVGDSAKQLIENMQSYFTKKEKEITKTAQPVLSTSSIAHTSKKITQFSSFAK |
| | LLAFLCNFWEGKEINELLSAYIHKFENIQEFIXXXEKLEGKKPTFSEKYALFNEPARRRTEEVAEIPGT |
| | RAGEIAQNLRVLASIGKMKPDLGDAKRQLYKAAIEMLGVPDDSQFVSDQWLAENVLLDKTQSGYA |
| | NRKTEVNPFRNFVAKQVIESRRFMYLVRYTKPKTVRTLMENKAVVHYVLSRIDNSKNVKGKEDIITS |
| | YYKTLKEYDPQKHQHLPTDKAEREKEINRRIDALADYLCKYSFEKNVLKQKDGIVRNTKSATKNVEI |
| | ERLKALTGLYLTVAYIAIKNLVKANARYYIAFSIFERDYALFEKKLGRDALDERPKYPDDKGKETEA |
| | DYNFFALTKYLLDKDDKAFGSWKPYQWDESKNKGENWKALRLHIKQHKKEN |
| IMG_ | MGKTHKKNGNGSAKAHGLKMVITSNNDVSVATFGNKTKPTIIEQTLDFDTKEKIYDIDEKNFDASIE |
| 3300026549 | PIKTLKLVAKKKINNEKEFPEYNIQVNQNRKDLLGFKDKLEAEMFVGSNYKDNLHVQIAYNILDFKK |
| SEQ ID NO: | IIGLFIGDVLNSIEHLSKNPVDEVGTINVNIGYDDLSKSKKEKIDKVLKELYKYSIYFDDVFENNNSLK |
| 5192 | SDYDILRILSLIRQSVLHDSTFKNSLFTPGNNEALLDLANKAMNFVKNDFNLFKENFSSNSIVNVRILN |
| | KKLGENNWGGKYYNYVLRRDNKNIGFSITKIKTKFMEFFFNGKEDEIKTFFGKLSTLFEFLIFEYYKD |
| | ASHEIFVASIIESLRECRTEEEKEIIYEKEFQRLISENILKKELELISTIDAEYISNVKEESKGYKLNVQKS |
| | SWSFNYFPSLIYVLCKFLDQKEVSELTTSIINKLENIKSLILTAKDLKIWDCDFIPELKIFNENQINDIIDE |
| | FRVVYNLSVSKRKLKKVEPNTNNTKISKALYIDALNMFKKDDFVNNDNLSDYVQLEKTDEENALKK |
| | TKKFLINNIIKNRRFIYLLKYIDPKDCHKLVTNEKILEYIFNGEKDKYLPDSIIESYYSKITGKKINLPSR |
| | DRMTNILINLIKEIDISTVISSAFDSERIEKQKQIFTLYLTICYLIVKGLINTNSVYLLACSAYDRDAYYA |
| | FGAEEDRTMGLKLLNRYLEGERSKAKPKTRVIAYLEKNIITAKTVLKYDLKRDNLYIGKGYRDAISH |
| | FNFVNMAPSYIDRIKSVSSFFDLYNFTVQNWYLEKAKEQNIDDGYVEQLRVNLEKYGKTQKDFLKII |
| | NVPFAYNLARYKNLTIEDLFYDRYNLKGDKETSTD |
| IMG_ | MNKIHKKQGKTTAKSLGLKSVLKIENDLVVTTFGKKDNPMVVEQSINKASGEKELYVDEDQVKFDS |
| 3300019376 | SLIKEKNILSLDSIQHSNHQIIVNIDQKDASEIGMDYLRLKPELEKEFFGKTFYDNVHIQIAYNLLDLKK |
| SEQ ID NO: | IIGLHIGNAIQALENLGRDGSDLVGICDATKPLNYLDDVKQKADIGFMNRLKPYFMYFDGVLKLDNS |
| 5193 | KNKNGELNQLDIENWDVIRILSLIRQGCAHAGAYSSLLYTAQNNKVYADLINKALSIFSDDLDKFNK |
| | SFLKQSKMNLFILFDLYNCRFDRSLQEKIIKEYYRYVLYKDNKNLGFSLKNVRNLIIEGKYDEQERSG |
| | KLQTIRSKLNTLLDFYLYGYYQKNPTFVENIVAKLRESKNDEDKEKVYEEEYHRLLSENNYLVDKK |
| | CSDIVYRINEAVKNRKIFVNANINAVVEKVSCSCFPSLIYVLCKFLDGKEVNELTTAIINKLENIASLIN |
| | ALVTLKSYGGFSEQYKIFDYPNINGLIDDFRMVKNLTSTKRKLKKASGGEDRIGRQLYADAINIFKED |
| | SFVSANDEKGTGLDQYVNKFFSKDDLGARKVRNLLLNNIIKNRRFVYLIKYIDPKDCYKLVHNEKIV |
| | RFALGQYDESQMPLNQLQKYYDAVIENREGFRKCNDRKKIIDTLVSEINRVSIDGILDIGNRLVNRGN |
| | NDYINHQKQIISLYLTIAYLIVKGVVHTNSLYFIAWHAYERDNNFKFGNDGKDYLALTKEYLTNKKK |
| | RVKQLLDHNIEEANNSLDSKYFSAYRNKVVHLNFCNIFVNYLDGIGDIHSYYDIYQYVIQKWSIAERS |
| | KDFIDPQYLTKLSNDLKQYRTYQRNFLKIINLPFAYNLARYKNLTIGDLFNDKYPLPKETVKEFYNEE |
| IMG_ | MKQQRYESFSLGFVRXXXXXXXXXXXXXXXXXXXXXXXXXXXXXXXXXXXXXXXXXHGDPKQ |
| 2061766007_3 | NIYKVEGFGSHTEIANLLDRLYSERVNELNSGFLIKAKNNLILLFRAFGVKGKEQKAAFXXXXXXXX |
| SEQ ID NO: | XXXXXXXXXXXXXXXXXADIEEAFEIRKKDYYSVRGKVYPFMDFAIFRYYRNSEEASVKLVSELRA |
| 5194 | SMNEVEKEEVYRREAQKXXXXXXXXXXXXXXXXXXXXXXXKVEDKDISKEMLEDILITPDAAQFT |
| | KFVYLITLFINGKEINDLLTTLIHQFEXXXXXXXXXXXXXXXXXRGWKLSSGKSFRFSDGVKRSPGN |
| IMG_ | MNHQTRKSTSKRLGVQTIVNKNNNIFVFSNNQPKTKSVQLADEHQQLIEQNNDLDNFKTDLEYGNI |
| 3300008271 | KFGLSNNALEAFYPNQYTIRKKSSSINQWLVDKYCPTQCPDSNLILQLIHNNEDFKKIIALYSDVVFS |
| SEQ ID NO: | MKVFKGNRNEDYISKIFNNMNAKEIIDKVKSVIDSIKKRPYLFGFNPKDSASDQQIAYHLKNISMLRN |
| 5195 | YITHDSNDNLRIDSDTYASLTEQLDQFIEQENKKFTEQNGHNLALISQFYPSWSDEEVVKNYHDYYF |
| | NMLDKNLGVNLRQIALLIREKNNISQMLDDDRRAKFNVVLKFYLYTWLKANQAQLDDYIQQLRQA |
| | QLDDKDTIYQKIAQDIPANDINRLIKLCQQIDFQIKPTRFKRIPIKTINEQWDFRVLMIYSLTKFLTIKET |
| | NELISALLNKIEAMSNLLEIDRALKVQAVDAIQPNQWFFVSLPMPKKPVKNTKQPKPKHQSQSKPAN |
| | GIRGLAELRAQFQPSITHSVSSDNQLIQHQPVITSLKSIDYQRITRQLTIVNRIRSYHQPKHAEYHRQEL |
| | IDAISLFWGSDELCITDGIFDEEIKVLVNGSTKPVSRKLKNILRNNVIKTKFFNYLVRYIEPKKVANLL |
| | HNNYGLALFLVQQLTPSQLESLMIEYIPLEDENQLVGQTPREELTEAQKQRLEQYEHQLAMIFSEKIS |
| | LTYCVNRKMTVGRLALLNHI |
| IMG_ | MXXXXLKGKNPFRNFIANNVIESTRFIYVVKFCNPDKVRSLVNNTVVTKFVLGRMPETQIDRYYQSC |
| 2061766007 | IANPDSAAQLAARIDALADMMRNMRFEDFKDVQQKSGDPIENMRKERFKAIIRLYLAVVYQLVKNL |
| SEQ ID NO: | VNVNSRYVIAFHCLERDCHIYTGKSVSKSKKYFTLIDVLLEEGDSSRSGYLARNVRMREHIAHDVDI |
| 5196 | GKSLIITTKSTDSATGEVKYSKLRIIGFYRNNIAHLTAVRSFADYIGDIARIDSXXXXDYSLDRKVEDL |
| | ADMMKKMHFDAFKDVKQDTKSIKNKTEDRKERMRKERYKAMIGLYLSVVYQLVKNLVYVNSRY |
| | VMAFHAXXXXDLPRLYGADPHAVEEGSQFQKRSSLR |
| OHAC01.1 | MNIMRNAFKRANYVLCLLLALSGTSCINRLNDEIKEGSILISFSFEASKAATKVTKNTFDIGDRSGIFA |
| SEQ ID NO: | MLTGNSLDQQRYIDNLLLECSDNSKLISKKEVYYPEGDATLDFISYYPYQEENVSKGSSLLDVTVQA |
| 5197 | DQNKTANYSLSNFMTARIGNVPNSEKTIKLEYKHRFAKIKLVLIPQEGEDTDEMLKANPRIIATGFRT |
| | QAVYDLQSDKLSSVDDASETDIIPFGTWKKEGNTLSGKEFIVIPQTHSDGGQAFTLEWNGKIYPCPLP |
| | SATIEEDTELEICINALQSTSATLTGVIANIKEWELSEQGESENRYEITAVHTASLSFSTSDIYRIYHQGK |
| | PVAEVCREYLYTAPADAVATKAIVAYSVQDNEQTDLTNGIVLQLPDKTATTHGGKVSWNEADNSL |
| | TYISGHSRPIEKFYIDENRKIVTEKPAAPALAINVSSYVIRDIRNGILHTYPIVKIGAQYWMKEDLQTA |
| | HYNDSKSMPLRKALGDGEGYLKWAGTNSHFYNGEAVLTGKLAPLDWRLPTENDWNRLKEYIGDIR |
| | TVDSYFSIYHYVMQRCITKRENTWSSDVYSATNETGFCIQPAGLLLERENKTALVNANSSTAYWLY |
| | DGTQKQLDKVVMFANSNNDIALKNAVKPEGKDYYNAFSIRCIKE |
| OJKY01.1 | VYINSRYALAFAKYEHDASLLLDGWVYDRYKTVFSDLTVDSLQKGKLNRRASKYLQVNLEHSDTD |
| SEQ ID NO: | LIRQYRNKVAHLNAMFDLMSKVYVDATMQGKKGMNEHSALVSMIDRSNIPGKVIVLMDRGYESFN |
| 5198 | NIAHLQEKEWNFIIRAKESYGMISNLQLPNSEEFDVDTTLTLTRRQTKETLALLSAYPERYRWIQPHT |
| | TFDYIAPKAPNMYDLHFRVVRFRISDGCYETIYTDLDPETFPVEKIKELYRLRWGIETSFRELKYISGL |
| | SCLHGKKADFLLQEVFARLIFYNYASLIARQVPAPQGKQINFSVAILACKQFLKGKIRSAQIFEILSMH |
| | LSPIRPGRQYKRYQNPVSAVAFQYRLS |
| OMBP01.1 | VYINSRYALAFAKYEHDASLLLDGWVYDRYKTVFSDLTVDSLQKGKLNRRASKYLQVNLEHSDTD |
| SEQ ID NO: | LIRQYRNKVAHLNAMFDLMSKVYVDATMQGKKGMNEHSALVSMIDRSNIPGKVIVLMDRGYESFN |
| 5199 | NIAHLQEKEWNFIIRAKESYGMISNLQLPNSEEFDVDTTLTLTRRQTKETLALLSAYPERYRWIQPHT |
| | TFDYIAPKAPNMYDLHFRVVRFRISDGCYETIYTDLDPETFPVEKIKELYRLRWGIETSFRELKYISGL |
| | SCLHGKKADFLLQEVFARLILYNYASLIARKIPVPQEKQINFSVAILVCKQFLKGKIRPAQIFEILSKYL |
| | SPIRPGRQYRRYQNPVSAVAFQYRLS |
| ORQL01.1 | VYINSRYALAFAKYEHDASLLLDGWVYDRYKTVFSDLTVDSLQKGKLNRRASKYLQVNLEHSDTD |
| SEQ ID NO: | LIRQYRNKVAHLNAMFDLMSKVYVDATMQGKKGMNEHSALVSMIDRSNIPGKVIVLMDRGYESFN |
| 5200 | NIAHLQEKKWNYIIRAKESYGMISNLQLPNSEEFDVDTTLTLTRRQTKETLALLSAYPERYRWIQPHT |
| | TFDYIAPKDPAMYDLRFRVVRFRISGGCYETVYTNLDSETFPIGKIKELYRLRWGIETSFRELKYTIGL |
| | SCLHGKKTDFLLQEVFARLILYNYASLIARKIPVPQEKQINFSVAILVCKQFLKGKIRPAQIFGILSKHL |
| | SPIRPGRQYKRYQNPVSAVAFQYRFS |
| OWST01.1 | MRFSCAFFLEQQMRDGADDQQADDSTLKEDHEQLPDVERQLGAEDQTADLREVEDLRDGDDRDD |
| SEQ ID NO: | EAGHVARTGARDQDGRGKHIGKADDHRGSGSGGIRHLQQLEQGQEGRAEDLEDVGVVGADQRNG |
| 5201 | ADAHNGGQDAEPHITGVLCAVLKEPADAGEVRNALEREVHAVLAGALVALGDGLRRSFALDHIGL |
| | GLFVQGPQQRADQRADDADEADEQVVNAEVLHHPDLIEHDQRKGDGNGGEERRDDARLLSGVVA |
| | ALILVEGQHDTERAVADPRGHAVDRGAAGDLEERAHELRRQRADILEQTEVGQQRQQEGRDHIDD |
| | DQRADQIIEHETALIGAGDGIEDAAPLVGAQLKKCDPREDRTQHTHDDPERTADEAAVKYGAFHDE |
| | LGLCQRHERHHSGDRHDRHAQNRQESTDRTGNNVRDDLDRDRPLACRVFDRDHKENNTDRGRND |
| | KHRFQFTSVFHILPPLIVEHIKMFLFSSHIFICSQQTSYLSFPEGQVPVRPLPCVPHGICRPAEQARQSLS |
| | SLSKLSLEDFRNVEQSANAARNAQKAKYQGMLSLYLNVIYQIIKNMVYINSRYALAFAKYEHDASL |
| | LLDGWVYDRYKTVFSDLTVDSLQKGKLNRRASKYLQVNLEHSDTDLIRQYRNKVAHLNAMFDLM |
| | SKVYVDATMQGKKGMNEHSALVSMIDRSNIPGKVIVLMDTQKGRCKPNLEQHLSEYDLSLHPTHR |
| | GIRI |
| UPMX01.1 | VPHGICRPAEQARQSLSSLSKLSLEDFRNVEQSANAARNAQKAKYQGMLSLYLNVIYQIIKNMVYIN |
| SEQ ID NO: | SRYVLAFAKYEHDASLLLDGWVYDRYKTVFSDLTVGSLQKGKLNRRASKYLQVNLEHSDTDLIRQ |
| 5202 | YRNKVAHLNAMFDLMSKYYPNIAELSKDEVAKQAPFGAKWRLLAQVEVRCYILISNISGSVKVAPL |
| | QILQFPVILPHKFQDAGKFNLQFSDFSEITETADKLRWLRYQKGLRQRDVADYAGIYRSTYIHYEEYG |
| | KDFYSPKHMEKIAQLFEVPVERLLDDYNLFLRNGQGKQIKAIRMKLGLTQREYADRLGISLCNLKQ |
| | WEQNRKQLFKSTWEKYFK |
| IMG_206176 | MEINSRTTPPSRGLKLYELFVLLESQHRSHYTPVAGSETHQYPCIYGRLSWSHYTPVAGSETTEAADL |
| 6007 8 | SHKDIMSHYTPVAGSETFSYYPSISAKICRTTPPSRQCHQFKGXXXXXXXXXXXXXXXXXXXXXXX |
| SEQ ID NO: | XXXXXXXXXXXXXXXXXXXXXXXXXXXXXXXXXXXXXXXXXXXXXXXXXXXXXXXXXXXXX |
| 5203 | XXXXXXXXXXXXXXXXXXXXXXXXXXXXXXXXXXXXXXXXXXXXXXXXXXXXXXXXXXXXX |
| | XXXXXXXXXXXXXXXXXXXXXXXXXXXXXXXXXXXXXXXXXXXXXXXXXXXXXXXXXXXXX |
| | XXXXXXXXXXXXXXXXXXXXXXXXXXXXXXXXXXXXXXXXXXXXXXXXXXXXXXXXXXXXX |
| | XXXXXXXXXXXXXXXXXXXXXXXXXXXXXXXXXXXXXXXXXXXXXXXXXXXXXXXXXXXXX |
| | XXXXXXXXXXXXXXXXXXXXXXXXXXXXXXXXXXXDGKEINILFTTLINKFENIASFITTAKELSI |
| | DVTFHENYAFFNQDCDGYAREIDVVRNIARMKKPVPGARKTMYRDALTVLGIPEHMQADMFDAEL |
| | EKMLEKPKDEKGRKLKGKNPFRNFIANNVIESNRFIYVXXXXERQEPLPQLHRQQRHRIHPVYLCGEI |
| | LQSR |

### Cas 13 Variants

The Cas proteins herein include variants and mutated forms of Cas proteins (comparing to wildtype or naturally occurring Cas proteins). In some examples, the present disclosure includes variants and mutated forms of the Cas proteins. The variants or mutated forms of Cas protein may be catalytically inactive, e.g., have no or reduced nuclease activity compared to a corresponding wildtype. In certain examples, the variants or mutated forms of Cas protein have nickase activity.

In some cases, the present disclosure provides for mutated Cas13 proteins comprising one or more modified of amino acids, wherein the amino acids: (a) interact with a guide RNA that forms a complex with the mutated Cas 13 protein; (b) are in a HEPN active site, an inter-domain linker domain, or a bridge helix domain of the mutated Cas 13 protein; or a combination thereof.

The term “corresponding amino acid” or “residue which corresponds to” refers to a particular amino acid or analogue thereof in a Cas13 homolog or ortholog that is identical or functionally equivalent to an amino acid in reference Cas protein. Accordingly, as used herein, referral to an “amino acid position corresponding to amino acid position [X]” of a specified Cas 13 protein represents referral to a collection of equivalent positions in other recognized Cas 13 and structural homologs and families. The mutations described herein apply to all Cas13 protein that is orthologs or homologs of the referred Cas protein (e.g., PbCas13b). For example, the mutations apply to Cas13a, Cas13b, Cas13c, Cas13d, e.g., SEQ ID NOs. 1-4092, 4102-5203, and 5260-5265.

In an aspect, the invention relates to a mutated Cas13 protein comprising one or more mutation of an amino acid corresponding to the following amino acids of Prevotella buccae Cas13b (PbCas13b): T405, H407, K457, H500, K570, K590, N634, R638, N652, N653, K655, S658, K741, K744, N756, S757, R762, R791, K846, K857, K870, R877, K183, K193, R600, K607, K612, R614, K617, K826, K828, K829, R824, R830, Q831, K835, K836, R838, R618, D434, K431, R53, K943, R1041, Y164, R285, R287, K292, E296, N297, Q646, N647, R402, K393, N653, N652, R482, N480, D396, E397, D398, E399, K294, E400, R56, N157, H161, H452, N455, K484, N486, G566, H567, A656, V795, A796, W842, K871, E873, R874, R1068, N1069, or H1073.

PbCas13b as used herein preferably has the sequence of NCBI Reference Sequence WP_004343973.1. It is to be understood that WP_004343973.1 refers to the wild type (i.e. unmutated) PbCas13b. LshCas13a (Leptotrichia shahii Cas13a) as used herein preferably has the sequence of NCBI Reference Sequence WP_018451595.1. It is to be understood that WP_018451595.1 refers to the wild type (i.e. unmutated) LshCas13b. Pgu Cas13b (Porphyromonas gulae Cas13b) as used herein preferably has the sequence of NCBI Reference Sequence WP_039434803.1. It is to be understood that WP_039434803.1 refers to the wild type (i.e. unmutated) Pgu Cas13b. Psp Cas13b (Prevotella sp. P5-125 Cas13b) as used herein preferably has the sequence of NCBI Reference Sequence WP_044065294.1. It is to be understood that WP_044065294.1 refers to the wild type (i.e. unmutated) Psp Cas13b.

In embodiments of the invention, a Type VI system comprises a mutated Cas13 effector protein according to the invention as described herein (and optionally a small accessory protein encoded upstream or downstream of a Cas13 protein). In certain embodiments, the small accessory protein enhances the Cas13's ability to target RNA. Insights from the structure of Cas13 enables further rational engineering to improve functionality for RNA targeting specificity, base editing, and nucleic acid detection, etc. Based on the elucidated crystal structure of the Cas13 effector with its crRNA described herein, functional implications of rational engineering and mutagenesis can be postulated, of which non-limiting mutations are exemplified in Table 6 below (with reference to PbCas13b; WP 004343973.1).

TABLE 6

| Residue | Descrption | Expected result |
|---|---|---|
| T405 | coordinates first base of guide | alter activity |
| | (U) | |
| H407 | basestacking with U0 | possible PFS |
| | | involvment |
| H407Y/W/F | basestacking with U0 | alter PFS |
| K457 | direct readout of A31 | |
| H500 | hydrogen bond with bb of G11 | alter activity |
| K570 | direct readout of G25 | alter activity |
| K590 | bb of U27 | alter activity |
| N634 | bb of A29 | alter activity |
| R638 | bb of A28 | alter activity |
| N652 | direct readout of U2 and C36 | alter activity |
| N653 | direct readout of C36 | alter activity |
| K655 | hydrogen bonds with bb of na 3 | alter activity |
| S658 | coordinates first base of guide | alter activity |
| K741 | direct readout of U27 | alter activity |
| K744 | hydrogen bonds with bb of na 6 | alter activity |
| N756 | direct readout of C33 and C5 | alter activity |
| S757 | direct readout of A32 | alter activity |
| R762 | hydrogen bond with bb of G10 | alter activity |
| R791 | bb of A22 | alter activity |
| K846 | hydrogen bond with bb of U18 | alter activity |
| K857 | hydrogen bond with bb of C15 | alter activity |
| K870 | hydrogen bond with base of | alter activity |
| | U19 | |
| R877 | direct readout of U18 | alter activity |
| | Channels | |
| K183 | Outerchannel rim | alter activity |
| K193 | Outerchannel rim | alter activity |
| R600 | Outerchannel rim | alter activity |
| K607 | Outerchannel rim | alter activity |
| K612 | Outerchannel rim | alter activity |
| R614 | Outerchannel rim | alter activity |
| K617 | Outerchannel rim | alter activity |
| K826 | Bridge helix domain | alter activity |
| K828 | Bridge helix domain | alter activity |
| K829 | Bridge helix domain | alter activity |
| R824 | Bridge helix domain | alter activity |
| R830 | Bridge helix domain | alter activity |
| Q831 | Bridge helix domain | alter activity |
| K835 | Bridge helix domain | alter activity |
| K836 | Bridge helix domain | alter activity |
| R838 | Bridge helix domain | alter activity |
| R618 | conserved outer channel | alter activity |
| | arginien | |
| D434 | Conserved loop | alter activity |
| K431 | Conserved loop | alter activity |
| | Active site pocket | |
| 46-57 | HEP1 | |
| 73-79 | HEP1 | |
| 152-164 | HEP1 | |
| 1036-1046 | HEP2 | |
| 1064-1074 | HEP2 | |
| R53A/K/D/E | HEP1 | change in base |
| | | specificity |
| K943A/R/D/E | HEP2 | change in base |
| | | specificity |
| R1041A/K/D/E | HEP2 | change in base |
| | | specificity |
| Y164A/FAV | | affect base stacking at |
| | | active site |
| | Interdomain linker | |
| 285-299 | | |
| R285 | central channel active pocket | alter activity |
| R287 | central channel active pocket | alter activity |
| K292 | central channel active pocket | alter activity |
| E296 | central channel active pocket | alter activity |
| N297 | central channel active pocket | alter activity |
| | Other | |
| | Trans active site loop | alter activity |
| Q646 | Trans active site loop | alter activity |
| N647 | Trans active site loop | alter activity |
| | HEPN interface | |
| | crRNA processiong | |
| R402 | remove crRNA processing | alter crRNA processing |
| K393 | remove crRNA processing | alter crRNA processing |
| N653 | remove crRNA processing | alter crRNA processing |
| N652 | remove crRNA processing | alter crRNA processing |
| R482 | remove crRNA processing | alter crRNA processing |
| N480 | remove crRNA processing | alter crRNA processing |
| | LID domain | |
| D396 | hairpin with unknown function | alter crRNA processing |
| E397 | hairpin with unknown function | alter crRNA processing |
| D398 | hairpin with unknown function | alter crRNA processing |
| E399 | hairpin with unknown function | alter crRNA processing |
| K294 | IDL | alter activity |

The Cas13 protein herein may comprise one or more mutations. In some cases, the Cas13 protein comprises one or more mutations of amino acid corresponding to the following amino acids of Prevotella buccae Cas13b (PbCas13b): T405, H407, K457, H500, K570, K590, N634, R638, N652, N653, K655, S658, K741, K744, N756, S757, R762, R791, K846, K857, K870, R877, K183, K193, R600, K607, K612, R614, K617, K826, K828, K829, R824, R830, Q831, K835, K836, R838, R618, D434, K431, R53, K943, R1041, Y164, R285, R287, K292, E296, N297, Q646, N647, R402, K393, N653, N652, R482, N480, D396, E397, D398, E399, K294, E400, R56, N157, H161, H452, N455, K484, N486, G566, H567, A656, V795, A796, W842, K871, E873, R874, R1068, N1069, or H1073.

In some cases, the Cas13 protein comprises one or more mutations of an amino acid corresponding to the following amino acids of Prevotella buccae Cas13b (PbCas13b): H407, K457, H500, K570, K590, N634, R638, N652, N653, K655, S658, K741, K744, N756, S757, R762, R791, K846, K857, K870, R877, K183, K193, R600, K607, K612, R614, K617, K826, K828, K829, R824, R830, Q831, K835, K836, R838, R618, D434, K431, R53, K943, R1041, Y164, R285, R287, K292, E296, N297, Q646, N647, R402, K393, N653, N652, R482, N480, D396, E397, D398, E399, K294, E400, R56, N157, H161, H452, N455, K484, N486, G566, H567, W842, K871, E873, R874, R1068, N1069, or H1073.

In some cases, the Cas13 protein comprises one or more mutations of an amino acid corresponding to the following amino acids of Prevotella buccae Cas13b (PbCas13b): T405, H407, K457, H500, K570, K590, N634, R638, N652, N653, K655, S658, K741, K744, N756, S757, R762, R791, K846, K857, K870, R877, K183, K193, R600, K607, K612, R614, K617, K826, K828, K829, R824, R830, Q831, K835, K836, R838, R618, D434, K431, R53, K943, R1041, Y164, R285, R287, K292, E296, N297, Q646, N647, R402, K393, N653, N652, R482, N480, D396, E397, D398, E399, K294, or E400.

In some cases, the Cas13 protein comprises one or more mutations of an amino acid corresponding to the following amino acids of Prevotella buccae Cas13b (PbCas13b): K393, R402, N482, T405, H407, S658, N653, A656, K655, N652, H567, N455, H500, K871, K857, K870, W842, E873, R877, K846, R874, R762, V795, A796, R791, G566, K590, R638, H452, S757, N756, N486, K484, N480, K457, K741, R56, N157, H161, R1068, N1069, or H1073. In some cases, the Cas13 protein comprises one or more mutations of an amino acid corresponding to the following amino acids of PbCas13b: K393, R402, N482, H407, S658, N653, K655, N652, H567, N455, H500, K871, K857, K870, W842, E873, R877, K846, R874, R762, R791, G566, K590, R638, H452, S757, N756, N486, K484, N480, K457, K741, R56, N157, H161, R1068, N1069, or H1073.

In some cases, the Cas13 protein comprises one or more mutations of an amino acid corresponding to the following amino acids of PbCas13b: W842, K846, K870, E873, or R877. In some cases, the Cas13 protein comprises in helical domain 1 one or more mutations of an amino acid corresponding to the following amino acids in helical domain 1 of PbCas13b: W842, K846, K870, E873, or R877. In some cases, the Cas13 protein comprises in helical domain 1-3 one or more mutations of an amino acid corresponding to the following amino acids in helical domain 1-3 of PbCas13b: W842, K846, K870, E873, or R877. In some cases, the Cas13 protein comprises in the helical bridge domain one or more mutations of an amino acid corresponding to the following amino acids in the helical bridge domain of PbCas13b: W842, K846, K870, E873, or R877. In some cases, the Cas13 protein comprises one or more mutations of an amino acid corresponding to the following amino acids of PbCas13b: K393, R402, N480, N482, N652, or N653. In some cases, the Cas13 protein comprises one or more mutations of an amino acid corresponding to the following amino acids of PbCas13b: K393, R402, N480, or N482. In some cases, the Cas13 protein comprises in the LID domain one or more mutations of an amino acid corresponding to the following amino acids in the LID domain of PbCas13b: K393, R402, N480, or N482. In some cases, the Cas13 protein comprises one or more mutations of an amino acid corresponding to the following amino acids of PbCas13b: N652 or N653. In some cases, the Cas13 protein comprises in helical domain 2 one or more mutations of an amino acid corresponding to the following amino acids in helical domain 2 of PbCas13b: N652 or N653.

In some cases, the Cas13 protein comprises one or more mutations of an amino acid corresponding to the following amino acids of PbCas13b: T405, H407, S658, N653, A656, K655, N652, H567, N455, H500, K871, K857, K870, W842, E873, R877, K846, R874, R762, V795, A796, R791, G566, K590, R638, H452, S757, N756, N486, K484, N480, K457, or K741. In some cases, the Cas13 protein comprises one or more mutations of an amino acid corresponding to the following amino acids of PbCas13b: H407, S658, N653, K655, N652, H567, N455, H500, K871, K857, K870, W842, E873, R877, K846, R874, R762, R791, G566, K590, R638, H452, S757, N756, N486, K484, N480, K457, or K741. In some cases, the Cas13 protein comprises one or more mutations of an amino acid corresponding to the following amino acids of PbCas13b: S658, N653, A656, K655, N652, H567, H500, K871, K857, K870, W842, E873, R877, K846, R874, R762, V795, A796, R791, G566, K590, R638, S757, N756, or K741. In some cases, the Cas13 protein comprises in a helical domain one or more mutations of an amino acid corresponding to the following amino acids in a helical domain of PbCas13b: S658, N653, A656, K655, N652, H567, H500, K871, K857, K870, W842, E873, R877, K846, R874, R762, V795, A796, R791, G566, K590, R638, S757, N756, or K741.

In some cases, the Cas13 protein comprises one or more mutations of an amino acid corresponding to the following amino acids of PbCas13b: H567, H500, K871, K857, K870, W842, E873, R877, K846, R874, R762, V795, A796, R791, G566, S757, or N756. In some cases, the Cas13 protein comprises in helical domain 1 one or more mutations of an amino acid corresponding to the following amino acids in helical domain 1 of PbCas13b: H567, H500, K871, K857, K870, W842, E873, R877, K846, R874, R762, V795, A796, R791, G566, S757, or N756. In some cases, the Cas13 protein comprises one or more mutations of an amino acid corresponding to the following amino acids of PbCas13b: H567, H500, R762, V795, A796, R791, G566, S757, or N756. In some cases, the Cas13 protein comprises in helical domain 1 one or more mutations of an amino acid corresponding to the following amino acids in helical domain 1 of PbCas13b: H567, H500, R762, V795, A796, R791, G566, S757, or N756.

In some cases, the Cas13 protein comprises one or more mutations of an amino acid corresponding to the following amino acids of PbCas13b: K871, K857, K870, W842, E873, R877, K846, or R874. In some cases, the Cas13 protein comprises in the helical bridge domain one or more mutations of an amino acid corresponding to the following amino acids in the helical bridge domain of PbCas13b: K871, K857, K870, W842, E873, R877, K846, or R874. In some cases, the Cas13 protein comprises one or more mutations of an amino acid corresponding to the following amino acids of PbCas13b: H567, H500, or G566. In some cases, the Cas13 protein comprises in helical domain 1-2 one or more mutations of an amino acid corresponding to the following amino acids in helical domain 1-2 of PbCas13b: H567, H500, or G566. In some cases, the Cas13 protein comprises one or more mutations of an amino acid corresponding to the following amino acids of PbCas13b: K871, K857, K870, W842, E873, R877, K846, R874, R762, V795, A796, R791, S757, or N756. In some cases, the Cas13 protein comprises in helical domain 1-3 one or more mutation of an amino acid corresponding to the following amino acids in helical domain 1-3 of PbCas13b: K871, K857, K870, W842, E873, R877, K846, R874, R762, V795, A796, R791, S757, or N756. In some cases, the Cas13 protein comprises one or more mutations of an amino acid corresponding to the following amino acids of PbCas13b: R762, V795, A796, R791, S757, or N756. In some cases, the Cas13 protein comprises in helical domain 1-3 one or more mutation of an amino acid corresponding to the following amino acids in helical domain 1-3 of PbCas13b: R762, V795, A796, R791, S757, or N756. In some cases, the Cas13 protein comprises one or more mutations of an amino acid corresponding to the following amino acids of PbCas13b: S658, N653, A656, K655, N652, K590, R638, or K741. In some cases, the Cas13 protein comprises in helical domain 2 one or more mutations of an amino acid corresponding to the following amino acids in helical domain 2 of PbCas13b: S658, N653, A656, K655, N652, K590, R638, or K741. In some cases, the Cas13 protein comprises one or more mutations of an amino acid corresponding to the following amino acids of PbCas13b: T405, H407, N486, K484, N480, H452, N455, or K457. In some cases, the Cas13 protein comprises in the LID domain one or more mutations of an amino acid corresponding to the following amino acids in the LID domain of PbCas13b: T405, H407, N486, K484, N480, H452, N455, or K457. In some cases, the Cas13 protein comprises one or more mutations of an amino acid corresponding to the following amino acids of PbCas13b: S658, N653, K655, N652, H567, H500, K871, K857, K870, W842, E873, R877, K846, R874, R762, R791, G566, K590, R638, S757, N756, or K741.

In some cases, the Cas13 protein comprises in a helical domain one or more mutations of an amino acid corresponding to the following amino acids in a helical domain of PbCas13b: S658, N653, K655, N652, H567, H500, K871, K857, K870, W842, E873, R877, K846, R874, R762, R791, G566, K590, R638, S757, N756, or K741. In some cases, the Cas13 protein comprises one or more mutations of an amino acid corresponding to the following amino acids of PbCas13b: H567, H500, K871, K857, K870, W842, E873, R877, K846, R874, R762, R791, G566, S757, or N756. In some cases, the Cas13 protein comprises in helical domain 1 one or more mutations of an amino acid corresponding to the following amino acids in helical domain 1 of PbCas13b: H567, H500, K871, K857, K870, W842, E873, R877, K846, R874, R762, R791, G566, S757, or N756. In some cases, the Cas13 protein comprises one or more mutations of an amino acid corresponding to the following amino acids of PbCas13b: H567, H500, R762, R791, G566, S757, or N756. In some cases, the Cas13 protein comprises in helical domain 1 one or more mutations of an amino acid corresponding to the following amino acids in helical domain 1 of PbCas13b: H567, H500, R762, R791, G566, S757, or N756. In some cases, the Cas13 protein comprises one or more mutations of an amino acid corresponding to the following amino acids of PbCas13b: K871, K857, K870, W842, E873, R877, K846, R874, R762, R791, S757, or N756. In some cases, the Cas13 protein comprises in helical domain 1-3 one or more mutations of an amino acid corresponding to the following amino acids in helical domain 1-3 of PbCas13b: K871, K857, K870, W842, E873, R877, K846, R874, R762, R791, S757, or N756. In some cases, the Cas13 protein comprises one or more mutations of an amino acid corresponding to the following amino acids of PbCas13b: R762, R791, S757, or N756. In some cases, the Cas13 protein comprises in helical domain 1-3 one or more mutations of an amino acid corresponding to the following amino acids in helical domain 1-3 of PbCas13b: R762, R791, S757, or N756.

In some cases, the Cas13 protein comprises one or more mutations of an amino acid corresponding to the following amino acids of PbCas13b: S658, N653, K655, N652, K590, R638, or K741. In some cases, the Cas13 protein comprises in helical domain 2 one or more mutations of an amino acid corresponding to the following amino acids in helical domain 2 of PbCas13b: S658, N653, K655, N652, K590, R638, or K741.

In some cases, the Cas13 protein comprises one or more mutations of an amino acid corresponding to the following amino acids of PbCas13b: H407, N486, K484, N480, H452, N455, or K457. In some cases, the Cas13 protein comprises in the LID domain one or more mutations of an amino acid corresponding to the following amino acids in the LID domain of PbCas13b: H407, N486, K484, N480, H452, N455, or K457.

In some cases, the Cas13 protein comprises one or more mutations of an amino acid corresponding to the following amino acids of PbCas13b: R56, N157, H161, R1068, N1069, or H1073. In some cases, the Cas13 protein comprises in a HEPN domain one or more mutations of an amino acid corresponding to the following amino acids in a HEPN domain of PbCas13b: R56, N157, H161, R1068, N1069, or H1073.

In some cases, the Cas13 protein comprises one or more mutations of an amino acid corresponding to the following amino acids of PbCas13b: R56, N157, or H161. In some cases, the Cas13 protein comprises in HEPN domain 1 one or more mutations of an amino acid corresponding to the following amino acids in HEPN domain 1 of PbCas13b: R56, N157, or H161. In some cases, the Cas13 protein comprises one or more mutations of an amino acid corresponding to the following amino acids of PbCas13b: R1068, N1069, or H1073. In some cases, the Cas13 protein comprises in HEPN domain 2 one or more mutations of an amino acid corresponding to the following amino acids in HEPN domain 2 of PbCas13b: R1068, N1069, or H1073.

In some cases, the Cas13 protein comprises one or more mutations of an amino acid corresponding to the following amino acids of PbCas13b: K393, R402, N482, T405, H407, N486, K484, N480, H452, N455, or K457. In some cases, the Cas13 protein comprises in the LID domain one or more mutations of an amino acid corresponding to the following amino acids in the LID domain of PbCas13b: K393, R402, N482, T405, H407, N486, K484, N480, H452, N455, or K457. In some cases, the Cas13 protein comprises one or more mutations of an amino acid corresponding to the following amino acids of PbCas13b: K393, R402, N482, H407, N486, K484, N480, H452, N455, or K457. In some cases, the Cas13 protein comprises in the LID domain one or more mutations of an amino acid corresponding to the following amino acids in the LID domain of PbCas13b: K393, R402, N482, H407, N486, K484, N480, H452, N455, or K457.

In some cases, the Cas13 protein comprises one or more mutations of an amino acid corresponding to the following amino acids of PbCas13b: T405, H407, S658, N653, A656, K655, N652, H567, N455, H500, K871, K857, K870, W842, E873, R877, K846, R874, R762, V795, A796, R791, G566, K590, R638, H452, S757, N756, N486, K484, N480, K457, K741, K393, R402, or N482. In some cases, the Cas13 protein comprises one or more mutations of an amino acid corresponding to the following amino acids of PbCas13b: H407, S658, N653, K655, N652, H567, N455, H500, K871, K857, K870, W842, E873, R877, K846, R874, R762, R791, G566, K590, R638, H452, S757, N756, N486, K484, N480, K457, K741, K393, R402, or N482.

In some cases, the Cas13 protein comprises one or more mutations of an amino acid corresponding to the following amino acids of PbCas13b: S658, N653, A656, K655, N652, H567, N455, H500, K871, K857, K870, W842, E873, R877, K846, R874, R762, V795, A796, R791, G566, K590, R638, H452, S757, N756, N486, K484, N480, K457, or K741. In some cases, the Cas13 protein comprises one or more mutations of an amino acid corresponding to the following amino acids of PbCas13b: S658, N653, K655, N652, H567, N455, H500, K871, K857, K870, W842, E873, R877, K846, R874, R762, R791, G566, K590, R638, H452, S757, N756, N486, K484, N480, K457, or K741.

In some cases, the Cas13 protein comprises one or more mutations of an amino acid corresponding to the following amino acids of PbCas13b: N486, K484, N480, H452, N455, or K457. In some cases, the Cas13 protein comprises in the LID domain one or more mutations of an amino acid corresponding to the following amino acids in the LID domain of PbCas13b: N486, K484, N480, H452, N455, or K457. In some cases, the Cas13 protein comprises one or more mutations of an amino acid corresponding to the following amino acids of PbCas13b: K393, R402, N482, N486, K484, N480, H452, N455, or K457. In some cases, the Cas13 protein comprises in the LID domain one or more mutations of an amino acid corresponding to the following amino acids in the LID domain of PbCas13b: K393, R402, N482, N486, K484, N480, H452, N455, or K457.

In some cases, the Cas13 protein comprises one or more mutations of an amino acid corresponding to the following amino acids of PbCas13b: S658, N653, A656, K655, N652, H567, N455, H500, K871, K857, K870, W842, E873, R877, K846, R874, R762, V795, A796, R791, G566, K590, R638, H452, S757, N756, N486, K484, N480, K457, K741, K393, R402, or N482. In some cases, the Cas13 protein comprises one or more mutations of an amino acid corresponding to the following amino acids of PbCas13b: S658, N653, K655, N652, H567, N455, H500, K871, K857, K870, W842, E873, R877, K846, R874, R762, R791, G566, K590, R638, H452, S757, N756, N486, K484, N480, K457, K741, K393, R402, or N482.

In some cases, the Cas13 protein comprises one or more mutations of an amino acid corresponding to the following amino acids of Prevotella buccae Cas13b (PbCas13b): R53, Y164, K943, or R1041. In some cases, the Cas13 protein comprises one or more mutations of an amino acid corresponding to the following amino acids of Prevotella buccae Cas13b (PbCas13b): R53 or Y164.

In some cases, the Cas13 protein comprises one or more mutations of an amino acid corresponding to the following amino acids of Prevotella buccae Cas13b (PbCas13b): K943 or R1041. In some cases, the Cas13 protein comprises in a HEPN domain one or more mutations of an amino acid corresponding to the following amino acids in a HEPN domain of Prevotella buccae Cas13b (PbCas13b): R53, Y164, K943, or R1041. In some cases, the Cas13 protein comprises in HEPN domain 1 one or more mutations of an amino acid corresponding to the following amino acids in HEPN domain 1 of Prevotella buccae Cas13b (PbCas13b): R53 or Y164. In some cases, the Cas13 protein comprises in HEPN domain 2 one or more mutations of an amino acid corresponding to the following amino acids in HEPN domain 2 of Prevotella buccae Cas13b (PbCas13b): K943 or R1041.

In some cases, the Cas13 protein comprises one or more mutations of an amino acid corresponding to the following amino acids of Prevotella buccae Cas13b (PbCas13b): R53, Y164, K943, R1041, R56, N157, H161, R1068, N1069, or H1073.

In some cases, the Cas13 protein comprises one or more mutations of an amino acid corresponding to the following amino acids of Prevotella buccae Cas13b (PbCas13b): R53, Y164, R56, N157, or H161. In some cases, the Cas13 protein comprises one or more mutations of an amino acid corresponding to the following amino acids of Prevotella buccae Cas13b (PbCas13b): K943, R1041, R1068, N1069, or H1073. In some cases, the Cas13 protein comprises in a HEPN domain one or more mutations of an amino acid corresponding to the following amino acids in a HEPN domain of Prevotella buccae Cas13b (PbCas13b): R53, Y164, K943, R1041, R56, N157, H161, R1068, N1069, or H1073. In some cases, the Cas13 protein comprises in HEPN domain 1 one or more mutations of an amino acid corresponding to the following amino acids in HEPN domain 1 of Prevotella buccae Cas13b (PbCas13b): R53, Y164, R56, N157, or H161. In some cases, the Cas13 protein comprises in HEPN domain 2 one or more mutations of an amino acid corresponding to the following amino acids in HEPN domain 2 of Prevotella buccae Cas13b (PbCas13b): K943, R1041, R1068, N1069, or H1073.

In some cases, the Cas13 protein comprises one or more mutations of an amino acid corresponding to the following amino acids of Prevotella buccae Cas13b (PbCas13b): R53, Y164, K183, K193, K943, or R1041. In some cases, the Cas13 protein comprises one or more mutations of an amino acid corresponding to the following amino acids of Prevotella buccae Cas13b (PbCas13b): R53, Y164, K183, or K193. In some cases, the Cas13 protein comprises one or more mutations of an amino acid corresponding to the following amino acids of Prevotella buccae Cas13b (PbCas13b): K943 or R1041. In some cases, the Cas13 protein comprises in a HEPN domain one or more mutations of an amino acid corresponding to the following amino acids in a HEPN domain of Prevotella buccae Cas13b (PbCas13b): R53, Y164, K183, K193, K943, or R1041.

In some cases, the Cas13 protein comprises in HEPN domain 1 one or more mutations of an amino acid corresponding to the following amino acids in HEPN domain 1 of Prevotella buccae Cas13b (PbCas13b): R53, Y164, K183, or K193. In some cases, the Cas13 protein comprises in HEPN domain 2 one or more mutations of an amino acid corresponding to the following amino acids in HEPN domain 2 of Prevotella buccae Cas13b (PbCas13b): K943 or R1041. In some cases, the Cas13 protein comprises one or more mutations of an amino acid corresponding to the following amino acids of Prevotella buccae Cas13b (PbCas13b): R53, Y164, K183, K193, K943, R1041, R56, N157, H161, R1068, N1069, or H1073. In some cases, the Cas13 protein comprises one or more mutations of an amino acid corresponding to the following amino acids of Prevotella buccae Cas13b (PbCas13b): R53, Y164, K183, K193, R56, N157, or H161. In some cases, the Cas13 protein comprises one or more mutations of an amino acid corresponding to the following amino acids of Prevotella buccae Cas13b (PbCas13b): K943, R1041, R1068, N1069, or H1073. In some cases, the Cas13 protein comprises in a HEPN domain one or more mutations of an amino acid corresponding to the following amino acids in a HEPN domain of Prevotella buccae Cas13b (PbCas13b): R53, Y164, K183, K193, K943, R1041, R56, N157, H161, R1068, N1069, or H1073. In some cases, the Cas13 protein comprises in HEPN domain 1 one or more mutations of an amino acid corresponding to the following amino acids in HEPN domain 1 of Prevotella buccae Cas13b (PbCas13b): R53, Y164, K183, K193, R56, N157, or H161.

In some cases, the Cas13 protein comprises in HEPN domain 2 one or more mutations of an amino acid corresponding to the following amino acids in HEPN domain 2 of Prevotella buccae Cas13b (PbCas13b): K943, R1041, R1068, N1069, or H1073. In some cases, the Cas13 protein comprises one or more mutations of an amino acid corresponding to the following amino acids of Prevotella buccae Cas13b (PbCas13b): K183 or K193. In some cases, the Cas13 protein comprises in HEPN domain 1 one or more mutations of an amino acid corresponding to the following amino acids in HEPN domain 1 of Prevotella buccae Cas13b (PbCas13b): K183 or K193.

In some cases, the Cas13 protein comprises one or more mutations of an amino acid corresponding to the following amino acids of Prevotella buccae Cas13b (PbCas13b): R53, Y164, K943, or R1041. In some cases, the Cas13 protein comprises in a HEPN domain one or more mutations of an amino acid corresponding to the following amino acids in a HEPN domain of Prevotella buccae Cas13b (PbCas13b): R53, Y164, K943, or R1041. In some cases, the Cas13 protein comprises one or more mutations of an amino acid corresponding to the following amino acids of Prevotella buccae Cas13b (PbCas13b): R53, K943, or R1041; preferably R53A, R53K, R53D, or R53E; K943A, K943R, K943D, or K943E; or R1041A, R1041K, R1041D, or R1041E.

In some cases, the Cas13 protein comprises in a HEPN domain one or more mutations of an amino acid corresponding to the following amino acids in a HEPN domain of Prevotella buccae Cas13b (PbCas13b): R53, K943, or R1041; preferably R53A, R53K, R53D, or R53E; K943A, K943R, K943D, or K943E; or R1041A, R1041K, R1041D, or R1041E.

In some cases, the Cas13 protein comprises a mutation of an amino acid corresponding to amino acid Y164 of Prevotella buccae Cas13b (PbCas13b), preferably Y164A, Y164F, or Y164W. In some cases, the Cas13 protein comprises HEPN domain 1 a mutations of an amino acid corresponding to amino acid Y164 HEPN domain 1 of Prevotella buccae Cas13b (PbCas13b), preferably Y164A, Y164F, or Y164W. In some cases, the Cas13 protein comprises one or more mutations of an amino acid corresponding to the following amino acids of Prevotella buccae Cas13b (PbCas13b): T405, H407, K457, D434, K431, R402, K393, R482, N480, D396, E397, D398, or E399.

In some cases, the Cas13 protein comprises in the LID domain one or more mutations of an amino acid corresponding to the following amino acids in the LID domain of Prevotella buccae Cas13b (PbCas13b): T405, H407, K457, D434, K431, R402, K393, R482, N480, D396, E397, D398, or E399. In some cases, the Cas13 protein comprises a mutation of an amino acid corresponding to amino acid H407 of Prevotella buccae Cas13b (PbCas13b), preferably H407Y, H407W, or H407F. In some cases, the Cas13 protein comprises one or more mutations of an amino acid corresponding to the following amino acids of Prevotella buccae Cas13b (PbCas13b): R402, K393, R482, N480, D396, E397, D398, or E399. In some cases, the Cas13 protein comprises in the LID domain one or more mutations of an amino acid corresponding to the following amino acids in the LID domain of Prevotella buccae Cas13b (PbCas13b): R402, K393, R482, N480, D396, E397, D398, or E399. In some cases, the Cas13 protein comprises one or more mutations of an amino acid corresponding to the following amino acids of Prevotella buccae Cas13b (PbCas13b): K457, D434, or K431. In some cases, the Cas13 protein comprises in the LID domain one or more mutations of an amino acid corresponding to the following amino acids in the LID domain of Prevotella buccae Cas13b (PbCas13b): K457, D434, or K431.

In some cases, the Cas13 protein comprises one or more mutations of an amino acid corresponding to the following amino acids of Prevotella buccae Cas13b (PbCas13b): H500, K570, K590, N634, R638, N652, N653, K655, S658, K741, K744, N756, S757, R762, R791, K846, K857, K870, R877, R600, K607, K612, R614, K617, K826, K828, K829, R824, R830, Q831, K835, K836, R838, R618, Q646, N647, N653, or N652. In some cases, the Cas13 protein comprises in a helical domain one or more mutations of an amino acid corresponding to the following amino acids in a helical domain of Prevotella buccae Cas13b (PbCas13b): H500, K570, K590, N634, R638, N652, N653, K655, S658, K741, K744, N756, S757, R762, R791, K846, K857, K870, R877, R600, K607, K612, R614, K617, K826, K828, K829, R824, R830, Q831, K835, K836, R838, R618, Q646, N647, N653, or N652. In some cases, the Cas13 protein comprises one or more mutations of an amino acid corresponding to the following amino acids of Prevotella buccae Cas13b (PbCas13b): H500, K570, N756, S757, R762, R791, K846, K857, K870, R877, K826, K828, K829, R824, R830, Q831, K835, K836, or R838. In some cases, the Cas13 protein comprises in helical domain 1 one or more mutations of an amino acid corresponding to the following amino acids in helical domain 1 of Prevotella buccae Cas13b (PbCas13b): H500, K570, N756, S757, R762, R791, K846, K857, K870, R877, K826, K828, K829, R824, R830, Q831, K835, K836, or R838.

In some cases, the Cas13 protein comprises one or more mutations of an amino acid corresponding to the following amino acids of Prevotella buccae Cas13b (PbCas13b): H500, K570, N756, S757, R762, or R791. In some cases, the Cas13 protein comprises in helical domain 1 one or more mutations of an amino acid corresponding to the following amino acids in helical domain 1 of Prevotella buccae Cas13b (PbCas13b): H500, K570, N756, S757, R762, or R791. In some cases, the Cas13 protein comprises one or more mutations of an amino acid corresponding to the following amino acids of Prevotella buccae Cas13b (PbCas13b): K846, K857, K870, R877, K826, K828, K829, R824, R830, Q831, K835, K836, or R838. In some cases, the Cas13 protein comprises in the helical bridge domain one or more mutations of an amino acid corresponding to the following amino acids in the helical bridge domain of Prevotella buccae Cas13b (PbCas13b): K846, K857, K870, R877, K826, K828, K829, R824, R830, Q831, K835, K836, or R838. In some cases, the Cas13 protein comprises one or more mutations of an amino acid corresponding to the following amino acids of Prevotella buccae Cas13b (PbCas13b): H500 or K570. In some cases, the Cas13 protein comprises in helical domain 1-2 one or more mutations of an amino acid corresponding to the following amino acids in helical domain 1-2 of Prevotella buccae Cas13b (PbCas13b): H500 or K570.

In some cases, the Cas13 protein comprises one or more mutations of an amino acid corresponding to the following amino acids of Prevotella buccae Cas13b (PbCas13b): N756, S757, R762, R791, K846, K857, K870, R877, K826, K828, K829, R824, R830, Q831, K835, K836, or R838. In some cases, the Cas13 protein comprises in helical domain 1-3 one or more mutations of an amino acid corresponding to the following amino acids in helical domain 1-3 of Prevotella buccae Cas13b (PbCas13b): N756, S757, R762, R791, K846, K857, K870, R877, K826, K828, K829, R824, R830, Q831, K835, K836, or R838. In some cases, the Cas13 protein comprises one or more mutations of an amino acid corresponding to the following amino acids of Prevotella buccae Cas13b (PbCas13b): N756, S757, R762, or R791. In some cases, the Cas13 protein comprises in helical domain 1-3 one or more mutations of an amino acid corresponding to the following amino acids in helical domain 1-3 of Prevotella buccae Cas13b (PbCas13b): N756, S757, R762, or R791. In some cases, the Cas13 protein comprises one or more mutations of an amino acid corresponding to the following amino acids of Prevotella buccae Cas13b (PbCas13b): N756, S757, R762, R791, K846, K857, K870, or R877. In some cases, the Cas13 protein comprises in helical domain 1-3 one or more mutations of an amino acid corresponding to the following amino acids in helical domain 1-3 of Prevotella buccae Cas13b (PbCas13b): N756, S757, R762, R791, K846, K857, K870, or R877.

In some cases, the Cas13 protein comprises one or more mutations of an amino acid corresponding to the following amino acids of Prevotella buccae Cas13b (PbCas13b): K826, K828, K829, R824, R830, Q831, K835, K836, or R838. In some cases, the Cas13 protein comprises in helical domain 1-3 one or more mutations of an amino acid corresponding to the following amino acids in helical domain 1-3 of Prevotella buccae Cas13b (PbCas13b): K826, K828, K829, R824, R830, Q831, K835, K836, or R838.

In some cases, the Cas13 protein comprises one or more mutations of an amino acid corresponding to the following amino acids of Prevotella buccae Cas13b (PbCas13b): K590, N634, R638, N652, N653, K655, S658, K741, K744, R600, K607, K612, R614, K617, R618, Q646, N647, N653, or N652. In some cases, the Cas13 protein comprises in helical domain 2 one or more mutations of an amino acid corresponding to the following amino acids in helical domain 2 of Prevotella buccae Cas13b (PbCas13b): K590, N634, R638, N652, N653, K655, S658, K741, K744, R600, K607, K612, R614, K617, R618, Q646, N647, N653, or N652. In some cases, the Cas13 protein comprises one or more mutations of an amino acid corresponding to the following amino acids of Prevotella buccae Cas13b (PbCas13b): Q646 or N647. In some cases, the Cas13 protein comprises in helical domain 2 one or more mutations of an amino acid corresponding to the following amino acids in helical domain 2 of Prevotella buccae Cas13b (PbCas13b): Q646 or N647. In some cases, the Cas13 protein comprises one or more mutations of an amino acid corresponding to the following amino acids of Prevotella buccae Cas13b (PbCas13b): N653 or N652. In some cases, the Cas13 protein comprises in helical domain 2 one or more mutations of an amino acid corresponding to the following amino acids in helical domain 2 of Prevotella buccae Cas13b (PbCas13b): N653 or N652. In some cases, the Cas13 protein comprises one or more mutations of an amino acid corresponding to the following amino acids of Prevotella buccae Cas13b (PbCas13b): K590, N634, R638, N652, N653, K655, S658, K741, or K744. In some cases, the Cas13 protein comprises in helical domain 2 one or more mutations of an amino acid corresponding to the following amino acids in helical domain 2 of Prevotella buccae Cas13b (PbCas13b): K590, N634, R638, N652, N653, K655, S658, K741, or K744. In some cases, the Cas13 protein comprises one or more mutations of an amino acid corresponding to the following amino acids of Prevotella buccae Cas13b (PbCas13b): R600, K607, K612, R614, K617, or R618. In some cases, the Cas13 protein comprises in helical domain 2 one or more mutations of an amino acid corresponding to the following amino acids in helical domain 2 of Prevotella buccae Cas13b (PbCas13b): R600, K607, K612, R614, K617, or R618. In some cases, the Cas13 protein comprises one or more mutations of an amino acid corresponding to the following amino acids of Prevotella buccae Cas13b (PbCas13b): R285, R287, K292, E296, N297, or K294. In some cases, the Cas13 protein comprises in the IDL domain one or more mutations of an amino acid corresponding to the following amino acids in the IDL domain of Prevotella buccae Cas13b (PbCas13b): R285, R287, K292, E296, N297, or K294. In some cases, the Cas13 protein comprises one or more mutations of an amino acid corresponding to the following amino acids of Prevotella buccae Cas13b (PbCas13b): R285, K292, E296, or N297. In some cases, the Cas13 protein comprises in the IDL domain one or more mutations of an amino acid corresponding to the following amino acids in the IDL domain of Prevotella buccae Cas13b (PbCas13b): R285, K292, E296, or N297.

In some cases, the Cas13 protein comprises one or more mutations of an amino acid corresponding to the following amino acids of Prevotella buccae Cas13b (PbCas13b): T405, H500, K570, K590, N634, R638, N652, N653, K655, S658, K741, K744, N756, S757, R762, R791, K846, K857, K870, R877, K183, K193, R600, K607, K612, R614, K617, K826, K828, K829, R824, R830, Q831, K835, K836, R838, R618, D434, K431, R285, R287, K292, E296, N297, Q646, N647, or K294. In some cases, the Cas13 protein comprises one or more mutations of an amino acid corresponding to the following amino acids of Prevotella buccae Cas13b (PbCas13b): R402, K393, N653, N652, R482, N480, D396, E397, D398, or E399. In some cases, the Cas13 protein comprises one or more mutations of an amino acid corresponding to the following amino acids of Prevotella buccae Cas13b (PbCas13b): R53, K655, R762, or R1041; preferably R53A or R53D; K655A; R762A; or R1041E or R1041D. In some cases, the Cas13 protein comprises one or more mutations of an amino acid corresponding to the following amino acids of Prevotella buccae Cas13b (PbCas13b): N297, E296, K292, or R285; preferably N297A, E296A, K292A, or R285A. In some cases, the Cas13 protein comprises in (e.g., the central channel of) the IDL domain one or more mutations of an amino acid corresponding to the following amino acids in (e.g., the central channel of) the IDL domain of Prevotella buccae Cas13b (PbCas13b): N297, E296, K292, or R285; preferably N297A, E296A, K292A, or R285A. In some cases, the Cas13 protein comprises one or more mutations of an amino acid corresponding to the following amino acids of Prevotella buccae Cas13b (PbCas13b): Q831, K836, R838, N652, N653, R830, K655 or R762; preferably Q831A, K836A, R838A, N652A, N653A, R830A, K655A, or R762A.

In some cases, the Cas13 protein comprises one or more mutations of an amino acid corresponding to the following amino acids of Prevotella buccae Cas13b (PbCas13b): N652, N653, R830, K655 or R762; preferably N652A, N653A, R830A, K655A, or R762A. In some cases, the Cas13 protein comprises one or more mutations of an amino acid corresponding to the following amino acids of Prevotella buccae Cas13b (PbCas13b): K655 or R762; preferably K655A or R762A. In some cases, the Cas13 protein comprises in a helical domain one or more mutations of an amino acid corresponding to the following amino acids in a helical domain of Prevotella buccae Cas13b (PbCas13b): Q831, K836, R838, N652, N653, R830, K655 or R762; preferably Q831A, K836A, R838A, N652A, N653A, R830A, K655A, or R762A. In some cases, the Cas13 protein comprises a helical domain one or more mutations of an amino acid corresponding to the following amino acids a helical domain of Prevotella buccae Cas13b (PbCas13b): N652, N653, R830, K655 or R762; preferably N652A, N653A, R830A, K655A, or R762A.

In some cases, the Cas13 protein comprises in helical domain 2 one or more mutations of an amino acid corresponding to the following amino acids in helical domain 2 of Prevotella buccae Cas13b (PbCas13b): K655 or R762; preferably K655A or R762A. In some cases, the Cas13 protein comprises one or more mutations of an amino acid corresponding to the following amino acids of Prevotella buccae Cas13b (PbCas13b): R614, K607, K193, K183 or R600; preferably R614A, K607A, K193A, K183A or R600A. In some cases, the Cas13 protein comprises in the trans-subunit loop of helical domain 2 one or more mutations of an amino acid corresponding to the following amino acids in the trans-subunit loop of helical domain 2 of Prevotella buccae Cas13b (PbCas13b): Q646 or N647; preferably Q646A or N647A. In some cases, the Cas13 protein comprises one or more mutations of an amino acid corresponding to the following amino acids of Prevotella buccae Cas13b (PbCas13b): R53 or R1041; preferably R53A or R53D, or R1041E or R1041D. In some cases, the Cas13 protein comprises in a HEPN domain one or more mutations of an amino acid corresponding to the following amino acids in a HEPN domain of Prevotella buccae Cas13b (PbCas13b): R53 or R1041; preferably R53A or R53D, or R1041E or R1041D. In some cases, the Cas13 protein comprises one or more mutations of an amino acid corresponding to the following amino acids of Prevotella buccae Cas13b (PbCas13b): K457, D397, E398, D399, E400, T405, H407 or D434; preferably D397A, E398A, D399A, E400A, T405A, H407A, H407W, H407Y, H407F or D434A. In some cases, the Cas13 protein comprises in the LID domain one or more mutations of an amino acid corresponding to the following amino acids in the LID domain of Prevotella buccae Cas13b (PbCas13b): K457, D397, E398, D399, E400, T405, H407 or D434; preferably D397A, E398A, D399A, E400A, T405A, H407A, H407W, H407Y, H407F or D434A.

In some cases, the Cas13 protein comprises a mutation of an amino acid corresponding to amino acid T405 of Prevotella buccae Cas13b (PbCas13b). In some cases, the Cas13 protein comprises a mutation of an amino acid corresponding to amino acid H407 of Prevotella buccae Cas13b (PbCas13b). In some cases, the Cas13 protein comprises a mutation of an amino acid corresponding to amino acid K457 of Prevotella buccae Cas13b (PbCas13b). In some cases, the Cas13 protein comprises a mutation of an amino acid corresponding to amino acid H500 of Prevotella buccae Cas13b (PbCas13b). In some cases, the Cas13 protein comprises a mutation of an amino acid corresponding to amino acid K570 of Prevotella buccae Cas13b (PbCas13b). In some cases, the Cas13 protein comprises a mutation of an amino acid corresponding to amino acid K590 of Prevotella buccae Cas13b (PbCas13b). In some cases, the Cas13 protein comprises a mutation of an amino acid corresponding to amino acid N634 of Prevotella buccae Cas13b (PbCas13b). In some cases, the Cas13 protein comprises a mutation of an amino acid corresponding to amino acid R638 of Prevotella buccae Cas13b (PbCas13b). In some cases, the Cas13 protein comprises a mutation of an amino acid corresponding to amino acid N652 of Prevotella buccae Cas13b (PbCas13b). In some cases, the Cas13 protein comprises a mutation of an amino acid corresponding to amino acid N653 of Prevotella buccae Cas13b (PbCas13b). In some cases, the Cas13 protein comprises a mutation of an amino acid corresponding to amino acid K655 of Prevotella buccae Cas13b (PbCas13b). In some cases, the Cas13 protein comprises a mutation of an amino acid corresponding to amino acid S658 of Prevotella buccae Cas13b (PbCas13b). In some cases, the Cas13 protein comprises a mutation of an amino acid corresponding to amino acid K741 of Prevotella buccae Cas13b (PbCas13b). In some cases, the Cas13 protein comprises a mutation of an amino acid corresponding to amino acid K744 of Prevotella buccae Cas13b (PbCas13b). In some cases, the Cas13 protein comprises a mutation of an amino acid corresponding to amino acid N756 of Prevotella buccae Cas13b (PbCas13b). In some cases, the Cas13 protein comprises a mutation of an amino acid corresponding to amino acid S757 of Prevotella buccae Cas13b (PbCas13b). In some cases, the Cas13 protein comprises a mutation of an amino acid corresponding to amino acid R762 of Prevotella buccae Cas13b (PbCas13b). In some cases, the Cas13 protein comprises a mutation of an amino acid corresponding to amino acid R791 of Prevotella buccae Cas13b (PbCas13b). In some cases, the Cas13 protein comprises a mutation of an amino acid corresponding to amino acid K846 of Prevotella buccae Cas13b (PbCas13b). In some cases, the Cas13 protein comprises a mutation of an amino acid corresponding to amino acid K857 of Prevotella buccae Cas13b (PbCas13b). In some cases, the Cas13 protein comprises a mutation of an amino acid corresponding to amino acid K870 of Prevotella buccae Cas13b (PbCas13b). In some cases, the Cas13 protein comprises a mutation of an amino acid corresponding to amino acid R877 of Prevotella buccae Cas13b (PbCas13b). In some cases, the Cas13 protein comprises a mutation of an amino acid corresponding to amino acid K183 of Prevotella buccae Cas13b (PbCas13b). In some cases, the Cas13 protein comprises a mutation of an amino acid corresponding to amino acid K193 of Prevotella buccae Cas13b (PbCas13b). In some cases, the Cas13 protein comprises a mutation of an amino acid corresponding to amino acid R600 of Prevotella buccae Cas13b (PbCas13b). In some cases, the Cas13 protein comprises a mutation of an amino acid corresponding to amino acid K607 of Prevotella buccae Cas13b (PbCas13b). In some cases, the Cas13 protein comprises a mutation of an amino acid corresponding to amino acid K612 of Prevotella buccae Cas13b (PbCas13b). In some cases, the Cas13 protein comprises a mutation of an amino acid corresponding to amino acid R614 of Prevotella buccae Cas13b (PbCas13b). In some cases, the Cas13 protein comprises a mutation of an amino acid corresponding to amino acid K617 of Prevotella buccae Cas13b (PbCas13b). In some cases, the Cas13 protein comprises a mutation of an amino acid corresponding to amino acid K826 of Prevotella buccae Cas13b (PbCas13b). In some cases, the Cas13 protein comprises a mutation of an amino acid corresponding to amino acid K828 of Prevotella buccae Cas13b (PbCas13b). In some cases, the Cas13 protein comprises a mutation of an amino acid corresponding to amino acid K829 of Prevotella buccae Cas13b (PbCas13b). In some cases, the Cas13 protein comprises a mutation of an amino acid corresponding to amino acid R824 of Prevotella buccae Cas13b (PbCas13b). In some cases, the Cas13 protein comprises a mutation of an amino acid corresponding to amino acid R830 of Prevotella buccae Cas13b (PbCas13b). In some cases, the Cas13 protein comprises a mutation of an amino acid corresponding to amino acid Q831 of Prevotella buccae Cas13b (PbCas13b).

In some cases, the Cas13 protein comprises a mutation of an amino acid corresponding to amino acid K835 of Prevotella buccae Cas13b (PbCas13b). In some cases, the Cas13 protein comprises a mutation of an amino acid corresponding to amino acid K836 of Prevotella buccae Cas13b (PbCas13b). In some cases, the Cas13 protein comprises a mutation of an amino acid corresponding to amino acid R838 of Prevotella buccae Cas13b (PbCas13b). In some cases, the Cas13 protein comprises a mutation of an amino acid corresponding to amino acid R618 of Prevotella buccae Cas13b (PbCas13b). In some cases, the Cas13 protein comprises a mutation of an amino acid corresponding to amino acid D434 of Prevotella buccae Cas13b (PbCas13b). In some cases, the Cas13 protein comprises a mutation of an amino acid corresponding to amino acid K431 of Prevotella buccae Cas13b (PbCas13b). In some cases, the Cas13 protein comprises a mutation of an amino acid corresponding to amino acid R53 of Prevotella buccae Cas13b (PbCas13b). In some cases, the Cas13 protein comprises a mutation of an amino acid corresponding to amino acid K943 of Prevotella buccae Cas13b (PbCas13b). In some cases, the Cas13 protein comprises a mutation of an amino acid corresponding to amino acid R1041 of Prevotella buccae Cas13b (PbCas13b). In some cases, the Cas13 protein comprises a mutation of an amino acid corresponding to amino acid Y164 of Prevotella buccae Cas13b (PbCas13b). In some cases, the Cas13 protein comprises a mutation of an amino acid corresponding to amino acid R285 of Prevotella buccae Cas13b (PbCas13b). In some cases, the Cas13 protein comprises a mutation of an amino acid corresponding to amino acid R287 of Prevotella buccae Cas13b (PbCas13b). In some cases, the Cas13 protein comprises a mutation of an amino acid corresponding to amino acid K292 of Prevotella buccae Cas13b (PbCas13b). In some cases, the Cas13 protein comprises a mutation of an amino acid corresponding to amino acid E296 of Prevotella buccae Cas13b (PbCas13b). In some cases, the Cas13 protein comprises a mutation of an amino acid corresponding to amino acid N297 of Prevotella buccae Cas13b (PbCas13b). In some cases, the Cas13 protein comprises a mutation of an amino acid corresponding to amino acid Q646 of Prevotella buccae Cas13b (PbCas13b).

In some cases, the Cas13 protein comprises a mutation of an amino acid corresponding to amino acid N647 of Prevotella buccae Cas13b (PbCas13b). In some cases, the Cas13 protein comprises a mutation of an amino acid corresponding to amino acid R402 of Prevotella buccae Cas13b (PbCas13b). In some cases, the Cas13 protein comprises a mutation of an amino acid corresponding to amino acid K393 of Prevotella buccae Cas13b (PbCas13b). In some cases, the Cas13 protein comprises a mutation of an amino acid corresponding to amino acid N653 of Prevotella buccae Cas13b (PbCas13b). In some cases, the Cas13 protein comprises a mutation of an amino acid corresponding to amino acid N652 of Prevotella buccae Cas13b (PbCas13b). In some cases, the Cas13 protein comprises a mutation of an amino acid corresponding to amino acid R482 of Prevotella buccae Cas13b (PbCas13b). In some cases, the Cas13 protein comprises a mutation of an amino acid corresponding to amino acid N480 of Prevotella buccae Cas13b (PbCas13b). In some cases, the Cas13 protein comprises a mutation of an amino acid corresponding to amino acid D396 of Prevotella buccae Cas13b (PbCas13b). In some cases, the Cas13 protein comprises a mutation of an amino acid corresponding to amino acid E397 of Prevotella buccae Cas13b (PbCas13b). In some cases, the Cas13 protein comprises a mutation of an amino acid corresponding to amino acid D398 of Prevotella buccae Cas13b (PbCas13b). In some cases, the Cas13 protein comprises a mutation of an amino acid corresponding to amino acid E399 of Prevotella buccae Cas13b (PbCas13b). In some cases, the Cas13 protein comprises a mutation of an amino acid corresponding to amino acid K294 of Prevotella buccae Cas13b (PbCas13b). In some cases, the Cas13 protein comprises a mutation of an amino acid corresponding to amino acid E400 of Prevotella buccae Cas13b (PbCas13b). In some cases, the Cas13 protein comprises a mutation of an amino acid corresponding to amino acid R56 of Prevotella buccae Cas13b (PbCas13b). In some cases, the Cas13 protein comprises a mutation of an amino acid corresponding to amino acid N157 of Prevotella buccae Cas13b (PbCas13b). In some cases, the Cas13 protein comprises a mutation of an amino acid corresponding to amino acid H161 of Prevotella buccae Cas13b (PbCas13b). In some cases, the Cas13 protein comprises a mutation of an amino acid corresponding to amino acid H452 of Prevotella buccae Cas13b (PbCas13b). In some cases, the Cas13 protein comprises a mutation of an amino acid corresponding to amino acid N455 of Prevotella buccae Cas13b (PbCas13b). In some cases, the Cas13 protein comprises a mutation of an amino acid corresponding to amino acid K484 of Prevotella buccae Cas13b (PbCas13b). In some cases, the Cas13 protein comprises a mutation of an amino acid corresponding to amino acid N486 of Prevotella buccae Cas13b (PbCas13b). In some cases, the Cas13 protein comprises a mutation of an amino acid corresponding to amino acid G566 of Prevotella buccae Cas13b (PbCas13b). In some cases, the Cas13 protein comprises a mutation of an amino acid corresponding to amino acid H567 of Prevotella buccae Cas13b (PbCas13b). In some cases, the Cas13 protein comprises a mutation of an amino acid corresponding to amino acid A656 of Prevotella buccae Cas13b (PbCas13b). In some cases, the Cas13 protein comprises a mutation of an amino acid corresponding to amino acid V795 of Prevotella buccae Cas13b (PbCas13b). In some cases, the Cas13 protein comprises a mutation of an amino acid corresponding to amino acid A796 of Prevotella buccae Cas13b (PbCas13b). In some cases, the Cas13 protein comprises a mutation of an amino acid corresponding to amino acid W842 of Prevotella buccae Cas13b (PbCas13b). In some cases, the Cas13 protein comprises a mutation of an amino acid corresponding to amino acid K871 of Prevotella buccae Cas13b (PbCas13b). In some cases, the Cas13 protein comprises a mutation of an amino acid corresponding to amino acid E873 of Prevotella buccae Cas13b (PbCas13b). In some cases, the Cas13 protein comprises a mutation of an amino acid corresponding to amino acid R874 of Prevotella buccae Cas13b (PbCas13b). In some cases, the Cas13 protein comprises a mutation of an amino acid corresponding to amino acid R1068 of Prevotella buccae Cas13b (PbCas13b). In some cases, the Cas13 protein comprises a mutation of an amino acid corresponding to amino acid N1069 of Prevotella buccae Cas13b (PbCas13b). In some cases, the Cas13 protein comprises a mutation of an amino acid corresponding to amino acid H1073 of Prevotella buccae Cas13b (PbCas13b).

In some cases, the Cas13 protein comprises one or more mutations of an amino acid corresponding to the following amino acids of Leptotrichia shahii Cas 13a (LshCas13a): R597, N598, H602, R1278, N1279, or H1283. The present disclosure also includes a mutated Cas13 protein comprising one or more mutations of an amino acid corresponding to the following amino acids of Leptotrichia shahii Cas 13a (LshCas13a): R597, N598, H602, R1278, N1279, or H1283. In some cases, the Cas13 protein comprises in a HEPN domain one or more mutations of an amino acid corresponding to the following amino acids in a HEPN domain of Leptotrichia shahii Cas13a (LshCas13a): R597, N598, H602, R1278, N1279, or H1283. In some cases, the Cas13 protein comprises one or more mutations of an amino acid corresponding to the following amino acids of Leptotrichia shahii Cas 13a (LshCas13a): R597, N598, or H602. In some cases, the Cas13 protein comprises in HEPN domain 1 one or more mutation of an amino acid corresponding to the following amino acids in HEPN domain 1 of Leptotrichia shahii Cas13a (LshCas13a): R597, N598, or H602. In some cases, the Cas13 protein comprises one or more mutations of an amino acid corresponding to the following amino acids of Leptotrichia shahii Cas 13a (LshCas13a): R1278, N1279, or H1283. In some cases, the Cas13 protein comprises in HEPN domain 2 one or more mutations of an amino acid corresponding to the following amino acids in HEPN domain 2 of Leptotrichia shahii Cas 13a (LshCas13a): R1278, N1279, or H1283. In some cases, the Cas13 protein comprises one or more mutations of an amino acid corresponding to the following amino acids of Porphyromonas gulae Cas13b (PguCas13b): R146, H151, R1116, or H1121. In some cases, the Cas13 protein comprises one or more mutations of an amino acid corresponding to the following amino acids of Porphyromonas gulae Cas13b (PguCas13b): R146, H151, R1116, or H1121. In some cases, the Cas13 protein comprises in a HEPN domain one or more mutations of an amino acid corresponding to the following amino acids in a HEPN domain of Porphyromonas gulae Cas13b (PguCas13b): R146, H151, R1116, or H1121.

In some cases, the Cas13 protein comprises one or more mutations of an amino acid corresponding to the following amino acids of Porphyromonas gulae Cas13b (PguCas13b): R146 or H151. In some cases, the Cas13 protein comprises in HEPN domain 1 one or more mutations of an amino acid corresponding to the following amino acids in HEPN domain 1 of Porphyromonas gulae Cas13b (PguCas13b): R146 or H151. In some cases, the Cas13 protein comprises one or more mutations of an amino acid corresponding to the following amino acids of Porphyromonas gulae Cas13b (PguCas13b): R1116 or H1121. In some cases, the Cas13 protein comprises in HEPN domain 2 one or more mutations of an amino acid corresponding to the following amino acids in HEPN domain 2 of Porphyromonas gulae Cas13b (PguCas13b): R1116 or H1121. In some cases, the Cas13 protein comprises one or more mutations of an amino acid corresponding to the following amino acids of Prevotella sp. P5-125 Cas13b (PspCas13b): H133 or H1058. The present disclosure also provides a mutated Cas13 protein comprising one or more mutations of an amino acid corresponding to the following amino acids of Prevotella sp. P5-125 Cas13b (PspCas13b): H133 or H1058. In some cases, the Cas13 protein comprises in a HEPN domain one or more mutations of an amino acid corresponding to the following amino acids in a HEPN domain of Prevotella sp. P5-125 Cas13b (PspCas13b): H133 or H1058.

In some cases, the Cas13 protein comprises a mutation of an amino acid corresponding to amino acid H133 of Prevotella sp. P5-125 Cas13b (PspCas13b). In some cases, the Cas13 protein comprises in HEPN domain 1 a mutation of an amino acid corresponding to amino acid H133 in HEPN domain 1 of Prevotella sp. P5-125 Cas13b (PspCas13b). In some cases, the Cas13 protein comprises a mutation of an amino acid corresponding to amino acid H1058 of Prevotella sp. P5-125 Cas13b (PspCas13b). In some cases, the Cas13 protein comprises in HEPN domain 2 a mutation of an amino acid corresponding to the amino acid H1058 in HEPN domain 2 of Prevotella sp. P5-125 Cas13b (PspCas13b).

The Cas protein herein may comprise one or more amino acids mutated. In some embodiments, the amino acid is mutated to A, P, or V, preferably A. In some embodiments, the amino acid is mutated to a hydrophobic amino acid. In some embodiments, the amino acid is mutated to an aromatic amino acid. In some embodiments, the amino acid is mutated to a charged amino acid. In some embodiments, the amino acid is mutated to a positively charged amino acid. In some embodiments, the amino acid is mutated to a negatively charged amino acid. In some embodiments, the amino acid is mutated to a polar amino acid. In some embodiments, the amino acid is mutated to an aliphatic amino acid.

### Structural (Sub)Domains

In another aspect, the disclosure provides a mutated Cas13 protein comprising one or more mutations of amino acids, wherein the amino acids: interact with a guide RNA that forms a complex with the engineered Cas 13 protein; or are in a HEPN active site, a lid domain, a helical domain, selected from a helical 1 or a helical 2 domain, an inter-domain linker (IDL) domain, or a bridge helix domain of the mutated Cas 13 protein, or a combination thereof.

Based on the crystal structure of the Cas protein, different structural domains can be identified. In addition to sequence alignments, the information of the crystal structure and domain architecture allows corresponding amino acids of different orthologs (e.g. Cas13b orthologs) and homologs (other Cas13 proteins, such as Cas13a, Cas13c, or Cas13d) to be identified. By means of example, and without limitation, the crystal structure of PbCas13b in complex with crRNA as reported herein, identifies the following structural domains: HEPN1 and HEPN2 (catalytic domains, respectively spanning from amino acid 1 to 285 and 930 to 1127); IDL (interdomain linker, spanning from amino acids 286 to 301); helical domains 1 and 2, whereby helical domain is split in helical domain 1-1, 1-2, and 1-3 (respectively spanning from amino acids 302 to 374, 499 to 581, and 747 to 929), and helical domain 2 spanning from amino acids 582 to 746; LID (spanning from amino acids 375 to 498). Helical domain 1, in particular helical domain 1-3 encompasses a bridge helix as a discernible subdomain. Accordingly, particular mutations according to the invention as described herein, apart from having a specified amino acid position in the Cas13 polypeptide can also be linked to a particular structural domain of the Cas13 protein. Hence a corresponding amino acid in a Cas13 ortholog or homolog can have a specified amino acid position in the Cas13 polypeptide as well as belong to a corresponding structural domain. Mutations may be identified by locations in structural (sub) domains, by position corresponding to amino acids of a particular Cas13 protein (e.g. PbCas13b), by interactions with a guide RNA, or a combination thereof.

The types of mutations can be conservative mutations or non-conservative mutations. In certain preferred embodiments, the amino acid which is mutated is mutated into alanine (A). In certain preferred embodiments, if the amino acid to be mutated is an aromatic amino acid, it is mutated into alanine or another aromatic amino acid (e.g. H, Y, W, or F). In certain preferred embodiments, if the amino acid to be mutated is a charged amino acid, it is mutated into alanine or another charged amino acid (e.g. H, K, R, D, or E). In certain preferred embodiments, if the amino acid to be mutated is a charged amino acid, it is mutated into alanine or another charged amino acid having the same charge. In certain preferred embodiments, if the amino acid to be mutated is a charged amino acid, it is mutated into alanine or another charged amino acid having the opposite charge.

In some embodiments, the invention also provides for methods and compositions wherein one or more amino acid residues of the effector protein may be modified e.g., an engineered or non-naturally-occurring effector protein or Cas13. In an embodiment, the modification may comprise mutation of one or more amino acid residues of the effector protein. The one or more mutations may be in one or more catalytically active domains of the effector protein, or a domain interacting with the crRNA (such as the guide sequence or direct repeat sequence). The effector protein may have reduced or abolished nuclease activity or alternatively increased nuclease activity compared with an effector protein lacking said one or more mutations. The effector protein may not direct cleavage of the RNA strand at the target locus of interest. In a preferred embodiment, the one or more mutations may comprise two mutations. In a preferred embodiment the one or more amino acid residues are modified in a Cas13 protein, e.g., an engineered or non-naturally-occurring effector protein or Cas13. In some cases, the CRISPR-Cas protein comprises one or more mutations in the helical domain.

The present disclosure also provides for methods of altering activity of CRISPR-Cas proteins. In some examples, such methods comprise identifying one or more candidate amino acids in the Cas13 protein based on a three-dimensional structure of at least a portion of the Cas 13 protein, wherein the one or more candidate amino acids interact with a guide RNA that forms a complex with the Cas13 protein, or are in a HEPN active site, an inter-domain linker domain, or a bridge helix domain of the Cas13 protein; and mutating the one or more candidate amino acids thereby generating a mutated Cas13 protein, wherein activity the mutated Cas13 protein is different than the Cas13 protein.

### Destabilized Cas13 and Fusion Proteins

In certain embodiments, the Cas protein according to the invention as described herein is associated with or fused to a destabilization domain (DD). In some embodiments, the DD is ER50. A corresponding stabilizing ligand for this DD is, in some embodiments, 4HT. As such, in some embodiments, one of the at least one DDs is ER50 and a stabilizing ligand therefor is 4HT or CMP8. In some embodiments, the DD is DHFR50. A corresponding stabilizing ligand for this DD is, in some embodiments, TMP. As such, in some embodiments, one of the at least one DDs is DHFR50 and a stabilizing ligand therefor is TMP. In some embodiments, the DD is ER50. A corresponding stabilizing ligand for this DD is, in some embodiments, CMP8. CMP8 may therefore be an alternative stabilizing ligand to 4HT in the ER50 system. While it may be possible that CMP8 and 4HT can/should be used in a competitive matter, some cell types may be more susceptible to one or the other of these two ligands, and from this disclosure and the knowledge in the art the skilled person can use CMP8 and/or 4HT.

In some embodiments, one or two DDs may be fused to the N-terminal end of the Cas with one or two DDs fused to the C-terminal of the Cas. In some embodiments, the at least two DDs are associated with the Cas13 and the DDs are the same DD, i.e. the DDs are homologous. Thus, both (or two or more) of the DDs could be ER50 DDs. This is preferred in some embodiments. Alternatively, both (or two or more) of the DDs could be DHFR50 DDs. This is also preferred in some embodiments. In some embodiments, the at least two DDs are associated with the Cas and the DDs are different DDs, i.e. the DDs are heterologous. Thus, one of the DDS could be ER50 while one or more of the DDs or any other DDs could be DHFR50. Having two or more DDs which are heterologous may be advantageous as it would provide a greater level of degradation control. A tandem fusion of more than one DD at the N or C-term may enhance degradation; and such a tandem fusion can be, for example ER50-ER50-Cas or DHFR-DHFR-Cas It is envisaged that high levels of degradation would occur in the absence of either stabilizing ligand, intermediate levels of degradation would occur in the absence of one stabilizing ligand and the presence of the other (or another) stabilizing ligand, while low levels of degradation would occur in the presence of both (or two of more) of the stabilizing ligands. Control may also be imparted by having an N-terminal ER50 DD and a C-terminal DHFR50 DD.

In some embodiments, the fusion of the Cas with the DD comprises a linker between the DD and the Cas13. In some embodiments, the linker is a GlySer linker. In some embodiments, the DD-Cas13 further comprises at least one Nuclear Export Signal (NES). In some embodiments, the DD-Cas13 comprises two or more NESs. In some embodiments, the DD-Cas comprises at least one Nuclear Localization Signal (NLS). This may be in addition to an NES. In some embodiments, the Cas13 comprises or consists essentially of or consists of a localization (nuclear import or export) signal as, or as part of, the linker between the Cas13 and the DD. HA or Flag tags are also within the ambit of the invention as linkers. Applicants use NLS and/or NES as linker and also use Glycine Serine linkers as short as GS up to (GGGGS)₃ (SEQ ID NO: 5204).

Destabilizing domains have general utility to confer instability to a wide range of proteins; see, e.g., Miyazaki, J Am Chem Soc. Mar. 7, 2012; 134(9): 3942-3945, incorporated herein by reference. CMP8 or 4-hydroxytamoxifen can be destabilizing domains. More generally, A temperature-sensitive mutant of mammalian DHFR (DHFRts), a destabilizing residue by the N-end rule, was found to be stable at a permissive temperature but unstable at 37° C. The addition of methotrexate, a high-affinity ligand for mammalian DHFR, to cells expressing DHFRts inhibited degradation of the protein partially. This was an important demonstration that a small molecule ligand can stabilize a protein otherwise targeted for degradation in cells. A rapamycin derivative was used to stabilize an unstable mutant of the FRB domain of mTOR (FRB*) and restore the function of the fused kinase, GSK-3β.6,7 This system demonstrated that ligand-dependent stability represented an attractive strategy to regulate the function of a specific protein in a complex biological environment. A system to control protein activity can involve the DD becoming functional when the ubiquitin complementation occurs by rapamycin induced dimerization of FK506-binding protein and FKBP12. Mutants of human FKBP12 or ecDHFR protein can be engineered to be metabolically unstable in the absence of their high-affinity ligands, Shield-1 or trimethoprim (TMP), respectively. These mutants are some of the possible destabilizing domains (DDs) useful in the practice of the invention and instability of a DD as a fusion with a Cas13 confers to the Cas13 degradation of the entire fusion protein by the proteasome. Shield-1 and TMP bind to and stabilize the DD in a dose-dependent manner. The estrogen receptor ligand binding domain (ERLBD, residues 305-549 of ERS1) can also be engineered as a destabilizing domain. Since the estrogen receptor signaling pathway is involved in a variety of diseases such as breast cancer, the pathway has been widely studied and numerous agonist and antagonists of estrogen receptor have been developed. Thus, compatible pairs of ERLBD and drugs are known. There are ligands that bind to mutant but not wild-type forms of the ERLBD. By using one of these mutant domains encoding three mutations (L384M, M421G, G521R)12, it is possible to regulate the stability of an ERLBD-derived DD using a ligand that does not perturb endogenous estrogen-sensitive networks. An additional mutation (Y537S) can be introduced to further destabilize the ERLBD and to configure it as a potential DD candidate. This tetra-mutant is an advantageous DD development. The mutant ERLBD can be fused to a Cas13 and its stability can be regulated or perturbed using a ligand, whereby the Cas13 has a DD. Another DD can be a 12-kDa (107-amino-acid) tag based on a mutated FKBP protein, stabilized by Shield1 ligand; see, e.g., Nature Methods 5, (2008). For instance a DD can be a modified FK506 binding protein 12 (FKBP12) that binds to and is reversibly stabilized by a synthetic, biologically inert small molecule, Shield-1; see, e.g., Banaszynski L. A., Chen L. C., Maynard-Smith L. A., Ooi A. G., Wandless T. J. A rapid, reversible, and tunable method to regulate protein function in living cells using synthetic small molecules. Cell. 2006; 126:995-1004; Banaszynski L. A., Sellmyer M. A., Contag C. H., Wandless T. J., Thorne S. H. Chemical control of protein stability and function in living mice. Nat Med. 2008; 14:1123-1127; Maynard-Smith L. A., Chen L. C., Banaszynski L. A., Ooi A. G., Wandless T. J. A directed approach for engineering conditional protein stability using biologically silent small molecules. The Journal of biological chemistry. 2007; 282:24866-24872; and Rodriguez, Chem Biol. Mar. 23, 2012; 19(3): 391-398-all of which are incorporated herein by reference and may be employed in the practice of the invention in selected a DD to associate with a Cas13 in the practice of this invention. As can be seen, the knowledge in the art includes a number of DDs, and the DD can be associated with, e.g., fused to, advantageously with a linker, to a Cas13, whereby the DD can be stabilized in the presence of a ligand and when there is the absence thereof the DD can become destabilized, whereby the Cas13 is entirely destabilized, or the DD can be stabilized in the absence of a ligand and when the ligand is present the DD can become destabilized; the DD allows the Cas13 and hence the CRISPR-Cas13 complex or system to be regulated or controlled-turned on or of f so to speak, to thereby provide means for regulation or control of the system, e.g., in an in vivo or in vitro environment. For instance, when a protein of interest is expressed as a fusion with the DD tag, it is destabilized and rapidly degraded in the cell, e.g., by proteasomes. Thus, absence of stabilizing ligand leads to a D associated Cas being degraded. When a new DD is fused to a protein of interest, its instability is conferred to the protein of interest, resulting in the rapid degradation of the entire fusion protein. Peak activity for Cas is sometimes beneficial to reduce off-target effects. Thus, short bursts of high activity are preferred. The present invention is able to provide such peaks. In some senses the system is inducible. In some other senses, the system repressed in the absence of stabilizing ligand and de-repressed in the presence of stabilizing ligand.

### Dead Cas Proteins

In certain embodiments, the Cas protein herein is a catalytically inactive or dead Cas protein. In some cases, Cas protein herein is a catalytically inactive or dead Cas13 effector protein (dCas13). In some cases, a dead Cas protein, e.g., a dead Cas13 protein has nickase activity. In some embodiments, the dCas13 protein comprises mutations in the nuclease domain. In some embodiments, the dCas13 effector protein has been truncated. In some cases, the dead Cas proteins may be fused with a deaminase herein, e.g., an adenosine deaminase.

To reduce the size of a fusion protein of the Cas13 protein and the one or more functional domains, the C-terminus of the Cas13 protein can be truncated while still maintaining its RNA binding function. For example, at least 20 amino acids, at least 40 amino acids, at least 50 amino acids, at least 60 amino acids, at least 80 amino acids, at least 100 amino acids, at least 120 amino acids, at least 140 amino acids, at least 150 amino acids, at least 160 amino acids, at least 180 amino acids, at least 200 amino acids, at least 220 amino acids, at least 240 amino acids, at least 250 amino acids, at least 260 amino acids, or at least 300 amino acids, or at least 350 amino acids, or up to 120 amino acids, or up to 140 amino acids, or up to 160 amino acids, or up to 180 amino acids, or up to 200 amino acids, or up to 250 amino acids, or up to 300 amino acids, or up to 350 amino acids, or up to 400 amino acids, may be truncated at the C-terminus of the Cas13 effector. Specific examples of Cas13 truncations include C-terminal Δ984-1090, C-terminal Δ1026-1090, and C-terminal Δ1053-1090, C-terminal Δ934-1090, C-terminal Δ884-1090, C-terminal Δ834-1090, C-terminal Δ784-1090, and C-terminal Δ734-1090, wherein amino acid positions correspond to amino acid positions of Prevotella sp. P5-125 Cas13b protein. The skilled person will understand that similar truncations can be designed for other Cas13b orthologs, or other Cas13 types or subtypes, such as Cas13a, Cas13c, or Cas13d. In some cases, the truncated Cas13b is encoded by nt 1-984 of Prevotella sp. P5-125 Cas13b or the corresponding nt of a Cas13b ortholog or homolog. Examples of Cas13 truncations also include C-terminal Δ795-1095, wherein amino acid positions correspond to amino acid positions of Riemerella anatipestifer Cas13b protein. Examples of Cas13 truncations further include C-terminal Δ875-1175, C-terminal Δ895-1175, C-terminal Δ915-1175, C-terminal Δ935-1175, C-terminal Δ955-1175, C-terminal Δ975-1175, C-terminal Δ995-1175, C-terminal Δ1015-1175, C-terminal Δ1035-1175, C-terminal Δ 1055-1175, C-terminal Δ1075-1175, C-terminal Δ1095-1175, C-terminal Δ1115-1175, C-terminal Δ 1135-1175, C-terminal Δ1155-1175, wherein amino acid positions correspond to amino acid positions of Porphyromonas gulae Cas13b protein.

In some embodiments, the N-terminus of the Cas13 protein may be truncated. For example, at least 20 amino acids, at least 40 amino acids, at least 50 amino acids, at least 60 amino acids, at least 80 amino acids, at least 100 amino acids, at least 120 amino acids, at least 140 amino acids, at least 150 amino acids, at least 160 amino acids, at least 180 amino acids, at least 200 amino acids, at least 220 amino acids, at least 240 amino acids, at least 250 amino acids, at least 260 amino acids, or at least 300 amino acids, or at least 350 amino acids, or up to 120 amino acids, or up to 140 amino acids, or up to 160 amino acids, or up to 180 amino acids, or up to 200 amino acids, or up to 250 amino acids, or up to 300 amino acids, or up to 350 amino acids, or up to 400 amino acids, may be truncated at the N-terminus of the Cas13 protein. Examples of Cas13 truncations include N-terminal Δ1-125, N-terminal Δ 1-88, or N-terminal Δ 1-72, wherein amino acid positions of the truncations correspond to amino acid positions of Prevotella sp. P5-125 Cas13b protein.

In some embodiments, both the N- and the C-termini of the Cas13 protein may be truncated. For example, at least 20 amino acids may be truncated at the C-terminus of the Cas13 effector, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas13 protein. For example, at least 40 amino acids may be truncated at the C-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas13 protein. For example, at least 60 amino acids may be truncated at the C-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas13 protein. For example, at least 80 amino acids may be truncated at the C-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas13 protein. For example, at least 100 amino acids may be truncated at the C-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas13 protein. For example, at least 120 amino acids may be truncated at the C-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas13 protein. For example, at least 140 amino acids may be truncated at the C-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas13 protein. For example, at least 160 amino acids may be truncated at the C-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas13 protein. For example, at least 180 amino acids may be truncated at the C-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas13 protein. For example, at least 200 amino acids may be truncated at the C-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas13 protein. For example, at least 220 amino acids may be truncated at the C-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas13 protein. For example, at least 240 amino acids may be truncated at the C-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas13 protein. For example, at least 260 amino acids may be truncated at the C-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas13 protein. For example, at least 280 amino acids may be truncated at the C-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas13 protein. For example, at least 300 amino acids may be truncated at the C-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas13 protein. For example, at least 350 amino acids may be truncated at the C-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the N-terminus of the Cas13 protein. For example, at least 20 amino acids may be truncated at the N-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas13 protein. For example, at least 40 amino acids may be truncated at the N-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas13 protein. For example, at least 60 amino acids may be truncated at the N-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas13 protein. For example, at least 80 amino acids may be truncated at the N-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas13 protein. For example, at least 100 amino acids may be truncated at the N-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas13 protein. For example, at least 120 amino acids may be truncated at the N-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas13 protein. For example, at least 140 amino acids may be truncated at the N-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas13 protein. For example, at least 160 amino acids may be truncated at the N-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas13 protein. For example, at least 180 amino acids may be truncated at the N-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas13 protein. For example, at least 200 amino acids may be truncated at the N-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas13 protein. For example, at least 220 amino acids may be truncated at the N-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas13 protein. For example, at least 240 amino acids may be truncated at the N-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas13 protein. For example, at least 260 amino acids may be truncated at the N-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas13 protein. For example, at least 280 amino acids may be truncated at the N-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas13 protein. For example, at least 300 amino acids may be truncated at the N-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas13 protein. For example, at least 350 amino acids may be truncated at the N-terminus of the Cas13 protein, and at least 20 amino acids, at least 40 amino acids, at least 60 amino acids, at least 80 amino acids, at least 100 amino acids, at least 120 amino acids, at least 140 amino acids, at least 160 amino acids, at least 180 amino acids, at least 200 amino acids, at least 220 amino acids, at least 240 amino acids, at least 260 amino acids, at least 300 amino acids, or at least 350 amino acids may be truncated at the C-terminus of the Cas13 protein.

### Split Proteins

It is noted that in this context, and more generally for the various applications as described herein, the use of a split version of the Cas protein can be envisaged. Indeed, this may not only allow increased specificity but may also be advantageous for delivery. The Cas13 is split in the sense that the two parts of the Cas13 enzyme substantially comprise a functioning Cas13. The split may be so that the catalytic domain(s) are unaffected. That Cas13 may function as a nuclease or it may be a dead-Cas13 which is essentially an RNA-binding protein with very little or no catalytic activity, due to typically mutation(s) in its catalytic domains.

Each half of the split Cas13 may be fused to a dimerization partner. By means of example, and without limitation, employing rapamycin sensitive dimerization domains, allows to generate a chemically inducible split Cas13 for temporal control of Cas13 activity. Cas13 can thus be rendered chemically inducible by being split into two fragments and that rapamycin-sensitive dimerization domains may be used for controlled reassembly of the Cas13. The two parts of the split Cas13 can be thought of as the N′ terminal part and the C′ terminal part of the split Cas13. The fusion is typically at the split point of the Cas13. In other words, the C′ terminal of the N′ terminal part of the split Cas13 is fused to one of the dimer halves, whilst the N′ terminal of the C′ terminal part is fused to the other dimer half.

The Cas13 does not have to be split in the sense that the break is newly created. The split point is typically designed in silico and cloned into the constructs. Together, the two parts of the split Cas13, the N′ terminal and C′ terminal parts, form a full Cas13, comprising preferably at least 70% or more of the wildtype amino acids (or nucleotides encoding them), preferably at least 80% or more, preferably at least 90% or more, preferably at least 95% or more, and most preferably at least 99% or more of the wildtype amino acids (or nucleotides encoding them). Some trimming may be possible, and mutants are envisaged. Non-functional domains may be removed entirely. What is important is that the two parts may be brought together and that the desired Cas13 function is restored or reconstituted. The dimer may be a homodimer or a heterodimer.

In certain embodiments, the Cas13 effector as described herein may be used for mutation-specific, or allele-specific targeting, such as. for mutation-specific, or allele-specific knockdown.

The RNA targeting effector protein can moreover be fused to another functional RNase domain, such as a non-specific RNase or Argonaute 2, which acts in synergy to increase the RNase activity or to ensure further degradation of the message.

### Functional Domains

The Cas protein or variants thereof (e.g., a catalytically inactive form) may be associated with one or more functional domains (e.g., via fusion protein or suitable linkers). In an embodiment, the Cas protein, or an ortholog or homolog thereof, may be used as a generic nucleic acid binding protein with fusion to or being operably linked to one or more functional domains. In one example, the functional domain is a deaminase. In another example, the functional domain is a transposase. In another example, the functional domain is a reverse transcriptase.

It is also envisaged that the RNA-targeting effector protein-guide RNA complex as a whole may be associated with two or more functional domains. For example, there may be two or more functional domains associated with the RNA-targeting effector protein, or there may be two or more functional domains associated with the guide RNA or crRNA (via one or more adaptor proteins), or there may be one or more functional domains associated with the RNA-targeting effector protein and one or more functional domains associated with the guide RNA or crRNA (via one or more adaptor proteins).

In some embodiments of the non-naturally occurring or engineered composition of the invention, the Cas13 effector protein is associated with one or more functional domains. The association can be by direct linkage of the effector protein to the functional domain, or by association with the crRNA. In a non-limiting example, the crRNA comprises an added or inserted sequence that can be associated with a functional domain of interest, including, for example, an aptamer or a nucleotide that binds to a nucleic acid binding adapter protein. The functional domain may be a functional heterologous domain.

In some embodiments, the invention also provides for the one or more heterologous functional domains to have one or more of the following activities: methylase activity, demethylase activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity and nucleic acid binding activity. At least one or more heterologous functional domains may be at or near the amino-terminus of the effector protein and/or wherein at least one or more heterologous functional domains is at or near the carboxy-terminus of the effector protein. The one or more heterologous functional domains may be fused to the effector protein. The one or more heterologous functional domains may be tethered to the effector protein. The one or more heterologous functional domains may be linked to the effector protein by a linker moiety.

In an embodiment, the Cas13 protein or an ortholog or homolog thereof, may be used as a generic nucleic acid binding protein with fusion to or being operably linked to a functional domain. Exemplary functional domains may include but are not limited to translational initiator, translational activator, translational repressor, nucleases, in particular ribonucleases, a spliceosome, beads, a light inducible/controllable domain or a chemically inducible/controllable domain. In certain embodiments, the one or more functional domains are controllable, e.g., inducible.

In some embodiments, one or more functional domains are associated with a Cas protein via an adaptor protein, for example as used with the modified guides of Konnerman et al. (Nature 517, 583-588, 29 Jan. 2015). In some embodiments, the one or more functional domains is attached to the adaptor protein so that upon binding of the Cas effector protein to the gRNA and target, the functional domain is in a spatial orientation allowing for the functional domain to function in its attributed function.

In some embodiments, one or more functional domains are associated with a dead gRNA (dRNA). In some embodiments, a dRNA complex with active Cas protein directs gene regulation by a functional domain at on gene locus while an gRNA directs DNA cleavage by the active Cas protein at another locus, for example as described analogously in CRISPR-Cas systems by Dahlman et al., ‘Orthogonal gene control with a catalytically active Cas9 nuclease’. In some embodiments, dRNAs are selected to maximize selectivity of regulation for a gene locus of interest compared to off-target regulation. In some embodiments, dRNAs are selected to maximize target gene regulation and minimize target cleavage

For the purposes of the following discussion, reference to a functional domain could be a functional domain associated with the Cas protein or a functional domain associated with the adaptor protein. In some embodiments, the one or more functional domains is attached to the adaptor protein so that upon binding of the Cas effector protein to the gRNA and target, the functional domain is in a spatial orientation allowing for the functional domain to function in its attributed function.

In the practice of the invention, loops of the gRNA may be extended, without colliding with the Cas protein by the insertion of distinct RNA loop(s) or distinct sequence(s) that may recruit adaptor proteins that can bind to the distinct RNA loop(s) or distinct sequence(s). The adaptor proteins may include but are not limited to orthogonal RNA-binding protein/aptamer combinations that exist within the diversity of bacteriophage coat proteins. A list of such coat proteins includes, but is not limited to: Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s and PRR1. These adaptor proteins or orthogonal RNA binding proteins can further recruit effector proteins or fusions which comprise one or more functional domains.

Examples of functional domains include deaminase domain, transposase domain, reverse transcriptase domain, integrase domain, recombinase domain, resolvase domain, invertase domain, protease domain, DNA methyltransferase domain, DNA hydroxylmethylase domain, DNA demethylase domain, histone acetylase domain, histone deacetylases domain, nuclease domain, repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase and histone tail protease. In some preferred embodiments, the functional domain is a transcriptional activation domain, such as, without limitation, VP64, p65, MyoD1, HSF1, RTA, SET7/9 or a histone acetyltransferase. In some embodiments, the functional domain is a transcription repression domain, preferably KRAB. In some embodiments, the transcription repression domain is SID, or concatemers of SID (eg SID4×). In some embodiments, the functional domain is an epigenetic modifying domain, such that an epigenetic modifying enzyme is provided. In some embodiments, the functional domain is an activation domain, which may be the P65 activation domain.

In some examples, the Cas protein is associated with a ligase or functional fragment thereof. The ligase may ligate a single-strand break (a nick) generated by the Cas protein. In certain cases, the ligase may ligate a double-strand break generated by the Cas protein. In certain examples, the Cas is associated with a reverse transcriptase or functional fragment thereof.

In some embodiments, the one or more functional domains is an NLS (Nuclear Localization Sequence) or an NES (Nuclear Export Signal). In some embodiments, the one or more functional domains is a transcriptional activation domain comprises VP64, p65, MyoD1, HSF1, RTA, SET7/9 and a histone acetyltransferase. Other references herein to activation (or activator) domains in respect of those associated with the CRISPR enzyme include any known transcriptional activation domain and specifically VP64, p65, MyoD1, HSF1, RTA, SET7/9 or a histone acetyltransferase.

In some embodiments, the one or more functional domains is a transcriptional repressor domain. In some embodiments, the transcriptional repressor domain is a KRAB domain. In some embodiments, the transcriptional repressor domain is a NuE domain, NcoR domain, SID domain or a SID4×domain.

In some embodiments, the one or more functional domains have one or more activities comprising methylase activity, demethylase activity, transcription activation activity, transcription repression activity, transcription release factor activity, histone modification activity, RNA cleavage activity, DNA cleavage activity, DNA integration activity or nucleic acid binding activity.

Histone modifying domains are also preferred in some embodiments. Exemplary histone modifying domains are discussed below. Transposase domains, HR (Homologous Recombination) machinery domains, recombinase domains, and/or integrase domains are also preferred as the present functional domains. In some embodiments, DNA integration activity includes HR machinery domains, integrase domains, recombinase domains and/or transposase domains.

In some embodiments, the DNA cleavage activity is due to a nuclease. In some embodiments, the nuclease comprises a Fok1 nuclease. See, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells.

In some embodiments, the one or more functional domains is attached to the Cas protein so that upon binding to the sgRNA and target the functional domain is in a spatial orientation allowing for the functional domain to function in its attributed function.

In particular embodiments, the Cas protein comprise one or more heterologous functional domains. As used herein, a heterologous functional domain is a polypeptide that is not derived from the same species as the Cas protein. For example, a heterologous functional domain of a Cas protein derived from species A is a polypeptide derived from a species different from species A, or an artificial polypeptide. The one or more heterologous functional domains may comprise one or more nuclear localization signal (NLS) domains. The one or more heterologous functional domains may comprise at least two or more NLSs. The one or more heterologous functional domains may comprise one or more transcriptional activation domains. A transcriptional activation domain may comprise VP64. The one or more heterologous functional domains may comprise one or more transcriptional repression domains. A transcriptional repression domain may comprise a KRAB domain or a SID domain. The one or more heterologous functional domain may comprise one or more nuclease domains. The one or more nuclease domains may comprise Fok1.

Functional domains may be used to regulate transcription, e.g., transcriptional repression. Transcriptional repression is of ten mediated by chromatin modifying enzymes such as histone methyltransferases (HMTs) and deacetylases (HDACs). Repressive histone effector domains are known and an exemplary list is provided below. In the exemplary table, preference was given to proteins and functional truncations of small size to facilitate efficient viral packaging (for instance via AAV). In general, however, the domains may include HDACs, histone methyltransferases (HMTs), and histone acetyltransferase (HAT) inhibitors, as well as HDAC and HMT recruiting proteins. The functional domain may be or include, in some embodiments, HDAC Effector Domains, HDAC Recruiter Effector Domains, Histone Methyltransferase (HMT) Effector Domains, Histone Methyltransferase (HMT) Recruiter Effector Domains, or Histone Acetyltransferase Inhibitor Effector Domains.

In some embodiments, the functional domain may be a Methyltransferase (HMT) Effector Domain. Preferred examples include NUE, vSET, EHMT2/G9A, SUV39H1, dim-5, KYP, SUVR4, SET4, SET1, SETD8, and TgSET8. NUE is exemplified in the present Examples and, although preferred, it is envisaged that others in the class will also be useful.

In some embodiments, the functional domain may be a Histone Methyltransferase (HMT) Recruiter Effector Domain. Preferred examples include Hp1a, PHF19, and NIPP1.

In some embodiments, the functional domain may be Histone Acetyltransferase Inhibitor Effector Domain. Preferred examples include SET/TAF-1β.

In some cases, the target endogenous (regulatory) control elements (such as enhancers and silencers) in addition to a promoter or promoter-proximal elements. Thus, the invention can also be used to target endogenous control elements (including enhancers and silencers) in addition to targeting of the promoter. These control elements can be located upstream and downstream of the transcriptional start site (TSS), starting from 200 bp from the TSS to 100 kb away. Targeting of known control elements can be used to activate or repress the gene of interest. In some cases, a single control element can influence the transcription of multiple target genes. Targeting of a single control element could therefore be used to control the transcription of multiple genes simultaneously.

Targeting of putative control elements on the other hand (e.g. by tiling the region of the putative control element as well as 200 bp up to 100 kB around the element) can be used as a means to verify such elements (by measuring the transcription of the gene of interest) or to detect novel control elements (e.g. by tiling 100 kb upstream and downstream of the TSS of the gene of interest). In addition, targeting of putative control elements can be useful in the context of understanding genetic causes of disease. Many mutations and common SNP variants associated with disease phenotypes are located outside coding regions. Targeting of such regions with either the activation or repression systems described herein can be followed by readout of transcription of either a) a set of putative targets (e.g. a set of genes located in closest proximity to the control element) or b) whole-transcriptome readout by e.g. RNAseq or microarray. This would allow for the identification of likely candidate genes involved in the disease phenotype. Such candidate genes could be useful as novel drug targets.

In some embodiments is for the one or more functional domains to comprise an acetyltransferase, preferably a histone acetyltransferase. These are useful in the field of epigenomics, for example in methods of interrogating the epigenome. Methods of interrogating the epigenome may include, for example, targeting epigenomic sequences. Targeting epigenomic sequences may include the guide being directed to an epigenomic target sequence. Epigenomic target sequence may include, in some embodiments, include a promoter, silencer or an enhancer sequence.

The functional domains may be acetyltransferases domains. Examples of acetyltransferases are known but may include, in some embodiments, histone acetyltransferases. In some embodiments, the histone acetyltransferase may comprise the catalytic core of the human acetyltransferase p300 (Gerbasch & Reddy, Nature Biotech 6th April 2015).

### Nuclear Localization Sequences

In some embodiments, the Cas protein is fused to one or more nuclear localization sequences (NLSs), such as about or more than about 1, 2, 3, 4, 5, 6, 7, 8, 9, 10, or more NLSs. In some embodiments, the Cas comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In a preferred embodiment of the invention, the Cas protein comprises at most 6 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Non-limiting examples of NLSs include an NLS sequence derived from: the NLS of the SV40 virus large T-antigen, having the amino acid sequence PKKKRKV (SEQ ID NO: 5205); the NLS from nucleoplasmin (e.g. the nucleoplasmin bipartite NLS with the sequence KRPAATKKAGQAKKKK (SEQ ID NO: 5206); the c-myc NLS having the amino acid sequence PAAKRVKLD (SEQ ID NO: 5207) or RQRRNELKRSP (SEQ ID NO: 5208); the hRNPA1 M9 NLS having the sequence NQSSNFGPMKGGNFGGRSSGPYGGGGQYFAKPRNQGGY (SEQ ID NO: 5209); the sequence RMRIZFKNKGKDTAELRRRRVEVSVELRKAKKDEQILKRRNV (SEQ ID NO: 5210) of the IBB domain from importin-alpha; the sequences VSRKRPRP (SEQ ID NO: 5211) and PPKKARED (SEQ ID NO: 5212) of the myoma T protein; the sequence PQPKKKPL (SEQ ID NO: 5213) of human p53; the sequence SALIKKKKKMAP (SEQ ID NO: 5214) of mouse c-abl IV; the sequences DRLRR (SEQ ID NO: 5215) and PKQKKRK (SEQ ID NO: 5216) of the influenza virus NS1; the sequence RKLKKKIKKL (SEQ ID NO: 5217) of the Hepatitis virus delta antigen; the sequence REKKKFLKRR (SEQ ID NO: 5218) of the mouse Mx1 protein; the sequence KRKGDEVDGVDEVAKKKSKK (SEQ ID NO: 5219) of the human poly(ADP-ribose) polymerase; and the sequence RKCLQAGMNLEARKTKK (SEQ ID NO: 5220) of the steroid hormone receptors (human) glucocorticoid. In general, the one or more NLSs are of sufficient strength to drive accumulation of the Cas in a detectable amount in the nucleus of a eukaryotic cell. In general, strength of nuclear localization activity may derive from the number of NLSs in the Cas, the particular NLS(s) used, or a combination of these factors. Detection of accumulation in the nucleus may be performed by any suitable technique. For example, a detectable marker may be fused to the Cas, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of CRISPR complex formation (e.g. assay for DNA cleavage or mutation at the target sequence, or assay for altered gene expression activity affected by CRISPR complex formation and/or Cas enzyme activity), as compared to a control no exposed to the Cas or complex, or exposed to a Cas lacking the one or more NLSs. In certain embodiments of the herein described Cas effector protein complexes and systems the codon optimized Cas effector proteins comprise an NLS attached to the C-terminal of the protein. In certain embodiments, other localization tags may be fused to the Cas protein, such as without limitation for localizing the Cas to particular sites in a cell, such as organelles, such as mitochondria, plastids, chloroplast, vesicles, golgi, (nuclear or cellular) membranes, ribosomes, nucleoluse, ER, cytoskeleton, vacuoles, centrosome, nucleosome, granules, centrioles, etc.

In certain embodiments of the invention, at least one nuclear localization signal (NLS) is attached to the nucleic acid sequences encoding the Cas proteins. In preferred embodiments at least one or more C-terminal or N-terminal NLSs are attached (and hence nucleic acid molecule(s) coding for the Cas protein can include coding for NLS(s) so that the expressed product has the NLS(s) attached or connected). In a preferred embodiment a C-terminal NLS is attached for optimal expression and nuclear targeting in eukaryotic cells, preferably human cells. The invention also encompasses methods for delivering multiple nucleic acid components, wherein each nucleic acid component is specific for a different target locus of interest thereby modifying multiple target loci of interest. The nucleic acid component of the complex may comprise one or more protein-binding RNA aptamers. The one or more aptamers may be capable of binding a bacteriophage coat protein.

### Linkers

In some preferred embodiments, the functional domain is linked to a dead-Cas to target and activate epigenomic sequences such as promoters or enhancers. One or more guides directed to such promoters or enhancers may also be provided to direct the binding of the CRISPR enzyme to such promoters or enhancers.

The term “associated with” is used here in relation to the association of the functional domain to the Cas effector protein or the adaptor protein. It is used in respect of how one molecule ‘associates’ with respect to another, for example between an adaptor protein and a functional domain, or between the Cas effector protein and a functional domain. In the case of such protein-protein interactions, this association may be viewed in terms of recognition in the way an antibody recognizes an epitope. Alternatively, one protein may be associated with another protein via a fusion of the two, for instance one subunit being fused to another subunit. Fusion typically occurs by addition of the amino acid sequence of one to that of the other, for instance via splicing together of the nucleotide sequences that encode each protein or subunit. Alternatively, this may essentially be viewed as binding between two molecules or direct linkage, such as a fusion protein. In any event, the fusion protein may include a linker between the two subunits of interest (i.e. between the enzyme and the functional domain or between the adaptor protein and the functional domain). Thus, in some embodiments, the Cas effector protein or adaptor protein is associated with a functional domain by binding thereto. In other embodiments, the Cas effector protein or adaptor protein is associated with a functional domain because the two are fused together, optionally via an intermediate linker.

The term “linker” as used in reference to a fusion protein refers to a molecule which joins the proteins to form a fusion protein. Generally, such molecules have no specific biological activity other than to join or to preserve some minimum distance or other spatial relationship between the proteins. However, in certain embodiments, the linker may be selected to influence some property of the linker and/or the fusion protein such as the folding, net charge, or hydrophobicity of the linker.

Suitable linkers for use in the methods of the present invention are well known to those of skill in the art and include, but are not limited to, straight or branched-chain carbon linkers, heterocyclic carbon linkers, or peptide linkers. However, as used herein the linker may also be a covalent bond (carbon-carbon bond or carbon-heteroatom bond). In particular embodiments, the linker is used to separate the Cas protein and the nucleotide deaminase by a distance sufficient to ensure that each protein retains its required functional property. Preferred peptide linker sequences adopt a flexible extended conformation and do not exhibit a propensity for developing an ordered secondary structure. In certain embodiments, the linker can be a chemical moiety which can be monomeric, dimeric, multimeric or polymeric. Preferably, the linker comprises amino acids. Typical amino acids in flexible linkers include Gly, Asn and Ser. Accordingly, in particular embodiments, the linker comprises a combination of one or more of Gly, Asn and Ser amino acids. Other near neutral amino acids, such as Thr and Ala, also may be used in the linker sequence. Exemplary linkers are disclosed in Maratea et al. (1985), Gene 40: 39-46; Murphy et al. (1986) Proc. Nat'l. Acad. Sci. USA 83: 8258-62; U.S. Pat. Nos. 4,935,233; and 4,751,180. For example, GlySer linkers GGS, GGGS (SEQ ID NO: 5221) or GSG can be used. GGS, GSG, GGGS (SEQ ID NO: 5221) or GGGGS (SEQ ID NO: 5222) linkers can be used in repeats of 3 (such as (GGS)₃ (SEQ ID NO: 5223), (GGGGS)₃ (SEQ ID NO: 5204)) or 5, 6, 7, 9 or even 12 or more, to provide suitable lengths. In some cases, the linker may be (GGGGS)₃₋₁₅, For example, in some cases, the linker may be (GGGGS)₃₋₁₁, e.g., GGGGS (SEQ ID NO: 5222), (GGGGS)₂ (SEQ ID NO: 5224, (GGGGS)₃ (SEQ ID NO: 5204), (GGGGS)₄ (SEQ ID NO: 5225), (GGGGS)₅ (SEQ ID NO: 5226), (GGGGS)₆ (SEQ ID NO: 5227), (GGGGS)₇ (SEQ ID NO: 5228), (GGGGS)₈ (SEQ ID NO: 5229), (GGGGS)₉ (SEQ ID NO: 5230), (GGGGS)₁₀ (SEQ ID NO: 5231), or (GGGGS)₁₁ (SEQ ID NO: 5232).

In particular embodiments, linkers such as (GGGGS)₃ (SEQ ID NO: 5204) are preferably used herein. (GGGGS)₆ (SEQ ID NO: 5227), (GGGGS)₉ (SEQ ID NO: 5230) or (GGGGS)₁₂ (SEQ ID NO: 5233) may preferably be used as alternatives. Other preferred alternatives are (GGGGS)₁ (SEQ ID NO: 5222), (GGGGS)₂ (SEQ ID NO:5224), (GGGGS)₄ (SEQ ID NO: 5225), (GGGGS)₅ (SEQ ID NO: 5226), (GGGGS)₇ (SEQ ID NO: 5228), (GGGGS)₈ (SEQ ID NO: 5229), (GGGGS)₁₀ (SEQ ID NO: 5231), or (GGGGS)₁₁ (SEQ ID NO: 5232). In yet a further embodiment, LEPGEKPYKCPECGKSFSQSGALTRHQRTHTR (SEQ ID NO: 5234) is used as a linker. In yet an additional embodiment, the linker is an XTEN linker. In particular embodiments, the Cas protein is linked to the deaminase protein or its catalytic domain by means of an LEPGEKPYKCPECGKSFSQSGALTRHQRTHTR (SEQ ID NO: 5234) linker. In further particular embodiments, the Cas protein is linked C-terminally to the N-terminus of a deaminase protein or its catalytic domain by means of an LEPGEKPYKCPECGKSFSQSGALTRHQRTHTR (SEQ ID NO: 5234) linker. In addition, N- and C-terminal NLSs can also function as linker (e.g., PKKKRKVEASSPKKRKVEAS (SEQ ID NO: 5235)).

Examples of linkers are shown in the Table 7 below.

TABLE 7

| | |
|---|---|
| GGS | GGTGGTAGT |
| GGSx3 (9) | GGTGGTAGTGGAGGGAGCGGCGGTTCA (SEQ ID NO: 5236) |
| GGSx7 (21) | ggtggaggaggctctggtggaggcggtagcggaggcggagggtcgGGTGGTAGTGGAGGG |
| | AGCGGCGGTTCA (SEQ ID NO: 5237) |
| XTEN | TCGGGATCTGAGACGCCTGGGACCTCGGAATCGGCTACGCCCGAA |
| | AGT (SEQ ID NO: 5238) |
| Z- | Gtggataacaaatttaacaaagaaatgtgggcggcgtgggaagaaattcgtaacctgccgaacctgaacggc |
| EGFR_Short | tggcagatgaccgcgtttattgcgagcctggtggatgatccgagccagagcgcgaacctgctggcggaagcg |
| | aaaaaactgaacgatgcgcaggcgccgaaaaccggcggtggttctggt (SEQ ID NO: 5239) |
| GSAT | Ggtggttctgccggtggctccggttctggctccagcggtggcagctctggtgcgtccggcacgggtactgcg |
| | ggtggcactggcagcggttccggtactggctctggc (SEQ ID NO: 5240) |

Linkers may be used between the guide RNAs and the functional domain (activator or repressor), or between the Cas protein and the functional domain. The linkers may be used to engineer appropriate amounts of “mechanical flexibility”.

In certain embodiments, the one or more functional domains are controllable, e.g., inducible.

### Modulation of Cas13 Proteins

The invention provides accessory proteins that modulate CRISPR protein function. In certain embodiments, the accessory protein modulates catalytic activity of a CRISPR protein. In an embodiment of the invention, an accessory protein modulates targeted, or sequence specific, nuclease activity. In an embodiment of the invention, an accessory protein modulates collateral nuclease activity. In an embodiment of the invention, an accessory protein modulates binding to a target nucleic acid.

According to the invention, the nuclease activity to be modulated can be directed against nucleic acids comprising or consisting of RNA, including without limitation mRNA, miRNA, siRNA and nucleic acids comprising cleavable RNA linkages along with nucleotide analogs. In an embodiment of the invention, the nuclease activity to be modulated can be directed against nucleic acids comprising or consisting of DNA, including without limitation nucleic acids comprising cleavable DNA linkages and nucleic acid analogs.

In an embodiment of the invention, an accessory protein enhances an activity of a CRISPR protein. In certain such embodiments, the accessory protein comprises a HEPN domain and enhances RNA cleavage. In certain embodiments, the accessory protein inhibits an activity of a CRISPR protein. In certain such embodiments, the accessory protein comprises an inactivated HEPN domain or lacks an HEPN domain altogether.

According to the invention, naturally occurring accessory proteins of Type VI CRISPR systems comprise small proteins encoded at or near a CRISPR locus that function to modify an activity of a CRISPR protein. In general, a CRISPR locus can be identified as comprising a putative CRISPR array and/or encoding a putative CRISPR effector protein. In an embodiment, an effector protein can be from 800 to 2000 amino acids, or from 900 to 1800 amino acids, or from 950 to 1300 amino acids. In an embodiment, an accessory protein can be encoded within 25 kb, or within 20 kb or within 15 kb, or within 10 kb of a putative CRISPR effector protein or array, or from 2 kb to 10 kb from a putative CRISPR effector protein or array.

In an embodiment of the invention, an accessory protein is from 50 to 300 amino acids, or from 100 to 300 amino acids or from 150 to 250 amino acids or about 200 amino acids. Non-limiting examples of accessory proteins include the csx27 and csx28 proteins identified herein.

Identification and use of a CRISPR accessory protein of the invention is independent of CRISPR effector protein classification. Accessory proteins of the invention can be found in association with or engineered to function with a variety of CRISPR effector proteins. Examples of accessory proteins identified and used herein are representative of CRISPR effector proteins generally. It is understood that CRISPR effector protein classification may involve homology, feature location (e.g., location of REC domains, NUC domains, HEPN sequences), nucleic acid target (e.g. DNA or RNA), absence or presence of tracr RNA, location of guide/spacer sequence 5′ or 3′ of a direct repeat, or other criteria. In embodiments of the invention, accessory protein identification and use transcend such classifications.

In type VI CRISPR-Cas systems that target RNA, the Cas proteins usually comprise two conserved HEPN domains which are involved in RNA cleavage. In certain embodiments, the Cas protein processes crRNA to generate mature crRNA. The guide sequence of the crRNA recognizes target RNA with a complementary sequence and the Cas protein degrades the target strand. More particularly, in certain embodiments, upon target binding, the Cas protein undergoes a structural rearrangement that brings two HEPN domains together to form an active HEPN catalytic site and the target RNA is then cleaved. The location of the catalytic site near the surface of the Cas protein allows non-specific collateral ssRNA cleavage.

In certain embodiments, accessory proteins are instrumental in increasing or reducing target and/or collateral RNA cleavage. Without being bound by theory, an accessory protein that activates CRISPR activity (e.g., a csx28 protein or ortholog or variant comprising a HEPN domain) can be envisioned as capable of interacting with a Cas protein and combining its HEPN domain with a HEPN domain of the Cas protein to form an active HEPN catalytic site, whereas an inhibitory accessory protein (e.g. csx27 with lacks an HEPN domain) can be envisioned as capable of interacting with a Cas protein and reducing or blocking a conformation of the Cas protein that would bring together two HEPN domains.

According to the invention, in certain embodiments, enhancing activity of a Type VI Cas protein or complex thereof comprises contacting the Type VI Cas protein or complex thereof with an accessory protein from the same organism that activates the Cas protein. In other embodiments, enhancing activity of a Type VI Cas protein of complex thereof comprises contacting the Type VI Cas protein or complex thereof with an activator accessory protein from a different organism within the same subclass (e.g., Type VI-b). In other embodiments, enhancing activity of a Type VI Cas protein or complex thereof comprises contacting the Type VI Cas protein or complex thereof with an accessory protein not within the subclass (e.g., a Type VI Cas protein other than Type VI-b with a Type VI-b accessory protein or vice-versa).

According to the invention, in certain embodiments, repressing activity of a Type VI Cas protein or complex thereof comprises contacting the Type VI Cas protein or complex thereof with an accessory protein from the same organism that represses the Cas protein. In other embodiments, repressing activity of a Type VI Cas protein or complex thereof comprises contacting the Type VI Cas protein or complex thereof with a repressor accessory protein from a different organism within the same subclass (e.g., Type VI-b). In other embodiments, repressing activity of a Type VI Cas protein or complex thereof comprises contacting the Type VI Cas protein or complex thereof with a repressor accessory protein not within the subclass (e.g., a Type VI Cas protein other than Type VI-b with a Type VI-b repressor accessory protein or vice-versa).

In certain embodiments where the Type VI Cas protein and the Type VI accessory protein are from the same organism, the two proteins will function together in an engineered CRISPR system. In certain embodiments, it will be desirable to alter the function of the engineered CRISPR system, for example by modifying either or both of the proteins or their expression. In embodiments where the Type VI Cas protein and the Type VI accessory protein are from different organisms which may be within the same class or different classes, the proteins may function together in an engineered CRISPR system but it will of ten be desired or necessary to modify either or both of the proteins to function together.

Accordingly, in certain embodiments of the invention either or both of a Cas protein and an accessory protein may be modified to adjust aspects of protein-protein interactions between the Cas protein and accessory protein. In certain embodiments, either or both of a Cas protein and an accessory protein may be modified to adjust aspects of protein-nucleic acid interactions. Ways to adjust protein-protein interactions and protein-nucleic acid interaction include without limitation, fitting molecular surfaces, polar interactions, hydrogen bonds, and modulating van der Waals interactions. In certain embodiments, adjusting protein-protein interactions or protein-nucleic acid binding comprises increasing or decreasing binding interactions. In certain embodiments, adjusting protein-protein interactions or protein-nucleic acid binding comprises modifications that favor or disfavor a conformation of the protein or nucleic acid.

By “fitting”, is meant determining including by automatic, or semi-automatic means, interactions between one or more atoms of a Cas13 protein (and optionally at least one atoms of a Cas13 accessory protein), or between one or more atoms of a Cas13 protein and one or more atoms of a nucleic acid, (or optionally between one or more atoms of a Cas13 accessory protein and a nucleic acid), and calculating the extent to which such interactions are stable. Interactions include attraction and repulsion, brought about by charge, steric considerations and the like.

The three-dimensional structure of Type VI CRISPR protein or complex thereof (and/or a Type VI CRISPR accessory protein or complex thereof in the context of Cas13b) provides in the context of the instant invention an additional tool for identifying additional mutations in orthologs of Cas13. The crystal structure can also be basis for the design of new and specific Cas13s (and optionally Cas13 accessory proteins). Various computer-based methods for fitting are described further. Binding interactions of Cas13s (and optionally accessory proteins), and nucleic acids can be examined through the use of computer modeling using a docking program. Docking programs are known; for example GRAM, DOCK or AUTODOCK (see Walters et al. Drug Discovery Today, vol. 3, no. 4 (1998), 160-178, and Dunbrack et al. Folding and Design 2 (1997), 27-42). This procedure can include computer fitting to ascertain how well the shape and the chemical structure of the binding partners. Computer-assisted, manual examination of the active site or binding site of a Type VI system may be performed. Programs such as GRID (P. Goodford, J. Med. Chem, 1985, 28, 849-57)-a program that determines probable interaction sites between molecules with various functional groups—may also be used to analyze the active site or binding site to predict partial structures of binding compounds. Computer programs can be employed to estimate the attraction, repulsion or steric hindrance of the two binding partners, e.g., components of a Type VI CRISPR system, or a nucleic acid molecule and a component of a Type VI CRISPR system.

Amino acid substitutions may be made on the basis of differences or similarities in amino acid properties (such as polarity, charge, solubility, hydrophobicity, hydrophilicity, and/or the amphipathic nature of the residues) and it is therefore useful to group amino acids together in functional groups. Amino acids may be grouped together based on the properties of their side chains alone. In comparing orthologs, there are likely to be residues conserved for structural or catalytic reasons. These sets may be described in the form of a Venn diagram (Livingstone C. D. and Barton G. J. (1993) “Protein sequence alignments: a strategy for the hierarchical analysis of residue conservation” Comput. Appl. Biosci. 9: 745-756) (Taylor W. R. (1986) “The classification of amino acid conservation” J. Theor. Biol. 119; 205-218). Conservative substitutions may be made, for example according to the table below which describes a generally accepted Venn diagram grouping of amino acids (see Table 8 below).

TABLE 8

| Set | | Sub-set | |
|---|---|---|---|
| Hydro- | FWYHKMILVAGC | Aromatic | FWYH |
| phobic | SEQ ID NO: 5241 | | SEQ ID NO: |
| | | | 5242 |
| | | Aliphatic | ILV |
| Polar | WYHKREDCSTNQ | Charged | HKRED |
| | SEQ ID NO: 5243 | | SEQ ID NO: |
| | | | 5244 |
| | | Positively | HKR |
| | | charged | |
| | | Negatively | ED |
| | | charged | |
| Small | VCAGSPTND | Tiny | AGS |
| | SEQ ID NO: 5245 | | |

In some embodiments, the modifications in Cas13 may comprise modification of one or more amino acid residues of the Cas13 protein (and/or may comprise modification of one or more amino acid residues of the Cas13 accessory protein). In some embodiments, the modifications in Cas13 may comprise modification of one or more amino acid residues located in a region which comprises residues which are positively charged in the unmodified Cas113 protein (and/or Cas13 accessory protein). In some embodiments, the modifications in Cas13 may comprise modification of one or more amino acid residues which are positively charged in the unmodified Cas13 protein (and/or Cas13 accessory protein). In some embodiments, the modifications in Cas13 may comprise modification of one or more amino acid residues which are not positively charged in the unmodified Cas13 protein (and/or Cas13 accessory protein). The modification may comprise modification of one or more amino acid residues which are uncharged in the unmodified Cas13 protein (and/or Cas13 accessory protein). The modification may comprise modification of one or more amino acid residues which are negatively charged in the unmodified Cas13 protein (and/or Cas13 accessory protein). The modification may comprise modification of one or more amino acid residues which are hydrophobic in the unmodified Cas13 protein (and/or Cas13 accessory protein). The modification may comprise modification of one or more amino acid residues which are polar in the unmodified Cas13 protein (and/or Cas13 accessory protein). The modification may comprise substitution of a hydrophobic amino acid or polar amino acid with a charged amino acid, which can be a negatively charged or positively charged amino acid. The modification may comprise substitution of a negatively charged amino acid with a positively charged or polar or hydrophobic amino acid. The modification may comprise substitution of a positively charged amino acid with a negatively charged or polar or hydrophobic amino acid.

Embodiments herein also include sequences (both polynucleotide or polypeptide) which may comprise homologous substitution (substitution and replacement are both used herein to mean the interchange of an existing amino acid residue or nucleotide, with an alternative residue or nucleotide) that may occur i.e., like-for-like substitution in the case of amino acids such as basic for basic, acidic for acidic, polar for polar, etc. Non-homologous substitution may also occur i.e., from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine. Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, which involves the presence of one or more amino acid residues in peptoid form, may be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D. C., Trends Biotechnol. (1995) 13(4), 132-134.

Homology modelling: Corresponding residues in other Cas13 orthologs can be identified by the methods of Zhang et al., 2012 (Nature; 490(7421): 556-60) and Chen et al., 2015 (PLoS Comput Biol; 11(5): e1004248)-a computational protein-protein interaction (PPI) method to predict interactions mediated by domain-motif interfaces. PrePPI (Predicting PPI), a structure based PPI prediction method, combines structural evidence with non-structural evidence using a Bayesian statistical framework. The method involves taking a pair query proteins and using structural alignment to identify structural representatives that correspond to either their experimentally determined structures or homology models. Structural alignment is further used to identify both close and remote structural neighbors by considering global and local geometric relationships. Whenever two neighbors of the structural representatives form a complex reported in the Protein Data Bank, this defines a template for modelling the interaction between the two query proteins. Models of a complex are created by superimposing the representative structures on their corresponding structural neighbor in the template. This approach is in Dey et al., 2013 (Prot Sci; 22: 359-66).

### Guide Sequences

The systems and compositions herein may further comprise one or more guide sequences. The guide sequences may hybridize or be capable of hybridizing with a target sequence. In embodiments of the invention the terms guide sequence and guide RNA and crRNA are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. Preferably the guide sequence is 10-30 nucleotides long, such as 30 nucleotides long. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence may be selected to target any target sequence. In some embodiments, the target sequence is a sequence within a genome of a cell. Exemplary target sequences include those that are unique in the target genome.

As used herein, the term “crRNA” or “guide RNA” or “single guide RNA” or “sgRNA” or “one or more nucleic acid components” of a Type VI CRISPR-Cas locus effector protein comprises any polynucleotide sequence having sufficient complementarity with a target nucleic acid sequence to hybridize with the target nucleic acid sequence and direct sequence-specific binding of a RNA-targeting complex to the target RNA sequence.

In some examples, the composition may comprise a Cas protein and a heterologous guide sequence, e.g., a guide sequence and the Cas protein does not exist in the same cell or the same species in nature.

In certain embodiments, the CRISPR system as provided herein can make use of a crRNA or analogous polynucleotide comprising a guide sequence, wherein the polynucleotide is an RNA, a DNA or a mixture of RNA and DNA, and/or wherein the polynucleotide comprises one or more nucleotide analogs. The sequence can comprise any structure, including but not limited to a structure of a native crRNA, such as a bulge, a hairpin or a stem loop structure. In certain embodiments, the polynucleotide comprising the guide sequence forms a duplex with a second polynucleotide sequence which can be an RNA or a DNA sequence.

In certain embodiments, guides of the invention comprise non-naturally occurring nucleic acids and/or non-naturally occurring nucleotides and/or nucleotide analogs, and/or chemically modifications. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, boranophosphate linkage, a locked nucleic acid (LNA) nucleotides comprising a methylene bridge between the 2′ and 4′ carbons of the ribose ring, or bridged nucleic acids (BNA). Other examples of modified nucleotides include 2′-O-methyl analogs, 2′-deoxy analogs, 2-thiouridine analogs, N6-methyladenosine analogs, or 2′-fluoro analogs. Further examples of modified bases include, but are not limited to, 2-aminopurine, 5-bromo-uridine, pseudouridine (ψ), N1-methylpseudouridine (me1ψ), 5-methoxyuridine(5moU), inosine, 7-methylguanosine. Examples of guide RNA chemical modifications include, without limitation, incorporation of 2′-O-methyl (M), 2′-O-methyl 3′phosphorothioate (MS), S-constrained ethyl (cEt), or 2′-O-methyl 3′thioPACE (MSP) at one or more terminal nucleotides. Such chemically modified guide RNAs can comprise increased stability and increased activity as compared to unmodified guide RNAs, though on-target vs. off-target specificity is not predictable. (See, Hendel, 2015, Nat Biotechnol. 33(9):985-9, doi: 10.1038/nbt.3290, published online 29 Jun. 2015; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Li et al., Nature Biomedical Engineering, 2017, 1, 0066 DOI:10.1038/s41551-017-0066).

In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target DNA and one or more deoxyribonucleotides and/or nucleotide analogs in a region that binds to Cas9, Cpf1, or C2cl. In an embodiment of the invention, deoxyribonucleotides and/or nucleotide analogs are incorporated in engineered guide structures, such as, without limitation, 5′ and/or 3′ end, stem-loop regions, and the seed region. In certain embodiments, the modification is not in the 5′-handle of the stem-loop regions. Chemical modification in the 5′-handle of the stem-loop region of a guide may abolish its function (see Li, et al., Nature Biomedical Engineering, 2017, 1:0066). In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl-3′-phosphorothioate (MS), S-constrained ethyl(cEt), or 2′-O-methyl-3′-thioPACE (MSP). Such modification can enhance genome editing efficiency (see Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine. In certain embodiments, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see Lee et al., eLife, 2017, 6:e25312, DOI:10.7554)

In some embodiments, the modification to the guide is a chemical modification, an insertion, a deletion or a split. In some embodiments, the chemical modification includes, but is not limited to, incorporation of 2′-O-methyl (M) analogs, 2′-deoxy analogs, 2-thiouridine analogs, N6-methyladenosine analogs, 2′-fluoro analogs, 2-aminopurine, 5-bromo-uridine, pseudouridine (ψ), N1-methylpseudouridine (me1ψ), 5-methoxyuridine(5moU), inosine, 7-methylguanosine, 2′-O-methyl-3′-phosphorothioate (MS), S-constrained ethyl(cEt), phosphorothioate (PS), or 2′-O-methyl-3′-thioPACE (MSP). In some embodiments, the guide comprises one or more of phosphorothioate modifications. In certain embodiments, at least 1, 2, 3, 4, 5, 6, 7, 8, 9, 10, 11, 12, 13, 14, 15, 16, 17, 18, 19, 20, or 25 nucleotides of the guide are chemically modified. In certain embodiments, one or more nucleotides in the seed region are chemically modified. In certain embodiments, one or more nucleotides in the 3′-terminus are chemically modified. In certain embodiments, none of the nucleotides in the 5′-handle is chemically modified. In some embodiments, the chemical modification in the seed region is a minor modification, such as incorporation of a 2′-fluoro analog. In a specific embodiment, one nucleotide of the seed region is replaced with a 2′-fluoro analog. In some embodiments, 5 or 10 nucleotides in the 3′-terminus are chemically modified. Such chemical modifications at the 3′-terminus of the Cpf1 CrRNA improve gene cutting efficiency (see Li, et al., Nature Biomedical Engineering, 2017, 1:0066). In a specific embodiment, 5 nucleotides in the 3′-terminus are replaced with 2′-fluoro analogues. In a specific embodiment, 10 nucleotides in the 3′-terminus are replaced with 2′-fluoro analogues. In a specific embodiment, 5 nucleotides in the 3′-terminus are replaced with 2′-O-methyl (M) analogs.

In some embodiments, the loop of the 5′-handle of the guide is modified. In some embodiments, the loop of the 5′-handle of the guide is modified to have a deletion, an insertion, a split, or chemical modifications. In certain embodiments, the loop comprises 3, 4, or 5 nucleotides. In certain embodiments, the loop comprises the sequence of UCUU, UUUU, UAUU, or UGUU.

In one aspect, the guide comprises portions that are chemically linked or conjugated via a non-phosphodiester bond. In one aspect, the guide comprises, in non-limiting examples, direct repeat sequence portion and a targeting sequence portion that are chemically linked or conjugated via a non-nucleotide loop. In some embodiments, the portions are joined via a non-phosphodiester covalent linker. Examples of the covalent linker include but are not limited to a chemical moiety selected from the group consisting of carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, portions of the guide are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In some embodiments, the non-targeting guide portions can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sulfonyl, ally, propargyl, diene, alkyne, and azide. Once a non-targeting portions of a guide is functionalized, a covalent chemical bond or linkage can be formed between the two oligonucleotides. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, sulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, one or more portions of a guide can be chemically synthesized. In some embodiments, the chemical synthesis uses automated, solid-phase oligonucleotide synthesis machines with 2′-acetoxyethyl orthoester (2′-ACE) (Scaringe et al., J. Am. Chem. Soc. (1998) 120: 11820-11821; Scaringe, Methods Enzymol. (2000) 317: 3-18) or 2′-thionocarbamate (2′-TC) chemistry (Dellinger et al., J. Am. Chem. Soc. (2011) 133: 11540-11546; Hendel et al., Nat. Biotechnol. (2015) 33:985-989).

In some embodiments, the guide portions can be covalently linked using various bioconjugation reactions, loops, bridges, and non-nucleotide links via modifications of sugar, internucleotide phosphodiester bonds, purine and pyrimidine residues. Sletten et al., Angew. Chem. Int. Ed. (2009) 48:6974-6998; Manoharan, M. Curr. Opin. Chem. Biol. (2004) 8: 570-9; Behlke et al., Oligonucleotides (2008) 18: 305-19; Watts, et al., Drug. Discov. Today (2008) 13: 842-55; Shukla, et al., ChemMedChem (2010) 5: 328-49.

In some embodiments, the guide portions can be covalently linked using click chemistry. In some embodiments, guide portions can be covalently linked using a triazole linker. In some embodiments, guide portions can be covalently linked using Huisgen 1,3-dipolar cycloaddition reaction involving an alkyne and azide to yield a highly stable triazole linker (He et al., ChemBioChem (2015) 17: 1809-1812; WO 2016/186745). In some embodiments, guide portions are covalently linked by ligating a 5′-hexyne portion and a 3′-azide portion. In some embodiments, either or both of the 5′-hexyne guide portion and a 3′-azide guide portion can be protected with 2′-acetoxyethyl orthoester (2′-ACE) group, which can be subsequently removed using Dharmacon protocol (Scaringe et al., J. Am. Chem. Soc. (1998) 120: 11820-11821; Scaringe, Methods Enzymol. (2000) 317: 3-18).

In some embodiments, guide portions can be covalently linked via a linker (e.g., a non-nucleotide loop) that comprises a moiety such as spacers, attachments, bioconjugates, chromophores, reporter groups, dye labeled RNAs, and non-naturally occurring nucleotide analogues. More specifically, suitable spacers for purposes of this invention include, but are not limited to, polyethers (e.g., polyethylene glycols, polyalcohols, polypropylene glycol or mixtures of ethylene and propylene glycols), polyamines group (e.g., spennine, spermidine and polymeric derivatives thereof), polyesters (e.g., poly(ethyl acrylate)), polyphosphodiesters, alkylenes, and combinations thereof. Suitable attachments include any moiety that can be added to the linker to add additional properties to the linker, such as but not limited to, fluorescent labels. Suitable bioconjugates include, but are not limited to, peptides, glycosides, lipids, cholesterol, phospholipids, diacyl glycerols and dialkyl glycerols, fatty acids, hydrocarbons, enzyme substrates, steroids, biotin, digoxigenin, carbohydrates, polysaccharides. Suitable chromophores, reporter groups, and dye-labeled RNAs include, but are not limited to, fluorescent dyes such as fluorescein and rhodamine, chemiluminescent, electrochemiluminescent, and bioluminescent marker compounds. The design of example linkers conjugating two RNA components are also described in WO 2004/015075.

The linker (e.g., a non-nucleotide loop) can be of any length. In some embodiments, the linker has a length equivalent to about 0-16 nucleotides. In some embodiments, the linker has a length equivalent to about 0-8 nucleotides. In some embodiments, the linker has a length equivalent to about 0-4 nucleotides. In some embodiments, the linker has a length equivalent to about 2 nucleotides. Example linker design is also described in WO2011/008730.

In some embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). The ability of a guide sequence (within a RNA-targeting guide RNA or crRNA) to direct sequence-specific binding of a nucleic acid-targeting complex to a target nucleic acid sequence may be assessed by any suitable assay. For example, the components of a RNA-targeting CRISPR-Cas system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible, and will occur to those skilled in the art. A guide sequence, and hence a RNA-targeting guide RNA or crRNA may be selected to target any target nucleic acid sequence. The target sequence may be DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In some embodiments, a RNA-targeting guide RNA or crRNA is selected to reduce the degree secondary structure within the RNA-targeting guide RNA or crRNA. In some embodiments, about or less than about 75%, 50%, 40%, 30%, 25%, 20%, 15%, 10%, 5%, 1%, or fewer of the nucleotides of the RNA-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P. A. Carr and G. M. Church, 2009, Nature Biotechnology 27(12): 1151-62).

In some embodiments, a nucleic acid-targeting guide is designed or selected to modulate intermolecular interactions among guide molecules, such as among stem-loop regions of different guide molecules. It will be appreciated that nucleotides within a guide that base-pair to form a stem-loop are also capable of base-pairing to form an intermolecular duplex with a second guide and that such an intermolecular duplex would not have a secondary structure compatible with CRISPR complex formation. Accordingly, is useful to select or design DR sequences in order to modulate stem-loop formation and CRISPR complex formation. In some embodiments, about or less than about 75%, 50%, 40%, 30%, 25%, 20%, 15%, 10%, 5%, 1%, or fewer of nucleic acid-targeting guides are in intermolecular duplexes. It will be appreciated that stem-loop variation will of ten be within limits imposed by DR-CRISPR effector interactions. One way to modulate stem-loop formation or change the equilibrium between stem-loop and intermolecular duplex is to vary nucleotide pairs in the stem of the stem-loop of a DR. For example, in one embodiment, a G-C pair is replaced by an A-U or U-A pair. In another embodiment, an A-U pair is substituted for a G-C or a C-G pair. In another embodiment, a naturally occurring nucleotide is replaced by a nucleotide analog. Another way to modulate stem-loop formation or change the equilibrium between stem-loop and intermolecular duplex is to modify the loop of the stem-loop of a DR. Without be bound by theory, the loop can be viewed as an intervening sequence flanked by two sequences that are complementary to each other. When that intervening sequence is not self-complementary, its effect will be to destabilize intermolecular duplex formation. The same principle applies when guides are multiplexed: while the targeting sequences may differ, it may be advantageous to modify the stem-loop region in the DRs of the different guides. Moreover, when guides are multiplexed, the relative activities of the different guides can be modulated by balancing the activity of each individual guide. In certain embodiments, the equilibrium between intermolecular stem-loops vs. intermolecular duplexes is determined. The determination may be made by physical or biochemical means and can be in the presence or absence of a CRISPR effector.

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence. In other embodiments, multiple DRs (such as dual DRs) may be present.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher. In certain embodiments, the tracrRNA may not be required. Indeed, the CRISPR-Cas effector protein from Bergeyella zoohelcum and orthologs thereof do not require a tracrRNA to ensure cleavage of an RNA target.

In further detail, the assay is as follows for a RNA target, provided that a PFS sequence is required to direct recognition. Two E. coli strains are used in this assay. One carries a plasmid that encodes the endogenous effector protein locus from the bacterial strain. The other strain carries an empty plasmid (e.g. pACYC184, control strain). All possible 7 or 8 bp PFS sequences are presented on an antibiotic resistance plasmid (pUC19 with ampicillin resistance gene). The PFS is located next to the sequence of proto-spacer 1 (the RNA target to the first spacer in the endogenous effector protein locus). Two PFS or PAM libraries were cloned. One has a 8 random bp 5′ of the proto-spacer (e.g. total of 65536 different PFS or PAM sequences=complexity). The other library has 7 random bp 3′ of the proto-spacer (e.g. total complexity is 16384 different PFSs). Both libraries were cloned to have in average 500 plasmids per possible PFS. Test strain and control strain were transformed with 5′PFS and 3′PFS library in separate transformations and transformed cells were plated separately on ampicillin plates. Recognition and subsequent cutting/interference with the plasmid renders a cell vulnerable to ampicillin and prevents growth. Approximately 12 h after transformation, all colonies formed by the test and control strains where harvested and plasmid RNA was isolated. Plasmid RNA was used as template for PCR amplification and subsequent deep sequencing. Representation of all PFSs in the untransformed libraries showed the expected representation of PFSs in transformed cells. Representation of all PFS or PAMs found in control strains showed the actual representation. Representation of all PFSs in test strain showed which PFSs are not recognized by the enzyme and comparison to the control strain allows extracting the sequence of the depleted PFS. In particular embodiments, the cleavage, such as the RNA cleavage is not PFS or PAM dependent. Indeed, for the Bergeyella zoohelcum Cas13b effector protein and its orthologs, RNA target cleavage appears to be PFS independent, and hence the Cas13 of the invention may act in a PFS or PAM independent fashion.

For minimization of toxicity and off-target effect, it will be important to control the concentration of RNA-targeting guide RNA delivered. Optimal concentrations of nucleic acid-targeting guide RNA can be determined by testing different concentrations in a cellular or non-human eukaryote animal model and using deep sequencing the analyze the extent of modification at potential off-target genomic loci. The concentration that gives the highest level of on-target modification while minimizing the level of off-target modification should be chosen for in vivo delivery. The RNA-targeting system is derived advantageously from a CRISPR-Cas system. In some embodiments, one or more elements of a RNA-targeting system is derived from a particular organism comprising an endogenous RNA-targeting system of a Cas13 proteins as herein-discussed.

### Dead Guide Sequences

In one aspect, the invention provides guide sequences which are modified in a manner which allows for formation of the CRISPR Cas complex and successful binding to the target, while at the same time, not either allowing for or not allowing for successful nuclease activity (i.e. without nuclease activity/without indel activity). For matters of explanation such modified guide sequences are referred to as “dead guides” or “dead guide sequences”. These dead guides or dead guide sequences can be thought of as catalytically inactive or conformationally inactive with regard to nuclease activity. Indeed, dead guide sequences may not sufficiently engage in productive base pairing with respect to the ability to promote catalytic activity or to distinguish on-target and off-target binding activity. Briefly, the assay involves synthesizing a CRISPR target RNA and guide RNAs comprising mismatches with the target RNA, combining these with the RNA targeting enzyme and analyzing cleavage based on gels based on the presence of bands generated by cleavage products, and quantifying cleavage based upon relative band intensities.

Hence, in a related aspect, the invention provides a non-naturally occurring or engineered composition RNA targeting CRISPR-Cas system comprising a functional RNA targeting enzyme as described herein, and guide RNA (gRNA) or crRNA wherein the gRNA or crRNA comprises a dead guide sequence whereby the gRNA is capable of hybridizing to a target sequence such that the RNA targeting CRISPR-Cas system is directed to a genomic locus of interest in a cell without detectable RNA cleavage activity of a non-mutant RNA targeting enzyme of the system. It is to be understood that any of the gRNAs or crRNAs according to the invention as described herein elsewhere may be used as dead gRNAs/crRNAs comprising a dead guide sequence.

The ability of a dead guide sequence to direct sequence-specific binding of a CRISPR complex to an RNA target sequence may be assessed by any suitable assay. For example, the components of a CRISPR-Cas system sufficient to form a CRISPR-Cas complex, including the dead guide sequence to be tested, may be provided to a host cell having the corresponding target sequence, such as by transfection with vectors encoding the components of the system, followed by an assessment of preferential cleavage within the target sequence.

As explained further herein, several structural parameters allow for a proper framework to arrive at such dead guides. Dead guide sequences can be typically shorter than respective guide sequences which result in active RNA cleavage. In particular embodiments, dead guides are 5%, 10%, 20%, 30%, 40%, 50%, shorter than respective guides directed to the same.

As explained below and known in the art, one aspect of gRNA or crRNA—RNA targeting specificity is the direct repeat sequence, which is to be appropriately linked to such guides. In particular, this implies that the direct repeat sequences are designed dependent on the origin of the RNA targeting enzyme. Structural data available for validated dead guide sequences may be used for designing CRISPR-Cas specific equivalents. Structural similarity between, e.g., the orthologous nuclease domains HEPN of two or more CRISPR-Cas effector proteins may be used to transfer design equivalent dead guides. Thus, the dead guide herein may be appropriately modified in length and sequence to reflect such CRISPR-Cas specific equivalents, allowing for formation of the CRISPR-Cas complex and successful binding to the target RNA, while at the same time, not allowing for successful nuclease activity.

Dead guides allow one to use gRNA or crRNA as a means for gene targeting, without the consequence of nuclease activity, while at the same time providing directed means for activation or repression. Guide RNA or crRNA comprising a dead guide may be modified to further include elements in a manner which allow for activation or repression of gene activity, in particular protein adaptors (e.g. aptamers) as described herein elsewhere allowing for functional placement of gene effectors (e.g. activators or repressors of gene activity). One example is the incorporation of aptamers, as explained herein and in the state of the art. By engineering the gRNA or crRNA comprising a dead guide to incorporate protein-interacting aptamers (Konermann et al., “Genome-scale transcription activation by an engineered CRISPR-Cas9 complex,” doi:10.1038/nature14136, incorporated herein by reference), one may assemble multiple distinct effector domains. Such may be modeled after natural processes.

### Prime Editing

The compositions and systems may be used for prime editing. In some embodiments, the compositions and systems may comprise a Cas protein, and RNA polymerase (e.g., RNA-dependent RNA polymerase) associated with the Cas, and a guide molecule.

In some embodiments, the Cas proteins herein may be used for prime editing. In some cases, the Cas protein may be a nickase, e.g., a RNA nickase. The Cas protein may be a dCas. In some cases, the Cas has one or more mutations. In some cases, the guide molecule may be a prime editor guide molecule.

The Cas protein may be associated with a RNA polymerase. The RNA polymerase may be fused to the C-terminus of a Cas protein. Alternatively or additionally, the RNA polymerase may be fused to the N-terminus of a Cas protein. The fusion may be via a linker and/or an adaptor protein. In some examples, the RNA polymerase may be a RNA-dependent RNA polymerase, which facilitates replication of RNA from an RNA template, e.g., the synthesis of an RNA strand complementary to a given RNA template.

The guide molecule for prime editing may be a prime editor guide molecule (also known as prime editing guide molecule) (pegRNA). In some examples, a pegRNA is a sgRNA comprising a primer binding sequence (PBS) and a template containing a desired RNA sequence (e.g., added at the 3′ end).

In some embodiments, the Cas protein herein may target DNA using a guide RNA containing a binding sequence that hybridizes to the target sequence on the DNA. The guide RNA may further comprise an editing sequence that contains new genetic information that replaces target DNA nucleotides. The small sizes of the Cas protein herein may allow easier packaging and delivery of the prime editing system, e.g., with a viral vector, e.g., AAV or lentiviral vector.

A single-strand break (a nick) may be generated on the target nucleic acid (e.g., RNA) by the Cas protein at the target site to expose a 3′-hydroxyl group, thus priming the RNA polymerase of an edit-encoding extension on the guide directly into the target site. These steps may result in a branched intermediate with two redundant single-stranded nucleic acid flaps: a 5′ flap that contains the unedited nucleic acid sequence, and a 3′ flap that contains the edited sequence copied from the guide RNA. The 5′ flaps may be removed by a structure-specific endonuclease, e.g., FEN122, which excises 5′ flaps generated during lagging-strand nucleic acid synthesis and long-patch base excision repair. The non-edited nucleic acid strand may be nicked to induce bias nucleic acid repair to preferentially replace the non-edited strand. Examples of prime editing systems and methods include those described in Anzalone AV et al., Search-and-replace genome editing without double-strand breaks or donor DNA, Nature. 2019 Oct. 21. doi: 10.1038/s41586-019-1711-4, which is incorporated by reference herein in its entirety. The reverse transcriptase in the examples may be replaced with an RNA polymerase (e.g., an RNA-dependent RNA polymerase).

The Cas protein may be used to prime-edit a single nucleotide on a target nucleic acid (e.g., RNA). Alternatively or additionally, the Cas protein may be used to prime-edit at least 2, at least 3, at least 4, at least 5, at least 6, at least 7, at least 8, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 200, at least 300, at least 400, at least 500, at least 600, at least 700, at least 800, at least 900, or at least 10000 nucleotides on a target nucleic acid.

### Diagnostic and Detection Systems

The Type VI CRISPR proteins described herein can be leveraged for CRISPR-based diagnostics (CRISPR-Dx). CRISPR-Cas can be reprogrammed with guide molecules to provide a platform for specific RNA and DNA sensing. Upon recognition of its RNA or DNA target, activated CRISPR-Cas engages in “collateral” cleavage of nearby non-targeted nucleic acids (e.g., RNA and/or ssDNA). See Abudayyeh et al. “C2c2 is a single-component programmable RNA-guided RNA-targeting CRISPR effector.” Science. Aug. 5, 2016; 353(6299); Gootenberg et al. “Nucleic acid detection with CRISPR-Cas13a/C2c2” Science. Apr. 28, 2017; 356, 438-442.

### Collateral Activity

In some embodiments, the Cas proteins possess collateral activity, that is in certain environment, an activated Cas protein remains active following binding of a target sequence and continues to non-specifically cleave non-target oligonucleotides. This guide molecule-programmed collateral cleavage activity provides an ability to use Cas13 systems to detect the presence of a specific target oligonucleotide to trigger in vivo programmed cell death or in vitro non-specific RNA degradation that can serve as a readout. (Abudayyeh et al. 2016; East-Seletsky et al, 2016).

The programmability, specificity, and collateral activity of the RNA-guided Cas13 also make it an ideal switchable nuclease for non-specific cleavage of nucleic acids. In one embodiment, a Cas13 system is engineered to provide and take advantage of collateral non-specific cleavage of nucleic acids, such as ssDNA. In another embodiment, a Cas13 system is engineered to provide and take advantage of collateral non-specific cleavage of ssDNA. Accordingly, engineered Cas13 systems may provide platforms for nucleic acid detection and transcriptome manipulation, and inducing cell death. Cas13 may be developed for use as a mammalian transcript knockdown and binding tool. Cas13 may be capable of robust collateral cleavage of RNA and ssDNA when activated by sequence-specific targeted DNA binding.

In certain embodiments, Cas13 is provided or expressed in an in vitro system or in a cell, transiently or stably, and targeted or triggered to non-specifically cleave cellular nucleic acids. In one embodiment, Cas13 is engineered to knock down ssDNA, for example viral ssDNA. In another embodiment, Cas13 is engineered to knock down RNA. The system can be devised such that the knockdown is dependent on a target DNA present in the cell or in vitro system, or triggered by the addition of a target nucleic acid to the system or cell.

In an embodiment, the Cas13 system is engineered to non-specifically cleave RNA in a subset of cells distinguishable by the presence of an aberrant DNA sequence, for instance where cleavage of the aberrant DNA might be incomplete or ineffectual. In one non-limiting example, a DNA translocation that is present in a cancer cell and drives cell transformation is targeted. Whereas a subpopulation of cells that undergoes chromosomal DNA and repair may survive, non-specific collateral ribonuclease activity advantageously leads to cell death of potential survivors.

Collateral activity was recently leveraged for a highly sensitive and specific nucleic acid detection platform termed SHERLOCK that is useful for many clinical diagnoses (Gootenberg, J. S. et al. Nucleic acid detection with CRISPR-Cas13a/C2c2. Science 356, 438-442 (2017)).

According to the invention, engineered Cas13 systems are optimized for DNA or RNA endonuclease activity and can be expressed in mammalian cells and targeted to effectively knock down reporter molecules or transcripts in cells.

The collateral effect of engineered Cas13 with isothermal amplification provides a CRISPR-based diagnostic providing rapid DNA or RNA detection with high sensitivity and single-base mismatch specificity. The Cas13-based molecular detection platform is used to detect specific strains of virus, distinguish pathogenic bacteria, genotype human DNA, and identify cell-free tumor DNA mutations. Furthermore, reaction reagents can be lyophilized for cold-chain independence and long-term storage, and readily reconstituted on paper for field applications.

The ability to rapidly detect nucleic acids with high sensitivity and single-base specificity on a portable platform may aid in disease diagnosis and monitoring, epidemiology, and general laboratory tasks. Although methods exist for detecting nucleic acids, they have trade-offs among sensitivity, specificity, simplicity, cost, and speed.

This collateral activity allows the Type VI CRISPR-Cas systems disclosed herein to detect the presence of a specific RNA or DNA in vivo by triggering programmed cell death or by nonspecific degradation of labelled RNA or ssDNA. Thus, embodiments disclosed herein include nucleic acid detection systems with high sensitivity based on nucleic acid amplification and CRISPR-Cas-mediated collateral cleavage of a labelled detection oligonucleotide, allowing for real-time detection of the target. Conservation of non-specific single stranded DNA and RNA directed proteins will inevitably lead to further and, potentially, improved CRISPR proteins that demonstrate collateral cleavage and may be used for detection and offer greater breadth for multiplexed detection of nucleic acid targets in amplified and highly sensitive, especially SHERLOCK, diagnostic systems.

In certain example embodiments, a detection system comprises a Type VI Cas protein disclosed herein and guide molecule comprising a guide sequence configured to directed binding of the CRISPR-Cas complex to a target molecule and a labeled detection molecule (“RNA-based masking construct”). Type VI and Type V Cas proteins are known to possess different cutting motif preferences. See Gootenberg et al. “Multiplexed and portable nucleic acid detection platform with Cas13b, Cas12a, and Csm6.” Science. Apr. 27, 2018, 360:439-444; International Publication WO 2019/051318. Thus, embodiments disclosed herein may further comprised multiplex embodiments comprising two or more Type VI Cas proteins with different cutting preferences, or one or more Type VI Cas proteins and one or more Type V Cas proteins. Accordingly, in such embodiments, detection molecules are configured such that each class of detection molecule is only cleaved according the cleavage preferences of one of the Type VI or Type V Cas proteins, and thus only generate a detectable signal when cleaved by the corresponding ortholog. Each ortholog is matched with a guide to a different target RNA and thus collateral activity for that ortholog is only activated when it binds its cognate target RNA and the corresponding cognate detection molecule is cleaved only when the target is bound. In this way, multiple target RNA molecules may be detected.

For ease of reference, the following section describes different RNA-based masking constructs that may be used. However, the single strand DNA equivalent for use with Type VI Cas proteins is also contemplated. In certain example embodiments, a detection construct suppresses generation of a detectable positive signal, or the RNA-based masking construct suppresses generation of a detectable positive signal by masking the detectable positive signal, or generating a detectable negative signal instead, or the RNA-based masking construct comprises a silencing RNA that suppresses generation of a gene product encoded by a reporting construct, wherein the gene product generates the detectable positive signal when expressed.

In another example embodiment, a detection construct is a ribozyme that generates a negative detectable signal, and wherein the positive detectable signal is generated when the ribozyme is deactivated. In one example embodiment, the ribozyme converts a substrate to a first color and wherein the substrate converts to a second color when the ribozyme is deactivated. In another example embodiment, the RNA-based masking agent is an aptamer that sequesters an enzyme, wherein the enzyme generates a detectable signal upon release from the aptamer by acting upon a substrate, or the aptamer sequesters a pair of agents that when released from the aptamers combine to generate a detectable signal.

In another example embodiment, the RNA-based masking construct comprises an RNA oligonucleotide to which are attached a detectable ligand oligonucleotide and a masking component. In certain example embodiments, the detectable ligand is a fluorophore and the masking component is a quencher molecule.

In another aspect, the invention provides a method for detecting target nucleic acid (e.g.,) RNAs in samples, comprising: distributing a sample or set of samples into one or more individual discrete volumes, the individual discrete volumes comprising a CRISPR system comprising an effector protein, one or more guide RNAs, an RNA-based masking construct; incubating the sample or set of samples under conditions sufficient to allow binding of the one or more guide RNAs to one or more target molecules; activating the CRISPR effector protein via binding of the one or more guide RNAs to the one or more target molecules, wherein activating the CRISPR effector protein results in modification of the RNA-based masking construct such that a detectable positive signal is produced; and detecting the detectable positive signal, wherein detection of the detectable positive signal indicates a presence of one or more target molecules in the sample.

In some embodiments, the method for detecting a target nucleic acid in a sample comprising: contacting a sample with: an engineered CRISPR-Cas protein; at least one guide polynucleotide comprising a guide sequence capable of binding to the target nucleic acid and designed to form a complex with the engineered CRISPR-Cas; and a RNA-based masking construct comprising a non-target sequence; wherein the engineered CRISPR-Cas protein exhibits collateral RNase activity and cleaves the non-target sequence of the detection construct; and detecting a signal from cleavage of the non-target sequence, thereby detecting the target nucleic acid in the sample. In some embodiments, the method further comprises contacting the sample with reagents for amplifying the target nucleic acid. In some embodiments, the reagents for amplifying comprises isothermal amplification reaction reagents. In some embodiments, the isothermal amplification reagents comprise nucleic-acid sequence-based amplification, recombinase polymerase amplification, loop-mediated isothermal amplification, strand displacement amplification, helicase-dependent amplification, or nicking enzyme amplification reagents.

In some embodiments, the target nucleic acid is DNA molecule and the method further comprises contacting the target DNA molecule with a primer comprising an RNA polymerase site and RNA polymerase.

In some embodiments, the masking construct comprises: a. a silencing RNA that suppresses generation of a gene product encoded by a reporting construct, wherein the gene product generates the detectable positive signal when expressed; b. a ribozyme that generates the negative detectable signal, and wherein the positive detectable signal is generated when the ribozyme is deactivated; or c. a ribozyme that converts a substrate to a first color and wherein the substrate converts to a second color when the ribozyme is deactivated; d. an aptamer and/or comprises a polynucleotide-tethered inhibitor; e. a polynucleotide to which a detectable ligand and a masking component are attached; f. a nanoparticle held in aggregate by bridge molecules, wherein at least a portion of the bridge molecules comprises a polynucleotide, and wherein the solution undergoes a color shift when the nanoparticle is disbursed in solution; g. a quantum dot or fluorophore linked to one or more quencher molecules by a linking molecule, wherein at least a portion of the linking molecule comprises a polynucleotide; h. a polynucleotide in complex with an intercalating agent, wherein the intercalating agent changes absorbance upon cleavage of the polynucleotide; or 1. two fluorophores tethered by a polynucleotide that undergo a shift in fluorescence when released from the polynucleotide.

In some embodiments, the aptamer a. comprises a polynucleotide-tethered inhibitor that sequesters an enzyme, wherein the enzyme generates a detectable signal upon release from the aptamer or polynucleotide-tethered inhibitor by acting upon a substrate; or b. is an inhibitory aptamer that inhibits an enzyme and prevents the enzyme from catalyzing generation of a detectable signal from a substrate or wherein the polynucleotide-tethered inhibitor inhibits an enzyme and prevents the enzyme from catalyzing generation of a detectable signal from a substrate; or c. sequesters a pair of agents that when released from the aptamers combine to generate a detectable signal.

In another aspect, the invention provides systems, compositions and methods for detecting polypeptides or polynucleotides in samples (e.g., one or more in vitro samples). Such systems or compositions may comprise a Cas protein herein; one or more detection aptamers, each designed to bind to one of the one or more target polypeptides, each detection aptamer comprising a masked promoter binding site or masked primer binding site and a trigger sequence template; and an oligonucleotide-based masking construct comprising a non-target sequence. The trigger sequence template may be used to synthesize a trigger RNA. The trigger sequence may bind to the guide molecules to activate a CRISPR system. In certain examples, the systems or compositions comprise a Cas protein herein; at least one guide polynucleotide comprising a guide sequence designed to have a degree of complementarity (e.g., at least 60%, at least 70%, at least 80%, at least 90%, at least 95%, at least 99%, or 100%) with the one or more target sequences, and designed to form a complex with the Cas protein; and an oligonucleotide-based masking construct comprising a non-target sequence, wherein the Cas protein exhibits collateral nuclease activity and cleaves the non-target sequence of the oligo-nucleotide based masking construct once activated by the one or more target sequences.

The methods may comprise: distributing a sample or set of samples into a set of individual discrete volumes, the individual discrete volumes comprising peptide detection aptamers, a CRISPR system comprising an effector protein, one or more guide RNAs, an RNA-based masking construct, wherein the peptide detection aptamers comprising a masked RNA polymerase site and configured to bind one or more target molecules; incubating the sample or set of samples under conditions sufficient to allow binding of the peptide detection aptamers to the one or more target molecules, wherein binding of the aptamer to a corresponding target molecule exposes the RNA polymerase binding site resulting in RNA synthesis of a trigger RNA; activating the CRISPR effector protein via binding of the one or more guide RNAs to the trigger RNA, wherein activating the CRISPR effector protein results in modification of the RNA-based masking construct such that a detectable positive signal is produced; and detecting the detectable positive signal, wherein detection of the detectable positive signal indicates a presence of one or more target molecules in a sample.

In certain example embodiments, the one or more guide RNAs are designed to bind to one or more target molecules that are diagnostic for a disease state. In certain other example embodiments, the disease state is an infection, an organ disease, a blood disease, an immune system disease, a cancer, a brain and nervous system disease, an endocrine disease, a pregnancy or childbirth-related disease, an inherited disease, or an environmentally-acquired disease, cancer, or a fungal infection, a bacterial infection, a parasite infection, or a viral infection.

In certain example embodiments, the RNA-based masking construct suppresses generation of a detectable positive signal, or the RNA-based masking construct suppresses generation of a detectable positive signal by masking the detectable positive signal, or generating a detectable negative signal instead, or the RNA-based masking construct comprises a silencing RNA that suppresses generation of a gene product encoded by a reporting construct, wherein the gene product generates the detectable positive signal when expressed, or the RNA-based masking construct is a ribozyme that generates the negative detectable signal, and wherein the positive detectable signal is generated when the ribozyme is inactivated. In other example embodiments, the ribozyme converts a substrate to a first state and wherein the substrate converts to a second state when the ribozyme is inactivated, or the RNA-based masking agent is an aptamer, or the aptamer sequesters an enzyme, wherein the enzyme generates a detectable signal upon release from the aptamer by acting upon a substrate, or the aptamer sequesters a pair of agents that when released from the aptamers combine to generate a detectable signal. In still further embodiments, the RNA-based masking construct comprises an RNA oligonucleotide with a detectable ligand on a first end of the RNA oligonucleotide and a masking component on a second end of the RNA oligonucleotide, or the detectable ligand is a fluorophore and the masking component is a quencher molecule.

Such systems may be further combined with amplification reagents, including isothermal amplification reagents to amplify the target DNA or RNA that when combined with the collateral effect provides assays of increased sensitivity. See Gootenberg, J. S. et al. Nucleic acid detection with CRISPR-Cas13a/C2c2. Science 356, 438-442 (2017). Isothermal amplification reagents may comprise helicase isothermal based amplification reagents (See International Application WO 2020/006036), transposase isothermal based amplification reagents (International Application WO 2020/006049) or nickase isothermal based amplification reagents (See International Publication WO 2020/006067). In an aspect, the isothermal amplification reagents may be utilized with a thermostable CRISPR-Cas protein. The combination of thermostable protein and isothermal amplification reagents may be utilized to further improve reaction times for detection and diagnostics.

Thus, the Type VI proteins, including the specific examples provided below, and CRISPR-Cas complexes disclosed herein may be further combined with a detection construct, the cleavage of which generates a detectable signal indicating detection of a target RNA by the CRISPR-Cas complex.

The ability to rapidly detect nucleic acids with high sensitivity and single-base specificity on a portable platform may aid in disease diagnosis and monitoring, epidemiology, and general laboratory tasks. Although methods exist for detecting nucleic acids, they have trade-offs among sensitivity, specificity, simplicity, cost, and speed. Further specific examples are provided below.

In another aspect, the present disclosure provides a non-naturally occurring or engineered composition comprising the Cas protein that is linked to an inactive first portion of an enzyme or reporter moiety. The enzyme or reporter moiety is reconstituted when contacted with a complementary portion of the enzyme or reporter moiety. The enzyme or reporter moiety comprises a proteolytic enzyme. In some examples, the Cas protein comprises a first Cas protein and a second Cas protein linked to the complementary portion of the enzyme or reporter moiety. Such compositions may further comprise i) a first guide capable of forming a complex with the first Cas protein and hybridizing to a first target sequence of a target nucleic acid; and ii) a second guide capable of forming a complex with the second Cas protein, and hybridizing to a second target sequence of the target nucleic acid.

### Polynucleotides and Vectors

The systems herein may comprise one or more polynucleotides. The polynucleotide(s) may comprise coding sequences of Cas protein(s), guide sequences, or any combination thereof. The present disclosure further provides vectors or vector systems comprising one or more polynucleotides herein. The vectors or vector systems include those described in the delivery sections herein.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. As used herein the term “wild type” is a term of the art understood by skilled persons and means the typical form of an organism, strain, gene or characteristic as it occurs in nature as distinguished from mutant or variant forms. A “wild type” can be a base line. As used herein the term “variant” should be taken to mean the exhibition of qualities that have a pattern that deviates from what occurs in nature. The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature. “Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y. Where reference is made to a polynucleotide sequence, then complementary or partially complementary sequences are also envisaged. These are preferably capable of hybridizing to the reference sequence under highly stringent conditions. Generally, in order to maximize the hybridization rate, relatively low-stringency hybridization conditions are selected: about 20 to 25° C. lower than the thermal melting point (Tm). The Tm is the temperature at which 50% of specific target sequence hybridizes to a perfectly complementary probe in solution at a defined ionic strength and pH. Generally, in order to require at least about 85% nucleotide complementarity of hybridized sequences, highly stringent washing conditions are selected to be about 5 to 15° C. lower than the Tm. A sequence capable of hybridizing with a given sequence is referred to as the “complement” of the given sequence.

As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this invention, it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. As used herein, “expression of a genomic locus” or “gene expression” is the process by which information from a gene is used in the synthesis of a functional gene product. The products of gene expression are of ten proteins, but in non-protein coding genes such as rRNA genes or tRNA genes, the product is functional RNA. The process of gene expression is used by all known life—eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. As used herein, the term “domain” or “protein domain” refers to a part of a protein sequence that may exist and function independently of the rest of the protein chain. As described in aspects of the invention, sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences.

In certain embodiments, the polynucleotide sequence is recombinant DNA. In further embodiments, the polynucleotide sequence further comprises additional sequences as described elsewhere herein. In certain embodiments, the nucleic acid sequence is synthesized in vitro.

Aspects of the invention relate to polynucleotide molecules that encode one or more components of the CRISPR-Cas system or Cas protein as referred to in any embodiment herein. In certain embodiments, the polynucleotide molecules may comprise further regulatory sequences. By means of guidance and not limitation, the polynucleotide sequence can be part of an expression plasmid, a minicircle, a lentiviral vector, a retroviral vector, an adenoviral or adeno-associated viral vector, a piggyback vector, or a tol2 vector. In certain embodiments, the polynucleotide sequence may be a bicistronic expression construct. In further embodiments, the isolated polynucleotide sequence may be incorporated in a cellular genome. In yet further embodiments, the isolated polynucleotide sequence may be part of a cellular genome. In further embodiments, the isolated polynucleotide sequence may be comprised in an artificial chromosome. In certain embodiments, the 5′ and/or 3′ end of the isolated polynucleotide sequence may be modified to improve the stability of the sequence of actively avoid degradation. In certain embodiments, the isolated polynucleotide sequence may be comprised in a bacteriophage. In other embodiments, the isolated polynucleotide sequence may be contained in agrobacterium species. In certain embodiments, the isolated polynucleotide sequence is lyophilized.

### Codon Optimization

Aspects of the invention relate to polynucleotide molecules that encode one or more components of one or more CRISPR-Cas systems as described in any of the embodiments herein, wherein at least one or more regions of the polynucleotide molecule may be codon optimized for expression in a eukaryotic cell. In certain embodiments, the polynucleotide molecules that encode one or more components of one or more CRISPR-Cas systems as described in any of the embodiments herein are optimized for expression in a mammalian cell or a plant cell.

An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in International Patent Publication No. WO 2014/093622 (PCT/US2013/074667) as an example of a codon optimized sequence (from knowledge in the art and this disclosure, codon optimizing coding nucleic acid molecule(s), especially as to effector protein is within the ambit of the skilled artisan). Whilst this is preferred, it will be appreciated that other examples are possible and codon optimization for a host species other than human, or for codon optimization for specific organs is known. In some embodiments, an enzyme coding sequence encoding a DNA/RNA-targeting Cas protein is codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence.

Various species exhibit particular bias for certain codons of a particular amino acid. Codon bias (differences in codon usage between organisms) of ten correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, P. A.), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid.

### BASE Editing

The present disclosure also provides for a base editing system. In general, such a system may comprise a deaminase (e.g., an adenosine deaminase or cytidine deaminase) fused with a Cas protein. The deaminase may be a full-length protein or a portion of a full-length protein that has a deaminase activity. In some examples, the Cas protein may be a mutated form of the protein of SEQ ID NOs. 1-4092, 4102-5203, and 5260-5265 or nucleic acid encoding thereof. The Cas protein may be a dead Cas protein or a Cas nickase protein. In certain examples, the system comprises a mutated form of an adenosine deaminase fused with a dead CRISPR-Cas or CRISPR-Cas nickase. The mutated form of the adenosine deaminase may have both adenosine deaminase and cytidine deaminase activities.

In one aspect, the present disclosure provides an engineered adenosine deaminase. The engineered adenosine deaminase may comprise one or more mutations herein. In some embodiments, the engineered adenosine deaminase has cytidine deaminase activity. In certain examples, the engineered adenosine deaminase has both cytidine deaminase activity and adenosine deaminase. In some cases, the modifications by base editors herein may be used for targeting post-translational signaling or catalysis.

The present disclosure also provides for base editing systems. In general, such a system may comprise a deaminase (e.g., an adenosine deaminase or cytidine deaminase) fused with a nucleic acid-guided nuclease, e.g., Cas protein. The Cas protein may be a dead Cas protein or a Cas nickase protein. In certain examples, the system comprises a mutated form of an adenosine deaminase fused with a dead CRISPR-Cas or CRISPR-Cas nickase. The mutated form of the adenosine deaminase may have both adenosine deaminase and cytidine deaminase activities.

The based editing systems may be capable of modifying a single nucleotide in a target polynucleotide. The modification may repair or correct a G→A or C→T point mutation, a T→C or A→G point mutation, or a pathogenic SNP. Accordingly, the compositions and systems may remedy a disease caused by a G→A or C→T point mutation, a T→C or A→G point mutation, or a pathogenic SNP.

In one aspect, the present disclosure provides an engineered adenosine deaminase. The engineered adenosine deaminase may comprise one or more mutations herein. In some embodiments, the engineered adenosine deaminase has cytidine deaminase activity. In certain examples, the engineered adenosine deaminase has both cytidine deaminase activity and adenosine deaminase. In some cases, the modifications by base editors herein may be used for targeting post-translational signaling or catalysis. In some embodiments, compositions herein comprise nucleotide sequence comprising encoding sequences for one or more components of a base editing system. A base-editing system may comprise a deaminase (e.g., an adenosine deaminase or cytidine deaminase) fused with a Cas protein or a variant thereof.

In another aspect, the compositions and systems have a size allowing to be packaged in a delivery particle, e.g., a virus such as AAV virus. In some examples, the present disclosure provides one or more polynucleotides encoding the Cas protein, guide sequence(s), and one or more deaminase (e.g., adenosine deaminase and its variants) in a single particle, e.g., an AAV. In a particular example, the present disclosure provides an AAV particle comprising a single vector comprising coding sequences for: (i) a small Cas13 protein (e.g., dead small Cas13b), (ii) one or more guide sequences, (iii) an adenosine deaminase.

In some cases, the adenosine deaminase is double-stranded RNA-specific adenosine deaminase (ADAR). Examples of ADARs include those described Yiannis A Savva et al., The ADAR protein family, Genome Biol. 2012; 13(12): 252, which is incorporated by reference in its entirety. In some examples, the ADAR may be hADAR1. In certain examples, the ADAR may be hADAR2. The sequence of hADAR2 may be that described under Accession No. AF525422.1.

In some cases, the deaminase may be a deaminase domain, e.g., a deaminase domain of ADAR (“ADAR-D”). In one example, the deaminase may be the deaminase domain of hADAR2 (“hADAR2-D), e.g., as described in Phelps K. J. et al., Recognition of duplex RNA by the deaminase domain of the RNA editing enzyme ADAR2. Nucleic Acids Res. 2015 January; 43(2):1123-32, which is incorporated by reference herein in its entirety. In a particular example, the hADAR2-D has a sequence comprising amino acid 299-701 of hADAR2, e.g., amino acid 299-701 of the sequence under Accession No. AF525422.1.

In certain examples, the system comprises a mutated form of an adenosine deaminase fused with a dead CRISPR-Cas or CRISPR-Cas nickase. The mutated form of the adenosine deaminase may have both adenosine deaminase and cytidine deaminase activities. In some embodiments, the adenosine deaminase may comprise one or more of the mutations: E488Q based on amino acid sequence positions of hADAR2, and mutations in a homologous ADAR protein corresponding to the above. In some embodiments, the adenosine deaminase may comprise one or more of the mutations: E488Q, V351G, based on amino acid sequence positions of hADAR2, and mutations in a homologous ADAR protein corresponding to the above. In some embodiments, the adenosine deaminase may comprise one or more of the mutations: E488Q, V351G, S486A, based on amino acid sequence positions of hADAR2, and mutations in a homologous ADAR protein corresponding to the above. In some embodiments, the adenosine deaminase may comprise one or more of the mutations: E488Q, V351G, S486A, T375S, based on amino acid sequence positions of hADAR2, and mutations in a homologous ADAR protein corresponding to the above. In some embodiments, the adenosine deaminase may comprise one or more of the mutations: E488Q, V351G, S486A, T375S, S370C, based on amino acid sequence positions of hADAR2, and mutations in a homologous ADAR protein corresponding to the above. In some embodiments, the adenosine deaminase may comprise one or more of the mutations: E488Q, V351G, S486A, T375S, S370C, P462A, based on amino acid sequence positions of hADAR2, and mutations in a homologous ADAR protein corresponding to the above. In some embodiments, the adenosine deaminase may comprise one or more of the mutations: E488Q, V351G, S486A, T375S, S370C, P462A, N597I, based on amino acid sequence positions of hADAR2, and mutations in a homologous ADAR protein corresponding to the above. In some embodiments, the adenosine deaminase may comprise one or more of the mutations: E488Q, V351G, S486A, T375S, S370C, P462A, N597I, L332I, based on amino acid sequence positions of hADAR2, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, based on amino acid sequence positions of hADAR2-D, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, based on amino acid sequence positions of hADAR2, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, based on amino acid sequence positions of hADAR2, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, based on amino acid sequence positions of hADAR2, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, based on amino acid sequence positions of hADAR2, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I based on amino acid sequence positions of hADAR2, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I, S495N based on amino acid sequence positions of hADAR2, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I, S495N, K418E based on amino acid sequence positions of hADAR2, and mutations in a homologous ADAR protein corresponding to the above. In some embodiments, the adenosine deaminase may comprise one or more of the mutations: E488Q, V351G, S486A, T375S, S370C, P462A, N597I, L332I, I398V, K350I, M383L, D619G, S582T, V440I, S495N, K418E, S661T based on amino acid sequence positions of hADAR2, and mutations in a homologous ADAR protein corresponding to the above. In some examples, provided herein includes a mutated adenosine deaminase e.g., an adenosine deaminase comprising one or more mutations of E488Q, V351G, S486A, T375S, S370C, P462A, N597I, L332I, I398V, K350I, M383L, D619G, S582T, V440I, S495N, K418E, S661T based on amino acid sequence positions of hADAR2, and mutations in a homologous ADAR protein corresponding to the above, fused with a dead CRISPR-Cas protein or CRISPR-Cas nickase. In some examples, provided herein includes a mutated adenosine deaminase e.g., an adenosine deaminase comprising E488Q, V351G, S486A, T375S, S370C, P462A, N597I, L332I, I398V, K350I, M383L, D619G, S582T, V440I, S495N, K418E, and S661T based on amino acid sequence positions of hADAR2, and mutations in a homologous ADAR protein corresponding to the above, fused with a dead CRISPR-Cas protein or a CRISPR-Cas nickase. In some examples, provided herein includes a mutated adenosine deaminase e.g., an adenosine deaminase comprising E488Q, V351G, S486A, T375S, S370C, P462A, N597I, L332I, I398V, K350I, M383L, D619G, S582T, V440I, S495N, K418E, S661T, and S375N based on amino acid sequence positions of hADAR2, and mutations in a homologous ADAR protein corresponding to the above, fused with a dead CRISPR-Cas protein or a CRISPR-Cas nickase. In some examples, provided herein includes a mutated adenosine deaminase e.g., an adenosine deaminase comprising E488Q, V351G, S486A, T375S, S370C, P462A, N597I, L332I, I398V, K350I, M383L, D619G, S582T, V440I, S495N, K418E, S661T, and S375A based on amino acid sequence positions of hADAR2, and mutations in a homologous ADAR protein corresponding to the above, fused with a dead CRISPR-Cas protein or a CRISPR-Cas nickase.

Some examples provided herein include a mutated adenosine deaminase e.g., an adenosine deaminase comprising E488Q and E620G based on amino acid sequence positions of hADAR2, and mutations in a homologous ADAR protein corresponding to the above, fused with a dead CRISPR-Cas protein or a CRISPR-Cas nickase.

Some examples provided herein include herein includes a mutated adenosine deaminase e.g., an adenosine deaminase comprising E488Q and Q696L based on amino acid sequence positions of hADAR2, and mutations in a homologous ADAR protein corresponding to the above, fused with a dead CRISPR-Cas protein or a CRISPR-Cas nickase.

Some examples provided herein include a mutated adenosine deaminase e.g., an adenosine deaminase comprising E488Q, E620G, and Q696L based on amino acid sequence positions of hADAR2, and mutations in a homologous ADAR protein corresponding to the above, fused with a dead CRISPR-Cas protein or a CRISPR-Cas nickase.

Some examples provided herein include a mutated adenosine deaminase e.g., an adenosine deaminase comprising E488Q and V505I based on amino acid sequence positions of hADAR2, and mutations in a homologous ADAR protein corresponding to the above, fused with a dead CRISPR-Cas protein or a CRISPR-Cas nickase.

In some embodiments, the adenosine deaminase may be a tRNA-specific adenosine deaminase or a variant thereof. In some embodiments, the adenosine deaminase may comprise one or more of the mutations: W23L, W23R, R26G, H36L, N37S, P48S, P48T, P48A, I49V, R51L, N72D, L84F, S97C, A106V, D108N, H123Y, G125A, A142N, S146C, D147Y, R152H, R152P, E155V, I156F, K157N, K161T, based on amino acid sequence positions of E. coli TadA, and mutations in a homologous deaminase protein corresponding to the above. In some embodiments, the adenosine deaminase may comprise one or more of the mutations: D108N based on amino acid sequence positions of E. coli TadA, and mutations in a homologous deaminase protein corresponding to the above. In some embodiments, the adenosine deaminase may comprise one or more of the mutations: A106V, D108N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, based on amino acid sequence positions of E. coli TadA, and mutations in a homologous deaminase protein corresponding to the above. In some embodiments, the adenosine deaminase may comprise one or more of the mutations: A106V, D108N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, A142N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, A142N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, A142N, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, R152P, based on amino acid sequence positions of E. coli TadA, and mutations in a homologous deaminase protein corresponding to the above. In some embodiments, the adenosine deaminase may comprise one or more of the mutations: A106V, D108N, D147Y, E155V, L84F, H123Y, I156F, H36L, R51L, S146C, K157N, P48S, W23R, P48A, R152P, A142N, based on amino acid sequence positions of E. coli TadA, and mutations in a homologous deaminase protein corresponding to the above.

In some examples, the base editing systems may comprise an intein-mediated trans-splicing system that enables in vivo delivery of a base editor, e.g., a split-intein cytidine base editors (CBE) or adenine base editor (ABE) engineered to trans-splice. Examples of the such base editing systems include those described in Colin K. W. Lim et al., Treatment of a Mouse Model of ALS by In Vivo Base Editing, Mol Ther. 2020 Jan. 14. pii: S1525-0016(20)30011-3. doi: 10.1016/j.ymthe.2020.01.005; and Jonathan M. Levy et al., Cytosine and adenine base editing of the brain, liver, retina, heart and skeletal muscle of mice via adeno-associated viruses, Nature Biomedical Engineering volume 4, pages 97-110(2020), which are incorporated by reference herein in their entireties.

In some embodiments, the base editing may introduce C-to-G edits. In some examples, the base editing system may comprise a Cas protein and a cytidine deaminase. Such system may further comprise a uracil DNA N-glycosylase. In some cases, the Cas protein is a dead Cas protein e.g., a nickase. In certain cases, the cytidine deaminase is a APOBEC1 cytidine deaminase variant, e.g., a rat APOBEC1 cytidine deaminase with R33A mutation. In certain cases, the uracil DNA N-glycosylase is derived from E coli. Such base editing system may be used to induce C-to-G modifications, e.g., in AT-rich sequence contexts in a mammalian cell (e.g., human cell).

Examples of base editing systems include those described in International Patent Publication Nos. WO 2019/071048 (e.g. paragraphs [0933]-[0938]), WO 2019/084063 (e.g., paragraphs [0173]-[0186], [0323]-[0475], [0893]-[1094]), WO 2019/126716 (e.g., paragraphs [0290]-[0425], [1077]-[1084]), WO 2019/126709 (e.g., paragraphs [0294]-[0453]), WO 2019/126762 (e.g., paragraphs [0309]-[0438]), WO 2019/126774 (e.g., paragraphs [0511]-[0670]), Cox D. B. T., et al., RNA editing with CRISPR-Cas13, Science. 2017 Nov. 24; 358(6366):1019-1027; Abudayyeh O. O., et al., A cytosine deaminase for programmable single-base RNA editing, Science 26 Jul. 2019: Vol. 365, Issue 6451, pp. 382-386; Gaudelli N. M. et al., Programmable base editing of A•T to G•C in genomic DNA without DNA cleavage, Nature volume 551, pages 464-471 (23 Nov. 2017); Komor A. C., et al., Programmable editing of a target base in genomic DNA without double-stranded DNA cleavage. Nature. 2016 May 19; 533(7603):420-4; Jordan L. Doman et al., Evaluation and minimization of Cas9-independent off-target DNA editing by cytosine base editors, Nat Biotechnol (2020). doi.org/10.1038/s41587-020-0414-6; and Richter M. F. et al., Phage-assisted evolution of an adenine base editor with improved Cas domain compatibility and activity, Nat Biotechnol (2020). doi.org/10.1038/s41587-020-0453-z, Kurt, I. C., Zhou, R., Iyer, S. et al. CRISPR C-to-G base editors for inducing targeted DNA transversions in human cells. Nat Biotechnol (2020). https://doi.org/10.1038/s41587-020-0609-x, which are incorporated by reference herein in their entireties.

### Regulation of Post-Translational Modification of Gene Products

In some cases, base editing may be used for regulating post-translational modification of a gene products. In some cases, an amino acid residue that is a post-translational modification site may be mutated by base editing to an amino residue that cannot be modified. Examples of such post-translational modifications include disulfide bond formation, glycosylation, lipidation, acetylation, phosphorylation, methylation, ubiquitination, sumoylation, or any combinations thereof.

In some embodiments, the base editors herein may regulate Stat3/IRF-5 pathway, e.g., for reduction of inflammation. For example, phosphorylation on Tyr705 of Stat3, Thr10, Ser158, Ser309, Ser317, Ser451, and/or Ser462 of IRF-5 may be involved with interleukin signaling. Base editors herein may be used to mutate one or more of these procreation sites for regulating immunity, autoimmunity, and/or inflammation.

In some embodiments, the base editors herein may regulate insulin receptor substrate (IRS) pathway. For example, phosphorylation on Ser265, Ser302, Ser325, Ser336, Ser358, Ser407, and/or Ser408 may be involved in regulating (e.g., inhibit) ISR pathway. Alternatively or additionally, Serine 307 in mouse (or Serine 312 in human) may be mutated so the phosphorylation may be regulated. For example, Serine 307 phosphorylation may lead to degradation of IRS-1 and reduce MAPK signaling. Serine 307 phosphorylation may be induced under insulin insensitivity conditions, such as insulin overstimulation and/or TNFα treatment. In some examples, S307F mutation may be generated for stabilizing the interaction between IRS-1 and other components in the pathway. Base editors herein may be used to mutate one or more of these procreation sites for regulating IRS pathway.

### Regulation of Stability of Gene Products

In some embodiments, base editing may be used for regulating the stability of gene products. For example, one or more amino acid residues that regulate protein degradation rates may be mutated by the base editors herein. In some cases, such amino acid residues may be in a degron. A degron may refer to a portion of a protein involved in regulating the degradation rate of the protein. Degrons may include short amino acid sequences, structural motifs, and exposed amino acids (e.g., lysine or arginine). Some protein may comprise multiple degrons. The degrons be ubiquitin-dependent (e.g., regulating protein degradation based on ubiquitination of the protein) or ubiquitin-independent.

In some cases, the based editing may be used to mutate one or more amino acid residues in a signal peptide for protein degradation. In some examples, the signal peptide may be a PEST sequence, which is a peptide sequence that is rich in proline (P), glutamic acid (E), serine (S), and threonine (T). For example, the stability of NANOG, which comprises a PEST sequence, may be increased, e.g., to promote embryonic stem cell pluripotency.

In some examples, the base editors may be used for mutating SMN2 (e.g., to generate S270A mutilation) to increase stability of the SMN2 protein, which is involved in spinal muscular atrophy. Other mutations in SMN2 that may be generated by based editors include those described in Cho S. et al., Genes Dev. 2010 Mar. 1; 24(5): 438-442. In certain examples, the base editors may be used for generating mutations on IκBα, as described in Fortmann K. T. et al., J Mol Biol. 2015 Aug. 28; 427(17): 2748-2756. Target sites in degrons may be identified by computational tools, e.g., the online tools provided on slim.ucd.ie/apc/index.php. Other targets include Cdc25A phosphatase.

### Examples of Genes that can be Targeted by Base Editors

In some examples, the base editors may be used for modifying PCSK9. The base editors may introduce stop codons and/or disease-associated mutations that reduce PCSK9 activity. The base editing may introduce one or more of the following mutations in PCSK9: R46L, R46A, A53V, A53A, E57K, Y142X, L253F, R237W, H391N, N425S, A443T, I474V, I474A, Q554E, Q619P, E670G, E670A, C679X, H417Q, R469W, E482G, F515L, and/or H553R.

In some examples, the base editors may be used for modifying ApoE. The base editors may target ApoE in synthetic model and/or patient-derived neurons (e.g., those derived from iPSC). The targeting may be tested by sequencing.

In some examples, the base editors may be used for modifying Stat1/3. The base editor may target Y705 and/or S727 for reducing Stat1/3 activation. The base editing may be tested by luciferase-based promoter. Targeting Stat1/3 by base editing may block monocyte to macrophage differentiation, and inflammation in response to ox-LDL stimulation of macrophages.

In some examples, the base editors may be used for modifying TFEB (transcription factor for EB). The base editor may target one or more amino acid residues that regulate translocation of the TFEB. In some cases, the base editor may target one or more amino acid residues that regulate autophagy.

In some examples, the base editors may be used for modifying ornithine carbamoyl transferase (OTC). Such modification may be used for correct ornithine carbamoyl transferase deficiency. For example, base editing may correct Leu45Pro mutation by converting nucleotide 134° C. to U.

In some examples, the base editors may be used for modifying Lipin1. The base editor may target one or more serine's that can be phosphorylated by mTOR. Base editing of Lipin1 may regulate lipid accumulation. The base editors may target Lipin1 in 3T3L1 preadipocyte model. Effects of the base editing may be tested by measuring reduction of lipid accumulation (e.g., via oil red).

A nucleotide deaminase or other RNA modification enzyme may be linked to CRISPR-Cas or a dead CRISPR-Cas via one or more amino acids. In some cases, the nucleotide deaminase may be linked to the CRISPR-Cas or a dead CRISPR-Cas via one or more amino acids 411-429, 114-124, 197-241, and 607-624. The amino acid position may correspond to a CRISPR-Cas ortholog disclosed herein. In certain examples, the nucleotide deaminase may be is linked to the dead CRISPR-Cas via one or more amino acids corresponding to amino 411-429, 114-124, 197-241, and 607-624 of Prevotella buccae CRISPR-Cas.

### Delivery

The present disclosure also provides delivery systems for introducing components of the systems and compositions herein to cells, tissues, organs, or organisms. A delivery system may comprise one or more delivery vehicles and/or cargos. Exemplary delivery systems and methods include those described in paragraphs [00117] to [00278] of Feng Zhang et al., (WO2016106236A1), and pages 1241-1251 and Table 1 of Lino C A et al., Delivering CRISPR: a review of the challenges and approaches, DRUG DELIVERY, 2018, VOL. 25, NO. 1, 1234-1257, which are incorporated by reference herein in their entireties.

In some embodiments, the delivery systems may be used to introduce the components of the systems and compositions to plant cells. For example, the components may be delivered to plant using electroporation, microinjection, aerosol beam injection of plant cell protoplasts, biolistic methods, DNA particle bombardment, and/or Agrobacterium-mediated transformation. Examples of methods and delivery systems for plants include those described in Fu et al., Transgenic Res. 2000 February; 9(1):11-9; Klein R M, et al., Biotechnology. 1992; 24:384-6; Casas A M et al., Proc Natl Acad Sci USA. 1993 Dec. 1; 90(23): 11212-11216; and U.S. Pat. No. 5,563,055, Davey M. R. et al., Plant Mol Biol. 1989 September; 13(3):273-85, which are incorporated by reference herein in their entireties.

### Cargos

The delivery systems may comprise one or more cargos. The cargos may comprise one or more components of the systems and compositions herein. A cargo may comprise one or more of the following: i) a plasmid encoding one or more Cas proteins; ii) a plasmid encoding one or more guide RNAs, iii) mRNA of one or more Cas proteins; iv) one or more guide RNAs; v) one or more Cas proteins; vi) any combination thereof. In some examples, a cargo may comprise a plasmid encoding one or more Cas protein and one or more (e.g., a plurality of) guide RNAs. In some cases, the plasmid may also encode a recombination template (e.g., for HDR). In some embodiments, a cargo may comprise mRNA encoding one or more Cas proteins and one or more guide RNAs.

In some examples, a cargo may comprise one or more Cas proteins and one or more guide RNAs, e.g., in the form of ribonucleoprotein complexes (RNP). The ribonucleoprotein complexes may be delivered by methods and systems herein. In some cases, the ribonucleoprotein may be delivered by way of a polypeptide-based shuttle agent. In one example, the ribonucleoprotein may be delivered using synthetic peptides comprising an endosome leakage domain (ELD) operably linked to a cell penetrating domain (CPD), to a histidine-rich domain and a CP. D., e.g., as describe in WO2016161516. RNP may also be used for delivering the compositions and systems to plant cells, e.g., as described in Wu J. W., et al., Nat Biotechnol. 2015 November; 33(11):1162-4.

### Physical Delivery

In some embodiments, the cargos may be introduced to cells by physical delivery methods. Examples of physical methods include microinjection, electroporation, and hydrodynamic delivery. Both nucleic acid and proteins may be delivered using such methods. For example, Cas protein may be prepared in vitro, isolated, (refolded, purified if needed), and introduced to cells.

### Microinjection

Microinjection of the cargo directly to cells can achieve high efficiency, e.g., above 90% or about 100%. In some embodiments, microinjection may be performed using a microscope and a needle (e.g., with 0.5-5.0 m in diameter) to pierce a cell membrane and deliver the cargo directly to a target site within the cell. Microinjection may be used for in vitro and ex vivo delivery.

Plasmids comprising coding sequences for Cas proteins and/or guide RNAs, mRNAs, and/or guide RNAs, may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery sgRNA directly to the nucleus and Cas-encoding mRNA to the cytoplasm, e.g., facilitating translation and shuttling of Cas to the nucleus.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up- or down-regulate a specific gene within the genome of a cell, e.g., using CRISPRa and CRISPRi.

### Electroporation

In some embodiments, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S. R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses, non-viral vehicles, and other delivery reagents described herein.

The delivery vehicles in accordance with the present invention may have a greatest dimension (e.g. diameter) of less than 100 microns (μm). In some embodiments, the delivery vehicles have a greatest dimension of less than 10 μm. In some embodiments, the delivery vehicles may have a greatest dimension of less than 2000 nanometers (nm). In some embodiments, the delivery vehicles may have a greatest dimension of less than 1000 nanometers (nm). In some embodiments, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension ranging between 25 nm and 200 nm.

In some embodiments, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles). Nanoparticles may also be used to deliver the compositions and systems to plant cells, e.g., as described in International Patent Publication No. WO 2008042156, US Publication Application No. US 20130185823, and International Patent Publication No WO 2015/089419.

### Vectors

The systems, compositions, and/or delivery systems may comprise one or more vectors. The present disclosure also includes vector systems. A vector system may comprise one or more vectors. In some embodiments, a vector refers to a nucleic acid molecule capable of transporting another nucleic acid to which it has been linked. Vectors include nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques. Certain vectors may be capable of autonomous replication in a host cell into which they are introduced (e.g., bacterial vectors having a bacterial origin of replication and episomal mammalian vectors). Some vectors (e.g., non-episomal mammalian vectors) are integrated into the genome of a host cell upon introduction into the host cell, and thereby are replicated along with the host genome. In certain examples, vectors may be expression vectors, e.g., capable of directing the expression of genes to which they are operatively-linked. In some cases, the expression vectors may be for expression in eukaryotic cells. Common expression vectors of utility in recombinant DNA techniques are of ten in the form of plasmids.

Examples of vectors include pGEX, pMAL, pRIT5, E. coli expression vectors (e.g., pTrc, pET 11d, yeast expression vectors (e.g., pYepSec1, pMFa, pJRY88, pYES2, and picZ, Baculovirus vectors (e.g., for expression in insect cells such as SF9 cells) (e.g., pAc series and the pVL series), mammalian expression vectors (e.g., pCDM8 and pMT2PC.

A vector may comprise i) Cas encoding sequence(s), and/or ii) a single, or at least 2, at least 3, at least 4, at least 5, at least 6, at least 7, at least 8, at least 9, at least 10, at least 12, at least 14, at least 16, at least 32, at least 48, at least 50 guide RNA(s) encoding sequences. In a single vector there can be a promoter for each RNA coding sequence. Alternatively or additionally, in a single vector, there may be a promoter controlling (e.g., driving transcription and/or expression) multiple RNA encoding sequences.

Furthermore, that compositions or systems may be delivered via a vector, e.g., a separate vector or the same vector that is encoding the CRISPR complex. When provided by a separate vector, the CRISPR RNA that targets Cas expression can be administered sequentially or simultaneously. When administered sequentially, the CRISPR RNA that targets Cas expression is to be delivered after the CRISPR RNA that is intended for e.g. gene editing or gene engineering. This period may be a period of minutes (e.g. 5 minutes, 10 minutes, 20 minutes, 30 minutes, 45 minutes, 60 minutes). This period may be a period of hours (e.g. 2 hours, 4 hours, 6 hours, 8 hours, 12 hours, 24 hours). This period may be a period of days (e.g. 2 days, 3 days, 4 days, 7 days). This period may be a period of weeks (e.g. 2 weeks, 3 weeks, 4 weeks). This period may be a period of months (e.g. 2 months, 4 months, 8 months, 12 months). This period may be a period of years (2 years, 3 years, 4 years). In this fashion, the Cas enzyme associates with a first gRNA capable of hybridizing to a first target, such as a genomic locus or loci of interest and undertakes the function(s) desired of the CRISPR-Cas system (e.g., gene engineering); and subsequently the Cas enzyme may then associate with the second gRNA capable of hybridizing to the sequence comprising at least part of the Cas or CRISPR cassette. Where the guide RNA targets the sequences encoding expression of the Cas protein, the enzyme becomes impeded and the system becomes self-inactivating. In the same manner, CRISPR RNA that targets Cas expression applied via, for example liposome, lipofection, particles, microvesicles as explained herein, may be administered sequentially or simultaneously. Similarly, self-inactivation may be used for inactivation of one or more guide RNA used to target one or more targets.

### Regulatory Elements

A vector may comprise one or more regulatory elements. The regulatory element(s) may be operably linked to coding sequences of Cas proteins, accessary proteins, guide RNAs (e.g., a single guide RNA, crRNA, and/or tracrRNA), or combination thereof. The term “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). In certain examples, a vector may comprise: a first regulatory element operably linked to a nucleotide sequence encoding a Cas protein, and a second regulatory element operably linked to a nucleotide sequence encoding a guide RNA.

Examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific.

Examples of promoters include one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1α promoter.

### Viral Vectors

The cargos may be delivered by viruses. In some embodiments, viral vectors are used. A viral vector may comprise virally-derived DNA or RNA sequences for packaging into a virus (e.g., retroviruses, replication defective retroviruses, adenoviruses, replication defective adenoviruses, and adeno-associated viruses). Viral vectors also include polynucleotides carried by a virus for transfection into a host cell. Viruses and viral vectors may be used for in vitro, ex vivo, and/or in vivo deliveries.

### Adeno Associated Virus (AAV)

The systems and compositions herein may be delivered by adeno associated virus (AAV). AAV vectors may be used for such delivery. AAV, of the Dependovirus genus and Parvoviridae family, is a single stranded DNA virus. In some embodiments, AAV may provide a persistent source of the provided DNA, as AAV delivered genomic material can exist indefinitely in cells, e.g., either as exogenous DNA or, with some modification, be directly integrated into the host DNA. In some embodiments, AAV do not cause or relate with any diseases in humans. The virus itself is able to efficiently infect cells while provoking little to no innate or adaptive immune response or associated toxicity.

Examples of AAV that can be used herein include AAV-1, AAV-2, AAV-3, AAV-4, AAV-5, AAV-6, AAV-8, and AAV-9. The type of AAV may be selected with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. AAV-2-based vectors were originally proposed for CFTR delivery to CF airways, other serotypes such as AAV-1, AAV-5, AAV-6, and AAV-9 exhibit improved gene transfer efficiency in a variety of models of the lung epithelium. Examples of cell types targeted by AAV are described in Grimm, D. et al, J. Virol. 82: 5887-5911 (2008)), and shown as follows:

TABLE 9

| | AAV- | AAV- | AAV- | AAV- | AAV- | AAV- | AAV- | AAV- |
|---|---|---|---|---|---|---|---|---|
| Cell Line | 1 | 2 | 3 | 4 | 5 | 6 | 8 | 9 |
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HIT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

CRISPR-Cas AAV particles may be created in HEK 293 T cells. Once particles with specific tropism have been created, they are used to infect the target cell line much in the same way that native viral particles do. This may allow for persistent presence of CRISPR-Cas components in the infected cell type, and what makes this version of delivery particularly suited to cases where long-term expression is desirable. Examples of doses and formulations for AAV that can be used include those describe in U.S. Pat. Nos. 8,454,972 and 8,404,658.

Various strategies may be used for delivery the systems and compositions herein with AAVs. In some examples, coding sequences of Cas and gRNA may be packaged directly onto one DNA plasmid vector and delivered via one AAV particle. In some examples, AAVs may be used to deliver gRNAs into cells that have been previously engineered to express Cas. In some examples, coding sequences of Cas and gRNA may be made into two separate AAV particles, which are used for co-transfection of target cells. In some examples, markers, tags, and other sequences may be packaged in the same AAV particles as coding sequences of Cas and/or gRNAs.

### Lentiviruses

The systems and compositions herein may be delivered by lentiviruses. Lentiviral vectors may be used for such delivery. Lentiviruses are complex retroviruses that have the ability to infect and express their genes in both mitotic and post-mitotic cells.

Examples of lentiviruses include human immunodeficiency virus (HIV), which may use its envelope glycoproteins of other viruses to target a broad range of cell types; minimal non-primate lentiviral vectors based on the equine infectious anemia virus (EIAV), which may be used for ocular therapies. In certain embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) may be used/and or adapted to the nucleic acid-targeting system herein.

Lentiviruses may be pseudo-typed with other viral proteins, such as the G protein of vesicular stomatitis virus. In doing so, the cellular tropism of the lentiviruses can be altered to be as broad or narrow as desired. In some cases, to improve safety, second- and third-generation lentiviral systems may split essential genes across three plasmids, which may reduce the likelihood of accidental reconstitution of viable viral particles within cells.

In some examples, leveraging the integration ability, lentiviruses may be used to create libraries of cells comprising various genetic modifications, e.g., for screening and/or studying genes and signaling pathways.

### Adenoviruses

The systems and compositions herein may be delivered by adenoviruses. Adenoviral vectors may be used for such delivery. Adenoviruses include nonenveloped viruses with an icosahedral nucleocapsid containing a double stranded DNA genome. Adenoviruses may infect dividing and non-dividing cells. In some embodiments, adenoviruses do not integrate into the genome of host cells, which may be used for limiting off-target effects of CRISPR-Cas systems in gene editing applications.

### Viral Vehicles for Delivery to Plants

The systems and compositions may be delivered to plant cells using viral vehicles. In particular embodiments, the compositions and systems may be introduced in the plant cells using a plant viral vector (e.g., as described in Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

### Non-Viral Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, gold nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes.

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of Cas and/or gRNA) and/or RNA molecules (e.g., mRNA of Cas, gRNAs). In certain cases, LNPs may be use for delivering RNP complexes of Cas/gRNA.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200, December 2011).

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMA)

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

### Lipoplexes/Polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2 (e.g., forming DNA/Ca²⁺ microcomplexes), polyethenimine (PEI) (e.g., branched PEI), and poly(L-lysine) (PLL).

### Cell Penetrating Peptides

In some embodiments, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas protein directly, which is then complexed with the gRNA and delivered to cells. In some examples, separate delivery of CPP-Cas and CPP-gRNA to multiple cells may be performed. CPP may also be used to delivery RNPs.

CPPs may be used to deliver the compositions and systems to plants. In some examples, CPPs may be used to deliver the components to plant protoplasts, which are then regenerated to plant cells and further to plants.

### DNA Nanoclews

In some embodiments, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. DNA nanoclew may have a palindromic sequence to be partially complementary to the gRNA within the Cas:gRNA ribonucleoprotein complex. A DNA nanoclew may be coated, e.g., coated with PEI to induce endosomal escape.

### Gold Nanoparticles

In some embodiments, the delivery vehicles comprise gold nanoparticles (also referred to AuNPs or colloidal gold). Gold nanoparticles may form complex with cargos, e.g., Cas:gRNA RNP. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901.

iTOP

In some embodiments, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D. S., Pagliero R. J., Pras A, et al. (2015). Cell 161:674-690.

### Polymer-Based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions herein include those described in Bawage S. S. et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data., doi:10.13140/RG.2.2.23912.16642.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K. W., et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P. N., et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; E1-Andaloussi S, et al., Nat Protoc. 2012 December; 7(12):2112-26; Uno Y, et al., Hum Gene Ther. 2011 June; 22(6):711-9; Zou W, et al., Hum Gene Ther. 2011 April; 22(4):465-75.

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

### Optimized Functional RNA Targeting Systems

In an aspect the invention thus provides a system for specific delivery of functional components to the RNA environment. This can be ensured using the CRISPR systems comprising the RNA targeting effector proteins of the present invention which allow specific targeting of different components to RNA. More particularly such components include activators or repressors, such as activators or repressors of RNA translation, degradation, etc. CRISPR-Cas13 knockdown allows for temporary reduction of gene expression through the use of artificial transcription factors, e.g., via mutating residues in cleavage domain(s) of the Cas13 protein results in the generation of a catalytically inactive Cas13 protein. A catalytically inactive Cas13 complexes with a guide RNA or crRNA and localizes to the RNA sequence specified by that guide RNA's or crRNA's targeting domain, however, it does not cleave the target. Fusion of the inactive Cas13 protein to an effector domain, e.g., a transcription repression domain, enables recruitment of the effector to any site specified by the guide RNA.

According to one aspect the invention provides non-naturally occurring or engineered composition comprising a guide RNA or crRNA comprising a guide sequence capable of hybridizing to a target sequence of interest in a cell, wherein the guide RNA or crRNA is modified by the insertion of one or more distinct RNA sequence(s) that bind an adaptor protein. In particular embodiments, the RNA sequences may bind to two or more adaptor proteins (e.g. aptamers), and wherein each adaptor protein is associated with one or more functional domains. When there is more than one functional domain, the functional domains can be same or different, e.g., two of the same or two different activators or repressors. In an aspect the invention provides a herein-discussed composition, wherein the one or more functional domains are attached to the RNA targeting enzyme so that upon binding to the target RNA the functional domain is in a spatial orientation allowing for the functional domain to function in its attributed function; In an aspect the invention provides a herein-discussed composition, wherein the composition comprises a CRISPR-Cas13 complex having at least three functional domains, at least one of which is associated with the RNA targeting enzyme and at least two of which are associated with the gRNA or crRNA.

### Genetically Modified Cells and Organisms

The present disclosure further provides cells comprising one or more components of the systems herein, e.g., the Cas protein and/or guide molecule(s). Also provided include cells modified by the systems and methods herein, and cell cultures, tissues, organs, organism comprising such cells or progeny thereof. The invention in some embodiments comprehends a method of modifying an cell or organism. The cell may be a prokaryotic cell or a eukaryotic cell. The cell may be a mammalian cell. The mammalian cell many be a non-human primate, bovine, porcine, rodent or mouse cell. The cell may be a non-mammalian eukaryotic cell such as poultry, fish or shrimp. The cell may be a therapeutic T cell or antibody-producing B-cell. The cell may also be a plant cell. The plant cell may be of a crop plant such as cassava, corn, sorghum, wheat, or rice. The plant cell may also be of an algae, tree or vegetable. The modification introduced to the cell by the present invention may be such that the cell and progeny of the cell are altered for improved production of biologic products such as an antibody, starch, alcohol or other desired cellular output. The modification introduced to the cell by the present invention may be such that the cell and progeny of the cell include an alteration that changes the biologic product produced.

In some embodiments, one or more polynucleotide molecules, vectors, or vector systems driving expression of one or more elements of a nucleic acid-targeting system or delivery systems comprising one or more elements of the nucleic acid-targeting system are introduced into a host cell such that expression of the elements of the nucleic acid-targeting system direct formation of a nucleic acid-targeting complex at one or more target sites. In certain embodiments of the invention the host cell may be a eukaryotic cell, a prokaryotic cell, or a plant cell.

In particular embodiments, the host cell is a cell of a cell line. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

Further intended are isolated human cells or tissues, plants or non-human animals comprising one or more of the polynucleotide molecules, vectors, vector systems, or cells described in any of the embodiments herein. In an aspect, host cells and cell lines modified by or comprising the compositions, systems or modified enzymes of present invention are provided, including (isolated) stem cells, and progeny thereof.

In certain embodiments, the plants or non-human animals comprise at least one of the CRISPR system components, polynucleotide molecules, vectors, vector systems, or cells described in any of the embodiments herein at least one tissue type of the plant or non-human animal. In certain embodiments, non-human animals comprise at least one of the CRISPR system components, polynucleotide molecules, vectors, vector systems, or cells described in any of the embodiments herein in at least one tissue type. In certain embodiments, the presence of the CRISPR system components is transient, in that they are degraded over time. In certain embodiments, expression of the CRISPR-Cas systems or Cas proteins described in any of the embodiments comprised in polynucleotide molecules, vectors, vector systems, or cells is limited to certain tissue types or regions in the plant or non-human animal. In certain embodiments, the expression of the CRISPR-Cas systems or Cas proteins described in any of the embodiments comprised in polynucleotide molecules, vectors, vector systems, or cells is dependent of a physiological cue. In certain embodiments, expression of the CRISPR-Cas systems or Cas proteins described in any of the embodiments comprised in polynucleotide molecules, vectors, vector systems, or cells may be triggered by an exogenous molecule. In certain embodiments, expression of the CRISPR-Cas systems or Cas proteins described in any of the embodiments comprised in polynucleotide molecules, vectors, vector systems, or cells is dependent on the expression of a non-cas molecule in the plant or non-human animal.

### Methods of Use in General

In another aspect, the present disclosure discloses methods of using the compositions and systems herein. In general, the methods include modifying a target nucleic acid by introducing in a cell or organism that comprises the target nucleic acid the engineered Cas protein, polynucleotide(s) encoding engineered Cas protein, the CRISPR-Cas system, or the vector or vector system comprising the polynucleotide(s), such that the engineered CRISPR-Cas protein modifies the target nucleic acid in the cell or organism. The engineered CRISPR-Cas protein or system may be introduced via delivery by liposomes, nanoparticles, exosomes, microvesicles, nucleic acid nanoassemblies, a gene gun, an implantable device, or the vector system herein. The cell or organisms may be a eukaryotic cell or organism. The cell or organisms is an animal cell or organism. The cell or organisms is a plant cell or organism. Examples of nucleic acid nanoassemblies include DNA origami and RNA origami, e.g., those described in U.S. Pat. No. 8,554,489, US20160103951, WO2017189914, and WO2017189870, which are incorporated by reference in their entireties. A gene gun may include a biolistic particle delivery system, which is a device for delivering exogenous DNA (transgenes) to cells. The payload may be an elemental particle of a heavy metal coated with DNA (typically plasmid DNA). An example of delivery components in CRISPR-Cas systems is described in Svitashev et al., Nat Commun. 2016; 7: 13274.

In some embodiments, the target nucleic acid comprises a genomic locus, and the engineered CRISPR-Cas protein modifies gene product encoded at the genomic locus or expression of the gene product. The target nucleic acid is DNA or RNA and wherein one or more nucleotides in the target nucleic acid may be base edited. The target nucleic acid may be DNA or RNA and wherein the target nucleic acid is cleaved. The engineered CRISPR-Cas protein may further cleave non-target nucleic acid.

### Non-Homologous End-Joining

In certain embodiments, nuclease-induced non-homologous end-joining (NHEJ) can be used to target gene-specific knockouts. Nuclease-induced NHEJ can also be used to remove (e.g., delete) sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. Two-thirds of these mutations typically alter the reading frame and, therefore, produce a non-functional protein. Additionally, mutations that maintain the reading frame, but which insert or delete a significant amount of sequence, can destroy functionality of the protein. This is locus dependent as mutations in critical functional domains are likely less tolerable than mutations in non-critical regions of the protein. The indel mutations generated by NHEJ are unpredictable in nature; however, at a given break site certain indel sequences are favored and are over represented in the population, likely due to small regions of microhomology. The lengths of deletions can vary widely; most commonly in the 1-50 bp range, but they can easily be greater than 50 bp, e.g., they can easily reach greater than about 100-200 bp. Insertions tend to be shorter and of ten include short duplications of the sequence immediately surrounding the break site. However, it is possible to obtain large insertions, and in these cases, the inserted sequence has of ten been traced to other regions of the genome or to plasmid DNA present in the cells.

Because NHEJ is a mutagenic process, it may also be used to delete small sequence motifs as long as the generation of a specific final sequence is not required. If a double-strand break is targeted near to a short target sequence, the deletion mutations caused by the NHEJ repair of ten span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences; however, the error-prone nature of NHEJ may still produce indel mutations at the site of repair.

Both double strand cleaving Cas proteins, or an ortholog or homolog thereof, and single strand, or nickase, Cas proteins, or an ortholog or homolog thereof, molecules can be used in the methods and compositions described herein to generate NHEJ-mediated indels. NHEJ-mediated indels targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp).

In an embodiment, in which a guide RNA and Cas protein, or an ortholog or homolog thereof, generate a double strand break for the purpose of inducing NHEJ-mediated indels, a guide RNA may be configured to position one double-strand break in close proximity to a nucleotide of the target position. In an embodiment, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position).

In an embodiment, in which two guide RNAs complexing with Cas proteins, or an ortholog or homolog thereof, preferably Cas nickases induce two single strand breaks for the purpose of inducing NHEJ-mediated indels, two guide RNAs may be configured to position two single-strand breaks to provide for NHEJ repair a nucleotide of the target position.

In some examples, the systems herein may introduce one or more indels via NHEJ pathway and insert sequence from a combination template via HDR.

### Diagnostic Uses

In some embodiments, the methods may further comprise visualizing activity and, optionally, using a detectable label. The method may also comprise detecting binding of one or more components of the CRISPR-Cas system to the target nucleic acid.

In another aspect the methods of use include detecting a target nucleic acid in a sample. In some embodiments, the methods include contacting a sample with: an engineered CRISPR-Cas protein herein; at least one guide polynucleotide comprising a guide sequence capable of binding to the target nucleic acid and designed to form a complex with the engineered CRISPR-Cas; and a RNA-based masking construct comprising a non-target sequence; wherein the engineered CRISPR-Cas protein exhibits collateral RNase activity and cleaves the non-target sequence of the detection construct; and detecting a signal from cleavage of the non-target sequence, thereby detecting the target nucleic acid in the sample. The methods may further comprise contacting the sample with reagents for amplifying the target nucleic acid. The reagents for amplifying may comprise isothermal amplification reaction reagents. The isothermal amplification reagents may comprise nucleic-acid sequence-based amplification, recombinase polymerase amplification, loop-mediated isothermal amplification, strand displacement amplification, helicase-dependent amplification, or nicking enzyme amplification reagents. The target nucleic acid is DNA molecule and the method may further comprise contacting the target DNA molecule with a primer comprising an RNA polymerase site and RNA polymerase.

Detection can comprise two or more detection systems utilizing RNA targeting Cas effector proteins; DNA targeting Cas effector proteins, or a combination thereof. The RNA-targeting effector proteins may be a Cas13 protein, such as Cas13a, Cas13b, or Cas13c. The DNA-targeting effector protein may be a Type VI protein, e.g. Cas12 protein such as Cpf1 and C2c1. Multiplexing systems can be designed such that different Cas proteins with different sequence specificities or other motif cutting preferences can be used. See International Publication WO 2019/126577. Multiplex approaches and selection of Cas effector proteins can be as described in International Publication WO 2019/126577 at [0415]—[0416] and Examples 1-10, incorporated herein by reference.

The masking construct: suppresses generation of a detectable positive signal until the masking construct cleaved or deactivated, or masks a detectable positive signal or generates a detectable negative signal until the masking construct cleaved or deactivated. The masking construct may comprise: a silencing RNA that suppresses generation of a gene product encoded by a reporting construct, wherein the gene product generates the detectable positive signal when expressed; a ribozyme that generates the negative detectable signal, and wherein the positive detectable signal is generated when the ribozyme is deactivated; or a ribozyme that converts a substrate to a first color and wherein the substrate converts to a second color when the ribozyme is deactivated; an aptamer and/or comprises a polynucleotide-tethered inhibitor; a polynucleotide to which a detectable ligand and a masking component are attached; a nanoparticle held in aggregate by bridge molecules, wherein at least a portion of the bridge molecules comprises a polynucleotide, and wherein the solution undergoes a color shift when the nanoparticle is disbursed in solution; a quantum dot or fluorophore linked to one or more quencher molecules by a linking molecule, wherein at least a portion of the linking molecule comprises a polynucleotide; a polynucleotide in complex with an intercalating agent, wherein the intercalating agent changes absorbance upon cleavage of the polynucleotide; or two fluorophores tethered by a polynucleotide that undergo a shift in fluorescence when released from the polynucleotide.

The aptamer may comprise a polynucleotide-tethered inhibitor that sequesters an enzyme, wherein the enzyme generates a detectable signal upon release from the aptamer or polynucleotide-tethered inhibitor by acting upon a substrate; or may be an inhibitory aptamer that inhibits an enzyme and prevents the enzyme from catalyzing generation of a detectable signal from a substrate or wherein the polynucleotide-tethered inhibitor inhibits an enzyme and prevents the enzyme from catalyzing generation of a detectable signal from a substrate; or sequesters a pair of agents that when released from the aptamers combine to generate a detectable signal.

The nanoparticle may be a colloidal metal. The colloidal metal material may include water-insoluble metal particles or metallic compounds dispersed in a liquid, a hydrosol, or a metal sol. The colloidal metal may be selected from the metals in groups IA, IB, IIB and IIIB of the periodic table, as well as the transition metals, especially those of group VIII. Preferred metals include gold, silver, aluminum, ruthenium, zinc, iron, nickel and calcium. Other suitable metals also include the following in all of their various oxidation states: lithium, sodium, magnesium, potassium, scandium, titanium, vanadium, chromium, manganese, cobalt, copper, gallium, strontium, niobium, molybdenum, palladium, indium, tin, tungsten, rhenium, platinum, and gadolinium. The metals are preferably provided in ionic form, derived from an appropriate metal compound, for example the A1³⁺, Ru³⁺, Zn²⁺, Fe³⁺, Ni²⁺ and Ca²⁺ ions.

When the RNA bridge is cut by the activated CRISPR effector, the beforementioned color shift is observed. In certain example embodiments the particles are colloidal metals. In certain other example embodiments, the colloidal metal is a colloidal gold. In certain example embodiments, the colloidal nanoparticles are 15 nm gold nanoparticles (AuNPs). Due to the unique surface properties of colloidal gold nanoparticles, maximal absorbance is observed at 520 nm when fully dispersed in solution and appear red in color to the naked eye. Upon aggregation of AuNPs, they exhibit a red-shift in maximal absorbance and appear darker in color, eventually precipitating from solution as a dark purple aggregate.

In some embodiments, at least one guide polynucleotide comprises a mismatch. The mismatch may be up- or downstream of a single nucleotide variation on the one or more guide sequences. In certain embodiments, modulations of cleavage efficiency can be exploited by introduction of mismatches, e.g. 1 or more mismatches, such as 1 or 2 mismatches between spacer sequence and target sequence, including the position of the mismatch along the spacer/target. The more central (i.e. not 3′ or 5′) for instance a double mismatch is, the more cleavage efficiency is affected. Accordingly, by choosing mismatch position along the spacer, cleavage efficiency can be modulated. By means of example, if less than 100% cleavage of targets is desired (e.g. in a cell population), 1 or more, such as preferably 2 mismatches between spacer and target sequence may be introduced in the spacer sequences. The more central along the spacer of the mismatch position, the lower the cleavage percentage. In certain example embodiments, the cleavage efficiency may be exploited to design single guides that can distinguish two or more targets that vary by a single nucleotide, such as a single nucleotide polymorphism (SNP), variation, or (point) mutation. The CRISPR effector may have reduced sensitivity to SNPs (or other single nucleotide variations) and continue to cleave SNP targets with a certain level of efficiency. Thus, for two targets, or a set of targets, a guide RNA may be designed with a nucleotide sequence that is complementary to one of the targets i.e. the on-target SNP. The guide RNA is further designed to have a synthetic mismatch. As used herein a “synthetic mismatch” refers to a non-naturally occurring mismatch that is introduced upstream or downstream of the naturally occurring SNP, such as at most 5 nucleotides upstream or downstream, for instance 4, 3, 2, or 1 nucleotide upstream or downstream, preferably at most 3 nucleotides upstream or downstream, more preferably at most 2 nucleotides upstream or downstream, most preferably 1 nucleotide upstream or downstream (i.e. adjacent the SNP). When the CRISPR effector binds to the on-target SNP, only a single mismatch will be formed with the synthetic mismatch and the CRISPR effector will continue to be activated and a detectable signal produced. When the guide RNA hybridizes to an off-target SNP, two mismatches will be formed, the mismatch from the SNP and the synthetic mismatch, and no detectable signal generated. Thus, the systems disclosed herein may be designed to distinguish SNPs within a population. For, example the systems may be used to distinguish pathogenic strains that differ by a single SNP or detect certain disease specific SNPs, such as but not limited to, disease associated SNPs, such as without limitation cancer associated SNPs.

In certain embodiments, the guide RNA is designed such that the SNP is located on position 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the spacer sequence (starting at the 5′ end). In certain embodiments, the guide RNA is designed such that the SNP is located on position 1, 2, 3, 4, 5, 6, 7, 8, or 9 of the spacer sequence (starting at the 5′ end). In certain embodiments, the guide RNA is designed such that the SNP is located on position 2, 3, 4, 5, 6, or 7 of the spacer sequence (starting at the 5′ end). In certain embodiments, the guide RNA is designed such that the SNP is located on position 3, 4, 5, or 6 of the spacer sequence (starting at the 5′ end). In certain embodiments, the guide RNA is designed such that the SNP is located on position 3 of the spacer sequence (starting at the 5′ end).

In certain embodiments, the guide RNA is designed such that the mismatch (e.g. The synthetic mismatch, i.e. an additional mutation besides a SNP) is located on position 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the spacer sequence (starting at the 5′ end). In certain embodiments, the guide RNA is designed such that the mismatch is located on position 1, 2, 3, 4, 5, 6, 7, 8, or 9 of the spacer sequence (starting at the 5′ end). In certain embodiments, the guide RNA is designed such that the mismatch is located on position 4, 5, 6, or 7 of the spacer sequence (starting at the 5′ end. In certain embodiments, the guide RNA is designed such that the mismatch is located on position 5 of the spacer sequence (starting at the 5′ end).

In certain embodiments, the guide RNA is designed such that the mismatch is located 2 nucleotides upstream of the SNP (i.e. one intervening nucleotide). In certain embodiments, the guide RNA is designed such that the mismatch is located 2 nucleotides downstream of the SNP (i.e. one intervening nucleotide). In certain embodiments, the guide RNA is designed such that the mismatch is located on position 5 of the spacer sequence (starting at the 5′ end) and the SNP is located on position 3 of the spacer sequence (starting at the 5′ end).

### Microbe Detection and Diagnostics

In an aspect, methods of diagnostics and/or detection comprise detecting the presence of one or more viruses or viral infections. Design of kits and systems for use in the diagnostic and detection methods are also provided. The virus may be a DNA virus, a RNA virus, or a retrovirus, or a combination thereof. Methods of viral detection are described in International Patent Publication No. WO 2018/170340. Particular viral applications include viruses as described in International Publication WO 2018/170340 at [0347]—[0354], and Tables 8 and 9, incorporated herein by reference. Viral diagnostics platforms can be developed utilizing the methods as described in Myrhvold et al., “Field Deployable viral diagnostics using CRISPR-Cas13” Science 360, 444-448 (2018). Clinical samples such as urine, plasma, saliva, whole blood, or serum can be used for sensitive detection of viral infections. Such methods can also be utilized with Cas proteins alone or in conjunction with Cas13 proteins, as described elsewhere herein.

Systems and methods can be designed for the detection and diagnosis of microbes, including bacterial, fungi and viral microbes. In an aspect, the systems may comprise multiplex detection of multiple variants of viral infections, including coronavirus, different viruses which may be related coronaviruses or respiratory viruses, or a combination thereof. In embodiments, assays can be performed for a variety of viruses and viral infections, including acute respiratory infections using the disclosure detailed herein. The systems can comprise two or more CRISPR Cas systems to multiplex, as described elsewhere herein, to detect a plurality of respiratory infections or viral infections, including coronavirus. The coronavirus is a positive-sense single stranded RNA family of viruses, infecting a variety of animals and humans. In one aspect, the detection systems are utilized to identify patients infected with a coronavirus, e.g., the 2019-nCoV, or a related coronavirus, for example, a coronavirus comprising at least 80%, 85%, 90%, 95%, 96%, 97%, 98% or 99% identity to the 2019-nCoV, GISAID deposit accession no. EPI_ISL_402124 and EPI_ISL_402127-402130, and described in DOI: 10.1101/2020.01.22.914952, or EP_ISL_402119-402121 and EP_ISL_402123-402124; see also GenBank Accession No. MN908947.3.

Examples of microbes (and infection thereof) that can be detected and/or diagnosed with the compositions, systems, and methods herein include those described in paragraphs [00288]-[00298] of Zhang et al., WO2019148206A1, which is incorporated by reference herein in its entirety.

### Transcript Tracking

In another aspect, the present disclosure provides compositions and methods for transcript tracking. In some embodiments, transcript tracking allows researchers to visualize transcripts in cells, tissues, organs or animals, providing important spatio-temporal information regarding RNA dynamics and function.

In some embodiments, the compositions may be a CRISPR-Cas protein herein with one or more labels, or a CRISPR-Cas system comprising such labeled CRISPR-Cas protein. The CRISPR-Cas protein or system may bind to one or more transcripts such that the transcripts may be detected (e.g., visualized) using the label on the CRISPR-Cas protein.

In some embodiments, the present disclosure includes a system for expressing a CRISPR-Cas protein with one or more polypeptides or polynucleotide labels. The system may comprise polynucleotides encoding the CRISPR-Cas protein and/or the labels. The system may further include vector systems comprising such polynucleotides. For example, a CRISPR-Cas protein may be fused with a fluorescent protein or a fragment thereof. Examples of fluorescent proteins include GFP proteins, EGFP, Azami-Green, Kaede, ZsGreen1 and CopGFP; CFP proteins, such as Cerulean, mCFP, AmCyan1, MiCy, and CyPet; BFP proteins such as EBFP; YFP proteins such as EYFP, YPet, Venus, ZsYellow, and mCitrine; OFP proteins such as cOFP, mKO, and mOrange; red fluorescent protein, or RFP; red or far-red fluorescent proteins from any other species, such as Heteractis reef coral and Actinia or Entacmaea sea anemone, as well as variants thereof. RFPs include, for example, Discosoma variants, such as mRFP1, mCherry, tdTomato, mStrawberry, mTangerine, DsRed2, and DsRed-T1, Anthomedusa J-Red and Anemonia AsRed2. Far-red fluorescent proteins include, for example, Actinia AQ143, Entacmaea eqFP611, Discosoma variants such as mPlum and mRasberry, and Heteractis HcRed1 and t-HcRed.

In some cases, the systems for expressing the labeled CRISPR-Cas protein may be inducible. For example, the systems may comprise polynucleotides encoding the CRISPR-Cas protein and/or labels under control of a regulatory element herein, e.g., inducible promoters. Such systems may allow spatial and/or temporal control of the expression of the labels, thus enabling spatial and/or temporal control of transcript tracking.

In certain cases, the CRISPR-Cas may be labeled with a detectable tag. The labeling may be performed in cells. Alternatively or additionally, the labeling may be performed first and the labeled CRISPR-Cas protein is then delivered into cells, tissues, organs, or organs.

The detectable tags may be detected (e.g., visualized by imaging, ultrasound, or MRI). Examples of such detectable tags include detectable oligonucleotide tags may be, but are not limited to, oligonucleotides comprising unique nucleotide sequences, oligonucleotides comprising detectable moieties, and oligonucleotides comprising both unique nucleotide sequences and detectable moieties. In some cases, the detectable tag comprises a labeling substance, which is detectable by spectroscopic, photochemical, biochemical, immunochemical, electrical, optical or chemical means. Such tags include biotin for staining with labeled streptavidin conjugate, magnetic beads (e.g., Dynabeads®), fluorescent dyes (e.g., fluorescein, texas red, rhodamine, green fluorescent protein, and the like), radiolabels (e.g., ³H, ¹²⁵I, ³⁵S, ¹⁴C, or 32P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Detectable tags may be detected by many methods. For example, radiolabels may be detected using photographic film or scintillation counters, fluorescent markers may be detected using a photodetector to detect emitted light. Enzymatic labels are typically detected by providing the enzyme with a substrate and detecting, the reaction product produced by the action of the enzyme on the substrate, and colorimetric labels are detected by simply visualizing the colored label. Examples of the labeling substance which may be employed include labeling substances known to those skilled in the art, such as fluorescent dyes, enzymes, coenzymes, chemiluminescent substances, and radioactive substances. Specific examples include radioisotopes (e.g., ³²p, ¹⁴C, ¹²⁵I, ³H, and ¹³¹I), fluorescein, rhodamine, dansyl chloride, umbelliferone, luciferase, peroxidase, alkaline phosphatase, β-galactosidase, β-glucosidase, horseradish peroxidase, glucoamylase, lysozyme, saccharide oxidase, microperoxidase, biotin, and ruthenium. In the case where biotin is employed as a labeling substance, preferably, after addition of a biotin-labeled antibody, streptavidin bound to an enzyme (e.g., peroxidase) is further added. Advantageously, the label is a fluorescent label. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. A fluorescent label may be a fluorescent protein, such as blue fluorescent protein, cyan fluorescent protein, green fluorescent protein, red fluorescent protein, yellow fluorescent protein or any photoconvertible protein. Colorimetric labeling, bioluminescent labeling and/or chemiluminescent labeling may further accomplish labeling. Labeling further may include energy transfer between molecules in the hybridization complex by perturbation analysis, quenching, or electron transport between donor and acceptor molecules, the latter of which may be facilitated by double stranded match hybridization complexes. The fluorescent label may be a perylene or a terrylen. In the alternative, the fluorescent label may be a fluorescent bar code. Advantageously, the label may be light sensitive, wherein the label is light-activated and/or light cleaves the one or more linkers to release the molecular cargo. The light-activated molecular cargo may be a major light-harvesting complex (LHCII). In another embodiment, the fluorescent label may induce free radical formation. In some embodiments, the detectable moieties may be quantum dots.

In some embodiments, the present disclosure provides for a system for delivery the labeled CRISPR-Cas proteins or labeled CRISPR-Cas systems. The delivery system may comprise any delivery vehicles, e.g., those described herein such as RNP, liposomes, nanoparticles, exosomes, microvesicles, nucleic acid nanoassemblies, a gene gun, an implantable device, or the vector systems herein.

### Nucleic Acid Targeting

In certain embodiments, the CRISPR-Cas effector protein of the invention is, or in, or comprises, or consists essentially of, or consists of, or involves or relates to such a protein from or as set forth herein, wherein one or more amino acids are mutated, as described herein elsewhere. Thus, in some embodiments, the effector protein may be a RNA-binding protein, such as a dead-Cas type effector protein, which may be optionally functionalized as described herein for instance with an transcriptional activator or repressor domain, NLS or other functional domain. In some embodiments, the effector protein may be a RNA-binding protein that cleaves a single strand of RNA. If the RNA bound is ssRNA, then the ssRNA is fully cleaved. In some embodiments, the effector protein may be a RNA-binding protein that cleaves a double strand of RNA, for example if it comprises two RNase domains. If the RNA bound is dsRNA, then the dsRNA is fully cleaved. In some embodiments, the effector protein may be a RNA-binding protein that has nickase activity, i.e. it binds dsRNA, but only cleaves one of the RNA strands.

RNase function in CRISPR systems is known, for example mRNA targeting has been reported for certain type III CRISPR-Cas systems (Hale et al., 2014, Genes Dev, vol. 28, 2432-2443; Hale et al., 2009, Cell, vol. 139, 945-956; Peng et al., 2015, Nucleic acids research, vol. 43, 406-417) and provides significant advantages. A CRISPR-Cas system, composition or method targeting RNA via the present effector proteins is thus provided.

The target RNA, i.e. the RNA of interest, is the RNA to be targeted by the present invention leading to the recruitment to, and the binding of the effector protein at, the target site of interest on the target RNA. The target RNA may be any suitable form of RNA. This may include, in some embodiments, mRNA. In other embodiments, the target RNA may include tRNA or rRNA.

### Self-Inactivating Systems

Once all copies of RNA in a cell have been edited, continued a CRISPR-Cas effector protein expression or activity in that cell is no longer necessary. A Self-Inactivating system that relies on the use of RNA as to the CRISPR-Cas or crRNA as the guide target sequence can shut down the system by preventing expression of CRISPR-Cas or complex formation.

### Examples of Target RNAs

The compositions and systems herein may be used for modifying various types of target RNAs. In some embodiments, the compositions and systems may be used to modify the target RNAs in a sequence-specific manner. For example, the target RNAs comprise target sequences for the guide sequences. Alternatively or additionally, in some embodiments, the compositions and systems may be used to modify the target RNAs in a non-sequence-specific manner. For example, the target RNAs may be modified or cleaved by the collateral activity of the Cas protein in the compositions and systems. Examples of target RNAs include those described below.

Interfering RNA (RNAi) and microRNA (miRNA)

In other embodiments, the target RNA may include interfering RNA, i.e. RNA involved in an RNA interference pathway, such as shRNA, siRNA and so forth. In other embodiments, the target RNA may include microRNA (miRNA). Control over interfering RNA or miRNA may help reduce off-target effects (OTE) seen with those approaches by reducing the longevity of the interfering RNA or miRNA in vivo or in vitro.

If the effector protein and suitable guide are selectively expressed (for example spatially or temporally under the control of a suitable promoter, for example a tissue- or cell cycle-specific promoter and/or enhancer) then this could be used to ‘protect’ the cells or systems (in vivo or in vitro) from RNAi in those cells. This may be useful in neighboring tissues or cells where RNAi is not required or for the purposes of comparison of the cells or tissues where the effector protein and suitable guide are and are not expressed (i.e. where the RNAi is not controlled and where it is, respectively). The effector protein may be used to control or bind to molecules comprising or consisting of RNA, such as ribozymes, ribosomes or riboswitches. In embodiments of the invention, the RNA guide can recruit the effector protein to these molecules so that the effector protein is able to bind to them.

Ribosomal RNA (rRNA)

For example, azalide antibiotics such as azithromycin, are well known. They target and disrupt the 50S ribosomal subunit. The present effector protein, together with a suitable guide RNA to target the 50S ribosomal subunit, may be, in some embodiments, recruited to and bind to the 50S ribosomal subunit. Thus, the present effector protein in concert with a suitable guide directed at a ribosomal (especially the 50 s ribosomal subunit) target is provided. Use of this use effector protein in concert with the suitable guide directed at the ribosomal (especially the 50 s ribosomal subunit) target may include antibiotic use. In particular, the antibiotic use is analogous to the action of azalide antibiotics, such as azithromycin. In some embodiments, prokaryotic ribosomal subunits, such as the 70S subunit in prokaryotes, the 50S subunit mentioned above, the 30S subunit, as well as the 16S and 5S subunits may be targeted. In other embodiments, eukaryotic ribosomal subunits, such as the 80S subunit in eukaryotes, the 60S subunit, the 40S subunit, as well as the 28S, 18S. 5.8S and 5S subunits may be targeted.

The effector protein may be a RNA-binding protein, optionally functionalized, as described herein. In some embodiments, the effector protein may be a RNA-binding protein that cleaves a single strand of RNA. In either case, but particularly where the RNA-binding protein cleaves a single strand of RNA, then ribosomal function may be modulated and, in particular, reduced or destroyed. This may apply to any ribosomal RNA and any ribosomal subunit and the sequences of rRNA are well known.

Control of ribosomal activity is thus envisaged through use of the present effector protein in concert with a suitable guide to the ribosomal target. This may be through cleavage of, or binding to, the ribosome. In particular, reduction of ribosomal activity is envisaged. This may be useful in assaying ribosomal function in vivo or in vitro, but also as a means of controlling therapies based on ribosomal activity, in vivo or in vitro. Furthermore, control (i.e. reduction) of protein synthesis in an in vivo or in vitro system is envisaged, such control including antibiotic and research and diagnostic use.

### Riboswitches

A riboswitch (also known as an aptozyme) is a regulatory segment of a messenger RNA molecule that binds a small molecule. This typically results in a change in production of the proteins encoded by the mRNA. Thus, control of riboswitch activity is thus envisaged through use of the present effector protein in concert with a suitable guide to the riboswitch target. This may be through cleavage of, or binding to, the riboswitch. In particular, reduction of riboswitch activity is envisaged. This may be useful in assaying riboswitch function in vivo or in vitro, but also as a means of controlling therapies based on riboswitch activity, in vivo or in vitro. Furthermore, control (i.e. reduction) of protein synthesis in an in vivo or in vitro system is envisaged. This control, as for rRNA may include antibiotic and research and diagnostic use.

### Ribozymes

Ribozymes are RNA molecules having catalytic properties, analogous to enzymes (which are of course proteins). As ribozymes, both naturally occurring and engineered, comprise or consist of RNA, they may also be targeted by the present RNA-binding effector protein. In some embodiments, the effector protein may be a RNA-binding protein cleaves the ribozyme to thereby disable it. Control of ribozymal activity is thus envisaged through use of the present effector protein in concert with a suitable guide to the ribozymal target. This may be through cleavage of, or binding to, the ribozyme. In particular, reduction of ribozymal activity is envisaged. This may be useful in assaying ribozymal function in vivo or in vitro, but also as a means of controlling therapies based on ribozymal activity, in vivo or in vitro.

### RNA-Targeting Applications

### Gene Expression, Including RNA Processing

The effector protein may also be used, together with a suitable guide, to target gene expression, including via control of RNA processing. The control of RNA processing may include RNA processing reactions such as RNA splicing, including alternative splicing, via targeting of RNApol; viral replication (in particular of satellite viruses, bacteriophages and retroviruses, such as HBV, HBC and HIV and others listed herein) including virioids in plants; and tRNA biosynthesis. The effector protein and suitable guide may also be used to control RNA activation (RNAa). RNAa leads to the promotion of gene expression, so control of gene expression may be achieved that way through disruption or reduction of RNAa and thus less promotion of gene expression.

### RNAi Screens

Identifying gene products whose knockdown is associated with phenotypic changes, biological pathways can be interrogated and the constituent parts identified, via RNAi screens. Control may also be exerted over or during these screens by use of the effector protein and suitable guide to remove or reduce the activity of the RNAi in the screen and thus reinstate the activity of the (previously interfered with) gene product (by removing or reducing the interference/repression).

Satellite RNAs (satRNAs) and satellite viruses may also be treated.

Control herein with reference to RNase activity generally means reduction, negative disruption or known-down or knock out.

### In Vivo RNA Applications

### Inhibition of Gene Expression

The target-specific RNases provided herein allow for very specific cutting of a target RNA. The interference at RNA level allows for modulation both spatially and temporally and in a non-invasive way, as the genome is not modified.

A number of diseases have been demonstrated to be treatable by mRNA targeting. While most of these studies relate to administration of siRNA, it is clear that the RNA targeting effector proteins provided herein can be applied in a similar way.

Examples of mRNA targets (and corresponding disease treatments) are VEGF, VEGF-R1 and RTP801 (in the treatment of AMD and/or DME), Caspase 2 (in the treatment of Naion) ADRB2 (in the treatment of intraocular pressure), TRPVI (in the treatment of Dry eye syndrome, Syk kinase (in the treatment of asthma), Apo B (in the treatment of hypercholesterolemia), PLK1, KSP and VEGF (in the treatment of solid tumors), Ber-Abl (in the treatment of CML)(Burnett and Rossi Chem Biol. 2012, 19(1): 60-71)). Similarly, RNA targeting has been demonstrated to be effective in the treatment of RNA-virus mediated diseases such as HIV (targeting of HIV Tet and Rev), RSV (targeting of RSV nucleocapsid) and HCV (targeting of miR-122) (Burnett and Rossi Chem Biol. 2012, 19(1): 60-71).

It is further envisaged that the RNA targeting effector protein of the invention can be used for mutation specific or allele specific knockdown. Guide RNA's can be designed that specifically target a sequence in the transcribed mRNA comprising a mutation or an allele-specific sequence. Such specific knockdown is particularly suitable for therapeutic applications relating to disorders associated with mutated or allele-specific gene products. For example, most cases of familial hypobetalipoproteinemia (FHBL) are caused by mutations in the ApoB gene. This gene encodes two versions of the apolipoprotein B protein: a short version (ApoB-48) and a longer version (ApoB-100). Several ApoB gene mutations that lead to FHBL cause both versions of ApoB to be abnormally short. Specifically targeting and knockdown of mutated ApoB mRNA transcripts with an RNA targeting effector protein of the invention may be beneficial in treatment of FHBL. As another example, Huntington's disease (HD) is caused by an expansion of CAG triplet repeats in the gene coding for the Huntingtin protein, which results in an abnormal protein. Specifically targeting and knockdown of mutated or allele-specific mRNA transcripts encoding the Huntingtin protein with an RNA targeting effector protein of the invention may be beneficial in treatment of HD.

### Modulation of Gene Expression Through Modulation of RNA Function

Apart from a direct effect on gene expression through cleavage of the mRNA, RNA targeting can also be used to impact specific aspects of the RNA processing within the cell, which may allow a more subtle modulation of gene expression. Generally, modulation can for instance be mediated by interfering with binding of proteins to the RNA, such as for instance blocking binding of proteins, or recruiting RNA binding proteins. Indeed, modulations can be ensured at different levels such as splicing, transport, localization, translation and turnover of the mRNA. Similarly in the context of therapy, it can be envisaged to address (pathogenic) malfunctioning at each of these levels by using RNA-specific targeting molecules. In these embodiments it is in many cases preferred that the RNA targeting protein is a “dead” CRISPR-Cas that has lost the ability to cut the RNA target but maintains its ability to bind thereto, such as the mutated forms of CRISPR-Cas described herein.

### a) Alternative Splicing

Many of the human genes express multiple mRNAs as a result of alternative splicing. Different diseases have been shown to be linked to aberrant splicing leading to loss of function or gain of function of the expressed gene. While some of these diseases are caused by mutations that cause splicing defects, a number of these are not. One therapeutic option is to target the splicing mechanism directly. The RNA targeting effector proteins described herein can for instance be used to block or promote slicing, include or exclude exons and influence the expression of specific isoforms and/or stimulate the expression of alternative protein products. Such applications are described in more detail below.

A RNA targeting effector protein binding to a target RNA can sterically block access of splicing factors to the RNA sequence. The RNA targeting effector protein targeted to a splice site may block splicing at the site, optionally redirecting splicing to an adjacent site. For instance a RNA targeting effector protein binding to the 5′ splice site binding can block the recruitment of the U1 component of the spliceosome, favoring the skipping of that exon. Alternatively, a RNA targeting effector protein targeted to a splicing enhancer or silencer can prevent binding of transacting regulatory splicing factors at the target site and effectively block or promote splicing. Exon exclusion can further be achieved by recruitment of ILF2/3 to precursor mRNA near an exon by an RNA targeting effector protein as described herein. As yet another example, a glycine rich domain can be attached for recruitment of hnRNP A1 and exon exclusion (Del Gatto-Konczak et al. Mol Cell Biol. 1999 January; 19(l):251-60).

In certain embodiments, through appropriate selection of gRNA, specific splice variants may be targeted, while other splice variants will not be targeted

In some cases the RNA targeting effector protein can be used to promote slicing (e.g. where splicing is defective). For instance a RNA targeting effector protein can be associated with an effector capable of stabilizing a splicing regulatory stem-loop in order to further splicing. The RNA targeting effector protein can be linked to a consensus binding site sequence for a specific splicing factor in order to recruit the protein to the target DNA.

Examples of diseases which have been associated with aberrant splicing include, but are not limited to Paraneoplastic Opsoclonus Myoclonus Ataxia (or POMA), resulting from a loss of Nova proteins which regulate splicing of proteins that function in the synapse, and Cystic Fibrosis, which is caused by defective splicing of a cystic fibrosis transmembrane conductance regulator, resulting in the production of nonfunctional chloride channels. In other diseases aberrant RNA splicing results in gain-of-function. This is the case for instance in myotonic dystrophy which is caused by a CUG triplet-repeat expansion (from 50 to >1500 repeats) in the 3′UTR of an mRNA, causing splicing defects.

The RNA targeting effector protein can be used to include an exon by recruiting a splicing factor (such as U1) to a 5′ splicing site to promote excision of introns around a desired exon. Such recruitment could be mediated trough a fusion with an arginine/serine rich domain, which functions as splicing activator (Gravely B R and Maniatis T, Mol Cell. 1998 (5):765-71).

It is envisaged that the RNA targeting effector protein can be used to block the splicing machinery at a desired locus, resulting in preventing exon recognition and the expression of a different protein product. An example of a disorder that may treated is Duchenne muscular dystrophy (DMD), which is caused by mutations in the gene encoding for the dystrophin protein. Almost all DMD mutations lead to frameshifts, resulting in impaired dystrophin translation. The RNA targeting effector protein can be paired with splice junctions or exonic splicing enhancers (ESEs) thereby preventing exon recognition, resulting in the translation of a partially functional protein. This converts the lethal Duchenne phenotype into the less severe Becker phenotype.

### b) RNA Modification

RNA editing is a natural process whereby the diversity of gene products of a given sequence is increased by minor modification in the RNA. Typically, the modification involves the conversion of adenosine (A) to inosine (I), resulting in an RNA sequence which is different from that encoded by the genome. RNA modification is generally ensured by the ADAR enzyme, whereby the pre-RNA target forms an imperfect duplex RNA by base-pairing between the exon that contains the adenosine to be edited and an intronic non-coding element. A classic example of A-I editing is the glutamate receptor GluR-B mRNA, whereby the change results in modified conductance properties of the channel (Higuchi M, et al. Cell. 1993; 75:1361-70).

In humans, a heterozygous functional-null mutation in the ADAR1 gene leads to a skin disease, human pigmentary genodermatosis (Miyamura Y, et al. Am J Hum Genet. 2003; 73:693-9). It is envisaged that the RNA targeting effector proteins of the present invention can be used to correct malfunctioning RNA modification.

### c) Polyadenylation

Polyadenylation of an mRNA is important for nuclear transport, translation efficiency and stability of the mRNA, and all of these, as well as the process of polyadenylation, depend on specific RBPs. Most eukaryotic mRNAs receive a 3′ poly(A) tail of about 200 nucleotides after transcription. Polyadenylation involves different RNA-binding protein complexes which stimulate the activity of a poly(A)polymerase (Minvielle-Sebastia L et al. Curr Opin Cell Biol. 1999; 11:352-7). It is envisaged that the RNA-targeting effector proteins provided herein can be used to interfere with or promote the interaction between the RNA-binding proteins and RNA.

Examples of diseases which have been linked to defective proteins involved in polyadenylation are oculopharyngeal muscular dystrophy (OPMD) (Brais B, et al. Nat Genet. 1998; 18:164-7).

### d) RNA Export

After pre-mRNA processing, the mRNA is exported from the nucleus to the cytoplasm. This is ensured by a cellular mechanism which involves the generation of a carrier complex, which is then translocated through the nuclear pore and releases the mRNA in the cytoplasm, with subsequent recycling of the carrier.

Overexpression of proteins (such as TAP) which play a role in the export of RNA has been found to increase export of transcripts that are otherwise inefficiently exported in Xenopus (Katahira J, et al. EMBO J. 1999; 18:2593-609).

e) mRNA Localization

mRNA localization ensures spatially regulated protein production. Localization of transcripts to a specific region of the cell can be ensured by localization elements. In particular embodiments, it is envisaged that the effector proteins described herein can be used to target localization elements to the RNA of interest. The effector proteins can be designed to bind the target transcript and shuttle them to a location in the cell determined by its peptide signal tag. More particularly for instance, a RNA targeting effector protein fused to a nuclear localization signal (NLS) can be used to alter RNA localization.

Further examples of localization signals include the zipcode binding protein (ZBP1) which ensures localization of β-actin to the cytoplasm in several asymmetric cell types, KDEL retention sequence (localization to endoplasmic reticulum), nuclear export signal (localization to cytoplasm), mitochondrial targeting signal (localization to mitochondria), peroxisomal targeting signal (localization to peroxisome) and m6A marking/YTHDF2 (localization to p-bodies). Other approaches that are envisaged are fusion of the RNA targeting effector protein with proteins of known localization (for instance membrane, synapse).

Alternatively, the effector protein according to the invention may for instance be used in localization-dependent knockdown. By fusing the effector protein to an appropriate localization signal, the effector is targeted to a particular cellular compartment. Only target RNAs residing in this compartment will effectively be targeted, whereas otherwise identical targets, but residing in a different cellular compartment will not be targeted, such that a localization dependent knockdown can be established.

### f) Translation

The RNA targeting effector proteins described herein can be used to enhance or repress translation. It is envisaged that upregulating translation is a very robust way to control cellular circuits. Further, for functional studies a protein translation screen can be favorable over transcriptional upregulation screens, which have the shortcoming that upregulation of transcript does not translate into increased protein production.

It is envisaged that the RNA targeting effector proteins described herein can be used to bring translation initiation factors, such as EIF4G in the vicinity of the 5′ untranslated repeat (5′UTR) of a messenger RNA of interest to drive translation (as described in De Gregorio et al. EMBO J. 1999; 18(17):4865-74 for a non-reprogrammable RNA binding protein). As another example GLD2, a cytoplasmic poly(A) polymerase, can be recruited to the target mRNA by an RNA targeting effector protein. This would allow for directed polyadenylation of the target mRNA thereby stimulating translation.

Similarly, the RNA targeting effector proteins envisaged herein can be used to block translational repressors of mRNA, such as ZBP1 (Huttelmaier S, et al. Nature. 2005; 438:512-5). By binding to translation initiation site of a target RNA, translation can be directly affected.

In addition, fusing the RNA targeting effector proteins to a protein that stabilizes mRNAs, e.g. by preventing degradation thereof such as RNase inhibitors, it is possible to increase protein production from the transcripts of interest.

It is envisaged that the RNA targeting effector proteins described herein can be used to repress translation by binding in the 5′ UTR regions of a RNA transcript and preventing the ribosome from forming and beginning translation.

Further, the RNA targeting effector protein can be used to recruit Caf1, a component of the CCR4-NOT deadenylase complex, to the target mRNA, resulting in deadenylation or the target transcript and inhibition of protein translation.

For instance, the RNA targeting effector protein of the invention can be used to increase or decrease translation of therapeutically relevant proteins. Examples of therapeutic applications wherein the RNA targeting effector protein can be used to downregulate or upregulate translation are in amyotrophic lateral sclerosis (ALS) and cardiovascular disorders. Reduced levels of the glial glutamate transporter EAAT2 have been reported in ALS motor cortex and spinal cord, as well as multiple abnormal EAAT2 mRNA transcripts in ALS brain tissue. Loss of the EAAT2 protein and function thought to be the main cause of excitotoxicity in ALS. Restoration of EAAT2 protein levels and function may provide therapeutic benefit. Hence, the RNA targeting effector protein can be beneficially used to upregulate the expression of EAAT2 protein, e.g. by blocking translational repressors or stabilizing mRNA as described above. Apolipoprotein A1 is the major protein component of high density lipoprotein (HDL) and ApoA1 and HDL are generally considered as atheroprotective. It is envisaged that the RNA targeting effector protein can be beneficially used to upregulate the expression of ApoA1, e.g. by blocking translational repressors or stabilizing mRNA as described above.

g) mRNA Turnover

Translation is tightly coupled to mRNA turnover and regulated mRNA stability. Specific proteins have been described to be involved in the stability of transcripts (such as the ELAV/Hu proteins in neurons, Keene J D, 1999, Proc Natl Acad Sci USA. 96:5-7) and tristetraprolin (TTP). These proteins stabilize target mRNAs by protecting the messages from degradation in the cytoplasm (Peng S. S. et al., 1988, EMBO J. 17:3461-70).

It can be envisaged that the RNA-targeting effector proteins of the present invention can be used to interfere with or to promote the activity of proteins acting to stabilize mRNA transcripts, such that mRNA turnover is affected. For instance, recruitment of human TTP to the target RNA using the RNA targeting effector protein would allow for adenylate-uridylate-rich element (AU-rich element) mediated translational repression and target degradation. AU-rich elements are found in the 3′ UTR of many mRNAs that code for proto-oncogenes, nuclear transcription factors, and cytokines and promote RNA stability. As another example, the RNA targeting effector protein can be fused to HuR, another mRNA stabilization protein (Hinman M N and Lou H, Cell Mol Life Sci 2008; 65:3168-81), and recruit it to a target transcript to prolong its lifetime or stabilize short-lived mRNA.

It is further envisaged that the RNA-targeting effector proteins described herein can be used to promote degradation of target transcripts. For instance, m6A methyltransferase can be recruited to the target transcript to localize the transcript to β-bodies leading to degradation of the target.

As yet another example, an RNA targeting effector protein as described herein can be fused to the non-specific endonuclease domain PilT N-terminus (PIN), to recruit it to a target transcript and allow degradation thereof.

Patients with paraneoplastic neurological disorder (PND)-associated encephalomyelitis and neuropathy are patients who develop autoantibodies against Hu-proteins in tumors outside of the central nervous system (Szabo A et al. 1991, Cell.; 67:325-33 which then cross the blood-brain barrier. It can be envisaged that the RNA-targeting effector proteins of the present invention can be used to interfere with the binding of auto-antibodies to mRNA transcripts.

Patients with dystrophy type 1 (DM1), caused by the expansion of (CUG)n in the 3′ UTR of dystrophia myotonica-protein kinase (DMPK) gene, are characterized by the accumulation of such transcripts in the nucleus. It is envisaged that the RNA targeting effector proteins of the invention fused with an endonuclease targeted to the (CUG)n repeats could inhibit such accumulation of aberrant transcripts.

h) Interaction with Multi-Functional Proteins

Some RNA-binding proteins bind to multiple sites on numerous RNAs to function in diverse processes. For instance, the hnRNP A1 protein has been found to bind exonic splicing silencer sequences, antagonizing the splicing factors, associate with telomere ends (thereby stimulating telomere activity) and bind miRNA to facilitate Drosha-mediated processing thereby affecting maturation. It is envisaged that the RNA-binding effector proteins of the present invention can interfere with the binding of RNA-binding proteins at one or more locations.

### i) RNA Folding

RNA adopts a defined structure in order to perform its biological activities. Transitions in conformation among alternative tertiary structures are critical to most RNA-mediated processes. However, RNA folding can be associated with several problems. For instance, RNA may have a tendency to fold into, and be upheld in, improper alternative conformations and/or the correct tertiary structure may not be sufficiently thermodynamically favored over alternative structures. The RNA targeting effector protein, in particular a cleavage-deficient or dead RNA targeting protein, of the invention may be used to direct folding of (m)RNA and/or capture the correct tertiary structure thereof.

### Modulation of Cellular Status

In certain embodiments CRISPR-Cas in a complex with crRNA is activated upon binding to target RNA and subsequently cleaves any nearby ssRNA targets (i.e. “collateral” or “bystander” effects). CRISPR-Cas, once primed by the cognate target, can cleave other (non-complementary) RNA molecules. Such promiscuous RNA cleavage could potentially cause cellular toxicity, or otherwise affect cellular physiology or cell status.

Accordingly, in certain embodiments, the non-naturally occurring or engineered composition, vector system, or delivery systems as described herein are used for or are for use in induction of cell dormancy. In certain embodiments, the non-naturally occurring or engineered composition, vector system, or delivery systems as described herein are used for or are for use in induction of cell cycle arrest. In certain embodiments, the non-naturally occurring or engineered composition, vector system, or delivery systems as described herein are used for or are for use in reduction of cell growth and/or cell proliferation, In certain embodiments, the non-naturally occurring or engineered composition, vector system, or delivery systems as described herein are used for or are for use in induction of cell anergy. In certain embodiments, the non-naturally occurring or engineered composition, vector system, or delivery systems as described herein are used for or are for use in induction of cell apoptosis. In certain embodiments, the non-naturally occurring or engineered composition, vector system, or delivery systems as described herein are used for or are for use in induction of cell necrosis. In certain embodiments, the non-naturally occurring or engineered composition, vector system, or delivery systems as described herein are used for or are for use in induction of cell death. In certain embodiments, the non-naturally occurring or engineered composition, vector system, or delivery systems as described herein are used for or are for use in induction of programmed cell death.

In certain embodiments, the invention relates to a method for induction of cell dormancy comprising introducing or inducing the non-naturally occurring or engineered composition, vector system, or delivery systems as described herein. In certain embodiments, the invention relates to a method for induction of cell cycle arrest comprising introducing or inducing the non-naturally occurring or engineered composition, vector system, or delivery systems as described herein. In certain embodiments, the invention relates to a method for reduction of cell growth and/or cell proliferation comprising introducing or inducing the non-naturally occurring or engineered composition, vector system, or delivery systems as described herein. In certain embodiments, the invention relates to a method for induction of cell anergy comprising introducing or inducing the non-naturally occurring or engineered composition, vector system, or delivery systems as described herein. In certain embodiments, the invention relates to a method for induction of cell apoptosis comprising introducing or inducing the non-naturally occurring or engineered composition, vector system, or delivery systems as described herein. In certain embodiments, the invention relates to a method for induction of cell necrosis comprising introducing or inducing the non-naturally occurring or engineered composition, vector system, or delivery systems as described herein. In certain embodiments, the invention relates to a method for induction of cell death comprising introducing or inducing the non-naturally occurring or engineered composition, vector system, or delivery systems as described herein. In certain embodiments, the invention relates to a method for induction of programmed cell death comprising introducing or inducing the non-naturally occurring or engineered composition, vector system, or delivery systems as described herein.

The methods and uses as described herein may be therapeutic or prophylactic and may target particular cells, cell (sub)populations, or cell/tissue types. In particular, the methods and uses as described herein may be therapeutic or prophylactic and may target particular cells, cell (sub)populations, or cell/tissue types expressing one or more target sequences, such as one or more particular target RNA (e.g. ss RNA). Without limitation, target cells may for instance be cancer cells expressing a particular transcript, e.g. neurons of a given class, (immune) cells causing e.g. autoimmunity, or cells infected by a specific (e.g. viral) pathogen, etc.

Accordingly, in certain embodiments, the invention relates to a method for treating a pathological condition characterized by the presence of undesirable cells (host cells), comprising introducing or inducing the non-naturally occurring or engineered composition, vector system, or delivery systems as described herein. In certain embodiments, the invention relates the use of the non-naturally occurring or engineered composition, vector system, or delivery systems as described herein for treating a pathological condition characterized by the presence of undesirable cells (host cells). In certain embodiments, the invention relates the non-naturally occurring or engineered composition, vector system, or delivery systems as described herein for use in treating a pathological condition characterized by the presence of undesirable cells (host cells). It is to be understood that preferably the CRISPR-Cas system targets a target specific for the undesirable cells. In certain embodiments, the invention relates to the use of the non-naturally occurring or engineered composition, vector system, or delivery systems as described herein for treating, preventing, or alleviating cancer. In certain embodiments, the invention relates to the non-naturally occurring or engineered composition, vector system, or delivery systems as described herein for use in treating, preventing, or alleviating cancer. In certain embodiments, the invention relates to a method for treating, preventing, or alleviating cancer comprising introducing or inducing the non-naturally occurring or engineered composition, vector system, or delivery systems as described herein. It is to be understood that preferably the CRISPR-Cas system targets a target specific for the cancer cells. In certain embodiments, the invention relates to the use of the non-naturally occurring or engineered composition, vector system, or delivery systems as described herein for treating, preventing, or alleviating infection of cells by a pathogen. In certain embodiments, the invention relates to the non-naturally occurring or engineered composition, vector system, or delivery systems as described herein for use in treating, preventing, or alleviating infection of cells by a pathogen. In certain embodiments, the invention relates to a method for treating, preventing, or alleviating infection of cells by a pathogen comprising introducing or inducing the non-naturally occurring or engineered composition, vector system, or delivery systems as described herein. It is to be understood that preferably the CRISPR-Cas system targets a target specific for the cells infected by the pathogen (e.g. a pathogen derived target). In certain embodiments, the invention relates to the use of the non-naturally occurring or engineered composition, vector system, or delivery systems as described herein for treating, preventing, or alleviating an autoimmune disorder. In certain embodiments, the invention relates to the non-naturally occurring or engineered composition, vector system, or delivery systems as described herein for use in treating, preventing, or alleviating an autoimmune disorder. In certain embodiments, the invention relates to a method for treating, preventing, or alleviating an autoimmune disorder comprising introducing or inducing the non-naturally occurring or engineered composition, vector system, or delivery systems as described herein. It is to be understood that preferably the CRISPR-Cas system targets a target specific for the cells responsible for the autoimmune disorder (e.g. specific immune cells).

### RNA Detection

It is further envisaged that the RNA targeting effector protein can be used in Northern blot assays. Northern blotting involves the use of electrophoresis to separate RNA samples by size. The RNA targeting effector protein can be used to specifically bind and detect the target RNA sequence.

A RNA targeting effector protein can be fused to a fluorescent protein (such as GFP) and used to track RNA localization in living cells. More particularly, the RNA targeting effector protein can be inactivated in that it no longer cleaves RNA. In particular embodiments, it is envisaged that a split RNA targeting effector protein can be used, whereby the signal is dependent on the binding of both subproteins, in order to ensure a more precise visualization. Alternatively, a split fluorescent protein can be used that is reconstituted when multiple RNA targeting effector protein complexes bind to the target transcript. It is further envisaged that a transcript is targeted at multiple binding sites along the mRNA so the fluorescent signal can amplify the true signal and allow for focal identification. As yet another alternative, the fluorescent protein can be reconstituted form a split intein.

RNA targeting effector proteins are for instance suitably used to determine the localization of the RNA or specific splice variants, the level of mRNA transcript, up- or down-regulation of transcripts and disease-specific diagnosis. The RNA targeting effector proteins can be used for visualization of RNA in (living) cells using e.g. fluorescent microscopy or flow cytometry, such as fluorescence-activated cell sorting (FACS) which allows for high-throughput screening of cells and recovery of living cells following cell sorting. Further, expression levels of different transcripts can be assessed simultaneously under stress, e.g. inhibition of cancer growth using molecular inhibitors or hypoxic conditions on cells. Another application would be to track localization of transcripts to synaptic connections during a neural stimulus using two photon microscopy.

In certain embodiments, the components or complexes according to the invention as described herein can be used in multiplexed error-robust fluorescence in situ hybridization (MERFISH; Chen et al. Science; 2015; 348(6233)), such as for instance with (fluorescently) labeled CRISPR-Cas effectors.

### In Vitro Apex Labeling

Cellular processes depend on a network of molecular interactions among protein, RNA, and DNA. Accurate detection of protein-DNA and protein-RNA interactions is key to understanding such processes. In vitro proximity labeling technology employs an affinity tag combined with e.g. a photoactivatable probe to label polypeptides and RNAs in the vicinity of a protein or RNA of interest in vitro. After UV irradiation the photoactivatable group reacts with proteins and other molecules that are in close proximity to the tagged molecule, thereby labelling them. Labelled interacting molecules can subsequently be recovered and identified. The RNA targeting effector protein of the invention can for instance be used to target a probe to a selected RNA sequence.

These applications could also be applied in animal models for in vivo imaging of disease relevant applications or difficult-to culture cell types.

### RNA Origami/In Vitro Assembly Lines—Combinatorics

RNA origami refers to nanoscale folded structures for creating two-dimensional or three-dimensional structures using RNA as integrated template. The folded structure is encoded in the RNA and the shape of the resulting RNA is thus determined by the synthesized RNA sequence (Geary, et al. 2014. Science, 345 (6198). pp. 799-804). The RNA origami may act as scaffold for arranging other components, such as proteins, into complexes. The RNA targeting effector protein of the invention can for instance be used to target proteins of interest to the RNA origami using a suitable guide RNA.

These applications could also be applied in animal models for in vivo imaging of disease relevant applications or difficult-to culture cell types.

### RNA ISOLATION OR PURIFICATION, ENRICHMENT OR DEPLETION

It is further envisaging that the RNA targeting effector protein when complexed to RNA can be used to isolate and/or purify the RNA. The RNA targeting effector protein can for instance be fused to an affinity tag that can be used to isolate and/or purify the RNA-RNA targeting effector protein complex. Such applications are for instance useful in the analysis of gene expression profiles in cells.

In particular embodiments, it can be envisaged that the RNA targeting effector proteins can be used to target a specific noncoding RNA (ncRNA) thereby blocking its activity, providing a useful functional probe. In certain embodiments, the effector protein as described herein may be used to specifically enrich for a particular RNA (including but not limited to increasing stability, etc.), or alternatively to specifically deplete a particular RNA (such as without limitation for instance particular splice variants, isoforms, etc.).

### Interrogation of LincRNA Function and Other Nuclear RNAs

Current RNA knockdown strategies such as siRNA have the disadvantage that they are mostly limited to targeting cytosolic transcripts since the protein machinery is cytosolic. The advantage of a RNA targeting effector protein of the present invention, an exogenous system that is not essential to cell function, is that it can be used in any compartment in the cell. By fusing a NLS signal to the RNA targeting effector protein, it can be guided to the nucleus, allowing nuclear RNAs to be targeted. It is for instance envisaged to probe the function of lincRNAs. Long intergenic non-coding RNAs (lincRNAs) are a vastly underexplored area of research. Most lincRNAs have as of yet unknown functions which could be studies using the RNA targeting effector protein of the invention.

### Identification of RNA Binding Proteins

Identifying proteins bound to specific RNAs can be useful for understanding the roles of many RNAs. For instance, many lincRNAs associate with transcriptional and epigenetic regulators to control transcription. Understanding what proteins bind to a given lincRNA can help elucidate the components in a given regulatory pathway. A RNA targeting effector protein of the invention can be designed to recruit a biotin ligase to a specific transcript in order to label locally bound proteins with biotin. The proteins can then be pulled down and analyzed by mass spectrometry to identify them.

### Assembly of Complexes on RNA and Substrate Shuttling

RNA targeting effector proteins of the invention can further be used to assemble complexes on RNA. This can be achieved by functionalizing the RNA targeting effector protein with multiple related proteins (e.g. components of a particular synthesis pathway). Alternatively, multiple RNA targeting effector proteins can be functionalized with such different related proteins and targeted to the same or adjacent target RNA. Useful application of assembling complexes on RNA are for instance facilitating substrate shuttling between proteins.

### Synthetic Biology

The development of biological systems has a wide utility, including in clinical applications. It is envisaged that the programmable RNA targeting effector proteins of the invention can be used fused to split proteins of toxic domains for targeted cell death, for instance using cancer-linked RNA as target transcript. Further, pathways involving protein-protein interaction can be influenced in synthetic biological systems with e.g. fusion complexes with the appropriate effectors such as kinases or other enzymes.

### Protein Splicing: Inteins

Protein splicing is a post-translational process in which an intervening polypeptide, referred to as an intein, catalyzes its own excision from the polypeptides flacking it, referred to as exteins, as well as subsequent ligation of the exteins. The assembly of two or more RNA targeting effector proteins as described herein on a target transcript could be used to direct the release of a split intein (Topilina and Mills Mob DNA. 2014 Feb. 4; 5(1):5), thereby allowing for direct computation of the existence of a mRNA transcript and subsequent release of a protein product, such as a metabolic enzyme or a transcription factor (for downstream actuation of transcription pathways). This application may have significant relevance in synthetic biology (see above) or large-scale bioproduction (only produce product under certain conditions).

### Inducible, Dosed and Self-Inactivating Systems

In one embodiment, fusion complexes comprising an RNA targeting effector protein of the invention and an effector component are designed to be inducible, for instance light inducible or chemically inducible. Such inducibility allows for activation of the effector component at a desired moment in time.

Light inducibility is for instance achieved by designing a fusion complex wherein CRY2PH1R/CIBN pairing is used for fusion. This system is particularly useful for light induction of protein interactions in living cells (Konermann S, et al. Nature. 2013; 500:472-476).

Chemical inducibility is for instance provided for by designing a fusion complex wherein FKBP/FRB (FK506 binding protein/FKBP rapamycin binding) pairing is used for fusion. Using this system rapamycin is required for binding of proteins (Zetsche et al. Nat Biotechnol. 2015; 33(2):139-42 describes the use of this system for Cas9).

Further, when introduced in the cell as DNA, the RNA targeting effector protein of the inventions can be modulated by inducible promoters, such as tetracycline or doxycycline controlled transcriptional activation (Tet-On and Tet-Off expression system), hormone inducible gene expression system such as for instance an ecdysone inducible gene expression system and an arabinose-inducible gene expression system. When delivered as RNA, expression of the RNA targeting effector protein can be modulated via a riboswitch, which can sense a small molecule like tetracycline (as described in Goldfless et al. Nucleic Acids Res. 2012; 40(9):e64).

In one embodiment, the delivery of the RNA targeting effector protein of the invention can be modulated to change the amount of protein or crRNA in the cell, thereby changing the magnitude of the desired effect or any undesired off-target effects.

In one embodiment, the RNA targeting effector proteins described herein can be designed to be self-inactivating. When delivered to a cell as RNA, either mRNA or as a replication RNA therapeutic (Wrobleska et al NatBiotechnol. 2015 August; 33(8): 839-841), they can self-inactivate expression and subsequent effects by destroying the own RNA, thereby reducing residency and potential undesirable effects.

For further in vivo applications of RNA targeting effector proteins as described herein, reference is made to Mackay J P et al (Nat Struct Mol Biol. 2011 March; 18(3):256-61), Nelles et al (Bioessays. 2015 July; 37(7):732-9) and Abil Z and Zhao H (Mol Biosyst. 2015 October; 11(10):2658-65), which are incorporated herein by reference. In particular, the following applications are envisaged in certain embodiments of the invention, preferably in certain embodiments by using catalytically inactive CRISPR-Cas: enhancing translation (e.g. CRISPR-Cas—translation promotion factor fusions (e.g. eIF4 fusions)); repressing translation (e.g. gRNA targeting ribosome binding sites); exon skipping (e.g. gRNAs targeting splice donor and/or acceptor sites); exon inclusion (e.g. gRNA targeting a particular exon splice donor and/or acceptor site to be included or CRISPR-Cas fused to or recruiting spliceosome components (e.g. U1 snRNA)); accessing RNA localization (e.g. CRISPR-Cas—marker fusions (e.g. EGFP fusions)); altering RNA localization (e.g. CRISPR-Cas—localization signal fusions (e.g. NLS or NES fusions)); RNA degradation (in this case no catalytically inactive CRISPR-Cas is to be used if relied on the activity of CRISPR-Cas, alternatively and for increased specificity, a split CRISPR-Cas may be used); inhibition of non-coding RNA function (e.g. miRNA), such as by degradation or binding of gRNA to functional sites (possibly titrating out at specific sites by relocalization by CRISPR-Cas-signal sequence fusions).

As described herein before and demonstrated in the Examples, CRISPR-Cas function is robust to 5′ or 3′ extensions of the crRNA and to extension of the crRNA loop. It is therefore envisaging that MS2 loops and other recruitment domains can be added to the crRNA without affecting complex formation and binding to target transcripts. Such modifications to the crRNA for recruitment of various effector domains are applicable in the uses of a RNA targeted effector proteins described above.

CRISPR-Cas is capable of mediating resistance to RNA phages. It is therefore envisaged that CRISPR-Cas can be used to immunize, e.g. animals, humans and plants, against RNA-only pathogens, including but not limited to Ebola virus and Zika virus.

In certain embodiments, CRISPR-Cas can process (cleave) its own array. This applies to both the wildtype CRISPR-Cas protein and the mutated CRISPR-Cas protein containing one or more mutated amino acid residues as herein-discussed. It is therefore envisaged that multiple crRNAs designed for different target transcripts and/or applications can be delivered as a single pre-crRNA or as a single transcript driven by one promotor. Such method of delivery has the advantages that it is substantially more compact, easier to synthesize and easier to delivery in viral systems. It will be understood that exact amino acid positions may vary for orthologs of a herein CRISPR-Cas can be adequately determined by protein alignment, as is known in the art, and as described herein elsewhere. Aspects of the invention also encompass methods and uses of the compositions and systems described herein in genome engineering, e.g. for altering or manipulating the expression of one or more genes or the one or more gene products, in prokaryotic or eukaryotic cells, in vitro, in vivo or ex vivo.

In an aspect, the invention provides methods and compositions for modulating, e.g., reducing, expression of a target RNA in cells. In the subject methods, a CRISPR-Cas system of the invention is provided that interferes with transcription, stability, and/or translation of an RNA.

In certain embodiments, an effective amount of CRISPR-Cas system is used to cleave RNA or otherwise inhibit RNA expression. In this regard, the system has uses similar to siRNA and shRNA, thus can also be substituted for such methods. The method includes, without limitation, use of a CRISPR-Cas system as a substitute for e.g., an interfering ribonucleic acid (such as an siRNA or shRNA) or a transcription template thereof, e.g., a DNA encoding an shRNA. The CRISPR-Cas system is introduced into a target cell, e.g., by being administered to a mammal that includes the target cell.

Advantageously, a CRISPR-Cas system of the invention is specific. For example, whereas interfering ribonucleic acid (such as an siRNA or shRNA) polynucleotide systems are plagued by design and stability issues and off-target binding, a CRISPR-Cas system of the invention can be designed with high specificity.

In an aspect of the invention, novel RNA targeting systems also referred to as RNA- or RNA-targeting CRISPR systems of the present application are based on herein-identified CRISPR-Cas proteins which do not require the generation of customized proteins to target specific RNA sequences but rather a single enzyme can be programmed by a RNA molecule to recognize a specific RNA target, in other words the enzyme can be recruited to a specific RNA target using said RNA molecule.

In some embodiments, one or more elements of a nucleic acid-targeting system is derived from a particular organism comprising an endogenous CRISPR RNA-targeting system. In certain embodiments, the CRISPR RNA-targeting system is found in Eubacterium and Ruminococcus. In certain embodiments, the effector protein comprises targeted and collateral ssRNA cleavage activity. In certain embodiments, the effector protein comprises dual HEPN domains. In certain embodiments, the effector protein lacks a counterpart to the Helical-1 domain of Cas13a. In certain embodiments, the effector protein is smaller than previously characterized class 2 CRISPR effectors, with a median size of 928 aa. This median size is 190 aa (17%) less than that of Cas13c, more than 200 aa (18%) less than that of Cas13b, and more than 300 aa (26%) less than that of Cas13a. In certain embodiments, the effector protein has no requirement for a flanking sequence (e.g., PFS, PAM).

In certain embodiments, the effector protein locus structures include a WYL domain containing accessory protein (so denoted after three amino acids that were conserved in the originally identified group of these domains; see, e.g., WYL domain IPR026881). In certain embodiments, the WYL domain accessory protein comprises at least one helix-turn-helix (HTH) or ribbon-helix-helix (RHH) DNA-binding domain. In certain embodiments, the WYL domain containing accessory protein increases both the targeted and the collateral ssRNA cleavage activity of the RNA-targeting effector protein. In certain embodiments, the WYL domain containing accessory protein comprises an N-terminal RHH domain, as well as a pattern of primarily hydrophobic conserved residues, including an invariant tyrosine-leucine doublet corresponding to the original WYL motif. In certain embodiments, the WYL domain containing accessory protein is WYL1. WYL1 is a single WYL-domain protein associated primarily with Ruminococcus.

In other example embodiments, the Type VI RNA-targeting Cas enzyme is Cas 13d. In certain embodiments, Cas13d is Eubacterium siraeum DSM 15702 (EsCas13d) or Ruminococcus sp. N15.MGS-57 (RspCas13d) (see, e.g., Yan et al., Cas13d Is a Compact RNA-Targeting Type VI CRISPR Effector Positively Modulated by a WYL-Domain-Containing Accessory Protein, Molecular Cell (2018), doi.org/10.1016/j.molcel.2018.02.028). RspCas13d and EsCas13d have no flanking sequence requirements (e.g., PFS, PAM).

### Application of the Cas Proteins in Optimized Functional RNA Targeting Systems

In an aspect the invention provides a system for specific delivery of functional components to the RNA environment. This can be ensured using the CRISPR systems comprising the RNA targeting effector proteins of the present invention which allow specific targeting of different components to RNA. More particularly such components include activators or repressors, such as activators or repressors of RNA translation, degradation, etc. Applications of this system are described elsewhere herein.

According to one aspect the invention provides non-naturally occurring or engineered composition comprising a guide RNA comprising a guide sequence capable of hybridizing to a target sequence in a genomic locus of interest in a cell, wherein the guide RNA is modified by the insertion of one or more distinct RNA sequence(s) that bind an adaptor protein. In particular embodiments, the RNA sequences may bind to two or more adaptor proteins (e.g. aptamers), and wherein each adaptor protein is associated with one or more functional domains. The guide RNAs of the CRISPR-Cas enzymes described herein are shown to be amenable to modification of the guide sequence. In particular embodiments, the guide RNA is modified by the insertion of distinct RNA sequence(s) 5′ of the direct repeat, within the direct repeat, or 3′ of the guide sequence. When there is more than one functional domain, the functional domains can be same or different, e.g., two of the same or two different activators or repressors. In an aspect the invention provides a herein-discussed composition, wherein the one or more functional domains are attached to the RNA targeting enzyme so that upon binding to the target RNA the functional domain is in a spatial orientation allowing for the functional domain to function in its attributed function; In an aspect the invention provides a herein-discussed composition, wherein the composition comprises a CRISPR-Cas complex having at least three functional domains, at least one of which is associated with the RNA targeting enzyme and at least two of which are associated with the gRNA.

Accordingly, in an aspect the invention provides non-naturally occurring or engineered CRISPR-Cas complex composition comprising the guide RNA as herein-discussed and a CRISPR-Cas which is an RNA targeting enzyme, wherein optionally the RNA targeting enzyme comprises at least one mutation, such that the RNA targeting enzyme has no more than 5% of the nuclease activity of the enzyme not having the at least one mutation, and optionally one or more comprising at least one or more nuclear localization sequences. In particular embodiments, the guide RNA is additionally or alternatively modified so as to still ensure binding of the RNA targeting enzyme but to prevent cleavage by the RNA targeting enzyme (as detailed elsewhere herein).

In particular embodiments, the RNA targeting enzyme is a CRISPR-Cas protein which has a diminished nuclease activity of at least 97%, or 100% as compared with the CRISPR-Cas enzyme not having the at least one mutation. In an aspect the invention provides a herein-discussed composition, wherein the CRISPR-Cas enzyme comprises two or more mutations as otherwise herein-discussed.

In particular embodiments, an RNA targeting system is provided as described herein above comprising two or more functional domains. In particular embodiments, the two or more functional domains are heterologous functional domain. In particular embodiments, the system comprises an adaptor protein which is a fusion protein comprising a functional domain, the fusion protein optionally comprising a linker between the adaptor protein and the functional domain. In particular embodiments, the linker includes a GlySer linker. Additionally or alternatively, one or more functional domains are attached to the RNA effector protein by way of a linker, optionally a GlySer linker. In particular embodiments, the one or more functional domains are attached to the RNA targeting enzyme through one or both of the HEPN domains.

In an aspect the invention provides a herein-discussed composition, wherein the one or more functional domains associated with the adaptor protein or the RNA targeting enzyme is a domain capable of activating or repressing RNA translation. In an aspect the invention provides a herein-discussed composition, wherein at least one of the one or more functional domains associated with the adaptor protein have one or more activities comprising methylase activity, demethylase activity, transcription activation activity, transcription repression activity, transcription release factor activity, histone modification activity, DNA integration activity RNA cleavage activity, DNA cleavage activity or nucleic acid binding activity, or molecular switch activity or chemical inducibility or light inducibility.

In an aspect the invention provides a herein-discussed composition comprising an aptamer sequence. In particular embodiments, the aptamer sequence is two or more aptamer sequences specific to the same adaptor protein. In an aspect the invention provides a herein-discussed composition, wherein the aptamer sequence is two or more aptamer sequences specific to different adaptor protein. In an aspect the invention provides a herein-discussed composition, wherein the adaptor protein comprises MS2, PP7, Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s, PRR1. Accordingly, in particular embodiments, the aptamer is selected from a binding protein specifically binding any one of the adaptor proteins listed above. In an aspect the invention provides a herein-discussed composition, wherein the cell is a eukaryotic cell. In an aspect the invention provides a herein-discussed composition, wherein the eukaryotic cell is a mammalian cell, a plant cell or a yeast cell, whereby the mammalian cell is optionally a mouse cell. In an aspect the invention provides a herein-discussed composition, wherein the mammalian cell is a human cell.

In an aspect the invention provides a herein above-discussed composition wherein there is more than one guide RNA or gRNA or crRNA, and these target different sequences whereby when the composition is employed, there is multiplexing. In an aspect the invention provides a composition wherein there is more than one guide RNA or gRNA or crRNA modified by the insertion of distinct RNA sequence(s) that bind to one or more adaptor proteins.

In an aspect the invention provides a herein-discussed composition wherein one or more adaptor proteins associated with one or more functional domains is present and bound to the distinct RNA sequence(s) inserted into the guide RNA(s).

In an aspect the invention provides a herein-discussed composition wherein the guide RNA is modified to have at least one non-coding functional loop; e.g., wherein the at least one non-coding functional loop is repressive; for instance, wherein at least one non-coding functional loop comprises Alu.

In an aspect the invention provides a method for modifying gene expression comprising the administration to a host or expression in a host in vivo of one or more of the compositions as herein-discussed.

In an aspect the invention provides a herein-discussed method comprising the delivery of the composition or nucleic acid molecule(s) coding therefor, wherein said nucleic acid molecule(s) are operatively linked to regulatory sequence(s) and expressed in vivo. In an aspect the invention provides a herein-discussed method wherein the expression in vivo is via a lentivirus, an adenovirus, or an AAV.

In an aspect the invention provides a mammalian cell line of cells as herein-discussed, wherein the cell line is, optionally, a human cell line or a mouse cell line. In an aspect the invention provides a transgenic mammalian model, optionally a mouse, wherein the model has been transformed with a herein-discussed composition or is a progeny of said transformant.

In an aspect the invention provides a nucleic acid molecule(s) encoding guide RNA or the RNA targeting CRISPR-Cas complex or the composition as herein-discussed. In an aspect the invention provides a vector comprising: a nucleic acid molecule encoding a guide RNA (gRNA) or crRNA comprising a guide sequence capable of hybridizing to an RNA target sequence in a cell, wherein the direct repeat of the gRNA or crRNA is modified by the insertion of distinct RNA sequence(s) that bind(s) to two or more adaptor proteins, and wherein each adaptor protein is associated with one or more functional domains; or, wherein the gRNA is modified to have at least one non-coding functional loop. In an aspect the invention provides vector(s) comprising nucleic acid molecule(s) encoding: non-naturally occurring or engineered CRISPR-Cas complex composition comprising the gRNA or crRNA herein-discussed, and an RNA targeting enzyme, wherein optionally the RNA targeting enzyme comprises at least one mutation, such that the RNA targeting enzyme has no more than 5% of the nuclease activity of the RNA targeting enzyme not having the at least one mutation, and optionally one or more comprising at least one or more nuclear localization sequences. In an aspect a vector can further comprise regulatory element(s) operable in a eukaryotic cell operably linked to the nucleic acid molecule encoding the guide RNA (gRNA) or crRNA and/or the nucleic acid molecule encoding the RNA targeting enzyme and/or the optional nuclear localization sequence(s).

In one aspect, the invention provides a kit comprising one or more of the components described herein. In some embodiments, the kit comprises a vector system as described herein and instructions for using the kit.

In an aspect the invention provides a method of screening for gain of function (GOF) or loss of function (LOF) or for screening non-coding RNAs or potential regulatory regions (e.g. enhancers, repressors) comprising the cell line of as herein-discussed or cells of the model herein-discussed containing or expressing the RNA targeting enzyme and introducing a composition as herein-discussed into cells of the cell line or model, whereby the gRNA or crRNA includes either an activator or a repressor, and monitoring for GOF or LOF respectively as to those cells as to which the introduced gRNA or crRNA includes an activator or as to those cells as to which the introduced gRNA or crRNA includes a repressor.

In an aspect the invention provides a library of non-naturally occurring or engineered compositions, each comprising a RNA targeting CRISPR guide RNA (gRNA) or crRNA comprising a guide sequence capable of hybridizing to a target RNA sequence of interest in a cell, an RNA targeting enzyme, wherein the RNA targeting enzyme comprises at least one mutation, such that the RNA targeting enzyme has no more than 5% of the nuclease activity of the RNA targeting enzyme not having the at least one mutation, wherein the gRNA or crRNA is modified by the insertion of distinct RNA sequence(s) that bind to one or more adaptor proteins, and wherein the adaptor protein is associated with one or more functional domains, wherein the composition comprises one or more or two or more adaptor proteins, wherein the each protein is associated with one or more functional domains, and wherein the gRNAs or crRNAs comprise a genome wide library comprising a plurality of RNA targeting guide RNAs (gRNAs) or crRNAs. In an aspect the invention provides a library as herein-discussed, wherein the RNA targeting RNA targeting enzyme has a diminished nuclease activity of at least 97%, or 100% as compare with the RNA targeting enzyme not having the at least one mutation. In an aspect the invention provides a library as herein-discussed, wherein the adaptor protein is a fusion protein comprising the functional domain. In an aspect the invention provides a library as herein discussed, wherein the gRNA or crRNA is not modified by the insertion of distinct RNA sequence(s) that bind to the one or two or more adaptor proteins. In an aspect the invention provides a library as herein discussed, wherein the one or two or more functional domains are associated with the RNA targeting enzyme. In an aspect the invention provides a library as herein discussed, wherein the cell population of cells is a population of eukaryotic cells. In an aspect the invention provides a library as herein discussed, wherein the eukaryotic cell is a mammalian cell, a plant cell or a yeast cell. In an aspect the invention provides a library as herein discussed, wherein the mammalian cell is a human cell. In an aspect the invention provides a library as herein discussed, wherein the population of cells is a population of embryonic stem (ES) cells.

In an aspect the invention provides a library as herein discussed, wherein the targeting is of about 100 or more RNA sequences. In an aspect the invention provides a library as herein discussed, wherein the targeting is of about 1000 or more RNA sequences. In an aspect the invention provides a library as herein discussed, wherein the targeting is of about 20,000 or more sequences. In an aspect the invention provides a library as herein discussed, wherein the targeting is of the entire transcriptome. In an aspect the invention provides a library as herein discussed, wherein the targeting is of a panel of target sequences focused on a relevant or desirable pathway. In an aspect the invention provides a library as herein discussed, wherein the pathway is an immune pathway. In an aspect the invention provides a library as herein discussed, wherein the pathway is a cell division pathway.

In one aspect, the invention provides a method of generating a model eukaryotic cell comprising a gene with modified expression. In some embodiments, a disease gene is any gene associated an increase in the risk of having or developing a disease. In some embodiments, the method comprises (a) introducing one or more vectors encoding the components of the system described herein above into a eukaryotic cell, and (b) allowing a CRISPR complex to bind to a target polynucleotide so as to modify expression of a gene, thereby generating a model eukaryotic cell comprising modified gene expression.

The structural information provided herein allows for interrogation of guide RNA or crRNA interaction with the target RNA and the RNA targeting enzyme permitting engineering or alteration of guide RNA structure to optimize functionality of the entire RNA targeting CRISPR-Cas system. For example, the guide RNA or crRNA may be extended, without colliding with the RNA targeting protein by the insertion of adaptor proteins that can bind to RNA. These adaptor proteins can further recruit effector proteins or fusions which comprise one or more functional domains.

An aspect of the invention is that the above elements are comprised in a single composition or comprised in individual compositions. These compositions may advantageously be applied to a host to elicit a functional effect on the genomic level.

The skilled person will understand that modifications to the guide RNA or crRNA which allow for binding of the adapter+functional domain but not proper positioning of the adapter+functional domain (e.g. due to steric hindrance within the three dimension structure of the CRISPR-Cas complex) are modifications which are not intended. The one or more modified guide RNA or crRNA may be modified, by introduction of a distinct RNA sequence(s) 5′ of the direct repeat, within the direct repeat, or 3′ of the guide sequence.

The modified guide RNA or crRNA, the inactivated RNA targeting enzyme (with or without functional domains), and the binding protein with one or more functional domains, may each individually be comprised in a composition and administered to a host individually or collectively. Alternatively, these components may be provided in a single composition for administration to a host. Administration to a host may be performed via viral vectors known to the skilled person or described herein for delivery to a host (e.g. lentiviral vector, adenoviral vector, AAV vector). As explained herein, use of different selection markers (e.g. for lentiviral gRNA or crRNA selection) and concentration of gRNA or crRNA (e.g. dependent on whether multiple gRNAs or crRNAs are used) may be advantageous for eliciting an improved effect.

Using the provided compositions, the person skilled in the art can advantageously and specifically target single or multiple loci with the same or different functional domains to elicit one or more genomic events. The compositions may be applied in a wide variety of methods for screening in libraries in cells and functional modeling in vivo (e.g. gene activation of lincRNA and identification of function; gain-of-function modeling; loss-of-function modeling; the use the compositions of the invention to establish cell lines and transgenic animals for optimization and screening purposes).

The current invention comprehends the use of the compositions of the current invention to establish and utilize conditional or inducible CRISPR-Cas RNA targeting events. (See, e.g., Platt et al., Cell (2014), dx.doi.org/10.1016/j.cell.2014.09.014, or PCT patent publications cited herein, such as WO 2014/093622 (PCT/US2013/074667), which are not believed prior to the present invention or application).

### Applications in Plants and Fungi

The compositions, systems, and methods described herein can be used to perform gene or genome interrogation or editing or manipulation in plants and fungi. For example, the applications include investigation and/or selection and/or interrogations and/or comparison and/or manipulations and/or transformation of plant genes or genomes; e.g., to create, identify, develop, optimize, or confer trait(s) or characteristic(s) to plant(s) or to transform a plant or fungus genome. There can accordingly be improved production of plants, new plants with new combinations of traits or characteristics or new plants with enhanced traits. The compositions, systems, and methods can be used with regard to plants in Site-Directed Integration (SDI) or Gene Editing (GE) or any Near Reverse Breeding (NRB) or Reverse Breeding (RB) techniques.

The compositions, systems, and methods herein may be used to confer desired traits (e.g., enhanced nutritional quality, increased resistance to diseases and resistance to biotic and abiotic stress, and increased production of commercially valuable plant products or heterologous compounds) on essentially any plants and fungi, and their cells and tissues. The compositions, systems, and methods may be used to modify endogenous genes or to modify their expression without the permanent introduction into the genome of any foreign gene.

In some embodiments, compositions, systems, and methods may be used in genome editing in plants or where RNAi or similar genome editing techniques have been used previously; see, e.g., Nekrasov, “Plant genome editing made easy: targeted mutagenesis in model and crop plants using the CRISPR-Cas system,” Plant Methods 2013, 9:39 (doi:10.1186/1746-4811-9-39); Brooks, “Efficient gene editing in tomato in the first generation using the CRISPR-Cas9 system,” Plant Physiology September 2014 pp 114.247577; Shan, “Targeted genome modification of crop plants using a CRISPR-Cas system,” Nature Biotechnology 31, 686-688 (2013); Feng, “Efficient genome editing in plants using a CRISPR/Cas system,” Cell Research (2013) 23:1229-1232. doi:10.1038/cr.2013.114; published online 20 Aug. 2013; Xie, “RNA-guided genome editing in plants using a CRISPR-Cas system,” Mol Plant. 2013 November; 6(6):1975-83. doi: 10.1093/mp/sst119. Epub 2013 Aug. 17; Xu, “Gene targeting using the Agrobacterium tumefaciens-mediated CRISPR-Cas system in rice,” Rice 2014, 7:5 (2014), Zhou et al., “Exploiting SNPs for biallelic CRISPR mutations in the outcrossing woody perennial Populus reveals 4-coumarate: CoA ligase specificity and Redundancy,” New Phytologist (2015) (Forum) 1-4 (available online only at www.newphytologist.com); Caliando et al, “Targeted DNA degradation using a CRISPR device stably carried in the host genome, NATURE COMMUNICATIONS 6:6989, DOI: 10.1038/ncomms7989, www.nature.com/naturecommunications DOI: 10.1038/ncomms7989; U.S. Pat. No. 6,603,061—Agrobacterium-Mediated Plant Transformation Method; U.S. Pat. No. 7,868,149—Plant Genome Sequences and Uses Thereof and US 2009/0100536-Transgenic Plants with Enhanced Agronomic Traits, Morrell et al “Crop genomics: advances and applications,” Nat Rev Genet. 2011 Dec. 29; 13(2):85-96, all the contents and disclosure of each of which are herein incorporated by reference in their entirety. Aspects of utilizing the compositions, systems, and methods may be analogous to the use of the CRISPR-Cas system in plants, and mention is made of the University of Arizona website “CRISPR-PLANT” (www.genome.arizona.edu/crispr/) (supported by Penn State and AGI).

The compositions, systems, and methods may also be used on protoplasts. A “protoplast” refers to a plant cell that has had its protective cell wall completely or partially removed using, for example, mechanical or enzymatic means resulting in an intact biochemical competent unit of living plant that can reform their cell wall, proliferate and regenerate grow into a whole plant under proper growing conditions.

The compositions, systems, and methods may be used for screening genes (e.g., endogenous, mutations) of interest. In some examples, genes of interest include those encoding enzymes involved in the production of a component of added nutritional value or generally genes affecting agronomic traits of interest, across species, phyla, and plant kingdom. By selectively targeting e.g. genes encoding enzymes of metabolic pathways, the genes responsible for certain nutritional aspects of a plant can be identified. Similarly, by selectively targeting genes which may affect a desirable agronomic trait, the relevant genes can be identified. Accordingly, the present invention encompasses screening methods for genes encoding enzymes involved in the production of compounds with a particular nutritional value and/or agronomic traits.

It is also understood that reference herein to animal cells may also apply, mutatis mutandis, to plant or fungal cells unless otherwise apparent; and, the enzymes herein having reduced off-target effects and systems employing such enzymes can be used in plant applications, including those mentioned herein.

In some cases, nucleic acids introduced to plants and fungi may be codon optimized for expression in the plants and fungi. Methods of codon optimization include those described in Kwon K. C., et al., Codon Optimization to Enhance Expression Yields Insights into Chloroplast Translation, Plant Physiol. 2016 September; 172(1):62-77.

The components (e.g., Cas proteins) in the compositions and systems may further comprise one or more functional domains described herein. In some examples, the functional domains may be an exonuclease. Such exonuclease may increase the efficiency of the Cas proteins' function, e.g., mutagenesis efficiency. An example of the functional domain is Trex2, as described in Weiss T et al., www.biorxiv.org/content/10.1101/2020.04.11.037572v1, doi: doi.org/10.1101/2020.04.11.037572.

### Examples of Plants

The compositions, systems, and methods herein can be used to confer desired traits on essentially any plant. A wide variety of plants and plant cell systems may be engineered for the desired physiological and agronomic characteristics. In general, the term “plant” relates to any various photosynthetic, eukaryotic, unicellular or multicellular organism of the kingdom Plantae characteristically growing by cell division, containing chloroplasts, and having cell walls comprised of cellulose. The term plant encompasses monocotyledonous and dicotyledonous plants.

The compositions, systems, and methods may be used over a broad range of plants, such as for example with dicotyledonous plants belonging to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales; monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g., those belonging to the orders Pinales, Ginkgoales, Cycadales, Araucariales, Cupressales and Gnetales.

The compositions, systems, and methods herein can be used over a broad range of plant species, included in the non-limitative list of dicot, monocot or gymnosperm genera hereunder: Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, and Pseudotsuga.

In some embodiments, target plants and plant cells for engineering include those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Specifically, the plants are intended to comprise without limitation angiosperm and gymnosperm plants such as acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini.

The term plant also encompasses Algae, which are mainly photoautotrophs unified primarily by their lack of roots, leaves and other organs that characterize higher plants. The compositions, systems, and methods can be used over a broad range of “algae” or “algae cells.” Examples of algae include eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). Examples of algae species include those of Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

### Plant Promoters

In order to ensure appropriate expression in a plant cell, the components of the components and systems herein may be placed under control of a plant promoter. A plant promoter is a promoter operable in plant cells. A plant promoter is capable of initiating transcription in plant cells, whether or not its origin is a plant cell. The use of different types of promoters is envisaged.

In some examples, the plant promoter is a constitutive plant promoter, which is a promoter that is able to express the open reading frame (ORF) that it controls in all or nearly all of the plant tissues during all or nearly all developmental stages of the plant (referred to as “constitutive expression”). One example of a constitutive promoter is the cauliflower mosaic virus 35S promoter. In some examples, the plant promoter is a regulated promoter, which directs gene expression not constitutively, but in a temporally- and/or spatially-regulated manner, and includes tissue-specific, tissue-preferred and inducible promoters. Different promoters may direct the expression of a gene in different tissues or cell types, or at different stages of development, or in response to different environmental conditions. In some examples, the plant promoter is a tissue-preferred promoter, which can be utilized to target enhanced expression in certain cell types within a particular plant tissue, for instance vascular cells in leaves or roots or in specific cells of the seed.

Exemplary plant promoters include those obtained from plants, plant viruses, and bacteria such as Agrobacterium or Rhizobium which comprise genes expressed in plant cells. Additional examples of promoters include those described in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

In some examples, a plant promoter may be an inducible promoter, which is inducible and allows for spatiotemporal control of gene editing or gene expression may use a form of energy. The form of energy may include sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. In a particular example, of the components of a light inducible system include a Cas protein, a light-responsive cytochrome heterodimer (e.g. from Arabidopsis thaliana), and a transcriptional activation/repression domain.

In some examples, the promoter may be a chemical-regulated promotor (where the application of an exogenous chemical induces gene expression) or a chemical-repressible promoter (where application of the chemical represses gene expression). Examples of chemical-inducible promoters include maize ln2-2 promoter (activated by benzene sulfonamide herbicide safeners), the maize GST promoter (activated by hydrophobic electrophilic compounds used as pre-emergent herbicides), the tobacco PR-1 a promoter (activated by salicylic acid), promoters regulated by antibiotics (such as tetracycline-inducible and tetracycline-repressible promoters).

### Stable Integration in the Genome of Plants

In some embodiments, polynucleotides encoding the components of the compositions and systems may be introduced for stable integration into the genome of a plant cell. In some cases, vectors or expression systems may be used for such integration. The design of the vector or the expression system can be adjusted depending on for when, where and under what conditions the guide RNA and/or the Cas gene are expressed. In some cases, the polynucleotides may be integrated into an organelle of a plant, such as a plastid, mitochondrion or a chloroplast. The elements of the expression system may be on one or more expression constructs which are either circular such as a plasmid or transformation vector, or non-circular such as linear double stranded DNA.

In some embodiments, the method of integration generally comprises the steps of selecting a suitable host cell or host tissue, introducing the construct(s) into the host cell or host tissue, and regenerating plant cells or plants therefrom. In some examples, the expression system for stable integration into the genome of a plant cell may contain one or more of the following elements: a promoter element that can be used to express the RNA and/or Cas enzyme in a plant cell; a 5′ untranslated region to enhance expression; an intron element to further enhance expression in certain cells, such as monocot cells; a multiple-cloning site to provide convenient restriction sites for inserting the guide RNA and/or the Cas gene sequences and other desired elements; and a 3′ untranslated region to provide for efficient termination of the expressed transcript.

### Transient Expression in Plants

In some embodiments, the components of the compositions and systems may be transiently expressed in the plant cell. In some examples, the compositions and systems may modify a target nucleic acid only when both the guide RNA and the Cas protein are present in a cell, such that genomic modification can further be controlled. As the expression of the Cas protein is transient, plants regenerated from such plant cells typically contain no foreign DNA. In certain examples, the Cas protein is stably expressed and the guide sequence is transiently expressed.

DNA and/or RNA (e.g., mRNA) may be introduced to plant cells for transient expression. In such cases, the introduced nucleic acid may be provided in sufficient quantity to modify the cell but do not persist after a contemplated period of time has passed or after one or more cell divisions.

The transient expression may be achieved using suitable vectors. Exemplary vectors that may be used for transient expression include a pEAQ vector (may be tailored for Agrobacterium-mediated transient expression) and Cabbage Leaf Curl virus (CaLCuV), and vectors described in Sainsbury F. et al., Plant Biotechnol J. 2009 September; 7(7):682-93; and Yin K et al., Scientific Reports volume 5, Article number: 14926 (2015).

Combinations of the different methods described above are also envisaged.

Translocation to and/or Expression in Specific Plant Organelles

The compositions and systems herein may comprise elements for translocation to and/or expression in a specific plant organelle.

### Chloroplast Targeting

In some embodiments, it is envisaged that the compositions and systems are used to specifically modify chloroplast genes or to ensure expression in the chloroplast. The compositions and systems (e.g., Cas proteins, guide molecules, or their encoding polynucleotides) may be transformed, compartmentalized, and/or targeted to the chloroplast. In an example, the introduction of genetic modifications in the plastid genome can reduce biosafety issues such as gene flow through pollen.

Examples of methods of chloroplast transformation include Particle bombardment, PEG treatment, and microinjection, and the translocation of transformation cassettes from the nuclear genome to the plastid. In some examples, targeting of chloroplasts may be achieved by incorporating in chloroplast localization sequence, and/or the expression construct a sequence encoding a chloroplast transit peptide (CTP) or plastid transit peptide, operably linked to the 5′ region of the sequence encoding the components of the compositions and systems. Additional examples of transforming, targeting and localization of chloroplasts include those described in WO2010061186, Protein Transport into Chloroplasts, 2010, Annual Review of Plant Biology, Vol. 61: 157-180, and US 20040142476, which are incorporated by reference herein in their entireties.

### Exemplary Applications in Plants

The compositions, systems, and methods may be used to generate genetic variation(s) in a plant (e.g., crop) of interest. One or more, e.g., a library of, guide molecules targeting one or more locations in a genome may be provided and introduced into plant cells together with the Cas effector protein. For example, a collection of genome-scale point mutations and gene knock-outs can be generated. In some examples, the compositions, systems, and methods may be used to generate a plant part or plant from the cells so obtained and screening the cells for a trait of interest. The target genes may include both coding and non-coding regions. In some cases, the trait is stress tolerance and the method is a method for the generation of stress-tolerant crop varieties.

In some embodiments, the compositions, systems, and methods are used to modify endogenous genes or to modify their expression. The expression of the components may induce targeted modification of the genome, either by direct activity of the Cas nuclease and optionally introduction of recombination template DNA, or by modification of genes targeted. The different strategies described herein above allow Cas-mediated targeted genome editing without requiring the introduction of the components into the plant genome.

In some cases, the modification may be performed without the permanent introduction into the genome of the plant of any foreign gene, including those encoding CRISPR components, so as to avoid the presence of foreign DNA in the genome of the plant. This can be of interest as the regulatory requirements for non-transgenic plants are less rigorous. Components which are transiently introduced into the plant cell are typically removed upon crossing.

For example, the modification may be performed by transient expression of the components of the compositions and systems. The transient expression may be performed by delivering the components of the compositions and systems with viral vectors, delivery into protoplasts, with the aid of particulate molecules such as nanoparticles or CPPs.

Generation of Plants with Desired Traits

The compositions, systems, and methods herein may be used to introduce desired traits to plants. The approaches include introduction of one or more foreign genes to confer a trait of interest, editing or modulating endogenous genes to confer a trait of interest.

### Agronomic Traits

In some embodiments, crop plants can be improved by influencing specific plant traits. Examples of the traits include improved agronomic traits such as herbicide resistance, disease resistance, abiotic stress tolerance, high yield, and superior quality, pesticide-resistance, disease resistance, insect and nematode resistance, resistance against parasitic weeds, drought tolerance, nutritional value, stress tolerance, self-pollination voidance, forage digestibility biomass, and grain yield.

In some embodiments, genes that confer resistance to pests or diseases may be introduced to plants. In cases there are endogenous genes that confer such resistance in a plants, their expression and function may be enhanced (e.g., by introducing extra copies, modifications that enhance expression and/or activity).

Examples of genes that confer resistance include plant disease resistance genes (e.g., Cf-9, Pto, RSP2, S1DMR6-1), genes conferring resistance to a pest (e.g., those described in WO96/30517), Bacillus thuringiensis proteins, lectins, Vitamin-binding proteins (e.g., avidin), enzyme inhibitors (e.g., protease or proteinase inhibitors or amylase inhibitors), insect-specific hormones or pheromones (e.g., ecdysteroid or a juvenile hormone, variant thereof, a mimetic based thereon, or an antagonist or agonist thereof) or genes involved in the production and regulation of such hormone and pheromones, insect-specific peptides or neuropeptide, Insect-specific venom (e.g., produced by a snake, a wasp, etc., or analog thereof), Enzymes responsible for a hyperaccumulation of a monoterpene, a sesquiterpene, a steroid, hydroxamic acid, a phenylpropanoid derivative or another nonprotein molecule with insecticidal activity, Enzymes involved in the modification of biologically active molecule (e.g., a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic), molecules that stimulates signal transduction, Viral-invasive proteins or a complex toxin derived therefrom, Developmental-arrestive proteins produced in nature by a pathogen or a parasite, a developmental-arrestive protein produced in nature by a plant, or any combination thereof.

The compositions, systems, and methods may be used to identify, screen, introduce or remove mutations or sequences lead to genetic variability that give rise to susceptibility to certain pathogens, e.g., host specific pathogens. Such approach may generate plants that are non-host resistance, e.g., the host and pathogen are incompatible or there can be partial resistance against all races of a pathogen, typically controlled by many genes and/or also complete resistance to some races of a pathogen but not to other races.

In some embodiments, the compositions, systems, and methods may be used to modify genes involved in plant diseases. Such genes may be removed, inactivated, or otherwise regulated or modified. Examples of plant diseases include those described in [0045]-[0080] of US20140213619A1, which is incorporated by reference herein in its entirety.

In some embodiments, genes that confer resistance to herbicides may be introduced to plants. Examples of genes that confer resistance to herbicides include genes conferring resistance to herbicides that inhibit the growing point or meristem, such as an imidazolinone or a sulfonylurea, genes conferring glyphosate tolerance (e.g., resistance conferred by, e.g., mutant 5-enolpyruvylshikimate-3-phosphate synthase genes, aroA genes and glyphosate acetyl transferase (GAT) genes, respectively), or resistance to other phosphono compounds such as by glufosinate (phosphinothricin acetyl transferase (PAT) genes from Streptomyces species, including Streptomyces hygroscopicus and Streptomyces viridichromogenes), and to pyridinoxy or phenoxy proprionic acids and cyclohexones by ACCase inhibitor-encoding genes), genes conferring resistance to herbicides that inhibit photosynthesis (such as a triazine (psbA and gs+ genes) or a benzonitrile (nitrilase gene), and glutathione S-transferase), genes encoding enzymes detoxifying the herbicide or a mutant glutamine synthase enzyme that is resistant to inhibition, genes encoding a detoxifying enzyme is an enzyme encoding a phosphinothricin acetyltransferase (such as the bar or pat protein from Streptomyces species), genes encoding hydroxyphenylpyruvatedioxygenases (HPPD) inhibitors, e.g., naturally occurring HPPD resistant enzymes, and genes encoding a mutated or chimeric HPPD enzyme.

In some embodiments, genes involved in Abiotic stress tolerance may be introduced to plants. Examples of genes include those capable of reducing the expression and/or the activity of poly(ADP-ribose) polymerase (PARP) gene, transgenes capable of reducing the expression and/or the activity of the PARG encoding genes, genes coding for a plant-functional enzyme of the nicotineamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphorybosyltransferase, enzymes involved in carbohydrate biosynthesis, enzymes involved in the production of polyfructose (e.g., the inulin and levan-type), the production of alpha-1,6 branched alpha-1,4-glucans, the production of alternan, the production of hyaluronan.

In some embodiments, genes that improve drought resistance may be introduced to plants. Examples of genes Ubiquitin Protein Ligase protein (UPL) protein (UPL3), DR02, DR03, ABC transporter, and DREB1A.

### Nutritionally Improved Plants

In some embodiments, the compositions, systems, and methods may be used to produce nutritionally improved plants. In some examples, such plants may provide functional foods, e.g., a modified food or food ingredient that may provide a health benefit beyond the traditional nutrients it contains. In certain examples, such plants may provide nutraceuticals foods, e.g., substances that may be considered a food or part of a food and provides health benefits, including the prevention and treatment of disease. The nutraceutical foods may be useful in the prevention and/or treatment of diseases in animals and humans, e.g., cancers, diabetes, cardiovascular disease, and hypertension.

An improved plant may naturally produce one or more desired compounds and the modification may enhance the level or activity or quality of the compounds. In some cases, the improved plant may not naturally produce the compound(s), while the modification enables the plant to produce such compound(s). In some cases, the compositions, systems, and methods used to modify the endogenous synthesis of these compounds indirectly, e.g. by modifying one or more transcription factors that controls the metabolism of this compound.

Examples of nutritionally improved plants include plants comprising modified protein quality, content and/or amino acid composition, essential amino acid contents, oils and fatty acids, carbohydrates, vitamins and carotenoids, functional secondary metabolites, and minerals. In some examples, the improved plants may comprise or produce compounds with health benefits. Examples of nutritionally improved plants include those described in Newell-McGloughlin, Plant Physiology, July 2008, Vol. 147, pp. 939-953.

Examples of compounds that can be produced include carotenoids (e.g., α-Carotene or β-Carotene), lutein, lycopene, Zeaxanthin, Dietary fiber (e.g., insoluble fibers, β-Glucan, soluble fibers, fatty acids (e.g., ω-3 fatty acids, Conjugated linoleic acid, GLA,), Flavonoids (e.g., Hydroxycinnamates, flavonols, catechins and tannins), Glucosinolates, indoles, isothiocyanates (e.g., Sulforaphane), Phenolics (e.g., stilbenes, caffeic acid and ferulic acid, epicatechin), Plant stanols/sterols, Fructans, inulins, fructo-oligosaccharides, Saponins, Soybean proteins, Phytoestrogens (e.g., isoflavones, lignans), Sulfides and thiols such as diallyl sulphide, Allyl methyl trisulfide, dithiolthiones, Tannins, such as proanthocyanidins, or any combination thereof.

The compositions, systems, and methods may also be used to modify protein/starch functionality, shelf life, taste/aesthetics, fiber quality, and allergen, antinutrient, and toxin reduction traits.

Examples of genes and nucleic acids that can be modified to introduce the traits include stearyl-ACP desaturase, DNA associated with the single allele which may be responsible for maize mutants characterized by low levels of phytic acid, Tf RAP2.2 and its interacting partner SINAT2, Tf Dof1, and DOF Tf AtDof1.1 (OBP2).

### Modification of Polyploid Plants

The compositions, systems, and methods may be used to modify polyploid plants. Polyploid plants carry duplicate copies of their genomes (e.g. as many as six, such as in wheat). In some cases, the compositions, systems, and methods may be can be multiplexed to affect all copies of a gene, or to target dozens of genes at once. For instance, the compositions, systems, and methods may be used to simultaneously ensure a loss of function mutation in different genes responsible for suppressing defenses against a disease. The modification may be simultaneous suppression the expression of the TaMLO-A1, TaMLO-B1 and TaMLO-D1 nucleic acid sequence in a wheat plant cell and regenerating a wheat plant therefrom, in order to ensure that the wheat plant is resistant to powdery mildew (e.g., as described in WO2015109752).

### Regulation of Fruit-Ripening

The compositions, systems, and methods may be used to regulate ripening of fruits. Ripening is a normal phase in the maturation process of fruits and vegetables. Only a few days after it starts it may render a fruit or vegetable inedible, which can bring significant losses to both farmers and consumers.

In some embodiments, the compositions, systems, and methods are used to reduce ethylene production. In some examples, the compositions, systems, and methods may be used to suppress the expression and/or activity of ACC synthase, insert a ACC deaminase gene or a functional fragment thereof, insert a SAM hydrolase gene or functional fragment thereof, suppress ACC oxidase gene expression

Alternatively or additionally, the compositions, systems, and methods may be used to modify ethylene receptors (e.g., suppressing ETR1) and/or Polygalacturonase (PG). Suppression of a gene may be achieved by introducing a mutation, an antisense sequence, and/or a truncated copy of the gene to the genome.

### Increasing Storage Life of Plants

In some embodiments, the compositions, systems, and methods are used to modify genes involved in the production of compounds which affect storage life of the plant or plant part. The modification may be in a gene that prevents the accumulation of reducing sugars in potato tubers. Upon high-temperature processing, these reducing sugars react with free amino acids, resulting in brown, bitter-tasting products and elevated levels of acrylamide, which is a potential carcinogen. In particular embodiments, the methods provided herein are used to reduce or inhibit expression of the vacuolar invertase gene (VInv), which encodes a protein that breaks down sucrose to glucose and fructose.

### Reducing Allergens in Plants

In some embodiments, the compositions, systems, and methods are used to generate plants with a reduced level of allergens, making them safer for consumers. To this end, the compositions, systems, and methods may be used to identify and modify (e.g., suppress) one or more genes responsible for the production of plant allergens. Examples of such genes include Lol p5, as well as those in peanuts, soybeans, lentils, peas, lupin, green beans, mung beans, such as those described in Nicolaou et al., Current Opinion in Allergy and Clinical Immunology 2011; 11(3):222), which is incorporated by reference herein in its entirety.

### Generation of Male Sterile Plants

The compositions, systems, and methods may be used to generate male sterile plants. Hybrid plants typically have advantageous agronomic traits compared to inbred plants. However, for self-pollinating plants, the generation of hybrids can be challenging. In different plant types (e.g., maize and rice), genes have been identified which are important for plant fertility, more particularly male fertility. Plants that are as such genetically altered can be used in hybrid breeding programs.

The compositions, systems, and methods may be used to modify genes involved male fertility, e.g., inactivating (such as by introducing mutations to) genes required for male fertility. Examples of the genes involved in male fertility include cytochrome P450-like gene (MS26) or the meganuclease gene (MS45), and those described in Wan X et al., Mol Plant. 2019 Mar. 4; 12(3):321-342; and Kim Y. J., et al., Trends Plant Sci. 2018 January; 23(1):53-65.

### Increasing the Fertility Stage in Plants

In some embodiments, the compositions, systems, and methods may be used to prolong the fertility stage of a plant such as of a rice. For instance, a rice fertility stage gene such as Ehd3 can be targeted in order to generate a mutation in the gene and plantlets can be selected for a prolonged regeneration plant fertility stage.

### Production of Early Yield of Products

In some embodiments, the compositions, systems, and methods may be used to produce early yield of the product. For example, flowering process may be modulated, e.g., by mutating flowering repressor gene such as SP5G. Examples of such approaches include those described in Soyk S, et al., Nat Genet. 2017 January; 49(1):162-168.

### Oil and Biofuel Production

The compositions, systems, and methods may be used to generate plants for oil and biofuel production. Biofuels include fuels made from plant and plant-derived resources. Biofuels may be extracted from organic matter whose energy has been obtained through a process of carbon fixation or are made through the use or conversion of biomass. This biomass can be used directly for biofuels or can be converted to convenient energy containing substances by thermal conversion, chemical conversion, and biochemical conversion. This biomass conversion can result in fuel in solid, liquid, or gas form. Biofuels include bioethanol and biodiesel. Bioethanol can be produced by the sugar fermentation process of cellulose (starch), which may be derived from maize and sugar cane. Biodiesel can be produced from oil crops such as rapeseed, palm, and soybean. Biofuels can be used for transportation.

### Generation of Plants for Production of Vegetable Oils and Biofuels

The compositions, systems, and methods may be used to generate algae (e.g., diatom) and other plants (e.g., grapes) that express or overexpress high levels of oil or biofuels.

In some cases, the compositions, systems, and methods may be used to modify genes involved in the modification of the quantity of lipids and/or the quality of the lipids. Examples of such genes include those involved in the pathways of fatty acid synthesis, e.g., acetyl-CoA carboxylase, fatty acid synthase, 3-ketoacyl_acyl-carrier protein synthase III, glycerol-3-phospate deshydrogenase (G3PDH), Enoyl-acyl carrier protein reductase (Enoyl-ACP-reductase), glycerol-3-phosphate acyltransferase, lysophosphatidic acyl transferase or diacylglycerol acyltransferase, phospholipid:diacylglycerol acyltransferase, phoshatidate phosphatase, fatty acid thioesterase such as palmitoyi protein thioesterase, or malic enzyme activities.

In further embodiments, it is envisaged to generate diatoms that have increased lipid accumulation. This can be achieved by targeting genes that decrease lipid catabolization. Examples of genes include those involved in the activation of triacylglycerol and free fatty acids, β-oxidation of fatty acids, such as genes of acyl-CoA synthetase, 3-ketoacyl-CoA thiolase, acyl-CoA oxidase activity and phosphoglucomutase.

In some examples, algae may be modified for production of oil and biofuels, including fatty acids (e.g., fatty esters such as acid methyl esters (FAME) and fatty acid ethyl esters (FAEE)). Examples of methods of modifying microalgae include those described in Stovicek et al. Metab. Eng. Comm., 2015; 2:1; U.S. Pat. No. 8,945,839; and International Patent Publication No. WO 2015/086795.

In some examples, one or more genes may be introduced (e.g., overexpressed) to the plants (e.g., algae) to produce oils and biofuels (e.g., fatty acids) from a carbon source (e.g., alcohol). Examples of the genes include genes encoding acyl-CoA synthases, ester synthases, thioesterases (e.g., tesA, ′tesA, tesB, fatB, fatB2, fatB3, fatA1, or fatA), acyl-CoA synthases (e.g., fadD, JadK, BH3103, pfl-4354, EAV15023, fadD1, fadD2, RPC_4074, fadDD35, fadDD22, faa39), ester synthases (e.g., synthase/acyl-CoA:diacylglycerl acyltransferase from Simmondsia chinensis, Acinetobacter sp. ADP, Alcanivorax borkumensis, Pseudomonas aeruginosa, Fundibacter jadensis, Arabidopsis thaliana, or Alkaligenes eutrophus, or variants thereof).

Additionally or alternatively, one or more genes in the plants (e.g., algae) may be inactivated (e.g., expression of the genes is decreased). For examples, one or more mutations may be introduced to the genes. Examples of such genes include genes encoding acyl-CoA dehydrogenases (e.g., fade), outer membrane protein receptors, and transcriptional regulator (e.g., repressor) of fatty acid biosynthesis (e.g., fabR), pyruvate formate lyases (e.g., pflB), lactate dehydrogenases (e.g., IdhA).

### Organic Acid Production

In some embodiments, plants may be modified to produce organic acids such as lactic acid. The plants may produce organic acids using sugars, pentose or hexose sugars. To this end, one or more genes may be introduced (e.g., and overexpressed) in the plants. An example of such genes include LDH gene.

In some examples, one or more genes may be inactivated (e.g., expression of the genes is decreased). For examples, one or more mutations may be introduced to the genes. The genes may include those encoding proteins involved an endogenous metabolic pathway which produces a metabolite other than the organic acid of interest and/or wherein the endogenous metabolic pathway consumes the organic acid.

Examples of genes that can be modified or introduced include those encoding pyruvate decarboxylases (pdc), fumarate reductases, alcohol dehydrogenases (adh), acetaldehyde dehydrogenases, phosphoenolpyruvate carboxylases (ppc), D-lactate dehydrogenases (d-ldh), L-lactate dehydrogenases (1-ldh), lactate 2-monooxygenases, lactate dehydrogenase, cytochrome-dependent lactate dehydrogenases (e.g., cytochrome B2-dependent L-lactate dehydrogenases).

### Enhancing Plant Properties for Biofuel Production

In some embodiments, the compositions, systems, and methods are used to alter the properties of the cell wall of plants to facilitate access by key hydrolyzing agents for a more efficient release of sugars for fermentation. By reducing the proportion of lignin in a plant the proportion of cellulose can be increased. In particular embodiments, lignin biosynthesis may be downregulated in the plant so as to increase fermentable carbohydrates.

In some examples, one or more lignin biosynthesis genes may be down regulated. Examples of such genes include 4-coumarate 3-hydroxylases (C3H), phenylalanine ammonia-lyases (PAL), cinnamate 4-hydroxylases (C4H), hydroxycinnamoyl transferases (HCT), caffeic acid O-methyltransferases (COMT), caffeoyl CoA 3-O-methyltransferases (CCoAOMT), ferulate 5-hydroxylases (F5H), cinnamyl alcohol dehydrogenases (CAD), cinnamoyl CoA-reductases (CCR), 4-coumarate-CoA ligases (4CL), monolignol-lignin-specific glycosyltransferases, and aldehyde dehydrogenases (ALDH), and those described in WO 2008064289.

In some examples, plant mass that produces lower level of acetic acid during fermentation may be reduced. To this end, genes involved in polysaccharide acetylation (e.g., Cas1L and those described in WO 2010096488) may be inactivated.

### Other Microorganisms for Oils and Biofuel Production

In some embodiments, microorganisms other than plants may be used for production of oils and biofuels using the compositions, systems, and methods herein. Examples of the microorganisms include those of the genus of Escherichia, Bacillus, Lactobacillus, Rhodococcus, Synechococcus, Synechoystis, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces.

### Plant Cultures and Regeneration

In some embodiments, the modified plants or plant cells may be cultured to regenerate a whole plant which possesses the transformed or modified genotype and thus the desired phenotype. Examples of regeneration techniques include those relying on manipulation of certain phytohormones in a tissue culture growth medium, relying on a biocide and/or herbicide marker which has been introduced together with the desired nucleotide sequences, obtaining from cultured protoplasts, plant callus, explants, organs, pollens, embryos or parts thereof.

### Detecting Modifications in the Plant Genome-Selectable Markers

When the compositions, systems, and methods are used to modify a plant, suitable methods may be used to confirm and detect the modification made in the plant. In some examples, when a variety of modifications are made, one or more desired modifications or traits resulting from the modifications may be selected and detected. The detection and confirmation may be performed by biochemical and molecular biology techniques such as Southern analysis, PCR, Northern blot, S1 RNase protection, primer-extension or reverse transcriptase-PCR, enzymatic assays, ribozyme activity, gel electrophoresis, Western blot, immunoprecipitation, enzyme-linked immunoassays, in situ hybridization, enzyme staining, and immunostaining.

In some cases, one or more markers, such as selectable and detectable markers, may be introduced to the plants. Such markers may be used for selecting, monitoring, isolating cells and plants with desired modifications and traits. A selectable marker can confer positive or negative selection and is conditional or non-conditional on the presence of external substrates. Examples of such markers include genes and proteins that confer resistance to antibiotics, such as hygromycin (hpt) and kanamycin (nptII), and genes that confer resistance to herbicides, such as phosphinothricin (bar) and chlorosulfuron (als), enzyme capable of producing or processing a colored substances (e.g., the β-glucuronidase, luciferase, B or C1 genes).

### Applications in Fungi

The compositions, systems, and methods described herein can be used to perform efficient and cost effective gene or genome interrogation or editing or manipulation in fungi or fungal cells, such as yeast. The approaches and applications in plants may be applied to fungi as well.

A fungal cell may be any type of eukaryotic cell within the kingdom of fungi, such as phyla of Ascomycota, Basidiomycota, Blastocladiomycota, Chytridiomycota, Glomeromycota, Microsporidia, and Neocallimastigomycota. Examples of fungi or fungal cells in include yeasts, molds, and filamentous fungi.

In some embodiments, the fungal cell is a yeast cell. A yeast cell refers to any fungal cell within the phyla Ascomycota and Basidiomycota. Examples of yeasts include budding yeast, fission yeast, and mold, S. cerervisiae, Kluyveromyces marxianus, Issatchenkia orientalis, Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, Pichia kudriavzevii and Candida acidothermophilum).

In some embodiments, the fungal cell is a filamentous fungal cell, which grow in filaments, e.g., hyphae or mycelia. Examples of filamentous fungal cells include Aspergillus spp. (e.g., Aspergillus niger), Trichoderma spp. (e.g., Trichoderma reesei), Rhizopus spp. (e.g., Rhizopus or yzae), and Mortierella spp. (e.g., Mortierella isabellina).

In some embodiments, the fungal cell is of an industrial strain. Industrial strains include any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes include fermentation (e.g., in production of food or beverage products), distillation, biofuel production, production of a compound, and production of a polypeptide. Examples of industrial strains include, without limitation, JAY270 and ATCC4124.

In some embodiments, the fungal cell is a polyploid cell whose genome is present in more than one copy. Polyploid cells include cells naturally found in a polyploid state, and cells that has been induced to exist in a polyploid state (e.g., through specific regulation, alteration, inactivation, activation, or modification of meiosis, cytokinesis, or DNA replication). A polyploid cell may be a cell whose entire genome is polyploid, or a cell that is polyploid in a particular genomic locus of interest. In some examples, the abundance of guide RNA may more of ten be a rate-limiting component in genome engineering of polyploid cells than in haploid cells, and thus the methods using the CRISPR system described herein may take advantage of using certain fungal cell types.

In some embodiments, the fungal cell is a diploid cell, whose genome is present in two copies. Diploid cells include cells naturally found in a diploid state, and cells that have been induced to exist in a diploid state (e.g., through specific regulation, alteration, inactivation, activation, or modification of meiosis, cytokinesis, or DNA replication). A diploid cell may refer to a cell whose entire genome is diploid, or it may refer to a cell that is diploid in a particular genomic locus of interest.

In some embodiments, the fungal cell is a haploid cell, whose genome is present in one copy. Haploid cells include cells naturally found in a haploid state, or cells that have been induced to exist in a haploid state (e.g., through specific regulation, alteration, inactivation, activation, or modification of meiosis, cytokinesis, or DNA replication). A haploid cell may refer to a cell whose entire genome is haploid, or it may refer to a cell that is haploid in a particular genomic locus of interest.

The compositions and systems, and nucleic acid encoding thereof may be introduced to fungi cells using the delivery systems and methods herein. Examples of delivery systems include lithium acetate treatment, bombardment, electroporation, and those described in Kawai et al., 2010, Bioeng Bugs. 2010 November-December; 1(6): 395-403.

In some examples, a yeast expression vector (e.g., those with one or more regulatory elements) may be used. Examples of such vectors include a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2 μlasmids, yeast integrative plasmids, yeast replicative plasmids, shuttle vectors, and episomal plasmids.

### Biofuel and Materials Production by Fungi

In some embodiments, the compositions, systems, and methods may be used for generating modified fungi for biofuel and material productions. For instance, the modified fungi for production of biofuel or biopolymers from fermentable sugars and optionally to be able to degrade plant-derived lignocellulose derived from agricultural waste as a source of fermentable sugars. Foreign genes required for biofuel production and synthesis may be introduced in to fungi In some examples, the genes may encode enzymes involved in the conversion of pyruvate to ethanol or another product of interest, degrade cellulose (e.g., cellulase), endogenous metabolic pathways which compete with the biofuel production pathway.

In some examples, the compositions, systems, and methods may be used for generating and/or selecting yeast strains with improved xylose or cellobiose utilization, isoprenoid biosynthesis, and/or lactic acid production. One or more genes involved in the metabolism and synthesis of these compounds may be modified and/or introduced to yeast cells. Examples of the methods and genes include lactate dehydrogenase, PDC1 and PDC5, and those described in Ha, S. J., et al. (2011) Proc. Natl. Acad. Sci. USA 108(2):504-9 and Galazka, J. M., et al. (2010) Science 330(6000):84-6; Jakočiūnas T et al., Metab Eng. 2015 March; 28:213-222; Stovicek V, et al., FEMS Yeast Res. 2017 Aug. 1; 17(5).

### Improved Plants and Yeast Cells

The present disclosure further provides improved plants and fungi. The improved and fungi may comprise one or more genes introduced, and/or one or more genes modified by the compositions, systems, and methods herein. The improved plants and fungi may have increased food or feed production (e.g., higher protein, carbohydrate, nutrient or vitamin levels), oil and biofuel production (e.g., methanol, ethanol), tolerance to pests, herbicides, drought, low or high temperatures, excessive water, etc.

The plants or fungi may have one or more parts that are improved, e.g., leaves, stems, roots, tubers, seeds, endosperm, ovule, and pollen. The parts may be viable, nonviable, regeneratable, and/or non-regeneratable.

The improved plants and fungi may include gametes, seeds, embryos, either zygotic or somatic, progeny and/or hybrids of improved plants and fungi. The progeny may be a clone of the produced plant or fungi, or may result from sexual reproduction by crossing with other individuals of the same species to introgress further desirable traits into their offspring. The cell may be in vivo or ex vivo in the cases of multicellular organisms, particularly plants.

### Further Applications of the Crispr-Cas System in Plants

Further applications of the compositions, systems, and methods on plants and fungi include visualization of genetic element dynamics (e.g., as described in Chen B, et al., Cell. 2013 Dec. 19; 155(7):1479-91), targeted gene disruption positive-selection in vitro and in vivo (as described in Malina A et al., Genes Dev. 2013 Dec. 1; 27(23):2602-14), epigenetic modification such as using fusion of Cas and histone-modifying enzymes (e.g., as described in Rusk N, Nat Methods. 2014 January; 11(1):28), identifying transcription regulators (e.g., as described in Waldrip Z. J., Epigenetics. 2014 September; 9(9):1207-11), anti-virus treatment for both RNA and DNA viruses (e.g., as described in Price A. A., et al., Proc Natl Acad Sci USA. 2015 May 12; 112(19):6164-9; Ramanan V et al., Sci Rep. 2015 Jun. 2; 5:10833), alteration of genome complexity such as chromosome numbers (e.g., as described in Karimi-Ashtiyani R et al., Proc Natl Acad Sci USA. 2015 Sep. 8; 112(36):11211-6; Anton T, et al., Nucleus. 2014 March-April; 5(2):163-72), self-cleavage of the CRISPR system for controlled inactivation/activation (e.g., as described Sugano S. S. et al., Plant Cell Physiol. 2014 March; 55(3):475-81), multiplexed gene editing (as described in Kabadi A. M. et al., Nucleic Acids Res. 2014 Oct. 29; 42(19):e147), development of kits for multiplex genome editing (as described in Xing H. L. et al., BMC Plant Biol. 2014 Nov. 29; 14:327), starch production (as described in Hebelstrup K. H. et al., Front Plant Sci. 2015 Apr. 23; 6:247), targeting multiple genes in a family or pathway (e.g., as described in Ma X et al., Mol Plant. 2015 August; 8(8):1274-84), regulation of non-coding genes and sequences (e.g., as described in Lowder L. G., et al., Plant Physiol. 2015 October; 169(2):971-85), editing genes in trees (e.g., as described in Belhaj K et al., Plant Methods. 2013 Oct. 11; 9(1):39; Harrison M M, et al., Genes Dev. 2014 Sep. 1; 28(17):1859-72; Zhou X et al., New Phytol. 2015 October; 208(2):298-301), introduction of mutations for resistance to host-specific pathogens and pests.

Additional examples of modifications of plants and fungi that may be performed using the compositions, systems, and methods include those described in International Patent Publication Nos. WO2016/099887, WO2016/025131, WO2016/073433, WO2017/066175, WO2017/100158, WO 2017/105991, WO2017/106414, WO2016/100272, WO2016/100571, WO 2016/100568, WO 2016/100562, and WO 2017/019867.

### Applications in Non-Human Animals

The compositions, systems, and methods may be used to study and modify non-human animals, e.g., introducing desirable traits and disease resilience, treating diseases, facilitating breeding, etc. In some embodiments, the compositions, systems, and methods may be used to improve breeding and introducing desired traits, e.g., increasing the frequency of trait-associated alleles, introgression of alleles from other breeds/species without linkage drag, and creation of de novo favorable alleles. Genes and other genetic elements that can be targeted may be screened and identified. Examples of application and approaches include those described in Tait-Burkard C, et al., Livestock 2.0—genome editing for fitter, healthier, and more productive farmed animals. Genome Biol. 2018 Nov. 26; 19(1):204; Lillico S, Agricultural applications of genome editing in farmed animals. Transgenic Res. 2019 August; 28(Suppl 2):57-60; Houston R D, et al., Harnessing genomics to fast-track genetic improvement in aquaculture. Nat Rev Genet. 2020 Apr. 16. doi: 10.1038/s41576-020-0227-y, which are incorporated herein by reference in their entireties. Applications described in other sections such as therapeutic, diagnostic, etc. can also be used on the animals herein.

The compositions, systems, and methods may be used on animals such as fish, amphibians, reptiles, mammals, and birds. The animals may be farm and agriculture animals, or pets. Examples of farm and agriculture animals include horses, goats, sheep, swine, cattle, llamas, alpacas, and birds, e.g., chickens, turkeys, ducks, and geese. The animals may be a non-human primate, e.g., baboons, capuchin monkeys, chimpanzees, lemurs, macaques, marmosets, tamarins, spider monkeys, squirrel monkeys, and vervet monkeys. Examples of pets include dogs, cats horses, wolfs, rabbits, ferrets, gerbils, hamsters, chinchillas, fancy rats, guinea pigs, canaries, parakeets, and parrots.

In some embodiments, one or more genes may be introduced (e.g., overexpressed) in the animals to obtain or enhance one or more desired traits. Growth hormones, insulin-like growth factors (IGF-1) may be introduced to increase the growth of the animals, e.g., pigs or salmon (such as described in Pursel V. G. et al., J Reprod Fertil Suppl. 1990; 40:235-45; Waltz E, Nature. 2017; 548:148). Fat-1 gene (e.g., from C elegans) may be introduced for production of larger ratio of n-3 to n-6 fatty acids may be induced, e.g. in pigs (such as described in Li M, et al., Genetics. 2018; 8:1747-54). Phytase (e.g., from E coli) xylanase (e.g., from Aspergillus niger), beta-glucanase (e.g., from bacillus lichenformis) may be introduced to reduce the environmental impact through phosphorous and nitrogen release reduction, e.g. in pigs (such as described in Golovan S P, et al., Nat Biotechnol. 2001; 19:741-5; Zhang X et al., elife. 2018). shRNA decoy may be introduced to induce avian influenza resilience e.g. in chicken (such as described in Lyall et al., Science. 2011; 331:223-6). Lysozyme or lysostaphin may be introduced to induce mastitis resilience e.g., in goat and cow (such as described in Maga E. A. et al., Foodborne Pathog Dis. 2006; 3:384-92; Wall R J, et al., Nat Biotechnol. 2005; 23:445-51). Histone deacetylase such as HDAC6 may be introduced to induce PRRSV resilience, e.g., in pig (such as described in Lu T., et al., PLoS One. 2017; 12:e0169317). CD163 may be modified (e.g., inactivated or removed) to introduce PRRSV resilience in pigs (such as described in Prather R. S. et al., Sci Rep. 2017 Oct. 17; 7(1):13371). Similar approaches may be used to inhibit or remove viruses and bacteria (e.g., Swine Influenza Virus (SIV) strains which include influenza C and the subtypes of influenza A known as H1N1, H1N2, H2N1, H3N1, H3N2, and H2N3, as well as pneumonia, meningitis and oedema) that may be transmitted from animals to humans.

In some embodiments, one or more genes may be modified or edited for disease resistance and production traits. Myostatin (e.g., GDF8) may be modified to increase muscle growth, e.g., in cow, sheep, goat, catfish, and pig (such as described in Crispo M et al., PLoS One. 2015; 10:e0136690; Wang X, et al., Anim Genet. 2018; 49:43-51; Khalil K, et al., Sci Rep. 2017; 7:7301; Kang J-D, et al., RSC Adv. 2017; 7:12541-9). Pc POLLED may be modified to induce horlessness, e.g., in cow (such as described in Carlson D F et al., Nat Biotechnol. 2016; 34:479-81). KISS1R may be modified to induce boretaint (hormone release during sexual maturity leading to undesired meat taste), e.g., in pigs. Dead end protein (dnd) may be modified to induce sterility, e.g., in salmon (such as described in Wargelius A, et al., Sci Rep. 2016; 6:21284). Nano2 and DDX may be modified to induce sterility (e.g., in surrogate hosts), e.g., in pigs and chicken (such as described Park K-E, et al., Sci Rep. 2017; 7:40176; Taylor L et al., Development. 2017; 144:928-34). CD163 may be modified to induce PRRSV resistance, e.g., in pigs (such as described in Whitworth K. M., et al., Nat Biotechnol. 2015; 34:20-2). RELA may be modified to induce ASFV resilience, e.g., in pigs (such as described in Lillico S. G., et al., Sci Rep. 2016; 6:21645). CD18 may be modified to induce Mannheimia (Pasteurella) haemolytica resilience, e.g., in cows (such as described in Shanthalingam S, et al., roc Natl Acad Sci USA. 2016; 113:13186-90). NRAMP1 may be modified to induce tuberculosis resilience, e.g., in cows (such as described in Gao Y et al., Genome Biol. 2017; 18:13). Endogenous retrovirus genes may be modified or removed for xenotransplantation such as described in Yang L, et al. Science. 2015; 350:1101-4; Niu D et al., Science. 2017; 357:1303-7). Negative regulators of muscle mass (e.g., Myostatin) may be modified (e.g., inactivated) to increase muscle mass, e.g., in dogs (as described in Zou Q et al., J Mol Cell Biol. 2015 December; 7(6):580-3).

Animals such as pigs with severe combined immunodeficiency (SCID) may generated (e.g., by modifying RAG2) to provide useful models for regenerative medicine, xenotransplantation (discussed also elsewhere herein), and tumor development. Examples of methods and approaches include those described Lee K, et al., Proc Natl Acad Sci USA. 2014 May 20; 111(20):7260-5; and Schomberg et al. FASEB Journal, April 2016; 30(1):Suppl 571.1.

SNPs in the animals may be modified. Examples of methods and approaches include those described Tan W. et al., Proc Natl Acad Sci USA. 2013 Oct. 8; 110(41):16526-31; Mali P, et al., Science. 2013 Feb. 15; 339(6121):823-6.

Stem cells (e.g., induced pluripotent stem cells) may be modified and differentiated into desired progeny cells, e.g., as described in Heo Y. T. et al., Stem Cells Dev. 2015 Feb. 1; 24(3):393-402.

Profile analysis (such as Igenity) may be performed on animals to screen and identify genetic variations related to economic traits. The genetic variations may be modified to introduce or improve the traits, such as carcass composition, carcass quality, maternal and reproductive traits and average daily gain.

### Therapeutic Uses and Methods of Treatment

Also provided herein are methods of diagnosing, prognosing, treating, and/or preventing a disease, state, or condition in or of a subject. Generally, the methods of diagnosing, prognosing, treating, and/or preventing a disease, state, or condition in or of a subject can include modifying a polynucleotide in a subject or cell thereof using a composition, system, or component thereof described herein and/or include detecting a diseased or healthy polynucleotide in a subject or cell thereof using a composition, system, or component thereof described herein. In some embodiments, the method of treatment or prevention can include using a composition, system, or component thereof to modify a polynucleotide of an infectious organism (e.g. bacterial or virus) within a subject or cell thereof. In some embodiments, the method of treatment or prevention can include using a composition, system, or component thereof to modify a polynucleotide of an infectious organism or symbiotic organism within a subject. The composition, system, and components thereof can be used to develop models of diseases, states, or conditions. The composition, system, and components thereof can be used to detect a disease state or correction thereof, such as by a method of treatment or prevention described herein. The composition, system, and components thereof can be used to screen and select cells that can be used, for example, as treatments or preventions described herein. The composition, system, and components thereof can be used to develop biologically active agents that can be used to modify one or more biologic functions or activities in a subject or a cell thereof.

In general, the method can include delivering a composition, system, and/or component thereof to a subject or cell thereof, or to an infectious or symbiotic organism by a suitable delivery technique and/or composition. Once administered the components can operate as described elsewhere herein to elicit a nucleic acid modification event. In some aspects, the nucleic acid modification event can occur at the genomic, epigenomic, and/or transcriptomic level. DNA and/or RNA cleavage, gene activation, and/or gene deactivation can occur. Additional features, uses, and advantages are described in greater detail below. On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different functional domains to elicit one or more genomic locus events. In addition to treating and/or preventing a disease in a subject, the compositions may be applied in a wide variety of methods for screening in libraries in cells and functional modeling in vivo (e.g. gene activation of lincRNA and identification of function; gain-of-function modeling; loss-of-function modeling; the use the compositions of the invention to establish cell lines and transgenic animals for optimization and screening purposes).

The composition, system, and components thereof described elsewhere herein can be used to treat and/or prevent a disease, such as a genetic and/or epigenetic disease, in a subject. The composition, system, and components thereof described elsewhere herein can be used to treat and/or prevent genetic infectious diseases in a subject, such as bacterial infections, viral infections, fungal infections, parasite infections, and combinations thereof. The composition, system, and components thereof described elsewhere herein can be used to modify the composition or profile of a microbiome in a subject, which can in turn modify the health status of the subject. The composition, system, described herein can be used to modify cells ex vivo, which can then be administered to the subject whereby the modified cells can treat or prevent a disease or symptom thereof. This is also referred to in some contexts as adoptive therapy. The composition, system, described herein can be used to treat mitochondrial diseases, where the mitochondrial disease etiology involves a mutation in the mitochondrial DNA.

Also provided is a method of treating a subject, e.g., a subject in need thereof, comprising inducing gene editing by transforming the subject with the polynucleotide encoding one or more components of the composition, system, or complex or any of polynucleotides or vectors described herein and administering them to the subject. A suitable repair template may also be provided, for example delivered by a vector comprising said repair template. The repair template may be a recombination template herein. Also provided is a method of treating a subject, e.g., a subject in need thereof, comprising inducing transcriptional activation or repression of multiple target gene loci by transforming the subject with the polynucleotides or vectors described herein, wherein said polynucleotide or vector encodes or comprises one or more components of composition, system, complex or component thereof comprising multiple Cas effectors. Where any treatment is occurring ex vivo, for example in a cell culture, then it will be appreciated that the term ‘subject’ may be replaced by the phrase “cell or cell culture.”

Also provided is a method of treating a subject, e.g., a subject in need thereof, comprising inducing gene editing by transforming the subject with the Cas effector(s), advantageously encoding and expressing in vivo the remaining portions of the composition, system, (e.g., RNA, guides). A suitable repair template may also be provided, for example delivered by a vector comprising said repair template. Also provided is a method of treating a subject, e.g., a subject in need thereof, comprising inducing transcriptional activation or repression by transforming the subject with the Cas effector(s) advantageously encoding and expressing in vivo the remaining portions of the composition, system, (e.g., RNA, guides); advantageously in some embodiments the CRISPR enzyme is a catalytically inactive Cas effector and includes one or more associated functional domains. Where any treatment is occurring ex vivo, for example in a cell culture, then it will be appreciated that the term ‘subject’ may be replaced by the phrase “cell or cell culture.”

One or more components of the composition and system described herein can be included in a composition, such as a pharmaceutical composition, and administered to a host individually or collectively. Alternatively, these components may be provided in a single composition for administration to a host. Administration to a host may be performed via viral vectors known to the skilled person or described herein for delivery to a host (e.g. lentiviral vector, adenoviral vector, AAV vector). As explained herein, use of different selection markers (e.g. for lentiviral gRNA selection) and concentration of gRNA (e.g. dependent on whether multiple gRNAs are used) may be advantageous for eliciting an improved effect.

Thus, also described herein are methods of inducing one or more polynucleotide modifications in a eukaryotic or prokaryotic cell or component thereof (e.g. a mitochondria) of a subject, infectious organism, and/or organism of the microbiome of the subject. The modification can include the introduction, deletion, or substitution of one or more nucleotides at a target sequence of a polynucleotide of one or more cell(s). The modification can occur in vitro, ex vivo, in situ, or in vivo.

In some embodiments, the method of treating or inhibiting a condition or a disease caused by one or more mutations in a genomic locus in a eukaryotic organism or a non-human organism can include manipulation of a target sequence within a coding, non-coding or regulatory element of said genomic locus in a target sequence in a subject or a non-human subject in need thereof comprising modifying the subject or a non-human subject by manipulation of the target sequence and wherein the condition or disease is susceptible to treatment or inhibition by manipulation of the target sequence including providing treatment comprising delivering a composition comprising the particle delivery system or the delivery system or the virus particle of any one of the above embodiments or the cell of any one of the above embodiments.

Also provided herein is the use of the particle delivery system or the delivery system or the virus particle of any one of the above embodiments or the cell of any one of the above embodiment in ex vivo or in vivo gene or genome editing; or for use in in vitro, ex vivo or in vivo gene therapy. Also provided herein are particle delivery systems, non-viral delivery systems, and/or the virus particle of any one of the above embodiments or the cell of any one of the above embodiments used in the manufacture of a medicament for in vitro, ex vivo or in vivo gene or genome editing or for use in in vitro, ex vivo or in vivo gene therapy or for use in a method of modifying an organism or a non-human organism by manipulation of a target sequence in a genomic locus associated with a disease or in a method of treating or inhibiting a condition or disease caused by one or more mutations in a genomic locus in a eukaryotic organism or a non-human organism.

In some embodiments, polynucleotide modification can include the introduction, deletion, or substitution of 1-75 nucleotides at each target sequence of said polynucleotide of said cell(s). The modification can include the introduction, deletion, or substitution of at least 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence. The modification can include the introduction, deletion, or substitution of at least 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of at least 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of at least 20, 21, 22, 23, 24, 25, 26, 27, 28, 29, 30, 35, 40, 45, 50, or 75 nucleotides at each target sequence of said cell(s). The modification can include the introduction, deletion, or substitution of at least 40, 45, 50, 75, 100, 200, 300, 400 or 500 nucleotides at each target sequence of said cell(s). The modification can include the introduction, deletion, or substitution of at least 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, or 9900 to 10000 nucleotides at each target sequence of said cell(s).

In some embodiments, the modifications can include the introduction, deletion, or substitution of nucleotides at each target sequence of said cell(s) via nucleic acid components (e.g. guide(s) RNA(s) or sgRNA(s)), such as those mediated by a composition, system, or a component thereof described elsewhere herein. In some embodiments, the modifications can include the introduction, deletion, or substitution of nucleotides at a target or random sequence of said cell(s) via a composition, system, or technique.

In some embodiments, the composition, system, or component thereof can promote Non-Homologous End-Joining (NHEJ). In some embodiments, modification of a polynucleotide by a composition, system, or a component thereof, such as a diseased polynucleotide, can include NHEJ. In some embodiments, promotion of this repair pathway by the composition, system, or a component thereof can be used to target gene or polynucleotide specific knock-outs and/or knock-ins. In some embodiments, promotion of this repair pathway by the composition, system, or a component thereof can be used to generate NHEJ-mediated indels. Nuclease-induced NHEJ can also be used to remove (e.g., delete) sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. The indel can range in size from 1-50 or more base pairs. In some embodiments the indel can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 base pairs or more. If a double-strand break is targeted near to a short target sequence, the deletion mutations caused by the NHEJ repair of ten span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences.

In some embodiments, composition, system, mediated NHEJ can be used in the method to delete small sequence motifs. In some embodiments, composition, system, mediated NHEJ can be used in the method to generate NHEJ-mediate indels that can be targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp). In an embodiment, in which a guide RNA and Cas effector generate a double strand break for the purpose of inducing NHEJ-mediated indels, a guide RNA may be configured to position one double-strand break in close proximity to a nucleotide of the target position. In an embodiment, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position). In an embodiment, in which two guide RNAs complexing with one or more Cas nickases induce two single strand breaks for the purpose of inducing NHEJ-mediated indels, two guide RNAs may be configured to position two single-strand breaks to provide for NHEJ repair a nucleotide of the target position.

For minimization of toxicity and off-target effect, it may be important to control the concentration of Cas mRNA and guide RNA delivered. Optimal concentrations of Cas mRNA and guide RNA can be determined by testing different concentrations in a cellular or non-human eukaryote animal model and using deep sequencing the analyze the extent of modification at potential off-target genomic loci. Alternatively, to minimize the level of toxicity and off-target effect, Cas nickase mRNA (for example S. pyogenes Cas9 with the D10A mutation) can be delivered with a pair of guide RNAs targeting a site of interest. Guide sequences and strategies to minimize toxicity and off-target effects can be as in International Patent Publication No. WO 2014/093622 (PCT/US2013/074667); or, via mutation. Others are as described elsewhere herein.

Typically, in the context of an endogenous CRISPR or system, formation of a CRISPR or complex (comprising a guide sequence hybridized to a target sequence and complexed with one or more Cas proteins) results in cleavage, nicking, and/or another modification of one or both strands in or near (e.g. within 1, 2, 3, 4, 5, 6, 7, 8, 9, 10, 20, 50, or more base pairs from) the target sequence. In some embodiments, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), can also form part of a CRISPR complex, such as by hybridization along at least a portion of the tracr sequence to all or a portion of a tracr mate sequence that is operably linked to the guide sequence.

In some embodiments, a method of modifying a target polynucleotide in a cell to treat or prevent a disease can include allowing a composition, system, or component thereof to bind to the target polynucleotide, e.g., to effect cleavage, nicking, or other modification as the composition, system is capable of said target polynucleotide, thereby modifying the target polynucleotide, wherein the composition, system, or component thereof, complex with a guide sequence, and hybridize said guide sequence to a target sequence within the target polynucleotide, wherein said guide sequence is optionally linked to a tracr mate sequence, which in turn can hybridize to a tracr sequence. In some of these embodiments, the composition, system, or component thereof can be or include a CRISPR-Cas effector complexed with a guide sequence. In some embodiments, modification can include cleaving or nicking one or two strands at the location of the target sequence by one or more components of the composition, system, or component thereof.

The cleavage, nicking, or other modification capable of being performed by the composition, system, can modify transcription of a target polynucleotide. In some embodiments, modification of transcription can include decreasing transcription of a target polynucleotide. In some embodiments, modification can include increasing transcription of a target polynucleotide. In some embodiments, the method includes repairing said cleaved target polynucleotide by homologous recombination with an recombination template polynucleotide, wherein said repair results in a modification such as, but not limited to, an insertion, deletion, or substitution of one or more nucleotides of said target polynucleotide. In some embodiments, said modification results in one or more amino acid changes in a protein expressed from a gene comprising the target sequence. In some embodiments, the modification imparted by the composition, system, or component thereof provides a transcript and/or protein that can correct a disease or a symptom thereof, including but not limited to, any of those described in greater detail elsewhere herein.

In some embodiments, the method of treating or preventing a disease can include delivering one or more vectors or vector systems to a cell, such as a eukaryotic or prokaryotic cell, wherein one or more vectors or vector systems include the composition, system, or component thereof. In some embodiments, the vector(s) or vector system(s) can be a viral vector or vector system, such as an AAV or lentiviral vector system, which are described in greater detail elsewhere herein. In some embodiments, the method of treating or preventing a disease can include delivering one or more viral particles, such as an AAV or lentiviral particle, containing the composition, system, or component thereof. In some embodiments, the viral particle has a tissue specific tropism. In some embodiments, the viral particle has a liver, muscle, eye, heart, pancreas, kidney, neuron, epithelial cell, endothelial cell, astrocyte, glial cell, immune cell, or red blood cell specific tropism.

It will be understood that the composition and system, according to the invention as described herein, such as the composition and system, for use in the methods according to the invention as described herein, may be suitably used for any type of application known for composition, system, preferably in eukaryotes. In certain aspects, the application is therapeutic, preferably therapeutic in a eukaryote organism, such as including but not limited to animals (including human), plants, algae, fungi (including yeasts), etc. Alternatively, or in addition, in certain aspects, the application may involve accomplishing or inducing one or more particular traits or characteristics, such as genotypic and/or phenotypic traits or characteristics, as also described elsewhere herein.

### Treating Diseases of the Circulatory System

In some embodiments, the composition, system, and/or component thereof described herein can be used to treat and/or prevent a circulatory system disease. Exemplary disease is provided, for example. In some embodiments the plasma exosomes of Wahlgren et al. (Nucleic Acids Research, 2012, Vol. 40, No. 17 e130) can be used to deliver the composition, system, and/or component thereof described herein to the blood. In some embodiments, the circulatory system disease can be treated by using a lentivirus to deliver the composition, system, described herein to modify hematopoietic stem cells (HSCs) in vivo or ex vivo (see e.g. Drakopoulou, “Review Article, The Ongoing Challenge of Hematopoietic Stem Cell-Based Gene Therapy for β-Thalassemia,” Stem Cells International, Volume 2011, Article ID 987980, 10 pages, doi:10.4061/2011/987980, which can be adapted for use with the composition, system, herein in view of the description herein). In some embodiments, the circulatory system disorder can be treated by correcting HSCs as to the disease using a composition, system, herein or a component thereof, wherein the composition, system, optionally includes a suitable HDR repair template (see e.g. Cavazzana, “Outcomes of Gene Therapy for β-Thalassemia Major via Transplantation of Autologous Hematopoietic Stem Cells Transduced Ex Vivo with a Lentiviral PA-T87Q-Globin Vector.”; Cavazzana-Calvo, “Transfusion independence and HMGA2 activation after gene therapy of human β-thalassaemia”, Nature 467, 318-322 (16 Sep. 2010) doi:10.1038/nature09328; Nienhuis, “Development of Gene Therapy for Thalassemia, Cold Spring Harbor Perspectives in Medicine, doi: 10.1101/cshperspect.a011833 (2012), LentiGlobin BB305, a lentiviral vector containing an engineered β-globin gene (βA-T87Q); and Xie et al., “Seamless gene correction of β-thalassaemia mutations in patient-specific iPSCs using CRISPR/Cas9 and piggyback” Genome Research gr.173427.114 (2014) www.genome.org/cgi/doi/10.1101/gr.173427.114 (Cold Spring Harbor Laboratory Press; Watts, “Hematopoietic Stem Cell Expansion and Gene Therapy” Cytotherapy 13(10):1164-1171. doi:10.3109/14653249.2011.620748 (2011), which can be adapted for use with the composition, system, herein in view of the description herein). In some embodiments, iPSCs can be modified using a composition, system, described herein to correct a disease polynucleotide associated with a circulatory disease. In this regard, the teachings of Xu et al. (Sci Rep. 2015 Jul. 9; 5:12065. doi: 10.1038/srep12065) and Song et al. (Stem Cells Dev. 2015 May 1; 24(9):1053-65. doi: 10.1089/scd.2014.0347. Epub 2015 Feb. 5) with respect to modifying iPSCs can be adapted for use in view of the description herein with the composition, system, described herein.

The term “Hematopoietic Stem Cell” or “HSC” refers broadly those cells considered to be an HSC, e.g., blood cells that give rise to all the other blood cells and are derived from mesoderm; located in the red bone marrow, which is contained in the core of most bones. HSCs of the invention include cells having a phenotype of hematopoietic stem cells, identified by small size, lack of lineage (lin) markers, and markers that belong to the cluster of differentiation series, like: CD34, CD38, CD90, CD133, CD105, CD45, and also c-kit, -the receptor for stem cell factor. Hematopoietic stem cells are negative for the markers that are used for detection of lineage commitment, and are, thus, called Lin-; and, during their purification by FACS, a number of up to 14 different mature blood-lineage markers, e.g., CD13 & CD33 for myeloid, CD71 for erythroid, CD19 for B cells, CD61 for megakaryocytic, etc. for humans; and, B220 (murine CD45) for B cells, Mac-1 (CD11b/CD18) for monocytes, Gr-1 for Granulocytes, Ter119 for erythroid cells, Il7Ra, CD3, CD4, CD5, CD8 for T cells, etc. Mouse HSC markers: CD34lo/-, SCA-1+, Thy1.1+/lo, CD38+, C-kit+, lin-, and Human HSC markers: CD34+, CD59+, Thy1/CD90+, CD38lo/-, C-kit/CD117+, and lin-. HSCs are identified by markers. Hence in embodiments discussed herein, the HSCs can be CD34+ cells. HSCs can also be hematopoietic stem cells that are CD34-/CD38-. Stem cells that may lack c-kit on the cell surface that are considered in the art as HSCs are within the ambit of the invention, as well as CD133+ cells likewise considered HSCs in the art.

In some embodiments, the treatment or prevention for treating a circulatory system or blood disease can include modifying a human cord blood cell with any modification described herein. In some embodiments, the treatment or prevention for treating a circulatory system or blood disease can include modifying a granulocyte colony-stimulating factor-mobilized peripheral blood cell (mPB) with any modification described herein. In some embodiments, the human cord blood cell or mPB can be CD34+. In some embodiments, the cord blood cell(s) or mPB cell(s) modified can be autologous. In some embodiments, the cord blood cell(s) or mPB cell(s) can be allogenic. In addition to the modification of the disease gene(s), allogenic cells can be further modified using the composition, system, described herein to reduce the immunogenicity of the cells when delivered to the recipient. Such techniques are described elsewhere herein and e.g. Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20 (2010) 857-862, which can be adapted for use with the composition, system, herein. The modified cord blood cell(s) or mPB cell(s) can be optionally expanded in vitro. The modified cord blood cell(s) or mPB cell(s) can be derived to a subject in need thereof using any suitable delivery technique.

The CRISPR-Cas (system may be engineered to target genetic locus or loci in HSCs. In some embodiments, the Cas effector(s) can be codon-optimized for a eukaryotic cell and especially a mammalian cell, e.g., a human cell, for instance, HSC, or iPSC and sgRNA targeting a locus or loci in HSC, such as circulatory disease, can be prepared. These may be delivered via particles. The particles may be formed by the Cas effector protein and the gRNA being admixed. The gRNA and Cas effector protein mixture can be, for example, admixed with a mixture comprising or consisting essentially of or consisting of surfactant, phospholipid, biodegradable polymer, lipoprotein and alcohol, whereby particles containing the gRNA and Cas effector protein may be formed. The invention comprehends so making particles and particles from such a method as well as uses thereof. Particles suitable delivery of the CRISRP-Cas systems in the context of blood or circulatory system or HSC delivery to the blood or circulatory system are described in greater detail elsewhere herein.

In some embodiments, after ex vivo modification the HSCs or iPCS can be expanded prior to administration to the subject. Expansion of HSCs can be via any suitable method such as that described by, Lee, “Improved ex vivo expansion of adult hematopoietic stem cells by overcoming CUL4-mediated degradation of HOXB4.” Blood. 2013 May 16; 121(20):4082-9. doi: 10.1182/blood-2012-09-455204. Epub 2013 Mar. 21.

In some embodiments, the HSCs or iPSCs modified can be autologous. In some embodiments, the HSCs or iPSCs can be allogenic. In addition to the modification of the disease gene(s), allogenic cells can be further modified using the composition, system, described herein to reduce the immunogenicity of the cells when delivered to the recipient. Such techniques are described elsewhere herein and e.g. Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20 (2010) 857-862, which can be adapted for use with the composition, system, herein.

### Treating Neurological Diseases

In some embodiments, the compositions, systems, described herein can be used to treat diseases of the brain and CNS. Delivery options for the brain include encapsulation of CRISPR enzyme and guide RNA in the form of either DNA or RNA into liposomes and conjugating to molecular Trojan horses for trans-blood brain barrier (BBB) delivery. Molecular Trojan horses have been shown to be effective for delivery of B-gal expression vectors into the brain of non-human primates. The same approach can be used to delivery vectors containing CRISPR enzyme and guide RNA. For instance, Xia C F and Boado R. J., Pardridge W M (“Antibody-mediated targeting of siRNA via the human insulin receptor using avidin-biotin technology.” Mol Pharm. 2009 May-June; 6(3):747-51. doi: 10.1021/mp800194) describes how delivery of short interfering RNA (siRNA) to cells in culture, and in vivo, is possible with combined use of a receptor-specific monoclonal antibody (mAb) and avidin-biotin technology. The authors also report that because the bond between the targeting mAb and the siRNA is stable with avidin-biotin technology, and RNAi effects at distant sites such as brain are observed in vivo following an intravenous administration of the targeted siRNA, the teachings of which can be adapted for use with the compositions, systems, herein. In other embodiments, an artificial virus can be generated for CNS and/or brain delivery. See e.g. Zhang et al. (Mol Ther. 2003 January; 7(1):11-8.)), the teachings of which can be adapted for use with the compositions, systems, herein.

### Treating Hearing Diseases

In some embodiments the composition and system described herein can be used to treat a hearing disease or hearing loss in one or both ears. Deafness is of ten caused by lost or damaged hair cells that cannot relay signals to auditory neurons. In such cases, cochlear implants may be used to respond to sound and transmit electrical signals to the nerve cells. But these neurons of ten degenerate and retract from the cochlea as fewer growth factors are released by impaired hair cells.

In some embodiments, the composition, system, or modified cells can be delivered to one or both ears for treating or preventing hearing disease or loss by any suitable method or technique. Suitable methods and techniques include, but are not limited to those set forth in US Patent Publication No. 20120328580 describes injection of a pharmaceutical composition into the ear (e.g., auricular administration), such as into the luminae of the cochlea (e.g., the Scala media, Sc vestibulae, and Sc tympani), e.g., using a syringe, e.g., a single-dose syringe. For example, one or more of the compounds described herein can be administered by intratympanic injection (e.g., into the middle ear), and/or injections into the outer, middle, and/or inner ear; administration in situ, via a catheter or pump (see e.g. McKenna et al., (U.S. Patent Publication No. 2006/0030837) and Jacobsen et al., (U.S. Pat. No. 7,206,639); administration in combination with a mechanical device such as a cochlear implant or a hearing aid, which is worn in the outer ear (see e.g. U.S. Patent Publication No. 2007/0093878, which provides an exemplary cochlear implant suitable for delivery of the compositions, systems, described herein to the ear). Such methods are routinely used in the art, for example, for the administration of steroids and antibiotics into human ears. Injection can be, for example, through the round window of the ear or through the cochlear capsule. Other inner ear administration methods are known in the art (see, e.g., Salt and Plontke, Drug Discovery Today, 10:1299-1306, 2005). In some embodiments, a catheter or pump can be positioned, e.g., in the ear (e.g., the outer, middle, and/or inner ear) of a patient during a surgical procedure. In some embodiments, a catheter or pump can be positioned, e.g., in the ear (e.g., the outer, middle, and/or inner ear) of a patient without the need for a surgical procedure.

In general, the cell therapy methods described in US Patent Publication No. 20120328580 can be used to promote complete or partial differentiation of a cell to or towards a mature cell type of the inner ear (e.g., a hair cell) in vitro. Cells resulting from such methods can then be transplanted or implanted into a patient in need of such treatment. The cell culture methods required to practice these methods, including methods for identifying and selecting suitable cell types, methods for promoting complete or partial differentiation of selected cells, methods for identifying complete or partially differentiated cell types, and methods for implanting complete or partially differentiated cells are described below.

Cells suitable for use in the present invention include, but are not limited to, cells that are capable of differentiating completely or partially into a mature cell of the inner ear, e.g., a hair cell (e.g., an inner and/or outer hair cell), when contacted, e.g., in vitro, with one or more of the compounds described herein. Exemplary cells that are capable of differentiating into a hair cell include, but are not limited to stem cells (e.g., inner ear stem cells, adult stem cells, bone marrow derived stem cells, embryonic stem cells, mesenchymal stem cells, skin stem cells, iPS cells, and fat derived stem cells), progenitor cells (e.g., inner ear progenitor cells), support cells (e.g., Deiters' cells, pillar cells, inner phalangeal cells, tectal cells and Hensen's cells), and/or germ cells. The use of stem cells for the replacement of inner ear sensory cells is described in Li et al., (U.S. Patent Publication No. 2005/0287127) and Li et al., (U.S. patent application Ser. No. 11/953,797). The use of bone marrow derived stem cells for the replacement of inner ear sensory cells is described in Edge et al., PCT/US2007/084654. iPS cells are described, e.g., at Takahashi et al., Cell, Volume 131, Issue 5, Pages 861-872 (2007); Takahashi and Yamanaka, Cell 126, 663-76 (2006); Okita et al., Nature 448, 260-262 (2007); Yu, J. et al., Science 318(5858):1917-1920 (2007); Nakagawa et al., Nat. Biotechnol. 26:101-106 (2008); and Zaehres and Scholer, Cell 131(5):834-835 (2007). Such suitable cells can be identified by analyzing (e.g., qualitatively or quantitatively) the presence of one or more tissue specific genes. For example, gene expression can be detected by detecting the protein product of one or more tissue-specific genes. Protein detection techniques involve staining proteins (e.g., using cell extracts or whole cells) using antibodies against the appropriate antigen. In this case, the appropriate antigen is the protein product of the tissue-specific gene expression. Although, in principle, a first antibody (i.e., the antibody that binds the antigen) can be labeled, it is more common (and improves the visualization) to use a second antibody directed against the first (e.g., an anti-IgG). This second antibody is conjugated either with fluorochromes, or appropriate enzymes for colorimetric reactions, or gold beads (for electron microscopy), or with the biotin-avidin system, so that the location of the primary antibody, and thus the antigen, can be recognized.

The composition and system may be delivered to the ear by direct application of pharmaceutical composition to the outer ear, with compositions modified from US Patent Publication No. 20110142917. In some embodiments the pharmaceutical composition is applied to the ear canal. Delivery to the ear may also be referred to as aural or otic delivery.

In some embodiments, the compositions, systems, or components thereof and/or vectors or vector systems can be delivered to ear via a transfection to the inner ear through the intact round window by a novel proteidic delivery technology which may be applied to the nucleic acid-targeting system of the present invention (see, e.g., Qi et al., Gene Therapy (2013), 1-9). About 40 μl of 10 mM RNA may be contemplated as the dosage for administration to the ear.

According to Rejali et al. (Hear Res. 2007 June; 228(1-2):180-7), cochlear implant function can be improved by good preservation of the spiral ganglion neurons, which are the target of electrical stimulation by the implant and brain derived neurotrophic factor (BDNF) has previously been shown to enhance spiral ganglion survival in experimentally deafened ears. Rejali et al. tested a modified design of the cochlear implant electrode that includes a coating of fibroblast cells transduced by a viral vector with a BDNF gene insert. To accomplish this type of ex vivo gene transfer, Rejali et al. transduced guinea pig fibroblasts with an adenovirus with a BDNF gene cassette insert, and determined that these cells secreted BDNF and then attached BDNF-secreting cells to the cochlear implant electrode via an agarose gel, and implanted the electrode in the scala tympani. Rejali et al. determined that the BDNF expressing electrodes were able to preserve significantly more spiral ganglion neurons in the basal turns of the cochlea after 48 days of implantation when compared to control electrodes and demonstrated the feasibility of combining cochlear implant therapy with ex vivo gene transfer for enhancing spiral ganglion neuron survival. Such a system may be applied to the nucleic acid-targeting system of the present invention for delivery to the ear.

In some embodiments, the system set forth in Mukherjea et al. (Antioxidants & Redox Signaling, Volume 13, Number 5, 2010) can be adapted for transtympanic administration of the composition, system, or component thereof to the ear. In some embodiments, a dosage of about 2 mg to about 4 mg of CRISPR Cas for administration to a human.

In some embodiments, the system set forth in [Jung et al. (Molecular Therapy, vol. 21 no. 4, 834-841 April 2013) can be adapted for vestibular epithelial delivery of the composition, system, or component thereof to the ear. In some embodiments, a dosage of about 1 to about 30 mg of CRISPR Cas for administration to a human.

### Treating Diseases in Non-Dividing Cells

In some embodiments, the gene or transcript to be corrected is in a non-dividing cell. Exemplary non-dividing cells are muscle cells or neurons. Non-dividing (especially non-dividing, fully differentiated) cell types present issues for gene targeting or genome engineering, for example because homologous recombination (HR) is generally suppressed in the G1 cell-cycle phase. However, while studying the mechanisms by which cells control normal DNA repair systems, Durocher discovered a previously unknown switch that keeps HR “of f” in non-dividing cells and devised a strategy to toggle this switch back on. Orthwein et al. (Daniel Durocher's lab at the Mount Sinai Hospital in Ottawa, Canada) recently reported (Nature 16142, published online 9 Dec. 2015) have shown that the suppression of HR can be lifted and gene targeting successfully concluded in both kidney (293T) and osteosarcoma (U20S) cells. Tumor suppressors, BRCA1, PALB2 and BRAC2 are known to promote DNA DSB repair by HR. They found that formation of a complex of BRCA1 with PALB2—BRAC2 is governed by a ubiquitin site on PALB2, such that action on the site by an E3 ubiquitin ligase. This E3 ubiquitin ligase is composed of KEAP1 (a PALB2-interacting protein) in complex with cullin-3 (CUL3)-RBX1. PALB2 ubiquitylation suppresses its interaction with BRCA1 and is counteracted by the deubiquitylase USP11, which is itself under cell cycle control. Restoration of the BRCA1-PALB2 interaction combined with the activation of DNA-end resection is sufficient to induce homologous recombination in G1, as measured by a number of methods including a CRISPR-Cas-based gene-targeting assay directed at USP11 or KEAP1 (expressed from a pX459 vector). However, when the BRCA1-PALB2 interaction was restored in resection-competent G1 cells using either KEAP1 depletion or expression of the PALB2-KR mutant, a robust increase in gene-targeting events was detected. These teachings can be adapted for and/or applied to the Cas compositions, systems, described herein.

Thus, reactivation of HR in cells, especially non-dividing, fully differentiated cell types is preferred, in some embodiments. In some embodiments, promotion of the BRCA1-PALB2 interaction is preferred in some embodiments. In some embodiments, the target ell is a non-dividing cell. In some embodiments, the target cell is a neuron or muscle cell. In some embodiments, the target cell is targeted in vivo. In some embodiments, the cell is in G1 and HR is suppressed. In some embodiments, use of KEAP1 depletion, for example inhibition of expression of KEAP1 activity, is preferred. KEAP1 depletion may be achieved through siRNA, for example as shown in Orthwein et al. Alternatively, expression of the PALB2-KR mutant (lacking all eight Lys residues in the BRCA1-interaction domain is preferred, either in combination with KEAP1 depletion or alone. PALB2-KR interacts with BRCA1 irrespective of cell cycle position. Thus, promotion or restoration of the BRCA1-PALB2 interaction, especially in G1 cells, is preferred in some embodiments, especially where the target cells are non-dividing, or where removal and return (ex vivo gene targeting) is problematic, for example neuron or muscle cells. KEAP1 siRNA is available from ThermoFischer. In some embodiments, a BRCA1-PALB2 complex may be delivered to the G1 cell. In some embodiments, PALB2 deubiquitylation may be promoted for example by increased expression of the deubiquitylase USP11, so it is envisaged that a construct may be provided to promote or up-regulate expression or activity of the deubiquitylase USP11.

### Treating Diseases of the Eye

In some embodiments, the disease to be treated is a disease that affects the eyes. Thus, in some embodiments, the composition, system, or component thereof described herein is delivered to one or both eyes.

The composition, system, can be used to correct ocular defects that arise from several genetic mutations further described in Genetic Diseases of the Eye, Second Edition, edited by Elias I. Traboulsi, Oxford University Press, 2012.

In some embodiments, the condition to be treated or targeted is an eye disorder. In some embodiments, the eye disorder may include glaucoma. In some embodiments, the eye disorder includes a retinal degenerative disease. In some embodiments, the retinal degenerative disease is selected from Stargardt disease, Bardet-Biedl Syndrome, Best disease, Blue Cone Monochromacy, Choroidermia, Cone-rod dystrophy, Congenital Stationary Night Blindness, Enhanced S-Cone Syndrome, Juvenile X-Linked Retinoschisis, Leber Congenital Amaurosis, Malattia Leventinesse, Norrie Disease or X-linked Familial Exudative Vitreoretinopathy, Pattern Dystrophy, Sorsby Dystrophy, Usher Syndrome, Retinitis Pigmentosa, Achromatopsia or Macular dystrophies or degeneration, Retinitis Pigmentosa, Achromatopsia, and age related macular degeneration. In some embodiments, the retinal degenerative disease is Leber Congenital Amaurosis (LCA) or Retinitis Pigmentosa. Other exemplary eye diseases are described in greater detail elsewhere herein.

In some embodiments, the composition, system, is delivered to the eye, optionally via intravitreal injection or subretinal injection. Intraocular injections may be performed with the aid of an operating microscope. For subretinal and intravitreal injections, eyes may be prolapsed by gentle digital pressure and fundi visualized using a contact lens system consisting of a drop of a coupling medium solution on the cornea covered with a glass microscope slide coverslip. For subretinal injections, the tip of a 10-mm 34-gauge needle, mounted on a 5 μl Hamilton syringe may be advanced under direct visualization through the superior equatorial sclera tangentially towards the posterior pole until the aperture of the needle was visible in the subretinal space. Then, 2 μl of vector suspension may be injected to produce a superior bullous retinal detachment, thus confirming subretinal vector administration. This approach creates a self-sealing sclerotomy allowing the vector suspension to be retained in the subretinal space until it is absorbed by the RPE, usually within 48 h of the procedure. This procedure may be repeated in the inferior hemisphere to produce an inferior retinal detachment. This technique results in the exposure of approximately 70% of neurosensory retina and RPE to the vector suspension. For intravitreal injections, the needle tip may be advanced through the sclera 1 mm posterior to the corneoscleral limbus and 2 μl of vector suspension injected into the vitreous cavity. For intracameral injections, the needle tip may be advanced through a corneoscleral limbal paracentesis, directed towards the central cornea, and 2 μl of vector suspension may be injected. For intracameral injections, the needle tip may be advanced through a corneoscleral limbal paracentesis, directed towards the central cornea, and 2 μl of vector suspension may be injected. These vectors may be injected at titers of either 1.0-1.4×10¹⁰ or 1.0-1.4×10⁹ transducing units (TU)/ml.

In some embodiments, for administration to the eye, lentiviral vectors. In some embodiments, the lentiviral vector is an equine infectious anemia virus (EIAV) vector. Exemplary EIAV vectors for eye delivery are described in Balagaan, J Gene Med 2006; 8: 275-285, Published online 21 Nov. 2005 in Wiley InterScience (www.interscience.wiley.com). DOI: 10.1002/jgm.845; Binley et al., HUMAN GENE THERAPY 23:980-991 (September 2012), which can be adapted for use with the composition, system, described herein. In some embodiments, the dosage can be 1.1×10⁵ transducing units per eye (TU/eye) in a total volume of 100 μl.

Other viral vectors can also be used for delivery to the eye, such as AAV vectors, such as those described in Campochiaro et al., Human Gene Therapy 17:167-176 (February 2006), Millington-Ward et al. (Molecular Therapy, vol. 19 no. 4, 642-649 April 2011; Dalkara et al. (Sci Transl Med 5, 189ra76 (2013)), which can be adapted for use with the composition, system, described herein. In some embodiments, the dose can range from about 10⁶ to 10^{9.5} particle units. In the context of the Millington-Ward AAV vectors, a dose of about 2×10¹¹ to about 6×10¹³ virus particles can be administered. In the context of Dalkara vectors, a dose of about 1×10¹⁵ to about 1×10¹⁶ vg/ml administered to a human.

In some embodiments, the sd-rxRNA® system of RXi Pharmaceuticals may be used/and or adapted for delivering composition, system, to the eye. In this system, a single intravitreal administration of 3 μg of sd-rxRNA results in sequence-specific reduction of PPIB mRNA levels for 14 days. The sd-rxRNA® system may be applied to the nucleic acid-targeting system of the present invention, contemplating a dose of about 3 to 20 mg of CRISPR administered to a human.

In other embodiments, the methods of US Patent Publication No. 20130183282, which is directed to methods of cleaving a target sequence from the human rhodopsin gene, may also be modified to the nucleic acid-targeting system of the present invention.

In other embodiments, the methods of US Patent Publication No. 20130202678 for treating retinopathies and sight-threatening ophthalmologic disorders relating to delivering of the Puf-A gene (which is expressed in retinal ganglion and pigmented cells of eye tissues and displays a unique anti-apoptotic activity) to the sub-retinal or intravitreal space in the eye may be used or adapted. In particular, desirable targets are zgc:193933, prdm1a, spata2, tex10, rbb4, ddx3, zp2.2, Blimp-1 and HtrA2, all of which may be targeted by the composition, system, of the present invention.

Wu (Cell Stem Cell, 13:659-62, 2013) designed a guide RNA that led Cas9 to a single base pair mutation that causes cataracts in mice, where it induced DNA cleavage. Then using either the other wild-type allele or oligos given to the zygotes repair mechanisms corrected the sequence of the broken allele and corrected the cataract-causing genetic defect in mutant mouse. This approach can be adapted to and/or applied to the compositions, systems, described herein.

US Patent Publication No. 20120159653, describes use of zinc finger nucleases to genetically modify cells, animals and proteins associated with macular degeneration (MD), the teachings of which can be applied to and/or adapted for the compositions, systems, described herein.

One aspect of US Patent Publication No. 20120159653 relates to editing of any chromosomal sequences that encode proteins associated with MD which may be applied to the nucleic acid-targeting system of the present invention.

### Treating Muscle Diseases and Cardiovascular Diseases

In some embodiments, the composition, system can be used to treat and/or prevent a muscle disease and associated circulatory or cardiovascular disease or disorder. The present invention also contemplates delivering the composition, system, described herein, e.g. Cas effector protein systems, to the heart. For the heart, a myocardium tropic adeno-associated virus (AAVM) is preferred, in particular AAVM41 which showed preferential gene transfer in the heart (see, e.g., Lin-Yanga et al., PNAS, Mar. 10, 2009, vol. 106, no. 10). Administration may be systemic or local. A dosage of about 1-10×10¹⁴ vector genomes are contemplated for systemic administration. See also, e.g., Eulalio et al. (2012) Nature 492: 376 and Somasuntharam et al. (2013) Biomaterials 34: 7790, the teachings of which can be adapted for and/or applied to the compositions, systems, described herein.

For example, US Patent Publication No. 20110023139, the teachings of which can be adapted for and/or applied to the compositions, systems, described herein describes use of zinc finger nucleases to genetically modify cells, animals and proteins associated with cardiovascular disease. Cardiovascular diseases generally include high blood pressure, heart attacks, heart failure, and stroke and TIA. Any chromosomal sequence involved in cardiovascular disease or the protein encoded by any chromosomal sequence involved in cardiovascular disease may be utilized in the methods described in this disclosure. The cardiovascular-related proteins are typically selected based on an experimental association of the cardiovascular-related protein to the development of cardiovascular disease. For example, the production rate or circulating concentration of a cardiovascular-related protein may be elevated or depressed in a population having a cardiovascular disorder relative to a population lacking the cardiovascular disorder. Differences in protein levels may be assessed using proteomic techniques including but not limited to Western blot, immunohistochemical staining, enzyme linked immunosorbent assay (ELISA), and mass spectrometry. Alternatively, the cardiovascular-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR).

The compositions, systems, herein can be used for treating diseases of the muscular system. The present invention also contemplates delivering the composition, system, described herein, effector protein systems, to muscle(s).

In some embodiments, the muscle disease to be treated is a muscle dystrophy such as DMD. In some embodiments, the composition, system, such as a system capable of RNA modification, described herein can be used to achieve exon skipping to achieve correction of the diseased gene. As used herein, the term “exon skipping” refers to the modification of pre-mRNA splicing by the targeting of splice donor and/or acceptor sites within a pre-mRNA with one or more complementary antisense oligonucleotide(s) (AONs). By blocking access of a spliceosome to one or more splice donor or acceptor site, an AON may prevent a splicing reaction thereby causing the deletion of one or more exons from a fully-processed mRNA. Exon skipping may be achieved in the nucleus during the maturation process of pre-mRNAs. In some examples, exon skipping may include the masking of key sequences involved in the splicing of targeted exons by using a composition, system, described herein capable of RNA modification. In some embodiments, exon skipping can be achieved in dystrophin mRNA. In some embodiments, the composition, system, can induce exon skipping at exon 1, 2, 3, 4, 5, 6, 7, 8, 10, 11, 12, 13, 14, 15, 16, 17, 18, 19, 20, 21, 22, 23, 24, 25, 26, 27, 28, 29, 30, 31, 32, 33, 34, 45, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or any combination thereof of the dystrophin mRNA. In some embodiments, the composition, system, can induce exon skipping at exon 43, 44, 50, 51, 52, 55, or any combination thereof of the dystrophin mRNA. Mutations in these exons, can also be corrected using non-exon skipping polynucleotide modification methods.

In some embodiments, for treatment of a muscle disease, the method of Bortolanza et al. Molecular Therapy vol. 19 no. 11, 2055-264 November 2011) may be applied to an AAV expressing CRISPR Cas and injected into humans at a dosage of about 2×10¹⁵ or 2×10¹⁶ vg of vector. The teachings of Bortolanza et al., can be adapted for and/or applied to the compositions, systems, described herein.

In some embodiments, the method of Dumonceaux et al. (Molecular Therapy vol. 18 no. 5, 881-887 May 2010) may be applied to an AAV expressing CRISPR Cas and injected into humans, for example, at a dosage of about 10¹⁴ to about 10¹⁵ vg of vector. The teachings of Dumonceaux described herein can be adapted for and/or applied to the compositions, systems, described herein.

In some embodiments, the method of Kinouchi et al. (Gene Therapy (2008) 15, 1126-1130) may be applied to CRISPR Cas systems described herein and injected into a human, for example, at a dosage of about 500 to 1000 ml of a 40 M solution into the muscle.

In some embodiments, the method of Hagstrom et al. (Molecular Therapy Vol. 10, No. 2, August 2004) can be adapted for and/or applied to the compositions, systems, herein and injected at a dose of about 15 to about 50 mg into the great saphenous vein of a human.

In some embodiments, the method comprises treating a sickle cell related disease, e.g., sickle cell trait, sickle cell disease such as sickle cell anemia, β-thalassaemia. For example, the method and system may be used to modify the genome of the sickle cell, e.g., by correcting one or more mutations of the β-globin gene. In the case of β-thalassaemia, sickle cell anemia can be corrected by modifying HSCs with the systems. The system allows the specific editing of the cell's genome by cutting its DNA and then letting it repair itself. The Cas protein is inserted and directed by a RNA guide to the mutated point and then it cuts the DNA at that point. Simultaneously, a healthy version of the sequence is inserted. This sequence is used by the cell's own repair system to fix the induced cut. In this way, the CRISPR-Cas allows the correction of the mutation in the previously obtained stem cells. The methods and systems may be used to correct HSCs as to sickle cell anemia using a systems that targets and corrects the mutation (e.g., with a suitable HDR template that delivers a coding sequence for β-globin, advantageously non-sickling β-globin); specifically, the guide RNA can target mutation that give rise to sickle cell anemia, and the HDR can provide coding for proper expression of β-globin. An guide RNA that targets the mutation-and-Cas protein containing particle is contacted with HSCs carrying the mutation. The particle also can contain a suitable HDR template to correct the mutation for proper expression of β-globin; or the HSC can be contacted with a second particle or a vector that contains or delivers the HDR template. The so contacted cells can be administered; and optionally treated/expanded; cf. Cartier. The HDR template can provide for the HSC to express an engineered β-globin gene (e.g., PA-T87Q), or β-globin.

### Treating Diseases of the Liver and Kidney

In some embodiments, the composition, system, or component thereof described herein can be used to treat a disease of the kidney or liver. Thus, in some embodiments, delivery of the CRISRP-Cas system or component thereof described herein is to the liver or kidney.

Delivery strategies to induce cellular uptake of the therapeutic nucleic acid include physical force or vector systems such as viral-, lipid- or complex-based delivery, or nanocarriers. From the initial applications with less possible clinical relevance, when nucleic acids were addressed to renal cells with hydrodynamic high-pressure injection systemically, a wide range of gene therapeutic viral and non-viral carriers have been applied already to target posttranscriptional events in different animal kidney disease models in vivo (Csaba Révész and Peter Hamar (2011). Delivery Methods to Target RNAs in the Kidney, Gene Therapy Applications, Prof Chunsheng Kang (Ed.), ISBN: 978-953-307-541-9, InTech, Available from: www.intechopen.com/books/gene-therapy-applications/delivery-methods-to-target-rnas-inthe-kidney). Delivery methods to the kidney may include those in Yuan et al. (Am J Physiol Renal Physiol 295: F605-F617, 2008). The method of Yuang et al. may be applied to the CRISPR Cas system of the present invention contemplating a 1-2 μg subcutaneous injection of CRISPR Cas conjugated with cholesterol to a human for delivery to the kidneys. In some embodiments, the method of Molitoris et al. (J Am Soc Nephrol 20: 1754-1764, 2009) can be adapted to the CRISRP-Cas system of the present invention and a cumulative dose of 12-20 mg/kg to a human can be used for delivery to the proximal tubule cells of the kidneys. In some embodiments, the methods of Thompson et al. (Nucleic Acid Therapeutics, Volume 22, Number 4, 2012) can be adapted to the CRISRP-Cas system of the present invention and a dose of up to 25 mg/kg can be delivered via i.v. administration. In some embodiments, the method of Shimizu et al. (J Am Soc Nephrol 21: 622-633, 2010) can be adapted to the CRISRP-Cas system of the present invention and a dose of about of 10-20 μmol CRISPR Cas complexed with nanocarriers in about 1-2 liters of a physiologic fluid for i.p. administration can be used.

Other various delivery vehicles can be used to deliver the composition, system to the kidney such as viral, hydrodynamic, lipid, polymer nanoparticles, aptamers and various combinations thereof (see e.g. Larson et al., Surgery, (August 2007), Vol. 142, No. 2, pp. (262-269); Hamar et al., Proc Natl Acad Sci, (October 2004), Vol. 101, No. 41, pp. (14883-14888); Zheng et al., Am J Pathol, (October 2008), Vol. 173, No. 4, pp. (973-980); Feng et al., Transplantation, (May 2009), Vol. 87, No. 9, pp. (1283-1289); Q. Zhang et al., PloS ONE, (July 2010), Vol. 5, No. 7, e11709, pp. (1-13); Kushibikia et al., J Controlled Release, (July 2005), Vol. 105, No. 3, pp. (318-331); Wang et al., Gene Therapy, (July 2006), Vol. 13, No. 14, pp. (1097-1103); Kobayashi et al., Journal of Pharmacology and Experimental Therapeutics, (February 2004), Vol. 308, No. 2, pp. (688-693); Wolfrum et al., Nature Biotechnology, (September 2007), Vol. 25, No. 10, pp. (1149-1157); Molitoris et al., J Am Soc Nephrol, (August 2009), Vol. 20, No. 8 pp. (1754-1764); Mikhaylova et al., Cancer Gene Therapy, (March 2011), Vol. 16, No. 3, pp. (217-226); Y. Zhang et al., J Am Soc Nephrol, (April 2006), Vol. 17, No. 4, pp. (1090-1101); Singhal et al., Cancer Res, (May 2009), Vol. 69, No. 10, pp. (4244-4251); Malek et al., Toxicology and Applied Pharmacology, (April 2009), Vol. 236, No. 1, pp. (97-108); Shimizu et al., J Am Soc Nephrology, (April 2010), Vol. 21, No. 4, pp. (622-633); Jiang et al., Molecular Pharmaceutics, (May-June 2009), Vol. 6, No. 3, pp. (727-737); Cao et al, J Controlled Release, (June 2010), Vol. 144, No. 2, pp. (203-212); Ninichuk et al., Am J Pathol, (March 2008), Vol. 172, No. 3, pp. (628-637); Purschke et al., Proc Natl Acad Sci, (March 2006), Vol. 103, No. 13, pp. (5173-5178).

In some embodiments, delivery is to liver cells. In some embodiments, the liver cell is a hepatocyte. Delivery of the composition and system herein may be via viral vectors, especially AAV (and in particular AAV2/6) vectors. These can be administered by intravenous injection. A preferred target for the liver, whether in vitro or in vivo, is the albumin gene. This is a so-called ‘safe harbor” as albumin is expressed at very high levels and so some reduction in the production of albumin following successful gene editing is tolerated. It is also preferred as the high levels of expression seen from the albumin promoter/enhancer allows for useful levels of correct or transgene production (from the inserted recombination template) to be achieved even if only a small fraction of hepatocytes are edited. See sites identified by Wechsler et al. (reported at the 57th Annual Meeting and Exposition of the American Society of Hematology—abstract available online at ash.confex.com/ash/2015/webprogram/Paper86495.html and presented on 6th December 2015) which can be adapted for use with the compositions, systems, herein.

Exemplary liver and kidney diseases that can be treated and/or prevented are described elsewhere herein.

### Treating Epithelial and Lung Diseases

In some embodiments, the disease treated or prevented by the composition and system described herein can be a lung or epithelial disease. The compositions and systems described herein can be used for treating epithelial and/or lung diseases. The present invention also contemplates delivering the composition, system, described herein, to one or both lungs.

In some embodiments, as viral vector can be used to deliver the composition, system, or component thereof to the lungs. In some embodiments, the AAV is an AAV-1, AAV-2, AAV-5, AAV-6, and/or AAV-9 for delivery to the lungs. (see, e.g., Li et al., Molecular Therapy, vol. 17 no. 12, 2067-277 December 2009). In some embodiments, the MOI can vary from 1×10³ to 4×10⁵ vector genomes/cell. In some embodiments, the delivery vector can be an RSV vector as in Zamora et al. (Am J Respir Crit Care Med Vol 183. pp 531-538, 2011. The method of Zamora et al. may be applied to the nucleic acid-targeting system of the present invention and an aerosolized CRISPR Cas, for example with a dosage of 0.6 mg/kg, may be contemplated for the present invention.

Subjects treated for a lung disease may for example receive pharmaceutically effective amount of aerosolized AAV vector system per lung endobronchially delivered while spontaneously breathing. As such, aerosolized delivery is preferred for AAV delivery in general. An adenovirus or an AAV particle may be used for delivery. Suitable gene constructs, each operably linked to one or more regulatory sequences, may be cloned into the delivery vector. In this instance, the following constructs are provided as examples: Cbh or EF1a promoter for Cas, U6 or H1 promoter for guide RNA),: A preferred arrangement is to use a CFTRdelta508 targeting guide, a repair template for deltaF508 mutation and a codon optimized Cas enzyme, with optionally one or more nuclear localization signal or sequence(s) (NLS(s)), e.g., two (2) NLSs.

### Treating Diseases of the Skin

The compositions and systems described herein can be used for the treatment of skin diseases. The present invention also contemplates delivering the composition and system, described herein, to the skin.

In some embodiments, delivery to the skin (intradermal delivery) of the composition, system, or component thereof can be via one or more microneedles or microneedle containing device. For example, in some embodiments the device and methods of Hickerson et al. (Molecular Therapy—Nucleic Acids (2013) 2, e129) can be used and/or adapted to deliver the composition, system, described herein, for example, at a dosage of up to 300 μl of 0.1 mg/ml CRISPR-Cas system to the skin.

In some embodiments, the methods and techniques of Leachman et al. (Molecular Therapy, vol. 18 no. 2, 442-446 February 2010) can be used and/or adapted for delivery of a CIRPSR-Cas system described herein to the skin.

In some embodiments, the methods and techniques of Zheng et al. (PNAS, Jul. 24, 2012, vol. 109, no. 30, 11975-11980) can be used and/or adapted for nanoparticle delivery of a CIRPSR-Cas system described herein to the skin. In some embodiments, as dosage of about 25 nM applied in a single application can achieve gene knockdown in the skin.

### Treating Cancer

The compositions, systems, described herein can be used for the treatment of cancer. The present invention also contemplates delivering the composition, system, described herein, to a cancer cell. Also, as is described elsewhere herein the compositions, systems, can be used to modify an immune cell, such as a CAR or CAR T cell, which can then in turn be used to treat and/or prevent cancer. This is also described in International Patent Publication No. WO 2015/161276, the disclosure of which is hereby incorporated by reference and described herein below.

Target genes suitable for the treatment or prophylaxis of cancer can include those set forth in Tables 10 and 11. In some embodiments, target genes for cancer treatment and prevention can also include those described in International Patent Publication No. WO 2015/048577 the disclosure of which is hereby incorporated by reference and can be adapted for and/or applied to the composition, system, described herein.

### Adoptive Cell Therapy

The compositions, systems, and components thereof described herein can be used to modify cells for an adoptive cell therapy. In an aspect of the invention, methods and compositions which involve editing a target nucleic acid sequence, or modulating expression of a target nucleic acid sequence, and applications thereof in connection with cancer immunotherapy are comprehended by adapting the composition, system, of the present invention. In some examples, the compositions, systems, and methods may be used to modify a stem cell (e.g., induced pluripotent cell) to derive modified natural killer cells, gamma delta T cells, and alpha beta T cells, which can be used for the adoptive cell therapy. In certain examples, the compositions, systems, and methods may be used to modify modified natural killer cells, gamma delta T cells, and alpha beta T cells.

As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: MR1 (see, e.g., Crowther, et al., 2020, Genome-wide CRISPR-Cas9 screening reveals ubiquitous T cell cancer targeting via the monomorphic MHC class I-related protein MR1, Nature Immunology volume 21, pages 178-185), B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J. G., et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gp1OO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX₃); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX₃); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX₅); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR a and R chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO 9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is of ten an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD16, CD22, CD33, CD37, CD64, CD80, CD86, CD134, CD137, CD154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8a transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX_{40/4}-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI la-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO 2014/134165; PCT Publication No. WO 2012/079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIIa, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of. CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139. Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-ζ molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ ID NO: 5246) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY (SEQ ID NO: 5246) and continuing all the way to the carboxy-terminus of the protein. Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in International Patent Publication No. WO 2015/187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signaling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcεRI gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signaling domain as set forth in Table 1 of No. WO 2015/187528. Preferably, the antigen is CD19, more preferably the antigen-binding element is an anti-CD19 scFv, even more preferably the mouse or human anti-CD19 scFv as described in Example 1 of. WO 2015/187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, a chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US Patent Publication Nos. US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (International Patent Publication No. WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In certain embodiments, ACT includes co-transferring CD4+ Th1 cells and CD8+ CTLs to induce a synergistic antitumor response (see, e.g., Li et al., Adoptive cell therapy with CD4+ T helper 1 cells and CD8+ cytotoxic T cells enhances complete rejection of an established tumor, leading to generation of endogenous memory responses to non-targeted tumor epitopes. Clin Transl Immunology. 2017 October; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4+ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells, may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 104-109 cells per kg body weight, preferably 105 to 106 cells/kg body weight including all integer values of cell numbers within those ranges. Dosing in CAR T cell therapies may for example involve administration of from 106 to 109 cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; International Patent Publication WO 2011/146862; International Patent Publication WO 2014/011987; International Patent Publication WO 2013/040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25; 9(374); Legut, et al., 2018, CRISPR-mediated TCR replacement generates superior anticancer transgenic T cells. Blood, 131(3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. The composition and systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see International Patent Publication Nos. WO 2013/176915, WO 2014/059173, WO 2014/172606, WO 2014/184744, and WO 2014/191128).

In certain embodiments, editing may result in inactivation of a gene. By inactivating a gene, it is intended that the gene of interest is not expressed in a functional protein form. In a particular embodiment, the system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that of ten results in changes to the DNA sequence at the site of the cleavage. Repair via non-homologous end joining (NHEJ) of ten results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells, particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells, particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells, particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy?Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

International Patent Publication No. WO 2014/172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX₂, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, International Patent Publication No. WO 2016/196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as the composition or system herein) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, 0-2 microglobulin (B2M) and PD1 simultaneously, to generate gene-disrupted allogeneic CAR T cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, (such as the composition or system herein) (for example, as described in W0201704916).

In certain embodiments, editing of cells, particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in International Patent Publication Nos. WO 2016/011210 and WO 2017/011804).

In certain embodiments, editing of cells, particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiplied edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TTLs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (e.g., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×106/ml. In other embodiments, the concentration used can be from about 1×10⁵/ml to 1×106/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of 1251 labeled 02-microglobulin (02m) into MHC class I/02m/peptide heterotrimeric complexes (see Parker et al., J. Immunol. 152:163, 1994).

In one embodiment, cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™ BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003/057171, U.S. Pat. No. 8,034,334, and U.S. Patent Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in International Patent Publication No. WO 2015/120096, by a method comprising enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO 2015/120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in International Patent Publication No. WO 2017/070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in International Patent Publication No. WO 2016/191756 comprising administering to the patient a dose of cyclophosphamide between 200 mg/m2/day and 2000 mg/m2/day and a dose of fludarabine between 20 mg/m2/day and 900 mg/m²/day.

### Diseases

Genetic Diseases and Diseases with a Genetic and/or Epigenetic Aspect

The compositions, systems, or components thereof can be used to treat and/or prevent a genetic disease or a disease with a genetic and/or epigenetic aspect. The genes and conditions exemplified herein are not exhaustive. In some embodiments, a method of treating and/or preventing a genetic disease can include administering a composition, system, and/or one or more components thereof to a subject, where the composition, system, and/or one or more components thereof is capable of modifying one or more copies of one or more genes associated with the genetic disease or a disease with a genetic and/or epigenetic aspect in one or more cells of the subject. In some embodiments, modifying one or more copies of one or more genes associated with a genetic disease or a disease with a genetic and/or epigenetic aspect in the subject can eliminate a genetic disease or a symptom thereof in the subject. In some embodiments, modifying one or more copies of one or more genes associated with a genetic disease or a disease with a genetic and/or epigenetic aspect in the subject can decrease the severity of a genetic disease or a symptom thereof in the subject. In some embodiments, the compositions, systems, or components thereof can modify one or more genes or polynucleotides associated with one or more diseases, including genetic diseases and/or those having a genetic aspect and/or epigenetic aspect, including but not limited to, any one or more set forth in Table 10. It will be appreciated that those diseases and associated genes listed herein are non-exhaustive and non-limiting. Further some genes play roles in the development of multiple diseases.

TABLE 10
Exemplary Genetic and Other Diseases and Associated Genes

| | Primary | | |
|---|---|---|---|
| | Tissues or | Additional | |
| | System | Tissues/Systems | |
| Disease Name | Affected | Affected | Genes |
| Achondroplasia | Bone and | | fibroblast growth factor receptor 3 |
| | Muscle | | (FGFR3) |
| Achromatopsia | eye | | CNGA3, CNGB3, GNAT2, PDE6C, |
| | | | PDE6H, ACHM2, ACHM3, |
| Acute Renal Injury | kidney | | NFkappaB, AATF, p85alpha, FAS, |
| | | | Apoptosis cascade elements (e.g. |
| | | | FASR, Caspase 2, 3, 4, 6, 7, 8, 9, 10, |
| | | | AKT, TNF alpha, IGF1, IGF1R, |
| | | | RIPK1), p53 |
| Age Related Macular | eye | | Abcr; CCL2; CC2; CP |
| Degeneration | | | (ceruloplasmin); Timp3; cathepsinD; |
| | | | VLDLR, CCR2 |
| AIDS | Immune System | | KIR3DL1, NKAT3, NKB1, AMB11, |
| | | | KIR3DS1, IFNG, CXCL12, SDF1 |
| Albinism (including | Skin, hair, eyes, | | TYR, OCA2, TYRP1, and SLC45A2, |
| oculocutaneous albinism (types | | | SLC24A5 and C10orf11 |
| 1-7) and ocular albinism) | | | |
| Alkaptonuria | Metabolism of | Tissues/organs | HGD |
| | amino acids | where | |
| | | homogentisic | |
| | | acid | |
| | | accumulates, | |
| | | particularly | |
| | | cartilage (joints), | |
| | | heart valves, | |
| | | kidneys | |
| alpha-1 antitrypsin deficiency | Lung | Liver, skin, | SERPINA1, those set forth in |
| (AATD or A1AD) | | vascular system, | WO2017165862, PiZ allele |
| | | kidneys, GI | |
| ALS | CNS | | SOD1; ALS2; ALS3; ALS5; |
| | | | ALS7; STEX; FUS; TARDBP; VEGF |
| | | | (VEGF-a; |
| | | | VEGF-b; VEGF-c); DPP6; NEFH, |
| | | | PTGS1, SLC1A2, TNFRSF10B, |
| | | | PRPH, HSP90AA1, CRIA2, IFNG, |
| | | | AMPA2 S100B, FGF2, AOX1, CS, |
| | | | TXN, RAPHJ1, MAP3K5, NBEAL1, |
| | | | GPX1, ICA1L, RAC1, MAPT, ITPR2, |
| | | | ALS2CR4, GLS, ALS2CR8, CNTFR, |
| | | | ALS2CR11, FOLH1, FAM117B, |
| | | | P4HB, CNTF, SQSTM1, STRADB, |
| | | | NAIP, NLR, YWHAQ, SLC33A1, |
| | | | TRAK2, SCA1, NIF3L1, NIF3, |
| | | | PARD3B, COX8A, CDK15, HECW1, |
| | | | HECT, C2, WW 15, NOS1, MET, |
| | | | SOD2, HSPB1, NEFL, CTSB, ANG, |
| | | | HSPA8, RNase A, VAPB, VAMP, |
| | | | SNCA, alpha HGF, CAT, ACTB, |
| | | | NEFM, TH, BCL2, FAS, CASP3, |
| | | | CLU, SMN1, G6PD, BAX, HSF1, |
| | | | RNF19A, JUN, ALS2CR12, HSPA5, |
| | | | MAPK14, APEX1, TXNRD1, NOS2, |
| | | | TIMP1, CASP9, XIAP, GLG1, EPO, |
| | | | VEGFA, ELN, GDNF, NFE2L2, |
| | | | SLC6A3, HSPA4, APOE, PSMB8, |
| | | | DCTN2, TIMP3, KIFAP3, SLC1A1, |
| | | | SMN2, CCNC, STUB1, ALS2, |
| | | | PRDX6, SYP, CABIN1, CASP1, |
| | | | GART, CDK5, ATXN3, RTN4, |
| | | | C1QB, VEGFC, HTT, PARK7, XDH, |
| | | | GFAP, MAP2, CYCS, FCGR3B, CCS, |
| | | | UBL5, MMP9m SLC18A3, TRPM7, |
| | | | HSPB2, AKT1, DEERL1, CCL2, |
| | | | NGRN, GSR, TPPP3, APAF1, |
| | | | BTBD10, GLUD1, CXCR4, S:C1A3, |
| | | | FLT1, PON1, AR, LIF, ERBB3, |
| | | | :GA:S1, CD44, TP53, TLR3, GRIA1, |
| | | | GAPDH, AMP A, GRIK1, DES, |
| | | | CHAT, FLT4, CHMP2B, BAG1, |
| | | | CHRNA4, GSS, BAK1, KDR, GSTP1, |
| | | | OGG1, IL6 |
| Alzheimer’s Disease | Brain | | E1; CHIP; UCH; UBB; Tan; LRP; |
| | | | PICALM; CLU; PS1; |
| | | | SORL1; CR1; VLDLR; UBA1; |
| | | | UBA3; CHIP28; AQP1; UCHL1; |
| | | | UCHL3; APP, AAA, CVAP, AD1, |
| | | | APOE, AD2, DCP1, ACE1, MPO, |
| | | | PACIP1, PAXIP1L, PTIP, A2M, |
| | | | BLMH, BMH, PSEN1, AD3, ALAS2, |
| | | | ABCA1, BIN1, BDNF, BTNL8, |
| | | | C1ORF49, CDH4, CHRNB2, |
| | | | CKLFSF2, CLEC4E, CR1L, CSF3R, |
| | | | CST3, CYP2C, DAPK1, ESR1, |
| | | | FCAR, FCGR3B, FFA2, FGA, GAB2, |
| | | | GALP, GAPDHS, GMPB, HP, HTR7, |
| | | | IDE, IF127, IFI6, IFIT2, IL1RN, IL- |
| | | | 1RA, IL8RA, IL8RB, JAG1, KCNJ15, |
| | | | LRP6, MAPT, MARK4, MPHOSPH1, |
| | | | MTHFR, NBN, NCSTN, NIACR2, |
| | | | NMNAT3, NTM, ORM1, P2RY13, |
| | | | PBEF1, PCK1, PICALM, PLAU, |
| | | | PLXNC1, PRNP, PSEN1, PSEN2, |
| | | | PTPRA, RALGPS2, RGSL2, |
| | | | SELENBP1, SLC25A37, SORL1, |
| | | | Mitoferrin-1, TF, TFAM, TNF, |
| | | | TNFRSF10C, UBE1C |
| Amyloidosis | | | APOA1, APP, AAA, CVAP, AD1, |
| | | | GSN, FGA, LYZ, TTR, PALB |
| Amyloid neuropathy | | | TTR, PALB |
| Anemia | Blood | | CDAN1, CDA1, RPS19, DBA, PKLR, |
| | | | PK1, NT5C3, UMPH1, PSN1, RHAG, |
| | | | RH50A, NRAMP2, SPTB, ALAS2, |
| | | | ANH1, ASB, ABCB7, ABC7, ASAT |
| Angelman Syndrome | Nervous system, | | UBE3A |
| | brain | | |
| Attention Deficit Hyperactivity | Brain | | PTCHD1 |
| Disorder (ADHD) | | | |
| Autoimmune lymphoproliferative | Immune system | | TNFRSF6, APT1, FAS, CD95, |
| syndrome | | | ALPS1A |
| Autism, Autism spectrum | Brain | | PTCHD1; Mecp2; BZRAP1; MDGA2; |
| disorders (ASDs), including | | | Sema5A; Neurexin 1; GLO1, RTT, |
| Asperger's and a general | | | PPMX, MRX16, RX79, NLGN3, |
| diagnostic category called | | | NLGN4, KIAA1260, AUTSX2, |
| Pervasive Developmental | | | FMR1, FMR2; FXR1; FXR2; |
| Disorders (PDDs) | | | MGLUR5, ATP10C, CDH10, GRM6, |
| | | | MGLUR6, CDH9, CNTN4, NLGN2, |
| | | | CNTNAP2, SEMA5A, DHCR7, |
| | | | NLGN4X, NLGN4Y, DPP6, NLGN5, |
| | | | EN2, NRCAM, MDGA2, NRXN1, |
| | | | FMR2, AFF2, FOXP2, OR4M2, |
| | | | OXTR, FXR1, FXR2, PAH, |
| | | | GABRA1, PTEN, GABRA5, PTPRZ1, |
| | | | GABRB3, GABRG1, HIRIP3, |
| | | | SEZ6L2, HOXA1, SHANK3, IL6, |
| | | | SHBZRAP1, LAMB1, SLC6A4, |
| | | | SERT, MAPK3, TAS2R1, MAZ, |
| | | | TSC1, MDGA2, TSC2, MECP2, |
| | | | UBE3A, WNT2, see also |
| | | | 20110023145 |
| autosomal dominant polycystic | kidney | liver | PKD1, PKD2 |
| kidney disease (ADPKD)- | | | |
| (includes diseases such as von | | | |
| Hippel-Lindau disease and | | | |
| tuberous sclerosis complex | | | |
| disease) | | | |
| Autosomal Recessive Polycystic | kidney | liver | PKDH1 |
| Kidney Disease (ARPKD) | | | |
| Ataxia-Telangiectasia (a.k.a | Nervous system, | various | ATM |
| Louis Bar syndrome) | immune system | | |
| B-Cell Non-Hodgkin Lymphoma | | | BCL7A, BCL7 |
| Bardet-Biedl syndrome | Eye, | Liver, ear, | ARL6, BBS1, BBS2, BBS4, BBS5, |
| | musculoskeletal | gastrointestinal | BBS7, BBS9, BBS10, BBS12, |
| | system, kidney, | system, brain | CEP290, INPP5E, LZTFL1, MKKS, |
| | reproductive | | MKS1, SDCCAG8, TRIM32, TTC8 |
| | organs | | |
| Bare Lymphocyte Syndrome | blood | | TAPBP, TPSN, TAP2, ABCB3, PSF2, |
| | | | RING11, MHC2TA, C2TA, RFX5, |
| | | | RFXAP, RFX5 |
| Bartter’s Syndrome (types I, II, | kidney | | SLC12A1 (type I), KCNJ1 (type II), |
| III, IVA and B, and V) | | | CLCNKB (type III), BSND (type IV |
| | | | A), or both the CLCNKA CLCNKB |
| | | | genes (type IV B), CASR (type V). |
| Becker muscular dystrophy | Muscle | | DMD, BMD, MYF6 |
| Best Disease (Vitelliform | eye | | VMD2 |
| Macular Dystrophy type 2) | | | |
| Bleeding Disorders | blood | | TBXA2R, P2RX1, P2X1 |
| Blue Cone Monochromacy | eye | | OPN1LW, OPN1MW, and LCR |
| Breast Cancer | Breast tissue | | BRCA1, BRCA2, COX-2 |
| Bruton's Disease (aka X-linked | Immune system, | | BTK |
| Agammglobulinemia) | specifically B | | |
| | cells | | |
| Cancers (e.g., lymphoma, chronic | Various | | FAS, BID, CTLA4, PDCD1, CBLB, |
| lymphocytic leukemia (CLL), B | | | PTPN6, TRAC, TRBC, those |
| cell acute lymphocytic leukemia | | | described in WO2015048577 |
| (B-ALL), acute lymphoblastic | | | |
| leukemia, acute myeloid | | | |
| leukemia, non-Hodgkin's | | | |
| lymphoma (NHL), diffuse large | | | |
| cell lymphoma (DLCL), multiple | | | |
| myeloma, renal cell carcinoma | | | |
| (RCC), neuroblastoma, colorectal | | | |
| cancer, breast cancer, ovarian | | | |
| cancer, melanoma, sarcoma, | | | |
| prostate cancer, lung cancer, | | | |
| esophageal cancer, hepatocellular | | | |
| carcinoma, pancreatic cancer, | | | |
| astrocytoma, mesothelioma, head | | | |
| and neck cancer, and | | | |
| medulloblastoma | | | |
| Cardiovascular Diseases | heart | Vascular system | IL1B, XDH, TP53, PTGS, MB, IL4, |
| | | | ANGPT1, ABCGu8, CTSK, PTGIR, |
| | | | KCNJ11, INS, CRP, PDGFRB, |
| | | | CCNA2, PDGFB, KCNJ5, KCNN3, |
| | | | CAPN10, ADRA2B, ABCG5, |
| | | | PRDX2, CPAN5, PARP14, MEX3C, |
| | | | ACE, RNF, IL6, TNF, STN, |
| | | | SERPINE1, ALB, ADIPOQ, APOB, |
| | | | APOE, LEP, MTHFR, APOA1, |
| | | | EDN1, NPPB, NOS3, PPARG, PLAT, |
| | | | PTGS2, CETP, AGTR1, HMGCR, |
| | | | IGF1, SELE, REN, PPARA, PON1, |
| | | | KNG1, CCL2, LPL, VWF, F2, |
| | | | ICAM1, TGFB, NPPA, IL10, EPO, |
| | | | SOD1, VCAM1, IFNG, LPA, MPO, |
| | | | ESR1, MAPK, HP, F3, CST3, COG2, |
| | | | MMP9, SERPINC1, F8, HMOX1, |
| | | | APOC3, IL8, PROL1, CBS, NOS2, |
| | | | TLR4, SELP, ABCA1, AGT, LDLR, |
| | | | GPT, VEGFA, NR3C2, IL18, NOS1, |
| | | | NR3C1, FGB, HGF, IL1A, AKT1, |
| | | | LIPC, HSPD1, MAPK14, SPP1, |
| | | | ITGB3, CAT, UTS2, THBD, F10, CP, |
| | | | TNFRSF11B, EGFR, MMP2, PLG, |
| | | | NPY, RHOD, MAPK8, MYC, FN1, |
| | | | CMA1, PLAU, GNB3, ADRB2, |
| | | | SOD2, F5, VDR, ALOX5, HLA- |
| | | | DRB1, PARP1, CD40LG, PON2, |
| | | | AGER, IRS1, PTGS1, ECE1, F7, |
| | | | IRMN, EPHX2, IGFBP1, MAPK10, |
| | | | FAS, ABCB1, JUN, IGFBP3, CD14, |
| | | | PDE5A, AGTR2, CD40, LCAT, |
| | | | CCR5, MMP1, TIMP1, ADM, |
| | | | DYT10, STAT3, MMP3, ELN, USF1, |
| | | | CFH, HSPA4, MMP12, MME, F2R, |
| | | | SELL, CTSB, ANXA5, ADRB1, |
| | | | CYBA, FGA, GGT1, LIPG, HIF1A, |
| | | | CXCR4, PROC, SCARB1, CD79A, |
| | | | PLTP, ADD1, FGG, SAA1, KCNH2, |
| | | | DPP4, NPR1, VTN, KIAA0101, FOS, |
| | | | TLR2, PPIG, IL1R1, AR, CYP1A1, |
| | | | SERPINA1, MTR, RBP4, APOA4, |
| | | | CDKN2A, FGF2, EDNRB, ITGA2, |
| | | | VLA-2, CABIN1, SHBG, HMGB1, |
| | | | HSP90B2P, CYP3A4, GJA1, CAV1, |
| | | | ESR2, LTA, GDF15, BDNF, |
| | | | CYP2D6, NGF, SP1, TGIF1, SRC, |
| | | | EGF, PIK3CG, HLA-A, KCNQ1, |
| | | | CNR1, FBN1, CHKA, BEST1, |
| | | | CTNNB1, IL2, CD36, PRKAB1, TPO, |
| | | | ALDH7A1, CX3CR1, TH, F9, CH1, |
| | | | TF, HFE, IL17A, PTEN, GSTM1, |
| | | | DMD, GATA4, F13A1, TTR, FABP4, |
| | | | PON3, APOC1, INSR, TNFRSF1B, |
| | | | HTR2A, CSF3, CYP2C9, TXN, |
| | | | CYP11B2, PTH, CSF2, KDR, |
| | | | PLA2G2A, THBS1, GCG, RHOA, |
| | | | ALDH2, TCF7L2, NFE2L2, |
| | | | NOTCH1, UGT1A1, IFNA1, PPARD, |
| | | | SIRT1, GNHR1, PAPPA, ARR3, |
| | | | NPPC, AHSP, PTK2, IL13, MTOR, |
| | | | ITGB2, GSTT1, IL6ST, CPB2, |
| | | | CYP1A2, HNF4A, SLC64A, |
| | | | PLA2G6, TNFSF11, SLC8A1, F2RL1, |
| | | | AKR1A1, ALDH9A1, BGLAP, |
| | | | MTTP, MTRR, SULT1A3, RAGE, |
| | | | C4B, P2RY12, RNLS, CREB1, |
| | | | POMC, RAC1, LMNA, CD59, |
| | | | SCM5A, CYP1B1, MIF, MMP13, |
| | | | TIMP2, CYP19A1, CUP21A2, |
| | | | PTPN22, MYH14, MBL2, SELPLG, |
| | | | AOC3, CTSL1, PCNA, IGF2, ITGB1, |
| | | | CAST, CXCL12, IGHE, KCNE1, |
| | | | TFRC, COL1A1, COL1A2, IL2RB, |
| | | | PLA2G10, ANGPT2, PROCR, NOX4, |
| | | | HAMP, PTPN11, SLCA1, IL2RA, |
| | | | CCL5, IRF1, CF:AR, CA:CA, EIF4E, |
| | | | GSTP1, JAK2, CYP3A5, HSPG2, |
| | | | CCL3, MYD88, VIP, SOAT1, |
| | | | ADRBK1, NR4A2, MMP8, NPR2, |
| | | | GCH1, EPRS, PPARGC1A, F12, |
| | | | PECAM1, CCL4, CERPINA34, |
| | | | CASR, FABP2, TTF2, PROS1, CTF1, |
| | | | SGCB, YME1L1, CAMP, ZC3H12A, |
| | | | AKR1B1, MMP7, AHR, CSF1, |
| | | | HDAC9, CTGF, KCNMA1, UGT1A, |
| | | | PRKCA, COMT, S100B, EGR1, PRL, |
| | | | IL15, DRD4, CAMK2G, SLC22A2, |
| | | | CCL11, PGF, THPO, GP6, TACR1, |
| | | | NTS, HNF1A, SST, KCDN1, |
| | | | LOC646627, TBXAS1, CUP2J2, |
| | | | TBXA2R, ADH1C, ALOX12, AHSG, |
| | | | BHMT, GJA4, SLC25A4, ACLY, |
| | | | ALOX5AP, NUMA1, CYP27B1, |
| | | | CYSLTR2, SOD3, LTC4S, UCN, |
| | | | GHRL, APOC2, CLEC4A, |
| | | | KBTBD10, TNC, TYMS, SHC1, |
| | | | LRP1, SOCS3, ADH1B, KLK3, |
| | | | HSD11B1, VKORC1, SERPINB2, |
| | | | TNS1, RNF19A, EPOR, ITGAM, |
| | | | PITX2, MAPK7, FCGR3A, LEEPR, |
| | | | ENG, GPX1, GOT2, HRH1, NR112, |
| | | | CRH, HTR1A, VDAC1, HPSE, |
| | | | SFTPD, TAP2, RMF123, PTK2Bm |
| | | | NTRK2, IL6R, ACHE, GLP1R, GHR, |
| | | | GSR, NQO1, NR5A1, GJB2, |
| | | | SLC9A1, MAOA, PCSK9, FCGR2A, |
| | | | SERPINF1, EDN3, UCP2, TFAP2A, |
| | | | C4BPA, SERPINF2, TYMP, ALPP, |
| | | | CXCR2, SLC3A3, ABCG2, ADA, |
| | | | JAK3, HSPA1A, FASN, FGF1, F11, |
| | | | ATP7A, CR1, GFPA, ROCK1, |
| | | | MECP2, MYLK, BCHE, LIPE, |
| | | | ADORA1, WRN, CXCR3, CD81, |
| | | | SMAD7, LAMC2, MAP3K5, CHGA, |
| | | | IAPP, RHO, ENPP1, PTHLH, NRG1, |
| | | | VEGFC, ENPEP, CEBPB, NAGLU,. |
| | | | F2RL3, CX3CL1, BDKRB1, |
| | | | ADAMTS13, ELANE, ENPP2, CISH, |
| | | | GAST, MYOC, ATP1A2, NF1, GJB1, |
| | | | MEF2A, VCL, BMPR2, TUBB, |
| | | | CDC42, KRT18, HSF1, MYB, |
| | | | PRKAA2, ROCK2, TFP1, PRKG1, |
| | | | BMP2, CTNND1, CTH, CTSS, |
| | | | VAV2, NPY2R, IGFBP2, CD28, |
| | | | GSTA1, PPIA, APOH, S100A8, IL11, |
| | | | ALOX15, FBLN1, NR1H3, SCD, GIP, |
| | | | CHGB, PRKCB, SRD5A1, HSD11B2, |
| | | | CALCRL, GALNT2, ANGPTL4, |
| | | | KCNN4, PIK3C2A, HBEGF, |
| | | | CYP7A1, HLA-DRB5, BNIP3, |
| | | | GCKR, S100A12, PADI4, HSPA14, |
| | | | CXCR1, H19, KRTAP19-3, IDDM2, |
| | | | RAC2, YRY1, CLOCK, NGFR, DBH, |
| | | | CHRNA4, CACNA1C, PRKAG2, |
| | | | CHAT, PTGDS, NR1H2, TEK, |
| | | | VEGFB, MEF2C, MAPKAPK2, |
| | | | TNFRSF11A, HSPA9, CYSLTR1, |
| | | | MAT1A, OPRL1, IMPA1, CLCN2, |
| | | | DLD, PSMA6, PSMB8, CHI3L1, |
| | | | ALDH1B1, PARP2, STAR, LBP, |
| | | | ABCC6, RGS2, EFNB2, GJB6, |
| | | | APOA2, AMPD1, DYSF, |
| | | | FDFT1, EMD2, CCR6, GJB3, IL1RL1, |
| | | | ENTPD1, BBS4, CELSR2, F11R, |
| | | | RAPGEF3, HYAL1, ZNF259, |
| | | | ATOX1, ATF6, KHK, SAT1, GGH, |
| | | | TIMP4, SLC4A4, PDE2A, PDE3B, |
| | | | FADS1, FADS2, TMSB4X, TXNIP, |
| | | | LIMS1, RHOB, LY96, FOXO1, |
| | | | PNPLA2, TRH, GJC1, S:C17A5, FTO, |
| | | | GJD2, PRSC1, CASP12, GPBAR1, |
| | | | PXK, IL33, TRIB1, PBX4, NUPR1, |
| | | | 15-SEP, CILP2, TERC, GGT2, |
| | | | MTCO1, UOX, AVP |
| Cataract | eye | | CRYAA, CRYA1, CRYBB2, CRYB2, |
| | | | PITX3, BFSP2, CP49, CP47, CRYAA, |
| | | | CRYA1, PAX6, AN2, MGDA, |
| | | | CRYBA1, CRYB1, CRYGC, CRYG3, |
| | | | CCL, LIM2, MP19, CRYGD, CRYG4, |
| | | | BFSP2, CP49, CP47, HSF4, CTM, |
| | | | HSF4, CTM, MIP, AQP0, CRYAB, |
| | | | CRYA2, CTPP2, CRYBB1, CRYGD, |
| | | | CRYG4, CRYBB2, CRYB2, CRYGC, |
| | | | CRYG3, CCL, CRYAA, CRYA1, |
| | | | GJA8, CX50, CAE1, GJA3, CX46, |
| | | | CZP3, CAE3, CCM1, CAM, KRIT1 |
| CDKL-5 Deficiencies or | Brain, CNS | | CDKL5 |
| Mediated Diseases | | | |
| Charcot-Marie-Tooth (CMT) | Nervous system | Muscles | PMP22 (CMT1A and E), MPZ |
| disease (Types 1, 2, 3, 4,) | | (dystrophy) | (CMT1B), LITAF (CMT1C), EGR2 |
| | | | (CMT1D), NEFL (CMT1F), GJB1 |
| | | | (CMT1X), MFN2 (CMT2A), KIF1B |
| | | | (CMT2A2B), RAB7A (CMT2B), |
| | | | TRPV4 (CMT2C), GARS (CMT2D), |
| | | | NEFL (CMT2E), GAPD1 (CMT2K), |
| | | | HSPB8 (CMT2L), DYNC1H1, |
| | | | CMT2O), LRSAM1 (CMT2P), |
| | | | IGHMBP2 (CMT2S), MORC2 |
| | | | (CMT2Z), GDAP1 (CMT4A), |
| | | | MTMR2 or SBF2/MTMR13 |
| | | | (CMT4B), SH3TC2 (CMT4C), |
| | | | NDRG1 (CMT4D), PRX (CMT4F), |
| | | | FIG4 (CMT4J), NT-3 |
| Chédiak-Higashi Syndrome | Immune system | Skin, hair, eyes, | LYST |
| | | neurons | |
| Choroidermia | | | CHM, REP1, |
| Chorioretinal atrophy | eye | | PRDM13, RGR, TEAD1 |
| Chronic Granulomatous Disease | Immune system | | CYBA, CYBB, NCF1, NCF2, NCF4 |
| Chronic Mucocutaneous | Immune system | | AIRE, CARD9, CLEC7A IL12B, |
| Candidiasis | | | IL12B1, IL1F, IL17RA, IL17RC, |
| | | | RORC, STAT1, STAT3, TRAF31P2 |
| Cirrhosis | liver | | KRT18, KRT8, CIRH1A, NAIC, |
| | | | TEX292, KIAA1988 |
| Colon cancer (Familial | Gastrointestinal | | FAP: APC HNPCC: |
| adenomatous polyposis (FAP) | | | MSH2, MLH1, PMS2, SH6, PMS1 |
| and hereditary nonpolyposis | | | |
| colon cancer (HNPCC)) | | | |
| Combined Immunodeficiency | Immune System | | IL2RG, SCIDX1, SCIDX, IMD4); |
| | | | HIV-1 (CCL5, SCYA5, D17S136E, |
| | | | TCP228 |
| Cone(-rod) dystrophy | eye | | AIPL1, CRX, GUA1A, GUCY2D, |
| | | | PITPM3, PROM1, PRPH2, RIMS1, |
| | | | SEMA4A, ABCA4, ADAM9, ATF6, |
| | | | C21ORF2, C8ORF37, CACNA2D4, |
| | | | CDHR1, CERKL, CNGA3, CNGB3, |
| | | | CNNM4, CNAT2, IFT81, KCNV2, |
| | | | PDE6C, PDE6H, POC1B, RAX2, |
| | | | RDH5, RPGRIP1, TTLL5, RetCG1, |
| | | | GUCY2E |
| Congenital Stationary Night | eye | | CABP4, CACNA1F, CACNA2D4, |
| Blindness | | | GNAT1, CPR179, GRK1, GRM6, |
| | | | LRIT3, NYX, PDE6B, RDH5, RHO, |
| | | | RLBP1, RPE65, SAG, SLC24A1, |
| | | | TRPM1, |
| Congenital Fructose Intolerance | Metabolism | | ALDOB |
| Cori's Disease (Glycogen Storage | Various- | | AGL |
| Disease Type III) | wherever | | |
| | glycogen | | |
| | accumulates, | | |
| | particularly | | |
| | liver, heart, | | |
| | skeletal muscle | | |
| Corneal clouding and dystrophy | eye | | APOA1, TGFBI, CSD2, CDGG1, |
| | | | CSD, BIGH3, CDG2, TACSTD2, |
| | | | TROP2, M1S1, VSX1, RINX, PPCD, |
| | | | PPD, KTCN, COL8A2, FECD, |
| | | | PPCD2, PIP5K3, CFD |
| Cornea plana congenital | | | KERA, CNA2 |
| Cri du chat Syndrome, also | | | Deletions involving only band 5p15.2 |
| known as 5p syndrome and cat | | | to the entire short arm of chromosome |
| cry syndrome | | | 5, e.g. CTNND2, TERT, |
| Cystic Fibrosis (CF) | Lungs and | Pancreas, liver, | CTFR, ABCC7, CF, MRP7, SCNN1A, |
| | respiratory | digestive | those described in WO2015157070 |
| | system | system, | |
| | | reproductive | |
| | | system, | |
| | | exocrine, glands, | |
| Diabetic nephropathy | kidney | | Gremlin, 12/15-lipoxygenase, TIM44, |
| Dent Disease (Types 1 and 2) | Kidney | | Type 1: CLCN5, Type 2: ORCL |
| Dentatorubro-Pallidoluysian | CNS, brain, | | Atrophin-1 and Atn1 |
| Atrophy (DRPLA) (aka Haw | muscle | | |
| River and Naito-Oyanagi | | | |
| Disease) | | | |
| Down Syndrome | various | | Chromosome 21 trisomy |
| Drug Addiction | Brain | | Prkce; Drd2; Drd4; ABAT; |
| | | | GRIA2; Grm5; Grin1; Htr1b; Grin2a; |
| | | | Drd3; Pdyn; Gria1 |
| Duane syndrome (Types 1, 2, and | eye | | CHN1, indels on chromosomes 4 and 8 |
| 3, including subgroups A, B and | | | |
| C). Other names for this | | | |
| condition include: Duane's | | | |
| Retraction Syndrome (or DR | | | |
| syndrome), Eye Retraction | | | |
| Syndrome, Retraction Syndrome, | | | |
| Congenital retraction syndrome | | | |
| and Stilling-Turk-Duane | | | |
| Syndrome | | | |
| Duchenne muscular dystrophy | muscle | Cardiovascular, | DMD, BMD, dystrophin gene, intron |
| (DMD) | | respiratory | flanking exon 51 of DMD gene, exon |
| | | | 51 mutations in DMD gene, see also |
| | | | WO2013163628 and US Pat. Pub. |
| | | | 20130145487 |
| Edward's Syndrome | | | Complete or partial trisomy of |
| (Trisomy 18) | | | chromosome 18 |
| Ehlers-Danlos Syndrome (Types | Various | | COL5A1, COL5A2, COL1A1, |
| I-VI) | depending on | | COL3A1, TNXB, PLOD1, COL1A2, |
| | type: including | | FKBP14 and ADAMTS2 |
| | musculoskeletal, | | |
| | eye, vasculature, | | |
| | immune, and | | |
| | skin | | |
| Emery-Dreifuss muscular | muscle | | LMNA, LMN1, EMD2, FPLD, |
| dystrophy | | | CMD1A, HGPS, LGMD1B, LMNA, |
| | | | LMN1, EMD2, FPLD, CMD1A |
| Enhanced S-Cone Syndrome | eye | | NR2E3, NRL |
| Fabry's Disease | Various- | | GLA |
| | including skin, | | |
| | eyes, and | | |
| | gastrointestinal | | |
| | system, kidney, | | |
| | heart, brain, | | |
| | nervous system | | |
| Facioscapulohumeral muscular | muscles | | FSHMD1A, FSHD1A, FRG1, |
| dystrophy | | | |
| Factor H and Factor H-like 1 | blood | | HF1, CFH, HUS |
| Factor V Leiden thrombophilia | blood | | Factor V (F5) |
| and Factor V deficiency | | | |
| Factor V and Factor VII | blood | | MCFD2 |
| deficiency | | | |
| Factor VII deficiency | blood | | F7 |
| Factor X deficiency | blood | | F10 |
| Factor XI deficiency | blood | | F11 |
| Factor XII deficiency | blood | | F12, HAF |
| Factor XIIIA deficiency | blood | | F13A1, F13A |
| Factor XIIIB deficiency | blood | | F13B |
| Familial Hypercholestereolemia | Cardiovascular | | APOB, LDLR, PCSK9 |
| | system | | |
| Familial Mediterranean Fever | Various- | Heart, kidney, | MEFV |
| (FMF) also called recurrent | organs/tissues | brain/CNS, | |
| polyserositis or familial | with serous or | reproductive | |
| paroxysmal polyserositis | synovial | organs | |
| | membranes, | | |
| | skin, joints | | |
| Fanconi Anemia | Various-blood | | FANCA, FACA, FA1, FA, FAA, |
| | (anemia), | | FAAP95, FAAP90, FLJ34064, |
| | immune system, | | FANCC, FANCG, RAD51, BRCA1, |
| | cognitive, | | BRCA2, BRIP1, BACH1, FANCJ, |
| | kidneys, eyes, | | FANCB, FANCD1, FANCD2, |
| | musculoskeletal | | FANCD, FAD, FANCE, FACE, |
| | | | FANCF, FANCI, ERCC4, FANCL, |
| | | | FANCM, PALB2, RAD51C, SLX4, |
| | | | UBE2T, FANCB, XRCC9, PHF9, |
| | | | KIAA1596 |
| Fanconi Syndrome Types I | kidneys | | FRTS1, GATM |
| (Childhood onset) and II (Adult | | | |
| Onset) | | | |
| Fragile X syndrome and related | brain | | FMR1, FMR2; FXR1; FXR2; |
| disorders | | | mGLUR5 |
| Fragile XE Mental Retardation | Brain, nervous | | FMR1 |
| (aka Martin Bell syndrome) | system | | |
| Friedreich Ataxia (FRDA) | Brain, nervous | heart | FXN/X25 |
| | system | | |
| Fuchs endothelial corneal | Eye | | TCF4; COL8A2 |
| dystrophy | | | |
| Galactosemia | Carbohydrate | Various-where | GALT, GALK1, and GALE |
| | metabolism | galactose | |
| | disorder | accumulates- | |
| | | liver, brain, eyes | |
| Gastrointestinal Epithelial | | | CISH |
| Cancer, GI cancer | | | |
| Gaucher Disease (Types 1, 2, and | Fat metabolism | Various-liver, | GBA |
| 3, as well as other unusual forms | disorder | spleen, blood, | |
| that may not fit into these types) | | CNS, skeletal | |
| | | system | |
| Griscelli syndrome | | | |
| Glaucoma | eye | | MYOC, TIGR, GLC1A, JOAG, |
| | | | GPOA, OPTN, GLC1E, FIP2, HYPL, |
| | | | NRP, CYP1B1, GLC3A, OPA1, NTG, |
| | | | NPG, CYP1B1, GLC3A, those |
| | | | described in WO2015153780 |
| Glomerulo sclerosis | kidney | | CC chemokine ligand 2 |
| Glycogen Storage Diseases | Metabolism | | SLC2A2, GLUT2, G6PC, G6PT, |
| Types I-VI-See also Cori’s | Diseases | | G6PT1, GAA, LAMP2, LAMPB, |
| Disease, Pompe’s Disease, | | | AGL, GDE, GBE1, GYS2, PYGL, |
| McArdle’s disease, Hers Disease, | | | PFKM, see also Cori’s Disease, |
| and Von Gierke’s disease | | | Pompe’s Disease, McArdle’s disease, |
| | | | Hers Disease, and Von Gierke’s |
| | | | disease |
| RBC Glycolytic enzyme | blood | | any mutations in a gene for an enzyme |
| deficiency | | | in the glycolysis pathway including |
| | | | mutations in genes for hexokinases I |
| | | | and II, glucokinase, phosphoglucose |
| | | | isomerase, phosphofructokinase, |
| | | | aldolase Bm triosephosphate |
| | | | isomerease, glyceraldehydee-3- |
| | | | phosphate dehydrogenase, |
| | | | phosphoglycerokinase, |
| | | | phosphoglycerate mutase, enolase I, |
| | | | pyruvate kinase |
| Hartnup's disease | Malabsorption | Various-brain, | SLC6A19 |
| | disease | gastrointestinal, | |
| | | skin, | |
| Hearing Loss | ear | | NOX3, Hes5, BDNF, |
| Hemochromatosis (HH) | Iron absorption | Various- | HFE and H63D |
| | regulation | wherever iron | |
| | disease | accumulates, | |
| | | liver, heart, | |
| | | pancreas, joints, | |
| | | pituitary gland | |
| Hemophagocytic | blood | | PRF1, HPLH2, UNC13D, MUNC13- |
| lymphohistiocytosis disorders | | | 4, HPLH3, HLH3, FHL3 |
| Hemorrhagic disorders | blood | | PI, ATT, F5 |
| Hers disease (Glycogen storage | liver | muscle | PYGL |
| disease Type VI) | | | |
| Hereditary angioedema (HAE) | | | kalikrein B1 |
| Hereditary Hemorrhagic | Skin and | | ACVRL1, ENG and SMAD4 |
| Telangiectasia (Osler-Weber- | mucous | | |
| Rendu Syndrome) | membranes | | |
| Hereditary Spherocytosis | blood | | NK1, EPB42, SLC4A1, SPTA1, and |
| | | | SPTB |
| Hereditary Persistence of Fetal | blood | | HBG1, HBG2, BCL11A, promoter |
| Hemoglobin | | | region of HBG 1 and/or 2 (in the |
| | | | CCAATbox) |
| Hemophilia (hemophilia A | blood | | A: FVIII, F8C, HEMA |
| (Classic) a B (aka Christmas | | | B: FVIX, HEMB |
| disease) and C) | | | C: F9, F11 |
| Hepatic adenoma | liver | | TCF1, HNF1A, MODY3 |
| Hepatic failure, early onset, and | liver | | SCOD1, SCO1 |
| neurologic disorder | | | |
| Hepatic lipase deficiency | liver | | LIPC |
| Hepatoblastoma, cancer and | liver | | CTNNB1, PDGFRL, PDGRL, PRLTS, |
| carcinomas | | | AXIN1, AXIN, CTNNB1, TP53, P53, |
| | | | LFS1, IGF2R, MPRI, MET, CASP8, |
| | | | MCH5 |
| Hermansky-Pudlak syndrome | Skin, eyes, | | HPS1, HPS3, HPS4, HPS5, HPS6, |
| | blood, lung, | | HPS7, DTNBP1, BLOC1, BLOC1S2, |
| | kidneys, | | BLOC3 |
| | intestine | | |
| HIV susceptibility or infection | Immune system | | IL10, CSIF, CMKBR2, CCR2, |
| | | | CMKBR5, CCCKR5 (CCR5), those in |
| | | | WO2015148670A1 |
| Holoprosencephaly (HPE) | brain | | ACVRL1, ENG, SMAD4 |
| (Alobar, Semilobar, and Lobar) | | | |
| Homocystinuria | Metabolic | Various- | CBS, MTHFR, MTR, MTRR, and |
| | disease | connective | MMADHC |
| | | tissue, muscles, | |
| | | CNS, | |
| | | cardiovascular | |
| | | system | |
| HPV | | | HPV16 and HPV18 E6/E7 |
| HSV1, HSV2, and related | eye | | HSV1 genes (immediate early and late |
| keratitis | | | HSV-1 genes (UL1, 1.5, 5, 6, 8, 9, 12, |
| | | | 15, 16, 18, 19, 22, 23, 26, 26.5, 27, 28, |
| | | | 29, 30, 31, 32, 33, 34, 35, 36, 37, 38, |
| | | | 42, 48, 49.5, 50, 52, 54, S6, RL2, RS1, |
| | | | those described in WO2015153789, |
| | | | WO2015153791 |
| Hunter's Syndrome (aka | Lysosomal | Various-liver, | IDS |
| Mucopolysaccharidosis type II) | storage disease | spleen, eye, | |
| | | joint, heart, | |
| | | brain, skeletal | |
| Huntington's disease (HD) and | Brain, nervous | | HD, HTT, IT15, PRNP, PRIP, JPH3, |
| HD-like disorders | system | | JP3, HDL2, TBP, SCA17, PRKCE; |
| | | | IGF1; EP300; RCOR1; PRKCZ; |
| | | | HDAC4; and TGM2, and those |
| | | | described in WO2013130824, |
| | | | WO2015089354 |
| Hurler's Syndrome (aka | Lysosomal | Various-liver, | IDUA, α-L-iduronidase |
| mucopolysaccharidosis type IH, | storage disease | spleen, eye, | |
| MPS IH) | | joint, heart, | |
| | | brain, skeletal | |
| Hurler-Scheie syndrome (aka | Lysosomal | Various-liver, | IDUA, α-L-iduronidase |
| mucopolysaccharidosis type I | storage disease | spleen, eye, | |
| H-S, MPS I H-S) | | joint, heart, | |
| | | brain, skeletal | |
| hyaluronidase deficiency (aka | Soft and | | HYAL1 |
| MPS IX) | connective | | |
| | tissues | | |
| Hyper IgM syndrome | Immune system | | CD40L |
| Hyper-tension caused renal | kidney | | Mineral corticoid receptor |
| damage | | | |
| Immunodeficiencies | Immune System | | CD3E, CD3G, AICDA, AID, HIGM2, |
| | | | TNFRSF5, CD40, UNG, DGU, |
| | | | HIGM4, TNFSF5, CD40LG, HIGM1, |
| | | | IGM, FOXP3, IPEX, AIID, XPID, |
| | | | PIDX, TNFRSF14B, TACI |
| Inborn errors of metabolism: | Metabolism | Various organs | See also: Carbohydrate metabolism |
| including urea cycle disorders, | diseases, liver | and cells | disorders (e.g. galactosemia), Amino |
| organic acidemias), fatty acid | | | acid Metabolism disorders (e.g. |
| oxidation defects, amino | | | phenylketonuria), Fatty acid |
| acidopathies, carbohydrate | | | metabolism (e.g. MCAD deficiency), |
| disorders, mitochondrial | | | Urea Cycle disorders (e.g. |
| disorders | | | Citrullinemia), Organic acidemias (e.g. |
| | | | Maple Syrup Urine disease), |
| | | | Mitochondrial disorders (e.g. |
| | | | MELAS), peroxisomal disorders (e.g. |
| | | | Zellweger syndrome) |
| Inflammation | Various | | IL-10; IL-1 (IL-1a; IL-1b); IL-13; IL- |
| | | | 17 (IL-17a (CTLA8); IL- |
| | | | 17b; IL-17c; IL-17d; IL-17f); II-23; |
| | | | Cx3cr1; ptpn22; TNFa; |
| | | | NOD2/CARD15 for IBD; IL-6; IL-12 |
| | | | (IL-12a; IL-12b); |
| | | | CTLA4; Cx3cl1 |
| Inflammatory Bowel Diseases | Gastrointestinal | Joints, skin | NOD2, IRGM, LRRK2, ATG5, |
| (e.g. Ulcerative Colitis and | | | ATG16L1, IRGM, GATM, ECM1, |
| Chron's Disease) | | | CDH1, LAMB1, HNF4A, GNA12, |
| | | | IL10, CARD9/15. CCR6, IL2RA, |
| | | | MST1, TNFSF15, REL, STAT3, |
| | | | IL23R, IL12B, FUT2 |
| Interstitial renal fibrosis | kidney | | TGF-β type II receptor |
| Job's Syndrome (aka Hyper IgE | Immune System | | STAT3, DOCK8 |
| Syndrome) | | | |
| Juvenile Retinoschisis | eye | | RS1, XLRS1 |
| Kabuki Syndrome 1 | | | MLL4, KMT2D |
| Kennedy Disease (aka | Muscles, brain, | | SBMA/SMAX1/AR |
| Spinobulbar Muscular Atrophy) | nervous system | | |
| Klinefelter syndrome | Various- | | Extra X chromosome in males |
| | particularly | | |
| | those involved | | |
| | in development | | |
| | of male | | |
| | characteristics | | |
| Lafora Disease | Brain, CNS | | EMP2A and EMP2B |
| Leber Congenital Amaurosis | eye | | CRB1, RP12, CORD2, CRD, CRX, |
| | | | IMPDH1, OTX2, AIPL1, CABP4, |
| | | | CCT2, CEP290, CLUAP1, CRB1, |
| | | | CRX, DTHD1, GDF6, GUCY2D, |
| | | | IFT140, IQCB1, KCNJ13, LCA5, |
| | | | LRAT, NMNAT1, PRPH2, RD3, |
| | | | RDH12, RPE65, RP20, RPGRIP1, |
| | | | SPATA7, TULP1, LCA1, LCA4, |
| | | | GUC2D, CORD6, LCA3, |
| Lesch-Nyhan Syndrome | Metabolism | Various-joints, | HPRT1 |
| | disease | cognitive, brain, | |
| | | nervous system | |
| Leukocyte deficiencies and | blood | | ITGB2, CD18, LCAMB, LAD, |
| disorders | | | EIF2B1, EIF2BA, EIF2B2, EIF2B3, |
| | | | EIF2B5, LVWM, CACH, CLE, |
| | | | EIF2B4 |
| Leukemia | Blood | | TAL1, TCL5, SCL, TAL2, FLT3, |
| | | | NBS1, NBS, ZNFN1A1, IK1, LYF1, |
| | | | HOXD4, HOX4B, BCR, CML, PHL, |
| | | | ALL, ARNT, KRAS2, RASK2, |
| | | | GMPS, AF10, ARHGEF12, LARG, |
| | | | KIAA0382, CALM, CLTH, CEBPA, |
| | | | CEBP, CHIC2, BTL, FLT3, KIT, |
| | | | PBT, LPP, NPM1, NUP214, D9S46E, |
| | | | CAN, CAIN, RUNX1, CBFA2, |
| | | | AML1, WHSC1L1, NSD3, FLT3, |
| | | | AFIQ, NPM1, NUMA1, ZNF145, |
| | | | PLZF, PML, MYL, STAT5B, AF10, |
| | | | CALM, CLTH, ARL11, ARLTS1, |
| | | | P2RX7, P2X7, BCR, CML, PHL, |
| | | | ALL, GRAF, NF1, VRNF, WSS, |
| | | | NFNS, PTPN11, PTP2C, SHP2, NS1, |
| | | | BCL2, CCND1, PRAD1, BCL1, |
| | | | TCRA, GATA1, GF1, ERYF1, NFE1, |
| | | | ABL1, NQO1, DIA4, NMOR1, |
| | | | NUP214, D9S46E, CAN, CAIN |
| Limb-girdle muscular dystrophy | muscle | | LGMD |
| diseases | | | |
| Lowe syndrome | brain, eyes, | | OCRL |
| | kidneys | | |
| Lupus glomerulo-nephritis | kidney | | MAPKI |
| Machado- | Brain, CNS, | | ATX3 |
| Joseph’s Disease (also known as | muscle | | |
| Spinocerebellar ataxia Type 3) | | | |
| Macular degeneration | eye | | ABC4, CBC1, CHM1, APOE, |
| | | | C1QTNF5, C2, C3, CCL2, CCR2, |
| | | | CD36, CFB, CFH, CFHR1, CFHR3, |
| | | | CNGB3, CP, CRP, CST3, CTSD, |
| | | | CX3CR1, ELOVL4, ERCC6, FBLN5, |
| | | | FBLN6, FSCN2, HMCN1, HTRA1, |
| | | | IL6, IL8, PLEKHA1, PROM1, |
| | | | PRPH2, RPGR, SERPING1, TCOF1, |
| | | | TIMP3, TLR3 |
| Macular Dystrophy | eye | | BEST1, C1QTNF5, CTNNA1, |
| | | | EFEMP1, ELOVL4, FSCN2, |
| | | | GUCA1B, HMCN1, IMPG1, OTX2, |
| | | | PRDM13, PROM1, PRPH2, RP1L1, |
| | | | TIMP3, ABCA4, CFH, DRAM2, |
| | | | IMG1, MFSD8, ADMD, STGD2, |
| | | | STGD3, RDS, RP7, PRPH, AVMD, |
| | | | AOFMD, VMD2 |
| Malattia Leventinesse | eye | | EFEMP1, FBLN3 |
| Maple Syrup Urine Disease | Metabolism | | BCKDHA, BCKDHB, and DBT |
| | disease | | |
| Marfan syndrome | Connective | Musculoskeletal | FBN1 |
| | tissue | | |
| Maroteaux-Lamy Syndrome (aka | Musculoskeletal | Liver, spleen | ARSB |
| MPS VI) | system, nervous | | |
| | system | | |
| McArdle's Disease (Glycogen | Glycogen | muscle | PYGM |
| Storage Disease Type V) | storage disease | | |
| Medullary cystic kidney disease | kidney | | UMOD, HNFJ, FJHN, MCKD2, |
| | | | ADMCKD2 |
| Metachromatic leukodystrophy | Lysosomal | Nervous system | ARSA |
| | storage disease | | |
| Methylmalonic acidemia (MMA) | Metabolism | | MMAA, MMAB, MUT, MMACHC, |
| | disease | | MMADHC, LMBRD1 |
| Morquio Syndrome (aka MPS IV | Connective | heart | GALNS |
| A andB) | tissue, skin, | | |
| | bone, eyes | | |
| Mucopolysaccharidosis diseases | Lysosomal | | See also Hurler/Scheie syndrome, |
| (Types I H/S, I H, II, III A B and | storage disease- | | Hurler disease, Sanfillipo syndrome, |
| C, I S, IVA and B, IX, VII, and | affects various | | Scheie syndrome, Morquio syndrome, |
| VI) | organs/tissues | | hyaluronidase deficiency, Sly |
| | | | syndrome, and Maroteaux-Lamy |
| | | | syndrome |
| Muscular Atrophy | muscle | | VAPB, VAPC, ALS8, SMN1, SMA1, |
| | | | SMA2, SMA3, SMA4, BSCL2, |
| | | | SPG17, GARS, SMAD1, CMT2D, |
| | | | HEXB, IGHMBP2, SMUBP2, |
| | | | CATF1, SMARD1 |
| Muscular dystrophy | muscle | | FKRP, MDC1C, LGMD2I, LAMA2, |
| | | | LAMM, LARGE, KIAA0609, |
| | | | MDC1D, FCMD, TTID, MYOT, |
| | | | CAPN3, CANP3, DYSF, LGMD2B, |
| | | | SGCG, LGMD2C, DMDA1, SCG3, |
| | | | SGCA, ADL, DAG2, LGMD2D, |
| | | | DMDA2, SGCB, LGMD2E, SGCD, |
| | | | SGD, LGMD2F, CMD1L, TCAP, |
| | | | LGMD2G, CMD1N, TRIM32, HT2A, |
| | | | LGMD2H, FKRP, MDC1C, LGMD2I, |
| | | | TTN, CMD1G, TMD, LGMD2J, |
| | | | POMT1, CAV3, LGMD1C, SEPN1, |
| | | | SELN, RSMD1, PLEC1, PLTN, EBS1 |
| Myotonic dystrophy (Type 1 and | Muscles | Eyes, heart, | CNBP (Type 2) and DMPK (Type 1) |
| Type 2) | | endocrine | |
| Neoplasia | | | PTEN; ATM; ATR; EGFR; ERBB2; |
| | | | ERBB3; ERBB4; |
| | | | Notch1; Notch2; Notch3; Notch4; |
| | | | AKT; AKT2; AKT3; HIF; |
| | | | HIF1a; HIF3a; Met; HRG; Bcl2; |
| | | | PPAR alpha; PPAR |
| | | | gamma; WT1 (Wilms Tumor); FGF |
| | | | Receptor Family |
| | | | members (5 members: 1, 2, 3, 4, 5); |
| | | | CDKN2a; APC; RB |
| | | | (retinoblastoma); MEN1; VHL; |
| | | | BRCA1; BRCA2; AR |
| | | | (Androgen Receptor); TSG101; IGF; |
| | | | IGF Receptor; Igf1 (4 |
| | | | variants); Igf2 (3 variants); Igf 1 |
| | | | Receptor; Igf 2 Receptor; |
| | | | Bax; Bcl2; caspases family (9 |
| | | | members: |
| | | | 1, 2, 3, 4, 6, 7, 8, 9, 12); Kras; Apc |
| Neurofibromatosis (NF) (NF1, | brain, spinal | | NF1, NF2 |
| formerly Recklinghausen's NF, | cord, nerves, | | |
| andNF2) | and skin | | |
| Niemann-Pick Lipidosis (Types | Lysosomal | Various-where | Types A and B: SMPD1; Type C: |
| A, B, and C) | Storage Disease | sphingomyelin | NPC1 orNPC2 |
| | | accumulates, | |
| | | particularly | |
| | | spleen, liver, | |
| | | blood, CNS | |
| Noonan Syndrome | Various- | | PTPN11, SOS1, RAF1 and KRAS |
| | musculoskeletal, | | |
| | heart, eyes, | | |
| | reproductive | | |
| | organs, blood | | |
| Norrie Disease or X-linked | eye | | NDP |
| Familial Exudative | | | |
| Vitreoretinopathy | | | |
| North Carolina Macular | eye | | MCDRI |
| Dystrophy | | | |
| Osteogenesis imperfecta (OI) | bones, | | COL1A1, COL1A2, CRTAP, P3H |
| (Types I, II, III, IV, V, VI, VII) | musculoskeletal | | |
| Osteopetrosis | bones | | LRP5, BMND1, LRP7, LR3, OPPG, |
| | | | VBCH2, CLCN7, CLC7, OPTA2, |
| | | | OSTM1, GL, TCIRG1, TIRC7, |
| | | | OC116, OPTB1 |
| Patau's Syndrome | Brain, heart, | | Additional copy of chromosome 13 |
| (Trisomy 13) | skeletal system | | |
| Parkinson's disease (PD) | Brain, nervous | | SNCA (PARK1), UCHL1 (PARK 5), |
| | system | | and LRRK2 (PARK8), (PARK3), |
| | | | PARK2, PARK4, PARK7 (PARK7), |
| | | | PINK1 (PARK6); x-Synuclein, DJ-1, |
| | | | Parkin, NR4A2, NURR1, NOT, |
| | | | TINUR, SNCAIP, TBP, SCA17, |
| | | | NCAP, PRKN, PDJ, DBH, NDUFV2 |
| Pattern Dystrophy of the RPE | eye | | RDS/peripherin |
| Phenylketonuria (PKU) | Metabolism | Various due to | PAH, PKU1, QDPR, DHPR, PTS |
| | disorder | build-up of | |
| | | phenylalanine, | |
| | | phenyl ketones | |
| | | in tissues and | |
| | | CNS | |
| Polycystic kidney and hepatic | Kidney, liver | | FCYT, PKHD1, ARPKD, PKD1, |
| disease | | | PKD2, PKD4, PKDTS, PRKCSH, |
| | | | G19P1, PCLD, SEC63 |
| Pompe's Disease | Glycogen | Various-heart, | GAA |
| | storage disease | liver, spleen | |
| Porphyria (actually refers to a | Various- | | ALAD, ALAS2, CPOX, FECH, |
| group of different diseases all | wherever heme | | HMBS, PPOX, UROD, or UROS |
| having a specific heme | precursors | | |
| production process abnormality) | accumulate | | |
| posterior polymorphous corneal | eyes | | TCF4; COL8A2 |
| dystrophy | | | |
| Primary Hyperoxaluria (e.g. type | Various-eyes, | | LDHA (lactate dehydrogenase A) and |
| 1) | heart, kidneys, | | hydroxyacid oxidase 1 (HAO1) |
| | skeletal system | | |
| Primary Open Angle Glaucoma | eyes | | MYOC |
| (POAG) | | | |
| Primary sclerosing cholangitis | Liver, | | TCF4; COL8A2 |
| | gallbladder | | |
| Progeria (also called Hutchinson- | All | | LMNA |
| Gilford progeria syndrome) | | | |
| Prader-Willi Syndrome | Musculoskeletal | | Deletion of region of short arm of |
| | system, brain, | | chromosome 15, including UBE3 A |
| | reproductive | | |
| | and endocrine | | |
| | system | | |
| Prostate Cancer | prostate | | HOXB13, MSMB, GPRC6A, TP53 |
| Pyruvate Dehydrogenase | Brain, nervous | | PDHA1 |
| Deficiency | system | | |
| Kidney/Renal carcinoma | kidney | | RLIP76, VEGF |
| Rett Syndrome | Brain | | MECP2, RTT, PPMX, MRX16, |
| | | | MRX79, CDKL5, STK9, MECP2, |
| | | | RTT, PPMX, MRX16, MRX79, x- |
| | | | Synuclein, DJ-1 |
| Retinitis pigmentosa (RP) | eye | | ADIPOR1, ABCA4, AGBL5, |
| | | | ARHGEF18, ARL2BP, ARL3, ARL6, |
| | | | BEST1, BBS1, BBS2, C2ORF71, |
| | | | C8ORF37, CA4, CERKL, CLRN1, |
| | | | CNGA1, CMGB1, CRB1, CRX, |
| | | | CYP4V2, DHDDS, DHX38, EMC1, |
| | | | EYS, FAM161A, FSCN2, GPR125, |
| | | | GUCA1B, HK1, HPRPF3, HGSNAT, |
| | | | IDH3B, IMPDH1, IMPG2, IFT140, |
| | | | IFT172, KLHL7, KIAA1549, KIZ, |
| | | | LRAT, MAK, MERTK, MVK, NEK2, |
| | | | NUROD1, NR2E3, NRL, OFD1, |
| | | | PDE6A, PDE6B, PDE6G, POMGNT1, |
| | | | PRCD, PROM1, PRPF3, PRPF4, |
| | | | PRPF6, PRPF8, PRPF31, PRPH2, |
| | | | RPB3, RDH12, REEP6, RP39, RGR, |
| | | | RHO, RLBP1, ROM1, RP1, RP1L1, |
| | | | RPY, RP2, RP9, RPE65, RPGR, |
| | | | SAMD11, SAG, SEMA4A, SLC7A14, |
| | | | SNRNP200, SPP2, SPATA7, TRNT1, |
| | | | TOPORS, TTC8, TULP1, USH2A, |
| | | | ZFN408, ZNF513, see also |
| | | | 20120204282 |
| Scheie syndrome (also known as | Various-liver, | | IDUA, α-L-iduronidase |
| mucopolysaccharidosis type I | spleen, eye, | | |
| S(MPS I-S)) | joint, heart, | | |
| | brain, skeletal | | |
| Schizophrenia | Brain | | Neuregulinl (Nrg1); Erb4 (receptor for |
| | | | Neuregulin); |
| | | | Complexini (Cplx1); Tph1 |
| | | | Tryptophan hydroxylase; Tph2 |
| | | | Tryptophan hydroxylase 2; Neurexin |
| | | | 1; GSK3; GSK3a; |
| | | | GSK3b; 5-HTT (Slc6a4); COMT; |
| | | | DRD (Drd1a); SLC6A3; DAOA; |
| | | | DTNBP1; Dao (Dao1); TCF4; |
| | | | COL8A2 |
| Secretase Related Disorders | Various | | APH-1 (alpha and beta); PSEN1; |
| | | | NCSTN; PEN-2; Nos1, Parp1, Nat1, |
| | | | Nat2, CTSB, APP, APH1B, PSEN2, |
| | | | PSENEN, BACE1, ITM2B, CTSD, |
| | | | NOTCH1, TNF, INS, DYT10, |
| | | | ADAM17, APOE, ACE, STN, TP53, |
| | | | IL6, NGFR, IL1B, ACHE, CTNNB1, |
| | | | IGF1, IFNG, NRG1, CASP3, MAPK1, |
| | | | CDH1, APBB1, HMGCR, CREB1, |
| | | | PTGS2, HES1, CAT, TGFB1, ENO2, |
| | | | ERBB4, TRAPPC10, MAOB, NGF, |
| | | | MMP12, JAG1, CD40LG, PPARG, |
| | | | FGF2, LRP1, NOTCH4, MAPK8, |
| | | | PREP, NOTCH3, PRNP, CTSG, EGF, |
| | | | REN, CD44, SELP, GHR, ADCYAP1, |
| | | | INSR, GFAP, MMP3, MAPK10, SP1, |
| | | | MYC, CTSE, PPARA, JUN, TIMP1, |
| | | | IL5, IL1A, MMP9, HTR4, HSPG2, |
| | | | KRAS, CYCS, SMG1, IL1R1, |
| | | | PROK1, MAPK3, NTRK1, IL13, |
| | | | MME, TKT, CXCR2, CHRM1, |
| | | | ATXN1, PAWR, NOTCJ2, M6PR, |
| | | | CYP46A1, CSNK1D, MAPK14, |
| | | | PRG2, PRKCA, L1 CAM, CD40, |
| | | | NR1I2, JAG2, CTNND1, CMA1, |
| | | | SORT1, DLK1, THEM4, JUP, CD46, |
| | | | CCL11, CAV3, RNASE3, HSPA8, |
| | | | CASP9, CYP3A4, CCR3, TFAP2A, |
| | | | SCP2, CDK4, JOF1A, TCF7L2, |
| | | | B3GALTL, MDM2, RELA, CASP7, |
| | | | IDE, FANP4, CASK, ADCYAP1R1, |
| | | | ATF4, PDGFA, C21ORF33, SCG5, |
| | | | RMF123, NKFB1, ERBB2, CAV1, |
| | | | MMP7, TGFA, RXRA, STX1A, |
| | | | PSMC4, P2RY2, TNFRSF21, DLG1, |
| | | | NUMBL, SPN, PLSCR1, UBQLN2, |
| | | | UBQLN1, PCSK7, SPON1, SILV, |
| | | | QPCT, HESS, GCC1 |
| Selective IgA Deficiency | Immune system | | Type 1: MSH5; Type 2: TNFRSF13B |
| Severe Combined | Immune system | | JAK3, JAKL, DCLRE1C, ARTEMIS, |
| Immunodeficiency (SCID) and | | | SCIDA, RAG1, RAG2, ADA, PTPRC, |
| SCID-X1, and ADA-SCID | | | CD45, LCA, IL7R, CD3D, T3D, |
| | | | IL2RG, SCIDX1, SCIDX, IMD4, |
| | | | those identified in US Pat. App. Pub. |
| | | | 20110225664, 20110091441, |
| | | | 20100229252, 20090271881 and |
| | | | 20090222937; |
| Sickle cell disease | blood | | HBB, BCL11A, BCL11Ae, cis- |
| | | | regulatory elements of the B-globin |
| | | | locus, HBG 1/2 promoter, HBG distal |
| | | | CCAAT box region between −92 and |
| | | | −130 of the HBG Transcription Start |
| | | | Site, those described in |
| | | | WO2015148863, WO 2013/126794, |
| | | | US Pat. Pub. 20110182867 |
| Sly Syndrome (aka MPS VII) | | | GUSB |
| Spinocerebellar Ataxias (SCA | | | ATXN1, ATXN2, ATX3 |
| types 1, 2, 3, 6, 7, 8, 12 and 17) | | | |
| Sorsby Fundus Dystrophy | eye | | TIMP3 |
| Stargardt disease | eye | | ABCR, ELOVL4, ABCA4, PROM1 |
| Tay-Sachs Disease | Lysosomal | Various-CNS, | HEX-A |
| | Storage disease | brain, eye | |
| Thalassemia (Alpha, Beta, Delta) | blood | | HBA1, HBA2 (Alpha), HBB (Beta), |
| | | | HBB and HBD (delta), LCRB, |
| | | | BCL11A, BCL11Ae, cis-regulatory |
| | | | elements of the B-globin locus, HBG |
| | | | 1/2 promoter, those described in |
| | | | WO2015148860, US Pat. Pub. |
| | | | 20110182867, 2015/148860 |
| Thymic Aplasia (DiGeorge | Immune system, | | deletion of 30 to 40 genes in the |
| Syndrome; 22q11.2 deletion | thymus | | middle of chromosome 22 at |
| syndrome) | | | a location known as 22q11.2, including |
| | | | TBX1, DGCR8 |
| Transthyretin amyloidosis | liver | | TTR (transthyretin) |
| (ATTR) | | | |
| trimethylaminuria | Metabolism | | FMO3 |
| | disease | | |
| Trinucleotide Repeat Disorders | Various | | HTT; SBMA/SMAX1/AR; |
| (generally) | | | FXN/X25 ATX3; |
| | | | ATXN1; ATXN2; |
| | | | DMPK; Atrophin-1 and Atn1 |
| | | | (DRPLA Dx); CBP (Creb-BP-global |
| | | | instability); VLDLR; Atxn7; Atxn10; |
| | | | FEN1, TNRC6A, PABPN1, JPH3, |
| | | | MED15, ATXN1, ATXN3, TBP, |
| | | | CACNA1A, ATXN80S, PPP2R2B, |
| | | | ATXN7, TNRC6B, TNRC6C, CELF3, |
| | | | MAB21L1, MSH2, TMEM185A, |
| | | | SIX5, CNPY3, RAXE, GNB2, RPL14, |
| | | | ATXN8, ISR, TTR, EP400, GIGYF2, |
| | | | OGG1, STC1, CNDP1, C10ORF2, |
| | | | MAML3, DKC1, PAXIP1, CASK, |
| | | | MAPT, SP1, POLG, AFF2, THBS1, |
| | | | TP53, ESR1, CGGBP1, ABT1, KLK3, |
| | | | PRNP, JUN, KCNN3, BAX, FRAXA, |
| | | | KBTBD10, MBNL1, RAD51, |
| | | | NCOA3, ERDA1, TSC1, COMP, |
| | | | GGLC, RRAD, MSH3, DRD2, CD44, |
| | | | CTCF, CCND1, CLSPN, MEF2A, |
| | | | PTPRU, GAPDH, TRIM22, WT1, |
| | | | AHR, GPX1, TPMT, NDP, ARX, |
| | | | TYR, EGR1, UNG, NUMBL, FABP2, |
| | | | EN2, CRYGC, SRP14, CRYGB, |
| | | | PDCD1, HOXA1, ATXN2L, PMS2, |
| | | | GLA, CBL, FTH1, IL12RB2, OTX2, |
| | | | HOXA5, POLG2, DLX2, AHRR, |
| | | | MANF, RMEM158, see also |
| | | | 20110016540 |
| Turner's Syndrome (XO) | Various- | | Monosomy X |
| | reproductive | | |
| | organs, and sex | | |
| | characteristics, | | |
| | vasculature | | |
| Tuberous Sclerosis | CNS, heart, | | TSC1, TSC2 |
| | kidneys | | |
| Usher syndrome (Types I, II, and | Ears, eyes | | ABHD12, CDH23, CIB2, CLRN1, |
| III) | | | DFNB31, GPR98, HARS, MYO7A, |
| | | | PCDH15, USH1C, USH1G, USH2A, |
| | | | USH11A, those described in |
| | | | WO2015134812A1 |
| Velocardiofacial syndrome (aka | Various- | | Many genes are deleted, COM, TBX1, |
| 22q11.2 deletion syndrome, | skeletal, heart, | | and other are associated with |
| DiGeorge syndrome, conotruncal | kidney, immune | | symptoms |
| anomaly face syndrome (CTAF), | system, brain | | |
| autosomal dominant Opitz G/BB | | | |
| syndrome or Cayler cardiofacial | | | |
| syndrome) | | | |
| Von Gierke's Disease (Glycogen | Glycogen | Various-liver, | G6PC and SLC37A4 |
| Storage Disease type I) | Storage disease | kidney | |
| Von Hippel-Lindau Syndrome | Various-cell | CNS, Kidney, | VHL |
| | growth | Eye, visceral | |
| | regulation | organs | |
| | disorder | | |
| Von Willebrand Disease (Types | blood | | VWF |
| I, II and III) | | | |
| Wilson Disease | Various- | Liver, brains, | ATP7B |
| | Copper Storage | eyes, other | |
| | Disease | tissues where | |
| | | copper builds up | |
| Wiskott-Aldrich Syndrome | Immune System | | WAS |
| Xeroderma Pigmentosum | Skin | Nervous system | POLH |
| XXX Syndrome | Endocrine, brain | | X chromosome trisomy |

In some embodiments, the compositions, systems, or components thereof can be used treat or prevent a disease in a subject by modifying one or more genes associated with one or more cellular functions, such as any one or more of those in Table 11. In some embodiments, the disease is a genetic disease or disorder. In some of embodiments, the composition, system, or component thereof can modify one or more genes or polynucleotides associated with one or more genetic diseases such as any set forth in Table 11.

TABLE 11
Exemplary Genes controlling Cellular Functions

| CELLULAR FUNCTION | GENES |
|---|---|
| PI3K/AKT Signaling | PRKCE; ITGAM; ITGA5; IRAK1; PRKAA2; EIF2AK2; PTEN; EIF4E; |
| | PRKCZ; GRK6; MAPK1; TSC1; PLK1; AKT2; IKBKB; PIK3CA; CDK8; |
| | CDKN1B; NFKB2; BCL2; PIK3CB; PPP2R1A; MAPK8; BCL2L1; MAPK3; |
| | TSC2; ITGA1; KRAS; EIF4EBP1; RELA; PRKCD; NOS3; PRKAA1; |
| | MAPK9; CDK2; PPP2CA; PIM1; ITGB7; YWHAZ; ILK; TP53; RAF1; |
| | IKBKG; RELB; DYRK1A; CDKN1A; ITGB1; MAP2K2; JAK1; AKT1; JAK2; |
| | PIK3R1; CHUK; PDPK1; PPP2R5C; CTNNB1; MAP2K1; NFKB1; PAK3; |
| | ITGB3; CCND1; GSK3A; FRAP1; SFN; ITGA2; TTK; CSNK1A1; BRAF; |
| | GSK3B; AKT3; FOXO1; SGK; HSP90AA1; RPS6KB1 |
| ERK/MAPK Signaling | PRKCE; ITGAM; ITGA5; HSPB1; IRAK1; PRKAA2; EIF2AK2; RAC1; |
| | RAP1A; TLN1; EIF4E; ELK1; GRK6; MAPK1; RAC2; PLK1; AKT2; |
| | PIK3CA; CDK8; CREB1; PRKCI; PTK2; FOS; RPS6KA4; PIK3CB; |
| | PPP2R1A; PIK3C3; MAPK8; MAPK3; ITGA1; ETS1; KRAS; MYCN; |
| | EIF4EBP1; PPARG; PRKCD; PRKAA1; MAPK9; SRC; CDK2; PPP2CA; |
| | PIM1; PIK3C2A; ITGB7; YWHAZ; PPP1CC; KSR1; PXN; RAF1; FYN; |
| | DYRK1A; ITGB1; MAP2K2; PAK4; PIK3R1; STAT3; PPP2R5C; MAP2K1; |
| | PAK3; ITGB3; ESR1; ITGA2; MYC; TTK; CSNK1A1; CRKL; BRAF; ATF4; |
| | PRKCA; SRF; STAT1; SGK |
| Glucocorticoid Receptor | RAC1; TAF4B; EP300; SMAD2; TRAF6; PCAF; ELK1; MAPK1; SMAD3; |
| Signaling | AKT2; IKBKB; NCOR2; UBE2I; PIK3CA; CREB1; FOS; HSPA5; NFKB2; |
| | BCL2; MAP3K14; STAT5B; PIK3CB; PIK3C3; MAPK8; BCL2L1; MAPK3; |
| | TSC22D3; MAPK10; NRIP1; KRAS; MAPK13; RELA; STAT5A; MAPK9; |
| | NOS2A; PBX1; NR3C1; PIK3C2A; CDKN1C; TRAF2; SERPINE1; NCOA3; |
| | MAPK14; TNF; RAF1; IKBKG; MAP3K7; CREBBP; CDKN1A; MAP2K2; |
| | JAK1; IL8; NCOA2; AKT1; JAK2; PIK3R1; CHUK; STAT3; MAP2K1; |
| | NFKB1; TGFBR1; ESR1; SMAD4; CEBPB; JUN; AR; AKT3; CCL2; MMP1; |
| | STAT1; IL6; HSP90AA1 |
| Axonal Guidance Signaling | PRKCE; ITGAM; ROCK1; ITGA5; CXCR4; ADAM12; IGF1; RAC1; RAP1A; |
| | EIF4E; PRKCZ; NRP1; NTRK2; ARHGEF7; SMO; ROCK2; MAPK1; PGF; |
| | RAC2; PTPN11; GNAS; AKT2; PIK3CA; ERBB2; PRKCI; PTK2; CFL1; |
| | GNAQ; PIK3CB; CXCL12; PIK3C3; WNT11; PRKD1; GNB2L1; ABL1; |
| | MAPK3; ITGA1; KRAS; RHOA; PRKCD; PIK3C2A; ITGB7; GLI2; PXN; |
| | VASP; RAF1; FYN; ITGB1; MAP2K2; PAK4; ADAM17; AKT1; PIK3R1; |
| | GLH; WNT5A; ADAM10; MAP2K1; PAK3; ITGB3; CDC42; VEGFA; |
| | ITGA2; EPHA8; CRKL; RND1; GSK3B; AKT3; PRKCA |
| Ephrin Receptor Signaling | PRKCE; ITGAM; ROCK1; ITGA5; CXCR4; IRAK1; PRKAA2; EIF2AK2; |
| Actin Cytoskeleton | RAC1; RAP1A; GRK6; ROCK2; MAPK1; PGF; RAC2; PTPN11; GNAS; |
| Signaling | PLK1; AKT2; DOK1; CDK8; CREB1; PTK2; CFL1; GNAQ; MAP3K14; |
| | CXCL12; MAPK8; GNB2L1; ABL1; MAPK3; ITGA1; KRAS; RHOA; |
| | PRKCD; PRKAA1; MAPK9; SRC; CDK2; PIM1; ITGB7; PXN; RAF1; FYN; |
| | DYRK1A; ITGB1; MAP2K2; PAK4; AKT1; JAK2; STAT3; ADAM10; |
| | MAP2K1; PAK3; ITGB3; CDC42; VEGFA; ITGA2; EPHA8; TTK; |
| | CSNK1A1; CRKL; BRAF; PTPN13; ATF4; AKT3; SGK |
| | ACTN4; PRKCE; ITGAM; ROCK1; ITGA5; IRAK1; PRKAA2; EIF2AK2; |
| | RAC1; INS; ARHGEF7; GRK6; ROCK2; MAPK1; RAC2; PLK1; AKT2; |
| | PIK3CA; CDK8; PTK2; CFL1; PIK3CB; MYH9; DIAPH1; PIK3C3; MAPK8; |
| | F2R; MAPK3; SLC9A1; ITGA1; KRAS; RHOA; PRKCD; PRKAA1; MAPK9; |
| | CDK2; PIM1; PIK3C2A; ITGB7; PPPICC; PXN; VIL2; RAF1; GSN; |
| | DYRK1A; ITGB1; MAP2K2; PAK4; PIP5K1A; PIK3R1; MAP2K1; PAK3; |
| | ITGB3; CDC42; APC; ITGA2; TTK; CSNK1A1; CRKL; BRAF; VAV3; SGK |
| Huntington’s Disease | PRKCE; IGF1; EP300; RCOR1; PRKCZ; HDAC4; TGM2; MAPK1; CAPNS1; |
| Signaling | AKT2; EGFR; NCOR2; SP1; CAPN2; PIK3CA; HDAC5; CREB1; PRKCI; |
| | HSPA5; REST; GNAQ; PIK3CB; PIK3C3; MAPK8; IGFIR; PRKD1; |
| | GNB2L1; BCL2L1; CAPN1; MAPK3; CASP8; HDAC2; HDAC7A; PRKCD; |
| | HDAC11; MAPK9; HDAC9; PIK3C2A; HDAC3; TP53; CASP9; CREBBP; |
| | AKT1; PIK3R1; PDPK1; CASP1; APAF1; FRAP1; CASP2; JUN; BAX; ATF4; |
| | AKT3; PRKCA; CLTC; SGK; HDAC6; CASP3 |
| Apoptosis Signaling | PRKCE; ROCK1; BID; IRAK1; PRKAA2; EIF2AK2; BAK1; BIRC4; GRK6; |
| | MAPK1; CAPNS1; PLK1; AKT2; IKBKB; CAPN2; CDK8; FAS; NFKB2; |
| | BCL2; MAP3K14; MAPK8; BCL2L1; CAPN1; MAPK3; CASP8; KRAS; |
| | RELA; PRKCD; PRKAA1; MAPK9; CDK2; PIM1; TP53; TNF; RAF1; |
| | IKBKG; RELB; CASP9; DYRK1A; MAP2K2; CHUK; APAF1; MAP2K1; |
| | NFKB1; PAK3; LMNA; CASP2; BIRC2; TTK; CSNK1A1; BRAF; BAX; |
| | PRKCA; SGK; CASP3; BIRC3; PARP1 |
| B Cell Receptor Signaling | RAC1; PTEN; LYN; ELK1; MAPK1; RAC2; PTPN11; AKT2; IKBKB; |
| | PIK3CA; CREB1; SYK; NFKB2; CAMK2A; MAP3K14; PIK3CB; PIK3C3; |
| | MAPK8; BCL2L1; ABL1; MAPK3; ETS1; KRAS; MAPK13; RELA; PTPN6; |
| | MAPK9; EGR1; PIK3C2A; BTK; MAPK14; RAF1; IKBKG; RELB; MAP3K7; |
| | MAP2K2; AKT1; PIK3R1; CHUK; MAP2K1; NFKB1; CDC42; GSK3A; |
| | FRAP1; BCL6; BCL10; JUN; GSK3B; ATF4; AKT3; VAV3; RPS6KB1 |
| Leukocyte Extravasation | ACTN4; CD44; PRKCE; ITGAM; ROCK1; CXCR4; CYBA; RAC1; RAP1A; |
| Signaling | PRKCZ; ROCK2; RAC2; PTPN11; MMP14; PIK3CA; PRKC1; PTK2; |
| | PIK3CB; CXCL12; PIK3C3; MAPK8; PRKD1; ABL1; MAPK10; CYBB; |
| | MAPK13; RHOA; PRKCD; MAPK9; SRC; PIK3C2A; BTK; MAPK14; |
| | NOX1; PXN; VIL2; VASP; ITGB1; MAP2K2; CTNND1; PIK3R1; CTNNB1; |
| | CLDN1; CDC42; F11R; ITK; CRKL; VAV3; CTTN; PRKCA; MMP1; MMP9 |
| Integrin Signaling | ACTN4; ITGAM; ROCK1; ITGA5; RAC1; PTEN; RAP1A; TLN1; ARHGEF7; |
| | MAPK1; RAC2; CAPNS1; AKT2; CAPN2; PIK3CA; PTK2; PIK3CB; |
| | PIK3C3; MAPK8; CAV1; CAPN1; ABL1; MAPK3; ITGA1; KRAS; RHOA; |
| | SRC; PIK3C2A; ITGB7; PPPICC; ILK; PXN; VASP; RAF1; FYN; ITGB1; |
| | MAP2K2; PAK4; AKT1; PIK3R1; TNK2; MAP2K1; PAK3; ITGB3; CDC42; |
| | RND3; ITGA2; CRKL; BRAF; GSK3B; AKT3 |
| Acute Phase Response | IRAK1; SOD2; MYD88; TRAF6; ELK1; MAPK1; PTPN11; AKT2; IKBKB; |
| Signaling | PIK3CA; FOS; NFKB2; MAP3K14; PIK3CB; MAPK8; RIPK1; MAPK3; |
| | IL6ST; KRAS; MAPK13; IL6R; RELA; SOCS1; MAPK9; FTL; NR3C1; |
| | TRAF2; SERPINE1; MAPK14; TNF; RAF1; PDK1; IKBKG; RELB; |
| | MAP3K7; MAP2K2; AKT1; JAK2; PIK3R1; CHUK; STAT3; MAP2K1; |
| | NFKB1; FRAPI; CEBPB; JUN; AKT3; IL1R1; IL6 |
| PTEN Signaling | ITGAM; ITGA5; RAC1; PTEN; PRKCZ; BCL2L11; MAPK1; RAC2; AKT2; |
| | EGFR; IKBKB; CBL; PIK3CA; CDKN1B; PTK2; NFKB2; BCL2; PIK3CB; |
| | BCL2L1; MAPK3; ITGA1; KRAS; ITGB7; ILK; PDGFRB; INSR; RAF1; |
| | IKBKG; CASP9; CDKN1A; ITGB1; MAP2K2; AKT1; PIK3R1; CHUK; |
| | PDGFRA; PDPKI; MAP2K1; NFKB1; ITGB3; CDC42; CCND1; GSK3A; |
| | ITGA2; GSK3B; AKT3; FOXO1; CASP3; RPS6KB1 |
| p53 Signaling | PTEN; EP300; BBC3; PCAF; FASN; BRCA1; GADD45A; BIRC5; AKT2; |
| Signaling | PIK3CA; CHEK1; TP53INP1; BCL2; PIK3CB; PIK3C3; MAPK8; THBS1; |
| Aryl Hydrocarbon Receptor | ATR; BCL2L1; E2F1; PMAIP1; CHEK2; TNFRSF10B; TP73; RB1; HDAC9; |
| | CDK2; PIK3C2A; MAPK14; TP53; LRDD; CDKN1A; HIPK2; AKT1; |
| | PIK3R1; RRM2B; APAFI; CTNNB1; SIRT1; CCND1; PRKDC; ATM; SFN; |
| | CDKN2A; JUN; SNAI2; GSK3B; BAX; AKT3 |
| | HSPB1; EP300; FASN; TGM2; RXRA; MAPK1; NQO1; NCOR2; SP1; |
| | ARNT; CDKN1B; FOS; CHEK1; SMARCA4; NFKB2; MAPK8; ALDH1A1; |
| | ATR; E2F1; MAPK3; NRIP1; CHEK2; RELA; TP73; GSTPI; RB1; SRC; |
| | CDK2; AHR; NFE2L2; NCOA3; TP53; TNF; CDKN1A; NCOA2; APAFI; |
| | NFKB1; CCND1; ATM; ESR1; CDKN2A; MYC; JUN; ESR2; BAX; IL6; |
| | CYP1B1; HSP90AA1 |
| Xenobiotic Metabolism | PRKCE; EP300; PRKCZ; RXRA; MAPK1; NQO1; NCOR2; PIK3CA; ARNT; |
| Signaling | PRKC1; NFKB2; CAMK2A; PIK3CB; PPP2R1A; PIK3C3; MAPK8; PRKD1; |
| | ALDH1A1; MAPK3; NRIP1; KRAS; MAPK13; PRKCD; GSTPI; MAPK9; |
| | NOS2A; ABCB1; AHR; PPP2CA; FTL; NFE2L2; PIK3C2A; PPARGC1A; |
| | MAPK14; TNF; RAF1; CREBBP; MAP2K2; PIK3R1; PPP2R5C; MAP2K1; |
| | NFKB1; KEAP1; PRKCA; EIF2AK3; IL6; CYP1B1; HSP90AA1 |
| SAPK/JNK Signaling | PRKCE; IRAK1; PRKAA2; EIF2AK2; RAC1; ELK1; GRK6; MAPK1; |
| | GADD45A; RAC2; PLK1; AKT2; PIK3CA; FADD; CDK8; PIK3CB; PIK3C3; |
| | MAPK8; RIPK1; GNB2L1; IRS1; MAPK3; MAPK10; DAXX; KRAS; |
| | PRKCD; PRKAA1; MAPK9; CDK2; PIM1; PIK3C2A; TRAF2; TP53; LCK; |
| | MAP3K7; DYRK1A; MAP2K2; PIK3R1; MAP2K1; PAK3; CDC42; JUN; |
| | TTK; CSNK1A1; CRKL; BRAF; SGK |
| PPAr/RXR Signaling | PRKAA2; EP300; INS; SMAD2; TRAF6; PPARA; FASN; RXRA; MAPK1; |
| | SMAD3; GNAS; IKBKB; NCOR2; ABCA1; GNAQ; NFKB2; MAP3K14; |
| | STAT5B; MAPK8; IRS1; MAPK3; KRAS; RELA; PRKAA1; PPARGC1A; |
| | NCOA3; MAPK14; INSR; RAF1; IKBKG; RELB; MAP3K7; CREBBP; |
| | MAP2K2; JAK2; CHUK; MAP2K1; NFKB1; TGFBR1; SMAD4; JUN; IL1R1; |
| | PRKCA; IL6; HSP90AA1; ADIPOQ |
| NF-KB Signaling | IRAK1; EIF2AK2; EP300; INS; MYD88; PRKCZ; TRAF6; TBK1; AKT2; |
| | EGFR; IKBKB; PIK3CA; BTRC; NFKB2; MAP3K14; PIK3CB; PIK3C3; |
| | MAPK8; RIPK1; HDAC2; KRAS; RELA; PIK3C2A; TRAF2; TLR4; |
| | PDGFRB; TNF; INSR; LCK; IKBKG; RELB; MAP3K7; CREBBP; AKT1; |
| | PIK3R1; CHUK; PDGFRA; NFKB1; TLR2; BCL10; GSK3B; AKT3; |
| | TNFAIP3; IL1R1 |
| Neuregulin Signaling | ERBB4; PRKCE; ITGAM; ITGA5; PTEN; PRKCZ; ELK1; MAPK1; PTPN11; |
| Signaling | AKT2; EGFR; ERBB2; PRKC1; CDKN1B; STAT5B; PRKD1; MAPK3; |
| | ITGA1; KRAS; PRKCD; STAT5A; SRC; ITGB7; RAF1; ITGB1; MAP2K2; |
| | ADAM17; AKT1; PIK3R1; PDPKI; MAP2K1; ITGB3; EREG; FRAPI; |
| | PSEN1; ITGA2; MYC; NRG1; CRKL; AKT3; PRKCA; HSP90AA1; |
| | RPS6KB1 |
| Wnt & Beta catenin | CD44; EP300; LRP6; DVL3; CSNKIE; GJA1; SMO; AKT2; PIN1; CDH1; |
| | BTRC; GNAQ; MARK2; PPP2R1A; WNT11; SRC; DKKI; PPP2CA; SOX6; |
| | SFRP2; ILK; LEF1; SOX9; TP53; MAP3K7; CREBBP; TCF7L2; AKT1; |
| | PPP2R5C; WNT5A; LRP5; CTNNB1; TGFBR1; CCND1; GSK3A; DVL1; |
| | APC; CDKN2A; MYC; CSNK1A1; GSK3B; AKT3; SOX2 |
| Insulin Receptor Signaling | PTEN; INS; EIF4E; PTPN1; PRKCZ; MAPK1; TSC1; PTPN11; AKT2; CBL; |
| | PIK3CA; PRKC1; PIK3CB; PIK3C3; MAPK8; IRS1; MAPK3; TSC2; KRAS; |
| | EIF4EBP1; SLC2A4; PIK3C2A; PPPICC; INSR; RAF1; FYN; MAP2K2; |
| | JAK1; AKT1; JAK2; PIK3R1; PDPKI; MAP2K1; GSK3A; FRAP1; CRKL; |
| | GSK3B; AKT3; FOXO1; SGK; RPS6KB1 |
| IL-6 Signaling | HSPB1; TRAF6; MAPKAPK2; ELK1; MAPK1; PTPN11; IKBKB; FOS; |
| | NFKB2; MAP3K14; MAPK8; MAPK3; MAPK10; IL6ST; KRAS; MAPK13; |
| | IL6R; RELA; SOCS1; MAPK9; ABCB1; TRAF2; MAPK14; TNF; RAF1; |
| | IKBKG; RELB; MAP3K7; MAP2K2; IL8; JAK2; CHUK; STAT3; MAP2K1; |
| | NFKB1; CEBPB; JUN; IL1R1; SRF; IL6 |
| Hepatic Cholestasis | PRKCE; IRAK1; INS; MYD88; PRKCZ; TRAF6; PPARA; RXRA; IKBKB; |
| | PRKC1; NFKB2; MAP3K14; MAPK8; PRKD1; MAPK10; RELA; PRKCD; |
| | MAPK9; ABCB1; TRAF2; TLR4; TNF; INSR; IKBKG; RELB; MAP3K7; IL8; |
| | CHUK; NR1H2; TJP2; NFKB1; ESR1; SREBF1; FGFR4; JUN; IL1R1; |
| | PRKCA; IL6 |
| IGF-1 Signaling | IGF1; PRKCZ; ELK1; MAPK1; PTPN11; NEDD4; AKT2; PIK3CA; PRKC1; |
| | PTK2; FOS; PIK3CB; PIK3C3; MAPK8; IGFIR; IRS1; MAPK3; IGFBP7; |
| | KRAS; PIK3C2A; YWHAZ; PXN; RAF1; CASP9; MAP2K2; AKT1; PIK3R1; |
| | PDPKI; MAP2K1; IGFBP2; SFN; JUN; CYR61; AKT3; FOXO1; SRF; CTGF; |
| | RPS6KB1 |
| NRF2-mediated Oxidative | PRKCE; EP300; SOD2; PRKCZ; MAPK1; SQSTM1; NQO1; PIK3CA; |
| Stress Response | PRKC1; FOS; PIK3CB; PIK3C3; MAPK8; PRKD1; MAPK3; KRAS; PRKCD; |
| | GSTPI; MAPK9; FTL; NFE2L2; PIK3C2A; MAPK14; RAF1; MAP3K7; |
| | CREBBP; MAP2K2; AKT1; PIK3R1; MAP2K1; PPIB; JUN; KEAP1; GSK3B; |
| | ATF4; PRKCA; EIF2AK3; HSP90AA1 |
| Hepatic Fibrosis/Hepatic | EDN1; IGF1; KDR; FLT1; SMAD2; FGFR1; MET; PGF; SMAD3; EGFR; |
| Stellate Cell Activation | FAS; CSF1; NFKB2; BCL2; MYH9; IGFIR; IL6R; RELA; TLR4; PDGFRB; |
| | TNF; RELB; IL8; PDGFRA; NFKB1; TGFBR1; SMAD4; VEGFA; BAX; |
| | IL1R1; CCL2; HGF; MMPI; STAT1; IL6; CTGF; MMP9 |
| PPAR Signaling | EP300; INS; TRAF6; PPARA; RXRA; MAPK1; IKBKB; NCOR2; FOS; |
| | NFKB2; MAP3K14; STAT5B; MAPK3; NRIP1; KRAS; PPARG; RELA; |
| | STAT5A; TRAF2; PP ARGCI A; PDGFRB; TNF; INSR; RAF1; IKBKG; |
| | RELB; MAP3K7; CREBBP; MAP2K2; CHUK; PDGFRA; MAP2K1; NFKB1; |
| | JUN; IL1R1; HSP90AA1 |
| Fc Epsilon RI Signaling | PRKCE; RAC1; PRKCZ; LYN; MAPK1; RAC2; PTPN11; AKT2; PIK3CA; |
| | SYK; PRKC1; PIK3CB; PIK3C3; MAPK8; PRKD1; MAPK3; MAPK10; |
| | KRAS; MAPK13; PRKCD; MAPK9; PIK3C2A; BTK; MAPK14; TNF; RAF1; |
| | FYN; MAP2K2; AKT1; PIK3R1; PDPK1; MAP2K1; AKT3; VAV3; PRKCA |
| G-Protein Coupled Receptor | PRKCE; RAP1A; RGS16; MAPK1; GNAS; AKT2; IKBKB; PIK3CA; CREB1; |
| Signaling | GNAQ; NFKB2; CAMK2A; PIK3CB; PIK3C3; MAPK3; KRAS; RELA; SRC; |
| | PIK3C2A; RAF1; IKBKG; RELB; FYN; MAP2K2; AKT1; PIK3R1; CHUK; |
| | PDPK1; STAT3; MAP2K1; NFKB1; BRAF; ATF4; AKT3; PRKCA |
| Inositol Phosphate Metabolism | PRKCE; IRAK1; PRKAA2; EIF2AK2; PTEN; GRK6; MAPK1; PLK1; AKT2; |
| | PIK3CA; CDK8; PIK3CB; PIK3C3; MAPK8; MAPK3; PRKCD; PRKAA1; |
| | MAPK9; CDK2; PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; PIK3C3; MAPK8; |
| | CAV1; ABL1; MAPK3; KRAS; SRC; PIK3C2A; PDGFRB; RAF1; MAP2K2; |
| | JAK1; JAK2; PIK3R1; PDGFRA; STAT3; SPHK1; MAP2K1; MYC; JUN; |
| | CRKL; PRKCA; SRF; STAT1; SPHK2 |
| VEGF Signaling | ACTN4; ROCK1; KDR; FLT1; ROCK2; MAPK1; PGF; AKT2; PIK3CA; |
| | ARNT; PTK2; BCL2; PIK3CB; PIK3C3; BCL2L1; MAPK3; KRAS; HIF1A; |
| | NOS3; PIK3C2A; PXN; RAF1; MAP2K2; ELAVL1; AKT1; PIK3R1; |
| | MAP2K1; SFN; VEGFA; AKT3; FOXO1; PRKCA |
| Natural Killer Cell Signaling | PRKCE; RAC1; PRKCZ; MAPK1; RAC2; PTPN11; KIR2DL3; AKT2; |
| | PIK3CA; SYK; PRKC1; PIK3CB; PIK3C3; PRKD1; MAPK3; KRAS; PRKCD; |
| | PTPN6; PIK3C2A; LCK; RAF1; FYN; MAP2K2; PAK4; AKT1; PIK3R1; |
| | MAP2K1; PAK3; AKT3; VAV3; PRKCA |
| Cell Cycle: G1/S Checkpoint | HDAC4; SMAD3; SUV39H1; HDAC5; CDKN1B; BTRC; ATR; ABL1; E2F1; |
| Regulation | HDAC2; HDAC7A; RB1; HDAC11; HDAC9; CDK2; E2F2; HDAC3; TP53; |
| | CDKN1A; CCND1; E2F4; ATM; RBL2; SMAD4; CDKN2A; MYC; NRGI; |
| | GSK3B; RBL1; HDAC6 |
| T Cell Receptor Signaling | RAC1; ELK1; MAPK1; IKBKB; CBL; PIK3CA; FOS; NFKB2; PIK3CB; |
| | PIK3C3; MAPK8; MAPK3; KRAS; RELA; PIK3C2A; BTK; LCK; RAF1; |
| | IKBKG; RELB; FYN; MAP2K2; PIK3R1; CHUK; MAP2K1; NFKB1; ITK; |
| | BCL10; JUN; VAV3 |
| Death Receptor Signaling | CRADD; HSPB1; BID; BIRC4; TBK1; IKBKB; FADD; FAS; NFKB2; BCL2; |
| | MAP3K14; MAPK8; RIPK1; CASP8; DAXX; TNFRSF10B; RELA; TRAF2; |
| | TNF; IKBKG; RELB; CASP9; CHUK; APAF1; NFKB1; CASP2; BIRC2; |
| | CASP3;BIRC3 |
| FGF Signaling | RAC1; FGFR1; MET; MAPKAPK2; MAPK1; PTPN11; AKT2; PIK3CA; |
| | CREB1; PIK3CB; PIK3C3; MAPK8; MAPK3; MAPK13; PTPN6; PIK3C2A; |
| | MAPK14; RAF1; AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; STAT5B; |
| | PIK3CB; PIK3C3; GNB2L1; BCL2L1; MAPK3; ETS1; KRAS; RUNX1; |
| | PIM1; PIK3C2A; RAF1; MAP2K2; AKT1; JAK2; PIK3R1; STAT3; MAP2K1; |
| | CCNDI; AKT3; STAT1 |
| Amyotrophic Lateral Sclerosis | BID; IGF1; RAC1; BIRC4; PGF; CAPNS1; CAPN2; PIK3CA; BCL2; |
| Signaling | PIK3CB; PIK3C3; BCL2L1; CAPN1; PIK3C2A; TP53; CASP9; PIK3R1; |
| | RAB5A; CASP1; APAF1; VEGFA; BIRC2; BAX; AKT3; CASP3; BIRC3 |
| JAK/Stat Signaling | PTPN1; MAPK1; PTPN11; AKT2; PIK3CA; STAT5B; PIK3CB; PIK3C3; |
| | MAPK3; KRAS; SOCS1; STAT5A; PTPN6; PIK3C2A; RAF1; CDKN1A; |
| | MAP2K2; JAK1; AKT1; JAK2; PIK3R1; STAT3; MAP2K1; FRAP1; AKT3; |
| | STAT1 |
| Nicotinate and Nicotinamide | PRKCE; IRAK1; PRKAA2; EIF2AK2; GRK6; MAPK1; PLK1; AKT2; CDK8; |
| Metabolism | MAPK8; MAPK3; PRKCD; PRKAA1; PBEF1; MAPK9; CDK2; PIM1; |
| | DYRK1A; MAP2K2; MAP2K1; PAK3; NT5E; TTK; CSNK1A1; BRAF; SGK |
| Chemokine Signaling | CXCR4; ROCK2; MAPK1; PTK2; FOS; CFL1; GNAQ; CAMK2A; CXCL12; |
| | MAPK8; MAPK3; KRAS; MAPK13; RHOA; CCR3; SRC; PPPICC; |
| | MAPK14; NOX1; RAF1; MAP2K2; MAP2K1; JUN; CCL2; PRKCA |
| IL-2 Signaling | ELK1; MAPK1; PTPN11; AKT2; PIK3CA; SYK; FOS; STAT5B; PIK3CB; |
| | PIK3C3; MAPK8; MAPK3; KRAS; SOCS1; STAT5A; PIK3C2A; LCK; |
| | RAF1; MAP2K2; JAK1; AKT1; PIK3R1; MAP2K1; JUN; AKT3 |
| Synaptic Long Term | PRKCE; IGF1; PRKCZ; PRDX6; LYN; MAPK1; GNAS; PRKC1; GNAQ; |
| Depression | PPP2R1A; IGFIR; PRKD1; MAPK3; KRAS; GRN; PRKCD; NOS3; NOS2A; |
| | PPP2CA; YWHAZ; RAF1; MAP2K2; PPP2R5C; MAP2K1; PRKCA |
| Estrogen Receptor Signaling | TAF4B; EP300; CARMI; PCAF; MAPK1; NCOR2; SMARCA4; MAPK3; |
| | NRIP1; KRAS; SRC; NR3C1; HDAC3; PPARGC1A; RBM9; NCOA3; RAF1; |
| | CREBBP; MAP2K2; NCOA2; MAP2K1; PRKDC; ESR1; ESR2 |
| Protein Ubiquitination | TRAF6; SMURF1; BIRC4; BRCA1; UCHL1; NEDD4; CBL; UBE2I; BTRC; |
| Pathway | HSPA5; USP7; USP10; FBXW7; USP9X; STUB1; USP22; B2M; BIRC2; |
| | PARK2; USP8; USP1; VHL; HSP90AA1; BIRC3 |
| IL-10 Signaling | TRAF6; CCR1; ELK1; IKBKB; SP1; FOS; NFKB2; MAP3K14; MAPK8; |
| | MAPK13; RELA; MAPK14; TNF; IKBKG; RELB; MAP3K7; JAK1; CHUK; |
| | STAT3; NFKB1; JUN; IL1R1; IL6 |
| VDR/RXR Activation | PRKCE; EP300; PRKCZ; RXRA; GADD45A; HES1; NCOR2; SP1; PRKC1; |
| | CDKN1B; PRKD1; PRKCD; RUNX2; KLF4; YY1; NCOA3; CDKN1A; |
| | NCOA2; SPP1; LRP5; CEBPB; FOXO1; PRKCA |
| TGF-beta Signaling | EP300; SMAD2; SMURF1; MAPK1; SMAD3; SMAD1; FOS; MAPK8; |
| | MAPK3; KRAS; MAPK9; RUNX2; SERPINE1; RAF1; MAP3K7; CREBBP; |
| | MAP2K2; MAP2K1; TGFBR1; SMAD4; JUN; SMAD5 |
| Toll-like Receptor Signaling | IRAK1; EIF2AK2; MYD88; TRAF6; PPARA; ELK1; IKBKB; FOS; NFKB2; |
| | MAP3K14; MAPK8; MAPK13; RELA; TLR4; MAPK14; IKBKG; RELB; |
| | MAP3K7; CHUK; NFKB1; TLR2; JUN |
| p38 MAPK Signaling | HSPB1; IRAK1; TRAF6; MAPKAPK2; ELK1; FADD; FAS; CREB1; DDIT3; |
| | RPS6KA4; DAXX; MAPK13; TRAF2; MAPK14; TNF; MAP3K7; TGFBR1; |
| | MYC; ATF4; IL1R1; SRF; STAT1 |
| Neurotrophin/TRK Signaling | NTRK2; MAPK1; PTPN11; PIK3CA; CREB1; FOS; PIK3CB; PIK3C3; |
| | MAPK8; MAPK3; KRAS; PIK3C2A; RAF1; MAP2K2; AKT1; PIK3R1; |
| | PDPK1; MAP2K1; CDC42; JUN; ATF4 |
| FXR/RXR Activation | INS; PPARA; FASN; RXRA; AKT2; SDC1; MAPK8; APOB; MAPK10; |
| | PPARG; MTTP; MAPK9; PPARGC1A; TNF; CREBBP; AKT1; SREBF1; |
| | FGFR4; AKT3; FOXO1 |
| Synaptic Long Term | PRKCE; RAP1A; EP300; PRKCZ; MAPK1; CREB1; PRKC1; GNAQ; |
| Potentiation | CAMK2A; PRKD1; MAPK3; KRAS; PRKCD; PPPICC; RAF1; CREBBP; |
| | MAP2K2; MAP2K1; ATF4; PRKCA |
| Calcium Signaling | RAP1A; EP300; HDAC4; MAPK1; HDAC5; CREB1; CAMK2A; MYH9; |
| | MAPK3; HDAC2; HDAC7A; HDAC11; HDAC9; HDAC3; CREBBP; CALR; |
| | CAMKK2; ATF4; HDAC6 |
| EGF Signaling | ELK1; MAPK1; EGFR; PIK3CA; FOS; PIK3CB; PIK3C3; MAPK8; MAPK3; |
| | PIK3C2A; RAF1; JAK1; PIK3R1; STAT3; MAP2K1; JUN; PRKCA; SRF; |
| | STAT1 |
| Hypoxia Signaling in the | EDN1; PTEN; EP300; NQO1; UBE2I; CREB1; ARNT; HIF1A; SLC2A4; |
| Cardiovascular System | NOS3; TP53; LDHA; AKT1; ATM; VEGFA; JUN; ATF4; VHL; HSP90AA1 |
| LPS/IL-1 Mediated Inhibition | IRAK1; MYD88; TRAF6; PPARA; RXRA; ABCA1; MAPK8; ALDH1A1; |
| of RXR Function | GSTPI; MAPK9; ABCB1; TRAF2; TLR4; TNF; MAP3K7; NR1H2; SREBF1; |
| | JUN; IL1R1 |
| LXR/RXR Activation | FASN; RXRA; NCOR2; ABCA1; NFKB2; IRF3; RELA; NOS2A; TLR4; TNF; |
| | RELB; LDLR; NR1H2; NFKB1; SREBF1; IL1R1; CCL2; IL6; MMP9 |
| Amyloid Processing | PRKCE; CSNKIE; MAPK1; CAPNS1; AKT2; CAPN2; CAPN1; MAPK3; |
| | MAPK13; MAPT; MAPK14; AKT1; PSEN1; CSNK1A1; GSK3B; AKT3; APP |
| IL-4 Signaling | AKT2; PIK3CA; PIK3CB; PIK3C3; IRS1; KRAS; SOCS1; PTPN6; NR3C1; |
| | PIK3C2A; JAK1; AKT1; JAK2; PIK3R1; FRAP1; AKT3; RPS6KB1 |
| Cell Cycle: G2/M DNA | EP300; PCAF; BRCA1; GADD45A; PLK1; BTRC; CHEK1; ATR; CHEK2; |
| Damage Checkpoint | YWHAZ; TP53; CDKN1A; PRKDC; ATM; SFN; CDKN2A |
| Regulation | |
| Nitric Oxide Signaling in the | KDR; FLT1; PGF; AKT2; PIK3CA; PIK3CB; PIK3C3; CAV1; PRKCD; |
| Cardiovascular System | NOS3; PIK3C2A; AKT1; PIK3R1; VEGFA; AKT3; HSP90AA1 |
| Purine Metabolism | NME2; SMARCA4; MYH9; RRM2; ADAR; EIF2AK4; PKM2; ENTPD1; |
| | RAD51; RRM2B; TJP2; RAD51C; NT5E; POLD1; NME1 |
| cAMP-mediated Signaling | RAP1A; MAPK1; GNAS; CREB1; CAMK2A; MAPK3; SRC; RAF1; |
| | MAP2K2; STAT3; MAP2K1; BRAF; ATF4 |
| Mitochondrial Dysfunction | SOD2; MAPK8; CASP8; MAPK10; MAPK9; CASP9; PARK7; PSEN1; |
| Notch Signaling | PARK2; APP; CASP3 HES1; JAG1; NUMB; NOTCH4; ADAM17; NOTCH2; |
| | PSEN1; NOTCH3; NOTCH1; DLL4 |
| Endoplasmic Reticulum Stress | HSPA5; MAPK8; XBP1; TRAF2; ATF6; CASP9; ATF4; EIF2AK3; CASP3 |
| Pathway Pyrimidine | NME2; AICDA; RRM2; EIF2AK4; ENTPD1; RRM2B; NT5E; POLD1; NME1 |
| Metabolism | |
| Parkinson’s Signaling | UCHL1; MAPK8; MAPK13; MAPK14; CASP9; PARK7; PARK2; CASP3 |
| Cardiac & Beta Adrenergic | GNAS; GNAQ; PPP2R1A; GNB2L1; PPP2CA; PPP1CC; PPP2R5C |
| Signaling | |
| Glycolysis/Gluconeogenesis | HK2; GCK; GPI; ALDH1A1; PKM2; LDHA; HK1 |
| Interferon Signaling | IRF1; SOCS1; JAK1; JAK2; IFITM1; STAT1; IFIT3 |
| Sonic Hedgehog Signaling | ARRB2; SMO; GLI2; DYRK1A; GLI1; GSK3B; DYRKIB |
| Glycerophospholipid | PLD1; GRN; GPAM; YWHAZ; SPHK1; SPHK2 |
| Metabolism | |
| Phospholipid Degradation | PRDX6; PLD1; GRN; YWHAZ; SPHK1; SPHK2 |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision Repair | ERCC5; ERCC4; XPA; XPC; ERCC1 |
| Pathway | |
| Starch and Sucrose | UCHL1; HK2; GCK; GP1; HK1 |
| Metabolism | |
| Aminosugars Metabolism | NQO1; HK2; GCK; HK1 |
| Arachidonic Acid | PRDX6; GRN; YWHAZ; CYP1B1 |
| Metabolism | |
| Circadian Rhythm Signaling | CSNKIE; CREB1; ATF4; NR1D1 |
| Coagulation System | BDKRB1; F2R; SERPINE1; F3 |
| Dopamine Receptor | PPP2R1A; PPP2CA; PPPICC; PPP2R5C |
| Signaling | |
| Glutathione Metabolism | IDH2; GSTPI; ANPEP; IDH1 |
| Glycerolinid Metabolism | ALDH1A1; GPAM; SPHK1; SPHK2 |
| Linoleic Acid Metabolism | PRDX6; GRN; YWHAZ; CYP1B1 |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDXI; TYR |
| Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | EROIL; APEX1 |
| Metabolism of Xenobiotics | GSTPI; CYP1B1 |
| by Cytochrome p450 | |
| Methane Metabolism | PRDX6; PRDX1 |
| Phenylalanine Metabolism | PRDX6; PRDX1 |
| Propanoate Metabolism | ALDH1A1; LDHA |
| Selenoamino Acid | PRMT5; AHCY |
| Metabolism | |
| Sphingolipid Metabolism | SPHK1; SPHK2 |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated Oxidative | PRDX1 |
| Stress Response | |
| Pentose Phosphate | GP1 |
| Pathway | |
| Pentose and Glucuronate | UCHL1 |
| Interconversions | |
| Retinol Metabolism | ALDH1A1 |
| Riboflavin Metabolism | TYR |
| Tyrosine Metabolism | PRMT5, TYR |
| Ubiquinone Biosynthesis | PRMT5 |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cm2; Grk2; Trpa1; Pome; Cgrp; Crf; |
| | Pka; Era; Nr2b; TRPM5; Prkaca; Prkacb; Prkar1a; Prkar2a |
| Mitochondrial Function | AIF; CytC; SMAC (Diablo); Aifm-1; Aifm-2 |
| Developmental Neurology | BMP-4; Chordin (Chrd); Noggin (Nog); WNT (Wnt2; Wnt2b; Wnt3a; Wnt4; |
| | Wnt5a; Wnt6; Wnt7b; Wnt8b; Wnt9a; Wnt9b; Wnt10a; Wnt10b; Wnt16); beta- |
| | catenin; Dkk-1; Frizzled related proteins; Otx-2; Gbx2; FGF-8; Reelin; Dab1; |
| | unc-86 (Pou4f1 orBm3a); Numb; Reln |

In an aspect, the invention provides a method of individualized or personalized treatment of a genetic disease in a subject in need of such treatment comprising: (a) introducing one or more mutations ex vivo in a tissue, organ or a cell line, or in vivo in a transgenic non-human mammal, comprising delivering to cell(s) of the tissue, organ, cell or mammal a composition comprising the particle delivery system or the delivery system or the virus particle of any one of the above embodiments or the cell of any one of the above embodiments, wherein the specific mutations or precise sequence substitutions are or have been correlated to the genetic disease; (b) testing treatment(s) for the genetic disease on the cells to which the vector has been delivered that have the specific mutations or precise sequence substitutions correlated to the genetic disease; and (c) treating the subject based on results from the testing of treatment(s) of step (b).

### Infectious Diseases

In some embodiments, the composition, system(s) or component(s) thereof can be used to diagnose, prognose, treat, and/or prevent an infectious disease caused by a microorganism, such as bacteria, virus, fungi, parasites, or combinations thereof.

In some embodiments, the system(s) or component(s) thereof can be capable of targeting specific microorganism within a mixed population. Exemplary methods of such techniques are described in e.g. Gomaa A. A., Klumpe H. E., Luo M. L., Selle K, Barrangou R, Beisel C. L. 2014. Programmable removal of bacterial strains by use of genome-targeting composition, systems, mBio 5:e00928-13; Citorik R. J., Mimee M, Lu T. K. 2014. Sequence-specific antimicrobials using efficiently delivered RNA-guided nucleases. Nat Biotechnol 32:1141-1145, the teachings of which can be adapted for use with the compositions, systems, and components thereof described herein.

In some embodiments, the composition, system(s) and/or components thereof can be capable of targeting pathogenic and/or drug-resistant microorganisms, such as bacteria, virus, parasites, and fungi. In some embodiments, the composition, system(s) and/or components thereof can be capable of targeting and modifying one or more polynucleotides in a pathogenic microorganism such that the microorganism is less virulent, killed, inhibited, or is otherwise rendered incapable of causing disease and/or infecting and/or replicating in a host cell.

In some embodiments, the pathogenic bacteria that can be targeted and/or modified by the composition, system(s) and/or component(s) thereof described herein include, but are not limited to, those of the genus Actinomyces (e.g. A. israelii), Bacillus (e.g. B. anthracis, B. cereus), Bactereoides (e.g. B. fragilis), Bartonella (B. henselae, B. quintana), Bordetella (B. pertussis), Borrelia (e.g. B. burgdorferi, B. garinii, B. afzelii, and B. recurreentis), Brucella (e.g. B. abortus, B. canis, B. melitensis, and B. suis), Campylobacter (e.g. C. jejuni), Chlamydia (e.g. C. pneumoniae and C. trachomatis), Chlamydophila (e.g. C. psittaci), Clostridium (e.g. C. botulinum, C. difficile, C. perfringens. C. tetani), Corynebacterium (e.g. C. diptheriae), Enterococcus (e.g. E. Faecalis, E. faecium), Ehrlichia (E. canis and E. chaffensis) Escherichia (e.g. E. coli), Francisella (e.g. F. tularensis), Haemophilus (e.g. H. influenzae), Helicobacter (H. pylori), Klebsiella (E.g. K. pneumoniae), Legionella (e.g. L. pneumophila), Leptospira (e.g. L. interrogans, L. santarosai, L. weilii, L. noguchii), Listereia (e.g. L. monocytogenes), Mycobacterium (e.g. M. leprae, M. tuberculosis, M. ulcerans), Mycoplasma (M. pneumoniae), Neisseria (N. gonorrhoeae and N. menigitidis), Nocardia (e.g. N. asteroides), Pseudomonas (P. aeruginosa), Rickettsia (R. rickettsia), Salmonella (S. typhi and S. typhimurium), Shigella (S. sonnei and S. dysenteriae), Staphylococcus (S. aureus, S. epidermidis, and S. saprophyticus), Streptococcus (S. agalactiaee, S. pneumoniae, S. pyogenes), Treponema (T. pallidum), Ureaplasma (e.g. U. urealyticum), Vibrio (e.g. V. cholerae), Yersinia (e.g. Y. pestis, Y. enterocolitica, and Y. pseudotuberculosis).

In some embodiments, the pathogenic virus that can be targeted and/or modified by the composition, system(s) and/or component(s) thereof described herein include, but are not limited to, a double-stranded DNA virus, a partly double-stranded DNA virus, a single-stranded DNA virus, a positive single-stranded RNA virus, a negative single-stranded RNA virus, or a double stranded RNA virus. In some embodiments, the pathogenic virus can be from the family Adenoviridae (e.g. Adenovirus), Herpeesviridae (e.g. Herpes simplex, type 1, Herpes simplex, type 2, Varicella-zoster virus, Epstein-Barr virus, Human cytomegalovirus, Human herpesvirus, type 8), Papillomaviridae (e.g. Human papillomavirus), Polyomaviridae (e.g. BK virus, JC virus), Poxviridae (e.g. smallpox), Hepadnaviridae (e.g. Hepatitis B), Parvoviridae (e.g. Parvovirus B19), Astroviridae (e.g. Human astrovirus), Caliciviridae (e.g. Norwalk virus), Picornaviridae (e.g. coxsackievirus, hepatitis A virus, poliovirus, rhinovirus), Coronaviridae (e.g. Severe acute respiratory syndrome-related coronavirus, strains: Severe acute respiratory syndrome virus, Severe acute respiratory syndrome coronavirus 2 (COVID-19)), Flaviviridae (e.g. Hepatitis C virus, yellow fever virus, dengue virus, West Nile virus, TBE virus), Togaviridae (e.g. Rubella virus), Hepeviridae (e.g. Hepatitis E virus), Retroviridae (Human immunodeficiency virus (HIV)), Orthomyxoviridae (e.g. Influenza virus), Arenaviridae (e.g. Lassa virus), Bunyaviridae (e.g. Crimean-Congo hemorrhagic fever virus, Hantaan virus), Filoviridae (e.g. Ebola virus and Marburg virus), Paramyxoviridae (e.g. Measles virus, Mumps virus, Parainfluenza virus, Respiratory syncytial virus), Rhabdoviridae (Rabies virus), Hepatits D virus, Reoviridae (e.g. Rotavirus, Orbivirus, Coltivirus, Banna virus).

In some embodiments, the pathogenic fungi that can be targeted and/or modified by the composition, system(s) and/or component(s) thereof described herein include, but are not limited to, those of the genus Candida (e.g. C. albicans), Aspergillus (e.g. A. fumigatus, A. flavus, A. clavatus), Cryptococcus (e.g. C. neoformans, C. gattii), Histoplasma (e.g., H. capsulatum), Pneumocystis (e.g. P. jiroveecii), Stachybotrys (e.g. S. chartarum).

In some embodiments, the pathogenic parasites that can be targeted and/or modified by the composition, system(s) and/or component(s) thereof described herein include, but are not limited to, protozoa, helminths, and ectoparasites. In some embodiments, the pathogenic protozoa that can be targeted and/or modified by the composition, system(s) and/or component(s) thereof described herein include, but are not limited to, those from the groups Sarcodina (e.g. ameba such as Entamoeba), Mastigophora (e.g. flagellates such as Giardia and Leishmania), Cilophora (e.g. ciliates such as Balantidum), and sporozoa (e.g. plasmodium and cryptosporidium). In some embodiments, the pathogenic helminths that can be targeted and/or modified by the composition, system(s) and/or component(s) thereof described herein include, but are not limited to, flatworms (platyhelminths), thorny-headed worms (acanthoceephalins), and roundworms (nematodes). In some embodiments, the pathogenic ectoparasites that can be targeted and/or modified by the composition, system(s) and/or component(s) thereof described herein include, but are not limited to, ticks, fleas, lice, and mites.

In some embodiments, the pathogenic parasite that can be targeted and/or modified by the composition, system(s) and/or component(s) thereof described herein include, but are not limited to, Acanthamoeba spp., Balamuthia mandrillaris, Babesiosis spp. (e.g. Babesia B. divergens, B. bigemina, B. equi, B. microfti, B. duncani), Balantidiasis spp. (e.g. Balantidium coli), Blastocystis spp., Cryptosporidium spp., Cyclosporiasis spp. (e.g. Cyclospora cayetanensis), Dientamoebiasis spp. (e.g. Dientamoeba fragilis), Amoebiasis spp. (e.g. Entamoeba histolytica), Giardiasis spp. (e.g. Giardia lamblia), Isosporiasis spp. (e.g. Isospora belli), Leishmania spp., Naegleria spp. (e.g. Naegleria fowleri), Plasmodium spp. (e.g. Plasmodium falciparum, Plasmodium vivax, Plasmodium ovale curtisi, Plasmodium ovale wallikeri, Plasmodium malariae, Plasmodium knowlesi), Rhinosporidiosis spp. (e.g. Rhinosporidium seeberi), Sarcocystosis spp. (e.g. Sarcocystis bovihominis, Sarcocystis suihominis), Toxoplasma spp. (e.g. Toxoplasma gondii), Trichomonas spp. (e.g. Trichomonas vaginalis), Trypanosoma spp. (e.g. Trypanosoma brucei), Trypanosoma spp. (e.g. Trypanosoma cruzi), Tapeworm (e.g. Cestoda, Taenia multiceps, Taenia saginata, Taenia solium), Diphyllobothrium latum spp., Echinococcus spp. (e.g. Echinococcus granulosus, Echinococcus multilocularis, E. vogeli, E. oligarthrus), Hymenolepis spp. (e.g. Hymenolepis nana, Hymenolepis diminuta), Bertiella spp. (e.g. Bertiella mucronata, Bertiella studeri), Spirometra (e.g. Spirometra erinaceieuropaei), Clonorchis spp. (e.g. Clonorchis sinensis; Clonorchis viverrini), Dicrocoelium spp. (e.g. Dicrocoelium dendriticum), Fasciola spp. (e.g. Fasciola hepatica, Fasciola gigantica), Fasciolopsis spp. (e.g. Fasciolopsis buski), Metagonimus spp. (e.g. Metagonimus yokogawai), Metorchis spp. (e.g. Metorchis conjunctus), Opisthorchis spp. (e.g. Opisthorchis viverrini, Opisthorchis felineus), Clonorchis spp. (e.g. Clonorchis sinensis), Paragonimus spp. (e.g. Paragonimus westermani; Paragonimus africanus; Paragonimus caliensis; Paragonimus kellicotti; Paragonimus skrjabini; Paragonimus uterobilateralis), Schistosoma sp., Schistosoma spp. (e.g. Schistosoma mansoni, Schistosoma haematobium, Schistosoma japonicum, Schistosoma mekongi, and Schistosoma intercalatum), Echinostoma spp. (e.g. E. echinatum), Trichobilharzia spp. (e.g. Trichobilharzia regent), Ancylostoma spp. (e.g. Ancylostoma duodenale), Necator spp. (e.g. Necator americanus), Angiostrongylus spp., Anisakis spp., Ascaris spp. (e.g. Ascaris lumbricoides), Baylisascaris spp. (e.g. Baylisascaris procyonis), Brugia spp. (e.g. Brugia malayi, Brugia timori), Dioctophyme spp. (e.g. Dioctophyme renale), Dracunculus spp. (e.g. Dracunculus medinensis), Enterobius spp. (e.g. Enterobius vermicularis, Enterobius gregorii), Gnathostoma spp. (e.g. Gnathostoma spinigerum, Gnathostoma hispidum), Halicephalobus spp. (e.g. Halicephalobus gingivalis), Loa loa spp. (e.g. Loa loa filaria), Mansonella spp. (e.g. Mansonella streptocerca), Onchocerca spp. (e.g. Onchocerca volvulus), Strongyloides spp. (e.g. Strongyloides stercoralis), Thelazia spp. (e.g. Thelazia californiensis, Thelazia callipaeda), Toxocara spp. (e.g. Toxocara canis, Toxocara cati, Toxascaris leonine), Trichinella spp. (e.g. Trichinella spiralis, Trichinella britovi, Trichinella nelsoni, Trichinella nativa), Trichuris spp. (e.g. Trichuris trichiura, Trichuris vulpis), Wuchereria spp. (e.g. Wuchereria bancrofti), Dermatobia spp. (e.g. Dermatobia hominis), Tunga spp. (e.g. Tunga penetrans), Cochliomyia spp. (e.g. Cochliomyia hominivorax), Linguatula spp. (e.g. Linguatula serrata), Archiacanthocephala sp., Moniliformis sp. (e.g. Moniliformis moniliformis), Pediculus spp. (e.g. Pediculus humanus capitis, Pediculus humanus humanus), Pthirus spp. (e.g. Pthirus pubis), Arachnida spp. (e.g. Trombiculidae, Ixodidae, Argaside), Siphonaptera spp (e.g. Siphonaptera: Pulicinae), Cimicidae spp. (e.g. Cimex lectularius and Cimex hemipterus), Diptera spp., Demodex spp. (e.g. Demodex folliculorum brevis canis), Sarcoptes spp. (e.g. Sarcoptes scabiei), Dermanyssus spp. (e.g. Dermanyssus gallinae), Ornithonyssus spp. (e.g. Ornithonyssus sylviarum, Ornithonyssus bursa, Ornithonyssus bacoti), Laelaps spp. (e.g. Laelaps echidnina), Liponyssoides spp. (e.g. Liponyssoides sanguineus).

In some embodiments the gene targets can be any of those as set forth in Table 1 of Strich and Chertow. 2019. J. Clin. Microbio. 57:4 e01307-18, which is incorporated herein as if expressed in its entirety herein.

In some embodiments, the method can include delivering a composition, system, and/or component thereof to a pathogenic organism described herein, allowing the composition, system, and/or component thereof to specifically bind and modify one or more targets in the pathogenic organism, whereby the modification kills, inhibits, reduces the pathogenicity of the pathogenic organism, or otherwise renders the pathogenic organism non-pathogenic. In some embodiments, delivery of the composition, system, occurs in vivo (i.e. in the subject being treated). In some embodiments occurs by an intermediary, such as microorganism or phage that is non-pathogenic to the subject but is capable of transferring polynucleotides and/or infecting the pathogenic microorganism. In some embodiments, the intermediary microorganism can be an engineered bacteria, virus, or phage that contains the composition, system(s) and/or component(s) thereof and/or CRISPR-Cas vectors and/or vector systems. The method can include administering an intermediary microorganism containing the composition, system(s) and/or component(s) thereof and/or CRISPR-Cas vectors and/or vector systems to the subject to be treated. The intermediary microorganism can then produce the CRISPR-system and/or component thereof or transfer a composition, system, polynucleotide to the pathogenic organism. In embodiments, where the CRISPR-system and/or component thereof, vector, or vector system is transferred to the pathogenic microorganism, the composition, system, or component thereof is then produced in the pathogenic microorganism and modifies the pathogenic microorganism such that it is less virulent, killed, inhibited, or is otherwise rendered incapable of causing disease and/or infecting and/or replicating in a host or cell thereof.

In some embodiments, where the pathogenic microorganism inserts its genetic material into the host cell's genome (e.g. a virus), the composition, system, can be designed such that it modifies the host cell's genome such that the viral DNA or cDNA cannot be replicated by the host cell's machinery into a functional virus. In some embodiments, where the pathogenic microorganism inserts its genetic material into the host cell's genome (e.g. a virus), the composition, system can be designed such that it modifies the host cell's genome such that the viral DNA or cDNA is deleted from the host cell's genome.

It will be appreciated that inhibiting or killing the pathogenic microorganism, the disease and/or condition that its infection causes in the subject can be treated or prevented. Thus, also provided herein are methods of treating and/or preventing one or more diseases or symptoms thereof caused by any one or more pathogenic microorganisms, such as any of those described herein.

### Mitochondrial Diseases

Some of the most challenging mitochondrial disorders arise from mutations in mitochondrial DNA (mtDNA), a high copy number genome that is maternally inherited. In some embodiments, mtDNA mutations can be modified using a composition, system, described herein. In some embodiments, the mitochondrial disease that can be diagnosed, prognosed, treated, and/or prevented can be MELAS (mitochondrial myopathy encephalopathy, and lactic acidosis and stroke-like episodes), CPEO/PEO (chronic progressive external ophthalmoplegia syndrome/progressive external ophthalmoplegia), KSS (Kearns-Sayre syndrome), MIDD (maternally inherited diabetes and deafness), MERRF (myoclonic epilepsy associated with ragged red fibers), NIDDM (noninsulin-dependent diabetes mellitus), LHON (Leber hereditary optic neuropathy), LS (Leigh Syndrome) an aminoglycoside induced hearing disorder, NARP (neuropathy, ataxia, and pigmentary retinopathy), Extrapyramidal disorder with akinesia-rigidity, psychosis and SNHL, Nonsyndromic hearing loss a cardiomyopathy, an encephalomyopathy, Pearson's syndrome, or a combination thereof.

In some embodiments, the mtDNA of a subject can be modified in vivo or ex vivo. In some embodiments, where the mtDNA is modified ex vivo, after modification the cells containing the modified mitochondria can be administered back to the subject. In some embodiments, the composition, system, or component thereof can be capable of correcting an mtDNA mutation, or a combination thereof.

In some embodiments, at least one of the one or more mtDNA mutations is selected from the group consisting of. A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem repeats at positions 305-314 and/or 956-965, deletion at positions from 8,469-13,447, 4,308-14,874, and/or 4,398-14,822, 961ins/delC, the mitochondrial common deletion (e.g. mtDNA 4,977 bp deletion), and combinations thereof.

In some embodiments, the mitochondrial mutation can be any mutation as set forth in or as identified by use of one or more bioinformatic tools available at Mitomap available at mitomap.org. Such tools include, but are not limited to, “Variant Search, aka Market Finder”, Find Sequences for Any Haplogroup, aka “Sequence Finder”, “Variant Info”, “POLG Pathogenicity Prediction Server”, “MITOMASTER”, “Allele Search”, “Sequence and Variant Downloads”, “Data Downloads”. MitoMap contains reports of mutations in mtDNA that can be associated with disease and maintains a database of reported mitochondrial DNA Base Substitution Diseases: rRNA/tRNA mutations.

In some embodiments, the method includes delivering a composition, system, and/or a component thereof to a cell, and more specifically one or more mitochondria in a cell, allowing the composition, system, and/or component thereof to modify one or more target polynucleotides in the cell, and more specifically one or more mitochondria in the cell. The target polynucleotides can correspond to a mutation in the mtDNA, such as any one or more of those described herein. In some embodiments, the modification can alter a function of the mitochondria such that the mitochondria functions normally or at least is/are less dysfunctional as compared to an unmodified mitochondria. Modification can occur in vivo or ex vivo. Where modification is performed ex vivo, cells containing modified mitochondria can be administered to a subject in need thereof in an autologous or allogenic manner.

### Microbiome Modification

Microbiomes play important roles in health and disease. For example, the gut microbiome can play a role in health by controlling digestion, preventing growth of pathogenic microorganisms and have been suggested to influence mood and emotion. Imbalanced microbiomes can promote disease and are suggested to contribute to weight gain, unregulated blood sugar, high cholesterol, cancer, and other disorders. A healthy microbiome has a series of joint characteristics that can be distinguished from non-healthy individuals, thus detection and identification of the disease-associated microbiome can be used to diagnose and detect disease in an individual. The compositions, systems, and components thereof can be used to screen the microbiome cell population and be used to identify a disease associated microbiome. Cell screening methods utilizing compositions, systems, and components thereof are described elsewhere herein and can be applied to screening a microbiome, such as a gut, skin, vagina, and/or or al microbiome, of a subject.

In some embodiments, the microbe population of a microbiome in a subject can be modified using a composition, system, and/or component thereof described herein. In some embodiments, the composition, system, and/or component thereof can be used to identify and select one or more cell types in the microbiome and remove them from the microbiome population. Exemplary methods of selecting cells using a composition, system, and/or component thereof are described elsewhere herein. In this way the make-up or microorganism profile of the microbiome can be altered. In some embodiments, the alteration causes a change from a diseased microbiome composition to a healthy microbiome composition. In this way the ratio of one type or species of microorganism to another can be modified, such as going from a diseased ratio to a healthy ratio. In some embodiments, the cells selected are pathogenic microorganisms.

In some embodiments, the compositions and systems described herein can be used to modify a polynucleotide in a microorganism of a microbiome in a subject. In some embodiments, the microorganism is a pathogenic microorganism. In some embodiments, the microorganism is a commensal and non-pathogenic microorganism. Methods of modifying polynucleotides in a cell in the subject are described elsewhere herein and can be applied to these embodiments.

### Models of Diseases and Conditions

In an aspect, the invention provides a method of modeling a disease associated with a genomic locus in a eukaryotic organism or a non-human organism comprising manipulation of a target sequence within a coding, non-coding or regulatory element of said genomic locus comprising delivering a non-naturally occurring or engineered composition comprising a viral vector system comprising one or more viral vectors operably encoding a composition for expression thereof, wherein the composition comprises particle delivery system or the delivery system or the virus particle of any one of the above embodiments or the cell of any one of the above embodiments.

In one aspect, the invention provides a method of generating a model eukaryotic cell that can include one or more a mutated disease genes and/or infectious microorganisms. In some embodiments, a disease gene is any gene associated an increase in the risk of having or developing a disease. In some embodiments, the method includes (a) introducing one or more vectors into a eukaryotic cell, wherein the one or more vectors comprise a composition, system, and/or component thereof and/or a vector or vector system that is capable of driving expression of a composition, system, and/or component thereof including, but not limited to: a guide sequence optionally linked to a tracr mate sequence, a tracr sequence, one or more Cas effectors, and combinations thereof and (b) allowing a composition, system, or complex to bind to one or more target polynucleotides, e.g., to effect cleavage, nicking, or other modification of the target polynucleotide within said disease gene, wherein the composition, system, or complex is composed of one or more CRISPR-Cas effectors complexed with (1) one or more guide sequences that is/are hybridized to the target sequence(s) within the target polynucleotide(s), and optionally (2) the tracr mate sequence(s) that is/are hybridized to the tracr sequence(s), thereby generating a model eukaryotic cell comprising one or more mutated disease gene(s). Thus, in some embodiments the composition and system, contains nucleic acid molecules for and drives expression of one or more of: a Cas effector, a guide sequence linked to a tracr mate sequence, and a tracr sequence and/or a Homologous Recombination template and/or a stabilizing ligand if the Cas effector has a destabilization domain. In some embodiments, said cleavage comprises cleaving one or two strands at the location of the target sequence by the Cas effector(s). In some embodiments, nicking comprises nicking one or two strands at the location of the target sequence by the Cas effector(s). In some embodiments, said cleavage or nicking results in modified transcription of a target polynucleotide. In some embodiments, modification results in decreased transcription of the target polynucleotide. In some embodiments, the method further comprises repairing said cleaved ornicked target polynucleotide by homologous recombination with an recombination template polynucleotide, wherein said repair results in a mutation comprising an insertion, deletion, or substitution of one or more nucleotides of said target polynucleotide. In some embodiments, said mutation results in one or more amino acid changes in a protein expression from a gene comprising the target sequence.

The disease modeled can be any disease with a genetic or epigenetic component. In some embodiments, the disease modeled can be any as discussed elsewhere herein, including but not limited to any as set forth in Tables 10 and 11 herein.

### In Situ Disease Detection

The compositions, systems, and/or components thereof can be used for diagnostic methods of detection such as in CASFISH (see e.g. Deng et al. 2015. PNAS USA 112(38): 11870-11875), CRISPR-Live FISH (see e.g. Wang et al. 2020. Science; 365(6459):1301-1305), sm-FISH (Lee and Jefcoate. 2017. Front. Endocrinol. doi.org/10.3389/fendo.2017.00289), sequential FISH CRISPRainbow (Ma et al. Nat Biotechnol, 34 (2016), pp. 528-530), CRISPR-Sirius (Nat Methods, 15 (2018), pp. 928-931), Casilio (Cheng et al. Cell Res, 26 (2016), pp. 254-257), Halo-Tag based genomic loci visualization techniques (e.g. Deng et al. 2015. PNAS USA 112(38): 11870-11875; Knight et al., Science, 350 (2015), pp. 823-826), RNA-aptamer based methods (e.g. Ma et al., J Cell Biol, 214 (2016), pp. 529-537), molecular beacon-based methods (e.g. Zhao et al. Biomaterials, 100 (2016), pp. 172-183; Wu et al. Nucleic Acids Res (2018)), Quantum Dot-based systems (e.g. Ma et al. Anal Chem, 89 (2017), pp. 12896-12901), multiplexed methods (e.g. Ma et al., Proc Natl Acad Sci USA, 112 (2015), pp. 3002-3007; Fu et al. Nat Commun, 7 (2016), p. 11707; Ma et al. Nat Biotechnol, 34 (2016), pp. 528-530; Shao et al. Nucleic Acids Res, 44 (2016), Article e86); Wang et al. Sci Rep, 6 (2016), p. 26857), 9, and other in situ CRISPR-hybridization based methods (e.g. Chen et al. Cell, 155 (2013), pp. 1479-1491; Gu et al. Science, 359 (2018), pp. 1050-1055; Tanebaum et al. Cell, 159 (2014), pp. 635-646; Ye et al. Protein Cell, 8 (2017), pp. 853-855; Chen et al. Nat Commun, 9 (2018), p. 5065; Shao et al. ACS Synth Biol (2017); Fu et al. Nat Commun, 7 (2016), p. 11707; Shao et al. Nucleic Acids Res, 44 (2016), Article e86; Wang et al., Sci Rep, 6 (2016), p. 26857), all of which are incorporated by reference herein as if expressed in their entirety and whose teachings can be adapted to the compositions, systems, and components thereof described herein in view of the description herein.

In some embodiments, the composition, system, or component thereof can be used in a detection method, such as an in situ detection method described herein. In some embodiments, the composition, system, or component thereof can include a catalytically inactivate Cas effector described herein and use this system in detection methods such as fluorescence in situ hybridization (FISH) or any other described herein. In some embodiments, the inactivated Cas effector, which lacks the ability to produce DNA double-strand breaks may be fused with a marker, such as fluorescent protein, such as the enhanced green fluorescent protein (eEGFP) and co-expressed with small guide RNAs to target pericentric, centric and telomeric repeats in vivo. The dCas effector or system thereof can be used to visualize both repetitive sequences and individual genes in the human genome. Such new applications of labelled dCas effector and compositions, systems, thereof can be important in imaging cells and studying the functional nuclear architecture, especially in cases with a small nucleus volume or complex 3-D structures.

### Cell Selection

In some embodiments, the compositions, systems, and/or components thereof described herein can be used in a method to screen and/or select cells. In some embodiments, composition, system-based screening/selection method can be used to identify diseased cells in a cell population. In some embodiments, selection of the cells results in a modification in the cells such that the selected cells die. In this way, diseased cells can be identified, and removed from the healthy cell population. In some embodiments, the diseased cells can be a cancer cell, pre-cancerous cell, a virus or other pathogenic organism infected cells, or otherwise abnormal cell. In some embodiments, the modification can impart another detectable change in the cells to be selected (e.g. a functional change and/or genomic barcode) that facilitates selection of the desired cells. In some embodiments a negative selection scheme can be used to obtain a desired cell population. In these embodiments, the cells to be selected against are modified, thus can be removed from the cell population based on their death or identification or sorting based the detectable change imparted on the cells. Thus, in these embodiments, the remaining cells after selection are the desired cell population.

In some embodiments, a method of selecting one or more cell(s) containing a polynucleotide modification can include: introducing one or more composition, system(s) and/or components thereof, and/or vectors or vector systems into the cell(s), wherein the composition, system(s) and/or components thereof, and/or vectors or vector systems contains and/or is capable of expressing one or more of: a Cas effector, a guide sequence optionally linked to a tracr mate sequence, a tracr sequence, and an recombination template; wherein, for example that which is being expressed is within and expressed in vivo by the composition, system, vector or vector system and/or the recombination template comprises the one or more mutations that abolish Cas effector cleavage; allowing homologous recombination of the recombination template with the target polynucleotide in the cell(s) to be selected; allowing a composition, system, or complex to bind to a target polynucleotide to effect cleavage of the target polynucleotide within said gene, wherein the AAV-complex comprises the Cas effector complexed with (1) the guide sequence that is hybridized to the target sequence within the target polynucleotide, and (2) the tracr mate sequence that is hybridized to the tracr sequence, wherein binding of the complex to the target polynucleotide induces cell death or imparts some other detectable change to the cell, thereby allowing one or more cell(s) in which one or more mutations have been introduced to be selected. In some embodiments, the cell to be selected may be a eukaryotic cell. In some embodiments, the cell to be selected may be a prokaryotic cell. Selection of specific cells via the methods herein can be performed without requiring a selection marker or a two-step process that may include a counter-selection system.

### Therapeutic Agent Development

The compositions, systems, and components thereof described herein can be used to develop CRISPR-Cas-based and non-CRISPR-Cas-based biologically active agents, such as small molecule therapeutics. Thus, described herein are methods for developing a biologically active agent that modulates a cell function and/or signaling event associated with a disease and/or disease gene. In some embodiments, the method comprises (a) contacting a test compound with a diseased cell and/or a cell containing a disease gene cell; and (b) detecting a change in a readout that is indicative of a reduction or an augmentation of a cell signaling event or other cell functionality associated with said disease or disease gene, thereby developing said biologically active agent that modulates said cell signaling event or other functionality associated with said disease gene. In some embodiments, the diseased cell is a model cell described elsewhere herein. In some embodiments, the diseased cell is a diseased cell isolated from a subject in need of treatment. In some embodiments, the test compound is a small molecule agent. In some embodiments, test compound is a small molecule agent. In some embodiments, the test compound is a biologic molecule agent.

In some embodiments, the method involves developing a therapeutic based on the composition, system, described herein. In particular embodiments, the therapeutic comprises a Cas effector and/or a guide RNA capable of hybridizing to a target sequence of interest. In particular embodiments, the therapeutic is a vector or vector system that can contain a) a first regulatory element operably linked to a nucleotide sequence encoding the Cas effector protein(s); and b) a second regulatory element operably linked to one or more nucleotide sequences encoding one or more nucleic acid molecules comprising a guide RNA comprising a guide sequence, a direct repeat sequence; wherein components (a) and (b) are located on same or different vectors. In particular embodiments, the biologically active agent is a composition comprising a delivery system operably configured to deliver composition, system, or components thereof, and/or or one or more polynucleotide sequences, vectors, or vector systems containing or encoding said components into a cell and capable of forming a complex with the components of the composition and system herein, and wherein said complex is operable in the cell. In some embodiments, the complex can include the Cas effector protein(s) as described herein, guide RNA comprising the guide sequence, and a direct repeat sequence. In any such compositions, the delivery system can be a yeast system, a lipofection system, a microinjection system, a biolistic system, virosomes, liposomes, immunoliposomes, polycations, lipid:nucleic acid conjugates or artificial virions, or any other system as described herein. In particular embodiments, the delivery is via a particle, a nanoparticle, a lipid or a cell penetrating peptide (CPP).

Also described herein are methods for developing or designing a composition, system, optionally a composition, system, based therapy or therapeutic, comprising (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub)selected target sites needed to treat or otherwise modulate or manipulate a population, and optionally validating one or more of the (sub)selected target sites for an individual subject, optionally designing one or more gRNA recognizing one or more of said (sub)selected target sites.

In some embodiments, the method for developing or designing a gRNA for use in a composition, system, optionally a composition, system, based therapy or therapeutic, can include (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub)selected target sites needed to treat or otherwise modulate or manipulate a population, optionally validating one or more of the (sub)selected target sites for an individual subject, optionally designing one or more gRNA recognizing one or more of said (sub)selected target sites.

In some embodiments, the method for developing or designing a composition, system, optionally a composition, system, based therapy or therapeutic in a population, can include (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub)selected target sites needed to treat or otherwise modulate or manipulate a population, optionally validating one or more of the (sub)selected target sites for an individual subject, optionally designing one or more gRNA recognizing one or more of said (sub)selected target sites.

In some embodiments the method for developing or designing a gRNA for use in a composition, system, optionally a composition, system, based therapy or therapeutic in a population, can include (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub)selected target sites needed to treat or otherwise modulate or manipulate a population, optionally validating one or more of the (sub)selected target sites for an individual subject, optionally designing one or more gRNA recognizing one or more of said (sub)selected target sites.

In some embodiments, the method for developing or designing a composition, system, such as a composition, system, based therapy or therapeutic, optionally in a population; or for developing or designing a gRNA for use in a composition, system, optionally a composition, system, based therapy or therapeutic, optionally in a population, can include selecting a set of target sequences for one or more loci in a target population, wherein the target sequences do not contain variants occurring above a threshold allele frequency in the target population (i.e. platinum target sequences); removing from said selected (platinum) target sequences any target sequences having high frequency off-target candidates (relative to other (platinum) targets in the set) to define a final target sequence set; preparing one or more, such as a set of compositions, systems, based on the final target sequence set, optionally wherein a number of CRISP-Cas systems prepared is based (at least in part) on the size of a target population.

In certain embodiments, off-target candidates/off-targets, PFS, PAM restrictiveness, target cleavage efficiency, or effector protein specificity is identified or determined using a sequencing-based double-strand break (DSB) detection assay, such as described herein elsewhere. In certain embodiments, off-target candidates/off-targets are identified or determined using a sequencing-based double-strand break (DSB) detection assay, such as described herein elsewhere. In certain embodiments, off-targets, or of f target candidates have at least 1, preferably 1-3, mismatches or (distal) PFS or PAM mismatches, such as 1 or more, such as 1, 2, 3, or more (distal) PFS or PAM mismatches. In certain embodiments, sequencing-based DSB detection assay comprises labeling a site of a DSB with an adapter comprising a primer binding site, labeling a site of a DSB with a barcode or unique molecular identifier, or combination thereof, as described herein elsewhere.

It will be understood that the guide sequence of the gRNA is 100% complementary to the target site, i.e. does not comprise any mismatch with the target site. It will be further understood that “recognition” of an (off-)target site by a gRNA presupposes composition, system, functionality, i.e. an (off-)target site is only recognized by a gRNA if binding of the gRNA to the (off-)target site leads to composition, system, activity (such as induction of single or double strand DNA cleavage, transcriptional modulation, etc.).

In certain embodiments, the target sites having minimal sequence variation across a population are characterized by absence of sequence variation in at least 99%, preferably at least 99.9%, more preferably at least 99.99% of the population. In certain embodiments, optimizing target location comprises selecting target sequences or loci having an absence of sequence variation in at least 99%, %, preferably at least 99.9%, more preferably at least 99.99% of a population. These targets are referred to herein elsewhere also as “platinum targets”. In certain embodiments, said population comprises at least 1000 individuals, such as at least 5000 individuals, such as at least 10000 individuals, such as at least 50000 individuals.

In certain embodiments, the off-target sites are characterized by at least one mismatch between the off-target site and the gRNA. In certain embodiments, the off-target sites are characterized by at most five, preferably at most four, more preferably at most three mismatches between the off-target site and the gRNA. In certain embodiments, the off-target sites are characterized by at least one mismatch between the off-target site and the gRNA and by at most five, preferably at most four, more preferably at most three mismatches between the off-target site and the gRNA.

In certain embodiments, said minimal number of off-target sites across said population is determined for high-frequency haplotypes in said population. In certain embodiments, said minimal number of off-target sites across said population is determined for high-frequency haplotypes of the off-target site locus in said population. In certain embodiments, said minimal number of off-target sites across said population is determined for high-frequency haplotypes of the target site locus in said population. In certain embodiments, the high-frequency haplotypes are characterized by occurrence in at least 0.1% of the population.

In certain embodiments, the number of (sub)selected target sites needed to treat a population is estimated based on based low frequency sequence variation, such as low frequency sequence variation captured in large scale sequencing datasets. In certain embodiments, the number of (sub)selected target sites needed to treat a population of a given size is estimated.

In certain embodiments, the method further comprises obtaining genome sequencing data of a subject to be treated; and treating the subject with a composition, system, selected from the set of compositions, systems, wherein the composition, system, selected is based (at least in part) on the genome sequencing data of the individual. In certain embodiments, the ((sub)selected) target is validated by genome sequencing, preferably whole genome sequencing.

In certain embodiments, target sequences or loci as described herein are (further) selected based on optimization of one or more parameters, such as PFS or PAM type (natural or modified), PFS or PAM nucleotide content, PFS or PAM length, target sequence length, PFS or PAM restrictiveness, target cleavage efficiency, and target sequence position within a gene, a locus or other genomic region. Methods of optimization are discussed in greater detail elsewhere herein.

In certain embodiments, target sequences or loci as described herein are (further) selected based on optimization of one or more of target loci location, target length, target specificity, and PFS or PAM characteristics. As used herein, PFS or PAM characteristics may comprise for instance PFS or PAM sequence, PFS or PAM length, and/or PFS or PAM GC contents. In certain embodiments, optimizing PFS or PAM characteristics comprises optimizing nucleotide content of a PFS or PAM. In certain embodiments, optimizing nucleotide content of PFS or PAM is selecting a PFS or PAM with a motif that maximizes abundance in the one or more target loci, minimizes mutation frequency, or both. Minimizing mutation frequency can for instance be achieved by selecting PFS or PAM sequences devoid of or having low or minimal CpG.

In certain embodiments, the effector protein for each composition and system, in the set of compositions, systems, is selected based on optimization of one or more parameters selected from the group consisting of; effector protein size, ability of effector protein to access regions of high chromatin accessibility, degree of uniform enzyme activity across genomic targets, epigenetic tolerance, mismatch/budge tolerance, effector protein specificity, effector protein stability or half-life, effector protein immunogenicity or toxicity. Methods of optimization are discussed in greater detail elsewhere herein.

### Optimization of the Systems

The methods of the present invention can involve optimization of selected parameters or variables associated with the composition, system, and/or its functionality, as described herein further elsewhere. Optimization of the composition, system, in the methods as described herein may depend on the target(s), such as the therapeutic target or therapeutic targets, the mode or type of composition, system, modulation, such as composition, system, based therapeutic target(s) modulation, modification, or manipulation, as well as the delivery of the composition, system, components. One or more targets may be selected, depending on the genotypic and/or phenotypic outcome. For instance, one or more therapeutic targets may be selected, depending on (genetic) disease etiology or the desired therapeutic outcome. The (therapeutic) target(s) may be a single gene, locus, or other genomic site, or may be multiple genes, loci or other genomic sites. As is known in the art, a single gene, locus, or other genomic site may be targeted more than once, such as by use of multiple gRNAs.

The activity of the composition and/or system, such as CRISPR-Cas system-based therapy or therapeutics may involve target disruption, such as target mutation, such as leading to gene knockout. The activity of the composition and/or system, such as CRISPR-Cas system-based therapy or therapeutics may involve replacement of particular target sites, such as leading to target correction. CRISPR-Cas system-based therapy or therapeutics may involve removal of particular target sites, such as leading to target deletion. The activity of the composition and/or system, such as CRISPR-Cas system-based therapy or therapeutics may involve modulation of target site functionality, such as target site activity or accessibility, leading for instance to (transcriptional and/or epigenetic) gene or genomic region activation or gene or genomic region silencing. The skilled person will understand that modulation of target site functionality may involve CRISPR effector mutation (such as for instance generation of a catalytically inactive CRISPR effector) and/or functionalization (such as for instance fusion of the CRISPR effector with a heterologous functional domain, such as a transcriptional activator or repressor), as described herein elsewhere.

Accordingly, in an aspect, the invention relates to a method as described herein, comprising selection of one or more (therapeutic) target, selecting one or more functionality of the composition and/or system, and optimization of selected parameters or variables associated with the CRISPR-Cas system and/or its functionality. In a related aspect, the invention relates to a method as described herein, comprising (a) selecting one or more (therapeutic) target loci, (b) selecting one or more CRISPR-Cas system functionalities, (c) optionally selecting one or more modes of delivery, and preparing, developing, or designing a CRISPR-Cas system selected based on steps (a)-(c).

In certain embodiments, the functionality of the composition and/or system comprises genomic mutation. In certain embodiments, the functionality of the composition and/or system comprises single genomic mutation. In certain embodiments, the functionality of the composition and/or system functionality comprises multiple genomic mutation. In certain embodiments, the functionality of the composition and/or system comprises gene knockout. In certain embodiments, the functionality of the composition and/or system comprises single gene knockout. In certain embodiments, the functionality of the composition and/or system comprises multiple gene knockout. In certain embodiments, the functionality of the composition and/or system comprises gene correction. In certain embodiments, the functionality of the composition and/or system comprises single gene correction. In certain embodiments, the functionality of the composition and/or system comprises multiple gene correction. In certain embodiments, the functionality of the composition and/or system comprises genomic region correction. In certain embodiments, the functionality of the composition and/or system comprises single genomic region correction. In certain embodiments, the functionality of the composition and/or system comprises multiple genomic region correction. In certain embodiments, the functionality of the composition and/or system comprises gene deletion. In certain embodiments, the functionality of the composition and/or system comprises single gene deletion. In certain embodiments, the functionality of the composition and/or system comprises multiple gene deletion. In certain embodiments, the functionality of the composition and/or system comprises genomic region deletion. In certain embodiments, the functionality of the composition and/or system comprises single genomic region deletion. In certain embodiments, the functionality of the composition and/or system comprises multiple genomic region deletion. In certain embodiments, the functionality of the composition and/or system comprises modulation of gene or genomic region functionality. In certain embodiments, the functionality of the composition and/or system comprises modulation of single gene or genomic region functionality. In certain embodiments, the functionality of the composition and/or system comprises modulation of multiple gene or genomic region functionality. In certain embodiments, the functionality of the composition and/or system comprises gene or genomic region functionality, such as gene or genomic region activity. In certain embodiments, the functionality of the composition and/or system comprises single gene or genomic region functionality, such as gene or genomic region activity. In certain embodiments, the functionality of the composition and/or system comprises multiple gene or genomic region functionality, such as gene or genomic region activity. In certain embodiments, the functionality of the composition and/or system comprises modulation gene activity or accessibility optionally leading to transcriptional and/or epigenetic gene or genomic region activation or gene or genomic region silencing. In certain embodiments, the functionality of the composition and/or system comprises modulation single gene activity or accessibility optionally leading to transcriptional and/or epigenetic gene or genomic region activation or gene or genomic region silencing. In certain embodiments, the functionality of the composition and/or system comprises modulation multiple gene activity or accessibility optionally leading to transcriptional and/or epigenetic gene or genomic region activation or gene or genomic region silencing.

Optimization of selected parameters or variables in the methods as described herein may result in optimized or improved the system, such as CRISPR-Cas system-based therapy or therapeutic, specificity, efficacy, and/or safety. In certain embodiments, one or more of the following parameters or variables are taken into account, are selected, or are optimized in the methods of the invention as described herein: Cas protein allosteric interactions, Cas protein functional domains and functional domain interactions, CRISPR effector specificity, gRNA specificity, CRISPR-Cas complex specificity, PFS or PAM restrictiveness, PFS or PAM type (natural or modified), PFS or PAM nucleotide content, PFS or PAM length, CRISPR effector activity, gRNA activity, CRISPR-Cas complex activity, target cleavage efficiency, target site selection, target sequence length, ability of effector protein to access regions of high chromatin accessibility, degree of uniform enzyme activity across genomic targets, epigenetic tolerance, mismatch/budge tolerance, CRISPR effector stability, CRISPR effector mRNA stability, gRNA stability, CRISPR-Cas complex stability, CRISPR effector protein or mRNA immunogenicity or toxicity, gRNA immunogenicity or toxicity, CRISPR-Cas complex immunogenicity or toxicity, CRISPR effector protein or mRNA dose or titer, gRNA dose or titer, CRISPR-Cas complex dose or titer, CRISPR effector protein size, CRISPR effector expression level, gRNA expression level, CRISPR-Cas complex expression level, CRISPR effector spatiotemporal expression, gRNA spatiotemporal expression, CRISPR-Cas complex spatiotemporal expression.

By means of example, and without limitation, parameter or variable optimization may be achieved as follows. CRISPR effector specificity may be optimized by selecting the most specific CRISPR effector. This may be achieved for instance by selecting the most specific CRISPR effector ortholog or by specific CRISPR effector mutations which increase specificity. gRNA specificity may be optimized by selecting the most specific gRNA. This can be achieved for instance by selecting gRNA having low homology, i.e. at least one or preferably more, such as at least 2, or preferably at least 3, mismatches to off-target sites. CRISPR-Cas complex specificity may be optimized by increasing CRISPR effector specificity and/or gRNA specificity as above. PFS or PAM restrictiveness may be optimized by selecting a CRISPR effector having to most restrictive PFS or PAM recognition. This can be achieved for instance by selecting a CRISPR effector ortholog having more restrictive PFS or PAM recognition or by specific CRISPR effector mutations which increase or alter PFS or PAM restrictiveness. PFS or PAM type may be optimized for instance by selecting the appropriate CRISPR effector, such as the appropriate CRISPR effector recognizing a desired PFS or PAM type. The CRISPR effector or PFS or PAM type may be naturally occurring or may for instance be optimized based on CRISPR effector mutants having an altered PFS or PAM recognition, or PFS or PAM recognition repertoire. PFS or PAM nucleotide content may for instance be optimized by selecting the appropriate CRISPR effector, such as the appropriate CRISPR effector recognizing a desired PFS or PAM nucleotide content. The CRISPR effector or PFS or PAM type may be naturally occurring or may for instance be optimized based on CRISPR effector mutants having an altered PFS or PAM recognition, or PAM recognition repertoire. PFS or PAM length may for instance be optimized by selecting the appropriate CRISPR effector, such as the appropriate CRISPR effector recognizing a desired PFS or PAM nucleotide length. The CRISPR effector or PFS or PAM type may be naturally occurring or may for instance be optimized based on CRISPR effector mutants having an altered PFS or PAM recognition, or PFS or PAM recognition repertoire.

Target length or target sequence length may be optimized, for instance, by selecting the appropriate CRISPR effector, such as the appropriate CRISPR effector recognizing a desired target or target sequence nucleotide length. Alternatively, or in addition, the target (sequence) length may be optimized by providing a target having a length deviating from the target (sequence) length typically associated with the CRISPR effector, such as the naturally occurring CRISPR effector. The CRISPR effector or target (sequence) length may be naturally occurring or may for instance be optimized based on CRISPR effector mutants having an altered target (sequence) length recognition, or target (sequence) length recognition repertoire. For instance, increasing or decreasing target (sequence) length may influence target recognition and/or off-target recognition. CRISPR effector activity may be optimized by selecting the most active CRISPR effector. This may be achieved for instance by selecting the most active CRISPR effector ortholog or by specific CRISPR effector mutations which increase activity. The ability of the CRISPR effector protein to access regions of high chromatin accessibility, may be optimized by selecting the appropriate CRISPR effector or mutant thereof, and can consider the size of the CRISPR effector, charge, or other dimensional variables etc. The degree of uniform CRISPR effector activity may be optimized by selecting the appropriate CRISPR effector or mutant thereof, and can consider CRISPR effector specificity and/or activity, PFS or PAM specificity, target length, mismatch tolerance, epigenetic tolerance, CRISPR effector and/or gRNA stability and/or half-life, CRISPR effector and/or gRNA immunogenicity and/or toxicity, etc. gRNA activity may be optimized by selecting the most active gRNA. In some embodiments, this can be achieved by increasing gRNA stability through RNA modification. CRISPR-Cas complex activity may be optimized by increasing CRISPR effector activity and/or gRNA activity as above.

The target site selection may be optimized by selecting the optimal position of the target site within a gene, locus or other genomic region. The target site selection may be optimized by optimizing target location comprises selecting a target sequence with a gene, locus, or other genomic region having low variability. This may be achieved for instance by selecting a target site in an early and/or conserved exon or domain (i.e. having low variability, such as polymorphisms, within a population).

In certain embodiments, optimizing target (sequence) length comprises selecting a target sequence within one or more target loci between 5 and 25 nucleotides. In certain embodiments, a target sequence is 20 nucleotides.

In certain embodiments, optimizing target specificity comprises selecting targets loci that minimize off-target candidates.

In some embodiments, the target site may be selected by minimization of off-target effects (e.g. off-targets qualified as having 1-5, 1-4, or preferably 1-3 mismatches compared to target and/or having one or more PFS or PAM mismatches, such as distal PFS or PAM mismatches), preferably also considering variability within a population. CRISPR effector stability may be optimized by selecting CRISPR effector having appropriate half-life, such as preferably a short half-life while still capable of maintaining sufficient activity. In some embodiments, this can be achieved by selecting an appropriate CRISPR effector ortholog having a specific half-life or by specific CRISPR effector mutations or modifications which affect half-life or stability, such as inclusion (e.g. fusion) of stabilizing or destabilizing domains or sequences. CRISPR effector mRNA stability may be optimized by increasing or decreasing CRISPR effector mRNA stability. In some embodiments, this can be achieved by increasing or decreasing CRISPR effector mRNA stability through mRNA modification. gRNA stability may be optimized by increasing or decreasing gRNA stability. In some embodiments, this can be achieved by increasing or decreasing gRNA stability through RNA modification. CRISPR-Cas complex stability may be optimized by increasing or decreasing CRISPR effector stability and/or gRNA stability as above. CRISPR effector protein or mRNA immunogenicity or toxicity may be optimized by decreasing CRISPR effector protein or mRNA immunogenicity or toxicity. In some embodiments, this can be achieved by mRNA or protein modifications. Similarly, in case of DNA based expression systems, DNA immunogenicity or toxicity may be decreased. gRNA immunogenicity or toxicity may be optimized by decreasing gRNA immunogenicity or toxicity. In some embodiments, this can be achieved by gRNA modifications. Similarly, in case of DNA based expression systems, DNA immunogenicity or toxicity may be decreased. CRISPR-Cas complex immunogenicity or toxicity may be optimized by decreasing CRISPR effector immunogenicity or toxicity and/or gRNA immunogenicity or toxicity as above, or by selecting the least immunogenic or toxic CRISPR effector/gRNA combination. Similarly, in the case of DNA based expression systems, DNA immunogenicity or toxicity may be decreased. CRISPR effector protein or mRNA dose or titer may be optimized by selecting dosage or titer to minimize toxicity and/or maximize specificity and/or efficacy. gRNA dose or titer may be optimized by selecting dosage or titer to minimize toxicity and/or maximize specificity and/or efficacy. CRISPR-Cas complex dose or titer may be optimized by selecting dosage or titer to minimize toxicity and/or maximize specificity and/or efficacy. CRISPR effector protein size may be optimized by selecting minimal protein size to increase efficiency of delivery, in particular for virus mediated delivery. CRISPR effector, gRNA, or CRISPR-Cas complex expression level may be optimized by limiting (or extending) the duration of expression and/or limiting (or increasing) expression level. This may be achieved for instance by using self-inactivating compositions, systems, such as including a self-targeting (e.g. CRISPR effector targeting) gRNA, by using viral vectors having limited expression duration, by using appropriate promoters for low (or high) expression levels, by combining different delivery methods for individual CRISP-Cas system components, such as virus mediated delivery of CRISPR-effector encoding nucleic acid combined with non-virus mediated delivery of gRNA, or virus mediated delivery of gRNA combined with non-virus mediated delivery of CRISPR effector protein or mRNA. CRISPR effector, gRNA, or CRISPR-Cas complex spatiotemporal expression may be optimized by appropriate choice of conditional and/or inducible expression systems, including controllable CRISPR effector activity optionally a destabilized CRISPR effector and/or a split CRISPR effector, and/or cell- or tissue-specific expression systems.

In an aspect, the invention relates to a method as described herein, comprising selection of one or more (therapeutic) target, selecting the functionality of the composition and/or system, selecting CRISPR-Cas system mode of delivery, selecting CRISPR-Cas system delivery vehicle or expression system, and optimization of selected parameters or variables associated with the CRISPR-Cas system and/or its functionality, optionally wherein the parameters or variables are one or more selected from CRISPR effector specificity, gRNA specificity, CRISPR-Cas complex specificity, PFS or PAM restrictiveness, PFS or PAM type (natural or modified), PFS or PAM nucleotide content, PFS or PAM length, CRISPR effector activity, gRNA activity, CRISPR-Cas complex activity, target cleavage efficiency, target site selection, target sequence length, ability of effector protein to access regions of high chromatin accessibility, degree of uniform enzyme activity across genomic targets, epigenetic tolerance, mismatch/budge tolerance, CRISPR effector stability, CRISPR effector mRNA stability, gRNA stability, CRISPR-Cas complex stability, CRISPR effector protein or mRNA immunogenicity or toxicity, gRNA immunogenicity or toxicity, CRISPR-Cas complex immunogenicity or toxicity, CRISPR effector protein or mRNA dose or titer, gRNA dose or titer, CRISPR-Cas complex dose or titer, CRISPR effector protein size, CRISPR effector expression level, gRNA expression level, CRISPR-Cas complex expression level, CRISPR effector spatiotemporal expression, gRNA spatiotemporal expression, CRISPR-Cas complex spatiotemporal expression.

In an aspect, the invention relates to a method as described herein, comprising selecting one or more (therapeutic) target, selecting one or more the functionality of the composition and/or system, selecting one or more CRISPR-Cas system mode of delivery, selecting one or more delivery vehicle or expression system, and optimization of selected parameters or variables associated with the CRISPR-Cas system and/or its functionality, wherein specificity, efficacy, and/or safety are optimized, and optionally wherein optimization of specificity comprises optimizing one or more parameters or variables selected from CRISPR effector specificity, gRNA specificity, CRISPR-Cas complex specificity, PFS or PAM restrictiveness, PFS or PAM type (natural or modified), PFS or PAM nucleotide content, PFS or PAM length, wherein optimization of efficacy comprises optimizing one or more parameters or variables selected from CRISPR effector activity, gRNA activity, CRISPR-Cas complex activity, target cleavage efficiency, target site selection, target sequence length, CRISPR effector protein size, ability of effector protein to access regions of high chromatin accessibility, degree of uniform enzyme activity across genomic targets, epigenetic tolerance, mismatch/budge tolerance, and wherein optimization of safety comprises optimizing one or more parameters or variables selected from CRISPR effector stability, CRISPR effector mRNA stability, gRNA stability, CRISPR-Cas complex stability, CRISPR effector protein or mRNA immunogenicity or toxicity, gRNA immunogenicity or toxicity, CRISPR-Cas complex immunogenicity or toxicity, CRISPR effector protein or mRNA dose or titer, gRNA dose or titer, CRISPR-Cas complex dose or titer, CRISPR effector expression level, gRNA expression level, CRISPR-Cas complex expression level, CRISPR effector spatiotemporal expression, gRNA spatiotemporal expression, CRISPR-Cas complex spatiotemporal expression.

In an aspect, the invention relates to a method as described herein, comprising optionally selecting one or more (therapeutic) target, optionally selecting one or more the functionality of the composition and/or system, optionally selecting one or more mode of delivery, optionally selecting one or more delivery vehicle or expression system, and optimization of selected parameters or variables associated with the system and/or its functionality, wherein specificity, efficacy, and/or safety are optimized, and optionally wherein optimization of specificity comprises optimizing one or more parameters or variables selected from CRISPR effector specificity, gRNA specificity, CRISPR-Cas complex specificity, PFS or PAM restrictiveness, PFS or PAM type (natural or modified), PFS or PAM nucleotide content, PFS or PAM length, wherein optimization of efficacy comprises optimizing one or more parameters or variables selected from CRISPR effector activity, gRNA activity, CRISPR-Cas complex activity, target cleavage efficiency, target site selection, target sequence length, CRISPR effector protein size, ability of effector protein to access regions of high chromatin accessibility, degree of uniform enzyme activity across genomic targets, epigenetic tolerance, mismatch/budge tolerance, and wherein optimization of safety comprises optimizing one or more parameters or variables selected from CRISPR effector stability, CRISPR effector mRNA stability, gRNA stability, CRISPR-Cas complex stability, CRISPR effector protein or mRNA immunogenicity or toxicity, gRNA immunogenicity or toxicity, CRISPR-Cas complex immunogenicity or toxicity, CRISPR effector protein or mRNA dose or titer, gRNA dose or titer, CRISPR-Cas complex dose or titer, CRISPR effector expression level, gRNA expression level, CRISPR-Cas complex expression level, CRISPR effector spatiotemporal expression, gRNA spatiotemporal expression, CRISPR-Cas complex spatiotemporal expression.

In an aspect, the invention relates to a method as described herein, comprising optimization of selected parameters or variables associated with the system and/or its functionality, wherein specificity, efficacy, and/or safety are optimized, and optionally wherein optimization of specificity comprises optimizing one or more parameters or variables selected from CRISPR effector specificity, gRNA specificity, CRISPR-Cas complex specificity, PFS or PAM restrictiveness, PFS or PAM type (natural or modified), PFS or PAM nucleotide content, PFS or PAM length, wherein optimization of efficacy comprises optimizing one or more parameters or variables selected from CRISPR effector activity, gRNA activity, CRISPR-Cas complex activity, target cleavage efficiency, target site selection, target sequence length, CRISPR effector protein size, ability of effector protein to access regions of high chromatin accessibility, degree of uniform enzyme activity across genomic targets, epigenetic tolerance, mismatch/budge tolerance, and wherein optimization of safety comprises optimizing one or more parameters or variables selected from CRISPR effector stability, CRISPR effector mRNA stability, gRNA stability, CRISPR-Cas complex stability, CRISPR effector protein or mRNA immunogenicity or toxicity, gRNA immunogenicity or toxicity, CRISPR-Cas complex immunogenicity or toxicity, CRISPR effector protein or mRNA dose or titer, gRNA dose or titer, CRISPR-Cas complex dose or titer, CRISPR effector expression level, gRNA expression level, CRISPR-Cas complex expression level, CRISPR effector spatiotemporal expression, gRNA spatiotemporal expression, CRISPR-Cas complex spatiotemporal expression.

It will be understood that the parameters or variables to be optimized as well as the nature of optimization may depend on the (therapeutic) target, the functionality of the composition and/or system, the system mode of delivery, and/or the CRISPR-Cas system delivery vehicle or expression system.

In an aspect, the invention relates to a method as described herein, comprising optimization of gRNA specificity at the population level. Preferably, said optimization of gRNA specificity comprises minimizing gRNA target site sequence variation across a population and/or minimizing gRNA off-target incidence across a population.

In some embodiments, optimization can result in selection of a CRISPR-Cas effector that is naturally occurring or is modified. In some embodiments, optimization can result in selection of a CRISPR-Cas effector that has nuclease, nickase, deaminase, transposase, and/or has one or more effector functionalities deactivated or eliminated. In some embodiments, optimizing a PFS or PAM specificity can include selecting a CRISPR-Cas effector with a modified PFS or PAM specificity. In some embodiments, optimizing can include selecting a CRISPR-Cas effector having a minimal size. In certain embodiments, optimizing effector protein stability comprises selecting an effector protein having a short half-life while maintaining sufficient activity, such as by selecting an appropriate CRISPR effector ortholog having a specific half-life or stability. In certain embodiments, optimizing immunogenicity or toxicity comprises minimizing effector protein immunogenicity or toxicity by protein modifications. In certain embodiments, optimizing functional specific comprises selecting a protein effector with reduced tolerance of mismatches and/or bulges between the guide RNA and one or more target loci.

In certain embodiments, optimizing efficacy comprises optimizing overall efficiency, epigenetic tolerance, or both. In certain embodiments, maximizing overall efficiency comprises selecting an effector protein with uniform enzyme activity across target loci with varying chromatin complexity, selecting an effector protein with enzyme activity limited to areas of open chromatin accessibility. In certain embodiments, chromatin accessibility is measured using one or more of ATAC-seq, or a DNA-proximity ligation assay. In certain embodiments, optimizing epigenetic tolerance comprises optimizing methylation tolerance, epigenetic mark competition, or both. In certain embodiments, optimizing methylation tolerance comprises selecting an effector protein that modify methylated DNA. In certain embodiments, optimizing epigenetic tolerance comprises selecting an effector protein unable to modify silenced regions of a chromosome, selecting an effector protein able to modify silenced regions of a chromosome, or selecting target loci not enriched for epigenetic markers

In certain embodiments, selecting an optimized guide RNA comprises optimizing gRNA stability, gRNA immunogenicity, or both, or other gRNA associated parameters or variables as described herein elsewhere.

In certain embodiments, optimizing gRNA stability and/or gRNA immunogenicity comprises RNA modification, or other gRNA associated parameters or variables as described herein elsewhere. In certain embodiments, the modification comprises removing 1-3 nucleotides form the 3′ end of a target complementarity region of the gRNA. In certain embodiments, modification comprises an extended gRNA and/or trans RNA/DNA element that create stable structures in the gRNA that compete with gRNA base pairing at a target of off-target loci, or extended complimentary nucleotides between the gRNA and target sequence, or both.

In certain embodiments, the mode of delivery comprises delivering gRNA and/or CRISPR effector protein, delivering gRNA and/or CRISPR effector mRNA, or delivery gRNA and/or CRISPR effector as a DNA based expression system. In certain embodiments, the mode of delivery further comprises selecting a delivery vehicle and/or expression systems from the group consisting of liposomes, lipid particles, nanoparticles, biolistics, or viral-based expression/delivery systems. In certain embodiments, expression is spatiotemporal expression is optimized by choice of conditional and/or inducible expression systems, including controllable CRISPR effector activity optionally a destabilized CRISPR effector and/or a split CRISPR effector, and/or cell- or tissue-specific expression system.

The methods as described herein may further involve selection of the mode of delivery. In certain embodiments, gRNA (and tracr, if and where needed, optionally provided as a sgRNA) and/or CRISPR effector protein are or are to be delivered. In certain embodiments, gRNA (and tracr, if and where needed, optionally provided as a sgRNA) and/or CRISPR effector mRNA are or are to be delivered. In certain embodiments, gRNA (and tracr, if and where needed, optionally provided as a sgRNA) and/or CRISPR effector provided in a DNA-based expression system are or are to be delivered. In certain embodiments, delivery of the individual system components comprises a combination of the above modes of delivery. In certain embodiments, delivery comprises delivering gRNA and/or CRISPR effector protein, delivering gRNA and/or CRISPR effector mRNA, or delivering gRNA and/or CRISPR effector as a DNA based expression system.

The methods as described herein may further involve selection of the CRISPR-Cas system delivery vehicle and/or expression system. Delivery vehicles and expression systems are described herein elsewhere. By means of example, delivery vehicles of nucleic acids and/or proteins include nanoparticles, liposomes, etc. Delivery vehicles for DNA, such as DNA-based expression systems include for instance biolistics, viral based vector systems (e.g. adenoviral, AAV, lentiviral), etc. the skilled person will understand that selection of the mode of delivery, as well as delivery vehicle or expression system may depend on for instance the cell or tissues to be targeted. In certain embodiments, the delivery vehicle and/or expression system for delivering the compositions, systems, or components thereof comprises liposomes, lipid particles, nanoparticles, biolistics, or viral-based expression/delivery systems.

### Considerations for Therapeutic Applications

A consideration in genome editing therapy is the choice of sequence-specific nuclease, such as a variant of a Cas nuclease. Each nuclease variant may possess its own unique set of strengths and weaknesses, many of which must be balanced in the context of treatment to maximize therapeutic benefit. For a specific editing therapy to be efficacious, a sufficiently high level of modification must be achieved in target cell populations to reverse disease symptoms. This therapeutic modification ‘threshold’ is determined by the fitness of edited cells following treatment and the amount of gene product necessary to reverse symptoms. With regard to fitness, editing creates three potential outcomes for treated cells relative to their unedited counterparts: increased, neutral, or decreased fitness. In the case of increased fitness, corrected cells may be able and expand relative to their diseased counterparts to mediate therapy. In this case, where edited cells possess a selective advantage, even low numbers of edited cells can be amplified through expansion, providing a therapeutic benefit to the patient. Where the edited cells possess no change in fitness, an increase the therapeutic modification threshold can be warranted. As such, significantly greater levels of editing may be needed to treat diseases, where editing creates a neutral fitness advantage, relative to diseases where editing creates increased fitness for target cells. If editing imposes a fitness disadvantage, as would be the case for restoring function to a tumor suppressor gene in cancer cells, modified cells would be outcompeted by their diseased counterparts, causing the benefit of treatment to be low relative to editing rates. This may be overcome with supplemental therapies to increase the potency and/or fitness of the edited cells relative to the diseased counterparts.

In addition to cell fitness, the amount of gene product necessary to treat disease can also influence the minimal level of therapeutic genome editing that can treat or prevent a disease or a symptom thereof. In cases where a small change in the gene product levels can result in significant changes in clinical outcome, the minimal level of therapeutic genome editing is less relative to cases where a larger change in the gene product levels are needed to gain a clinically relevant response. In some embodiments, the minimal level of therapeutic genome editing can range from 0.1 to 1%, 1-5%, 5-10%, 10-15%, 15-20%, 20-25%, 25-30%, 30-35%, 35-40%, 40-45%. 45-50%, or 50-55%. Thus, where a small change in gene product levels can influence clinical outcomes and diseases where there is a fitness advantage for edited cells, are ideal targets for genome editing therapy, as the therapeutic modification threshold is low enough to permit a high chance of success.

The activity of NHEJ and HDR DSB repair can vary by cell type and cell state. NHEJ is not highly regulated by the cell cycle and is efficient across cell types, allowing for high levels of gene disruption in accessible target cell populations. In contrast, HDR acts primarily during S/G2 phase, and is therefore restricted to cells that are actively dividing, limiting treatments that require precise genome modifications to mitotic cells [Ciccia, A. & Elledge, S. J. Molecular cell 40, 179-204 (2010); Chapman, J. R., et al. Molecular cell 47, 497-510 (2012)].

The efficiency of correction via HDR may be controlled by the epigenetic state or sequence of the targeted locus, or the specific repair template configuration (single vs. double stranded, long vs. short homology arms) used [Hacein-Bey-Abina, S., et al. The New England journal of medicine 346, 1185-1193 (2002); Gaspar, H. B., et al. Lancet 364, 2181-2187 (2004); Beumer, K. J., et al. G3 (2013)]. The relative activity of NHEJ and HDR machineries in target cells may also affect gene correction efficiency, as these pathways may compete to resolve DSBs [Beumer, K. J., et al. Proceedings of the National Academy of Sciences of the United States of America 105, 19821-19826 (2008)]. HDR also imposes a delivery challenge not seen with NHEJ strategies, as it uses the concurrent delivery of nucleases and repair templates. Thus, these differences can be kept in mind when designing, optimizing, and/or selecting a CRISPR-Cas based therapeutic as described in greater detail elsewhere herein.

CRISPR-Cas-based polynucleotide modification application can include combinations of proteins, small RNA molecules, and/or repair templates, and can make, in some embodiments, delivery of these multiple parts substantially more challenging than, for example, traditional small molecule therapeutics. Two main strategies for delivery of compositions, systems, and components thereof have been developed: ex vivo and in vivo. In some embodiments of ex vivo treatments, diseased cells are removed from a subject, edited and then transplanted back into the patient. In other embodiments, cells from a healthy allogeneic donor are collected, modified using a CRISPR-Cas system or component thereof, to impart various functionalities and/or reduce immunogenicity, and administered to an allogeneic recipient in need of treatment. Ex vivo editing has the advantage of allowing the target cell population to be well defined and the specific dosage of therapeutic molecules delivered to cells to be specified. The latter consideration may be particularly important when off-target modifications are a concern, as titrating the amount of nuclease may decrease such mutations (Hsu et al., 2013). Another advantage of ex vivo approaches is the typically high editing rates that can be achieved, due to the development of efficient delivery systems for proteins and nucleic acids into cells in culture for research and gene therapy applications.

In vivo polynucleotide modification via compositions, systems, and/or components thereof involves direct delivery of the compositions, systems, and/or components thereof to cell types in their native tissues. In vivo polynucleotide modification via compositions, systems, and/or components thereof allows diseases in which the affected cell population is not amenable to ex vivo manipulation to be treated. Furthermore, delivering compositions, systems, and/or components thereof to cells in situ allows for the treatment of multiple tissue and cell types.

In some embodiments, such as those where viral vector systems are used to generate viral particles to deliver the CRISPR-Cas system and/or component thereof to a cell, the total cargo size of the CRISPR-Cas system and/or component thereof should be considered as vector systems can have limits on the size of a polynucleotide that can be expressed therefrom and/or packaged into cargo inside of a viral particle. In some embodiments, the tropism of a vector system, such as a viral vector system, should be considered as it can impact the cell type to which the CRISPR-Cas system or component thereof can be efficiently and/or effectively delivered.

When delivering a system or component thereof via a viral-based system, it can be important to consider the amount of viral particles that will be needed to achieve a therapeutic effect so as to account for the potential immune response that can be elicited by the viral particles when delivered to a subject or cell(s). When delivering a system or component thereof via a viral based system, it can be important to consider mechanisms of controlling the distribution and/or dosage of the system in vivo. Generally, to reduce the potential for off-target effects, it is optimal but not necessarily required, that the amount of the system be as close to the minimum or least effective dose. In practice this can be challenging to do.

In some embodiments, it can be important to considered the immunogenicity of the system or component thereof. In embodiments, where the immunogenicity of the system or component thereof is of concern, the immunogenicity system or component thereof can be reduced. By way of example only, the immunogenicity of the system or component thereof can be reduced using the approach set out in Tangri et al. Accordingly, directed evolution or rational design may be used to reduce the immunogenicity of the CRISPR enzyme in the host species (human or other species).

### Xenotransplantation

The present invention also contemplates use of the CRISPR-Cas system described herein, e.g. Cas effector protein systems, to provide RNA-guided DNA nucleases adapted to be used to provide modified tissues for transplantation. For example, RNA-guided DNA nucleases may be used to knockout, knockdown or disrupt selected genes in an animal, such as a transgenic pig (such as the human heme oxygenase-1 transgenic pig line), for example by disrupting expression of genes that encode epitopes recognized by the human immune system, i.e. xenoantigen genes. Candidate porcine genes for disruption may for example include α(1,3)-galactosyltransferase and cytidine monophosphate-N-acetylneuraminic acid hydroxylase genes (see PCT Patent Publication WO 2014/066505). In addition, genes encoding endogenous retroviruses may be disrupted, for example the genes encoding all porcine endogenous retroviruses (see Yang et al., 2015, Genome-wide inactivation of porcine endogenous retroviruses (PERVs), Science 27 Nov. 2015: Vol. 350 no. 6264 pp. 1101-1104). In addition, RNA-guided DNA nucleases may be used to target a site for integration of additional genes in xenotransplant donor animals, such as a human CD55 gene to improve protection against hyperacute rejection.

Embodiments of the invention also relate to methods and compositions related to knocking out genes, amplifying genes and repairing particular mutations associated with DNA repeat instability and neurological disorders (Robert D. Wells, Tetsuo Ashizawa, Genetic Instabilities and Neurological Diseases, Second Edition, Academic Press, Oct. 13, 2011-Medical). Specific aspects of tandem repeat sequences have been found to be responsible for more than twenty human diseases (New insights into repeat instability: role of RNADNA hybrids. McIvor E. I., Polak U, Napierala M. RNA Biol. 2010 September-October; 7(5):551-8). The present effector protein systems may be harnessed to correct these defects of genomic instability.

Several further aspects of the invention relate to correcting defects associated with a wide range of genetic diseases which are further described on the website of the National Institutes of Health under the topic subsection Genetic Disorders (website at health.nih.gov/topic/GeneticDisorders). The genetic brain diseases may include but are not limited to Adrenoleukodystrophy, Agenesis of the Corpus Callosum, Aicardi Syndrome, Alpers' Disease, Alzheimer's Disease, Barth Syndrome, Batten Disease, CADASIL, Cerebellar Degeneration, Fabry's Disease, Gerstmann-Straussler-Scheinker Disease, Huntington's Disease and other Triplet Repeat Disorders, Leigh's Disease, Lesch-Nyhan Syndrome, Menkes Disease, Mitochondrial Myopathies and NINDS Colpocephaly. These diseases are further described on the website of the National Institutes of Health under the subsection Genetic Brain Disorders.

### Immune Orthogonal Orthologs

In some embodiments, when the Cas need to be expressed or administered in a subject, immunogenicity of the Cas may be reduced by sequentially expressing or administering immune orthogonal orthologs of the Cas to the subject. As used herein, the term “immune orthogonal orthologs” refer to orthologous proteins that have similar or substantially the same function or activity, but have no or low cross-reactivity with the immune response generated by one another. In some embodiments, sequential expression or administration of such orthologs elicits low or no secondary immune response. The immune orthogonal orthologs can avoid being neutralized by antibodies (e.g., existing antibodies in the host before the orthologs are expressed or administered). Cells expressing the orthologs can avoid being cleared by the host's immune system (e.g., by activated CTLs). In some examples, CRISPR enzyme orthologs from different species may be immune orthogonal orthologs.

Immune orthogonal orthologs may be identified by analyzing the sequences, structures, and/or immunogenicity of a set of candidates orthologs. In an example method, a set of immune orthogonal orthologs may be identified by a) comparing the sequences of a set of candidate orthologs (e.g., orthologs from different species) to identify a subset of candidates that have low or no sequence similarity; b) assessing immune overlap among the members of the subset of candidates to identify candidates that have no or low immune overlap. In some cases, immune overlap among candidates may be assessed by determining the binding (e.g., affinity) between a candidate ortholog and MHC (e.g., MHC type I and/or MHC II) of the host. Alternatively or additionally, immune overlap among candidates may be assessed by determining B-cell epitopes for the candidate orthologs. In one example, immune orthogonal orthologs may be identified using the method described in Moreno A M et al., BioRxiv, published online Jan. 10, 2018, doi: doi.org/10.1101/245985.

### Patient-Specific Screening Methods

A nucleic acid-targeting system that targets RNA or single stranded DNA can be used to screen patients or patient samples for the presence of particular RNA or single stranded DNA. Methods may comprise detection of one or more viruses in a sample from the patient. Advantageously, rapid detection using one or more CRISPR Cas systems can identify those patients with particular viral infections.

### Transcript Detection Methods

The effector proteins and systems of the invention are useful for specific detection of RNAs in a cell or other sample. In the presence of an RNA target of interest, guide-dependent CRISPR-Cas nuclease activity may be accompanied by non-specific RNAse activity against collateral targets. To take advantage of the RNase activity, all that is needed is a reporter substrate that can be detectably cleaved. For example, a reporter molecule can comprise RNA, tagged with a fluorescent reporter molecule (fluor) on one end and a quencher on the other. In the absence of CRISPR-Cas RNase activity, the physical proximity of the quencher dampens fluorescence from the fluor to low levels. When CRISPR-Cas target specific cleavage is activated by the presence of an RNA target-of-interest and suitable guide RNA, the RNA-containing reporter molecule is non-specifically cleaved and the fluor and quencher are spatially separated. This causes the fluor to emit a detectable signal when excited by light of the appropriate wavelength. In one exemplary assay method, CRISPR-Cas effector, target-of-interest-specific guide RNA, and reporter molecule are added to a cellular sample. An increase in fluorescence indicates the presence of the RNA target-of-interest. In another exemplary method, a detection array is provided. Each location of the array is provided with CRISPR-Cas effector, reporter molecule, and a target-of-interest-specific guide RNA. Depending on the assay to be performed, the target-of-interest-specific guide RNAs at each location of the array can be the same, different, or a combination thereof. Different target-of-interest-specific guide RNAs might be provided, for example when it is desired to test for one or more targets in a single source sample. The same target-of-interest-specific guide RNA might be provided at each location, for example when it is desired to test multiple samples for the same target.

In certain embodiments, CRISPR-Cas is provided or expressed in an in vitro system or in a cell, transiently or stably, and targeted or triggered to non-specifically cleave cellular nucleic acids. In one embodiment, CRISPR-Cas is engineered to knock down ssDNA, for example viral ssDNA. In another embodiment, CRISPR-Cas is engineered to knock down RNA. The system can be devised such that the knockdown is dependent on a target DNA present in the cell or in vitro system, or triggered by the addition of a target nucleic acid to the system or cell.

In an embodiment, the CRISPR-Cas system is engineered to non-specifically cleave RNA in a subset of cells distinguishable by the presence of an aberrant DNA sequence, for instance where cleavage of the aberrant DNA might be incomplete or ineffectual. In one non-limiting example, a DNA translocation that is present in a cancer cell and drives cell transformation is targeted. Whereas a subpopulation of cells that undergoes chromosomal DNA and repair may survive, non-specific collateral ribonuclease activity advantageously leads to cell death of potential survivors.

### Kits

In one aspect, the invention provides kits containing any one or more of the elements disclosed in the above methods and compositions. In some embodiments, the kit comprises a vector system as taught herein or one or more of the components of the CRISPR/Cas system or complex as taught herein, such as crRNAs and/or CRISPR-Cas effector protein or CRISPR-Cas effector protein encoding mRNA, and instructions for using the kit. Elements may be provided individually or in combinations, and may be provided in any suitable container, such as a vial, a bottle, or a tube. In some embodiments, the kit includes instructions in one or more languages, for example in more than one language. The instructions may be specific to the applications and methods described herein. In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g., in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide or crRNA sequence and a regulatory element. In some embodiments, the kit comprises a homologous recombination template polynucleotide. In some embodiments, the kit comprises one or more of the vectors and/or one or more of the polynucleotides described herein. The kit may advantageously allow to provide all elements of the systems of the invention.

The present application also provides aspects and embodiments as set forth in the following numbered Statements:
Statement 1. A non-naturally occurring or engineered composition comprising:
   (a) a Cas protein that comprises at least one HEPN domain and is less than 900 amino acids in size; and (b) a guide sequence capable of forming of complex with the Cas protein and directing the complex to bind to a target sequence.
Statement 2. The composition of Statement 1, wherein the Cas protein is a Type VI Cas protein.
Statement 3. The composition of Statement 1, wherein the Cas protein is Cas13.
Statement 4. The composition of Statement 1, wherein the Cas protein is selected from (a) SEQ ID NOs. 4102-4298; (b) SEQ ID NOs. 4299-4654; (c) SEQ ID NOs. 2771-2772, 4655-4768, or 5260-5265; (d) SEQ ID NOs. 4769-4797; or (e) SEQ ID NOs. 4798-5203.
Statement 5. A non-naturally occurring or engineered system comprising: (a) a Cas protein selected from: (i) SEQ ID NOs. 1-1323, (ii) SEQ ID NOs. 1324-2770, (iii) SEQ ID NOs. 2773-2797, or (iv) SEQ ID NOs. 2798-4092; (b) a guide sequence capable of forming of complex with the Cas protein and directing the complex to bind to a target sequence.
Statement 6. The composition of any one of the proceeding Statements, wherein the Cas protein exhibits collateral nuclease activity and cleaves a non-target sequence.
Statement 7. The composition of any one of the proceeding Statements, which comprises two or more guide sequences capable of hybridizing to two different target sequences or different regions of a target sequence.
Statement 8. The composition of any one of the proceeding Statements, wherein the guide sequence is capable of hybridizing to one or more target sequences in a prokaryotic cell.
Statement 9. The composition of any one of the proceeding Statements, wherein the guide sequence is capable of hybridizing to one or more target sequences in a eukaryotic cell.
Statement 10. The composition of any one of the proceeding Statements, wherein the Cas protein comprises one or more nuclear localization signals.
Statement 11. The composition of any one of the proceeding Statements, wherein the Cas protein comprises one or more nuclear export signals.
Statement 12. The composition of any one of the proceeding Statements, wherein the Cas protein is catalytically inactive.
Statement 13. The composition of any one of the proceeding Statements, wherein the Cas protein is a nickase.
Statement 14. The composition of any one of the proceeding Statements, wherein the Cas protein is associated with one or more functional domains.
Statement 15. The composition of Statement 14, wherein the one or more functional domains is heterologous functional domains.
Statement 16. The composition of Statement 14, wherein the one or more functional domains cleaves the one or more target sequences.
Statement 17. The composition of Statement 16, wherein the one or more functional domains modifies transcription or translation of the target sequence.
Statement 18. The composition of any one of the proceeding Statements, wherein the Cas protein is associated with an adenosine deaminase or cytidine deaminase.
Statement 19. The composition of any one of the proceeding Statements, further comprising a recombination template.
Statement 20. The composition of Statement 19, wherein the recombination template is inserted by homology-directed repair (HDR).
Statement 21. The composition of any one of the proceeding Statements, further comprising a tracr RNA.
Statement 22. The composition of any one of the proceeding Statements, wherein the Cas protein comprises two HEPN domains.
Statement 23. A non-naturally occurring or engineered composition comprising: (a) an mRNA encoding the Cas protein of any one of the proceeding Statements, and (b) a guide sequence capable of forming of complex with the Cas protein and directing the complex to bind to a target sequence.
Statement 24. A non-naturally occurring or engineered composition for modifying nucleotides in a target nucleic acid, comprising: (a) the composition of any one of Statements 1-22; and (b) a nucleotide deaminase associated with the Cas protein.
Statement 25. The composition of Statement 24, wherein the Cas protein is a dead Cas protein.
Statement 26. The composition of any one of Statements 24-25, wherein the Cas protein is a nickase.
Statement 27. The composition of any one of Statements 24-26, wherein the nucleotide deaminase is covalently or non-covalently linked to the Cas protein or the guide sequence, or is adapted to link thereof after delivery.
Statement 28. The composition of any one of Statements 24-27, wherein the nucleotide deaminase is a adenosine deaminase.
Statement 29. The composition of any one of Statements 24-28, wherein the nucleotide deaminase is a cytidine deaminase.
Statement 30. The composition of any one of Statements 24-29, wherein the nucleotide deaminase is a human ADAR2 or a deaminase domain thereof.
Statement 31. The composition of Statement 28, wherein the adenosine deaminase comprises one or more mutations.
Statement 32. The composition of Statement 31, wherein the one or more mutations comprise E620G or Q696L based on amino acid sequence positions of human ADAR2, and corresponding mutations in a homologous ADAR protein.
Statement 33. The composition of Statement 32, wherein the adenosine deaminase comprises (i) E488Q and E620G, (ii) E488Q and Q696L, or (iii) E488Q and V505I, based on amino acid sequence positions of human ADAR2, or corresponding mutations in a homologous ADAR protein.
Statement 34. The composition of Statement 31, wherein the adenosine deaminase has cytidine deaminase activity.
Statement 35. The composition of any one of Statements 24-34, wherein the nucleotide deaminase protein or catalytic domain thereof has been modified to increase activity against a DNA-RNA heteroduplex.
Statement 36. The composition of any one of Statements 24-35, wherein the nucleotide deaminase protein or catalytic domain thereof has been modified to reduce off-target effects.
Statement 37. The composition of any one of Statements 24-36, wherein modification of the nucleotides in the target nucleic acid remedies a disease caused by a G→A or C-T point mutation or a pathogenic SNP.
Statement 38. The composition of Statement 37, wherein the disease comprises cancer, haemophilia, beta-thalassemia, Marfan syndrome, and Wiskott-Aldrich syndrome.
Statement 39. The composition of any one of Statements 24-38, wherein modification of the nucleotides in the target nucleic acid remedies a disease caused by a T-C or A-G point mutation or a pathogenic SNP.
Statement 40. The composition of any one of Statements 24-39, wherein modification of the nucleotide at the target locus of interest inactivates a target gene at the target locus.
Statement 41. The composition of any one of Statements 24-40, wherein modification of the nucleotide modifies gene product encoded at the target locus or expression of the gene product.
Statement 42. An engineered adenosine deaminase comprising one or more mutations: E488Q, E620G, Q696L, or V505I based on amino acid sequence positions of human ADAR2, or corresponding mutations in a homologous ADAR protein.
Statement 43. The engineered adenosine deaminase of Statement 42, wherein the adenosine deaminase comprises (i) E488Q and E620G, (ii) E488Q and Q696L, or (iii) E488Q and V505I based on amino acid sequence positions of human ADAR2, or corresponding mutations in a homologous ADAR protein.
Statement 44. A system for detecting presence of one or more target polypeptides in one or more in vitro samples comprising: a Cas protein of any one of Statements 1 to 41; one or more detection aptamers, each designed to bind to one of the one or more target polypeptides, each detection aptamer comprising a masked promoter binding site or masked primer binding site and a trigger sequence template; and an oligonucleotide-based masking construct comprising a non-target sequence.
Statement 45. The system of Statement 44, further comprising nucleic acid amplification reagents to amplify the target sequence or the trigger sequence.
Statement 46. The system of Statement 45, wherein the nucleic acid amplification reagents are isothermal amplification reagents.
Statement 47. A system for detecting the presence of one or more target sequences in one or more in vitro samples, comprising: (a) a Cas protein of any one of Statements 1 to 41; (b) at least one guide polynucleotide comprising a guide sequence designed to have a degree of complementarity with the one or more target sequences, and designed to form a complex with the Cas protein; and (c) an oligonucleotide-based masking construct comprising a non-target sequence, wherein the Cas protein exhibits collateral nuclease activity and cleaves the non-target sequence of the oligo-nucleotide based masking construct once activated by the one or more target sequences.
Statement 48. A non-naturally occurring or engineered composition comprising the Cas protein of any one of Statements 1 to 41 that is linked to an inactive first portion of an enzyme or reporter moiety, wherein the enzyme or reporter moiety is reconstituted when contacted with a complementary portion of the enzyme or reporter moiety.
Statement 49. The composition of Statement 48, wherein the enzyme or reporter moiety comprises a proteolytic enzyme.
Statement 50. The composition of Statement 48 or 49, wherein the Cas protein comprises a first Cas protein and a second Cas protein linked to the complementary portion of the enzyme or reporter moiety.
Statement 51. The composition of Statement 48, 49, or 50, further comprising: i) a first guide capable of forming a complex with the first Cas protein and hybridizing to a first target sequence of a target nucleic acid; and ii) a second guide capable of forming a complex with the second Cas protein, and hybridizing to a second target sequence of the target nucleic acid.
Statement 52. A non-naturally occurring or engineered composition comprising one or more polynucleotides encoding the Cas protein and the guide sequence in any one of Statements 1 to 41.
Statement 53. A vector system, which comprises one or more vectors comprising: a first regulatory element operably linked to a nucleotide sequence encoding a Cas protein of any one of Statements 1 to 41, and a second regulatory element operably linked to a nucleotide sequence encoding the guide sequence.
Statement 54. The vector system of Statement 53, wherein the nucleotide sequence encoding the Cas protein is codon optimized for expression in a eukaryotic cell.
Statement 55. The vector system of Statement 53 or 54, which is comprised in a single vector.
Statement 56. The vector system of any one of Statements 53-55, wherein the one or more vectors comprise viral vectors.
Statement 57. The vector system of any one of Statements 53-56, wherein the one or more vectors comprise one or more retroviral, lentiviral, adenoviral, adeno-associated or herpes simplex viral vectors.
Statement 58. A delivery system comprising the composition of any one of Statements 1 to 52, or the system of any one of Statements 53 to 57 and a delivery vehicle.
Statement 59. The delivery system of Statement 58, which comprises one or more vectors, or one or more polynucleotide molecules, the one or more vectors or polynucleotide molecules comprising one or more polynucleotide molecules encoding the Cas protein and one or more nucleic acid components of the non-naturally occurring or engineered composition.
Statement 60. The delivery system of Statement 58, wherein the delivery vehicle comprises a ribonucleoprotein complex, one or more particles, one or more vesicles, or one or more viral vectors, liposomes, nanoparticles, exosomes, microvesicles, nucleic acid nanoassemblies, a gene gun, an implantable device, or a vector system.
Statement 61. The delivery system of Statement 58, wherein the one or more particles comprises a lipid, a sugar, a metal or a protein.
Statement 62. The delivery system of Statement 58, wherein the one or more particles comprises lipid nanoparticles.
Statement 63. The delivery system of Statement 58, wherein the one or more vesicles comprises exosomes or liposomes.
Statement 64. The delivery system of Statement 58, wherein the one or more viral vectors comprises one or more adenoviral vectors, one or more lentiviral vectors, or one or more adeno-associated viral vectors.
Statement 65. A cell comprising the composition of any one of Statements 1 to 52, or the system of any one of Statements 53 to 64.
Statement 66. The cell of Statement 65 or progeny thereof is a eukaryotic cell, preferably a human or non-human animal cell, optionally a therapeutic T cell or antibody-producing B-cell or wherein the cell is a plant cell.
Statement 67. A non-human animal or plant comprising the cell of Statement 65 or 66, or progeny thereof.
Statement 68. The composition of any one of Statements 1 to 52, or the system of any one of Statements 53 to 64, or the cell of Statement 65 or 66, for use in a therapeutic method of treatment.
Statement 69. A method of modifying one or more target sequences, the method comprising contacting the one or more target sequences with the composition of any one of Statements 1 to 52.
Statement 70. The method of Statement 69, wherein modifying the one or more target sequences comprises increasing or decreasing expression of the one or more target sequences.
Statement 71. The method of Statement 69 or 70, wherein the system further comprises a recombination template, and wherein modifying the one or more target sequences comprises insertion of the recombination template or a portion thereof.
Statement 72. The method of any one of Statements 69-71, wherein the one or more target sequences is in a prokaryotic cell.
Statement 73. The method of any one of Statements 69-72, wherein the one or more target sequences is in a eukaryotic cell.
Statement 74. A method of modifying one or more nucleotides in a target sequence, comprising contacting the target sequences with the composition of any one of any one of Statements 1 to 52.
Statement 75. The method of any one of any one of Statements 69-74, wherein the target sequence is RNA.
Statement 76. A method for detecting a target nucleic acid in a sample comprising: (a) contacting a sample with: (i) the composition of any one of Statements 1 to 52; and (ii) a RNA-based masking construct comprising a non-target sequence; wherein the Cas protein exhibits collateral RNase activity and cleaves the non-target sequence of the detection construct; and (b) detecting a signal from cleavage of the non-target sequence, thereby detecting the target nucleic acid in the sample.
Statement 77. The method of Statement 76, further comprising contacting the sample with reagents for amplifying the target nucleic acid.
Statement 78. The method of Statement 76 or 77, wherein the reagents for amplifying comprises isothermal amplification reaction reagents.
Statement 79. The method of any one of Statements 76-78, wherein the isothermal amplification reagents comprise nucleic-acid sequence-based amplification, recombinase polymerase amplification, loop-mediated isothermal amplification, strand displacement amplification, helicase-dependent amplification, or nicking enzyme amplification reagents.
Statement 80. The method of any one of Statements 76-79, wherein the target nucleic acid is DNA molecule and the method further comprises contacting the target DNA molecule with a primer comprising an RNA polymerase site and RNA polymerase.
Statement 81. The method of any one of Statements 76-80, wherein the masking construct: suppresses generation of a detectable positive signal until the masking construct cleaved or deactivated, or masks a detectable positive signal or generates a detectable negative signal until the masking construct cleaved or deactivated.
Statement 82. The method of any one of Statements 76-81, wherein the masking construct comprises: a. a silencing RNA that suppresses generation of a gene product encoded by a reporting construct, wherein the gene product generates the detectable positive signal when expressed; b. a ribozyme that generates the negative detectable signal, and wherein the positive detectable signal is generated when the ribozyme is deactivated; c. a ribozyme that converts a substrate to a first color and wherein the substrate converts to a second color when the ribozyme is deactivated; d. an aptamer and/or comprises a polynucleotide-tethered inhibitor; e. a polynucleotide to which a detectable ligand and a masking component are attached; f. a nanoparticle held in aggregate by bridge molecules, wherein at least a portion of the bridge molecules comprises a polynucleotide, and wherein the solution undergoes a color shift when the nanoparticle is disbursed in solution; g. a quantum dot or fluorophore linked to one or more quencher molecules by a linking molecule, wherein at least a portion of the linking molecule comprises a polynucleotide; h. a polynucleotide in complex with an intercalating agent, wherein the intercalating agent changes absorbance upon cleavage of the polynucleotide; or 1. two fluorophores tethered by a polynucleotide that undergo a shift in fluorescence when released from the polynucleotide.
Statement 83. The method of Statement 82, wherein the aptamer: a. comprises a polynucleotide-tethered inhibitor that sequesters an enzyme, wherein the enzyme generates a detectable signal upon release from the aptamer or polynucleotide-tethered inhibitor by acting upon a substrate; b. is an inhibitory aptamer that inhibits an enzyme and prevents the enzyme from catalyzing generation of a detectable signal from a substrate or wherein the polynucleotide-tethered inhibitor inhibits an enzyme and prevents the enzyme from catalyzing generation of a detectable signal from a substrate; or c. sequesters a pair of agents that when released from the aptamers combine to generate a detectable signal.
Statement 84. The method of Statement 82 or 83, wherein the nanoparticle is a colloidal metal.
Statement 85. The method of any one of Statements 76-84, wherein the at least one guide polynucleotide comprises a mismatch.
Statement 86. The method of Statement 85, wherein the mismatch is upstream or downstream of a single nucleotide variation on the one or more guide sequences.
Statement 87. A method of treating or preventing a disease in a subject, comprising administering the composition of any one of Statements 1 to 52, or the system of any one of Statements 53 to 64, or the cell of Statement 65 or 66 to the subject.

### EXAMPLES

### Example 1

Systems, compositions, and methods can be designed for the detection and diagnosis of viruses and viral infections, including acute respiratory infections using the disclosure detailed herein. The systems can comprise two or more CRISPR Cas systems to multiplex, as described elsewhere herein, to detect a plurality of respiratory infections or viral infections, including coronavirus. The coronavirus is a positive-sense single stranded RNA family of viruses, infecting a variety of animals and humans. SARS-CoV is one type of coronavirus infection, as well as MERS-CoV Detection of one or more coronaviruses are envisioned, including the 2019-nCoV detected in Wuhan City. Sequences of the 2019-nCoV are available at GISAID accession no. EPI_ISL_402124 and EPI_ISL_402127-402130, and described in DOI: 10.1101/2020.01.22.914952. Further deposits of the Wuhan coronavirus deposited in the GISAID platform include EP_ISL_402119-402121 and EP_ISL_402123-402124; see also GenBank Accession No. MN908947.3.

### Multiplexed Detection

Multiplex design of guide molecules for the detection of coronaviruses and/or other respiratory viruses in a sample to identify the cause of a respiratory infection is envisioned, with design can be according to the disclosure of U.S. Provisional Application 62/818,702, filed Mar. 14, 2019 and entitled “CRISPR Effector System Based Multiplex Diagnostics”, and U.S. Provisional Application 62/890,5556, filed Aug. 22, 2019, entitled “CRISPR Effector System Based Multiplex Diagnostics” incorporated herein in their entirety. Briefly, the design of guide molecules can encompass utilization of training models as described in U.S. Provisional Application 62/818,702 and U.S. Provisional Application 62/890,5556 using a variety of input features, which may include the particular Cas protein used for targeting of the sequences of interest. See U.S. Provisional Application 62/818,702 FIG. 4A, incorporated specifically by reference. Guide molecules can be designed as detailed elsewhere herein. Regarding detection of coronavirus, guide design can be predicated on genome sequences disclosed in Tian et al, “Potent binding of 2019 novel coronavirus spike protein by a SARS coronavirus-specific human monoclonal antibody”; doi: 10.1101/2020.01.28.923011, incorporated by reference, which details human monoclonal antibody, CR3022 binding of the 2019-nCoV RBD (KD of 6.3 nM) or Sequences of the 2019-nCoV are available at GISAID accession no. EPI_ISL_402124 and EPI_ISL_402127-402130, and described in doi:10.1101/2020.01.22.914952, or EP_ISL_402119-402121 and EP_ISL_402123-402124; see also GenBank Accession No. MN908947.3. Guide design can target unique viral genomic regions of the 2019-nCoV or conserved genomic regions across one or more viruses of the coronavirus family.

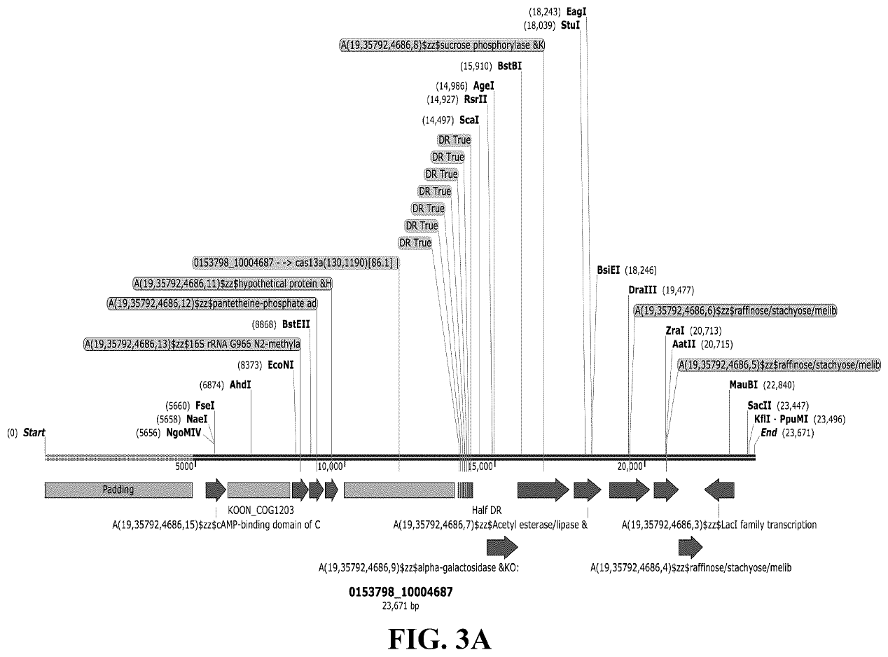

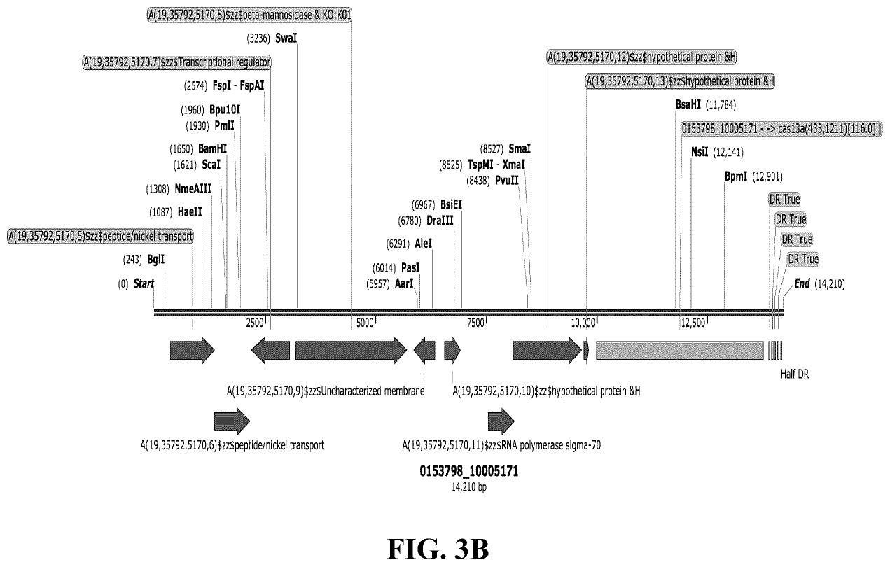

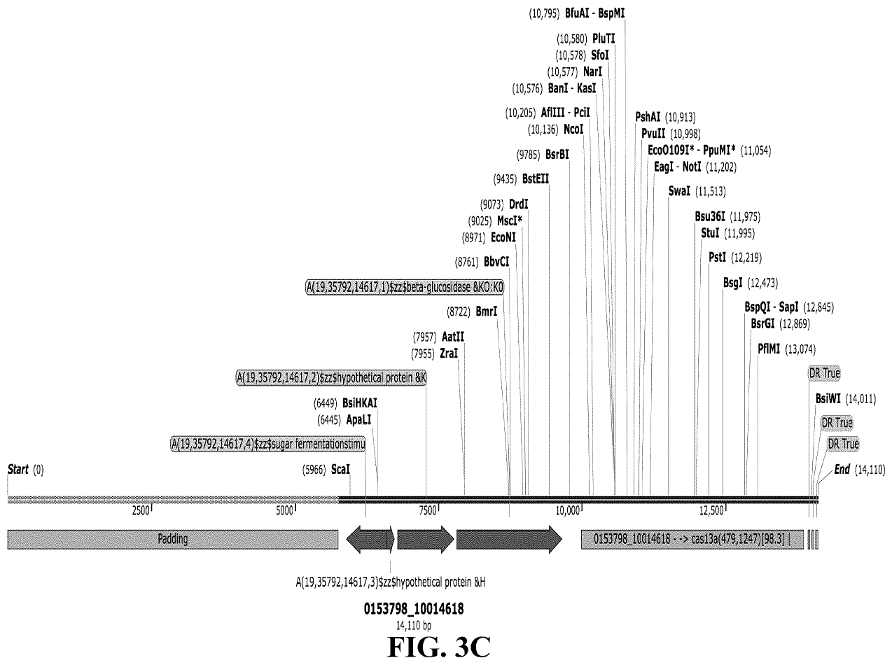

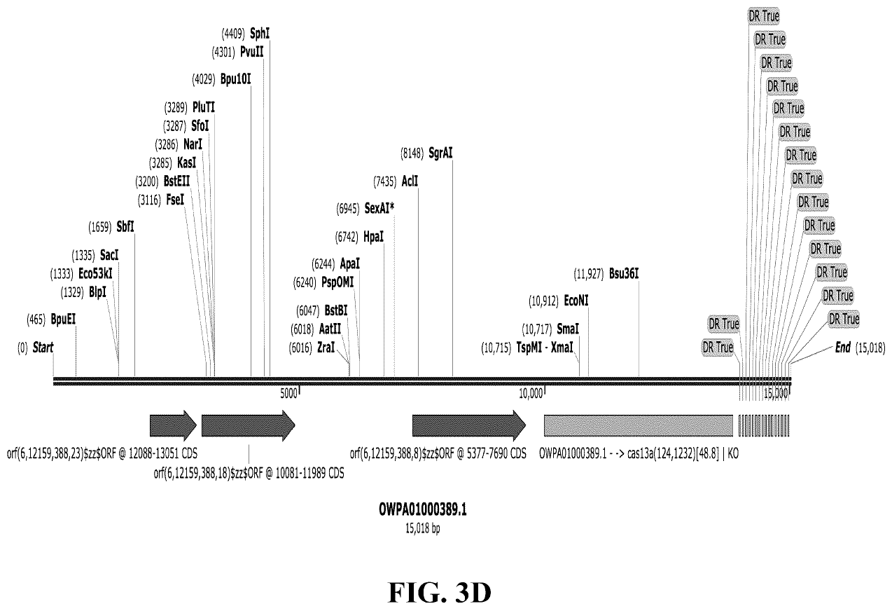

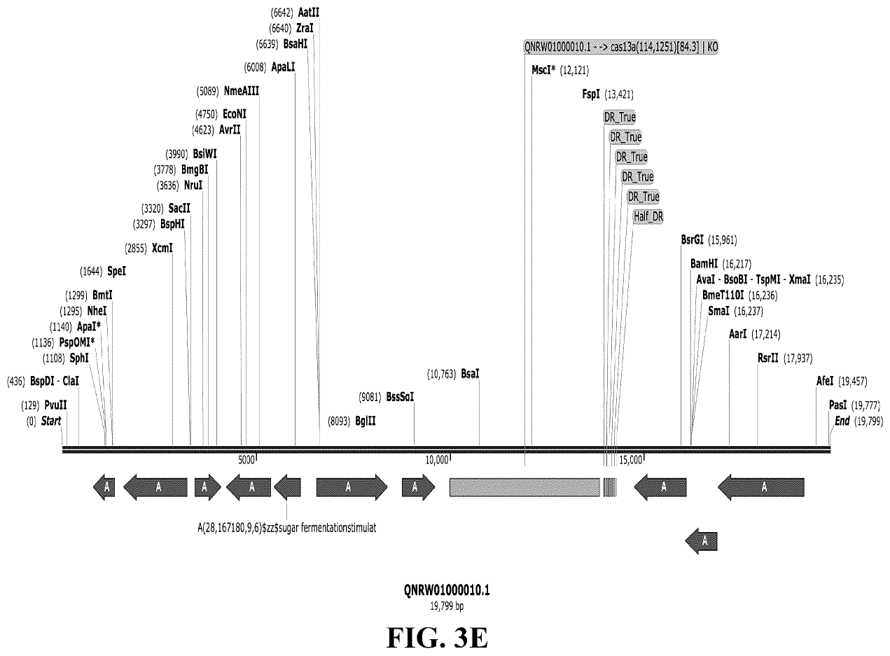

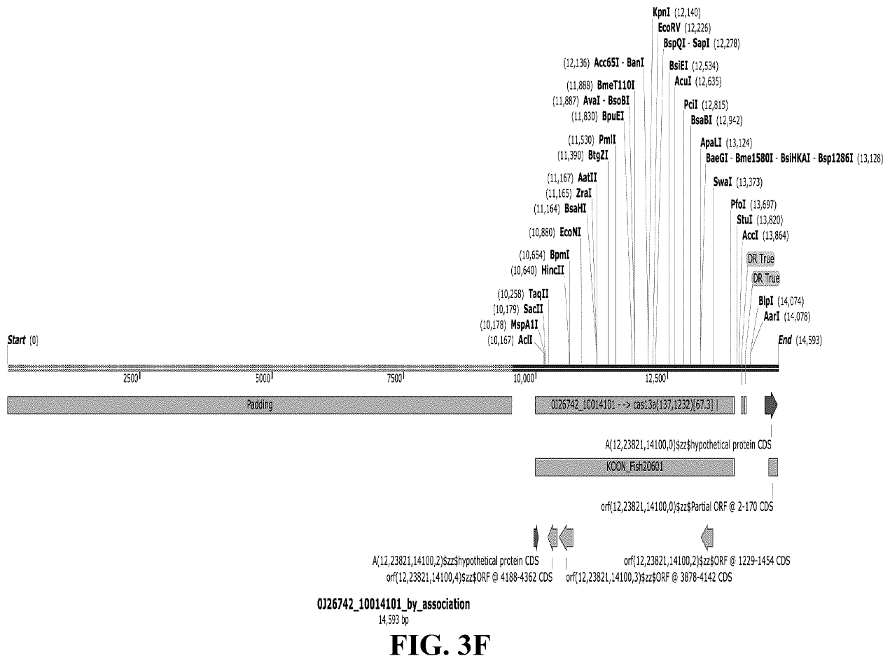

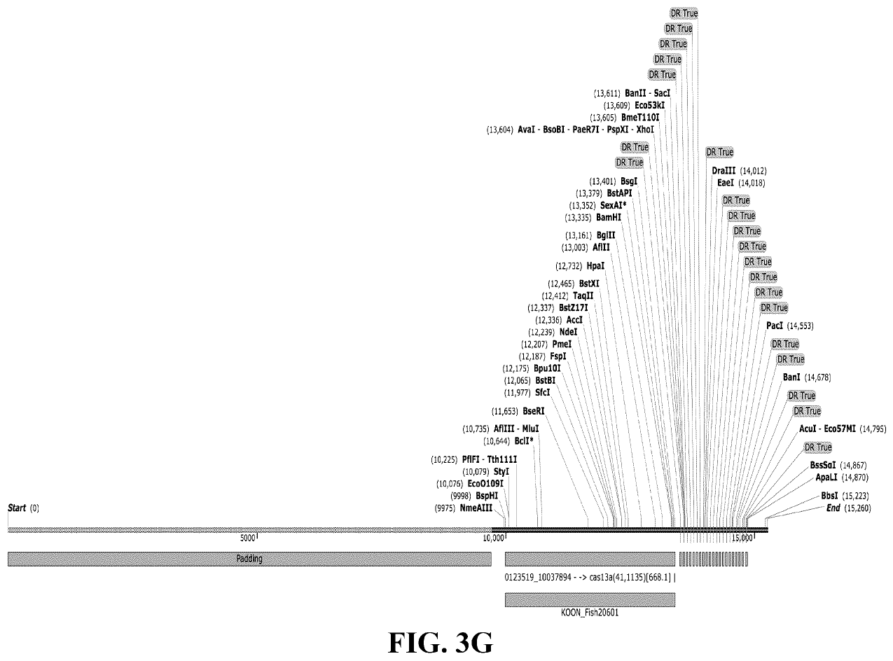

Detection of respiratory viruses such as coronavirus may include a thermostable CRISPR-Cas protein as described herein, which may be a Cas13a ortholog. As described elsewhere herein, one or more Cas13a orthologs may be utilized in a multiplex design, including the thermostable Cas13a orthologs described herein, including those derived from Herbinix hemicellulosilytica, defined by SEQ ID NO: 1, or by SEQ ID NO: 75 of International Publication No. WO 2017/219027, defined by a sequence from FIG. 1A (loci QNRW01000010.1, OWPA01000389.1, 0153798_10014618, 0153978_10005171, and 0153798_10004687), or encoded by the nucleic acid sequence 0123519_10037894 or 0J26742_10014101, or have at least 80%, 85%, 90%, 91%, 92%, 93%, 94%, 95%, 96%, 97%, 98%, 99%, or 99.5% sequence identity to SEQ ID NO: 1 or to SEQ ID NO: 75 of International Publication No. WO 2017/219027, defined by a sequence from FIG. 1A (loci QNRW01000010.1, OWPA01000389.1, 0153798_10014618, 0153978_10005171, and 0153798_10004687), or encoded by the nucleic acid sequence 0123519_10037894 (FIG. 3G) or 0J26742_10014101(FIG. 3F) and may comprise least one HEPN domain or at least two HEPN domains. Additionally, in certain example embodiments, such thermostability confers further rapidity to the diagnostic and detections platforms and methods disclosed herein.

Coronavirus detection can comprise two or more detection systems utilizing RNA targeting Cas effector proteins; DNA targeting Cas effector proteins, or a combination thereof. The RNA-targeting effector proteins may be a Cas13 protein, such as Cas13a, Cas13b, or Cas13c, including one of the thermostable Cas13a proteins described herein. Multiplexing systems can be designed such that different Cas proteins with different sequence specificities or other motif cutting preferences can be used, including, in certain embodiments, at least one Cas13a thermostable protein described herein. See International Publication WO 2019/126577. Type VI and Type V Cas proteins are known to possess different cutting motif preferences. See Gootenberg et al. “Multiplexed and portable nucleic acid detection platform with Cas13b, Cas12a, and Csm6.” Science. Apr. 27, 2018, 360:439-444; International Publication WO 2019/051318. Thus, embodiments disclosed herein may further comprise multiplex embodiments comprising two or more Type VI Cas proteins with different cutting preferences, or one or more Type VI Cas proteins and one or more Type V case proteins.

Multiplex approaches and selection of Cas effector proteins can be as described in International Publication WO 2019/126577 at [0415]—[0416] and Examples 1-10, incorporated herein by reference. In certain example embodiments, the coronavirus assay comprises a Type VI Cas protein disclosed herein and guide molecule comprising a guide sequence configured to directed binding of the CRISPR-Cas complex to a target molecule and a labeled detection molecule (“RNA-based masking construct”). A multiplex embodiment can be designed to track one or more variants of coronavirus or one or more variants of coronavirus, including the 2019-nCoV, in combination with other viruses, for example, Human respiratory syncytial virus, Middle East respiratory syndrome (MERS) coronavirus, Severe acute respiratory syndrome-related (SARS) coronavirus, and influenza.

In certain embodiments, the detection assay can be provided on a lateral flow device, as described in International Publication WO 2019/071051, incorporated herein by reference. The lateral flow device can be adapted to detect one or more coronaviruses and/or other viruses in combination of the coronavirus. The lateral flow device may comprise a flexible substrate, such as a paper substrate or a flexible polymer-based substrate, which can include freeze-dried reagents for detection assays with a visual readout of the assay results. See, WO 2019/071051 at [0145]-[0151] and Example 2, specifically incorporated herein by reference. In certain embodiments, the coronavirus assay can be utilized with isothermal amplification reagents, allowing amplification without complex instrumentation that may be unavailable in the field as described in WO 2019/071051. Accordingly, the assay can be adapted for field diagnostics, including use of visual readout on a lateral flow device, rapid, sensitive detection and can be deployed for early and direct detection.

### Example 2—Identification and Characterization of Novel Crispr-Cas Systems

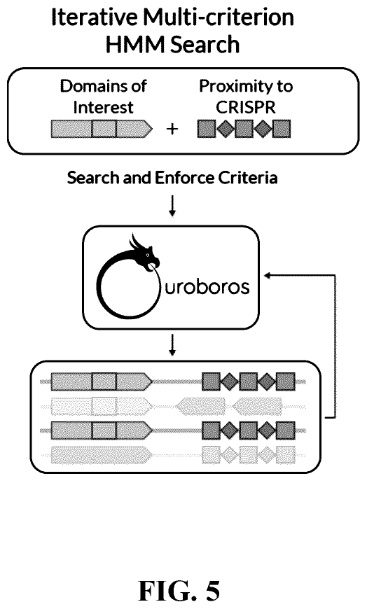

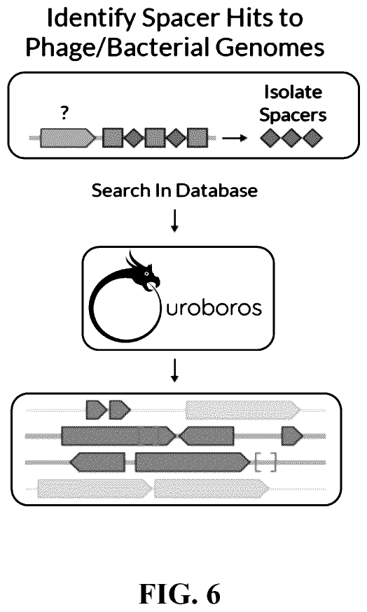

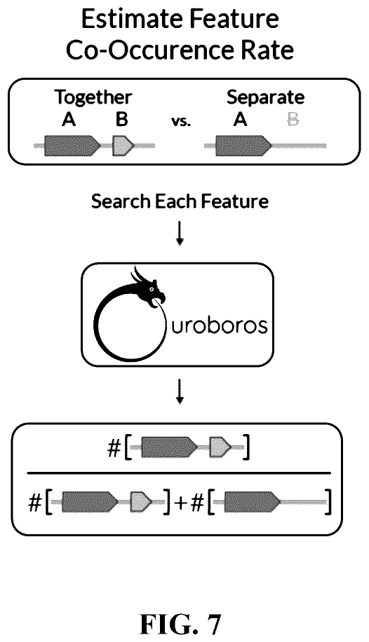

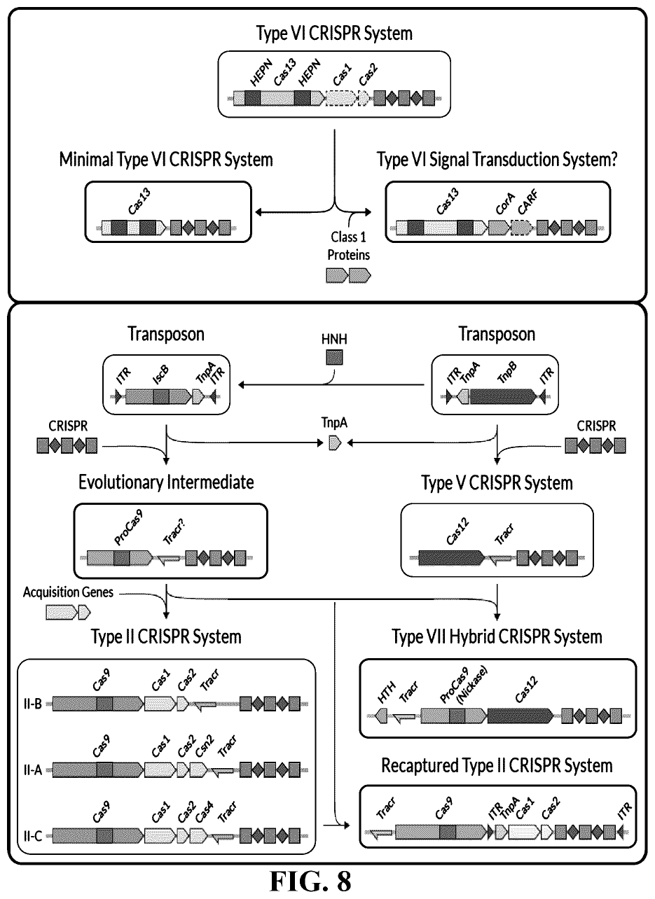

To identify novel Cas proteins, proteins operonized with Cas1/Cas2, large proteins near CRISPRs, and proteins co-evolving with known Cas Genes were identified using (FIG. 4). Iterative multi-criterion HMM searches were conducted (FIG. 5). Spacer hits to phage/bacterial genomes were identified (FIG. 6). Estimated feature co-occurrence rates were determined (FIG. 7). FIG. 8 shows hypothesized evolution of various CRISPR systems.

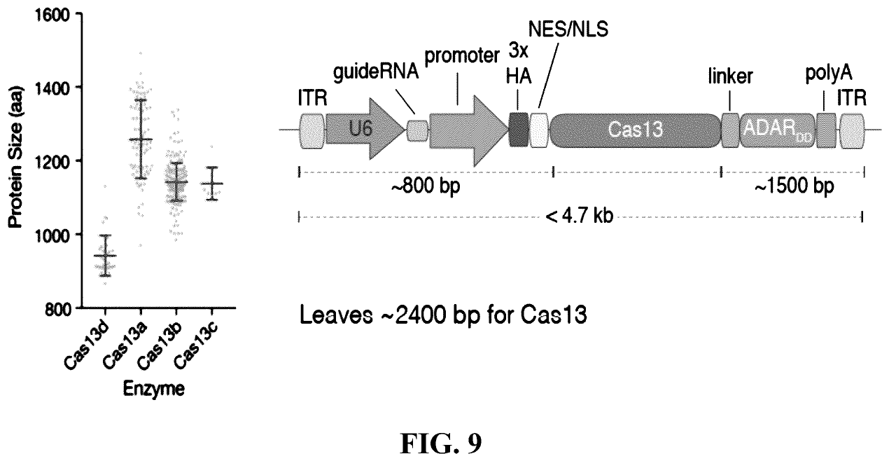

The distribution of sizes of proteins in Cas13 families are shown in FIG. 9.

In exemplary Cas13b-ts, small RNA sequencing of the locus in E. coli shows that the only associated small RNA is the CRISPR RNA. 3 exemplary Cas13b-ts were found active in E. coli in an essential gene knockdown screen. This screen also allowed for the determination of the protospacer flanking motif (PFS) of the active orthologs, which was found to be a weak 5′ not C preference for all active orthologs. In mammalian cells, the three active orthologs were also found to be active for mediating knockdown of both luciferase reporter and endogenous transcripts. Fused to ADAR, Cas13b-t1 and t3 were able to mediate A-to-I RNA editing of both reporter and endogenous transcripts in multiple cell types. Additionally, fusion to a directedly-evolved CDAR mediated C-to-U RNA editing of reporter transcripts. Small proteins that can mediate nucleic acid modification were useful for delivery of gene therapy agents via adeno-associated virus (AAV). Current plans included delivery of a Cas13b-t1-ADAR fusion via AAV as well as a guide RNA expression cassette targeting the beta catenin transcript in mice to demonstrate effective Cas13-mediated RNA editing in an in vivo setting via AAV delivery.

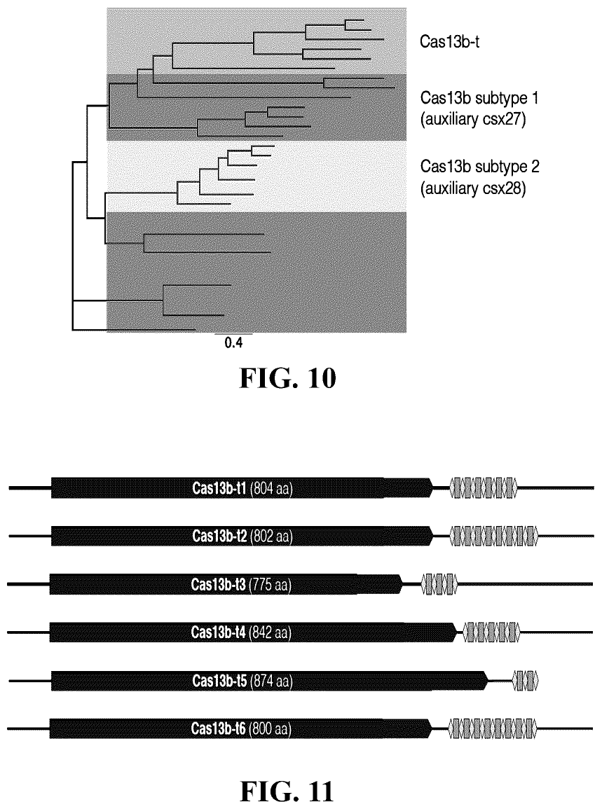

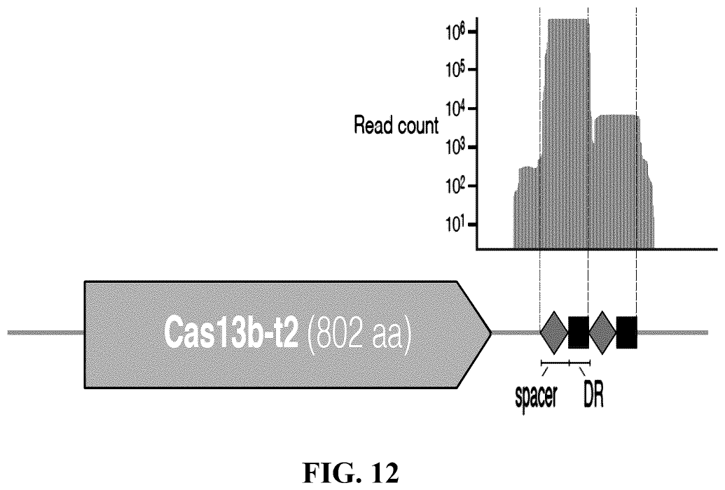

Cas13b-t proteins were identified from the analysis. The Cas13b-t proteins are a subgroup of Type VI-B1 with no auxiliary proteins (FIG. 10). 6 examples of Cas13b-ts are shown in FIG. 11. The analysis suggested that CRISPR arrays in Cas13b-t loci were processed and no other ncRNAs were present (FIG. 12).

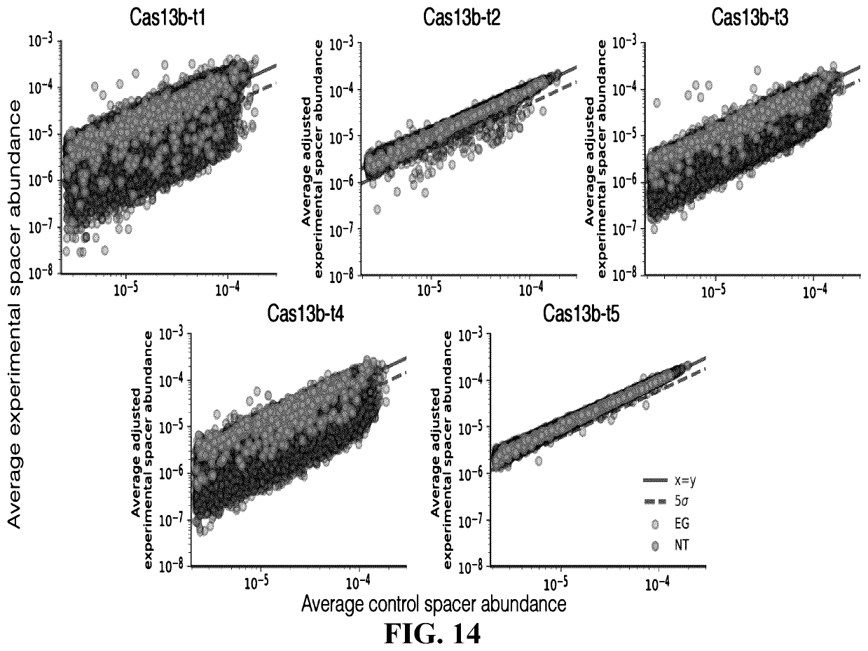

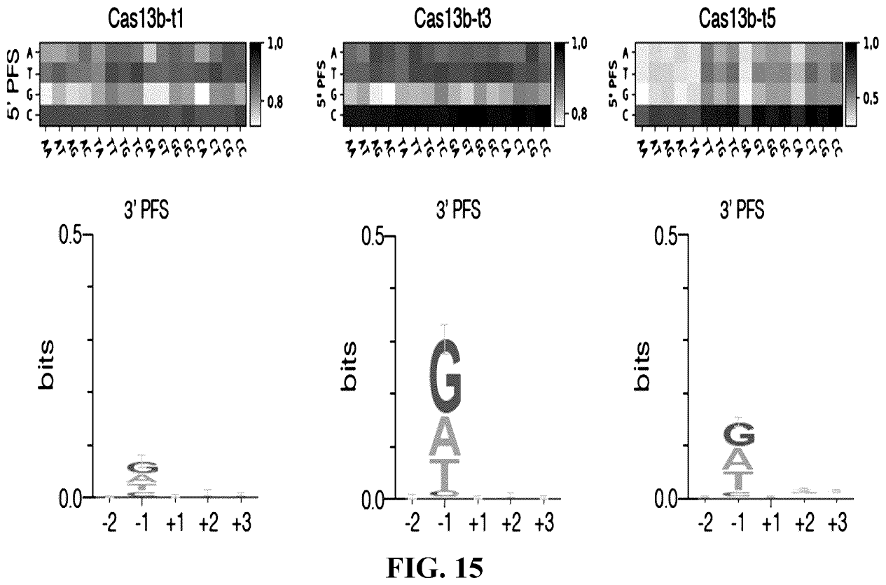

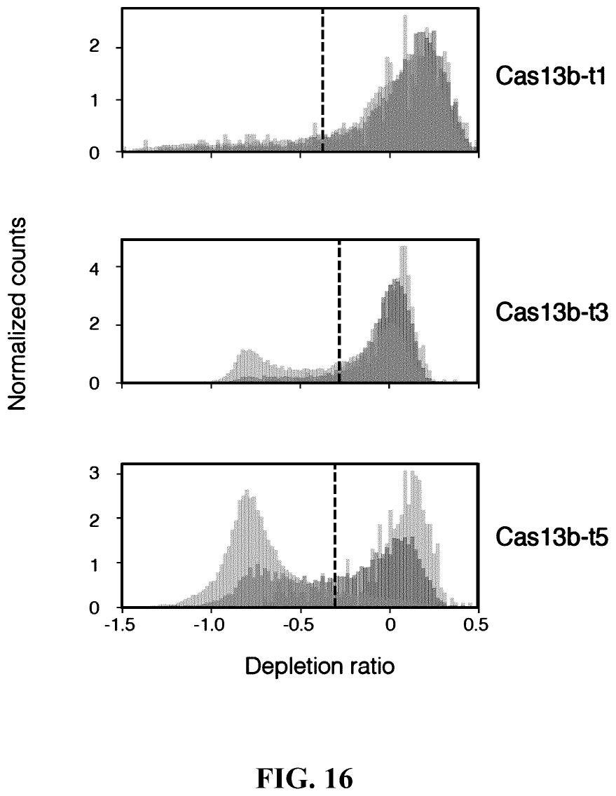

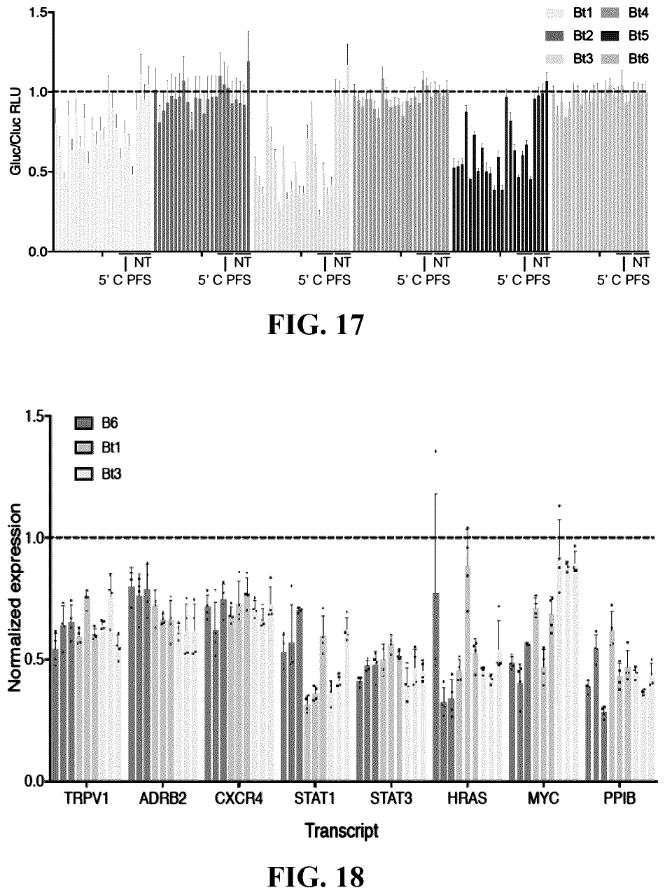

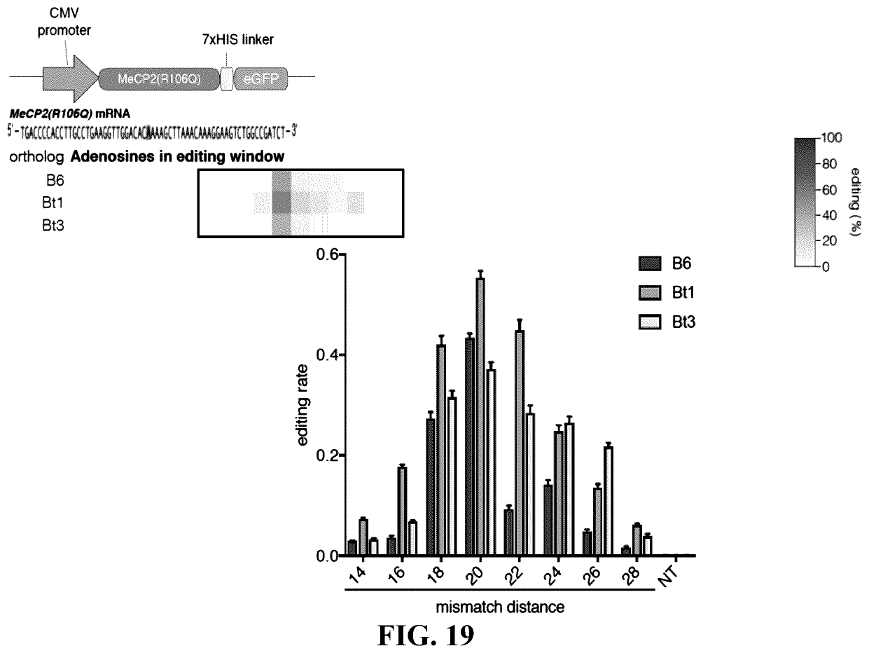

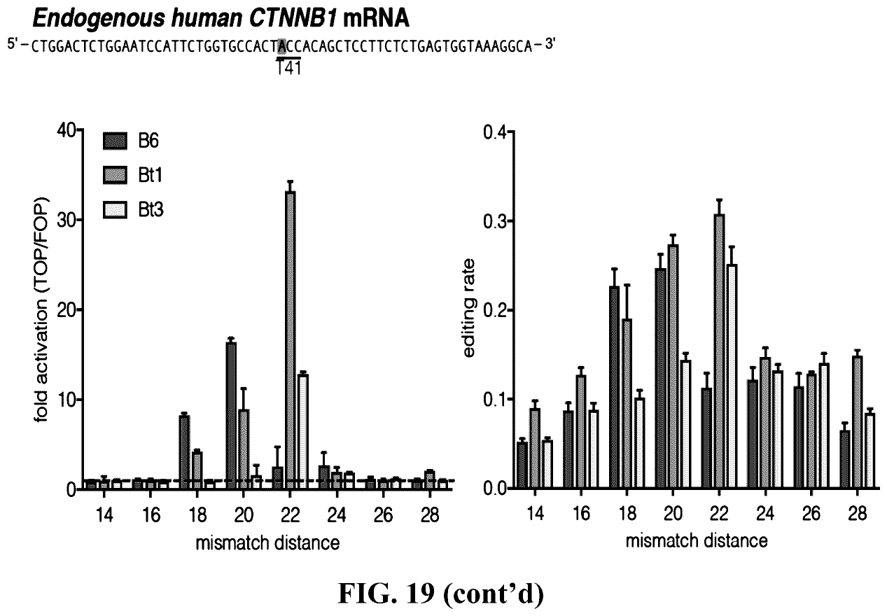

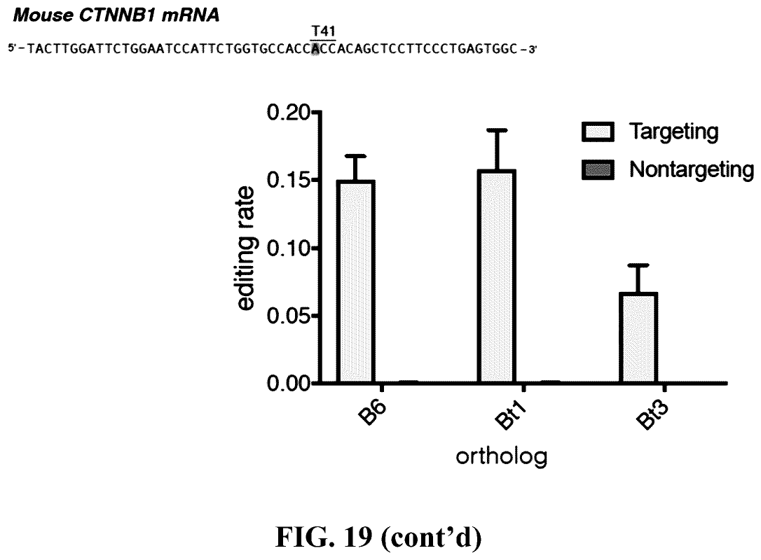

E. coli essential gene screens were performed to determine protospacer flanking sites (PFS) as shown in FIG. 10. E. coli essential gene PFS screens showed depletion by Cas13b-t1, Cas13b-t3, and Cas13-bt5 (FIG. 14). The tested active orthologs had 5′ D PFS preference (FIG. 15). Cas13b-t5 robustly depleted sequences containing PFS in E coli (FIG. 16). Active orthologs from PFS screens also mediated gene knockdown in mammalian cells (FIG. 17). The Cas13b-ts' mediated knockdown of endogenous transcripts in mammalian cells (FIG. 18). Cas13b-t1 and Cas13-t3 were found to mediate A-to-I RNA editing in human cells (FIG. 19).

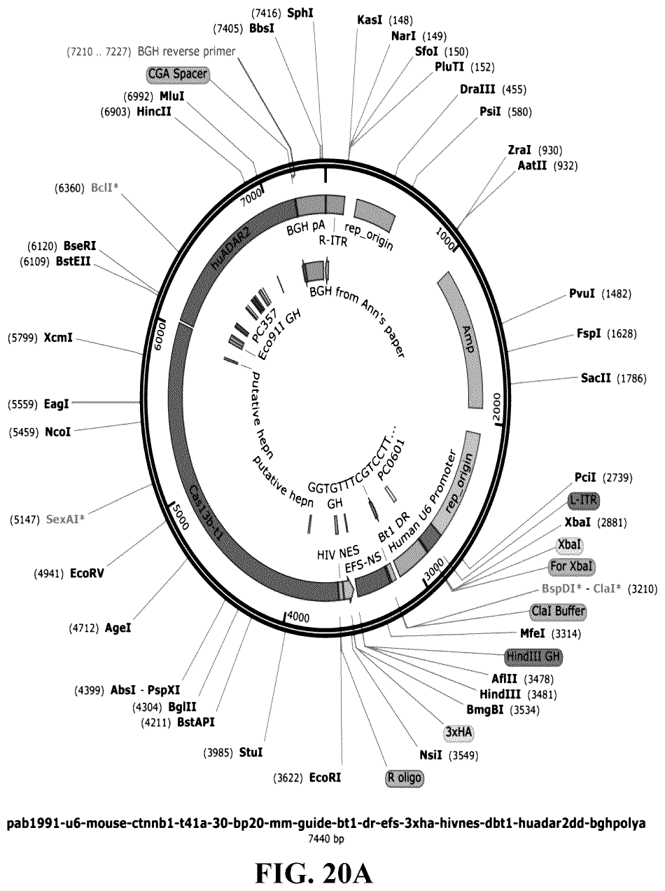

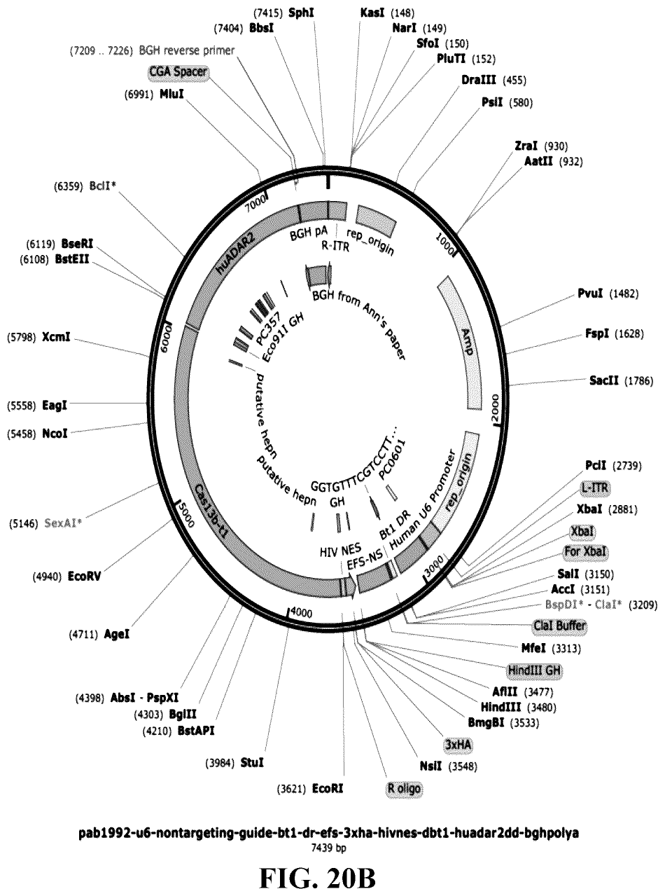

Injected C57BL/6 male mice are injected with single vector AAV containing Cas13bt1-huADAR2dd(E488Q) fusion driven by EFS promoter and guide RNA targeting the mouse beta catenin gene or nontargeting guide RNA. Maps of the vector expressing targeting guide RNA is shown in FIG. 20A and the vector expressing the non-target guide RNA is shown in FIG. 20B. Sequences of the vectors are shown in the Table 12 below.

TABLE 12

| | |
|---|---|
| pab1991-u6- | ggccgcaggaacccctagtgatggagttggccactccctctctgcgcgctcgctcgctcactgaggccgggcgaccaa |
| mouse-ctnnb1- | aggtcgcccgacgcccgggctttgcccgggcggcctcagtgagcgagcgagcgcgcagctgcctgcaggggcgcctga |
| t41a-30-bp20- | tgcggtattttctccttacgcatctgtgcggtatttcacaccgcatacgtcaaagcaaccatagtacgcgccctgtag |
| mm-guide-bt1- | cggcgcattaagcgcggcgggtgtggtggttacgcgcagcgtgaccgctacacttgccagcgccctagcgcccgctcc |
| dr-efs-3xha- | tttcgctttcttcccttcctttctcgccacgttcgccggctttccccgtcaagctctaaatcgggggctccctttagg |
| hivnes-dbt1- | gttccgatttagtgctttacggcacctcgaccccaaaaaacttgatttgggtgatggttcacgtagtgggccatcgcc |
| huadar2dd- | ctgatagacggtttttcgccctttgacgttggagtccacgttctttaatagtggactcttgttccaaactggaacaac |
| bghpolya | actcaaccctatctcgggctattcttttgatttataagggattttgccgatttcggcctattggttaaaaaatgagct |
| SEQ ID NO: | gatttaacaaaaatttaacgcgaattttaacaaaatattaacgtttacaattttatggtgcactctcagtacaatctg |
| 5247 | ctctgatgccgcatagttaagccagccccgacacccgccaacacccgctgacgcgccctgacgggcttgtctgctccc |
| | ggcatccgcttacagacaagctgtgaccgtctccgggagctgcatgtgtcagaggttttcaccgtcatcaccgaaacg |
| | cgcgagacgaaagggcctcgtgatacgcctatttttataggttaatgtcatgataataatggtttcttagacgtcagg |
| | tggcacttttcggggaaatgtgcgcggaacccctatttgtttatttttctaaatacattcaaatatgtatccgctcat |
| | gagacaataaccctgataaatgcttcaataatattgaaaaaggaagagtatgagtattcaacatttccgtgtcgccct |
| | tattcccttttttgcggcattttgccttcctgtttttgctcacccagaaacgctggtgaaagtaaaagatgctgaaga |
| | tcagttgggtgcacgagtgggttacatcgaactggatctcaacagcggtaagatccttgagagttttcgccccgaaga |
| | acgttttccaatgatgagcacttttaaagttctgctatgtggcgcggtattatcccgtattgacgccgggcaagagca |
| | actcggtcgccgcatacactattctcagaatgacttggttgagtactcaccagtcacagaaaagcatcttacggatgg |
| | catgacagtaagagaattatgcagtgctgccataaccatgagtgataacactgcggccaacttacttctgacaacgat |
| | cggaggaccgaaggagctaaccgcttttttgcacaacatgggggatcatgtaactcgccttgatcgttgggaaccgga |
| | gctgaatgaagccataccaaacgacgagcgtgacaccacgatgcctgtagcaatggcaacaacgttgcgcaaactatt |
| | aactggcgaactacttactctagcttcccggcaacaattaatagactggatggaggcggataaagttgcaggaccact |
| | tctgcgctcggcccttccggctggctggtttattgctgataaatctggagccggtgagcgtggaagccgcggtatcat |
| | tgcagcactggggccagatggtaagccctcccgtatcgtagttatctacacgacggggagtcaggcaactatggatga |
| | acgaaatagacagatcgctgagataggtgcctcactgattaagcattggtaactgtcagaccaagtttactcatatat |
| | actttagattgatttaaaacttcatttttaatttaaaaggatctaggtgaagatcctttttgataatctcatgaccaa |
| | aatcccttaacgtgagttttcgttccactgagcgtcagaccccgtagaaaagatcaaaggatcttcttgagatccttt |
| | ttttctgcgcgtaatctgctgcttgcaaacaaaaaaaccaccgctaccagcggtggtttgtttgccggatcaagagct |
| | accaactctttttccgaaggtaactggcttcagcagagcgcagataccaaatactgtccttctagtgtagccgtagtt |
| | aggccaccacttcaagaactctgtagcaccgcctacatacctcgctctgctaatcctgttaccagtggctgctgccag |
| | tggcgataagtcgtgtcttaccgggttggactcaagacgatagttaccggataaggcgcagcggtcgggctgaacggg |
| | gggttcgtgcacacagcccagcttggagcgaacgacctacaccgaactgagatacctacagcgtgagctatgagaaag |
| | cgccacgcttcccgaagggagaaaggcggacaggtatccggtaagcggcagggtcggaacaggagagcgcacgaggga |
| | gcttccagggggaaacgcctggtatctttatagtcctgtcgggtttcgccacctctgacttgagcgtcgatttttgtg |
| | atgctcgtcaggggggcggagcctatggaaaaacgccagcaacgcggcctttttacggttcctggccttttgctggcc |
| | ttttgctcacatgtcctgcaggcagctgcgcgctcgctcgctcactgaggccgcccgggcgtcgggcgacctttggtc |
| | gcccggcctcagtgagcgagcgagcgcgcagagagggagtggccaactccatcactaggggttcctgcggcctctagA |
| | gagggcctatttcccatgattccttcatatttgcatatacgatacaaggctgttagagagataattggaattaatttg |
| | actgtaaacacaaagatattagtacaaaatacgtgacgtagaaagtaataatttcttgggtagtttgcagttttaaaa |
| | ttatgttttaaaatggactatcatatgcttaccgtaacttgaaagtatttcgatttcttggctttatatatcttgtgg |
| | aaaggacgaaacaccgAGCTGTGGcGGTGGCACCAGAATGGATTCCGCTGAAGAAGCCTCCGATTTGTGGGGTGATTA |
| | CAGCTTTTTTATCGATCTCGtaggtcttgaaaggagtgggaattggctccggtgcccgtcagtgggcagagcgcacat |
| | cgcccacagtccccgagaagttggggggaggggtcggcaattgatccggtgcctagagaaggtggcgcggggtaaact |
| | gggaaagtgatgtcgtgtactggctccgcctttttcccgagggtgggggagaaccgtatataagtgcagtagtcgccg |
| | tgaacgttctttttcgcaacgggtttgccgccagaacacaggaaacttaa |
| | gcttgccaccATGTACCCATACGATGTTCCAGATTACGCTTATCCCTACGACGTGCCTGATTATGCATACCCATAT |
| | GATGTCCCCGACTATGCCggatccCTTCAACTGCCTCCACTTGAAAGACTGACACTGgGATCCGAATTCGAGAA |
| | CATCAAGAAAACCAGCAACAAAGAGGTGTACAGCATCGAGCAGTACGAGGGCGAGAAGAAGTGGTGCTTC |
| | GCCATCGTGCTGAACAGAGCCCAGACCAACCTGGAAGAGAACCCCAAGCTGTTCGAGCAGACCCTGACCAG |
| | ATTTGAGAAGATCATGAAGCAGGACTGGTTCAACGAGGAAACGAAGAAGCTGATCTACGAGAAAGAGGAA |
| | GAAAACAAAGTCAAAGAGGAAATCCAGATCGCCGCCAGCGAGCGGCTGAAGAACCTGgcAAACTACTTCAG |
| | CgcCTACCTGCACGCCCCTGACTGCCTGATCTTCAACCGGAACGACACCATCCGGATCATCATGGAAAAGGC |
| | CTACGAGAAGTCCCGCTTCGAGGCCAAGAAGAAGCAGCAAGAGGACATCTCCATCGAGTTCCCCGAGCTGT |
| | TTGAGGAAGAGGACAAGATCACCTCTGCCGGCGTGGTGTTCTTCGTCAGCTTTTTTATCGAGCGGCGGTTCC |
| | TGAACCGGCTGATGGGCTATGTGCAGGGCTTCAGAAAGACAGAGGGCGAGTACAACATCACCAGACAGGTG |
| | TTCAGCAAGTACTGCCTGAAGGACAGCTACAGCGTGCAGGCCCAGGATCACGATGCCGTGATGTTCAGAGA |
| | CATCCTGGGCTACCTGAGCAGAGTGCCCACCGAGATCTACCAGCACATCAAGCTGACCCGGAAGCGGAGCC |
| | AGGATCAGCTGAGCGAGAGAAAGACCGACAAGTTCATCCTGTTCGCCCTGAAGTACCTCGAGGACTACGGA |
| | CTGAAGGACCTGGCCGATTACACCGCCTGCTTTGCCAGAAGCAAGATCAAGAGAGAGAACGAGGACACCAA |
| | AGAGACAGACGGCAACAAGCACAAGTTCCACCGCGAGAAGCCCGTGGTGGAAATCCACTTCGACAAAGAG |
| | AAGCAGGATCAGTTCTACATCAAGCGGAACAACGTCATCCTGAAGGCCCAGAAGAAAGGCGGCCAGAGCA |
| | ACGTGTTCCGGATGGGAGTGTACGAGCTGAAGTATCTGGTGCTGCTGAGCCTGCTGGGCAAAGCCGAAGAG |
| | GCCATCCAGAGAATCGACCGGTACATCAGCAGCCTGAAGAAACAGCTGCCCTACCTGGATAAGATCAGCAA |
| | CGAAGAGATCCAGAAGTCCATCAACTTTCTGCCCAGATTCGTGCGGAGCCGGCTGGGACTGCTGCAAGTGG |
| | ATGATGAGAAGAGACTGAAAACCCGGCTGGAATACGTGAAGGCCAAGTGGACCGATAAGAAAGAGGGCAG |
| | CAGAAAGCTGGAACTGCACCGGAAGGGCAGAGATATCCTGCGGTATATCAACGAGAGATGCGACAGACCC |
| | CTGAGCCGGAAAGAGTATAACAACATTCTGAAGTTCATCGTGAACAAGGACTTCGCCGGCTTCTACAACGA |
| | GCTGGAAGAACTGAAGCGGACCAGACGGCTGGACAAGAACATCATCCAGAAGCTGAGCGGCCACACCACA |
| | CTGAACGCTCTGCACGAGCGAGTGTGTGACCTGGTCCTGCAAGAGCTGGGCTCTCTGCAGTCCGAGAACCTG |
| | AAAGAGTACATCGGACTGATCCCCAAAGAAGAGAAAGAAGTCACCTTCCGCGAGAAAGTGGACAGAATCC |
| | TGGAACAGCCTGTGGTGTACAAGGGCTTCCTGAGATACGAGTTCTTCAAAGAAGATAAGAAATCCTTCGCC |
| | AGGCTGGTGGAAGAGGCTATCAAGACCAAGTGGAGCGACTTCGACATCCCTCTGGGCGAAGAGTACTACAA |
| | CATCCCTAGCCTGGACAGATTCGACCGGACCAACAAGAAGCTGTACGAGACACTGGCCATGGACCGGCTGT |
| | GTCTGATGATGGCCAGACAGTACTACCTGCGGCTGAACGAGAAGCTGGCCGAGAAGGCACAGCACATCTAC |
| | TGGAAAAAAGAGGACGGCCGGGAAGTCATCATCTTCAAGTTCCAGAATCCGAAAGAGCAGAAGAAGTCCTT |
| | CAGCATCAGGTTCAGCATCCTGGACTACACCAAGATGTACGTGATGGACGACCCCGAGTTCCTGAGCAGGC |
| | TGTGGGAGTACTTCATCCCTAAAGAGGCCAAAGAGATCGACTACCACAAGCACTACGCCAGAGCCTTCGAC |
| | AAGTACACCAACCTGCAGAAAGAAGGCATCGACGCCATCCTGAAACTGGAAGGCAGAATCATCGAGCGCC |
| | GGAAGATCAAGCCCGCCAAGAACTACATCGAGTTTCAAGAGATTATGAATCGGAGCGGCTACAACAACGAC |
| | CAGCAGGTTGCCCTGAAGAGAGTGgcGAACGCCCTGCTGgcCTACAACCTGAACTTCGAGAGAGAGCACCTG |
| | AAGCGGTTCTACGGCGTCGTGAAGAGAGAGGGCATCGAGAAAAAATGGTCCCTGATCGTGGGAGGGTCAG |
| | GCGGTAGTcagctgcatttaccgcaggttttagctgacgctgtctcacgcctggtcctgggtaagtttggtg |
| | acctgaccgacaacttctcctcccctcacgctcgcagaaaagtgctggctggagtcgtcatgacaacaggca |
| | cagatgttaaagatgccaaggtgataagtgtttctacaggaacaaaatgtattaatggtgaatacatgagtg |
| | atcgtggccttgcattaaatgactgccatgcagaaataatatctcggagatccttgctcagatttctttata |
| | cacaacttgagctttacttaaataacaaagatgatcaaaaaagatccatctttcagaaatcagagcgagggg |
| | ggtttaggctgaaggagaatgtccagtttcatctgtacatcagcacctctccctgtggagatgccagaatct |
| | tctcaccacatgagccaatcctggaagaaccagcagatagacacccaaatcgtaaagcaagaggacagctac |
| | ggaccaaaatagagtctggtCaggggacgattccagtgcgctccaatgcgagcatccaaacgtgggacgggg |
| | tgctgcaaggggagcggctgctcaccatgtcctgcagtgacaagattgcacgctggaacgtggtgggcatcc |
| | agggatcActgctcagcattttcgtggagcccatttacttctcgagcatcatcctgggcagcctttaccacg |
| | gggaccacctttccagggccatgtaccagcggatctccaacatagaggacctgccacctctctacaccctca |
| | acaagcctttgctcagtggcatcagcaatgcagaagcacggcagccagggaaggcccccaacttcagtgtca |
| | actggacggtaggcgactccgctattgaggtcatcaacgccacgactgggaaggatgagctgggccgcgcgt |
| | cccgcctgtgtaagcacgcgttgtactgtcgctggatgcgtgtgcacggcaaggttccctcccacttactac |
| | gctccaagattaccaagcccaacgtgtaccatgagtccaagctggcggcaaaggagtaccaggccgccaagg |
| | cgcgtctgttcacagccttcatcaaggcggggctgggggcctgggtggagaagcccaccgagcaggaccagt |
| | tctcactcacgTAAcgacctcgactgtgccttctagttgccagccatctgttgtttgcccctcccccgtgcc |
| | ttccttgaccctggaaggtgccactcccactgtcctttcctaataaaatgaggaaattgcatcgcattgtct |
| | gagtaggtgtcattctattctggggggtggggtggggcaggacagcaagggggaggattgggaagacaatag |
| | caggcatgctggggatgcggtgggctctatgg |
| pab1992-u6- | ggccgcaggaacccctagtgatggagttggccactccctctctgcgcgctcgctcgctcactgaggccgggcgacca |
| nontargeting- | aaggtcgcccgacgcccgggctttgcccgggcggcctcagtgagcgagcgagcgcgcagctgcctgcaggggcgcct |
| guide-bt1-dr- | gatgcggtattttctccttacgcatctgtgcggtatttcacaccgcatacgtcaaagcaaccatagtacgcgccctg |
| efs-3xha- | tagcggcgcattaagcgcggcgggtgtggtggttacgcgcagcgtgaccgctacacttgccagcgccctagcgcccg |
| hivnes-dbtl- | ctcctttcgctttcttcccttcctttctcgccacgttcgccggctttccccgtcaagctctaaatcgggggctccct |
| huadar2dd- | ttagggttccgatttagtgctttacggcacctcgaccccaaaaaacttgatttgggtgatggttcacgtagtgggcc |
| bghpolya | atcgccctgatagacggtttttcgccctttgacgttggagtccacgttctttaatagtggactcttgttccaaactg |
| SEQ ID NO: | gaacaacactcaaccctatctcgggctattcttttgatttataagggattttgccgatttcggcctattggttaaaa |
| 5248 | aatgagctgatttaacaaaaatttaacgcgaattttaacaaaatattaacgtttacaattttatggtgcactctcag |
| | tacaatctgctctgatgccgcatagttaagccagccccgacacccgccaacacccgctgacgcgccctgacgggctt |
| | gtctgctcccggcatccgcttacagacaagctgtgaccgtctccgggagctgcatgtgtcagaggttttcaccgtca |
| | tcaccgaaacgcgcgagacgaaagggcctcgtgatacgcctatttttataggttaatgtcatgataataatggtttc |
| | ttagacgtcaggtggcacttttcggggaaatgtgcgcggaacccctatttgtttatttttctaaatacattcaaata |
| | tgtatccgctcatgagacaataaccctgataaatgcttcaataatattgaaaaaggaagagtatgagtattcaacat |
| | ttccgtgtcgcccttattcccttttttgcggcattttgccttcctgtttttgctcacccagaaacgctggtgaaagt |
| | aaaagatgctgaagatcagttgggtgcacgagtgggttacatcgaactggatctcaacagcggtaagatccttgaga |
| | gttttcgccccgaagaacgttttccaatgatgagcacttttaaagttctgctatgtggcgcggtattatcccgtatt |
| | gacgccgggcaagagcaactcggtcgccgcatacactattctcagaatgacttggttgagtactcaccagtcacaga |
| | aaagcatcttacggatggcatgacagtaagagaattatgcagtgctgccataaccatgagtgataacactgcggcca |
| | acttacttctgacaacgatcggaggaccgaaggagctaaccgcttttttgcacaacatgggggatcatgtaactcgc |
| | cttgatcgttgggaaccggagctgaatgaagccataccaaacgacgagcgtgacaccacgatgcctgtagcaatggc |
| | aacaacgttgcgcaaactattaactggcgaactacttactctagcttcccggcaacaattaatagactggatggagg |
| | cggataaagttgcaggaccacttctgcgctcggcccttccggctggctggtttattgctgataaatctggagccggt |
| | gagcgtggaagccgcggtatcattgcagcactggggccagatggtaagccctcccgtatcgtagttatctacacgac |
| | ggggagtcaggcaactatggatgaacgaaatagacagatcgctgagataggtgcctcactgattaagcattggtaac |
| | tgtcagaccaagtttactcatatatactttagattgatttaaaacttcatttttaatttaaaaggatctaggtgaag |
| | atcctttttgataatctcatgaccaaaatcccttaacgtgagttttcgttccactgagcgtcagaccccgtagaaaa |
| | gatcaaaggatcttcttgagatcctttttttctgcgcgtaatctgctgcttgcaaacaaaaaaaccaccgctaccag |
| | cggtggtttgtttgccggatcaagagctaccaactctttttccgaaggtaactggcttcagcagagcgcagatacca |
| | aatactgtccttctagtgtagccgtagttaggccaccacttcaagaactctgtagcaccgcctacatacctcgctct |
| | gctaatcctgttaccagtggctgctgccagtggcgataagtcgtgtcttaccgggttggactcaagacgatagttac |
| | cggataaggcgcagcggtcgggctgaacggggggttcgtgcacacagcccagcttggagcgaacgacctacaccgaa |
| | ctgagatacctacagcgtgagctatgagaaagcgccacgcttcccgaagggagaaaggcggacaggtatccggtaag |
| | cggcagggtcggaacaggagagcgcacgagggagcttccagggggaaacgcctggtatctttatagtcctgtcgggt |
| | ttcgccacctctgacttgagcgtcgatttttgtgatgctcgtcaggggggcggagcctatggaaaaacgccagcaac |
| | gcggcctttttacggttcctggccttttgctggccttttgctcacatgtcctgcaggcagctgcgcgctcgctcgct |
| | cactgaggccgcccgggcgtcgggcgacctttggtcgcccggcctcagtgagcgagcgagcgcgcagagagggagtg |
| | gccaactccatcactaggggttcctgcggcctctagAgagggcctatttcccatgattccttcatatttgcatatac |
| | gatacaaggctgttagagagataattggaattaatttgactgtaaacacaaagatattagtacaaaatacgtgacgt |
| | agaaagtaataatttcttgggtagtttgcagttttaaaattatgttttaaaatggactatcatatgcttaccgtaac |
| | ttgaaagtatttcgatttcttggctttatatatcttgtggaaaggacgaaacaccGTAATGCCTGGCTTGTCGACGC |
| | ATAGTCTGGCTGAAGAAGCCTCCGATTTGTGGGGTGATTACAGCTTTTTTATCGATCTCGtaggtcttgaaaggagt |
| | gggaattggctccggtgcccgtcagtgggcagagcgcacatcgcccacagtccccgagaagttggggggaggggtcg |
| | gcaattgatccggtgcctagagaaggtggcgcggggtaaactgggaaagtgatgtcgtgtactggctccgccttttt |
| | cccgagggtgggggagaaccgtatataagtgcagtagtcgccgtgaacgttctttttcgcaacgggtttgccgccag |
| | aacacaggaaacttaagc |
| | ttgccaccATGTACCCATACGATGTTCCAGATTACGCTTATCCCTACGACGTGCCTGATTATGCATACCCATATGA |
| | TGTCCCCGACTATGCCggatccCTTCAACTGCCTCCACTTGAAAGACTGACACTGgGATCCGAATTCGAGAACAT |
| | CAAGAAAACCAGCAACAAAGAGGTGTACAGCATCGAGCAGTACGAGGGCGAGAAGAAGTGGTGCTTCGCC |
| | ATCGTGCTGAACAGAGCCCAGACCAACCTGGAAGAGAACCCCAAGCTGTTCGAGCAGACCCTGACCAGATT |
| | TGAGAAGATCATGAAGCAGGACTGGTTCAACGAGGAAACGAAGAAGCTGATCTACGAGAAAGAGGAAGAA |
| | AACAAAGTCAAAGAGGAAATCCAGATCGCCGCCAGCGAGCGGCTGAAGAACCTGgcAAACTACTTCAGCgcC |
| | TACCTGCACGCCCCTGACTGCCTGATCTTCAACCGGAACGACACCATCCGGATCATCATGGAAAAGGCCTAC |
| | GAGAAGTCCCGCTTCGAGGCCAAGAAGAAGCAGCAAGAGGACATCTCCATCGAGTTCCCCGAGCTGTTTGA |
| | GGAAGAGGACAAGATCACCTCTGCCGGCGTGGTGTTCTTCGTCAGCTTTTTTATCGAGCGGCGGTTCCTGAA |
| | CCGGCTGATGGGCTATGTGCAGGGCTTCAGAAAGACAGAGGGCGAGTACAACATCACCAGACAGGTGTTCA |
| | GCAAGTACTGCCTGAAGGACAGCTACAGCGTGCAGGCCCAGGATCACGATGCCGTGATGTTCAGAGACATC |
| | CTGGGCTACCTGAGCAGAGTGCCCACCGAGATCTACCAGCACATCAAGCTGACCCGGAAGCGGAGCCAGGA |
| | TCAGCTGAGCGAGAGAAAGACCGACAAGTTCATCCTGTTCGCCCTGAAGTACCTCGAGGACTACGGACTGA |
| | AGGACCTGGCCGATTACACCGCCTGCTTTGCCAGAAGCAAGATCAAGAGAGAGAACGAGGACACCAAAGA |
| | GACAGACGGCAACAAGCACAAGTTCCACCGCGAGAAGCCCGTGGTGGAAATCCACTTCGACAAAGAGAAG |
| | CAGGATCAGTTCTACATCAAGCGGAACAACGTCATCCTGAAGGCCCAGAAGAAAGGCGGCCAGAGCAACGT |
| | GTTCCGGATGGGAGTGTACGAGCTGAAGTATCTGGTGCTGCTGAGCCTGCTGGGCAAAGCCGAAGAGGCCA |
| | TCCAGAGAATCGACCGGTACATCAGCAGCCTGAAGAAACAGCTGCCCTACCTGGATAAGATCAGCAACGAA |
| | GAGATCCAGAAGTCCATCAACTTTCTGCCCAGATTCGTGCGGAGCCGGCTGGGACTGCTGCAAGTGGATGAT |
| | GAGAAGAGACTGAAAACCCGGCTGGAATACGTGAAGGCCAAGTGGACCGATAAGAAAGAGGGCAGCAGAA |
| | AGCTGGAACTGCACCGGAAGGGCAGAGATATCCTGCGGTATATCAACGAGAGATGCGACAGACCCCTGAGC |
| | CGGAAAGAGTATAACAACATTCTGAAGTTCATCGTGAACAAGGACTTCGCCGGCTTCTACAACGAGCTGGA |
| | AGAACTGAAGCGGACCAGACGGCTGGACAAGAACATCATCCAGAAGCTGAGCGGCCACACCACACTGAAC |
| | GCTCTGCACGAGCGAGTGTGTGACCTGGTCCTGCAAGAGCTGGGCTCTCTGCAGTCCGAGAACCTGAAAGA |
| | GTACATCGGACTGATCCCCAAAGAAGAGAAAGAAGTCACCTTCCGCGAGAAAGTGGACAGAATCCTGGAAC |
| | AGCCTGTGGTGTACAAGGGCTTCCTGAGATACGAGTTCTTCAAAGAAGATAAGAAATCCTTCGCCAGGCTG |
| | GTGGAAGAGGCTATCAAGACCAAGTGGAGCGACTTCGACATCCCTCTGGGCGAAGAGTACTACAACATCCC |
| | TAGCCTGGACAGATTCGACCGGACCAACAAGAAGCTGTACGAGACACTGGCCATGGACCGGCTGTGTCTGA |
| | TGATGGCCAGACAGTACTACCTGCGGCTGAACGAGAAGCTGGCCGAGAAGGCACAGCACATCTACTGGAAA |
| | AAAGAGGACGGCCGGGAAGTCATCATCTTCAAGTTCCAGAATCCGAAAGAGCAGAAGAAGTCCTTCAGCAT |
| | CAGGTTCAGCATCCTGGACTACACCAAGATGTACGTGATGGACGACCCCGAGTTCCTGAGCAGGCTGTGGG |
| | AGTACTTCATCCCTAAAGAGGCCAAAGAGATCGACTACCACAAGCACTACGCCAGAGCCTTCGACAAGTAC |
| | ACCAACCTGCAGAAAGAAGGCATCGACGCCATCCTGAAACTGGAAGGCAGAATCATCGAGCGCCGGAAGA |
| | TCAAGCCCGCCAAGAACTACATCGAGTTTCAAGAGATTATGAATCGGAGCGGCTACAACAACGACCAGCAG |
| | GTTGCCCTGAAGAGAGTGgcGAACGCCCTGCTGgcCTACAACCTGAACTTCGAGAGAGAGCACCTGAAGCGG |
| | TTCTACGGCGTCGTGAAGAGAGAGGGCATCGAGAAAAAATGGTCCCTGATCGTGGGAGGGTCAGGCGGTAG |
| | Tcagctgcatttaccgcaggttttagctgacgctgtctcacgcctggtcctgggtaagtttggtgacctgac |
| | cgacaacttctcctcccctcacgctcgcagaaaagtgctggctggagtcgtcatgacaacaggcacagatgt |
| | taaagatgccaaggtgataagtgtttctacaggaacaaaatgtattaatggtgaatacatgagtgatcgtgg |
| | ccttgcattaaatgactgccatgcagaaataatatctcggagatccttgctcagatttctttatacacaact |
| | tgagctttacttaaataacaaagatgatcaaaaaagatccatctttcagaaatcagagcgaggggggtttag |
| | gctgaaggagaatgtccagtttcatctgtacatcagcacctctccctgtggagatgccagaatcttctcacc |
| | acatgagccaatcctggaagaaccagcagatagacacccaaatcgtaaagcaagaggacagctacggaccaa |
| | aatagagtctggtCaggggacgattccagtgcgctccaatgcgagcatccaaacgtgggacggggtgctgca |
| | aggggagcggctgctcaccatgtcctgcagtgacaagattgcacgctggaacgtggtgggcatccagggatc |
| | Actgctcagcattttcgtggagcccatttacttctcgagcatcatcctgggcagcctttaccacggggacca |
| | cctttccagggccatgtaccagcggatctccaacatagaggacctgccacctctctacaccctcaacaagcc |
| | tttgctcagtggcatcagcaatgcagaagcacggcagccagggaaggcccccaacttcagtgtcaactggac |
| | ggtaggcgactccgctattgaggtcatcaacgccacgactgggaaggatgagctgggccgcgcgtcccgcct |
| | gtgtaagcacgcgttgtactgtcgctggatgcgtgtgcacggcaaggttccctcccacttactacgctccaa |
| | gattaccaagcccaacgtgtaccatgagtccaagctggcggcaaaggagtaccaggccgccaaggcgcgtct |
| | gttcacagccttcatcaaggcggggctgggggcctgggtggagaagcccaccgagcaggaccagttctcact |
| | cacgTAAcgacctcgactgtgccttctagttgccagccatctgttgtttgcccctcccccgtgccttccttg |
| | accctggaaggtgccactcccactgtcctttcctaataaaatgaggaaattgcatcgcattgtctgagtagg |
| | tgtcattctattctggggggtggggtggggcaggacagcaagggggaggattgggaagacaatagcaggcat |
| | gctggggatgcggtgggctctatgg |

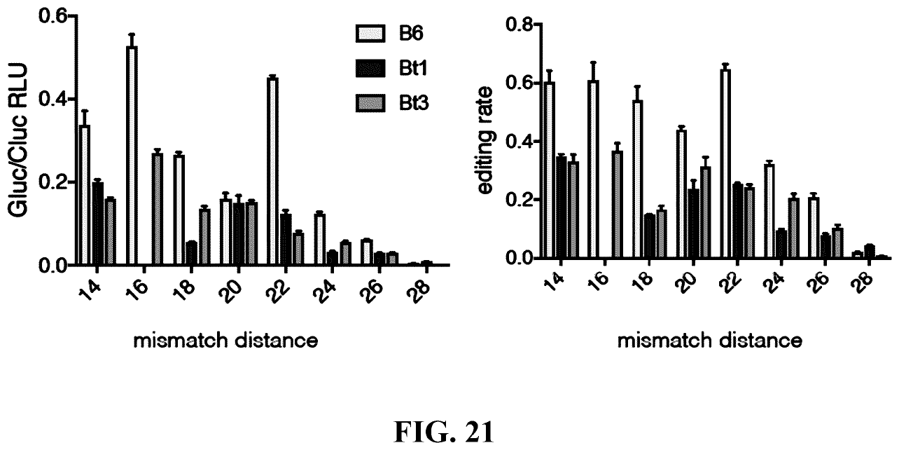

Cas13b-t1 and Cas13b-t3 mediated C-to-U editing of the reporter transcripts in mammalian cells when fused to evolved CDAR (FIG. 21).

Nuclei acid sequences of loci and related sequences of Cas13b-t1, Cas13b-t2,Cas13b-t3, Cas13b-t4, Cas13b-t5, and Cas13b-t6 are shown in Table 13 below.

TABLE 13

| Locus | | Sequences | |
|---|---|---|---|
| Cas13 | Locus | 1 | cgggcctggt ttttatttcg tcaatcgagc gactaagaaa tcttcaaagg cgcttaaatc |
| b-t1 | sequence | 61 | cttccatacc gtggcacagt taatggtttt ggctttgtta tctattacgg tgtatccata |
| | SEQ ID | 121 | gtcggtaacc cgaatgccga gtttttcggg ctcattttag acatttgcat ctatgccgcc |
| | NO: 5249 | 181 | ggcagcgctg aaggtttttt cggagctaat tgagtattca gcataaatgt tgaacggttt |
| | | 241 | tgccaatgcg ggtactatga tgttgatgct aacgttgata aatacaaatg tgatggtccc |
| | | 301 | tcccataggg cctgtcggcc tggactatat cgcaggagcc gtcagggcag ccgggaacca |
| | | 361 | ggcagacgta gttgatttat gtcttgctga tgacccgtca aagactctcc agggctattt |
| | | 421 | cgctacgcac agcccgcaat tggtgggggt ctcttttcgc aatgtggacg attctttctg |
| | | 481 | gccaagcgcc cggtggttcg tccccgacct ggctgacact atccgtacga tacgaagtat |
| | | 541 | gacggatgca ccaattgtag ttggcggcgt tggcttttcc attttttccg agcgaatcgt |
| | | 601 | cgaatatacc ggcgctgact ttgggattcg gggcgacgga gagcaggcaa tagtttcact |
| | | 661 | tcttaatcag ctgcagcggc cggaacggct tgaacgcata gatgggttag tccggcggcg |
| | | 721 | cgacggagtt attcacagca accgaccagc gtggcctgca ccgctttctt tgcgcaccga |
| | | 781 | acgtgatgcg attgataacc tcgcttactt caaaaaagga gggcagtgtg gtgtggagac |
| | | 841 | caaacggggc tgtaaccgcc gatgcctata ttgtgccgac ccgctggcta agggtgcggc |
| | | 901 | agtcaggccg agggccccgt cggaggtcgc cgatgaggtc cagtctctaa taggcaaggg |
| | | 961 | aatagaagta ttgcatttgt gcgactctga gttcaacatc tctcaaagcc acgcctatgc |
| | | 1021 | ggtctgcgaa gagttcagcc gtcgctcatt tgcgaaaaag gtgcgctggt acacatatat |
| | | 1081 | ggcggtggtg ccattcgatg ccgagcttgc cggggctatg agcagagcgg gctgtgtcgg |
| | | 1141 | tatcgacttt accggcgact ctgcgtgccc atcaattcta aagacctatc gccagcggca |
| | | 1201 | tcataaagaa gaccttgcct cggcggtgcg tttgtgccgt gctaacggca taacggttat |
| | | 1261 | gatagacctg ctgtttggcg gcccgggtga aacgccggaa acggtcgcag agacaataga |
| | | 1321 | tttcattaag caaattgacc cggattgcgc aggggctccg ctcggtataa gaatctaccc |
| | | 1381 | cggcaccgaa atggcccgaa tagtggcaaa cgaaggccca ccggaaacga acccgaacgt |
| | | 1441 | tcaccgaaag tacgaggggc ctgtggattt cttcaaacca acttactata tatctgaagc |
| | | 1501 | cctcggtgag cagccggccg ggcttatcaa ggatttgatt tcggcagatg aaagattctt |
| | | 1561 | tgagccgatg ccggaaatag ccccggaggc tctaaaaagt agccagtcca ccgaccacaa |
| | | 1621 | ttacaatgat aataccgaac ttgtagaagc aatcagcaaa ggtgcacgcg gggcatattg |
| | | 1681 | ggatatactg cgcaagcttc gctgcgacta agcagcttat ggtagtagat gattcccgcc |
| | | 1741 | tgcgggagat tggcccgaat cctgaggaat ttgttagaag cggatgcaat gttgattttt |
| | | 1801 | ggggtaaaaa cgggggcagg gggatttggt ccccggtttg aggattccga gaagcccacc |
| | | 1861 | cgtagggatc tccgctccct tagggataaa ttcgcttcga gtttgaaatt ggtccccggt |
| | | 1921 | ttgaggattc cgagaagctc acccgtagtg atctccgctt cgcttcggct ttgtttgggt |
| | | 1981 | ttgttttccc cgcgtctgcg aagtggttca ttttcataat cctttataac atataagttt |
| | | 2041 | acgttcattt tgggctttcg gcaaattggg tttgaattgg gtttgttttt ttggactgcg |
| | | 2101 | aaatcatctt tttttctgta aacctttgtt ataagagagt ttacattcat ttgggcattt |
| | | 2161 | agtaaattgg gtttgattgg ctttgaattg ggtttgtttt caccaagtgt ccaattggat |
| | | 2221 | ttattttcat aatcctttgt attatatgga tttacgttca tttgagcatc cagaaaattg |
| | | 2281 | gctttgtttt gcataaaaag ggctgatttg tagaggactc tttacagttg tagagggcaa |
| | | 2341 | gttagttaag agtgagctaa agtgcctaaa gtgaactaaa gttggattct cgattctcgt |
| | | 2401 | atagcgtata gcgtatttca cggttattca ccattcatta aggaataaat ttgattaggc |
| | | 2461 | ctgctggccc ctccggcgat tagtaaatgg ttctcggcgg caaaacaacg cgcctctata |
| | | 2521 | attgggcgaa catgcacgtt tgagtcgaaa attggtgctt tcttgacagg ataaacagga |
| | | 2581 | gtaactcgtt gtgagaaaag gagtaaaatt ttttttcaat tttccgattt taggttccaa |
| | | 2641 | ctacctgcac ttttgattga aaaatcacaa atgtcttgcc tattttaacg cagtttttcg |
| | | 2701 | tcgaaacgtc agcgaactag gaaaataggc gatttctggg ggaaaacaaa taaaaaatgc |
| | | 2761 | acaaaagtga caaaaaaacg gccaaaaaag tgcttttttt ggctgccttt accccgtgag |
| | | 2821 | atgatttacc aaaccttcct ctgctattcc tatgcaagtt tgctcagggc tggtgtgaat |
| | | 2881 | actataaaaa tttgtgctgt aatcactcca caaatcggag gcttcttcag cgtggaaatt |
| | | 2941 | ctggaggcca aaatgaaata cgctgtaatc accccacaaa tcggaggctt cttcagcttc |
| | | 3001 | actacctctc aaatcgccca actatacgct gtaatcaccc cacaaatcgg aggcttcttc |
| | | 3061 | agctcgcaag tcccgtccac gcacaaagtt tgagctgtaa tcaccccaca aatcggaggc |
| | | 3121 | ttcttcagca tgagcttttg gttgtgctgg atatgccagc tgtaatcacc ccacaaatcg |
| | | 3181 | gaggcttctt cagcacaaaa cggttcaaca aggtcgaaga actagctgta atcaccccac |
| | | 3241 | aaatcggagg cttcttcagc ttctgcggag tctttcgccg gtgttcaaat gctgtaatca |
| | | 3301 | ccccacaaat cggaggcttc ttcagcctat cctttataat acattttcct atatagattt |
| | | 3361 | acaatacaaa acccacgaca aaactgactt cttcttttga atcatgccgt attataacac |
| | | 3421 | ttttttacac tatcaaagac cacttttttt ctattccttc tcttttcacg accccataga |
| | | 3481 | atctcttcag atgttccctc tcaaaattga gattatagtg caaaagcgca tttcgcaccc |
| | | 3541 | gctttaaagc aacctgttga tcattattat aaccgcttct attcattatc tcctgaaatt |
| | | 3601 | ctatataatt ttttgctggt ttaatctttc ttcgttcgat aatccttcct tcaagcttta |
| | | 3661 | gtattgcatc gattccctct ttttgaaggt ttgtatattt gtcgaacgcc cttgcatagt |
| | | 3721 | gcttatggta gtctatttct tttgcttctt ttgggataaa atattcccaa agtctgctta |
| | | 3781 | aaaattcagg atcgtccatt acatacatct ttgtataatc caagatcgaa aagcgtatcg |
| | | 3841 | aaaaactctt cttttgctct tttggatttt ggaatttgaa aataatcact tctctgccat |
| | | 3901 | cttccttctt ccaatagata tgctgtgcct tttctgcaag tttttcgttc aatctgagat |
| | | 3961 | aatattgcct tgccatcata aggcaaagtc tgtccattgc cagtgtttca tatagcttct |
| | | 4021 | tgtttgttct gtcaaatcga tcaagagatg ggatgttata atactcttca ccaagaggaa |
| | | 4081 | tatcaaaatc cgaccacttt gtcttaattg cttcttcaac aagtctggca aaactctttt |
| | | 4141 | tgtcttcttt gaagaattcg tatctcaaaa atcccttata aacaaccggc tgttccaaaa |
| | | 4201 | tcctatctac cttttctcta aaagttacct ctttttcttc tttaggtatc agcccaatat |
| | | 4261 | attccttgag attctccgat tgcaaactgc ccagttcttg tagaaccaaa tcacataccc |
| | | 4321 | tttcatgaag tgcattgagc gttgtatgcc cggaaagctt ctggataata tttttgtcta |
| | | 4381 | atcgtctggt tcttttcagt tcttcaagtt cattataaaa tccggcgaag tctttgttca |
| | | 4441 | ctataaactt taaaatatta ttatattcct tcctgctaag tggcctatcg catcgctcgt |
| | | 4501 | tgatatatct gagtatatcc cttccttttc gatgtagttc aagcttcctc gatccctctt |
| | | 4561 | ttttatccgt ccacttggcc ttaacatatt ccaatcgagt ctttaacctt ttctcatcat |
| | | 4621 | caacctgtaa aagacctagt cttgaacgta cgaatcttgg aaggaagttt atagattttt |
| | | 4681 | gaatctcctc attacttatt ttatctaaat aaggcaactg cttctttaaa ctactaatat |
| | | 4741 | agcggtcaat tctttgaatt gcctcttcgg cttttcccaa tagactcaaa agaacaagat |
| | | 4801 | atttaagttc ataaactccc atcctgaata cgttggactg tccacctttt ttttgagcct |
| | | 4861 | tcagaataac attatttcgt ttaatataaa attggtcttg cttctctttg tcaaaatgaa |
| | | 4921 | tctcgactac cggcttttcc ctgtgaaatt tgtgtttgtt accatctgtc tctttcgtat |
| | | 4981 | cttcgttctc ccttttaatt ttacttcttg caaaacatgc tgtgtagtct gccaaatcct |
| | | 5041 | taagtccata atcctcaaga tatttcagtg caaataatat gaacttgtcc gtctttcttt |
| | | 5101 | cgctcaactg atcctggctt ctctttcgag ttagtttgat atgctgatat atctcagtgg |
| | | 5161 | gaactcggga caggtatccg agaatatccc tgaacataac tgcatcatgg tcctgcgcct |
| | | 5221 | gaaccgaata actatcctta agacaatatt tggaaaaaac ttgccgtgtt atattatatt |
| | | 5281 | caccctctgt ttttctaaac ccttggacat atcccattaa gcgatttaaa aatcttcttt |
| | | 5341 | caataaaaaa tgagacaaag aatactacac ctgctgatgt tatcttatct tcttcttcaa |
| | | 5401 | ataactctgg aaattcaatc gaaatatctt cttgttgttt tttcttcgct tcaaaacggc |
| | | 5461 | tcttttcgta tgctttttcc ataattatcc ttatggtgtc atttcgattg aatatcaggc |
| | | 5521 | agtcaggcgc gtgaagataa tgtgagaaat aattccttaa attctttagt ctttcactgg |
| | | 5581 | ccgctatttg aatttcttct tttactttgt tttcctcttc tttttcataa atcagttttt |
| | | 5641 | ttgtttcctc attaaaccaa tcctgtttca tgattttttc aaatcttgta agtgtttgct |
| | | 5701 | caaataactt tggattttcc tctaaatttg tttgtgctct attaagaact attgcaaaac |
| | | 5761 | accacttttt ttctccttca tattgctcga tagaatacac ttctttattg cttgtttttt |
| | | 5821 | ttatattttc aaactccata tttttaatcc ccgatcttct tactaatata aagtcaaagt |
| | | 5881 | ggtgcgaaat atcgcaccct accctctgcc tgcgcaggaa agaccaacgg acgattcgat |
| | | 5941 | gcagtttgag ctgccaggtg cttttatcat tcaaggggta aaatagcaga aaagccttaa |
| | | 6001 | tgtgtcaagg ggattttaga tttactattt ccaatttacg attttggatt gagattgctt |
| | | 6061 | cggcctaaaa gacaggcctc gcaatgaccc ccttagagtt gaaagcactc taaacaaggg |
| | | 6121 | ggcaggcggg gggaatatcg aatatcgaat ctgaatgtcc aatgtcgaag tgcaactgcg |
| | | 6181 | cgggaatgac aggtcggcagatttatttaattctgtggcc agatccctcc gcttcgtcca |
| | | 6241 | cctgcggtgg acttcggtcg ggatgacact gggggtgtgc cattgctccg ctcg |

| | Relevant | GACATAAAGACCCAATGGTGGCGTATGGGCTTGGCATACGTCTGATGTTTCATCAGAAAATCCTTTCTGG | |
|---|---|---|---|
| | sequence | AGGCTCTTGACTTATGGAATGAAATGCCGATAATGATATTAACCGGATTCCGTTCCGGCCTCTAAAACTT | |
| | SEQ ID | GCTTCCGTGAGTTTTAGAGGGTTCACAATACACGGGGCTGCTCTCTCGTAAAGGGCAGCCCTACTCTTTT | |
| | NO: 5250 | GGTTCATCCTGTTTGTGTGGGGTATAAAGCTTTAAAAATCCTTCAATAAAGGTTATATCTTCAGAGTTTA | |
| | | ACTTGCCATAGATTACCAAAGTAGCGTTTCCGCTGGGTAGGGGTATTGAAGTGGGTGTTGAACCAGTCG | |
| | | AATCGCCATTTTTAGAGCTATAAGAGGGGGTCGGTTGCATTGGCGGCGATCCCGCTTCATATTCTTCCAT | |
| | | TTCTTCTTTTACTCCATTAACTTCAGTCTTTCCTAATTGTTCTTTGATAAACATATTTGTGTCTCGAAAGCA | |
| | | TTCGATTGTCGTTTGCGCACCTTCATCTGTGAATCCACGTTGCTTGAGTGACCATATAAGGGCATCGTCC | |
| | | GAGGGTAAGTTTTCGGTATAACTTTGATATAACTCATCAAATATGGTAGGTGACTTAGCACATCTCTCTA | |
| | | ACGCTTCTTTTCTTTCTAATGAATTATGAGGAGCTTCTGTTATTATAAAAGCATCCTCCGAAATACCAAC | |
| | | CTTACCCTTTTGGTAACGAATAAGTCCATATTGAGCCATAGCAGCTAATATTGTAAGAGAACGACCGTGT | |
| | | AGCCTATTATAACCCCATACCTTAACTGCAATCTCTTTTGCCACAAATGCCTTTTTTTCCCTCTCATACAA | |
| | | TGCCCGTACATTTTTAACGCTTTCTCGCAAGCTGATACACGGATACCGTGGGCTACGTGGCCTATATTTC | |
| | | CTTCTTTTCTGTTGTTTTGTTTCAATTTTTTTGTCTTTTTGTCCTTTTTGTTCATTCATAATATTTACCCTTA | |
| | | AATTTTCTGGTCTTTTATATATCATCGACAACCACCGCGTGAAATACAAGAGAAATATTACTTTTTTCGT | |
| | | GAAATTTATGAAATTCTTTGTGATAAAGTGTTGCCGGAGAGTAATGTGCTGTTATAACAGATAAAAAGC | |
| | | CCCGCTGGGGCCTTGCAAACGACCCAACGGGGCAAAATGGCGGGTTAGCTGAGCTGCTGAAATAAACC | |
| | | TCGTGTTTCTTATTTCGAGTGCCCCTTCGGCTCTGCTCCCCCCTTCGCTATCCTCCTTCGCCAAGGCTACG | |
| | | AAAGAACCAGCTACGGAGGGCAGGCAGGGCTTCGGCTAATTCAGGACGGCATCCGTAAACTTTAAATTA | |
| | | TCAGATTTTCGTCTGCTTGTCATGAAGAAATAATGGTGGGGCAAGCCCACCCTACGATTTTGGTGTTTGT | |
| | | CGCGTATTATCCGGGAAATCCGGGCTAAGACAACGCCAATCAGGTTAATACACTGACGGCTTCGGCGAT | |
| | | TAGCTCGCGTATCGGCAGATGCTGTGGGCAGCGAGCTTCCGCAAGTCTGTAATCGGTGGTGAGCAGTTT | |
| | | GTTTCTAACCTTGCGGGGAATTTGAGCGAAAAGCCCTCTTGCCCTATCCTGGTCGCTGTAGCTGTTGTAG | |
| | | TACATCAAATACCGCATAATGTCACTGACATAAGGTGTATCGGGCAGGGCTGAATCGCAGATGTATGCA | |
| | | CAGCCGGCACAATAACCGCTGCAGGTCGCCTCCGCGTATTTCTTGAAAACGTCTAAATCCTGCTGAGTG | |
| | | AGTTTTGTTTTGTTCAGCACGGCATCGACGTTTGTAGTTAAAAGAGATATATTCTGCATCCCGACGCAGA | |
| | | CTGCGCTGAACCTTTTATCTTGCAGCACAACCTTTATCTTTGCCTGTTCTTCGGTGAAACCCCGCTTCTGG | |
| | | AAGTGACCGAGGAGTTTCTTATCCTGTTCGGTTTCAACCTCCTGAAGACGTCTGACCCCGCGGGAGAAA | |
| | | GCCTTCATAGCAGTAAGACCTATGCCCGCTTTGTGGCAGGCTTCAATAGCAGCCTGCATTGGAGCATCCT | |
| | | GCATGACTCGGAAGTTGTAAAGTGTCGTTATTGCATCAATCCAGTCGAGCTTGGCTGCAGCGGCGAGGC | |
| | | ATCTGGCCATATTCGAGTGGGTGCTGAACCCGAAAAACCGTATCAGCCCGCGCTTCTTTGCATCTTTGGC | |
| | | CCACTGTTTTAATTCATCCGTCAACTGTGCAGGGTCGGATAAGCCGTGAAAAGCATAGTACAAATCGAC | |
| | | GTATTTGGTGTTCATTCTTTTCAGCGAGGCCTGAAGACGCTCTTCCACCTTCTCGGCGGTTATTGCCCCCC | |
| | | AAAAAATCGCCGCCTTGCTTACAATGAATAGTTTCTTTCTGGCCTTCGGGTTCTTCGAGAGGAATTTACC | |
| | | TATCCCGAGCTCTGCGTTTCCACGCCCATAGTTGTAAGCTGTATCCCAATAGGTAACCCCCCATTCGAGG | |
| | | GCTTTTCTTAAAACGATTTGCTTATCAATAAAACTAAAGTCTCCGCCCAGAGACAGGCAGGAGACCTCG | |
| | | ACGCCTATTTTGCCTAACTTTCGTTTCGGCATCTGTGGGATTTTGGTTTTTTTTGGTTTTGGTGGAGCATT | |
| | | AGGGTCGCTGGTCTTCGGTTCGCCTGGGCCGGCTGTTGCTTCAGCCGAACCCCGTATTAAATACGGGGCC | |
| | | AATCCAGTCGCTCCCATCGTTTTCAAAAAATTCCTTCTGTTGATTTTGTTGTGTTTTTCCTTCATCATATTA | |
| | | ACCTCCGAAAGCGATTAATGTTTAAGTAATAACTTTCTATCTGCACTTCTTGTTTCATTTTCCGCAGAGAC | |
| | | CCTTATAGCTTTTTTGCCGCTTACGGGGCATTCGTGCTCACAAACTCCGCAGCCGATACATTTTTCAATAT | |
| | | CAACGAGAGGTCTTTGCAGGCGAACCTGAAGCTCAATTTTGCTGCCTGCAGCGGGGATTTTTTCGAATTG | |
| | | CTCATCAGGTGAAATTACGATGGTGTTTTCGGTGTTTGCTGCGATTTTTCTGCGTTCATCACCTTCGACGG | |
| | | CACAATAATAATCGCCGGTGGCAAATTTGGCAGGCACAATGTTCTCTTCAACTTCCACAGTATTATCCGT | |
| | | GGCTTTCTTTACAGTCAAAATGCCATCTCTTATTGTATTGAAACATTCCTCCGTATAAATTGCCTTTGGGC | |
| | | TAAGCGGGCAGTTTTCTTCGCAGACTATGCAGGGCTTATCCATCGCCCAGGGAAGGCACCTGTTTTGGTC | |
| | | AACAAAAGCGGTGCCCAGCTTGATTGGCCCGACGTCGGCAAACTCGTCGGTTCCGAGTTTTTCCGCAAG | |
| | | TGTAATCGGACGAATGGCCGAGGTCGGGCAAACCTGCCCGCAGGCCACGCAGTTTAGTTGACAGCCGCT | |
| | | TGAGCCGATTCGATTGTTCAGCACGGGGGTCCAGAGATTTTCCAGTCCTCCCTGTATGCCGCCTGGCTGG | |
| | | ATAACATTTGTGGGGCACACGCGCATACATTGGCCGCACTTGATACATCTCTTTAAGAATTCCTCTTCCG | |
| | | ACAATGCGCCTGGCGGGCGAATGACCTTATGGTACCAGTTGCTGCCGAGTTTGTTGCTCAGCCTGACCGC | |
| | | CGGTACTGCAATGATGCCGCCGGTGAGTGAAAGCACAAAGCCCCTGCGAGAGATGTCCGGATTTGTGAT | |
| | | TTCGCCTGCCAGTGACGGTTTTGTCTGGTAACTGATTAGTTCGTCCTTGCAGTCCCTGCGGCAATTAAAA | |
| | | CAAAGTACGCATTCACTTATTCTTATATTTCCGCTGGGCTGGCAGGCGCCTTCGCAGGCCCGCTCGCACA | |
| | | ATTTGCAATTGGTGCATTCGCTCCGGTTCTGGCTGATTCGCCATATAGCGAATCTGCCGAGGATGCCGAA | |
| | | TAATGCGCCGAGCGGACAAATGAATCTGCAGTAGAATCGGGGGATAGTCAGGTTAAGCAAGACCGCCG | |
| | | TCAGGAATATCGTTAGTATCAGCCATGCGCCCTCGTAAAAGCGTGCCGTTACTGAGGCGAGGTTGGCGG | |
| | | GTCTATCAGCAAGGGGCAATAATACGAGGTTAAAAGAGCGGGTTATCAACGGTATAGGGTCGAGCAGG | |
| | | CCCGTTTGCAAGGTAACCCCTATCGATGGAAACGCAGCCATAAAAAGAAAGAATATGAGAATGAGGTA | |
| | | TTTTATGCACTGGGCTTTTCGGTATTTGTTGAGCTGGATTTTTTGTGGTGCGGTTTTTTTTCGATTGCCTAA | |
| | | AAAGCCGACAAAGTGATGCAATGAGCCGAAGGGACAGACCCAACTGCAGAAGAACCGCCCGAATATTA | |
| | | TTGTCAATATAATCGTAGCCAGTGCCCATAAAAGAGGCCAGTAGAGGGTGTGTGTAGTTAATATTGTCC | |
| | | CGATTGCCACCAGCGGGTCGAGCTGTAAAAACCAGTTAACGGGCCAGCCCCGCAACTGCCACAATTTCT | |
| | | CGCCTACGGTGGCGACTATGCAGAACCAGGCGAACATCGAGAAAAAGAAGGCCTGGCTTATTTGTCTAA | |
| | | CTGTTACAATCTTCATACTTACACTTTTAGCTTACCGTGGCGGTTATTGGTTTGAGTGATTCATAATCAGT | |
| | | GGTTCCGGCATCGGCGATTTGGGCTTTGGCAAGGTAAGGCAGGTCCGAGGCCTTCAAATTTAGAAGACT | |
| | | GCAGCCATAGGCATCGGCCGCCACCATATCACAACTTGCAATCAGCGTATTTGCGCGTCTCAAATCTTCG | |
| | | ACAGAGCCTCCCGTTGGGCCATTGGTCATCATAACTTCAGTGCCGTCCAGTATAACCAACGTCGGCTTTA | |
| | | CCATCATAGCCAGCTCGGCAATGATTGTGTTAATATCCTGGTGAAAGATATTGCGGCGTCCGCCCAAAA | |
| | | GGCCGTACCAGTTTTTCATCGACATAGAAGCGCCGGCCCTGGCGTGGTGTTTGACCGGGGTGATACCAA | |
| | | TCAGTTTGTTGACCTTCTCAAACGGCCCGAAGAAGATAGGCCAATTTTTAATGAGTTTGCCACGTTCGAG | |
| | | CGTGGTGTGCTTGAAGTGATGGTCTTTTGGCAATATGACTTTTGCGCCTGCCCGGCTTGCCGCTTTACTG | |
| | | ATGCCGCTTAATGTAAAACAGCTTGCGGGGTCGTTAATCGGATTGTCGGTAACGTACACCCGCTTTGCCC | |
| | | CGGCTTTGTAACACAGCCGGACTACCTCAGCCACCAGTTCAGGGTGGGCCGTGGCTCCAAGCATTGGCG | |
| | | GCGAGGCGAATGCAACATTTGGTTTTATTGCAACCGTTTCGCCGGGCTTGACAAATCTTTTTATGCCGCC | |
| | | GAGCAATTCTATCGCCTTGTTGACAGTCACTCTCCTATCGGCGCCTTTGACAATGCTCATTGTCTTGCCCT | |
| | | CTTCAGACGGCACCGAAAAAGGCGGTAATGTTACGAGCGATTCGACATCAACGCCGGGTTTTGGGCCCT | |
| | | GGCTATCATAAAGCCGATAAGAAATTATACCGGCGGCGGCGATCGAGATTCCTGCTTTGCCTGCCCGCG | |
| | | CCAGAAATTTCCTTCGCCCGAGTTTTTTATCATTTTGCTCGGTCATTGTTAGCTGTCCCGCTGAGCTTATT | |
| | | AACAAGCATTACCGCTAAATTTTCCGCGGACTGTTGGTTTTCAGCTTCGTGAATGCCAACAACAAAAGG | |
| | | CCCTGTCGAAAGCACAATTTCGGTGGTGTCATAGAAATCCAGGACTTTGCCTTCGAGGGTTTTATTGGTT | |
| | | GCCTTCTTTGCTGTGGCGCCATTTTCAATCAGAAAGCTGCGATAGCTTTCTGCGACTGCCTCGGCATCTTT | |
| | | GGGACCGGAGCGTTTGCTCAGAAATGCCGTGATGGTTTCACCGTTAAGCTGGTATCCGGCAGCGAAGAT | |
| | | GTCAGTCAATCCTTCAAAGCCAAATGCACTTGCCAGATAAAGCTTAATGCTTCCTGGGACCAAATTATCC | |
| | | TTTGGCAGGTGCTCGATTTCAGGTATAGCGGTATCATCGTGAACGGCCAGGTTCGTGGGAATTTTCCTTG | |
| | | CGACTTCCGCCATTGCCGCAAACAGCTCATCCGATTCGGCGAAGCCGACCAGCTCGATATAATATTGGC | |
| | | CGTGCGCAAGATAAAACGCATTACTGGTTTTGTATGCAAATTGCATATCCGGCAGGTTCTCAACTTCGGG | |
| | | CCTTTTTTGCACGCTGTAAACCGAGAATGCGTTTCTGGTTCTGGCCATATCAAAGATATAGAGCTCCATC | |
| | | ACCAGGTTTTCATCCGCCTGGCTTACAAATCTCTGGGTGGACAATTTTATAAAACCAGCGTCGATATAAA | |
| | | GGGGGGCCTTGCCGTTAATCTTTTCGTAAAGATTTTCGGTGGTGTAGACTTCAATTTCTGAAAGCGTTTT | |
| | | GAATCCGTAAGGCAGAAGAAAAGTCAGGTCTTTCTTTTGTTTTGGCATCTGCTTTATGAATACCCCAACG | |
| | | GCGATAAGTAAGAGAATCGCTAATAAGCAGATGCCTATAACAGATTCGAGACGTTTTGCCCGGCTTGGT | |
| | | ACCGAACCCATAACCAACTCCAGTAATGACAAATTACTTGACTTTATAACCGGGCTGGATTATAATTTTT | |
| | | GCCGGTGTTGCTGTCAACCCCAAATGCTACAGGTGAAAAAGGCGAAGATAGATTTCTAACGAGGTTGAC | |
| | | AAAGCAGGTCAGGGCGTGTTATAATAGGTTGCTAAAGTAAAAAGGAGACTGAAATGATTGAATATGCA | |
| | | CAATATTTGGGGTTTTGGACGCCGGGCCCCCTTGAAATTGCTGTTATTGCGATTGTCGCTCTTCTGATATT | |
| | | CGGCAGACGGCTGCCTGAAATCGCCCGCAACGTAGGCAAGAGCCTGACTGAATTCAAGAAGGGGCTTC | |
| | | ACGAGGCCAAGGAGACCAAGGACGAATTGGTGGATGATGTCCGGGAAGTCAAGGATGATGTGGTAAGA | |
| | | GAGGCGAAGGATGCCGCCGGGCTGAATGAAGAGGATACAATGGGCTCTGATTGATTATTGATAAAGGG | |
| | | GAACTAATCACTGAGAACAATTGTCAATCATTAATCAACAATCAATATTGAAGATCCGCCTGTGGCGGA | |
| | | ATCAATTTTTAAGATGGGCGATACAAAGAAGAAAGAGGACCTCCTTGATTCCACTATGAGTCTGGGCGA | |
| | | CCACCTTGAGGAATTGCGGATGCGGCTGATTCGCGCGCTGGTGGGCCTGGCGTTAGCTCTTATTATCTGT | |
| | | CTGATCTTCGGCAAGCTGCTGATATCATTTATTCAAAAACCTTACGTTGCTGTGATGGGTGAAGAGGCTA | |
| | | CTCTGAAGACGCTTGCCCCGGCCCAAGGGATTAACAGCTACGTAAAAATAGCCTTGGTCTCAGGCTTGA | |
| | | TATTCTCATCGCCCTGGGTCTTCTACCAGTTATGGATGTTCGTGGCTGCAGGACTCTATCCTAATGAAAA | |
| | | AAGATATGTGTATGTAGCAGTACCTTTTTCGGTGGTATTATTTGTTGCCGGAGCTTTGTTTTTCATCTTTG | |
| | | TAGTGGCAGAAGTGTCTCTTGCTTTCTTAATAAAGGTCGACAGGTGGCTCGGACTGGAACCCGACTGGA | |
| | | CTTTCCCGAAGTATGTGACCTTTGTAACCACCCTGATGCTGGTATTTGGTGTTGCGTTTCAGACCCCGAT | |
| | | AGCTATTTTCTTTTTGAACAAGACAGGTCTGGTTTCAGTCCAGGCGTTACGGCGGTCAAGAAAATATGTA | |
| | | CTGCTACTTATCGTTGTAGTAGCAGCTATGGCGACTCCGCCTGATGTGGTTTCTCAAGTAACACTGGCGA | |
| | | TACCGTTGTATGTGCTGTTTGAATTAGGCATACTGCTGAGTTACTTTGCAGAACTAAAAAAGAGAAAGTC | |
| | | GAAAAACAACCAGTGATAAGCCGACAATCCCCAGCTTTCCCAGTACCGACTACTTGTTTCTTT | |
| Cas13 | Locus | 1 | agccgagtcg atggtagcta aggtgaacga caagcgtggt tatacggaga taagttgatg |
| bt-2 | sequence | 61 | cgactggtta tccatgagga cgaagtagat tcgatggatt ggttatatgg aattagatcg |
| | SEQ ID | 121 | aaacgtatac ctatgaagct cacagtcaaa taccaatcgg gatagaaatg cggcgcgcgc |
| | NO: 5251 | 181 | aaccttaggc aaaggcttgg ctgtttcagc gtttccgctt tacgtgcccg tttagccttc |
| | | 241 | accatagtcc acctttccgc aagcctccct gcgatcccgg acagtcggat ttcccaaatc |
| | | 301 | cggttctggt ctcggcccta tttgtcattt tctggataaa ggccttcctg tacaatttga |
| | | 361 | gacttaagtg ctagctcact tgcaccccat aattgtacag tttaccagta tcctcgttcc |
| | | 421 | gagagtccat ggcattcgtt ccagttcggt gcctggatgc acatgcctta ctcagaacca |
| | | 481 | ccgagtaccc agagcccctt tgtcaggcgt gggcgctacc cactacctgg atgactttga |
| | | 541 | aagtcacctc agaagacatt actcttcctt catagctcat acggactcat gcgccagacc |
| | | 601 | aaatccctcc caacgtcttg gttttccctt gtacgttagg tctttgcagg ttgtcgccag |
| | | 661 | tccctgctgg gaaacggccc ttcccgacat tatctctgca atccttgtat aggtgcaagg |
| | | 721 | acccttaccc cgcagcgtcc cttcggtgcc cttgcccgtt tcttcccgaa ggactgcggt |
| | | 781 | ctcacctcag gatttaaagg ttcgacacgc caattatccg tcgcaatgca acttcaacaa |
| | | 841 | cggggcaaat ttcggggctg cagtcattcc ataacgttca agctcctata cctgctatgc |
| | | 901 | cctgcggttg cacccaccac tgagcatata tgagctcagg gcagccgggc cgtttacacc |
| | | 961 | acgcatcgcc cggatggtta cccattccga gatgtggcat cgctacgtgc ctgaatcggg |
| | | 1021 | caactggcac gacgggactt tcacccgctg gattgcagcc ttgtcggctg ctccaaatcc |
| | | 1081 | ctgttgccac aaaaattttc tttgaggcat ccacgttacg acgtgtcggc cacgcttcgt |
| | | 1141 | agcttatctg aacagccttt cgacttcacg atgcattata atggacatcg tgaatctagc |
| | | 1201 | tatgtcatgg tcaatcgtag tgtggccagg ggccggccaa tcgagtatac ttgataaagt |
| | | 1261 | gttcatgaag ctgtattctt taatctccca agagtatgct tcgaacgttt aagaaatgac |
| | | 1321 | agatagtggt gaagtggtct gaaaacgggc ccgggaggcg aagacgtgag tacaggtaca |
| | | 1381 | gtgaaatggt ttaatgcaag aaggggatac ggttttattg tccccgatga tggcggagat |
| | | 1441 | gatttatttg ttcaccgttc ggacattaac acagaggact atgcatcgcg agattattaa |
| | | 1501 | ggtcggcaac gacatggttg ccgaccatat caatcataag ggcttggata atcgcaaggc |
| | | 1561 | caatttgcga gcggcgacga ttgcgcagaa tgcgtggaac cgccagcgca aaagaagcgg |
| | | 1621 | atttatgggc gtagtgtgga ataagcagat gaggaaatgg cgtgttaata tcagtcacga |
| | | 1681 | gggcacgtgc aggcatatcg gctacttcga tgatgaggtt gaagcggcga aggcgcacga |
| | | 1741 | ccgggcagcg aaaaaatatc acggagagtt cgcgagtttg aatttcacgc gttaaagcca |
| | | 1801 | catcacagcg agtccgacta tggcggacgc agcaatctta agcatatttg gctgcgcaat |
| | | 1861 | gtgttgcgcg ggttcctgct tggggcgagc tatcgaggtg taatcacccc acaaatcggg |
| | | 1921 | ggcttctcca gcgccgtaaa agttgataag aattttagat gcgccgtaat cacccctcaa |
| | | 1981 | atcgggggct tctccagcgc tgaccgaatt gataaaacca agagagcgct gtaatcaccc |
| | | 2041 | cacaaatcgg gggcttctcc agctgtacga caaatcataa cagaatattt gaagctgcaa |
| | | 2101 | tcaccccaca aatcgggggc ttctccagca aaatgagaca ccacgcttga cgtcactgtg |
| | | 2161 | ctgtaatcac cccacaaatc gggggcttct ccagcttcga gctatatctg gctcggtctg |
| | | 2221 | atttggctgt aatcacccca caaatcgggg gcttctccag cactggctta gcaagttcct |
| | | 2281 | ttgggcgttt cgctgtaatc accccacaaa tcgggggctt ctccagctaa tcgaagatga |
| | | 2341 | gaccgaagac tatcactgct gtaatcaccc cacaaatcgg gggcttctcc agctgattgg |
| | | 2401 | gaaagcactc cttacgcacg agagctgtaa tcaccccaca aatcgggggc ttctccagca |
| | | 2461 | catcctcgat aatacgttat ctcgattgga tttacaacag aaaaatcact gaaaatacca |
| | | 2521 | gggtttttgg tgcaatgcgc acacattaga acctgttttc atactgctaa agaccagcgt |
| | | 2581 | ttttcaatcc cttccctttt cataattcca cagaacctct taaaatctgc cctggcaaaa |
| | | 2641 | ttaaggttat gatgcaaaag cgcgtttcgc acacgtcgga gagctttctg gtcatcttca |
| | | 2701 | ttgtaggcgc ttttatccat tatctcgcta aatgggatgt agttctttgg aggtcttggc |
| | | 2761 | tgctctggac cgatagtctt tttctcaagc tcgagtatgg cttcaattcc ttccctttgc |
| | | 2821 | aggtttgtgt atctgttcat cccttgcgta taaagcctat ggtagtcgat ttgttcttcg |
| | | 2881 | tcttttggca aaaaataatc gcaaagtcgg gccaaaaact ccgcgtcgtc catcacatag |
| | | 2941 | agtttcgtat aatccctcag cgagaaccgt accgaacaac tctgctccgg ctgtgccgga |
| | | 3001 | ttcttcaagg tgaaaataat tacttcttcg ccatcctctt tcttccactc gatttgctgt |
| | | 3061 | gccctcttgg caagctcttt gttaagacta agatgatatt gccttgcgat catcagcaaa |
| | | 3121 | agcctgtcca ttgccagtgt ttcatacagt ctcttattgt ctttatcaaa cgtatcaagt |
| | | 3181 | gacacgattt cgtaatactc cccccccaga ggaacatcat aaacctctcc tttttccctc |
| | | 3241 | accgcttctt caacaagcct cgcaaaactc tttctgcttt ctctgaagaa ttcattcctc |
| | | 3301 | aaaaatcctt tataaataac cggctgtttc acaaccctgt ccgtcttttc ttcaaatgac |
| | | 3361 | acctcttttt cttctttggg tatcagtcca acatattcct tcagttcttc tctgcctagg |
| | | 3421 | ctttcaagtt cttgcacgac caaatcgcac accttttcgt gcagcgcatt gacgctttta |
| | | 3481 | tgcctggaaa gattgcacac gatgttctta tctatccgtc tggtcttctt caattcttca |
| | | 3541 | agctctcggt aaaacccgtc gaggtgctta gtgaccaaaa gctccaaaat acggttatat |
| | | 3601 | tcatcgatgt tcagcggcct ctcacaccgc tcattgatat acctcagaat atcccgtcct |
| | | 3661 | tttcgatgga gctgaagctc cctcgacttt tcttttttct ccaaccactt ggccttaaca |
| | | 3721 | taatcaactc gcgccttgat ctttttttca tcatcaaccc ctaagagacc cagatgggaa |
| | | 3781 | cgcacaaacc tcggaagaaa tctcacgtat ccttcaatct cttccgtgct tttcttctct |
| | | 3841 | atgtgaggca actggttgcg caagctatga acatacctgt cgattctttt gactgcctct |
| | | 3901 | gctccttttc ctaataagct cagtagaaca agatatttaa gctcgtagac gcccatcctg |
| | | 3961 | aatatatagg tttgggcgcc tttcttctga gttcgcagaa tgacgttatt gtgtttgata |
| | | 4021 | taaaatgggt ctccttcggc tctctcaaaa tgaatctcga ctctcggctt cttcctgtga |
| | | 4081 | ggtttctgct ccttgccatc tgtattttcg tcctgctccc gcttaatcct cgttctggca |
| | | 4141 | aaacatgctg tgtagtctgc caaatcctcc agcccgtaat cctcaagata gttcagcgca |
| | | 4201 | aacaatatga acttgtccgt ctttctttcg cttaactggg tttcgcttcg catttgcgat |
| | | 4261 | tctttgatac gctgatacga ctcactggga actcgtgaca aataccccag aatatcccgg |
| | | 4321 | aacatgaccg catcatgatc cggcgtctta accgaataac tgtccctaag acaatatgtc |
| | | 4381 | gaaaaaacgt cccgcgttat tttatattcc ccctctgtac gcgtaaaccc ctgaacatat |
| | | 4441 | cccattaacc gatttaggaa ccttctctca gcaaaaaatg acgcgaaaaa taccacacct |
| | | 4501 | gctgatgtta tcctgccgtt ctcttcgaac aactccccaa attcaatcga aatatcttcc |
| | | 4561 | tgttcctttt ttgcctgttc aaaacgcgcc ttttcgtacg ctttttccat aattatcctg |
| | | 4621 | accgggtcat tttgggtgaa tatcaggcag tcaggcgtat gaaaatagtg cgagaaataa |
| | | 4681 | ttcctcaaat ctctaagctt ttcatcagcc gctttttgaa tttcctcctc tacttcgttt |
| | | 4741 | tgttcttgtt ttgcatagat cagttttttc gtttcctggt caaaccaatc ttcctttctg |
| | | 4801 | atcctttcga atcgtgtcag cgtttcctcg aacaacttcg gattcccctg caaatttgtt |
| | | 4861 | tgcgccctat taagcactat cgcaaaacac cacttcttgg ccccctcata ttgctcgata |
| | | 4921 | gaatacattc cttggctgct tcctttcttg atattttcaa cctgcatatc tcagactctc |
| | | 4981 | ccaattgttg tttttcgcca tttttgttga agtccccgaa tgtcagtcta ttgggccagc |
| | | 5041 | tgagtcaacc cacaaggcac aatgtacata cagtctcgag tcatttcgag aagactttcc |
| | | 5101 | gctcgcccga taagataagc tttgagtatc tcacggggtg gacccgagca gataattcca |
| | | 5161 | catctcgtat ccggtgaagc tatccggcat aaattcgtgc ttagtgaatc gtgtttcgtg |
| | | 5221 | ttgatacggc tcccggctgc attcactttt cacggcagag aatatcgcaa aataaggcaa |
| | | 5281 | cagtcaaagg aaaaagggta aaaatggtga aatagatgag cgagcagtga attgttgtgg |
| | | 5341 | caagcaagcc gcaaatgaat ccttcggcca cgctc |

| | Relevant | AGGACAGGTTTGGGGGCAAATATTAGAAGGCGGTTATGCAGTTTTGTGGTCGCGGAGGCTATGGGGGAG | |
|---|---|---|---|
| | sequence | ATTGCTGGTTTCTGAGGCAAATCGGGGCGGTTATGCGGGTAAGATATTAAGGAAGGCCGGTCTTTGGTC | |
| | SEQ ID | ATATCAGTTACAAGGGGCTTGAAAGATTGGGCGAGGTTCGTTAGATTAGTGAACGTGATAGGCTGCCGA | |
| | NO: 5252 | ATATGATAAGGATTCTTGTAGGATGAAAGGACGCGAAGAAGATGAACGGGACGAGACAGACAATTAAG | |
| | | CTGTGGACGTGGGTATTGGTGGGGCTGGGCTGTCTGGTTGGCGGACGGCTGAACGCCGAGCCGTGGGTG | |
| | | GTTTTTGAGGGTGGCGAGGGGCCGGGCAAGGGAAGGCATATCGTTCTTGTCACCGGCGATGAGGAGTAC | |
| | | CGATCGGAAGACTCAATGCCACAACTGGCCAAGATCTTATCAGTGCAGCACGGCTTTAAGTGCACGGTG | |
| | | CTGTTTGCCATCAATAAGGAAACGGGCGAGATCGATCCCGTGACGGTGGACAACATTCCGGGTCTTGAA | |
| | | GCTCTGGGGCAGGCCGACCTGATGGTGCTTTTCACGCGTTTCCGCGAGCTTCCCGACGAGCAGATGAAA | |
| | | TACATTATCGATTACACCAATTCGGGCAGGCCGATCCTCGGGCTGAGAACGGCGACGCACGCATTCTTC | |
| | | TACAGCAAGCACAAGGATAGTCCGTACGCCAAATACAGCTTCCGCGACAGGCAGTTCGAGGGTGGGTA | |
| | | CGGACGTCAGGTTTTGGGTGAGACCTGGATTAACCATTACGGACATCACCAAAAAGAGAGTACGCGCGG | |
| | | ACTTATTGCGAAAGGCATGGAAAAGCATCCGATTGTGAAAGGCATTAAGGATGTCTGGGGCCCATCTGA | |
| | | TGTTTACGGCATTACGACGCTGAGCGGCGACAGCAAGCCACTTATCATGGGACATGTGCTAAAGGGTAT | |
| | | GGAGCCGGATGATGAGCCAAACCCGGAGAAGGAGCCGGTGCCGGTGGCCTGGACAAAATCTTACACAG | |
| | | GGGAAAAGGGCAAAACGGCCCGCGTTTTCGCTACGACCATGGGACACGGTGGCGACCTTAAGAACGAA | |
| | | GGTTTTCGCCGTCTTCTCGTAAATGCATGTTACTGGTGCATGGGAATGGAGGATAAGATTCCGGCCAAA | |
| | | AGCAAGGTTGATATTGTCGGCAAATACGAGCCCAATCCGATCGGGTTCGGCGGGCACAAAAAGGGCCT | |
| | | AAAGCCCTCGGATCATAAACTCTGATGAGCGGGCACAGCTCGGCATACTACGCAGGACAACAGCAAAC | |
| | | ATAGCAGGCGGGACAGAATAATCCTGGCCGCGTTTGCATCATACGGTGAAGGGTTCCCGCTATTCGCGC | |
| | | TGAACGCTACATCAGCCGGCGGCAACATCGAGAGCGATTTGAATTTCTCTCTGAAGATTTGCACATTTTG | |
| | | TGGATGTGGGGATTATTGTGTAGAATATCGATGATGGTGATGATAGCGGTGATGGTTAGCAGCGGTGCA | |
| | | CGAATTCGCAGGCTTCCTGCTACGGGCCGATAAGAAATCCGGCTGCTCCATCGAGCACCGCCGCGAACG | |
| | | TTATAAACGCTGCCTGGGGTCATCAGATTGGATTGAGGAAACAGCTCTTCCGATATAATCGTATGGTGCT | |
| | | TTTCGTAATTGTTTAGTCGTTTGAGAGGCCTTAGATGGGTGAAGAATCACGCAAGGTGGCGCTGCCCGG | |
| | | CAGGATTTACCAGAAGAAGAAGCGCTGGTGGTGGAACGTGCGGCTGCCCGGCGAGCAGAAGGCAAAAG | |
| | | CTCGCGCACTGAAGCCTGCTGGGTCGCGTTTGGCTACGACGGAGCGTGAGGTAGCTGAAGAGATTGCGC | |
| | | TGGAGATGTGGCAGCTCGCCATAAGGGGCGAAATTGAGGCAAAGGTCAAGGCGGAAGCTGAGCAAAAG | |
| | | ATTAGATCCTGCACGGAGGAGATAGAAAAAGTCAAAACCGAAGCGGCTGAAACGATAGCCAGGCTAAA | |
| | | GGCGGAATTCGAAGAAAAGGTAAGGACTTACTCCGAGGCGGTAGCCAGAGCCGAGGAAAAAGCGAAG | |
| | | GCAGAGGCCGAAGCAAGAGCACAAGTGGAAGCAAAGTTGAACGAGGTGCTTGCTGAGCCAACGGCAAC | |
| | | TGGTGCCTGCGAATGTTGTGGCAGAGAAGACGTTCCAGAGAATGACCTGGCGAGAATTGACTCGGGACA | |
| | | AATGCTTTGTCCGGACTGCATTAAGCAACTAAGAGGCTGATTCTCCTTTTATCGGCAGCTACACTTCGAG | |
| | | GCTCGACTCACTCAGCGATACAGCTAACACCGGCAGAAAGACTTTCTCACGATGAGCCGGAAGAGATCC | |
| | | CACCGGAGACAACAAACGCCATTGCTTGTGAACTTTCAACATCCAACGGCTTAACGGACTCTTTCGGCA | |
| | | AGAGCAAAACATTGCCGGCGAAGTTGTAAGATTGAGGCAGGTAGACTGCAACATGGTCTGAAAGTCCA | |
| | | AGGTTTTCAAGACTCTCTCTGGTAACAAAACCGACCACCTTTGCATCGGCTGCAGGCACAATGGTGGCG | |
| | | AGTACGGGTTTGTCAAAACTTTTCTTCTCACCAGCGAAGGCCTCAATCACGTCCTTAAGGGCTGAGAAA | |
| | | AGCAGCTTGACCAAAGGGAGCTTTTTGAAGAGCTTTTCTATGAGCTCAAAGAACTTCCTGCCTAAAAAG | |
| | | TTTGATGTGAGAAAGCCAATAACAATTATCAATACGACCGTCACCAGAAGTCCCAGCCCCGGGATTTCT | |
| | | ATTCGAAAGAGAGCCCGAAGGAAGGCATCTAAATTTGTGAATGCCCACACAAGCAGATAGACGGTGAG | |
| | | GGCTATCGGTACAAAGACCAGAAGTCCCTGGACGAAATAATGAATTAGTTTTTTCATTTTCTTGCTCCAA | |
| | | AAACGGGCCACTCAACCGATAAGGACGAAGAGTAAAGGCCAAAAGTGTTCTTTTCGCAGTTTCCTCAAG | |
| | | GAAGACGCATAACACCACTACCCATTTGACTCTGGCTGGGCACAGCACCATGTTCTCATTCAGCGAAAT | |
| | | ACCAAATTTGCGTAACCGGTGCAATGGCTTGGCCGGTAAATTGCCACGAAATGCTTTGATCCTTGAGTGT | |
| | | CCGGCGGTACCTCTCCGCCCGGCTTAGGTTGGGTGGGCTGTGGACAAGAAGAAAAGAAAAAAGCCCCT | |
| | | GTGCGGGCAGCAGCGAAGACATCCCCGTATTTCCATTTCTGTAATGTTTGAGATACTCGCCTTCTGCTTG | |
| | | ACAGGGGCAACATCAAGTCAATCTTATCTTCTTGCTTCTTAGCTCCCTTCTTACTACGCCCACATATAACC | |
| | | GCGGCTTGTTCTCGATGCCGGAAAGTAGTCAAGCAAGTCAAAATCGCTATGGCCGGAGATGTCACCTTT | |
| | | TAGAGTCACTTACTACTATATCCAGCCTAACATTTTGAATCGGCAGATTTGGTACTTGAGCTTGAGCTTT | |
| | | TCTGGATGTGGCCAATTTCGGCAATATGGCGGTGTTTTAACACAGGCAAAAAGGCCAAAAGTCAAAGAG | |
| | | CTTCCTATTTCTTTTCCGCTCCCCCAGACATCCAACTTGACGACATGGAGCCTTTCTCCCAAGCTCATCGA | |
| | | CTTCTCTGACTTAAACCCGATAACGAGAATTATTATTGTTCGGAATTTTTTCAGAAGACTTTAAGAATAC | |
| | | GCAAAAATTTTCTTCTTTTTGACCGAGAAGTCACTGATGAGCCAACTCGTCAGAGGCCGTGAATTGGAA | |
| | | AGACTTTTACCTTAATCGTTTCTATTGTGGCTTCTATTGCGGGCATCACCGTATCCCGCTGCTGGTTGTTT | |
| | | ACGGCAAGCGCTTTGTCCCGCAACGGGCCGTGCCATTGAGAAGTGCCGAGCATGACGCCGTCGATAATA | |
| | | GGATTCTTGCTCCGGTCACTATCGGCGCCGCATAGTATGGTCCAGGCAAGGGCCCAGGCCCTTACGGTG | |
| | | TTGTCGATGTCATATCCGCCGCCCCCAGTTGCAAGGATGGGTCTATTAAAACTCAGCACGGATTCGATAA | |
| | | CGCCGACATAAGCATTATTAGTTAAGCACAGGTGTGCAAGCGGGTCACCGGCAAGGGCATCAGCGCCC | |
| | | AACTGAAGAACGATTGCATCGGGATTGTAAGCGGCGATCAGTGGCGGTGCTATCGCCTTGAATGCCTTG | |
| | | ATGTATGCTTCGTCATAAGTACCCACCGGCAGAGGGACGTTTACACAGTAGCCCCTGCCCTGGCCGGTT | |
| | | CCGATCTCGTCGGCGAATCCGGTTCCGGGAAACAGTGCTCTCGGGTCTTCGTGAAATGATATAGTCATA | |
| | | ACATCGGAGCGGTCGTAAAACGCGTACCCAACGCCGTCTCCGTGATGAACGTCCACATCGAGGTATAAG | |
| | | ACTCGCTTTCCAGCCTCGGCGAGTATGATACAGGCAAGAGCGACATCATTGATGTAGCAAAAGCCGGAC | |
| | | GCGCGTTCGGGGCCTGCGTGATGAAATCCGCCCGATGGATTGAAGGCGACGTCAGCAGAGCCTGCGAG | |
| | | GATGAGCTTTGCACCGGTGAGTGTGGCACCAGAGGCCAGGACTGCATAGTCATATAGTCCCTTAAAGAC | |
| | | GGGACAATCGCTCGTCCCTAACCCCATATCGAGTGCCTCGGCATCCCAGCGGCCTTTTGAAGCGGTCTGC | |
| | | AAGGCGTGCAGGTATCGGGCTGAGTGGAATTTCTTCAGGACTATCCTATCAGCGGGCGCCGGCGGCACT | |
| | | TCGGTTCGGCCGCCGCCCGAAAGCAGGCCCATGGAATTCACAATCTTGCGAACTCTTTTAGCTCGGATC | |
| | | GTGTTAAAAGGATGGTCCGGGGGGTAAGGATGCTTTTCTAGCTCCCGCGAATAAACAAAGACAGCTTTT | |
| | | CTCATTGCCGGCATCGATCCACTGACAGACAGTTCTTCTTAAAGCGTCGACCACTCCAAATGACAGAAT | |
| | | ATAGTACTCTATGTGCCGCAAAACCGCAAAGCCTTTTGTTGTTCTTCAGCCTGTACAAACACCATTACTA | |
| | | TTCTTAAAATGCTCTCTTGAATGGCAAGAGAGTCAAAGGCGTATATCAACAGACAGCTCAAAGCAGGAA | |
| | | CGACTTGAGAGGCAAGCTTATGAAGCAGGCAGGGACCAAAGAGTTGGAGCAAATTCCAGGCGTAGGCG | |
| | | AAAGAATTGCTCAGGATATGCGAAATATTGGTATTCACTCTGTCAGCCAGCTCAAGGGCCGGGACCCTG | |
| | | AGAAGCTTTATCAAAGACTCTGCGATTTTAAGGCAAGCCCCGTTGACAGATGTATGCTCTACGTGTTGCG | |
| | | TTGTGCGGTATACTACGCGTCAAACACCGAGCATGAGCCAAATTTGCTGAAATGGTGGAATTGGAAGGA | |
| | | TAAAGACTGACTTCTTCTCGTGCTCTGACGCCGGCAGCAGTTCGAATCAGTTTTTATCGAGCGGGTCATA | |
| | | CCAGCGCTTAAGCACGCCTTCCCCCGTGGTTTCTTTCATATCACTTACATGGGTGTCCAGCCAAAGAATA | |
| | | TTCAAGGTTCCACCAGTTCTGAACTCGTCACCCGACTTTATGTTGTGCCGAAAGATCCCGCGGTACCAAT | |
| | | TCCTACGACTACCAGTTTCTCGATAGTGAGTCAGGTTTATGCCAGTTGGACTGGGGAAGAGCATGTCGG | |
| | | AGTTCCCGGCGTTCGCTCCATTTTCGATTCTTGGCTCCATGTGGTCATGGGCAACAATAACTTCAGTGTG | |
| | | TTTTGGAATAGCGTTTGTGTTCTCTTTGGTCAGCCAGCCATTGACAGCAAATGCCGAAGTGTAGATGAGT | |
| | | TTATAATCACCGCCATAGAGTAAATCCTTTGCTATCGTCTCGCAAAAGTTGCGGAAGGCTGCGCAGCCA | |
| | | AAGAGCTTTCTTTCTTTGACATAGTCTATATAGGCAATGCCCCAATAAGCGCGATCATCCGTGGCCCTGA | |
| | | GCGGATTGCCGGAGGAATTCCACCAAAGGTGGCCGTTACAGTTGTTGGTGTGCGTGCACATATCAGGCA | |
| | | TCGTGTATTCGTTGTCGTCAAGGTACATTCTAATACCCAGGCCTATACCTTTGAGGTGTCCCTTGCAGGC | |
| | | CTGGTCTCGTGCCTGATCCTTTGCTGCCTGCAGTGCAGGCATCAGAATTGCCAATAACAGAGCTATGATG | |
| | | GAGATAACCACCAACAGCTCTATTAGTGTAAACCCTCTTGCCATGTACTTAGCGAGCCTTGCGTTGTAAT | |
| | | TCATCATTGCGTTACCTCTTAGAGTTGTATCCATGTGTTAGCCCAAAGAATCATTCTTCGCAGGATACTC | |
| | | ACATCTCGGCATGAAGGCGGCGCCGCCATGCTGATATGCAGCTAACAGCCTACGTTTGGTAATTTGTTTC | |
| | | ACAACCTACCATTAAGCGTCAATATCGCTATGTTTGAGCGGTCCTTACCGCTTTAGAATACCAAATCTAC | |
| | | GTGAAAAAAGCAAGCCATTGTGGATTTCTTCAATAAATTTCCTCGCGGAAGGACCAAGTTTGCTGAATT | |
| | | CGCACCGGCTAACGTGCTTTCGGCGCGATTTTCGTGCGGTGGTCTAAGCCCGAGCCCTCCAGGTCAATGC | |
| | | TCACTGACGGGCCTATCTTCGCAAGGCCGGTATATCCGGCTCGATGTGAACGGTGATATTGACCGGGCG | |
| | | GGTCAACTGCTCGTCGAGGGCAGTCTCCAGACTCTCGGATATTTCATGGGCGGCGGCTATGTCTAAATTG | |
| | | GGGTCAACAAGTATATGCAGATCGAGAAAAACCTCCCTGCCAACCGTACGCGTCCTGAGCCTGTGCCAC | |
| | | TGGCGGATTTGCTTATTTGCATTGATTACACGCTCTATGTGCTCAACTGTTCCCTGGTCGACGGCACCCTC | |
| | | TGTCAGCTCGCGGAGGGAATCGCCGATTATCCTAAAGCCCACCCAGATAATCATGAGGCCAACCGCAAC | |
| | | GGCGGCTACCTGGTCGCCGTGCTCGAAGCCGAGCCTTAGCGATATGAAACCTATCACAACCGCCACGGA | |
| | | GCTCAAAGCATCACTGCGATGATGCCAGGCATTGGCATAAAGTGCGGCGCTGTGGGATTGGATGGCAGC | |
| | | TTTTTGCGTGACCCTGTAGAGCCACTCTTTAGCAGCGATCGAGACTACCGCGACGACCAGAATGGCGAT | |
| | | GCGAGGGATAGTAACTCGATTGGCGGCTATGGCAGCGGTGGCATAGTAGACCATTGCTCCACCGACAAG | |
| | | AATCAAGATGATAGCAATCGAGCCTGCCGAGAAGGTTTCAAGCCGGCCGTGGCCATAGGGATGTTCCCG | |
| | | GTCAGGCTCTTTGGAGCCGAGTCGTAAGCCTATGAGCACAACGACGTCCGTGGCCATGTCTGAAAGTGA | |
| | | GTGGAAGCCATCAGCTATCAGTGCTAAGGAAGCTGTGAAAAAGCCAATGACAATCTTGAGTGCTGAAA | |
| | | GCGCAATATTTATAGCGATGCCAAGATATGTGATCGACCTTATCTGCTTGCCGGCAATTTTTTGTTTGTC | |
| | | GTTTTGCATCAATCTGTTAGCCCAACACCTGGTTGCGTATTTTCAAAACGTCGTTGATATATCGCCTAAC | |
| | | GGTGGATTGGTCCTTTGCCTCCGCTATGACCCTTACTATAGGTTCGGTATTGCTGGCCCTGAGATGTAGC | |
| | | CAACCATCATCAAAATCGAATCGGCAGCCGTCGGTAGTGTCAAGCTTTGCATCGGTGAATGTCTCTTTT | |
| Cas13 | Locus | 1 | tatccaaaat gtggtttgaa ttcaagaatc aacgctttat tccttaaaaa ggggcggtgc |
| b-t3 | sequence | 61 | gatggaaaaa gaaccagaaa catccgtgca atcggcgtcg ggacacaata tggatatccc |
| | SEQ ID | 121 | gattgactgg tcggtaacct cacgctattt cgaagatgaa gatacgctga tgcaggtggt |
| | NO: 5253 | 181 | ggggatattt gctgaagact ctccgcagac cgtccgggac cttgccaagg ctatacagac |
| | | 241 | gcaaatatcc caggatgttc aattgcacgc tcacagcctg aagggagcct cggctcttat |
| | | 301 | cggggccgaa catctgcggc aaagagcctg gcggcttgaa tacgccgccc aggagaaaaa |
| | | 361 | cacggcggcg tttgaggcgc tgtttgacga gacaaaggcc gagttcgaca agctgatgtc |
| | | 421 | gttcctttac cgcgccgatt ggattgaagc agcaaaagaa cgccactgca acaggcaaca |
| | | 481 | ggccgagcag gtatgaaaca tcttttggaa aagaaggcga tggaatgagt ggatggttct |
| | | 541 | ccattttgat cattgatgat gacaggatgg ttacagacaa gttggagaag atcagcggcg |
| | | 601 | ccaaggctgc aaagaaaagg ttcagcctgg caggcgtttt ctcaaagggc gcctgaagcc |
| | | 661 | ctttatttgc aggcgtgcta ccgcttgtca acgggcaggg gacagaaccg caatcaggat |
| | | 721 | taccatcagt ttcttcattc cattaacctc gctttttcct ctcgttcttt ttcttcttcc |
| | | 781 | tggttttcgc agcgttgggc tgtctttttg ccggttttgt atagttgtcg ccgtaaatgt |
| | | 841 | caatgagtgc ggcttttagt ttttcgggcc agttgcggtt ttcaaaagcg cacacgagcg |
| | | 901 | gatcgccgct ttgtttcatc cagttatgaa gccggccctg catcttcttg atttcgctcc |
| | | 961 | tttgctctgc ggaatcgata aggttgttga ggcagtcggg gtcatttctg agatcataga |
| | | 1021 | actcctcggc cgcgcgatat ctgaacatct tgacgcgctg tgcggcgaac ttattcgttg |
| | | 1081 | gagcggcctc gaccatcgcc ttcatggtaa ggccctcgtt gttgtttcgg taccagaatc |
| | | 1141 | tgccgtcggc ccacggattg aagatatagc cgaagcgctt atcctggacg caccgcatcg |
| | | 1201 | ggacagcgtc tccgccggct ttcatgtcta tctgcgtaaa gaccacgtcg cgtccggatt |
| | | 1261 | gcttttcgcc tttcaacagc cccaggaaag aggaaccgtc aagccccctg ggtatgccca |
| | | 1321 | gaccgaccgc ttcgagcacc gtcgggaaga agtcgatccc tgagataaag tgcgccttat |
| | | 1381 | cgacggcgcc tgcttttacc atttgcggcc aacgaacgat ccacggcgtc cgcgtgctgg |
| | | 1441 | caagataggc gttgcatttt gcaaacggta tggcgatgcc gttgtcggag aggaacatca |
| | | 1501 | caagcgtatt ctcctcgaag cccgactcct tcagggcctg caacgtcttg ccgaaggtat |
| | | 1561 | cgtcgagtcg gcggacggag ttgagatagc agctcagttc ctgccgaacg cccggcaggt |
| | | 1621 | cgcagacaaa accgggaacc gcaacctcat cgggcttata cgtctttgaa ggttcctttg |
| | | 1681 | cccccttgat tggcttgccg ccgatatgat acgggcgatg cggatcgtgc gagttgacca |
| | | 1741 | taaagtagaa gggcttattc tcgcggcgac acctcgccag aaactccttg cagtaaactg |
| | | 1801 | taatagagtt ccggatcgcg gccggcgccg agttccttct ggtcatgcac aaaatcccat |
| | | 1861 | ttgtaatccg catggggcgt tgagtgcccc accttgccga gaataccggt aagatagccg |
| | | 1921 | gcatccctca gcgtctgcat gacagtcatc accaaggcgg cgcccaatcc tgcggccctt |
| | | 1981 | agaaaatcac gacgattcat cattgtcccc actaatcctt attgttcttc tcaagatacc |
| | | 2041 | ccgacaattt ctgcatttgc cgatacaggc cgccgggaca tatcagtata gccgcaaacc |
| | | 2101 | ttgaaaatat caacctcccg gaatataacg tcgacttcca acccagatcg ccaatccaga |
| | | 2161 | ataagaaaac aaagcaaaac gcttcaaatt cgtttaaccc cagggttcgc ctgaggttcg |
| | | 2221 | taaacaccat ctcgatgtac atcgggattc aaattcgttg agccccagcc cttcttgtgg |
| | | 2281 | ctcttgttcg gcaagaaacg ctgtaatcac cccacaaatc gggggctgct ccagcatcgc |
| | | 2341 | caagacgggc aatgccgctt tgaggctgta atcaccccac aaatcggggg ctgctccagc |
| | | 2401 | tgatttcgag tttcgatgct ttcggacagg gctgtaatca ccccacaaat cgggggctgc |
| | | 2461 | tccagcactc cttatggaga aggagcttat cgtgtcgctg taatcacccc acaaatcggg |
| | | 2521 | ggctgctcca gcttattcct tccatcatcc cgacagcagt gggctgtaat caccccacaa |
| | | 2581 | atcgggggct gctccagccc actttcgtaa ccattttact cgcaaacgct tataacgaaa |
| | | 2641 | acactttcca aaaaccatac caacgtcctc atttaacagg aaacttccac tccttttcaa |
| | | 2701 | ttccatattt cttcataaca tcactaaaca acccaaattc atctatcaca aactttaaat |
| | | 2761 | gatgatggaa aaacgctcta cgcaccttat tcacggcggt cttctccgcc tctttacaca |
| | | 2821 | ttgtttgtgc cagtatctca cgaaaatcaa tataatgcgc cccttccttc tcgctcatct |
| | | 2881 | ttttggcttt gacaaccttc tcttcaaacg ccagcaccgc ctcgacacat ttcttctgca |
| | | 2941 | gatcattata tgccctaaac cctttttcgt aaactgtatg ataccgtatc ttcccttttt |
| | | 3001 | cgtgcggcat aaagtactca catatccgcc caagaaactc agcgtcatcc aacacatata |
| | | 3061 | acttgccgta atcactcact gagaagacga tgcttttttc gttacccact gagccctcca |
| | | 3121 | cgggcaactc gatgctatca ttcgaccaca caattttatt acccaattcc ttgcgtacac |
| | | 3181 | tccccaggaa gtattgcgcc atcatcagac acaaacggtc tcgcgccagc gtttcataca |
| | | 3241 | aggctggatt agcaccctcg aatcgcccaa tcgcatcaat atgataatac tttttatcca |
| | | 3301 | gcccaacgtc cctctgtccg ccgccgcttt ccaaatgctc ttccacaagc ttcgcgaatc |
| | | 3361 | ccttcttgct gtcataccag aacttcttgc gcaagaaacc cctgcggata gaaatatgct |
| | | 3421 | ccttgaacca tgcaaccttc tgcttgtaat ctatttcatc cttcttccca agccccacat |
| | | 3481 | aatcgtagag cttctgatcg ccgattcggc aaagtctgtt gagaatctga tcacacacca |
| | | 3541 | cctgatgcat ctcgtttatt gtggaggtct gcgcaaaaat tgaatatacc cgcccgtcga |
| | | 3601 | ttcgctcggc cagttgcagg cgtttcagtc ccgcctgaaa attctcaaca tccttgccaa |
| | | 3661 | ccagacacac cagcagccgg ttgtactcgc cgggattgaa agacctcgtg caattttcat |
| | | 3721 | ttacgtattg aagaatgtcc cgcgccttct cgtgaagtgt catctcgttg gtcgccgcct |
| | | 3781 | tcttcttttc ccacacccct cgaacatgct ttactctgcc gtctattctt tgcttaaaag |
| | | 3841 | ctttcctgcc aatcccatgt tgctccagca caaatcgcgg caggaagacg tgattatcct |
| | | 3901 | tatctgtgac cttcactaca tccagaatgt tctccacatg ctgccgatac ctgtacagtt |
| | | 3961 | ttgcaatcgc atcgtcgccc tttccctgaa ggctaagcaa tacaaggtat ttcaattcgt |
| | | 4021 | taagccccat gcgataactc cgaggcccgg cattcttatc aatcctgacg ataacattgt |
| | | 4081 | tcttactgat atagtatgac tgatcttcgt ctttttttga aaagtcgaca actaccttgc |
| | | 4141 | ctttggtcct gtgctttttg tgttcgtcgc ctgccccggc ctcctccctg acaatgtgtc |
| | | 4201 | gccgcccgaa gcatatctca ctgtgttgcg cctccagata atgcagtgca aactcgatga |
| | | 4261 | acttgtcttt atggcgtttc ggattcgtcc cctcattgtc gtttgctctt ttcttgtcgc |
| | | 4321 | cctgctctcc gtggtagtat tcatacgcct ccgcagggat gcgtccaagc tgcgcgagta |
| | | 4381 | tatccctgaa aagcagcacg cgtttgtccc acgccttcgt gaaacgactg tctttcaggc |
| | | 4441 | aatacatcga aagcgccttc cgagtcagct tgtactgtcc ttcgtttttc ttaagcccac |
| | | 4501 | ttaccgcacc gtacaaacga tccagcaccc gccgttcaac aaagaacgaa acgaaaaaca |
| | | 4561 | caacccccgc cgtagtgatc cggtcgcctt cgaacaggct gggaaactcg atgatcactt |
| | | 4621 | cagtttcgcg tctcctgcat tcaaagatcg cccgctcata cgccctttcc atgattgtcc |
| | | 4681 | gcaactcatc ttctgctgta aatgtcagac acccgggcga atgtcgatag tgggagaaat |
| | | 4741 | agtttcttaa cgcctcggcc ttcgcattgg ccgcttgtgt gctacacttg atcaaagcgc |
| | | 4801 | gtgtatcctc atcgtaccag tcgtgctttg aatacttttc atggcgtaac agcgactcga |
| | | 4861 | caaaaagccc gtcgttctta tctcgattca caagagcctt gttgaaggca atcgtaaaac |
| | | 4921 | accatttccg agcaccttga tattcatcga tagacaactc tctctttttc gaagtctgct |
| | | 4981 | ttgacacttg cgccattgag cacctcccat tccagatttt agtgcgatct ttacctcatg |
| | | 5041 | cctccacaac actcccagcg ccaaacgttg agcaaagcaa aatacgccgc aggcgggctc |
| | | 5101 | cgtcgaatcc gtaatcctaa tttctaactt cccaatcatc taaaccgccc gcaaccgatt |
| | | 5161 | tgtcaaccaa aaaccacatc aatccgcaga tggccgcaga taaccgcaga tattgcaact |
| | | 5221 | aatccaccca acccaaaacc tctgttccat ctgcgccctc tgcgaaatct gcggacagct |
| | | 5281 | ttttttttcg tgcccttcat gtcttcgtgg tgaatttcat ttaacatttg acaaatatca |
| | | 5341 | aacggcatgg tataatgcgt tgcgtattta aggacaaagc aacaccaaaa acagggggag |
| | | 5401 | taaaaaaccg tgtccatcca aaaagaatcg caggccgcag gcctgccacc tatgattaac |
| | | 5461 | ctcggtcttt cagccaagga tgctccccac acccaaacaa gcgaaacgaa ccgtgcgcca |
| | | 5521 | agctaagctg gtgcaattca gcaggtgtaa tcctgcccgg tcaaaggtta gccgcccggc |
| | | 5581 | cggaatgaac atgtacgtat aaggaggcaa caaat |

| | Relevant | ATCACCTTTGTCTCCTGGCCCTTGTTTGGGCCATTGCCCGTTTTATCGTCCGGTGCGGATTTCCTGCACCC | |
|---|---|---|---|
| | sequence | GCCCAGGCAAAGACAAACAGCCAACATCAAGACCCATACTCTCATGCCAAATCGTCCAGTCATTTGAAT | |
| | SEQ ID | CCCTCTCAAAATAACTTTGCCCCTGATAATCGTTTCGTCCTGCTTATATGCCAAAGAAGGCCAAAAAGCA | |
| | NO: 5254 | AGTATCATAAAACCCGGAATAAGAAAATTTTTCGGCGGCTTCCCGCGGCCATGCCCCTAAAAGGACTTT | |
| | | CTACGTAAAGTGTATACTTACTGCGCAAAGAAAACCAGGCTCAGAGCCATCACGATCATGCCTATTATC | |
| | | AGACCATAAAGCGACGGGGGCGCTCACCCTGCGTTTCGGTACGCCTCAGGGGACGCTTGTCAGCTTGTA | |
| | | GAGTCGCAGCATACTCGGTGCGGGCAGCGGGCCTTTGACCGGACCGCGCGGATAGGCGTTCATCCTGCC | |
| | | CCGGCCGTCGGTCAGAGGCTTGTCCAGCAGCCGCCTCCAAGTCTGCGTCTCTGAATCGTAAACATTGTAT | |
| | | ATTTCAATACCGTTGCCGCTGCCGCCGTCGCGATAGCGGAATATGAACTCGTCGTCGGCCCCGCGAATA | |
| | | AACACCGGGTACGTACAGCGGTTCTCCTTTTCACCGGTCATGGCGTCTATTCGCTTGAAACTGGTCCCGT | |
| | | CATAAGGCCGCCCCGTCCTGAAATAGATCAACGGCGCACAATGCATATTCCCGGCAAGATGAACGTACC | |
| | | CCTTACTGTCGACCGCCATCTTGATGGAATTATGACTGTCCCAGCCGACCCTGGAATCCAGCTTTTGATA | |
| | | TTGCCATTTATCCGAATCCAGCCGCCGATAACAGCAGACAACATAACGTCGGTAAAACTCGTGAAATCT | |
| | | TCACCTCTTCGGTCTCCTTGAACCGGTCCGCACCCATTCTACAGGATCGACCTTGTAATAACCCTTCAAC | |
| | | TCATCATAAAGCTCAGGGTGCTTGCGTTGCATCCGCCTGGGCTTTTCAAAAAATGCTTCCGTTATGACGG | |
| | | CAAAGAACTCCGCCGGATTCGTCGCGCCGTACTTGTTCATGGCCGAGCGCCTGTGTTTTCGCGTTTTCGT | |
| | | GCAAAGCGCCTCGTATTCGGCACTCAAGACGCTCGCCCACGTGATATAGCTTGACCGGCTCTCGAGGAT | |
| | | AGGAGCGCCGTCGGCGGCGCCGTCTTCCTGGTCGAGCTGGTGAGAAAACTCGTGCATGACCACGTTCCT | |
| | | GCCGTCATGGATATTCCTCGCCCCGCCCATGACGCTGTCCCAGGCTAAAACAACAGGCCCGTTCCGCCA | |
| | | CGACTCGCCGAGCCGGACGCTCCGGCCCTCGATCACCATCACGCCGTCCCAGGATGTCTGCTTTGCAAC | |
| | | GTAGGTGTGAGGATAGACGTAGATGGTCTTGAGCTTCTTAAAAAACTTTGTCTTTCGATTCAGCAGCAGC | |
| | | ATGCACGCCTGCGCCGCGATTGTTACCCTGATTTCGTCGGTCATCTCCAGGCCCTGGCAGCCCTTGAAAG | |
| | | TCTTTTCCGCAACAAACACATTCACAAGCCCGCAAAGCTGCTCTTGCAAGTCGTCCGGCAGACGATTAT | |
| | | AAAGAGGGATGTTCTTTTCTATGTGCCCCTTCCAGTCATCGGGGAAAGGCGCACGCATCAACTTGCCTCG | |
| | | TCGCGCATCTCGAGCCCTGCGCCTTGCAGCCACAATCGCCACAAGCACAACAACTACCGCCGCCGATAT | |
| | | TATAATTGTCGGAAACATTCCAACTCCTTTGCCGAAATGGTGTACATTGCAATACCCTTTCACAAATGAT | |
| | | GCGAGGGATTCTGTCCGGTTCAGGCCCGCCGGTCAATGCACAATATACACTAATCGCCGCCCGGCGTCC | |
| | | ACCACCGAAGAGCCGCCGCCCCCTGTCGACTGGGCCGCTGCCACTAAGCCCGGTCAGGAGAAACTCCTG | |
| | | AGATTCTGAGAATTTCCCGGAGCAGGGCGGGACCGTGCAACTGTCTTTGGATCCTGAGCCGGTTATGAT | |
| | | AAAGGCGAAACAGCCCCATTATGGGACGCCACCGGCATGTCGGCCTGTGTGAGGGCGTATATACAAAC | |
| | | AACCGCCCTAAACAGCCCAAGTCGCAAGGTTATTGTTCCGA | |
| Cas13 | Locus | 1 | accggcggtt tttcatcgga ggtttggttt gctcaagcgg ctgcagattc aaattgaagg |
| b-t4 | sequence | 61 | cacggtgcaa ggggttgggg cccgtccttt tttttatcgc aaagccaggg acttgaacct |
| | SEQ ID | 121 | gagcggcttt gttctcaatg accgcagggg tctggtgctc gaagtgcagg gccggccgga |
| | NO: 5255 | 181 | taacctgcaa gccctgctga acctgctcga acatccgggc gggtgtgccg accggccgcc |
| | | 241 | tctgatgcag attgaaagct ggcgcgcgac accctgtccg ccgctggaaa acgaaacggc |
| | | 301 | ctttcagatt cttcccagcc gaacggaagg ggcgccggtg tgtcaaatca cccctgatac |
| | | 361 | ggccgtttgt ccgcagtgcc tgcgcgaact gttcgagccg tccgattttc gctatcgcta |
| | | 421 | cccctttatc acctgtacgc agtgcggccc ccgctatacc cttattaaaa gcatcccgta |
| | | 481 | cgaccgttcc aatacgacga tggactgctt tccgatgtgt ccgcgctgcc aaagccagta |
| | | 541 | caacgataag gccgatcggc gttttcatgc tcagccgctt gcctgtccgc agtgcgggcc |
| | | 601 | gtctctttcg ctgacggaca gtcagggccg gctccttgcc gatgaatccg acgccgccat |
| | | 661 | cgcccaggcc gcccgcattc tccgctccgg cggcatcgtc gctatcaagg gcatcggcgg |
| | | 721 | atttcatctg gcggcagatg cctccagcga agaggccgtt cagcggcttc ggcagcgcaa |
| | | 781 | gcatcgtcag gccaaaccct ttgccgtgat ggttcgctcg ctgaagcagg cccgtttgtg |
| | | 841 | tgccgagatt gacccgcagg ccgccgccgt gctggccggc ccgcaggccc ctattgtgct |
| | | 901 | gctgcccaaa aaagaaccca acccgctggc gccttccatc gccgaaggca ccaacacttt |
| | | 961 | cgggctgatg cttccttata ctccccttca tcatctgctg tttgccgagg agggcatctt |
| | | 1021 | atggctggtg atgaccagtg ccaacttcag cgaagagccc ctgctgtatg acaatgagca |
| | | 1081 | ggccctggcc gaactgtccg gcgttgccga tgcctttctg cttcacaatc gggatatcta |
| | | 1141 | tcggcccatc gacgattccg tccttcactg ggttgacggc gggccggcct ttctccggcg |
| | | 1201 | ggcacgcgga tatgtgccgg cgcccatccg aagaagccgc cccgtcctca aagaaatctt |
| | | 1261 | cgccgccggt gccgacttaa aaaacacctt ctgctttgcc aagcgtgatc aatatgtgct |
| | | 1321 | cagcgaacat atcggcgacc tggccgaagg caaatcgttc cgccattacc tttgggccgt |
| | | 1381 | cggccatctc cgctctcttc tggaggcgga accaaaggcg gtcgtctgcg accttcatcc |
| | | 1441 | ggattatctg tctgtgcggt ttgcccgctc gctgccggct gaacatttct ttcaggttca |
| | | 1501 | gcatcactgg gcccatatcg cctcagtgct ggctgaatac caactcgaaa ttgaggaatc |
| | | 1561 | cgtcatcggg ctggccgccg acgggaccgg cttcggcacg gatggagcca tctggggctg |
| | | 1621 | tgagtgtctg attgcctcgc aggttcaatt cgagcggttc gctcatctgt cgtattatcc |
| | | 1681 | tttggccggc ggcgatgcgg ccgcccgaga agccgtccgt ccgctgctgg ggctgctggg |
| | | 1741 | ttcgcagatt cctgcctcgc ttgaagcggt tcttgaacgg ctggagccgg accggaagaa |
| | | 1801 | actcgaaatc ctccgcctcc aggttcaaaa aggcatttcc gctgtgccga cgtcgagttt |
| | | 1861 | gggccgtttg ttcgatgccg ccgcggccct cgccggcctg gggacggtca acacgtttga |
| | | 1921 | agctcagctg ccgatggcgc ttgaggcggc cgccgaccct gcagaaaagg gactttacac |
| | | 1981 | cattcagata gacagccggc cgccgcagcc gctgcggtgg gagccgcgtc cgctcctgct |
| | | 2041 | ggagatggct gaggagttgg gccgacatgt tcctgctgcc gttgtgtgcg cccgctttca |
| | | 2101 | caataccgtc gccggcgctt tgctgcagat ggccgagcac gcccgccgtc aaacgggcat |
| | | 2161 | ccgcaaagtc gccctcgccg gcggggtctt ctgcaaccgc tttttaacca accggctgat |
| | | 2221 | tgaggccttg aaacaaaatg attttgctgt actctggatg cgcaaggtgc cggccaatga |
| | | 2281 | cggcggtctg gctctgggac aggccgccat tgccgccgcc cggatggaaa tggaacagaa |
| | | 2341 | aggttcacga taagaaaaac gatgtgttta gccgtaccag ccagaatcgt tgaactcgaa |
| | | 2401 | ggcgaccagg ccgtcgccga tgccatgggc actcgatgga ctatccgaac caccctcacg |
| | | 2461 | ccggagattc agttgggcga tatcgtcctt gtccacgccg gctatgccat caccaaaatt |
| | | 2521 | gacgaacagg aagcccggca gacctggcag ctctttgaag aaatcgcccg tctggacgag |
| | | 2581 | ccgccgcaga atcctccttc gtcgaggccg caatgaagcc cgcctgcatc gaacttcttc |
| | | 2641 | aagaccggat gtcgcaggcc gttcgccaaa tcggcaggcc cattcaaatc atggaagtct |
| | | 2701 | gcggcaccca tacggtggcg ctctttcggc acggcatccg ctcgctgctg ccggaaggca |
| | | 2761 | ttcgtcttct ttccgggccg ggctgtccgg tctgcgtaac cgaccagggc actatcgatg |
| | | 2821 | ccctcatcga actggcctgc cgagacgata cgctgattgc cacctacggc gatatgattc |
| | | 2881 | gcgtgccggg ctcaaagact tctcttgaaa aactccaccg ctccaacatc cggctggtcc |
| | | 2941 | tgagtaccga cgatgccctt caggccgccc gccggcatcc tgacaaaatc gtcgtctttg |
| | | 3001 | cggcggtagg ttttgagacg accgcaccgg ccaccgccgt cgctctccgc caggccctgc |
| | | 3061 | aggaagggct ggacaacttc accatcctct gcgcccacaa actcgtcatt ccggcgatgc |
| | | 3121 | gggcacttct ggaaggacgc aacgacaaaa ttgatgcctt tctctgtccg ggccatgtga |
| | | 3181 | gcgtcatcat cggctccgat gcatatcggc ccatcgcgga agagtttcac cgtccctgcg |
| | | 3241 | ttgccgccgg ttttgaaccg ctccagattc ttgaaggcat ctgccgcatc tgcgagcaaa |
| | | 3301 | tccagtcagg ccaggccaag gcagagtcgg tgtatcctgc cgtccggccg gccggcaatc |
| | | 3361 | cggttgccct ggcgatgctg gagcgtttct ttgaaccgtg ggacgggccg tggcgcgggc |
| | | 3421 | tgggctgcat ccccggcggt acgctccggc tgaagaaaga atatgaatcg ttcgatgtcc |
| | | 3481 | tccaacggct gaaccgaaca atcccgccat cgcctgagcc ggccggctgc ctttgcggcc |
| | | 3541 | aaatcctctg cgggctggcg gaaccggatg cctgtccttt cttcgccgga caatgcaccc |
| | | 3601 | ccgacaatcc catcggcccc tgcatggtct ccagcgaagg aacctgctcc gcctggtaca |
| | | 3661 | aatacaaggg ccggtaattc cattgcctgg gcccggggcc ttcttttccc ttccaagccg |
| | | 3721 | cttaacgttt tcaattctta aagtttccag cgtcccctgc cctcatcgcc gcgcaggcgc |
| | | 3781 | gatgcggaaa atcccctgcc cattccacgg cagtatccag aaccccccac tcaaaaaccg |
| | | 3841 | cacctaaaaa tccgactctg cccttcaacc ctcccaaaat ctgaccttac ccaaaaatgg |
| | | 3901 | ttgtgattac cccacaaaac gggggctgct ccaactgtta tcgacgtgta cggagcggtc |
| | | 3961 | aatgtgttgt gattacccca caaaacgggg gctactccaa ccaggatttg ttcgtcacgt |
| | | 4021 | attttgcacc ggttgtgatt accccacaaa acgggggctg ctccaacgag gcaagtgttg |
| | | 4081 | cagtctgggc actctatgtt gtgattaccc cacaaaacgg gggctgctcc aaccttcgag |
| | | 4141 | aaggcagaca cacgtcggtg caagttgtga ttaccccaca aaacgggggc tgctccaaca |
| | | 4201 | tcggtgagca ttgatgtcat aggtatgatg ttgtgattac cccacaaaac gggggctgct |
| | | 4261 | ccaacccccg ttctgcaagg tacgagttct aaacaagtta tacccatttc tttcggcaaa |
| | | 4321 | aacaaggggg aaaccacgtt ttatctcttc tttttcttcg gtttttctct tttttcctta |
| | | 4381 | ttcagatatt ccgacattcg tctttcaaaa tcccgccatt gagaaggtgt cgcctcaaac |
| | | 4441 | tcttcatgga aaaaatcatt ccttactcgt cttatactct ctttctcctc ttcgctcagg |
| | | 4501 | cggttttttc cggaaaatcg ctcaagaacc tcatcaaaag aaagatggct ttttcccttc |
| | | 4561 | cattcctctt cagggatctt caggttttct tcaaagcgga acagggccct gaccaccaat |
| | | 4621 | atctgacgct gcctgtaccg ctggaggccg tcccgaagaa actccggcca ggttatctct |
| | | 4681 | tttcttccgc tgagaaacca ttgacagata ttgtcaagca tcggcgcctt gttcagaaag |
| | | 4741 | tccagtctcc caaaatcctg cacccggaag ataatcctgc agccctgagg ccccttgtac |
| | | 4801 | tccactttga agcgattgta tccgtcggat tctttgcttt cgctgttgat ttcccagtca |
| | | 4861 | attttccctt ttacctgagg ggtcagatgt tcatagtgcc attgagccat ttgcatgcac |
| | | 4921 | agcaagtcct gcacataaag ataatacatc ttttggatga ttttccggtc ggaccatgga |
| | | 4981 | taatcgccgg gctgctcttc cctcagatac aaacgctcct catggaaaac agaagttttc |
| | | 5041 | ggtttcaggt gttcctgaac caacttcaag agattcgtcc gacggctgcc gccggttaca |
| | | 5101 | aaaagtttgc agcgaaggaa gtatttgggg acagagagct ggttttcgat aaaccgttca |
| | | 5161 | aacttcctgg cgggagtgtt ctgttcttcg tagtgtttcc ctttttcctt cggcagcagc |
| | | 5221 | ccgatataac gtttgagttc gtcgccgccc tgcttttcaa gttcgtccag acgctgctgc |
| | | 5281 | tgaagctgac agcatcgttc gcacagttcg ttgatgcttt tgcattgggc cagattattc |
| | | 5341 | cagactgccg cgtccagttt tctttcggtt tggaaacttg ccagtttgcg atagaaatca |
| | | 5401 | tcaaaagcca gcgtctggag caattcccgc agttcattat actccttcac agagggacgt |
| | | 5461 | ctcctgaagt ccggcatgct gtcggaaata aatctcagga tatgacgaat cttttgtcct |
| | | 5521 | ttgtccagaa tccattgtcc cgtttgcgct tccttctgaa ggttcgcctg tatctccttc |
| | | 5581 | agcgtctttc ggatatagtt gagccgattc tgcaccgcct gctcagaaac ctccgaagcc |
| | | 5641 | ggcgacctca aaaaggcagg aaagtttcgg ggcatgttcg ttaacggccc gcggcttcgc |
| | | 5701 | attctgacat cccaaacata cttgctcagt ttctggacgg cattccctcc tttcccgctc |
| | | 5761 | aggattgcaa gaacaaggta catcaattca tgctcggaga tacgaaccgt aacgggggtt |
| | | 5821 | ttggctttaa gatgaatgcg gatgagggca tggttattcc gaatataaag aagcccttcc |
| | | 5881 | aaatcctgtt cttccgctga aaggttgttt aaagcagaag ggaaaaaaac ttctctgacc |
| | | 5941 | tttttgccgc tctgctttct gcggtcttcc tcgttttgaa tgtttctgta ccgtccaaac |
| | | 6001 | tcgacgctga gtttttcatt cctggcccat gcttcgataa accgtacagc gaaaagaata |
| | | 6061 | aacttgtttt ttttgcgcat tctccggccg gctggagcgg tctgcttctc cgtctgggca |
| | | 6121 | gggttttctt cgtcctgctc ttccacaggc cggccgaaaa attctttctc ttctgacttg |
| | | 6181 | ctcagttttc catgggcttg aagccactgg acagaatcga aaggaacaca cgacaaatag |
| | | 6241 | gagaggattt cccgaaaggg ccgtgtctca tcggcttgca gataataact gtctctgagg |
| | | 6301 | gtatagaaac ttagaagaga acgggtaaaa taataatccc gcttttgccc ggtatccaaa |
| | | 6361 | tcgccgcgat cgctccgctt gaagccctgg accgctccga acattcgttc cagcgtgctt |
| | | 6421 | cggggcacaa aaaaggacgc cagaaaaata acccctgccg ccgtaatccg gacctgatcg |
| | | 6481 | ccgcagagtt caaaaatggg agggataatc ccctgatatc cgctttcctt aactcttttc |
| | | 6541 | tgagtttctc ctgcggcata cgaaaacgtt ttttccagaa agtcctttac tgcgtcatcg |
| | | 6601 | tctcctttct tgaactccag tggtttatct tcgtgataat agtgagaaaa ataatcacga |
| | | 6661 | atgccgttca gtttttccgc cagagaccga tgaactttct cgatatcctc ctgggtaaag |
| | | 6721 | ccgcccctgg ttcgcatggc ctccttaaga agattctcac accctttttt cgaaccattg |
| | | 6781 | tcccaggcaa tattcagggg aacagtgaaa cagcttttgt gagtcccttt gtaataatcc |
| | | 6841 | tggtctaaag agtagttaac aggcatggta tccctccttc tttgccggct ggttttgggc |
| | | 6901 | tgttttctgc atactcgaca cggattcttt tacatctttc ttgattccct cgcaagcgga |
| | | 6961 | ttttatgccg ccgattttcg gccttccgat tttttttgaa atcgggaggg ggtgggcggc |
| | | 7021 | gtcttactgt ttgttcaaac gtatagaagc agagaaaggg actatattca tattatagaa |
| | | 7081 | gaggggatat attatgaata tttcactgga aaccgattcc gactcgtcgc cgctggggtt |
| | | 7141 | gcttgtccgt tatcggcagc ttttgctttt ttatgccctg gccctgtttt ttgacacggt |
| | | 7201 | cagcaccatc cactttatga atcggggcgg catccacctc gaaattcacc cgctggttcg |
| | | 7261 | ctggggcgcc cttatctacg ggcctatcgc ggggccgttt ctgtttgcct ttcttttcaa |
| | | 7321 | gtttttgtcg gggctgctgg tgctgtttta cgtccgccgg cttgctcctt gcattttgcg |
| | | 7381 | gcttgcggcg gcggtttcaa cgatagccgg catcctgaac ttctggggcg agtcgcttct |
| | | 7441 | gatgcactaa aaaatcgccc gccgctcctg tttttcagcc gcacaagacc gataatatcg |
| | | 7501 | cctttccccc gattaagccc gagccggcgg ccaaatccag ccggtttttg ccgcggccgg |
| | | 7561 | ccgacggcgg gcttctcctg cggccggcaa aggggtataa tagctcatag ggaaaaactc |
| | | 7621 | ttgaaaggtc ccgtccaaat gaacccatgg atgcaagccg ttcttgtctt atcggcggcg |
| | | 7681 | tcg |

| | Relevant | AGACAAAAAGGGCTGGGGAGGGGGTTTGGGGGAAATTGCTGAGAATCTAATTGGGGCACAGATTCTTG | |
|---|---|---|---|
| | sequence | AACCAGAGGGGAAGGGGTGAGGGGTTGCGGGGGCAATTGGTAAGAATCCAATGGAGAGGCGAGGTTTT | |
| | SEQ ID | GAGCTGGTTGGGAATGACTGCGGAAAGGGGTTGGGGGAGACGGCTGATAATTCAATGGAGAGGCGGAT | |
| | NO: 5256 | TGGGGGGCCAGGTGGAAATGGCTGAGAGGGGGCAATGGAGAGGAAGTGGAGGGGGAAGATCTGAAAT | |
| | | AATGGGGCAAATAGAGGGGGATTGGGGCGGATGAGGGGTGTCGGGTTGTGCTCCTTCTGCTGCCCATTT | |
| | | TGATTTCTTTAACTCCTTGCGGCTCCTTGGGGCCGAATGTCTGCTCCTTCGTCAGTACAGGGGGTATTATA | |
| | | CCATATATGATTTTGAAATCAAGGAAATGTTTGGTAATTATCAAACTTTTTTTGCGTGCAGGCGGAGCCG | |
| | | CATTTTAAGAGTGAAAAGTGAAGAGGGAAAAGGGGAATAGTCGAGGAAGCGGGGCTGGTTTGGGGTTT | |
| | | TGGCAAAAAATAAGCAGAAAATGGGAGAATGGCTTGCCGGAGGGGGTTGGCGCGGGGTATAATGAGGG | |
| | | GCGGTATGGCTGCTTTGAAAAATATTGAAAAATCGGAAGGAAATAAAAATAAGGAGTTTGCTAAGGAG | |
| | | TCTGCCAACCTTTGGACGTGCACCGAGTTTCTGGGGCTGAGGAGGGAATGGCTCTCGAAACTGAAATTT | |
| | | TTAGAGGTGCCCTTAAGGGATTTTTGCTAATTCCTTATAAAAAGTGGAGAACAACTCAGATATTGTCGAT | |
| | | AGAAAAACAAGGGAATCCCCAAAAATTCTAAGGAAAATCTGTCAAGCTGTAGAAAAATAAGGTTACTT | |
| | | TGGATATATTCAGCCCTAACCTTTGGGCTTGCTTTTTATAGAAAAACATACTAAAATAAAACAAAAATA | |
| | | AAGGATTACCTGTTTTGGCAAAACAAAAAAACCCCAAAAAAGCAGGAGTACAGAAGGCTGAACCAATA | |
| | | GTCTTATCTATTAATATATGTGATATAATCATTAGGGATGAAAAAACCAAGAAGGTCTCCCTCATAGGTT | |
| | | TGTTTAGTAACATAAATGCATATGCTTTTCCTGTTCAGCATCCTTTGATGCATGTATATATCGCATTAACA | |
| | | AATGGCCATGGAAAATATAAAATAAATATCCAATTTGTTAGAGTTGCGGACAACAATATCATTGTTGAA | |
| | | ATGGAAGGCGAAATAGACTTTCCTGATCCGTTAGGTGTTGTAGAAATGAATCTTGAGTGGCGAGGAATA | |
| | | CAATTCGAGAAACCAGGAACATACTCAGTTGAAGTTCTATGTGAAGGTTCACCAATTGGTTCTCGCAAA | |
| | | TTTAATGTAAGGCAATTACAGAAACAAATACCGCCTACCAAAGGAACTGAAGGAACATAATATGTTAAA | |
| | | TGACTCTAACAACTTTGTTTGTTTTCCCTCGCAGGAACGATATTATGATTTCTCTGCCTGTCAATTCGTAC | |
| | | CGTCTCACTATATATGCTTGCGCTGGGATAGGGCCGAAGCAGAGGCCACAGAGGAGGAAATTGCAGATT | |
| | | ATGCAGCTAAGAGTCCGGCTTTCGCTTTTCTTTCTGATCCCGAAGAGGATATCTATAATTGTAATGACGG | |
| | | CAAGCCGTTATGAGTTTAAAAAGAGGGGATATAGTACTGCTTTCTTTCCCATGGACGGATTTTACAAGTT | |
| | | ATAAATTTCGTCCGGCACTTGTCATTTCGGAAGACGATTTTAACAAGAAAAACAATGATGCAGTCTTTAT | |
| | | GTTCATAACTTCGAAAAGATACTCTTCTGATTACGATTTTTACCTTGATTCAGAAGATCCCAGTTTTAAC | |
| | | AAGACCGGCCTGAAGAAATCCTCTACTTTCAGAATCTCTAAAATTATTACATTAGAACAGAAGTTGGCG | |
| | | AAAAGACACCTGGGCAATCTCGATAAAAAAGTTTTGAAAAAACTGGAAGCAGGTTTGAAGTTACTTCTA | |
| | | AATATATAAACTTTCGTATTTTTATTAACTGTATAACTAAGCTTCTTTCGCAAACATTTTTACAGAACTTG | |
| | | CTGAAAGTGTCAAGGGGGCGTTTTTCACGGTTTGGCAGATGGGGGTGGGGCTGCCGTCGTTTTTGATTTT | |
| | | CCGCTAACTCCAAACATTTCCAACAAGGCACACTGTTATTGCGGCGGCCGGGGCGGGGGCTGTTTTGAT | |
| | | GCGGTTTTGCCGGACGTGCTAAGGACGGTGTTAGGACGGTGCTAAGAAGGAAAAAAGAAGGAAAAGAG | |
| | | AGAGCGGGAATAAAAAAGCGGGGGCCAATAAGAACAGAAACAAAAAGGTCCTGTTCCGGGGTCGGTG | |
| | | GCCCCCGCATAGTATGCCTGCGGCTTTTCGTGTTCAGCGATTTTACCATTTCGAATATCCCCCATTCAAAT | |
| | | GATTCTTTTTATTTTTGAGTTGAGCCGAATTGATATTGATAGAGCATCCGTCTGGGAAAAACGTTCCTTTT | |
| | | TTTCAACCGGCGGGGGCCGCTGCCTTTCAGCGGCTGTCTTCACTTGAAATATTCGAAAAATCTTCATTGT | |
| | | TCTTACCCCCCATAGCAGGTTTCATCGTTAACTATGATAACGGGATTATACAAAAAACCTGCTTTGATTT | |
| | | CAAGGGTTTCTTAAGAAAATATTAAGATTTTTTAAGATTGTGCCAAAATGCCTTAATATTTTATTAAAAC | |
| | | AAGCACGTTATCGGACGATGGCGGCTGAACAAAATGAATTTTTTCGAGGGCGGCCGGTTTTTTTGATTGC | |
| | | AAAAAAACGGCTTTTTCCTTTTTACATAATA | |
| | Relevant | GCTGGGTTGGGATTGGGGTGGTTTTACTTCGCCCGCACCTACCGCCGAGCCGTGTGGGGGGCGGTGGCG | |
| | sequence | GCTGTCGGCTGGCTTGCGGTTGTCTTGACTGCTCATTTTGCCCATCTTTTCAGCACATTCGGCGGTCTGGA | |
| | SEQ ID | CTGGATTGCCGCCTCCCGAAACAAGTTTCTTTTTATCGGTTTTGCCCTCTGTTTCGGGCTGGTCGGCCCGC | |
| | NO: 5257 | TGCCTCACCTGAACCGCCGATGGAAGCAGGCCTTTACGCTTGCGGTACTGGCGATTTTTGCGGTCTTGTT | |
| | | CATAGGAATCCCCTTCGTTGGGCCTGCCGTGTTTGGGCATCAAATAAGCCGTCTGCCGACGCATCTGGAC | |
| | | CAAAACGGCATCTGCCGGCAATCCACTTCCTTTACCTGCGGGCCGGCGGCGGCCGTAAGCGTCCTGCAC | |
| | | CGGCTGGGGCTGGAGGCCTCCGAGGGGGAGATTGCCCGCGCCTCCGGTACTGCACCGGCTCTGGGTACG | |
| | | GGAATCTGGGACCTCTACCAGGGTCTGCGGCGGCTCTATCCGCCTGAGCAGCTTCAATGCTGTTATCTGC | |
| | | GTGCAGGGTCCCTCGCGCATTTGCCCGAGGGGGAGTTTATCCTTGCCGTCATCCGCGAAACATTCTGGCT | |
| | | GGATCATTGCGTGGCCATCCTCGAAATCAGGCCGACATCGGTTATTTTTGCTGACCCGGTCAACGGCCTG | |
| | | TCCATCCTGCCCCGAAGTGCCTTTGAGGCGTGCTGGCGAAAGTCGGCTATCGTCCTTCGCCGGCCCGATT | |
| | | TGCACACGGCCGGCCTTTGAAATTTTGCCTTTTTCCCTGCCGGCCGGCTCTTTGCAAGGGGCTGCTTGTTT | |
| | | GGTATAATCTCCGCTGACCATTTATGTTATGGGAGATTTGATTATGAAATTGCCGAAGATTGAAAATGCC | |
| | | CCCCGCTATGCCGGCCTGTATATCGTGGATTTTGGAGACCACTGCGGGGTCGGCTTCCTGGCCGAAGAG | |
| | | GTCGCCGAACTGCTCGAGAGCGAACGCTTCCAGCACATCACCGTTTACAAAATATACAATGCCTACCCT | |
| | | GATGGGACCCTCGAAATCATAGGAGTCCGTAAGGAAATCTTTCAGCAGGAAAGCGGGATGTTCTTTTAT | |
| | | GCCTTTGACGAGGAGACGGCTCGGGCGGATTTTGCCCGGCTTGCGGAATGTTCCCGTCGCCTGGAGCCG | |
| | | CCCTCACGTGCGAAAATCCAACTGGCGGAACTCGCCCCGCGGCAGTATGCAACCGCCCTGATTTATCCG | |
| | | GCCGAATATGATGCCGAATTCAGCCGCTGGCTGCTCGATTGCCGCTACCGAACTGAAGGCCCGGCCGCC | |
| | | GGCGGGGTCAGTGCGGTCGAACAGTATTACCGCCAAAAACCGGCCATTCTCGAAAGCACCCAGATTTTT | |
| | | AACAGTACCTCTGCAGCTTTCCACGGCGAAAATCTGCTGGAGGCGGCCAAAAAGGCGATGGTCCGATGA | |
| | | CCCGGACGAACCCCGCCAAAAAACGCCTTCTTGGGGCGATTGCCGGCCTTTTTATCGGAGCCAAAAAGC | |
| | | CCGCTGCGGACTCAGAATCCGACAAAATGGACCATTTCAAAAGCAGCACCCAGCGGATAGGGGTTCGA | |
| | | TTTTCTGAGAAAATCCGCTCCGTTTTCCGGAAAAGGTGGATTAAAATTCACTGAGGTTTTTCTCATTTTTC | |
| | | TCTTGACAACGTCCCATAAAATTCCTAATTTCTGGCATCTAAACGCCTTATTTGGCGGTTTTTTGCGGAA | |
| | | AACCGCCGATAGAGGACCGGGGAGTTTCGTTCGAGCAATTTTTTATTAAAGATTTCAGATATGCCTTGCC | |
| | | GAAACAGCAAGAGCAAGAGTTTTCTCCCCTCCGGAAAACATCAGGAAATTAGGGTTTCCTGATGTTTTC | |
| | | TTTTTTGCGCACCGGACCGGATTACGATTTGGGCAGAGTGTACTTGGAAATATCGCCGCCCTTTTCCTTG | |
| | | ATAGAGTCCAGATAGACATGGAGCCGCTCCGTCAGTTGGCTGATGGTGCCGGTCAGTTTTGTAATGTCAT | |
| | | CCTGAAGGGCCTTGGCCTTTTCGCCCATCTTCTCGGCCAGCGGAATCGCGGAAAATTGATCCATCAGTTT | |
| | | CTGGAGTTCGGACTGTTTGGCCGCGATTTCATCGACATACTTCTGGGCCGTCTTTTCCAGAGATGCGAGA | |
| | | TCCATTTTTGCCGCGGCCTCTTTCAGGGACTCGATGGAGGCAGACAGGTCGATGCCGGCTGTCGAACCG | |
| | | GCCCGGCCGCCGGATTCCTTTTTGCTGCAGCCGCCGGCGGCCATCAGCAGAATGGCTAACCCGGTAAGA | |
| | | ATCCAATTCTTCATACAACAACTCCTTTCTTTGAAATGTTGGTTCACCCGCCGTCGAAGGTTCGCAGCGG | |
| | | CGCCGTTTTGTTCAGCGTATCCAACCTCTTGTTTTTTCGCAAGATATTATTTCCCGCCCCGGCTTGCTTCA | |
| | | GAACAAGCAGAGTCCTCCAGGATCCTTGAGCCATTCCAACTTACACAGTTTATTTCACTTCCTCAGTTTC | |
| | | CAGGTTCCATCCTCTGCATAATTGGAACTATAGGGTAAGCCAAATGCATTCCATACTGAATCAAATATCG | |
| | | GCCTCATAAAAGACGGGACTTCTTCATTGTAACTGCCCACTTCTACTTCAGGAAAAATAACAACATCTTT | |
| | | TTCTATTGGAACATCAAATTGCATATGGGATTTTGTGTCTAAATAAGCACCTTTGCATCCGAGCAACGCC | |
| | | GCTGAAATTATAAAAGGAGCTTCTATGCCTAATTTTTTATATCCTTCAAAATACCTTTCGATAGACGTCA | |
| | | CTATGTCTCGTTCAAAATCTTGACTTGGCAGGAAATTTCTCAATCTCCCTGGATAAAGGATATCAGAACG | |
| | | GACCGCTTCTACAGCGCCATTAAAAAACACAAGACAATAGCTGCTCCTCCCCTCATGATCCCTGCCATA | |
| | | GGTTAAGAAGCCATCAAGATTATATCGATAACCGGAACAACTACCATTTAATGGTCTAAAAGAATCCAA | |
| | | AAATTTGTTATAAAATCTTAGGTCCAGTCGTTGACGGTTGAGAAATGGATACAGCGGTATAACATGCAG | |
| | | AATCAAGCGATCTTTTGACAGCGGTAGCGGTGTTTCATCAGCCATAATCGCGGCTAACCGATCCTGCAA | |
| | | AAAATTGCGGATTCGCTCTGCCTGCGACTCCGTAGCCAAAAAAGCACTTCTTATTTCATGAACATCCAGC | |
| | | GGAGATTTGCCTCCCGAATTTCGAGCATAGAAACGGGAGTGCCCTCCCAAAGTTACCATATGGGGTGAT | |
| | | GCAAAACTTTTGGGAATACGAACAAGAAGAACAAAACCTTTTCCGTCATCGCCAAAGCCATCGATTGTC | |
| | | TTGATTTGAACCCGAACGCGGGGTTCAATGCCGTTACGGATCAGATCTTCAAGACGCCGCTTTGCCTCAT | |
| | | CTGCGGTTATGTCTTGAAGAGGAACTACTGAATCAGGTTCGCCTGTTTTTTGGCTATTTTCTTCGAGTGTT | |
| | | CCTTCAGGTGTTTCTTCGACTGTTTCTTTAACCGCTTCTTTAATACCGTATATAATATCGCCGCCCGAGGC | |
| | | GTTCGCAAATGAAGAGATATCTGCCAGAAATTTCTTCTCATCAGAGTCTCGACGGCCGGGAAGTTTTAAT | |
| | | TTGTATTCAAGGGTCTTGCTTTCGGCGATTTTGTTTTCTATCAGCCAATCAATGTCTTCTTTTGTTATTTCA | |
| | | TCAAAATTTTTCTGTATCATGGTACCTCCTTTTGCTGTTTTTCCGGTATCCTTTTTCCTTCGTTCGTCTCCT | |
| | | GTGGGCCGGAGGGTTTATAATGGCTCCGGCTGGTTTAGTTTATCTTCTGTTTGGCTTTTTTGCAAGATGCG | |
| | | GGGGTTCCAGTGCTGATTTTGGCGAGGAATTGTCCTTTTTTCAGTTTTCAAACACCGAACCTGGACTGCC | |
| | | GGCGGAAAAAGCCCGACGAAAGGAGATTAGACGGGTCGGGAAATCGACGGCAAGGGGCAAGGCATCA | |
| | | GGATTTTTCTTCAGCGGTCCCCAGGGAAATGGGAGAATAAAGAGGTGTTGTGTCCTGAACCGGCCGGCG | |
| | | GCAAACTCTTTTTCCAATAAATTTTTAAAAAAATCAAAATTTCTCTTGCAAATCGAAGGGAATGCCGATA | |
| | | TATTACCTTGTGGATTGATGCTCTTTTTCTATACCCTATTCAATGGGTCAGGACGCAGGTTGATAACCTTG | |
| | | GTTCTATTTAATTTAGGATTGTGCGTCTGGCCCAAGGCAGATATGTCTCTCATATCTGCCTTTTTTTTGCG | |
| | | CCCTGCGTGTTTGCCCCCGTTCAGACTGTCTGTCTTCCGTATTCCGTCAGATAAACCGCCATCAGAATAT | |
| | | GCCGCAGCCGCTCATCGGTCTGGCCTTCCAGGTAGTCTTCGGTAAAGTCCAGTTTTAACCGCCCCTGAAA | |
| | | ATGGAGCAGGCGCTCCTTTAATTCGCAGCGGCTCATCGAGGCAATCTGAACGCTTATGGCTTCATAATTA | |
| | | TTCTCAAAACTCATGGCTTCCTCCAGTACAATTTCGCAAGCAGGTACGCCTGTTGTATCGTTCCTTTCGG | |
| | | GCCGGCGGTTTATGTGCCGCCGGACAAATTCGCCGCTTCAGCCCGCGGGGGCGCAGGGCCGGCCGCTAT | |
| | | AGACGCTACAGCCCTTTTAAAGGAAAATATCATTGTCTCCGCCCGTCGGCGGTGATAAGGTTTCTGCCCT | |
| | | ATGAGAACAGACCAACTGGAGTATGACCTGCCGGAAGAATTGATTGCTCAGCATCCGGCCCAACGGCGT | |
| | | CCGCAGTCGCGGCTGCTGGTGCTTCACCGCGCCGACGGCCGGCTCGAAGACCGCCATTTTTGCGACCTG | |
| | | CCTGAGTATCTCCAGCCGGGCGATTGTCTGGTTCTCAACGATACCAAAGTCCTGCCGGCCCGCTTTTTCT | |
| | | GCCGAAAACAAACCGGTGCCAGGATAGAGGGGCTTTTTCTTGAAGAAACACCGGACGGCAGCTGGAAG | |
| | | GTCCTGCTGAAAAACGCACGCAAACTTAAACCCGGCAGCCGACTGACATTTCTCGATCGGCAGGGACGG | |
| | | CAATGGGAAACGCTGCAAATTCAGGAAAAACTCGAAGAGGGGCAATGGCTCCTCCGGCCCGACACGCA | |
| | | GAAACCGCCGCAGGCCATCCTCGAACACATCGGCTCAGCTCCCCTGCCCCCTTATATCCGGCGTCCGGC | |
| | | CTTTCGGCAGGAGCCGCAGGAGGATTTGGCCCGCTATCAAACCGTTTATGCTTGCCGGCCGGGCGCCGT | |
| | | GGCCGCCCCGACGGCCGGGCTGCACTTTGACAGACCCCTTCTTCAGCAGTTGGAGCAGAAGGGCATCCG | |
| | | CATCGCCTGCCTGACCCTGCATGTAGGAATCGGCACCTTTCGGCCTGTGCAGACCGAAACGCTCGAGGA | |
| | | ACACACCATGCACAGCGAACGATACGAAATCAGCCCGCAGGCGGCTCAACAGATCAATGCCGCCGCTG | |
| | | AAGAAGGCCGACGAATTATCGCCGTGGGCACAACCTGCGTGCGCACCCTTGAGACCGTCGCAGAAAAT | |
| | | CGAAGAGTCCGGCCGGCCGCCGGCCAAACTGCCCTCTTCATCCAGCCGGGCTATTCATTCAAAATCGTG | |
| | | GACGCCCTCATTACAAATTTTCACCTGCCCCGCTCCACGCTGCTGGCTTTGACGGCCGCCTTCGCCGGAT | |
| | | TGGAAACCATCCTCAGCGCCTACCGCCGCGCCGTCCAATTGCGATACCGCTTCTACTCCTACGGCGACGC | |
| | | CATGCTCATCCTTTAAACCCCTGCAAGGCAACCCCCTCTAAAGTTAATCAAAAAGGGACTGTTTACCCTT | |
| | | GGATGCCGCACTTTTATTAGTGGTCCCATCAATCACTTTTAGTAATGACCAGTACCCACGGCCTGATTTT | |
| | | TCAGTTAAAATAGCAAGGGCATCTCTCTTTTTATTCCCTTCTCTCCGAAGCGATTCGTATTCACGGCGCA | |
| | | AAAAATCTCTTTCATGAGCGGTGATTGGTCTGCCTTGATTATAATATGTGTTTGGCGGCGGTGTGCGCAC | |
| | | TTCGGACAGCAATGAACCTGGAATTATTTTCTCCATTTGCTTGATTTGATAATCGAAAGCAACTTTACTC | |
| | | TTCTCAAAACCTGCGGCCTTTCTATTCAAACCTATGGCAACTTTAGCCGTGGAAAAACTGCCCAAAAAA | |
| | | AAGTCGCAAACCAACTCCCCCGGATTGCTTGAATACTGGATCATCTTGATGAGAAGCTGGGTGGGAAGC | |
| | | TCGTTTTTGTTTTTTACTTTTCCCGGCTTGTATTCTCGATTAATAATCCAAACATCCTCACGGTCCTTATAG | |
| | | TTGAGCGCTCCCTCTTCATCCCTTTCGCTGTCCG | |
| Cas13 | Locus | 1 | ccccccatcg aacgctgtga ttaccctgca aatcgagggc tgctccagca taagtgctgg |
| b-t5 | sequence | 61 | tatccgtaca cgggacggtg ctgtgattac cctgcaaatc gagggctgct ccagccgcca |
| | SEQ ID | 121 | agattaacgg acaggatgtc tactggctgt gattaccctg caaatcgagg gctgctccag |
| | NO: 5258 | 181 | ccgctctatc gcaaggtatt atctcaattc catttatggc aatatttaac agtaaatttc |
| | | 241 | tgtatagagc aatcactttt gctttttatc tcgccgtttt tttgtgtcgt cttttcgttc |
| | | 301 | gacaatcgga aagtatttct gaaaaatcgc ctgctgatct tctgttgact taaattcctc |
| | | 361 | atgaaaaaaa tcatttctgc cgcgattcag agcctccttg tctttgcctg tatcttttat |
| | | 421 | tttaccttgt tttaccgcct cgtcaagaat attgcaaaat ggaaagtatc tctcatgact |
| | | 481 | ccatttatcc tcttgtattt ttaaaccttc ctcaaacgca aatatcgcac gcacagcctc |
| | | 541 | ggcctgccgc tttctgtatt ttgcaatgcc gtctttattg aatttatccc aggttatctt |
| | | 601 | cttttcggca cccgaattga gttttttgtt tccgagcaca aaacactggc aaatattttc |
| | | 661 | tatcatcgca ggtttttcaa tgatatccag tcgcaaaaaa tccgccggcg taaattgtat |
| | | 721 | gacaaccccg cagcctgtct tataccacag cgaatatcgc tcataaccca tatttccctg |
| | | 781 | cccaatacaa ttccattcca atttattcct gaattcagtc gcggagatta atttctcata |
| | | 841 | atgccacatc gccatcttca tacagagaac atcttgtata taggtgttgc acatctgtcg |
| | | 901 | gataatcctg ctgtgtttat tgtcatagtt cttcggattc ctgtcgataa gataccaccg |
| | | 961 | gtcatcgtgt atgggcttga tgtccttgag tttttctttc acaatgctat acagactttt |
| | | 1021 | tcgctctttc gcttgtgtat tgtaaccttt ttcttcgtca aagcaccgct tttgacaaca |
| | | 1081 | atcgccataa aacattttcc gcaaaaagtt tttcccgata gaaaactgcg actccagaaa |
| | | 1141 | tcgcttggcc ttttcctgca gtgtgttcca ctctccgtac ttcggatgtt tttcctgcgc |
| | | 1201 | ttcaagccca atataacgcc tcagctcgga gccattcttt gcctccagcc cagccagtct |
| | | 1261 | ttcaagtgtc agttcgcaga cttttttaca aagcccgctg atgtcattga cacccttgat |
| | | 1321 | ctgatcgtat aatgaaaccg caatccggcc gtcattgacg taattttgaa gttccttata |
| | | 1381 | aaatccatca aagtcgagcc tctgaagcgc atcgcgtaaa atatgatatt gtttcacatt |
| | | 1441 | cggccgcctt tgaatatcgc gaatgctgtc ggagataaac ttcagtatca ttgatatttt |
| | | 1501 | tttgcctttg tatatcagcc acttattatt ccgcggctct tcctgaccga ttttctctat |
| | | 1561 | cgtctcctga agttgtttgc gaatatagct taaccggctc tgcactgcct ccggcgtaac |
| | | 1621 | cgtcctgtcg gcacgcttgg taaacgacgg catccatcgc tcatcctccg gcctgtgttt |
| | | 1681 | ccattcgccc tcgatttttt ggctcatact gtaaatataa caattcagct tttggacggc |
| | | 1741 | gtcattgccc ttctcctcaa aaatcagcag caccagatat tttaattcat tttcagaaat |
| | | 1801 | acgggcagca acggcttttt tattattcag tttaatctgg ataattgcgt gttcattctg |
| | | 1861 | aatgtaatac atccattcgt tattgaaatc cggattgagt ccttggtttc tgtattttaa |
| | | 1921 | ttgcaccgct ctggcctgtt tgccatcctg attcttattc tccgcttttt ccactgtttt |
| | | 1981 | ttggtagcgt gcaaatgtca catctaatcc ctgttcagcc gcccacccct caataaattg |
| | | 2041 | gatagcaaat aaaatgaatt tttcatctct gcggagacta cgcctctttt ctttgctttt |
| | | 2101 | tttattttta tcactggacg aagagtccga aatatccccg ccctcatctt cggcgggttg |
| | | 2161 | attcaaaaaa gcttcttttt cttctggtgt aagcagctgt tgttcattca gccaatctac |
| | | 2221 | tgccaattgc ggtactctcg acaaatagct taatatctcg cggaaacatc gtgtcttgtc |
| | | 2281 | ttccgccccc acagaatagc tgtcccggag cgcataataa gccagcagcc ggcgagtgaa |
| | | 2341 | accataatct cgtttctgcc catcgctcaa ttgctctttt ccggtatgtt taaatccttt |
| | | 2401 | caccgctccc agcatccgat agacattact ccgatggcag aaaaatgatg ccagaaaaat |
| | | 2461 | aacccccgcc gccgtaatca tatatgtacc gttatgcaat tcaaataatg gcggcacaac |
| | | 2521 | gcctttatag tcgctttctt ttgtcgctcc gacagtcttt gattccgcgt tcttgtatat |
| | | 2581 | ttcttccaga aaaaccttga cggcatcttg gtctccaaac cgcagacagt cttctgcgtg |
| | | 2641 | gtaataatgc gagaaataat tgcgaatgtc tttaagtcgc ttctgaattc catccactac |
| | | 2701 | ttgtgatttt atttgctcat cagaaatccc tcccttgctg cgcagcacct cactcagaag |
| | | 2761 | agtcctacat cctttatgat cacagttatc atatgcaata ttcaaagcaa ctgcaaaaca |
| | | 2821 | agacttgttg atgcctcgat aacagtcact tgtaagcgaa taatcaatac ccataatggc |
| | | 2881 | tccttaaata gtttaaaagc tgatagaata acttatattc tctcatatat tgtttttatg |
| | | 2941 | tgtttgaaca tcgtatacat attctgccct taaattactt cagcccgtga tggtgataca |
| | | 3001 | caactcgatt cgatgctgcc gtccggcaca aatgtactgt ttcagtacaa ccgatttaaa |
| | | 3061 | aatgcccccg aattatataa aatctccctc tgctatacaa acaatatttc ccacatatca |
| | | 3121 | actccacttc ccccccacaa attccaaatg ctcaacttaa aattcccacc tcttcaaatc |
| | | 3181 | gacaatctgc cccccgcctc cttgaattct tgtgttcttg agttcttgtg ttcttttatc |
| | | 3241 | ttttaatttt ctaaattatt taaaaattat aagcaacttt tcctccccat ccgacatata |
| | | 3301 | tatagggcct gctcaggaag gacaggcaga aaaaaataag ggctaaaaag gagctgcaaa |
| | | 3361 | agggctaaaa ggaggctgca atgaacagaa aaaaatcaaa ccaaaacctg catacgtata |
| | | 3421 | tacctatata cttacatacc attatcctgt taattcccta aatcccgtcc aaaaggaatt |
| | | 3481 | ttatcgaaac atatccattc catacagcat ctgaatggca tagtattgac tgaacacaaa |
| | | 3541 | agcgataaaa atggagacag aactgatgaa atggtttaca gtgggtttgg tttgccttgc |
| | | 3601 | gggaacgatt gcccaggcgg aaaagcagct tgtggattat gttaatccgt ttct |

| | | | |
|---|---|---|---|
| Cas13 | SEQ ID | 1 | aaccgcaaag ctgctcgcag ccatcgacgc cgcacaataa gacgattaca tacgataggc |
| b-t6 | NO: 5259 | 61 | ataaaattga aaaacatacg caaatgtcac tggaattcct tgtagcgttg tagtactagt |
| | | 12 | tttcctcgcc ggttttctgg tattccgcct tggccttttg gtaggcatcc ttaagctctt |
| | | 181 | ctatagccgt attgaatgca ttattggttt tttgcagatt ctcatctcca gcttccttga |
| | | 241 | gttcggcgag cttctgttgc gtagcgttca gtttgctgtc aagattagag cttagttttt |
| | | 301 | gccagttttc ttttcctgct tccttcatat ctgccatcaa ctgctgagtg tcatttttta |
| | | 361 | attgatcata agtagtatta gcttttttca aaatggcctc cttttgttcg tttagatacg |
| | | 421 | ttccggtagc ctcggccgcc tcggagattt cctctttaac ctgattagca tcggccgagc |
| | | 481 | tcgatgttcc ggaatcctgc tttttgcatc ccaaaggcaa aataacgaaa ctcaatatca |
| | | 541 | gtaacaaaat cttcatagtt ttcatgatct actcctttaa aaataatatt cttttctttt |
| | | 601 | acaccttatc atttttataa aaactcacga aaaatgccaa taaggaattt actgatttgg |
| | | 661 | tatcagttat atactgattt taaccgccaa gtaaatcaga cgctggcagt aattagccgg |
| | | 721 | ggccgaagtg ctttttcttt ttgatactgc tacggtcaat ccctaatttt aattaatgaa |
| | | 781 | aattgccaca gagcgcaacg agtattcaga gaaatcgaaa caggtagggt gggcctcggc |
| | | 841 | ccaccgatca aagcttatcg aaaatgtacc ccctgtggag caggcatacg cctttaattt |
| | | 901 | accctgagaa tcttcgttaa agcttaaaga aagggctgaa cctcaaaatc cctccccaaa |
| | | 961 | atccctccca tactttcaaa atttctaatg ttccacggtc ttcaagattt tttgtcttcc |
| | | 1021 | tcaaggccat tggaaaactc cgtcttcgta tctttgaata gatgtttgat accattccat |
| | | 1081 | atttcttcgg taccttcttt aagctcttcc cagccaatgt tatctgattc ctgaatctgt |
| | | 1141 | tttatttttt ctttagctga cttgcacttc ttgcgcaaca gttcaattcg tttttcaaaa |
| | | 1201 | tcctctttta tatctgcttt tgctcgtaga gcatctgttt tcaaaacttt aatttcagac |
| | | 1261 | tcccactcgt ccaactgttg ttgcaatttc tttatataat cttcacgaga ggtcataatg |
| | | 1321 | taggctcctt tcttaagctt tgagctttgc aatcattacg tattgataag gcaaacatta |
| | | 1381 | tcatggggag ggcgaggaga gggggacagt cacctattaa tgtctgtctg tccacgactc |
| | | 1441 | taagctcctc ttttcactct attgttcctg tgactcggac ttgtcctcct tttctttttc |
| | | 1501 | gagctcttct tcgaccttgg cgtcgaattc gtccatatcc ttctccgatc gagccttggt |
| | | 1561 | ctgctcctgt gcctcagttt tgcgctgggc ttccgtatct tcgtttgctt ggagttccgt |
| | | 1621 | ttggggctcc ggcacgggta catccttctt ttccggtatg atgagctctt gcttacattt |
| | | 1681 | ggggcacacc gtttttctgc cggcggaatc ttcaggggct ttgaccccct ttttacagtg |
| | | 1741 | cgggcattcg aaatgaatag ccattttatc tctccttatc tgcctgtgtt tgattgacca |
| | | 1801 | acacttccca tataaatttc actaaaaact tctgcatact tcaaatgtat aatgcttatt |
| | | 1861 | ataatgcggt aattttattc gaagttcaac aataatgccg cggaattaaa tatttttctt |
| | | 1921 | tcttgtctta gtccaagtca cgcagttatg aggttggata gagctttttg atagcgccag |
| | | 1981 | gggcgattta tgcaaaaagt cttcaataca tttgagggag tatagggagt attagggtca |
| | | 2041 | gcccttaatt ttatggacag tcaccctttt tggaattgca aacagccatt aataggttga |
| | | 2101 | ctgtccctag ttttttatat tgtgtttttg taaaaaactt gactatgaag accaaaagaa |
| | | 2161 | acgaaaatgt agaaattaga acaagtgaga aaggactaga aaatgaagaa agctaaaatc |
| | | 2221 | gttctgatct tagttatttt gttgttggct ttggttgtgt ctcttcaaaa tacagaggct |
| | | 2281 | gtcgaaacca aatttctgct aatgacgatc acaatgcctc gggtaattct gcttttattg |
| | | 2341 | acctttacgc tcggatttgt cggaggtatt attacagcca gtttcattct caagaaatca |
| | | 2401 | ggtaagcctc aagagaaaat caaagcagcg cgagagtgaa agtgtatgtc gaattgaaca |
| | | 2461 | gaatgtccaa taactataac taaggacatg gattaaagct gattgttatt atggactgtc |
| | | 2521 | acaatttttg gaactgaaaa cagccattaa taggtgaatg tccctagttt tccaatagca |
| | | 2581 | gcaaacggga aggtagtcaa tcattattta tattttaagg gctggcccct atgactttcc |
| | | 2641 | cagagaaaga atatgtagga tggcgcagct ttgctgccca tgctgatttt tgacacggat |
| | | 2701 | taacgctgtt taacacggtt taggatttgc cgcagagcac atcaaggaca ctgagaaata |
| | | 2761 | ttaaattaca atagtaagtt tatggcttag ccatagaaaa tagaaaattt atgccacttg |
| | | 2821 | tgccacaggt ataggtatcg taaatgtcaa atcggtgtct gaaaccttta attttcccga |
| | | 2881 | aggaagtcaa tcattattta tattttaagg gctggccccc tatgcctcca cagctttcta |
| | | 2941 | cttctcccga taaggttgag caccgctgtt acttccccac aaattgaggc ccatcacagc |
| | | 3001 | ctgtcaatat cgacgtcgat tgcaaaaaga gctgttactt ccccacaaat tgaggcccat |
| | | 3061 | cacagctcac tcttggtaaa ccatactcaa tagactgctg ttacttcccc acaaattgag |
| | | 3121 | gcccatcaca gcggagtaac atggcagaga caggatataa ttgctgttac ttccccacaa |
| | | 3181 | attgaggccc atcacagcaa taataatatc attaaacgat aaattcgcgc tgttacttcc |
| | | 3241 | ccacaaattg aggcccatca cagcataatc cctggagagg agaaagagtt cactgctgtt |
| | | 3301 | acttccccac aaattgaggc ccatcacagc gactatgtgc tgtacgaggc agatagcttc |
| | | 3361 | gctgttactt ccccacaaat tgaggcccat cacagcatca ccttataagt aacaatacac |
| | | 3421 | caataagctg ttacttcccc acaaattgag gcccatcaca gcaatcagcg gcttgagcgg |
| | | 3481 | cgcatcggta cggctgttac ttccccacaa attgaggccc atcacagcca ttgtatttta |
| | | 3541 | acctttttat tgacagatag ttatctttaa gtttgttttt caaaaagtac atatatatgc |
| | | 3601 | ttctcattcc gtcattatta tcattttttg atttcattta tcaatagctt tgctttttca |
| | | 3661 | aaagaagtcc catcaggaat ctcgccgtgc aaggctttat tccttgcatg tttgagttta |
| | | 3721 | gagagttttt ctgtgtcccg tcctttttta ataagctcat catgtatttg tgtaaaactt |
| | | 3781 | atatgagtct cctcgtcatt aaatttgctt ttttcgataa tcctattttc gaaaatatat |
| | | 3841 | tcctcaaaac agagaacttg tttgataaat tccatacgtt gcttttctat gatatccact |
| | | 3901 | ttatccaaaa tagctttttt ggattcgtaa gccgcaaatg cataactatc gatattgtca |
| | | 3961 | acatattttc ggttcattgc atacacaagc ttggggtaat aggaaagcgg gatatcttct |
| | | 4021 | tttaccttat cggatatttt atatgtcatc cttgtttgtt taagctcgcc taattttgtt |
| | | 4081 | ttctccttta attcaaatcc aagccgctcc aaaagataga taaccataaa tgcgataagt |
| | | 4141 | ttgttctttt cacaaaggcc gtttatcagt gtcatcttat cgaaattctt tttatcaaaa |
| | | 4201 | aattgccttg aaagttcttc atattttccc gatagctcaa ttttacaata atcctctctt |
| | | 4261 | attcttttcg atattttctc agacgaattt tgctggatat gattcttcac aaaacctttg |
| | | 4321 | ggcaaagcta tcctgttcat aaccgtctgc ctgcttttat tcgtgtcaat gcttattcta |
| | | 4381 | agattaagat tctcgatgcc gtgcattttc tttagttcag cggtaatcct ttgtttcatg |
| | | 4441 | attgtaagtt tctgattgtc tgatatctgc ttctttatct gcccggaata ttcaacccag |
| | | 4501 | tccttttcct gccatttcac acctaactcc tgtgacagaa gccttaaatt ctccaggtca |
| | | 4561 | ttaacgtaat taatctgctg atatttttca aggtcatttt ctttaaggct tcgtacttga |
| | | 4621 | tttctcatat cttcaagttt ctttttgttc tctttccgtg ctaattgata ggcttcttct |
| | | 4681 | agatttcttt tattccacaa ttccgtcggc aaaaacctcg tccatttttt actgtcaaac |
| | | 4741 | tcgcgcttaa tgttatgctt ttccttattc cagaaacgaa ccatctttaa gtatcgtcta |
| | | 4801 | taatcctttt gcctcaactt ctcatcagac ggaaggcaca tattgataaa ttccatcact |
| | | 4861 | tcaatcattt tgtcataagc tttatccgtc actttagatg taggatccat cctcttatca |
| | | 4921 | agccggttta gaatcttttc ttttagggtc cctgttcctg cccggccaaa gtaatttgca |
| | | 4981 | gggaaatatt ctcttttaag caattcatct tcgtcaactt gctgcttgct gctgacattc |
| | | 5041 | tgaaattttt tgagaaattg tataatttta ccttccaagg aatcaacgtt ttgcttctca |
| | | 5101 | attaaaattg tgtaacaaag ctccttcaat tgatcttcac taatcactcc cttatgtcca |
| | | 5161 | tttaacgtaa aataactatt gccttgaaaa ggctctatcc atgtaaaggt atccttttcg |
| | | 5221 | tgtttgagtt ctcctctttt ctcttttagc ctgttgctct gatatgtata aaattgcatt |
| | | 5281 | ttattgaaga tgccgctctc gtttaaaaaa gtcgaagcta tacgatggaa aaacaaaccc |
| | | 5341 | ttaccgagct cgtctgactg ctgctgcttc tcaatatgat catcctgctc agatagatga |
| | | 5401 | ttgaccagag cgaatagaag gaaatgcttc tgcatatcag gcacaacctt atatccctcc |
| | | 5461 | ctcacactat aataagtgaa tagatttctt ctcggttgtc cttctttatc attcctttta |
| | | 5521 | aaaccactaa cggaacttat aagcttgttt gcctgtgatt tttttaagaa catacacagc |
| | | 5581 | aaaaacagca cgccagaatc cgtgagacag ttgttgtttt ctatgattac aagtttaaca |
| | | 5641 | tttttacttc cggttgcttc tatagcaatt tgataatatt tttccagtat aggtttttca |
| | | 5701 | ccgttactta gtatcttgac ggtatcatta tgaacgtaat gtgagtaaaa attacgtagc |
| | | 5761 | tctactaatt tcaacaatat agcctcaatt tcagttcttg cgtttttagc cacatcttta |
| | | 5821 | aagttgtaga tataattctc cacacttttt aataataagc ggttattgaa aatcttatca |
| | | 5881 | atcactttct ttgcattttc cttgttgctg attatgtgcg gaatttgttt ttcaataatt |
| | | 5941 | tcatcaagcc ctgaaagatt gaggatcgtc tgattaaaat aaaacgcagc ttcttctttt |
| | | 6001 | tttaatttaa tgatgttcat aattaaatac ctccataaca acaaagattt tgtcaggcga |
| | | 6061 | aaaaaccaat tttaaaaacg ctcttacaaa tacgaatatc ccttatgatt tctcagagca |
| | | 6121 | ggctcttaag tggagttgaa tgcgcaaacc cctagtaaga ctgattaggc gcggtaagac |
| | | 6181 | aaaataaggc gattcgagga agtttttcgt tcagtgattc ttttcacctc aacaaatata |
| | | 6241 | atacacgaaa gaaagtaaaa aggcaaaagt aaaaaagaaa aaactttttt tacaggataa |
| | | 6301 | tgtcggggat gctttactta tagataaaac taagtgatag tcacctataa aatttaagta |
| | | 6361 | acacggtctt attaaaatac tcattctcga aagcttgtat tcgacgaaat aaataataaa |
| | | 6421 | acagaataca gaacacagga gacagaacct ttctattaac gattttcgat tgactattga |
| | | 6481 | ctattttttt tgttaatccg cgtttatctg cgttcatctg cggtttccca tcttctatcc |
| | | 6541 | tatccgtggc cgttttatgg cctattcact atatacagcg tatctcacgg ggtaaatgtt |
| | | 6601 | acctataaaa ttggaaagtt tcgattttta agcaaaatag tgaaaaaaca cgaagaacag |
| | | 6661 | gcctgaaaca agtaaaaagt taaaaggaaa aagggaaaat ttagtcgatt cttgattctg |
| | | 6721 | gaagcggtat ttagcaggga cagtccagtt cttttatcag cttaaatttg agcataatcg |
| | | 6781 | ggcgttaacc aaacggttta gactgacatt ctcttcagca gcctcaattg tgagttcacg |
| | | 6841 | atgcacctca ggaggaacac gaactacgaa tttaccactg tactgctgga agcttagtgg |
| | | 6901 | ttttggtatc ggttctccgt ttttcttcat atctttgata acatcatcca cgacacgaat |
| | | 6961 | aattcccccc acggctcttt cgggagtaga agccaagtaa cttagacttg ggaattctgc |
| | | 7021 | acacagaccg acaaattcgg catcatcttc ggaccaaatt accctgtatg tataataatg |
| | | 7081 | cctaatcttt ggttttattt tcttcataat taaatagtac taatattgat actataagtc |
| | | 7141 | aatgaaaaag ttctaaaaat tttaaaatag ttcaataatc gcgaaaaaca ggcctgaaac |
| | | 7201 | aagtaaaaag ttaaaaggaa aaaggcaaaa aagcgagata aaggcagaat tcaggagaca |
| | | 7261 | ggagacagaa tacaggggaa aaattaaagg gaaaaatgcc ttgtaattga ttatttatta |
| | | 7321 | ttgtatattg actatttctc tgtactttcg gcgccctctg tggccctttt aattttctat |
| | | 7381 | tttctattta ctattgatta tttttgcgca aaaaaaacgc tccatggaat ccacggagcg |
| | | 7441 | tttttatatc aagaacagtt aagccgacag cgaaaatgcc tgacatacac cggctttaga |
| | | 7501 | ttcatcgctt tcgctcagaa tgacatatgg ccgggttcta gtatatccct agaaaggagg |
| | | 7561 | tgatccagcc gcaggttccc ctacggctac cttgttacga cttagtgcca gtcatcgggc |
| | | 7621 | ttaccgt |

Amino acid sequences of Cas13b-t1, Cas13b-t2, Cas13b-t3, Cas13b-t4, Cas13b-t5, and Cas13b-t6 proteins tested in this example are shown in Table 14 below.

TABLE 14

| Protein | |
|---|---|
| names | Sequences |
| Cas13b-t1 | MEFENIKKTSNKEVYSIEQYEGEKKWCFAIVLNRAQTNLEENPKLFEQTLTRFEKIMKQDWFNEETKKLIYEKEEENK |
| SEQ ID | VKEEIQIAASERLKNLRNYFSHYLHAPDCLIFNRNDTIRIIMEKAYEKSRFEAKKKQQEDISIEFPELFEEEDKITSAGVVF |
| NO: 5260 | FVSFFIERRFLNRLMGYVQGFRKTEGEYNITRQVFSKYCLKDSYSVQAQDHDAVMFRDILGYLSRVPTEIYQHIKLTRK |
| | RSQDQLSERKTDKFILFALKYLEDYGLKDLADYTACFARSKIKRENEDTKETDGNKHKFHREKPVVEIHFDKEKQDQF |
| | YIKRNNVILKAQKKGGQSNVFRMGVYELKYLVLLSLLGKAEEAIQRIDRYISSLKKQLPYLDKISNEEIQKSINFLPRFV |
| | RSRLGLLQVDDEKRLKTRLEYVKAKWTDKKEGSRKLELHRKGRDILRYINERCDRPLSRKEYNNILKFIVNKDFAGFY |
| | NELEELKRTRRLDKNIIQKLSGHTTLNALHERVCDLVLQELGSLQSENLKEYIGLIPKEEKEVTFREKVDRILEQPVVYK |
| | GFLRYEFFKEDKKSFARLVEEAIKTKWSDFDIPLGEEYYNIPSLDRFDRTNKKLYETLAMDRLCLMMARQYYLRLNEK |
| | LAEKAQHIYWKKEDGREVIIFKFQNPKEQKKSFSIRFSILDYTKMYVMDDPEFLSRLWEYFIPKEAKEIDYHKHYARAF |
| | DKYTNLQKEGIDAILKLEGRIIERRKIKPAKNYIEFQEIMNRSGYNNDQQVALKRVRNALLHYNLNFEREHLKRFYGVV |
| | KREGIEKKWSLIV |
| Cas13b-t2 | MQVENIKKGSSQGMYSIEQYEGAKKWCFAIVLNRAQTNLQGNPKLFEETLTRFERIRKEDWFDQETKKLIYAKQEQNE |
| SEQ ID | VEEEIQKAADEKLRDLRNYFSHYFHTPDCLIFTQNDPVRIIMEKAYEKARFEQAKKEQEDISIEFGELFEENGRITSAGV |
| NO: 5261 | VFFASFFAERRFLNRLMGYVQGFTRTEGEYKITRDVFSTYCLRDSYSVKTPDHDAVMFRDILGYLSRVPSESYQRIKES |
| | QMRSETQLSERKTDKFILFALNYLEDYGLEDLADYTACFARTRIKREQDENTDGKEQKPHRKKPRVEIHFERAEGDPF |
| | YIKHNNVILRTQKKGAQTYIFRMGVYELKYLVLLSLLGKGAEAVKRIDRYVHSLRNQLPHIEKKSTEEIEGYVRFLPRF |
| | VRSHLGLLGVDDEKKIKARVDYVKAKWLEKKEKSRELQLHRKGRDILRYINERCERPLNIDEYNRILELLVTKHLDGF |
| | YRELEELKKTRRIDKNIVCNLSRHKSVNALHEKVCDLVVQELESLGREELKEYVGLIPKEEKEVSFEEKTDRVVKQPVI |
| | YKGFLRNEFFRESRKSFARLVEEAVREKGEVYDVPLGGEYYEIVSLDTFDKDNKRLYETLAMDRLLLMIARQYHLSLN |
| | KELAKRAQQIEWKKEDGEEVIIFTLKNPAQPEQSCSVRFSLRDYTKLYVMDDAEFLARLCDYFLPKDEEQIDYHRLYT |
| | QGMNRYTNLQREGIEAILELEKKTIGPEQPRPPKNYIPFSEIMDKSAYNEDDQKALRRVRNALLHHNLNFARADFKRFC |
| | GIMKREGIEKRWSLAV |
| Cas13b-t3 | MAQVSKQTSKKRELSIDEYQGARKWCFTIAFNKALVNRDKNDGLFVESLLRHEKYSKHDWYDEDTRALIKCSTQAA |
| SEQ ID | NAKAEALRNYFSHYRHSPGCLTFTAEDELRTIMERAYERAIFECRRRETEVIIEFPSLFEGDRITTAGVVFFVSFFVERRV |
| NO: 5262 | LDRLYGAVSGLKKNEGQYKLTRKALSMYCLKDSRFTKAWDKRVLLFRDILAQLGRIPAEAYEYYHGEQGDKKRAND |
| | NEGTNPKRHKDKFIEFALHYLEAQHSEICFGRRHIVREEAGAGDEHKKHRTKGKVVVDFSKKDEDQSYYISKNNVIVR |
| | IDKNAGPRSYRMGLNELKYLVLLSLQGKGDDAIAKLYRYRQHVENILDVVKVTDKDNHVFLPRFVLEQHGIGRKAFK |
| | QRIDGRVKHVRGVWEKKKAATNEMTLHEKARDILQYVNENCTRSFNPGEYNRLLVCLVGKDVENFQAGLKRLQLAE |
| | RIDGRVYSIFAQTSTINEMHQVVCDQILNRLCRIGDQKLYDYVGLGKKDEIDYKQKVAWFKEHISIRRGFLRKKFWYD |
| | SKKGFAKLVEEHLESGGGQRDVGLDKKYYHIDAIGRFEGANPALYETLARDRLCLMMAQYFLGSVRKELGNKIVWS |
| | NDSIELPVEGSVGNEKSIVFSVSDYGKLYVLDDAEFLGRICEYFMPHEKGKIRYHTVYEKGFRAYNDLQKKCVEAVLA |
| | FEEKVVKAKKMSEKEGAHYIDFREILAQTMCKEAEKTAVNKVRRAFFHHHLKFVIDEFGLFSDVMKKYGIEKEWKFP |
| | VK |
| Cas13b-t4 | MPVNYSLDQDYYKGTHKSCFTVPLNIAWDNGSKKGCENLLKEAMRTRGGFTQEDIEKVHRSLAEKLNGIRDYFSHYY |
| SEQ ID | HEDKPLEFKKGDDDAVKDFLEKTFSYAAGETQKRVKESGYQGIIPPIFELCGDQVRITAAGVIFLASFFVPRSTLERMFG |
| NO: 5263 | AVQGFKRSDRGDLDTGQKRDYYFTRSLLSFYTLRDSYYLQADETRPFREILSYLSCVPFDSVQWLQAHGKLSKSEEKE |
| | FFGRPVEEQDEENPAQTEKQTAPAGRRMRKKNKFILFAVRFIEAWARNEKLSVEFGRYRNIQNEEDRRKQSGKKVREV |
| | FFPSALNNLSAEEQDLEGLLYIRNNHALIRIHLKAKTPVTVRISEHELMYLVLAILSGKGGNAVQKLSKYVWDVRMRS |
| | RGPLTNMPRNFPAFLRSPASEVSEQAVQNRLNYIRKTLKEIQANLQKEAQTGQWILDKGQKIRHILRFISDSMPDFRRRP |
| | SVKEYNELRELLQTLAFDDFYRKLASFQTERKLDAAVWNNLAQCKSINELCERCCQLQQQRLDELEKQGGDELKRYI |
| | GLLPKEKGKHYEEQNTPARKFERFIENQLSVPKYFLRCKLFVTGGSRRTNLLKLVQEHLKPKTSVFHEERLYLREEQPG |
| | DYPWSDRKIIQKMYYLYVQDLLCMQMAQWHYEHLTPQVKGKIDWEINSESKESDGYNRFKVEYKGPQGCRIIFRVQ |
| | DFGRLDFLNKAPMLDNICQWFLSGRKEITWPEFLRDGLQRYRQRQILVVRALFRFEENLKIPEEEWKGKSHLSFDEVLE |
| | RFSGKNRLSEEEKESIRRVRNDFFHEEFEATPSQWRDFERRMSEYLNKEKREKPKKKKR |
| Cas13b-t5 | MGIDYSLTSDCYRGINKSCFAVALNIAYDNCDHKGCRTLLSEVLRSKGGISDEQIKSQVVDGIQKRLKDIRNYFSHYYH |
| SEQ ID | AEDCLRFGDQDAVKVFLEEIYKNAESKTVGATKESDYKGVVPPLFELHNGTYMITAAGVIFLASFFCHRSNVYRMLGA |
| NO: 5264 | VKGFKHTGKEQLSDGQKRDYGFTRRLLAYYALRDSYSVGAEDKTRCFREILSYLSRVPQLAVDWLNEQQLLTPEEKE |
| | AFLNQPAEDEGGDISDSSSSDKNKKSKEKRRSLRRDEKFILFAIQFIEGWAAEQGLDVTFARYQKTVEKAENKNQDGK |
| | QARAVQLKYRNQGLNPDFNNEWMYYIQNEHAIIQIKLNNKKAVAARISENELKYLVLLIFEEKGNDAVQKLNCYIYS |
| | MSQKIEGEWKHRPEDERWMPSFTKRADRTVTPEAVQSRLSYIRKQLQETIEKIGQEEPRNNKWLIYKGKKISMILKFIS |
| | DSIRDIQRRPNVKQYHILRDALQRLDFDGFYKELQNYVNDGRIAVSLYDQIKGVNDISGLCKKVCELTLERLAGLEAK |
| | NGSELRRYIGLEAQEKHPKYGEWNTLQEKAKRFLESQFSIGKNFLRKMFYGDCCQKRCFDEEKGYNTQAKERKSLYSI |
| | VKEKLKDIKPIHDDRWYLIDRNPKNYDNKHSRIIRQMCNTYIQDVLCMKMAMWHYEKLISATEFRNKLEWNCIGQGN |
| | MGYERYSLWYKTGCGVVIQFTPADFLRLDIIEKPAMIENICQCFVLGNKKLNSGAEKKITWDKFNKDGIAKYRKRQAE |
| | AVRAIFAFEEGLKIQEDKWSHERYFPFCNILDEAVKQGKIKDTGKDKEALNRGRNDFFHEEFKSTEDQQAIFQKYFPIV |
| | ERKDDTKKRRDKKQK |
| Cas13b-t6 | MNIIKLKKEEAAFYFNQTILNLSGLDEIIEKQIPHIISNKENAKKVIDKIFNNRLLLKSVENYIYNFKDVAKNARTEIEAIL |
| SEQ ID | LKLVELRNFYSHYVHNDTVKILSNGEKPILEKYYQIAIEATGSKNVKLVIIENNNCLTDSGVLFLLCMFLKKSQANKLIS |
| NO: 5265 | SVSGFKRNDKEGQPRRNLFTYYSVREGYKVVPDMQKHFLLFALVNHLSEQDDHIEKQQQSDELGKGLFFHRIASTFLN |
| | ESGIFNKMQFYTYQSNRLKEKRGELKHEKDTFTWIEPFQGNSYFTLNGHKGVISEDQLKELCYTILIEKQNVDSLEGKII |
| | QFLKKFQNVSSKQQVDEDELLKREYFPANYFGRAGTGTLKEKILNRLDKRMDPTSKVTDKAYDKMIEVMEFINMCLP |
| | SDEKLRQKDYRRYLKMVRFWNKEKHNIKREFDSKKWTRFLPTELWNKRNLEEAYQLARKENKKKLEDMRNQVRSL |
| | KENDLEKYQQINYVNDLENLRLLSQELGVKWQEKDWVEYSGQIKKQISDNQKLTIMKQRITAELKKMHGIENLNLRIS |
| | IDTNKSRQTVMNRIALPKGFVKNHIQQNSSEKISKRIREDYCKIELSGKYEELSRQFFDKKNFDKMTLINGLCEKNKLIA |
| | FMVIYLLERLGFELKEKTKLGELKQTRMTYKISDKVKEDIPLSYYPKLVYAMNRKYVDNIDSYAFAAYESKKAILDKV |
| | DIIEKQRMEFIKQVLCFEEYIFENRIIEKSKFNDEETHISFTQIHDELIKKGRDTEKLSKLKHARNKALHGEIPDGTSFEKA |
| | KLLINEIKK |

### Example 3—Identification and) Characterization of Exemplary Small Cas13 Proteins

CRISPR-Cas13 systems can be used for precise RNA editing, an attractive therapeutic strategy when temporary changes are desirable or DNA editing is not possible. In this example, Applicants identified and characterized an ultra-small family of Cas13b, Cas13b-t, and showed it mediates mammalian transcript knockdown. By functionalizing Cas113b-t with adenosine and cytosine deaminase domains, Applicants engineered compact variants of REPAIR and RESCUE RNA editors, which may be more amenable for in vivo use.

RNA-targeting CRISPR-Cas13 systems can be harnessed for a variety of applications (1), including precision base editing (2, 3). RNA base editing is a therapeutic strategy that allows for installation of temporary, non-heritable edits. Cas-13-based RNA editing systems with smaller sizes are needed because they are better compatible with the packaging capacity of delivery systems, such as adeno-associated virus, a widely used viral vector for gene delivery (4, 5).

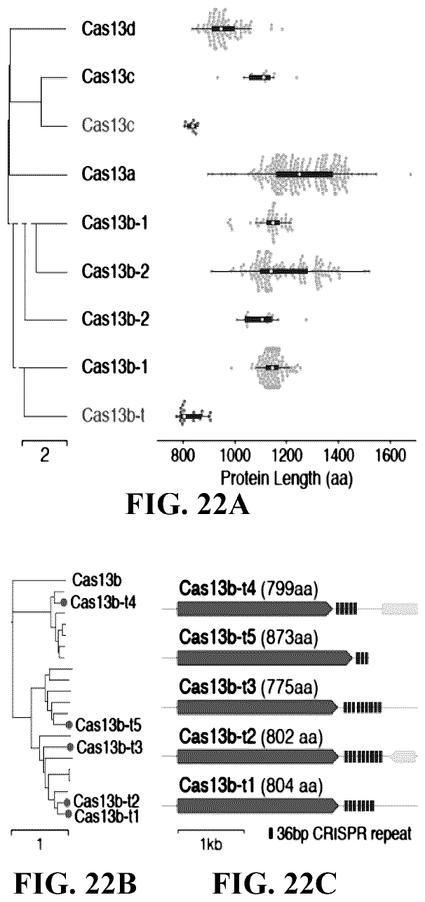

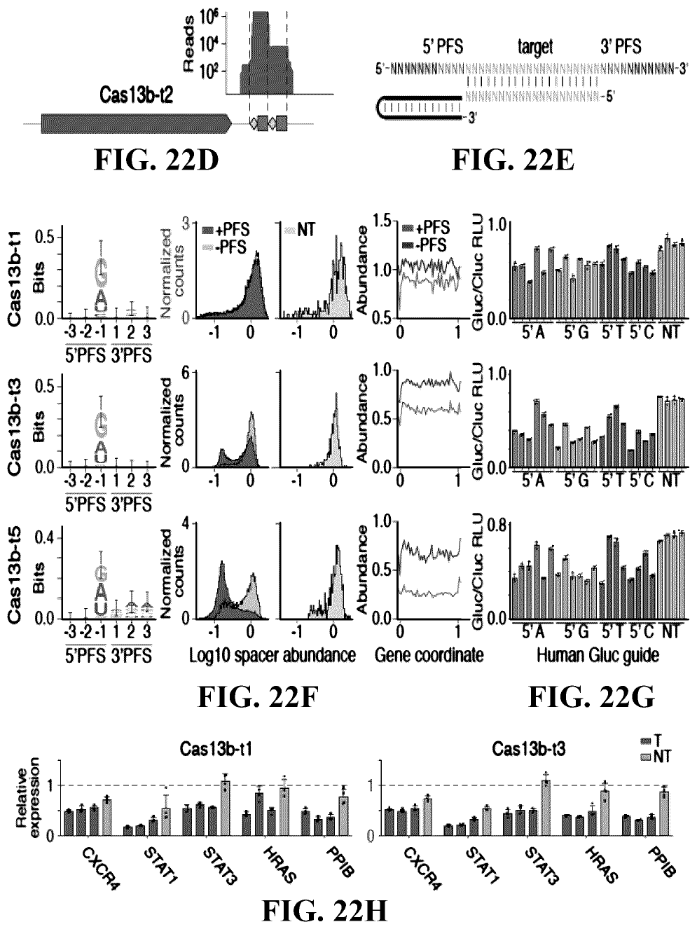

To overcome this limitation, Applicants performed a computational search of prokaryotic and viral genomes and metagenomes for small Cas13 orthologs, identifying 4726 candidates. Phylogenetic analysis revealed two novel groups of ultra-small Cas13 proteins that form distinct branches within the Cas13b and c subtypes. (FIG. 22A). Unlike other Type VI-B CRISPR-Cas loci (6), the genomic loci encoding Cas13b-t lack any accessory genes. In this example, Applicants focused on the new tiny Cas13b (Cas13b-t) subfamily (FIG. 22B).

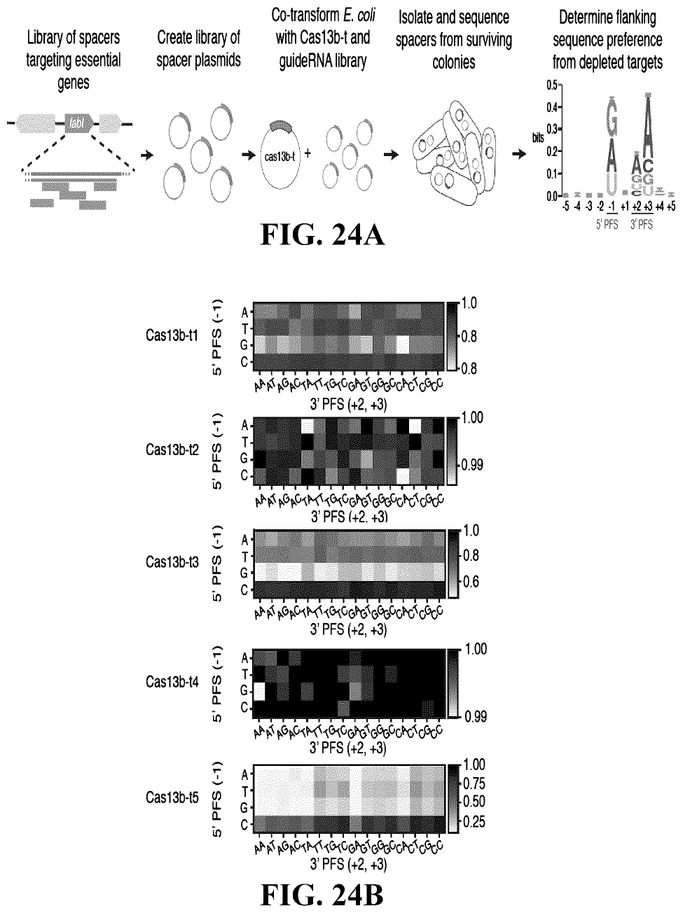

To experimentally characterize Cas13b-t, Applicants first identified the CRISPR RNA (crRNA) components. Applicants transformed E. coli with a plasmid containing the Cas13b-t2 locus (FIGS. 22B-229C) with the CRISPR array truncated to two direct repeats (DRs) and performed small RNA sequencing. Applicants found that the crRNA of Cas13b-t2 has a 3′ DR (FIG. 22D). To determine if Cas13b-t is capable of mediating nucleic acid interference, Applicants performed a negative selection screen using a library of crRNAs that consist of a spacer followed by the DR and target essential gene transcripts in E. coli6 (FIG. 24A). Three of the five tested members of the Cas13b-t subfamily, Cas13b-t1, 3, and 5, mediate depletion of targeting spacers in E. coli (FIG. 22F). Mapping of depleted spacers to the E. coli transcriptome and analysis of the flanking sequences revealed that all three active orthologs have a permissive 5′ D (A/G/T) protospacer flanking sequence (PFS) preference (FIGS. 22F and 24B). Additionally, assessment of the normalized position of depleted spacers along the target transcript indicates no positional preference within the coding region and enhanced depletion when targeting the 5′ UTR (FIG. 22F).

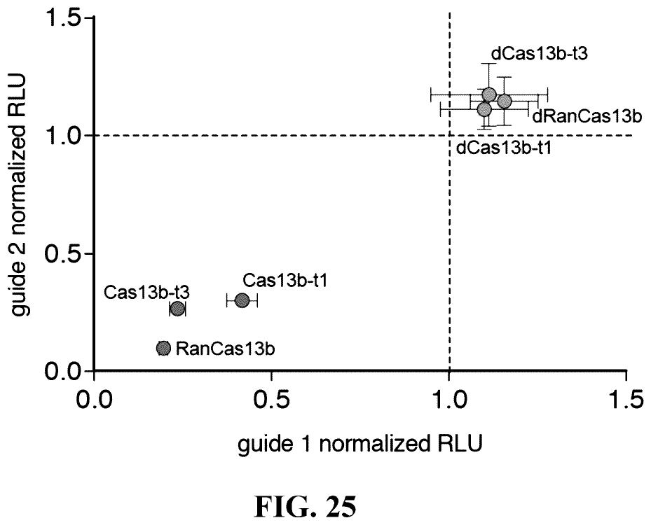

To evaluate Cas13b-t-mediated knockdown and the importance of the PFS for RNA targeting in human cells, Applicants tested the three active Cas13b-t's using a set of 20 guideRNAs (gRNAs) with spacer sequences targeting regions with different adjacent 5′ bases in a Gaussia luciferase reporter. Applicants found that all three proteins promoted knockdown in HEK293FT cells with varying efficiencies, from 50% to 75% for the most efficient gRNA tested (FIG. 22G). Mutation of the HEPN domains in Cas13b-t1 and 3 (dCas13b-t1 and 3) abolished the knockdown activity (FIG. 25). Further, Applicants found that the PFS preference detected in E. coli was not manifested in HEK293FT cells, indicating that the PFS has little effect in mammalian cells, similar to previously studied Cas13′s2 (FIG. 22G). Applicants next targeted endogenous transcripts in mammalian cells with Cas13b-t1 and 3, the smallest and most active members of the tested Cas13b-t's. Both proteins mediated knockdown of five target transcripts for all gRNAs tested (12-68% and 27-64% knockdown compared to a non-targeting gRNA for Cas13b-t1 and 3, respectively) (FIG. 22H).

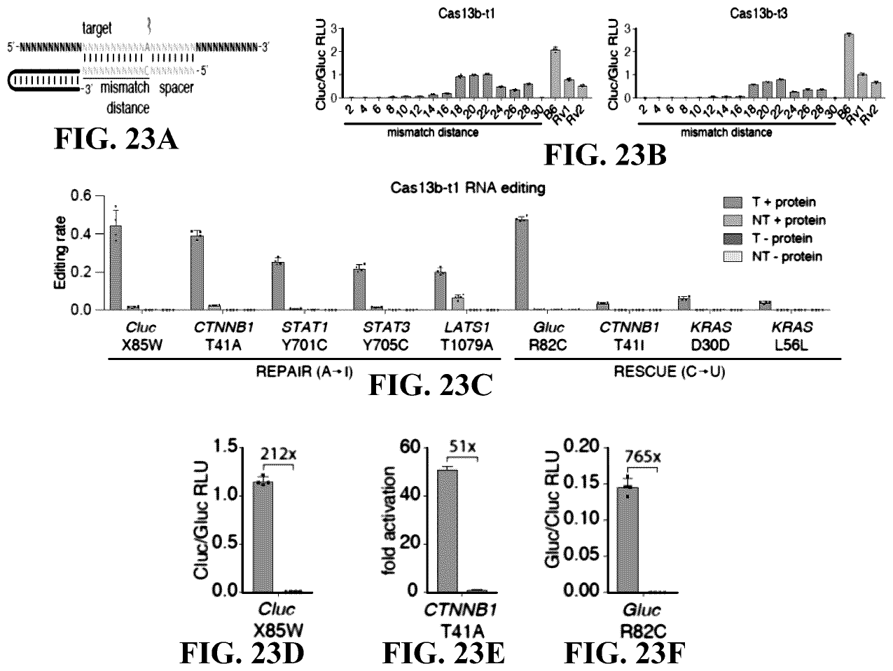

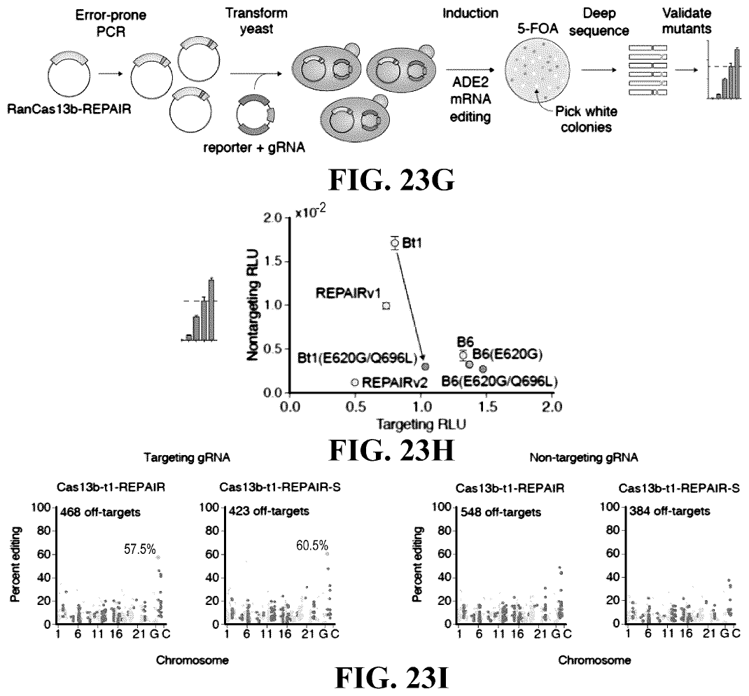

To test the capacity of Cas13b-t's for RNA editing, Applicants fused dCas13b-t1 and 3 with a hyperactive mutant of the human adenosine deaminase acting on RNA 2 (ADAR2dd(E488Q)) to create Cas13b-t1-REPAIR and Cas13b-t3 REPAIR. Applicants evaluated the ability of these fusion proteins to direct A-to-I RNA editing in HEK293FT cells by attempting to revert tryptophan (W) 85 to STOP (X) mutation in a Cypridina luciferase reporter. Site-specific RNA editing was achieved by introducing a cytidine mismatch in the gRNA spacer sequence across from the target adenosine (2, 7) (FIG. 23A). Spacer sequences were designed to vary the distance between this mismatch and the DR, as variability in the optional mismatch position has been observed for different Cas13b-ADAR fusion proteins and target sites (2, 3). Applicants found that both Cas13b-t1-REPAIR and Cas13b-t3-REPAIR showed optimal editing with a mismatch distance of 18-22 base pairs (bp) in a 30-bp spacer sequence. Editing efficiency was comparable to the previously described REPAIRv1 and v2 systems2 and approximately 50% and 13% of that of the more efficient RanCas13b-REPAIR3 for Cas13b-t1-REPAIR and Cas13b-t3-REPAIR, respectively (FIG. 23B).

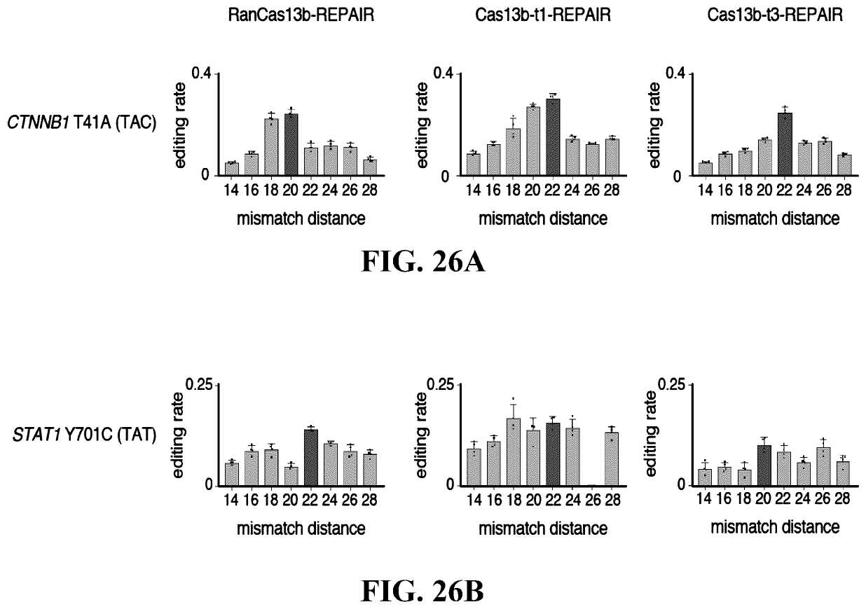

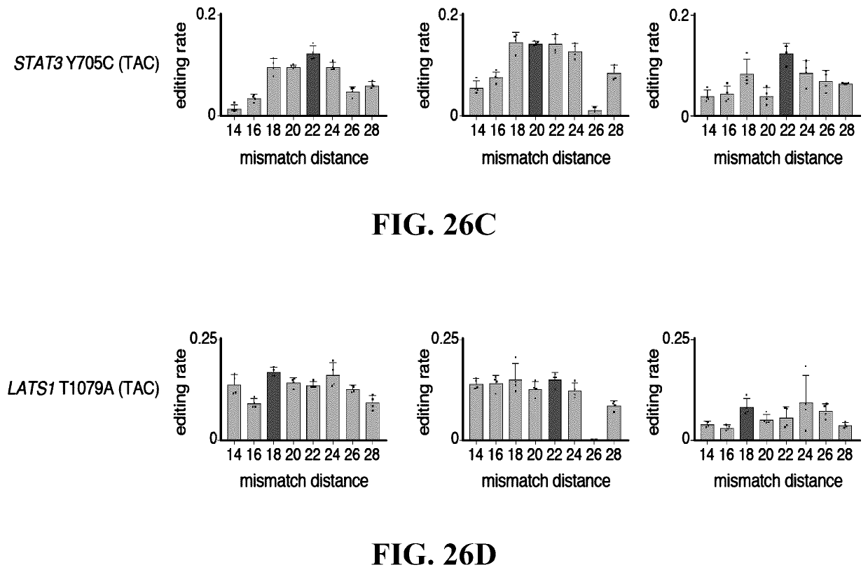

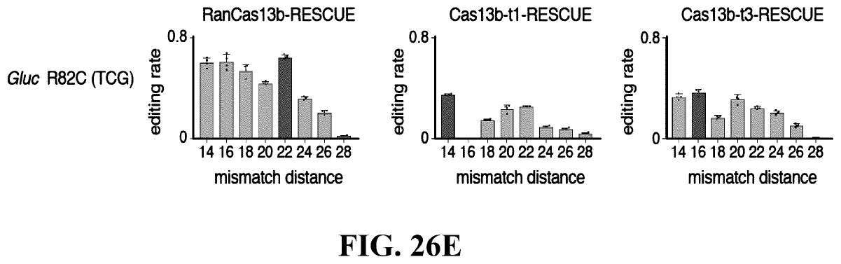

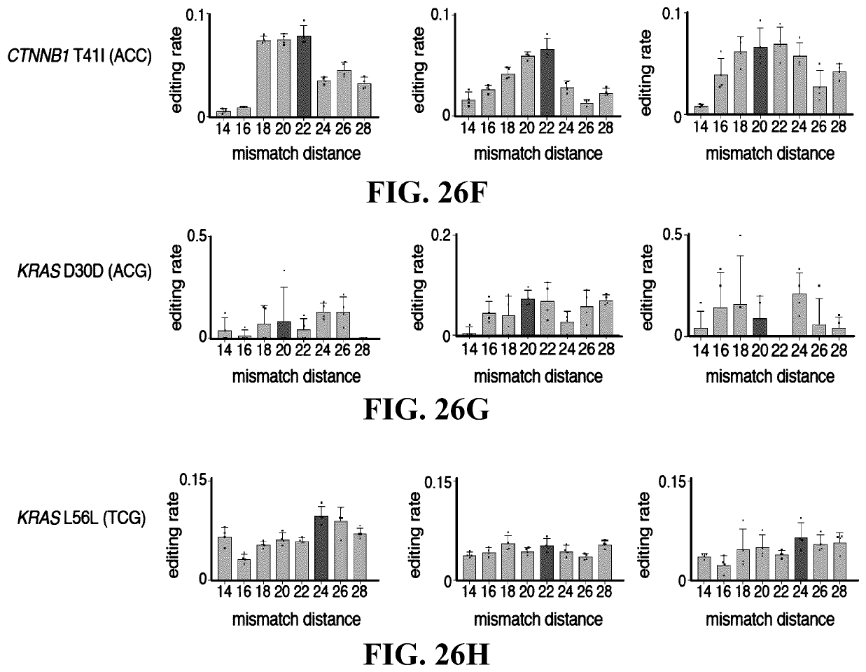

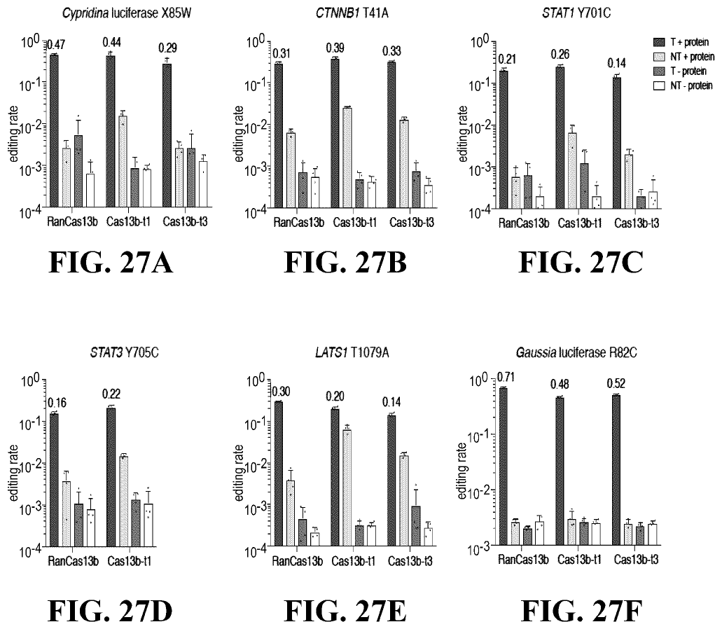

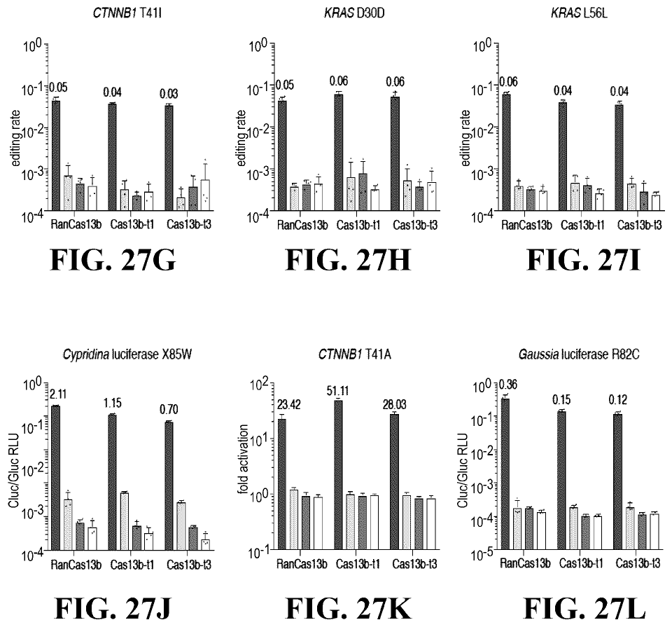

Applicants additionally fused both dCas13b-t1 and dCas13b-t3 with a previously described evolved ADAR2dd capable of cytidine to uridine deamination3 (Cas13b-t1-RESCUE and Cas13b-t3-RESCUE) and directed both editors to reporter and endogenous transcripts in HEK293FT cells (FIGS. 26A-26H). Applicants found that these fusion proteins were capable of mediating both A-to-I or C-to-U editing of all targets tested at levels comparable to or better than RanCas13b-REPAIR/RESCUE (FIGS. 23C-23F and FIGS. 27A-27L).

To demonstrate the ability of Cas13b-t-REPAIR to edit functionally relevant targets, Applicants targeted previously characterized phosphorylation sites. In particular, Applicants attempted to alter activation of the Wnt/beta-catenin pathway by editing the threonine (T) 41 codon of CTNNB1, a site known to promote degradation of beta-catenin when phosphorylated (8). Applicants found that Cas13b-t1-REPAIR was able to mediate 40% editing at this site, converting the codon to alanine (A) and leading to a 51-fold increase in beta catenin activity, which may be relevant for promoting regeneration after acute liver failure (9, 10) (FIG. 20E). Cas13b-t1-REPAIR was also able to efficiently edit sites corresponding to phosphorylated residues in the STAT1, STAT3 and LATS1 transcripts (FIG. 23C).

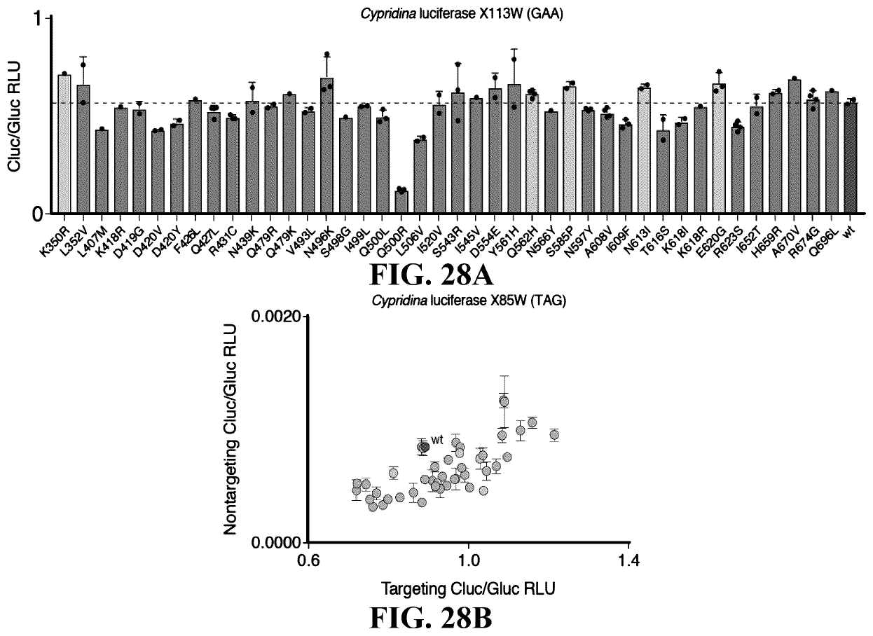

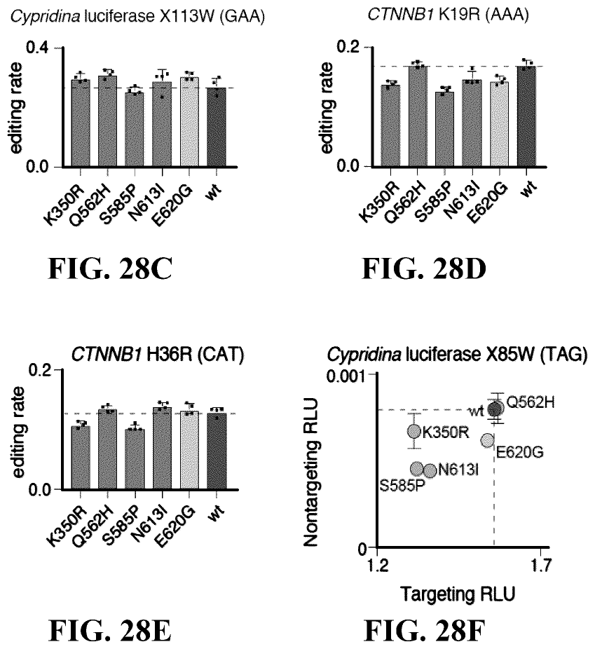

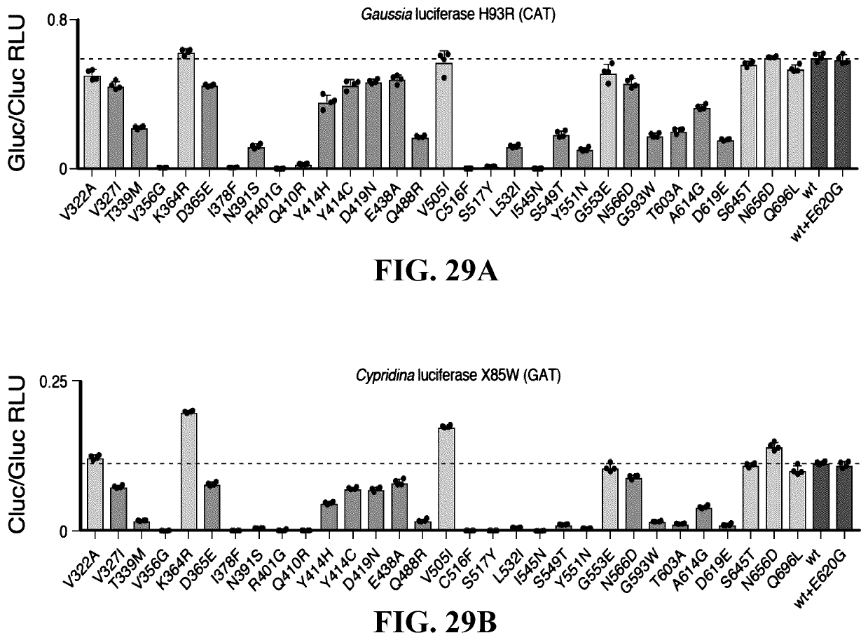

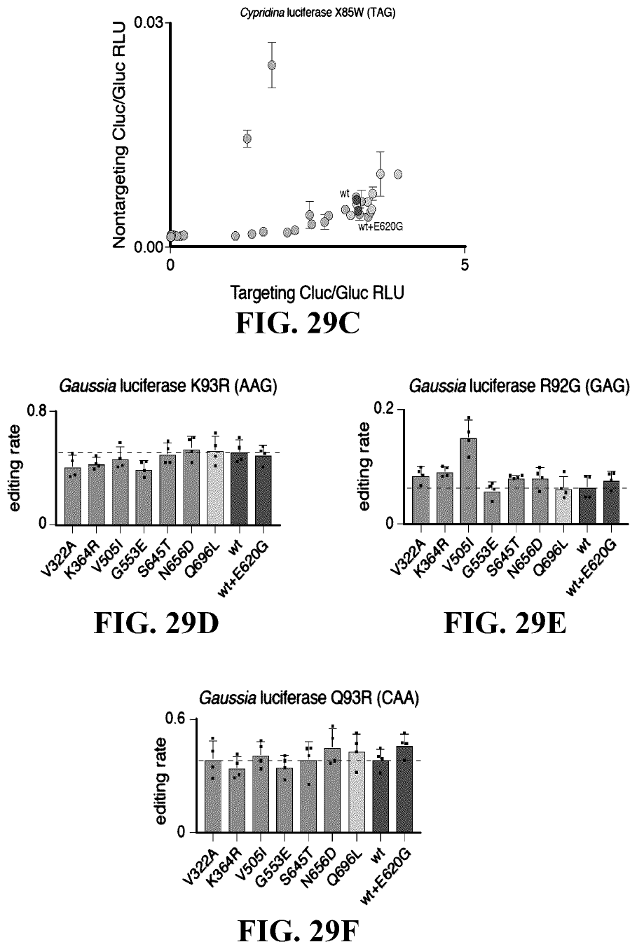

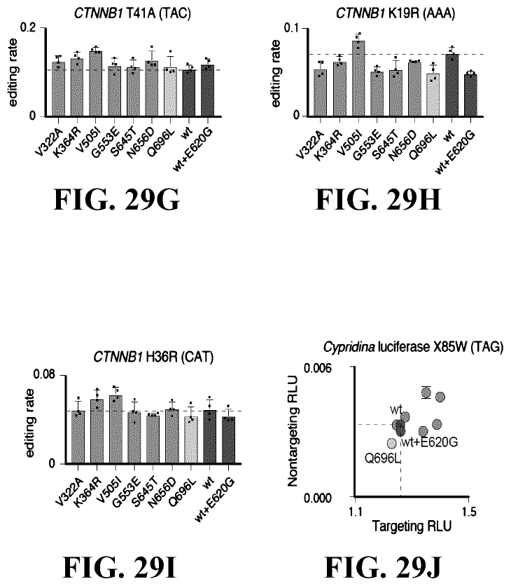

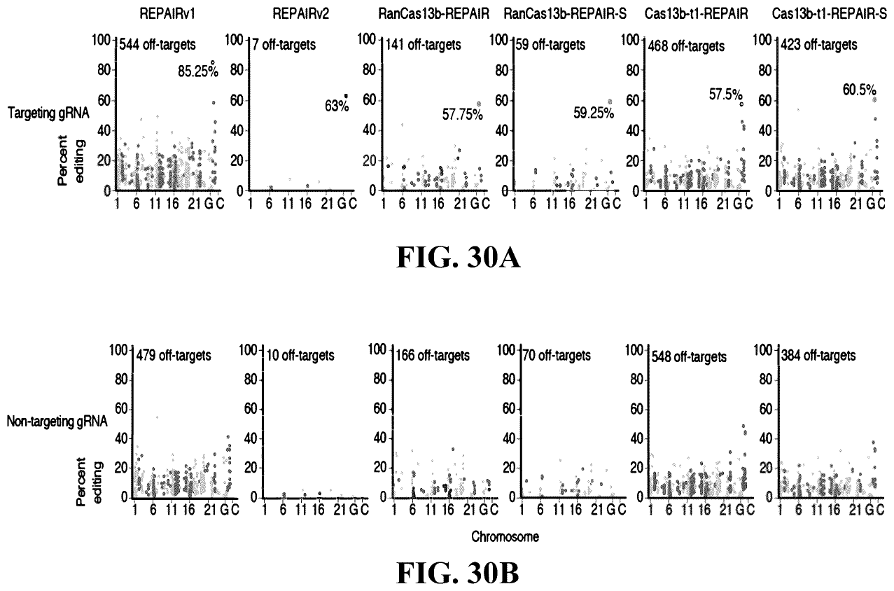

Finally, Applicants evaluated the transcriptome-wide specificity of Cas13b-t1-REPAIR and found the number of off-target edits caused by this system was comparable to REPAIRv1 (FIGS. 30A-30B), which may be due to promiscuous activity of the ADAR deaminase domain (2, 3). To additionally accelerate the translation of REPAIR to therapeutic use, Applicants sought to improve the specificity of Cas13b-t1-REPAIR (FIG. 23G). Through a parallel effort to directly evolve ADAR mutants that are both highly specific and efficient in the context of fusion with dRanCas13b, Applicants identified two promising mutations in ADAR2dd (E620G and Q696L) (FIGS. 28A-28F, 29A-29J). Applicants incorporated these two mutations in Cas13b-t1-REPAIR and found that the number of off-target edits decreased while maintaining comparable on-target activity as the original Cas13b-t1-REPAIR (FIGS. 23H-23I).

The small size and high efficacy of Cas13b-t-REPAIR and RESCUE constructs made them compatible with viral delivery, resolving a major challenge to deployment of this novel therapeutic strategy.

### Methods

### Data Curation and Search Pipeline

Assembled prokaryotic and phage genomic DNA contigs from metagenomes and genomes were downloaded from NCBI, WGS, and JGI, totaling 3.16 trillion bp. All open reading frames larger than 80 aa were annotated resulting in 10 billion putative proteins for further analysis. Previously developed Cas13 profiles (11) were used to identify Cas13 family proteins with HMMER3.212 using a minimum bitscore threshold of 25. A group of small (˜800aa) but divergent Cas13b's were identified and used to seed a second HMMER search with the same settings to retrieve additional members of this subfamily. In total, 4726 Cas13 proteins were identified.

### Phylogenetic Analysis

For phylogenetic analysis and classification, the 4726 candidate genes were clustered using MMseqs2 with a minimum sequence identity of 50% and minimum coverage of 70% (13, 14). Proteins within each cluster were clustered at 90% identity and 80% minimum coverage for redundancy reduction. Each redundancy reduced cluster was aligned using MAFFT15 with default parameters. Proteins identified as truncated or partial and/or clusters entirely composed of them were removed from the analysis.

The aligned redundancy reduced clusters were converted into HHsuite profiles using all columns with less than 50% gaps, and each of these profiles was searched against each other with profile-profile alignment using HHsearch. The resulting pairwise bitscores between clusters, sij, where i,j denote clusters i and j, respectively, were used to construct a classification dendrogram. First, the asymmetric bitscores were symmetrized by setting sᵢⱼ=(sᵢⱼ+sⱼᵢ)/2. Then, pseudo-distances were calculated by setting dᵢⱼ=−(log sᵢⱼ−log min(sᵢᵢ, sⱼⱼ))/2 to generate a distance matrix (16). A UGPMA dendrogram was constructed using these distances. Branches and the subtrees of the dendrogram were contracted without modifying their topology, to highlight known subtypes and subgroups within each subtype. Lengths in amino acids (aa) of the redundancy reduced proteins from each subtree were used to generate protein size distributions.

### Design and Cloning of Bacterial Expression Plasmid Constructs

All cloning in this study was performed using chemically competent Stbl3 E. coli (NEB) unless otherwise noted. All PCR for cloning was performed using 2X Phusion Flash High-Fidelity Master Mix (Thermo Fisher) unless otherwise noted.

The Cas13b-t2 full locus was synthesized and cloned into the BamHI site of pACYC184 by GenScript.

To clone bacterial expression plasmids for the PFS screen, Cas13b-t protein coding sequences were human codon optimized using GeneArt GeneOptimizer (Thermo Fisher) and synthesized by GenScript into a pcDNA3.1(+) backbone. Genes were amplified by PCR to add a pLac promoter and cloned into a pBR322 backbone (NEB) digested with EcoRV (Thermo Fisher) by Gibson assembly.

crRNA expression cassettes for each DR corresponding to each Cas13b-t of interest were synthesized by IDT, amplified by PCR, and cloned into a pACYC184 backbone digested with EcoRV and BamHI (Thermo Fisher) by Gibson assembly. All primers are listed in Table 18 and final constructs in Table 31.

### Design and Cloning of Mammalian Expression Plasmid Constructs

Mammalian gRNA expression cassettes were amplified from pC0048 (Addgene plasmid #103854) (2) using primers to add the DR for each Cas13b-t ortholog of interest and cloned into pC0048 digested with LguI and KpnI (Thermo Fisher) using Gibson assembly.

Mammalian protein expression cassettes were cloned by amplifying previously mentioned synthesized Cas13b-t genes by PCR and cloning into pC0053 (Addgene plasmid #103869) (2) digested with HindIII and NotI (Thermo Fisher), either alone or with addition of a piece including ADAR2dd(E488Q) amplified from pC0053 for REPAIR constructs and pC0078 (Addgene plasmid #130661) (3) for RESCUE constructs. Site directed mutagenesis was used to create catalytically inactivated Cas13b-t's. All primers are listed in final constructs in Table 21.

ADAR2 mutants derived from directed evolution screens were cloned by introduction of mutations via PCR primers listed in Table 21.

gRNA spacers were cloned into expression backbones by Golden Gate assembly as previously described17. Spacer sequences are listed in Tables 23, 24, 26 and 27.

### Bacterial RNA Sequencing

Bacterial RNA sequencing was performed as previously described (18). Briefly, 5 mL overnight cultures of a Stbl3 E. coli colony transformed with a plasmid containing the locus of interest was spun down and resuspended in 1 mL of TRI Reagent (Zymo Research). After a 5-minute room temperature incubation, 250 μL of 0.5 mm Zirconia beads were added and the Trizol resuspension was vortexed vigorously for 30 s to 1 min. 200 uL chloroform was added, samples were inverted gently, incubated at room temperature for 3 minutes, and then spun down at 12000×g for 5 min at 4° C. Following centrifugation, the aqueous fraction was used as input to the Qiagen miRNeasy kit, as per the manufacturer's instructions.

Purified RNA was treated with DNase I (NEB), purified again using RNA Clean & Concentrate-25 (Zymo Research), and treated with T4 polynucleotide kinase (PNK) (NEB). PNK-treated RNA was again purified using RNA Clean & Concentrate-25 (Zymo Research), and ribosomal RNA was removed using the Ribominus Transcriptome Isolation Kit (Yeast and Bacteria) (Thermo Fisher Scientific). Samples were subsequently treated with RNA 5′ polyphosphatase (Epicentre) and purified again using an RNA Clean & Concentrate-5 kit (Zymo Research). Purified RNA was used as input to the NEBNext Multiplex Small RNA Library Prep Set for Illumina (NEB). Library preparation was performed as per the manufacturer's instructions, except with a final PCR of 20 cycles. Libraries were quantified by qPCR using the KAPA Library Quantification Kit for Illumina (Roche) on a StepOnePlus Real-Time PCR System (Thermo Fisher Scientific) and sequenced on an Illumina NextSeq. Reads were mapped using BWA and a custom Python script available upon request.

E. coli Essential Gene PFS Screen

Libraries were designed as previously described (6). The library of spacers was cloned into each Cas13b-t pJ23119-spacer-DR backbone containing a chloramphenicol resistance gene using Golden Gate Assembly with a 5:1 ratio of spacer library to pre-digested backbone with 210 cycles. Libraries were transformed into Endura Electrocompetent Cells (Lucigen) by electroporation and plated over five 22.7 cm×22.7 cm chloramphenicol LB agar plates. 12 hours after plating, libraries were scraped from plates and DNA was extracted using the Macherey-Nagel Nucleobond Xtra Maxiprep Kit (Macherey-Nagel). 200 ng of library plasmid and 200 ng Cas13b-t gene plasmid containing an ampicillin resistance gene were transformed into 100 μL of Endura Electrocompetent Cells (Lucigen) by electroporation as per the manufacturer's protocol and plated across four 22.7 cm×22.7 cm ampicillin/chloramphenicol LB agar plates per biological replicate, with three biological replicates per condition. 10-12 hours post-transformation, libraries of transformants were scraped from the plates and DNA was extracted using the Macherey-Nagel Nucleobond Xtra Maxiprep Kit (Macherey-Nagel). Libraries were prepared from extracted DNA for next generation sequencing using primers in Table 22 with NEBNext High-Fidelity 2X PCR Master Mix (NEB) and sequenced on an Illumina NextSeq. Spacer abundance relative to an empty vector was analyzed using a custom Python script, available on request. A mixed Gaussian distribution was fit to the distribution of negative control spacers, and the distribution with the higher mean was used as the null distribution. Depleted spacers were selected as those greater than 5 standard deviations away from the selected null distribution mean. Weblogos were generated using https://weblogo.berkeley.edu/logo.cgi using the top 1% of depleted spacers.

### Mammalian Cell Culture and Transfection

Mammalian cell culture experiments were performed in the HEK293FT line (American Type Culture Collection (ATCC)) grown in Dulbecco's Modified Eagle Medium with high glucose, sodium pyruvate, and GlutaMAX (Thermo Fisher Scientific), additionally supplemented with 1×penicillin-streptomycin (Thermo Fisher Scientific), 10 mM HEPES (Thermo Fisher Scientific), and 10% fetal bovine serum (VWR Seradigm). All cells were maintained at confluency below 80%.

All transfections were performed with Lipofectamine 2000 (Thermo Fisher Scientific) in 96-well plates. Cells were plated at approximately 20,000 cells/well 16-20 hours prior to transfection to ensure 90% confluency at the time of transfection. For each well on the plate, transfection plasmids were combined with OptiMEM I Reduced Serum Medium (Thermo Fisher Scientific) to a total of 25 μl. Separately, 24.5 μl of OptiMEM was combined with 0.5 μl of Lipofectamine 2000. Plasmid and Lipofectamine solutions were then combined and pipetted onto cells.

### Mammalian RNA Knockdown Assays

HEK293FT cells were transfected as described with 75 ng of a plasmid encoding expression of either a Cas13b-t ortholog or GFP from a CMV promoter, 150 ng of a plasmid encoding expression of a gRNA from a human U6 promoter and, where relevant, 45 ng of reporter plasmid. After 48 h, RNA was harvested as described previously17 with 2× the amount of recommended DNase and a 20 minute lysis step. RNA expression was measured by qPCR using commercially available TaqMan probes (Thermo Fisher Scientific) (Table 29) on a LightCycler 480 II (Roche) with GAPDH as an endogenous internal control in 5 uL multiplexed reactions (17). Probes and primer sets were generally selected to amplify across the Cas13 target site so as to minimize detection of cleaved transcripts. Data is the average of 4 biological replicates with fold-change calculated relative to a negative control condition with the corresponding gRNA expression plasmid co-transfected with the GFP expression plasmid rather than a Cas13b-t expression plasmid using the ddCt method (19). Error bars were calculated in GraphPad Prism 7 and represent the standard deviation, n=4.

For luciferase reporter assays, media was aspirated from cells and Cypridina and Gaussia luciferase activity in the media was measured using Gaussia and Cypridina Luciferase Assay Kits (Targeting Systems) with an injection protocol on a Biotek Synergy Neo 2 (Agilent). Each experimental luciferase measurement was normalized to the appropriate control luciferase measurement (i.e., if Cypridina luciferase was targeted, the Gaussia luciferase measurement was used as the control value and vice versa). For knockdown assays, normalized luciferase values were then again normalized to an average normalized luciferase measurement of 4 biological replicates of a negative control condition consisting of the corresponding gRNA expression plasmid co-transfected with a GFP expression plasmid rather than a Cas13 expression plasmid. Error bars were calculated in GraphPad Prism 7 and represent the standard deviation of the luciferase values normalized to negative control transfection, n=4.

### Mammalian RNA Editing Assays

HEK293FT cells were transfected as described with 150 ng a plasmid encoding expression of a dCas13b ortholog-ADAR2dd(E488Q) fusion from a CMV promoter, 300 ng of a plasmid encoding expression of a gRNA from a human U6 promoter and, where relevant, 45 ng of a reporter plasmid. After 48 h, RNA was harvested as described previously and reverse transcription was performed as described (17) using gene-specific primers for the relevant target transcript (Table 32). cDNA was used as input for library preparation of next-generation sequencing libraries (Table 33) using NEBNext High-Fidelity 2X PCR Master Mix (NEB), and amplicons were sequenced on an Illumina MiSeq. Editing was quantified by counting the number of reads at which the expected edited position in the amplicon was called as a G (for A-to-I editing) or T (for C-to-U editing) and dividing by the total number of reads in the sample using a custom Python script, available upon request. Unless otherwise noted, all reported data is the average of 4 biological replicates.

Luciferase reporter assays for RNA editing were performed as described above, with the modification that normalized luciferase values were not normalized to a GFP control condition. For CTNNB1 targeting, Applicants engineered a luciferase reporter by replacing the EF1alpha promoter driving Gaussia luciferase expression in the dual luciferase reporter plasmid with a promoter derived either from an M50 Super 8× TOPFlash (TOP) or M51 Super 8× FOPFlash (FOP) reporter. M50 Super 8× TOPFlash (Addgene plasmid #12456) and M51 Super 8× FOPFlash (TOPFlash mutant) (Addgene plasmid #12457) were gifts from Randall Moon3,20. Luciferase activity was measured for these custom dual luciferase reporters for each protein/gRNA condition and normalized as described for a dual luciferase reporter. Fold activation was calculated by taking the ratio of the average TOP measurement and dividing by average FOP measurement, and error was calculated by a standard error propagation formula.

Optimal spacers for all target sites tested were determined by tiling spacers across the site of interest, varying the distance of the mismatch from the DR from 14 bp to 28 bp in intervals of 2 bp.

### RNA Editing Specificity

HEK293FT cells were transfected as described for mammalian RNA editing assays. After 48 h, RNA was harvested using a QIAGEN RNeasy Plus 96 kit as per the manufacturer's protocol. The mRNA fraction was enriched using an NEBNext Poly(A) Magnetic Isolation Module (NEB). Libraries were prepared using an NEBNExt Ultra II Directional RNA library prep kit (NEB) as per the manufacturer's protocol and sequenced on an Illumina NextSeq. Each sample was sequenced with an average read depth of 8 million reads per sample and randomly downsampled to 5 million reads per sample. Data was analyzed using a previously described custom pipeline on the FireCloud computational framework and downstream analysis using a custom Python script2,3. Any significant edits found in eGFP-transfected conditions were considered to be SNPs or artifacts of the transfection and filtered out. An additional layer of filtering for known SNP positions was performed using the Kaviar2l method for identifying SNPs.

### REFERENCES

1. Terns, M. P. Mol. Cell 72, 404-412 (2018).
2. Cox, D. B. T. et al. Science 358, 1019-1027 (2017).
3. Abudayyeh, O. O. et al. Science 365, 382-386 (2019).
4. Dong, J. Y., Fan, P. D. & Frizzell, R. A. Hum. Gene Ther. 7, 2101-2112 (1996).
5. Wu, Z., Yang, H. & Colosi, P. Mol. Ther. 18, 80-86 (2010).
6. Smargon, A. A. et al. Mol. Cell 65, 618-630.e7 (2017).
7. Matthews, M. M. et al. Nat. Struct. Mol. Biol. 23, 426-433 (2016).
8. MacDonald, B. T., Tamai, K. & He, X. Developmental Cell vol. 17 9-26 (2009).
9. Apte, U. et al. Am. J. Pathol. 175, 1056-1065 (2009).
10. Bhushan, B. et al. Am. J. Pathol. 184, 3013-3025 (2014).
11. Shmakov, S. A., Makarova, K. S., Wolf, Y. I., Severinov, K. V. & Koonin, E. V. Proc. Natl. Acad. Sci. U.S.A 115, E5307-E5316 (2018).
12. Eddy, S. R. PLoS Comput. Biol. 7, e1002195 (2011).
13. Steinegger, M. & Söding, J. Nat. Biotechnol. 35, 1026-1028 (2017).
14. Steinegger, M. & Söding, J. Nat. Commun. 9, 2542 (2018).
15. Katoh, K. & Standley, D. M. Mol. Biol. Evol. 30, 772-780 (2013).
16. Makarova, K. S. et al. Nature Reviews Microbiology vol. 18 67-83 (2020).
17. Joung, J. et al. Nat. Protoc. 12, 828-863 (2017).
18. Zetsche, B. et al. Cell 163, 759-771 (2015).
19. Schmittgen, T. D. & Livak, K. J. Nature Protocols vol. 3 1101-1108 (2008).
20. Veeman, M. T., Slusarski, D. C., Kaykas, A., Louie, S. H. & Moon, R. T. Curr. Biol. 13, 680-685 (2003).
21. Glusman, G., Caballero, J., Mauldin, D. E., Hood, L. & Roach, J. C. Bioinformatics 27, 3216-3217 (2011).

### Supplementary Methods

### Design and Cloning of Yeast Expression Plasmid Constructs

Yeast reporter constructs were cloned into a pYES3/CT backbone (Thermo Fisher). A previously described reporter containing a crRNA expression cassette under a pADH1 terminator (1) was digested with HindIII and MluI (Thermo Fisher). A URA3 gene was amplified by PCR using the selection marker from a pRSII426 backbone (2) with the introduced stop codon added by site-directed mutagenesis (Table 24) and cloned via Gibson assembly This backbone was digested with BcuI (Thermo Fisher) and an ADE2 gene amplified from M3499 ura3::ADE2 Disruptor Converter (Addgene plasmid #51674) (3), with the introduced stop codon added by site-directed mutagenesis (Table 24) and cloned via Gibson assembly. gRNA spacers were cloned into this backbone using Golden Gate assembly (4). Final constructs are listed in Table 31.

Yeast REPAIR expression plasmids were derived from a previously described pRSII426 backbone (2) with a pGAL promoter driving expression of the REPAIR fusion protein (2). The URA3 selection marker was replaced with a LEU2 selection marker by digesting this backbone with Eco105I and KpnI (Thermo Fisher) and inserting a LEU2 gene amplified from a synthesized gene (IDT) by Gibson assembly. ADAR2 mutants to create sequences that could be used as a basis for error-prone PCR for each subsequent evolution round were inserted by amplifying the analogous sequence from the previous round of evolution and adding the new mutation via the site-directed mutagenesis (Table 24). Final constructs are listed in Table 31.

### Cloning of Mutagenesis Libraries for ADAR Evolution

ADAR2dd mutant libraries were generated by performing 8 error-prone PCR reactions for 20 cycles using a GeneMorph II Random Mutagenesis Kit (Agilent) with titrated template concentrations. For each round of evolution, Applicants used a yeast codon-optimized ADAR2dd gene containing the selected mutants from all prior rounds. Resulting PCR reactions were pooled, gel purified, subjected to DpnI (Thermo Fisher) treatment and cloned into a yeast RanCas13b-REPAIR expression backbone (Table 35) digested with KflI and Eco72I (Thermo Fisher) by Gibson assembly. Libraries were transformed into Endura Electrocompetent Cells (Lucigen) by electroporation and plated over one 22.7 cm×22.7 cm ampicillin LB agar plate. After 12-16 hours of growth, libraries were scraped from plates and DNA was extracted using the Macherey-Nagel Nucleobond Xtra Maxiprep Kit (Macherey-Nagel). Primers are listed in Table 20.

### Directed Evolution of High-Specificity ADAR Mutants

Applicants performed two rounds of evolution as follows: To select for highly specific and efficient ADAR variants, Applicants engineered a yeast reporter based on simultaneous restoration of a TGA stop codon in ADE2 and negative selection of restoration of a TAG stop codon in URA3. Applicants transformed Saccharomyces cerevisiae Meyen ex E. C. Hansen (ATCC 204681) with this plasmid, which also included expression of a crRNA targeting ADE2. Yeast were transformed using the lithium acetate/single-stranded carrier DNA/PEG method (5).

Large scale transformations of mutagenesis libraries were performed as previously described (1, 6). Briefly, Applicants picked a colony from the initial transformation of the reporter plasmid, inoculated 300 mL of 2% glucose minimal media -tryptophan (Trp) for selection and grew overnight in a baffled flask at 30° C. After 12-16 hours of growth, Applicants measured the optical density (OD) of the culture and used this measurement to seed 2.5E9 cells into 500 mL of pre-warmed 2×YPAD media in a non-baffled flask. Once this culture reached an OD of 2 (approximately 4 hours), cells were harvested by centrifugation at 3000×g for 5 min, followed by two washes with water. The resulting cell pellet was then resuspended in 36 mL of transformation mix consisting of 24 mL of PEG 3350 (50% w/v), 3.6 mL of 1.0 M Lithium acetate, 5 mL of denatured single-stranded carrier salmon sperm DNA at 2.0 mg/mL (Thermo Fisher), 2.9 mL of water, and 500 μL of 1 μg/μL plasmid library. The mixture was incubated at 42° C. for 60 minutes with agitation, then the cells were pelleted once more and resuspended in 750 mL of 2% glucose minimal media -Trp/-leucine (Leu) and grown overnight at 30° C. in a baffled flask until OD reached between 6 and 8. 6.25 mL of the culture was then seeded into 250 mL of 2% raffinose -Trp/-Leu selection media and grown until OD reached between 0.5 and 1. The culture was then induced by adding 27 mL of 30% galactose and incubated overnight at 30° C. for 12-15 hours.

After overnight growth, cultures were plated across 20 22.7×22.7 cm selection plates of 2% raffinose/3% galactose -Trp/-Leu with 5 mg/L adenine (Ade) and 0.1% 5-fluoroorotic acid (5-FOA). After 2-3 days of selection, we picked white colonies corresponding to an on-target edit and restoration of ADE2 and streaked these onto small selection plates of the same media base to ensure accurate colony picking. Plates were then allowed to grow again for up to 3 days. White streaks after this second selection were again picked.

To look for enriched single mutations, all picked streaks were pooled and the contained RanCas13b-REPAIR genes were amplified with NEBNext High-Fidelity 2X PCR Master Mix (NEB) for preparation of next generation sequencing libraries. Libraries were sequenced on an Illumina NextSeq. Primers for library amplification are found in Table 30. Relative enrichment of mutations in the selected library was analyzed using a custom Python script, available upon request. Identified enriched single mutants were introduced by site-directed mutagenesis to RanCas13b-REPAIR in mammalian expression vectors for validation (Table 21).

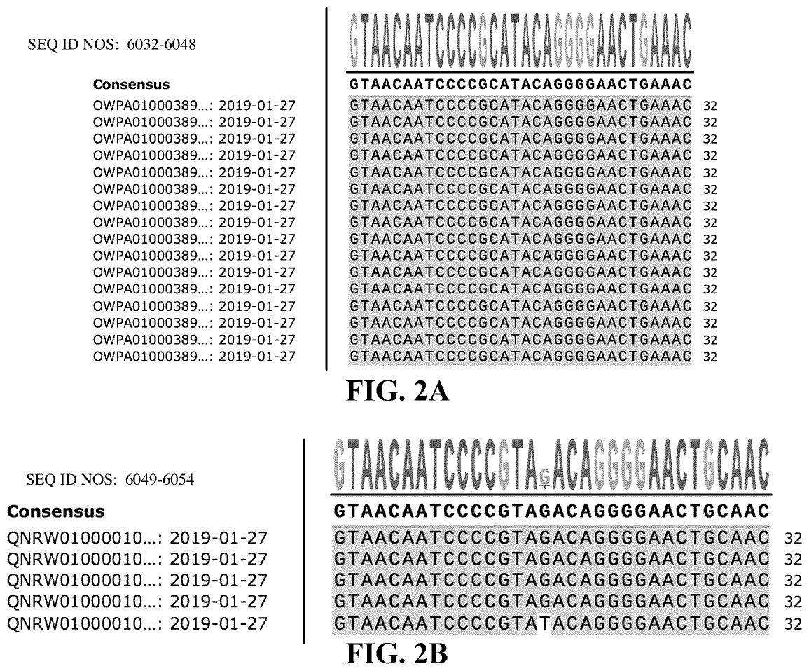

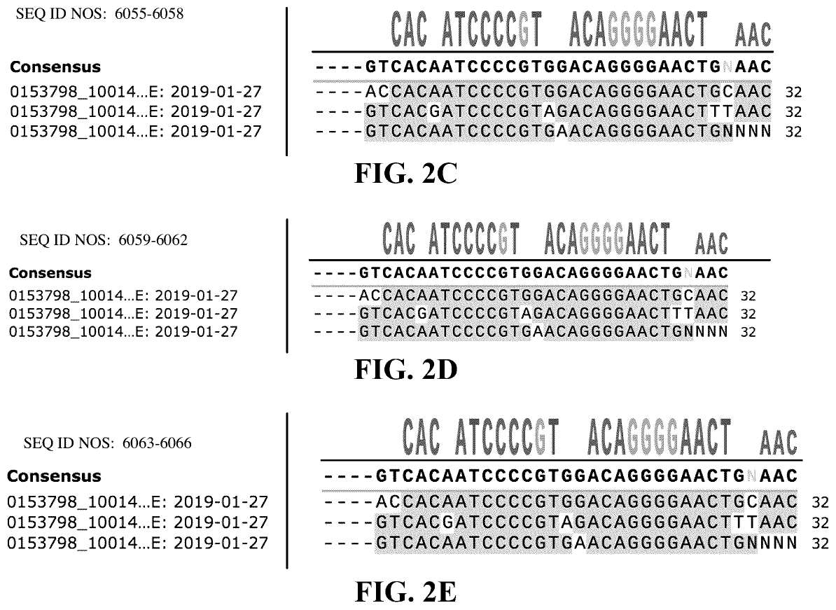

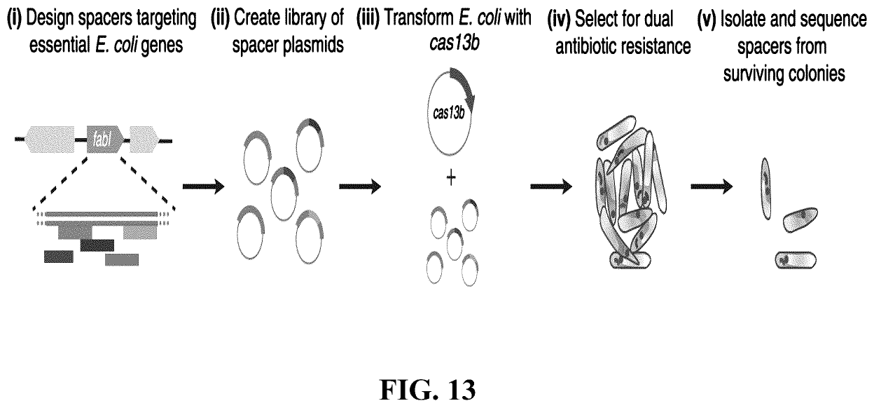

To test the candidate mutations, RNA editing assays using luciferase reporters in HEK293FT cells were performed as previously described. Specifically, after the first round of selection, RanCas13b-ADAR2dd mutants were targeted to either of 2 Cypridina luciferase reporters, one with a W85X mutation (TAG stop codon) and one with a W13X mutation (TGA stop codon) to evaluate the ability of the evolved ADAR2dd's to effectively edit at sites with both preferred and non-preferred 5′ bases (7, 8) (FIGS. 25A-25B). After the second round of evolution, this initial screening was performed using the same Cypridina luciferase W85X reporter, along with a second Cypridina luciferase W85X (TGA stop codon) reporter and a Gaussia luciferase R93H reporter for which restoration of a CAT codon to CGT reverts a catalytically-inactivating mutation (FIGS. 2A-26C). Luciferase activity of the Cypridina luciferase W85X TAG reporter in the non-targeting crRNA condition was also used as a proxy for measuring specificity, as previously described (9).

Based on this initial screening pass, top candidates were further validated for broad activity by testing again on the initial screen sites and additionally targeting the K19 and H36 codons in the endogenous CTNNB1 transcript after the first round of selection (FIGS. 28C-28F), and additionally on Gaussia luciferase reporters with G92R, R93K and R93Q catalytic mutations as well as the targeting of the T41 codon in CTNNB1 (FIGS. 29D-29J). Based on activity at all tested sites as measured by either next-generation sequencing and luciferase assays, as well as specificity measured as described, a single top candidate was identified and cloned into the RanCas13b-REPAIR yeast expression construct derived from the previous round of evolution to use as a basis for mutagenesis for the subsequent round.

After Round 1, Applicants identified the E620G mutation and after Round 2, we identified the Q696L mutation. Applicants additionally identified V505I as a mutation capable of enhancing editing at target sites with a s (FIGS. 29A-29J).

TABLE 15
Accessions of contigs containing Cas13b-t orthologs

| | | | | Sample |
|---|---|---|---|---|
| | | | | collection |
| Source | | Ortholog name | | temperature |
| database | Contig accession | (if applicable) | Source habitat/organism | (C.) |
| JGI | Ga0246100_107590 | | Groundwater | |
| JG | Ga0265293_10004442 | | Landfill leachate | |
| JGI | Ga0315543_1000530 | | Salt marsh sediment | |
| JGI | Ga0209381_1018281 | Cas13b-t5 | Hot spring sediment | 64.7 |
| JGI | Ga0310137_000061 | | Fracking water | |
| JGI | Ga0208824_1000897 | | Anaerobic digester sludge | |
| JGI | Ga0137489_1004561 | | Basal ice | |
| JGI | Ga0315552_1001799 | | Salt marsh sediment | |
| JGI | Ga0180434_10014215 | Cas13b-t4 | Hypersaline lake sediment | |
| JGI | Ga0315532_1010951 | | Salt marsh sediment | |
| JGI | Ga0307431_1000754 | | Salt marsh sediment | |
| JGI | Ga0315541_1003536 | | Salt marsh sediment | |
| JGI | Ga0315296_10033793 | | Freshwater lake sediment | |
| JGI | Ga0307443_1009138 | | Salt marsh sediment | |
| JGI | Ga0315532_1006943 | | Salt marsh sediment | |
| JGI | Ga0315554_1005387 | | Salt marsh sediment | |
| NCBI WGS | QNBS01000103.1 | Cas13b-t3 | Planctomycetes bacterium | |
| | | | isolate B28 G16 (marine | |
| | | | sediment) | |
| JGI | Ga0315285_10018775 | | Freshwater lake sediment | |
| JGI | Ga0315294_10038294 | | Freshwater lake sediment | |
| JGI | Ga0315533_1000464 | | Salt marsh sediment | |
| JGI | Ga0209427_10000033 | Cas13b-t2 | Marine sediment | |
| JGI | Ga0114919_10002421 | Cas13b-t1 | Atlantic deep subsurface | |

JGI: Joint Genome Institute
NCBI WGS: National Center for Biotechnology Information Whole Genome Shotgun

TABLE 16
Direct repeat sequences of Cas13 orthologs
used in this study

| | Abbrevi- | |
|---|---|---|
| | ation | |
| Organism | key | DR sequence |
| Riemerella | Ran | GTTGGGACTGCTCTCACTTT |
| anatipestifer | | GAAGGGTATTCACAAC |
| | | (SEQ ID NO: 5266) |
| Prevotella | Psp | GTTGTGGAAGGTCCAGTTTT |
| sp. P5-125 | | GAGGGGCTATTACAAC |
| | | (SEQ ID NO: 5267) |
| | b-t1 | GCTGTAATCACCCCACAAAT |
| | | CGGAGGCTTCTTCAGC |
| | | (SEQ ID NO: 5268) |
| | b-t2 | GCTGTAATCACCCCACAAAT |
| | | CGGGGGCTTCTCCAGC |
| | | (SEQ ID NO: 5269) |
| | b-t3 | GCTGTAATCACCCCACAAAT |
| | | CGGGGGCTGCTCCAGC |
| | | (SEQ ID NO: 5270) |
| | b-t4 | GCTGTTACTTCCCCACAAAT |
| | | TGAGGCCCATCACAGC |
| | | (SEQ ID NO: 5271) |
| | b-t5 | GCTGTGATTACCCTGCAAAT |
| | | CGAGGGCTGCTCCAGC |
| | | (SEQ ID NO: 5272) |

TABLE 17
Cas13 orthologs used in this study

| Abbreviation | |
|---|---|
| key | Protein sequence |
| Ran | MEKPLLPNVYTLKHKFFWGAFLNIARHNAFITICHINEQLGLKTPSNDDKIVDVVCETWNNILNNDHDLL |
| | KKSQLTELILKHFPFLTAMCYHPPKKEGKKKGHQKEQQKEKESEAQSQAEALNPSKLIEALEILVNQLHS |
| | LRNYYSHYKHKKPDAEKDIFKHLYKAFDASLRMVKEDYKAHFTVNLTRDFAHLNRKGKNKQDNPDFN |
| | RYRFEKDGFFTESGLLFFTNLFLDKRDAYWMLKKVSGFKASHKQREKMTTEVFCRSRILLPKLRLESRY |
| | DHNQMLLDMLSELSRCPKLLYEKLSEENKKHFQVEADGFLDEIEEEQNPFKDTLIRHQDRFPYFALRYLD |
| | LNESFKSIRFQVDLGTYHYCIYDKKIGDEQEKRHLTRTLLSFGRLQDFTEINRPQEWKALTKDLDYKETS |
| | NQPFISKTTPHYHITONKIGFRLGTSKELYPSLEIKDGANRIAKYPYNSGFVAHAFISVHELLPLMFYQHLT |
| | GKSEDLLKETVRHIQRIYKDFEEERINTIEDLEKANQGRLPLGAFPKQMLGLLQNKQPDLSEKAKIKIEKLI |
| | AETKLLSHRLNTKLKSSPKLGKRREKLIKTGVLADWLVKDFMRFQPVAYDAQNQPIKSSKANSTEFWFI |
| | RRALALYGGEKNRLEGYFKQTNLIGNTNPHPFLNKFNWKACRNLVDFYQQYLEQREKFLEAIKNQPWE |
| | PYQYCLLLKIPKENRKNLVKGWEQGGISLPRGLFTEAIRETLSEDLMLSKPIRKEIKKHGRVGFISRAITLY |
| | FKEKYQDKHQSFYNLSYKLEAKAPLLKREEHYEYWQQNKPQSPTESQRLELHTSDRWKDYLLYKRWQ |
| | HLEKKLRLYRNQDVMLWLMTLELTKNHFKELNLNYHQLKLENLAVNVQEADAKLNPLNQTLPMVLPV |
| | KVYPATAFGEVQYHKTPIRTVYIREEHTKALKMGNFKALVKDRRLNGLFSFIKEENDTQKHPISQLRLRR |
| | ELEIYQSLRVDAFKETLSLEEKLLNKHTSLSSLENEFRALLEEWKKEYAASSMVTDEHIAFIASVRNAFCH |
| | NQYPFYKEALHAPIPLFTVAQPTTEEKDGLGIAEALLKVLREYCEIVKSQI (SEQ ID NO: 5273) |
| Psp | MNIPALVENQKKYFGTYSVMAMLNAQTVLDHIQKVADIEGEQNENNENLWFHPVMSHLYNAKNGYDK |
| | QPEKTMFIIERLQSYFPFLKIMAENQREYSNGKYKQNRVEVNSNDIFEVLKRAFGVLKMYRDLTNHYKT |
| | YEEKLNDGCEFLTSTEQPLSGMINNYYTVALRNMNERYGYKTEDLAFIQDKRFKFVKDAYGKKKSQVN |
| | TGFFLSLQDYNGDTQKKLHLSGVGIALLICLFLDKQYINIFLSRLPIFSSYNAQSEERRIIIRSFGINSIKLPKD |
| | RIHSEKSNKSVAMDMLNEVKRCPDELFTTLSAEKQSRFRIISDDHNEVLMKRSSDRFVPLLLQYIDYGKL |
| | FDHIRFHVNMGKLRYLLKADKTCIDGQTRVRVIEQPLNGFGRLEEAETMRKQENGTFGNSGIRIRDFEN |
| | MKRDDANPANYPYIVDTYTHYILENNKVEMFINDKEDSAPLLPVIEDDRYVVKTIPSCRMSTLEIPAMAF |
| | HMFLFGSKKTEKLIVDVHNRYKRLFQAMQKEEVTAENIASFGIAESDLPQKILDLISGNAHGKDVDAFIR |
| | LTVDDMLTDTERRIKRFKDDRKSIRSADNKMGKRGFKQISTGKLADFLAKDIVLFQPSVNDGENKITGLN |
| | YRIMQSAIAVYDSGDDYEAKQQFKLMFEKARLIGKGTTEPHPFLYKVFARSIPANAVEFYERYLIERKFY |
| | LTGLSNEIKKGNRVDVPFIRRDQNKWKTPAMKTLGRIYSEDLPVELPRQMFDNEIKSHLKSLPQMEGIDF |
| | NNANVTYLIAEYMKRVLDDDFQTFYQWNRNYRYMDMLKGEYDRKGSLQHCFTSVEEREGLWKERAS |
| | RTERYRKQASNKIRSNRQMRNASSEEIETILDKRLSNSRNEYQKSEKVIRRYRVQDALLFLLAKKTLTEL |
| | ADFDGERFKLKEIMPDAEKGILSEIMPMSFTFEKGGKKYTITSEGMKLKNYGDFFVLASDKRIGNLLELV |
| | GSDIVSKEDIMEEFNKYDQCRPEISSIVFNLEKWAFDTYPELSARVDREEKVDFKSILKILLNNKNINKEQS |
| | DILRKIRNAFDHNNYPDKGVVEIKALPEIAMSIKKAFGEYAIMK (SEQ ID NO: 5274) |
| b-t1 | MEFENIKKTSNKEVYSIEQYEGEKKWCFAIVLNRAQTNLEENPKLFEQTLTRFEKIMKQDWFNEETKKLIY |
| | EKEEENKVKEEIQIAASERLKNLANYFSAYLHAPDCLIFNRNDTIRIIMEKAYEKSRFEAKKKQQEDISIEFP |
| | ELFEEEDKITSAGVVFFVSFFIERRFLNRLMGYVQGFRKTEGEYNITRQVFSKYCLKDSYSVQAQDHDAVM |
| | FRDILGYLSRVPTEIYQHIKLTRKRSQDQLSERKTDKFILFALKYLEDYGLKDLADYTACFARSKIKRENED |
| | TKETDGNKHKFHREKPVVEIHFDKEKQDQFYIKRNNVILKAQKKGGQSNVFRMGVYELKYLVLLSLLGK |
| | AEEAIQRIDRYISSLKKQLPYLDKISNEEIQKSINFLPRFVRSRLGLLQVDDEKRLKTRLEYVKAKWTDKKE |
| | GSRKLELHRKGRDILRYINERCDRPLSRKEYNNILKFIVNKDFAGFYNELEELKRTRRLDKNIIQKLSGHTTL |
| | NALHERVCDLVLQELGSLQSENLKEYIGLIPKEEKEVTFREKVDRILEQPVVYKGFLRYEFFKEDKKSFARL |
| | VEEAIKTKWSDFDIPLGEEYYNIPSLDRFDRTNKKLYETLAMDRLCLMMARQYYLRLNEKLAEKAQHIYW |
| | KKEDGREVIIFKFQNPKEQKKSFSIRFSILDYTKMYVMDDPEFLSRLWEYFIPKEAKEIDYHKHYARAFDKY |
| | TNLQKEGIDAILKLEGRIIERRKIKPAKNYIEFQEIMNRSGYNNDQQVALKRVANALLAYNLNFEREHLKRF |
| | YGVVKREGIEKKWSLIV (SEQ ID NO: 5275) |
| b-t2 | MQVENIKKGSSQGMYSIEQYEGAKKWCFAIVLNRAQTNLQGNPKLFEETLTRFERIRKEDWFDQETKKLI |
| | YAKQEQNEVEEEIQKAADEKLRDLRNYFSHYFHTPDCLIFTQNDPVRIIMEKAYEKARFEQAKKEQEDISIE |
| | FGELFEENGRITSAGVVFFASFFAERRFLNRLMGYVQGFTRTEGEYKITRDVFSTYCLRDSYSVKTPDHDA |
| | VMFRDILGYLSRVPSESYQRIKESQMRSETQLSERKTDKFILFALNYLEDYGLEDLADYTACFARTRIKREQ |
| | DENTOGKEQKPHRKKPRVEIHFERAEGDPFYIKHNNVILRTQKKGAQTYIFRMGVYELKYLVLLSLLGKG |
| | AEAVKRIDRYVHSLRNQLPHIEKKSTEEIEGYVRFLPRFVRSHLGLLGVDDEKKIKARVDYVKAKWLEKK |
| | EKSRELQLHRKGRDILRYINERCERPLNIDEYNRILELLVTKHLDGFYRELEELKKTRRIDKNIVCNLSRHKS |
| | VNALHEKVCDLVVQELESLGREELKEYVGLIPKEEKEVSFEEKTDRVVKQPVIYKGFLRNEFFRESRKSFA |
| | RLVEEAVREKGEVYDVPLGGEYYEIVSLDTFDKDNKRLYETLAMDRLLLMIARQYHLSLNKELAKRAQQI |
| | EWKKEDGEEVIIFTLKNPAQPEQSCSVRFSLRDYTKLYVMDDAEFLARLCDYFLPKDEEQIDYHRLYTQG |
| | MNRYTNLQREGIEAILELEKKTIGPEQPRPPKNYIPFSEIMDKSAYNEDDQKALRRVRNALLHHNLNFARA |
| | DFKRFCGIMKREGIEKRWSLAV (SEQ ID NO: 5276) |
| b-t3 | MAQVSKQTSKKRELSIDEYQGARKWCFTIAFNKALVNRDKNDGLFVESLLRHEKYSKHDWYDEDTRALI |
| | KCSTQAANAKAEALANYFSAYRHSPGCLTFTAEDELRTIMERAYERAIFECRRRETEVIIEFPSLFEGDRITT |
| | AGVVFFVSFFVERRVLDRLYGAVSGLKKNEGQYKLTRKALSMYCLKDSRFTKAWDKRVLLFRDILAQLG |
| | RIPAEAYEYYHGEQGDKKRANDNEGTNPKRHKDKFIEFALHYLEAQHSEICFGRRHIVREEAGAGDEHKK |
| | HRTKGKVVVDFSKKDEDQSYYISKNNVIVRIDKNAGPRSYRMGLNELKYLVLLSLQGKGDDAIAKLYRY |
| | RQHVENILDVVKVTDKDNHVFLPRFVLEQHGIGRKAFKQRIDGRVKHVRGVWEKKKAATNEMTLHEKA |
| | RDILQYVNENCTRSFNPGEYNRLLVCLVGKDVENFQAGLKRLQLAERIDGRVYSIFAQTSTINEMHQVVC |
| | DQILNRLCRIGDQKLYDYVGLGKKDEIDYKQKVAWFKEHISIRRGFLRKKFWYDSKKGFAKLVEEHLESG |
| | GGQRDVGLDKKYYHIDAIGRFEGANPALYETLARDRLCLMMAQYFLGSVRKELGNKIVWSNDSIELPVEG |
| | SVGNEKSIVFSVSDYGKLYVLDDAEFLGRICEYFMPHEKGKIRYHTVYEKGFRAYNDLQKKCVEAVLAFE |
| | EKVVKAKKMSEKEGAHYIDFREILAQTMCKEAEKTAVNKVARAFFAHHLKFVIDEFGLFSDVMKKYGIE |
| | KEWKFPVK (SEQ ID NO: 5277) |
| b-t4 | MNIIKLKKEEAAFYFNQTILNLSGLDEIIEKQIPHIISNKENAKKVIDKIFNNRLLLKSVENYIYNFKDVAKNA |
| | RTEIEAILLKLVELRNFYSHYVHNDTVKILSNGEKPILEKYYQIAIEATGSKNVKLVIIENNNCLTOSGVLFL |
| | LCMFLKKSQANKLISSVSGFKRNDKEGQPRRNLFTYYSVREGYKVVPDMQKHFLLFALVNHLSEQDDHIE |
| | KQQQSDELGKGLFFHRIASTFLNESGIFNKMQFYTYQSNRLKEKRGELKHEKDTFTWIEPFQGNSYFTLNG |
| | HKGVISEDQLKELCYTILIEKQNVDSLEGKIIQFLKKFQNVSSKQQVDEDELLKREYFPANYFGRAGTGTLK |
| | EKILNRLDKRMDPTSKVTDKAYDKMIEVMEFINMCLPSDEKLRQKDYRRYLKMVRFWNKEKHNIKREFD |
| | SKKWTRFLPTELWNKRNLEEAYQLARKENKKKLEDMRNQVRSLKENDLEKYQQINYVNDLENLRLLSQE |
| | LGVKWQEKDWVEYSGQIKKQISDNQKLTIMKQRITAELKKMHGIENLNLRISIDTNKSRQTVMNRIALPKG |
| | FVKNHIQQNSSEKISKRIREDYCKIELSGKYEELSRQFFDKKNFDKMTLINGLCEKNKLIAFMVIYLLERLGF |
| | ELKEKTKLGELKQTRMTYKISDKVKEDIPLSYYPKLVYAMNRKYVDNIDSYAFAAYESKKAILDKVDIIEK |
| | QRMEFIKQVLCFEEYIFENRIIEKSKFNDEETHISFTQIHDELIKKGRDTEKLSKLKHARNKALHGEIPDGTSF |
| | EKAKLLINEIKK (SEQ ID NO: 5278) |
| b-t5 | MGIDYSLTSDCYRGINKSCFAVALNIAYDNCDHKGCRTLLSEVLRSKGGISDEQIKSQVVDGIQKRLKDIRN |
| | YFSHYYHAEDCLRFGDQDAVKVFLEEIYKNAESKTVGATKESDYKGVVPPLFELHNGTYMITAAGVIFLA |
| | SFFCHRSNVYRMLGAVKGFKHTGKEQLSDGQKRDYGFTRRLLAYYALRDSYSVGAEDKTRCFREILSYLS |
| | RVPQLAVDWLNEQQLLTPEEKEAFLNQPAEDEGGDISDSSSSDKNKKSKEKRRSLRRDEKFILFAIQFIEGW |
| | AAEQGLDVTFARYQKTVEKAENKNQDGKQARAVQLKYRNQGLNPDFNNEWMYYIQNEHAIIQIKLNNK |
| | KAVAARISENELKYLVLLIFEEKGNDAVQKLNCYIYSMSQKIEGEWKHRPEDERWMPSFTKRADRTVTPE |
| | AVQSRLSYIRKQLQETIEKIGQEEPRNNKWLIYKGKKISMILKFISDSIRDIQRRPNVKQYHILRDALQRLDF |
| | DGFYKELQNYVNDGRIAVSLYDQIKGVNDISGLCKKVCELTLERLAGLEAKNGSELRRYIGLEAQEKHPK |
| | YGEWNTLQEKAKRFLESQFSIGKNFLRKMFYGDCCQKRCFDEEKGYNTQAKERKSLYSIVKEKLKDIKPIH |
| | DDRWYLIDRNPKNYDNKHSRIIRQMCNTYIQDVLCMKMAMWHYEKLISATEFRNKLEWNCIGQGNMGY |
| | ERYSLWYKTGCGVVIQFTPADFLRLDIIEKPAMIENICQCFVLGNKKLNSGAEKKITWDKFNKDGIAKYRK |
| | RQAEAVRAIFAFEEGLKIQEDKWSHERYFPFCNILDEAVKQGKIKDTGKDKEALNRGRNDFFHEEFKSTED |
| | QQAIFQKYFPIVERKDDTKKRRDKKQK (SEQ ID NO: 5279) |

TABLE 18
Primers for cloning plasmids used in PFS screen

| Name | Sequence |
|---|---|
| Cas13b-t1_gene_F | TGCCGGGCCTCTTGCGGGATTTTACACTTTATGCTTCCGGCTCGTATGTTAGGAGGTC |
| | TTTATCATGGAATTCGAGAACATCAA (SEQ ID NO: 5280) |
| Cas13b-t1_gene_R | ATGCTGTCGGAATGGACGATTCACACGATCAGGGACCATT (SEQ ID NO: 5281) |
| Cas13b-t2_gene_F | TGCCGGGCCTCTTGCGGGATTTTACACTTTATGCTTCCGGCTCGTATGTTAGGAGGTC |
| | TTTATCATGCAGGTCGAGAACATCAA (SEQ ID NO: 5282) |
| Cas13b-t2_gene_R | ATGCTGTCGGAATGGACGATTCACACAGCCAGGGACCATC (SEQ ID NO: 5283) |
| Cas13b-t3_gene_F | TGCCGGGCCTCTTGCGGGATTTTACACTTTATGCTTCCGGCTCGTATGTTAGGAGGTC |
| | TTTATCATGGCCCAGGTGTCCAAGCA (SEQ ID NO: 5284) |
| Cas13b-t3_gene_R | ATGCTGTCGGAATGGACGATTCACTTCACGGGGAACTTCC (SEQ ID NO: 5285) |
| Cas13b-t4_gene_F | TGCCGGGCCTCTTGCGGGATTTTACACTTTATGCTTCCGGCTCGTATGTTAGGAGGTC |
| | TTTATCATGAACATCATCAAGCTGAA (SEQ ID NO: 5286) |
| Cas13b-t4_gene_R | ATGCTGTCGGAATGGACGATTTACTTCTTAATCTCATTGA (SEQ ID NO: 5287) |
| Cas13b-t5_gene_F | TGCCGGGCCTCTTGCGGGATTTTACACTTTATGCTTCCGGCTCGTATGTTAGGAGGTC |
| | TTTATCATGGGCATCGATTACAGCCT (SEQ ID NO: 5288) |
| Cas13b-t5_gene_R | ATGCTGTCGGAATGGACGATTTACTTCTGCTTCTTGTCTC (SEQ ID NO: 5289) |
| crRNA_expression_bac_F | TGCCGGGCCTCTTGCGGGATATCTTGACAGCTAGCTCAGTCCT (SEQ ID NO: 5290) |
| Cas13b-t1_crRNA_R | GCGTCCGGCGTAGAGGATCCGCTGTAATCACCCCACAAAT (SEQ ID NO: 5291) |
| Cas13b-t2_crRNA_R | GCGTCCGGCGTAGAGGATCCGCTGTAATCACCCCACAAAT (SEQ ID NO: 5292) |
| Cas13b-t3_crRNA_R | GCGTCCGGCGTAGAGGATCCGCTGTAATCACCCCACAAAT (SEQ ID NO: 5293) |
| Cas13b-t4_crRNA_R | GCGTCCGGCGTAGAGGATCCGCTGTTACTTCCCCACAAAT (SEQ ID NO: 5294) |
| Cas13b-t5_crRNA_R | GCGTCCGGCGTAGAGGATCCGCTGTGATTACCCTGCAAAT (SEQ ID NO: 5295) |

TABLE 19
Primers for cloning mammalian expression plasmids. Mutations
introduced by PCR are shown in lower case.

| Name | Sequence |
|---|---|
| crRNA_expression_mammalian_F | GACCGAGCGCAGCGAGTCAGTGAGCGAGGA (SEQ ID NO: 5296) |
| Cas13b-t1_crRNA_mammalian_R | AACGACGGCCAGTGAATTCGAGCTCGGTACCAAAAAAGCTGTAATCACCCC |
| | ACAAATCGGAGGCTTCTTCAGCTTGTCTTCGTCCCAGGAAGACATGGTGTTT |
| | CGTCCTTTCCACAAGATATATAAA (SEQ ID NO: 5297) |
| Cas13b-t3_crRNA_mammalian_R | AACGACGGCCAGTGAATTCGAGCTCGGTACCAAAAAAGCTGTAATCACCCC |
| | ACAAATCGGGGGCTGCTCCAGCTTGTCTTCGTCCCAGGAAGACATGGTGTTT |
| | CGTCCTTTCCACAAGATATATAAA (SEQ ID NO: 5298) |
| Cas13b-t5_crRNA_mammalian_R | AACGACGGCCAGTGAATTCGAGCTCGGTACCAAAAAAGCTGTGATTACCCT |
| | GCAAATCGAGGGCTGCTCCAGCTTGTCTTCGTCCCAGGAAGACATGGTGTTT |
| | CGTCCTTTCCACAAGATATATAAA (SEQ ID NO: 5299) |
| Cas13b-t1_gene_mammalian_F | GAGACCCAAGCTGGCTAGCGTTTAAACTTAAGCTTGCCACCATGGGATCCCT |
| | TCAACTGCCTCCACTTGAAAGACTGACACTGGGATCCGAATTCGAGAACATC |
| | AAGAAAA (SEQ ID NO: 5300) |
| Cas13b-t1_HEPN1_mut_R | CAGGTAGgcGCTGAAGTAGTTTgcCAGGTTC (SEQ ID NO: 5301) |
| Cas13b-t1_HEPN1_mut_F | GAACCTGgcAAACTACTTCAGCgcCTACCTG (SEQ ID NO: 5302) |
| Cas13b-t1_HEPN2_mut_R | GTTGTAGgcCAGCAGGGCGTTCgcCACTCTC (SEQ ID NO: 5303) |
| Cas13b-t1_HEPN2_mut_F | GAGAGTGgcGAACGCCCTGCTGgcCTACAAC (SEQ ID NO: 5304) |
| Cas13b-t1_forREPAIR_R | ACTACCGCCTGACCCTCCCACGATCAGGGACCATTTTTTCTCG (SEQ ID NO: |
| | 5305) |
| Cas13b-t3_gene_mammalian_F | GAGACCCAAGCTGGCTAGCGTTTAAACTTAAGCTTGCCACCATGGGATCCCT |
| | TCAACTGCCTCCACTTGAAAGACTGACACTGGGATCCGCCCAGGTGTCCAA |
| | GCAGACCA (SEQ ID NO: 5306) |
| Cas13b-t3_HEPN1_mut_R | TCTGTAGgCGCTGAAGTAGTTTgcCAGAGCC (SEQ ID NO: 5307) |
| Cas13b-t3_HEPN1_mut_F | GGCTCTGgcAAACTACTTCAGCgcCTACAGA (SEQ ID NO: 5308) |
| Cas13b-t3_HEPN2_mut_R | GTGGTGGgcAAAGAAGGCTCTCgcCACTTTG (SEQ ID NO: 5309) |
| Cas13b-t3_HEPN2_mut_F | CAAAGTGgcGAGAGCCTTCTTTgcCCACCAC (SEQ ID NO: 5310) |
| Cas13b-t3_forREPAIR_R | ACTACCGCCTGACCCTCCCTTCACGGGGAACTTCCATTCTTTC (SEQ ID NO: |
| | 5311) |
| Cas13b-t5_gene_mammalian_F | GAGACCCAAGCTGGCTAGCGTTTAAACTTAAGCTTGCCACCATGGGATCCCT |
| | TCAACTGCCTCCACTTGAAAGACTGACACTGGGATCCATGGGCATCGATTAC |
| | AGCCTGACCA (SEQ ID NO: 5312) |
| ADAR2_F | GGAGGGTCAGGCGGTAGTCAGCTGCATTTA (SEQ ID NO: 5313) |
| pcDNA_expression_R | GGGTTTAAACGGGCCCTCTAGACTC (SEQ ID NO: 5314) |

TABLE 20
Primers for cloning yeast constructs used in this study

| Name | Sequence |
|---|---|
| ADAR_mut_library_F | CCAGATCGGGGGTTCCGGCGGGTCC (SEQ ID NO: 5315) |
| ADAR_mut_library_R | TATTTAATAATAAAAATCATAAATCATAAGAAATTCGCCACGTGAGT |
| | CTAGGATCCTCA (SEQ ID NO: 5316) |
| URA3_F | ACTCACTATAGGGAATATTAAGCTTTTCAATTCATCATTTTTTTT (SEQ |
| | ID NO: 5317) |
| URA3_R | TTAGTTTTGCTGGCCGCATC (SEQ ID NO: 5318) |
| URA3_TAG_R | AATGTCTGCCTATTCTGCTAT (SEQ ID NO: 5319) |
| URA3_TAG_F | ATAGCAGAATAGGCAGACATT (SEQ ID NO: 5320) |
| ADE2_F | GGGCGCGTGGGGATGATCCATTCTTGAATAATACATAACT (SEQ ID |
| | NO: 5321) |
| ADE2_R | AAACAACAAAAGGATACTAGTCGCTATCCTCGGTTCTGCAT (SEQ ID |
| | NO: 5322) |
| ADE2_TGA_R | TTAGTAAATGGTGCTCATTTTTCGGCGTACA (SEQ ID NO: 5323) |
| ADE2_TGA_F | TGTACGCCGAAAAATGAGCACCATTTACTAA (SEQ ID NO: 5324) |
| LEU2_F | AACTGTGGGAATACTCAGGTATCGT (SEQ ID NO: 5325) |
| LEU2_R | TTAAGCAAGGATTTTCTTAACTTCTTCGGC (SEQ ID NO: 5326) |
| ADAR2_yeast_F | CCAGATCGGGGGTTCCGGCGGGTCC (SEQ ID NO: 5327) |
| ADAR2_yeast_R | GAACAAAAGCTGGAGCTCCACCG (SEQ ID NO: 5328) |
| E620G_yeast_R | GACGCCCTGCCTAACcCATCTTTGCCGGTCG (SEQ ID NO: 5329) |
| E620G_yeast_F | CGACCGGCAAAGATGgGTTAGGCAGGGCGTC (SEQ ID NO: 5330) |
| ADE2 targeting spacer | CGTCAATGGTGCcCATTTTTCGGCGTACAAAGGA (SEQ ID NO: 5331) |
| (22 bp mismatch) | |

TABLE 21
Primers for REPAIR Round 1, 2 screen mutant cloning into mammalian
Table 21-A

| Well | Name | Sequence |
|---|---|---|
| A01 | A09D_piece1R | CAGGCGTGAGACAGCGTCATCTAAAACCTGCGGTAA (SEQ ID NO: |
| | | 5332) |
| A02 | A11T_piece1R | CAGGACCAGGCGTGAGACCGTGTCAGCTAAAACCTG (SEQ ID NO: |
| | | 5333) |
| A03 | L17P_piece1R | CAGGTCACCAAACTTACCGGGGACCAGGCGTGAGAC (SEQ ID NO: |
| | | 5334) |
| A04 | G21D_piece1R | GAAGTTGTCGGTCAGGTCGTCAAACTTACCCAGGAC (SEQ ID NO: 5335) |
| A05 | T24A_piece1R | AGGGGAGGAGAAGTTGTCTGCCAGGTCACCAAACTT (SEQ ID NO: |
| | | 5336) |
| A06 | T24S_piece1R | AGGGGAGGAGAAGTTGTCGGACAGGTCACCAAACTT (SEQ ID NO: |
| | | 5337) |
| A07 | N26I_piece1R | AGCGTGAGGGGAGGAGAATATGTCGGTCAGGTCACC (SEQ ID NO: |
| | | 5338) |
| A08 | F27S_piece1R | GCGAGCGTGAGGGGAGGAGGAGTTGTCGGTCAGGTC (SEQ ID NO: |
| | | 5339) |
| A09 | R33G_piece1R | TCCAGCCAGCACTTTTCTGCCAGCGTGAGGGGAGGA (SEQ ID NO: 5340) |
| A10 | K35R_piece1R | GACGACTCCAGCCAGCACTCTTCTGCGAGCGTGAGG (SEQ ID NO: 5341) |
| A11 | L37V_piece1R | TGTCATGACGACTCCAGCTACCACTTTTCTGCGAGC (SEQ ID NO: 5342) |
| A12 | V40G_piece1R | TGTGCCTGTTGTCATGACGCCTCCAGCCAGCACTTT (SEQ ID NO: 5343) |
| B01 | T44S_piece1R | ATCTTTAACATCTGTGCCAGATGTCATGACGACTCC (SEQ ID NO: 5344) |
| B02 | K49R_piece1R | ACTTATCACCTTGGCATCTCTAACATCTGTGCCTGT (SEQ ID NO: 5345) |
| B03 | I54L_piece1R | TGTTCCTGTAGAAACACTGAGCACCTTGGCATCTTT (SEQ ID NO: 5346) |
| B04 | V56A_piece1R | ACATTTTGTTCCTGTAGATGCACTTATCACCTTGGC (SEQ ID NO: 5347) |
| B05 | V56E_piece1R | ACATTTTGTTCCTGTAGACTCACTTATCACCTTGGC (SEQ ID NO: 5348) |
| B06 | G59R_piece1R | ACCATTAATACATTTTGTCCGTGTAGAAACACTTAT (SEQ ID NO: 5349) |
| B07 | T60A_piece1R | TTCACCATTAATACATTTAGCTCCTGTAGAAACACT (SEQ ID NO: 5350) |
| B08 | T60S_piece1R | TTCACCATTAATACATTTACTTCCTGTAGAAACACT (SEQ ID NO: 5351) |
| B09 | C62F_piece1R | CATGTATTCACCATTAATAAATTTTGTTCCTGTAGA (SEQ ID NO: 5352) |
| B10 | G65D_piece1R | ACGATCACTCATGTATTCGTCATTAATACATTTTGT (SEQ ID NO: 5353) |
| B11 | L75Q_piece1R | TTCTGCATGGCAGTCATTCTGTGCAAGGCCACGATC (SEQ ID NO: 5354) |
| B12 | C78R_piece1R | AGATATTATTTCTGCATGACGGTCATTTAATGCAAG (SEQ ID NO: 5355) |
| C01 | I83T_piece1R | GAGCAAGGATCTCCGAGAGGTTATTTCTGCATGGCA (SEQ ID NO: 5356) |
| C02 | I83N_piece1R | GAGCAAGGATCTCCGAGAATTTATTTCTGCATGGCA (SEQ ID NO: 5357) |
| C03 | R86G_piece1R | AAGAAATCTGAGCAAGGAGCCCCGAGATATTATTTC (SEQ ID NO: |
| | | 5358) |
| C04 | S87N_piece1R | ATAAAGAAATCTGAGCAAATTTCTCCGAGATATTAT (SEQ ID NO: 5359) |
| C05 | L92M_piece1R | AAGCTCAAGTTGTGTATACATAAATCTGAGCAAGGA (SEQ ID NO: |
| | | 5360) |
| C06 | K103R_piece1R | GGATCTTTTTTGATCATCCCGGTTATTTAAGTAAAG (SEQ ID NO: 5361) |
| C07 | D104G_piece1R | GATGGATCTTTTTTGATCGCCTTTGTTATTTAAGTA (SEQ ID NO: 5362) |
| C08 | D105V_piece1R | AAAGATGGATCTTTTTTGCACATCTTTGTTATTTAA (SEQ ID NO: 5363) |
| C09 | D105Y_piece1R | AAAGATGGATCTTTTTTGATAATCTTTGTTATTTAA (SEQ ID NO: 5364) |
| C10 | S109G_piece1R | CTCTGATTTCTGAAAGATGCCTCTTTTTTGATCATC (SEQ ID NO: 5365) |
| C11 | F111L_piece1R | CCCTCGCTCTGATTTCTGCAAGATGGATCTTTTTTG (SEQ ID NO: 5366) |
| C12 | Q112L_piece1R | CCCCCCTCGCTCTGATTTCAGAAAGATGGATCTTTT (SEQ ID NO: 5367) |
| D01 | R116C_piece1R | CTTCAGCCTAAACCCCCCACACTCTGATTTCTGAAA (SEQ ID NO: 5368) |
| D02 | G117R_piece1R | CTCCTTCAGCCTAAACCCTCTTCGCTCTGATTTCTG (SEQ ID NO: 5369) |
| D03 | R120I_piece1R | CTGGACATTCTCCTTCAGGATAAACCCCCCTCGCTC (SEQ ID NO: 5370) |
| D04 | E123V_piece1R | CAGATGAAACTGGACATTAACCTTCAGCCTAAACCC (SEQ ID NO: 5371) |
| D05 | N124K_piece1R | GTACAGATGAAACTGGACCTTCTCCTTCAGCCTAAA (SEQ ID NO: 5372) |
| D06 | L129P_piece1R | GGGAGAGGTGCTGATGTATGGATGAAACTGGACATT (SEQ ID NO: |
| | | 5373) |
| D07 | Y130N_piece1R | ACAGGGAGAGGTGCTGATGTTCAGATGAAACTGGAC (SEQ ID NO: |
| | | 5374) |
| D08 | S134P_piece1R | TCTGGCATCTCCACAGGGAGGGGTGCTGATGTACAG (SEQ ID NO: 5375) |
| D09 | C136R_piece1R | GAAGATTCTGGCATCTCCTCTGGGAGAGGTGCTGAT (SEQ ID NO: 5376) |
| D10 | A139T_piece1R | ATGTGGTGAGAAGATTCTTGTATCTCCACAGGGAGA (SEQ ID NO: 5377) |
| D11 | F142L_piece1R | GATTGGCTCATGTGGTGAGAGGATTCTGGCATCTCC (SEQ ID NO: 5378) |
| D12 | H145L_piece1R | TTCTTCCAGGATTGGCTCAAGTGGTGAGAAGATTCT (SEQ ID NO: 5379) |
| E01 | L149M_piece1R | TCTATCTGCTGGTTCTTCCATGATTGGCTCATGTGG (SEQ ID NO: 5380) |
| E02 | H156L_piece1R | TCTTGCTTTACGATTTGGCAGTCTATCTGCTGGTTC (SEQ ID NO: 5381) |
| E03 | P157L_piece1R | TCCTCTTGCTTTACGATTGAGGTGTCTATCTGCTGG (SEQ ID NO: 5382) |
| E04 | P157Q_piece1R | TCCTCTTGCTTTACGATTCTGGTGTCTATCTGCTGG (SEQ ID NO: 5383) |
| E05 | N158R_piece1R | CTGTCCTCTTGCTTTACGGCGTGGGTGTCTATCTGC (SEQ ID NO: 5384) |
| E06 | N158K_piece1R | CTGTCCTCTTGCTTTACGCTTTGGGTGTCTATCTGC (SEQ ID NO: 5385) |
| E07 | K160R_piece1R | CCGTAGCTGTCCTCTTGCCCGACGATTTGGGTGTCT (SEQ ID NO: 5386) |
| E08 | K160T_piece1R | CCGTAGCTGTCCTCTTGCCGTACGATTTGGGTGTCT (SEQ ID NO: 5387) |
| E09 | A161T_piece1R | GGTCCGTAGCTGTCCTCTTGTTTTACGATTTGGGTG (SEQ ID NO: 5388) |
| E10 | R162I_piece1R | TTTGGTCCGTAGCTGTCCTATTGCTTTACGATTTGG (SEQ ID NO: 5389) |
| E11 | R162S_piece1R | TTTGGTCCGTAGCTGTCCGCTTGCTTTACGATTTGG (SEQ ID NO: 5390) |
| E12 | Q164R_piece1R | CTCTATTTTGGTCCGTAGTCTTCCTCTTGCTTTACG (SEQ ID NO: 5391) |
| F01 | Q164E_piece1R | CTCTATTTTGGTCCGTAGTTCTCCTCTTGCTTTACG (SEQ ID NO: 5392) |
| F02 | Q164K_piece1R | CTCTATTTTGGTCCGTAGTTTTCCTCTTGCTTTACG (SEQ ID NO: 5393) |
| F03 | L165M_piece1R | AGACTCTATTTTGGTCCGCATCTGTCCTCTTGCTTT (SEQ ID NO: 5394) |
| F04 | I169T_piece1R | CGTCCCCTGACCAGACTCCGTTTTGGTCCGTAGCTG (SEQ ID NO: 5395) |
| F05 | E170G_piece1R | AATCGTCCCCTGACCAGACCCTATTTTGGTCCGTAG (SEQ ID NO: 5396) |
| F06 | Q173R_piece1R | GCGCACTGGAATCGTCCCGCGACCAGACTCTATTTT (SEQ ID NO: 5397) |
| F07 | I176M_piece1R | CGCATTGGAGCGCACTGGCATCGTCCCCTGACCAGA (SEQ ID NO: 5398) |
| F08 | V178L_piece1R | GATGCTCGCATTGGAGCGGAGTGGAATCGTCCCCTG (SEQ ID NO: 5399) |
| F09 | N181K_piece1R | CCACGTTTGGATGCTCGCTTTGGAGCGCACTGGAAT (SEQ ID NO: 5400) |
| F10 | S183G_piece1R | CCCGTCCCACGTTTGGATGCCCGCATTGGAGCGCAC (SEQ ID NO: 5401) |
| F11 | I184L_piece1R | CACCCCGTCCCACGTTTGAAGGCTCGCATTGGAGCG (SEQ ID NO: 5402) |
| F12 | Q185L_piece1R | CAGCACCCCGTCCCACGTCAAGATGCTCGCATTGGA (SEQ ID NO: |
| | | 5403) |
| G01 | Q185R_piece1R | CAGCACCCCGTCCCACGTCCGGATGCTCGCATTGGA (SEQ ID NO: 5404) |
| G02 | G189A_piece1R | CCGCTCCCCTTGCAGCACAGCGTCCCACGTTTGGAT (SEQ ID NO: 5405) |
| G03 | M191V_piece1R | GAGCAGCCGCTCCCCTTGCACCACCCCGTCCCACGT (SEQ ID NO: 5406) |
| G04 | R195I_piece1R | GCAGGACATGGTGAGCAGGATCTCCCCTTGCAGCAC (SEQ ID NO: |
| | | 5407) |
| G05 | L196Q_piece1R | ACTGCAGGACATGGTGAGCTGCCGCTCCCCTTGCAG (SEQ ID NO: 5408) |
| G06 | S200N_piece1R | TGCAATCTTGTCACTGCAGTTCATGGTGAGCAGCCG (SEQ ID NO: 5409) |
| G07 | S202P_piece1R | CCAGCGTGCAATCTTGTCGGGGCAGGACATGGTGAG (SEQ ID NO: |
| | | 5410) |
| G08 | I205T_piece1R | CACCACGTTCCAGCGTGCGGTCTTGTCACTGCAGGA (SEQ ID NO: 5411) |
| G09 | I205V_piece1R | CACCACGTTCCAGCGTGCCACCTTGTCACTGCAGGA (SEQ ID NO: 5412) |
| G10 | A206P_piece1R | GCCCACCACGTTCCAGCGAGGAATCTTGTCACTGCA (SEQ ID NO: 5413) |
| G11 | R207S_piece1R | GATGCCCACCACGTTCCAGCTTGCAATCTTGTCACT (SEQ ID NO: 5414) |
| G12 | N209I_piece1R | TCCCTGGATGCCCACCACTATCCAGCGTGCAATCTT (SEQ ID NO: 5415) |
| H01 | N209K_piece1R | TCCCTGGATGCCCACCACCTTCCAGCGTGCAATCTT (SEQ ID NO: 5416) |
| H02 | V211E_piece1R | CAGTGATCCCTGGATGCCCTCCACGTTCCAGCGTGC (SEQ ID NO: 5417) |
| H03 | G212S_piece1R | GAGCAGTGATCCCTGGATACTCACCACGTTCCAGCG (SEQ ID NO: 5418) |
| H04 | L218F_piece1R | GGGCTCCACGAAAATGCTAAACAGTGATCCCTGGAT (SEQ ID NO: |
| | | 5419) |
| H05 | V222I_piece1R | CGAGAAGTAAATGGGCTCGATGAAAATGCTGAGCAG (SEQ ID NO: |
| | | 5420) |
| H06 | I225N_piece1R | GATGATGCTCGAGAAGTAGTTGGGCTCCACGAAAAT (SEQ ID NO: |
| | | 5421) |
| H07 | Y226H_piece1R | CAGGATGATGCTCGAGAAGTGAATGGGCTCCACGAA (SEQ ID NO: |
| | | 5422) |
| H08 | S228R_piece1R | GCTGCCCAGGATGATGCTTCTGAAGTAAATGGGCTC (SEQ ID NO: 5423) |
| H09 | I230V_piece1R | GTAAAGGCTGCCCAGGATCACGCTCGAGAAGTAAAT (SEQ ID NO: |
| | | 5424) |
| H10 | I231M_piece1R | GTGGTAAAGGCTGCCCAGCATGATGCTCGAGAAGTA (SEQ ID NO: |
| | | 5425) |
| H11 | S234N_piece1R | GTGGTCCCCGTGGTAAAGGTTGCCCAGGATGATGCT (SEQ ID NO: 5426) |
| H12 | G238V_piece1R | GGCCCTGGAAAGGTGGTCGACGTGGTAAAGGCTGCC (SEQ ID NO: |
| | | 5427) |

TABLE 21-B

| Well | Name | Sequence |
|---|---|---|
| A01 | A09D_piece2F | TTACCGCAGGTTTTAGATGACGCTGTCTCACGCCTG (SEQ ID NO: 5428) |
| A02 | AllT_piece2F | CAGGTTTTAGCTGACACGGTCTCACGCCTGGTCCTG (SEQ ID NO: 5429) |
| A03 | L17P_piece2F | GTCTCACGCCTGGTCCCCGGTAAGTTTGGTGACCTG (SEQ ID NO: 5430) |
| A04 | G21D_piece2F | GTCCTGGGTAAGTTTGACGACCTGACCGACAACTTC (SEQ ID NO: 5431) |
| A05 | T24A_piece2F | AAGTTTGGTGACCTGGCAGACAACTTCTCCTCCCCT (SEQ ID NO: 5432) |
| A06 | T24S_piece2F | AAGTTTGGTGACCTGTCCGACAACTTCTCCTCCCCT (SEQ ID NO: 5433) |
| A07 | N26I_piece2F | GGTGACCTGACCGACATATTCTCCTCCCCTCACGCT (SEQ ID NO: 5434) |
| A08 | F27S_piece2F | GACCTGACCGACAACTCCTCCTCCCCTCACGCTCGC (SEQ ID NO: 5435) |
| A09 | R33G_piece2F | TCCTCCCCTCACGCTGGCAGAAAAGTGCTGGCTGGA (SEQ ID NO: 5436) |
| A10 | K35R_piece2F | CCTCACGCTCGCAGAAGAGTGCTGGCTGGAGTCGTC (SEQ ID NO: 5437) |
| A11 | L37V_piece2F | GCTCGCAGAAAAGTGGTAGCTGGAGTCGTCATGACA (SEQ ID NO: |
| | | 5438) |
| A12 | V40G_piece2F | AAAGTGCTGGCTGGAGGCGTCATGACAACAGGCACA (SEQ ID NO: |
| | | 5439) |
| B01 | T44S_piece2F | GGAGTCGTCATGACATCTGGCACAGATGTTAAAGAT (SEQ ID NO: 5440) |
| B02 | K49R_piece2F | ACAGGCACAGATGTTAGAGATGCCAAGGTGATAAGT (SEQ ID NO: |
| | | 5441) |
| B03 | I54L_piece2F | AAAGATGCCAAGGTGCTCAGTGTTTCTACAGGAACA (SEQ ID NO: 5442) |
| B04 | V56A_piece2F | GCCAAGGTGATAAGTGCATCTACAGGAACAAAATGT (SEQ ID NO: |
| | | 5443) |
| B05 | V56E_piece2F | GCCAAGGTGATAAGTGAGTCTACAGGAACAAAATGT (SEQ ID NO: |
| | | 5444) |
| B06 | G59R_piece2F | ATAAGTGTTTCTACACGGACAAAATGTATTAATGGT (SEQ ID NO: 5445) |
| B07 | T60A_piece2F | AGTGTTTCTACAGGAGCTAAATGTATTAATGGTGAA (SEQ ID NO: 5446) |
| B08 | T60S_piece2F | AGTGTTTCTACAGGAAGTAAATGTATTAATGGTGAA (SEQ ID NO: 5447) |
| B09 | C62F_piece2F | TCTACAGGAACAAAATTTATTAATGGTGAATACATG (SEQ ID NO: 5448) |
| B10 | G65D_piece2F | ACAAAATGTATTAATGACGAATACATGAGTGATCGT (SEQ ID NO: 5449) |
| B11 | L75Q_piece2F | GATCGTGGCCTTGCACAGAATGACTGCCATGCAGAA (SEQ ID NO: 5450) |
| B12 | C78R_piece2F | CTTGCATTAAATGACCGTCATGCAGAAATAATATCT (SEQ ID NO: 5451) |
| C01 | I83T_piece2F | TGCCATGCAGAAATAACCTCTCGGAGATCCTTGCTC (SEQ ID NO: 5452) |
| C02 | I83N_piece2F | TGCCATGCAGAAATAAATTCTCGGAGATCCTTGCTC (SEQ ID NO: 5453) |
| C03 | R86G_piece2F | GAAATAATATCTCGGGGCTCCTTGCTCAGATTTCTT (SEQ ID NO: 5454) |
| C04 | S87N_piece2F | ATAATATCTCGGAGAAATTTGCTCAGATTTCTTTAT (SEQ ID NO: 5455) |
| C05 | L92M_piece2F | TCCTTGCTCAGATTTATGTATACACAACTTGAGCTT (SEQ ID NO: 5456) |
| C06 | K103R_piece2F | CTTTACTTAAATAACCGGGATGATCAAAAAAGATCC (SEQ ID NO: 5457) |
| C07 | D104G_piece2F | TACTTAAATAACAAAGGCGATCAAAAAAGATCCATC (SEQ ID NO: |
| | | 5458) |
| C08 | D105V_piece2F | TTAAATAACAAAGATGTGCAAAAAAGATCCATCTTT (SEQ ID NO: 5459) |
| C09 | D105Y_piece2F | TTAAATAACAAAGATTATCAAAAAAGATCCATCTTT (SEQ ID NO: 5460) |
| C10 | S109G_piece2F | GATGATCAAAAAAGAGGCATCTTTCAGAAATCAGAG (SEQ ID NO: |
| | | 5461) |
| C11 | F111L_piece2F | CAAAAAAGATCCATCTTGCAGAAATCAGAGCGAGGG (SEQ ID NO: |
| | | 5462) |
| C12 | Q112L_piece2F | AAAAGATCCATCTTTCTGAAATCAGAGCGAGGGGGG (SEQ ID NO: |
| | | 5463) |
| D01 | R116C_piece2F | TTTCAGAAATCAGAGTGTGGGGGGTTTAGGCTGAAG (SEQ ID NO: 5464) |
| D02 | G117R_piece2F | CAGAAATCAGAGCGAAGAGGGTTTAGGCTGAAGGAG (SEQ ID NO: |
| | | 5465) |
| D03 | R120I_piece2F | GAGCGAGGGGGGTTTATCCTGAAGGAGAATGTCCAG (SEQ ID NO: |
| | | 5466) |
| D04 | E123V_piece2F | GGGTTTAGGCTGAAGGTTAATGTCCAGTTTCATCTG (SEQ ID NO: 5467) |
| D05 | N124K_piece2F | TTTAGGCTGAAGGAGAAGGTCCAGTTTCATCTGTAC (SEQ ID NO: 5468) |
| D06 | L129P_piece2F | AATGTCCAGTTTCATCCATACATCAGCACCTCTCCC (SEQ ID NO: 5469) |
| D07 | Y130N_piece2F | GTCCAGTTTCATCTGAACATCAGCACCTCTCCCTGT (SEQ ID NO: 5470) |
| D08 | S134P_piece2F | CTGTACATCAGCACCCCTCCCTGTGGAGATGCCAGA (SEQ ID NO: 5471) |
| D09 | C136R_piece2F | ATCAGCACCTCTCCCAGAGGAGATGCCAGAATCTTC (SEQ ID NO: 5472) |
| D10 | A139T_piece2F | TCTCCCTGTGGAGATACAAGAATCTTCTCACCACAT (SEQ ID NO: 5473) |
| D11 | F142L_piece2F | GGAGATGCCAGAATCCTCTCACCACATGAGCCAATC (SEQ ID NO: 5474) |
| D12 | H145L_piece2F | AGAATCTTCTCACCACTTGAGCCAATCCTGGAAGAA (SEQ ID NO: 5475) |
| E01 | L149M_piece2F | CCACATGAGCCAATCATGGAAGAACCAGCAGATAGA (SEQ ID NO: |
| | | 5476) |
| E02 | H156L_piece2F | GAACCAGCAGATAGACTGCCAAATCGTAAAGCAAGA (SEQ ID NO: |
| | | 5477) |
| E03 | P157L_piece2F | CCAGCAGATAGACACCTCAATCGTAAAGCAAGAGGA (SEQ ID NO: |
| | | 5478) |
| E04 | P157Q_piece2F | CCAGCAGATAGACACCAGAATCGTAAAGCAAGAGGA (SEQ ID NO: |
| | | 5479) |
| E05 | N158R_piece2F | GCAGATAGACACCCACGCCGTAAAGCAAGAGGACAG (SEQ ID NO: |
| | | 5480) |
| E06 | N158K_piece2F | GCAGATAGACACCCAAAGCGTAAAGCAAGAGGACAG (SEQ ID NO: |
| | | 5481) |
| E07 | K160R_piece2F | AGACACCCAAATCGTCGGGCAAGAGGACAGCTACGG (SEQ ID NO: |
| | | 5482) |
| E08 | K160T_piece2F | AGACACCCAAATCGTACGGCAAGAGGACAGCTACGG (SEQ ID NO: |
| | | 5483) |
| E09 | A161T_piece2F | CACCCAAATCGTAAAACAAGAGGACAGCTACGGACC (SEQ ID NO: |
| | | 5484) |
| E10 | R162I_piece2F | CCAAATCGTAAAGCAATAGGACAGCTACGGACCAAA (SEQ ID NO: |
| | | 5485) |
| E11 | R162S_piece2F | CCAAATCGTAAAGCAAGCGGACAGCTACGGACCAAA (SEQ ID NO: |
| | | 5486) |
| E12 | Q164R_piece2F | CGTAAAGCAAGAGGAAGACTACGGACCAAAATAGAG (SEQ ID NO: |
| | | 5487) |
| F01 | Q164E_piece2F | CGTAAAGCAAGAGGAGAACTACGGACCAAAATAGAG (SEQ ID NO: |
| | | 5488) |
| F02 | Q164K_piece2F | CGTAAAGCAAGAGGAAAACTACGGACCAAAATAGAG (SEQ ID NO: |
| | | 5489) |
| F03 | L165M_piece2F | AAAGCAAGAGGACAGATGCGGACCAAAATAGAGTCT (SEQ ID NO: |
| | | 5490) |
| F04 | I169T_piece2F | CAGCTACGGACCAAAACGGAGTCTGGTCAGGGGACG (SEQ ID NO: |
| | | 5491) |
| F05 | E170G_piece2F | CTACGGACCAAAATAGGGTCTGGTCAGGGGACGATT (SEQ ID NO: |
| | | 5492) |
| F06 | Q173R_piece2F | AAAATAGAGTCTGGTCGCGGGACGATTCCAGTGCGC (SEQ ID NO: 5493) |
| F07 | 1176M_piece2F | TCTGGTCAGGGGACGATGCCAGTGCGCTCCAATGCG (SEQ ID NO: 5494) |
| F08 | V178L_piece2F | CAGGGGACGATTCCACTCCGCTCCAATGCGAGCATC (SEQ ID NO: 5495) |
| F09 | N181K_piece2F | ATTCCAGTGCGCTCCAAAGCGAGCATCCAAACGTGG (SEQ ID NO: 5496) |
| F10 | S183G_piece2F | GTGCGCTCCAATGCGGGCATCCAAACGTGGGACGGG (SEQ ID NO: |
| | | 5497) |
| F11 | I184L_piece2F | CGCTCCAATGCGAGCCTTCAAACGTGGGACGGGGTG (SEQ ID NO: 5498) |
| F12 | Q185L_piece2F | TCCAATGCGAGCATCTTGACGTGGGACGGGGTGCTG (SEQ ID NO: 5499) |
| G01 | Q185R_piece2F | TCCAATGCGAGCATCCGGACGTGGGACGGGGTGCTG (SEQ ID NO: |
| | | 5500) |
| G02 | G189A_piece2F | ATCCAAACGTGGGACGCTGTGCTGCAAGGGGAGCGG (SEQ ID NO: |
| | | 5501) |
| G03 | M191V_piece2F | ACGTGGGACGGGGTGGTGCAAGGGGAGCGGCTGCTC (SEQ ID NO: |
| | | 5502) |
| G04 | R195I_piece2F | GTGCTGCAAGGGGAGATCCTGCTCACCATGTCCTGC (SEQ ID NO: 5503) |
| G05 | L196Q_piece2F | CTGCAAGGGGAGCGGCAGCTCACCATGTCCTGCAGT (SEQ ID NO: 5504) |
| G06 | S200N_piece2F | CGGCTGCTCACCATGAACTGCAGTGACAAGATTGCA (SEQ ID NO: 5505) |
| G07 | S202P_piece2F | CTCACCATGTCCTGCCCCGACAAGATTGCACGCTGG (SEQ ID NO: 5506) |
| G08 | I205T_piece2F | TCCTGCAGTGACAAGACCGCACGCTGGAACGTGGTG (SEQ ID NO: 5507) |
| G09 | I205V_piece2F | TCCTGCAGTGACAAGGTGGCACGCTGGAACGTGGTG (SEQ ID NO: 5508) |
| G10 | A206P_piece2F | TGCAGTGACAAGATTCCTCGCTGGAACGTGGTGGGC (SEQ ID NO: 5509) |
| G11 | R207S_piece2F | AGTGACAAGATTGCAAGCTGGAACGTGGTGGGCATC (SEQ ID NO: |
| | | 5510) |
| G12 | N209I_piece2F | AAGATTGCACGCTGGATAGTGGTGGGCATCCAGGGA (SEQ ID NO: |
| | | 5511) |
| H01 | N209K_piece2F | AAGATTGCACGCTGGAAGGTGGTGGGCATCCAGGGA (SEQ ID NO: |
| | | 5512) |
| H02 | V211E_piece2F | GCACGCTGGAACGTGGAGGGCATCCAGGGATCACTG (SEQ ID NO: |
| | | 5513) |
| H03 | G212S_piece2F | CGCTGGAACGTGGTGAGTATCCAGGGATCACTGCTC (SEQ ID NO: 5514) |
| H04 | L218F_piece2F | ATCCAGGGATCACTGTTTAGCATTTTCGTGGAGCCC (SEQ ID NO: 5515) |
| H05 | V222I_piece2F | CTGCTCAGCATTTTCATCGAGCCCATTTACTTCTCG (SEQ ID NO: 5516) |
| H06 | I225N_piece2F | ATTTTCGTGGAGCCCAACTACTTCTCGAGCATCATC (SEQ ID NO: 5517) |
| H07 | Y226H_piece2F | TTCGTGGAGCCCATTCACTTCTCGAGCATCATCCTG (SEQ ID NO: 5518) |
| H08 | S228R_piece2F | GAGCCCATTTACTTCAGAAGCATCATCCTGGGCAGC (SEQ ID NO: 5519) |
| H09 | I230V_piece2F | ATTTACTTCTCGAGCGTGATCCTGGGCAGCCTTTAC (SEQ ID NO: 5520) |
| H10 | I231M_piece2F | TACTTCTCGAGCATCATGCTGGGCAGCCTTTACCAC (SEQ ID NO: 5521) |
| H11 | S234N_piece2F | AGCATCATCCTGGGCAACCTTTACCACGGGGACCAC (SEQ ID NO: 5522) |
| H12 | G238V_piece2F | GGCAGCCTTTACCACGTCGACCACCTTTCCAGGGCC (SEQ ID NO: 5523) |

TABLE 21-C

| Well | Name | Sequence |
|---|---|---|
| A01 | D239E_piece1R | CATGGCCCTGGAAAGGTGTTCCCCGTGGTAAAGGCT (SEQ ID NO: 5524) |
| A02 | R243H_piece1R | GATCCGCTGGTACATGGCGTGGGAAAGGTGGTCCCC (SEQ ID NO: 5525) |
| A03 | R243L_piece1R | GATCCGCTGGTACATGGCGAGGGAAAGGTGGTCCCC (SEQ ID NO: |
| | | 5526) |
| A04 | R243G_piece1R | GATCCGCTGGTACATGGCCCCGGAAAGGTGGTCCCC (SEQ ID NO: 5527) |
| A05 | Y246H_piece1R | TATGTTGGAGATCCGCTGGTGCATGGCCCTGGAAAG (SEQ ID NO: 5528) |
| A06 | Q247H_piece1R | CTCTATGTTGGAGATCCGATGGTACATGGCCCTGGA (SEQ ID NO: 5529) |
| A07 | R248S_piece1R | GTCCTCTATGTTGGAGATAGACTGGTACATGGCCCT (SEQ ID NO: 5530) |
| A08 | N251Y_piece1R | AGGTGGCAGGTCCTCTATGTAGGAGATCCGCTGGTA (SEQ ID NO: 5531) |
| A09 | S267F_piece1R | TTCTGCATTGCTGATGCCGAAGAGCAAAGGCTTGTT (SEQ ID NO: 5532) |
| A10 | S270P_piece1R | CTGCCGTGCTTCTGCATTTGGGATGCCACTGAGCAA (SEQ ID NO: 5533) |
| A11 | N282Y_piece1R | CGTCCAGTTGACACTGAAGTAGGGGGCCTTCCCTGG (SEQ ID NO: 5534) |
| A12 | V285G_piece1R | GTCGCCTACCGTCCAGTTGCCACTGAAGTTGGGGGC (SEQ ID NO: 5535) |
| B01 | A293V_piece1R | GGCGTTGATGACCTCAATTACGGAGTCGCCTACCGT (SEQ ID NO: 5536) |
| B02 | I294F_piece1R | CGTGGCGTTGATGACCTCAAAAGCGGAGTCGCCTAC (SEQ ID NO: 5537) |
| B03 | N298I_piece1R | ATCCTTCCCAGTCGTGGCGATGATGACCTCAATAGC (SEQ ID NO: 5538) |
| B04 | T301S_piece1R | GCCCAGCTCATCCTTCCCGCTCGTGGCGTTGATGAC (SEQ ID NO: 5539) |
| B05 | K303N_piece1R | CGCGCGGCCCAGCTCATCATTCCCAGTCGTGGCGTT (SEQ ID NO: 5540) |
| B06 | K303I_piece1R | CGCGCGGCCCAGCTCATCTATCCCAGTCGTGGCGTT (SEQ ID NO: 5541) |
| B07 | K303R_piece1R | CGCGCGGCCCAGCTCATCGCGCCCAGTCGTGGCGTT (SEQ ID NO: 5542) |
| B08 | E305G_piece1R | GCGGGACGCGCGGCCCAGGCCATCCTTCCCAGTCGT (SEQ ID NO: 5543) |
| B09 | R308S_piece1R | CTTACACAGGCGGGACGCAGAGCCCAGCTCATCCTT (SEQ ID NO: 5544) |
| B10 | K314E_piece1R | GCGACAGTACAACGCGTGTTCACACAGGCGGGACGC (SEQ ID NO: |
| | | 5545) |
| B11 | V328E_piece1R | GCGTAGTAAGTGGGAGGGCTCCTTGCCGTGCACACG (SEQ ID NO: 5546) |
| B12 | S330P_piece1R | CTTGGAGCGTAGTAAGTGGGGGGGAACCTTGCCGTG (SEQ ID NO: |
| | | 5547) |
| C01 | I337T_piece1R | GTACACGTTGGGCTTGGTGGTCTTGGAGCGTAGTAA (SEQ ID NO: 5548) |
| C02 | T338I_piece1R | ATGGTACACGTTGGGCTTAATAATCTTGGAGCGTAG (SEQ ID NO: 5549) |
| C03 | T338E_piece1R | ATGGTACACGTTGGGCTTCTCAATCTTGGAGCGTAG (SEQ ID NO: 5550) |
| C04 | H344R_piece1R | TGCCGCCAGCTTGGACTCCCTGTACACGTTGGGCTT (SEQ ID NO: 5551) |
| C05 | A350T_piece1R | GGCGGCCTGGTACTCCTTGGTCGCCAGCTTGGACTC (SEQ ID NO: 5552) |
| C06 | E352K_piece1R | CGCCTTGGCGGCCTGGTACTTCTTTGCCGCCAGCTT (SEQ ID NO: 5553) |
| C07 | A355V_piece1R | GAACAGACGCGCCTTGGCCACCTGGTACTCCTTTGC (SEQ ID NO: 5554) |
| C08 | R359G_piece1R | GATGAAGGCTGTGAACAGTCCCGCCTTGGCGGCCTG (SEQ ID NO: 5555) |
| C09 | R359M_piece1R | GATGAAGGCTGTGAACAGCATCGCCTTGGCGGCCTG (SEQ ID NO: 5556) |
| C10 | E378G_piece1R | GAGTGAGAACTGGTCCTGCCCGGTGGGCTTCTCCAC (SEQ ID NO: 5557) |
| C11 | Q381L_piece1R | TTACGTGAGTGAGAACAGGTCCTGCTCGGTGGG (SEQ ID NO: 5558) |
| C12 | | |
| D01 | | |
| D02 | | |
| D03 | | |
| D04 | | |
| D05 | | |
| D06 | | |
| D07 | | |
| D08 | | |
| D09 | | |
| D10 | | |
| D11 | | |
| D12 | | |
| E01 | D239E_piece2F | AGCCTTTACCACGGGGAACACCTTTCCAGGGCCATG (SEQ ID NO: 5559) |
| E02 | R243H_piece2F | GGGGACCACCTTTCCCACGCCATGTACCAGCGGATC (SEQ ID NO: 5560) |
| E03 | R243L_piece2F | GGGGACCACCTTTCCCTCGCCATGTACCAGCGGATC (SEQ ID NO: 5561) |
| E04 | R243G_piece2F | GGGGACCACCTTTCCGGGGCCATGTACCAGCGGATC (SEQ ID NO: 5562) |
| E05 | Y246H_piece2F | CTTTCCAGGGCCATGCACCAGCGGATCTCCAACATA (SEQ ID NO: 5563) |
| E06 | Q247H_piece2F | TCCAGGGCCATGTACCATCGGATCTCCAACATAGAG (SEQ ID NO: 5564) |
| E07 | R248S_piece2F | AGGGCCATGTACCAGTCTATCTCCAACATAGAGGAC (SEQ ID NO: 5565) |
| E08 | N251Y_piece2F | TACCAGCGGATCTCCTACATAGAGGACCTGCCACCT (SEQ ID NO: 5566) |
| E09 | S267F_piece2F | AACAAGCCTTTGCTCTTCGGCATCAGCAATGCAGAA (SEQ ID NO: 5567) |
| E10 | S270P_piece2F | TTGCTCAGTGGCATCCCAAATGCAGAAGCACGGCAG (SEQ ID NO: |
| | | 5568) |
| E11 | N282Y_piece2F | CCAGGGAAGGCCCCCTACTTCAGTGTCAACTGGACG (SEQ ID NO: 5569) |
| E12 | V285G_piece2F | GCCCCCAACTTCAGTGGCAACTGGACGGTAGGCGAC (SEQ ID NO: |
| | | 5570) |
| F01 | A293V_piece2F | ACGGTAGGCGACTCCGTAATTGAGGTCATCAACGCC (SEQ ID NO: 5571) |
| F02 | I294F_piece2F | GTAGGCGACTCCGCTTTTGAGGTCATCAACGCCACG (SEQ ID NO: 5572) |
| F03 | N298I_piece2F | GCTATTGAGGTCATCATCGCCACGACTGGGAAGGAT (SEQ ID NO: 5573) |
| F04 | T301S_piece2F | GTCATCAACGCCACGAGCGGGAAGGATGAGCTGGGC (SEQ ID NO: |
| | | 5574) |
| F05 | K303N_piece2F | AACGCCACGACTGGGAATGATGAGCTGGGCCGCGCG (SEQ ID NO: |
| | | 5575) |
| F06 | K303I_piece2F | AACGCCACGACTGGGATAGATGAGCTGGGCCGCGCG (SEQ ID NO: |
| | | 5576) |
| F07 | K303R_piece2F | AACGCCACGACTGGGCGCGATGAGCTGGGCCGCGCG (SEQ ID NO: |
| | | 5577) |
| F08 | E305G_piece2F | ACGACTGGGAAGGATGGCCTGGGCCGCGCGTCCCGC (SEQ ID NO: |
| | | 5578) |
| F09 | R308S_piece2F | AAGGATGAGCTGGGCTCTGCGTCCCGCCTGTGTAAG (SEQ ID NO: 5579) |
| F10 | K314E_piece2F | GCGTCCCGCCTGTGTGAACACGCGTTGTACTGTCGC (SEQ ID NO: 5580) |
| F11 | V328E_piece2F | CGTGTGCACGGCAAGGAGCCCTCCCACTTACTACGC (SEQ ID NO: 5581) |
| F12 | S330P_piece2F | CACGGCAAGGTTCCCCCCCACTTACTACGCTCCAAG (SEQ ID NO: 5582) |
| G01 | I337T_piece2F | TTACTACGCTCCAAGACCACCAAGCCCAACGTGTAC (SEQ ID NO: 5583) |
| G02 | T338I_piece2F | CTACGCTCCAAGATTATTAAGCCCAACGTGTACCAT (SEQ ID NO: 5584) |
| G03 | T338E_piece2F | CTACGCTCCAAGATTGAGAAGCCCAACGTGTACCAT (SEQ ID NO: 5585) |
| G04 | H344R_piece2F | AAGCCCAACGTGTACAGGGAGTCCAAGCTGGCGGCA (SEQ ID NO: |
| | | 5586) |
| G05 | A350T_piece2F | GAGTCCAAGCTGGCGACCAAGGAGTACCAGGCCGCC (SEQ ID NO: |
| | | 5587) |
| G06 | E352K_piece2F | AAGCTGGCGGCAAAGAAGTACCAGGCCGCCAAGGCG (SEQ ID NO: |
| | | 5588) |
| G07 | A355V_piece2F | GCAAAGGAGTACCAGGTGGCCAAGGCGCGTCTGTTC (SEQ ID NO: |
| | | 5589) |
| G08 | R359G_piece2F | CAGGCCGCCAAGGCGGGACTGTTCACAGCCTTCATC (SEQ ID NO: 5590) |
| G09 | R359M_piece2F | CAGGCCGCCAAGGCGATGCTGTTCACAGCCTTCATC (SEQ ID NO: 5591) |
| G10 | E378G_piece2F | GTGGAGAAGCCCACCGGGCAGGACCAGTTCTCACTC (SEQ ID NO: |
| | | 5592) |
| G11 | Q381L_piece2F | CCCACCGAGCAGGACCTGTTCTCACTCACGTAA (SEQ ID NO: 5593) |
| G12 | | |
| H01 | | |
| H02 | | |
| H03 | | |
| H04 | | |
| H05 | | |
| H06 | | |
| H07 | | |
| H08 | | |
| H09 | | |
| H10 | | |
| H11 | | |
| H12 | | |

TABLE 21-D

| Well | Name | Sequence |
|---|---|---|
| A01 | Q321H_piece1R | AGCGTCAGCTAAAACGTGCGGTAAATGCAGCTG (SEQ ID NO: 5594) |
| A02 | V322A_piece1R | GACAGCGTCAGCTAAGGCCTGCGGTAAATGCAG (SEQ ID NO: 5595) |
| A03 | V327I_piece1R | CAGGACCAGGCGTGAAATAGCGTCAGCTAAAAC (SEQ ID NO: 5596) |
| A04 | S328P_piece1R | ACCCAGGACCAGGCGTGGGACAGCGTCAGCTAA (SEQ ID NO: 5597) |
| A05 | T339M_piece1R | GGAGGAGAAGTTGTCCATCAGGTCACCAAACTT (SEQ ID NO: 5598) |
| A06 | P345L_pieceIR | TTTTCTGCGAGCGTGAAGGGAGGAGAAGTTGTC (SEQ ID NO: 5599) |
| A07 | V356G_piece1R | TGTGCCTGTTGTCATCCCGACTCCAGCCAGCAC (SEQ ID NO: 5600) |
| A08 | D365E_piece1R | ACTTATCACCTTGGCCTCTTTAACATCTGTGCC (SEQ ID NO: 5601) |
| A09 | I378F_piece1R | CATGTATTCACCATTGAAACATTTTGTTCCTGT (SEQ ID NO: 5602) |
| A10 | R386W_piece1R | ATTTAATGCAAGGCCCCAATCACTCATGTATTC (SEQ ID NO: 5603) |
| A11 | N391S_piece1R | TTCTGCATGGCAGTCGCTTAATGCAAGGCCACG (SEQ ID NO: 5604) |
| A12 | I397S_piece1R | GGATCTCCGAGATATGCTTTCTGCATGGCAGTC (SEQ ID NO: 5605) |
| B01 | L404M_piece1R | TGTATAAAGAAATCTCATCAAGGATCTCCGAGA (SEQ ID NO: 5606) |
| B02 | Q410R_piece1R | TAAGTAAAGCTCAAGGCGTGTATAAAGAAATCT (SEQ ID NO: 5607) |
| B03 | Y414H_piece1R | ATCTTTGTTATTTAAGTGAAGCTCAAGTTGTGT (SEQ ID NO: 5608) |
| B04 | Y414C_piece1R | ATCTTTGTTATTTAAGCAAAGCTCAAGTTGTGT (SEQ ID NO: 5609) |
| B05 | D419N_piece1R | GGATCTTTTTTGATCATTTTTGTTATTTAAGTA (SEQ ID NO: 5610) |
| B06 | R431H_piece1R | CAGCCTAAACCCCCCGTGCTCTGATTTCTGAAA (SEQ ID NO: 5611) |
| B07 | E438A_piece1R | ATGAAACTGGACATTAGCCTTCAGCCTAAACCC (SEQ ID NO: 5612) |
| B08 | F442L_piece1R | GCTGATGTACAGATGAAGCTGGACATTCTCCTT (SEQ ID NO: 5613) |
| B09 | T448I_piece1R | ATCTCCACAGGGAGAGATGCTGATGTACAGATG (SEQ ID NO: 5614) |
| B10 | F457I_piece1R | TGGCTCATGTGGTGAGATGATTCTGGCATCTCC (SEQ ID NO: 5615) |
| B11 | A468E_piece1R | ATTTGGGTGTCTATCTTCTGGTTCTTCCAGGAT (SEQ ID NO: 5616) |
| B12 | I484V_piece1R | CCCCTGACCAGACTCCACTTTGGTCCGTAGCTG (SEQ ID NO: 5617) |
| C01 | E485V_piece1R | CGTCCCCTGACCAGAGACTATTTTGGTCCGTAG (SEQ ID NO: 5618) |
| C02 | V505I_piece1R | CCGCTCCCCTTGCAGGATCCCGTCCCACGTTTG (SEQ ID NO: 5619) |
| C03 | E509V_piece1R | CATGGTGAGCAGCCGGACCCCTTGCAGCACCCC (SEQ ID NO: 5620) |
| C04 | C516F_piece1R | TGCAATCTTGTCACTGAAGGACATGGTGAGCAG (SEQ ID NO: 5621) |
| C05 | S517Y_piece1R | GCGTGCAATCTTGTCGTAGCAGGACATGGTGAG (SEQ ID NO: 5622) |
| C06 | L532I_piece1R | CACGAAAATGCTGAGGATTGATCCCTGGATGCC (SEQ ID NO: 5623) |
| C07 | I545N_piece1R | AAGGCTGCCCAGGATGTTGCTCGAGAAGTAAAT (SEQ ID NO: 5624) |
| C08 | S549T_piece1R | GTCCCCGTGGTAAAGAGTGCCCAGGATGATGCT (SEQ ID NO: 5625) |
| C09 | Y551N_piece1R | AAGGTGGTCCCCGTGGTTAAGGCTGCCCAGGAT (SEQ ID NO: 5626) |
| C10 | G553R_piece1R | CCTGGAAAGGTGGTCTCGGTGGTAAAGGCTGCC (SEQ ID NO: 5627) |
| C11 | G553E_piece1R | CCTGGAAAGGTGGTCCTCGTGGTAAAGGCTGCC (SEQ ID NO: 5628) |
| C12 | R563G_piece1R | CTCTATGTTGGAGATGCCCTGGTACATGGCCCT (SEQ ID NO: 5629) |
| D01 | I564F_piece1R | GTCCTCTATGTTGGAAAACCGCTGGTACATGGC (SEQ ID NO: 5630) |
| D02 | N566D_piece1R | TGGCAGGTCCTCTATGTCGGAGATCCGCTGGTA (SEQ ID NO: 5631) |
| D03 | A587V_piece1R | TGGCTGCCGTGCTTCCACATTGCTGATGCCACT (SEQ ID NO: 5632) |
| D04 | G593W_piece1R | GAAGTTGGGGGCCTTCCATGGCTGCCGTGCTTC (SEQ ID NO: 5633) |
| D05 | T603A_piece1R | AGCGGAGTCGCCTACTGCCCAGTTGACACTGAA (SEQ ID NO: 5634) |
| D06 | V611A_piece1R | AGTCGTGGCGTTGATAGCCTCAATAGCGGAGTC (SEQ ID NO: 5635) |
| D07 | A614G_piece1R | ATCCTTCCCAGTCGTCCCGTTGATGACCTCAAT (SEQ ID NO: 5636) |
| D08 | D619E_piece1R | CGCGCGGCCCAGGCCTTCCTTCCCAGTCGTGGC (SEQ ID NO: 5637) |
| D09 | R626K_piece1R | CGCGTGCTTACACAGCTTGGACGCGCGGCCCAG (SEQ ID NO: 5638) |
| D10 | C634W_piece1R | CACACGCATCCAGCGCCAGTACAACGCGTGCTT (SEQ ID NO: 5639) |
| D11 | I652V_piece1R | CACGTTGGGCTTGGTAACCTTGGAGCGTAGTAA (SEQ ID NO: 5640) |
| D12 | Q696H_piece1R | TTACGTGAGTGAGAAATGGTCCTGCTCGGTGGG (SEQ ID NO: 5641) |
| E01 | Q321H_piece2F | CAGCTGCATTTACCGCACGTTTTAGCTGACGCT (SEQ ID NO: 5642) |
| E02 | V322A_piece2F | CTGCATTTACCGCAGGCCTTAGCTGACGCTGTC (SEQ ID NO: 5643) |
| E03 | V327I_piece2F | GTTTTAGCTGACGCTATTTCACGCCTGGTCCTG (SEQ ID NO: 5644) |
| E04 | S328P_piece2F | TTAGCTGACGCTGTCCCACGCCTGGTCCTGGGT (SEQ ID NO: 5645) |
| E05 | T339M_piece2F | AAGTTTGGTGACCTGATGGACAACTTCTCCTCC (SEQ ID NO: 5646) |
| E06 | P345L_piece2F | GACAACTTCTCCTCCCTTCACGCTCGCAGAAAA (SEQ ID NO: 5647) |
| E07 | V356G_piece2F | GTGCTGGCTGGAGTCGGGATGACAACAGGCACA (SEQ ID NO: 5648) |
| E08 | D365E_piece2F | GGCACAGATGTTAAAGAGGCCAAGGTGATAAGT (SEQ ID NO: 5649) |
| E09 | I378F_piece2F | ACAGGAACAAAATGTTTCAATGGTGAATACATG (SEQ ID NO: 5650) |
| E10 | R386W_piece2F | GAATACATGAGTGATTGGGGCCTTGCATTAAAT (SEQ ID NO: 5651) |
| E11 | N391S_piece2F | CGTGGCCTTGCATTAAGCGACTGCCATGCAGAA (SEQ ID NO: 5652) |
| E12 | I397S_piece2F | GACTGCCATGCAGAAAGCATATCTCGGAGATCC (SEQ ID NO: 5653) |
| F01 | L404M_piece2F | TCTCGGAGATCCTTGATGAGATTTCTTTATACA (SEQ ID NO: 5654) |
| F02 | Q410R_piece2F | AGATTTCTTTATACACGCCTTGAGCTTTACTTA (SEQ ID NO: 5655) |
| F03 | Y414H_piece2F | ACACAACTTGAGCTTCACTTAAATAACAAAGAT (SEQ ID NO: 5656) |
| F04 | Y414C_piece2F | ACACAACTTGAGCTTTGCTTAAATAACAAAGAT (SEQ ID NO: 5657) |
| F05 | D419N_piece2F | TACTTAAATAACAAAAATGATCAAAAAAGATCC (SEQ ID NO: 5658) |
| F06 | R431H_piece2F | TTTCAGAAATCAGAGCACGGGGGGTTTAGGCTG (SEQ ID NO: 5659) |
| F07 | E438A_piece2F | GGGTTTAGGCTGAAGGCTAATGTCCAGTTTCAT (SEQ ID NO: 5660) |
| F08 | F442L_piece2F | AAGGAGAATGTCCAGCTTCATCTGTACATCAGC (SEQ ID NO: 5661) |
| F09 | T448I_piece2F | CATCTGTACATCAGCATCTCTCCCTGTGGAGAT (SEQ ID NO: 5662) |
| F10 | F457I_piece2F | GGAGATGCCAGAATCATCTCACCACATGAGCCA (SEQ ID NO: 5663) |
| F11 | A468E_piece2F | ATCCTGGAAGAACCAGAAGATAGACACCCAAAT (SEQ ID NO: 5664) |
| F12 | I484V_piece2F | CAGCTACGGACCAAAGTGGAGTCTGGTCAGGGG (SEQ ID NO: 5665) |
| G01 | E485V_piece2F | CTACGGACCAAAATAGTCTCTGGTCAGGGGACG (SEQ ID NO: 5666) |
| G02 | V505I_piece2F | CAAACGTGGGACGGGATCCTGCAAGGGGAGCGG (SEQ ID NO: 5667) |
| G03 | E509V_piece2F | GGGGTGCTGCAAGGGGTCCGGCTGCTCACCATG (SEQ ID NO: 5668) |
| G04 | C516F_piece2F | CTGCTCACCATGTCCTTCAGTGACAAGATTGCA (SEQ ID NO: 5669) |
| G05 | S517Y_piece2F | CTCACCATGTCCTGCTACGACAAGATTGCACGC (SEQ ID NO: 5670) |
| G06 | L532I_piece2F | GGCATCCAGGGATCAATCCTCAGCATTTTCGTG (SEQ ID NO: 5671) |
| G07 | I545N_piece2F | ATTTACTTCTCGAGCAACATCCTGGGCAGCCTT (SEQ ID NO: 5672) |
| G08 | S549T_piece2F | AGCATCATCCTGGGCACTCTTTACCACGGGGAC (SEQ ID NO: 5673) |
| G09 | Y551N_piece2F | ATCCTGGGCAGCCTTAACCACGGGGACCACCTT (SEQ ID NO: 5674) |
| G10 | G553R_piece2F | GGCAGCCTTTACCACCGAGACCACCTTTCCAGG (SEQ ID NO: 5675) |
| G11 | G553E_piece2F | GGCAGCCTTTACCACGAGGACCACCTTTCCAGG (SEQ ID NO: 5676) |
| G12 | R563G_piece2F | AGGGCCATGTACCAGGGCATCTCCAACATAGAG (SEQ ID NO: 5677) |
| H1 | I564F_piece2F | GCCATGTACCAGCGGTTTTCCAACATAGAGGAC (SEQ ID NO: 5678) |
| H2 | N566D_piece2F | TACCAGCGGATCTCCGACATAGAGGACCTGCCA (SEQ ID NO: 5679) |
| H3 | A587V_piece2F | AGTGGCATCAGCAATGTGGAAGCACGGCAGCCA (SEQ ID NO: 5680) |
| H4 | G593W_piece2F | GAAGCACGGCAGCCATGGAAGGCCCCCAACTTC (SEQ ID NO: 5681) |
| H5 | T603A_piece2F | TTCAGTGTCAACTGGGCAGTAGGCGACTCCGCT (SEQ ID NO: 5682) |
| H6 | V611A_piece2F | GACTCCGCTATTGAGGCTATCAACGCCACGACT (SEQ ID NO: 5683) |
| H7 | A614G_piece2F | ATTGAGGTCATCAACGGGACGACTGGGAAGGAT (SEQ ID NO: 5684) |
| H8 | D619E_piece2F | GCCACGACTGGGAAGGAAGGCCTGGGCCGCGCG (SEQ ID NO: 5685) |
| H9 | R626K_piece2F | CTGGGCCGCGCGTCCAAGCTGTGTAAGCACGCG (SEQ ID NO: 5686) |
| H10 | C634W_piece2F | AAGCACGCGTTGTACTGGCGCTGGATGCGTGTG (SEQ ID NO: 5687) |
| H11 | I652V_piece2F | TTACTACGCTCCAAGGTTACCAAGCCCAACGTG (SEQ ID NO: 5688) |
| H12 | Q696H_piece2F | CCCACCGAGCAGGACCATTTCTCACTCACGTAA (SEQ ID NO: 5689) |

TABLE 22
Next-generation sequencing library preparation first round PCR primers
for PFS screen

| Name | Sequence |
|---|---|
| PFS_NGS_F1 | CTTTCCCTACACGACGCTCTTCCGATCTCGCTAGCTCAGTCCTAGGTATAATGCTAGC |
| | (SEQ ID NO: 5690) |
| PFS_NGS_F1 | CTTTCCCTACACGACGCTCTTCCGATCTACGCTAGCTCAGTCCTAGGTATAATGCTAGC |
| | (SEQ ID NO: 5691) |
| PFS_NGS_F1 | CTTTCCCTACACGACGCTCTTCCGATCTGACGCTAGCTCAGTCCTAGGTATAATGCTAGC |
| | (SEQ ID NO: 5692) |
| PFS_NGS_F1 | CTTTCCCTACACGACGCTCTTCCGATCTTGACGCTAGCTCAGTCCTAGGTATAATGCTAGC |
| | (SEQ ID NO: 5693) |
| Cas13b- | GACTGGAGTTCAGACGTGTGCTCTTCCGATCTCCACAAATCGGAGGCTTCTTCAGC |
| t1_PFS_NGS_R | (SEQ ID NO: 5694) |
| Cas13b- | GACTGGAGTTCAGACGTGTGCTCTTCCGATCTCAAATCGGGGGCTTCTCCAGC |
| t2_PFS_NGS_R | (SEQ ID NO: 5695) |
| Cas13b- | GACTGGAGTTCAGACGTGTGCTCTTCCGATCTATCGGGGGCTGCTCCAGC |
| t3_PFS_NGS_R | (SEQ ID NO: 5696) |
| Cas13b- | GACTGGAGTTCAGACGTGTGCTCTTCCGATCTCCACAAATTGAGGCCCATCACAGC |
| t4_PFS_NGS_R | (SEQ ID NO: 5697) |
| Cas13b- | GACTGGAGTTCAGACGTGTGCTCTTCCGATCTCAAATCGAGGGCTGCTCCAGC |
| t5_PFS_NGS_R | (SEQ ID NO: 5698) |

TABLE 23
gRNA spacer sequences for Gaussia luciferase knockdown in
HEK293FT cells. Relative expression is as measured by depletion of luciferase activity
compared to a GFP control.

| | | Bt1 relative | Bt3 relative | Bt5 relative |
|---|---|---|---|---|
| | | expression | expression | expression |
| Name | Spacer sequence | (FIG. 1g) | (FIG. 1g) | (FIG. 1g) |
| Gaussia luciferase spacer | TTTGTCGCCTTCGTAGGTGTGGC | 0.55062748 | 0.38468809 | 0.3397597 |
| 1 | AGCGTCC (SEQ ID NO: 5699) | | | |
| Gaussia luciferase spacer | CCAGGAATCTCAGGAATGTCGAC | 0.5538685 | 0.35529333 | 0.44593268 |
| 2 | GATCGCC (SEQ ID NO: 5700) | | | |
| Gaussia luciferase spacer | GTCGACGATCGCCTCGCCTATGC | 0.38844716 | 0.29975324 | 0.45171626 |
| 3 | CGCCCTG (SEQ ID NO: 5701) | | | |
| Gaussia luciferase spacer | CGATGAACTGCTCCATGGGCTCC | 0.74248373 | 0.7076885 | 0.62729858 |
| 4 | AAGTCCT (SEQ ID NO: 5702) | | | |
| Gaussia luciferase spacer | TCGCGAAGTTGCTGGCCACGGCC | 0.48985892 | 0.56514571 | 0.34100497 |
| 5 | ACGATGT (SEQ ID NO: 5703) | | | |
| Gaussia luciferase spacer | CAGCCCCTGGTGCAGCCAGCTTT | 0.73143084 | 0.45223832 | 0.59533757 |
| 6 | CCGGGCA (SEQ ID NO: 5704) | | | |
| Gaussia luciferase spacer | GGCCCCCTTGATCTTGTCCACCT | 0.51439053 | 0.21018064 | 0.37180078 |
| 7 | GGCCCTG (SEQ ID NO: 5705) | | | |
| Gaussia luciferase spacer | GATGTGGGACAGGCAGATCAGA | 0.65183105 | 0.46064806 | 0.51302536 |
| 8 | CAGCCCCT (SEQ ID NO: 5706) | | | |
| Gaussia luciferase spacer | CGTTGCGGCAGCCACTTCTTGAG | 0.42079237 | 0.26868947 | 0.35726081 |
| 9 | CAGGTCA (SEQ ID NO: 5707) | | | |
| Gaussia luciferase spacer | TGTCGACGATCGCCTCGCCTATG | 0.63580411 | 0.30643568 | 0.36228356 |
| 10 | CCGCCCT (SEQ ID NO: 5708) | | | |
| Gaussia luciferase spacer | CTCGGCCACAGCGATGCAGATCA | 0.57120708 | 0.42967329 | 0.31493639 |
| 11 | GGGCAAA (SEQ ID NO: 5709) | | | |
| Gaussia luciferase spacer | CCTTGAACCCAGGAATCTCAGGA | 0.58010478 | 0.27618297 | 0.4351957 |
| 12 | ATGTCGA (SEQ ID NO: 5710) | | | |
| Gaussia luciferase spacer | CCGGGCATTGGCTTCCATCTCTT | 0.57770913 | 0.33286417 | 0.30132433 |
| 13 | TGAGCAC (SEQ ID NO: 5711) | | | |
| Gaussia luciferase spacer | ACAGGCAGATCAGACAGCCCCT | 0.77305496 | 0.54830739 | 0.69564972 |
| 14 | GGTGCAGC (SEQ ID NO: 5712) | | | |
| Gaussia luciferase spacer | GTCACCACCGGCCCCCTTGATCT | 0.74591779 | 0.65311879 | 0.65571849 |
| 15 | TGTCCAC (SEQ ID NO: 5713) | | | |
| Gaussia luciferase spacer | CTTGATGTGGGACAGGCAGATCA | 0.62758078 | 0.47392894 | 0.43519874 |
| 16 | GACAGCC (SEQ ID NO: 5714) | | | |
| Gaussia luciferase spacer | CTGGCCCTGGATCTTGCTGGCAA | 0.4753283 | 0.18260215 | 0.32822212 |
| 17 | AGGTCGC (SEQ ID NO: 5715) | | | |
| Gaussia luciferase spacer | GGGCTCCAAGTCCTTGAACCCAG | 0.60092434 | 0.3810874 | 0.42402921 |
| 18 | GAATCTC (SEQ ID NO: 5716) | | | |
| Gaussia luciferase spacer | ATGAACTGCTCCATGGGCTCCAA | 0.5442858 | 0.28338889 | 0.55534371 |
| 19 | GTCCTTG (SEQ ID NO: 5717) | | | |
| Gaussia luciferase spacer | TCGAGATCCGTGGTCGCGAAGTT | 0.48568996 | 0.35394464 | 0.36567816 |
| 20 | GCTGGCC (SEQ ID NO: 5718) | | | |
| Non-targeting spacer 1 | CCTCTGAAACGATGGTGCATGGT | 0.71872993 | 0.76013732 | 0.65806897 |
| | AGTGACC (SEQ ID NO: 5719) | | | |
| Non-targeting spacer 2 | CCTACAGGTTCTGAGTGGGTGCA | 0.85589957 | 0.70637393 | 0.70777278 |
| | CGGCCGT (SEQ ID NO: 5720) | | | |
| Non-targeting spacer 3 | GAAAATGGCCTATACCTTAGGGT | 0.78372124 | 0.72463671 | 0.71052405 |
| | TCGCGCG (SEQ ID NO: 5721) | | | |
| Non-targeting spacer 4 | GTAATGCCTGGCTTGTCGACGCA | 0.7999484 | 0.72841838 | 0.72973389 |
| | TAGTCTG (SEQ ID NO: 5722) | | | |
| Gaussia luciferase guide 1 | GGGCATTGGCTTCCATCTCTTTG | | | |
| (Supplementary FIG. 2) | AGCACCT (SEQ ID NO: 5723) | | | |
| Gaussia luciferase guide 2 | GGAATGTCGACGATCGCCTCGCC | | | |
| (Supplementary FIG. 2) | TATGCCG (SEQ ID NO: 5724) | | | |

TABLE 24
gRNA spacer sequences for endogenous transcript knockdown in
HEK293FT cells. Relative expression is as measured by qPCR as compared to GFP control.

| | | Bt1 relative | Bt3 relative |
|---|---|---|---|
| | | expression | expression |
| Name | Spacer sequence | (FIG. 1h) | (FIG. 1h) |
| CXCR4 spacer 1 | AGAGGTTGACTGTGTAGATGACATGGACTG | 0.4891226 | 0.43242082 |
| | (SEQ ID NO: 5725) | | |
| CXCR4 spacer 2 | GACAGGTGCAGCCTGTACTTGTCCGTCATG | 0.52526336 | 0.4274336 |
| | (SEQ ID NO: 5726) | | |
| CXCR4 spacer 3 | AAAGAGGAGGTCGGCCACTGACAGGTGCAG | 0.55379783 | 0.4922889 |
| | (SEQ ID NO: 5727) | | |
| STAT1 spacer 1 | CAGGAAGAAGGACAGATTCCTGGGTTCCGC | 0.1702036 | 0.26428803 |
| | (SEQ ID NO: 5728) | | |
| STAT1 spacer 2 | CCCAACATGTTCAGCTGGTCCACATTGAGA | 0.19275451 | 0.28221787 |
| | (SEQ ID NO: 5729) | | |
| STAT1 spacer 3 | ATTGAGACCTCTTTTGGTGACAGAAGAAAA | 0.32015 | 0.36930278 |
| | (SEQ ID NO: 5730) | | |
| STAT3 spacer 1 | GCAGCTCCTCAGTCACAATCAGGGAAGCAT | 0.54048912 | 0.43548581 |
| | (SEQ ID NO: 5731) | | |
| STAT3 spacer 2 | CGGTCTCAAAGGTGATCAGGTGCAGCTCCT | 0.61276978 | 0.49713932 |
| | (SEQ ID NO: 5732) | | |
| STAT3 spacer 3 | CTCGGTCTCAAAGGTGATCAGGTGCAGCTC | 0.5595753 | 0.4890901 |
| | (SEQ ID NO: 5733) | | |
| HRAS spacer 1 | GCGTGCAGCCAGGTCACACTTGTTCCCCAC | 0.43228711 | 0.40269921 |
| | (SEQ ID NO: 5734) | | |
| HRAS spacer 2 | GAGCCTGCCGAGATTCCACAGTGCGTGCAG | 0.84377946 | 0.35728849 |
| | (SEQ ID NO: 5735) | | |
| HRAS spacer 3 | AGTGCGTGCAGCCAGGTCACACTTGTTCCC | 0.49958394 | 0.47062756 |
| | (SEQ ID NO: 5736) | | |
| PPIB spacer 1 | CTGTCTTGGTGCTCTCCACCTTCCGCACCA | 0.47982775 | 0.42696786 |
| | (SEQ ID NO: 5737) | | |
| PPIB spacer 2 | GGGAGCCGTTGGTGTCTTTGCCTGCGTTGG | 0.33558372 | 0.33160707 |
| | (SEQ ID NO: 5738) | | |
| PPIB spacer 3 | CGTAGATGCTCTTTCCTCCTGTGCCATCTC | 0.36443656 | 0.3902864 |
| | (SEQ ID NO: 5739) | | |

TABLE 25
TaqMan probes used for qPCR

| Gene | TaqMan assay ID |
|---|---|
| CXCR4 | Hs00607978_s1 |
| STAT1 | Hs01013996_m1 |
| STAT3 | Hs00374280_m1 |
| HRAS | Hs00978050_g1 |
| PPIB | Hs00168719_m1 |
| GAPDH | Hs99999905_m1 |

TABLE 26
gRNA spacer sequences for Cypridina luciferase W85X reporter RNA
editing. Mismatch is denoted by lower case.

| | | | Cas13b-t1 | Cas13b-t3 |
|---|---|---|---|---|
| | Mismatch | | normalized RLU | normalized RLU |
| Site | distance | Spacer sequence | (FIG. 20B) | (FIG. 20B) |
| Cypridina | 2 | GAATCTCTTTCCATAGAATGTTCTAAACcA | 0.01260676 | 0.00488241 |
| luciferase | | (SEQ ID NO: 5740) | | |
| X95W | 4 | ATCTCTTTCCATAGAATGTTCTAAACcATC | 0.0085265 | 0.01001933 |
| | | (SEQ ID NO: 5741) | | |
| | 6 | CTCTTTCCATAGAATGTTCTAAACCATCCT | 0.01200875 | 0.00755633 |
| | | (SEQ ID NO: 5742) | | |
| | 8 | CTTTCCATAGAATGTTCTAAACCATCCTGC | 0,05522275 | 0.01003733 |
| | | (SEQ ID NO: 5743) | | |
| | 10 | TTCCATAGAATGTTCTAAACCATCCTGCGG | 0.079744 | 0.01706967 |
| | | (SEQ ID NO: 5744) | | |
| | 12 | CCATAGAATGTTCTAAACCATCCTGCGGCC | 0.085725 | 0.05851067 |
| | | (SEQ ID NO: 5745) | | |
| | 14 | ATAGAATGTTCTAAACCATCCTGCGGCCTC | 0.14881 | 0.05986233 |
| | | (SEQ ID NO: 5746) | | |
| | 16 | AGAATGTTCTAAACCATCCTGCGGCCTCTA | 0.1930325 | 0.06880567 |
| | | (SEQ ID NO: 5747) | | |
| | 18 | AATGTTCTAAACCATCCTGCGGCCTCTACT | 0.93053875 | 0.573424 |
| | | (SEQ ID NO: 5748) | | |
| | 20 | TGTTCTAAACcATCCTGCGGCCTCTACTCT | 0.99714725 | 0.69095267 |
| | | (SEQ ID NO: 5749) | | |
| | 22 | TTCTAAACCATCCTGCGGCCTCTACTCTGC | 1.028434 | 0.80134633 |
| | | (SEQ ID NO: 5750) | | |
| | 24 | CTAAACCATCCTGCGGCCTCTACTCTGCAT | 0.49195525 | 0.27232267 |
| | | (SEQ ID NO: 5751) | | |
| | 26 | AAACcATCCTGCGGCCTCTACTCTGCATTC | 0.35265576 | 0.36477816 |
| | | (SEQ ID NO: 5752) | | |
| | 28 | ACcATCCTGCGGCCTCTACTCTGCATTCAA | 0.59331175 | 0.36037733 |
| | | (SEQ ID NO: 5753) | | |
| | 30 | cATCCTGCGGCCTCTACTCTGCATTCAATT | 0.01181 | 0.00410067 |
| | | (SEQ ID NO: 5754) | | |
| Non- | | GTAATGCCTGGCTTGTCGACGCATAGTCTG | 0.007946 | 0.003682 |
| targeting | | (SEQ ID NO: 5755) | | |

Table 27
Optimal gRNA spacer sequences for RNA editing of endogenous
transcripts. Mismatch is denoted by lower case.

| | Mismatch | | Editing | Cas13b-t1 editing |
|---|---|---|---|---|
| Site | distance | Spacer sequence | system | rate (FIG. 20C) |
| STAT1 | 22 | TCTTGATAcATCCAGTTCCTTTAGGGCCAT | REPAIR | 0.2551795 |
| Y701C | | (SEQ ID NO: 5756) | | |
| STAT3 | 20 | GGTCTTCAGGCATGGGGCAGCGCTACCTGG | REPAIR | 0.21902363 |
| Y705C | | (SEQ ID NO: 5757) | | |
| LATS1 | 22 | TCGGAAGGCAAATTCATAGAATGCATGTTC | REPAIR | 0.20295815 |
| T1079A | | (SEQ ID NO: 5758) | | |
| CTNNB1 | 22 | AGCTGTGGCAGTGGCACCAGAATGGATTCC | REPAIR | 0.39486936 |
| T41A | | (SEQ ID NO: 5759) | | |
| Gaussia | 14 | TTCATCTTGGGCGTGCCATTGATGTGGGAC | RESCUE | 0.47878881 |
| luciferase | | (SEQ ID NO: 5760) | | |
| C82R | | | | |
| CTNNB1 | 22 | GAGCTGTGITAGTGGCACCAGAATGGATTC | RESCUE | 0.03803724 |
| T41I | | (SEQ ID NO: 5761) | | |
| KRAS D30D | 20 | GATCATATTCITCCACAAAATGATTCTGAA | RESCUE | 0.06281841 |
| | | (SEQ ID NO: 5762) | | |
| KRAS L56L | 22 | GCTGTGTCAGAATATCCAAGAGACAGGTT | RESCUE | 0.04002 |
| | | (SEQ ID NO: 5763) | | |

TABLE 28
Gene-specific reverse transcription primers

| Gene | RT primer sequence |
|---|---|
| Cypridina luciferase | TTTGCATTCATCTGGTACTTCTAGGGTGTC (SEQ ID NO: 5764) |
| STAT1 | TTCATCATACTGTCGAATTCTACAGAGCCC (SEQ ID NO: 5765) |
| CTNNB1 | TTACAGGTCAGTATCAAACCAGGCCAG (SEQ ID NO: 5766) |
| STAT3 | TTTCTGCAGCTTCCGTTCTCAGCTCCTCAC (SEQ ID NO: 5767) |
| LATS1 | TACTAGATCGCGATTTTTAATCTCTGAGCC (SEQ ID NO: 5768) |
| Gaussia luciferase | TTGTCCACCTGGCCCTGGATC (SEQ ID NO: 5769) |
| KRAS | TCATCAACACCCTGTCTTGTCTTTGCT (SEQ ID NO: 5770) |

TABLE 29
Priming sequences for site-specific amplification of RNA editing target
sites

| Editing site | Forward priming sequence | Reverse priming sequence |
|---|---|---|
| Cypridina luciferase | TAAACCAGGAAAAACATGTTGCC | CGCCCTTGGTTCCTTGACCC |
| X85W | (SEQ ID NO: 5771) | (SEQ ID NO: 5772) |
| STAT1 Y701C | AGGAAGCACCAGAGCCAATGGA | GGGGAGCAGGTTGTCTGTGGT |
| | (SEQ ID NO: 5773) | (SEQ ID NO: 5774) |
| CTNNB1 T41A/T41I | CTGATTTGATGGAGTTGGACATGGC | GTATCCACATCCTCTTCCTCAGGAT |
| | C | TGC (SEQ ID NO: 5776) |
| | (SEQ ID NO: 5775) | |
| STAT3 Y705C | CAGAGAGCCAGGAGCATCCTGA | TCTAAAGTGCGGGGGGACATCG |
| | (SEQ ID NO: 5777) | (SEQ ID NO: 5778) |
| LATS1 T1079A | GGAAAGCATCCTGAACATGCATTC | CGACTGCTGCTCTGAGCCTTG |
| | (SEQ ID NO: 5779) | (SEQ ID NO: 5780) |
| Gaussia luciferase | GCCAATGCCCGGAAAGCTGG | GGACTCTTTGTCGCCTTCGTAGGTG |
| C82R | (SEQ ID NO: 5781) | (SEQ ID NO: 5782) |
| KRAS D30D | AGAGAGGCCTGCTGAAAATGACTG | GAGAATATCCAAGAGACAGGTTTC |
| | (SEQ ID NO: 5783) | TCCATCA (SEQ ID NO: 5784) |
| KRAS L56L | TGGACGAATATGATCCAACAATAG | CTCATGTACTGGTCCCTCATTGCAC |
| | AGGATTC (SEQ ID NO: 5785) | TG (SEQ ID NO: 5786) |
| Illumina adapters | CTTTCCCTACACGACGCTCTTCCGA | GTTCAGACGTGTGCTCTTCCGATCT |
| | TCT (SEQ ID NO: 5787) | (SEQ ID NO: 5788) |

TABLE 30
Next-generation library preparation primers for sequencing of selected
ADAR2dd mutants

| Well | | |
|---|---|---|
| Position | Name | Sequence |
| A1 | Primer set | TCCCTACACGACGCTCTTCCGATCTCgatcGGGGGTTCCGGCggGtcc |
| | 1_Fwd_1 | (SEQ ID NO: 5789) |
| A2 | Primer set | TCCCTACACGACGCTCTTCCGATCTACgatcGGGGGTTCCGGCggGtcc |
| | 1_Fwd_2 | (SEQ ID NO: 5790) |
| A3 | Primer set | TCCCTACACGACGCTCTTCCGATCTGACgatcGGGGGTTCCGGCggGtcc |
| | 1_Fwd_3 | (SEQ ID NO: 5791) |
| A4 | Primer set | TCCCTACACGACGCTCTTCCGATCTTGACgatcGGGGGTTCCGGCggGtcc |
| | 1_Fwd_4 | (SEQ ID NO: 5792) |
| A5 | Primer set | TCCCTACACGACGCTCTTCCGATCTCTGACgatcGGGGGTTCCGGCggGtcc |
| | 1_Fwd_5 | (SEQ ID NO: 5793) |
| A6 | Primer set | TCCCTACACGACGCTCTTCCGATCTACTGACgatcGGGGGTTCCGGCggGtcc |
| | 1_Fwd_6 | (SEQ ID NO: 5794) |
| A7 | Primer set | TCCCTACACGACGCTCTTCCGATCTTACTGACgatcGGGGGTTCCGGCggGtcc |
| | 1_Fwd_7 | (SEQ ID NO: 5795) |
| A8 | Primer set | TCCCTACACGACGCTCTTCCGATCTGTACTGACgatcGGGGGTTCCGGCggGtcc |
| | 1_Fwd_8 | (SEQ ID NO: 5796) |
| B1 | Primer set | TCCCTACACGACGCTCTTCCGATCTCCGGGAACCAAATGCATTAACGGCGAA |
| | 2_Fwd_1 | (SEQ ID NO: 5797) |
| B2 | Primer set | TCCCTACACGACGCTCTTCCGATCTACCGGGAACCAAATGCATTAACGGCGA |
| | 2_Fwd_2 | (SEQ ID NO: 5798)A |
| B3 | Primer set | TCCCTACACGACGCTCTTCCGATCTGACCGGGAACCAAATGCATTAACGGCGA |
| | 2_Fwd_3 | A (SEQ ID NO: 5799) |
| B4 | Primer set | TCCCTACACGACGCTCTTCCGATCTTGACCGGGAACCAAATGCATTAACGGCG |
| | 2_Fwd_4 | AA (SEQ ID NO: 5800) |
| B5 | Primer set | TCCCTACACGACGCTCTTCCGATCTCTGACCGGGAACCAAATGCATTAACGGC |
| | 2_Fwd_5 | GAA (SEQ ID NO: 5801) |
| B6 | Primer set | TCCCTACACGACGCTCTTCCGATCTACTGACCGGGAACCAAATGCATTAACGG |
| | 2_Fwd_6 | CGAA (SEQ ID NO: 5802) |
| B7 | Primer set | TCCCTACACGACGCTCTTCCGATCTTACTGACCGGGAACCAAATGCATTAACG |
| | 2_Fwd_7 | GCGAA (SEQ ID NO: 5803) |
| B8 | Primer set | TCCCTACACGACGCTCTTCCGATCTGTACTGACCGGGAACCAAATGCATTAAC |
| | 2_Fwd_8 | GGCGAA (SEQ ID NO: 5804) |
| C1 | Primer set | TCCCTACACGACGCTCTTCCGATCTCCAATTCCACCTGTACATCTCCACATCA |
| | 3_Fwd_1 | (SEQ ID NO: 5805) |
| C2 | Primer set | TCCCTACACGACGCTCTTCCGATCTACCAATTCCACCTGTACATCTCCACATCA |
| | 3_Fwd_2 | (SEQ ID NO: 5806) |
| C3 | Primer set | TCCCTACACGACGCTCTTCCGATCTGACCAATTCCACCTGTACATCTCCACATC |
| | 3_Fwd_3 | A (SEQ ID NO: 5807) |
| C4 | Primer set | TCCCTACACGACGCTCTTCCGATCTTGACCAATTCCACCTGTACATCTCCACAT |
| | 3_Fwd_4 | CA (SEQ ID NO: 5808) |
| C5 | Primer set | TCCCTACACGACGCTCTTCCGATCTCTGACCAATTCCACCTGTACATCTCCACA |
| | 3_Fwd_5 | TCA (SEQ ID NO: 5809) |
| C6 | Primer set | TCCCTACACGACGCTCTTCCGATCTACTGACCAATTCCACCTGTACATCTCCAC |
| | 3_Fwd_6 | ATCA (SEQ ID NO: 5810) |
| C7 | Primer set | TCCCTACACGACGCTCTTCCGATCTTACTGACCAATTCCACCTGTACATCTCCA |
| | 3_Fwd_7 | CATCA (SEQ ID NO: 5811) |
| C8 | Primer set | TCCCTACACGACGCTCTTCCGATCTGTACTGACCAATTCCACCTGTACATCTCC |
| | 3_Fwd_8 | ACATCA (SEQ ID NO: 5812) |
| D1 | Primer set | TCCCTACACGACGCTCTTCCGATCTCCAGGGCGAAAGATTACTAACGATGAGC |
| | 4_Fwd_1 | (SEQ ID NO: 5813) |
| D2 | Primer set | TCCCTACACGACGCTCTTCCGATCTACCAGGGCGAAAGATTACTAACGATGAG |
| | 4_Fwd_2 | C |
| | | (SEQ ID NO: 5814) |
| D3 | Primer set | TCCCTACACGACGCTCTTCCGATCTGACCAGGGCGAAAGATTACTAACGATGA |
| | 4_Fwd_3 | GC |
| | | (SEQ ID NO: 5815) |
| D4 | Primer set | TCCCTACACGACGCTCTTCCGATCTTGACCAGGGCGAAAGATTACTAACGATG |
| | 4_Fwd_4 | AGC (SE QID NO: 5816) |
| D5 | Primer set | TCCCTACACGACGCTCTTCCGATCTCTGACCAGGGCGAAAGATTACTAACGAT |
| | 4_Fwd_5 | GAGC (SEQ ID NO: 5817) |
| D6 | Primer set | TCCCTACACGACGCTCTTCCGATCTACTGACCAGGGCGAAAGATTACTAACGA |
| | 4_Fwd_6 | TGAGC (SEQ ID NO: 5818) |
| D7 | Primer set | TCCCTACACGACGCTCTTCCGATCTTACTGACCAGGGCGAAAGATTACTAACG |
| | 4_Fwd_7 | ATGAGC (SEQ ID NO: 5819) |
| D8 | Primer set | TCCCTACACGACGCTCTTCCGATCTGTACTGACCAGGGCGAAAGATTACTAAC |
| | 4_Fwd_8 | GATGAGC (SEQ ID NO: 5820) |
| E1 | Primer set | TCCCTACACGACGCTCTTCCGATCTCCCCCCGTTGTACACTCTTAACAAACCA |
| | 5_Fwd_1 | (SEQ ID NO: 5821) |
| E2 | Primer set | TCCCTACACGACGCTCTTCCGATCTACCCCCCGTTGTACACTCTTAACAAACCA |
| | 5_Fwd_2 | (SEQ ID NO: 5822) |
| E3 | Primer set | TCCCTACACGACGCTCTTCCGATCTGACCCCCCGTTGTACACTCTTAACAAACC |
| | 5_Fwd_3 | A |
| | | (SEQ ID NO: 5823) |
| E4 | Primer set | TCCCTACACGACGCTCTTCCGATCTTGACCCCCCGTTGTACACTCTTAACAAAC |
| | 5_Fwd_4 | CA |
| | | (SEQ ID NO: 5824) |
| E5 | Primer set | TCCCTACACGACGCTCTTCCGATCTCTGACCCCCCGTTGTACACTCTTAACAAA |
| | 5_Fwd_5 | CCA (SEQ ID NO: 5825) |
| E6 | Primer set | TCCCTACACGACGCTCTTCCGATCTACTGACCCCCCGTTGTACACTCTTAACAA |
| | 5_Fwd_6 | ACCA (SEQ ID NO: 5826) |
| E7 | Primer set | TCCCTACACGACGCTCTTCCGATCTTACTGACCCCCCGTTGTACACTCTTAACA |
| | 5_Fwd_7 | AACCA (SEQ ID NO: 5827) |
| E8 | Primer set | TCCCTACACGACGCTCTTCCGATCTGTACTGACCCCCCGTTGTACACTCTTAAC |
| | 5_Fwd_8 | AAACCA (SEQ ID NO: 5828) |
| F1 | Primer set | TCCCTACACGACGCTCTTCCGATCTCAAGCACGCCTTATACTGCAGATGGATG |
| | 6_Fwd_1 | (SEQ ID NO: 5829) |
| F2 | Primer set | TCCCTACACGACGCTCTTCCGATCTACAAGCACGCCTTATACTGCAGATGGAT |
| | 6_Fwd_2 | G |
| | | (SEQ ID NO: 5830) |
| F3 | Primer set | TCCCTACACGACGCTCTTCCGATCTGACAAGCACGCCTTATACTGCAGATGGA |
| | 6_Fwd_3 | TG |
| | | (SEQ ID NO: 5831) |
| F4 | Primer set | TCCCTACACGACGCTCTTCCGATCTTGACAAGCACGCCTTATACTGCAGATGG |
| | 6_Fwd_4 | AT |
| | | G (SEQ ID NO: 5832) |
| F5 | Primer set | TCCCTACACGACGCTCTTCCGATCTCTGACAAGCACGCCTTATACTGCAGATG |
| | 6_Fwd_5 | GATG (SEQ ID NO: 5833) |
| F6 | Primer set | TCCCTACACGACGCTCTTCCGATCTACTGACAAGCACGCCTTATACTGCAGAT |
| | 6_Fwd_6 | GGATG (SEQ ID NO: 5834) |
| F7 | Primer set | TCCCTACACGACGCTCTTCCGATCTTACTGACAAGCACGCCTTATACTGCAGA |
| | 6_Fwd_7 | TGGATG (SEQ ID NO: 5835) |
| F8 | Primer set | TCCCTACACGACGCTCTTCCGATCTGTACTGACAAGCACGCCTTATACTGCAG |
| | 6_Fwd_8 | ATGGATG (SEQ ID NO: 5836) |
| A9 | Primer set | AGTTCAGACGTGTGCTCTTCCGATCTCCGTTCAGTGCTAGTCCCCTGTCACT |
| | 1_Rev_1 | (SEQ ID NO: 5837) |
| A10 | Primer set | AGTTCAGACGTGTGCTCTTCCGATCTACCGTTCAGTGCTAGTCCCCTGTCACT |
| | 1_Rev_2 | (SEQ ID NO: 5838) |
| A11 | Primer set | AGTTCAGACGTGTGCTCTTCCGATCTGACCGTTCAGTGCTAGTCCCCTGTCACT |
| | 1_Rev_3 | (SEQ ID NO: 5839) |
| A12 | Primer set | AGTTCAGACGTGTGCTCTTCCGATCTTGACCGTTCAGTGCTAGTCCCCTGTCAC |
| | 1_Rev_4 | T |
| | | (SEQ ID NO: 5840) |
| B9 | Primer set | AGTTCAGACGTGTGCTCTTCCGATCTCATACGGGCGTCACCACACGG |
| | 2_Rev_1 | (SEQIDNO: 5841) |
| B10 | Primer set | AGTTCAGACGTGTGCTCTTCCGATCTACATACGGGCGTCACCACACGG |
| | 2_Rev_2 | (SEQ ID NO: 5842) |
| B11 | Primer set | AGTTCAGACGTGTGCTCTTCCGATCTGACATACGGGCGTCACCACACGG |
| | 2_Rev_3 | (SEQ ID NO: 5843) |
| B12 | Primer set | AGTTCAGACGTGTGCTCTTCCGATCTTGACATACGGGCGTCACCACACGG |
| | 2_Rev_4 | (SEQ ID NO: 5844) |
| C9 | Primer set | AGTTCAGACGTGTGCTCTTCCGATCTCCCACCTCGCGATCTTGTCGGA |
| | 3_Rev_1 | (SEQ ID NO: 5845) |
| C10 | Primer set | AGTTCAGACGTGTGCTCTTCCGATCTACCCACCTCGCGATCTTGTCGGA |
| | 3_Rev_2 | (SEQ ID NO: 5846) |
| C11 | Primer set | AGTTCAGACGTGTGCTCTTCCGATCTGACCCACCTCGCGATCTTGTCGGA |
| | 3_Rev_3 | (SEQ ID NO: 5847) |
| C12 | Primer set | AGTTCAGACGTGTGCTCTTCCGATCTTGACCCACCTCGCGATCTTGTCGGA |
| | 3_Rev_4 | (SEQ ID NO: 5848) |
| D9 | Primer set | AGTTCAGACGTGTGCTCTTCCGATCTCCTTGCCTCGGCATTGGAAATCCC |
| | 4_Rev_1 | (SEQ ID NO: 5849) |
| D10 | Primer set | AGTTCAGACGTGTGCTCTTCCGATCTACCTTGCCTCGGCATTGGAAATCCC |
| | 4_Rev_2 | (SEQ ID NO: 5850) |
| D11 | Primer set | AGTTCAGACGTGTGCTCTTCCGATCTGACCTTGCCTCGGCATTGGAAATCCC |
| | 4_Rev_3 | (SEQ ID NO: 5851) |
| D12 | Primer set | AGTTCAGACGTGTGCTCTTCCGATCTTGACCTTGCCTCGGCATTGGAAATCCC |
| | 4_Rev_4 | (SEQ ID NO: 5852) |
| E9 | Primer set | AGTTCAGACGTGTGCTCTTCCGATCTCCTAAGCAGATGGGATGGTACTTTACC |
| | 5_Rev_1 | GTG (SEQ ID NO: 5853) |
| E10 | Primer set | AGTTCAGACGTGTGCTCTTCCGATCTACCTAAGCAGATGGGATGGTACTTTAC |
| | 5_Rev_2 | CGTG (SEQ ID NO: 5854) |
| E11 | Primer set | AGTTCAGACGTGTGCTCTTCCGATCTGACCTAAGCAGATGGGATGGTACTTTA |
| | 5_Rev_3 | CCGTG (SEQ ID NO: 5855) |
| E12 | Primer set | AGTTCAGACGTGTGCTCTTCCGATCTTGACCTAAGCAGATGGGATGGTACTTT |
| | 5_Rev_4 | ACCGTG (SEQ ID NO: 5856) |
| F9 | Primer set | AGTTCAGACGTGTGCTCTTCCGATCTCattcgccAcgTgagtctaggatcc |
| | 6_Rev_1 | (SEQ ID NO: 5857) |
| F10 | Primer set | AGTTCAGACGTGTGCTCTTCCGATCTACattcgccAcgTgagtctaggatcc |
| | 6_Rev_2 | (SEQ ID NO: 5858) |
| F11 | Primer set | AGTTCAGACGTGTGCTCTTCCGATCTGACattcgccAcgTgagtctaggatcc |
| | 6_Rev_3 | (SEQ ID NO: 5859) |
| F12 | Primer set | AGTTCAGACGTGTGCTCTTCCGATCTTGACattcgccAcgTgagtctaggatcc |
| | 6_Rev_4 | (SEQ ID NO: 5860) |

TABLE 31
Plasmids used in this study

| | | Expression | |
|---|---|---|---|
| Name | Description | system | Link to map |
| pAB1865 | pACYC184 pJ23119-BsmbI-B-t1 DR | Bacterial | |
| pAB1866 | pACYC184 pJ23119-BsmbI-B-t2 DR | Bacterial | |
| pAB1867 | pACYC184 pJ23119-BsmbI-B-t3 DR | Bacterial | |
| pAB1870 | pACYC184 pJ23119-BsmbI-B-t4 DR | Bacterial | |
| pAB1869 | pACYC184 pJ23119-BsmbI-B-t5 DR | Bacterial | |
| pAB1898 | pBR322 pLac-Cas13b-t1 | Bacterial | |
| pAB1899 | pBR322 pLac-Cas13b-t2 | Bacterial | |
| pAB1900 | pBR322 pLac-Cas13b-t3 | Bacterial | |
| pAB1903 | pBR322 pLac-Cas13b-t4 | Bacterial | |
| pAB1902 | pBR322 pLac-Cas13b-t5 | Bacterial | |
| pAB1619 | U6-BpiI-Cas13b-t1-DR | Mammalian | |
| pAB1620 | U6-BpiI-Cas13b-t3-DR | Mammalian | |
| pAB1853 | U6-BpiI-Cas13b-t5-DR | Mammalian | |
| pAB1678 | CMV-HIVNES-GS-Cas13b-t1 | Mammalian | |
| pAB1679 | CMV-HIVNES-GS-Cas13b-t3 | Mammalian | |
| pAB1891 | CMV-HIVNES-GS-Cas13b-t5 | Mammalian | |
| pAB1680 | CMV-HIVNES-GS-dCas13b-t1 | Mammalian | |
| pAB1681 | CMV-HIVNES-GS-dCas13b-t3 | Mammalian | |
| pAB1676 | CMV-HIVNES-GS-dCas13bt1-(GGS)2-huADAR2dd(E488Q) | Mammalian | |
| pAB1677 | CMV-HIVNES-GS-dCas13bt3-(GGS)2-huADAR2dd(E488Q) | Mammalian | |
| pAB1322 | CMV-HIVNES-GS-dCas13b6-(GGS)2-huADAR2dd(E488Q) | Mammalian | |
| pAB1659 | CMV-HIVNES-GS-dCas13b6-(GGS)2-huADAR2dd(E488Q/E620G) | Mammalian | |
| pAB1810 | CMV-HIVNES-GS-dCas13b6-(GGS)2-huADAR2dd(E488Q/E620G/Q696L) | Mammalian | |
| pAB1923 | CMV-HIVNES-GS-dCas13bt1-(GGS)2-huADAR2dd(E488Q/E620G/Q696L) | Mammalian | |
| pAB0040 | CMV-Cluciferase(STOP85)-polyAEF1a-G-luciferase-polyA | Mammalian | Previously |
| | | | described⁹ |
| pAB1424 | CMV-Cluciferase(W113XTGA)-polyA EF1a-G-luciferase-polyA | Mammalian | |
| pAB1425 | CMV-Cluciferase-polyAEF1a-G-luciferase(C73Y)-polyA | Mammalian | |
| pAB1588 | CMV-Cluciferase-polyAEF1a-G-luciferase(R93K)-polyA | Mammalian | |
| pAB1761 | CMV-Cluciferase-polyA EF1a-G-luciferase(R93Q CAA)-polyA | Mammalian | |
| pAB1763 | CMV-Cluciferase-polyA EF1a-G-luciferase(R93H CAT)-polyA | Mammalian | |
| pAB1764 | CMV-Cluciferase-polyA EF1a-G-luciferase(G92R GAG)-polyA | Mammalian | |
| pAB1456 | pYES3/CT pADH1-HH-ADE2 T(30 bp/22 mm)-B6-DR-HDV-ADH1-term | Yeast | |
| | TGA-ADE2 TAG-URA3 | | |
| pAB1417 | pGAL-dCas13b6-(GGS)2-dADAR2(E488Q) | Yeast | |
| pAB1773 | pGAL-dCas13b6-(GGS)2-dADAR2(E488Q/E620G) | Yeast | |

### REFERENCES

1. Abudayyeh, O. O. et al. Science 365, 382-386 (2019).
2. Chee, M. K. & Haase, S. B. G3 2, 515-526 (2012).
3. Voth, W. P., Jiang, Y. W. & Stillman, D. J. Yeast 20, 985-993 (2003).
4. Joung, J. et al. Nat. Protoc. 12, 828-863 (2017).
5. Gietz, R. D. & Schiestl, R. H. Nat. Protoc. 2, 31-34 (2007).
6. Gietz, R. D. & Schiestl, R. H. Nat. Protoc. 2, 38-41 (2007).
7. Matthews, M. M. et al. Nat. Struct. Mol. Biol. 23, 426-433 (2016).
8. Eggington, J. M., Greene, T. & Bass, B. L. Nat. Commun. 2, 319 (2011).
9. Cox, D. B. T. et al. Science 358, 1019-1027 (2017).

### Example 4—Small Cas13 Proteins Enable Compact RNA Base Editors

Applicants identified and characterized an ultra-small family of Cas13b, Cas13b-t, and showed it mediates mammalian transcript knockdown. By functionalizing Cas13b-t with adenosine and cytosine deaminase domains, Applicants engineered compact variants of REPAIR and RESCUE RNA editors, which may be more amenable for in vivo use. The systems here may be used for precise RNA editing as an attractive therapeutic strategy, e.g., when temporary changes are desirable or DNA editing is not possible.

RNA-targeting CRISPR-Cas13 systems have been harnessed for a variety of applications (1), including precision base editing (2, 3). RNA base editing is a promising therapeutic strategy that allows for installation of temporary, non-heritable edits. However, in some cases, therapeutic delivery of Cas13-based RNA editing systems remains challenging, in part because the size of cas13 genes identified so far exceed the packaging capacity of adeno-associated virus (AAV), the most widely used viral vector for gene delivery (4, 5).

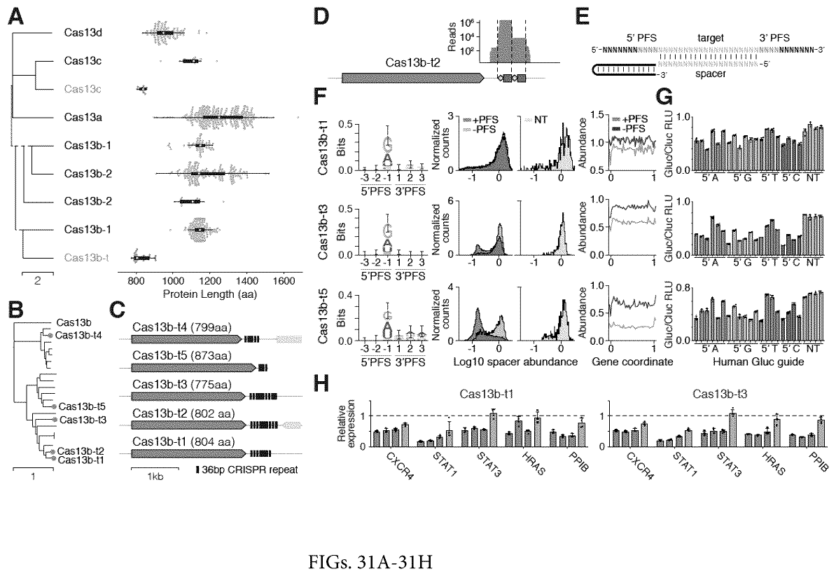

To overcome this limitation, Applicants performed a computational search of prokaryotic and viral genomes and metagenomes for small Cas13 orthologs, identifying 4726 candidates. Phylogenetic analysis revealed two novel groups of ultra-small Cas13 proteins that form distinct branches within the Cas13b and c subtypes. (FIG. 31A). Unlike other Type VI-B CRISPR-Cas loci6, the genomic loci encoding Cas13b-t lack any accessory genes. Applicants focused on the new tiny Cas13b (Cas13b-t) subfamily (FIG. 31B) in this example.

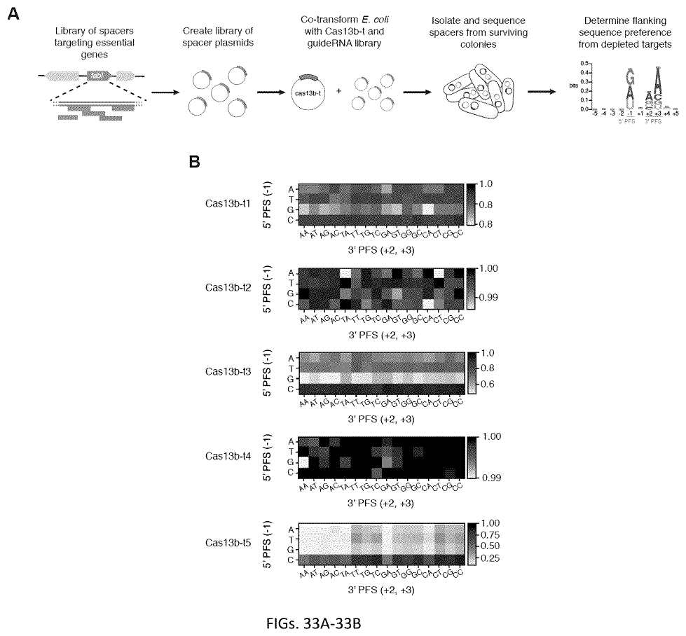

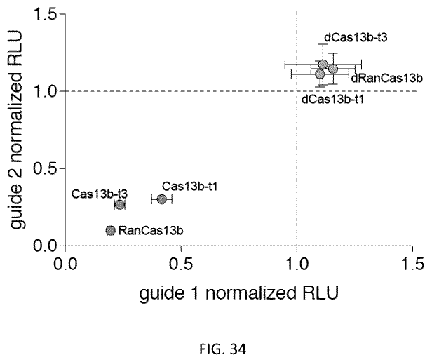

To experimentally characterize Cas13b-t, Applicants first identified the required CRISPR RNA (crRNA) components. Applicants transformed E. coli with a plasmid containing the Cas13b-t2 locus (FIGS. 31B-31C) with the CRISPR array truncated to two direct repeats (DRs) and performed small RNA sequencing. Applicants found that the crRNA of Cas13b-t2 has a 3′ DR (FIG. 31D). To determine if Cas13b-t is capable of mediating nucleic acid interference, Applicants performed a negative selection screen using a library of crRNAs that consist of a spacer followed by the DR and target essential gene transcripts in E. coli (6) (FIG. 33A). Three of the five tested members of the Cas13b-t subfamily, Cas13b-t1, 3, and 5, mediate depletion of targeting spacers in E. coli (FIG. 31F). Mapping of depleted spacers to the E. coli transcriptome and analysis of the flanking sequences revealed that all three active orthologs have a permissive 5′ D (A/G/T) protospacer flanking sequence (PFS) preference (FIG. 31F and FIG. 33B). Additionally, assessment of the normalized position of depleted spacers along the target transcript indicates no positional preference within the coding region and enhanced depletion when targeting the 5′ UTR (FIG. 31F).

To evaluate Cas13b-t-mediated knockdown and the importance of the PFS for RNA targeting in human cells, Applicants tested the three active Cas13b-t's using a set of 20 guide RNAs (gRNAs) with spacer sequences targeting regions with different adjacent 5′ bases in a Gaussia luciferase reporter. Applicants found that all three proteins promoted knockdown in HEK293FT cells with varying efficiencies, from 50% to 75% for the most efficient gRNA tested (FIG. 31G). Mutation of the HEPN domains in Cas13b-t1 and 3 (dCas13b-t1 and 3) abolished the knockdown activity (34). Further, Applicants found that the PFS preference detected in E. coli was not manifested in HEK293FT cells, indicating that the PFS has little effect in mammalian cells, similar to previously studied Cas13's (2) (FIG. 31G). Applicants next targeted endogenous transcripts in mammalian cells with Cas13b-t1 and 3, the smallest and most active members of the tested Cas13b-t's. Both proteins mediated knockdown of five target transcripts for all gRNAs tested (12-68% and 27-64% knockdown compared to a non-targeting gRNA for Cas13b-t1 and 3, respectively) (FIG. 31H).

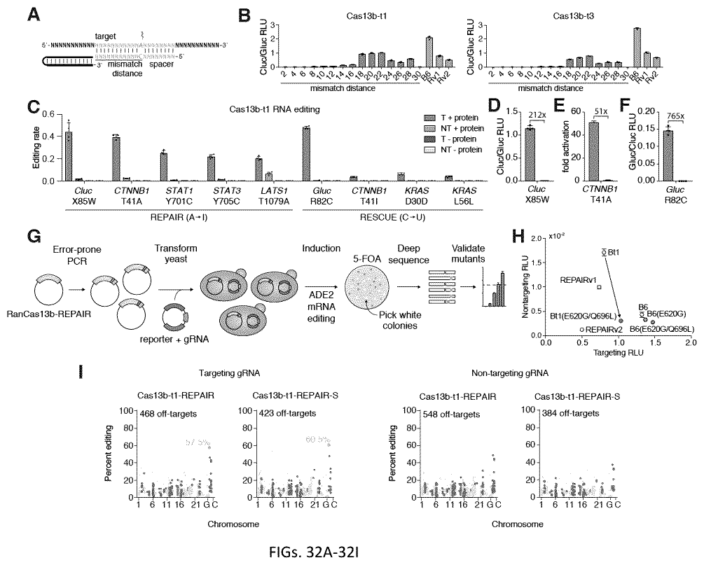

To test the capacity of Cas13b-t's for RNA editing, Applicants fused dCas13b-t1 and 3 with a hyperactive mutant of the human adenosine deaminase acting on RNA 2 (ADAR2dd(E488Q)) to create Cas13b-t1-REPAIR and Cas13b-t3 REPAIR. Applicants evaluated the ability of these fusion proteins to direct A-to-I RNA editing in HEK293FT cells by attempting to revert tryptophan (W) 85 to STOP (X) mutation in a Cypridina luciferase reporter. Site-specific RNA editing is achieved by introducing a cytidine mismatch in the gRNA spacer sequence across from the target adenosine2,7 (FIG. 32A). Spacer sequences were designed to vary the distance between this mismatch and the DR, as variability in the optional mismatch position has been observed for different Cas13b-ADAR fusion proteins and target sites (2, 3). Applicants found that both Cas13b-t1-REPAIR and Cas13b-t3-REPAIR showed optimal editing with a mismatch distance of 18-22 base pairs (bp) in a 30-bp spacer sequence. Editing efficiency was comparable to the previously described REPAIRv1 and v2 systems (2) and approximately 50% and 13% of that of the more efficient RanCas13b-REPAIR (3) for Cas13b-t1-REPAIR and Cas13b-t3-REPAIR, respectively (FIG. 32B).

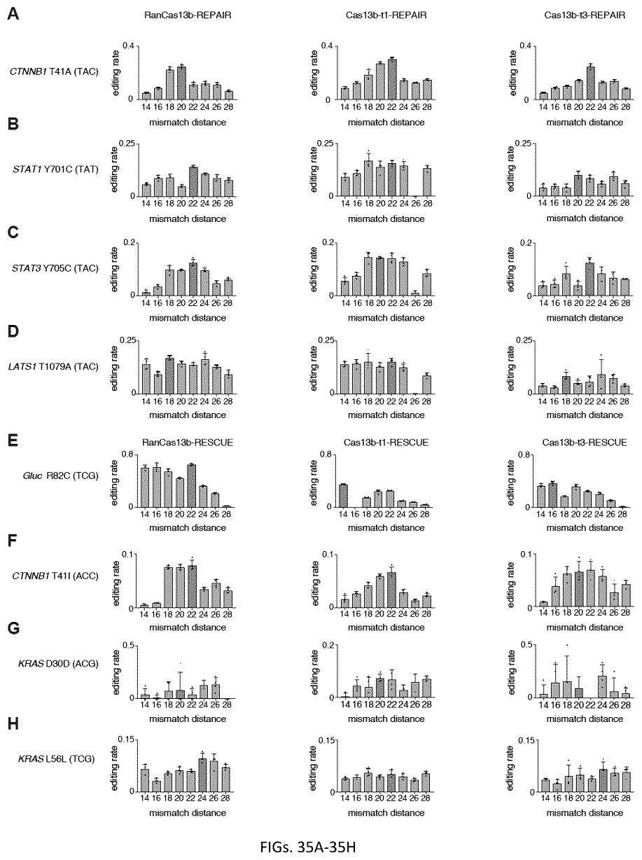

Applicants additionally fused both dCas13b-t1 and dCas13b-t3 with a previously described evolved ADAR2dd capable of cytidine to uridine deamination3 (Cas13b-t1-RESCUE and Cas13b-t3-RESCUE) and directed both editors to reporter and endogenous transcripts in HEK293FT cells (FIGS. 35A-35H). Applicants found that these fusion proteins were capable of mediating both A-to-I or C-to-U editing of all targets tested at levels comparable to or better than RanCas13b-REPAIR/RESCUE (FIGS. 32C-32F and FIGS. 36A-36L).

To demonstrate the ability of Cas13b-t-REPAIR to edit functionally relevant targets, Applicants targeted previously characterized phosphorylation sites. In particular, Applicants attempted to alter activation of the Wnt/beta-catenin pathway by editing the threonine (T) 41 codon of CTNNB1, a site known to promote degradation of beta-catenin when phosphorylated (8). Applicants found that Cas13b-t1-REPAIR was able to mediate 40% editing at this site, converting the codon to alanine (A) and leading to a 51-fold increase in beta catenin activity, which may be relevant for promoting regeneration after acute liver failure (9, 10) (FIG. 32E). Cas13b-t1-REPAIR was also able to efficiently edit sites corresponding to phosphorylated residues in the STAT1, STAT3 and LATS1 transcripts (FIG. 32C).

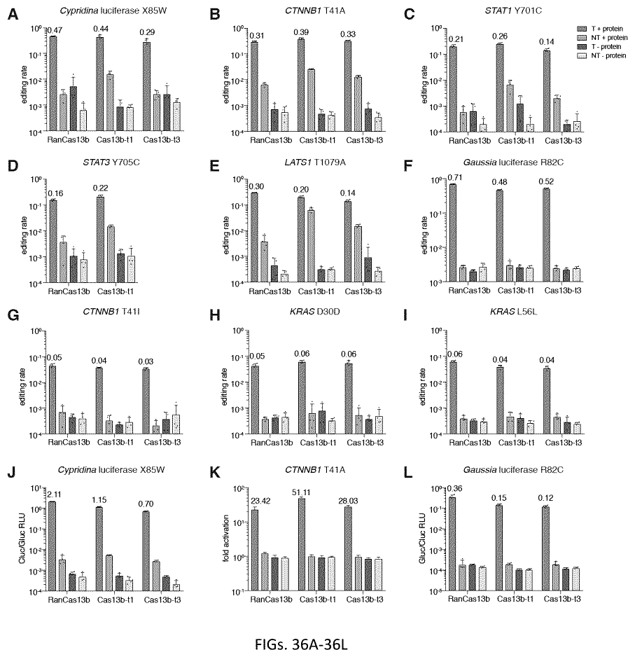

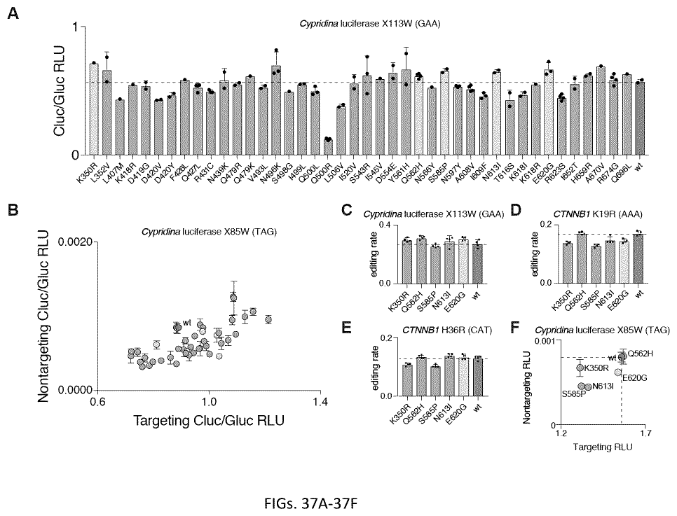

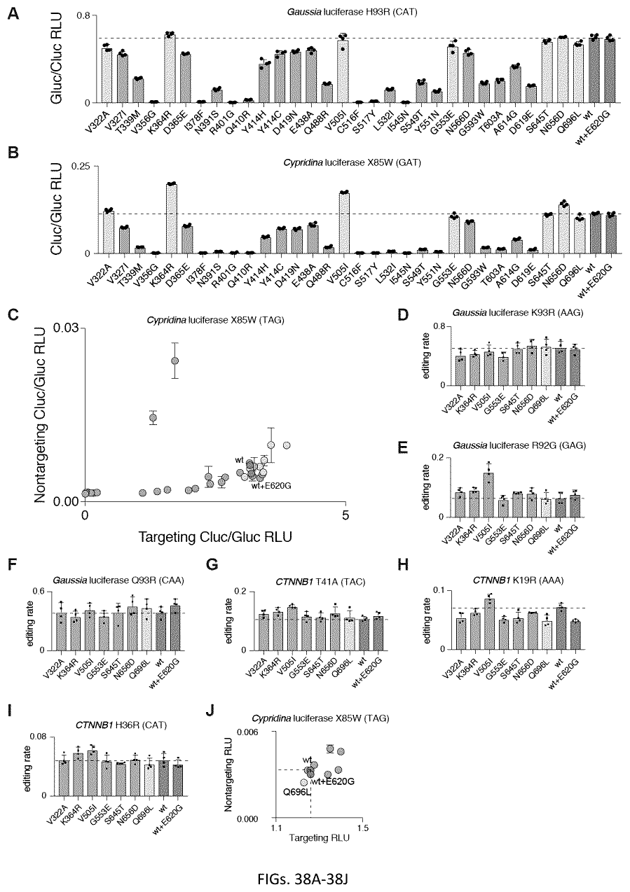

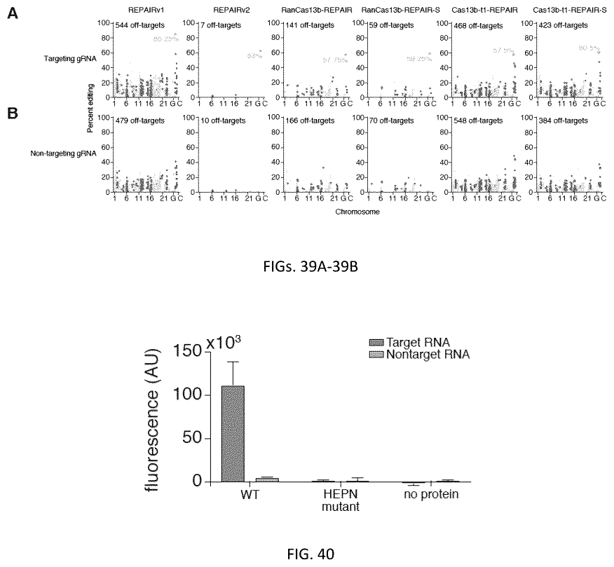

Finally, Applicants evaluated the transcriptome-wide specificity of Cas13b-t1-REPAIR and found the number of off-target edits caused by this system was comparable to REPAIRv1 (FIGS. 39A-39B), which may be due to promiscuous activity of the ADAR deaminase domain (2, 3). To additionally accelerate the translation of REPAIR to therapeutic use, Applicants sought to improve the specificity of Cas13b-t1-REPAIR (FIG. 32G). Although higher specificity ADAR2dd variants have been engineered, they substantially reduced editing efficiency (2). Through a parallel effort to directly evolve ADAR mutants that are both highly specific and efficient in the context of fusion with dRanCas13b, Applicants identified two promising mutations in ADAR2dd (E620G and Q696L) (FIGS. 37A-37F, 38A-38J). Applicants incorporated these two mutations in Cas13b-t1-REPAIR and found that the number of off-target edits decreased while maintaining comparable on-target activity as the original Cas13b-t1-REPAIR (FIGS. 32H-32I).

The small size and high efficacy of Cas13b-t-REPAIR and RESCUE constructs makes them compatible with viral delivery, resolving a major challenge to deployment of this novel therapeutic strategy.

### Methods

### Data Curation and Search Pipeline

Assembled prokaryotic and phage genomic DNA contigs from metagenomes and genomes were downloaded from NCBI, WGS, and JGI, totaling 3.16 trillion bp. All open reading frames larger than 80 aa were annotated resulting in 10 billion putative proteins for further analysis. Previously developed Cas13 profiles 11 were used to identify Cas13 family proteins with HMMER3.212 using a minimum bitscore threshold of 25. A group of small (˜800aa) but divergent Cas13b's were identified and used to seed a second HMMER search with the same settings to retrieve additional members of this subfamily. In total, 4726 Cas13 proteins were identified.

### Phylogenetic Analysis

For phylogenetic analysis and classification, the 4726 candidate genes were clustered using MMseqs2 with a minimum sequence identity of 50% and minimum coverage of 70% (13, 14). Proteins within each cluster were clustered at 90% identity and 80% minimum coverage for redundancy reduction. Each redundancy reduced cluster was aligned using MAFFT (15) with default parameters. Proteins identified as truncated or partial and/or clusters entirely composed of them were removed from the analysis.

The aligned redundancy reduced clusters were converted into HHsuite profiles using all columns with less than 50% gaps, and each of these profiles was searched against each other with profile-profile alignment using HHsearch. The resulting pairwise bitscores between clusters, sij, where i,j denote clusters i and j, respectively, were used to construct a classification dendrogram. First, the asymmetric bitscores were symmetrized by setting sᵢⱼ=(sᵢⱼ+sⱼᵢ)/2. Then, pseudo-distances were calculated by setting dij=−(log sᵢⱼ−log min(sᵢᵢ, sⱼⱼ))/2 to generate a distance matrix (16). A UGPMA dendrogram was constructed using these distances. Branches and the subtrees of the dendrogram were contracted without modifying their topology, to highlight known subtypes and subgroups within each subtype. Lengths in amino acids (aa) of the redundancy reduced proteins from each subtree were used to generate protein size distributions.

### Design and Cloning of Bacterial Expression Plasmid Constructs

All cloning in this study was performed using chemically competent Stbl3 E. coli (NEB) unless otherwise noted. All PCR for cloning was performed using 2X Phusion Flash High-Fidelity Master Mix (Thermo Fisher) unless otherwise noted.

The Cas13b-t2 full locus was synthesized and cloned into the BamHI site of pACYC184 by GenScript.

To clone bacterial expression plasmids for the PFS screen, Cas13b-t protein coding sequences were human codon optimized using GeneArt GeneOptimizer (Thermo Fisher) and synthesized by GenScript into a pcDNA3.1(+) backbone. Genes were amplified by PCR to add a pLac promoter and cloned into a pBR322 backbone (NEB) digested with EcoRV (Thermo Fisher) by Gibson assembly.

crRNA expression cassettes for each DR corresponding to each Cas13b-t of interest were synthesized by IDT, amplified by PCR, and cloned into a pACYC184 backbone digested with EcoRV and BamHI (Thermo Fisher) by Gibson assembly. All primers are listed in Table 35 and final constructs in Table 46.

### Design and Cloning of Mammalian Expression Plasmid Constructs

Mammalian gRNA expression cassettes were amplified from pC0048 (Addgene plasmid #103854; n2t.net/addgene:103854; RRID:Addgene_103854)2 using primers to add the DR for each Cas13b-t ortholog of interest and cloned into pC0048 digested with LguI and KpnI (Thermo Fisher) using Gibson assembly.

Mammalian protein expression cassettes were cloned by amplifying previously mentioned synthesized Cas13b-t genes by PCR and cloning into pC0053 (Addgene plasmid #103869; n2t.net/addgene:103869; RRID:Addgene_l03869)2 digested with HindIII and NotI (Thermo Fisher), either alone or with addition of a piece including ADAR2dd(E488Q) amplified from pC0053 for REPAIR constructs and pC0078 (Addgene plasmid #130661; http://n2t.net/addgene:130661; RRID:Addgene_130661)3 for RESCUE constructs. Site directed mutagenesis was used to create catalytically inactivated Cas13b-t's. All primers are listed in Table 36 and final constructs in Table 46.

ADAR2 mutants derived from directed evolution screens were cloned by introduction of mutations via PCR primers.

gRNA spacers were cloned into expression backbones by Golden Gate assembly as previously described17. Spacer sequences are listed in Tables 40, 41, 43 and 44.

### Bacterial RNA Sequencing

Bacterial RNA sequencing was performed as previously described (18). Briefly, 5 mL overnight cultures of a Stbl3 E. coli colony transformed with a plasmid containing the locus of interest was spun down and resuspended in 1 mL of TRI Reagent (Zymo Research). After a 5-minute room temperature incubation, 250 μL of 0.5 mm Zirconia beads were added and the Trizol resuspension was vortexed vigorously for 30 s to 1 min. 200 uL chloroform was added, samples were inverted gently, incubated at room temperature for 3 minutes, and then spun down at 12000×g for 5 min at 4° C. Following centrifugation, the aqueous fraction was used as input to the Qiagen miRNeasy kit, as per the manufacturer's instructions.

Purified RNA was treated with DNase I (NEB), purified again using RNA Clean & Concentrate-25 (Zymo Research), and treated with T4 polynucleotide kinase (PNK) (NEB). PNK-treated RNA was again purified using RNA Clean & Concentrate-25 (Zymo Research), and ribosomal RNA was removed using the Ribominus Transcriptome Isolation Kit (Yeast and Bacteria) (Thermo Fisher Scientific). Samples were subsequently treated with RNA 5′ polyphosphatase (Epicentre) and purified again using an RNA Clean & Concentrate-5 kit (Zymo Research). Purified RNA was used as input to the NEBNext Multiplex Small RNA Library Prep Set for Illumina (NEB). Library preparation was performed as per the manufacturer's instructions, except with a final PCR of 20 cycles. Libraries were quantified by qPCR using the KAPA Library Quantification Kit for Illumina (Roche) on a StepOnePlus Real-Time PCR System (Thermo Fisher Scientific) and sequenced on an Illumina NextSeq. Reads were mapped using BWA and a custom Python script available upon request.

E. coli Essential Gene PFS Screen

Libraries were designed as previously described6. The library of spacers was cloned into each Cas13b-t pJ23119-spacer-DR backbone containing a chloramphenicol resistance gene using Golden Gate Assembly with a 5:1 ratio of spacer library to pre-digested backbone with 210 cycles. Libraries were transformed into Endura Electrocompetent Cells (Lucigen) by electroporation and plated over five 22.7 cm×22.7 cm chloramphenicol LB agar plates. 12 hours after plating, libraries were scraped from plates and DNA was extracted using the Macherey-Nagel Nucleobond Xtra Maxiprep Kit (Macherey-Nagel). 200 ng of library plasmid and 200 ng Cas13b-t gene plasmid containing an ampicillin resistance gene were transformed into 100 μL of Endura Electrocompetent Cells (Lucigen) by electroporation as per the manufacturer's protocol and plated across four 22.7 cm×22.7 cm ampicillin/chloramphenicol LB agar plates per biological replicate, with three biological replicates per condition. 10-12 hours post-transformation, libraries of transformants were scraped from the plates and DNA was extracted using the Macherey-Nagel Nucleobond Xtra Maxiprep Kit (Macherey-Nagel). Libraries were prepared from extracted DNA for next generation sequencing using primers in Supplementary Table 38 with NEBNext High-Fidelity 2X PCR Master Mix (NEB) and sequenced on an Illumina NextSeq. Spacer abundance relative to an empty vector was analyzed using a custom Python script, available on request. A mixed Gaussian distribution was fit to the distribution of negative control spacers, and the distribution with the higher mean was used as the null distribution. Depleted spacers were selected as those greater than 5 standard deviations away from the selected null distribution mean. Weblogos were generated using weblogo.berkeley.edu/logo.cgi using the top 1% of depleted spacers.

### Mammalian Cell Culture and Transfection

Mammalian cell culture experiments were performed in the HEK293FT line (American Type Culture Collection (ATCC)) grown in Dulbecco's Modified Eagle Medium with high glucose, sodium pyruvate, and GlutaMAX (Thermo Fisher Scientific), additionally supplemented with 1×penicillin-streptomycin (Thermo Fisher Scientific), 10 mM HEPES (Thermo Fisher Scientific), and 10% fetal bovine serum (VWR Seradigm). All cells were maintained at confluency below 80%.

All transfections were performed with Lipofectamine 2000 (Thermo Fisher Scientific) in 96-well plates. Cells were plated at approximately 20,000 cells/well 16-20 hours prior to transfection to ensure 90% confluency at the time of transfection. For each well on the plate, transfection plasmids were combined with OptiMEM I Reduced Serum Medium (Thermo Fisher Scientific) to a total of 25 μl. Separately, 24.5 μl of OptiMEM was combined with 0.5 μl of Lipofectamine 2000. Plasmid and Lipofectamine solutions were then combined and pipetted onto cells.

### Mammalian RNA Knockdown Assays

HEK293FT cells were transfected as described with 75 ng of a plasmid encoding expression of either a Cas13b-t ortholog or GFP from a CMV promoter, 150 ng of a plasmid encoding expression of a gRNA from a human U6 promoter and, where relevant, 45 ng of reporter plasmid. After 48 h, RNA was harvested as described previously17 with 2× the amount of recommended DNase and a 20 minute lysis step. RNA expression was measured by qPCR using commercially available TaqMan probes (Thermo Fisher Scientific) on a LightCycler 480 II (Roche) with GAPDH as an endogenous internal control in 5 uL multiplexed reactions17. Probes and primer sets were generally selected to amplify across the Cas13 target site so as to minimize detection of cleaved transcripts. Data is the average of 4 biological replicates with fold-change calculated relative to a negative control condition with the corresponding gRNA expression plasmid co-transfected with the GFP expression plasmid rather than a Cas13b-t expression plasmid using the ddCt method19. Error bars were calculated in GraphPad Prism 7 and represent the standard deviation, n=4.

For luciferase reporter assays, media was aspirated from cells and Cypridina and Gaussia luciferase activity in the media was measured using Gaussia and Cypridina Luciferase Assay Kits (Targeting Systems) with an injection protocol on a Biotek Synergy Neo 2 (Agilent). Each experimental luciferase measurement was normalized to the appropriate control luciferase measurement (i.e., if Cypridina luciferase was targeted, the Gaussia luciferase measurement was used as the control value and vice versa). For knockdown assays, normalized luciferase values were then again normalized to an average normalized luciferase measurement of 4 biological replicates of a negative control condition consisting of the corresponding gRNA expression plasmid co-transfected with a GFP expression plasmid rather than a Cas13 expression plasmid. Error bars were calculated in GraphPad Prism 7 and represent the standard deviation of the luciferase values normalized to negative control transfection, n=4.

### Mammalian RNA Editing Assays

HEK293FT cells were transfected as described with 150 ng a plasmid encoding expression of a dCas13b ortholog-ADAR2dd(E488Q) fusion from a CMV promoter, 300 ng of a plasmid encoding expression of a gRNA from a human U6 promoter and, where relevant, 45 ng of a reporter plasmid. After 48 h, RNA was harvested as described previously and reverse transcription was performed as described17 using gene-specific primers for the relevant target transcript (Table 19). cDNA was used as input for library preparation of next-generation sequencing libraries (Table 20) using NEBNext High-Fidelity 2X PCR Master Mix (NEB), and amplicons were sequenced on an Illumina MiSeq. Editing was quantified by counting the number of reads at which the expected edited position in the amplicon was called as a G (for A-to-I editing) or T (for C-to-U editing) and dividing by the total number of reads in the sample using a custom Python script, available upon request. Unless otherwise noted, all reported data is the average of 4 biological replicates.

Luciferase reporter assays for RNA editing were performed as described above, with the modification that normalized luciferase values were not normalized to a GFP control condition. For CTNNB1 targeting, Applicants engineered a luciferase reporter by replacing the EF1alpha promoter driving Gaussia luciferase expression in the dual luciferase reporter plasmid with a promoter derived either from an M50 Super 8× TOPFlash (TOP) or M51 Super 8× FOPFlash (FOP) reporter. M50 Super 8× TOPFlash (Addgene plasmid #12456; n2t.net/addgene:12456; RRID:Addgene_12456) and M51 Super 8× FOPFlash (TOPFlash mutant) (Addgene plasmid #12457; n2t.net/addgene:12457; RRID:Addgene_12457) were gifts from Randall Moon3,20. Luciferase activity was measured for these custom dual luciferase reporters for each protein/gRNA condition and normalized as described for a dual luciferase reporter. Fold activation was calculated by taking the ratio of the average TOP measurement and dividing by average FOP measurement, and error was calculated by a standard error propagation formula.

Optimal spacers for all target sites tested were determined by tiling spacers across the site of interest, varying the distance of the mismatch from the DR from 14 bp to 28 bp in intervals of 2 bp.

### RNA Editing Specificity

HEK293FT cells were transfected as described for mammalian RNA editing assays. After 48 h, RNA was harvested using a QIAGEN RNeasy Plus 96 kit as per the manufacturer's protocol. The mRNA fraction was enriched using an NEBNext Poly(A) Magnetic Isolation Module (NEB). Libraries were prepared using an NEBNExt Ultra II Directional RNA library prep kit (NEB) as per the manufacturer's protocol and sequenced on an Illumina NextSeq. Each sample was sequenced with an average read depth of 8 million reads per sample and randomly downsampled to 5 million reads per sample. Data was analyzed using a previously described custom pipeline on the FireCloud computational framework and downstream analysis using a custom Python script2,3. Any significant edits found in eGFP-transfected conditions were considered to be SNPs or artifacts of the transfection and filtered out. An additional layer of filtering for known SNP positions was performed using the Kaviar2l method for identifying SNPs.

### REFERENCES

1. Terns, M. P. Mol. Cell 72, 404-412 (2018).
2. Cox, D. B. T. et al. Science 358, 1019-1027 (2017).
3. Abudayyeh, O. O. et al. Science 365, 382-386 (2019).
4. Dong, J. Y., Fan, P. D. & Frizzell, R. A. Hum. Gene Ther. 7, 2101-2112 (1996).
5. Wu, Z., Yang, H. & Colosi, P. Mol. Ther. 18, 80-86 (2010).
6. Smargon, A. A. et al. Mol. Cell 65, 618-630.e7 (2017).
7. Matthews, M. M. et al. Nat. Struct. Mol. Biol. 23, 426-433 (2016).
8. MacDonald, B. T., Tamai, K. & He, X. Developmental Cell vol. 17 9-26 (2009).
9. Apte, U. et al. Am. J. Pathol. 175, 1056-1065 (2009).
10. Bhushan, B. et al. Am. J Pathol. 184, 3013-3025 (2014).
11. Shmakov, S. A., Makarova, K. S., Wolf, Y. I., Severinov, K. V. & Koonin, E. V. Proc. Natl. Acad. Sci. U.S.A 115, E5307-E5316 (2018).
12. Eddy, S. R. PLoS Comput. Biol. 7, e1002195 (2011).
13. Steinegger, M. & Söding, J. Nat. Biotechnol. 35, 1026-1028 (2017).
14. Steinegger, M. & Söding, J. Nat. Commun. 9, 2542 (2018).
15. Katoh, K. & Standley, D. M. Mol. Biol. Evol. 30, 772-780 (2013).
16. Makarova, K. S. et al. Nature Reviews Microbiology vol. 18 67-83 (2020).
17. Joung, J. et al. Nat. Protoc. 12, 828-863 (2017).
18. Zetsche, B. et al. Cell 163, 759-771 (2015).
19. Schmittgen, T. D. & Livak, K. J. Nature Protocols vol. 3 1101-1108 (2008).
20. Veeman, M. T., Slusarski, D. C., Kaykas, A., Louie, S. H. & Moon, R. T. Curr. Biol. 13, 680-685 (2003).
21. Glusman, G., Caballero, J., Mauldin, D. E., Hood, L. & Roach, J. C. Bioinformatics 27, 3216-3217 (2011).

### Additional Methods

### Protein Expression and Purification of Cas13b-t3

Wild-type and HEPN mutants were expressed from a pET28-based vector with an N-terminal TwinStrep-SUMO tag transformed into chemically competent Rosetta Competent Cells (Novagen/EMD Millipore). Cells transformed with the expression plasmid were grown in 1 L of Terrific Broth at 37° C. until OD 0.6. Temperature was switched to 18° C. and the cultures were induced with 0.2 mM IPTG. Cultures were grown for 16-18 h, then cells were harvested with centrifugation at 5000×g at 4° C. The pellets were resuspended in 150 mL lysis buffer (150 mM NaCl, 20 mM Tris-HCl pH 7.5, 1 mM DTT, 5% glycerol) and homogenized by mixing on a magnetic plate at 4° C. for 30 min. Cells were lysed by two passes through a microfluidizer at 18,000 psi and soluble fraction was separated from cell debris by centrifugation at 9,000 RPM for 30 min at 4° C. The soluble fraction was passed through Strep-Tactin resin (Qiagen). Resin was washed with 8 column volumes of lysis buffer and eluted from the column in lysis buffer supplemented with 5 mM desthiobiotin (Sigma). The tags were cleaved overnight at 4° C. by addition of SUMO protease. After cleavage, the proteins were passed through a heparin column (GE Healthcare) and concentrated to approximately 500 uL. Concentrated proteins were then passed through a Superdex 200 increase column (GE Healthcare) equilibrated in storage buffer (500 mM NaCl, 20 mM Tris-HCl pH 7.5, 1 mM DTT, 5% glycerol). Peak fractions were pooled and concentrated.

### Fluorescent Collateral RNA Cleavage Assay

Assays were carried out with 4 technical replicates with equimolar ratios of Cas13b-t3 wild-type or HEPN mutant protein, crRNA and RNA target in cleavage assay buffer (50 mM NaCl, 20 mM Tris-HCl pH 7.5, 5 mM MgCl2) with 10 U murine RNase inhibitor (New England Biolabs) and 500 nM RNAse Alert v2 sensor (Thermo Fisher). Samples were incubated for 3 hours at 37° C. on a fluorescent plate reader equipped with a FAM filter set. Measurements were taken at 5 minute intervals and data were normalized to the first time point.

### Design and Cloning of Yeast Expression Plasmid Constructs

Yeast reporter constructs were cloned into a pYES3/CT backbone (Thermo Fisher). A previously described reporter containing a crRNA expression cassette under a pADH1 terminator 1 was digested with HindIII and MluI (Thermo Fisher). A URA3 gene was amplified by PCR using the selection marker from a pRSII426 backbone2 with the introduced stop codon added by site-directed mutagenesis (Table 37) and cloned via Gibson assembly This backbone was digested with BcuI (Thermo Fisher) and an ADE2 gene amplified from M3499 ura3::ADE2 Disruptor Converter, a gift from David Stillman (Addgene plasmid #51674; n2t.net/addgene:51674; RRID:Addgene_51674)3, with the introduced stop codon added by site-directed mutagenesis (Table 37) and cloned via Gibson assembly. gRNA spacers were cloned into this backbone using Golden Gate assembly4.

Yeast REPAIR expression plasmids were derived from a previously described pRSII426 backbone2 with a pGAL promoter driving expression of the REPAIR fusion protein1. The URA3 selection marker was replaced with a LEU2 selection marker by digesting this backbone with Eco105I and KpnI (Thermo Fisher) and inserting a LEU2 gene amplified from a synthesized gene (IDT) by Gibson assembly. ADAR2 mutants to create sequences that could be used as a basis for error-prone PCR for each subsequent evolution round were inserted by amplifying the analogous sequence from the previous round of evolution and adding the new mutation via the site-directed mutagenesis (Table 37).

### Cloning of Mutagenesis Libraries for ADAR Evolution

ADAR2dd mutant libraries were generated by performing 8 error-prone PCR reactions for 20 cycles using a GeneMorph II Random Mutagenesis Kit (Agilent) with titrated template concentrations. For each round of evolution, we used a yeast codon-optimized ADAR2dd gene containing the selected mutants from all prior rounds. Resulting PCR reactions were pooled, gel purified, subjected to DpnI (Thermo Fisher) treatment and cloned into a yeast RanCas13b-REPAIR expression backbone (Supplementary Table 17) digested with KflI and Eco72I (Thermo Fisher) by Gibson assembly. Libraries were transformed into Endura Electrocompetent Cells (Lucigen) by electroporation and plated over one 22.7 cm×22.7 cm ampicillin LB agar plate. After 12-16 hours of growth, libraries were scraped from plates and DNA was extracted using the Macherey-Nagel Nucleobond Xtra Maxiprep Kit (Macherey-Nagel). Primers are listed in Table 37.

### Directed Evolution of High-Specificity ADAR Mutants

Applicants performed two rounds of evolution as follows:

To select for highly specific and efficient ADAR variants, Applicants engineered a yeast reporter based on simultaneous restoration of a TGA stop codon in ADE2 and negative selection of restoration of a TAG stop codon in URA3. We transformed Saccharomyces cerevisiae Meyen ex E. C. Hansen (ATCC® 204681™) with this plasmid, which also included expression of a crRNA targeting ADE2. Yeast were transformed using the lithium acetate/single-stranded carrier DNA/PEG method5.

Large scale transformations of mutagenesis libraries were performed as previously described (1, 6). Briefly, Applicants picked a colony from the initial transformation of the reporter plasmid, inoculated 300 mL of 2% glucose minimal media -tryptophan (Trp) for selection and grew overnight in a baffled flask at 30° C. After 12-16 hours of growth, Applicants measured the optical density (OD) of the culture and used this measurement to seed 2.5E9 cells into 500 mL of pre-warmed 2×YPAD media in a non-baffled flask. Once this culture reached an OD of 2 (approximately 4 hours), cells were harvested by centrifugation at 3000×g for 5 min, followed by two washes with water. The resulting cell pellet was then resuspended in 36 mL of transformation mix consisting of 24 mL of PEG 3350 (50% w/v), 3.6 mL of 1.0 M Lithium acetate, 5 mL of denatured single-stranded carrier salmon sperm DNA at 2.0 mg/mL (Thermo Fisher), 2.9 mL of water, and 500 μL of 1 μg/μL plasmid library. The mixture was incubated at 42° C. for 60 minutes with agitation, then the cells were pelleted once more and resuspended in 750 mL of 2% glucose minimal media -Trp/-leucine (Leu) and grown overnight at 30° C. in a baffled flask until OD reached between 6 and 8. 6.25 mL of the culture was then seeded into 250 mL of 2% raffinose -Trp/-Leu selection media and grown until OD reached between 0.5 and 1. The culture was then induced by adding 27 mL of 30% galactose and incubated overnight at 30° C. for 12-15 hours.

After overnight growth, cultures were plated across 20 22.7×22.7 cm selection plates of 2% raffinose/3% galactose -Trp/-Leu with 5 mg/L adenine (Ade) and 0.1% 5-fluoroorotic acid (5-FOA). After 2-3 days of selection, Applicants picked white colonies corresponding to an on-target edit and restoration of ADE2 and streaked these onto small selection plates of the same media base to ensure accurate colony picking. Plates were then allowed to grow again for up to 3 days. White streaks after this second selection were again picked.

To look for enriched single mutations, all picked streaks were pooled and the contained RanCas13b-REPAIR genes were amplified with NEBNext High-Fidelity 2X PCR Master Mix (NEB) for preparation of next generation sequencing libraries. Libraries were sequenced on an Illumina NextSeq. Relative enrichment of mutations in the selected library was analyzed using a custom Python script, available upon request. Identified enriched single mutants were introduced by site-directed mutagenesis to RanCas13b-REPAIR in mammalian expression vectors for validation (Table 38).

To test the candidate mutations, RNA editing assays using luciferase reporters in HEK293FT cells were performed as previously described. Specifically, after the first round of selection, RanCas13b-ADAR2dd mutants were targeted to either of 2 Cypridina luciferase reporters, one with a W85X mutation (TAG stop codon) and one with a W13X mutation (TGA stop codon) to evaluate the ability of the evolved ADAR2dd's to effectively edit at sites with both preferred and non-preferred 5′ bases7,8 (FIGS. 37A-37B). After the second round of evolution, this initial screening was performed using the same Cypridina luciferase W85X reporter, along with a second Cypridina luciferase W85X (TGA stop codon) reporter and a Gaussia luciferase R93H reporter for which restoration of a CAT codon to CGT reverts a catalytically-inactivating mutation (FIGS. 38A-38C). Luciferase activity of the Cypridina luciferase W85X TAG reporter in the non-targeting crRNA condition was also used as a proxy for measuring specificity, as previously described (9).

Based on this initial screening pass, top candidates were further validated for broad activity by testing again on the initial screen sites and additionally targeting the K19 and H36 codons in the endogenous CTNNB1 transcript after the first round of selection (FIGS. 37C-37F), and additionally on Gaussia luciferase reporters with G92R, R93K and R93Q catalytic mutations as well as the targeting of the T41 codon in CTNNB1 (FIGS. 38D-38J). Based on activity at all tested sites as measured by either next-generation sequencing and luciferase assays, as well as specificity measured as described, a single top candidate was identified and cloned into the RanCas13b-REPAIR yeast expression construct derived from the previous round of evolution to use as a basis for mutagenesis for the subsequent round.

After Round 1, Applicants identified the E620G mutation and after Round 2, Applicants identified the Q696L mutation. We additionally identified Y505I as a mutation capable of enhancing editing at target sites with a 5′G(FIGS. 38A-38J).

TABLE 32
Accessions of contigs containing Cas13b-torthologs

| | | | | Sample |
|---|---|---|---|---|
| | | | | collection |
| Source | | Ortholog name | | temperature |
| database | Contig accession | (if applicable) | Source habitat/organism | (C.) |
| JGI | Ga0246100_107590 | | Groundwater | |
| JG | Ga0265293_10004442 | | Landfill leachate | |
| JGI | Ga0315543_1000530 | | Salt marsh sediment | |
| JGI | Ga0209381_1018281 | Cas13b-t5 | Hot spring sediment | 64.7 |
| JGI | Ga0310137_000061 | | Fracking water | |
| JGI | Ga0208824_1000897 | | Anaerobic digester sludge | |
| JGI | Ga0137489_1004561 | | Basal ice | |
| JGI | Ga0315552_1001799 | | Salt marsh sediment | |
| JGI | Ga0180434_10014215 | Cas13b-t4 | Hypersaline lake sediment | |
| JGI | Ga0315532_1010951 | | Salt marsh sediment | |
| JGI | Ga0307431_1000754 | | Salt marsh sediment | |
| JGI | Ga0315541_1003536 | | Salt marsh sediment | |
| JGI | Ga0315296_10033793 | | Freshwater lake sediment | |
| JGI | Ga0307443_1009138 | | Salt marsh sediment | |
| JGI | Ga0315532_1006943 | | Salt marsh sediment | |
| JGI | Ga0315554_1005387 | | Salt marsh sediment | |
| NCBI WGS | QNBS01000103.1 | Cas13b-t3 | Planctomycetes bacterium | |
| | | | isolate B28_G16 (marine | |
| | | | sediment) | |
| JGI | Ga0315285_10018775 | | Freshwater lake sediment | |
| JGI | Ga0315294_10038294 | | Freshwater lake sediment | |
| JGI | Ga0315533_1000464 | | Salt marsh sediment | |
| JGI | Ga0209427_10000033 | Cas13b-t2 | Marine sediment | |
| JGI | Ga0114919_10002421 | Cas13b-t1 | Atlantic deep subsurface | |

JGI: Joint Genome Institute
NCBI WGS: National Center for Biotechnology Information Whole Genome Shotgun

TABLE 33
Direct repeat sequences of Cas13 orthologs used in this example

| Organism | Abbreviation key | DR sequence |
|---|---|---|
| Riemerella anatipestifer | Ran | GTTGGGACTGCTCTCACTTTGAAGGGTATTCACAA |
| | | C |
| | | (SEQ ID NO: 5861) |
| Prevotella sp. P5-125 | Psp | GTTGTGGAAGGTCCAGTTTTGAGGGGCTATTACAA |
| | | C |
| | | (SEQ ID NO: 5862) |
| | b-t1 | GCTGTAATCACCCCACAAATCGGAGGCTTCTTCAG |
| | | C |
| | | (SEQ ID NO: 5863) |
| | b-t2 | GCTGTAATCACCCCACAAATCGGGGGCTTCTCCAG |
| | | C |
| | | (SEQ ID NO: 5864) |
| | b-t3 | GCTGTAATCACCCCACAAATCGGGGGCTGCTCCAG |
| | | C |
| | | (SEQ ID NO: 5865) |
| | b-t4 | GCTGTTACTTCCCCACAAATTGAGGCCCATCACAG |
| | | C |
| | | (SEQ ID NO: 5866) |
| | b-t5 | GCTGTGATTACCCTGCAAATCGAGGGCTGCTCCAG |
| | | c |
| | | (SEQ ID NO: 5867) |

TABLE 34
Cas13 orthologs used in this example

| Abbreviation | |
|---|---|
| key | Protein sequence |
| Ran | MEKPLLPNVYTLKHKFFWGAFLNIARHNAFITICHINEQLGLKTPSNDDKIVDVVCETWNNIL |
| | NNDHDLLKKSQLTELILKHFPFLTAMCYHPPKKEGKKKGHQKEQQKEKESEAQSQAEALNP |
| | SKLIEALEILVNQLHSLRNYYSHYKHKKPDAEKDIFKHLYKAFDASLRMVKEDYKAHFTVN |
| | LTRDFAHLNRKGKNKQDNPDFNRYRFEKDGFFTESGLLFFTNLFLDKRDAYWMLKKVSGF |
| | KASHKQREKMTTEVFCRSRILLPKLRLESRYDHNQMLLDMLSELSRCPKLLYEKLSEENKKH |
| | FQVEADGFLDEIEEEQNPFKDTLIRHQDRFPYFALRYLDLNESFKSIRFQVDLGTYHYCIYDK |
| | KIGDEQEKRHLTRTLLSFGRLQDFTEINRPQEWKALTKDLDYKETSNQPFISKTTPHYHITDN |
| | KIGFRLGTSKELYPSLEIKDGANRIAKYPYNSGFVAHAFISVHELLPLMFYQHLTGKSEDLLK |
| | ETVRHIQRIYKDFEEERINTIEDLEKANQGRLPLGAFPKQMLGLLQNKQPDLSEKAKIKIEKLI |
| | AETKLLSHRLNTKLKSSPKLGKRREKLIKTGVLADWLVKDFMRFQPVAYDAQNQPIKSSKA |
| | NSTEFWFIRRALALYGGEKNRLEGYFKQTNLIGNTNPHPFLNKFNWKACRNLVDFYQQYLE |
| | QREKFLEAIKNQPWEPYQYCLLLKIPKENRKNLVKGWEQGGISLPRGLFTEAIRETLSEDLML |
| | SKPIRKEIKKHGRVGFISRAITLYFKEKYQDKHQSFYNLSYKLEAKAPLLKREEHYEYWQQN |
| | KPQSPTESQRLELHTSDRWKDYLLYKRWQHLEKKLRLYRNQDVMLWLMTLELTKNHFKEL |
| | NLNYHQLKLENLAVNVQEADAKLNPLNQTLPMVLPVKVYPATAFGEVQYHKTPIRTVYIRE |
| | EHTKALKMGNFKALVKDRRLNGLFSFIKEENDTQKHPISQLRLRRELEIYQSLRVDAFKETLS |
| | LEEKLLNKHTSLSSLENEFRALLEEWKKEYAASSMVTDEHIAFIASVRNAFCHNQYPFYKEA |
| | LHAPIPLFTVAQPTTEEKDGLGIAEALLKVLREYCEIVKSQI (SEQ ID NO: 5868) |
| Psp | MNIPALVENQKKYFGTYSVMAMLNAQTVLDHIQKVADIEGEQNENNENLWFHPVMSHLYN |
| | AKNGYDKQPEKTMFIIERLQSYFPFLKIMAENQREYSNGKYKQNRVEVNSNDIFEVLKRAFG |
| | VLKMYRDLTNHYKTYEEKLNDGCEFLTSTEQPLSGMINNYYTVALRNMNERYGYKTEDLA |
| | FIQDKRFKFVKDAYGKKKSQVNTGFFLSLQDYNGDTQKKLHLSGVGIALLICLFLDKQYINIF |
| | LSRLPIFSSYNAQSEERRIIIRSFGINSIKLPKDRIHSEKSNKSVAMDMLNEVKRCPDELFTTLS |
| | AEKQSRFRIISDDHNEVLMKRSSDRFVPLLLQYIDYGKLFDHIRFHVNMGKLRYLLKADKTC |
| | IDGQTRVRVIEQPLNGFGRLEEAETMRKQENGTFGNSGIRIRDFENMKRDDANPANYPYIVD |
| | TYTHYILENNKVEMFINDKEDSAPLLPVIEDDRYVVKTIPSCRMSTLEIPAMAFHMFLFGSKK |
| | TEKLIVDVHNRYKRLFQAMQKEEVTAENIASFGIAESDLPQKILDLISGNAHGKDVDAFIRLT |
| | VDDMLTDTERRIKRFKDDRKSIRSADNKMGKRGFKQISTGKLADFLAKDIVLFQPSVNDGEN |
| | KITGLNYRIMQSAIAVYDSGDDYEAKQQFKLMFEKARLIGKGTTEPHPFLYKVFARSIPANA |
| | VEFYERYLIERKFYLTGLSNEIKKGNRVDVPFIRRDQNKWKTPAMKTLGRIYSEDLPVELPR |
| | QMFDNEIKSHLKSLPQMEGIDFNNANVTYLIAEYMKRVLDDDFQTFYQWNRNYRYMDML |
| | KGEYDRKGSLQHCFTSVEEREGLWKERASRTERYRKQASNKIRSNRQMRNASSEEIETILDK |
| | RLSNSRNEYQKSEKVIRRYRVQDALLFLLAKKTLTELADFDGERFKLKEIMPDAEKGILSEIM |
| | PMSFTFEKGGKKYTITSEGMKLKNYGDFFVLASDKRIGNLLELVGSDIVSKEDIMEEFNKYD |
| | QCRPEISSIVFNLEKWAFDTYPELSARVDREEKVDFKSILKILLNNKNINKEQSDILRKIRNAF |
| | DHNNYPDKGVVEIKALPEIAMSIKKAFGEYAIMK (SEQ ID NO: 5869) |
| b-t1 | MEFENIKKTSNKEVYSIEQYEGEKKWCFAIVLNRAQTNLEENPKLFEQTLTRFEKIMKQDWFN |
| | EETKKLIYEKEEENKVKEEIQIAASERLKNLANYFSAYLHAPDCLIFNRNDTIRIIMEKAYEKSR |
| | FEAKKKQQEDISIEFPELFEEEDKITSAGVVFFVSFFIERRFLNRLMGYVQGFRKTEGEYNITRQ |
| | VFSKYCLKDSYSVQAQDHDAVMFRDILGYLSRVPTEIYQHIKLTRKRSQDQLSERKTDKFILF |
| | ALKYLEDYGLKDLADYTACFARSKIKRENEDTKETDGNKHKFHREKPVVEIHFDKEKQDQFY |
| | IKRNNVILKAQKKGGQSNVFRMGVYELKYLVLLSLLGKAEEAIQRIDRYISSLKKQLPYLDKIS |
| | NEEIQKSINFLPRFVRSRLGLLQVDDEKRLKTRLEYVKAKWTDKKEGSRKLELHRKGRDILRY |
| | INERCDRPLSRKEYNNILKFIVNKDFAGFYNELEELKRTRRLDKNIIQKLSGHTTLNALHERVC |
| | DLVLQELGSLQSENLKEYIGLIPKEEKEVTFREKVDRILEQPVVYKGFLRYEFFKEDKKSFARL |
| | VEEAIKTKWSDFDIPLGEEYYNIPSLDRFDRTNKKLYETLAMDRLCLMMARQYYLRLNEKLA |
| | EKAQHIYWKKEDGREVIIFKFQNPKEQKKSFSIRFSILDYTKMYVMDDPEFLSRLWEYFIPKEA |
| | KEIDYHKHYARAFDKYTNLQKEGIDAILKLEGRIIERRKIKPAKNYIEFQEIMNRSGYNNDQQV |
| | ALKRVANALLAYNLNFEREHLKRFYGVVKREGIEKKWSLIV (SEQ ID NO: 5870) |
| b-t2 | MQVENIKKGSSQGMYSIEQYEGAKKWCFAIVLNRAQTNLQGNPKLFEETLTRFERIRKEDWF |
| | DQETKKLIYAKQEQNEVEEEIQKAADEKLRDLRNYFSHYFHTPDCLIFTQNDPVRIIMEKAYE |
| | KARFEQAKKEQEDISIEFGELFEENGRITSAGVVFFASFFAERRFLNRLMGYVQGFTRTEGEYK |
| | ITRDVFSTYCLRDSYSVKTPDHDAVMFRDILGYLSRVPSESYQRIKESQMRSETQLSERKTDKF |
| | ILFALNYLEDYGLEDLADYTACFARTRIKREQDENTDGKEQKPHRKKPRVEIHFERAEGDPFYI |
| | KHNNVILRTQKKGAQTYIFRMGVYELKYLVLLSLLGKGAEAVKRIDRYVHSLRNQLPHIEKK |
| | STEEIEGYVRFLPRFVRSHLGLLGVDDEKKIKARVDYVKAKWLEKKEKSRELQLHRKGRDILR |
| | YINERCERPLNIDEYNRILELLVTKHLDGFYRELEELKKTRRIDKNIVCNLSRHKSVNALHEKV |
| | CDLVVQELESLGREELKEYVGLIPKEEKEVSFEEKTDRVVKQPVIYKGFLRNEFFRESRKSFAR |
| | LVEEAVREKGEVYDVPLGGEYYEIVSLDTFDKDNKRLYETLAMDRLLLMIARQYHLSLNKEL |
| | AKRAQQIEWKKEDGEEVIIFTLKNPAQPEQSCSVRFSLRDYTKLYVMDDAEFLARLCDYFLPK |
| | DEEQIDYHRLYTQGMNRYTNLQREGIEAILELEKKTIGPEQPRPPKNYIPFSEIMDKSAYNEDD |
| | QKALRRVRNALLHHNLNFARADFKRFCGIMKREGIEKRWSLAV (SEQ ID NO: 5871) |
| b-t3 | MAQVSKQTSKKRELSIDEYQGARKWCFTIAFNKALVNRDKNDGLFVESLLRHEKYSKHDWY |
| | DEDTRALIKCSTQAANAKAEALANYFSAYRHSPGCLTFTAEDELRTIMERAYERAIFECRRRE |
| | TEVIIEFPSLFEGDRITTAGVVFFVSFFVERRVLDRLYGAVSGLKKNEGQYKLTRKALSMYCLK |
| | DSRFTKAWDKRVLLFRDILAQLGRIPAEAYEYYHGEQGDKKRANDNEGTNPKRHKDKFIEFA |
| | LHYLEAQHSEICFGRRHIVREEAGAGDEHKKHRTKGKVVVDFSKKDEDQSYYISKNNVIVRID |
| | KNAGPRSYRMGLNELKYLVLLSLQGKGDDAIAKLYRYRQHVENILDVVKVTDKDNHVFLPR |
| | FVLEQHGIGRKAFKQRIDGRVKHVRGVWEKKKAATNEMTLHEKARDILQYVNENCTRSFNP |
| | GEYNRLLVCLVGKDVENFQAGLKRLQLAERIDGRVYSIFAQTSTINEMHQVVCDQILNRLCRI |
| | GDQKLYDYVGLGKKDEIDYKQKVAWFKEHISIRRGFLRKKFWYDSKKGFAKLVEEHLESGG |
| | GQRDVGLDKKYYHIDAIGRFEGANPALYETLARDRLCLMMAQYFLGSVRKELGNKIVWSND |
| | SIELPVEGSVGNEKSIVFSVSDYGKLYVLDDAEFLGRICEYFMPHEKGKIRYHTVYEKGFRAY |
| | NDLQKKCVEAVLAFEEKVVKAKKMSEKEGAHYIDFREILAQTMCKEAEKTAVNKVARAFFA |
| | HHLKFVIDEFGLFSDVMKKYGIEKEWKFPVK (SEQ ID NO: 5872) |
| b-t4 | MNIIKLKKEEAAFYFNQTILNLSGLDEIIEKQIPHIISNKENAKKVIDKIFNNRLLLKSVENYIYNF |
| | KDVAKNARTEIEAILLKLVELRNFYSHYVHNDTVKILSNGEKPILEKYYQIAIEATGSKNVKLV |
| | IIENNNCLTDSGVLFLLCMFLKKSQANKLISSVSGFKRNDKEGQPRRNLFTYYSVREGYKVVP |
| | DMQKHFLLFALVNHLSEQDDHIEKQQQSDELGKGLFFHRIASTFLNESGIFNKMQFYTYQSNR |
| | LKEKRGELKHEKDTFTWIEPFQGNSYFTLNGHKGVISEDQLKELCYTILIEKQNVDSLEGKIIQF |
| | LKKFQNVSSKQQVDEDELLKREYFPANYFGRAGTGTLKEKILNRLDKRMDPTSKVTDKAYD |
| | KMIEVMEFINMCLPSDEKLRQKDYRRYLKMVRFWNKEKHNIKREFDSKKWTRFLPTELWNK |
| | RNLEEAYQLARKENKKKLEDMRNQVRSLKENDLEKYQQINYVNDLENLRLLSQELGVKWQE |
| | KDWVEYSGQIKKQISDNQKLTIMKQRITAELKKMHGIENLNLRISIDTNKSRQTVMNRIALPK |
| | GFVKNHIQQNSSEKISKRIREDYCKIELSGKYEELSRQFFDKKNFDKMTLINGLCEKNKLIAFM |
| | VIYLLERLGFELKEKTKLGELKQTRMTYKISDKVKEDIPLSYYPKLVYAMNRKYVDNIDSYAF |
| | AAYESKKAILDKVDIIEKQRMEFIKQVLCFEEYIFENRIIEKSKFNDEETHISFTQIHDELIKKGR |
| | DTEKLSKLKHARNKALHGEIPDGTSFEKAKLLINEIKK (SEQ ID NO: 5873) |
| b-t5 | MGIDYSLTSDCYRGINKSCFAVALNIAYDNCDHKGCRTLLSEVLRSKGGISDEQIKSQVVDGIQ |
| | KRLKDIRNYFSHYYHAEDCLRFGDQDAVKVFLEEIYKNAESKTVGATKESDYKGVVPPLFEL |
| | HNGTYMITAAGVIFLASFFCHRSNVYRMLGAVKGFKHTGKEQLSDGQKRDYGFTRRLLAYY |
| | ALRDSYSVGAEDKTRCFREILSYLSRVPQLAVDWLNEQQLLTPEEKEAFLNQPAEDEGGDISD |
| | SSSSDKNKKSKEKRRSLRRDEKFILFAIQFIEGWAAEQGLDVTFARYQKTVEKAENKNQDGKQ |
| | ARAVQLKYRNQGLNPDFNNEWMYYIQNEHAIIQIKLNNKKAVAARISENELKYLVLLIFEEKG |
| | NDAVQKLNCYIYSMSQKIEGEWKHRPEDERWMPSFTKRADRTVTPEAVQSRLSYIRKQLQETI |
| | EKIGQEEPRNNKWLIYKGKKISMILKFISDSIRDIQRRPNVKQYHILRDALQRLDFDGFYKELQ |
| | NYVNDGRIAVSLYDQIKGVNDISGLCKKVCELTLERLAGLEAKNGSELRRYIGLEAQEKHPKY |
| | GEWNTLQEKAKRFLESQFSIGKNFLRKMFYGDCCQKRCFDEEKGYNTQAKERKSLYSIVKEK |
| | LKDIKPIHDDRWYLIDRNPKNYDNKHSRIIRQMCNTYIQDVLCMKMAMWHYEKLISATEFRN |
| | KLEWNCIGQGNMGYERYSLWYKTGCGVVIQFTPADFLRLDIIEKPAMIENICQCFVLGNKKLN |
| | SGAEKKITWDKFNKDGIAKYRKRQAEAVRAIFAFEEGLKIQEDKWSHERYFPFCNILDEAVKQ |
| | GKIKDTGKDKEALNRGRNDFFHEEFKSTEDQQAIFQKYFPIVERKDDTKKRRDKKQK |
| | (SEQ ID NO: 5874) |

TABLE 35
Primers for cloning plasmids used in PFS screen

| Name | Sequence |
|---|---|
| Cas13b-t1_gene_F | TGCCGGGCCTCTTGCGGGATTTTACACTTTATGCTTCCGGCTCGTATGTTA |
| | GGAGGTCTTTATCATGGAATTCGAGAACATCAA (SEQ ID NO: 5875) |
| Cas13b-t1_gene_R | ATGCTGTCGGAATGGACGATTCACACGATCAGGGACCATT (SEQ ID NO: |
| | 5876) |
| Cas13b-t2_gene_F | TGCCGGGCCTCTTGCGGGATTTTACACTTTATGCTTCCGGCTCGTATGTTA |
| | GGAGGTCTTTATCATGCAGGTCGAGAACATCAA (SEQ ID NO: 5877) |
| Cas13b-t2_gene_R | ATGCTGTCGGAATGGACGATTCACACAGCCAGGGACCATC |
| | (SEQ ID NO: 5878) |
| Cas13b-t3_gene_F | TGCCGGGCCTCTTGCGGGATTTTACACTTTATGCTTCCGGCTCGTATGTTA |
| | GGAGGTCTTTATCATGGCCCAGGTGTCCAAGCA (SEQ ID NO: 5879) |
| Cas13b-t3_gene_R | ATGCTGTCGGAATGGACGATTCACTTCACGGGGAACTTCC |
| | (SEQ ID NO: 5880) |
| Cas13b-t4_gene_F | TGCCGGGCCTCTTGCGGGATTTTACACTTTATGCTTCCGGCTCGTATGTTA |
| | GGAGGTCTTTATCATGAACATCATCAAGCTGAA (SEQ ID NO: 5881) |
| Cas13b-t4_gene_R | ATGCTGTCGGAATGGACGATTTACTTCTTAATCTCATTGA |
| | (SEQ ID NO: 5882) |
| Cas13b-t5_gene_F | TGCCGGGCCTCTTGCGGGATTTTACACTTTATGCTTCCGGCTCGTATGTTA |
| | GGAGGTCTTTATCATGGGCATCGATTACAGCCT (SEQ ID NO: 5883) |
| Cas13b-t5_gene_R | ATGCTGTCGGAATGGACGATTTACTTCTGCTTCTTGTCTC |
| | (SEQ ID NO: 5884) |
| crRNA_expression_ | TGCCGGGCCTCTTGCGGGATATCTTGACAGCTAGCTCAGTCCT |
| bac_F | (SEQ ID NO: 5885) |
| Cas13b-t1_crRNA_R | GCGTCCGGCGTAGAGGATCCGCTGTAATCACCCCACAAAT |
| | (SEQ ID NO: 5886) |
| Cas13b-t2_crRNA_R | GCGTCCGGCGTAGAGGATCCGCTGTAATCACCCCACAAAT |
| | (SEQ ID NO: 5887) |
| Cas13b-t3_crRNA_R | GCGTCCGGCGTAGAGGATCCGCTGTAATCACCCCACAAAT |
| | (SEQ ID NO: 5888) |
| Cas13b-t4_crRNA_R | GCGTCCGGCGTAGAGGATCCGCTGTTACTTCCCCACAAAT |
| | (SEQ ID NO: 5889) |
| Cas13b-t5_crRNA_R | GCGTCCGGCGTAGAGGATCCGCTGTGATTACCCTGCAAAT |
| | (SEQ ID NO: 5890) |

TABLE 11
Primers for cloning mammalian expression plasmids. Mutations
introduced by PCR are shown in lower case.

| Name | Sequence |
|---|---|
| crRNA_expression_ | GACCGAGCGCAGCGAGTCAGTGAGCGAGGA (SEQ ID NO: 5891) |
| mammalian_F | |
| Cas13b- | AACGACGGCCAGTGAATTCGAGCTCGGTACCAAAAAAGCTGTAATCACCCCAC |
| t1_crRNA_mammalian_ | AAATCGGAGGCTTCTTCAGCTTGTCTTCGTCCCAGGAAGACATGGTGTTTCGTC |
| R | CTTTCCACAAGATATATAAA (SEQ ID NO: 5892) |
| Cas13b- | AACGACGGCCAGTGAATTCGAGCTCGGTACCAAAAAAGCTGTAATCACCCCAC |
| t3_crRNA_mammalian_ | AAATCGGGGGCTGCTCCAGCTTGTCTTCGTCCCAGGAAGACATGGTGTTTCGT |
| R | CCTTTCCACAAGATATATAAA (SEQ ID NO: 5893) |
| Cas13b- | AACGACGGCCAGTGAATTCGAGCTCGGTACCAAAAAAGCTGTGATTACCCTGC |
| t5_crRNA_mammalian_ | AAATCGAGGGCTGCTCCAGCTTGTCTTCGTCCCAGGAAGACATGGTGTTTCGT |
| R | CCTTTCCACAAGATATATAAA (SEQ ID NO: 5894) |
| Cas13b- | GAGACCCAAGCTGGCTAGCGTTTAAACTTAAGCTTGCCACCATGGGATCCCTT |
| t1_gene_mammalian_ | CAACTGCCTCCACTTGAAAGACTGACACTGGGATCCGAATTCGAGAACATCAA |
| F | GAAAA (SEQ ID NO: 5895) |
| Cas13b- | CAGGTAGgcGCTGAAGTAGTTTgcCAGGTTC (SEQ ID NO: 5896) |
| t1_HEPN1_mut_R | |
| Cas13b- | GAACCTGgcAAACTACTTCAGCgcCTACCTG (SEQ ID NO: 5897) |
| t1_HEPN1_mut_F | |
| Cas13b- | GTTGTAGgcCAGCAGGGCGTTCgcCACTCTC (SEQ ID NO: 5898) |
| t1_HEPN2_mut_R | |
| Cas13b- | GAGAGTGgcGAACGCCCTGCTGgcCTACAAC (SEQ ID NO: 5899) |
| t1_HEPN2_mut_F | |
| Cas13b- | ACTACCGCCTGACCCTCCCACGATCAGGGACCATTTTTTCTCG |
| t1_forREPAIR_R | (SEQ ID NO: 5900) |
| Cas13b- | GAGACCCAAGCTGGCTAGCGTTTAAACTTAAGCTTGCCACCATGGGATCCCTT |
| t3_gene_mammalian_ | CAACTGCCTCCACTTGAAAGACTGACACTGGGATCCGCCCAGGTGTCCAAGCA |
| F | GACCA (SEQ ID NO: 5901) |
| Cas13b- | TCTGTAGgcGCTGAAGTAGTTTgcCAGAGCC (SEQ ID NO: 5902) |
| t3_HEPN1_mut_R | |
| Cas13b- | GGCTCTGgcAAACTACTTCAGCgcCTACAGA (SEQ ID NO: 5903) |
| t3_HEPN1_mut_F | |
| Cas13b- | GTGGTGGgcAAAGAAGGCTCTCgcCACTTTG (SEQ ID NO: 5904) |
| t3_HEPN2_mut_R | |
| Cas13b- | CAAAGTGgcGAGAGCCTTCTTTgcCCACCAC (SEQ ID NO: 5905) |
| t3_HEPN2_mut_F | |
| Cas13b- | ACTACCGCCTGACCCTCCCTTCACGGGGAACTTCCATTCTTTC |
| 13_forREPAIR_R | (SEQ ID NO: 5906) |
| Cas13b- | GAGACCCAAGCTGGCTAGCGTTTAAACTTAAGCTTGCCACCATGGGATCCCTT |
| t5_gene_mammalian_ | CAACTGCCTCCACTTGAAAGACTGACACTGGGATCCATGGGCATCGATTACAG |
| F | CCTGACCA (SEQ ID NO: 5907) |
| ADAR2_F | GGAGGGTCAGGCGGTAGTCAGCTGCATTTA (SEQ ID NO: 5908) |
| pcDNA_expression_ | GGGTTTAAACGGGCCCTCTAGACTC (SEQ ID NO: 5909) |
| R | |

TABLE 37
Primers for cloning yeast constructs used in this example

| Name | Sequence |
|---|---|
| ADAR_mut_library_F | CCAGATCGGGGGTTCCGGCGGGTCC (SEQ ID NO: 5910) |
| ADAR_mut_library_R | TATTTAATAATAAAAATCATAAATCATAAGAAATTCGCCACGTGAGTCTA |
| | GGATCCTCA (SEQ ID NO: 5911) |
| URA3_F | ACTCACTATAGGGAATATTAAGCTTTTCAATTCATCATTTTTTTT |
| | (SEQ ID NO: 5912) |
| URA3_R | TTAGTTTTGCTGGCCGCATC (SEQ ID NO: 5913) |
| URA3_TAG_R | AATGTCTGCCTATTCTGCTAT (SEQ ID NO: 5914) |
| URA3_TAG_F | ATAGCAGAATAGGCAGACATT (SEQ ID NO: 5915) |
| ADE2_F | GGGCGCGTGGGGATGATCCATTCTTGAATAATACATAACT |
| | (SEQ ID NO: 5916) |
| ADE2_R | AAACAACAAAAGGATACTAGTCGCTATCCTCGGTTCTGCAT |
| | (SEQ ID NO: 5917) |
| ADE2_TGA_R | TTAGTAAATGGTGCTCATTTTTCGGCGTACA (SEQ ID NO: 5918) |
| ADE2_TGA_F | TGTACGCCGAAAAATGAGCACCATTTACTAA (SEQ ID NO: 5919) |
| LEU2_F | AACTGTGGGAATACTCAGGTATCGT (SEQ ID NO: 5920) |
| LEU2_R | TTAAGCAAGGATTTTCTTAACTTCTTCGGC (SEQ ID NO: 5921) |
| ADAR2_yeast_F | CCAGATCGGGGGTTCCGGCGGGTCC (SEQ ID NO: 5922) |
| ADAR2_yeast_R | GAACAAAAGCTGGAGCTCCACCG (SEQ ID NO: 5923) |
| E620G_yeast_R | GACGCCCTGCCTAACcCATCTTTGCCGGTCG (SEQ ID NO: 5924) |
| E620G_yeast_F | CGACCGGCAAAGATGgGTTAGGCAGGGCGTC (SEQ ID NO: 5925) |
| ADE2 targeting spacer | CGTCAATGGTGCcCATTTTTCGGCGTACAAAGGA (SEQ ID NO: 5926) |
| (22 bp mismatch) | |

TABLE 38
Next-generation sequencing library preparation first round PCR primers
for PFS screen

| Name | Sequence |
|---|---|
| PFS_NGS_F1 | CTTTCCCTACACGACGCTCTTCCGATCTCGCTAGCTCAGTCCTAGGTA |
| | TAATGCTAGC (SEQ ID NO: 5927) |
| PFS_NGS_F1 | CTTTCCCTACACGACGCTCTTCCGATCTACGCTAGCTCAGTCCTAGGT |
| | ATAATGCTAGC (SEQ ID NO: 5928) |
| PFS_NGS_F1 | CTTTCCCTACACGACGCTCTTCCGATCTGACGCTAGCTCAGTCCTAGG |
| | TATAATGCTAGC (SEQ ID NO: 5929) |
| PFS_NGS_F1 | CTTTCCCTACACGACGCTCTTCCGATCTTGACGCTAGCTCAGTCCTAG |
| | GTATAATGCTAGC (SEQ ID NO: 5930) |
| Cas13b-t1_PFS_NGS_R | GACTGGAGTTCAGACGTGTGCTCTTCCGATCTCCACAAATCGGAGGC |
| | TTCTTCAGC (SEQ ID NO: 5931) |
| Cas13b-t2_PFS_NGS_R | GACTGGAGTTCAGACGTGTGCTCTTCCGATCTCAAATCGGGGGCTTCT |
| | CCAGC (SEQ ID NO: 5932) |
| Cas13b-t3_PFS_NGS_R | GACTGGAGTTCAGACGTGTGCTCTTCCGATCTATCGGGGGCTGCTCCA |
| | GC (SEQ ID NO: 5933) |
| Cas13b-t4_PFS_NGS_R | GACTGGAGTTCAGACGTGTGCTCTTCCGATCTCCACAAATTGAGGCC |
| | CATCACAGC (SEQ ID NO: 5934) |
| Cas13b-t5_PFS_NGS_R | GACTGGAGTTCAGACGTGTGCTCTTCCGATCTCAAATCGAGGGCTGC |
| | TCCAGC (SEQ ID NO: 5935) |

TABLE 39
gRNA spacer sequences for Gaussia luciferase knockdown in
HEK293FT cells. Relative expression is as measured by depletion of luciferase activity
compared to a GFP control.

| | | Bt1 relative | Bt3 relative | Bt5 relative |
|---|---|---|---|---|
| | | expression | expression | expression |
| Name | Spacer sequence | (FIG. 1g) | (FIG. 1g) | (FIG. 1g) |
| Gaussia luciferase | TTTGTCGCCTTCGTAGGTGTG | 0.55062748 | 0.38468809 | 0.3397597 |
| spacer 1 | GCAGCGTCC | | | |
| | (SEQ ID NO: 5936) | | | |
| Gaussia luciferase | CCAGGAATCTCAGGAATGTC | 0.5538685 | 0.35529333 | 0.44593268 |
| spacer 2 | GACGATCGCC | | | |
| | (SEQ ID NO: 5937) | | | |
| Gaussia luciferase | GTCGACGATCGCCTCGCCTAT | 0.38844716 | 0.29975324 | 0.45171626 |
| spacer 3 | GCCGCCCTG | | | |
| | (SEQ ID NO: 5938) | | | |
| Gaussia luciferase | CGATGAACTGCTCCATGGGC | 0.74248373 | 0.7076885 | 0.62729858 |
| spacer 4 | TCCAAGTCCT | | | |
| | (SEQ ID NO: 5939) | | | |
| Gaussia luciferase | TCGCGAAGTTGCTGGCCACG | 0.48985892 | 0.56514571 | 0.34100497 |
| spacer 5 | GCCACGATGT | | | |
| | (SEQ ID NO: 5940) | | | |
| Gaussia luciferase | CAGCCCCTGGTGCAGCCAGC | 0.73143084 | 0.45223832 | 0.59533757 |
| spacer 6 | TTTCCGGGCA | | | |
| | (SEQ ID NO: 5941) | | | |
| Gaussia luciferase | GGCCCCCTTGATCTTGTCCAC | 0.51439053 | 0.21018064 | 0.37180078 |
| spacer 7 | CTGGCCCTG | | | |
| | (SEQ ID NO: 5942) | | | |
| Gaussia luciferase | GATGTGGGACAGGCAGATCA | 0.65183105 | 0.46064806 | 0.51302536 |
| spacer 8 | GACAGCCCCT | | | |
| | (SEQ ID NO: 5943) | | | |
| Gaussia luciferase | CGTTGCGGCAGCCACTTCTTG | 0.42079237 | 0.26868947 | 0.35726081 |
| spacer 9 | AGCAGGTCA | | | |
| | (SEQ ID NO: 5944) | | | |
| Gaussia luciferase | TGTCGACGATCGCCTCGCCTA | 0.63580411 | 0.30643568 | 0.36228356 |
| spacer 10 | TGCCGCCCT | | | |
| | (SEQ ID NO: 5945) | | | |
| Gaussia luciferase | CTCGGCCACAGCGATGCAGA | 0.57120708 | 0.42967329 | 0.31493639 |
| spacer 11 | TCAGGGCAAA | | | |
| | (SEQ ID NO: 5946) | | | |
| Gaussia luciferase | CCTTGAACCCAGGAATCTCA | 0.58010478 | 0.27618297 | 0.4351957 |
| spacer 12 | GGAATGTCGA | | | |
| | (SEQ ID NO: 5947) | | | |
| Gaussia luciferase | CCGGGCATTGGCTTCCATCTC | 0.57770913 | 0.33286417 | 0.30132433 |
| spacer 13 | TTTGAGCAC | | | |
| | (SEQ ID NO: 5948) | | | |
| Gaussia luciferase | ACAGGCAGATCAGACAGCCC | 0.77305496 | 0.54830739 | 0.69564972 |
| spacer 14 | CTGGTGCAGC | | | |
| | (SEQ ID NO: 5949) | | | |
| Gaussia luciferase | GTCACCACCGGCCCCCTTGAT | 0.74591779 | 0.65311879 | 0.65571849 |
| spacer 15 | CTTGTCCAC | | | |
| | (SEQ ID NO: 5950) | | | |
| Gaussia luciferase | CTTGATGTGGGACAGGCAGA | 0.62758078 | 0.47392894 | 0.43519874 |
| spacer 16 | TCAGACAGCC | | | |
| | (SEQ ID NO: 5951) | | | |
| Gaussia luciferase | CTGGCCCTGGATCTTGCTGGC | 0.4753283 | 0.18260215 | 0.32822212 |
| spacer 17 | AAAGGTCGC | | | |
| | (SEQ ID NO: 5952) | | | |
| Gaussia luciferase | GGGCTCCAAGTCCTTGAACC | 0.60092434 | 0.3810874 | 0.42402921 |
| spacer 18 | CAGGAATCTC | | | |
| | (SEQ ID NO: 5953) | | | |
| Gaussia luciferase | ATGAACTGCTCCATGGGCTCC | 0.5442858 | 0.28338889 | 0.55534371 |
| spacer 19 | AAGTCCTTG | | | |
| | (SEQ ID NO: 5954) | | | |
| Gaussia luciferase | TCGAGATCCGTGGTCGCGAA | 0.48568996 | 0.35394464 | 0.36567816 |
| spacer 20 | GTTGCTGGCC | | | |
| | (SEQ ID NO: 5955) | | | |
| Non-targeting spacer 1 | CCTCTGAAACGATGGTGCAT | 0.71872993 | 0.76013732 | 0.65806897 |
| | GGTAGTGACC | | | |
| | (SEQ ID NO: 5956) | | | |
| Non-targeting spacer 2 | CCTACAGGTTCTGAGTGGGT | 0.85589957 | 0.70637393 | 0.70777278 |
| | GCACGGCCGT | | | |
| | (SEQ ID NO: 5957) | | | |
| Non-targeting spacer 3 | GAAAATGGCCTATACCTTAG | 0.78372124 | 0.72463671 | 0.71052405 |
| | GGTTCGCGCG | | | |
| | (SEQ ID NO: 5958) | | | |
| Non-targeting spacer 4 | GTAATGCCTGGCTTGTCGACG | 0.7999484 | 0.72841838 | 0.72973389 |
| | CATAGTCTG | | | |
| | (SEQ ID NO: 5959) | | | |
| Gaussia luciferase | GGGCATTGGCTTCCATCTCTT | | | |
| guide 1 (Supplementary | TGAGCACCT | | | |
| FIG. 2) | (SEQ ID NO: 5960) | | | |
| Gaussia luciferase | GGAATGTCGACGATCGCCTC | | | |
| guide 2 (Supplementary | GCCTATGCCG | | | |
| FIG. 2) | (SEQ ID NO: 5961) | | | |

TABLE 40
gRNA spacer sequences for endogenous transcript knockdown in
HEK293FT cells. Relative expression is as measured by qPCR as compared to GFP control.

| | | Bt1 relative | Bt3 relative |
|---|---|---|---|
| | | expression | expression |
| Name | Spacer sequence | (FIG. 1h) | (FIG. 1h) |
| CXCR4 spacer 1 | AGAGGTTGACTGTGTAGATGACATGGACTG | 0.4891226 | 0.43242082 |
| | (SEQ ID NO: 5962) | | |
| CXCR4 spacer 2 | GACAGGTGCAGCCTGTACTTGTCCGTCATG | 0.52526336 | 0.4274336 |
| | (SEQ ID NO: 5963) | | |
| CXCR4 spacer 3 | AAAGAGGAGGTCGGCCACTGACAGGTGCAG | 0.55379783 | 0.4922889 |
| | (SEQ ID NO: 5964) | | |
| STAT1 spacer 1 | CAGGAAGAAGGACAGATTCCTGGGTTCCGC | 0.1702036 | 0.26428803 |
| | (SEQ ID NO: 5965) | | |
| STAT1 spacer 2 | CCCAACATGTTCAGCTGGTCCACATTGAGA | 0.19275451 | 0.28221787 |
| | (SEQ ID NO: 5966) | | |
| STAT1 spacer 3 | ATTGAGACCTCTTTTGGTGACAGAAGAAAA | 0.32015 | 0.36930278 |
| | (SEQ ID NO: 5967) | | |
| STAT3 spacer 1 | GCAGCTCCTCAGTCACAATCAGGGAAGCAT | 0.54048912 | 0.43548581 |
| | (SEQ ID NO: 5968) | | |
| STAT3 spacer 2 | CGGTCTCAAAGGTGATCAGGTGCAGCTCCT | 0.61276978 | 0.49713932 |
| | (SEQ ID NO: 5969) | | |
| STAT3 spacer 3 | CTCGGTCTCAAAGGTGATCAGGTGCAGCTC | 0.5595753 | 0.4890901 |
| | (SEQ ID NO: 5970) | | |
| HRAS spacer 1 | GCGTGCAGCCAGGTCACACTTGTTCCCCAC | 0.43228711 | 0.40269921 |
| | (SEQ ID NO: 5971) | | |
| HRAS spacer 2 | GAGCCTGCCGAGATTCCACAGTGCGTGCAG | 0.84377946 | 0.35728849 |
| | (SEQ ID NO: 5972) | | |
| HRAS spacer 3 | AGTGCGTGCAGCCAGGTCACACTTGTTCCC | 0.49958394 | 0.47062756 |
| | (SEQ ID NO: 5973) | | |
| PPIB spacer 1 | CTGTCTTGGTGCTCTCCACCTTCCGCACCA | 0.47982775 | 0.42696786 |
| | (SEQ ID NO: 5974) | | |
| PPIB spacer 2 | GGGAGCCGTTGGTGTCTTTGCCTGCGTTGG | 0.33558372 | 0.33160707 |
| | (SEQ ID NO: 5975) | | |
| PPIB spacer 3 | CGTAGATGCTCTTTCCTCCTGTGCCATCTC | 0.36443656 | 0.3902864 |
| | (SEQ ID NO: 5976) | | |

TABLE 41
TaqMan probes used for qPCR

| Gene | TaqMan assay ID |
|---|---|
| CXCR4 | Hs00607978_s1 |
| STAT1 | Hs01013996_m1 |
| STAT3 | Hs00374280_m1 |
| HRAS | Hs00978050_g1 |
| PPIB | Hs00168719_m1 |
| GAPDH | Hs99999905_m1 |

TABLE 42
gRNA spacer sequences for Cypridina luciferase W85X reporter RNA
editing. Mismatch is denoted by lower case.

| | | | Cas13b-t1 | Cas13b-t3 |
|---|---|---|---|---|
| | Mismatch | | normalized RLU | normalized RLU |
| Site | distance | Spacer sequence | (FIG. 2b) | (FIG. 2b) |
| Cypridina | 2 | GAATCTCTTTCCATAGAATGTT | 0.01260676 | 0.00488241 |
| luciferase | | CTAAACcA (SEQ ID NO: 5977) | | |
| X95W | 4 | ATCTCTTTCCATAGAATGTTCT | 0.0085265 | 0.01001933 |
| | | AAACcATC (SEQ ID NO: 5978) | | |
| | 6 | CTCTTTCCATAGAATGTTCTAA | 0.01200875 | 0.00755633 |
| | | ACcATCCT (SEQ ID NO: 5979) | | |
| | 8 | CTTTCCATAGAATGTTCTAAA | 0.05522275 | 0.01003733 |
| | | CcATCCTGC (SEQ ID NO: 5980) | | |
| | 10 | TTCCATAGAATGTTCTAAACCA | 0.079744 | 0.01706967 |
| | | TCCTGCGG (SEQ ID NO: 5981) | | |
| | 12 | CCATAGAATGTTCTAAACcATC | 0.085725 | 0.05851067 |
| | | CTGCGGCC (SEQ ID NO: 5982) | | |
| | 14 | ATAGAATGTTCTAAACCATCCT | 0.14881 | 0.05986233 |
| | | GCGGCCTC (SEQ ID NO: 5983) | | |
| | 16 | AGAATGTTCTAAACcATCCTGC | 0.1930325 | 0.06880567 |
| | | GGCCTCTA (SEQ ID NO: 5984) | | |
| | 18 | AATGTTCTAAACcATCCTGCGG | 0.93053875 | 0.573424 |
| | | CCTCTACT (SEQ ID NO: 5985) | | |
| | 20 | TGTTCTAAACcATCCTGCGGCC | 0.99714725 | 0.69095267 |
| | | TCTACTCT (SEQ ID NO: 5986) | | |
| | 22 | TTCTAAACcATCCTGCGGCCTC | 1.028434 | 0.80134633 |
| | | TACTCTGC (SEQ ID NO: 5987) | | |
| | 24 | CTAAACcATCCTGCGGCCTCTA | 0.49195525 | 0.27232267 |
| | | CTCTGCAT (SEQ ID NO: 5988) | | |
| | 26 | AAACcATCCTGCGGCCTCTACT | 0.35265576 | 0.36477816 |
| | | CTGCATTC (SEQ ID NO: 5989) | | |
| | 28 | ACcATCCTGCGGCCTCTACTCT | 0.59331175 | 0.36037733 |
| | | GCATTCAA (SEQ ID NO: 5990) | | |
| | 30 | cATCCTGCGGCCTCTACTCTGC | 0.01181 | 0.00410067 |
| | | ATTCAATT (SEQ ID NO: 5991) | | |
| Non- | | GTAATGCCTGGCTTGTCGACG | 0.007946 | 0.003682 |
| targeting | | CATAGTCTG (SEQ ID NO: 5992) | | |

TABLE 43
Optimal gRNA spacer sequences for RNA editing of endogenous
transcripts. Mismatch is denoted by lower case.

| | Mismatch | | Editing | Cas13b-t1 editing |
|---|---|---|---|---|
| Site | distance | Spacer sequence | system | rate (FIG. 2c) |
| STAT1 | 22 | TCTTGATAcATCCAGTTCCTT | REPAIR | 0.2551795 |
| Y701C | | TAGGGCCAT | | |
| | | (SEQ ID NO: 5993) | | |
| STAT3 | 20 | GGTCTTCAGGCATGGGGCAG | REPAIR | 0.21902363 |
| Y705C | | CGCTACCTGG | | |
| | | (SEQ ID NO: 5994) | | |
| LATS1 | 22 | TCGGAAGGcAAATTCATAGA | REPAIR | 0.20295815 |
| T1079A | | ATGCATGTTC | | |
| | | (SEQ ID NO: 5995) | | |
| CTNNB1 | 22 | AGCTGTGGcAGTGGCACCAG | REPAIR | 0.39486936 |
| T41A | | AATGGATTCC | | |
| | | (SEQ ID NO: 5996) | | |
| Gaussia | 14 | TTCATCTTGGGCGTGCCATT | RESCUE | 0.47878881 |
| luciferase | | GATGTGGGAC | | |
| C82R | | (SEQ ID NO: 5997) | | |
| CTNNB1 | 22 | GAGCTGTGtTAGTGGCACCA | RESCUE | 0.03803724 |
| T41I | | GAATGGATTC | | |
| | | (SEQ ID NO: 5998) | | |
| KRAS | 20 | GATCATATTCtTCCACAAAA | RESCUE | 0.06281841 |
| D30D | | TGATTCTGAA | | |
| | | (SEQ ID NO: 5999) | | |
| KRAS | 22 | GCTGTGTCtAGAATATCCAA | RESCUE | 0.04002 |
| L56L | | GAGACAGGTT | | |
| | | (SEQ ID NO: 6000) | | |

TABLE 44
Gene-specific reverse transcription primers

| Gene | RT primer sequence |
|---|---|
| Cypridina luciferase | TTTGCATTCATCTGGTACTTCTAGGGTGTC (SEQ ID NO: 6001) |
| STAT1 | TTCATCATACTGTCGAATTCTACAGAGCCC (SEQ ID NO: 6002) |
| CTNNB1 | TTACAGGTCAGTATCAAACCAGGCCAG (SEQ ID NO: 6003) |
| STAT3 | TTTCTGCAGCTTCCGTTCTCAGCTCCTCAC (SEQ ID NO: 6004) |
| LATS1 | TACTAGATCGCGATTTTTAATCTCTGAGCC (SEQ ID NO: 6005) |
| Gaussia luciferase | TTGTCCACCTGGCCCTGGATC (SEQ ID NO: 6006) |
| KRAS | TCATCAACACCCTGTCTTGTCTTTGCT (SEQ ID NO: 6007) |

TABLE 45
Priming sequences for site-specific amplification of RNA editing target
sites

| Editing site | Forward priming sequence | Reverse priming sequence |
|---|---|---|
| Cypridina | TAAACCAGGAAAAACATGTTGCC | CGCCCTTGGTTCCTTGACCC |
| luciferase | (SEQ ID NO: 6008) | (SEQ ID NO: 6009) |
| X85W | | |
| STAT1 Y701C | AGGAAGCACCAGAGCCAATGGA | GGGGAGCAGGTTGTCTGTGGT |
| | (SEQ ID NO: 6010) | (SEQ ID NO: 6011) |
| CTNNB1 | CTGATTTGATGGAGTTGGACATOGCC | GTATCCACATCCTCTTCCTCAGGATTGC |
| T41A/T41I | (SEQ ID NO: 6012) | (SEQ ID NO: 6013) |
| STAT3 Y705C | CAGAGAGCCAGGAGCATCCTGA | TCTAAAGTGCGGGGGGACATCG |
| | (SEQ ID NO: 6014) | (SEQ ID NO: 6015) |
| LATS1 T1079A | GGAAAGCATCCTGAACATGCATTC | CGACTGCTGCTCTGAGCCTTG |
| | (SEQ ID NO: 6016) | (SEQ ID NO: 6017) |
| Gaussia | GCCAATGCCCGGAAAGCTGG | GGACTCTTTGTCGCCTTCGTAGGTG |
| luciferase C82R | (SEQ ID NO: 6018) | (SEQ ID NO: 6019) |
| KRASD30D | AGAGAGGCCTGCTGAAAATGACTG | GAGAATATCCAAGAGACAGGTTTCTCCA |
| | (SEQ ID NO: 6020) | TCA (SEQ ID NO: 6021) |
| KRAS L56L | TOGACGAATATGATCCAACAATAGAGGATTC | CTCATGTACTGGTCCCTCATTGCACTG |
| | (SEQ ID NO: 6022) | (SEQ ID NO: 6023) |
| Illumina | CTTTCCCTACACGACGCTCTTCCGATCT | GTTCAGACGTGTGCTCTTCCGATCT |
| adapters | (SEQ ID NO: 6024) | (SEQ ID NO: 6025) |

TABLE 46
Plasmids used in this example

| | | Expression | |
|---|---|---|---|
| Name | Description | system | Link to mop |
| pAB1865 | pACYC184 pJ23119-BsmbI-B-t1 DR | Bacterial | benchling.com/soumvakannan/f/lib_ |
| | | | DpBKgseF-case13b-t- |
| | | | paper/seq_62xVVPFv-pab1865- |
| | | | pacyc184-bsmbi-sites-eliminated- |
| | | | pab1497-pj23119-bsmbi-bt1dr/edit |
| pAB1866 | pACYC184 pJ23119-BsmbI-B-t2 DR | Bacterial | benchling.com/soumvakannan/f/lib_ |
| | | | DpBKgseF-case13b-t- |
| | | | paper/seq_bnxEAxUE-pab1866- |
| | | | pacyc184-bsmbi-sites-eliminated- |
| | | | pab1497-pj23119-bsmbi-bt1dr/edit |
| pAB1867 | pACYC184 pJ23119-BsmbI-B-t3 DR | Bacterial | benchling.com/soumvakannan/f/lib_ |
| | | | DpBKgseF-case13b-t- |
| | | | paper/seq_Ax8ivqt7-pab1867- |
| | | | pacyc184-bsmbi-sites-eliminated- |
| | | | pab1497-pj23119-bsmbi-bt1dr/edit |
| pAB1870 | pACYC184 pJ23119-BsmbI-B-t4 DR | Bacterial | benchling.com/soumvakannan/f/lib_ |
| | | | DpBKgseF-case13b-t- |
| | | | paper/seq_Zfceg3uN-pb1870- |
| | | | pacyc184-bsmbi-sites-eliminated- |
| | | | pab1497-pj23119-bsmbi-bt1dr/edit |
| pAB1869 | pACYC184 pJ23119-BsmbI-B-t5 DR | Bacterial | benchling.com/soumvakannan/f/lib_ |
| | | | DpBKgseF-case13b-t- |
| | | | paper/seq_OOlfwpI0-pab1869- |
| | | | pacyc184-bsmbi-sites-eliminated- |
| | | | pab1497-pj23119-bsmbi-bt1dr/edit |
| pAB1898 | pBR322 pLac-Cas13b-t1 | Bacterial | benchling.com/soumvakannan/f/lib_ |
| | | | DpBKgseF-case13b-t- |
| | | | paper/seq_VfSVjMoz-pad1898- |
| | | | pbr322-plac-cas13b-t1/edit |
| pAB1899 | pBR322 pLac-Cas13b-t2 | Bacterial | benchling.com/soumvakannan/f/lib_ |
| | | | DpBKgseF-case13b-t- |
| | | | paper/seq_N24UIrcJ-pab1899- |
| | | | pbr322-plac-cas13b-t2/edit |
| pAB1900 | pBR322 pLac-Cas13b-t3 | Bacterial | benchling.com/soumvakannan/f/lib_ |
| | | | DpBKgseF-case13b-t- |
| | | | paper/seq_YIuQqfDy-pad1900- |
| | | | pbr322-plac-cas13b-t3/edit |
| pAB1903 | pBR322 pLac-Cas13b-t4 | Bacterial | benchling.com/soumvakannan/f/lib_ |
| | | | DpBKgseF-case13b-t- |
| | | | paper/seq_YOqWhMJu-pad1903- |
| | | | pbr322-plac-cas13b-t6/edit |
| pAB1902 | pBR322 pLac-Cas13b-t5 | Bacterial | benchling.com/soumvakannan/f/lib_ |
| | | | DpBKgseF-case13b-t- |
| | | | paper/seq_MSspKrlM-pad1902- |
| | | | pbr322-plac-cas13b-t5/edit |
| pAB1619 | U6-BpiI-Cas13b-t1-DR | Mammalian | benchling.com/soumvakannan/f/lib_ |
| | | | DpBKgseF-case13b-t- |
| | | | paper/seq_TGWYY9lg-pad1619- |
| | | | pab0001-bt1-crma-bpii- |
| | | | backbone/edit |
| pAB1620 | U6-BpiI-Cas13b-t3-DR | Mammalian | benchling.com/soumvakannan/f/lib_ |
| | | | DpBKgseF-case13b-t- |
| | | | paper/seq_rFtcYTlY-pad1620- |
| | | | pab0001-bt3-crma-bpii- |
| | | | backbone/edit |
| pAB1853 | U6-BpiI-Cas13b-t5-DR | Mammalian | benchling.com/soumvakannan/f/lib_ |
| | | | DpBKgseF-case13b-t- |
| | | | paper/seq_lutVJcwS-pab1853- |
| | | | pab0001-bt5-crma-bpii- |
| | | | backbone/edit |
| pAB1678 | CMV-HIVNES-GS-Cas13b-t1 | Mammalian | benchling.com/soumvakannan/f/lib_ |
| | | | DpBKgseF-case13b-t- |
| | | | paper/seq_PRzQ8zsn-pad1678-cmv- |
| | | | hivnes-gs-cas13b-t1/edit |
| pAB1679 | CMV-HIVNES-GS-Cas13b-t3 | Mammalian | benchling.com/soumvakannan/f/lib_ |
| | | | DpBKgseF-case13b-t- |
| | | | paper/seq_3eOvCOsW-pad1679-cmv- |
| | | | hivnes-gs-cas13b-t3/edit |
| pAB1891 | CMV-HIVNES-GS-Cas13b-t5 | Mammalian | benchling.com/soumvakannan/f/lib_ |
| | | | DpBKgseF-case13b-t- |
| | | | paper/seq_UxTccZab-pad1891-cmv- |
| | | | hivnes-gs-cas13b-t5/edit |
| pAB1680 | CMV-HIVNES-GS-dCas13b-t1 | Mammalian | benchling.com/soumvakannan/f/lib_ |
| | | | DpBKgseF-case13b-t- |
| | | | paper/seq_KQz5fCQV-pad1680-cmv- |
| | | | hivnes-gs-dcas13b-t1/edit |
| pAB1681 | CMV-HIVNES-GS-dCas13b-t3 | Mammalian | benchling.com/soumvakannan/f/lib_ |
| | | | DpBKgseF-case13b-t- |
| | | | paper/seq_pzAsYd2F-pad1676-cmv- |
| | | | hivnes-gs-dcas13b-t3/edit |
| pAB1676 | CMV-HIVNES-GS-dCas13bt1-(GGS)2- | Mammalian | benchling.com/soumvakannan/f/lib_ |
| | huADAR2dd(E488Q) | | DpBKgseF-case13b-t- |
| | | | paper/seq_byu4bYwb-pad1676-cmv- |
| | | | hivnes-dcas13b-t1-ggs2- |
| | | | huadar2dde488q/edit |
| pAB1677 | CMV-HIVNES-GS-dCas13bt3-(GGS)2- | Mammalian | benchling.com/soumvakannan/f/lib_ |
| | huADAR2dd(E488Q) | | DpBKgseF-case13b-t- |
| | | | paper/seq_DGoRU6JG-pad1677-cmv- |
| | | | hivnes-dcas13b-t3-ggs2- |
| | | | huadar2dde488q/edit |
| pAB1322 | CMV-HIVNES-GS-dCas13b6-(GGS)2- | Mammalian | benchling.com/soumvakannan/f/lib_ |
| | huADAR2dd(E488Q) | | DpBKgseF-case13b-t- |
| | | | paper/seq_7yvwljoN-pad1322-cmv- |
| | | | hivnes-dcas13b6-ggs2- |
| | | | huadar2dde488q/edit |
| pAB1659 | CMV-HIVNES-GS-dCas13b6-(GGS)2- | Mammalian | benchling.com/soumvakannan/f/lib_ |
| | huADAR2dd(E488Q/E620G) | | DpBKgseF-case13b-t- |
| | | | paper/seq_jA4gCw66-pad1659-cmv- |
| | | | hivnes-dcas13b6-ggs2- |
| | | | huadar2dde488q/edit |
| pAB1810 | CMV-HIVNES-GS-dCas13b6-(GGS)2- | Mammalian | benchling.com/soumvakannan/f/lib_ |
| | huADAR2dd(E488Q/E620G/Q696L) | | DpBKgseF-case13b-t- |
| | | | paper/seq_YctRWMAR-pad1810-cmv- |
| | | | hivnes-dcas13b6-ggs2- |
| | | | huadar2dde488qe620gq6961/edit |
| pAB1923 | CMV-HIVNES-GS-dCas13bt1-(GGS)2- | Mammalian | benchling.com/soumvakannan/f/lib_ |
| | huADAR2dd(E488Q/E620G/Q696L) | | DpBKgseF-case13b-t- |
| | | | paper/seq_CUCH3Mno-pab1923-cmv- |
| | | | hivnes-dcas13b6-ggs2- |
| | | | huadar2dde488qe620gq6961/edit |
| pAB0040 | CMV-Cluciferase(STOP85)-polyAEF1a- | Mammalian | Previously described⁹ |
| | G-luciferase-polyA | | |
| pAB1424 | CMV-Cluciferase(W113XTGA)-polyA | Mammalian | benchling.com/soumvakannan/f/lib_ |
| | EFla-G-luciferase-polyA | | DpBKgseF-case13b-t- |
| | | | paper/seq_cbalO5Gr-pab1424-cmv- |
| | | | c-luciferasew113x-tga-polya-ef1a-g- |
| | | | luciferase-polya/edit |
| pAB1230 | pYES3/CTpADHl-HH-BsmBI-B6-DR- | Yeast | benchling.com/soumvakannan/f/lib_ |
| | HDV-ADH1-termTGA-ADE2TAG- | | DpBKgseF-case13b-t- |
| | URA3 | | paper/seq_m2cbleab-pad1230- |
| | | | pves3ct-padb1-bh-bsmbi-bsmbi-b6- |
| | | | dr-bdv-adh1-term-tga-ade2-tag-ura3- |
| | | | reporter/edit |
| pAB1417 | pGAL-dCas13b6-(GGS)2- | Yeast | benchling.com/soumvakannan/f/lib_ |
| | dADAR2(E488Q) | | DpBKgseF-case13b-t- |
| | | | paper/seq_gYojaefG-pab1417-pgal- |
| | | | dcas13b6-ggs2-dadar2e488q-leu2- |
| | | | selection/edit |
| pAB1773 | pGAL-dCas13b6-(GGS)2- | Yeast | benchling.com/soumvakannan/f/lib_ |
| | dADAR2(E488Q/E620G) | | DpBKgseF-case13b-t- |
| | | | paper/seq_5f5JRSA6-pad1773-pgal- |
| | | | dcas13b6-ggs2-dadar2e488qe620g- |
| | | | len2-selection/edit |

### Additional References

1. Abudayyeh, O. O. et al. Science 365, 382-386 (2019).
2. Chee, M. K. & Haase, S. B. G3 2, 515-526 (2012).
3. Voth, W. P., Jiang, Y. W. & Stillman, D. J. Yeast 20, 985-993 (2003).
4. Joung, J. et al. Nat. Protoc. 12, 828-863 (2017).
5. Gietz, R. D. & Schiestl, R. H. Nat. Protoc. 2, 3 1-34 (2007).
6. Gietz, R. D. & Schiestl, R. H. Nat. Protoc. 2, 38-41 (2007).
7. Matthews, M. M. et al. Nat. Struct. Mol. Biol. 23, 426-433 (2016).
8. Eggington, J. M., Greene, T. & Bass, B. L. Nat. Commun. 2, 319 (2011).
9. Cox, D. B. T. et al. Science 358, 1019-1027 (2017).

FIG. 40 shows Cas13b-t had collateral activity. Applicants evaluated fluorescence of a collateral RNAse cleavage reporter with active (WT) and catalytically inactivated (HEPN mutant) Cas13b-t3 and no protein negative control in the presence of a target or nontarget RNA species and found that, like other Cas13b, Cas13b-t cleaves RNA collaterally specifically in the presence of a target RNA species, and this collateral activity was mediated by the HEPN residues. Thus, it may be used for applications predicated upon Cas13 collateral activity, such as SHERLOCK-based diagnostics.

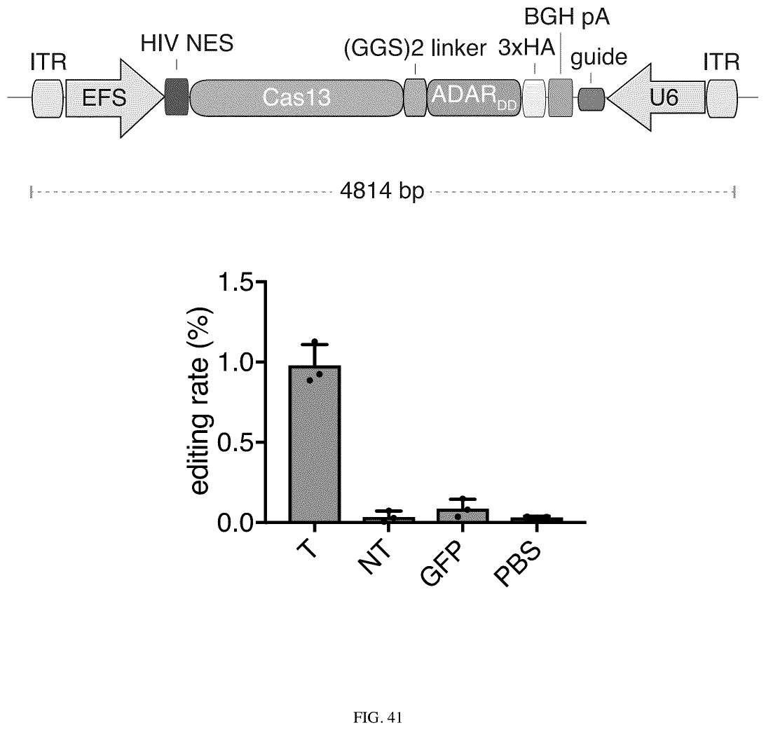

FIG. 41 shows that Cas13b-t-REPAIR mediated RNA editing via AAV delivery of a single AAV vector containing the REPAIR protein and guideRNA packaged together. Applicants packaged the construct shown in the schematic in AAV2 and delivered this to HEK293FT cells. After 2 days, Applicants evaluated RNA editing efficiency at the targeted site, the T41A codon of the CTNNB1 transcript and found that Cas13b-t-REPAIR delivered by AAV mediated RNA base editing.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A non-naturally occurring or engineered composition comprising:
(a) a Type VI Cas protein having at least 90% sequence identity to SEQ ID NO. 4628, SEQ ID NO. 4693, SEQ ID NO. 4741, SEQ ID NO. 4752, SEQ ID NO. 4753, SEQ ID NO. 5262, or SEQ ID NO. 5873; and
(b) a guide sequence capable of forming of complex with the Cas protein and directing the complex to bind to a target sequence.

2. The composition of claim 1, wherein the Cas protein is SEQ ID NO. 4628, SEQ ID NO. 4693, SEQ ID NO. 4741, SEQ ID NO. 4752, SEQ ID NO. 4753, SEQ ID NO. 5262, or SEQ ID NO. 5873

3. The composition of claim 1, wherein the Cas proteins comprises one or more mutations in a HEPN domain that reduce collateral activity.

4. The composition of claim 1, wherein the Cas protein comprises one or more mutations in a HEPN domain that renders the Cas catalytically inactive

5. The composition of claim 4, wherein the Cas protein is associated with one or more functional domains.

6. The composition of claim 5, wherein the one or more functional domains are an adenosine deaminase or a cytidine deaminase.

7. The composition of claim 6, wherein the functional domain is an adenosine deaminase.

8. A polynucleotide encoding the composition of claim 1.

9. A delivery vehicle comprising the composition of claim 1 or a polynucleotide encoding the Cas protein and/or guide molecule.

10. The delivery vehicle of claim 9, wherein the polynucleotide is a mRNA.

11. The delivery vehicle of claim 9, wherein the delivery vehicle is a lipid nanoparticle or a viral vectors, optionally, wherein the viral vector is an adeno-associated viral (AAV) vector.

12. A non-naturally occurring or engineered composition comprising:
(a) a Type VI Cas protein having at least 90% sequence identity to SEQ ID NO. 4628, SEQ ID NO. 4693, SEQ ID NO. 4741, SEQ ID NO. 4752, SEQ ID NO. 4753, SEQ ID NO. 5262, or SEQ ID NO. 5873;
(b) a nucleotide deaminase fused or linked to the Cas protein and
(b) a guide sequence capable of forming of complex with the Cas protein and directing the complex to bind to a target sequence
wherein the Cas protein is catalytically inactive and comprises an N-terminal truncation, a C-terminal truncation, or both.

13. The composition of claim 12, wherein the N-terminal or C-terminal truncation comprises at least 20 amino acids, at least 40 amino acids, at least 50 amino acids, at least 60 amino acids, at least 80 amino acids, at least 100 amino acids, at least 120 amino acids, at least 140 amino acids, at least 150 amino acids, at least 160 amino acids, at least 180 amino acids, at least 200 amino acids, at least 220 amino acids, at least 240 amino acids, at least 250 amino acids, at least 260 amino acids, or at least 300 amino acids, or at least 350 amino acids at the C-terminus and/or N-terminus of the Cas13 protein.

14. The composition of claim 13, wherein the Cas comprises a 180 amino acid N-terminal truncation and a 150 amino acid C-terminal truncation.

15. The composition of claim 12, wherein the nucleotide deaminase is an adenosine deaminase.

16. The composition of claim 12, wherein the nucleotide deaminase is a cytidine deaminase.

17. A polynucleotide encoding the composition of claim 12.

18. A delivery vehicle comprising the composition of claim 12 or a polynucleotide encoding the Cas protein and/or guide molecule.

19. The delivery vehicle of claim 18, wherein the polynucleotide is a mRNA.

20. The delivery vehicle of claim 18, wherein the delivery vehicle is a lipid nanoparticle or a viral vectors, optionally, wherein the viral vector is an adeno-associated viral (AAV) vector.

21. A method of cleaving a target RNA, comprising contacting the target RNA with the non-naturally occurring or engineered composition of claim 1; wherein the Cas protein associates with an RNA guide sequence to form a complex; wherein the complex binds to the target RNA; and wherein upon binding of the complex to the target RNA, the Cas protein cleaves the target RNA.

22. The method of claim 21, wherein the Cas protein comprises one or more mutations in a HEPN domain that eliminate or reduce a collateral activity of the Cas protein.

23. A method of editing a target RNA, comprising contacting the target RNA with the non-naturally occurring or engineered composition of claim 12, wherein the complex binds to the target RNA; and wherein upon binding of the complex to the target RNA, the Cas protein makes an A→G or C→T base edit.

24. A method of treating a disease, comprising administering the composition of claim 1, to a subject in need thereof.

25. The method of claim 24, wherein the disease is selected from cancer, haemophilia, β-thalassaemia, Marfan syndrome, Wiskott-Aldrich syndrome, Duchenne muscular dystrophy (DMD), Huntington's disease (HD), amyotrophic lateral sclerosis (ALS), macular dystrophies or degeneration (including age-related macular degeneration), retinitis pigmentosa, transthretin amyloidosis, porphyria, and primary hyperoxaluria.

26. A method of treating a disease, comprising administering the composition of claim 12.

27. The method of claim 26, wherein the Cas protein comprises a N-terminal and C-terminal truncation.

28. The method of claim 27, wherein the Cas protein comprises a 180 amino acid N-terminal truncation and a 150 amino acid C-terminal truncation.

30. The method of claim 29, wherein the disease is selected from cancer, haemophilia, Marfan syndrome, Wiskott-Aldrich syndrome, Duchenne muscular dystrophy (DMD), Huntington's disease (HD), amyotrophic lateral sclerosis (ALS), macular dystrophies or degeneration (including age-related macular degeneration), retinitis pigmentosa, sickle cell related disease (e.g., sickle cell trait, sickle cell disease such as sickle cell anemia, β-thalassaemia), alpha-1 antitrypsin deficiency, glycogen storage diseases types I-IV, Wilson's disease, cystic fibrosis, and chronic granulomatous disease.
